(19) United States
(12) Patent Application Publication
(10) Pub. No.: US 2025/0296971 A1
(43) Pub. Date: Sep. 25, 2025

(54) GASTRIC INHIBITORY PEPTIDE RECEPTOR LIGANDS

(71) Applicant: 3B PHARMACEUTICALS GMBH, Berlin (DE)

(72) Inventors: Christian HAASE, Berlin (DE); Anne SCHUMANN, Berlin (DE); Eberhard SCHNEIDER, Michendorf OT Wildenbruch (DE); Frank OSTERKAMP, Berlin (DE); Aileen HÖHNE, Berlin (DE); Ulrich REINEKE, Berlin (DE); Matthias PASCHKE, Berlin (DE); Christiane SMERLING, Berlin (DE); Jan UNGEWISS, Berlin (DE); Dirk ZBORALSKI, Berlin (DE)

(73) Assignee: 3B PHARMACEUTICALS GMBH, Berlin (DE)

(21) Appl. No.: 18/703,063

(22) PCT Filed: Oct. 25, 2022
(86) PCT No.: PCT/EP2022/079845

§ 371 Date: Apr. 19, 2024

(30) Foreign Application Priority Data
Oct. 25, 2021 (EP) 21204592.6

(51) Int. Cl. C07K 14/575; A61K 51/08; G01N 33/574; G01N 33/60

(52) U.S. Cl. CPC C07K 14/575; A61K 51/088; G01N 33/57484; G01N 33/60; A61K 2121/00; A61K 2123/00

(57) ABSTRACT

The present invention is related to compound comprising
a cyclic peptide of formula (Ia)

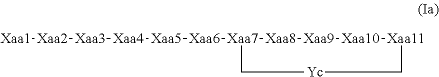

or a cyclic peptide of formula (Ib)

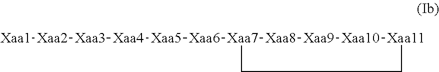

each optionally comprising an N-terminal modification group A attached to Xaa1, and each optionally comprising a C-terminal group C-term attached to Xaa11.

### CROSS REFERENCE TO RELATED APPLICATIONS

This application is a National Stage of International Application No. PCT/EP2022/079845 filed Oct. 25, 2022, claiming priority based on European Patent Application No. 21204592.6 filed Oct. 25, 2021.

### INCORPORATION BY REFERENCE OF SEQUENCE LISTING

The content of the electronically submitted sequence listing, file name: Q295539_Substitute_sequence_listing; size:1,058,000 bytes; and date of creation: Jun. 10, 2025, filed herewith, is incorporated herein by reference in its entirety.

### FIELD OF THE INVENTION

The present invention is related to a chemical compound; a peptide; a Gastric Inhibitory Peptide Receptor (GIPR) binding compound; a Gastric Inhibitory Peptide Receptor (GIPR) binding peptide; a composition comprising the compound; a composition comprising the Gastric Inhibitory Peptide Receptor (GIPR) binding compound; a composition comprising the peptide; a composition comprising the Gastric Inhibitory Peptide Receptor (GIPR) peptide; the compound, the Gastric Inhibitory Peptide Receptor (GIPR) binding compound, the peptide, the Gastric Inhibitory Peptide Receptor (GIPR) peptide and the compositions, respectively, for use in a method for the diagnosis of a disease; the compound, the Gastric Inhibitory Peptide Receptor (GIPR) binding compound and the compositions, respectively, for use in a method for the treatment of a disease; the compound, the Gastric Inhibitory Peptide Receptor (GIPR) binding compound, the peptide, Gastric Inhibitory Peptide Receptor (GIPR) peptide and the compositions, respectively, for use in a method of diagnosis and treatment of a disease which is also referred to as “thera(g)nosis” or “thera(g)nostics”; the compound, the Gastric Inhibitory Peptide Receptor (GIPR) binding compound, the peptide, the Gastric Inhibitory Peptide Receptor (GIPR) binding peptide, and the compositions, respectively, for use in a method for delivering a radionuclide to a Gastric Inhibitory Peptide Receptor (GIPR) to a cell, preferably a GIPR overexpressing tumor cell or pancreatic beta cell; a method for the diagnosis of a disease using the compound, Gastric Inhibitory Peptide Receptor (GIPR) binding compound, the peptide, the Gastric Inhibitory Peptide Receptor (GIPR) binding peptide and the compositions, respectively; a method for the treatment of a disease using the compound, the Gastric Inhibitory Peptide Receptor (GIPR) binding compound, the peptide, the Gastric Inhibitory Peptide Receptor (GIPR) binding peptide and the compositions, respectively; a method for the diagnosis and treatment of a disease which is also referred to as “thera(g)nosis” or “thera(g)nostics”, using the compound, the Gastric Inhibitory Peptide Receptor (GIPR) binding compound, the peptide, the binding peptide and the compositions, respectively; a method for the delivery of a radionuclide to a Gastric Inhibitory Peptide Receptor (GIPR) expressing tissue using the compound, the Gastric Inhibitory Peptide Receptor (GIPR) binding compound, the peptide, the Gastric Inhibitory Peptide Receptor (GIPR) binding peptide and the compositions, respectively.

### BACKGROUND

The human gastric inhibitory polypeptide (GIP) is a 42 amino acid incretin hormone (H-Tyr-Ala-Glu-Gly-Thr-Phe-Ile-Ser-Asp-Tyr-Ser-Ile-Ala-Met-Asp-Lys-Ile-His-Gln-Gln-Asn-Phe-Val-Asn-Trp-Leu-Leu-Ala-Gln-Lys-Gly-Lys-Lys- Asn-Asp-Trp-Lys-His-Asn-Ile-Thr-Gln-OH) (SEQ ID NO: 2) secreted from the enteroendocrine K-cells, which forces diet-related insulin secretion via the GIPR signaling cascade (Cho et al., Vitam Horm, 2010, 84, 111-150). It was first discovered during cholecystokinin studies by John Brown and Raymond Pederson in the 1970s (Brown et al., Scand J Gastroenterol, 1970, 5, 537-541).

The GIPR is a 7-transmembrane spanning class B1 G-protein coupled receptor (GPCR) (Fredriksson et al., Mol Pharmacol, 2003, 63, 1256-1272), which is expressed in the gastrointestinal tract and the pancreatic islet cells (Usdin et al., Endocrinology, 1993, 133, 2861-2870). The extra-cellular N-terminal domain recognizes the ligand on its middle or C-terminal part, followed by the N-terminal part docking into the transmembrane domain of the receptor (Parthier et al., Proc Natl Acad Sci USA, 2007, 104, 13942-13947). Receptor conformation induces transcription of the pro-insulin gene and insulin release by activating the adenylyl cyclase pathway that increases intracellular cyclic adenosine 3′ 5′-monophasphate (cAMP) which is associated with elevated Ca²⁺ influx (Ding et al., Diabetes, 1997, 46, 615-621). Also, proliferative and anti-apoptotic effects were triggered by activation of the mitogen-activated protein kinase (MAPK) pathway and inhibition of caspase 3 (Khan et al., Peptides, 2020, 125, 170201). Both GIP(1-42)NH₂ and GIP(1-30)NH₂ are GIPR agonists and substrates for the dipeptidyl peptidase-4 (DPP4) resulting in potent GIPR inhibitors (GIP(3-42)NH₂; GIP(3-30) NH₂). While N-terminal truncations lead to decreased receptor activation and antagonism (Hansen et al., Br J Pharmacol, 2016, 173, 826-838), C-terminal modifications have only minor impact on stability or tissue distribution.

Recently, the GIPR came into the focus for peptide receptor radionuclide therapy (PRRT). In contrast to non-cancerous tissues, the GIPR is highly expressed in several subgroups of neuroendocrine neoplasms (Waser et al., J Clin Endocrinol Metab, 2012, 97, 482-488).

In 2012, a study showing a high GIP Receptor expression in gastroenteropancreatic and bronchial neuroendocrine tumors was published (Waser et al., J Clin Endocrinol Metab, 2012, 97, 482-488). The authors claimed GIPR represents a novel molecular target for clinical applications such as in vivo scintigraphy and targeted radiotherapy. WO 2012/168464 described a method for imaging and therapy of endocrine gastroenteropancreatic tumors and bronchial and thyroid neuroendocrine tumors by targeting GIPR. The receptor's incidence and density in human neuroendocrine tumors and non-neoplastic tissues was analyzed by autoradiography. Of these tumors, functional pancreatic neuroendocrine tumors, including insulinomas, gastrinomas, glucagonomas and vipomas, as well as non-functional pancreatic NETs and ileal NETs, presented highest receptor expression. In non-neoplastic tissues the highest expression was found in islets of the human pancreas. Academic groups published studies in which radio-labelled GIP analogs were used for nuclear medicine imaging applications (Goumi et al., J Nucl Med, 2014, 55, 976-982; Willekens et al., Sci Rep, 2018, 8, 2948). As proof of concept these studies have been successful, however, tumor uptake of the labeled peptides was around 20× lower when compared to kidney uptake.

### DETAILED DESCRIPTION OF THE INVENTION

The problem underlying the present invention is the provision of a compound which is suitable as a diagnostic agent and/or a therapeutic agent, particularly if conjugated to a diagnostically and/or therapeutically active radionuclide.

A further problem underlying the present invention is the provision of a compound which is suitable as a diagnostic agent and/or a therapeutic agent, particularly if conjugated to a diagnostically and/or therapeutically active radionuclide, having a pEC₅₀ of equal to or greater than 8.0 and/or a pIC₅₀ of equal to or greater than 7.5 for Gastric Inhibitory Peptide Receptor (GIPR).

A further problem underlying the present invention is the provision of a compound which is suitable as a diagnostic agent and/or a therapeutic agent, particularly if conjugated to a diagnostically and/or therapeutically active radionuclide, in the diagnosis and/or therapy of a disease where the diseased cells and/or diseased tissues express Gastric Inhibitory Peptide Receptor (GIPR). A still further problem underlying the instant invention is the provision of a compound which is suitable for delivering a diagnostically and/or therapeutically effective radionuclide to a diseased cell and/or diseased tissue, respectively, and more particularly a GIPR-expressing diseased cell and/or diseased tissue, preferably the diseased tissue comprises cancer or tumor cells.

Also, a problem underlying the present invention is the provision of a method for the diagnosis of a disease, of a method for the treatment and/or prevention of a disease, and a method for the combined diagnosis and treatment of a disease; preferably such disease is a disease involving GIPR-expressing cells and/or tissues, more particularly a GIPR-expressing diseased cell and/or diseased tissue, preferably the diseased tissue comprises or contains cancer or tumor cells or pancreatic beta cells.

A still further problem underlying the present invention is the provision of a method for the identification of a subject, wherein the subject is likely to respond or likely not to respond to a treatment of a disease, a method for the selection of a subject from a group of subjects, wherein the subject is likely to respond or likely not to respond to a treatment of a disease; preferably, the disease is cancer, more preferably the disease is a neuroendocrine tumor.

Also, a problem underlying the present invention is the provision of a pharmaceutical composition containing a compound having the characteristics as outlined above. Furthermore, a problem underlying the present invention is the provision of a kit which is suitable for use in any of the above methods.

These and other problems are solved by the subject matter of the attached independent claims; preferred embodiments may be taken from the attached dependent claims.

These and other problems are also solved by the subject matter of the following Embodiments.

Embodiment 1. A compound comprising a cyclic peptide of formula (Ia)

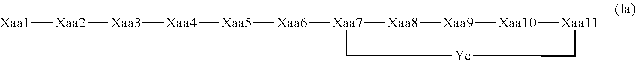

or a cyclic peptide of formula (Ib)

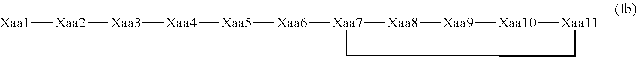

each optionally comprising an N-terminal modification group A attached to Xaa1, and each optionally comprising a C-terminal group C-term attached to Xaa11,
wherein
   the peptide sequence is drawn from left to right in N- to C-terminal direction,
   Xaa1 is a residue of an α-amino acid comprising a side chain, wherein the side chain comprises an acidic or polar group, or
   Xaa1 is a residue of an aliphatic carboxylic acid comprising an acidic or polar group at the ω-position,
   wherein the carbonyl group of Xaa1 is covalently attached to the α-nitrogen atom of residue Xaa2,
   Xaa2 is a residue of an α-amino acid comprising a side chain, wherein the side chain comprises a heteroaryl ring or an aryl ring, preferably Xaa2 is a residue of an α-amino acid with a side chain comprising a heteroaryl ring,
   wherein the carbonyl group of Xaa2 is covalently attached to the α-nitrogen atom of residue Xaa3,
   Xaa3 is a residue of an α-amino acid comprising a side chain, wherein the side chain comprises a heteroaryl ring or an aryl ring, preferably Xaa3 is a residue of an α-amino acid comprising a side chain, wherein the side chain comprises a heteroaryl ring,
   wherein the carbonyl group of Xaa3 is covalently attached to the α-nitrogen atom of residue Xaa4,
   Xaa4 is a residue of an α-amino acid optionally comprising a Z group or is a residue of an N-alkylated α-amino acid optionally comprising a Z group,
   wherein the carbonyl group of Xaa4 is covalently attached to the α-nitrogen atom of residue Xaa5,
   Xaa5 is a residue of an α-amino acid optionally comprising a Z group,
   wherein the carbonyl group of Xaa5 is covalently attached to the α-nitrogen atom of residue Xaa6,
   Xaa6 is a residue of an α-amino acid comprising a side chain, wherein the side chain comprises a branched aliphatic group,
   wherein the carbonyl group of Xaa6 is covalently attached to the α-nitrogen atom of residue Xaa7,
   Xaa7 is a residue of an α-amino acid comprising a side chain, wherein the side chain comprising a nucleophilic group, wherein the nucleophilic group comprises a heteroatom, wherein the heteroatom is selected from the group comprising a sulfur atom and a nitrogen atom,
   wherein the carbonyl group of Xaa7 is covalently attached to the α-nitrogen atom of residue Xaa8,
   Xaa8 is a residue of an α-amino acid optionally comprising a Z group,
   wherein the carbonyl group of Xaa8 is covalently attached to the α-nitrogen atom of residue Xaa9,
   Xaa9 is a residue of an α-amino acid,
   wherein the carbonyl group of Xaa9 is covalently attached to the α-nitrogen atom of residue Xaa10,
   Xaa10 is a residue of an α-amino acid comprising a side chain, wherein the side chain comprises a branched aliphatic group or an unsubstituted aromatic ring,
   wherein the carbonyl group of Xaa10 is covalently attached to the α-nitrogen atom of Xaa11, if Xaa11 is a residue of an α-amino acid comprising a side chain, wherein the side chain comprises a nucleophilic group, wherein the nucleophilic group comprises a heteroatom, wherein the heteroatom is selected from the group comprising a sulfur atom and a nitrogen atom, or, if Xaa11 is a residue of an aliphatic bis-nucleophile, wherein the first nucleophile of the bis-nucleophile is a nitrogen atom and the second nucleophile of the bis-nucleophile is a heteroatom, wherein the heteroatom is selected from the group comprising a sulfur atom and a nitrogen atom, to the first nucleophile,
   Xaa11 is a residue of an α-amino acid comprising a side chain, wherein the side chain comprises a nucleophilic group, wherein the nucleophilic group comprises a heteroatom, wherein the heteroatom is selected from the group comprising a sulfur atom and a nitrogen atom, or
   Xaa11 is a residue of an aliphatic bis-nucleophile, wherein the first nucleophile of the bis-nucleophile is a nitrogen atom and the second nucleophile of the bis-nucleophile is a heteroatom, wherein the heteroatom is selected from the group comprising a sulfur atom and a nitrogen atom,
   wherein Xaa7 and Xaa11 are indirectly linked or directly covalently linked to each other forming a macrocyclic ring,
   wherein if Xaa7 and Xaa11 are indirectly linked forming a cyclic peptide of formula (Ia), the heteroatom of Xaa7 and the heteroatom of Xaa11 are linked through a ring interspersed between the heteroatom of Xaa7 and the heteroatom of Xaa11 forming Yc, wherein the ring is a heteroaryl ring, an aryl ring optionally comprising a Z group or a heterocyclic ring, wherein the heteroatom is each and individually selected from the group consisting of a sulfur atom and a nitrogen atom, wherein if the heteroatom of Xaa7 is a sulfur atom, a thioether bond covalently links Xaa7 to the ring, if the heteroatom of Xaa7 is a nitrogen atom, an amine bond or an amide bond covalently links Xaa7 to the ring, if the heteroatom of Xaa11 is a sulfur atom, a thioether bond covalently links Xaa11 to the ring, and if the heteroatom of Xaa11 is a nitrogen atom, an amine bond or an amide bond covalently links Xaa11 to the ring, or
   wherein if Xaa7 and Xaa11 are directly covalently linked forming a cyclic peptide of formula (Ib), the heteroatom of Xaa7 is a sulfur atom and the heteroatom of Xaa11 is a sulfur atom forming a disulfide bond,
and wherein
   if present, the N-terminal modification group A is selected from the group comprising
      a) a Z group, wherein a linker is optionally interspersed between the Z group and Xaa1,
      b) a blocking group AblN,
      c) a residue of an amino acid optionally comprising a Z group and optionally comprising a blocking group AblN, and
      d) an N-terminal dipeptide group comprising a residue of a first amino acid and a residue of a second amino acid, wherein the residue of the first amino acid is the N-terminal amino acid residue and the residue of the second amino acid is the C-terminal amino acid residue,
         wherein the residue of the first amino acid optionally comprises a blocking group AblN,
         wherein the residue of the first amino acid and the residue of the second amino acid are covalently attached to each other, preferably by a peptide bond,
         wherein the residue of the second amino acid is covalently attached to the residue of Xaa1 or a linker is interspersed between the second amino acid and the residue of Xaa1,
         and wherein a Z group is optionally covalently attached to the residue of the first amino acid and/or a Z group is optionally covalently attached to the residue of the second amino acid,
and wherein
   if present, the C-terminal group C-term is selected from the group comprising
      a) a residue of an amino acid,
         wherein the residue of an amino acid is covalently attached to Xaa11 or a linker is interspersed between the residue of an amino acid and Xaa11,
         wherein a Z group is optionally covalently attached to the residue of an amino acid,
         and wherein the residue of an amino acid comprises a blocking group AblC,
      b) a C-terminal dipeptide group, wherein the C-terminal dipeptide group comprises a residue of a first amino acid and a residue of a second amino acid, wherein the residue of the first amino acid is the N-terminal amino acid residue and the residue of the second amino acid is the C-terminal amino acid residue,
         wherein the residue of the first amino acid and the residue of the second amino acid are covalently attached to each other, preferably by a peptide bond,
         wherein the residue of the first amino acid is covalently attached to the residue of Xaa11 or a linker is interspersed between the residue of the first amino acid and the residue of Xaa11,
         wherein a Z group is optionally covalently attached to the residue of the first amino acid and/or a Z group is optionally covalently attached to the residue of the second amino acid,
         and wherein the residue of the second amino acid comprises a blocking group AblC;
      c) a Z group; and
      d) a blocking group AblC.

Embodiment 2. The compound of embodiment 1,

wherein
Xaa1 is (a) a residue of an α-amino acid comprising a side chain, wherein the side chain comprises an acidic or polar group, or
(b) a residue of an aliphatic carboxylic acid comprising an acidic or polar group at the ω-position, preferably Xaa1 is a residue of an amino acid of formulae (IIa), (IIb) or (IIc), more preferably Xaa1 is a residue of an amino acid formula (IIa),

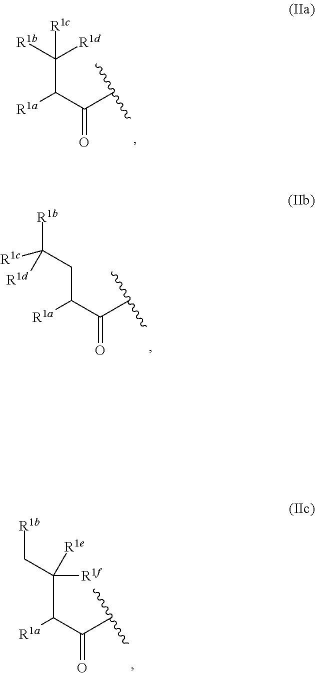

wherein
   R^{1a} is selected from the group consisting of —NH—, H, —NH((C₁-C₄)alkyl)-, OH, and (C₁-C₄)alkyl,
      wherein if R^{1a} is selected from the group consisting of —NH— and —NH((C₁-C₄)alkyl)-, the N-terminal modification group A is covalently attached to the nitrogen atom of R^{1a},
         and if R^{1a} is selected from the group consisting of H, OH or (C₁-C₄)alkyl, the N-terminal modification group A is absent,
   R^{1b} is selected from the group consisting of CO₂H, SO₃H, OPO₃H₂, CONH₂ and OH,
   R^{1c} is selected from the group consisting of H, OH and (C₁-C₂)alkyl under the proviso that R^{1b} is selected from the group consisting of CO₂H, SO₃H and CONH₂,
      or is selected from the group consisting of H and (C₁-C₂)alkyl under the proviso that R^{1b} is selected from the group consisting of OH and OPO₃H₂,
   R^{1d} is selected from the group consisting of H and (C₁-C₂)alkyl, preferably if
   R^{1e} is (C₁-C₂)alkyl, R^{1d} is the same (C₁-C₂)alkyl, R^{1c} is selected from the group consisting of H, OH and (C₁-C₂)alkyl,
   R^{1f} is selected from the group consisting of H and (C₁-C₂)alkyl, preferably if R^{1e} is (C₁-C₂)alkyl, R^{1f} is the same (C₁-C₂)alkyl,
   and the carbonyl group of Xaa1 is covalently attached to the α-nitrogen atom of residue Xaa2,
Xaa2 is (a) a residue of an α-amino acid comprising a side chain, wherein the side chain comprises a heteroaryl ring or an aryl ring, preferably Xaa2 is a residue of an amino acid of formulae (IIIa) or (IIIb), more preferably a residue of an amino acid of formula (IIIa),

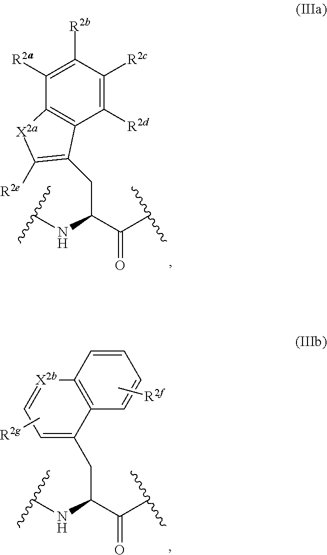

wherein
   X^{2a} is selected from the group consisting of NH, NCH₃ and S, preferably selected from the group consisting of NH and S,
   X^{2b} is selected from the group consisting of N, CH, CF and C—CH₃, preferably selected from the group consisting of N and CH,
   R^{2a} is selected from the group consisting of H, a halogen, methyl and OCH₃, preferably R^{2a} is selected from the group consisting of H and F,
   R^{2b} is selected from the group consisting of H, a halogen, methyl and OCH₃, preferably the halogen is selected from the group consisting of Cl and Br, more preferably R^{2b} is selected from the group consisting of H, Cl and Br,
   R^{2c} is selected from the group consisting of H, OH, a halogen, methyl and OCH₃, preferably the halogen is selected from the group consisting of F, Cl and Br, more preferably R², is selected from the group consisting of H, F and OH,
   R^{2d} is selected from the group consisting of H, a halogen, methyl and OCH₃, wherein the halogen is selected from the group consisting of F, Cl and Br, preferably R^{2d} is H,
   R^{2e} is selected from the group consisting of H, a halogen and methyl, preferably the halogen is Cl, more preferably R^{2e} is H,
   R^{2f} is selected from the group consisting of H and OCH₃, preferably R^{2f} is H,
   R^{2g} is selected from the group consisting of H, OH, methyl and OCH₃, preferably R^{2g} is H,
or (b), under the proviso that Xaa3 is of formulae (IVa) or (IVb), preferably under the proviso that Xaa3 is of formula (IVa), Xaa2 is a residue of an α-amino acid comprising a side chain, wherein the side chain comprises an aryl ring, preferably Xaa2 is a residue of an amino acid of formula (IIIc)

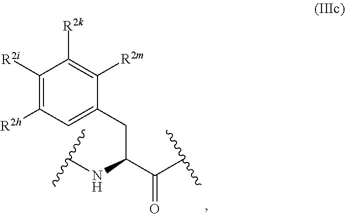

wherein
   R^{2h} is selected from the group consisting of H, methyl, CF₃, OH, OCH₃, OCF₃, CONH₂ and a halogen, preferably the halogen is selected from the group consisting of Cl and Br, more preferably R^{2h} is H, Cl or Br,
   R²ⁱ is selected from the group consisting of H, methyl, C(CH₃)₃, CF₃, OH, OCH₃, OCH₂CH₃, OCF₃, CONH₂, CO₂H and a halogen, preferably the halogen is selected from the group consisting of Cl and Br, more preferably R²ⁱ is selected from the group consisting of H, Cl and Br,
   R^{2k} is selected from the group consisting of H, methyl, CF₃, OH, OCH₃, OCF₃, CONH₂ and a halogen, preferably the halogen is selected from the group consisting of Cl and Br, more preferably R^{2k} is selected from the group consisting of H, Cl and Br,
   R^{2m} is selected from the group consisting of H, methyl, CF₃, OH, OCH₃, OCF₃, CONH₂ and a halogen, preferably the halogen is selected from the group consisting of Cl and Br, more preferably R^{2m} is selected from the group consisting of H, Cl and Br,
and the carbonyl group of Xaa2 is covalently attached to the α-nitrogen atom of residue Xaa3,
Xaa3 is (a) a residue of an α-amino acid comprising a side chain, wherein the side chain comprises a heteroaryl ring or an aryl ring, preferably Xaa3 is a residue of an amino acid of formulae (IVa) or (IVb), more preferably Xaa3 is a residue of an amino acid of formula (IVa),

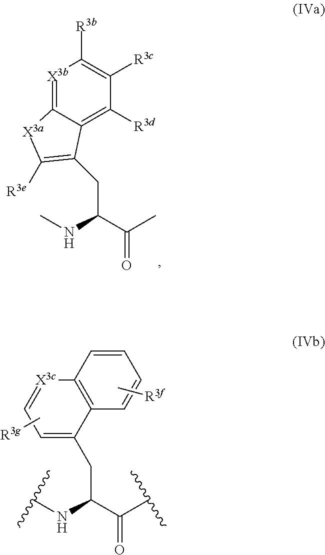

wherein
   X^{3a} is selected from the group consisting of NH, NCH₃ and S, preferably selected from the group consisting of NH and S,
   X^{3b} is selected from the group consisting of C—H, C—F, C—Cl, C—Br, C—CH₃, C—OCH₃ and N, preferably X^{3b} is C—H or C—F,
   X^{3c} is selected from the group consisting of C and N, preferably X^{3c} is C,
   R^{3b} is selected from the group consisting of H, a halogen, methyl and OCH₃, preferably the halogen is selected from the group consisting of F and Cl, more preferably R^{3b} is selected from the group consisting of H and F,
   R^{3c} is selected from the group consisting of H, a halogen, methyl and OCH₃, preferably the halogen is selected from the group consisting of Cl, Br and F, more preferably R^{3c} is selected from the group consisting of Cl and Br,
   R^{3d} is selected from the group consisting of H, a halogen, methyl and OCH₃, preferably the halogen is F,
   R^{3e} is selected from the group consisting of H, a halogen and methyl, preferably the halogen is Cl,
   R^{3f} is selected from the group consisting of H and OCH₃,
   R^{3g} is selected from the group consisting of H, F, OH, methyl and OCH₃,
or (b) under the proviso that Xaa2 is an amino acid residue of formula (IIIa), Xaa3 is a residue of an α-amino acid comprising a side chain, wherein the side chain comprises an aryl ring, preferably Xaa3 is a residue of an amino acid of formulae (IVc) or (IVd)

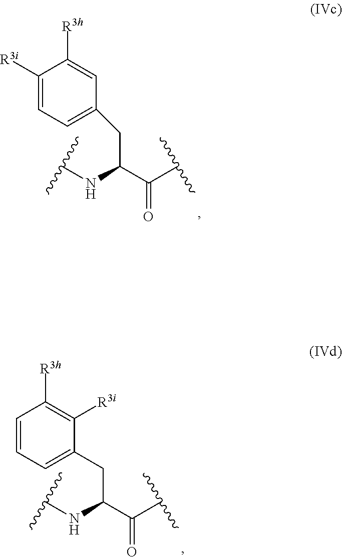

wherein
   R^{3h} is selected from the group consisting of a halogen, OH, methyl, CF₃, OCH₃, OCF₃ and CONH₂, preferably the halogen is selected from the group consisting of Cl and Br,
   R³ⁱ is selected from the group consisting of a halogen, OH, methyl, CF₃, OCH₃, OCF₃ and CONH₂, preferably the halogen is selected from the group consisting of Cl and Br,
and the carbonyl group of Xaa3 is covalently attached to the α-nitrogen atom of residue Xaa4,
Xaa4 is (a) a residue of a cyclic non-aromatic α-amino acid, preferably Xaa4 is a residue of an amino acid of formula (Va)

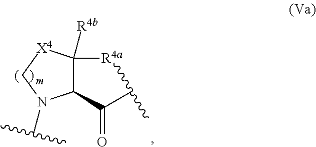

wherein
   R^{4a} is selected from the group consisting of H, OH, methyl and CF₃,
   R^{4b} is selected from the group consisting of H and methyl, preferably R^{4b} is methyl if R^{4a} is methyl,
   X⁴ is selected from the group consisting of CHR^{4c}, S and O,
      wherein R^{4c} is absent or is selected from the group consisting of NH₂, OH, H, NHR^{4d}, methyl and F, preferably R^{4c} is NH₂ or OH,
      wherein R^{4d} is selected from the group consisting of Ac and a Z group,
   m is 1 or 2, preferably m is 1,
or (b) a residue of an N-alkylated amino acid, preferably Xaa4 is a residue of an amino acid of formula (Vb)

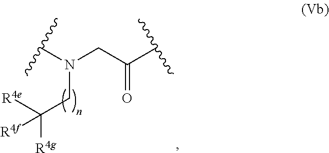

wherein
   n is 0, 1, 2, 3, 4, or 5, preferably n is 0, 1 or 3,
   R^{4e} is, if n is 0, selected from the group consisting of H, methyl, an aryl ring, COOH, CONH₂ and C(═O)R^{4h},
      or is, if n is 1, 2, 3, 4 or 5, selected from the group consisting of H, methyl, an aryl ring, OH, NH₂, COOH, CONH₂ and C(═O)R^{4h},
   R^{4f} is selected from the group consisting of H and (C₁-C₂)alkyl,
   R^{4g} is selected from the group consisting of H and methyl,
   R^{4h} is a Z group, preferably the Z group is a bio-distribution modifier, more preferably the bio-distribution modifier is AGLU,
or (c) Xaa4 is a residue of a bicyclic non-aromatic amino acid, preferably Xaa4 is a residue of an amino acid of formula (Vc)

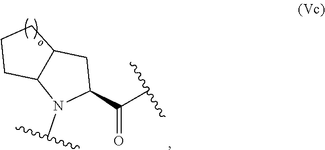

wherein
   is 1 or 2,
and the carbonyl group of Xaa4 is covalently attached to the α-nitrogen atom of residue Xaa5,
Xaa5 is (a) a residue of an α-amino acid optionally comprising a Z group, wherein the α-nitrogen atom of the α-amino acid is optionally substituted by (C₁-C₄)alkyl or (b) a cyclic α-amino acid, and the carbonyl group of Xaa5 is covalently attached to the α-nitrogen atom of Xaa6,
Xaa6 is a residue of an α-amino acid comprising a side chain, wherein the side chain comprises a branched aliphatic group, preferably Xaa6 is a residue of an amino acid of formula (VIIa)

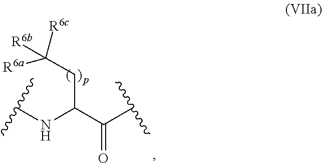

wherein
   p is 1, 0 or 2, preferably p is 1,
   R^{6a} is selected from the group consisting of (C₁-C₂)alkyl,
   R^{6b} is methyl,
   R^{6c} is H, if p is 0,
   or is selected from the group consisting of H and methyl, if p is 1 or 2,
and the carbonyl group of Xaa6 is covalently attached to the α-nitrogen atom of residue Xaa7,
Xaa7 is a residue of an α-amino acid comprising a side chain, wherein the side chain comprising a nucleophilic group, wherein the nucleophilic group comprises a heteroatom, wherein the heteroatom is selected from the group comprising a sulfur atom and a nitrogen atom, preferably Xaa7 is a residue of an α-amino acid of formula (VIIIa)

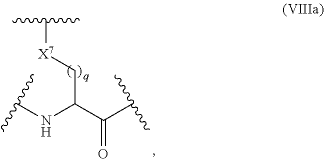

wherein
   X⁷ comprises the heteroatom and is selected from the group consisting of —S— and —NH—,
   q is 1 or 2, preferably q is 1,
and the carbonyl group of Xaa7 is covalently attached to the α-nitrogen atom of residue Xaa8,
Xaa8 is (a) a residue of an aliphatic cyclic α-amino acid, of an aliphatic heterocyclic α-amino acid or of an aliphatic cyclic α-amino acid comprising an annulated aromatic ring, preferably Xaa8 is a residue of an amino acid of formula (IXa) or (IXb), more preferably Xaa8 is a residue of an amino acid of formula (IXa),

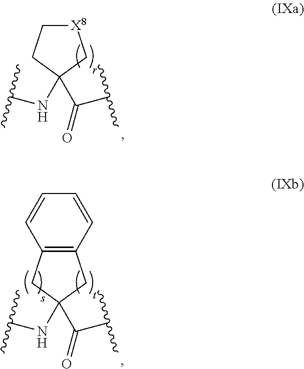

wherein
   X⁸ is selected from the group consisting of NR^{8a}, O, NH, and CH₂,
      wherein R^{8a} is a Z group or R^{8a} is acetyl,
   r is 2 or 1, preferably r is 2,
   s is 1 or 2, preferably s is 1,
   t is 1 or 2, preferably t is 1 if s is 1, and t is 2 if s is 2,
or (b) a residue of an α-amino acid or an α-alkyl-α-amino acid, wherein preferably Xaa8 is a residue of an amino acid of formula (IXc),

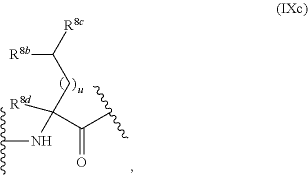

wherein
   R^{8b} is selected from the group consisting of H, OH, NH₂, NHR^{8e}, (C₁-C₂)alkyl, COOH, CONH₂, an aryl ring and a heteroaryl ring, preferably the aryl ring is phenyl and the heteroaryl ring is 3-indoyl, wherein R^{8e} is a Z group, preferably the Z group is a chelator optionally comprising a linker,
   R^{8c} is selected from the group consisting of H and methyl, if R^{8b} is selected from the group consisting of OH, NH₂ and NHR^{8e},
      or is selected from the group consisting of H, methyl and OH, if R^{8b} is selected from the group consisting of H, (C₁-C₂)alkyl, COOH, CONH₂, an aryl ring and a heteroaryl ring,
   R^{8d} is selected from the group consisting of H and (C₁-C₂)alkyl,
   u is 0, 1, 2, 3, 4, or 5, preferably u is 0, 1 or 3,
and the carbonyl group of Xaa8 is covalently attached to the α-nitrogen atom of residue Xaa9,
Xaa9 is a residue of an acyclic α-amino acid, whereby the acyclic α-amino acid is in L-configuration and the carbonyl group of Xaa9 is covalently attached to the α-nitrogen atom of Xaa10,
Xaa10 is a residue of an α-amino acid comprising a side chain, wherein the side chain comprises a branched aliphatic group or an unsubstituted aromatic ring, preferably Xaa10 is a residue of an α-amino acid of formula (XIa),

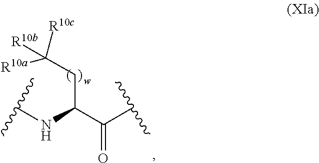

wherein
   R^{10a} is selected from the group consisting of (C₁-C₂)alkyl and phenyl,
   R^{10b} is selected from the group consisting of methyl and H,
   R^{10c} is selected from the group consisting of H and methyl,
   w is 0 or 1,
and the carbonyl group of Xaa10 is covalently attached to the α-nitrogen of Xaa11, if Xaa11 is a residue of an α-amino acid comprising a side chain, wherein the side chain comprises a nucleophilic group, wherein the nucleophilic group comprises a heteroatom, wherein the heteroatom is selected from the group comprising S and N, or, if Xaa11 is a residue of an aliphatic bis-nucleophile, wherein the first nucleophile of the bis-nucleophile is a nitrogen atom and the second nucleophile of the bis-nucleophile is a heteroatom, wherein the heteroatom is selected from the group comprising S and N, to the first nucleophile,
Xaa11 is (a) a residue of an α-amino acid comprising a side chain, wherein the side chain comprises a nucleophilic group, wherein the nucleophilic group comprises a heteroatom, wherein the heteroatom is selected form the group comprising a sulfur atom and a nitrogen atom,
or (b) Xaa11 is a residue of an aliphatic bis-nucleophile, wherein the first nucleophile of the bis-nucleophile is a nitrogen atom and the second nucleophile of the bis-nucleophile is a heteroatom, wherein the heteroatom is selected from the group comprising a sulfur atom and a nitrogen atom,
preferably Xaa11 is of formula (XIIa)

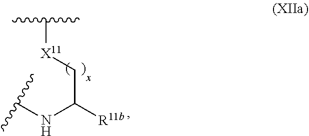

wherein
   X¹¹ comprises the heteroatom and is selected from the group consisting of —S— and —NH—,
   R^{11b} is selected from the group consisting of C(═O)R^{11c}, CONH₂, CO₂H, CH₂OH and H, wherein R^{11c} is the C-terminal group C-term,
   x is 1 or 2,
and wherein if R^{1a} in Xaa1 is selected from the group consisting of —NH— and —NH((C₁-C₄)alkyl)-, the N-terminal modification group A is covalently attached to the nitrogen atom in R^{1a}, wherein the N-terminal modification group A is selected from the group comprising
   a) a Z group, wherein a linker is optionally interspersed between the Z group and Xaa1, or
   b) a blocking group AblN, wherein the blocking group AblN is selected from the group consisting of acetyl, a structure of formula (AblN-Ia) and Nph2p,

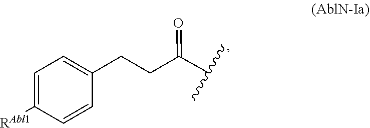

wherein
   R^{Abl1} is selected from the group consisting of H, OH and a halogen, wherein the halogen is preferably selected from the group consisting of F and Cl, preferably R^{Abl1} is OH
preferably AblN is of formula (Abl-Ia) or is acetyl, more preferably Abl is acetyl, or
c) the residue of an amino acid as defined in embodiment 1, wherein the amino acid is an α-amino acid,
   wherein the residue of an amino acid optionally comprises a Z group covalently attached to the side chain of the residue of an amino acid and optionally comprises a Z group covalently attached to the α-amino group of the residue of an amino acid, or
   wherein the residue of an amino acid optionally comprises a blocking group AblN covalently attached to the α-amino group of the residue of an amino acid, preferably the blocking group AblN is acetyl,
   and wherein the carbonyl group of the residue of an amino acid is covalently attached to the nitrogen atom in R^{1a} of residue Xaa1,
and
d) the N-terminal dipeptide group as defined in embodiment 1,
   wherein the residue of the first amino acid, the residue of the second amino acid or both the residues of the first and of the second amino acid optionally comprise a Z group,
   wherein an optional blocking group AblN is covalently attached to the α-amino group of the residue of the first amino acid,
   and wherein a linker is optionally interspersed between the residue of the second amino acid and the residue Xaa1,
   wherein if the interspersed linker is absent, the carbonyl group of the residue of the first amino acid is covalently attached to the α-nitrogen of the residue of the second amino acid and the carbonyl group of the residue of the second amino acid is covalently attached to the nitrogen atom in R^{1a},
   or wherein if the interspersed linker is present, the carbonyl group of the residue of the first amino acid is covalently attached to the α-nitrogen of the residue of the second amino acid and the carbonyl group of the residue of the second amino acid is covalently attached to a nitrogen atom of the linker and a carbonyl group of the linker is covalently attached to the nitrogen atom in R^{1a},
wherein if Xaa11 is a residue of an amino acid of formula (XIIa), wherein R^{1b} is C(═O)R^{11c}, and R^{11c} is the C-terminal group C-term, C-term is selected from the group comprising
   a) the residue of an amino acid as defined in embodiment 1, wherein the amino acid is an α-amino acid,
      optionally comprising a Z group and optionally comprising a linker interspersed between the residue of Xaa11 and the residue of an amino acid,
      wherein if the interspersed linker is absent, the carbonyl group R^{11b} of residue Xaa11 is covalently attached to the α-nitrogen of the residue of an amino acid,
      or wherein if the interspersed linker is present, the carbonyl group in R^{11b} of residue Xaa11 is covalently attached to a nitrogen atom of the linker, a carbonyl group of the linker is covalently attached to the α-nitrogen of the residue of an amino acid,
      and wherein a blocking group AblC is covalently attached to the carbonyl group of the residue of an amino acid, wherein the blocking group AblC is selected from the group consisting of NH₂ and OH, wherein the blocking group AblC preferably is NH₂, or,
   b) the C-terminal dipeptide group,
      wherein the residue of the first amino acid, the residue of the second amino acid or both the residue of the first and of the second amino acid optionally comprise a Z group,
      and wherein a linker is optionally interspersed between the residue of Xaa11 and the residue of the first amino acid,
      wherein if the interspersed linker is absent, the carbonyl group R^{11b} of residue Xaa11 is covalently attached to the α-nitrogen of the residue of the first amino acid and the carbonyl group of the residue of the first amino acid is covalently attached to the α-nitrogen of the residue of the second amino acid,
      or wherein if the interspersed linker is present, the carbonyl group R^{11b} of residue Xaa11 is covalently attached to a nitrogen atom of the linker, a carbonyl group of the linker is covalently attached to the α-nitrogen of the residue of the first amino acid and the carbonyl group of the residue of the first amino acid is covalently attached to the α-nitrogen of the residue of the second amino acid,
      and wherein a blocking group Abl is covalently attached to the carbonyl group of the residue of the second amino acid, wherein the blocking group AblC is selected from the group consisting of NH₂ and OH, wherein the blocking group AblC preferably is NH₂,
   and
   c) a Z group.

Embodiment 3. The compound of any one of embodiments 1 and 2,
wherein
   Xaa7 is a residue of formula (VIIIa)

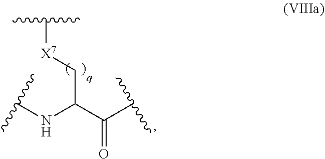

wherein
   X⁷ is —S— or —NH—,
   q is 1 or 2, preferably q is 1,
Yc is a structure of formula (XIIIa)

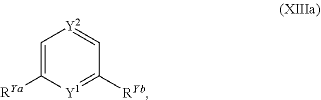

wherein
   R^{Ya} is —CH₂—,
   R^{Yb} is —CH₂—,
   Y¹ is selected from the group consisting of N and CH,
   Y² is selected from the group consisting of CH and N,
and Xaa11 is a residue of formula (XIIa)

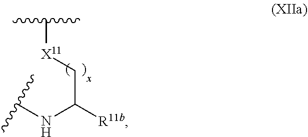

wherein
   X¹¹ is —S— or —NH—,
   R^{11b} is selected from the group consisting of C(═O)R^{11c}, CONH₂, CO₂H, CH₂OH and H, wherein R^{11c} is the C-terminal group C-term,
   x is 1 or 2, preferably x is 1,
preferably the compound comprises a cyclic peptide of formula (Ic),

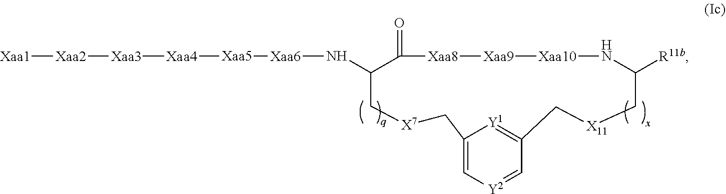

more preferably the compound comprises a cyclic peptide of formula (Ic),
wherein X⁷ is —S—, X¹¹ is —S—, Y¹ is N or CH, Y² is CH, q is 1, and x is 1,
most preferably the compound comprises a cyclic peptide of formula (Ic),
wherein X⁷ is —S—, X¹¹ is —S—, Y¹ is N, Y² is CH, q is 1, and x is 1.

Embodiment 4. The compound of embodiment 3, wherein X⁷ is —S—, X¹¹ is —S—, Y¹ is N or CH, Y² is CH, q is 1 or 2, preferably q is 1 and, x is 1 or 2, preferably x is 1.

Embodiment 5. The compound of any one of embodiments 3 and 4, wherein X⁷ is —S—, X¹¹ is —S—, Y¹ is N, Y² is CH, q is 1, and, x is 1.

Embodiment 6. The compound of embodiment 3, wherein Xaa7 is a residue of an α-amino acid selected from the group consisting of Cys, cys, Hey and Dap, wherein Xaa11 is a residue selected from the group consisting of Cys, cys, Hey, AET, Cysol, en and Dap, and wherein Yc is selected from the group consisting of 2Lut, 3MeBn and 3Lut.

Embodiment 7. The compound of any one of embodiments 3, 4 and 6, wherein Xaa7 is a Cys or Hey residue, wherein Xaa11 is a Cys or Hey residue, and wherein Yc is selected from 2Lut and 3MeBn, preferably is 2Lut.

Embodiment 8. The compound of any one of embodiments 3, 4, 5, 6 and 7, preferably the compound of embodiment 7, wherein Xaa7 is a Cys residue, wherein Xaa11 is a Cys residue, and wherein Yc is 2Lut.

Embodiment 9. The compound of any one of embodiments 3 and 6, wherein X⁷ is —NH—, X¹¹ is —S—, Y¹ is N or CH, preferably Y¹ is N, Y² is CH, q is 1 or 2, preferably q is 1 and, x is 1 or 2, preferably x is 1.

Embodiment 10. The compound of any one of embodiments 3 and 9, preferably the compound of embodiment 9, wherein Xaa7 is a Dap residue, wherein Xaa11 is a residue selected from the group consisting of Cys, cys, Hey, AET, Cysol, and wherein Yc is selected from 2Lut and 3MeBn, preferably Yc is 2Lut.

Embodiment 11. The compound of any one of embodiments 3 and 6, wherein X⁷ is —S—, X¹¹ is —NH—, Y¹ is N or CH, preferably Y¹ is N, Y² is CH, q is 1 or 2, preferably q is 1 and, x is 1

Embodiment 12. The compound of any one of embodiments 3, 6 and 11, preferably the compound of embodiment 11, wherein Xaa7 is a Cys or Hey residue, preferably Xaa7 is a Cys residue, wherein Xaa11 is an ‘en’ or Dap residue, and wherein Yc is selected from 2Lut and 3MeBn, preferably Yc is 2Lut.

Embodiment 13. The compound of any one of embodiments 3 and 6, wherein X⁷ is —NH—, X¹¹ is —NH—, Y¹ is N or CH, preferably Y¹ is N, Y² is CH, q is 1 or 2, preferably q is 1 and, x is 1 or 2, preferably x is 1.

Embodiment 14. The compound of any one of embodiments 3, 6 and 13, preferably the compound of embodiment 13, wherein Xaa7 is a Dap residue, wherein Xaa11 is an ‘en’ or a Dap residue, and wherein Yc is selected from 2Lut and 3MeBn, preferably Yc is 2Lut.

Embodiment 15. The compound of any one of embodiments 1 and 2,
wherein
   Xaa7 is a residue of formula (VIIIa)

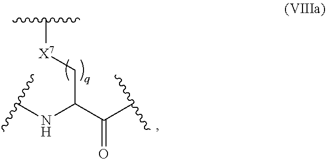

wherein
   X⁷ is —S— or —NH—,
   q is 1 or 2, preferably q is 1,
Yc is a structure of formula (XIIIh)

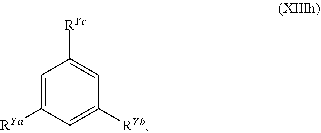

wherein
   R^{Ya} is —CH₂—,
   R^{Yb} is —CH₂—, and
   R^{Yc} is —CH₂—R^{Yd}, and
   R^{Yd} is a structure of formulae (XIIIc), (XIIId) or (XIIIe),

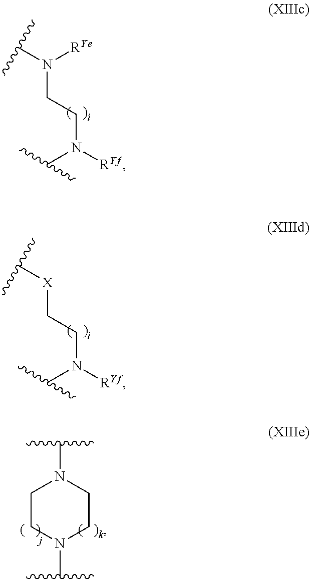

wherein
   R^{Ye} and R^{Yf} are each and independently selected from the group consisting of H and (C₁-C₄)alkyl,
   i is each and independently 1, 2, 3, 4, 5 or 6, preferably i is 1 or 2,
   j and k are each and independently 1, 2 or 3, and
X is O or S, preferably X is S,
   wherein
   in formulae (XIIIc) and (XIIIe)
   one of the two nitrogen atoms is covalently attached to —CH₂— of R^{Yc}
   and in formula (XIIId)
   —X— is covalently attached to —CH₂— of R^{Yc}
   while to the remaining nitrogen atom optionally a Z group is covalently attached, preferably the Z group comprises a chelator and optionally a linker,
and Xaa11 is a residue of formula (XIIa)

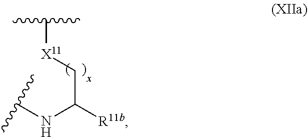

wherein
   X¹¹ is —S— or —NH—,
   R^{11b} is selected from the group consisting of C(═O)R^{11c}, CONH₂, CO₂H, CH₂OH and H, wherein R^{11c} is the C-terminal group C-term,
   x is 1 or 2, preferably x is 1,
preferably the compound comprises a cyclic peptide of formula (Ii),

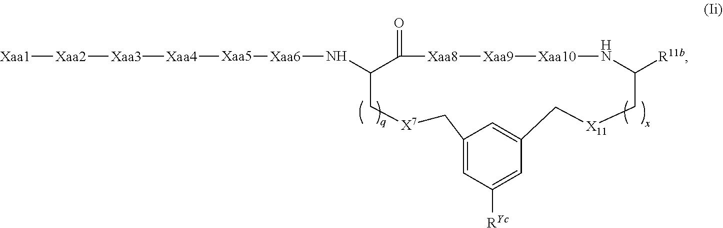

more preferably the compound comprises a cyclic peptide of formula (Ii),
   wherein
      X⁷ is —S— and X¹¹ is —S—,
      R^{Yc} is —CH₂—R^{Yd}, and
      R^{Yd} is a structure of formulae (XIIId) or (XIIIe),
      wherein R^{f} is H, i is 1, j is 1, and k is 1,
      wherein in formula (XIIId) —X— is S and is attached to —CH₂— of R^{Yc}, and in formula (XIIIe) one of the two nitrogen atoms is attached to —CH₂— of R^{Yc},
      while to the remaining nitrogen atom in either (XIIId) or (XIIIe) optionally a Z group is covalently attached, preferably the Z group comprises a chelator and optionally a linker,
      q is 1, and
      x is 1,
most preferably the compound comprises a cyclic peptide of formula (Ii), wherein R^{Yd} is a structure of formula (XIIId), wherein —X— is S and R^{f} is H, and i is 1.

Embodiment 16. The compound of embodiment 15, wherein Xaa7 is a Cys residue, wherein Xaa11 is a Cys residue, and wherein Yc is tMeBn(DOTA-AET) or tMeBn(DOTA-PP).

Embodiment 17. The compound of any one of embodiments 1 to 2,
wherein
   Xaa7 is a residue of formula (VIIIa)

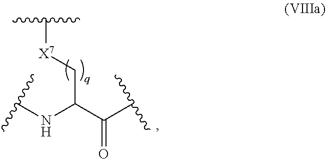

wherein
   X⁷ is —S— or —NH—,
   q is 1 or 2, preferably q is 1,
Yc is a structure of formula (XIIIb)

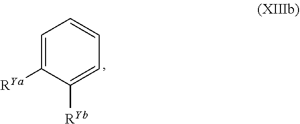

wherein
   R^{Ya} is —CH₂—,
   R^{Yb} is —CH₂—,
and Xaa11 is a residue of formula (XIIa)

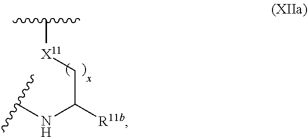

wherein
   X¹¹ is —S— or —NH—,
   R¹¹ is selected from the group consisting of C(═O)R^{11c}, CONH₂, CO₂H, CH₂OH and H, wherein R^{11c} is the C-terminal group C-term,
   x is 1 or 2, preferably x is 1,
preferably the compound comprises a cyclic peptide of formula (Id),

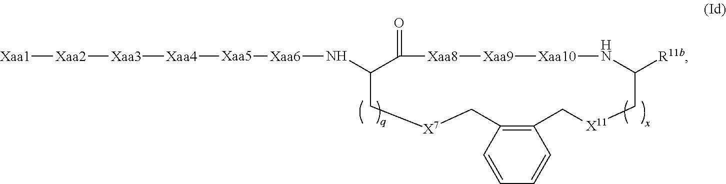

more preferably the compound comprises a cyclic peptide of formula (Id),
   wherein X⁷ is —S—, X¹¹ is —S—, q is 1, and x is 1.

Embodiment 18. The compound of embodiment 17, wherein X⁷ is —S—, X¹¹ is —S—, q is 1 or 2, preferably q is 1 and, x is 1 or 2, preferably x is 1.

Embodiment 19 The compound of any one of embodiments 17 and 18, preferably the compound of embodiment 18, wherein Xaa7 is a Cys or Hey residue, wherein Xaa11 is a Cys or Hey residue, and wherein Yc is 2MeBn.

Embodiment 20. The compound of embodiment 17, wherein X⁷ is —NH—, X¹¹ is —S—, q is 1 or 2, preferably q is 1 and, x is 1 or 2, preferably x is 1.

Embodiment 21. The compound of any one of embodiments 17 and 20, preferably the compound of embodiment 20, wherein Xaa7 is a Dap residue, wherein Xaa11 is a Cys or Hey residue, and wherein Yc is 2MeBn.

Embodiment 22. The compound of embodiment 17, wherein X⁷ is —S—, X¹¹ is —NH—, q is 1 or 2, preferably q is 1 and, x is 1 or 2, preferably x is 1.

Embodiment 23. The compound of any one of embodiments 17 and 22, preferably the compound of embodiment 22, wherein Xaa7 is a Cys or Hey residue, wherein Xaa11 is an ‘en’ or a Dap residue, and wherein Yc is 2MeBn.

Embodiment 24. The compound of embodiment 17, wherein X⁷ is —NH—, X¹¹ is —NH—, q is 1 or 2, preferably q is 1 and, x is 1 or 2, preferably x is 1.

Embodiment 25. The compound of any one of embodiments 17 and 24, preferably the compound of embodiment 24, wherein Xaa7 is a Dap residue, wherein Xaa11 is an ‘en’ or a Dap residue, and wherein Yc is 2MeBn.

Embodiment 26. The compound of any one of embodiments 1 and 2,
wherein
   Xaa7 is a residue of formula (VIIIb)

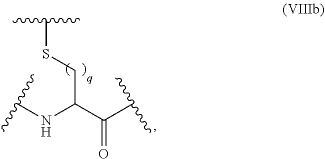

wherein
   q is 1 or 2, preferably q is 1,
Yc is a structure of formula (XIIIg)

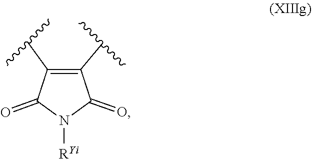

wherein
   R^{Yi} is selected from the group consisting of H and (C₁-C₆)alkyl,
and Xaa11 is a residue of formula (XIIb)

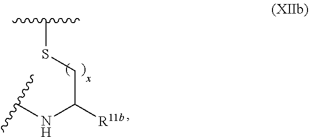

wherein
   R^{11b} is selected from the group consisting of C(═O)R^{11c}, CONH₂, CO₂H, CH₂OH and H, wherein R^{11c} is the C-terminal group C-term,
   x is 1 or 2, preferably x is 1,
preferably the compound comprises a cyclic peptide of formula (Ig),

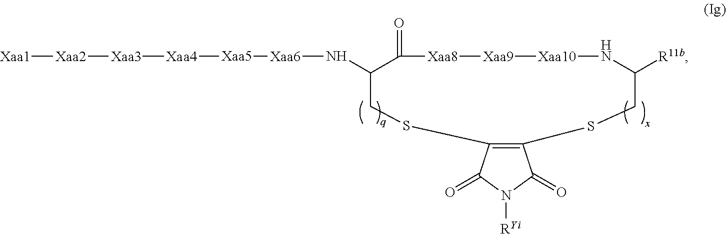

more preferably the compound comprises a cyclic peptide of formula (Ig),
   wherein q is 1, and x is 1 or 2.

Embodiment 27. The compound of embodiment 26, wherein Xaa7 is a Cys residue, wherein Xaa7 is selected from the group comprising Cys, Hey, Cysol and AET, and wherein Yc is mli.

Embodiment 28. The compound of any one of embodiments 1 and 2,
wherein
   Yc is a structure of formula (XIIIf)

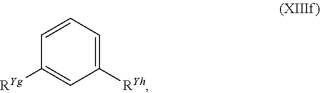

wherein
   R^{Yg} is —C(═O)—,
   R^{Yh} is —CH₂—,
Xaa7 is a residue of formula (VIIIa)

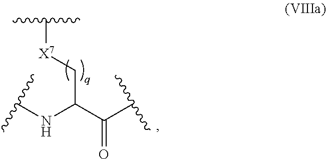

wherein
   X⁷ is —S— or —NH— and is covalently attached to R^{Yh},
   q is 1 or 2, preferably q is 1,
and Xaa11 is a residue of formula (XIIa)

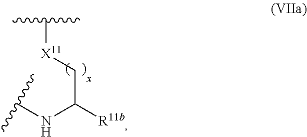

wherein
   X¹¹ is —NH— and is covalently attached to R^{Y9},
   R^{11b} is selected from the group consisting of C(═O)R^{11c}, CONH₂, CO₂H, CH₂OH and H, wherein R^{11c} is the C-terminal group C-term,
   x is 1 or 2, preferably x is 1,
preferably the compound comprises a cyclic peptide of formula (If),

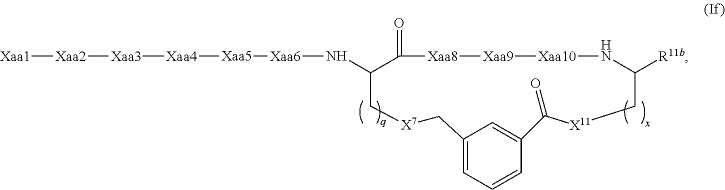

more preferably the compound comprises a cyclic peptide of formula (If),
   wherein X⁷ is —S—, q is 1, and x is 1.

Embodiment 29. The compound of embodiment 28, wherein X⁷ is —S—, q is 1 or 2, preferably q is 1 and, x is 1 or 2, preferably x is 1.

Embodiment 30. The compound of any one of embodiments 28 and 29, preferably the compound of embodiment 29, wherein Xaa7 is a Cys or Hey residue, wherein Xaa11 is an ‘en’ or a Dap residue, and wherein Yc is 3CbBn.

Embodiment 31. The compound of embodiment 28, wherein X⁷ is —NH—, q is 1 or 2, preferably q is 1 and, x is 1 or 2, preferably x is 1.

Embodiment 32. The compound of any one of embodiments 28 and 30, preferably the compound of embodiment 30, wherein Xaa7 is a Dap residue, wherein Xaa11 is an ‘en’ or a Dap residue, and wherein Yc is 3CbBn.

Embodiment 33. The compound of any one of embodiments 1 and 2,
wherein
   Yc is a structure of formula (XIIIf)

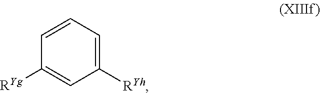

wherein
   R^{Yg} is —C(═O)—,
   R^{Yh} is —CH₂—,
Xaa7 is a residue of formula (VIIIa)

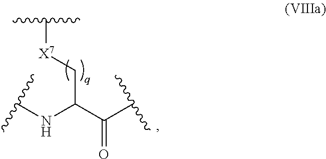

wherein
   X⁷ is —NH— and is covalently attached to R^{Yg},
   q is 1 or 2, preferably q is 1,
and Xaa11 is a residue of formula (XIIa)

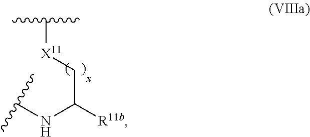

wherein
   X¹¹ is —S— or —NH— and is covalently attached to R^{Yh},
   R^{11b} is selected from the group consisting of C(═O)R^{11c}, CONH₂, CO₂H, CH₂OH and H, wherein R^{11c} is the C-terminal group C-term,
   x is 1 or 2, preferably x is 1,
preferably the compound comprises a cyclic peptide of formula (Ie),

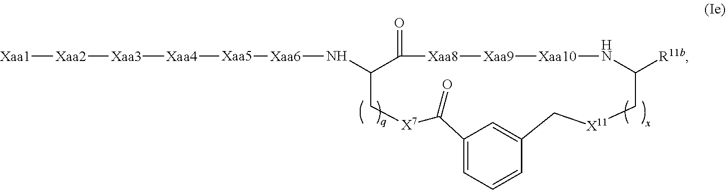

more preferably the compound comprises a cyclic peptide of formula (Ie),
   wherein X¹¹ is —S—, q is 1, and x is 1.

Embodiment 34. The compound of embodiment 33, wherein X¹¹ is —S—, q is 1 or 2, preferably q is 1 and, x is 1 or 2, preferably x is 1.

Embodiment 35. The compound of any one of embodiments 33 and 34, preferably the compound of embodiment 34, wherein Xaa7 is a Dap residue, wherein Xaa11 is Cys or Hey residue, and wherein Yc is 3MeBz.

Embodiment 36. The compound of embodiment 33, wherein X¹¹ is —NH—, q is 1 or 2, preferably q is 1 and, x is 1 or 2, preferably x is 1.

Embodiment 37. The compound of any one of embodiments 33 and 36, preferably the compound of embodiment 36, wherein Xaa7 is a Dap residue, wherein Xaa11 is an ‘en’ or a Dap residue, and wherein Yc is 3MeBz.

Embodiment 38. The compound of any one of embodiments 1 and 2,
wherein
   the compound comprises a cyclic peptide of formula (Ib),
   Xaa7 is a residue of formula (VIIIc)

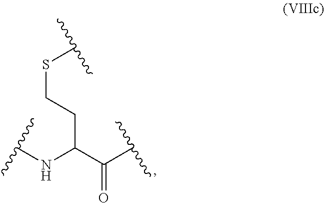 [SMILES: *NC(CCS*)C(*)=O]

and Xaa11 is a residue of formula (XIIc)

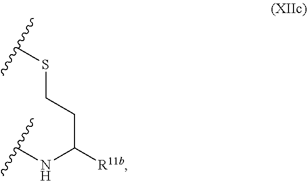

wherein
   R^{11b} is selected from the group consisting of C(═O)R^{11c}, CONH₂, CO₂H, CH₂OH and H, wherein R^{11c} is the C-terminal group C-term,
preferably the compound comprises a cyclic peptide of formula (Ih),

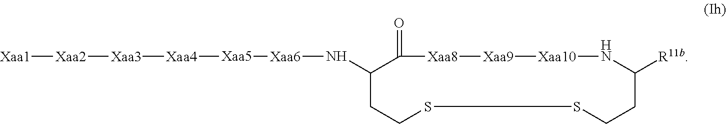

Embodiment 39. The compound of embodiment 38, wherein Xaa7 is a Hey residue and Xaa11 is a Hey residue.

Embodiment 40. The compound of any one of embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38 and 39, wherein Xaa1 is a residue of an α-amino acid comprising a side chain, wherein the side chain comprises an acidic or polar group within the side chain or a residue of an aliphatic carboxylic acid comprising an additional acidic or polar group at the ω-position, of formula (IId) or (IIe), preferably Xaa1 is a residue of an amino acid formula (IId),

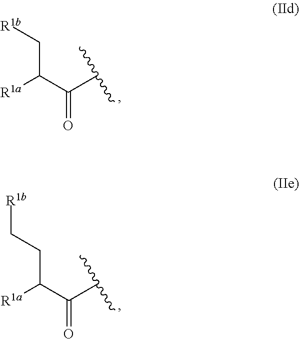

wherein
   R^{1a} is selected from the group consisting of —NH—, H or —N(CH₃)—,
   R^{1b} is selected from the group consisting of CO₂H, CONH₂, OH, SO₃H and OPO₃H₂,
   under the proviso that R^{1a} is —NH— or —N(CH₃)—, the N-terminal modification group A is covalently attached to the nitrogen atom of R^{1a}.

Embodiment 41. The compound of any one of embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 and 40, wherein Xaa1 is a residue of an α-L-amino acid comprising a side chain, wherein the side chain comprises an acidic or polar group within the side chain or a residue of an aliphatic carboxylic acid comprising an additional acidic or polar group at the ω-position, of formula (IIf) or (IIg), more preferably Xaa1 is a residue of an amino acid formula (IIf),

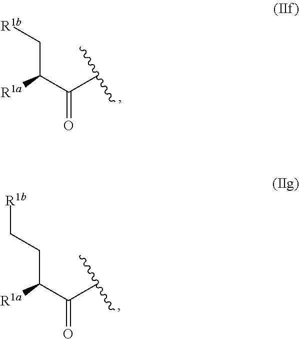

wherein
   R^{1a} is selected from the group consisting of —NH—, H or —N(CH₃)—,
   R^{1b} is selected from the group consisting of CO₂H, CONH₂, OH, SO₃H and OPO₃H₂,
under the proviso that R^{1a} is —NH— or —N(CH₃)—, the N-terminal modification group A is covalently attached to the nitrogen atom of R^{1a}.

Embodiment 42. The compound of any one of embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 and 41, preferably the compound of embodiment 41, wherein Xaa1 is a residue of an α-L-amino acid comprising a side chain, wherein the side chain comprises an acidic or polar group within the side chain or a residue of an aliphatic carboxylic acid comprising an additional acidic or polar group at the ω-position, of formula (IIf) or (IIg), more preferably Xaa1 is a residue of an amino acid formula (IIf),
wherein
   R^{1a} is —NH— or H,
   R^{1b} is selected from the group consisting of CO₂H, CONH₂, OH and SO₃H,
   under the proviso that R^{1a} is —NH—, the N-terminal modification group A is covalently attached to the nitrogen atom of R^{1a}.

Embodiment 43. The compound of any one of embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41 and 42 preferably the compound of embodiment 42, wherein Xaa1 is a residue of an α-L-amino acid comprising a side chain, wherein the side chain comprises an acidic or polar within the side chain or a residue of an aliphatic carboxylic acid comprising an additional acidic or polar group at the ω-position, of formula (IIf) or (IIg), more preferably Xaa1 is a residue of an amino acid of formula (IIf),
wherein
   R^{1a} is —NH—,
   R^{1b} is CO₂H or CONH₂,
   the N-terminal modification group A is covalently attached to the nitrogen atom of R^{1a}.

Embodiment 44. The compound of any one of embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 and 41, wherein Xaa1 is a residue of an α-L-amino acid selected from the group consisting of Asp, Asn, Glu, Gln, Hse, Cya, Pse, Nmd and Ser, or a residue of a dicarboxylic acid selected from the group consisting of Succinyl and Glutar.

Embodiment 45. The compound of any one of embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42 and 44, preferably the compound of embodiment 44, wherein Xaa1 is a residue of an α-L-amino acid selected from the group consisting of Asp, Asn, Glu, Gln, Hse, Cya, and Ser, or a residue of a dicarboxylic acid residue selected from the group consisting of Succinyl and Glutar.

Embodiment 46. The compound of any one of embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44 and 45, preferably the compound of any one of embodiments 44 and 45, wherein Xaa1 is a residue of an α-L-amino acid selected from the group consisting of Asp, Asn, Glu, and Gln.

Embodiment 47. The compound of any one of embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45 and 46, preferably the compound of any one of embodiments 44, 45 and 46, wherein Xaa1 is an Asp residue.

Embodiment 48. The compound of any one of embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46 and 47, wherein Xaa2 is a residue of an α-L-amino acid of formula (IIId),

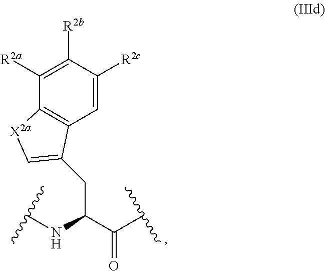

wherein
   X^{2a} is NH or S, preferably is NH,
   R^{2a} is H or F,
   R^{2b} is selected from the group consisting of H, F and Cl,
   R^{2c} is selected from the group consisting of H, F, Cl, Br and OH.

Embodiment 49. The compound of any one of embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47 and 48, preferably the compound of embodiment 47, wherein Xaa2 is a residue of an α-L-amino acid of formula (IIId),
wherein
   X^{2a} is NH,
   R^{2a} is H or F,
   R^{2b} is H,
   R^{2c} is H, F or OH.

Embodiment 50. The compound of any one of embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47 and 48, wherein Xaa2 is a residue of an α-L-amino acid selected from the group consisting of Trp, Hyw, 5Fw, 6Clw, 7Fw, Bta, 5Clw, 5Brw and 6Fw.

Embodiment 51. The compound of any one of embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48 and 50, preferably the compound of embodiment 50, wherein Xaa2 is a residue of an α-L-amino acid selected from the group consisting of Trp, Hyw, 5Fw, 6Clw, 7Fw, 5Clw, 5Brw and 6Fw.

Embodiment 52. The compound of any one of embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 and 51, preferably the compound of any one of embodiments 50 and 51, wherein Xaa2 is a residue of an α-L-amino acid selected from the group consisting of Trp, Hyw, 5Fw and 7Fw.

Embodiment 53. The compound of any one of embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51 and 52, preferably the compound of any one of embodiments 50, 51 and 52, wherein Xaa2 is a Trp residue.

Embodiment 54. The compound of any one of embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46 and 47 wherein Xaa2 is 1Ni.

Embodiment 55. The compound of any one of embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46 and 47, wherein under the proviso that Xaa3 is of formulae (IVa) or (IVb), preferably under the proviso that Xaa3 is of formula (IVa), Xaa2 is a residue of an amino acid of formula (IIIc)

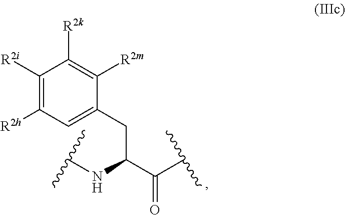

wherein
   R^{2h} is H or Cl,
   R²ⁱ is H or Cl,
   R^{2k} is H or Cl,
   R^{2m} is H or Cl.

Embodiment 56. The compound of any one of embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47 and 55, preferably the compound of embodiment 55, wherein under the proviso that Xaa3 is of formula (IVa) or (IVb), preferably under the proviso that Xaa3 is of formula (IVa), Xaa2 is a residue of an α-L-amino acid selected from the group consisting of Phe, Ocf, Mcf, Pcf, Eaa, Egm, Egn and Egp.

Embodiment 57. The compound of any one of embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 55 and 56, preferably the compound of any one of embodiments 55 and 56, wherein under the proviso that Xaa3 is of formulae (IVa) or (IVb), preferably under the proviso that Xaa3 is of formula (IVa), Xaa2 is a residue of an α-L-amino acid selected from the group consisting of Mcf and Egp.

Embodiment 58. The compound of any one of embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56 and 57, wherein Xaa3 is a residue of an amino acid of formula (IVe) or (IVf),

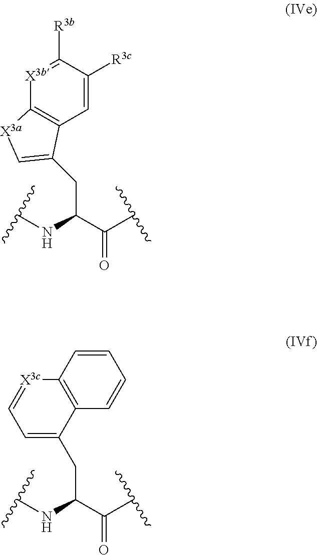

wherein
   R^{3b} is H or F,
   R^{3c} is selected from the group consisting of Cl, Br, H and F,
   X^{3a} is NH or S,
   X^{3b} is selected from the group consisting of CH, CF and N, and
   X^{3c} is CH or N, preferably X^{3c} is CH.

Embodiment 59. The compound of any one of embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57 and 58, preferably the compound of embodiment 58, wherein Xaa3 is a residue of an amino acid of formula (IVg)

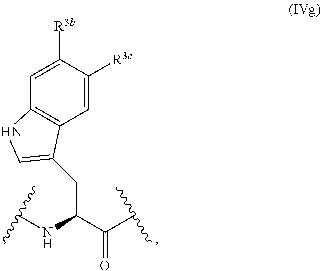

wherein
   R^{3b} is H or F, and
   R^{3c} is selected from the group consisting of Cl, Br, and H.

Embodiment 60. The compound of any one of embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56 and 57, wherein Xaa3 is a residue of an α-L-amino acid selected from the group consisting of 5Cw, 5Brw, Trp, 1Ni, Bta, 5Fw, 6Fw, 7Fw, 6Clw and 7Nw.

Embodiment 61. The compound of any one of embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58 and 60, preferably the compound of any one of embodiments 58 and 60, wherein Xaa3 is a residue of an α-L-amino acid selected from the group consisting of 5Clw, 5Brw, Trp, 1Ni, Bta, 5Fw, 6Fw and 7Fw.

Embodiment 62. The compound of any one of embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 and 61, preferably the compound of any one of embodiments 58, 59, 60 and 61, wherein Xaa3 is a residue of an α-L-amino acid selected from the group consisting of 5Clw and 5Brw.

Embodiment 63. The compound of any one of embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61 and 62, preferably the compound of any one of embodiments 58, 59, 60, 61 and 62, wherein Xaa3 is a 5Clw residue.

Embodiment 64. The compound of any one of embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53 and 54, wherein under the proviso that Xaa2 is of formulae (IIIa) or (IIIb), preferably under the proviso that Xaa2 is of formula (IIIa), Xaa3 is a residue of an amino acid of formulae (IVc) or (IVd)

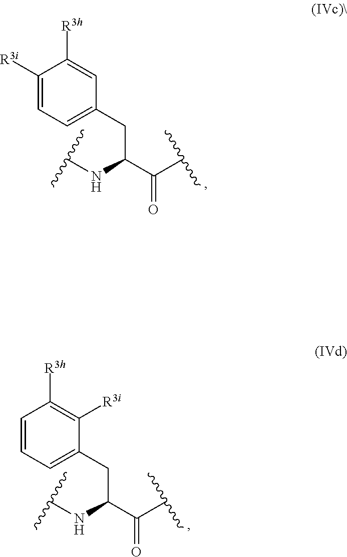

wherein R³ⁱ and R^{3h} each and independently is Cl.

Embodiment 65. The compound of any one of embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54 and 64, preferably the compound of embodiment 64, wherein under the proviso that Xaa2 is of formulae (IIIa) or (IIIb), preferably under the proviso that Xaa2 is of formula (IIIa), Xaa3 is Eaa or Egp.

Embodiment 66. The compound of any one of embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64 and 65, wherein Xaa4 is a residue of an amino acid of formula (Va)

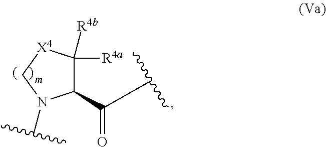

wherein
   m is 1 or 2, preferably m is 1,
   R^{4a} is selected from the group consisting of H, OH and methyl,
   R^{4b} is either H or methyl, preferably R^{4b} is methyl if R^{4a} is methyl,
   X⁴ is selected from the group consisting of CHR^{4c}, CH₂, S and O,
      wherein R^{4c} is selected from the group consisting of NHR^{4d}, NH₂, H, OH, methyl and F,
      wherein R^{4d} is Ac or is a Z group, preferably if the Z group is a chelator optionally comprising a linker, more preferably the chelator is DOTA, and if the Z group is a bio-distribution modifier optionally comprising a linker, more preferably the bio-distribution modifier is AGLU and the optional linker preferably is Glutar.

Embodiment 67. The compound of any one of embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65 and 66, preferably the compound of embodiment 66, wherein Xaa4 is a residue of an amino acid of formula (Vd)

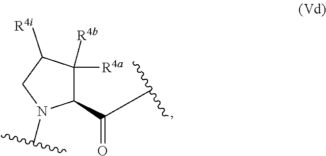

wherein
   R^{4a} is selected from the group consisting of H, OH and methyl,
   R^{4b} is H or methyl, preferably R^{4b} is methyl if R^{4a} is methyl,
   R⁴ⁱ is selected from the group consisting of NH₂, OH, NHR^{4d}, H and F,
      wherein R^{4d} is H, Ac, or is a Z group, if the Z group is a chelator comprising an optional linker, preferably the chelator is DOTA and if the Z group is a bio-distribution modifier comprising an optional linker, preferably the bio-distribution modifier is AGLU and the optional linker preferably is Glutar.

Embodiment 68. The compound of any one of embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66 and 67, preferably the compound of any one of embodiments 66 and 67, wherein Xaa4 is a residue of an amino acid of formula (Ve)

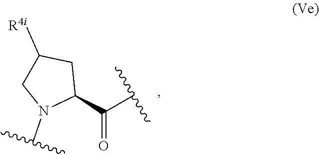

wherein
   R⁴ⁱ is selected from the group consisting of NH₂, OH and H.

Embodiment 69. The compound of any one of embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65 and 66, preferably the compound of embodiment 66, wherein Xaa4 is a residue of an α-L-amino acid selected from the group consisting of Tap, Hyp, Pro, 4Ap, 4Ap(Ac), Tap(DOTA), Tap(AGLU-Gutar), 4Tfp, H3p, Eaz, Oxa, Dtc and Pip.

Embodiment 70. The compound of any one of embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66 and 69, preferably the compound of any one of embodiments 66 and 69, wherein Xaa4 is a residue of an α-L-amino acid selected from the group consisting of Tap, Hyp, Pro, 4Ap, 4Ap(Ac), Tap(DOTA), Tap(AGLU-Gutar), 4Tfp, H3p, Eaz, Oxa and Dtc.

Embodiment 71. The compound of any one of embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 69 and 70, preferably the compound of any one of embodiments 66, 67, 69 and 70, wherein Xaa4 is a residue of an α-L-amino acid selected from the group consisting of Tap, Hyp, Pro, 4Ap, 4Ap(Ac), Tap(DOTA), Tap(AGLU-Gutar), 4Tfp and H3p.

Embodiment 72. The compound of any one of embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 69, 70 and 71, preferably the compound of any one of embodiments 66, 67, 68, 69, 70 and 71, wherein Xaa4 is a residue of an α-L-amino acid selected from the group consisting of Tap, Hyp, Pro and 4Ap.

Embodiment 73. The compound of any one of embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 69, 70, 71 and 72, preferably the compound of any one of embodiments 66, 67, 68, 69, 70, 71 and 72, wherein Xaa4 is a Tap residue.

Embodiment 74. The compound of any one of embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 69, 70, 71 and 72, preferably the compound of any one of embodiments 66, 67, 68, 69, 70, 71 and 72, wherein Xaa4 is a Hyp residue.

Embodiment 75. The compound of any one of embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 69, 70, 71 and 72, preferably the compound of any one of embodiments 66, 67, 68, 69, 70, 71 and 72, wherein Xaa4 is a Pro residue.

Embodiment 76. The compound of any one of embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64 and 65, wherein Xaa4 is a residue of an N-alkylated amino acid of formula (Vf)

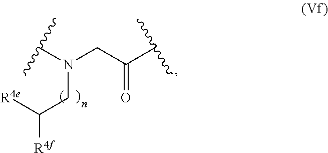

wherein
   n is 0, 1 or 3,
   R^{4e} is selected from the group consisting of H and phenyl, if n=0,
      or is NH₂, if n=3,
      or is selected from the group consisting of methyl, CO₂H, and C(═O)R^{4h}, if n=1, wherein R^{4h} is a Z group, preferably the Z group is a bio-distribution modifier optionally comprising a linker, wherein the bio-distribution modifier more preferably is AGLU, and
   R^{4f} is selected from the group consisting of H and methyl.

Embodiment 77. The compound of any one of embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65 and 76, preferably the compound of embodiment 76, wherein Xaa4 is a residue of an N-alkylated amino acid selected from the group consisting of Nmg, Nlys, Nleu, Nphe, Nglu and Nglu(AGLU).

Embodiment 78. The compound of any one of embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64 and 65, wherein Xaa4 is an Oic residue.

Embodiment 79. The compound of any one of embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77 and 78, wherein Xaa5 is a residue of an α-amino acid optionally comprising a Z group, wherein the α-nitrogen atom of the α-amino acid is optionally substituted by a methyl group.

Embodiment 80. The compound of any one of embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78 and 79, preferably the compound of embodiment 79, wherein Xaa5 is of formulae (VIa) or (IVb), preferably Xaa5 is a residue of formula (VIa),

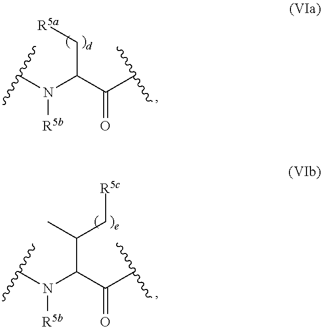

wherein
   R^{5a} is selected from the group comprising H, COOH, SO₃H, OPO₃H, CONH₂, OH, NH₂, NHR^{5d}, NHC(═NH)NH₂, an aryl ring and a heteroaryl ring, preferably the aryl ring is phenyl and the heteroaryl ring is 3-indoyl or 4-imidazoyl, and (C₁-C₄)alkyl, preferably R^{5a} is COOH,
      wherein R^{5d} is a Z group,
   R^{5b} is selected from the group of H and methyl, preferably R^{5b} is H,
   d is 1, 2, 3, 4, or 5, preferably d is 2,
   R^{5c} is selected from the group comprising H and OH,
   e is 1 or 2.

Embodiment 81. The compound of any one of embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79 and 80, preferably the compound of any one of embodiments 79 and 80, wherein Xaa5 of formula (VIa),
wherein
   R^{5a} is selected from the group comprising COOH, CONH₂, OH, NH₂, NHC(═NH)NH₂ and a heteroaryl ring, preferably the heteroaryl ring is 3-indoyl or 4-imidazoyl, preferably R^{5a} is COOH,
   d is 1, 2, 3, 4, or 5, preferably d is 2,
   R^{5b} is H.

Embodiment 82. The compound of any one of embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80 and 81, preferably the compound of any one of embodiments 79, 80 and 81, wherein Xaa5 is of formula (VIa),
wherein
   R^{5a} is selected from the group comprising COOH, CONH₂, OH, preferably R^{5a} is COOH,
   d is 1, or 2, preferably d is 2.

Embodiment 83. The compound of any one of embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81 and 82, preferably the compound of any one of embodiments 80, 81 and 82, more preferably the compound of embodiment 82, wherein the stereo configuration of Xaa5 is the L-configuration or is the D-configuration, preferably the stereo configuration of Xaa5 is the L-configuration.

Embodiment 84. The compound of any one of embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79 and 80, preferably the compound of any one of embodiments 79 and 80, wherein Xaa5 is a residue of an α-amino acid selected from the group consisting of Glu, Gln, Asp, Asn, Ser, Hse, Pse, Nme, glu, Ala, Trp, Lys, Arg, His, Hly, Har, Val and Phe.

Embodiment 85. The compound of any one of embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 83 and 84, preferably the compound of any one of embodiments 79, 80, 81, 83 and 84, wherein Xaa5 is a residue of an α-L-amino acid selected from the group consisting of Glu, Gln, Asp, Asn, Ser, Hse, Trp, Lys, Arg, His, Hly, and Har.

Embodiment 86. The compound of any one of embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 83, 84 and 85, preferably the compound of any one of embodiments 79, 80, 81, 83, 84 and 85, wherein Xaa5 is a residue of an α-L-amino acid selected from the group consisting of Glu, Gln, Asp, Asn, Ser, and Hse.

Embodiment 87. The compound of any one of embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 83, 84, 85 and 86, preferably the compound of any one of embodiments 76, 77, 78, 79, 80, 81, 83, 84, 85 and 86, wherein Xaa5 is a Glu residue.

Embodiment 88. The compound of any one of embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77 and 78, wherein, Xaa5 is a residue of a cyclic α-L-amino acid, preferably Xaa5 is a Pro residue.

Embodiment 89. The compound of any one of embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 83, 84, 85, 86, 87 and 88, wherein Xaa6 is a residue of an amino acid of formula (VIIa)

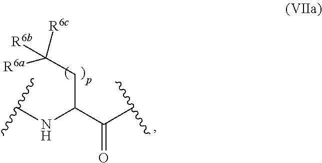

wherein
   p is 1 or 0, preferably p is 1,
   R^{6a} is (C₁-C₂)alkyl,
   R^{6b} is methyl,
   R^{6c} is H if p is 0, and is H or methyl, if p is 1.

Embodiment 90. The compound of any one of embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 83, 84, 85, 86, 87, 88 and 89, preferably the compound of embodiment 89, wherein the stereo configuration of Xaa6 is the L-configuration or is the D-configuration, preferably the stereo configuration of Xaa6 is the D-configuration.

Embodiment 91. The compound of any one of embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 83, 84, 85, 86, 87, 88, 89 and 90, preferably the compound of any one of embodiments 89 and 90, wherein Xaa6 is a residue of an α-amino acid selected from the group consisting of leu, Leu, Npg, Val, Ile and Hle.

Embodiment 92. The compound of any one of embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 83, 84, 85, 86, 87, 88, 89, 90 and 91, preferably the compound of any one of embodiments 89, 90 and 91, wherein Xaa6 is a residue of an α-amino acid selected from the group consisting of leu, Leu, Npg, Val and Ile.

Embodiment 93. The compound of any one of embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 83, 84, 85, 86, 87, 88, 89, 90, 91 and 92, preferably the compound of any one of embodiments 89, 90, 91 and 92, wherein Xaa6 is residue of a leu residue.

Embodiment 94. The compound of any one of embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 83, 84, 85, 86, 87, 88, 89, 90, 91, 92 and 93, preferably the compound of any one of embodiments 83 and 90, wherein the stereo configuration of Xaa5 is the L-configuration and the stereo configuration of Xaa6 is the D-configuration, or the stereo configuration of Xaa5 is the D-configuration and the stereo configuration of Xaa6 is the L-configuration, preferably the stereo configuration of Xaa5 is the L-configuration and the stereo configuration of Xaa6 is the D-configuration.

Embodiment 95. The compound of any one of embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 83, 84, 85, 86, 87, 88, 89, 90, 91, 92, 93 and 94, wherein Xaa8 is a residue of an amino acid of formula (IXa),

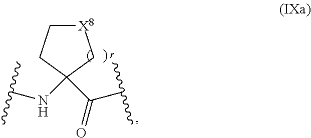

wherein
   r is 2 or 1, preferably r is 2,
   X⁸ is selected from the group consisting of NR^{8a}, O, NH, and CH₂, wherein R^{8a} is a Z group or R^{8a} is acetyl, wherein if the Z group is a chelator optionally comprising a linker linker, preferably the chelator is DOTA, or if the Z-group is a bio-distribution modifier optionally comprising a linker linker, preferably the bio-distribution modifier is AGLU and the linker preferably is Glutar, or if the Z group is a combination of a chelator optionally comprising a linker, a bio-distribution modifier optionally comprising a linker, and an amino acid residue linking the chelator optionally comprising a linker and the bio-distribution modifier optionally comprising a linker, preferably the chelator is DOTA and the bio-distribution modifier preferably is AGLU and the amino acid residue linking the chelator and the bio-distribution modifier preferably is glu residue.

Embodiment 96. The compound of any one of embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 83, 84, 85, 86, 87, 88, 89, 90, 91, 92, 93, 94 and 95, preferably the compound of embodiment 95, wherein Xaa8 is a residue of an amino acid of formula (Ixd)

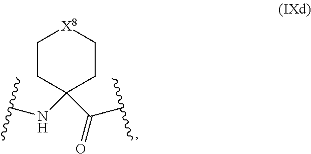

wherein
   X⁸ is selected from the group consisting of NR^{8a}, O, and NH,
      wherein R^{8a} is a Z group or R^{8a} is acetyl, wherein if the Z group is a chelator optionally comprising a linker, preferably the chelator is DOTA, or if the Z-group is a bio-distribution modifier optionally comprising a linker, preferably the bio-distribution modifier is AGLU and the linker preferably is Glutar.

Embodiment 97. The compound of any one of embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 83, 84, 85, 86, 87, 88, 89, 90, 91, 92, 93, 94, 95 and 96, preferably the compound of any one of embodiments 95 and 96, wherein Xaa8 is a residue of an amino acid of formula (IXd), wherein
X⁸ is NR^{8a} or O,
   wherein R^{8a} is a Z group or R^{8a} is acetyl, wherein if the Z group is a chelator optionally comprising a linker, preferably the chelator is DOTA.

Embodiment 98. The compound of any one of embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 83, 84, 85, 86, 87, 88, 89, 90, 91, 92, 93, 94 and 95, preferably the compound of embodiment 95, wherein Xaa8 is a residue an α-amino acid selected from the group consisting of Apc(R^{8a}), Ape, Thp, Egz, and Eca, wherein R^{8a} is a Z group, wherein if the Z group is a chelator optionally comprising a linker, preferably the chelator is DOTA, or if the Z-group is a bio-distribution modifier optionally comprising a linker, preferably the bio-distribution modifier is AGLU and the linker preferably is Glutar, or if the Z group is a combination of a chelator optionally comprising a linker, a bio-distribution modifier optionally comprising a linker, and an amino acid residue linking the chelator optionally comprising a linker and the bio-distribution modifier optionally comprising a linker, preferably the chelator is DOTA and the bio-distribution modifier preferably is AGLU and the amino acid residue linking the chelator and the bio-distribution modifier preferably is a glu residue, or R^{8a} is acetyl.

Embodiment 99. The compound of any one of embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 83, 84, 85, 86, 87, 88, 89, 90, 91, 92, 93, 94, 95, 96 and 98, preferably the compound of any one of embodiments 95, 96 and 98, wherein Xaa8 is a residue of an α-amino acid residue selected from the group consisting of Apc(R^{8a}), Apc, and Thp, wherein R^{8a} is a Z group, wherein if the Z group is a chelator optionally comprising a linker, preferably the chelator is DOTA, or if the Z-group is a bio-distribution modifier optionally comprising a linker, preferably the bio-distribution modifier is AGLU and the linker preferably is Glutar, or R^{8a} is acetyl.

Embodiment 100. The compound of any one of embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 83, 84, 85, 86, 87, 88, 89, 90, 91, 92, 93, 94, 95, 96, 97, 98 and 99, preferably the compound of any one of embodiments 95, 96, 97, 98 and 99, wherein Xaa8 is Apc(R^{8a}), wherein R^{8a} is a Z group, wherein the Z group is a chelator optionally comprising a linker, wherein the chelator
a) preferably is selected from the group consisting of DOTA, DOTAGA, DOTAM, DOTP, NOTA, NODAGA, PSC, NODA-MPAA, HBED, TETA, CB-TE2A, DTPA, CHX-A″-DTPA, DFO, Macropa, HOPO, TRAP, THP, DATA, NOPO, NOTP, PCTA, sarcophagine, FSC, NETA, NE3TA, H4octapa, pycup, HYNIC, NxS4-x (N4, N2S2, N3S), ^{99m}Tc(CO)3-chelators and their analogs,
b) more preferably is selected from the group consisting of DOTA, DOTAGA, DOTAM, NOTA, NODAGA, PSC, NODA-MPAA, NOPO, HBED, DTPA, CHX-A″-DTPA, CB-TE2A, Macropa, PCTA, N4, and analogs thereof,
and
c) most preferably is selected from the group consisting of DOTA, DOTAGA, DOTAM, NOTA, NODAGA, PSC, NOPO, Macropa, PCTA and analogs thereof.

Embodiment 101. The compound of any one of embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 83, 84, 85, 86, 87, 88, 89, 90, 91, 92, 93, 94, 95, 96, 97, 98, 99 and 100, preferably the compound of any one of embodiments 95, 96, 97, 98, 99 and 100, most preferably the compound of embodiment 100, wherein the chelator is DOTA.

Embodiment 102. The compound of any one of embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 83, 84, 85, 86, 87, 88, 89, 90, 91, 92, 93, 94, 95, 96, 98 and 99, preferably the compound of any one of embodiments 95, 96, 98 and 99, wherein Xaa8 is Apc(R^{8a}), wherein R^{8a} is a Z group, wherein the Z group is a bio-distribution modifier optionally comprising a linker, wherein the Z group
a) preferably is selected from the group consisting of AGLU with Glutar as linker, NTASEROL, GLUSEROL with Glutar as linker, AHOL with Glutar as linker, APOL with Glutar as linker, ABOL with Glutar as linker, APrOL with Glutar as linker, MeO2kDaPEGAc, MeO10kDaPEGAc and MeO36PEG,
b) more preferably is AGLU with Glutar as linker or NTASEROL,
and
c) most preferably is AGLU with Glutar as linker.

Embodiment 103. The compound of any one of embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 83, 84, 85, 86, 87, 88, 89, 90, 91, 92, 93, 94 and 95, preferably the compound of embodiment 95, wherein Xaa8 is Apc(R^{8a}) wherein R^{8a} is a Z group, wherein the Z group is a combination of a chelator optionally comprising a linker, a bio-distribution modifier optionally comprising a linker, and an amino acid residue linking the chelator optionally comprising a linker and the bio-distribution modifier optionally comprising a linker, preferably the chelator is DOTA and the bio-distribution modifier is AGLU and the amino acid linking the chelator and the bio-distribution modifier is glu, wherein the combination is of formula (IXe),

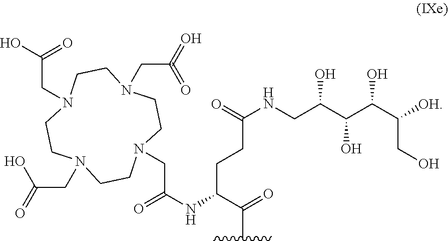

Embodiment 104. The compound of any one of embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 83, 84, 85, 86, 87, 88, 89, 90, 91, 92, 93 and 94, wherein Xaa8 is an Aic residue.

Embodiment 105. The compound of any one of embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 83, 84, 85, 86, 87, 88, 89, 90, 91, 92, 93 and 94, wherein Xaa8 is a residue of an α-amino acid selected from the group consisting of Lys, Lys(R^{8e}), lys(R^{8e}), Amk(R^{8e}), Dab(R^{8e}) and dab(R^{8e}), wherein R^{8e} is a Z group, preferably the Z group is a chelator optionally comprising a linker.

Embodiment 106. The compound of any one of embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 83, 84, 85, 86, 87, 88, 89, 90, 91, 92, 93, 94 and 105, preferably the compound of embodiment 105, wherein Xaa8 is a residue of an α-amino acid selected from the group consisting of Lys(R^{8e}), lys(R^{8e}), Amk(R^{8e}), Dab(R^{8e}) and dab(R^{8e}), wherein R^{8e} is a Z group, wherein the Z group is a chelator optionally comprising a linker, wherein the chelator
a) preferably is selected from the group consisting of DOTA, DOTAGA, DOTAM, DOTP, NOTA, NODAGA, PSC, NODA-MPAA, HBED, TETA, CB-TE2A, DTPA, CHX-A″-DTPA, DFO, Macropa, HOPO, TRAP, THP, DATA, NOPO, NOTP, PCTA, sarcophagine, FSC, NETA, NE3TA, H4octapa, pycup, HYNIC, NxS4-x (N4, N2S2, N3S), ^{99m}Tc(CO)3-chelators and their analogs,
b) more preferably is selected from the group consisting of DOTA, DOTAGA, DOTAM, NOTA, NODAGA, PSC, NODA-MPAA, NOPO, HBED, DTPA, CHX-A″-DTPA, CB-TE2A, Macropa, PCTA, N4, and analogs thereof,
and
c) most preferably is selected from the group consisting of DOTA, DOTAGA, DOTAM, NOTA, NODAGA, PSC, NOPO, Macropa, PCTA and analogs thereof.

Embodiment 107. The compound of any one of embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 83, 84, 85, 86, 87, 88, 89, 90, 91, 92, 93 and 94, wherein Xaa8 is a residue of an α-amino acid residue selected from the group consisting of Aib, Deg, Ams, ala, Ala, Gln, Thr, Trp, Phe and Glu.

Embodiment 108. The compound of any one of embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 83, 84, 85, 86, 87, 88, 89, 90, 91, 92, 93, 94, 95, 96, 97, 98, 99, 100, 101, 102, 103, 104, 105, 106 and 107, wherein Xaa9 is a residue of an amino acid of formula (Xa)

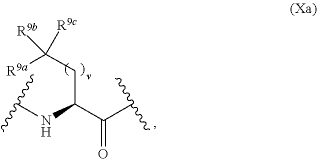

wherein
   R^{9a} is selected from the group consisting of (C₁-C₄)alkyl, COOH, CONH₂, OH, NH₂, NHC(═NH)NH₂, an aryl ring and a heteroaryl ring, preferably the aryl ring is phenyl and the heteroaryl ring is 3-indoyl, and cyclo-hexyl,
   R^{9b} is methyl or H,
   R^{9c} is H or methyl, and
   v is 0, 1, 2, preferably v is 1.

Embodiment 109. The compound of any one of embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 83, 84, 85, 86, 87, 88, 89, 90, 91, 92, 93, 94, 95, 96, 97, 98, 99, 100, 101, 102, 103, 104, 105, 106, 107 and 108, preferably the compound of embodiment 108, wherein Xaa9 is a residue of an amino acid of formula (Xa), wherein
R^{9a} is selected from the group consisting of (C₁-C₂)alkyl, COOH, OH, NHC(═NH)NH₂, an aryl ring and a heteroaryl ring, preferably the aryl ring is phenyl and the heteroaryl ring is 3-indoyl, and cyclo-hexyl,
R^{9b} is methyl or H,
R^{9c} is H, and
v is 0, 1, 2, preferably v is 1.

Embodiment 110. The compound of any one of embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 83, 84, 85, 86, 87, 88, 89, 90, 91, 92, 93, 94, 95, 96, 97, 98, 99, 100, 101, 102, 103, 104, 105, 106, 107 and 108, preferably the compound of embodiment 108, wherein Xaa9 is a residue of an α-L-amino acid selected from the group consisting of Leu, Hle, Ile, Arg, Trp, Glu, Phe, Ser, Cha, Npg and Tle.

Embodiment 111. The compound of any one of embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 83, 84, 85, 86, 87, 88, 89, 90, 91, 92, 93, 94, 95, 96, 97, 98, 99, 100, 101, 102, 103, 104, 105, 106, 107, 108, 109 and 110, preferably the compound of any one of embodiments 108, 109 and 110, wherein Xaa9 is a residue of an α-L-amino acid selected from the group consisting of Leu, Hle, Ile, Arg, Trp, Glu, Phe and Ser.

Embodiment 112. The compound of any one of embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 83, 84, 85, 86, 87, 88, 89, 90, 91, 92, 93, 94, 95, 96, 97, 98, 99, 100, 101, 102, 103, 104, 105, 106, 107, 108, 109, 110 and 111, preferably the compound of any one of embodiments 108, 109, 110 and 111, wherein Xaa9 is a Leu residue.

Embodiment 113. The compound of any one of embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 83, 84, 85, 86, 87, 88, 89, 90, 91, 92, 93, 94, 95, 96, 97, 98, 99, 100, 101, 102, 103, 104, 105, 106, 107, 108, 109, 110, 111 and 112, wherein Xaa10 is a residue of an amino acid of formula (XIa)

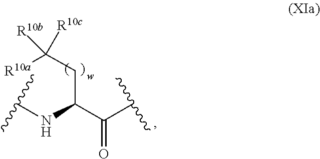

wherein
   R^{10a} is selected from the group consisting of (C₁-C₂)alkyl and phenyl, preferably R^{10a} is (C₁-C₂)alkyl, more preferably (C₁-C₂)alkyl is ethyl,
   R^{10b} is selected from the group consisting of methyl and H, preferably R^{10b} is methyl,
   R^{10c} is selected from the group consisting of H and methyl, R^{10C} is H, if w is 1, preferably R^{10c} is H, and
   w is 0 or 1, preferably w is 0.

Embodiment 114. The compound of any one of embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112 and 113, preferably the compound of embodiment 113, wherein Xaa10 is a residue of an amino acid of formula (XIa), wherein
R^{10a} is selected from (C₁-C₂)alkyl, preferably is ethyl,
R^{10b} is selected from the group consisting of methyl and H, preferably R^{10b} is methyl,
R^{10c} is selected from the group consisting of H and methyl, R^{10C} is H, if w is 1, preferably R^{10c} is H, and
w is 0 or 1, preferably w is 0.

Embodiment 115. The compound of any one of embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112 and 113, preferably the compound of embodiment 113, wherein Xaa10 is a residue of an α-L-amino acid selected from the group consisting of Ile, Leu, Tle, Val, and Phe.

Embodiment 116. The compound of any one of embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114 and 115, preferably the compound of any one of embodiments 113, 114 and 115, wherein Xaa10 is a residue of an α-L-amino acid selected from the group consisting of Ile, Leu and Tle.

Embodiment 117. The compound of any one of embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115 and 116, preferably the compound of any one of embodiments 113, 114, 115 and 116, wherein Xaa10 is an Ile residue.

Embodiment 118. The compound of any one of embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116 and 117, preferably the compound of any one of embodiments 40, 41, 42 and 43, wherein, if R^{1a} in Xaa1 is —NH— or —N(CH₃)—, or the compound of any one of embodiments 44, 45, 46 and 47, wherein, if Xaa1 is a residue of an amino acid selected from the group consisting of Asp, Asn, Glu, Gln, Hse, Cya, Pse, Nmd, and Ser, the N-terminal modification group A is covalently attached to R^{1a} in Xaa1.

Embodiment 119. The compound of any one of embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117 and 118, preferably the compound of embodiment 118, wherein the N-terminal modification group A is selected from the group comprising
a) a Z group, wherein a linker is optionally interspersed between the Z group and Xaa1, wherein
   the Z group is a chelator optionally comprising a linker,
   or the Z group is a bio-distribution modifier optionally comprising a linker,
   or the Z group is a combination of
      a chelator optionally comprising a linker,
      a bio-distribution modifier optionally comprising a linker a linker,
   and an amino acid residue linking the chelator optionally comprising a linker and the bio-distribution modifier optionally comprising a linker,
b) a blocking group AblN, wherein the blocking group AblN is selected from the group consisting of acetyl and a structure of formula (AblN-Ia),

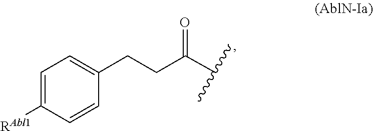

wherein
   R^{Abl1} is selected from the group consisting of H, OH and a halogen, preferably the halogen is selected from the group consisting of F and Cl, preferably R^{Abl1} is OH
preferably AblN is selected from the group consisting of Ac, 4OHPhp, 4FPhp, 4ClPhp and Php,
c) the residue of an amino acid, wherein the amino acid is an α-amino acid, wherein the residue of an amino acid
   optionally comprises a Z group covalently attached to the side chain of the residue of an amino acid and optionally comprises a Z group covalently attached to the α-amino group of the residue of an amino acid,
   or wherein the residue of an amino acid optionally comprises a blocking group AblN covalently attached to the α-amino group of the residue of an amino acid, preferably the blocking group AblN is acetyl,
   wherein the residue of an amino acid preferably is a Tyr residue, with an optional Z group covalently attached to the α-amino group of the Tyr residue, wherein the Z group is a chelator comprising an optional linker, wherein the chelator preferably is DOTA and the linker preferably is Ttds,
d) the N-terminal dipeptide group,
   wherein the residue of the first amino acid, the residue of the second amino acid or both the residue of the first and of the second amino acid optionally comprise a Z group,
   wherein a blocking group AblN is optionally covalently attached to the α-amino group of the residue of the first amino acid,
   and wherein a linker is optionally interspersed between the residue of the second amino acid and the residue of Xaa1,
   wherein if the interspersed linker is absent, the carbonyl group of the residue of the first amino acid is covalently attached to the α-nitrogen of the residue of the second amino acid and the carbonyl group of the residue of the second amino acid is covalently attached to the nitrogen atom in R^{1a} or the α-nitrogen atom of residue Xaa1,
   or wherein if the interspersed linker is present, the carbonyl group of the residue of the first amino acid is covalently attached to the α-nitrogen of the residue of the second amino acid and the carbonyl group of the residue of the second amino acid is covalently attached to a nitrogen atom of the linker and a carbonyl group of the linker is covalently attached to the nitrogen atom in R^{1a} or the α-nitrogen atom of residue Xaa1,
   wherein the first amino acid residue is a Met residue and the residue of the second amino acid is selected from the group comprising Ala, Tyr and tyr.

Embodiment 120. The compound of any one of embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118 and 119, preferably the compound of any one of embodiments 118 and 119, wherein the N-terminal modification group A is a Z group, wherein
a linker is optionally interspersed between the Z group and the residue of Xaa1, and wherein the Z group is a chelator optionally comprising a linker,
or the Z group is a bio-distribution optionally comprising a linker,
or the Z group is a combination of
   a chelator optionally comprising a linker, a bio-distribution modifier optionally comprising a linker, and an amino acid residue linking the chelator optionally comprising a linker and the bio-distribution modifier optionally comprising a linker and a linker is optionally interspersed between the amino acid residue linking the chelator optionally comprising a linker and the bio-distribution modifier optionally comprising a linker, and the residue of Xaa1, preferably the optional linker is APAc, O2Oc or PEG6.

Embodiment 121. The compound of any one of embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119 and 120, preferably the compound of any one of embodiments 118, 119 and 120, more preferably the compound of embodiment 120, wherein the Z group is a bio-distribution modifier optionally comprising a linker, wherein the Z group is
a) preferably is selected from the group consisting of AGLU with Glutar as linker, NTASEROL, GLUSEROL with Glutar as linker, AHOL with Glutar as linker, APOL with Glutar as linker, ABOL with Glutar as linker, APrOL with Glutar as linker, MeO2kDaPEGAc, MeO10kDaPEGAc and MeO36PEG,
b) more preferably is AGLU with Glutar as linker or NTASEROL, and
c) most preferably is AGLU with Glutar as linker.

Embodiment 122. The compound of any one of embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120 and 121, preferably the compound of any one of embodiments 118, 119, 120 and 121, more preferably the compound of embodiment 121, wherein the Z group is a bio-distribution modifier optionally comprising a linker, wherein the bio-distribution modifier is AGLU and the linker is Glutar.

Embodiment 123. The compound of any one of embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119 and 120, preferably the compound of any one of embodiments 118, 119 and 120, more preferably the compound of embodiment 120, wherein the Z group is a chelator optionally comprising a linker, wherein the chelator
a) preferably is selected from the group consisting of DOTA, DOTAGA, DOTAM, DOTP, NOTA, NODAGA, PSC, NODA-MPAA, HBED, TETA, CB-TE2A, DTPA, CHX-A″-DTPA, DFO, Macropa, HOPO, TRAP, THP, DATA, NOPO, NOTP, PCTA, sarcophagine, FSC, NETA, NE3TA, H4octapa, pycup, HYNIC, NxS4-x (N4, N2S2, N3S), ^{99m}Tc(CO)3-chelators and their analogs,
b) more preferably is selected from the group consisting of DOTA, DOTAGA, DOTAM, NOTA, NODAGA, PSC, NODA-MPAA, NOPO, HBED, DTPA, CHX-A″-DTPA, CB-TE2A, Macropa, PCTA, N4, and analogs thereof,
and
c) most preferably is selected from the group consisting of DOTA, DOTAGA, DOTAM, NOTA, NODAGA, PSC, NOPO, Macropa, PCTA and analogs thereof.

Embodiment 124. The compound of any one of embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120 and 123, preferably the compound of any one of embodiments 118, 119, 120 and 123, more preferably the compound of embodiment 123, wherein the Z group is a chelator optionally comprising a linker, wherein the chelator is DOTA with an optional linker, preferably the linker is APAc or Ttds.

Embodiment 125. The compound of any one of embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119 and 120, preferably the compound of embodiment 120, wherein the Z group is a combination of a chelator optionally comprising a linker, a bio-distribution modifier optionally comprising a linker, and an amino acid residue linking the chelator optionally comprising a linker and the bio-distribution modifier optionally comprising a linker, wherein the chelator
a) preferably is selected from the group consisting of DOTA, DOTAGA, DOTAM, DOTP, NOTA, NODAGA, PSC, NODA-MPAA, HBED, TETA, CB-TE2A, DTPA, CHX-A″-DTPA, DFO, Macropa, HOPO, TRAP, THP, DATA, NOPO, NOTP, PCTA, sarcophagine, FSC, NETA, NE3TA, H4octapa, pycup, HYNIC, NxS4-x (N4, N2S2, N3S), ^{99m}Tc(CO)3-chelators and their analogs,
b) more preferably is selected from the group consisting of DOTA, DOTAGA, DOTAM, NOTA, NODAGA, PSC, NODA-MPAA, NOPO, HBED, DTPA, CHX-A″-DTPA, CB-TE2A, Macropa, PCTA, N4, and analogs thereof,
and
c) most preferably is selected from the group consisting of DOTA, DOTAGA, DOTAM, NOTA, NODAGA, PSC, NOPO, Macropa, PCTA and analogs thereof and,
the bio-distribution modifier
   a) preferably is selected from the group consisting of AGLU, GLUSEROL, AHOL, APOL, ABOL, APrOL,
   and
   b) more preferably is AGLU,
and wherein the amino acid residue linking the chelator optionally comprising a linker and the bio-distribution modifier optionally comprising a linker preferably is a glu residue,
and wherein an optional linker is interspersed between the amino acid residue linking the chelator optionally comprising a linker and the bio-distribution modifier optionally comprising a linker, and the residue of Xaa1, preferably the linker is APAc, O2Oc or PEG6.

Embodiment 126. The compound of any one of embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120 and 125, preferably the compound of embodiment 125, wherein the Z group is a combination of a chelator optionally comprising a linker, a bio-distribution modifier optionally comprising a linker, and an amino acid residue linking the chelator optionally comprising a linker and the bio-distribution modifier optionally comprising a linker, and wherein a linker is optionally interspersed between the amino acid residue linking the chelator optionally comprising a linker and the bio-distribution modifier optionally comprising and the residue of Xaa1,
the chelator is DOTA,
the linker optionally comprised by the chelator is O2Oc or PEG6,
the bio-distribution modifier is AGLU,
the amino acid residue linking the chelator optionally comprising a linker and the bio-distribution modifier optionally comprising is a glu residue,
and the linker optionally interspersed between the amino acid residue linking the chelator optionally comprising a linker and the bio-distribution modifier optionally comprising a linker, and the residue of Xaa1 is APAc, O2Oc or PEG6.

Embodiment 127. The compound of any one of embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125 and 126, wherein Xaa11 is a residue of formula (XIIa), (XIIb) or (XIIc), wherein R^{11b} is C(═O)R^{11c}, wherein R^{11c} is the C-terminal group C-term.

Embodiment 128. The compound of any one of embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126 and 127, preferably the compound of embodiment 127, wherein the C-terminal group C-term is
a) the residue of an amino acid, wherein the amino acid is an α-amino acid,
   optionally comprising a Z group and optionally comprising a linker interspersed between the residue of Xaa11 and the residue of an amino acid,
   wherein if the interspersed linker is absent, the carbonyl group R^{11b} of residue Xaa11 is covalently attached to the α-nitrogen of the residue of an amino acid,
   or wherein if the interspersed linker is present, the carbonyl group in R^{11b} of residue Xaa11 is covalently attached to a nitrogen atom of the linker, a carbonyl group of the linker is covalently attached to the α-nitrogen of the residue of an amino acid,
   and wherein a blocking group AblC is covalently attached to the carbonyl group of the residue of an amino acid, wherein the blocking group AblC is selected from the group consisting of NH₂ and OH, wherein the blocking group AblC preferably is NH₂, or,
b) the C-terminal dipeptide group,
   wherein the residue of the first amino acid is the residue of an α-amino acid and the residue of the second amino acid is the residue of an α-amino acid,
   wherein the residue of the first amino acid, the residue of the second amino acid residue or both the residue of the first and of the second amino acid optionally comprise a Z group,
   and wherein a linker is optionally interspersed between the residue of Xaa11 and the residue of the first amino acid,
   wherein if the interspersed linker is absent, the carbonyl group R^{11b} of Xaa11 is covalently attached to the α-nitrogen of the residue of the first amino acid and the carbonyl group of the residue of the first amino acid is covalently attached to the α-nitrogen of the residue of the second amino acid,
   or wherein if the interspersed linker is present, the carbonyl group R^{1b} of Xaa11 is covalently attached to a nitrogen atom of the linker, a carbonyl group of the linker is covalently attached to the α-nitrogen of the residue of the first amino acid and the carbonyl group of the residue of the first amino acid is covalently attached to the α-nitrogen of the residue of the second amino acid,
   and wherein a blocking group AblC is covalently attached to the carbonyl group of the residue of the second amino acid, wherein the blocking group AblC is selected from the group consisting of NH₂ and OH, wherein the blocking group AblC preferably is NH₂, or
c) a Z group.

Embodiment 129. The compound of any one of embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127 and 128, preferably the compound of any one of embodiments 127 and 128, wherein the C-terminal group C-term is the residue of an α-amino acid, wherein the α-amino acid is selected from
i) an α-amino acid comprising a side chain AaaA, wherein the side chain comprises an amino group, wherein to the amino group a Z group is covalently attached by an amide bond, wherein the Z group is a chelator optionally comprising a linker, wherein the chelator
   a) preferably is selected from the group consisting of DOTA, DOTAGA, DOTAM, DOTP, NOTA, NODAGA, PSC, NODA-MPAA, HBED, TETA, CB-TE2A, DTPA, CHX-A″-DTPA, DFO, Macropa, HOPO, TRAP, THP, DATA, NOPO, NOTP, PCTA, sarcophagine, FSC, NETA, NE3TA, H4octapa, pycup, HYNIC, NxS4×(N4, N2S2, N3S), 99mTc(CO)₃-chelators and their analogs,
   b) more preferably is selected from the group consisting of DOTA, DOTAGA, DOTAM, NOTA, NODAGA, PSC, NODA-MPAA, NOPO, HBED, DTPA, CHX A″-DTPA, CB-TE2A, Macropa, PCTA, N4, and analogs thereof, or
   c) most preferably is selected from the group consisting of DOTA, DOTAGA, DOTAM, NOTA, NODAGA, PSC, NOPO, Macropa, PCTA and analogs thereof,
or
ii) an α-amino acid comprising a side chain AaaB, wherein the side chain comprises an amino group, wherein to the amino group a Z group is covalently attached by an amide bond, wherein the Z group is a bio-distribution modifier optionally comprising a linker, wherein the Z group
   a) preferably is selected from the group consisting of AGLU with Glutar as linker, NTASEROL, GLUSEROL with Glutar as linker, AHOL with Glutar as linker, APOL with Glutar as linker, ABOL with Glutar as linker, APrOL with Glutar as linker, MeO2kDaPEGAc, MeO10kDaPEGAc and MeO36PEG, or
   b) more preferably is MeO36PEG or NTASEROL,
or
iii) an α-amino acid comprising a side chain AaaC, wherein the side chain comprises a COOH group, where to the COOH group a Z group is covalently attached by an amide bond, wherein the Z group is a bio-distribution modifier optionally comprising a linker, wherein the bio-distribution modifier
   a) preferably is selected from the group consisting of AGLU, GLUSEROL, AHOL, APOL, ABOL, and APrOL, or
   b) more preferably is AGLU;
      wherein the blocking group AblC is NH₂,
      and wherein an optional linker is interspersed between Xaa11 and the residue of the α-amino acid, and the linker preferably is APAc or Ttds.

Embodiment 130. The compound of any one of embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128 and 129, preferably the compound of any one of embodiments 127, 128 and 129, more preferably the compound of embodiment 129, wherein the C-terminal group C-term is the residue of an α-amino acid comprising a side chain AaaA, wherein the side chain comprises an amino group, wherein to the amino group a Z group is covalently attached by an amide bond,
wherein the residue of an α-amino acid preferably is a Lys residue or lys residue,
wherein the Z group is a chelator optionally comprising a linker, wherein the chelator preferably is a DOTA,
wherein the optional linker interspersed between the residue of Xaa11 and the residue of the α-amino acid preferably is APAc or Ttds.

Embodiment 131. The compound of any one of embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129 and 130, preferably the compound of any one of embodiments 127, 128 and 129, more preferably the compound of embodiment 129, wherein the C-terminal group C-term is the residue of an α-amino acid comprising a side chain AaaB, wherein the side chain comprises an amino group, wherein to the amino group a Z group is covalently attached by an amide bond,
wherein the residue of an α-amino acid preferably is a residue of an α-amino acid selected from the group comprising Lys, dap and lys,
wherein the Z group is a bio-distribution optionally comprising a linker, wherein the bio-distribution modifier preferably is MeO36PEG or NTASEROL.

Embodiment 132. The compound of any one of embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128 and 129, preferably the compound of any one of embodiments 127, 128 and 129, more preferably the compound of embodiment 129, wherein the C-terminal group C-term is the residue of an α-amino acid comprising a side chain AaaC, wherein the side chain comprises a COOH group, wherein to the COOH group a Z group is covalently attached by an amide bond,
wherein the residue of an α-amino acid preferably is a glu residue,
wherein the Z group is a bio-distribution modifier optionally comprising a linker, wherein the bio-distribution modifier preferably is AGLU,
wherein the optional linker interspersed between the residue of Xaa11 and the residue of an α-amino acid preferably is APAc or Ttds.

Embodiment 133. The compound of any one of embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129 and 132, preferably the compound of any one of embodiments 127, 128, 129 and 132, more preferably the compound of embodiment 132, wherein the residue of an α-amino acid is a glu residue, the bio-distribution modifier is AGLU and the linker interspersed between the residue of Xaa11 and the glu residue is APAc.

Embodiment 134. The compound of any one of embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127 and 128, preferably the compound of any one of embodiments 127 and 128, wherein the C-terminal group C-term is the C-terminal dipeptide group,
wherein the first of the two amino acid residues is a residue of an α-amino acid comprising a side chain AaaB,
   wherein the side chain comprises an amino group,
   wherein to the amino group a Z group is covalently attached by an amide bond,
   wherein the Z group is a bio-distribution modifier optionally comprising a linker,
   wherein the Z group
      a) preferably is selected from the group consisting of AGLU with Glutar as linker, NTASEROL, GLUSEROL with Glutar as linker, AHOL with Glutar as linker, APOL with Glutar as linker, ABOL with Glutar as linker, APrOL with Glutar as linker, MeO2kDaPEGAc, MeO10kDaPEGAc and MeO36PEG, or
      b) more preferably is MeO2kDaPEGAc,
and wherein the second of the two amino acid residues is a residues of an α-amino acid comprising a side chain AaaA,
   wherein the side chain comprises an amino group,
   wherein to the amino group a Z group is covalently attached by an amide bond,
   wherein the Z group is a chelator optionally comprising a linker,
   wherein the chelator
      a) preferably is selected from the group consisting of DOTA, DOTAGA, DOTAM, DOTP, NOTA, NODAGA, PSC, NODA-MPAA, HBED, TETA, CB-TE2A, DTPA, CHX-A″-DTPA, DFO, Macropa, HOPO, TRAP, THP, DATA, NOPO, NOTP, PCTA, sarcophagine, FSC, NETA, NE3TA, H4octapa, pycup, HYNIC, NxS4×(N4, N2S2, N3S), 99mTc(CO)₃-chelators and their analogs,
      b) more preferably is selected from the group consisting of DOTA, DOTAGA, DOTAM, NOTA, NODAGA, PSC, NODA-MPAA, NOPO, HBED, DTPA, CHX A″-DTPA, CB-TE2A, Macropa, PCTA, N4, and analogs thereof, or
      c) most preferably is selected from the group consisting of DOTA, DOTAGA, DOTAM, NOTA, NODAGA, PSC, NOPO, Macropa, PCTA and analogs thereof,
         wherein the blocking group AblC is NH₂,
         and wherein an optional linker is interspersed between the residue of Xaa11 and the first of the two amino acid residues of the C-terminal dipeptide group, and wherein the linker preferably is APAc or Ttds.

Embodiment 135. The compound of any one of embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128 and 134, preferably the compound of any one of embodiments 127, 128 and 134, more preferably the compound of embodiment 134,
wherein the first of the two amino acid residues is a residue of an α-amino acid comprising a side chain AaaB,
   wherein the side chain comprises an amino group,
   wherein to the amino group a Z group is covalently attached by an amide bond,
   wherein the residue of the first amino acid preferably is a Lys residue,
   wherein the Z group attached to the residue of the first of the two amino acid residues is a bio-distribution modifier optionally comprising a linker,
   wherein the bio-distribution modifier preferably is a MeO2kDaPEGAc residue,
and wherein the second of the two amino acid residues is a residue of an α-amino acid a side chain AaaA,
   wherein the side chain comprises an amino group,
   wherein to the amino group a Z group is covalently attached by an amide bond,
   wherein residue of the second amino acid preferably is a Lys residue
   wherein the Z group attached to the residue of the second of the two amino acid residues is a chelator optionally comprising a linker,
   wherein the chelator preferably is a DOTA.

Embodiment 136. The compound of any one of embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127 and 128, preferably the compound of any one of embodiments 127 and 128, wherein the C-terminal group C-term is the C-terminal dipeptide group,
wherein the first of the two amino acid residues is a residue of an α-amino acid comprising a side chain AaaC,
   wherein the side chain comprises an COOH group,
   wherein to the COOH group a Z group is covalently attached by an amide bond,
   wherein the residue of the first amino acid preferably is a glu residue,
   wherein the Z group is a bio-distribution modifier optionally comprising a linker,
   wherein the bio-distribution modifier
      a) preferably is selected from the group consisting of AGLU, GLUSEROL, AHOL, APOL, ABOL, and APrOL, or
      b) more preferably is AGLU,
and wherein the second of the two amino acid residues is a residue of an α-amino acid comprising a side chain AaaA,
   wherein the side chain comprises an amino group,
   wherein to the amino group a Z group is covalently attached by an amide bond,
   wherein the residue of the first amino acid preferably is a Lys residue,
   wherein the Z group is a chelator optionally comprising a linker, wherein the chelator
      a) preferably is selected from the group consisting of DOTA, DOTAGA, DOTAM, DOTP, NOTA, NODAGA, PSC, NODA-MPAA, HBED, TETA, CB-TE2A, DTPA, CHX-A″-DTPA, DFO, Macropa, HOPO, TRAP, THP, DATA, NOPO, NOTP, PCTA, sarcophagine, FSC, NETA, NE3TA, H4octapa, pycup, HYNIC, NxS4×(N4, N2S2, N3S), 99mTc(CO)₃-chelators and their analogs,
      b) more preferably is selected from the group consisting of DOTA, DOTAGA, DOTAM, NOTA, NODAGA, PSC, NODA-MPAA, NOPO, HBED, DTPA, CHX A″-DTPA, CB-TE2A, Macropa, PCTA, N4, and analogs thereof, or
      c) most preferably is selected from the group consisting of DOTA, DOTAGA, DOTAM, NOTA, NODAGA, PSC, NOPO, Macropa, PCTA and analogs thereof,
         and wherein an optional linker is interspersed between the residue of Xaa11 and the first of the two amino acid residues of the C-terminal dipeptide group, and wherein the linker preferably is APAc or Ttds.

Embodiment 137. The compound of any one of embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128 and 136, preferably the compound of any one of embodiments 127, 128 and 136, more preferably the compound of embodiment 136,
wherein the first of the two amino acid residues is a residue of an α-amino acid comprising a side chain AaaC,
   wherein the side chain comprises a COOH group,
   wherein to the COOH group a Z group is covalently attached by an amide bond,
   wherein the residue of the first amino acid preferably is a glu residue,
   wherein the Z group is a bio-distribution modifier optionally comprising a linker,
   wherein the bio-distribution modifier preferably is an AGLU residue,
and wherein the second of the two amino acid residues is a residue of an α-amino acid comprising a side chain AaaA,
   wherein the side chain comprises an amino group,
   wherein to the amino group a Z group is covalently attached by an amide bond,
   wherein the residue of the first amino acid preferably is a Lys residue or lys residue,
   wherein the Z group is a chelator optionally comprising a linker,
   wherein the chelator preferably is a DOTA.

Embodiment 138. The compound of any one of embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127 and 128, preferably the compound of any one of embodiments 127 and 128, wherein the C-terminal group C-term is a Z group,
wherein if the Z group is a chelator optionally comprising a linker, the chelator preferably is DOTA and the optional linker preferably is selected from the group consisting of PP and Ape, and
wherein if the Z group is a bio-distribution modifier optionally comprising a linker, the bio-distribution modifier preferably is GLUSEROL and the optional linker preferably is APAc.

Embodiment 139. The compound of any one of embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128 and 138, preferably the compound of any one of embodiments 127, 128 and 138, more preferably the compound of embodiment 138, wherein Xaa11 is a residue of formula (XIIa), wherein R^{11b} is C(═O)R^{11c}, wherein R^{11c} is C-term, the C-terminal group C-term is a Z group, wherein the Z group is a bio-distribution modifier optionally comprising a linker, the bio-distribution modifier preferably is GLUSEROL and the optional linker preferably is APAc.

Embodiment 140. The compound of any one of embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 138 and 139, wherein the compound comprises one single Z group.

Embodiment 141. The compound of any one of embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 138, 139 and 140, preferably the compound of embodiment 140, wherein the Z group
a) is covalently attached to or is part of substituent R^{4d}, under the proviso that Xaa4 is a structure of formula (Va), (Vd) or (Ve),
b) is covalently attached to or is part of substituent R^{4h}, under the proviso that Xaa4 is a structure of formula (Vb) or (Vf),
c) is covalently attached to or is part of substituent R^{5d}, under the proviso that Xaa5 is a structure of formula (VIa),
d) is covalently attached to or is part of substituent R^{8a}, under the proviso that Xaa8 is a structure of formula (XIa), (IXd) or (IXe),
e) is covalently attached to or is part of substituent R^{8e}, under the proviso that Xaa8 is a structure of formula (XIc),
f) is covalently attached to or is part of substituent R^{Yd}, under the proviso that Yc is a structure of formula (XIIIh), comprising a residue R^{Yc}, wherein to R^{Yc} a residue R^{Yd} is covalently attached, wherein R^{Yd} is a structure of formula (XIIIc), (XIIId) or (XIIIe),
g) is covalently attached to the residue of an α-amino acid of the N-terminal modification group A,
h) is covalently attached to one of the two residues of the N-terminal dipeptide group,
i) is covalently attached to the residue of an α-amino acid of the C-terminal group C-term,
j) is part of or forms the N-terminal modification group A,
or
k) is the C-terminal group C-term.

Embodiment 142. The compound of any one of embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 138, 139, 140 and 141, preferably the compound of embodiment 141, wherein the Z group
a) is covalently attached to or is part of substituent R^{8a}, under the proviso that Xaa8 is a structure of formula (XIa), (IXd) or (IXe),
b) is covalently attached to or is part of substituent R^{8e}, under the proviso that Xaa8 is a structure of formula (XIc),
c) is covalently attached to the residue of an α-amino acid of the C-terminal group C-term,
d is part of or forms the N-terminal modification group A,
or
e) is the C-terminal group C-term.

Embodiment 143. The compound of any one of embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 138 and 139, wherein the compound comprises two or more Z groups.

Embodiment 144. The compound of any one of embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 138, 139 and 143, preferably the compound of embodiment 143, wherein each of the two or more Z groups
a) is covalently attached to or is part of substituent R^{4d}, under the proviso that Xaa4 is a structure of formula (Va), (Vd) or (Ve),
b) is covalently attached to or is part of substituent R^{4h}, under the proviso that Xaa4 is a structure of formula (Vb) or (Vf),
c) is covalently attached to or is part of substituent R^{5d}, under the proviso that Xaa5 is a structure of formula (VIa),
d) is covalently attached to or is part of substituent R^{8a}, under the proviso that Xaa8 is a structure of formula (XIa), (IXd) or (IXe),
e) is covalently attached to or is part of substituent R^{8e}, under the proviso that Xaa8 is a structure of formula (XIc),
f) is covalently attached to or is part of substituent R^{Yd}, under the proviso that Yc is a structure of formula (XIIIh), comprising a residue R^{Yc}, wherein to R^{Yc} a residue R^{Yd} is covalently attached, wherein R^{Yd} is a structure of formula (XIIIc), (XIIId) or (XIIIe),
g) is covalently attached to the residue of the α-amino acid of the N-terminal modification group A,
h) is covalently attached to one of the two residues of the N-terminal dipeptide group,
i) is covalently attached to the residue of an α-amino acid of the C-terminal group C-term,
j) is covalently attached to one of the two residues of the C-terminal dipeptide group,
k) is part of or forms the N-terminal modification group A,
or
l) is the C-terminal group C-term.

Embodiment 145. The compound of any one of embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 138, 139, 143 and 144, preferably the compound of embodiment 144, wherein each of the two or more Z groups
a) is covalently attached to or is part of substituent R^{4d}, under the proviso that Xaa4 is a structure of formula (Va), (Vd) or (Ve),
b) is covalently attached to or is part of substituent R^{8a}, under the proviso that Xaa8 is a structure of formula (XIa), (IXd) or (IXe),
c) is covalently attached to or is part of substituent R^{8e}, under the proviso that Xaa8 is a structure of formula (XIc),
d) is covalently attached to the residue of an α-amino acid of the C-terminal group C-term,
e) is covalently attached to one of the two residues of the C-terminal dipeptide group,
f) is part of or forms the N-terminal modification group A,
or
g) is the C-terminal group C-term.

Embodiment 146. The compound of any one of embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 138, 139, 143, 144 and 145, preferably the compound of any one of embodiments 144 and 145, more preferably the compound of embodiment 145, wherein the compound comprises two to four Z groups, preferably two to three Z groups, more preferably three Z groups, most preferably one of the three Z groups is a chelator optionally comprising a linker and the other two of the three Z groups are bio-distribution modifiers each optionally comprising a linker.

Embodiment 147. The compound of any one of embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 138, 139, 143, 144, 145 and 146, preferably the compound of any one of embodiments 143, 144, 145 and 146, more preferably the compound of embodiment 146, wherein the compound comprises two Z groups, wherein one of the two Z groups is a chelator optionally comprising a linker and the other of the two Z groups is a bio-distribution modifier optionally comprising a linker, and wherein the chelator optionally comprising a linker preferably
a) is covalently attached to or is part of substituent R^{8a}, under the proviso that Xaa8 is a structure of formula (XIa), (IXd) or (IXe),
b) is covalently attached to or is part of substituent R^{8e}, under the proviso that Xaa8 is a structure of formula (XIc),
c) is covalently attached to the residue of an α-amino acid of the C-terminal group C-term,
or
d) is covalently attached to one of the two residues of the C-terminal dipeptide group,
   and wherein the bio-distribution modifier optionally comprising a linker
a) is covalently attached to or is part of substituent R^{4d}, under the proviso that Xaa4 is a structure of formula (Va), (Vd) or (Ve),
b) is covalently attached to the residue of an α-amino acid of the C-terminal group C-term
c) is covalently attached to one of the two residues of the C-terminal dipeptide group,
or
d) is part of or forms the N-terminal modification group A.

Embodiment 148. The compound of any one of embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 138, 139, 143, 144, 145, 146 and 147, preferably the compound of any one of embodiments 143, 144, 145, 146 and 147, more preferably the compound of embodiment 147, wherein the chelator optionally comprising a linker preferably
is covalently attached to or is part of substituent R^{8a}, under the proviso that residue
Xaa8 is a structure of formula (XIa), (IXd) or (IXe),
   and wherein the bio-distribution modifier optionally comprising a linker preferably
a) is covalently attached to the residue of an α-amino acid of the C-terminal group C-term
or
b) is part of or forms the N-terminal modification group A.

Embodiment 149. The compound of any one of embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 138, 139, 143, 144, 145, 146 and 147, preferably the compound of any one of embodiments 143, 144, 145, 146 and 147, more preferably the compound of embodiment 147, wherein the chelator optionally comprising a linker preferably
is covalently attached to one of the two residues of the C-terminal dipeptide group and wherein the bio-distribution modifier optionally comprising a linker
is covalently attached to other one of the two residues of the C-terminal dipeptide group.

Embodiment 150. The compound of any one of embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 138, 139, 143, 144, 145 and 146, preferably the compound of any one of embodiments 143, 144, 145 and 146, more preferably the compound of embodiment 146, wherein the compound comprises two Z groups, wherein one of the two Z group is a combination of a chelator optionally comprising a linker, a bio-distribution modifier optionally comprising a linker, and an amino acid residue linking the chelator optionally comprising a linker and the bio-distribution modifier optionally comprising a linker, and the other of the two Z groups is a bio-distribution modifier optionally comprising a linker, and wherein the combination of chelator optionally comprising a linker and bio-distribution modifier optionally comprising a linker
forms the N-terminal modification group A,
   and wherein the bio-distribution modifier optionally comprising a linker preferably
a) is covalently attached to or is part of substituent R^{8a}, under the proviso that Xaa8 is a structure of formula (XIa), (IXd) or (IXe),
b) is covalently attached to or is part of substituent R^{8e}, under the proviso that Xaa8 is a structure of formula (XIc),
c) is covalently attached to the residue of an α-amino acid of the C-terminal group C-term.

Embodiment 151. The compound of any one of embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 138, 139, 143, 144, 145 and 146, preferably the compound of any one of embodiments 143, 144, 145 and 146, more preferably the compound of embodiment 146, wherein the compound comprises three Z groups, wherein one of the three Z groups is a chelator optionally comprising a linker, and the other two of the three Z groups are bio-distribution modifiers optionally comprising a linker, wherein the chelator optionally comprising a linker preferably
a) is covalently attached to or is part of substituent R^{8a}, under the proviso that Xaa8 is a structure of formula (XIa), (IXd) or (IXe),
b) is covalently attached to or is part of substituent R^{8e}, under the proviso that residue Xaa8 is a structure of formula (XIc),
c) is covalently attached to the residue of an α-amino acid of the C-terminal group C-term,
or
d) is part of or forms the N-terminal modification group A,
   and wherein either of the two the bio-distribution modifiers optionally comprising a linker
a) is covalently attached to or is part of substituent R^{8a}, under the proviso that residue Xaa8 is a structure of formulae (XIa), (IXd) or (IXe),
b) is covalently attached to or is part of substituent R^{8e}, under the proviso that residue Xaa8 is a structure of formula (XIc),
c) is covalently attached to the residue of an α-amino acid of the C-terminal group C-term,
or
d) is part of or forms the N-terminal modification group A.

Embodiment 152. The compound of any one of embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 138, 139, 143, 144, 145, 146 and 151, preferably the compound of any one of embodiments 143, 144, 145, 146 and 151, more preferably the compound of embodiment 151, wherein the chelator optionally comprising a linker
is part of or forms the N-terminal modification group A,
   and wherein one of the two bio-distribution modifiers optionally comprising a linker
a) is covalently attached to or is part of substituent R^{8a}, under the proviso that residue Xaa8 is a structure of formula (XIa), (IXd) or (IXe),
or
b) is covalently attached to or is part of substituent R^{8e}, under the proviso that residue Xaa8 is a structure of formula (XIc),
   and wherein the other of the two bio-distribution modifiers optionally comprising a linker
is covalently attached to the residue of an α-amino acid of the C-terminal group C-term.

Embodiment 153. The compound of any one of embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 138, 139, 143, 144, 145, 146 and 151, preferably the compound of any one of embodiments 143, 144, 145, 146 and 151, more preferably the compound of embodiment 151, wherein the chelator optionally comprising a linker
a) is covalently attached to or is part of substituent R^{8a}, under the proviso that residue Xaa8 is a structure of formula (XIa), (IXd) or (IXe),
or
b) is covalently attached to or is part of substituent R^{8e}, under the proviso that residue Xaa8 is a structure of formula (XIc),
   and wherein one of the two bio-distribution modifiers each optionally comprising a linker
is part of or forms the N-terminal modification group A,
   and wherein the other of the two bio-distribution modifiers each optionally comprising a linker
is covalently attached to the residue of an α-amino acids of the C-terminal group C-term.

Embodiment 154. The compound of any one of embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 138, 139, 143, 144, 145, 146 and 151, preferably the compound of any one of embodiments 143, 144, 145, 146 and 151, more preferably the compound of embodiment 151, wherein the chelator optionally comprising a linker
is covalently attached to the residue of an α-amino acid of the C-terminal group C-term
   and wherein one of the two bio-distribution modifiers each optionally comprising a linker
is part of or forms the N-terminal modification group A,
   and wherein the other of the two bio-distribution modifiers each optionally comprising a linker
a) is covalently attached to or is part of substituent R^{8a}, under the proviso that Xaa8 is a structure of formula (XIa), (IXd) or (IXe),
or
b) is covalently attached to or is part of substituent R^{8e}, under the proviso that residue Xaa8 is a structure of formula (XIc).

Embodiment 155. The compound of any one of embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 138, 139, 143, 144, 145 and 146, preferably the compound of any one of embodiments 143, 144, 145 and 146, more preferably the compound of embodiment 146, wherein the compound comprises three Z groups, wherein one of the three Z group is a combination of a chelator optionally comprising a linker, a bio-distribution modifier optionally comprising a linker, and an amino acid residue linking the chelator optionally comprising a linker and the bio-distribution modifier optionally comprising a linker and the other two of the three Z groups are bio-distribution modifiers each optionally comprising a linker, and wherein the combination of chelator optionally comprising a linker and bio-distribution modifier optionally comprising a linker
forms the N-terminal modification group A,
   and wherein one of the two bio-distribution modifiers optionally comprising a linker
a) is covalently attached to or is part of substituent R^{8a}, under the proviso that residue Xaa8 is a structure of formula (XIa), (IXd) or (IXe),
or
b) is covalently attached to or is part of substituent R^{8e}, under the proviso that residue Xaa8 is a structure of formula (XIc),
   and wherein the other one of the two bio-distribution modifiers each optionally comprising a linker
is covalently attached to the residue of an α-amino acid of the C-terminal group C-term.

Embodiment 156. The compound of any one of embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 138, 139, 143, 144, 145 and 146, preferably the compound of any one of embodiments 143, 144, 145 and 146, more preferably the compound of embodiment 146, wherein the compound comprises three Z groups, wherein one of the three Z group is a combination of a chelator optionally comprising a linker, a bio-distribution modifier optionally comprising a linker, and an amino acid residue linking the chelator optionally comprising a linker and the bio-distribution modifier optionally comprising a linker and the other two of the three Z groups are bio-distribution modifiers optionally comprising a linker, and wherein the combination of a chelator optionally comprising a linker and a bio-distribution modifier optionally comprising a linker
a) is covalently attached to or is part of substituent R^{8a}, under the proviso that residue Xaa8 is a structure of formula (XIa), (IXd) or (IXe),
or
b) is covalently attached to or is part of substituent R^{8e}, under the proviso that residue Xaa8 is a structure of formula (XIc),
   and wherein one of the two bio-distribution modifiers each optionally comprising a linker
is part of or forms the N-terminal modification group A,
   and wherein the other one of the two bio-distribution modifiers each optionally comprising a linker
is covalently attached to the residue of an α-amino acid of the C-terminal group C-term.

Embodiment 157. The compound of any one of embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 138, 139, 143, 144, 145 and 146, preferably the compound of any one of embodiments 143, 144, 145 and 146, more preferably the compound of embodiment 146, wherein the compound comprises four Z groups, wherein one of the four Z groups is a chelator optionally comprising a linker, and the other three of the four Z groups are bio-distribution modifiers each optionally comprising a linker, wherein the chelator optionally comprising a linker preferably
is covalently attached to one of the two residues of the C-terminal dipeptide group, a first of the three bio-distribution modifiers optionally comprising a linker
is part of or forms the N-terminal modification group A,
   a second of the three bio-distribution modifiers optionally comprising a linker
a) is covalently attached to or is part of substituent R^{8a}, under the proviso that residue Xaa8 is a structure of formula (XIa), (IXd) or (IXe),
or
b) is covalently attached to or is part of substituent R^{8e}, under the proviso that residue Xaa8 is a structure of formula (XIc),
   and a third of the three bio-distribution modifiers optionally comprising a linker
is covalently attached to other one of the two residues of the amino acids of the C-terminal dipeptide group.

Embodiment 158. The compound of any one of embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 138, 139, 143, 144, 145 and 146, preferably the compound of any one of embodiments 143, 144, 145 and 146, more preferably the compound of embodiment 146, wherein the compound comprises four Z groups, wherein each of the four Z groups is a chelator optionally comprising a linker, and wherein a first of the four chelators each optionally comprising a linker
is covalently attached to or is part of substituent R^{4d}, under the proviso that residue Xaa4 is a structure of formulae (Va), (Vd) or (Ve),
   and a second of the four chelators each optionally comprising a linker
a) is covalently attached to or is part of substituent R^{8a}, under the proviso that residue Xaa8 is a structure of formula (XIa), (IXd) or (IXe),
or
b) is covalently attached to or is part of substituent R^{8e}, under the proviso that residue Xaa8 is a structure of formula (XIc),
   a third of the four chelators each optionally comprising a linker
is part of or forms the N-terminal modification group A,
   and a forth of the four chelators each optionally comprising a linker
is covalently attached to the residue of an α-amino acid of the C-terminal group C-term.

Embodiment 159. The compound of any one of embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 138, 139, 143, 144, 145 and 146, preferably the compound of any one of embodiments 143, 144, 145 and 146, more preferably the compound of embodiment 146, wherein the compound comprises three Z groups, wherein each of the three Z groups is a chelator optionally comprising a linker, and wherein a first of the three chelators each optionally comprising a linker
a) is covalently attached to or is part of substituent R^{8a}, under the proviso that residue Xaa8 is a structure of formula (XIa), (IXd) or (IXe),
or
b) is covalently attached to or is part of substituent R^{8e}, under the proviso that residue Xaa8 is a structure of formula (XIc),
   wherein a second of the three chelators each optionally comprising a linker
is part of or forms the N-terminal modification group A,
   and a third of the three chelators each optionally comprising a linker
is covalently attached to the residue of an α-amino acid of the C-terminal group C-term.

Embodiment 160. The compound of any one of embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 138, 139, 143, 144, 145 and 146, preferably the compound of any one of embodiments 143, 144, 145 and 146, more preferably the compound of embodiment 146, wherein the compound comprises two Z groups, wherein each of the two Z groups is a chelator optionally comprising a linker, and wherein a first of the two chelators each optionally comprising a linker
a) is covalently attached to or is part of substituent R^{8a}, under the proviso that residue Xaa8 is a structure of formulae (XIa), (IXd) or (IXe),
or
b) is covalently attached to or is part of substituent R^{8e}, under the proviso that residue Xaa8 is a structure of formula (XIc),
   and a second of the two chelators each optionally comprising a linker
a) is part of or forms the N-terminal modification group A,
or
b) is covalently attached to the residue of an α-amino acid of the C-terminal group C-term.

Embodiment 161. The compound of any one of embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 138, 139, 143, 144, 145 and 146, preferably the compound of any one of embodiments 143, 144, 145 and 146, more preferably the compound of embodiment 146, more preferably the compound of embodiment 146, wherein the compound comprises two Z groups, wherein each of the two Z groups is a chelator optionally comprising a linker, and wherein a first of the two chelators each optionally comprising a linker
is part of or forms the N-terminal modification group A
   and a second of the two chelators each optionally comprising a linker
is covalently attached to the residue of an α-amino acid of the C-terminal group C-term.

Embodiment 162. The compound of any one of embodiments 1, 2, 3, 4, 5, 6, 7 and 8, wherein
Xaa1 is Asp, Asn, Glu, Gln, Succinyl, Glutar or Cya,
Xaa2 is Trp, Hyw, 5Fw, 6Clw or 7Fw,
Xaa3 is 5Clw, 5Brw, 5Fw, Trp, 1Ni, Bta, 6Fw or Trp,
Xaa4 is Tap, Hyp, 4Tfp, Pro, H3p, Eaz, Oxa, Dtc, Nmg, Nleu, Nlys, Nphe or Nglu,
Xaa5 is Glu, glu, Nme, Gln, Asp, Asn or Hse,
Xaa6 is leu, Leu, Npg, Val or Ile,
Xaa7 is Cys or Hey,
Xaa8 is Apc(DOTA), Thp, Apc, Aib, Deg, Ams, Amk(DOTA), Egz, Eca, or ala,
Xaa9 is Leu, Hle, Ile, Phe, Glu, Arg, Ser or Trp,
Xaa10 is Ile, Leu, Val or Tle,
Xaa11 is Cys, Hey or AET, and
Yc is 3MeBn or 3Lut.

Embodiment 163. The compound of any one of embodiments 1, 2, 3, 4, 5, 6, 7 and 8, wherein
Xaa1 is Asp, Glu or Succinyl,
Xaa2 is Trp or Hyw,
Xaa3 is 5Clw or 5Brw,
Xaa4 is Tap, Hyp, or Pro,
Xaa5 is Glu,
Xaa6 is leu or Leu,
Xaa7 is Cys,
Xaa8 is Apc(DOTA) or Thp,
Xaa9 is Leu,
Xaa10 is Ile
Xaa11 is Cys and
Yc is 3Lut (SEQ ID NO: 3).

Embodiment 164. The compound of any one of embodiments 1, 2, 3, 4, 5, 6, 7 and 8, wherein
Xaa1 is Asp,
Xaa2 is Trp,
Xaa3 is 5Clw,
Xaa4 is Tap or Hyp,
Xaa5 is Glu,
Xaa6 is leu,
Xaa7 is Cys,
Xaa8 is Apc(DOTA),
Xaa9 is Leu,
Xaa10 is Ile
Xaa11 is Cys and
Yc is 3Lut (SEQ ID NO: 4).

Embodiment 165. The compound of any one of embodiments 1, 2, 3, 4, 5, 6, 7 and 8, wherein the N-terminal modification group A is
a) a Z group, wherein a linker is optionally interspersed between the Z group and Xaa1,
   wherein
      the Z group is a chelator optionally comprising a linker,
      or the Z group is a bio-distribution modifier optionally comprising a linker,
      or the Z group is a combination of a chelator optionally comprising a linker, a bio-distribution modifier optionally comprising a linker, and an amino acid residue linking the chelator optionally comprising a linker and the bio-distribution modifier optionally comprising a linker
b) a blocking group AblN, wherein the blocking group AblN is selected from the group consisting of acetyl and a structure of formula (AblN-Ia),

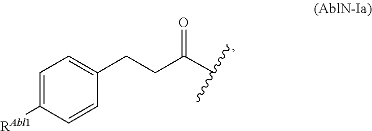

wherein
   R^{Abl1} is selected from the group consisting of H, OH and a halogen, wherein the halogen is preferably selected from the group consisting of F and Cl, preferably R^{Abl1} is OH
preferably AblN is selected from the group consisting of Ac, 4OHPhp, 4FPhp, 4ClPhp and Php,
c) a residue of an α-amino acid, wherein the residue of an α-amino acid
   optionally comprises a Z group covalently attached to the side chain of the residue of the α-amino acid and
   optionally comprises a Z group is covalently attached to the α-amino group of the residue of the α-amino acid,
   or wherein the residue of an α-amino acid optionally comprises a blocking group AblN covalently attached to the α-amino group of the residue of the α-amino acid, preferably the blocking group AblN is acetyl,
   wherein the residue of an α-amino acid preferably is a Tyr residue, wherein optionally a Z group is covalently attached to the α-amino group of the Tyr residue, wherein the Z group is a chelator optionally comprising a linker, wherein the chelator preferably is DOTA and the linker preferably is Ttds,
d) the N-terminal dipeptide group,
   wherein the residue of the first amino acid, the residue of the second amino acid or both of the residue of first and the residue of the second amino acid residue optionally comprise a Z group,
   wherein, optionally, a blocking group AblN is covalently attached to the α-amino group of the residue of the first amino acid,
   and wherein a linker is optionally interspersed between the residue of the second amino acid and the residue of Xaa1,
   wherein if the interspersed linker is absent, the carbonyl group of the residue of the first amino acid is covalently attached to the α-nitrogen of the residue of the second amino acid and the carbonyl group of the residue of the second amino acid is covalently attached to the nitrogen atom in R^{1a} or the α-nitrogen atom of residue Xaa1,
   or wherein if the interspersed linker is present, the carbonyl group of the residue of the first amino acid is covalently attached to the α-nitrogen of the residue of the second and the carbonyl group of the residue of the second amino acid is covalently attached to a nitrogen atom of the linker and a carbonyl group of the linker is covalently attached to the nitrogen atom in R^{1a} or the α-nitrogen atom of residue Xaa1,
   wherein the residue of the first amino acid residue is a Met residue and the residue of the second amino acid is selected from the group comprising Ala, Tyr and tyr.

Embodiment 166. The compound of any one of embodiments 1, 2, 3, 4, 5, 6, 7, 8, 162 and 165, preferably the compound of any one of embodiments 162 and 165, wherein under the proviso that Xaa1 is selected from the group consisting of Asp, Asn, Glu, Gln and Cya, the N-terminal modification group A is
a) selected from the group consisting of AGLU-Glutar, NTASEROL, AGLU-Glutar-APAc, DOTA, DOTA-Ttds, DOTA-APAc, MeO36PEG-APAc, MeO2kDaPEGAc, DOTA-glu(AGLU), DOTA-glu(AGLU)-APAc, DOTA-O2Oc-glu(AGLU), DOTA-glu(AGLU)-O2Oc, DOTA-PEG6-glu(AGLU), and DOTA-glu(AGLU)-PEG6, preferably is AGLU-Glutar or NTASEROL, or
b) selected from the group consisting of Ac, Php, 4FPhp, 4ClPhp and 4OHPhp, preferably is 4OHPhp, or
c) DOTA-Ttds-Tyr or Ac-Tyr, or
d) Met-Tyr, Met-tyr or Met-Ala.

Embodiment 167. The compound of any one of embodiments 1, 2, 3, 4, 5, 6, 7, 8, 162, 165 and 166, preferably the compound of any one of embodiments 162, 165 and 166, more preferably the compound of embodiment 166, wherein under the proviso that Xaa1 is selected from the group consisting of Asp, Asn, Glu, Gln and Cya, the N-terminal modification group A is selected from the group consisting of AGLU-Glutar, NTASEROL, AGLU-Glutar-APAc, DOTA, DOTA-Ttds, DOTA-APAc, MeO36PEG-APAc, MeO2kDaPEGAc, DOTA-glu(AGLU), DOTA-glu(AGLU)-APAc, DOTA-O2Oc-glu(AGLU), DOTA-glu(AGLU)-O2Oc, DOTA-PEG6-glu(AGLU), and DOTA-glu(AGLU)-PEG6, preferably is AGLU-Glutar or NTASEROL.

Embodiment 168. The compound of any one of embodiments 1, 2, 3, 4, 5, 6, 7, 8, 162 and 165 to 167, preferably the compound of any one of embodiments 162 and 165 to 167, more preferably the compound of embodiment 167, wherein under the proviso that Xaa1 is selected from the group consisting of Asp, Asn, Glu, Gln and Cya, the N-terminal modification group A is selected from the group consisting of AGLU-Glutar, NTASEROL, DOTA-glu(AGLU)-APAc, DOTA and DOTA-Ttds, preferably is AGLU-Glutar or NTASEROL.

Embodiment 169. The compound of any one of embodiments 1, 2, 3, 4, 5, 6, 7, 8, 163 and 165, preferably the compound of any one of embodiments 163 and 165, wherein under the proviso that Xaa1 is selected from the group consisting of Asp and Glu, the N-terminal modification group A is
a) selected from the group consisting of AGLU-Glutar, NTASEROL, AGLU-Glutar-APAc, DOTA, DOTA-Ttds, DOTA-APAc, MeO36PEG-APAc, MeO2kDaPEGAc, DOTA-glu(AGLU), DOTA-glu(AGLU)-APAc, DOTA-O2Oc-glu(AGLU), DOTAglu(AGLU)-O2Oc, DOTA-PEG6-glu(AGLU), and DOTA-glu(AGLU)-PEG6, preferably is AGLU-Glutar or NTASEROL, or
b) selected from the group consisting of Ac, Php, 4FPhp, 4ClPhp and 4OHPhp, preferably is 4OHPhp, or
c) DOTA-Ttds-Tyr or Ac-Tyr, or
d) Met-Tyr, Met-tyr or Met-Ala.

Embodiment 170. The compound of any one of embodiments 1, 2, 3, 4, 5, 6, 7, 8, 163, 165 and 169, preferably the compound of any one of embodiments 163, 165 and 169, more preferably the compound of embodiment 169, wherein under the proviso that Xaa1 is selected from the group consisting of Asp and Glu, the N-terminal modification group A is selected from the group consisting of AGLU-Glutar, NTASEROL, AGLU-Glutar-APAc, DOTA, DOTA-Ttds, DOTA-APAc, MeO36PEG-APAc, MeO2kDaPEGAc, DOTA-glu(AGLU), DOTA-glu(AGLU)-APAc, DOTA-O2Oc-glu(AGLU), DOTA-glu(AGLU)-O2Oc, DOTA-PEG6-glu(AGLU), and DOTA-glu(AGLU)-PEG6, preferably is AGLU-Glutar or NTASEROL.

Embodiment 171. The compound of any one of embodiments 1, 2, 3, 4, 5, 6, 7, 8, 163, 165, 169 and 170, preferably the compound of any one of embodiments 163, 165, 169 and 170, more preferably the compound of embodiment 170, wherein under the proviso that Xaa1 is selected from the group consisting of Asp and Glu, the N-terminal modification group A is selected from the group consisting of AGLU-Glutar, NTASEROL, DOTA-glu(AGLU)-APAc, DOTA and DOTA-Ttds, preferably is AGLU-Glutar or NTASEROL.

Embodiment 172. The compound of any one of embodiments 1, 2, 3, 4, 5, 6, 7, 8, 164 and 165, preferably the compound of any one of embodiments 164 and 165, wherein the N-terminal modification group A is
a) selected from the group consisting of AGLU-Glutar, NTASEROL, AGLU-Glutar-APAc, DOTA, DOTA-Ttds, DOTA-APAc, MeO36PEG-APAc, MeO2kDaPEGAc, DOTA-glu(AGLU), DOTA-glu(AGLU)-APAc, DOTA-O2Oc-glu(AGLU), DOTA-glu(AGLU)-O2Oc, DOTA-PEG6-glu(AGLU), and DOTA-glu(AGLU)-PEG6, preferably is AGLU-Glutar or NTASEROL, or
b) selected from the group consisting of Ac, Php, 4FPhp, 4ClPhp and 4OHPhp, preferably is 4OHPhp, or
c) DOTA-Ttds-Tyr or Ac-Tyr, or
d) Met-Tyr, Met-tyr or Met-Ala.

Embodiment 173. The compound of any one of embodiments 1, 2, 3, 4, 5, 6, 7, 8, 164, 165 and 172, preferably the compound of any one of embodiments 164, 165 and 172, more preferably the compound of embodiment 172, wherein the N-terminal modification group A is selected from the group consisting of AGLU-Glutar, NTASEROL, AGLU-Glutar-APAc, DOTA, DOTA-Ttds, DOTA-APAc, MeO36PEG-APAc, MeO2kDaPEGAc, DOTA-glu(AGLU), DOTA-glu(AGLU)-APAc, DOTA-O2Oc-glu(AGLU), DOTA-glu(AGLU)-O2Oc, DOTA-PEG6-glu(AGLU), and DOTA-glu(AGLU)-PEG6, preferably is AGLU-Glutar or NTASEROL.

Embodiment 174. The compound of any one of embodiments 1, 2, 3, 4, 5, 6, 7, 8, 164, 165, 172 and 173, preferably the compound of any one of embodiments 164, 165, 172 and 173, more preferably the compound of embodiment 173, wherein the N-terminal modification group A is selected from the group consisting of AGLU-Glutar, NTASEROL, DOTA-glu(AGLU)-APAc, DOTA and DOTA-Ttds, preferably is AGLU-Glutar or NTASEROL.

Embodiment 175. The compound of any one of embodiments 1, 2, 3, 4, 5, 6, 7, 8, 164, 165, 172, 173 and 174, preferably the compound of any one of embodiments 164, 165, 172, 173 and 174, more preferably the compound of embodiment 174, wherein the N-terminal modification group A is AGLU-Glutar.

Embodiment 176. The compound of any one of embodiments 1, 2, 3, 4, 5, 6, 7, 8, 164, 165, 172, 173 and 174, preferably the compound of any one of embodiments 164, 165, 172, 173 and 174, more preferably the compound of embodiment 174, wherein the N-terminal modification group A is NTASEROL.

Embodiment 177. The compound of any one of embodiments 1, 2, 3, 4, 5, 6, 7, 8, 164, 165, 172, 173 and 174, preferably the compound of any one of embodiments 164, 165, 172, 173 and 174, more preferably the compound of embodiment 174, wherein the N-terminal modification group A is DOTA-glu(AGLU)-APAc.

Embodiment 178. The compound of anyone of embodiments 1, 2, 3, 4, 5, 6, 7 and 8, wherein the C-terminal group C-term is
a) the residue of an α-amino acid
   optionally comprising a Z group and optionally comprising a linker interspersed between the residue of Xaa11 and the residue of an α-amino acid,
      wherein if the interspersed linker is absent, the carbonyl group R^{11b} of Xaa11 is covalently attached to the α-nitrogen of the residue of an α-amino acid,
      or wherein if the interspersed linker is present, the carbonyl group in R^{11b} of Xaa11 is covalently attached to a nitrogen atom of the linker, a carbonyl group of the linker is covalently attached to the α-nitrogen of the residue of an α-amino acid,
      and wherein a blocking group AblC is covalently attached to the carbonyl group of the residue of an α-amino acid, wherein the blocking group AblC is selected from the group consisting of NH₂ and OH, wherein the blocking group AblC preferably is NH₂, or,
b) the C-terminal dipeptide group,
   wherein the residue of the first amino acid residue is the residue of an α-amino acid and the residue of the second amino acid is the residue of an α-amino acid,
   wherein the residue of the first amino acid, the residue of the second amino acid or both the residue of the first and of the second amino acid residue optionally comprise a Z group,
   and wherein a linker is optionally interspersed between the residue of Xaa11 and of the residue of first amino acid,
   wherein if the interspersed linker is absent, the carbonyl group R^{11b} of residue Xaa11 is covalently attached to the α-nitrogen of the residue of the first amino acid and the carbonyl group of the residue of the first amino acid is covalently attached to the α-nitrogen of the residue of the second amino acid,
   or wherein if the interspersed linker is present, the carbonyl group R^{11b} of residue Xaa11 is covalently attached to a nitrogen atom of the linker, a carbonyl group of the linker is covalently attached to the α-nitrogen of the residue of the first amino acid and a carbonyl group of the residue of the first amino acid is covalently attached to the α-nitrogen of the residue of the second amino acid,
   and wherein a blocking group Abl is covalently attached to the carbonyl group of the residue of second amino acid, wherein the blocking group AblC is selected from the group consisting of NH₂ and OH, wherein the blocking group AblC preferably is NH₂, or
c) a Z group.

Embodiment 179. The compound of any one of embodiments 1, 2, 3, 4, 5, 6, 7, 8, 162, 166, 167, 168 and 178, preferably the compound of any one of embodiments 166, 167, 168 and 178, wherein under the proviso that Xaa11 is Cys, the C-terminal group C-term is
a) selected from the group consisting APAc-glu(AGLU)-NH₂, APAc-Lys(DOTA)-NH₂, Ttds-Lys(DOTA)-NH₂, glu(AGLU)-NH₂, Lys(DOTA)-NH₂, lys(DOTA)-NH₂, APAc-dap(NTASEROL)-NH₂ and lys(MeO36PEG)-NH₂, or
b) APAc-glu(AGLU)-Lys(DOTA)-NH₂ or APAc-lys(MeO2kDaPEGAc)-Lys(DOTA)-NH₂, or
c) selected from the group consisting of APAc-GLUSEROL, Ape-DOTA and PP-DOTA.

Embodiment 180. The compound of any one of embodiments 1, 2, 3, 4, 5, 6, 7, 8, 162, 166, 167, 168, 178 and 179, preferably the compound of any one of embodiments 166, 167, 168, 178 and 179, more preferably the compound of embodiment 179, wherein under the proviso that Xaa11 is Cys, the C-terminal group C-term is
a) selected from the group consisting APAc-glu(AGLU)-NH₂, APAc-Lys(DOTA)-NH₂ and APAc-dap(NTASEROL)-NH₂, or
b) APAc-glu(AGLU)-Lys(DOTA)-NH₂, or
c) APAc-GLUSEROL.

Embodiment 181. The compound of any one of embodiments 1, 2, 3, 4, 5, 6, 7, 8, 163, 169, 170, 171 and 178, preferably the compound of any one of embodiments 169, 170, 171 and 178, wherein the C-terminal group C-term is
a) selected from the group consisting APAc-glu(AGLU)-NH₂, APAc-Lys(DOTA)-NH₂, Ttds-Lys(DOTA)-NH₂, glu(AGLU)-NH₂, Lys(DOTA)-NH₂, lys(DOTA)-NH₂, APAc-dap(NTASEROL)-NH₂ and lys(MeO36PEG)-NH₂, or
b) APAc-glu(AGLU)-Lys(DOTA)-NH₂ or APAc-lys(MeO2kDaPEGAc)-Lys(DOTA)-NH₂, or
c) selected from the group consisting of APAc-GLUSEROL, Ape-DOTA and PP-DOTA.

Embodiment 182. The compound of any one of embodiments 1, 2, 3, 4, 5, 6, 7, 8, 163, 169, 170, 171, 178 and 181, preferably the compound of any one of embodiments 169, 170, 171, 178 and 181, more preferably the compound of embodiment 181, wherein under the proviso that Xaa11 is Cys, the C-terminal group C-term is
a) selected from the group consisting APAc-glu(AGLU)-NH₂, APAc-Lys(DOTA)-NH₂ and APAc-dap(NTASEROL)-NH₂, or
b) APAc-glu(AGLU)-Lys(DOTA)-NH₂, or
c) APAc-GLUSEROL.

Embodiment 183. The compound of any one of embodiments 1, 2, 3, 4, 5, 6, 7, 8, 164, 172, 173, 174, 175, 176, 177 and 178, preferably the compound of any one of embodiments 172, 173, 174, 175, 176, 177 and 178, wherein the C-terminal group C-term is
a) selected from the group consisting APAc-glu(AGLU)-NH₂, APAc-Lys(DOTA)-NH₂, Ttds-Lys(DOTA)-NH₂, glu(AGLU)-NH₂, Lys(DOTA)-NH₂, lys(DOTA)-NH₂, APAc-dap(NTASEROL)-NH₂ and lys(MeO36PEG)-NH₂, or
b) APAc-glu(AGLU)-Lys(DOTA)-NH₂ or APAc-lys(MeO2kDaPEGAc)-Lys(DOTA)-NH₂, or
c) selected from the group consisting of APAc-GLUSEROL, Ape-DOTA and PP-DOTA.

Embodiment 184. The compound of any one of embodiments 1, 2, 3, 4, 5, 6, 7, 8, 164, 172, 173, 174, 175, 176, 177, 178 and 183, preferably the compound of any one of embodiments 172, 173, 174, 175, 176, 177, 178 and 183, more preferably the compound of embodiment 183, wherein under the proviso that Xaa11 is Cys, the C-terminal group C-term is
a) selected from the group consisting APAc-glu(AGLU)-NH₂, APAc-Lys(DOTA)-NH₂ and APAc-dap(NTASEROL)-NH₂, or
b) APAc-glu(AGLU)-Lys(DOTA)-NH₂, or
c) APAc-GLUSEROL.

Embodiment 185. The compound of any one of embodiments 1, 2, 3, 4, 5, 6, 7, 8, 164, 172, 173, 174, 175, 176, 177, 178, 183 and 184, preferably the compound of any one of embodiments 172, 173, 174, 175, 176, 177, 178, 183 and 184, more preferably the compound of embodiment 184, wherein under the proviso that Xaa11 is Cys, the C-terminal group C-term is APAc-glu(AGLU)-NH₂.

Embodiment 186. The compound of any one of embodiments 1, 2, 3, 4, 5, 6, 7, 8, 164, 172, 173, 174, 175, 176, 177, 178, 183 and 184, preferably the compound of any one of embodiments 172, 173, 174, 175, 176, 177, 178, 183 and 184, more preferably the compound of embodiment 184, wherein under the proviso that Xaa11 is Cys, the C-terminal group C-term is APAc-glu(AGLU)-Lys(DOTA)-NH₂.

Embodiment 187. The compound of any one of embodiments 1, 2, 3, 4, 5, 6, 7, 8, 164, 172, 173, 174, 175, 176, 177, 178, 183 and 184, preferably the compound of any one of embodiments 172, 173, 174, 175, 176, 177, 178, 183 and 184, more preferably the compound of embodiment 184, wherein under the proviso that Xaa11 is Cys, the C-terminal group C-term is APAc-GLUSEROL.

Embodiment 188. The compound of any one of embodiments 1, 2, 3, 4, 5, 6, 7, 8, 162, 163, 164, 165, 166, 167, 168, 169, 170, 171, 172, 173, 174, 175, 176, 177, 178, 179, 180, 181, 182, 183, 184, 185, 186 and 187, preferably the compound of any one of embodiments 162, 163, 164, 165, 166, 167, 168, 169, 170, 171, 172, 173, 174, 175, 176, 177, 178, 179, 180, 181, 182, 183, 184, 185, 186 and 187, wherein the compound comprises two or more Z groups, preferably two or three Z groups, more preferably two Z groups.

Embodiment 189. The compound of any one of embodiments 1, 2, 3, 4, 5, 6, 7, 8, 162, 163, 164, 165, 166, 167, 168, 169, 170, 171, 172, 173, 174, 175, 176, 177, 178, 179, 180, 181, 182, 183, 184, 185, 186, 187 and 188, preferably the compound of any one of embodiments 162, 163, 164, 165, 166, 167, 168, 169, 170, 171, 172, 173, 174, 175, 176, 177, 178, 179, 180, 181, 182, 183, 184, 185, 186, 187 and 188, more preferably the compound of embodiment 188, wherein one of the two or more Z groups is a chelator optionally comprising a linker and wherein the chelator optionally comprising a linker
a) is covalently attached to Xaa8, wherein Xaa8 is Apc or is Amk,
b) is part of or forms the N-terminal modification group A, wherein the N-terminal modification group A is selected from the group comprising DOTA, DOTA-Ttds, DOTA-APAc,
c) is covalently attached to the residue of an α-amino acids of the C-terminal group C-term, wherein the C-terminal group C-term is selected from the group comprising APAc-Lys(DOTA)-NH₂, Ttds-Lys(DOTA)-NH₂, Lys(DOTA)-NH₂ and lys(DOTA)-NH₂
or
d) is covalently attached to one of the two residues of the amino acids of the C-terminal dipeptide group, wherein the C-terminal group C-term is APAc-glu(AGLU)-Lys(DOTA)-NH₂ or APAc-lys(MeO2kDaPEGAc)-Lys(DOTA)-NH₂.

Embodiment 190. The compound of any one of embodiments 1, 2, 3, 4, 5, 6, 7, 8, 162, 163, 164, 165, 166, 167, 168, 169, 170, 171, 172, 173, 174, 175, 176, 177, 178, 179, 180, 181, 182, 183, 184, 185, 186, 187 and 188, preferably the compound of any one of embodiments 162, 163, 164, 165, 166, 167, 168, 169, 170, 171, 172, 173, 174, 175, 176, 177, 178, 179, 180, 181, 182, 183, 184, 185, 186, 187 and 188, more preferably the compound of embodiment 188, wherein one of the two or more Z groups is a biodistribution modifier optionally comprising a linker, wherein the biodistribution modifier optionally comprising a linker
a) is part of or forms the N-terminal modification group A, wherein the N-terminal modification group A is selected from the group comprising of AGLU-Glutar, NTASEROL, AGLU-Glutar-APAc, MeO36PEG-APAc and MeO2kDaPEGAc, preferably is AGLU-Glutar or NTASEROL,
b) is covalently attached to the residue of an α-amino acids of the C-terminal group C-term, wherein C-term is selected from the group consisting APAc-glu(AGLU)-NH₂, glu(AGLU)-NH₂, Lys(DOTA)-NH₂, lys(DOTA)-NH₂, APAc-dap(NTASEROL)-NH₂ and lys(MeO36PEG)-NH₂,
c) is covalently attached to one of the two residues of an amino acid of the C-terminal dipeptide group, wherein the C-terminal group C-term is APAc-glu(AGLU)-Lys(DOTA)-NH₂ or APAc-lys(MeO2kDaPEGAc)-Lys(DOTA)-NH₂,
or
d) is part of or forms the C-terminal group C-term, wherein the C-terminal group C-term is APAc-GLUSEROL.

Embodiment 191. The compound of any one of embodiments 1, 2, 3, 4, 5, 6, 7, 8, 162, 163, 164, 165, 166, 167, 168, 169, 170, 171, 172, 173, 174, 175, 176, 177, 178, 179, 180, 181, 182, 183, 184, 185, 186, 187 and 188, preferably the compound of any one of embodiments 162, 163, 164, 165, 166, 167, 168, 169, 170, 171, 172, 173, 174, 175, 176, 177, 178, 179, 180, 181, 182, 183, 184, 185, 186, 187 and 188, more preferably the compound of embodiment 188, wherein one of the two or more Z groups is a combination of a chelator optionally comprising a linker, a bio-distribution modifier optionally comprising a linker, and an amino acid residue linking the chelator optionally comprising a linker and the bio-distribution modifier optionally comprising a linker, wherein the combination of a chelator and a bio-distribution modifier
is part of or forms the N-terminal modification group A, wherein the N-terminal modification group A is selected from the group comprising of DOTA-glu(AGLU), DOTA-glu(AGLU)-APAc, DOTA-O2Oc-glu(AGLU), DOTA-glu(AGLU)-O2Oc, DOTA-PEG6-glu(AGLU), and DOTA-glu(AGLU)-PEG6.

Embodiment 192. The compound of any one of embodiments 1, 2, 3, 4, 5, 6, 7, 8, 162, 163, 164, 165, 166, 167, 168, 169, 170, 171, 172, 173, 174, 175, 176, 177, 178, 179, 180, 181, 182, 183, 184, 185, 186, 187, 188, 189, 190 and 191, preferably the compound of any one of embodiments 162, 163, 164, 165, 166, 167, 168, 169, 170, 171, 172, 173, 174, 175, 176, 177, 178, 179, 180, 181, 182, 183, 184, 185, 186, 187, 188, 189, 190 and 191, more preferably the compound of any one of embodiments 188, 189, 190 and 191, wherein the compound comprises three Z groups,
wherein a first of the three Z groups is a chelator,
   wherein the chelator is covalently attached to Xaa8,
   wherein Xaa8 is a Apc residue and wherein the chelator is DOTA,
and
wherein a second of the three Z groups is bio-distribution modifier optionally comprising a linker,
   wherein the bio-distribution modifier and the linker form the N-terminal modification group A,
   wherein the N-terminal modification group A is AGLU-Glutar,
and
wherein a third of the three Z groups is a bio-distribution modifier,
   wherein the bio-distribution modifier is covalently attached to the residue of an α-amino acid of the C-terminal group C-term
   and wherein the C-terminal group C-term is APAc-glu(AGLU)-NH₂.

Embodiment 193. The compound of any one of embodiments 1, 2, 3, 4, 5, 6, 7, 8, 162, 163, 164, 165, 166, 167, 168, 169, 170, 171, 172, 173, 174, 175, 176, 177, 178, 179, 180, 181, 182, 183, 184, 185, 186, 187, 188, 189, 190 and 191, preferably the compound of any one of embodiments 162, 163, 164, 165, 166, 167, 168, 169, 170, 171, 172, 173, 174, 175, 176, 177, 178, 179, 180, 181, 182, 183, 184, 185, 186, 187, 188, 189, 190 and 191, more preferably the compound of any one of embodiments 188, 189, 190 and 191, wherein the compound comprises two Z groups,
wherein one Z group is a combination of a chelator optionally comprising a linker, a bio-distribution modifier optionally comprising a linker, and an amino acid residue linking the chelator optionally comprising a linker and the bio-distribution modifier optionally comprising a linker,
   wherein the combination of a chelator and a bio-distribution modifier is part of or forms the N-terminal modification group A,
   wherein the N-terminal modification group A is DOTA-glu(AGLU)
and
wherein the other Z group is a bio-distribution modifier,
   wherein the bio-distribution modifier is covalently attached to the residue of an α-amino acid of the C-terminal group C-term
   and wherein the C-terminal group C-term is APAc-glu(AGLU)-NH₂.

Embodiment 194. The compound of any one of embodiments 1, 2, 3, 4, 5, 6, 7, 8, 162, 163, 164, 165, 166, 167, 168, 169, 170, 171, 172, 173, 174, 175, 176, 177, 178, 179, 180, 181, 182, 183, 184, 185, 186, 187, 188, 189, 190 and 191, preferably the compound of any one of embodiments 162, 163, 164, 165, 166, 167, 168, 169, 170, 171, 172, 173, 174, 175, 176, 177, 178, 179, 180, 181, 182, 183, 184, 185, 186, 187, 188, 189, 190 and 191, more preferably the compound of any one of embodiments 188, 189, 190 and 191, wherein the compound comprises three Z groups,
wherein a first of the three Z groups is a chelator,
   wherein the chelator is covalently attached to one of the two residues of the amino acids of the C-terminal dipeptide group,
wherein a second of the three Z groups is a bio-distribution modifier,
   wherein the bio-distribution modifier is covalently attached to the other of the two residues of the amino acids of the C-terminal dipeptide group,
   wherein the C-terminal dipeptide group is the C-terminal group C-term and wherein C-term is APAc-glu(AGLU)-Lys(DOTA)-NH₂,
and
   wherein a third of the three Z groups is a bio-distribution modifier optionally comprising a linker, wherein bio-distribution modifier and the linker form the N-terminal modification group A and wherein the N-terminal modification group A is AGLU-Glutar.

Embodiment 195. The compound of any one of embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 138, 139, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193 and 194, wherein the compound is selected from the group consisting of compound AGLU-Glutar-Asp-Trp-5Clw-Tap-Glu-leu-[Cys(2Lut)-Apc(DOTA)-Leu-Ile-Cys]-APAc-glu(AGLU)-NH₂ (GIP-001, SEQ ID NO: 5) of the following formula

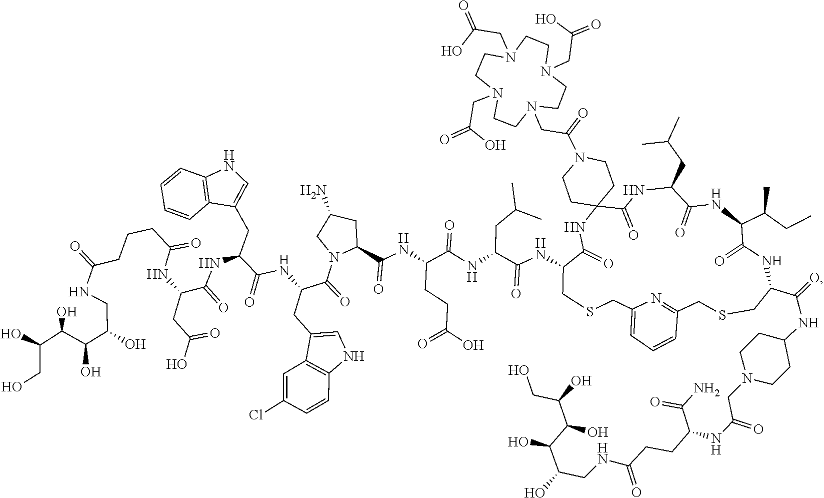 [SMILES: CC[C@H](C)[C@@H]1NC(=O)[C@H](CC(C)C)NC(=O)C2(CCN(C(=O)CN3CCN(CC(=O)O)CCN(CC(=O)O)CCN(CC(=O)O)CC3)CC2)NC(=O)[C@@H](NC(=O)[C@@H](CC(C)C)NC(=O)[C@H](CCC(=O)O)NC(=O)[C@@H]2C[C@@H](N)CN2C(=O)[C@H](Cc2c[nH]c3ccc(Cl)cc23)NC(=O)[C@H](Cc2c[nH]c3ccccc23)NC(=O)[C@H](CC(=O)O)NC(=O)CCCC(=O)NC[C@H](O)[C@@H](O)[C@H](O)[C@H](O)CO)CSCc2cccc(n2)CSC[C@@H](C(=O)NC2CCN(CC(=O)N[C@H](CCC(=O)NC[C@H](O)[C@@H](O)[C@H](O)[C@H](O)CO)C(N)=O)CC2)NC1=O]

compound AGLU-Glutar-Asp-Trp-5Clw-Hyp-Glu-leu-[Cys(2Lut)-Apc(DOTA)-Leu-Ile-Cys]-APAc-glu(AGLU)-NH₂ (GIP-002, SEQ ID NO: 6) of the following formula

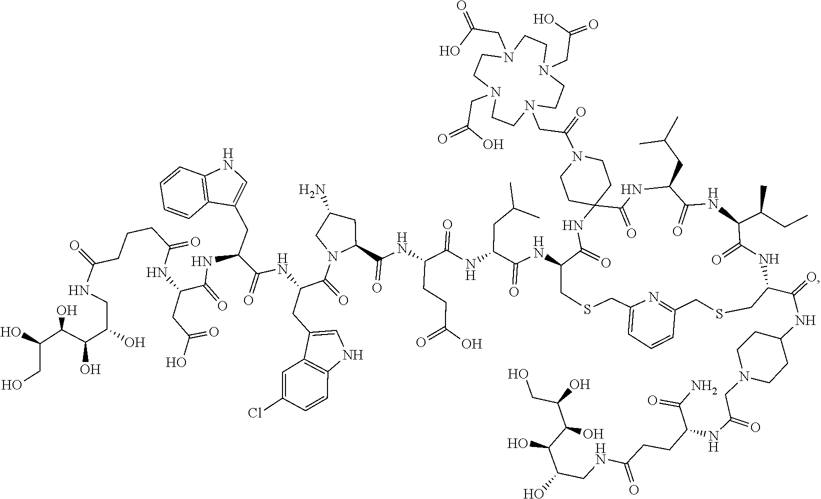 [SMILES: CC[C@H](C)[C@@H]1NC(=O)[C@H](CC(C)C)NC(=O)C2(CCN(C(=O)CN3CCN(CC(=O)O)CCN(CC(=O)O)CCN(CC(=O)O)CC3)CC2)NC(=O)[C@H](NC(=O)[C@@H](CC(C)C)NC(=O)[C@H](CCC(=O)O)NC(=O)[C@@H]2C[C@@H](N)CN2C(=O)[C@H](Cc2c[nH]c3ccc(Cl)cc23)NC(=O)[C@H](Cc2c[nH]c3ccccc23)NC(=O)[C@H](CC(=O)O)NC(=O)CCCC(=O)NC[C@H](O)[C@@H](O)[C@H](O)[C@H](O)CO)CSCc2cccc(n2)CSC[C@@H](C(=O)NC2CCN(CC(=O)N[C@H](CCC(=O)NC[C@H](O)[C@@H](O)[C@H](O)[C@H](O)CO)C(N)=O)CC2)NC1=O]

compound DOTA-glu(AGLU)-APAc-Asp-Trp-5Clw-Hyp-Glu-leu-[Cys(2Lut)-Apc(AGLU-Glutar)-Leu-Ile-Cys]-APAc-glu(AGLU)-NH₂ (GIP-003, SEQ ID NO: 7) of the following formula

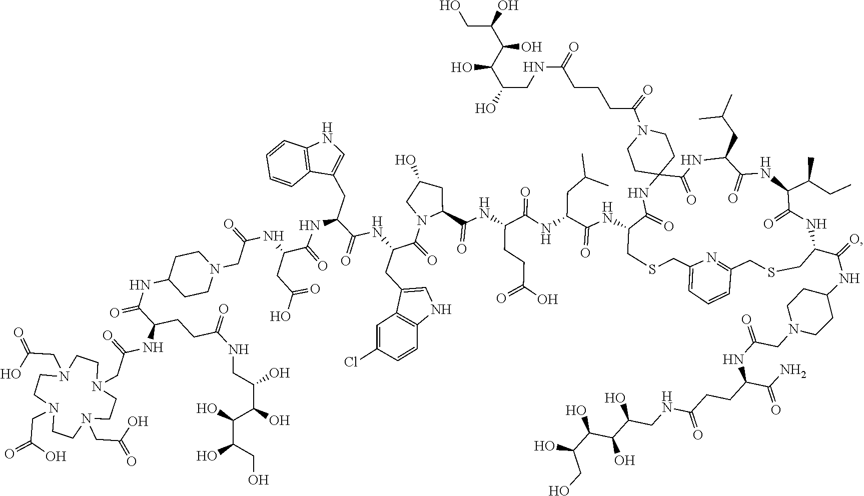 [SMILES: CC[C@H](C)[C@@H]1NC(=O)[C@H](CC(C)C)NC(=O)C2(CCN(C(=O)CCCC(=O)NC[C@H](O)[C@@H](O)[C@H](O)[C@H](O)CO)CC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CC(=O)O)NC(=O)CN2CCC(NC(=O)[C@@H](CCC(=O)NC[C@H](O)[C@@H](O)[C@H](O)[C@H](O)CO)NC(=O)CN3CCN(CC(=O)O)CCN(CC(=O)O)CCN(CC(=O)O)CC3)CC2)CSCc2cccc(n2)CSC[C@@H](C(=O)NC2CCN(CC(=O)N[C@H](CCC(=O)NC[C@H](O)[C@@H](O)[C@H](O)[C@H](O)CO)C(N)=O)CC2)NC1=O]

compound AGLU-Glutar-Asp-Trp-5Clw-Hyp-Glu-leu-[Cys(2Lut)-Thp-Leu-Ile-Cys]-APAc-glu(AGLU)-Lys(DOTA)-NH₂ (GIP-004, SEQ ID NO: 8) of the following formula

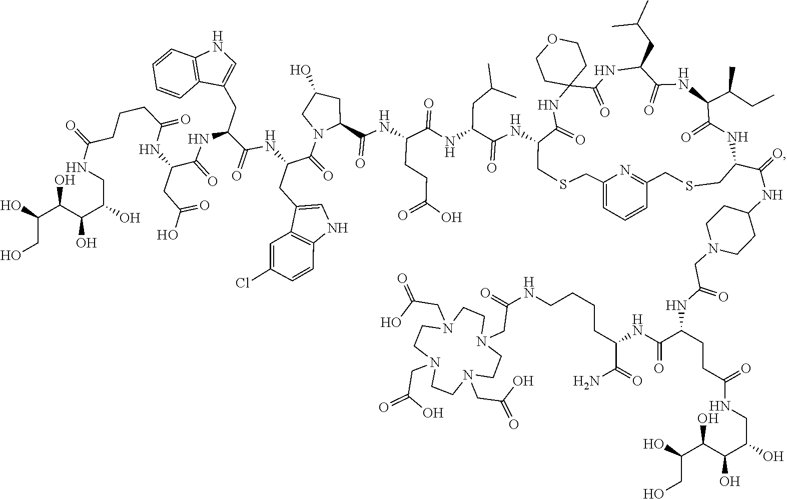 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CC(=O)O)NC(=O)CCCC(=O)NC[C@H](O)[C@@H](O)[C@H](O)[C@H](O)CO)CSCc2cccc(n2)CSC[C@@H](C(=O)NC2CCN(CC(=O)N[C@H](CCC(=O)NC[C@H](O)[C@@H](O)[C@H](O)[C@H](O)CO)C(=O)N[C@@H](CCCCNC(=O)CN3CCN(CC(=O)O)CCN(CC(=O)O)CCN(CC(=O)O)CC3)C(N)=O)CC2)NC1=O]

compound DOTA-glu(AGLU)-APAc-Asp-Trp-5Clw-Hyp-Glu-leu-[Cys(2Lut)-Thp-Leu-Ile-Cys]-APAc-glu(AGLU)-NH₂ (GIP-005, SEQ ID NO: 9) of the following formula

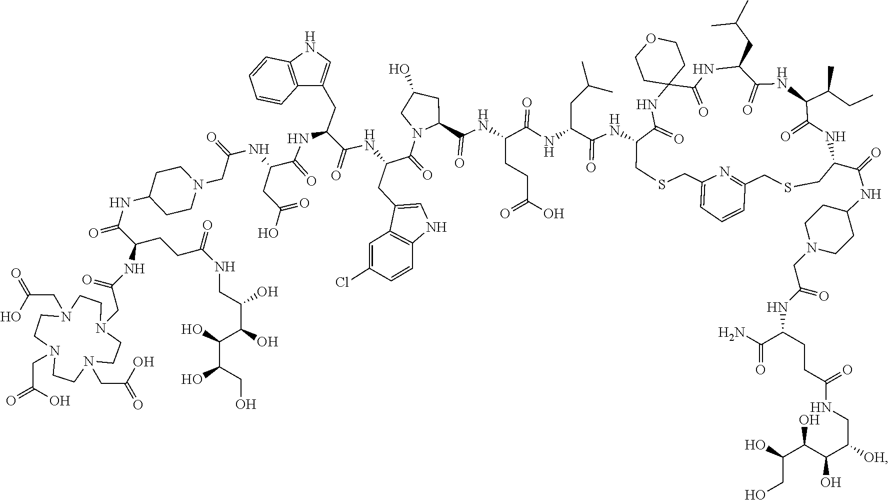 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CC(=O)O)NC(=O)CN2CCC(NC(=O)[C@@H](CCC(=O)NC[C@H](O)[C@@H](O)[C@H](O)[C@H](O)CO)NC(=O)CN3CCN(CC(=O)O)CCN(CC(=O)O)CCN(CC(=O)O)CC3)CC2)CSCc2cccc(n2)CSC[C@@H](C(=O)NC2CCN(CC(=O)N[C@H](CCC(=O)NC[C@H](O)[C@@H](O)[C@H](O)[C@H](O)CO)C(N)=O)CC2)NC1=O]

compound AGLU-Glutar-APAc-Asp-Trp-5Clw-Tap(AGLU-Glutar)-Glu-leu-[Cys(2Lut)-Apc(DOTA)-Leu-Ile-Cys]-APAc-glu(AGLU)-NH₂ (GIP-006, SEQ ID NO: 10) of the following formula

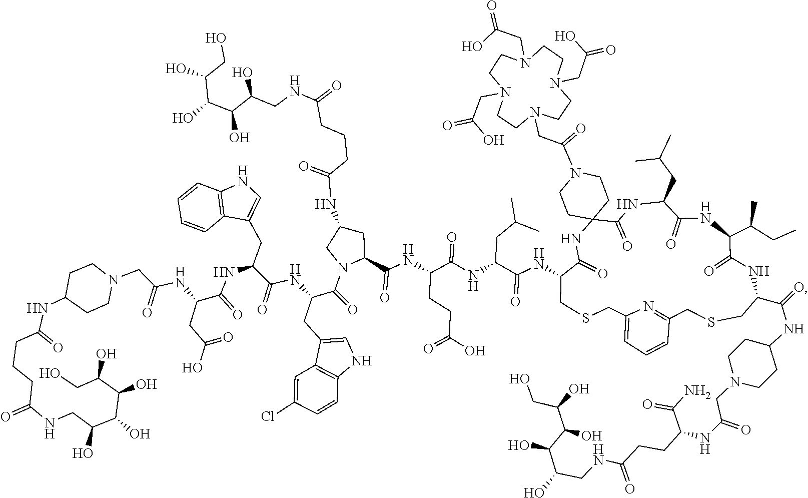 [SMILES: CC[C@H](C)[C@@H]1NC(=O)[C@H](CC(C)C)NC(=O)C2(CCN(C(=O)CN3CCN(CC(=O)O)CCN(CC(=O)O)CCN(CC(=O)O)CC3)CC2)NC(=O)[C@@H](NC(=O)[C@@H](CC(C)C)NC(=O)[C@H](CCC(=O)O)NC(=O)[C@@H]2C[C@@H](NC(=O)CCCC(=O)NC[C@H](O)[C@@H](O)[C@H](O)[C@H](O)CO)CN2C(=O)[C@H](Cc2c[nH]c3ccc(Cl)cc23)NC(=O)[C@H](Cc2c[nH]c3ccccc23)NC(=O)[C@H](CC(=O)O)NC(=O)CN2CCC(NC(=O)CCCC(=O)NC[C@H](O)[C@@H](O)[C@H](O)[C@H](O)CO)CC2)CSCc2cccc(n2)CSC[C@@H](C(=O)NC2CCN(CC(=O)N[C@H](CCC(=O)NC[C@H](O)[C@@H](O)[C@H](O)[C@H](O)CO)C(N)=O)CC2)NC1=O]

compound AGLU-Glutar-APAc-Asp-Trp-5Clw-Tap(AGLU-Glutar)-Glu-leu-[Cys(2Lut)-Apc(DOTA-glu(AGLU))-Leu-Ile-Cys]-APAc-glu(AGLU)-NH₂ (GIP-007, SEQ ID NO: 11) of the following formula

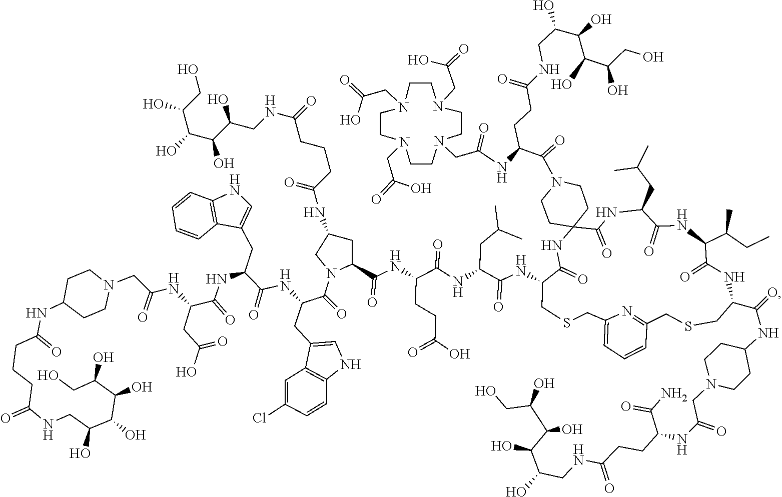

compound AGLU-Glutar-APAc-Asp-Trp-5Clw-Tap-Glu-leu-[Cys(2Lut)-Apc(DOTA-glu(AGLU))-Leu-Ile-Cys]-APAc-glu(AGLU)-NH₂ (GIP-008, SEQ ID NO: 12) of the following formula

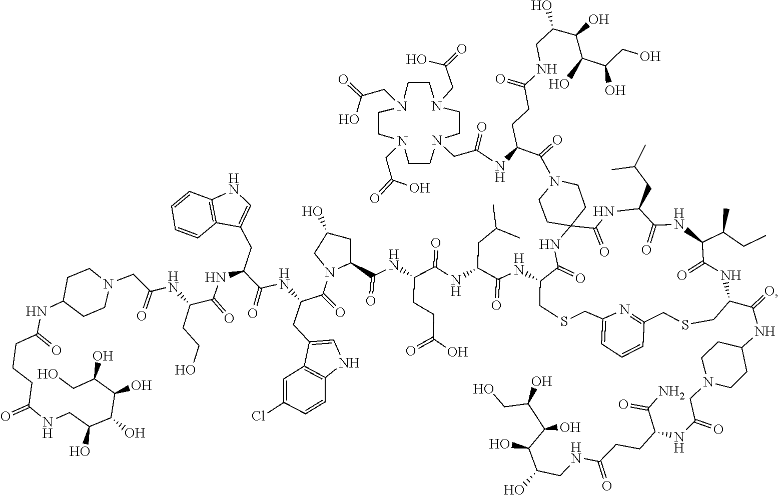 [SMILES: CC[C@H](C)[C@@H]1NC(=O)[C@H](CC(C)C)NC(=O)C2(CCN(C(=O)[C@H](CCC(=O)NC[C@H](O)[C@@H](O)[C@H](O)[C@H](O)CO)NC(=O)CN3CCN(CC(=O)O)CCN(CC(=O)O)CCN(CC(=O)O)CC3)CC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CCO)NC(=O)CN2CCC(NC(=O)CCCC(=O)NC[C@H](O)[C@@H](O)[C@H](O)[C@H](O)CO)CC2)CSCc2cccc(n2)CSC[C@@H](C(=O)NC2CCN(CC(=O)N[C@H](CCC(=O)NC[C@H](O)[C@@H](O)[C@H](O)[C@H](O)CO)C(N)=O)CC2)NC1=O]

compound DOTA-glu(AGLU)-O2Oc-Asp-Trp-5Clw-Tap-Glu-leu-[Cys(2Lut)-Apc(AGLU-Glutar)-Leu-Ile-Cys]-APAc-glu(AGLU)-NH₂ (GIP-009, SEQ ID NO: 13) of the following formula

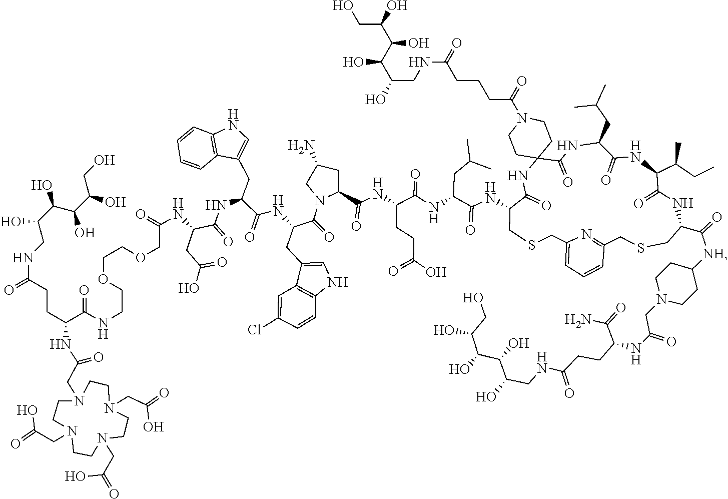 [SMILES: CC[C@H](C)[C@@H]1NC(=O)[C@H](CC(C)C)NC(=O)C2(CCN(C(=O)CCCC(=O)NC[C@H](O)[C@@H](O)[C@H](O)[C@H](O)CO)CC2)NC(=O)[C@@H](NC(=O)[C@@H](CC(C)C)NC(=O)[C@H](CCC(=O)O)NC(=O)[C@@H]2C[C@@H](N)CN2C(=O)[C@H](Cc2c[nH]c3ccc(Cl)cc23)NC(=O)[C@H](Cc2c[nH]c3ccccc23)NC(=O)[C@H](CC(=O)O)NC(=O)COCCOCCNC(=O)[C@@H](CCC(=O)NC[C@H](O)[C@@H](O)[C@H](O)[C@H](O)CO)NC(=O)CN2CCN(CC(=O)O)CCN(CC(=O)O)CCN(CC(=O)O)CC2)CSCc2cccc(n2)CSC[C@@H](C(=O)NC2CCN(CC(=O)N[C@H](CCC(=O)NC[C@H](O)[C@@H](O)[C@H](O)[C@H](O)CO)C(N)=O)CC2)NC1=O]

compound AGLU-Glutar-Asp-Trp-5Clw-Hyp-Glu-leu-[Cys(2Lut)-Apc(AGLU-Glutar)-Leu-Ile-Cys]-APAc-Lys(DOTA)-NH₂ (GIP-010, SEQ ID NO: 14) of the following formula

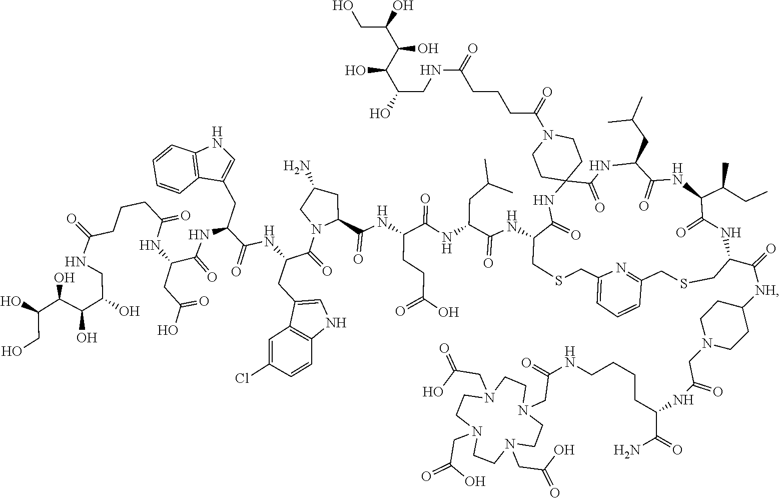 [SMILES: CC[C@H](C)[C@@H]1NC(=O)[C@H](CC(C)C)NC(=O)C2(CCN(C(=O)CCCC(=O)NC[C@H](O)[C@@H](O)[C@H](O)[C@H](O)CO)CC2)NC(=O)[C@@H](NC(=O)[C@@H](CC(C)C)NC(=O)[C@H](CCC(=O)O)NC(=O)[C@@H]2C[C@@H](N)CN2C(=O)[C@H](Cc2c[nH]c3ccc(Cl)cc23)NC(=O)[C@H](Cc2c[nH]c3ccccc23)NC(=O)[C@H](CC(=O)O)NC(=O)CCCC(=O)NC[C@H](O)[C@@H](O)[C@H](O)[C@H](O)CO)CSCc2cccc(n2)CSC[C@@H](C(=O)NC2CCN(CC(=O)N[C@@H](CCCCNC(=O)CN3CCN(CC(=O)O)CCN(CC(=O)O)CCN(CC(=O)O)CC3)C(N)=O)CC2)NC1=O]

compound AGLU-Glutar-APAc-Asp-Trp-5Clw-Hyp-Glu-leu-[Cys(2Lut)-Apc(DOTA)-Leu-Ile-Cys]-APAc-glu(AGLU)-NH₂ (GIP-011, SEQ ID NO: 15) of the following formula

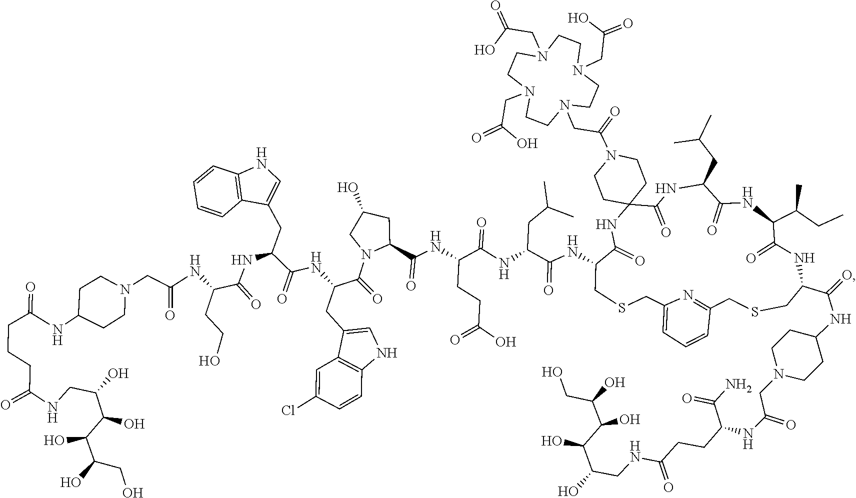 [SMILES: CC[C@H](C)[C@@H]1NC(=O)[C@H](CC(C)C)NC(=O)C2(CCN(C(=O)CN3CCN(CC(=O)O)CCN(CC(=O)O)CCN(CC(=O)O)CC3)CC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CCO)NC(=O)CN2CCC(NC(=O)CCCC(=O)NC[C@H](O)[C@@H](O)[C@H](O)[C@H](O)CO)CC2)CSCc2cccc(n2)CSC[C@@H](C(=O)NC2CCN(CC(=O)N[C@H](CCC(=O)NC[C@H](O)[C@@H](O)[C@H](O)[C@H](O)CO)C(N)=O)CC2)NC1=O]

compound DOTA-APAc-Asp-Trp-5Clw-Hyp-Glu-leu-[Cys(2Lut)-Apc(AGLU-Glutar)-Leu-Ile-Cys]-APAc-glu(AGLU)-NH₂ (GIP-012, SEQ ID NO: 16) of the following formula

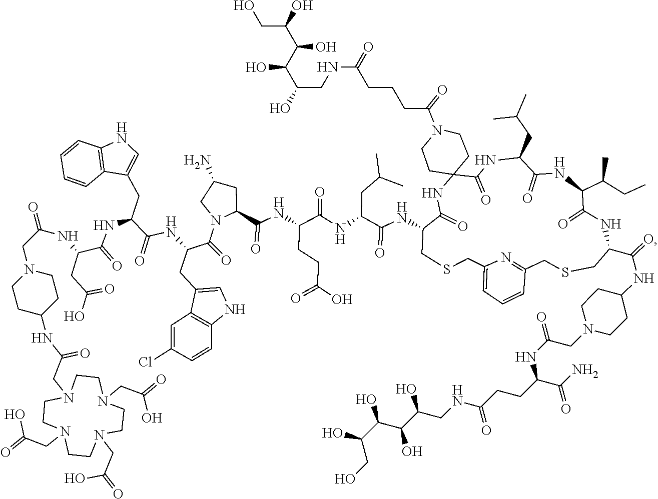 [SMILES: CC[C@H](C)[C@@H]1NC(=O)[C@H](CC(C)C)NC(=O)C2(CCN(C(=O)CCCC(=O)NC[C@H](O)[C@@H](O)[C@H](O)[C@H](O)CO)CC2)NC(=O)[C@@H](NC(=O)[C@@H](CC(C)C)NC(=O)[C@H](CCC(=O)O)NC(=O)[C@@H]2C[C@@H](N)CN2C(=O)[C@H](Cc2c[nH]c3ccc(Cl)cc23)NC(=O)[C@H](Cc2c[nH]c3ccccc23)NC(=O)[C@H](CC(=O)O)NC(=O)CN2CCC(NC(=O)CN3CCN(CC(=O)O)CCN(CC(=O)O)CCN(CC(=O)O)CC3)CC2)CSCc2cccc(n2)CSC[C@@H](C(=O)NC2CCN(CC(=O)N[C@H](CCC(=O)NC[C@H](O)[C@@H](O)[C@H](O)[C@H](O)CO)C(N)=O)CC2)NC1=O]

compound AGLU-Glutar-APAc-Asp-Trp-5Clw-Tap-Glu-leu-[Cys(2Lut)-Apc(DOTA)-Leu-Ile-Cys]-APAc-glu(AGLU)-NH₂ (GIP-013, SEQ ID NO: 17) of the following formula

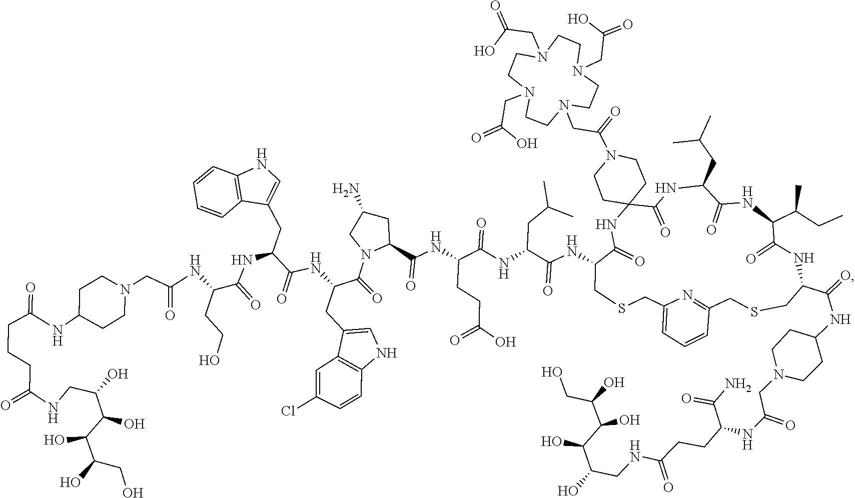 [SMILES: CC[C@H](C)[C@@H]1NC(=O)[C@H](CC(C)C)NC(=O)C2(CCN(C(=O)CN3CCN(CC(=O)O)CCN(CC(=O)O)CCN(CC(=O)O)CC3)CC2)NC(=O)[C@@H](NC(=O)[C@@H](CC(C)C)NC(=O)[C@H](CCC(=O)O)NC(=O)[C@@H]2C[C@@H](N)CN2C(=O)[C@H](Cc2c[nH]c3ccc(Cl)cc23)NC(=O)[C@H](Cc2c[nH]c3ccccc23)NC(=O)[C@H](CCO)NC(=O)CN2CCC(NC(=O)CCCC(=O)NC[C@H](O)[C@@H](O)[C@H](O)[C@H](O)CO)CC2)CSCc2cccc(n2)CSC[C@@H](C(=O)NC2CCN(CC(=O)N[C@H](CCC(=O)NC[C@H](O)[C@@H](O)[C@H](O)[C@H](O)CO)C(N)=O)CC2)NC1=O]

compound AGLU-Glutar-APAc-Asp-Trp-5Brw-Tap-Glu-leu-[Cys(2Lut)-Apc(DOTA)-Leu-Ile-Cys]-APAc-glu(AGLU)-NH₂ (GIP-014, SEQ ID NO: 18) of the following formula

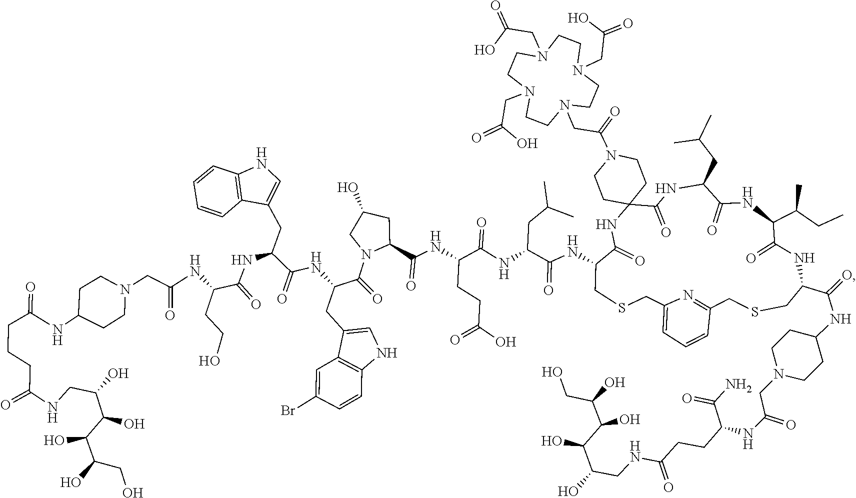 [SMILES: CC[C@H](C)[C@@H]1NC(=O)[C@H](CC(C)C)NC(=O)C2(CCN(C(=O)CN3CCN(CC(=O)O)CCN(CC(=O)O)CCN(CC(=O)O)CC3)CC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Br)cc23)NC(=O)[C@H](Cc2c[nH]c3ccccc23)NC(=O)[C@H](CCO)NC(=O)CN2CCC(NC(=O)CCCC(=O)NC[C@H](O)[C@@H](O)[C@H](O)[C@H](O)CO)CC2)CSCc2cccc(n2)CSC[C@@H](C(=O)NC2CCN(CC(=O)N[C@H](CCC(=O)NC[C@H](O)[C@@H](O)[C@H](O)[C@H](O)CO)C(N)=O)CC2)NC1=O]

compound AGLU-Glutar-Asp-Trp-5Brw-Tap-Glu-leu-[Cys(2Lut)-Apc(DOTA)-Leu-Ile-Cys]-APAc-glu(AGLU)-NH₂ (GIP-015, SEQ ID NO: 19) of the following formula

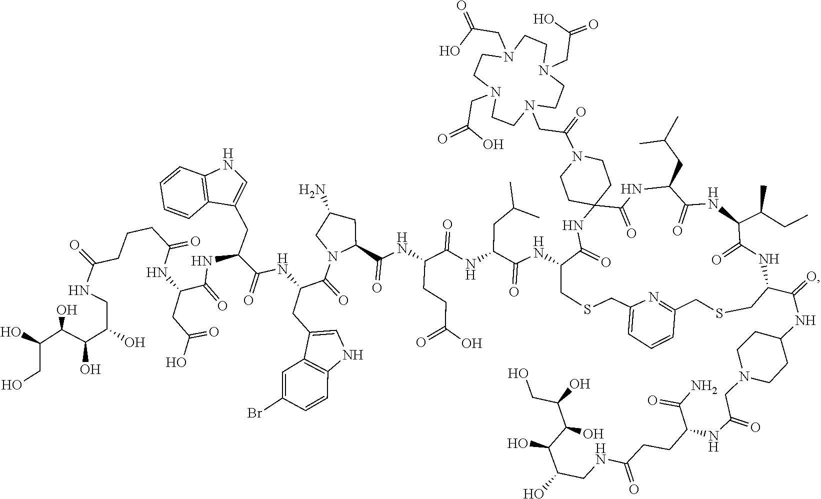 [SMILES: CC[C@H](C)[C@@H]1NC(=O)[C@H](CC(C)C)NC(=O)C2(CCN(C(=O)CN3CCN(CC(=O)O)CCN(CC(=O)O)CCN(CC(=O)O)CC3)CC2)NC(=O)[C@@H](NC(=O)[C@@H](CC(C)C)NC(=O)[C@H](CCC(=O)O)NC(=O)[C@@H]2C[C@@H](N)CN2C(=O)[C@H](Cc2c[nH]c3ccc(Br)cc23)NC(=O)[C@H](Cc2c[nH]c3ccccc23)NC(=O)[C@H](CC(=O)O)NC(=O)CCCC(=O)NC[C@H](O)[C@@H](O)[C@H](O)[C@H](O)CO)CSCc2cccc(n2)CSC[C@@H](C(=O)NC2CCN(CC(=O)N[C@H](CCC(=O)NC[C@H](O)[C@@H](O)[C@H](O)[C@H](O)CO)C(N)=O)CC2)NC1=O]

compound AGLU-Glutar-Hse-Trp-5Brw-Tap-Glu-leu-[Cys(2Lut)-Apc(DOTA)-Leu-Ile-Cys]-APAc-glu(AGLU)-NH₂ (GIP-016, SEQ ID NO: 20) of the following formula

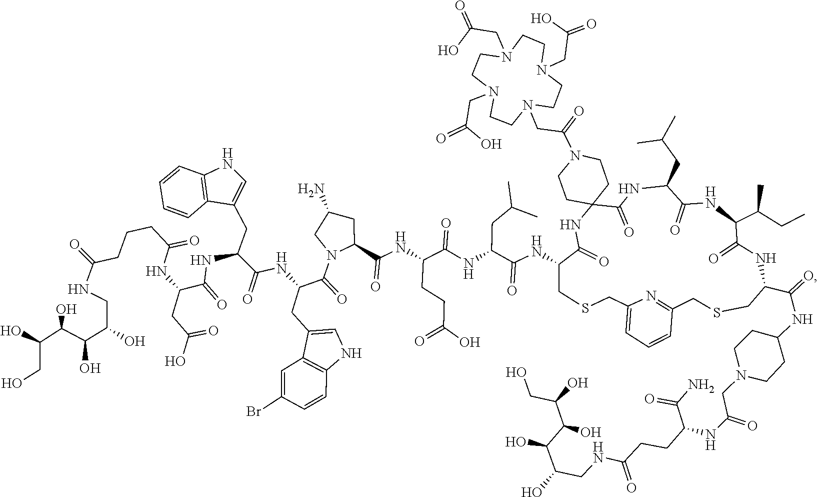 [SMILES: CC[C@H](C)[C@@H]1NC(=O)[C@H](CC(C)C)NC(=O)C2(CCN(C(=O)CN3CCN(CC(=O)O)CCN(CC(=O)O)CCN(CC(=O)O)CC3)CC2)NC(=O)[C@@H](NC(=O)[C@@H](CC(C)C)NC(=O)[C@H](CCC(=O)O)NC(=O)[C@@H]2C[C@@H](N)CN2C(=O)[C@H](Cc2c[nH]c3ccc(Br)cc23)NC(=O)[C@H](Cc2c[nH]c3ccccc23)NC(=O)[C@H](CC(=O)O)NC(=O)CCCC(=O)NC[C@H](O)[C@@H](O)[C@H](O)[C@H](O)CO)CSCc2cccc(n2)CSC[C@@H](C(=O)NC2CCN(CC(=O)N[C@H](CCC(=O)NC[C@H](O)[C@@H](O)[C@H](O)[C@H](O)CO)C(N)=O)CC2)NC1=O]

compound DOTA-glu(AGLU)-Asp-Trp-5Clw-Tap-Glu-leu-[Cys(2Lut)-Thp-Leu-Ile-Cys]-APAc-glu(AGLU)-NH₂ (GIP-017, SEQ ID NO: 21) of the following formula

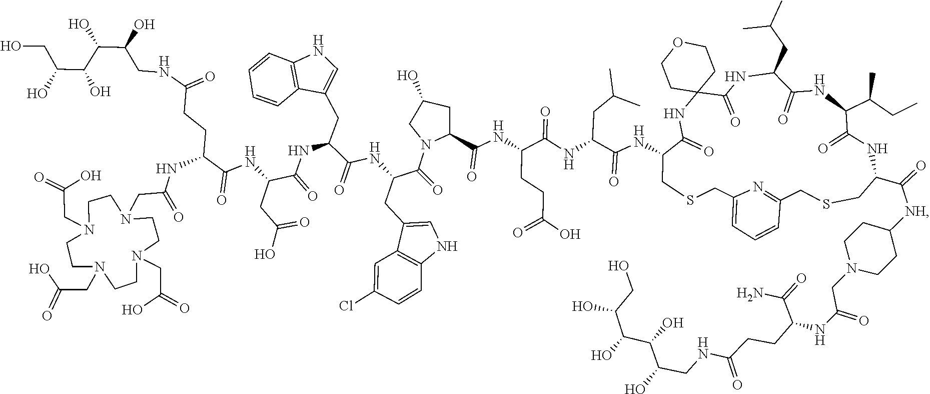 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CC(=O)O)NC(=O)[C@@H](CCC(=O)NC[C@H](O)[C@@H](O)[C@H](O)[C@H](O)CO)NC(=O)CN2CCN(CC(=O)O)CCN(CC(=O)O)CCN(CC(=O)O)CC2)CSCc2cccc(n2)CSC[C@@H](C(=O)NC2CCN(CC(=O)N[C@H](CCC(=O)NC[C@H](O)[C@@H](O)[C@H](O)[C@H](O)CO)C(N)=O)CC2)NC1=O]

compound DOTA-O2Oc-glu(AGLU)-Asp-Trp-5Clw-Tap-Glu-leu-[Cys(2Lut)-Thp-Leu-Ile-Cys]-APAc-glu(AGLU)-NH₂ (GIP-018, SEQ ID NO: 22) of the following formula

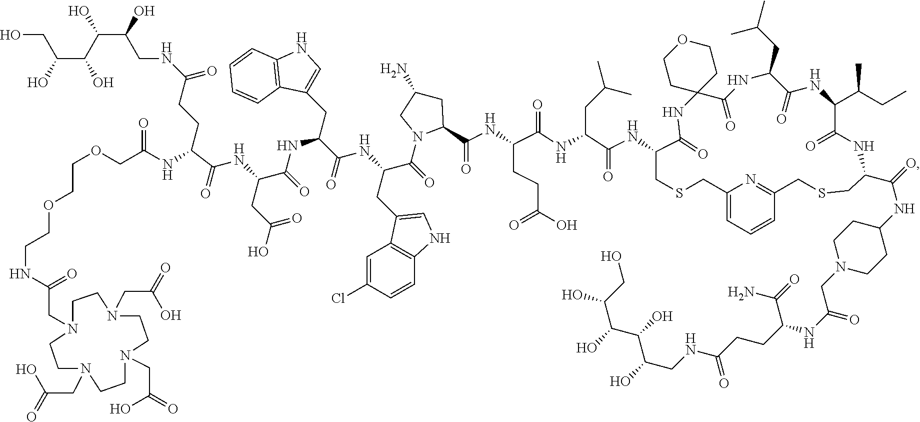 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](N)CN2C(=O)[C@H](Cc2c[nH]c3ccc(Cl)cc23)NC(=O)[C@H](Cc2c[nH]c3ccccc23)NC(=O)[C@H](CC(=O)O)NC(=O)[C@@H](CCC(=O)NC[C@H](O)[C@@H](O)[C@H](O)[C@H](O)CO)NC(=O)COCCOCCNC(=O)CN2CCN(CC(=O)O)CCN(CC(=O)O)CCN(CC(=O)O)CC2)CSCc2cccc(n2)CSC[C@@H](C(=O)NC2CCN(CC(=O)N[C@H](CCC(=O)NC[C@H](O)[C@@H](O)[C@H](O)[C@H](O)CO)C(N)=O)CC2)NC1=O]

compound DOTA-glu(AGLU)-PEG6-Asp-Trp-5Clw-Tap-Glu-leu-[Cys(2Lut)-Thp-Leu-Ile-Cys]-APAc-glu(AGLU)-NH₂ (GIP-019, SEQ ID NO: 23) of the following formula

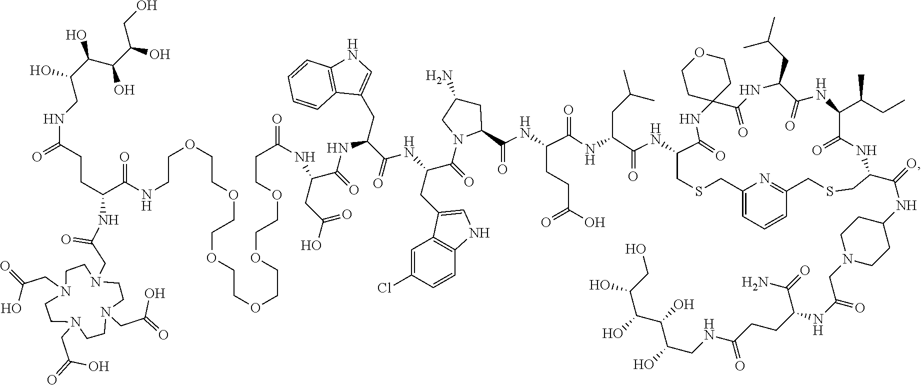 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](N)CN2C(=O)[C@H](Cc2c[nH]c3ccc(Cl)cc23)NC(=O)[C@H](Cc2c[nH]c3ccccc23)NC(=O)[C@H](CC(=O)O)NC(=O)CCOCCOCCOCCOCCOCCOCCNC(=O)[C@@H](CCC(=O)NC[C@H](O)[C@@H](O)[C@H](O)[C@H](O)CO)NC(=O)CN2CCN(CC(=O)O)CCN(CC(=O)O)CCN(CC(=O)O)CC2)CSCc2cccc(n2)CSC[C@@H](C(=O)NC2CCN(CC(=O)N[C@H](CCC(=O)NC[C@H](O)[C@@H](O)[C@H](O)[C@H](O)CO)C(N)=O)CC2)NC1=O]

compound DOTA-glu(AGLU)-O2Oc-Asp-Trp-5Clw-Tap-Glu-leu-[Cys(2Lut)-Thp-Leu-Ile-Cys]-APAc-glu(AGLU)-NH₂ (GIP-020, SEQ ID NO: 24) of the following formula

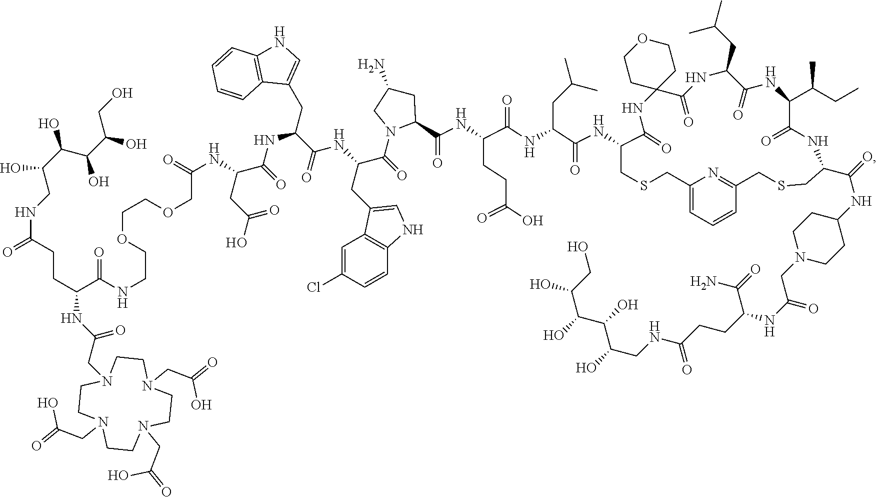 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](N)CN2C(=O)[C@H](Cc2c[nH]c3ccc(Cl)cc23)NC(=O)[C@H](Cc2c[nH]c3ccccc23)NC(=O)[C@H](CC(=O)O)NC(=O)COCCOCCNC(=O)[C@@H](CCC(=O)NC[C@H](O)[C@@H](O)[C@H](O)[C@H](O)CO)NC(=O)CN2CCN(CC(=O)O)CCN(CC(=O)O)CCN(CC(=O)O)CC2)CSCc2cccc(n2)CSC[C@@H](C(=O)NC2CCN(CC(=O)N[C@H](CCC(=O)NC[C@H](O)[C@@H](O)[C@H](O)[C@H](O)CO)C(N)=O)CC2)NC1=O]

compound HO-Succinyl-Trp-5Clw-Hyp-Glu-leu-[Cys(2Lut)-Thp-Leu-Ile-Cys]-APAc-glu(AGLU)-Lys(DOTA)-NH₂ (GIP-021, SEQ ID NO: 25) of the following formula

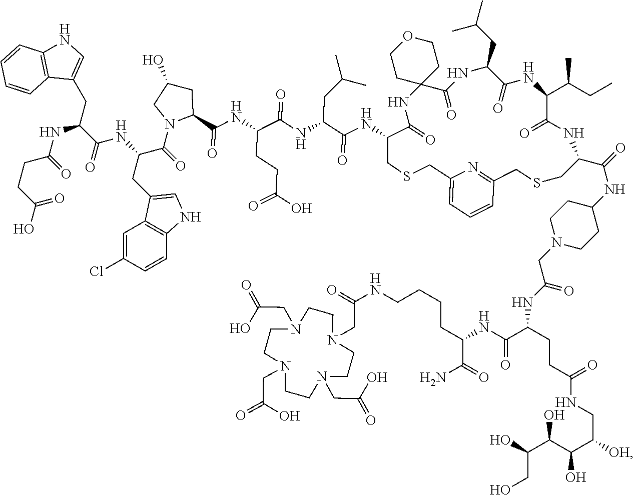 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)CCC(=O)O)CSCc2cccc(n2)CSC[C@@H](C(=O)NC2CCN(CC(=O)N[C@H](CCC(=O)NC[C@H](O)[C@@H](O)[C@H](O)[C@H](O)CO)C(=O)N[C@@H](CCCCNC(=O)CN3CCN(CC(=O)O)CCN(CC(=O)O)CCN(CC(=O)O)CC3)C(N)=O)CC2)NC1=O]

compound HO-Succinyl-Trp-5Clw-Tap-Glu-leu-[Cys(2Lut)-Apc-Leu-Ile-Cys]-APAc-glu(AGLU)-Lys(DOTA)-NH₂ (GIP-022, SEQ ID NO: 26) of the following formula

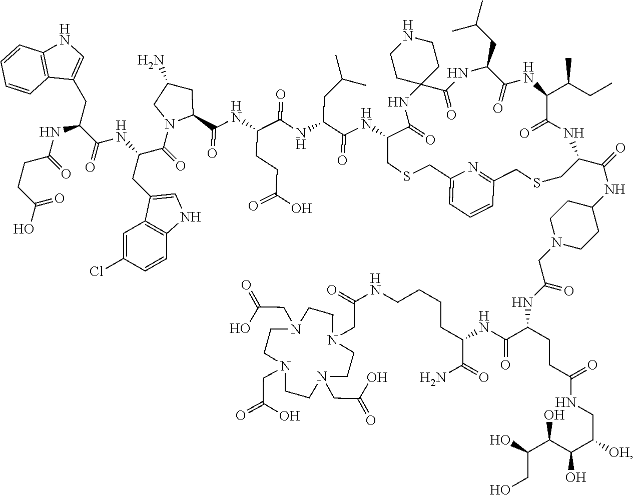 [SMILES: CC[C@H](C)[C@@H]1NC(=O)[C@H](CC(C)C)NC(=O)C2(CCNCC2)NC(=O)[C@@H](NC(=O)[C@@H](CC(C)C)NC(=O)[C@H](CCC(=O)O)NC(=O)[C@@H]2C[C@@H](N)CN2C(=O)[C@H](Cc2c[nH]c3ccc(Cl)cc23)NC(=O)[C@H](Cc2c[nH]c3ccccc23)NC(=O)CCC(=O)O)CSCc2cccc(n2)CSC[C@@H](C(=O)NC2CCN(CC(=O)N[C@H](CCC(=O)NC[C@H](O)[C@@H](O)[C@H](O)[C@H](O)CO)C(=O)N[C@@H](CCCCNC(=O)CN3CCN(CC(=O)O)CCN(CC(=O)O)CCN(CC(=O)O)CC3)C(N)=O)CC2)NC1=O]

compound HO-Succinyl-Trp-5Clw-Tap-Glu-leu-[Cys(2Lut)-Apc(DOTA)-Leu-Ile-Cys]-APAc-glu(AGLU)-NH₂ (GIP-023, SEQ ID NO: 27) of the following formula

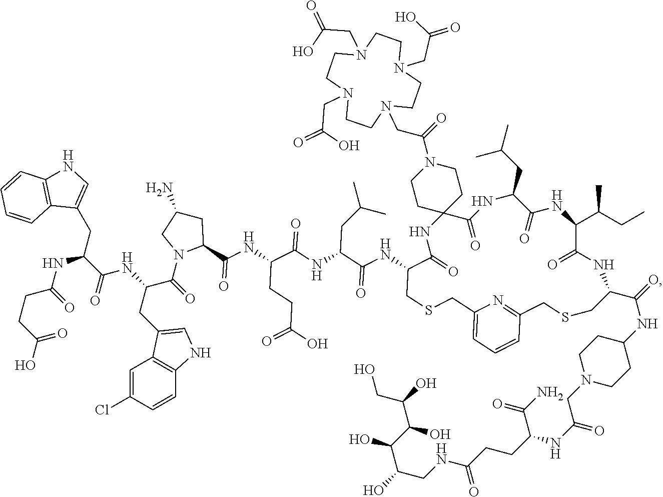 [SMILES: CC[C@H](C)[C@@H]1NC(=O)[C@H](CC(C)C)NC(=O)C2(CCN(C(=O)CN3CCN(CC(=O)O)CCN(CC(=O)O)CCN(CC(=O)O)CC3)CC2)NC(=O)[C@@H](NC(=O)[C@@H](CC(C)C)NC(=O)[C@H](CCC(=O)O)NC(=O)[C@@H]2C[C@@H](N)CN2C(=O)[C@H](Cc2c[nH]c3ccc(Cl)cc23)NC(=O)[C@H](Cc2c[nH]c3ccccc23)NC(=O)CCC(=O)O)CSCc2cccc(n2)CSC[C@@H](C(=O)NC2CCN(CC(=O)N[C@H](CCC(=O)NC[C@H](O)[C@@H](O)[C@H](O)[C@H](O)CO)C(N)=O)CC2)NC1=O]

compound AGLU-Glutar-Asp-Trp-5Clw-Tap-Glu-leu-[Cys(2Lut)-Lys(DOTA)-Leu-Ile-Cys]-APAc-glu(AGLU)-NH₂ (GIP-024, SEQ ID NO: 28) of the following formula

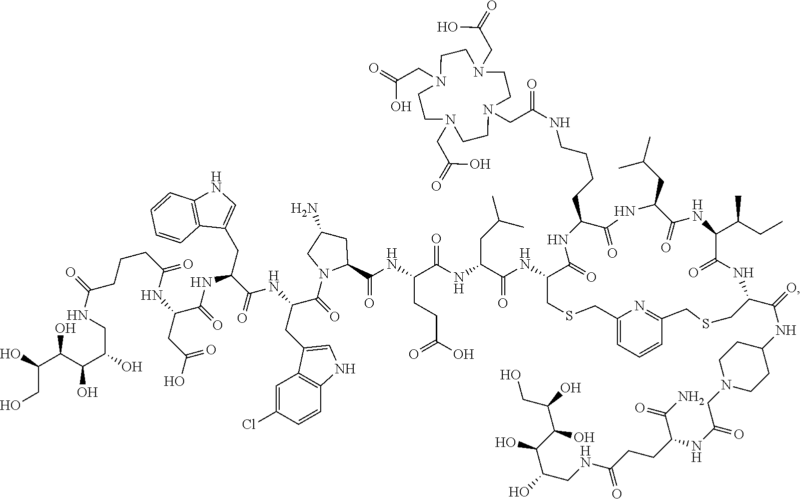 [SMILES: CC[C@H](C)[C@@H]1NC(=O)[C@H](CC(C)C)NC(=O)[C@H](CCCCNC(=O)CN2CCN(CC(=O)O)CCN(CC(=O)O)CCN(CC(=O)O)CC2)NC(=O)[C@@H](NC(=O)[C@@H](CC(C)C)NC(=O)[C@H](CCC(=O)O)NC(=O)[C@@H]2C[C@@H](N)CN2C(=O)[C@H](Cc2c[nH]c3ccc(Cl)cc23)NC(=O)[C@H](Cc2c[nH]c3ccccc23)NC(=O)[C@H](CC(=O)O)NC(=O)CCCC(=O)NC[C@H](O)[C@@H](O)[C@H](O)[C@H](O)CO)CSCc2cccc(n2)CSC[C@@H](C(=O)NC2CCN(CC(=O)N[C@H](CCC(=O)NC[C@H](O)[C@@H](O)[C@H](O)[C@H](O)CO)C(N)=O)CC2)NC1=O]

compound AGLU-Glutar-Asp-Trp-5Clw-Tap-Glu-leu-[Cys(2Lut)-lys(DOTA)-Leu-Ile-Cys]-APAc-glu(AGLU)-NH₂ (GIP-025, SEQ ID NO: 29) of the following formula

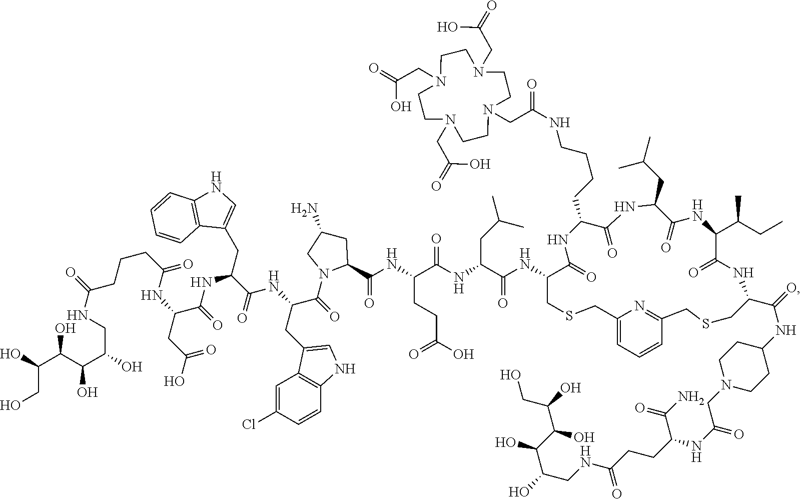 [SMILES: CC[C@H](C)[C@@H]1NC(=O)[C@H](CC(C)C)NC(=O)[C@@H](CCCCNC(=O)CN2CCN(CC(=O)O)CCN(CC(=O)O)CCN(CC(=O)O)CC2)NC(=O)[C@@H](NC(=O)[C@@H](CC(C)C)NC(=O)[C@H](CCC(=O)O)NC(=O)[C@@H]2C[C@@H](N)CN2C(=O)[C@H](Cc2c[nH]c3ccc(Cl)cc23)NC(=O)[C@H](Cc2c[nH]c3ccccc23)NC(=O)[C@H](CC(=O)O)NC(=O)CCCC(=O)NC[C@H](O)[C@@H](O)[C@H](O)[C@H](O)CO)CSCc2cccc(n2)CSC[C@@H](C(=O)NC2CCN(CC(=O)N[C@H](CCC(=O)NC[C@H](O)[C@@H](O)[C@H](O)[C@H](O)CO)C(N)=O)CC2)NC1=O]

compound AGLU-Glutar-Asp-Trp-5Clw-Tap-Glu-leu-[Cys(2Lut)-Dab(DOTA)-Leu-Ile-Cys]-APAc-glu(AGLU)-NH₂ (GIP-026, SEQ ID NO: 30) of the following formula

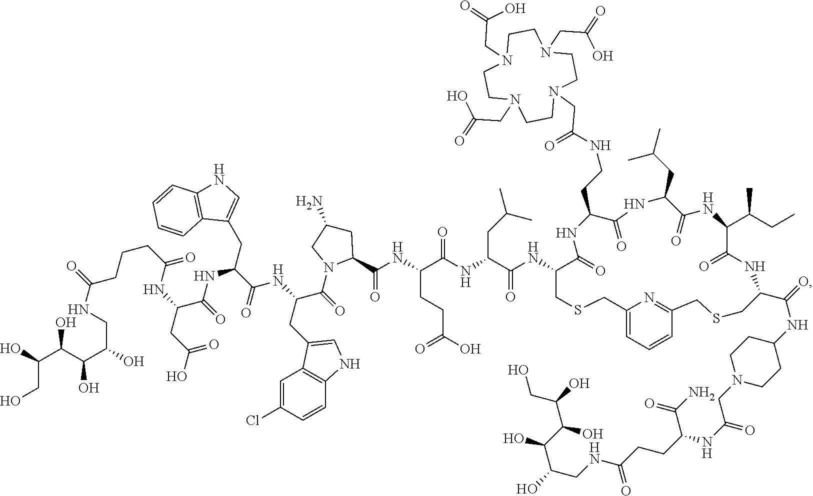 [SMILES: CC[C@H](C)[C@@H]1NC(=O)[C@H](CC(C)C)NC(=O)[C@H](CCNC(=O)CN2CCN(CC(=O)O)CCN(CC(=O)O)CCN(CC(=O)O)CC2)NC(=O)[C@@H](NC(=O)[C@@H](CC(C)C)NC(=O)[C@H](CCC(=O)O)NC(=O)[C@@H]2C[C@@H](N)CN2C(=O)[C@H](Cc2c[nH]c3ccc(Cl)cc23)NC(=O)[C@H](Cc2c[nH]c3ccccc23)NC(=O)[C@H](CC(=O)O)NC(=O)CCCC(=O)NC[C@H](O)[C@@H](O)[C@H](O)[C@H](O)CO)CSCc2cccc(n2)CSC[C@@H](C(=O)NC2CCN(CC(=O)N[C@H](CCC(=O)NC[C@H](O)[C@@H](O)[C@H](O)[C@H](O)CO)C(N)=O)CC2)NC1=O]

compound AGLU-Glutar-Asp-Trp-5Clw-Tap-Glu-leu-[Cys(2Lut)-dab(DOTA)-Leu-Ile-Cys]-APAc-glu(AGLU)-NH₂ (GIP-027, SEQ ID NO: 31) of the following formula

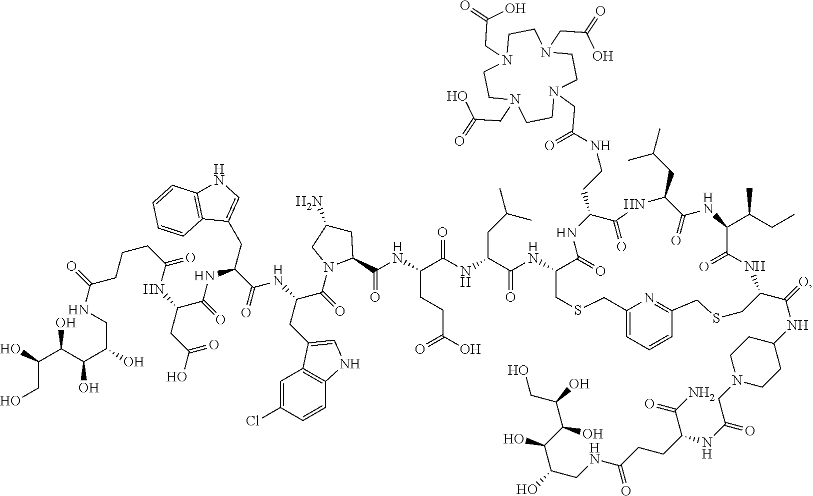 [SMILES: CC[C@H](C)[C@@H]1NC(=O)[C@H](CC(C)C)NC(=O)[C@@H](CCNC(=O)CN2CCN(CC(=O)O)CCN(CC(=O)O)CCN(CC(=O)O)CC2)NC(=O)[C@@H](NC(=O)[C@@H](CC(C)C)NC(=O)[C@H](CCC(=O)O)NC(=O)[C@@H]2C[C@@H](N)CN2C(=O)[C@H](Cc2c[nH]c3ccc(Cl)cc23)NC(=O)[C@H](Cc2c[nH]c3ccccc23)NC(=O)[C@H](CC(=O)O)NC(=O)CCCC(=O)NC[C@H](O)[C@@H](O)[C@H](O)[C@H](O)CO)CSCc2cccc(n2)CSC[C@@H](C(=O)NC2CCN(CC(=O)N[C@H](CCC(=O)NC[C@H](O)[C@@H](O)[C@H](O)[C@H](O)CO)C(N)=O)CC2)NC1=O]

compound AGLU-Glutar-Asp-Trp-5Clw-Tap-Glu-leu-[Cys(2Lut)-Apc(NODAGA)-Leu-Ile-Cys]-APAc-glu(AGLU)-NH₂ (GIP-028, SEQ ID NO: 32) of the following formula

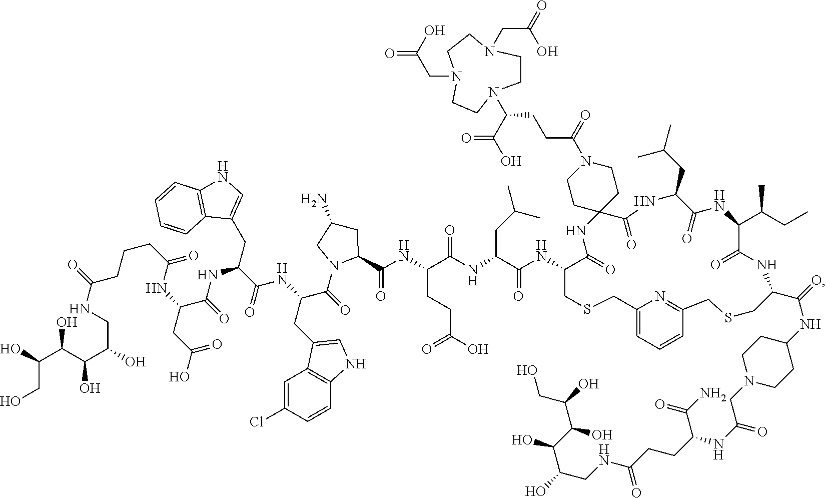 [SMILES: CC[C@H](C)[C@@H]1NC(=O)[C@H](CC(C)C)NC(=O)C2(CCN(C(=O)CC[C@H](C(=O)O)N3CCN(CC(=O)O)CCN(CC(=O)O)CC3)CC2)NC(=O)[C@@H](NC(=O)[C@@H](CC(C)C)NC(=O)[C@H](CCC(=O)O)NC(=O)[C@@H]2C[C@@H](N)CN2C(=O)[C@H](Cc2c[nH]c3ccc(Cl)cc23)NC(=O)[C@H](Cc2c[nH]c3ccccc23)NC(=O)[C@H](CC(=O)O)NC(=O)CCCC(=O)NC[C@H](O)[C@@H](O)[C@H](O)[C@H](O)CO)CSCc2cccc(n2)CSC[C@@H](C(=O)NC2CCN(CC(=O)N[C@H](CCC(=O)NC[C@H](O)[C@@H](O)[C@H](O)[C@H](O)CO)C(N)=O)CC2)NC1=O]

compound AGLU-Glutar-Asp-Trp-5Clw-Tap-Glu-leu-[Cys(2Lut)-Apc(NOPO)-Leu-Ile-Cys]-APAc-glu(AGLU)-NH₂ (GIP-029, SEQ ID NO: 33) of the following formula

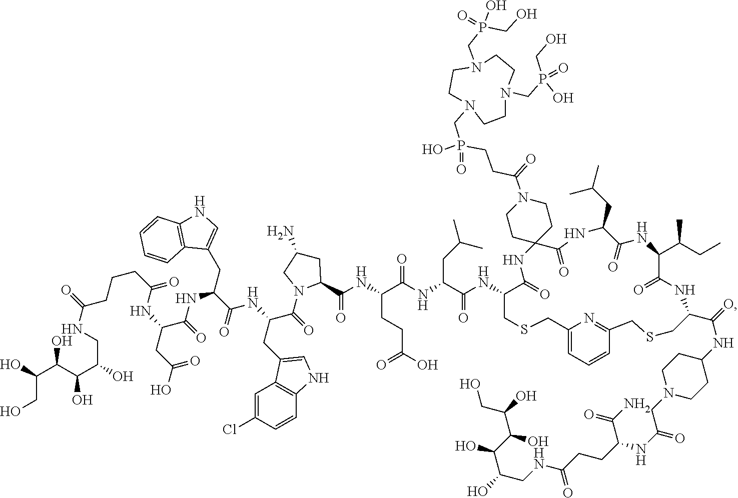 [SMILES: CC[C@H](C)[C@@H]1NC(=O)[C@H](CC(C)C)NC(=O)C2(CCN(C(=O)CCP(=O)(O)CN3CCN(CP(=O)(O)CO)CCN(CP(=O)(O)CO)CC3)CC2)NC(=O)[C@@H](NC(=O)[C@@H](CC(C)C)NC(=O)[C@H](CCC(=O)O)NC(=O)[C@@H]2C[C@@H](N)CN2C(=O)[C@H](Cc2c[nH]c3ccc(Cl)cc23)NC(=O)[C@H](Cc2c[nH]c3ccccc23)NC(=O)[C@H](CC(=O)O)NC(=O)CCCC(=O)NC[C@H](O)[C@@H](O)[C@H](O)[C@H](O)CO)CSCc2cccc(n2)CSC[C@@H](C(=O)NC2CCN(CC(=O)N[C@H](CCC(=O)NC[C@H](O)[C@@H](O)[C@H](O)[C@H](O)CO)C(N)=O)CC2)NC1=O]

compound AGLU-Glutar-Asp-Trp-5Clw-Tap-Glu-leu-[Cys(2Lut)-Apc(N4Ac)-Leu-Ile-Cys]-APAc-glu(AGLU)-NH₂ (GIP-030, SEQ ID NO: 34) of the following formula

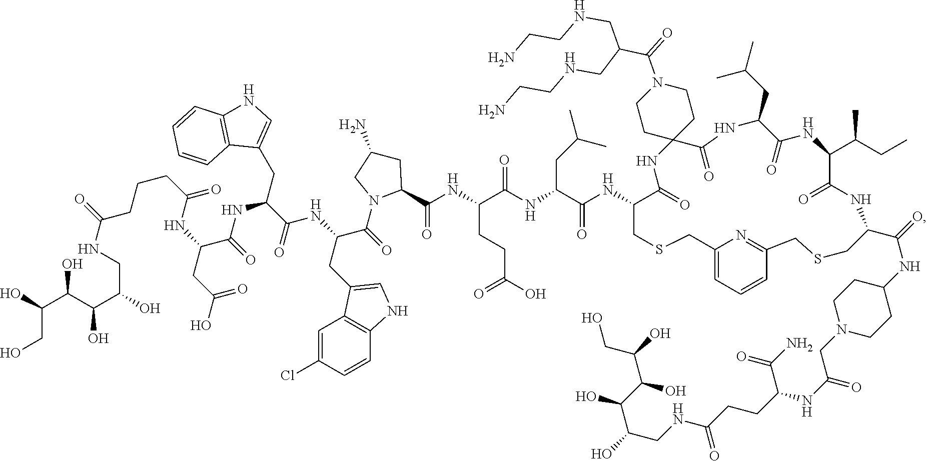 [SMILES: CC[C@H](C)[C@@H]1NC(=O)[C@H](CC(C)C)NC(=O)C2(CCN(C(=O)C(CNCCN)CNCCN)CC2)NC(=O)[C@@H](NC(=O)[C@@H](CC(C)C)NC(=O)[C@H](CCC(=O)O)NC(=O)[C@@H]2C[C@@H](N)CN2C(=O)[C@H](Cc2c[nH]c3ccc(Cl)cc23)NC(=O)[C@H](Cc2c[nH]c3ccccc23)NC(=O)[C@H](CC(=O)O)NC(=O)CCCC(=O)NC[C@H](O)[C@@H](O)[C@H](O)[C@H](O)CO)CSCc2cccc(n2)CSC[C@@H](C(=O)NC2CCN(CC(=O)N[C@H](CCC(=O)NC[C@H](O)[C@@H](O)[C@H](O)[C@H](O)CO)C(N)=O)CC2)NC1=O]

compound AGLU-Glutar-Asp-Trp-5Clw-Tap-Glu-leu-[Cys(2Lut)-Apc(NODAGA-Gly)-Leu-Ile-Cys]-APAc-glu(AGLU)-NH₂ (GIP-031, SEQ ID NO: 35) of the following formula

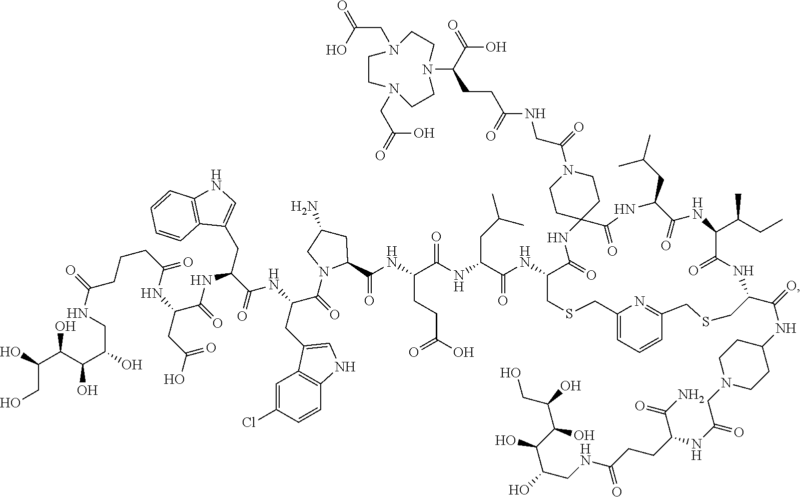 [SMILES: CC[C@H](C)[C@@H]1NC(=O)[C@H](CC(C)C)NC(=O)C2(CCN(C(=O)CNC(=O)CC[C@H](C(=O)O)N3CCN(CC(=O)O)CCN(CC(=O)O)CC3)CC2)NC(=O)[C@@H](NC(=O)[C@@H](CC(C)C)NC(=O)[C@H](CCC(=O)O)NC(=O)[C@@H]2C[C@@H](N)CN2C(=O)[C@H](Cc2c[nH]c3ccc(Cl)cc23)NC(=O)[C@H](Cc2c[nH]c3ccccc23)NC(=O)[C@H](CC(=O)O)NC(=O)CCCC(=O)NC[C@H](O)[C@@H](O)[C@H](O)[C@H](O)CO)CSCc2cccc(n2)CSC[C@@H](C(=O)NC2CCN(CC(=O)N[C@H](CCC(=O)NC[C@H](O)[C@@H](O)[C@H](O)[C@H](O)CO)C(N)=O)CC2)NC1=O]

compound AGLU-Glutar-APAc-Asp-Trp-5Clw-Hyp-Glu-leu-[Cys(2Lut)-Apc(DOTA)-Leu-Ile-Cys]-glu(AGLU)-NH₂ (GIP-033, SEQ ID NO: 36) of the following formula

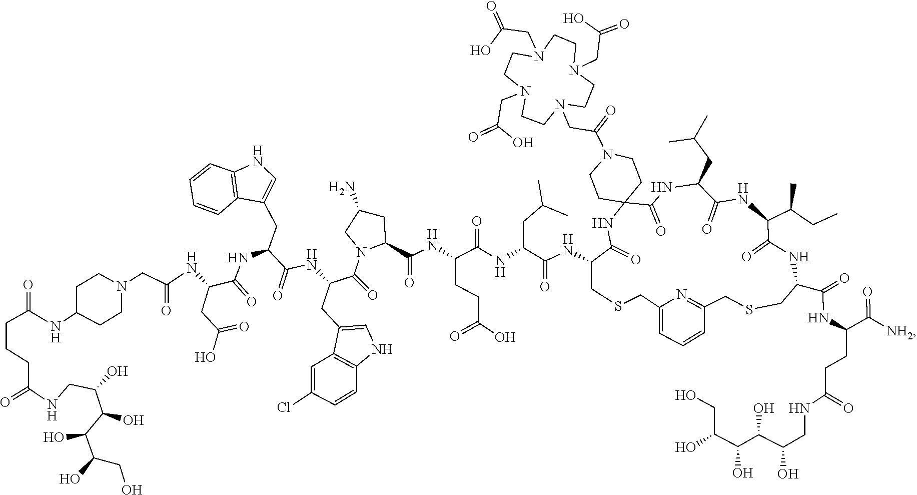 [SMILES: CC[C@H](C)[C@@H]1NC(=O)[C@H](CC(C)C)NC(=O)C2(CCN(C(=O)CN3CCN(CC(=O)O)CCN(CC(=O)O)CCN(CC(=O)O)CC3)CC2)NC(=O)[C@@H](NC(=O)[C@@H](CC(C)C)NC(=O)[C@H](CCC(=O)O)NC(=O)[C@@H]2C[C@@H](N)CN2C(=O)[C@H](Cc2c[nH]c3ccc(Cl)cc23)NC(=O)[C@H](Cc2c[nH]c3ccccc23)NC(=O)[C@H](CC(=O)O)NC(=O)CN2CCC(NC(=O)CCCC(=O)NC[C@H](O)[C@@H](O)[C@H](O)[C@H](O)CO)CC2)CSCc2cccc(n2)CSC[C@@H](C(=O)N[C@H](CCC(=O)NC[C@H](O)[C@@H](O)[C@H](O)[C@H](O)CO)C(N)=O)NC1=O]

compound AGLU-Glutar-Hse-Trp-5Clw-Tap-Glu-leu-[Cys(2Lut)-Apc(DOTA)-Leu-Ile-Cys]-APAc-glu(AGLU)-NH₂ (GIP-034, SEQ ID NO: 37) of the following formula

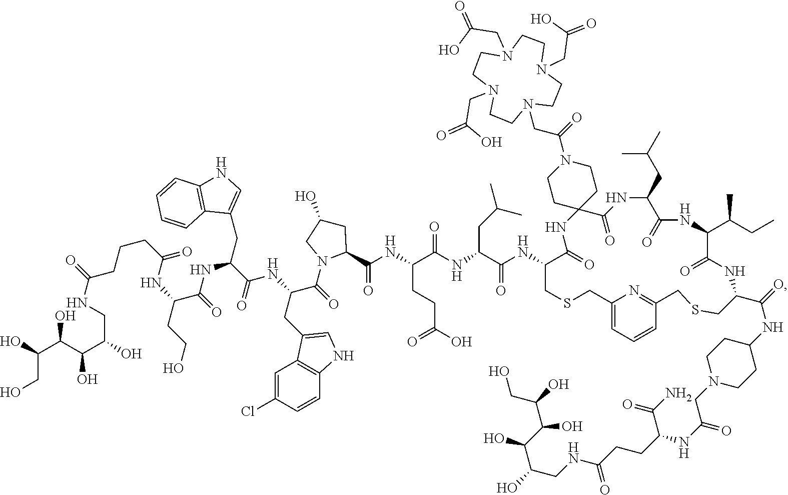

compound AGLU-Glutar-Asp-Trp-5Clw-Tap-Glu-Leu-[Cys(2Lut)-Apc(DOTA)-Leu-Ile-Cys]-APAc-glu(AGLU)-NH₂ (GIP-035, SEQ ID NO: 38) of the following formula

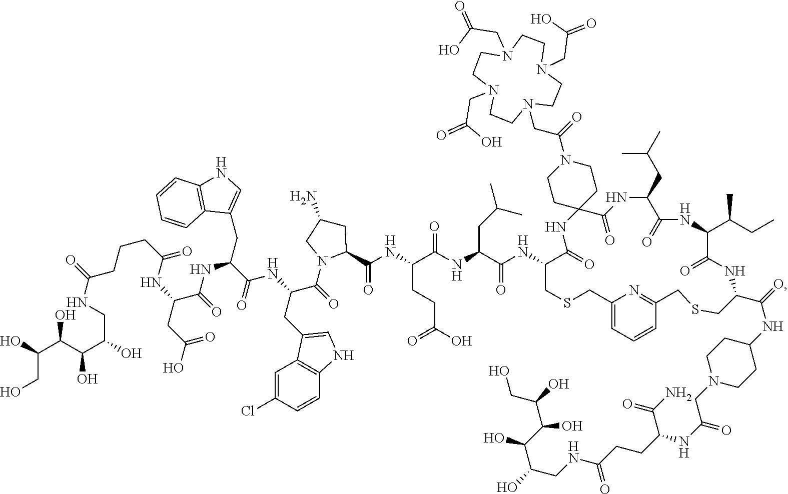

compound DOTA-glu(AGLU)-Asp-Trp-5Clw-Hyp-Glu-leu-[Cys(2Lut)-Thp-Leu-Ile-Cys]-APAc-glu(AGLU)-NH₂ (GIP-036, SEQ ID NO: 39) of the following formula

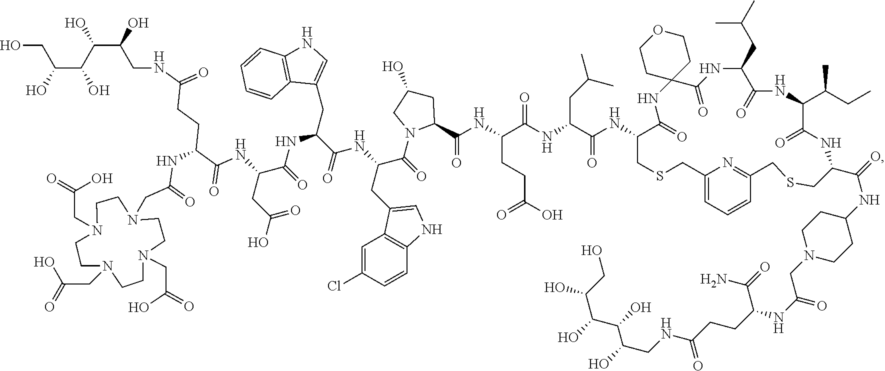 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CC(=O)O)NC(=O)[C@@H](CCC(=O)NC[C@H](O)[C@@H](O)[C@H](O)[C@H](O)CO)NC(=O)CN2CCN(CC(=O)O)CCN(CC(=O)O)CCN(CC(=O)O)CC2)CSCc2cccc(n2)CSC[C@@H](C(=O)NC2CCN(CC(=O)N[C@H](CCC(=O)NC[C@H](O)[C@@H](O)[C@H](O)[C@H](O)CO)C(N)=O)CC2)NC1=O]

Compound DOTA-PEG6-glu(AGLU)-Asp-Trp-5Clw-Tap-Glu-leu-[Cys(2Lut)-Thp-Leu-Ile-Cys]-APAc-glu(AGLU)-NH₂ (GIP-037, SEQ ID NO: 40) of the following formula

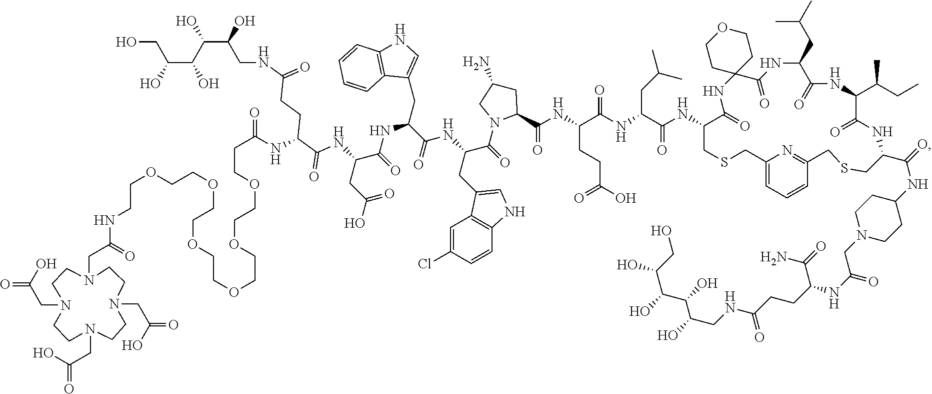 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](N)CN2C(=O)[C@H](Cc2c[nH]c3ccc(Cl)cc23)NC(=O)[C@H](Cc2c[nH]c3ccccc23)NC(=O)[C@H](CC(=O)O)NC(=O)[C@@H](CCC(=O)NC[C@H](O)[C@@H](O)[C@H](O)[C@H](O)CO)NC(=O)CCOCCOCCOCCOCCOCCOCCNC(=O)CN2CCN(CC(=O)O)CCN(CC(=O)O)CCN(CC(=O)O)CC2)CSCc2cccc(n2)CSC[C@@H](C(=O)NC2CCN(CC(=O)N[C@H](CCC(=O)NC[C@H](O)[C@@H](O)[C@H](O)[C@H](O)CO)C(N)=O)CC2)NC1=O]

compound AGLU-Glutar-Asp-Trp-5Clw-Tap-Glu-leu-[Cys(2Lut)-Apc(DOTA)-Leu-Ile-cys]-APAc-glu(AGLU)-NH₂ (GIP-038, SEQ ID NO: 41) of the following formula

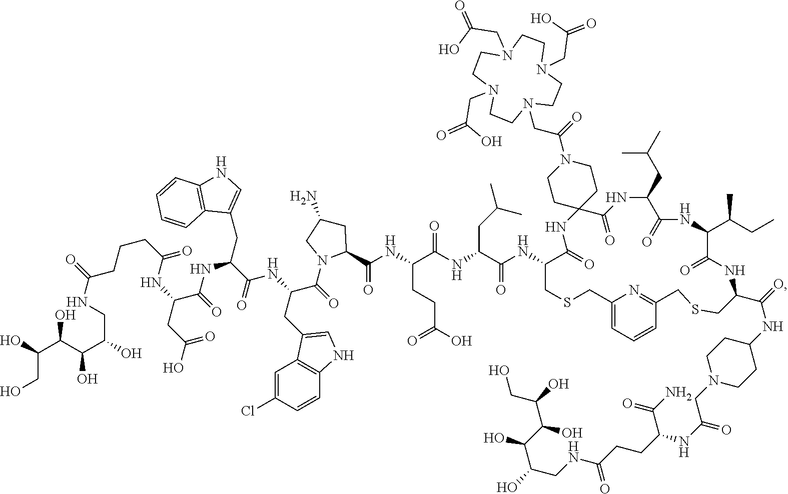 [SMILES: CC[C@H](C)[C@@H]1NC(=O)[C@H](CC(C)C)NC(=O)C2(CCN(C(=O)CN3CCN(CC(=O)O)CCN(CC(=O)O)CCN(CC(=O)O)CC3)CC2)NC(=O)[C@@H](NC(=O)[C@@H](CC(C)C)NC(=O)[C@H](CCC(=O)O)NC(=O)[C@@H]2C[C@@H](N)CN2C(=O)[C@H](Cc2c[nH]c3ccc(Cl)cc23)NC(=O)[C@H](Cc2c[nH]c3ccccc23)NC(=O)[C@H](CC(=O)O)NC(=O)CCCC(=O)NC[C@H](O)[C@@H](O)[C@H](O)[C@H](O)CO)CSCc2cccc(n2)CSC[C@H](C(=O)NC2CCN(CC(=O)N[C@H](CCC(=O)NC[C@H](O)[C@@H](O)[C@H](O)[C@H](O)CO)C(N)=O)CC2)NC1=O]

compound AGLU-Glutar-APAc-Asp-Trp-5Clw-Hyp-Glu-leu-[Cys(2Lut)-Apc(DOTA)-Leu-Ile-Cys]-lys(MeO36PEG)-NH₂ (GIP-039, SEQ ID NO: 42) of the following formula

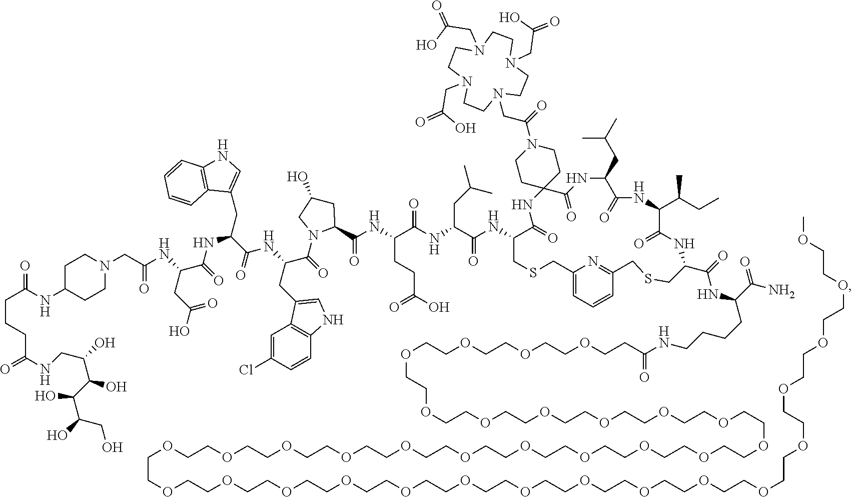

compound Me036PEG-APAc-Asp-Trp-5Clw-Hyp-Glu-leu-[Cys(2Lut)-Apc(DOTA)-Leu-Ile-Cys]-glu(AGLU)-NH₂ (GIP-040, SEQ ID NO: 43) of the following formula

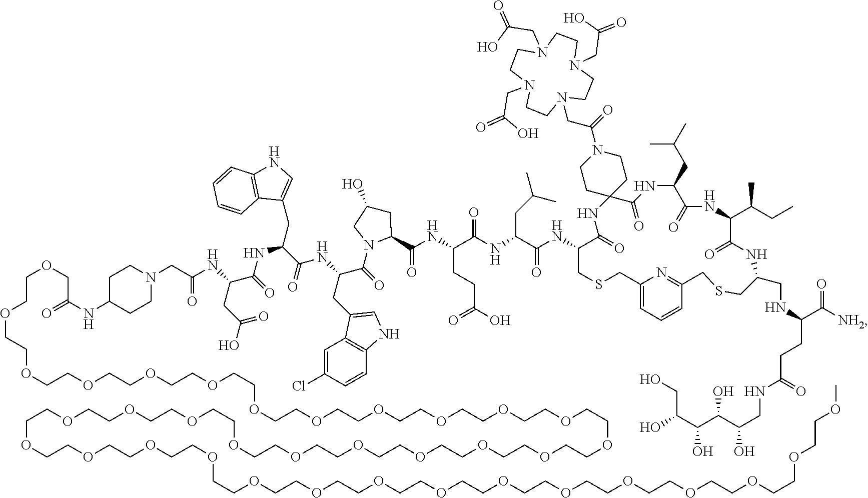 [SMILES: CC[C@H](C)[C@@H]1NC(=O)[C@H](CC(C)C)NC(=O)C2(CCN(C(=O)CN3CCN(CC(=O)O)CCN(CC(=O)O)CCN(CC(=O)O)CC3)CC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CC(=O)O)NC(=O)CN2CCC(NC(=O)COCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOC)CC2)CSCc2cccc(n2)CSC[C@@H](CN[C@H](CCC(=O)NC[C@H](O)[C@@H](O)[C@H](O)[C@H](O)CO)C(N)=O)NC1=O]

compound MeO36PEG-APAc-Asp-Trp-5Clw-Hyp-Glu-leu-[Cys(2Lut)-Apc(DOTA)-Leu-Ile-Cys]-APAc-glu(AGLU)-NH₂ (GIP-041, SEQ ID NO: 44) of the following formula

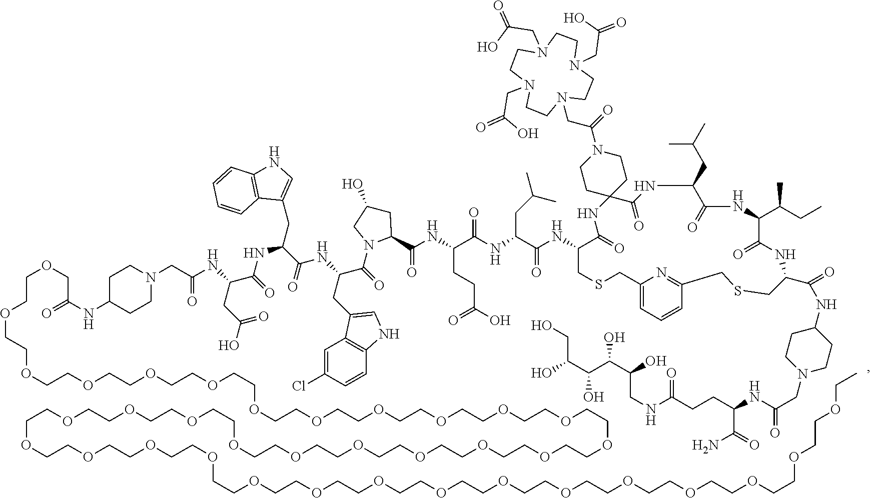

compound MeO36PEG-APAc-Asp-Trp-5Clw-Tap-Glu-leu-[Cys(2Lut)-Apc(DOTA)-Leu-Ile-Cys]-APAc-glu(AGLU)-NH₂ (GIP-042, SEQ ID NO: 45) of the following formula

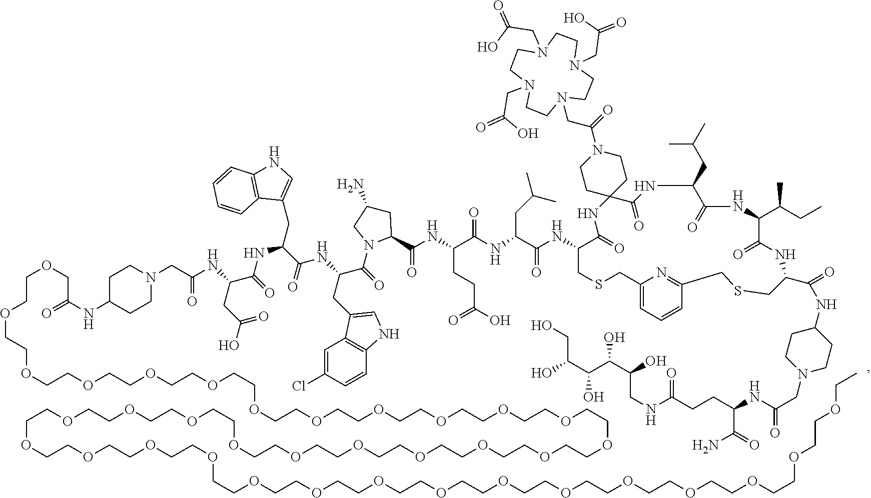

compound AGLU-Glutar-APAc-Asp-Trp-5Clw-Hyp-Glu-leu-[Cys(2Lut)-Apc(DOTA-glu(AGLU))-Leu-Ile-Cys]-APAc-glu(AGLU)-NH₂ (GIP-043, SEQ ID NO: 46) of the following formula

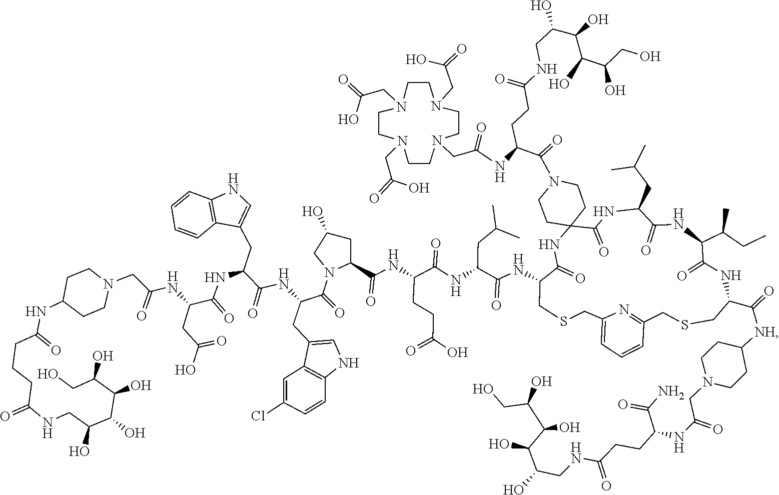 [SMILES: CC[C@H](C)[C@@H]1NC(=O)[C@H](CC(C)C)NC(=O)C2(CCN(C(=O)[C@H](CCC(=O)NC[C@H](O)[C@@H](O)[C@H](O)[C@H](O)CO)NC(=O)CN3CCN(CC(=O)O)CCN(CC(=O)O)CCN(CC(=O)O)CC3)CC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CC(=O)O)NC(=O)CN2CCC(NC(=O)CCCC(=O)NC[C@H](O)[C@@H](O)[C@H](O)[C@H](O)CO)CC2)CSCc2cccc(n2)CSC[C@@H](C(=O)NC2CCN(CC(=O)N[C@H](CCC(=O)NC[C@H](O)[C@@H](O)[C@H](O)[C@H](O)CO)C(N)=O)CC2)NC1=O]

compound DOTA-02Oc-glu(AGLU)-Asp-Trp-5Clw-Tap-Glu-leu-[Cys(2Lut)-Apc(AGLU-Glutar)-Leu-Ile-Cys]-APAc-glu(AGLU)-NH₂ (GIP-044, SEQ ID NO: 47) of the following formula

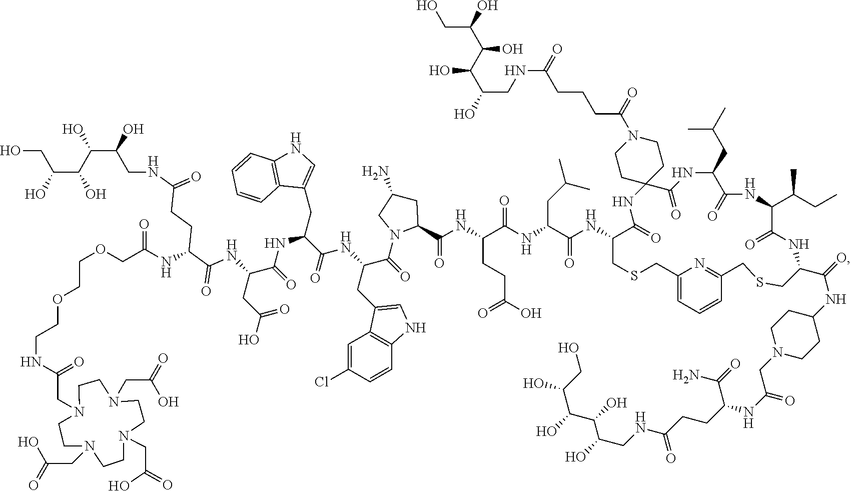 [SMILES: CC[C@H](C)[C@@H]1NC(=O)[C@H](CC(C)C)NC(=O)C2(CCN(C(=O)CCCC(=O)NC[C@H](O)[C@@H](O)[C@H](O)[C@H](O)CO)CC2)NC(=O)[C@@H](NC(=O)[C@@H](CC(C)C)NC(=O)[C@H](CCC(=O)O)NC(=O)[C@@H]2C[C@@H](N)CN2C(=O)[C@H](Cc2c[nH]c3ccc(Cl)cc23)NC(=O)[C@H](Cc2c[nH]c3ccccc23)NC(=O)[C@H](CC(=O)O)NC(=O)[C@@H](CCC(=O)NC[C@H](O)[C@@H](O)[C@H](O)[C@H](O)CO)NC(=O)COCCOCCNC(=O)CN2CCN(CC(=O)O)CCN(CC(=O)O)CCN(CC(=O)O)CC2)CSCc2cccc(n2)CSC[C@@H](C(=O)NC2CCN(CC(=O)N[C@H](CCC(=O)NC[C@H](O)[C@@H](O)[C@H](O)[C@H](O)CO)C(N)=O)CC2)NC1=O]

compound DOTA-glu(AGLU)-Asp-Trp-5Clw-Tap-Glu-leu-[Cys(2Lut)-Apc(AGLU-Glutar)-Leu-Ile-Cys]-APAc-glu(AGLU)-NH₂ (GIP-045, SEQ ID NO: 48) of the following formula

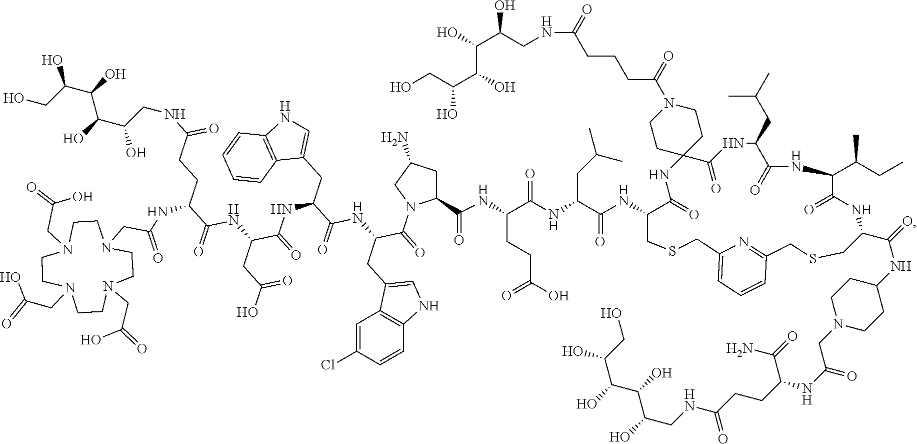 [SMILES: CC[C@H](C)[C@@H]1NC(=O)[C@H](CC(C)C)NC(=O)C2(CCN(C(=O)CCCC(=O)NC[C@H](O)[C@@H](O)[C@H](O)[C@H](O)CO)CC2)NC(=O)[C@@H](NC(=O)[C@@H](CC(C)C)NC(=O)[C@H](CCC(=O)O)NC(=O)[C@@H]2C[C@@H](N)CN2C(=O)[C@H](Cc2c[nH]c3ccc(Cl)cc23)NC(=O)[C@H](Cc2c[nH]c3ccccc23)NC(=O)[C@H](CC(=O)O)NC(=O)[C@@H](CCC(=O)NC[C@H](O)[C@@H](O)[C@H](O)[C@H](O)CO)NC(=O)CN2CCN(CC(=O)O)CCN(CC(=O)O)CCN(CC(=O)O)CC2)CSCc2cccc(n2)CSC[C@@H](C(=O)NC2CCN(CC(=O)N[C@H](CCC(=O)NC[C@H](O)[C@@H](O)[C@H](O)[C@H](O)CO)C(N)=O)CC2)NC1=O]

compound AGLU-Glutar-APAc-Ser-Trp-5Clw-Tap(AGLU-Glutar)-Hse-leu-[Cys(2Lut)-Apc(DOTA)-Leu-Ile-Cys]-APAc-glu(AGLU)-NH₂ (GIP-046, SEQ ID NO: 49) of the following formula

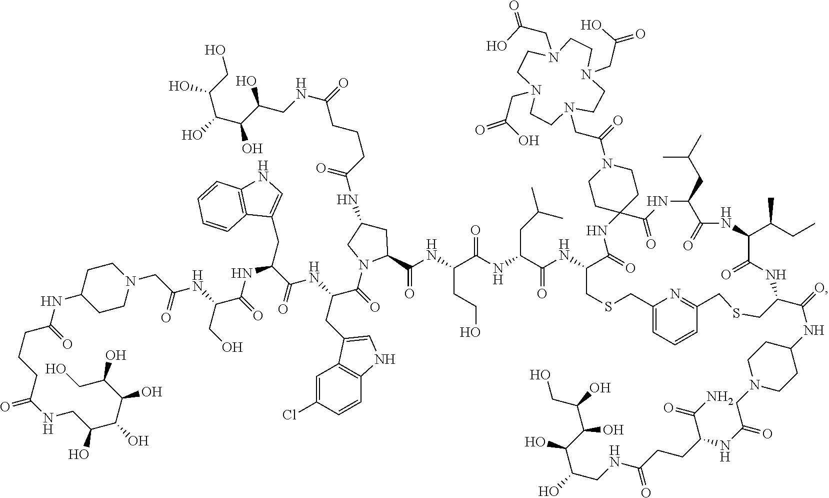 [SMILES: CC[C@H](C)[C@@H]1NC(=O)[C@H](CC(C)C)NC(=O)C2(CCN(C(=O)CN3CCN(CC(=O)O)CCN(CC(=O)O)CCN(CC(=O)O)CC3)CC2)NC(=O)[C@@H](NC(=O)[C@@H](CC(C)C)NC(=O)[C@H](CCO)NC(=O)[C@@H]2C[C@@H](NC(=O)CCCC(=O)NC[C@H](O)[C@@H](O)[C@H](O)[C@H](O)CO)CN2C(=O)[C@H](Cc2c[nH]c3ccc(Cl)cc23)NC(=O)[C@H](Cc2c[nH]c3ccccc23)NC(=O)[C@H](CO)NC(=O)CN2CCC(NC(=O)CCCC(=O)NC[C@H](O)[C@@H](O)[C@H](O)[C@H](O)CO)CC2)CSCc2cccc(n2)CSC[C@@H](C(=O)NC2CCN(CC(=O)N[C@H](CCC(=O)NC[C@H](O)[C@@H](O)[C@H](O)[C@H](O)CO)C(N)=O)CC2)NC1=O]

compound AGLU-Glutar-APAc-Hse-Trp-5Clw-Hyp-Glu-leu-[Cys(2Lut)-Apc(DOTA-glu(AGLU))-Leu-Ile-Cys]-APAc-glu(AGLU)-NH₂ (GIP-047, SEQ ID NO: 50) of the following formula

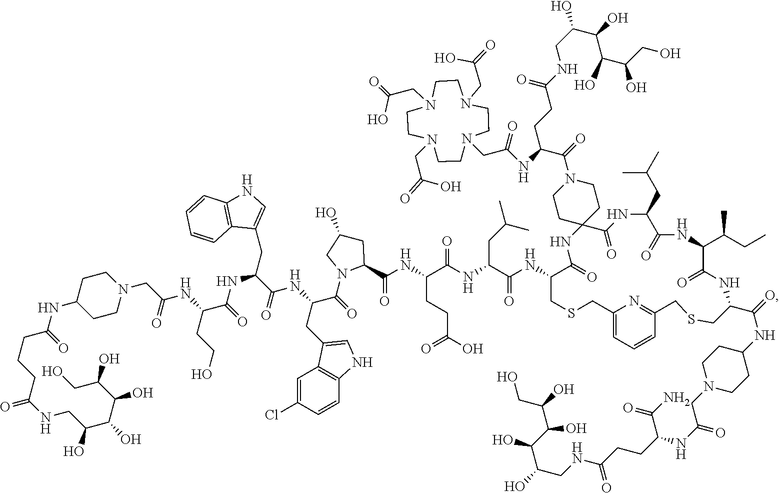 [SMILES: CC[C@H](C)[C@@H]1NC(=O)[C@H](CC(C)C)NC(=O)C2(CCN(C(=O)[C@H](CCC(=O)NC[C@H](O)[C@@H](O)[C@H](O)[C@H](O)CO)NC(=O)CN3CCN(CC(=O)O)CCN(CC(=O)O)CCN(CC(=O)O)CC3)CC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CCO)NC(=O)CN2CCC(NC(=O)CCCC(=O)NC[C@H](O)[C@@H](O)[C@H](O)[C@H](O)CO)CC2)CSCc2cccc(n2)CSC[C@@H](C(=O)NC2CCN(CC(=O)N[C@H](CCC(=O)NC[C@H](O)[C@@H](O)[C@H](O)[C@H](O)CO)C(N)=O)CC2)NC1=O]

compound AGLU-Glutar-APAc-Ser-Trp-5Clw-Tap-Hse-leu-[Cys(2Lut)-Apc(DOTA)-Leu-Ile-Cys]-APAc-glu(AGLU)-NH₂ (GIP-048, SEQ ID NO: 51) of the following formula

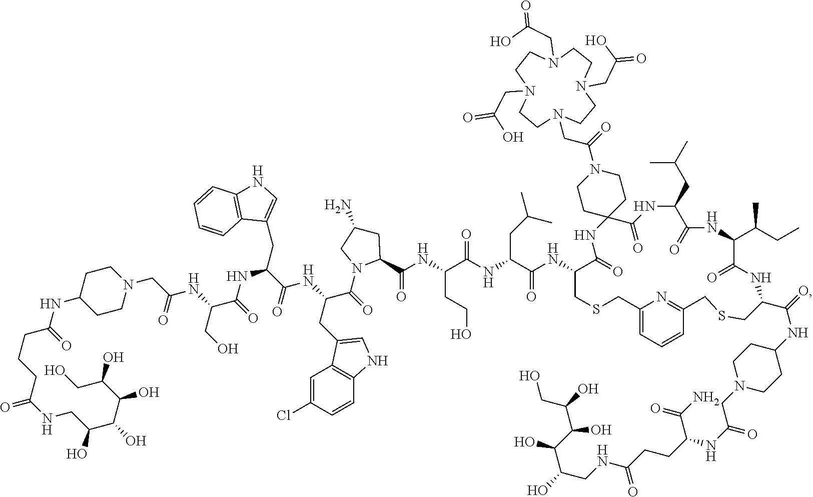 [SMILES: CC[C@H](C)[C@@H]1NC(=O)[C@H](CC(C)C)NC(=O)C2(CCN(C(=O)CN3CCN(CC(=O)O)CCN(CC(=O)O)CCN(CC(=O)O)CC3)CC2)NC(=O)[C@@H](NC(=O)[C@@H](CC(C)C)NC(=O)[C@H](CCO)NC(=O)[C@@H]2C[C@@H](N)CN2C(=O)[C@H](Cc2c[nH]c3ccc(Cl)cc23)NC(=O)[C@H](Cc2c[nH]c3ccccc23)NC(=O)[C@H](CO)NC(=O)CN2CCC(NC(=O)CCCC(=O)NC[C@H](O)[C@@H](O)[C@H](O)[C@H](O)CO)CC2)CSCc2cccc(n2)CSC[C@@H](C(=O)NC2CCN(CC(=O)N[C@H](CCC(=O)NC[C@H](O)[C@@H](O)[C@H](O)[C@H](O)CO)C(N)=O)CC2)NC1=O]

compound AGLU-Glutar-APAc-Hse-Trp-5Clw-Hyp-Glu-leu-[Cys(2Lut)-Apc(DOTA)-Leu-Ile-Cys]-APAc-glu(AGLU)-NH₂ (GIP-049, SEQ ID NO: 52) of the following formula

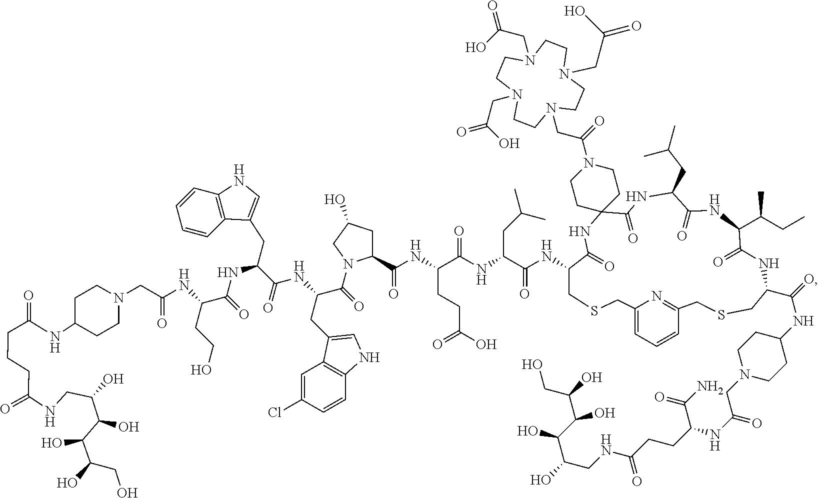 [SMILES: CC[C@H](C)[C@@H]1NC(=O)[C@H](CC(C)C)NC(=O)C2(CCN(C(=O)CN3CCN(CC(=O)O)CCN(CC(=O)O)CCN(CC(=O)O)CC3)CC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CCO)NC(=O)CN2CCC(NC(=O)CCCC(=O)NC[C@H](O)[C@@H](O)[C@H](O)[C@H](O)CO)CC2)CSCc2cccc(n2)CSC[C@@H](C(=O)NC2CCN(CC(=O)N[C@H](CCC(=O)NC[C@H](O)[C@@H](O)[C@H](O)[C@H](O)CO)C(N)=O)CC2)NC1=O]

compound AGLU-Glutar-APAc-Hse-Trp-5Clw-Tap-Glu-leu-[Cys(2Lut)-Apc(DOTA)-Leu-Ile-Cys]-APAc-glu(AGLU)-NH (GIP-051, SEQ ID NO: 53) of the following formula

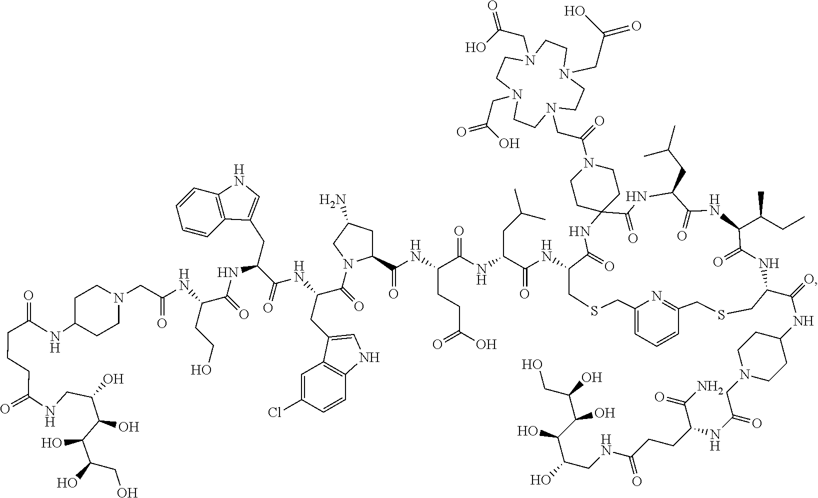 [SMILES: CC[C@H](C)[C@@H]1NC(=O)[C@H](CC(C)C)NC(=O)C2(CCN(C(=O)CN3CCN(CC(=O)O)CCN(CC(=O)O)CCN(CC(=O)O)CC3)CC2)NC(=O)[C@@H](NC(=O)[C@@H](CC(C)C)NC(=O)[C@H](CCC(=O)O)NC(=O)[C@@H]2C[C@@H](N)CN2C(=O)[C@H](Cc2c[nH]c3ccc(Cl)cc23)NC(=O)[C@H](Cc2c[nH]c3ccccc23)NC(=O)[C@H](CCO)NC(=O)CN2CCC(NC(=O)CCCC(=O)NC[C@H](O)[C@@H](O)[C@H](O)[C@H](O)CO)CC2)CSCc2cccc(n2)CSC[C@@H](C(=O)NC2CCN(CC(=O)N[C@H](CCC(=O)NC[C@H](O)[C@@H](O)[C@H](O)[C@H](O)CO)C(N)=O)CC2)NC1=O]

compound AGLU-Glutar-Asp-Trp-5Clw-Tap-Glu-leu-[cys(2Lut)-Apc(DOTA)-Leu-Ile-Cys]-APAc-glu(AGLU)-NH₂ (GIP-051, SEQ ID NO: 54) of the following formula

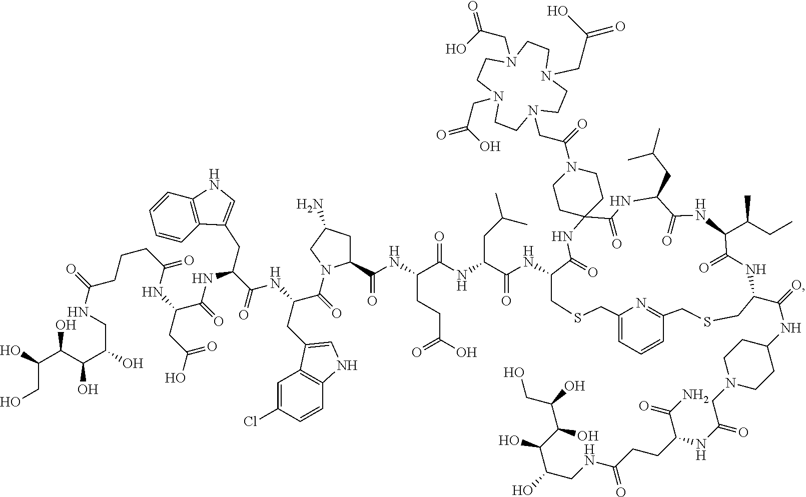 [SMILES: CC[C@H](C)[C@@H]1NC(=O)[C@H](CC(C)C)NC(=O)C2(CCN(C(=O)CN3CCN(CC(=O)O)CCN(CC(=O)O)CCN(CC(=O)O)CC3)CC2)NC(=O)[C@@H](NC(=O)[C@@H](CC(C)C)NC(=O)[C@H](CCC(=O)O)NC(=O)[C@@H]2C[C@@H](N)CN2C(=O)[C@H](Cc2c[nH]c3ccc(Cl)cc23)NC(=O)[C@H](Cc2c[nH]c3ccccc23)NC(=O)[C@H](CC(=O)O)NC(=O)CCCC(=O)NC[C@H](O)[C@@H](O)[C@H](O)[C@H](O)CO)CSCc2cccc(n2)CSC[C@@H](C(=O)NC2CCN(CC(=O)N[C@H](CCC(=O)NC[C@H](O)[C@@H](O)[C@H](O)[C@H](O)CO)C(N)=O)CC2)NC1=O]

compound AGLU-Glutar-Asp-Trp-5Clw-Tap-Glu-leu-[cys(2Lut)-Apc(DOTA)-Leu-Ile-cys]-APAc-glu(AGLU)-NH₂ (GIP-052, SEQ ID NO: 55) of the following formula

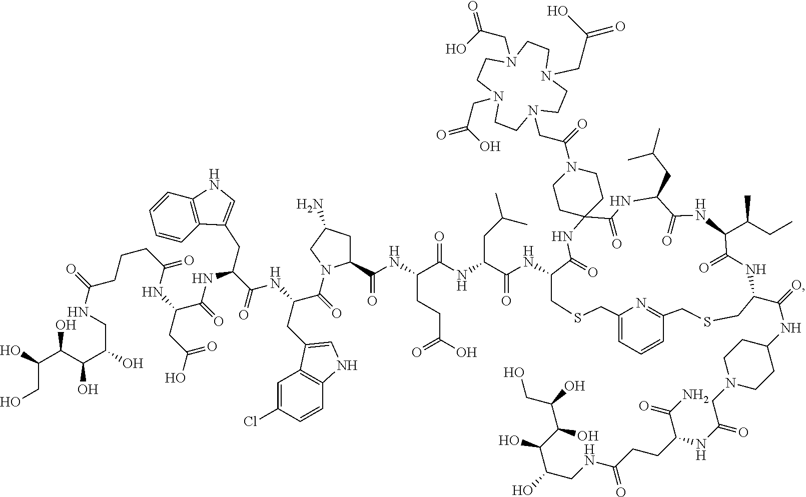 [SMILES: CC[C@H](C)[C@@H]1NC(=O)[C@H](CC(C)C)NC(=O)C2(CCN(C(=O)CN3CCN(CC(=O)O)CCN(CC(=O)O)CCN(CC(=O)O)CC3)CC2)NC(=O)[C@@H](NC(=O)[C@@H](CC(C)C)NC(=O)[C@H](CCC(=O)O)NC(=O)[C@@H]2C[C@@H](N)CN2C(=O)[C@H](Cc2c[nH]c3ccc(Cl)cc23)NC(=O)[C@H](Cc2c[nH]c3ccccc23)NC(=O)[C@H](CC(=O)O)NC(=O)CCCC(=O)NC[C@H](O)[C@@H](O)[C@H](O)[C@H](O)CO)CSCc2cccc(n2)CSC[C@@H](C(=O)NC2CCN(CC(=O)N[C@H](CCC(=O)NC[C@H](O)[C@@H](O)[C@H](O)[C@H](O)CO)C(N)=O)CC2)NC1=O]

compound DOTA-Ttds-Asp-Trp-5Clw-Pro-Glu-leu-[Cys(3MeBn)-Apc(DOTA)-Leu-Ile-AET] (GIP-053, SEQ ID NO: 56) of the following formula

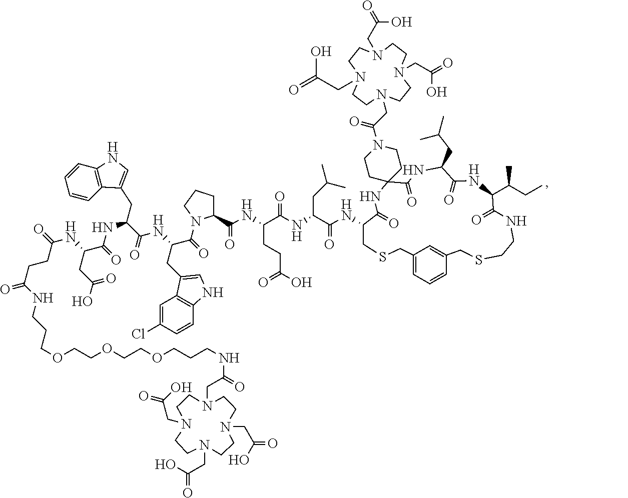 [SMILES: CC[C@H](C)[C@@H]1NC(=O)[C@H](CC(C)C)NC(=O)C2(CCN(C(=O)CN3CCN(CC(=O)O)CCN(CC(=O)O)CCN(CC(=O)O)CC3)CC2)NC(=O)[C@@H](NC(=O)[C@@H](CC(C)C)NC(=O)[C@H](CCC(=O)O)NC(=O)[C@@H]2CCCN2C(=O)[C@H](Cc2c[nH]c3ccc(Cl)cc23)NC(=O)[C@H](Cc2c[nH]c3ccccc23)NC(=O)[C@H](CC(=O)O)NC(=O)CCC(=O)NCCCOCCOCCOCCCNC(=O)CN2CCN(CC(=O)O)CCN(CC(=O)O)CCN(CC(=O)O)CC2)CSCc2cccc(c2)CSCCNC1=O]

compound DOTA-Ttds-Asp-Trp-5Clw-Hyp-Glu-leu-[Cys(2Lut)-Thp-Leu-Ile-Cys]-Lys(DOTA)-NH₂ (GIP-054, SEQ ID NO: 57) of the following formula

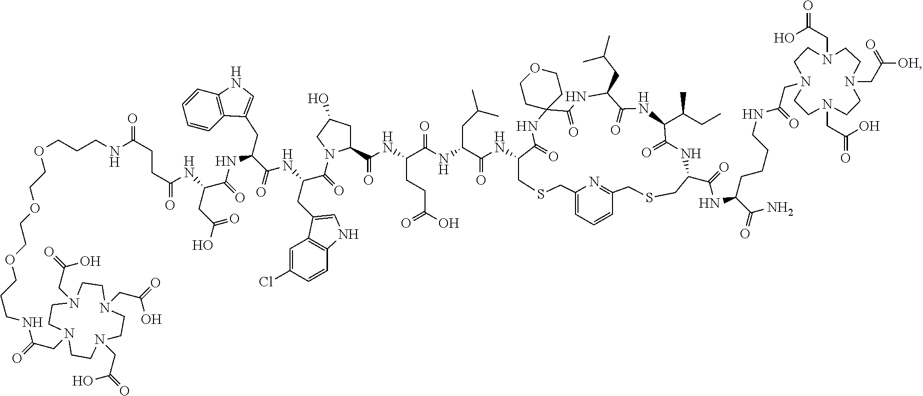 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CC(=O)O)NC(=O)CCC(=O)NCCCOCCOCCOCCCNC(=O)CN2CCN(CC(=O)O)CCN(CC(=O)O)CCN(CC(=O)O)CC2)CSCc2cccc(n2)CSC[C@@H](C(=O)N[C@@H](CCCCNC(=O)CN2CCN(CC(=O)O)CCN(CC(=O)O)CCN(CC(=O)O)CC2)C(N)=O)NC1=O]

compound DOTA-Ttds-Asp-Trp-5Clw-Hyp-Glu-leu-[Cys(2Lut)-Apc(DOTA)-Leu-Ile-Cys]-Lys(DOTA)-NH₂ (GIP-055, SEQ ID NO: 58) of the following formula

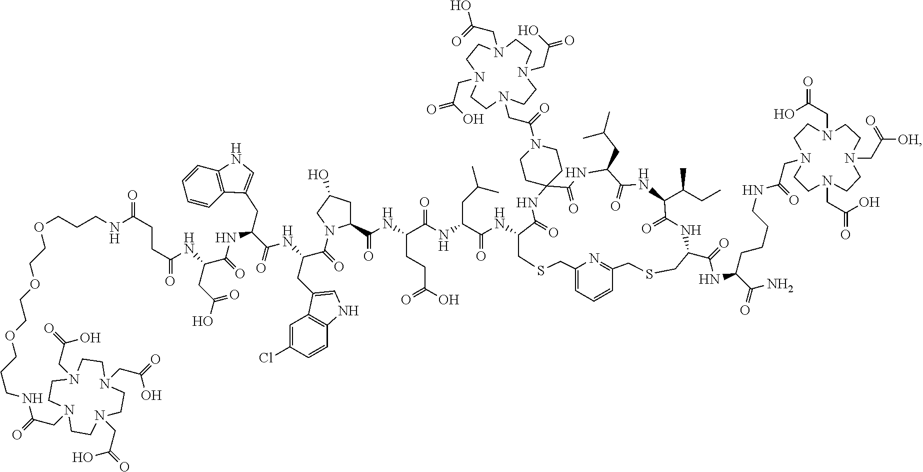 [SMILES: CC[C@H](C)[C@@H]1NC(=O)[C@H](CC(C)C)NC(=O)C2(CCN(C(=O)CN3CCN(CC(=O)O)CCN(CC(=O)O)CCN(CC(=O)O)CC3)CC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CC(=O)O)NC(=O)CCC(=O)NCCCOCCOCCOCCCNC(=O)CN2CCN(CC(=O)O)CCN(CC(=O)O)CCN(CC(=O)O)CC2)CSCc2cccc(n2)CSC[C@@H](C(=O)N[C@@H](CCCCNC(=O)CN2CCN(CC(=O)O)CCN(CC(=O)O)CCN(CC(=O)O)CC2)C(N)=O)NC1=O]

compound DOTA-Ttds-Asp-Trp-5Clw-Tap(DOTA)-Glu-leu-[Cys(2Lut)-Apc(DOTA)-Leu-Ile-Cys]-Lys(DOTA)-NH₂ (GIP-056, SEQ ID NO: 59) of the following formula

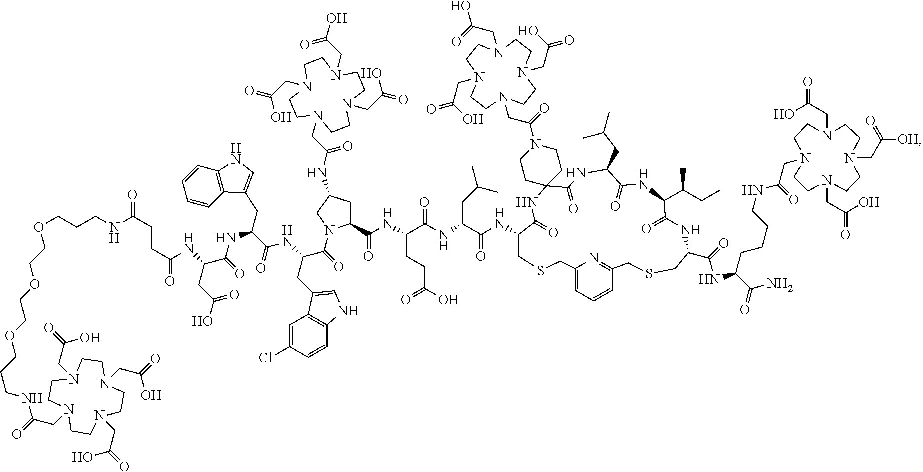 [SMILES: CC[C@H](C)[C@@H]1NC(=O)[C@H](CC(C)C)NC(=O)C2(CCN(C(=O)CN3CCN(CC(=O)O)CCN(CC(=O)O)CCN(CC(=O)O)CC3)CC2)NC(=O)[C@@H](NC(=O)[C@@H](CC(C)C)NC(=O)[C@H](CCC(=O)O)NC(=O)[C@@H]2C[C@@H](NC(=O)CN3CCN(CC(=O)O)CCN(CC(=O)O)CCN(CC(=O)O)CC3)CN2C(=O)[C@H](Cc2c[nH]c3ccc(Cl)cc23)NC(=O)[C@H](Cc2c[nH]c3ccccc23)NC(=O)[C@H](CC(=O)O)NC(=O)CCC(=O)NCCCOCCOCCOCCCNC(=O)CN2CCN(CC(=O)O)CCN(CC(=O)O)CCN(CC(=O)O)CC2)CSCc2cccc(n2)CSC[C@@H](C(=O)N[C@@H](CCCCNC(=O)CN2CCN(CC(=O)O)CCN(CC(=O)O)CCN(CC(=O)O)CC2)C(N)=O)NC1=O]

compound AGLU-Glutar-APAc-Asp-Trp-5Clw-Tap-Glu-leu-[Cys(2Lut)-Apc(GaDOTA-glu(AGLU))-Leu-Ile-Cys]-APAc-glu(AGLU)-NH₂ (GIP-059, SEQ ID NO: 60) of the following formula

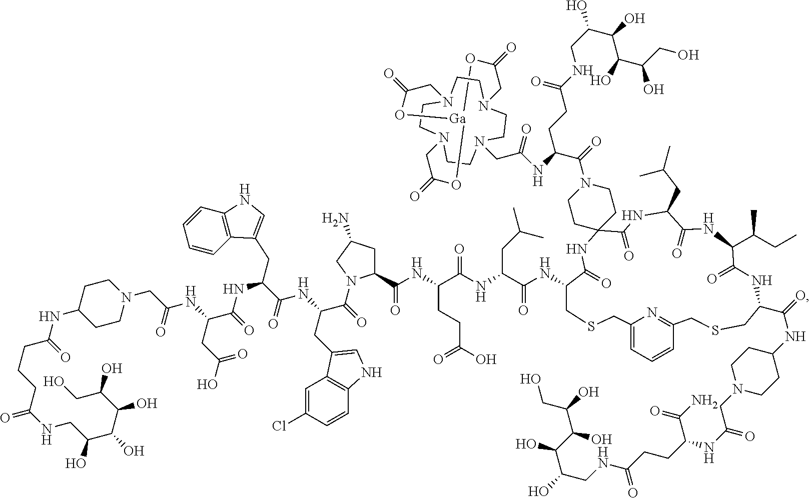

compound AGLU-Glutar-APAc-Asp-Trp-5Clw-Tap-Glu-leu-[Cys(2Lut)-Apc(InDOTA-glu(AGLU))-Leu-Ile-Cys]-APAc-glu(AGLU)-NH₂ (GIP-060, SEQ ID NO: 61) of the following formula

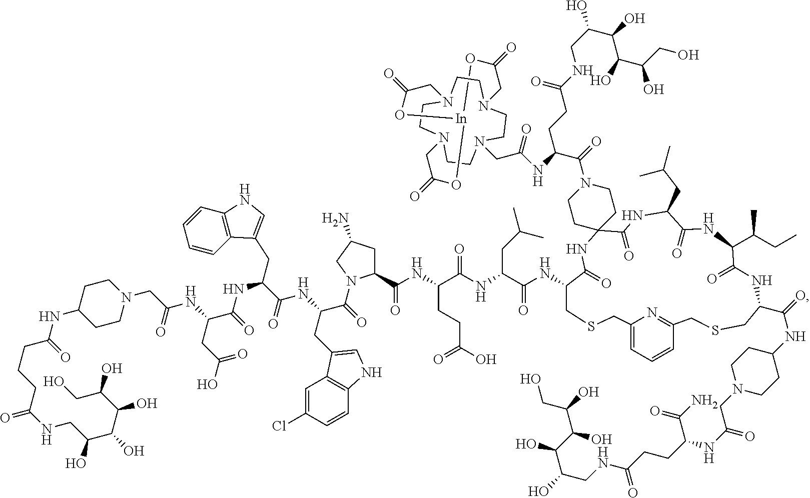

compound AGLU-Glutar-APAc-Asp-Trp-5Clw-Tap-Glu-leu-[Cys(2Lut)-Apc(LuDOTA-glu(AGLU))-Leu-Ile-Cys]-APAc-glu(AGLU)-NH₂ (GIP-061, SEQ ID NO: 62) of the following formula

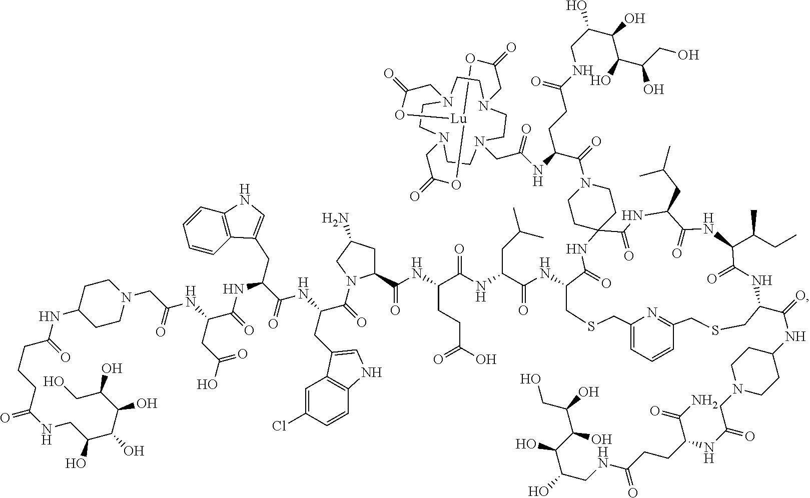

compound LuDOTA-glu(AGLU)-APAc-Asp-Trp-5Clw-Hyp-Glu-leu-[Cys(2Lut)-Thp-Leu-Ile-Cys]-APAc-glu(AGLU)-NH₂ (GIP-062, SEQ ID NO: 63) of the following formula

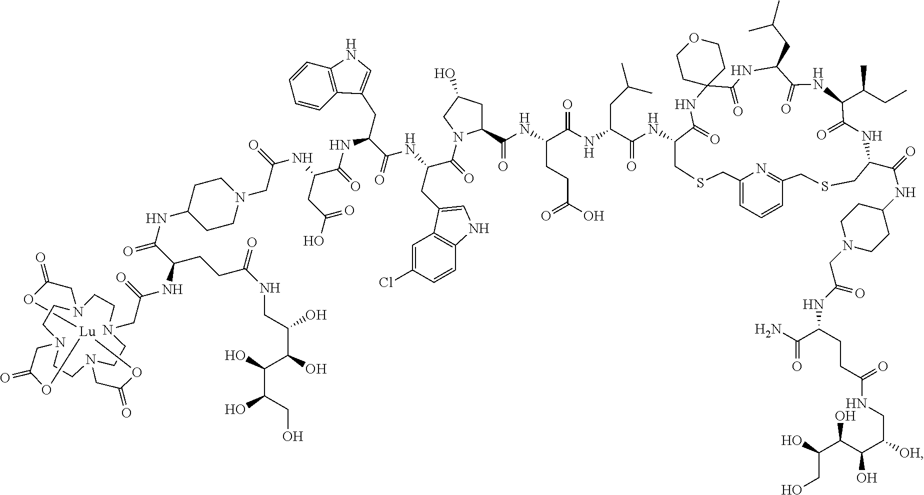

compound InDOTA-glu(AGLU)-APAc-Asp-Trp-5Clw-Hyp-Glu-leu-[Cys(2Lut)-Thp-Leu-Ile-Cys]-APAc-glu(AGLU)-NH₂ (GIP-063, SEQ ID NO: 64) of the following formula

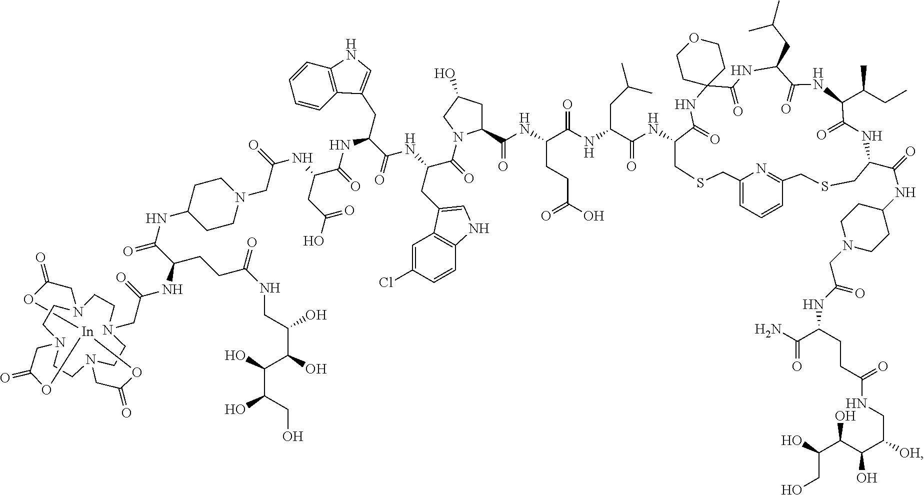

compound EuDOTA-glu(AGLU)-APAc-Asp-Trp-5Clw-Hyp-Glu-leu-[Cys(2Lut)-Thp-Leu-Ile-Cys]-APAc-glu(AGLU)-NH₂ (GIP-064, SEQ ID NO: 65) of the following formula

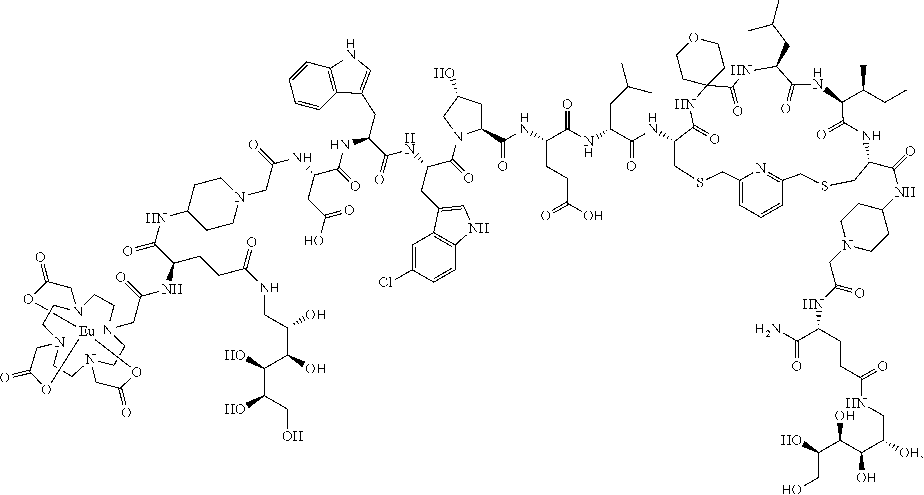

compound AGLU-Glutar-Asp-Trp-5Clw-Hyp-Glu-leu-[Cys(2Lut)-Apc(InDOTA)-Leu-Ile-Cys]-APAc-glu(AGLU)-NH₂ (GIP-065, SEQ ID NO: 66) of the following formula

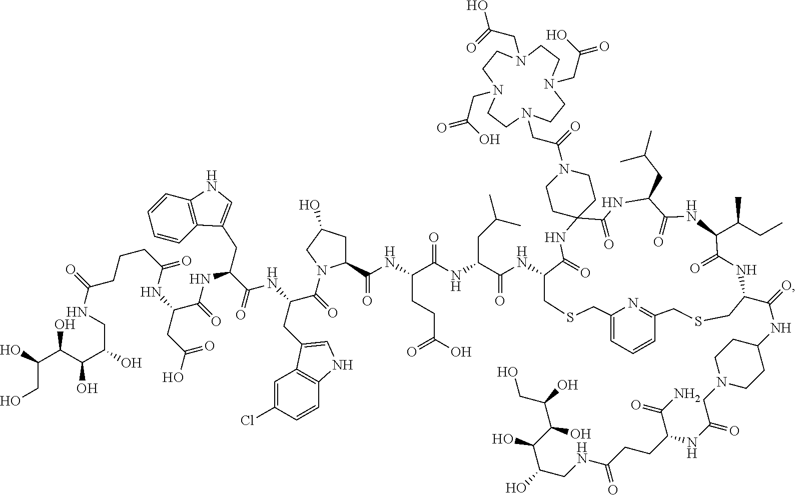 [SMILES: CC[C@H](C)[C@@H]1NC(=O)[C@H](CC(C)C)NC(=O)C2(CCN(C(=O)CN3CCN(CC(=O)O)CCN(CC(=O)O)CCN(CC(=O)O)CC3)CC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CC(=O)O)NC(=O)CCCC(=O)NC[C@H](O)[C@@H](O)[C@H](O)[C@H](O)CO)CSCc2cccc(n2)CSC[C@@H](C(=O)NC2CCN(CC(=O)N[C@H](CCC(=O)NC[C@H](O)[C@@H](O)[C@H](O)[C@H](O)CO)C(N)=O)CC2)NC1=O]

compound AGLU-Glutar-Asp-Trp-5Clw-Hyp-Glu-leu-[Cys(2Lut)-Apc(LuDOTA)-Leu-Ile-Cys]-APAc-glu(AGLU)-NH₂ (GIP-066, SEQ ID NO: 67) of the following formula

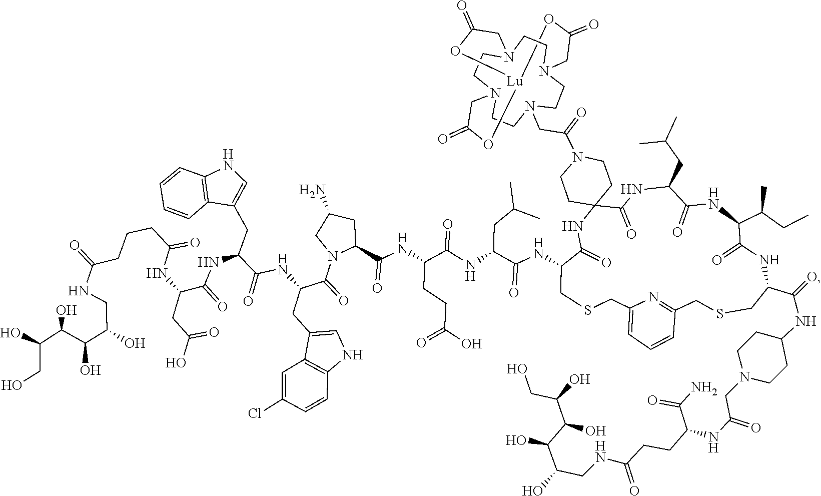

compound AGLU-Glutar-Asp-Trp-5Clw-Tap-Glu-leu-[Cys(2Lut)-Apc(LuDOTA)-Leu-Ile-Cys]-APAc-glu(AGLU)-NH₂ (GIP-067, SEQ ID NO: 68) of the following formula

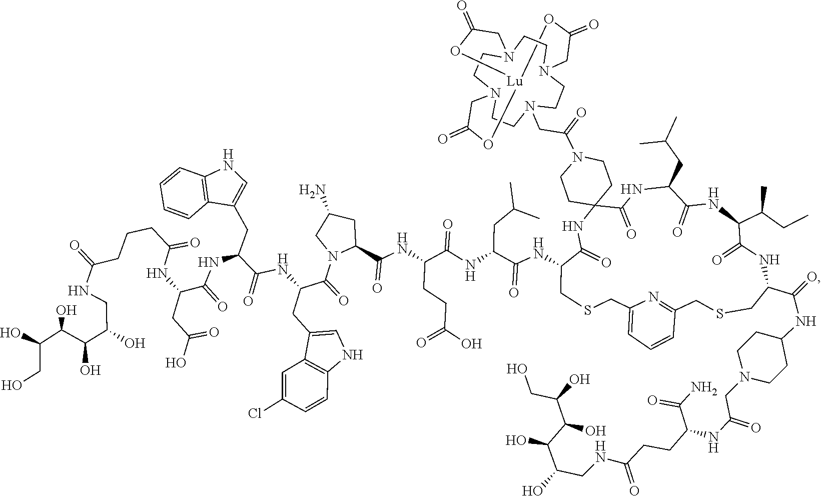

compound AGLU-Glutar-Asp-Trp-5Clw-Tap-Glu-leu-[Cys(2Lut)-Apc (InDOTA)-Leu-Ile-Cys]-APAc-glu (AGLU)-NH₂ (GIP-068, SEQ ID NO: 69) of the following formula

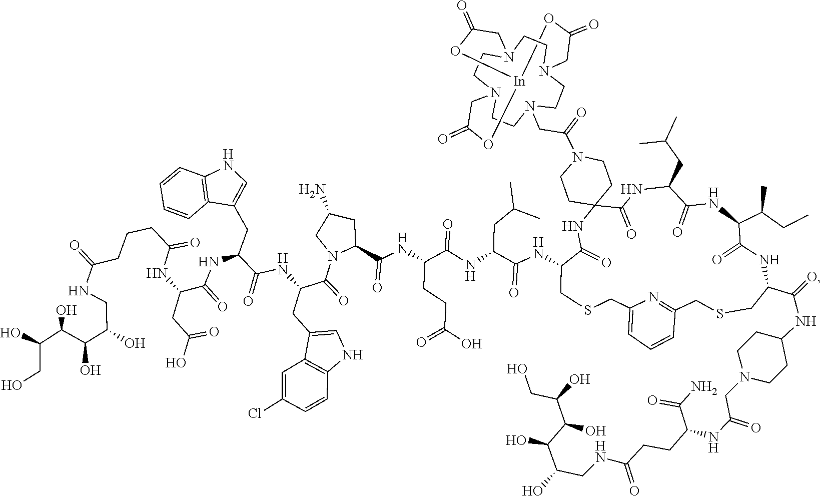

compound AGLU-Glutar-Asp-Trp-5Clw-Tap-Glu-leu-[Cys (2Lut)-Apc(GaDOTA)-Leu-Ile-Cys]-APAc-glu(AGLU)-NH₂ (GIP-069, SEQ ID NO: 70) of the following formula

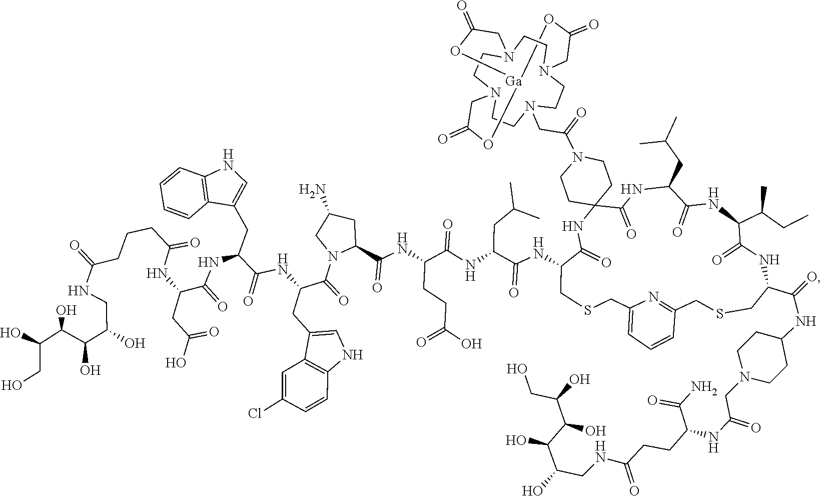

compound AGLU-Glutar-Asp-Trp-5Clw-Hyp-Glu-leu-[Cys(2Lut)-Apc(GaDOTA)-Leu-Ile-Cys]-APAc-glu(AGLU)-NH₂ (GIP-070, SEQ ID NO: 71) of the following formula

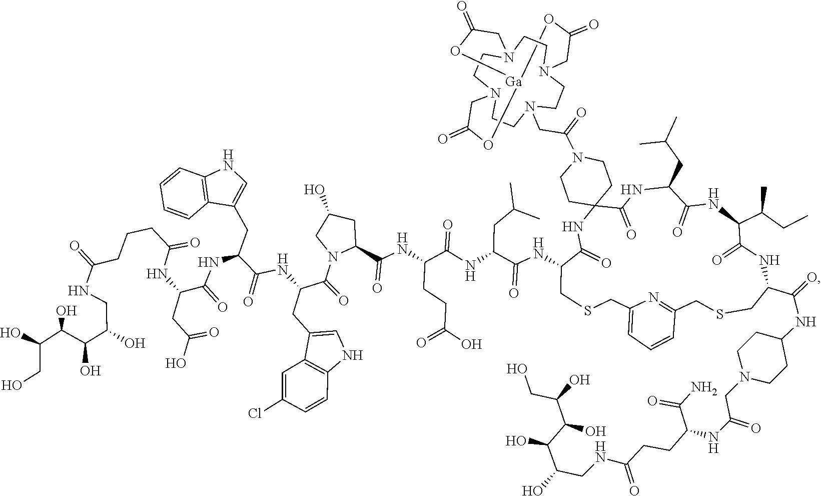

compound AGLU-Glutar-Asp-Trp-5Clw-Tap-Glu-leu-[Cys(2Lut)-Apc(GaNODAGA)-Leu-Ile-Cys]-APAc-glu(AGLU)-NH₂ (GIP-071, SEQ ID NO: 72) of the following formula

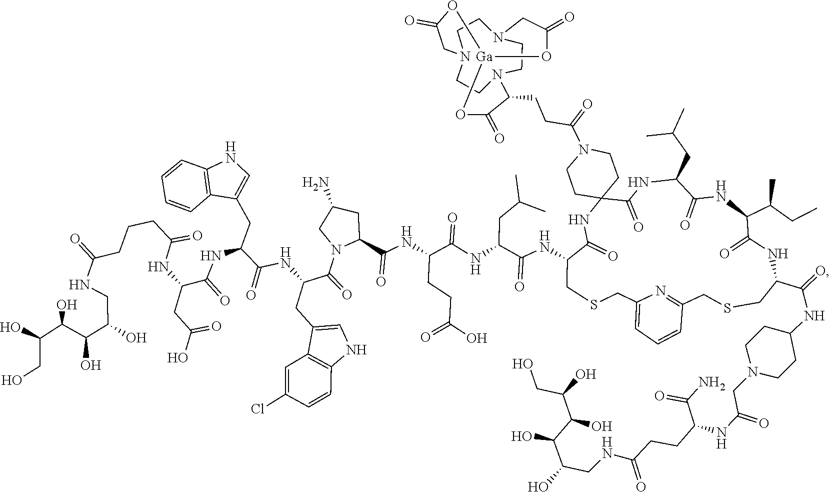

compound AGLU-Glutar-Asp-Trp-5Clw-Hyp-Glu-leu-[Cys(2Lut)-Apc(DOTA)-Leu-Ile-AET] (GIP-072, SEQ ID NO: 73) of the following formula

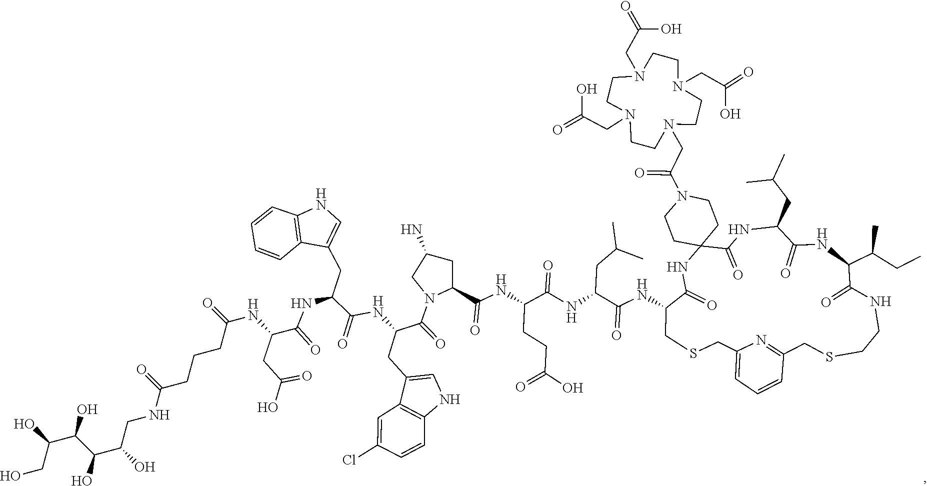 [SMILES: CC[C@H](C)[C@@H]1NC(=O)[C@H](CC(C)C)NC(=O)C2(CCN(C(=O)CN3CCN(CC(=O)O)CCN(CC(=O)O)CCN(CC(=O)O)CC3)CC2)NC(=O)[C@@H](NC(=O)[C@@H](CC(C)C)NC(=O)[C@H](CCC(=O)O)NC(=O)[C@@H]2C[C@@H](N)CN2C(=O)[C@H](Cc2c[nH]c3ccc(Cl)cc23)NC(=O)[C@H](Cc2c[nH]c3ccccc23)NC(=O)[C@H](CC(=O)O)NC(=O)CCCC(=O)NC[C@H](O)[C@@H](O)[C@H](O)[C@H](O)CO)CSCc2cccc(n2)CSCCNC1=O]

compound HO-Succinyl-Trp-5Clw-Hyp-Glu-leu-[Cys(2Lut)-Apc(AGLU-Glutar)-Leu-Ile-Cys]-APAc-Lys(DOTA)-NH₂ (GIP-073, SEQ ID NO: 74) of the following formula

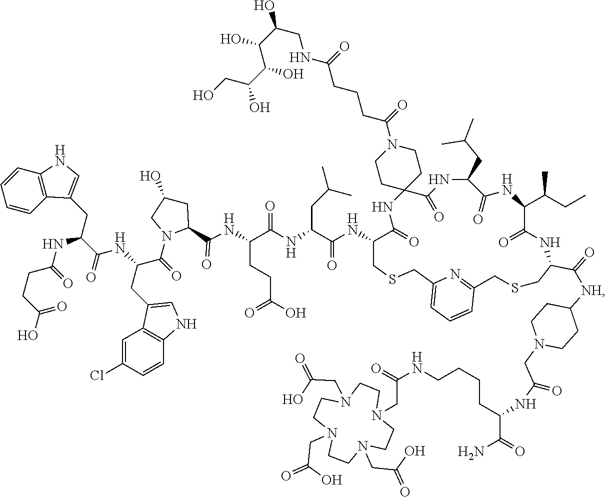 [SMILES: CC[C@H](C)[C@@H]1NC(=O)[C@H](CC(C)C)NC(=O)C2(CCN(C(=O)CCCC(=O)NC[C@H](O)[C@@H](O)[C@H](O)[C@H](O)CO)CC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)CCC(=O)O)CSCc2cccc(n2)CSC[C@@H](C(=O)NC2CCN(CC(=O)N[C@@H](CCCCNC(=O)CN3CCN(CC(=O)O)CCN(CC(=O)O)CCN(CC(=O)O)CC3)C(N)=O)CC2)NC1=O]

compound Ac-Asp-Trp-1Ni-Pro-Glu-Leu-[Cys(3MeBn)-Aib-Leu-Ile-Cys]-Ttds-Lys(DOTA)-NH₂ (GIP-074, SEQ ID NO: 75) of the following formula

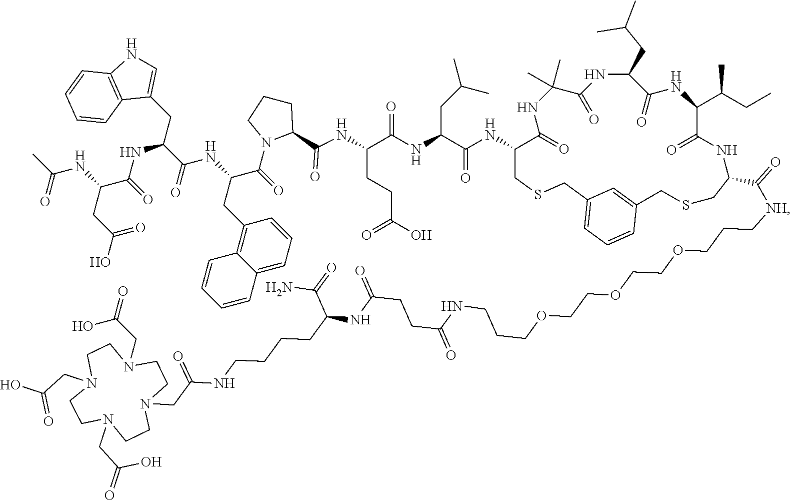 [SMILES: CC[C@H](C)[C@@H]1NC(=O)[C@H](CC(C)C)NC(=O)C(C)(C)NC(=O)[C@@H](NC(=O)[C@H](CC(C)C)NC(=O)[C@H](CCC(=O)O)NC(=O)[C@@H]2CCCN2C(=O)[C@H](Cc2cccc3ccccc23)NC(=O)[C@H](Cc2c[nH]c3ccccc23)NC(=O)[C@H](CC(=O)O)NC(C)=O)CSCc2cccc(c2)CSC[C@@H](C(=O)NCCCOCCOCCOCCCNC(=O)CCC(=O)N[C@@H](CCCCNC(=O)CN2CCN(CC(=O)O)CCN(CC(=O)O)CCN(CC(=O)O)CC2)C(N)=O)NC1=O]

compound Ac-Asp-Trp-Bta-Pro-Glu-Leu-[Cys(3MeBn)-Aib-Leu-Ile-Cys]-Ttds-Lys(DOTA)-NH₂ (GIP-075, SEQ ID NO: 76) of the following formula

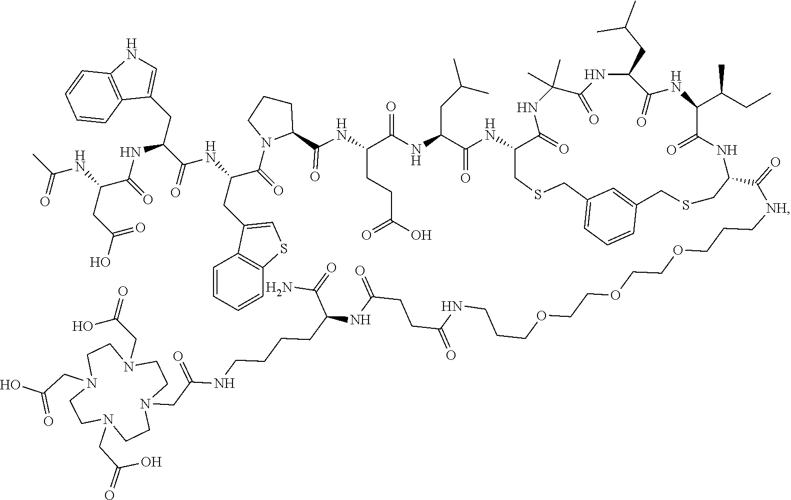 [SMILES: CC[C@H](C)[C@@H]1NC(=O)[C@H](CC(C)C)NC(=O)C(C)(C)NC(=O)[C@@H](NC(=O)[C@H](CC(C)C)NC(=O)[C@H](CCC(=O)O)NC(=O)[C@@H]2CCCN2C(=O)[C@H](Cc2csc3ccccc23)NC(=O)[C@H](Cc2c[nH]c3ccccc23)NC(=O)[C@H](CC(=O)O)NC(C)=O)CSCc2cccc(c2)CSC[C@@H](C(=O)NCCCOCCOCCOCCCNC(=O)CCC(=O)N[C@@H](CCCCNC(=O)CN2CCN(CC(=O)O)CCN(CC(=O)O)CCN(CC(=O)O)CC2)C(N)=O)NC1=O]

compound Ac-Asp-Trp-5Clw-Pro-Glu-Leu-[Cys(3MeBn)-Aib-Leu-Ile-Cys]-Ttds-Lys(DOTA)-NH₂ (GIP-076, SEQ ID NO: 77) of the following formula

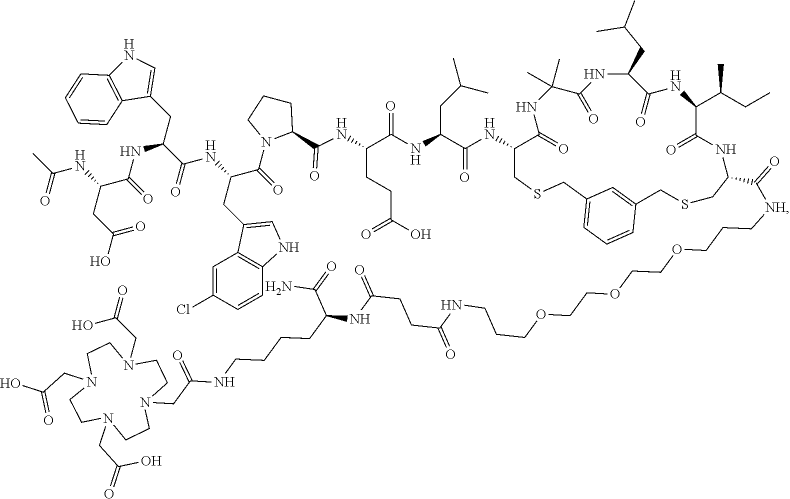 [SMILES: CC[C@H](C)[C@@H]1NC(=O)[C@H](CC(C)C)NC(=O)C(C)(C)NC(=O)[C@@H](NC(=O)[C@H](CC(C)C)NC(=O)[C@H](CCC(=O)O)NC(=O)[C@@H]2CCCN2C(=O)[C@H](Cc2c[nH]c3ccc(Cl)cc23)NC(=O)[C@H](Cc2c[nH]c3ccccc23)NC(=O)[C@H](CC(=O)O)NC(C)=O)CSCc2cccc(c2)CSC[C@@H](C(=O)NCCCOCCOCCOCCCNC(=O)CCC(=O)N[C@@H](CCCCNC(=O)CN2CCN(CC(=O)O)CCN(CC(=O)O)CCN(CC(=O)O)CC2)C(N)=O)NC1=O]

Compound DOTA-Ttds-Asp-Trp-5Clw-Pro-Glu-leu-[Cys(3MeBn)-Aib-Leu-Ile-AET] (GIP-077, SEQ ID NO: 78) of the following formula

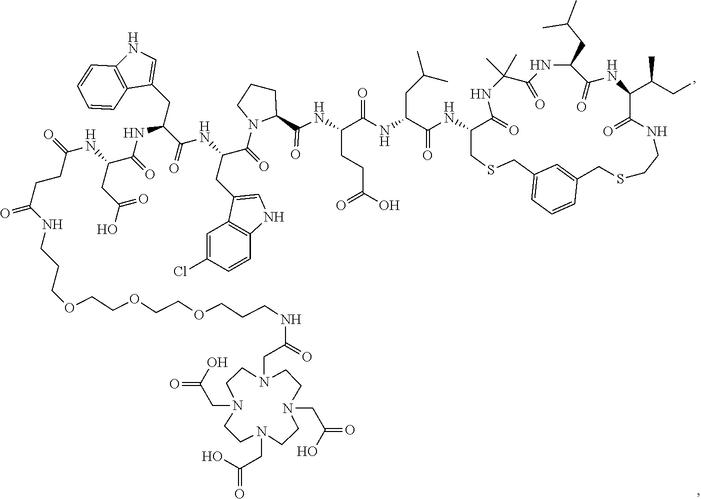

compound Ac-Asp-Trp-1Ni-Pro-Glu-leu-[Cys(3MeBn)-Aib-Leu-Ile-Cys]-Ttds-Lys(DOTA)-NH₂ (GIP-078, SEQ ID NO: 79) of the following formula

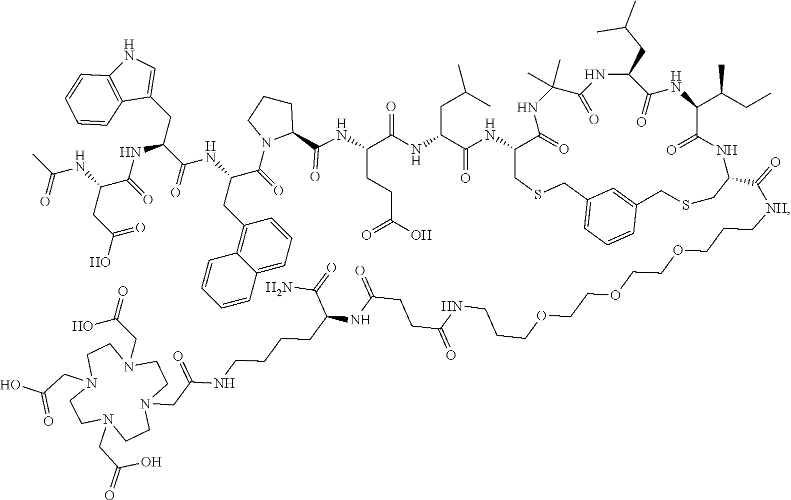 [SMILES: CC[C@H](C)[C@@H]1NC(=O)[C@H](CC(C)C)NC(=O)C(C)(C)NC(=O)[C@@H](NC(=O)[C@@H](CC(C)C)NC(=O)[C@H](CCC(=O)O)NC(=O)[C@@H]2CCCN2C(=O)[C@H](Cc2cccc3ccccc23)NC(=O)[C@H](Cc2c[nH]c3ccccc23)NC(=O)[C@H](CC(=O)O)NC(C)=O)CSCc2cccc(c2)CSC[C@@H](C(=O)NCCCOCCOCCOCCCNC(=O)CCC(=O)N[C@@H](CCCCNC(=O)CN2CCN(CC(=O)O)CCN(CC(=O)O)CCN(CC(=O)O)CC2)C(N)=O)NC1=O]

compound Ac-Asp-Trp-5Clw-Pro-Glu-leu-[Cys(3MeBn)-Aib-Leu-Ile-Cys]-Ttds-Lys(DOTA)-NH₂ (GIP-079, SEQ ID NO: 80) of the following formula

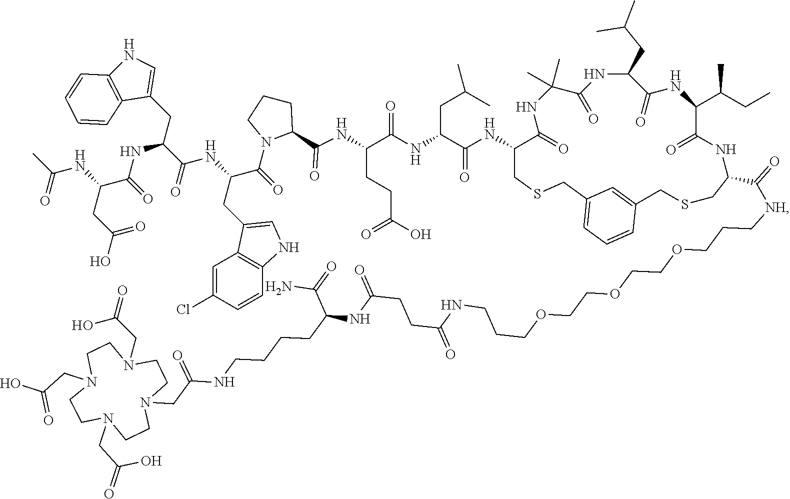 [SMILES: CC[C@H](C)[C@@H]1NC(=O)[C@H](CC(C)C)NC(=O)C(C)(C)NC(=O)[C@@H](NC(=O)[C@@H](CC(C)C)NC(=O)[C@H](CCC(=O)O)NC(=O)[C@@H]2CCCN2C(=O)[C@H](Cc2c[nH]c3ccc(Cl)cc23)NC(=O)[C@H](Cc2c[nH]c3ccccc23)NC(=O)[C@H](CC(=O)O)NC(C)=O)CSCc2cccc(c2)CSC[C@@H](C(=O)NCCCOCCOCCOCCCNC(=O)CCC(=O)N[C@@H](CCCCNC(=O)CN2CCN(CC(=O)O)CCN(CC(=O)O)CCN(CC(=O)O)CC2)C(N)=O)NC1=O]

compound Ac-Asp-Trp-1Ni-Pro-Glu-leu-[Cys(3MeBn)-Egz-Leu-Ile-Cys]-Ttds-Lys(DOTA)-NH₂ (GIP-080, SEQ ID NO: 81) of the following formula

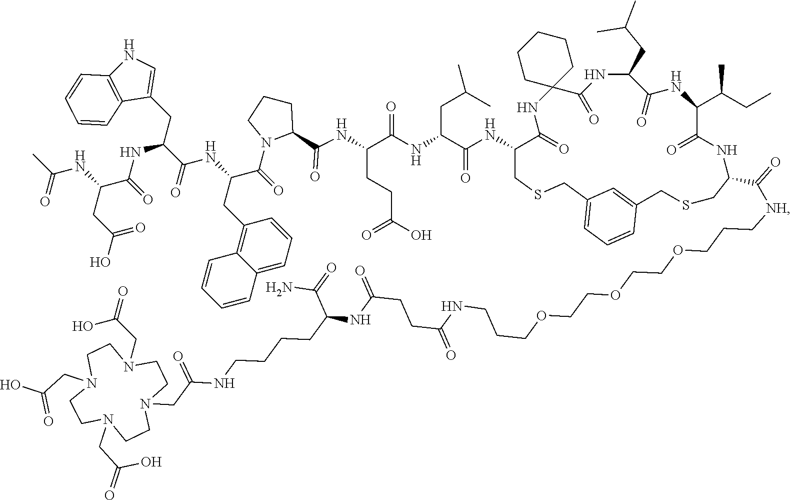 [SMILES: CC[C@H](C)[C@@H]1NC(=O)[C@H](CC(C)C)NC(=O)C2(CCCCC2)NC(=O)[C@@H](NC(=O)[C@@H](CC(C)C)NC(=O)[C@H](CCC(=O)O)NC(=O)[C@@H]2CCCN2C(=O)[C@H](Cc2cccc3ccccc23)NC(=O)[C@H](Cc2c[nH]c3ccccc23)NC(=O)[C@H](CC(=O)O)NC(C)=O)CSCc2cccc(c2)CSC[C@@H](C(=O)NCCCOCCOCCOCCCNC(=O)CCC(=O)N[C@@H](CCCCNC(=O)CN2CCN(CC(=O)O)CCN(CC(=O)O)CCN(CC(=O)O)CC2)C(N)=O)NC1=O]

compound Ac-Asp-Trp-5Fw-Pro-Glu-leu-[Cys(3MeBn)-Aib-Leu-Ile-Cys]-Ttds-Lys(DOTA)-NH₂ (GIP-081, SEQ ID NO: 82) of the following formula

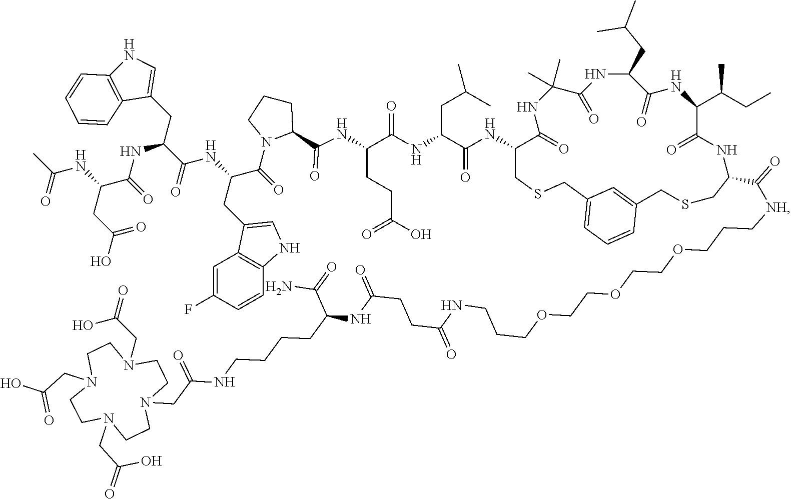 [SMILES: CC[C@H](C)[C@@H]1NC(=O)[C@H](CC(C)C)NC(=O)C(C)(C)NC(=O)[C@@H](NC(=O)[C@@H](CC(C)C)NC(=O)[C@H](CCC(=O)O)NC(=O)[C@@H]2CCCN2C(=O)[C@H](Cc2c[nH]c3ccc(F)cc23)NC(=O)[C@H](Cc2c[nH]c3ccccc23)NC(=O)[C@H](CC(=O)O)NC(C)=O)CSCc2cccc(c2)CSC[C@@H](C(=O)NCCCOCCOCCOCCCNC(=O)CCC(=O)N[C@@H](CCCCNC(=O)CN2CCN(CC(=O)O)CCN(CC(=O)O)CCN(CC(=O)O)CC2)C(N)=O)NC1=O]

compound Ac-Asp-Trp-5Brw-Pro-Glu-leu-[Cys(3MeBn)-Aib-Leu-Ile-Cys]-Ttds-Lys(DOTA)-NH₂ (GIP-082, SEQ ID NO: 83) of the following formula

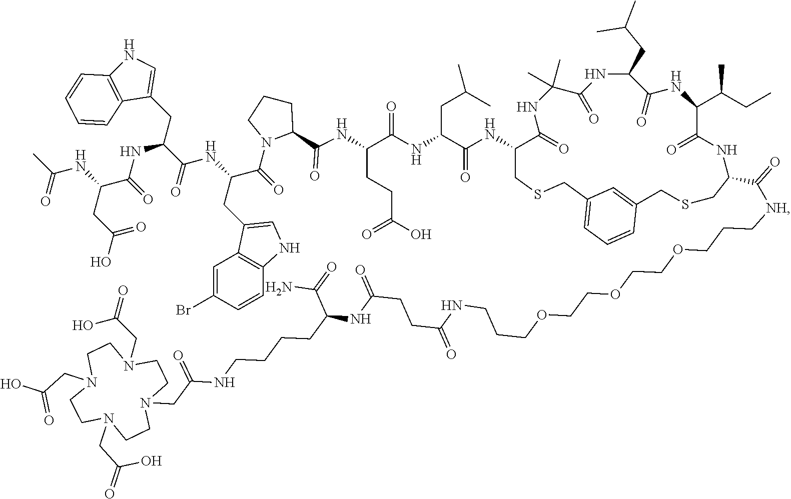 [SMILES: CC[C@H](C)[C@@H]1NC(=O)[C@H](CC(C)C)NC(=O)C(C)(C)NC(=O)[C@@H](NC(=O)[C@@H](CC(C)C)NC(=O)[C@H](CCC(=O)O)NC(=O)[C@@H]2CCCN2C(=O)[C@H](Cc2c[nH]c3ccc(Br)cc23)NC(=O)[C@H](Cc2c[nH]c3ccccc23)NC(=O)[C@H](CC(=O)O)NC(C)=O)CSCc2cccc(c2)CSC[C@@H](C(=O)NCCCOCCOCCOCCCNC(=O)CCC(=O)N[C@@H](CCCCNC(=O)CN2CCN(CC(=O)O)CCN(CC(=O)O)CCN(CC(=O)O)CC2)C(N)=O)NC1=O]

compound Ac-Asp-5Fw-1Ni-Pro-Glu-leu-[Cys(3MeBn)-Aib-Leu-Ile-Cys]-Ttds-Lys(DOTA)-NH₂ (GIP-083, SEQ ID NO: 84) of the following formula

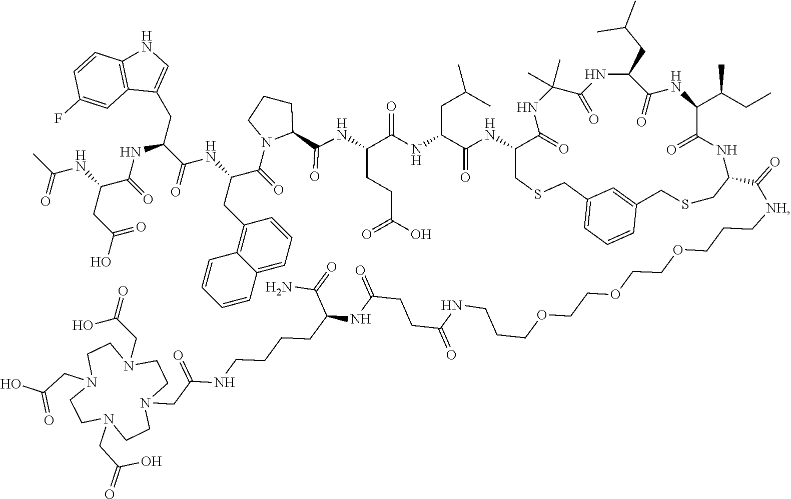 [SMILES: CC[C@H](C)[C@@H]1NC(=O)[C@H](CC(C)C)NC(=O)C(C)(C)NC(=O)[C@@H](NC(=O)[C@@H](CC(C)C)NC(=O)[C@H](CCC(=O)O)NC(=O)[C@@H]2CCCN2C(=O)[C@H](Cc2cccc3ccccc23)NC(=O)[C@H](Cc2c[nH]c3ccc(F)cc23)NC(=O)[C@H](CC(=O)O)NC(C)=O)CSCc2cccc(c2)CSC[C@@H](C(=O)NCCCOCCOCCOCCCNC(=O)CCC(=O)N[C@@H](CCCCNC(=O)CN2CCN(CC(=O)O)CCN(CC(=O)O)CCN(CC(=O)O)CC2)C(N)=O)NC1=O]

compound Ac-Asp-6Fw-Ni-Pro-Glu-leu-[Cys(3MeBn)-Aib-Leu-Ile-Cys]-Ttds-Lys(DOTA)-NH₂ (GIP-084, SEQ ID NO: 85) of the following formula

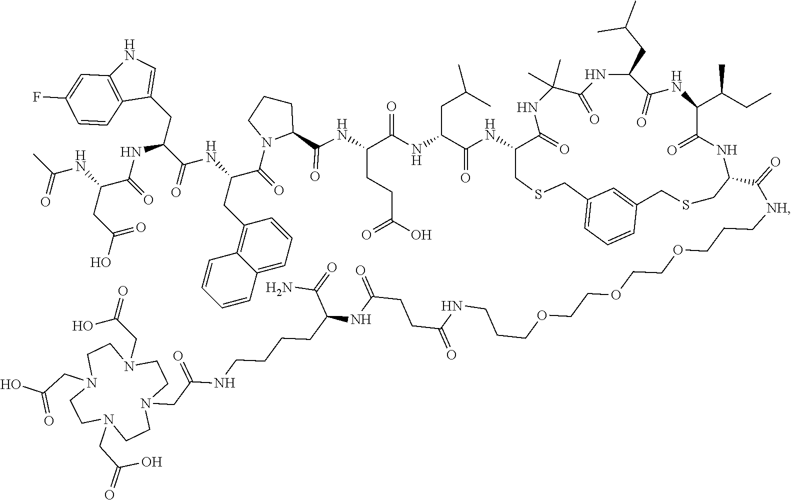 [SMILES: CC[C@H](C)[C@@H]1NC(=O)[C@H](CC(C)C)NC(=O)C(C)(C)NC(=O)[C@@H](NC(=O)[C@@H](CC(C)C)NC(=O)[C@H](CCC(=O)O)NC(=O)[C@@H]2CCCN2C(=O)[C@H](Cc2cccc3ccccc23)NC(=O)[C@H](Cc2c[nH]c3cc(F)ccc23)NC(=O)[C@H](CC(=O)O)NC(C)=O)CSCc2cccc(c2)CSC[C@@H](C(=O)NCCCOCCOCCOCCCNC(=O)CCC(=O)N[C@@H](CCCCNC(=O)CN2CCN(CC(=O)O)CCN(CC(=O)O)CCN(CC(=O)O)CC2)C(N)=O)NC1=O]

compound HO-Succinyl-Trp-1Ni-Pro-Glu-Leu-[Cys(3MeBn)-Aib-Leu-Ile-Cys]-Ttds-Lys(DOTA)-NH₂ (GIP-085, SEQ ID NO: 86) of the following formula

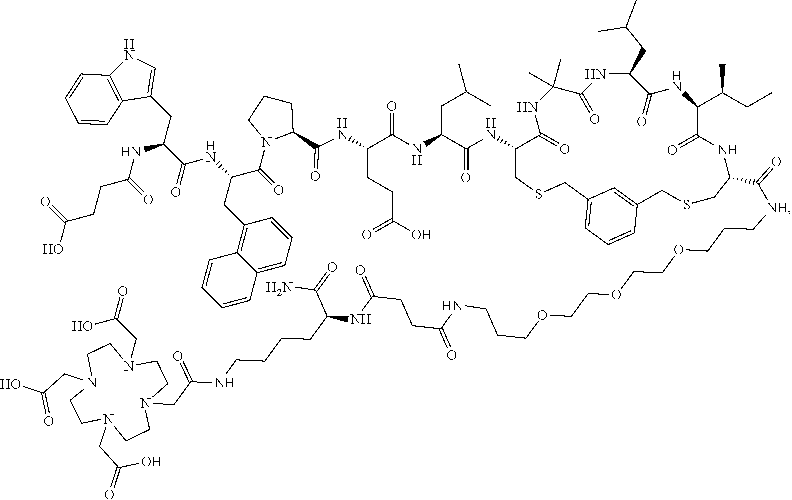 [SMILES: CC[C@H](C)[C@@H]1NC(=O)[C@H](CC(C)C)NC(=O)C(C)(C)NC(=O)[C@@H](NC(=O)[C@H](CC(C)C)NC(=O)[C@H](CCC(=O)O)NC(=O)[C@@H]2CCCN2C(=O)[C@H](Cc2cccc3ccccc23)NC(=O)[C@H](Cc2c[nH]c3ccccc23)NC(=O)CCC(=O)O)CSCc2cccc(c2)CSC[C@@H](C(=O)NCCCOCCOCCOCCCNC(=O)CCC(=O)N[C@@H](CCCCNC(=O)CN2CCN(CC(=O)O)CCN(CC(=O)O)CCN(CC(=O)O)CC2)C(N)=O)NC1=O]

compound DOTA-Ttds-Asp-Trp-5Clw-Pro-Glu-leu-[Cys(3MeBn)-Egz-Leu-Ile-AET] (GIP-086, SEQ ID NO: 87) of the following formula

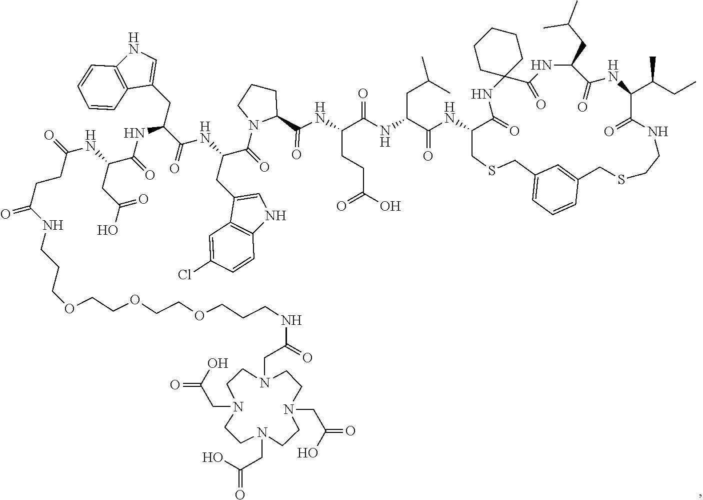 [SMILES: CC[C@H](C)[C@@H]1NC(=O)[C@H](CC(C)C)NC(=O)C2(CCCCC2)NC(=O)[C@@H](NC(=O)[C@@H](CC(C)C)NC(=O)[C@H](CCC(=O)O)NC(=O)[C@@H]2CCCN2C(=O)[C@H](Cc2c[nH]c3ccc(Cl)cc23)NC(=O)[C@H](Cc2c[nH]c3ccccc23)NC(=O)[C@H](CC(=O)O)NC(=O)CCC(=O)NCCCOCCOCCOCCCNC(=O)CN2CCN(CC(=O)O)CCN(CC(=O)O)CCN(CC(=O)O)CC2)CSCc2cccc(c2)CSCCNC1=O]

compound DOTA-Ttds-Asp-Trp-5Clw-Pro-Glu-leu-[Cys(3MeBn)-Thp-Leu-Ile-AET] (GIP-087, SEQ ID NO: 88) of the following formula

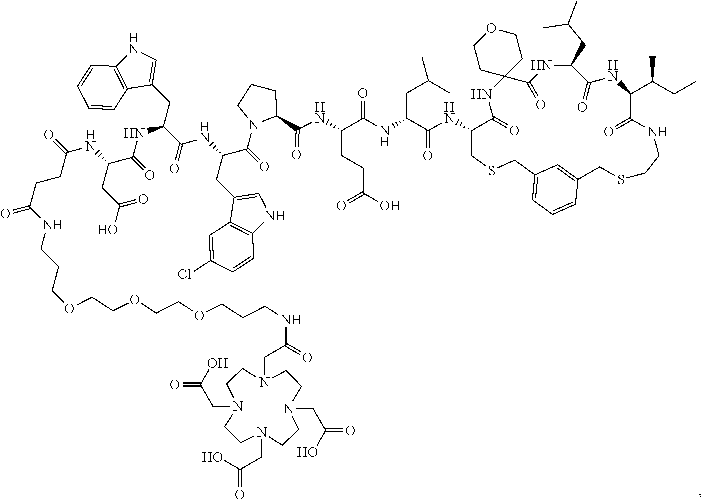 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2CCCN2C(=O)[C@H](Cc2c[nH]c3ccc(Cl)cc23)NC(=O)[C@H](Cc2c[nH]c3ccccc23)NC(=O)[C@H](CC(=O)O)NC(=O)CCC(=O)NCCCOCCOCCOCCCNC(=O)CN2CCN(CC(=O)O)CCN(CC(=O)O)CCN(CC(=O)O)CC2)CSCc2cccc(c2)CSCCNC1=O]

compound DOTA-Ttds-Asp-Trp-5Clw-Pro-Glu-leu-[Cys(3MeBn)-Apc-Leu-Ile-AET] (GIP-088, SEQ ID NO: 89) of the following formula

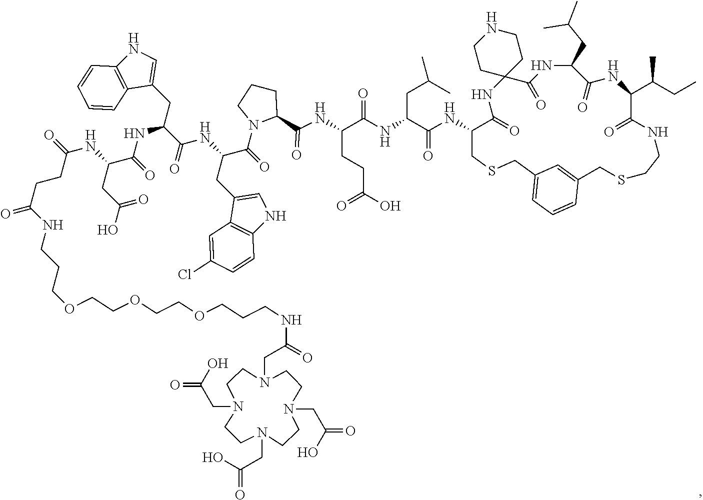 [SMILES: CC[C@H](C)[C@@H]1NC(=O)[C@H](CC(C)C)NC(=O)C2(CCNCC2)NC(=O)[C@@H](NC(=O)[C@@H](CC(C)C)NC(=O)[C@H](CCC(=O)O)NC(=O)[C@@H]2CCCN2C(=O)[C@H](Cc2c[nH]c3ccc(Cl)cc23)NC(=O)[C@H](Cc2c[nH]c3ccccc23)NC(=O)[C@H](CC(=O)O)NC(=O)CCC(=O)NCCCOCCOCCOCCCNC(=O)CN2CCN(CC(=O)O)CCN(CC(=O)O)CCN(CC(=O)O)CC2)CSCc2cccc(c2)CSCCNC1=O]

compound DOTA-APAc-Asp-Trp-5Clw-Pro-Glu-leu-[Cys(3MeBn)-Aib-Leu-Ile-AET] (GIP-089, SEQ TD NO: 90) of the following formula

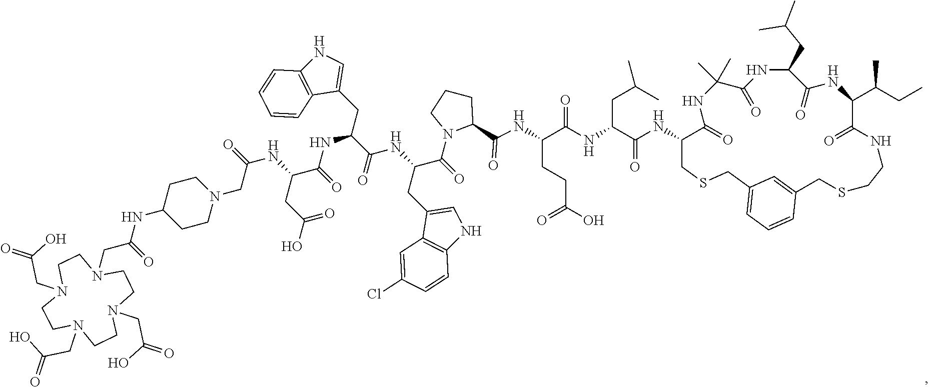 [SMILES: CC[C@H](C)[C@@H]1NC(=O)[C@H](CC(C)C)NC(=O)C(C)(C)NC(=O)[C@@H](NC(=O)[C@@H](CC(C)C)NC(=O)[C@H](CCC(=O)O)NC(=O)[C@@H]2CCCN2C(=O)[C@H](Cc2c[nH]c3ccc(Cl)cc23)NC(=O)[C@H](Cc2c[nH]c3ccccc23)NC(=O)[C@H](CC(=O)O)NC(=O)CN2CCC(NC(=O)CN3CCN(CC(=O)O)CCN(CC(=O)O)CCN(CC(=O)O)CC3)CC2)CSCc2cccc(c2)CSCCNC1=O]

compound DOTA-Ttds-Asp-Trp-5Clw-Pro-Glu-leu-[Cys(2Lut)-Aib-Leu-Ile-AET] (GIP-090, SEQ ID NO: 91) of the following formula

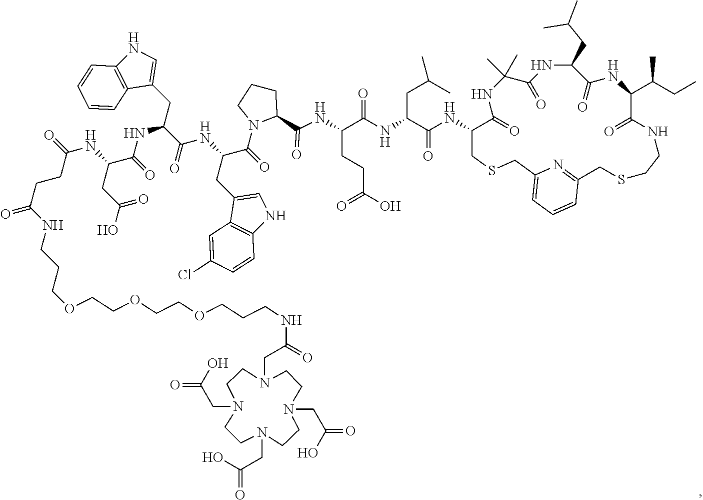 [SMILES: CC[C@H](C)[C@@H]1NC(=O)[C@H](CC(C)C)NC(=O)C(C)(C)NC(=O)[C@@H](NC(=O)[C@@H](CC(C)C)NC(=O)[C@H](CCC(=O)O)NC(=O)[C@@H]2CCCN2C(=O)[C@H](Cc2c[nH]c3ccc(Cl)cc23)NC(=O)[C@H](Cc2c[nH]c3ccccc23)NC(=O)[C@H](CC(=O)O)NC(=O)CCC(=O)NCCCOCCOCCOCCCNC(=O)CN2CCN(CC(=O)O)CCN(CC(=O)O)CCN(CC(=O)O)CC2)CSCc2cccc(n2)CSCCNC1=O]

compound DOTA-Ttds-Asp-Trp-5Clw-Pro-Glu-leu-[Cys(3Lut)-Aib-Leu-Ile-AET] (GIP-091, SEQ ID NO: 92) of the following formula

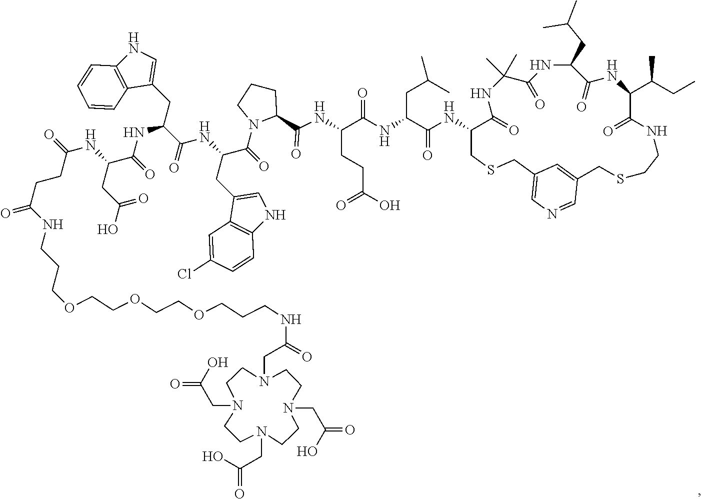 [SMILES: CC[C@H](C)[C@@H]1NC(=O)[C@H](CC(C)C)NC(=O)C(C)(C)NC(=O)[C@@H](NC(=O)[C@@H](CC(C)C)NC(=O)[C@H](CCC(=O)O)NC(=O)[C@@H]2CCCN2C(=O)[C@H](Cc2c[nH]c3ccc(Cl)cc23)NC(=O)[C@H](Cc2c[nH]c3ccccc23)NC(=O)[C@H](CC(=O)O)NC(=O)CCC(=O)NCCCOCCOCCOCCCNC(=O)CN2CCN(CC(=O)O)CCN(CC(=O)O)CCN(CC(=O)O)CC2)CSCc2cncc(c2)CSCCNC1=O]

compound DOTA-Ttds-Asp-Trp-5Clw-Pro-Glu-leu-[Dap(3MeBn)-Aib-Leu-Ile-AET] (GIP-092, SEQ ID NO: 93) of the following formula

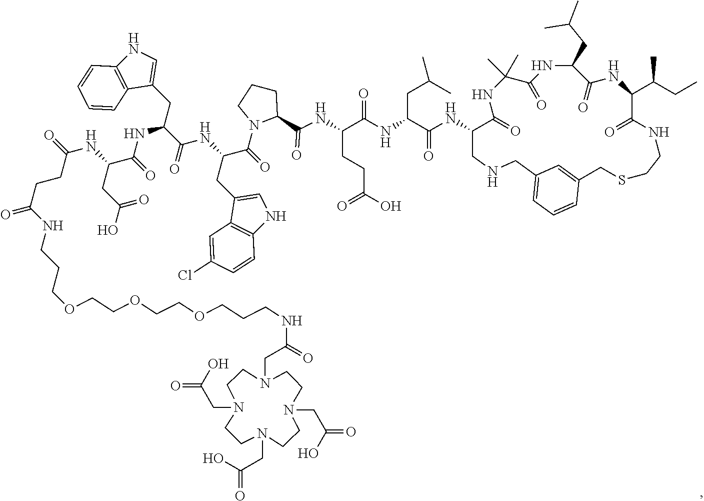 [SMILES: CC[C@H](C)[C@@H]1NC(=O)[C@H](CC(C)C)NC(=O)C(C)(C)NC(=O)[C@@H](NC(=O)[C@@H](CC(C)C)NC(=O)[C@H](CCC(=O)O)NC(=O)[C@@H]2CCCN2C(=O)[C@H](Cc2c[nH]c3ccc(Cl)cc23)NC(=O)[C@H](Cc2c[nH]c3ccccc23)NC(=O)[C@H](CC(=O)O)NC(=O)CCC(=O)NCCCOCCOCCOCCCNC(=O)CN2CCN(CC(=O)O)CCN(CC(=O)O)CCN(CC(=O)O)CC2)CNCc2cccc(c2)CSCCNC1=O]

compound DOTA-Ttds-Asp-Trp-5Clw-Pro-Glu-leu-[Cys(3MeBn)-Aib-Leu-Ile-en] (GIP-093, SEQ ID NO: 94) of the following formula

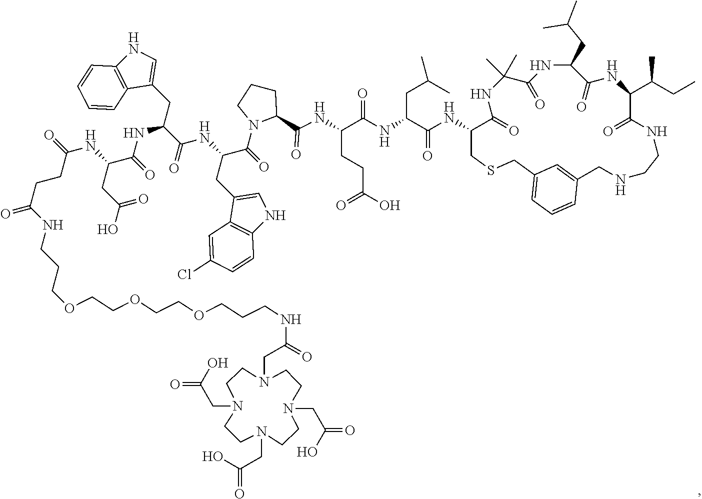 [SMILES: CC[C@H](C)[C@@H]1NC(=O)[C@H](CC(C)C)NC(=O)C(C)(C)NC(=O)[C@@H](NC(=O)[C@@H](CC(C)C)NC(=O)[C@H](CCC(=O)O)NC(=O)[C@@H]2CCCN2C(=O)[C@H](Cc2c[nH]c3ccc(Cl)cc23)NC(=O)[C@H](Cc2c[nH]c3ccccc23)NC(=O)[C@H](CC(=O)O)NC(=O)CCC(=O)NCCCOCCOCCOCCCNC(=O)CN2CCN(CC(=O)O)CCN(CC(=O)O)CCN(CC(=O)O)CC2)CSCc2cccc(c2)CNCCNC1=O]

compound DOTA-Ttds-Asp-Trp-5Clw-Hyp-Glu-leu-[Cys(3MeBn)-Aib-Leu-Ile-AET] (GIP-094, SEQ ID NO: 95) of the following formula

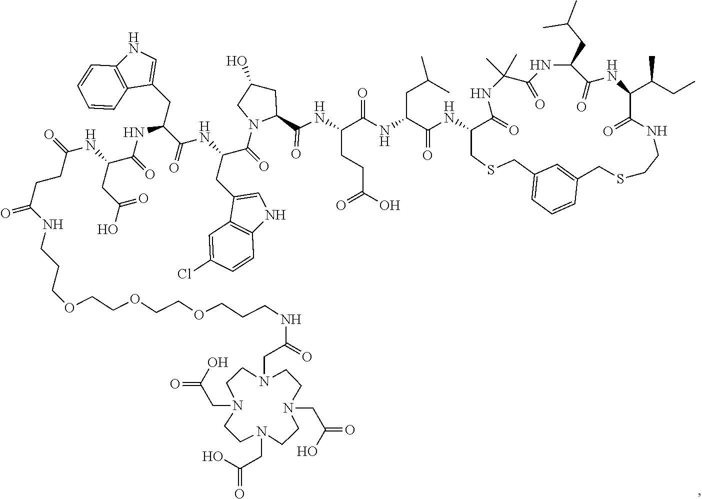 [SMILES: CC[C@H](C)[C@@H]1NC(=O)[C@H](CC(C)C)NC(=O)C(C)(C)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CC(=O)O)NC(=O)CCC(=O)NCCCOCCOCCOCCCNC(=O)CN2CCN(CC(=O)O)CCN(CC(=O)O)CCN(CC(=O)O)CC2)CSCc2cccc(c2)CSCCNC1=O]

compound DOTA-Ttds-Asp-Trp-5Clw-Tap-Glu-leu-[Cys(3MeBn)-Aib-Leu-Ile-AET] (GIP-095, SEQ ID NO: 96) of the following formula

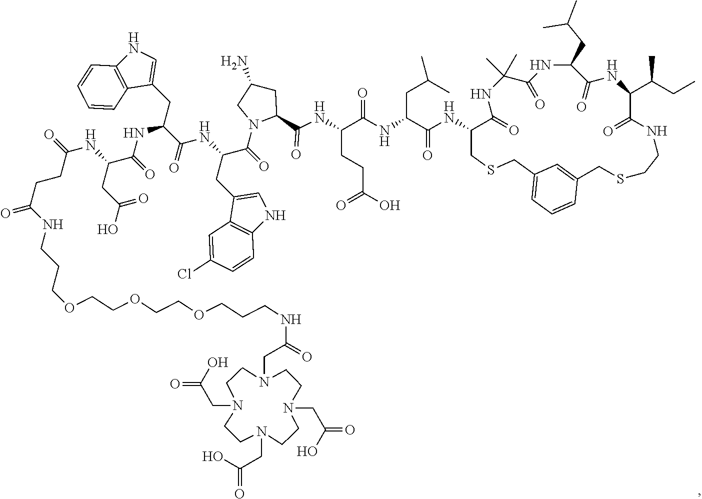 [SMILES: CC[C@H](C)[C@@H]1NC(=O)[C@H](CC(C)C)NC(=O)C(C)(C)NC(=O)[C@@H](NC(=O)[C@@H](CC(C)C)NC(=O)[C@H](CCC(=O)O)NC(=O)[C@@H]2C[C@@H](N)CN2C(=O)[C@H](Cc2c[nH]c3ccc(Cl)cc23)NC(=O)[C@H](Cc2c[nH]c3ccccc23)NC(=O)[C@H](CC(=O)O)NC(=O)CCC(=O)NCCCOCCOCCOCCCNC(=O)CN2CCN(CC(=O)O)CCN(CC(=O)O)CCN(CC(=O)O)CC2)CSCc2cccc(c2)CSCCNC1=O]

compound DOTA-Ttds-Asp-Trp-5Clw-4Ap-Glu-leu-[Cys(3MeBn)-Aib-Leu-Ile-AET] (GIP-096, SEQ ID NO: 97) of the following formula

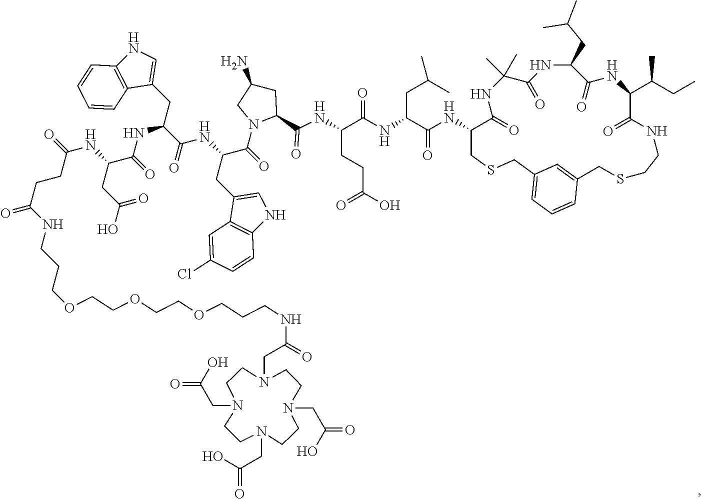 [SMILES: CC[C@H](C)[C@@H]1NC(=O)[C@H](CC(C)C)NC(=O)C(C)(C)NC(=O)[C@@H](NC(=O)[C@@H](CC(C)C)NC(=O)[C@H](CCC(=O)O)NC(=O)[C@@H]2C[C@H](N)CN2C(=O)[C@H](Cc2c[nH]c3ccc(Cl)cc23)NC(=O)[C@H](Cc2c[nH]c3ccccc23)NC(=O)[C@H](CC(=O)O)NC(=O)CCC(=O)NCCCOCCOCCOCCCNC(=O)CN2CCN(CC(=O)O)CCN(CC(=O)O)CCN(CC(=O)O)CC2)CSCc2cccc(c2)CSCCNC1=O]

compound DOTA-Ttds-Asp-Trp-5Clw-Pro-Glu-leu-[Cys(3MeBn)-Apc(Ac)-Leu-Ile-AET] (GIP-097, SEQ ID NO: 98) of the following formula

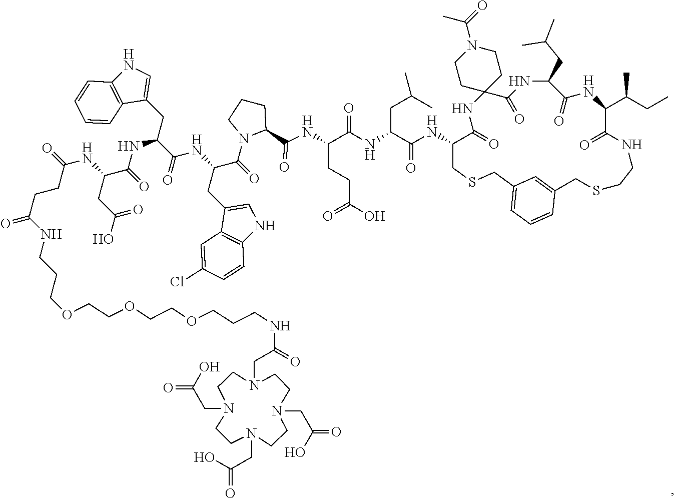 [SMILES: CC[C@H](C)[C@@H]1NC(=O)[C@H](CC(C)C)NC(=O)C2(CCN(C(C)=O)CC2)NC(=O)[C@@H](NC(=O)[C@@H](CC(C)C)NC(=O)[C@H](CCC(=O)O)NC(=O)[C@@H]2CCCN2C(=O)[C@H](Cc2c[nH]c3ccc(Cl)cc23)NC(=O)[C@H](Cc2c[nH]c3ccccc23)NC(=O)[C@H](CC(=O)O)NC(=O)CCC(=O)NCCCOCCOCCOCCCNC(=O)CN2CCN(CC(=O)O)CCN(CC(=O)O)CCN(CC(=O)O)CC2)CSCc2cccc(c2)CSCCNC1=O]

compound DOTA-Ttds-Asp-Trp-5Clw-Pro-Glu-leu-[Dap(3MeBn)-Aib-Leu-Ile-AETO] (GIP-098, SEQ ID NO: 99) of the following formula

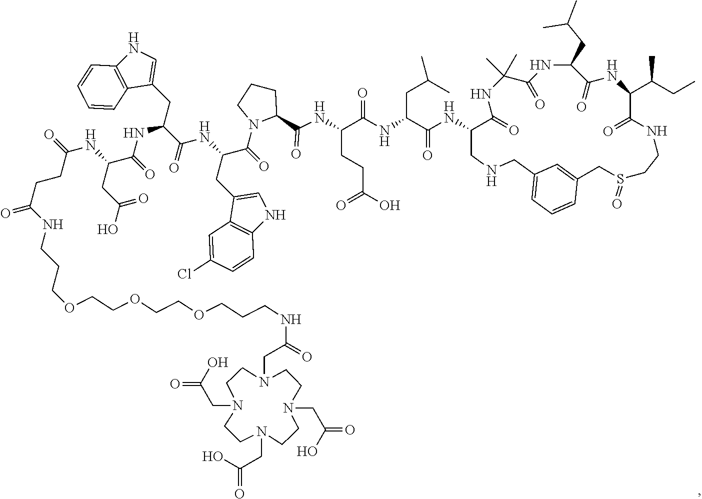 [SMILES: CC[C@H](C)[C@@H]1NC(=O)[C@H](CC(C)C)NC(=O)C(C)(C)NC(=O)[C@@H](NC(=O)[C@@H](CC(C)C)NC(=O)[C@H](CCC(=O)O)NC(=O)[C@@H]2CCCN2C(=O)[C@H](Cc2c[nH]c3ccc(Cl)cc23)NC(=O)[C@H](Cc2c[nH]c3ccccc23)NC(=O)[C@H](CC(=O)O)NC(=O)CCC(=O)NCCCOCCOCCOCCCNC(=O)CN2CCN(CC(=O)O)CCN(CC(=O)O)CCN(CC(=O)O)CC2)CNCc2cccc(c2)CS(=O)CCNC1=O]

compound DOTA-Ttds-Asp-Trp-5Clw-Pro-Glu-leu-[Cys(3MeBn)-Aib-Hle-Ile-AET] (GIP-099, SEQ ID NO: 100) of the following formula

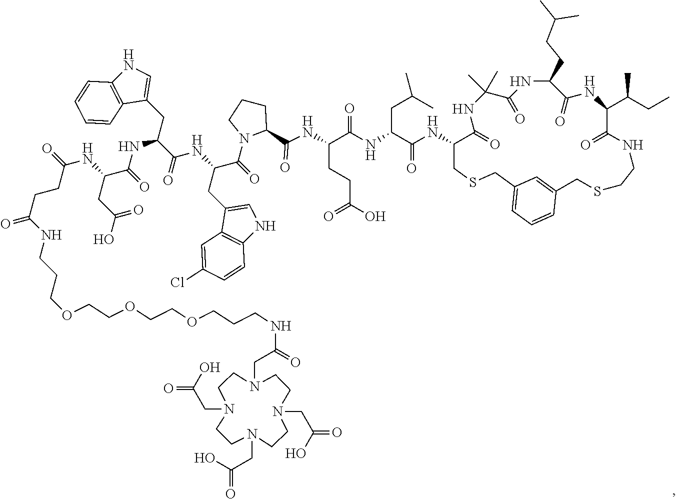 [SMILES: CC[C@H](C)[C@@H]1NC(=O)[C@H](CCC(C)C)NC(=O)C(C)(C)NC(=O)[C@@H](NC(=O)[C@@H](CC(C)C)NC(=O)[C@H](CCC(=O)O)NC(=O)[C@@H]2CCCN2C(=O)[C@H](Cc2c[nH]c3ccc(Cl)cc23)NC(=O)[C@H](Cc2c[nH]c3ccccc23)NC(=O)[C@H](CC(=O)O)NC(=O)CCC(=O)NCCCOCCOCCOCCCNC(=O)CN2CCN(CC(=O)O)CCN(CC(=O)O)CCN(CC(=O)O)CC2)CSCc2cccc(c2)CSCCNC1=O]

compound DOTA-Ttds-Asp-Trp-5Clw-Nmg-Glu-leu-[Cys(3MeBn)-Aib-Leu-Ile-AET] (GIP-100, SEQ ID NO: 101) of the following formula

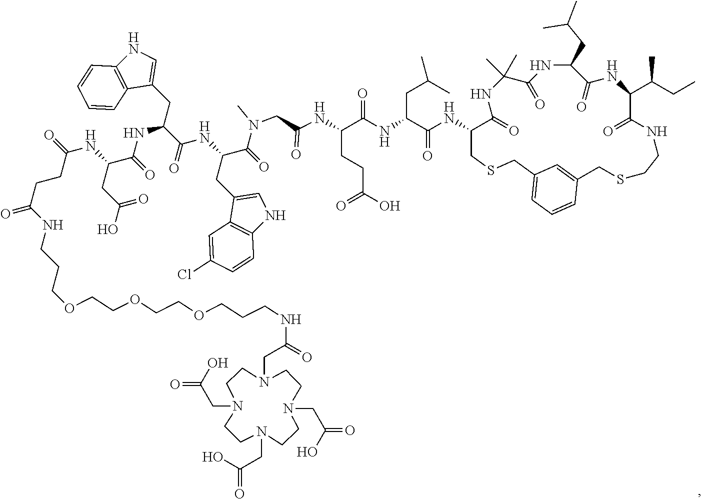

compound DOTA-Ttds-Asp-Trp-5Clw-Pro-Glu-leu-[Cys(3MeBn)-Ams-Leu-Ile-AET] (GIP-101, SEQ ID NO: 102) of the following formula

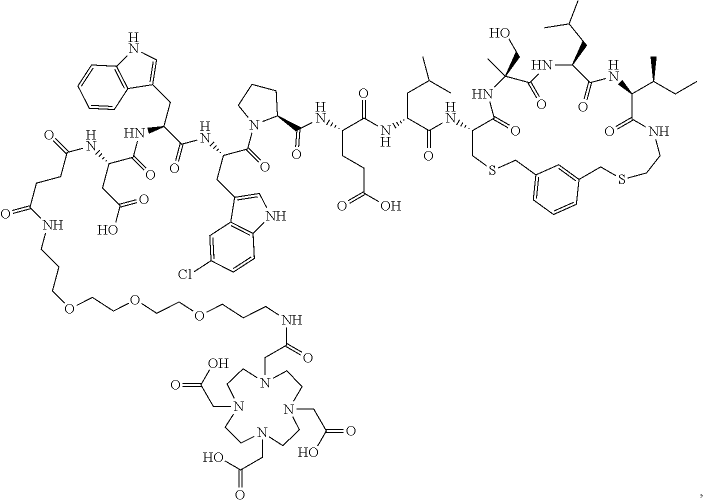 [SMILES: CC[C@H](C)[C@@H]1NC(=O)[C@H](CC(C)C)NC(=O)[C@](C)(CO)NC(=O)[C@@H](NC(=O)[C@@H](CC(C)C)NC(=O)[C@H](CCC(=O)O)NC(=O)[C@@H]2CCCN2C(=O)[C@H](Cc2c[nH]c3ccc(Cl)cc23)NC(=O)[C@H](Cc2c[nH]c3ccccc23)NC(=O)[C@H](CC(=O)O)NC(=O)CCC(=O)NCCCOCCOCCOCCCNC(=O)CN2CCN(CC(=O)O)CCN(CC(=O)O)CCN(CC(=O)O)CC2)CSCc2cccc(c2)CSCCNC1=O]

compound DOTA-Ttds-Asp-Trp-5Clw-H3p-Glu-leu-[Cys(3MeBn)-Aib-Leu-Ile-AET] (GIP-102, SEQ ID NO: 103) of the following formula

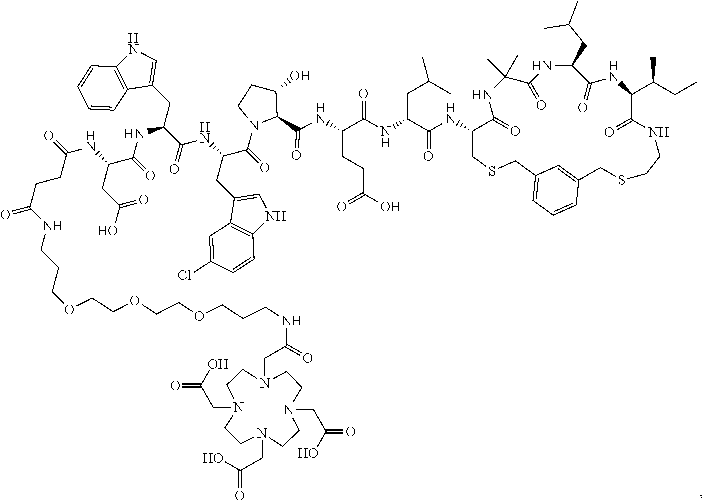 [SMILES: CC[C@H](C)[C@@H]1NC(=O)[C@H](CC(C)C)NC(=O)C(C)(C)NC(=O)[C@@H](NC(=O)[C@@H](CC(C)C)NC(=O)[C@H](CCC(=O)O)NC(=O)[C@@H]2[C@@H](O)CCN2C(=O)[C@H](Cc2c[nH]c3ccc(Cl)cc23)NC(=O)[C@H](Cc2c[nH]c3ccccc23)NC(=O)[C@H](CC(=O)O)NC(=O)CCC(=O)NCCCOCCOCCOCCCNC(=O)CN2CCN(CC(=O)O)CCN(CC(=O)O)CCN(CC(=O)O)CC2)CSCc2cccc(c2)CSCCNC1=O]

compound DOTA-APAc-Asp-Trp-5Clw-Hyp-Glu-leu-[Cys(2Lut)-Thp-Leu-Ile-AET] (GIP-103, SEQ ID NO: 104) of the following formula

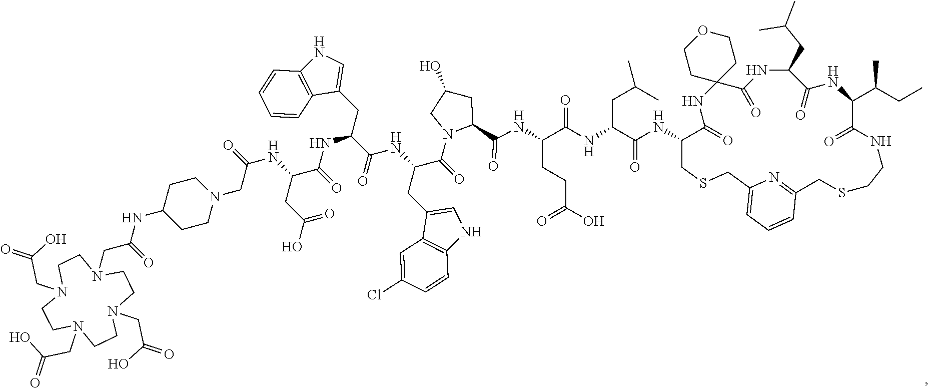 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CC(=O)O)NC(=O)CN2CCC(NC(=O)CN3CCN(CC(=O)O)CCN(CC(=O)O)CCN(CC(=O)O)CC3)CC2)CSCc2cccc(n2)CSCCNC1=O]

compound DOTA-Ttds-Asp-6Fw-5Clw-Pro-Glu-leu-[Cys(3MeBn)-Aib-Leu-Ile-AET] (GIP-104, SEQ ID NO: 105) of the following formula

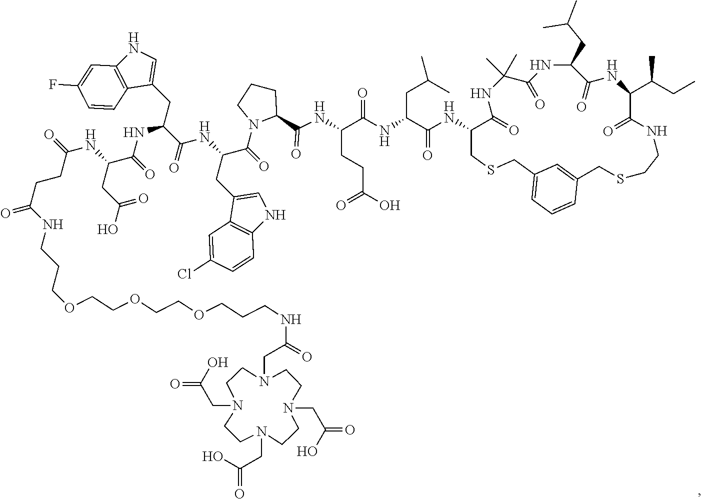 [SMILES: CC[C@H](C)[C@@H]1NC(=O)[C@H](CC(C)C)NC(=O)C(C)(C)NC(=O)[C@@H](NC(=O)[C@@H](CC(C)C)NC(=O)[C@H](CCC(=O)O)NC(=O)[C@@H]2CCCN2C(=O)[C@H](Cc2c[nH]c3ccc(Cl)cc23)NC(=O)[C@H](Cc2c[nH]c3cc(F)ccc23)NC(=O)[C@H](CC(=O)O)NC(=O)CCC(=O)NCCCOCCOCCOCCCNC(=O)CN2CCN(CC(=O)O)CCN(CC(=O)O)CCN(CC(=O)O)CC2)CSCc2cccc(c2)CSCCNC1=O]

compound DOTA-Ttds-Asp-6Fw-5Brw-Pro-Glu-leu-[Cys(3MeBn)-Aib-Leu-Ile-AET] (GIP-105, SEQ ID NO: 106) of the following formula

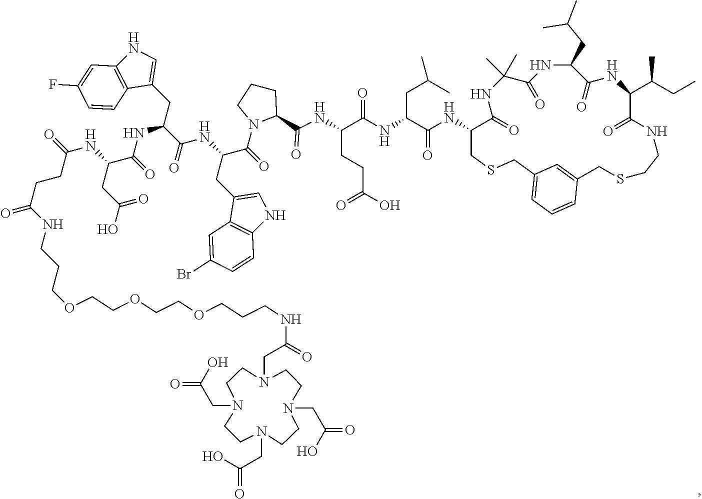 [SMILES: CC[C@H](C)[C@@H]1NC(=O)[C@H](CC(C)C)NC(=O)C(C)(C)NC(=O)[C@@H](NC(=O)[C@@H](CC(C)C)NC(=O)[C@H](CCC(=O)O)NC(=O)[C@@H]2CCCN2C(=O)[C@H](Cc2c[nH]c3ccc(Br)cc23)NC(=O)[C@H](Cc2c[nH]c3cc(F)ccc23)NC(=O)[C@H](CC(=O)O)NC(=O)CCC(=O)NCCCOCCOCCOCCCNC(=O)CN2CCN(CC(=O)O)CCN(CC(=O)O)CCN(CC(=O)O)CC2)CSCc2cccc(c2)CSCCNC1=O]

compound DOTA-APAc-Asp-Trp-5Clw-Hyp-Glu-leu-[Dap(2Lut)-Thp-Leu-Ile-AET] (GIP-106, SEQ ID NO: 107) of the following formula

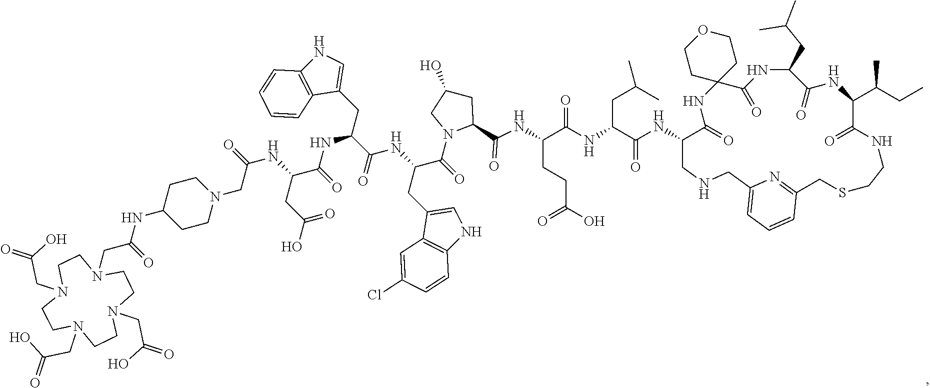 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CC(=O)O)NC(=O)CN2CCC(NC(=O)CN3CCN(CC(=O)O)CCN(CC(=O)O)CCN(CC(=O)O)CC3)CC2)CNCc2cccc(n2)CSCCNC1=O]

compound DOTA-APAc-Asp-Trp-5Clw-Hyp-Glu-leu-[Dap(3MeBn)-Thp-Leu-Ile-AET] (GIP-107, SEQ ID NO: 108) of the following formula

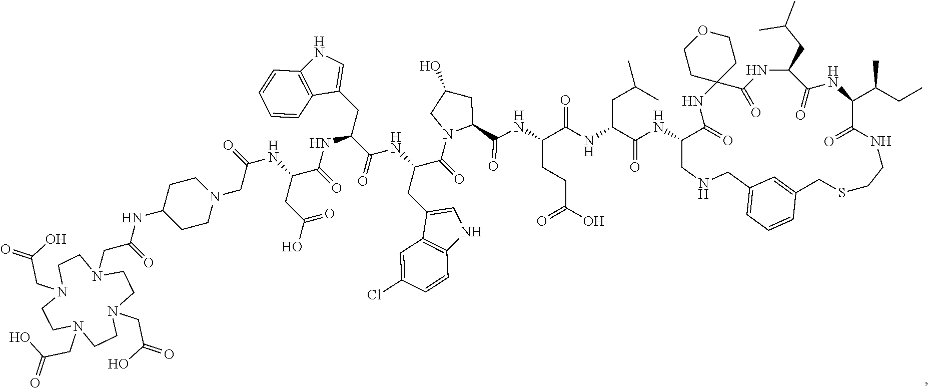 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CC(=O)O)NC(=O)CN2CCC(NC(=O)CN3CCN(CC(=O)O)CCN(CC(=O)O)CCN(CC(=O)O)CC3)CC2)CNCc2cccc(c2)CSCCNC1=O]

compound HO-Succinyl-Trp-5Clw-Hyp-Glu-leu-[Cys(2Lut)-Thp-Leu-Ile-Cys]-Ttds-Lys(DOTA)-NH₂ (GIP-108, SEQ ID N: 109) of the following formula

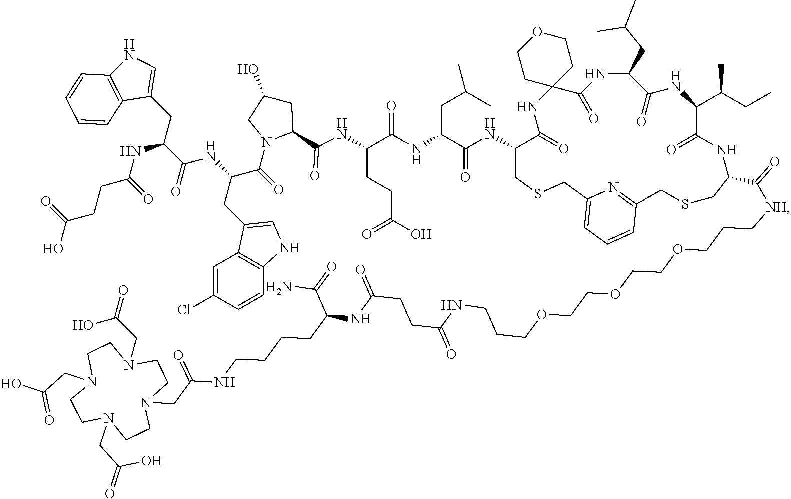 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)CCC(=O)O)CSCc2cccc(n2)CSC[C@@H](C(=O)NCCCOCCOCCOCCCNC(=O)CCC(=O)N[C@@H](CCCCNC(=O)CN2CCN(CC(=O)O)CCN(CC(=O)O)CCN(CC(=O)O)CC2)C(N)=O)NC1=O]

compound HO-Succinyl-Trp-5Clw-Hyp-Glu-leu-[Cys(2Lut)-Thp-Leu-Ile-Cys]-APAc-Lys(DOTA)-NH₂ (GIP-109, SEQ ID NO: 110) of the following formula

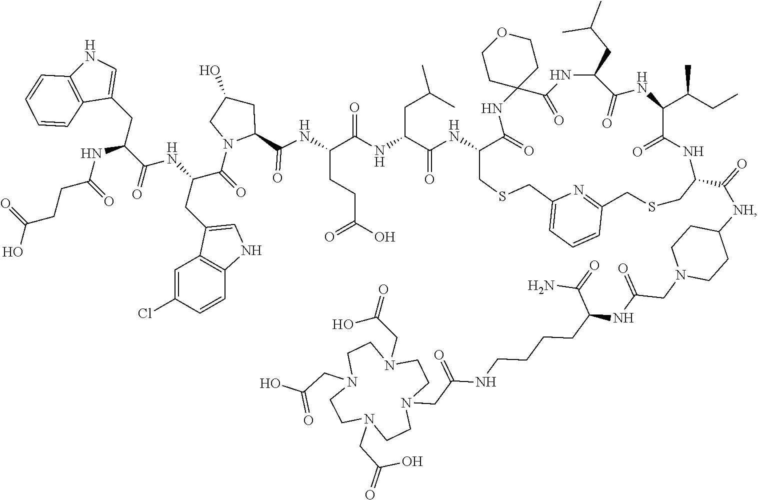 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)CCC(=O)O)CSCc2cccc(n2)CSC[C@@H](C(=O)NC2CCN(CC(=O)N[C@@H](CCCCNC(=O)CN3CCN(CC(=O)O)CCN(CC(=O)O)CCN(CC(=O)O)CC3)C(N)=O)CC2)NC1=O]

compound HO-Succinyl-Trp-5Clw-Hyp-Glu-leu-[Cys(2Lut)-Apc(DOTA)-Leu-Ile-AET] (GIP-110, SEQ ID NO: 111) of the following formula

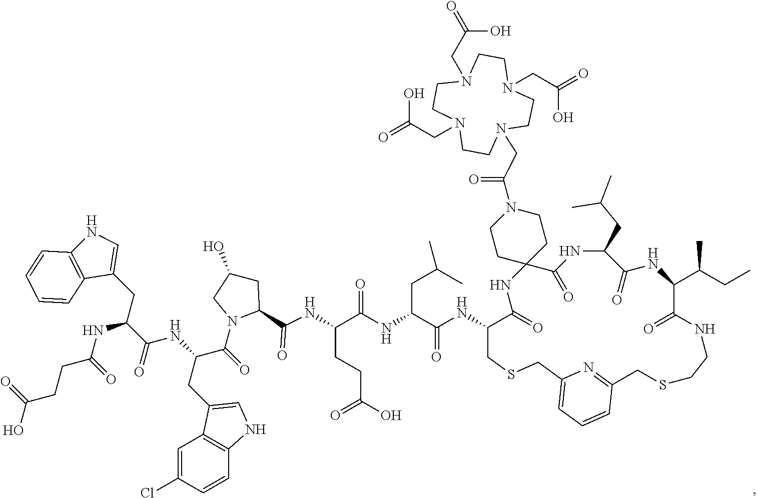 [SMILES: CC[C@H](C)[C@@H]1NC(=O)[C@H](CC(C)C)NC(=O)C2(CCN(C(=O)CN3CCN(CC(=O)O)CCN(CC(=O)O)CCN(CC(=O)O)CC3)CC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)CCC(=O)O)CSCc2cccc(n2)CSCCNC1=O]

compound DOTA-Ttds-Asp-Trp-5Clw-4Ap(Ac)-Glu-leu-[Cys(3MeBn)-Aib-Leu-Ile-AET] (GIP-111, SEQ ID NO: 112) of the following formula

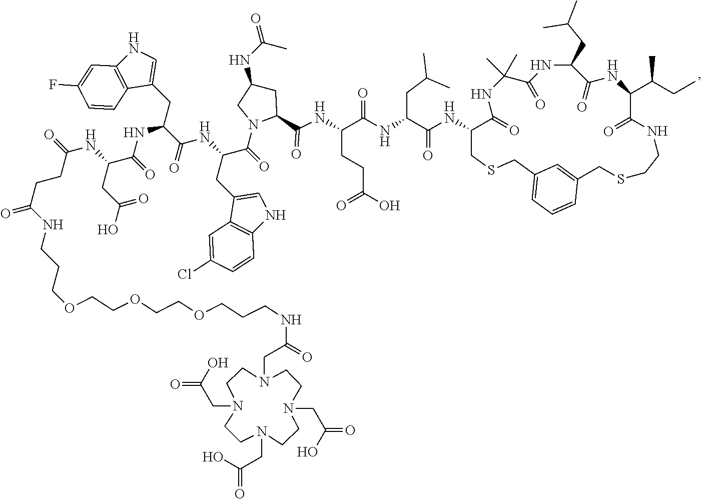 [SMILES: CC[C@H](C)[C@@H]1NC(=O)[C@H](CC(C)C)NC(=O)C(C)(C)NC(=O)[C@@H](NC(=O)[C@@H](CC(C)C)NC(=O)[C@H](CCC(=O)O)NC(=O)[C@@H]2C[C@H](NC(C)=O)CN2C(=O)[C@H](Cc2c[nH]c3ccc(Cl)cc23)NC(=O)[C@H](Cc2c[nH]c3cc(F)ccc23)NC(=O)[C@H](CC(=O)O)NC(=O)CCC(=O)NCCCOCCOCCOCCCNC(=O)CN2CCN(CC(=O)O)CCN(CC(=O)O)CCN(CC(=O)O)CC2)CSCc2cccc(c2)CSCCNC1=O]

compound DOTA-Ttds-Asp-Trp-5Clw-Tap-Glu-leu-[Cys(2Lut)-Apc-Leu-Ile-AET] (GIP-112, SEQ ID NO: 113) of the following formula

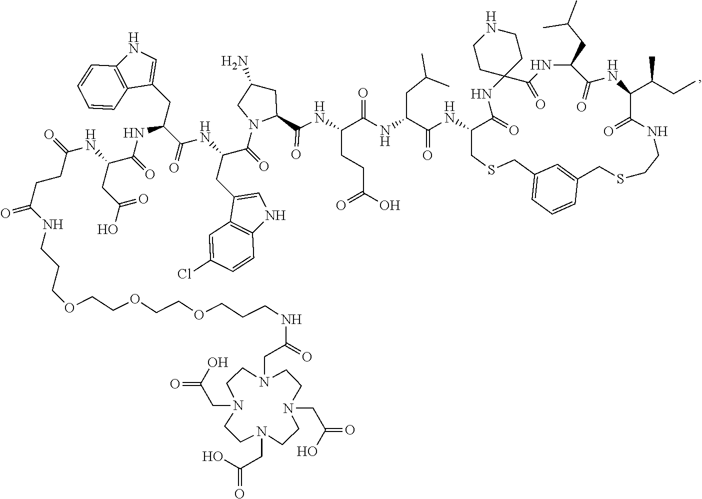 [SMILES: CC[C@H](C)[C@@H]1NC(=O)[C@H](CC(C)C)NC(=O)C2(CCNCC2)NC(=O)[C@@H](NC(=O)[C@@H](CC(C)C)NC(=O)[C@H](CCC(=O)O)NC(=O)[C@@H]2C[C@@H](N)CN2C(=O)[C@H](Cc2c[nH]c3ccc(Cl)cc23)NC(=O)[C@H](Cc2c[nH]c3ccccc23)NC(=O)[C@H](CC(=O)O)NC(=O)CCC(=O)NCCCOCCOCCOCCCNC(=O)CN2CCN(CC(=O)O)CCN(CC(=O)O)CCN(CC(=O)O)CC2)CSCc2cccc(c2)CSCCNC1=O]

compound DOTA-Ttds-Asp-Trp-5Clw-Tap-Glu-leu-[Dap(2Lut)-Apc-Leu-Ile-AET] (GIP-113, SEQ ID NO: 114) of the following formula

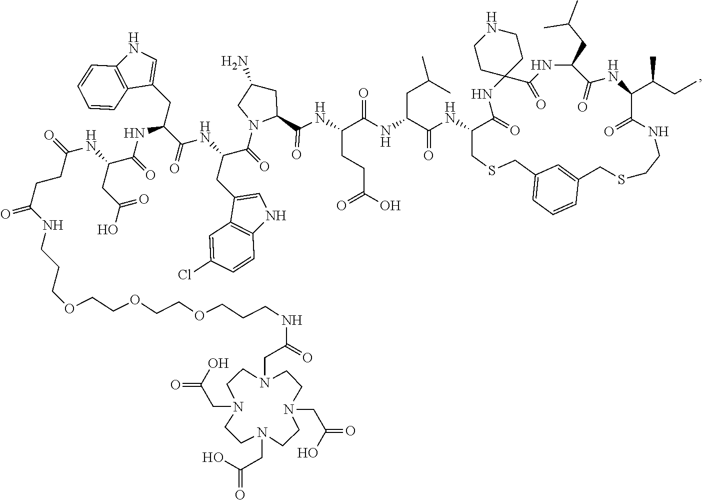 [SMILES: CC[C@H](C)[C@@H]1NC(=O)[C@H](CC(C)C)NC(=O)C2(CCNCC2)NC(=O)[C@@H](NC(=O)[C@@H](CC(C)C)NC(=O)[C@H](CCC(=O)O)NC(=O)[C@@H]2C[C@@H](N)CN2C(=O)[C@H](Cc2c[nH]c3ccc(Cl)cc23)NC(=O)[C@H](Cc2c[nH]c3ccccc23)NC(=O)[C@H](CC(=O)O)NC(=O)CCC(=O)NCCCOCCOCCOCCCNC(=O)CN2CCN(CC(=O)O)CCN(CC(=O)O)CCN(CC(=O)O)CC2)CSCc2cccc(c2)CSCCNC1=O]

compound HO-Succinyl-Trp-5Clw-Hyp-Glu-leu-[Cys(2Lut)-Apc-Leu-Ile-Cys]-APAc-Lys(DOTA)-NH₂ (GIP-114, SEQ ID NO: 115) of the following formula

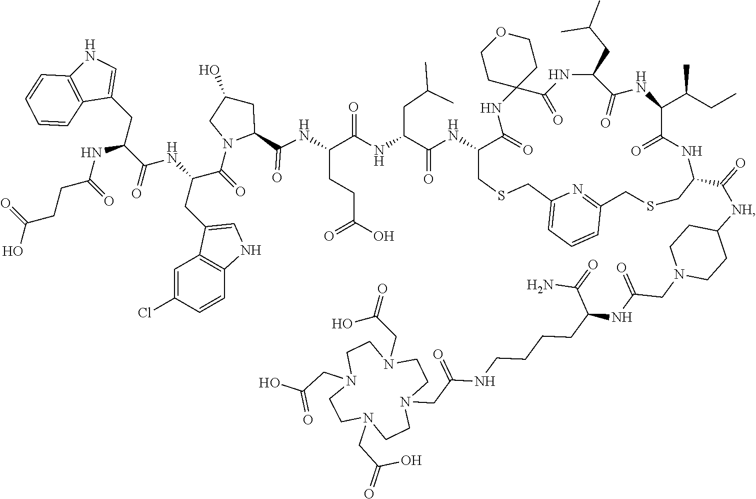 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)CCC(=O)O)CSCc2cccc(n2)CSC[C@@H](C(=O)NC2CCN(CC(=O)N[C@@H](CCCCNC(=O)CN3CCN(CC(=O)O)CCN(CC(=O)O)CCN(CC(=O)O)CC3)C(N)=O)CC2)NC1=O]

compound HO-Succinyl-Trp-5Clw-Hyp-Glu-leu-[Cys(2Lut)-Thp-Leu-Ile-Cys]-Lys(DOTA)-NH₂ (GIP-115, SEQ ID NO: 116) of the following formula

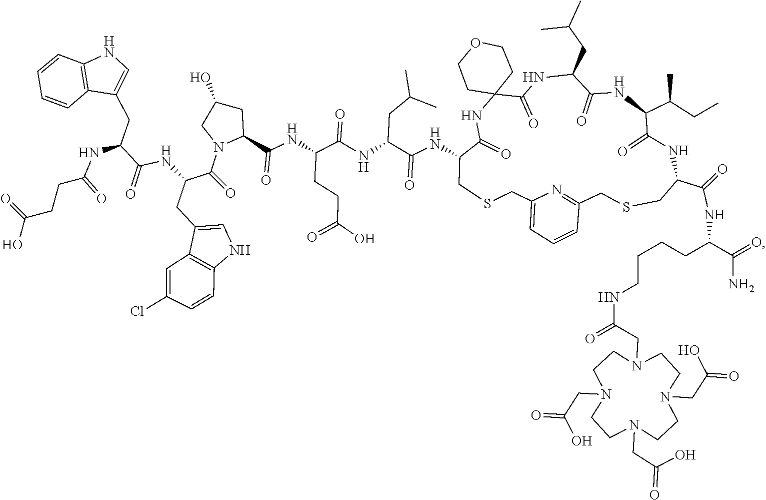 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)CCC(=O)O)CSCc2cccc(n2)CSC[C@@H](C(=O)N[C@@H](CCCCNC(=O)CN2CCN(CC(=O)O)CCN(CC(=O)O)CCN(CC(=O)O)CC2)C(N)=O)NC1=O]

compound HO-Succinyl-Trp-5Clw-Hyp-Glu-leu-[Cys(2Lut)-Thp-Leu-Ile-Cys]-lys(DOTA)-NH₂ (GIP-116, SEQ ID NO: 117) of the following formula

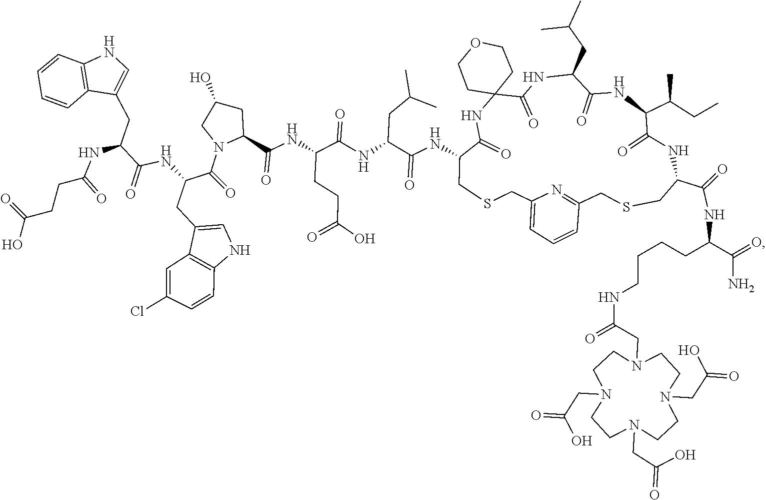 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)CCC(=O)O)CSCc2cccc(n2)CSC[C@@H](C(=O)N[C@H](CCCCNC(=O)CN2CCN(CC(=O)O)CCN(CC(=O)O)CCN(CC(=O)O)CC2)C(N)=O)NC1=O]

compound HO-Succinyl-Trp-5Clw-Hyp-Glu-leu-[Cys(2Lut)-Thp-Leu-Ile-Cys]-Ape-DOTA (GIP-117, SEQ ID NO: 118) of the following formula

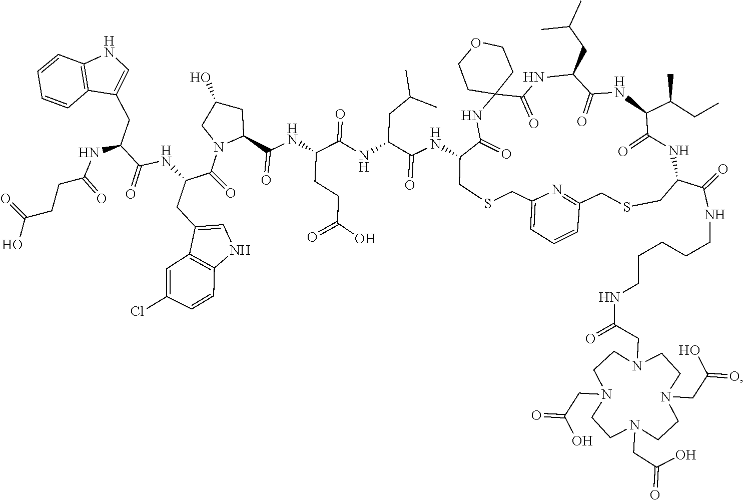 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)CCC(=O)O)CSCc2cccc(n2)CSC[C@@H](C(=O)NCCCCCNC(=O)CN2CCN(CC(=O)O)CCN(CC(=O)O)CCN(CC(=O)O)CC2)NC1=O]

compound HO-Succinyl-Trp-5Clw-Hyp-Glu-leu-[Cys(2Lut)-Thp-Leu-Ile-Cys]-PP-DOTA (GIP-118, SEQ ID NO: 119) of the following formula

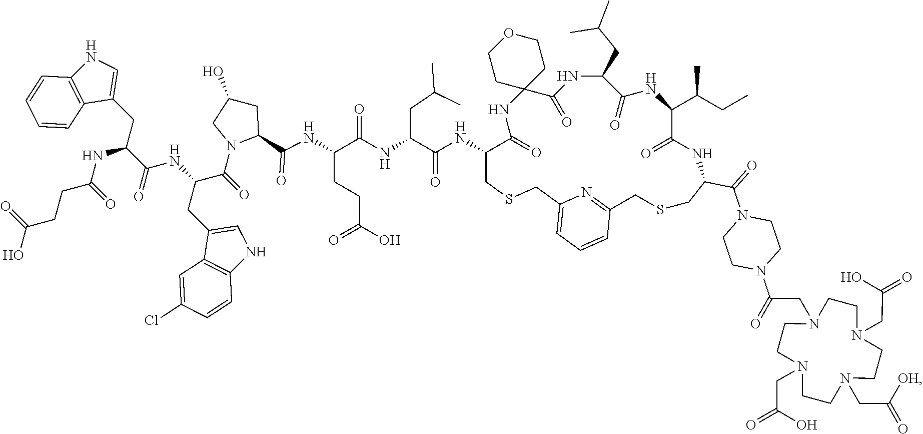 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)CCC(=O)O)CSCc2cccc(n2)CSC[C@@H](C(=O)N2CCN(C(=O)CN3CCN(CC(=O)O)CCN(CC(=O)O)CCN(CC(=O)O)CC3)CC2)NC1=O]

compound DOTA-Ttds-Asp-Trp-5Clw-Pro-Glu-leu-[Cys(2Lut)-Apc-Leu-Ile-AET] (GIP-119, SEQ ID NO: 120) of the following formula

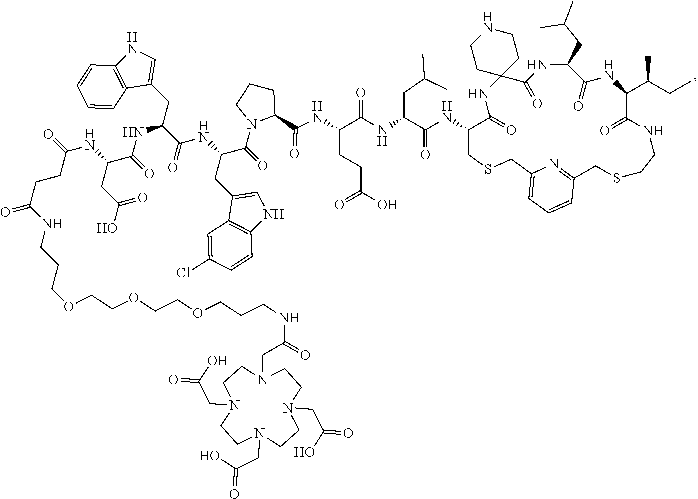 [SMILES: CC[C@H](C)[C@@H]1NC(=O)[C@H](CC(C)C)NC(=O)C2(CCNCC2)NC(=O)[C@@H](NC(=O)[C@@H](CC(C)C)NC(=O)[C@H](CCC(=O)O)NC(=O)[C@@H]2CCCN2C(=O)[C@H](Cc2c[nH]c3ccc(Cl)cc23)NC(=O)[C@H](Cc2c[nH]c3ccccc23)NC(=O)[C@H](CC(=O)O)NC(=O)CCC(=O)NCCCOCCOCCOCCCNC(=O)CN2CCN(CC(=O)O)CCN(CC(=O)O)CCN(CC(=O)O)CC2)CSCc2cccc(n2)CSCCNC1=O]

compound MeO2kDaPEGAc-Asp-Trp-5Clw-Hyp-Glu-leu-[Cys(2Lut)-Thp-Leu-Ile-Cys]-lys(DOTA)-NH₂ (GIP-120, SEQ ID NO: 121) of the following formula

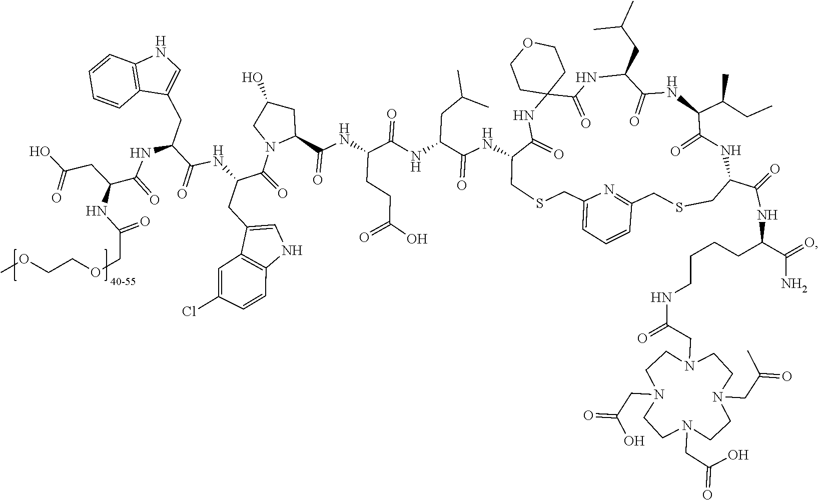

compound Ac-Asp-Trp-5Clw-Tap(DOTA)-Glu-leu-[Cys(2Lut)-Thp-Leu-Ile-Cys]-NH₂ (GIP-121, SED ID NO: 122) of the following formula

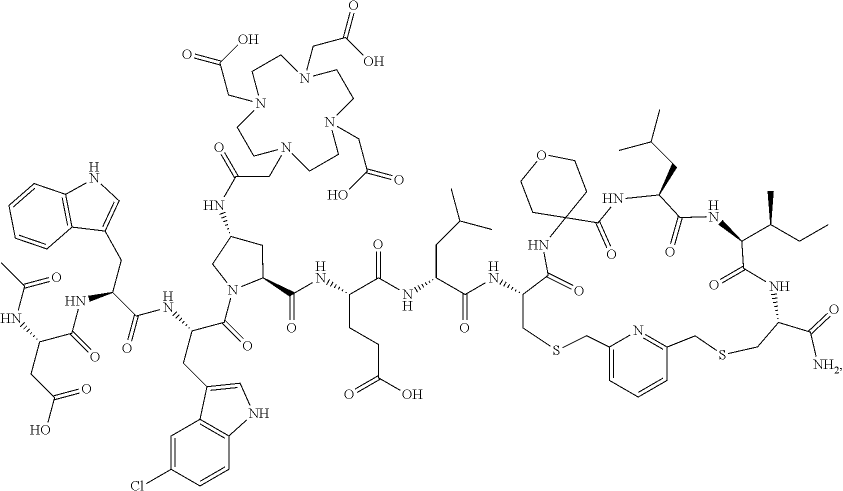 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](NC(=O)CN3CCN(CC(=O)O)CCN(CC(=O)O)CCN(CC(=O)O)CC3)CN2C(=O)[C@H](Cc2c[nH]c3ccc(Cl)cc23)NC(=O)[C@H](Cc2c[nH]c3ccccc23)NC(=O)[C@H](CC(=O)O)NC(C)=O)CSCc2cccc(n2)CSC[C@@H](C(N)=O)NC1=O]

compound Ac-Asp-Trp-5Clw-Hyp-Glu-leu-[Cys(2Lut)-Apc(DOTA)-Leu-Ile-Cys]-NH₂ (GIP-122, SEQ ID NO: 123) of the following formula

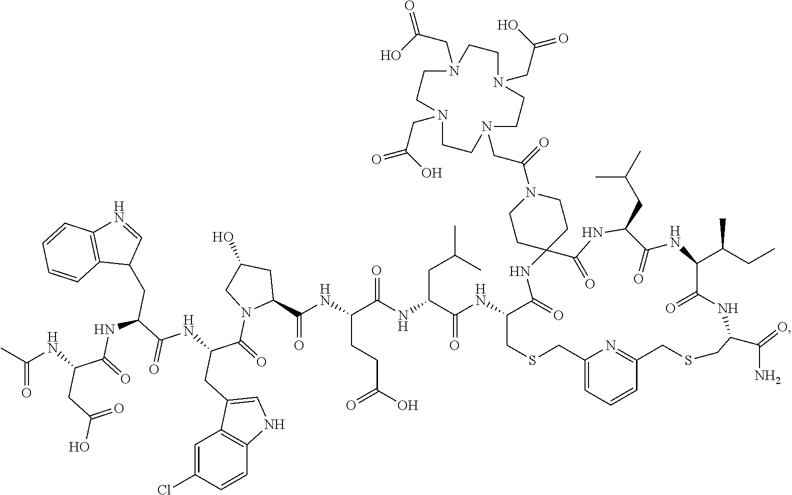

compound Ac-Asp-Trp-5Clw-Tap-Glu-leu-[Cys(2Lut)-Apc(DOTA)-Leu-Ile-Cys]-NH₂ (GIP-123, SEQ ID NO: 124) of the following formula

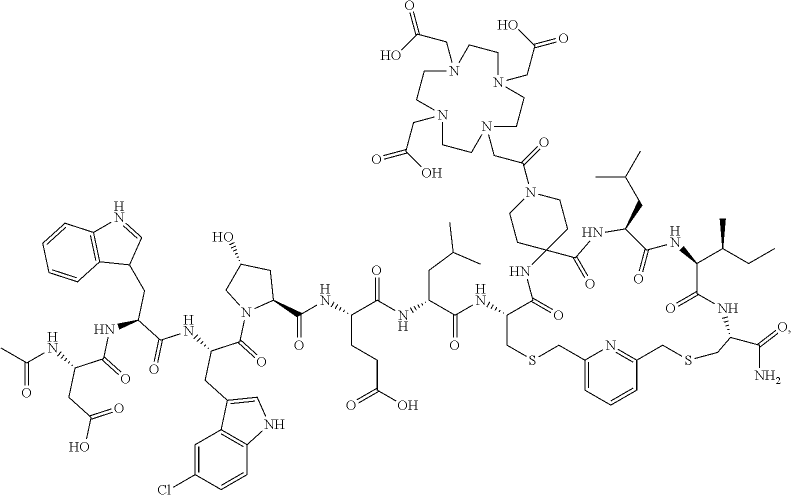

compound Ac-Asp-Trp-5Clw-Hyp-Glu-leu-[Cys(tMeBn(DOTA-AET))-Thp-Leu-Ile-Cys]-NH₂ (GIP-124, SEQ ID NO: 125) of the following formula

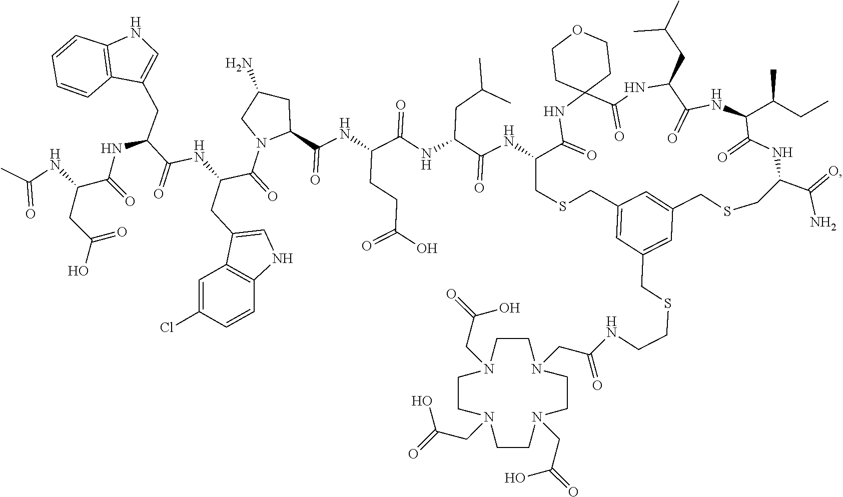 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](N)CN2C(=O)[C@H](Cc2c[nH]c3ccc(Cl)cc23)NC(=O)[C@H](Cc2c[nH]c3ccccc23)NC(=O)[C@H](CC(=O)O)NC(C)=O)CSCc2cc(CSCCNC(=O)CN3CCN(CC(=O)O)CCN(CC(=O)O)CCN(CC(=O)O)CC3)cc(c2)CSC[C@@H](C(N)=O)NC1=O]

compound DOTA-Asp-Trp-5Clw-Hyp-Glu-leu-[Cys(2Lut)-Thp-Leu-Ile-Cys]-NH₂ (GIP-125, SEQ ID NO: 126) of the following formula

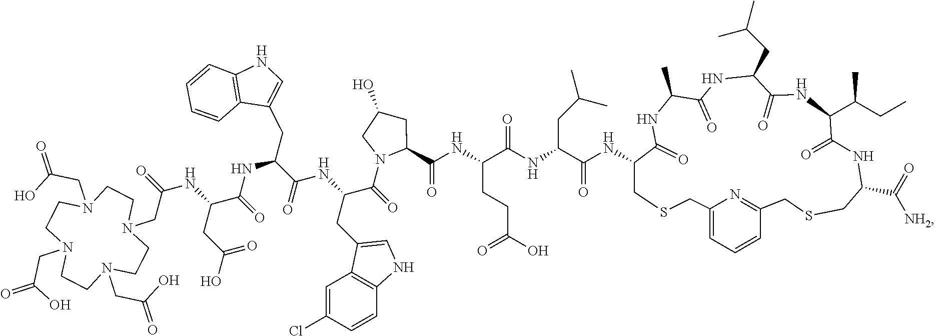 [SMILES: CC[C@H](C)[C@@H]1NC(=O)[C@H](CC(C)C)NC(=O)[C@H](C)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CC(=O)O)NC(=O)CN2CCN(CC(=O)O)CCN(CC(=O)O)CCN(CC(=O)O)CC2)CSCc2cccc(n2)CSC[C@@H](C(N)=O)NC1=O]

compound DOTA-Ttds-Tyr-Asp-Trp-Trp-Pro-Glu-Leu-[Cys(3MeBn)-Ala-Leu-Ile-Cys]-NH₂ (GIP-126, SEQ ID NO: 127) of the following formula

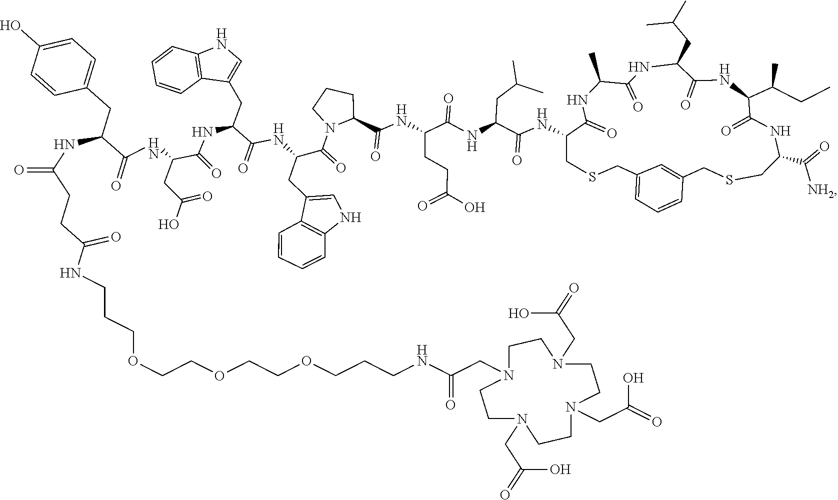 [SMILES: CC[C@H](C)[C@@H]1NC(=O)[C@H](CC(C)C)NC(=O)[C@H](C)NC(=O)[C@@H](NC(=O)[C@H](CC(C)C)NC(=O)[C@H](CCC(=O)O)NC(=O)[C@@H]2CCCN2C(=O)[C@H](Cc2c[nH]c3ccccc23)NC(=O)[C@H](Cc2c[nH]c3ccccc23)NC(=O)[C@H](CC(=O)O)NC(=O)[C@H](Cc2ccc(O)cc2)NC(=O)CCC(=O)NCCCOCCOCCOCCCNC(=O)CN2CCN(CC(=O)O)CCN(CC(=O)O)CCN(CC(=O)O)CC2)CSCc2cccc(c2)CSC[C@@H](C(N)=O)NC1=O]

compound Ac-Tyr-Asp-Trp-Trp-Pro-Glu-Leu-[Cys(3MeBn)-Ala-Leu-Ile-Cys]-Ttds-Lys(DOTA)-NH₂ (GIP-127, SEQ ID NO: 128) of the following formula

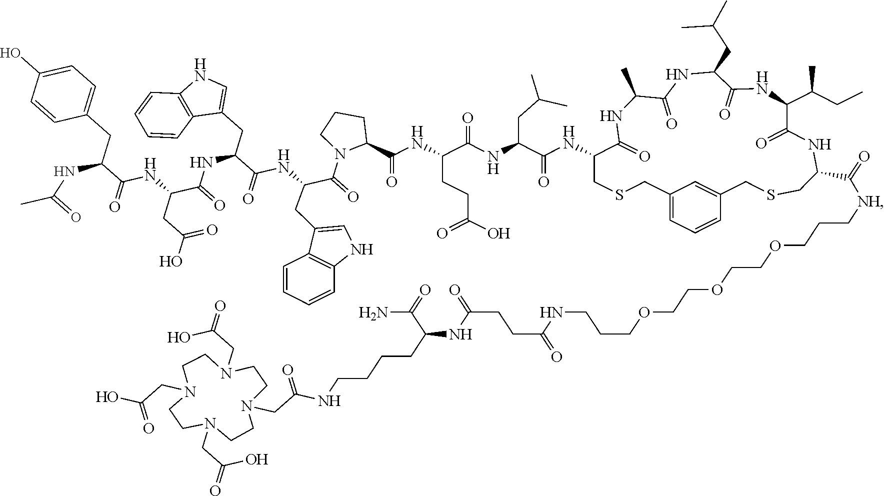 [SMILES: CC[C@H](C)[C@@H]1NC(=O)[C@H](CC(C)C)NC(=O)[C@H](C)NC(=O)[C@@H](NC(=O)[C@H](CC(C)C)NC(=O)[C@H](CCC(=O)O)NC(=O)[C@@H]2CCCN2C(=O)[C@H](Cc2c[nH]c3ccccc23)NC(=O)[C@H](Cc2c[nH]c3ccccc23)NC(=O)[C@H](CC(=O)O)NC(=O)[C@H](Cc2ccc(O)cc2)NC(C)=O)CSCc2cccc(c2)CSC[C@@H](C(=O)NCCCOCCOCCOCCCNC(=O)CCC(=O)N[C@@H](CCCCNC(=O)CN2CCN(CC(=O)O)CCN(CC(=O)O)CCN(CC(=O)O)CC2)C(N)=O)NC1=O]

compound DOTA-Ttds-Asp-Trp-Trp-Pro-Glu-leu-[Cys(3MeBn)-Aib-Leu-Ile-Cys]-NH₂ (GIP-128, SEQ ID NO: 129) of the following formula

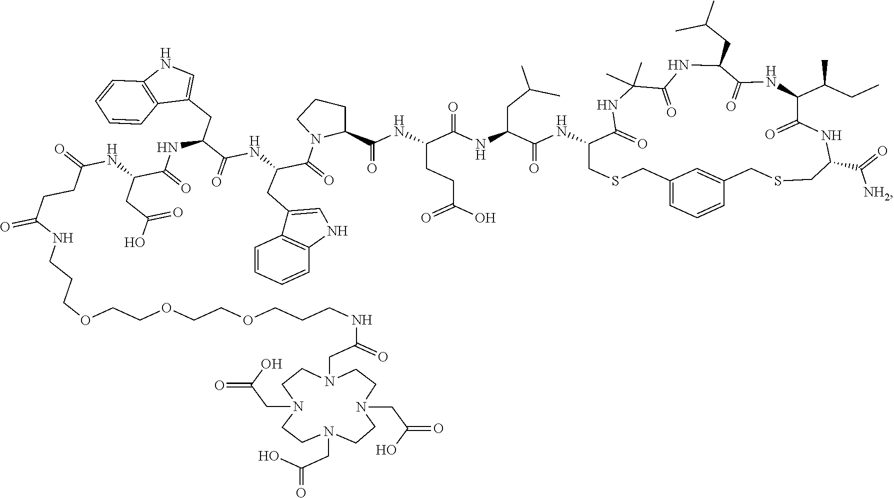

compound DOTA-Ttds-Asp-Trp-Trp-Pro-Glu-leu-[Cys(3MeBn)-Aib-Leu-Ile-AET] (GIP-129, SEQ ID NO: 130) of the following formula

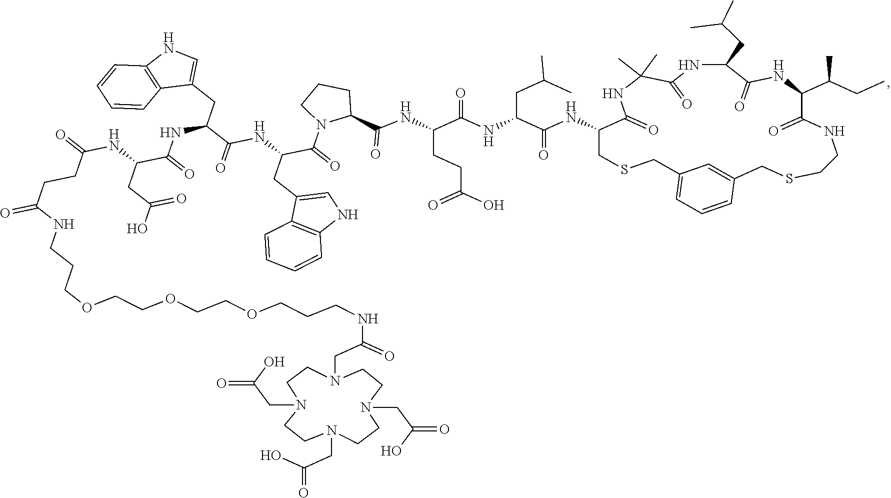 [SMILES: CC[C@H](C)[C@@H]1NC(=O)[C@H](CC(C)C)NC(=O)C(C)(C)NC(=O)[C@@H](NC(=O)[C@@H](CC(C)C)NC(=O)[C@H](CCC(=O)O)NC(=O)[C@@H]2CCCN2C(=O)[C@H](Cc2c[nH]c3ccccc23)NC(=O)[C@H](Cc2c[nH]c3ccccc23)NC(=O)[C@H](CC(=O)O)NC(=O)CCC(=O)NCCCOCCOCCOCCCNC(=O)CN2CCN(CC(=O)O)CCN(CC(=O)O)CCN(CC(=O)O)CC2)CSCc2cccc(c2)CSCCNC1=O]

compound DOTA-Ttds-Nmd-Trp-Trp-Pro-Glu-Leu-[Cys(3MeBn)-Aib-Leu-Ile-AET] (GIP-130, SEQ ID NO: 131) of the following formula

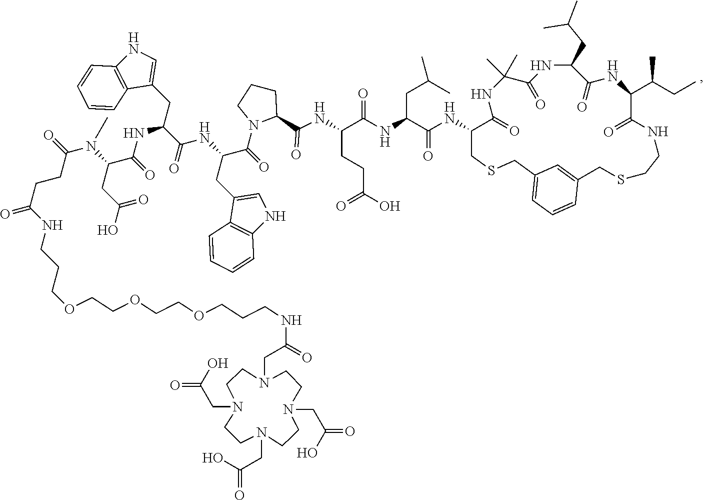 [SMILES: CC[C@H](C)[C@@H]1NC(=O)[C@H](CC(C)C)NC(=O)C(C)(C)NC(=O)[C@@H](NC(=O)[C@H](CC(C)C)NC(=O)[C@H](CCC(=O)O)NC(=O)[C@@H]2CCCN2C(=O)[C@H](Cc2c[nH]c3ccccc23)NC(=O)[C@H](Cc2c[nH]c3ccccc23)NC(=O)[C@H](CC(=O)O)N(C)C(=O)CCC(=O)NCCCOCCOCCOCCCNC(=O)CN2CCN(CC(=O)O)CCN(CC(=O)O)CCN(CC(=O)O)CC2)CSCc2cccc(c2)CSCCNC1=O]

compound Ac-Asp-Trp-Trp-Pro-Glu-Leu-[Cys(3MeBn)-Aib-Leu-Ile-Cys]-Ttds-Lys(DOTA)-NH₂ (GIP-131, SEQ ID NO: 132) of the following formula

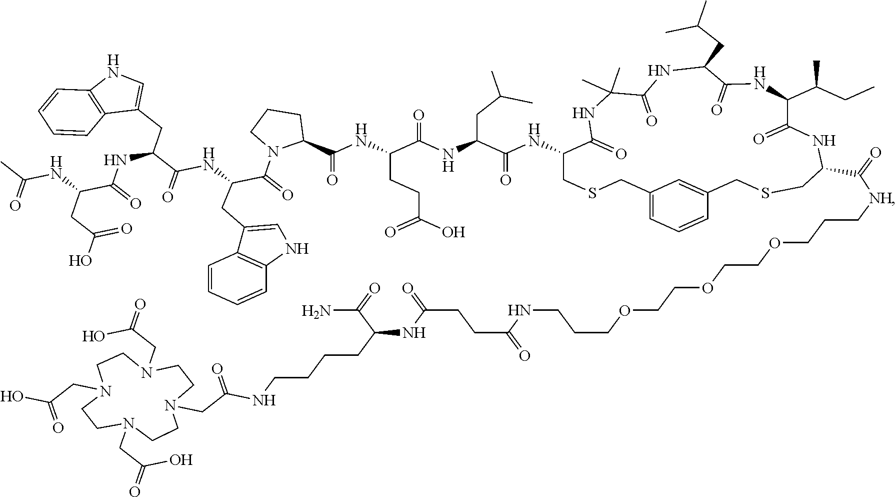 [SMILES: CC[C@H](C)[C@@H]1NC(=O)[C@H](CC(C)C)NC(=O)C(C)(C)NC(=O)[C@@H](NC(=O)[C@H](CC(C)C)NC(=O)[C@H](CCC(=O)O)NC(=O)[C@@H]2CCCN2C(=O)[C@H](Cc2c[nH]c3ccccc23)NC(=O)[C@H](Cc2c[nH]c3ccccc23)NC(=O)[C@H](CC(=O)O)NC(C)=O)CSCc2cccc(c2)CSC[C@@H](C(=O)NCCCOCCOCCOCCCNC(=O)CCC(=O)N[C@@H](CCCCNC(=O)CN2CCN(CC(=O)O)CCN(CC(=O)O)CCN(CC(=O)O)CC2)C(N)=O)NC1=O]

compound Ac-Asp-Trp-Trp-Pro-Glu-Npg-[Cys(3MeBn)-Aib-Leu-Ile-Cys]-Ttds-Lys(DOTA)-NH₂ (GIP-132, SEQ ID NO: 133) of the following formula

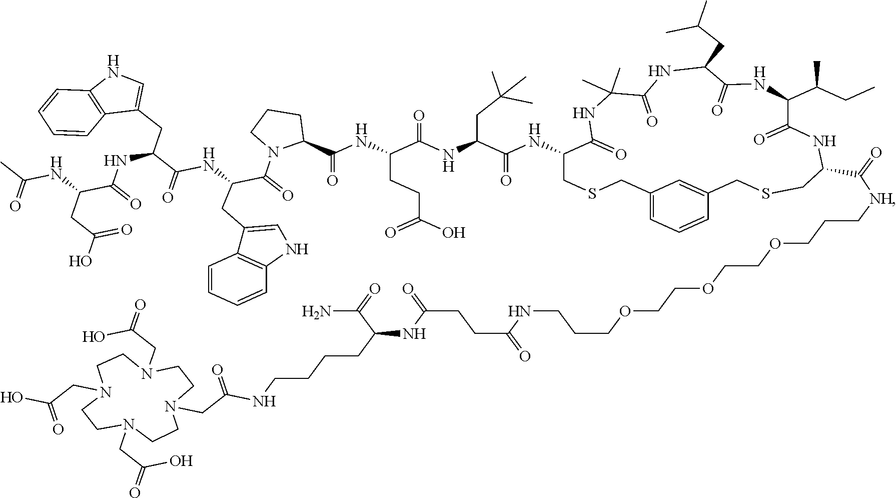 [SMILES: CC[C@H](C)[C@@H]1NC(=O)[C@H](CC(C)C)NC(=O)C(C)(C)NC(=O)[C@@H](NC(=O)[C@H](CC(C)(C)C)NC(=O)[C@H](CCC(=O)O)NC(=O)[C@@H]2CCCN2C(=O)[C@H](Cc2c[nH]c3ccccc23)NC(=O)[C@H](Cc2c[nH]c3ccccc23)NC(=O)[C@H](CC(=O)O)NC(C)=O)CSCc2cccc(c2)CSC[C@@H](C(=O)NCCCOCCOCCOCCCNC(=O)CCC(=O)N[C@@H](CCCCNC(=O)CN2CCN(CC(=O)O)CCN(CC(=O)O)CCN(CC(=O)O)CC2)C(N)=O)NC1=O]

compound Ac-Asp-Trp-Trp-Pro-Glu-Leu-[Cys(3MeBn)-Aib-Npg-Ile-Cys]-Ttds-Lys(DOTA)-NH₂ (GIP-133, SEQ ID NO: 134) of the following formula

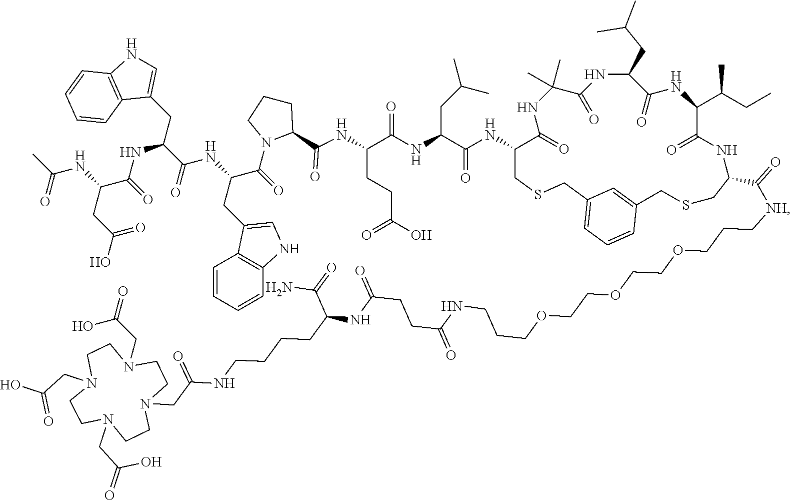 [SMILES: CC[C@H](C)[C@@H]1NC(=O)[C@H](CC(C)C)NC(=O)C(C)(C)NC(=O)[C@@H](NC(=O)[C@H](CC(C)C)NC(=O)[C@H](CCC(=O)O)NC(=O)[C@@H]2CCCN2C(=O)[C@H](Cc2c[nH]c3ccccc23)NC(=O)[C@H](Cc2c[nH]c3ccccc23)NC(=O)[C@H](CC(=O)O)NC(C)=O)CSCc2cccc(c2)CSC[C@@H](C(=O)NCCCOCCOCCOCCCNC(=O)CCC(=O)N[C@@H](CCCCNC(=O)CN2CCN(CC(=O)O)CCN(CC(=O)O)CCN(CC(=O)O)CC2)C(N)=O)NC1=O]

compound Ac-Asp-Trp-Trp-Pro-Glu-Leu-[Cys(3MeBn)-Aib-Cha-Ile-Cys]-Ttds-Lys(DOTA)-NH₂ (GIP-134, SEQ ID NO: 135) of the following formula

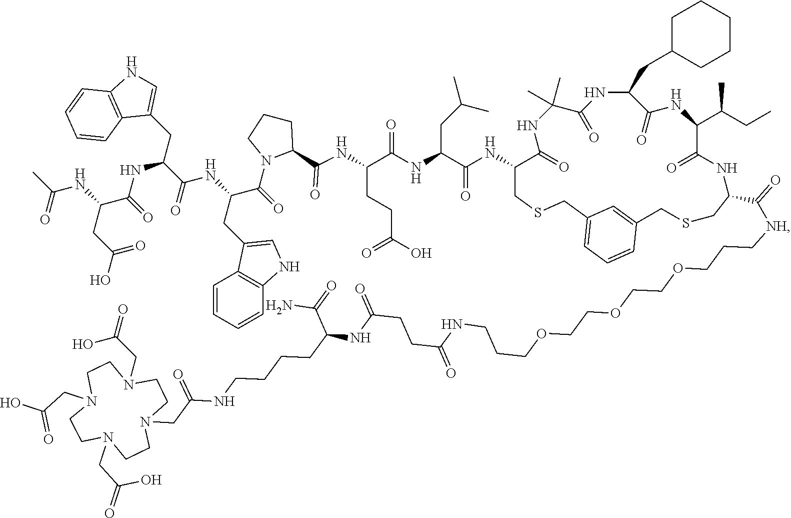 [SMILES: CC[C@H](C)[C@@H]1NC(=O)[C@H](CC2CCCCC2)NC(=O)C(C)(C)NC(=O)[C@@H](NC(=O)[C@H](CC(C)C)NC(=O)[C@H](CCC(=O)O)NC(=O)[C@@H]2CCCN2C(=O)[C@H](Cc2c[nH]c3ccccc23)NC(=O)[C@H](Cc2c[nH]c3ccccc23)NC(=O)[C@H](CC(=O)O)NC(C)=O)CSCc2cccc(c2)CSC[C@@H](C(=O)NCCCOCCOCCOCCCNC(=O)CCC(=O)N[C@@H](CCCCNC(=O)CN2CCN(CC(=O)O)CCN(CC(=O)O)CCN(CC(=O)O)CC2)C(N)=O)NC1=O]

compound Php-Asp-Trp-Trp-Pro-Glu-leu-[Cys(3MeBn)-Aib-Leu-Ile-Cys]-Ttds-Lys(DOTA)-NH₂ (GIP-135, SEQ ID NO: 136) of the following formula

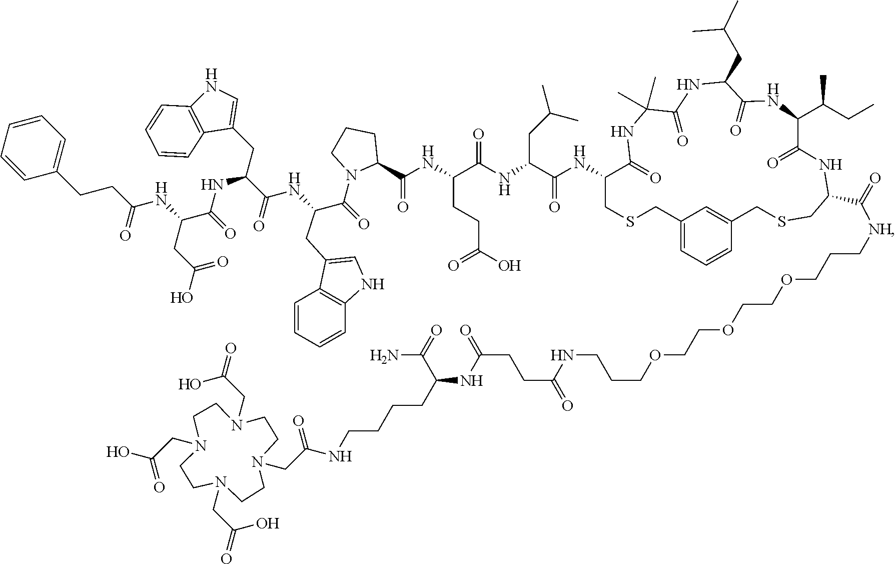 [SMILES: CC[C@H](C)[C@@H]1NC(=O)[C@H](CC(C)C)NC(=O)C(C)(C)NC(=O)[C@@H](NC(=O)[C@@H](CC(C)C)NC(=O)[C@H](CCC(=O)O)NC(=O)[C@@H]2CCCN2C(=O)[C@H](Cc2c[nH]c3ccccc23)NC(=O)[C@H](Cc2c[nH]c3ccccc23)NC(=O)[C@H](CC(=O)O)NC(=O)CCc2ccccc2)CSCc2cccc(c2)CSC[C@@H](C(=O)NCCCOCCOCCOCCCNC(=O)CCC(=O)N[C@@H](CCCCNC(=O)CN2CCN(CC(=O)O)CCN(CC(=O)O)CCN(CC(=O)O)CC2)C(N)=O)NC1=O]

compound 4FPhp-Asp-Trp-Trp-Pro-Glu-leu-[Cys(3MeBn)-Aib-Leu-Ile-Cys]-Ttds-Lys(DOTA)-NH₂ (GIP-136, SEQ ID NO: 137) of the following formula

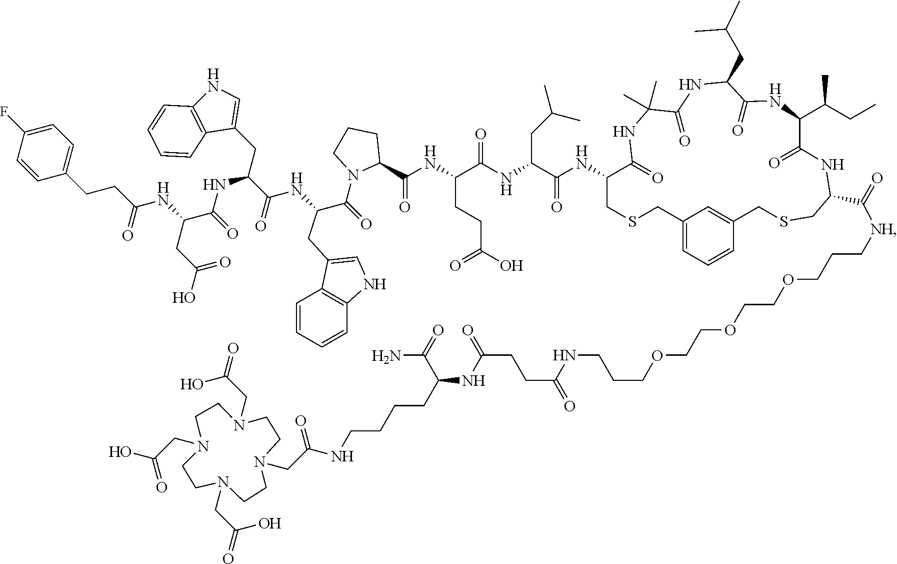 [SMILES: CC[C@H](C)[C@@H]1NC(=O)[C@H](CC(C)C)NC(=O)C(C)(C)NC(=O)[C@@H](NC(=O)[C@@H](CC(C)C)NC(=O)[C@H](CCC(=O)O)NC(=O)[C@@H]2CCCN2C(=O)[C@H](Cc2c[nH]c3ccccc23)NC(=O)[C@H](Cc2c[nH]c3ccccc23)NC(=O)[C@H](CC(=O)O)NC(=O)CCc2ccc(F)cc2)CSCc2cccc(c2)CSC[C@@H](C(=O)NCCCOCCOCCOCCCNC(=O)CCC(=O)N[C@@H](CCCCNC(=O)CN2CCN(CC(=O)O)CCN(CC(=O)O)CCN(CC(=O)O)CC2)C(N)=O)NC1=O]

compound 4OHPhp-Asp-Trp-Trp-Pro-Glu-leu-[Cys(3MeBn)-Aib-Leu-Ile-Cys]-Ttds-Lys(DOTA)-NH₂ (GIP-137, SEQ ID NO: 138) of the following formula

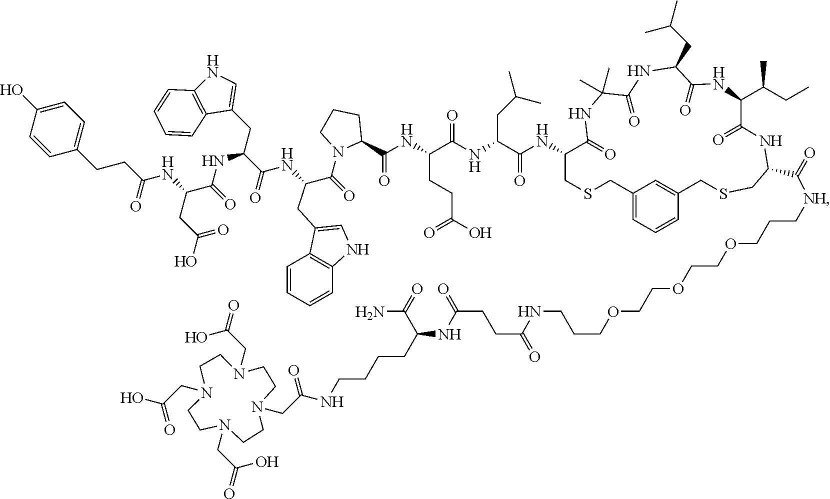 [SMILES: CC[C@H](C)[C@@H]1NC(=O)[C@H](CC(C)C)NC(=O)C(C)(C)NC(=O)[C@@H](NC(=O)[C@@H](CC(C)C)NC(=O)[C@H](CCC(=O)O)NC(=O)[C@@H]2CCCN2C(=O)[C@H](Cc2c[nH]c3ccccc23)NC(=O)[C@H](Cc2c[nH]c3ccccc23)NC(=O)[C@H](CC(=O)O)NC(=O)CCc2ccc(O)cc2)CSCc2cccc(c2)CSC[C@@H](C(=O)NCCCOCCOCCOCCCNC(=O)CCC(=O)N[C@@H](CCCCNC(=O)CN2CCN(CC(=O)O)CCN(CC(=O)O)CCN(CC(=O)O)CC2)C(N)=O)NC1=O]

compound Ac-Asp-Trp-1Ni-Pro-Glu-leu-[Cys(3MeBn)-Ala-Leu-Val-Cys]-Ttds-Lys(DOTA)-NH₂ (GIP-138, SEQ ID NO: 139) of the following formula

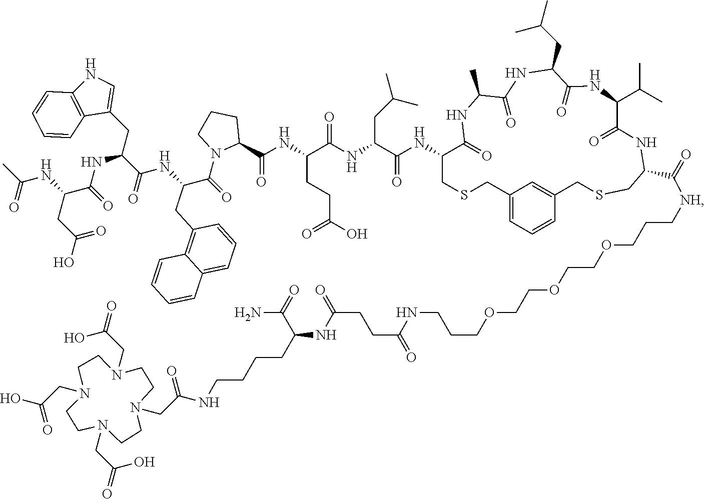 [SMILES: CC(=O)N[C@@H](CC(=O)O)C(=O)N[C@@H](Cc1c[nH]c2ccccc12)C(=O)N[C@@H](Cc1cccc2ccccc12)C(=O)N1CCC[C@H]1C(=O)N[C@@H](CCC(=O)O)C(=O)N[C@H](CC(C)C)C(=O)N[C@H]1CSCc2cccc(c2)CSC[C@@H](C(=O)NCCCOCCOCCOCCCNC(=O)CCC(=O)N[C@@H](CCCCNC(=O)CN2CCN(CC(=O)O)CCN(CC(=O)O)CCN(CC(=O)O)CC2)C(N)=O)NC(=O)[C@H](C(C)C)NC(=O)[C@H](CC(C)C)NC(=O)[C@H](C)NC1=O]

compound Ac-Asp-Trp-1Ni-Pro-Glu-leu-[Cys(3MeBn)-Deg-Leu-Ile-Cys]-Ttds-Lys(DOTA)-NH₂ (GIP-139, SEQ ID NO: 140) of the following formula

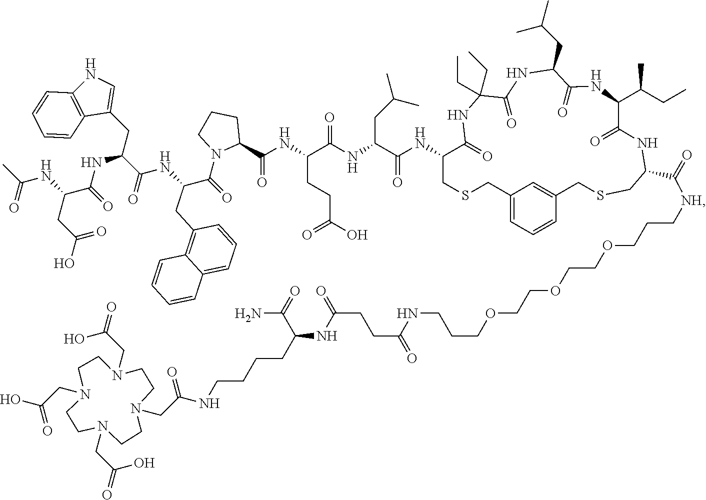 [SMILES: CC[C@H](C)[C@@H]1NC(=O)[C@H](CC(C)C)NC(=O)C(CC)(CC)NC(=O)[C@@H](NC(=O)[C@@H](CC(C)C)NC(=O)[C@H](CCC(=O)O)NC(=O)[C@@H]2CCCN2C(=O)[C@H](Cc2cccc3ccccc23)NC(=O)[C@H](Cc2c[nH]c3ccccc23)NC(=O)[C@H](CC(=O)O)NC(C)=O)CSCc2cccc(c2)CSC[C@@H](C(=O)NCCCOCCOCCOCCCNC(=O)CCC(=O)N[C@@H](CCCCNC(=O)CN2CCN(CC(=O)O)CCN(CC(=O)O)CCN(CC(=O)O)CC2)C(N)=O)NC1=O]

compound Ac-Asp-Trp-6Fw-Pro-Glu-leu-[Cys(3MeBn)-Aib-Leu-Ile-Cys]-Ttds-Lys(DOTA)-NH₂ (GIP-140, SEQ ID NO: 141) of the following formula

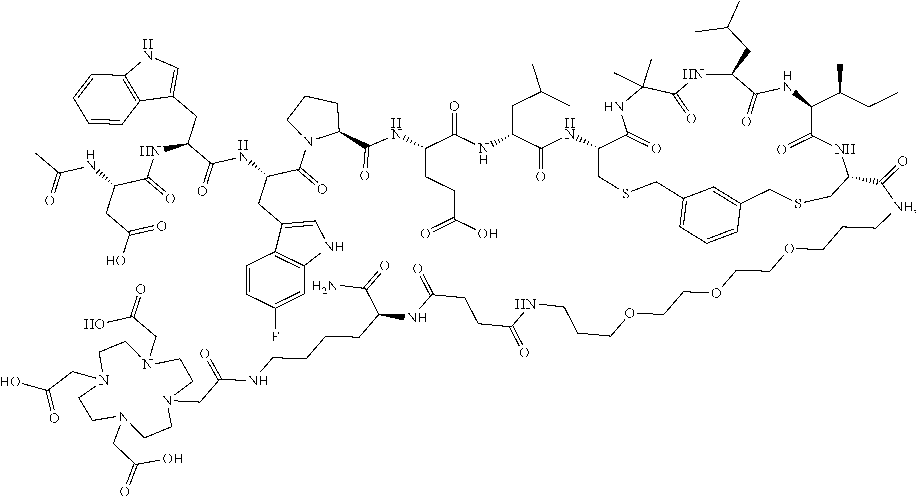 [SMILES: CC[C@H](C)[C@@H]1NC(=O)[C@H](CC(C)C)NC(=O)C(C)(C)NC(=O)[C@@H](NC(=O)[C@@H](CC(C)C)NC(=O)[C@H](CCC(=O)O)NC(=O)[C@@H]2CCCN2C(=O)[C@H](Cc2c[nH]c3cc(F)ccc23)NC(=O)[C@H](Cc2c[nH]c3ccccc23)NC(=O)[C@H](CC(=O)O)NC(C)=O)CSCc2cccc(c2)CSC[C@@H](C(=O)NCCCOCCOCCOCCCNC(=O)CCC(=O)N[C@@H](CCCCNC(=O)CN2CCN(CC(=O)O)CCN(CC(=O)O)CCN(CC(=O)O)CC2)C(N)=O)NC1=O]

compound DOTA-Ttds-Asp-6Clw-Trp-Pro-Glu-leu-[Cys(3MeBn)-Aib-Leu-Ile-AET] (GIP-141, SEQ ID NO: 142) of the following formula

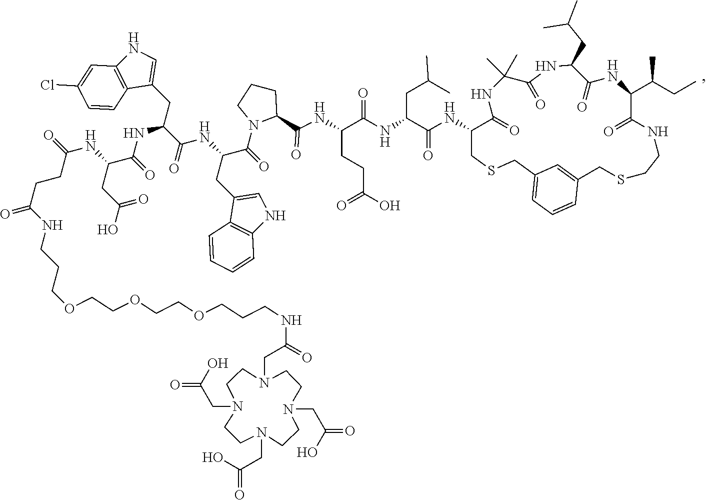 [SMILES: CC[C@H](C)[C@@H]1NC(=O)[C@H](CC(C)C)NC(=O)C(C)(C)NC(=O)[C@@H](NC(=O)[C@@H](CC(C)C)NC(=O)[C@H](CCC(=O)O)NC(=O)[C@@H]2CCCN2C(=O)[C@H](Cc2c[nH]c3ccccc23)NC(=O)[C@H](Cc2c[nH]c3cc(Cl)ccc23)NC(=O)[C@H](CC(=O)O)NC(=O)CCC(=O)NCCCOCCOCCOCCCNC(=O)CN2CCN(CC(=O)O)CCN(CC(=O)O)CCN(CC(=O)O)CC2)CSCc2cccc(c2)CSCCNC1=O]

compound HO-Succinyl-Trp-5Clw-Hyp-Glu-leu-[Cys(2Lut)-Apc(MeO10kDaPEGAc)-Leu-Ile-Cys]-APAc-Lys(DOTA)-NH₂ (GIP-142, SEQ ID NO: 143) of the following formula

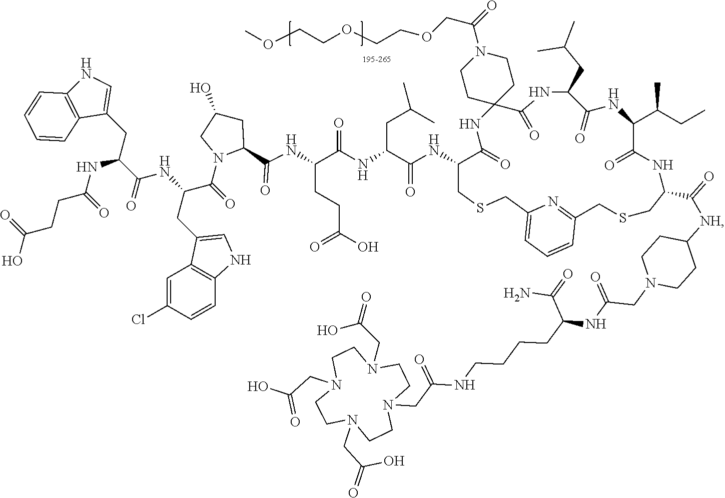

compound HO-Succinyl-Trp-5Clw-Hyp-Glu-leu-[Cys(2Lut)-Apc(MeO2kDaPEGAc)-Leu-Ile-Cys]-APAc-Lys(DOTA)-NH₂ (GIP-143, SEQ ID NO: 144) of the following formula

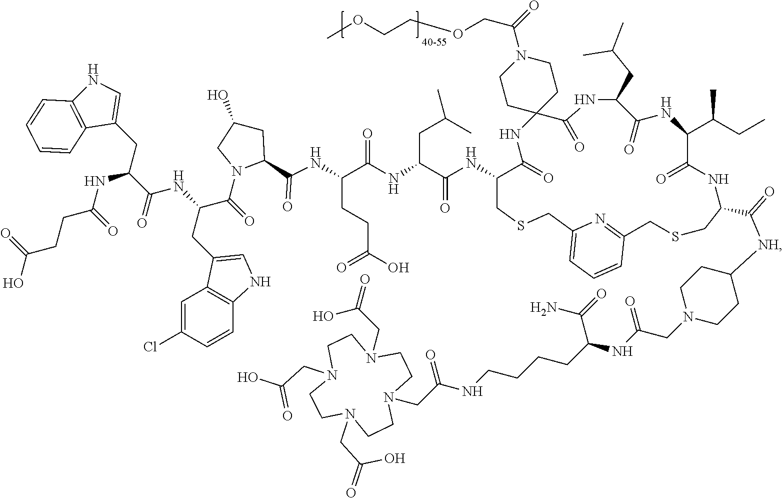

compound HO-Succinyl-Trp-5Clw-Hyp-Glu-leu-[Cys(2Lut)-Thp-Leu-Ile-Cys]-APAc-lys(MeO2kDaPEGAc)-Lys(DOTA)-NH₂ (GIP-144, SEQ ID NO: 145) of the following formula

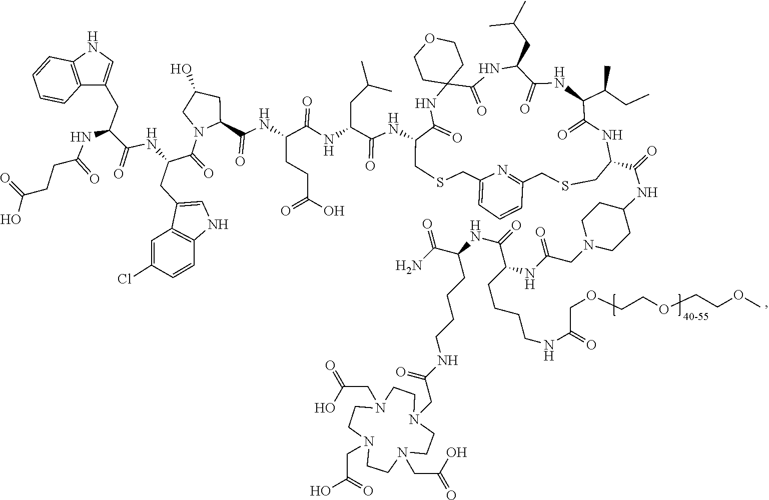

compound Ac-Asp-Trp-5Clw-Hyp-Glu-leu-[Cys(tMeBn(DOTA-PP))-Thp-Leu-Ile-Cys]-NH₂ (GIP-145, SEQ ID NO: 146) of the following formula

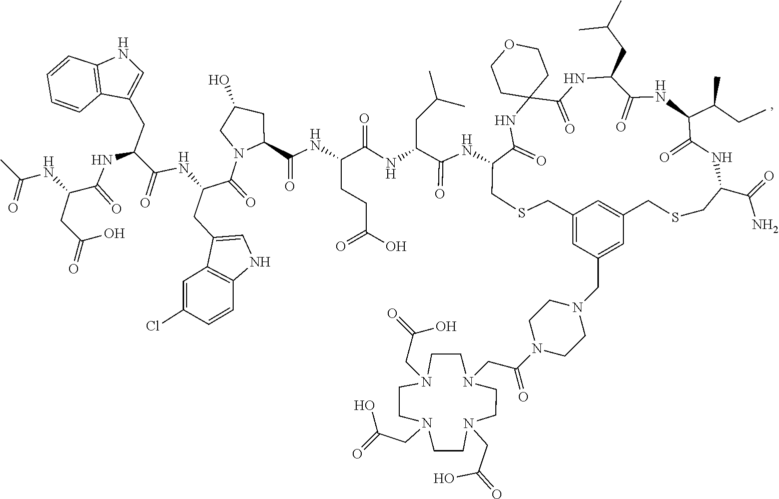 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CC(=O)O)NC(C)=O)CSCc2cc(cc(CN3CCN(C(=O)CN4CCN(CC(=O)O)CCN(CC(=O)O)CCN(CC(=O)O)CC4)CC3)c2)CSC[C@@H](C(N)=O)NC1=O]

compound AGLU-Glutar-Asp-Trp-5Clw-Hyp-Glu-leu-[Cys(2Lut)-Apc(InDOTA)-Leu-Ile-AET] (GIP-146, SEQ ID NO: 147) of the following formula

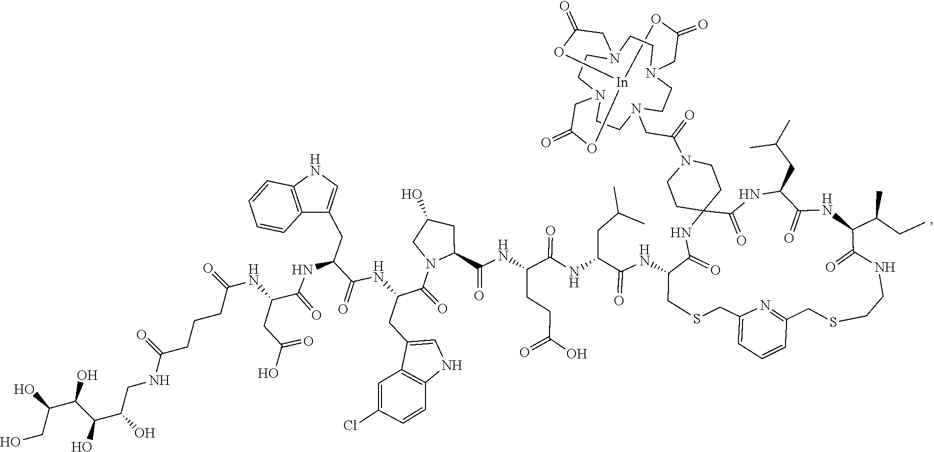

compound AGLU-Glutar-Asp-Trp-5Clw-Hyp-Glu-leu-[Cys(2Lut)-Apc(ZnDOTA)-Leu-Ile-AET] (GIP-147, SEQ ID NO: 148) of the following formula

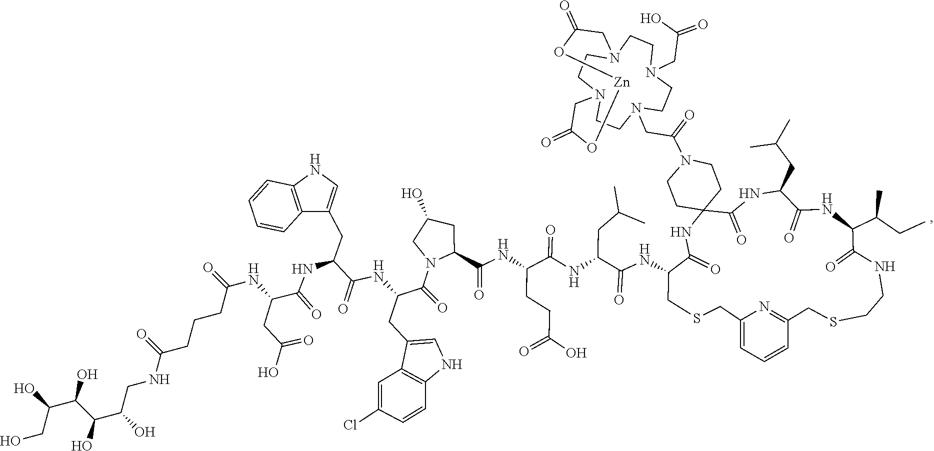

compound InDOTA-Ttds-Asp-Trp-5Clw-Pro-Glu-leu-[Cys(3MeBn)-Aib-Leu-Ile-AET] (GIP-148, SEQ ID NO: 149) of the following formula

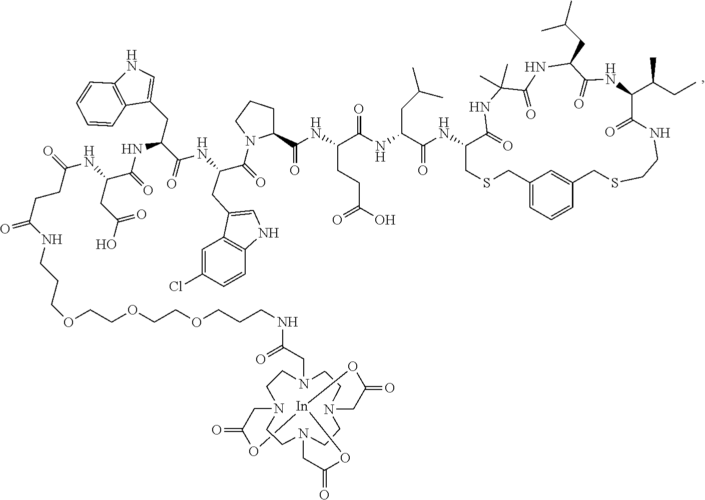

compound InDOTA-Ttds-Asp-Trp-5Clw-Pro-Glu-leu-[Cys(3MeBn)-Ams-Leu-Ile-AET] (GIP-149, SEQ ID NO: 150) of the following formula

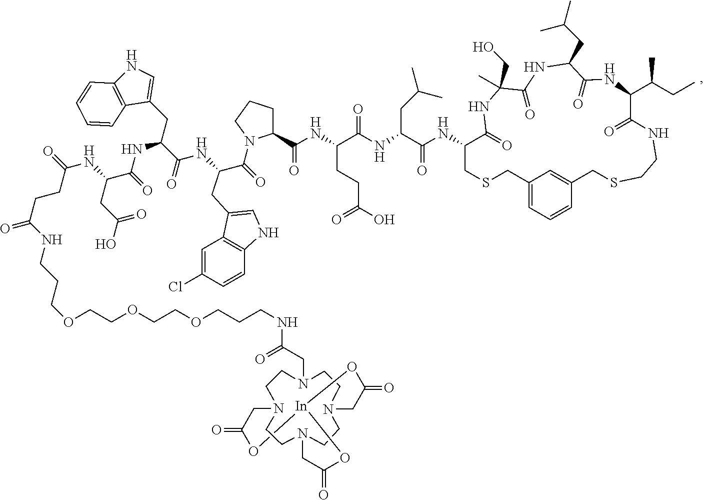

compound InDOTA-Ttds-Asp-Trp-5Clw-Pro-Glu-leu-[Cys(3MeBn)-Thp-Leu-Ile-AET] (GIP-150, SEQ ID NO: 151) of the following formula

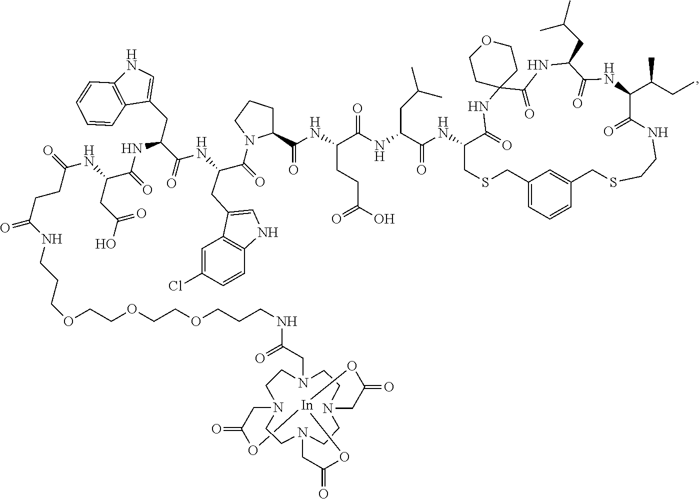

compound InDOTA-Ttds-Asp-Trp-5Clw-Hyp-Glu-leu-[Cys(3MeBn)-Aib-Leu-Ile-AET] (GIP-151, SEQ ID NO: 152) of the following formula

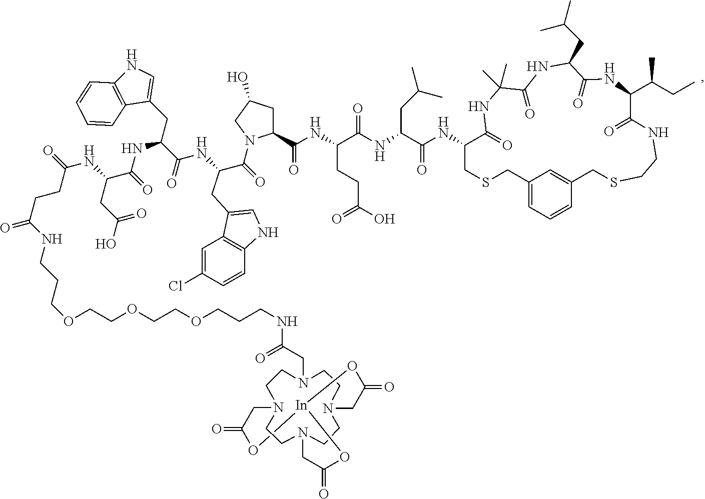

compound InDOTA-Ttds-Asp-Trp-5Clw-Nmg-Glu-leu-[Cys(3MeBn)-Aib-Leu-Ile-AET] (GIP-152, SEQ ID NO: 153) of the following formula

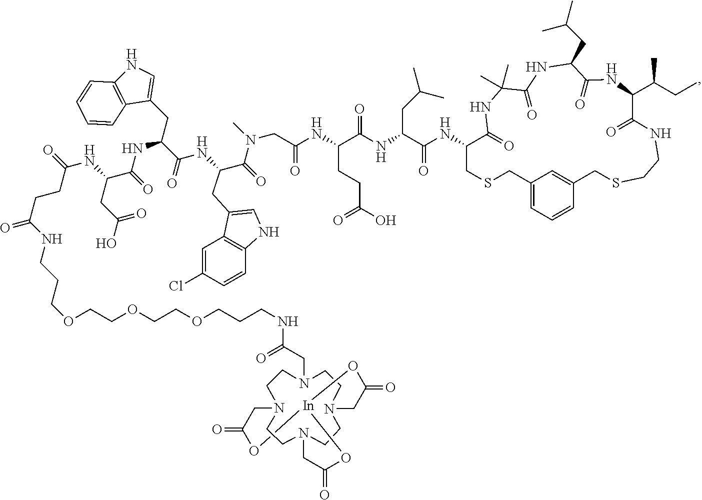

compound InDOTA-APAc-Asp-Trp-5Clw-Pro-Glu-leu-[Cys(3MeBn)-Aib-Leu-Ile-AET] (GIP-153, SEQ ID NO: 154) of the following formula

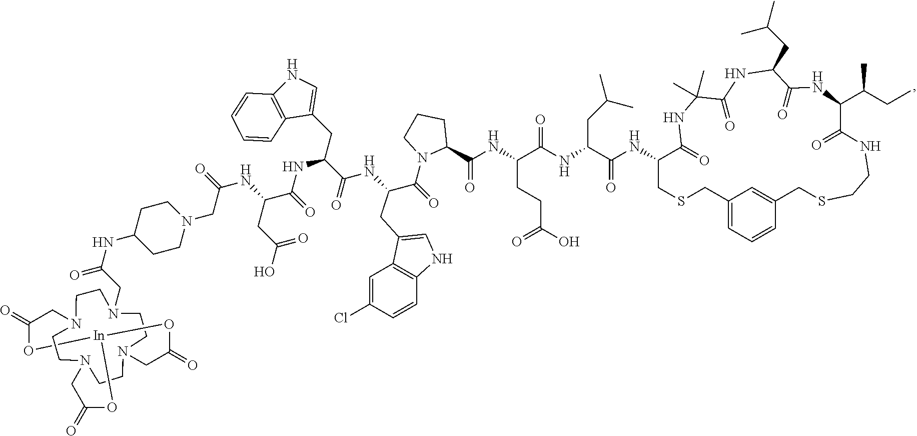

compound InDOTA-Ttds-Asp-Trp-5Clw-Pro-Glu-leu-[Cys(2Lut)-Aib-Leu-Ile-AET] (GIP-154, SEQ ID NO: 155) of the following formula

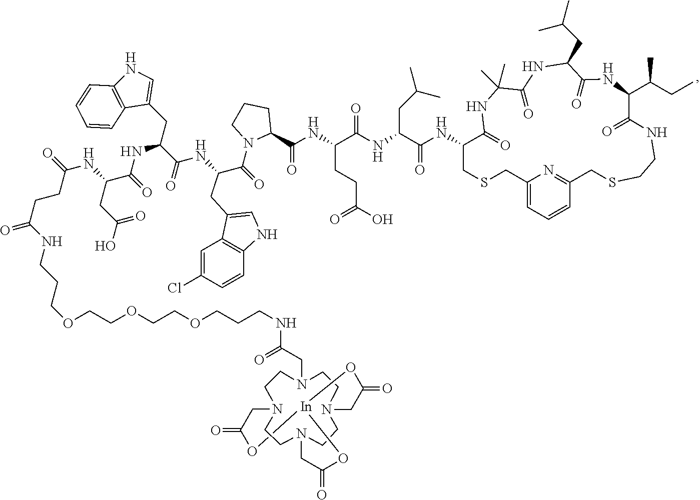

compound InDOTA-APAc-Asp-Trp-5Clw-Hyp-Glu-leu-[Cys(2Lut)-Thp-Leu-Ile-AET] (GIP-155, SEQ ID NO: 156) of the following formula

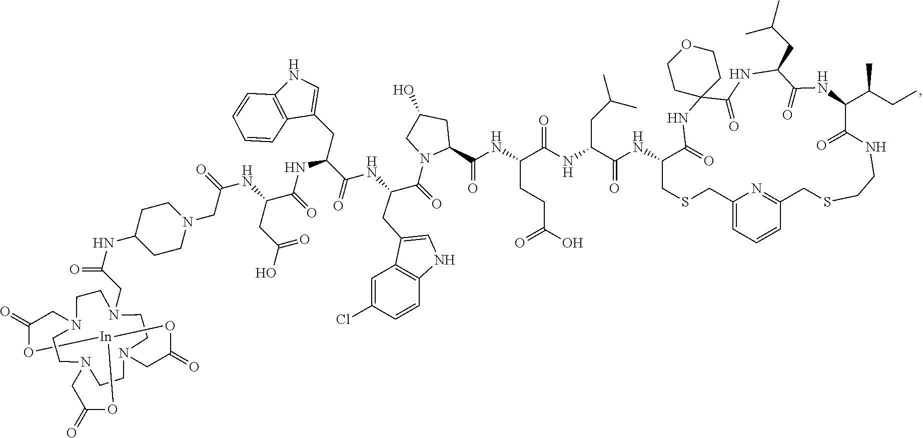

compound Ac-Asp-Trp-5Clw-Pro-Glu-leu-[Cys(3MeBn)-Aib-Leu-Ile-Cys]-Ttds-Lys(LuDOTA)-NH₂ (GIP-156, SEQ ID NO: 157) of the following formula

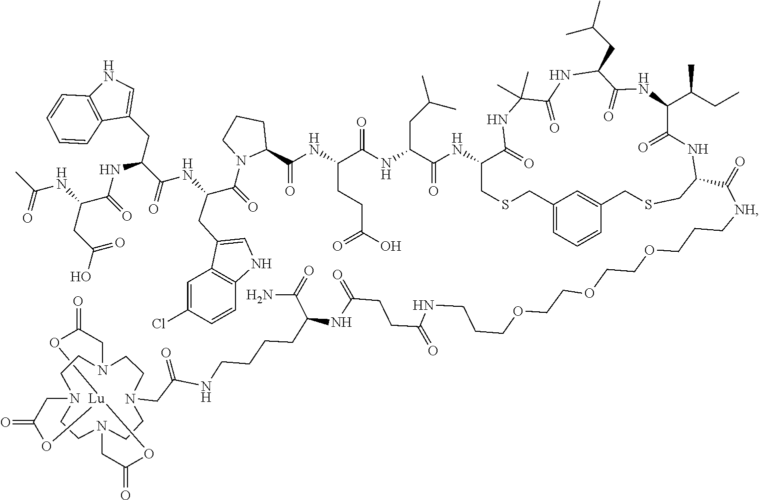

compound LuDOTA-Ttds-Asp-Trp-5Clw-Pro-Glu-leu-[Cys(3MeBn)-Ams-Leu-Ile-AET] (GIP-157, SEQ ID NO: 158) of the following formula

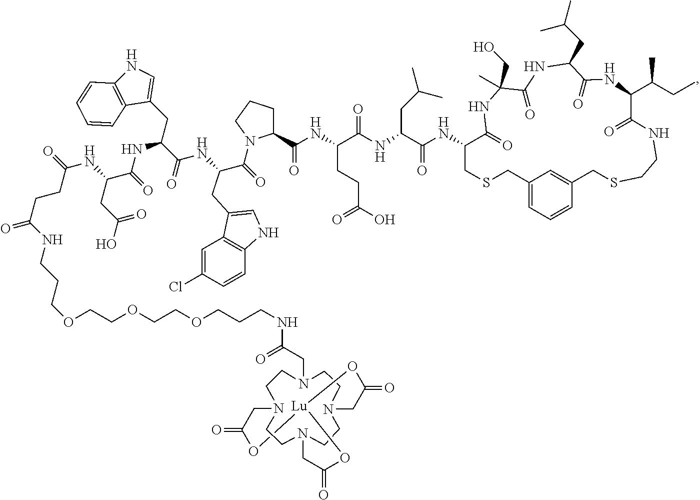

compound LuDOTA-Ttds-Asp-Trp-5Clw-Pro-Glu-leu-[Cys(3MeBn)-Thp-Leu-Ile-AET] (GIP-158, SEQ ID NO: 159) of the following formula

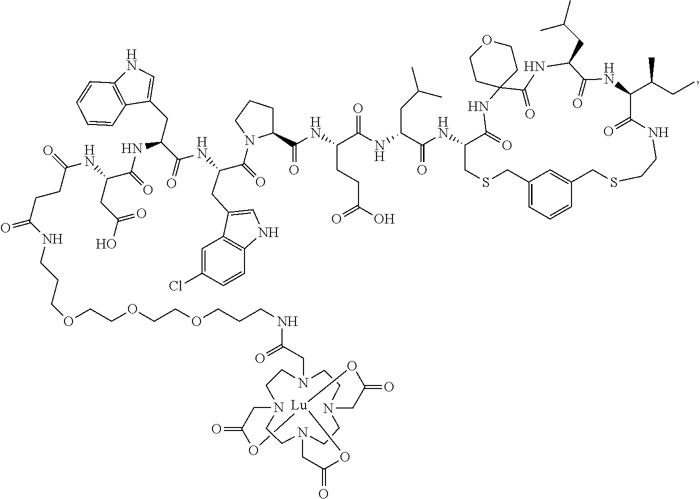

compound LuDOTA-Ttds-Asp-Trp-5Clw-Hyp-Glu-leu-[Cys(3MeBn)-Aib-Leu-Ile-AET] (GIP-159, SEQ ID NO: 160) of the following formula

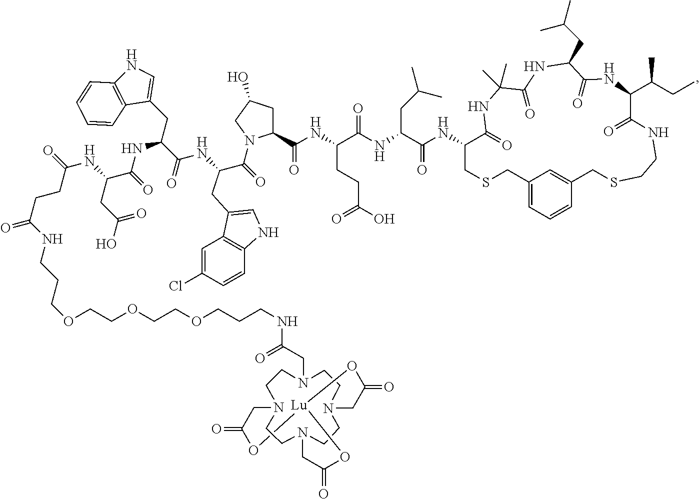

compound LuDOTA-Ttds-Asp-Trp-5Clw-Nmg-Glu-leu-[Cys(3MeBn)-Aib-Leu-Ile-AET] (GIP-160, SEQ ID NO: 161) of the following formula

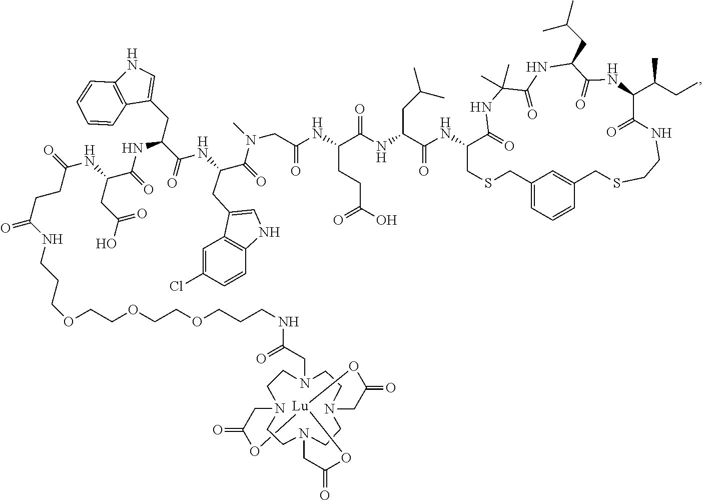

compound LuDOTA-APAc-Asp-Trp-5Clw-Pro-Glu-leu-[Cys(3MeBn)-Aib-Leu-Ile-AET] (GIP-161, SEQ ID NO: 162) of the following formula

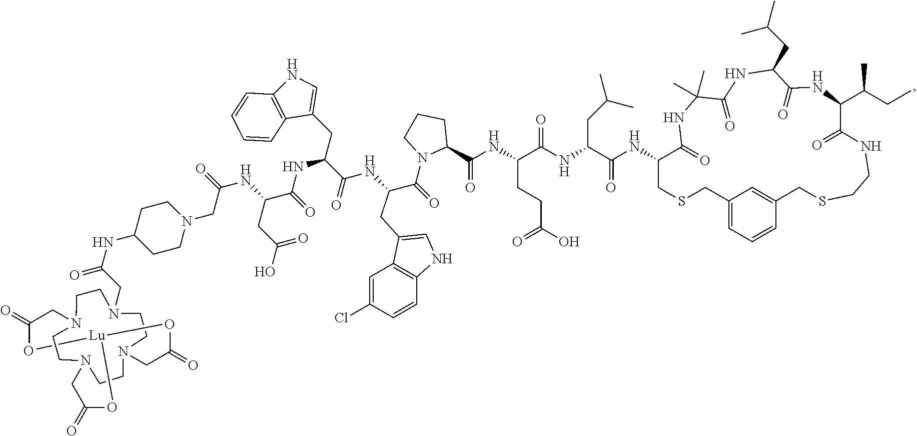

compound LuDOTA-APAc-Asp-Trp-5Clw-Hyp-Glu-leu-[Cys(2Lut)-Thp-Leu-Ile-AET] (GIP-162, SEQ ID NO: 163) of the following formula

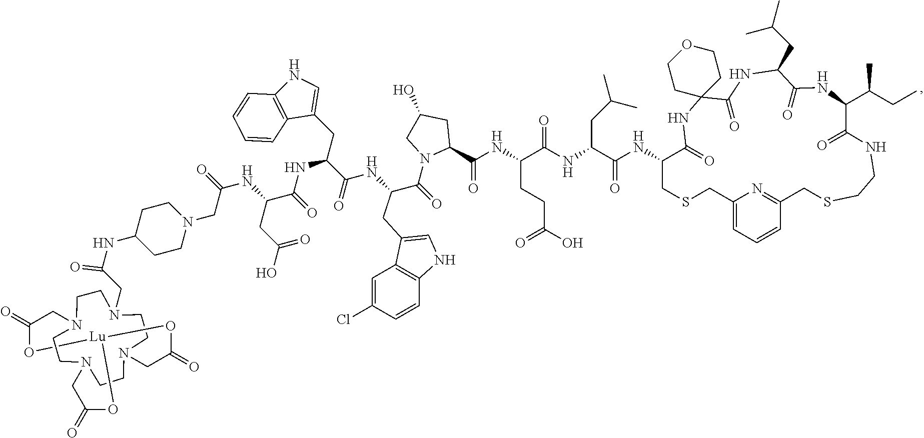

compound LuDOTA-Ttds-Asp-Trp-5Clw-Pro-Glu-leu-[Cys(2Lut)-Aib-Leu-Ile-AET] (GIP-163, SEQ ID NO: 164) of the following formula

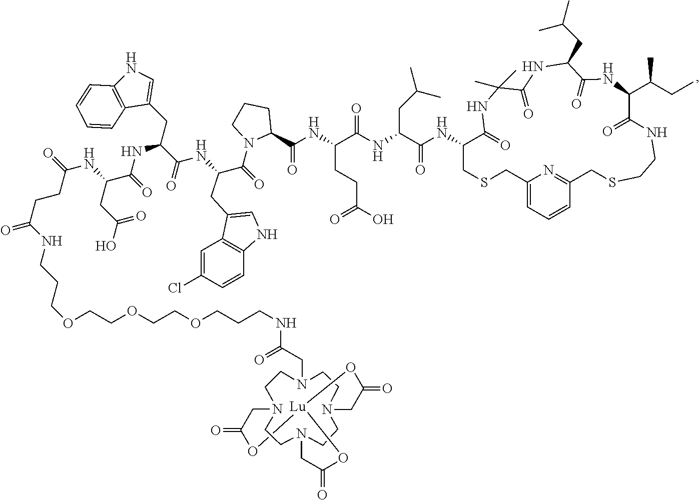

compound Ac-Asp-Trp-5Clw-Pro-Glu-leu-[Cys(3MeBn)-Aib-Leu-Ile-Cys]-Ttds-Lys(InDOTA)-NH₂ (GIP-164, SEQ ID NO: 165) of the following formula

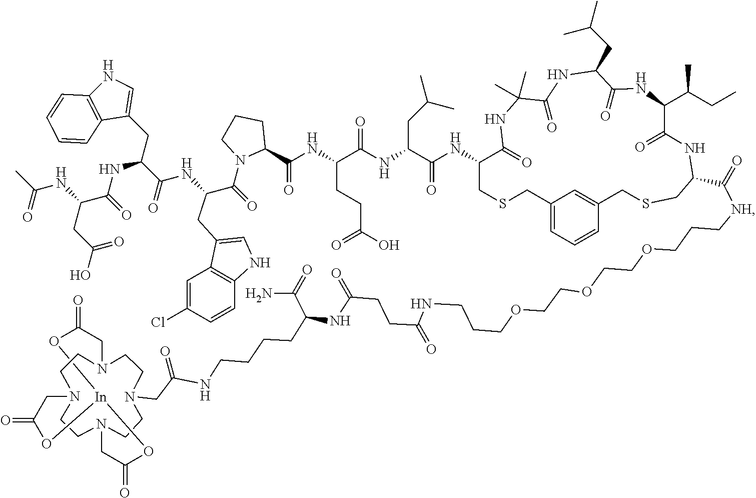

compound Ac-Asp-Trp-1Ni-Pro-Glu-leu-[Cys(3MeBn)-Aib-Leu-Ile-Cys]-Ttds-Lys(InDOTA)-NH₂ (GIP-165, SEQ ID NO: 166) of the following formula

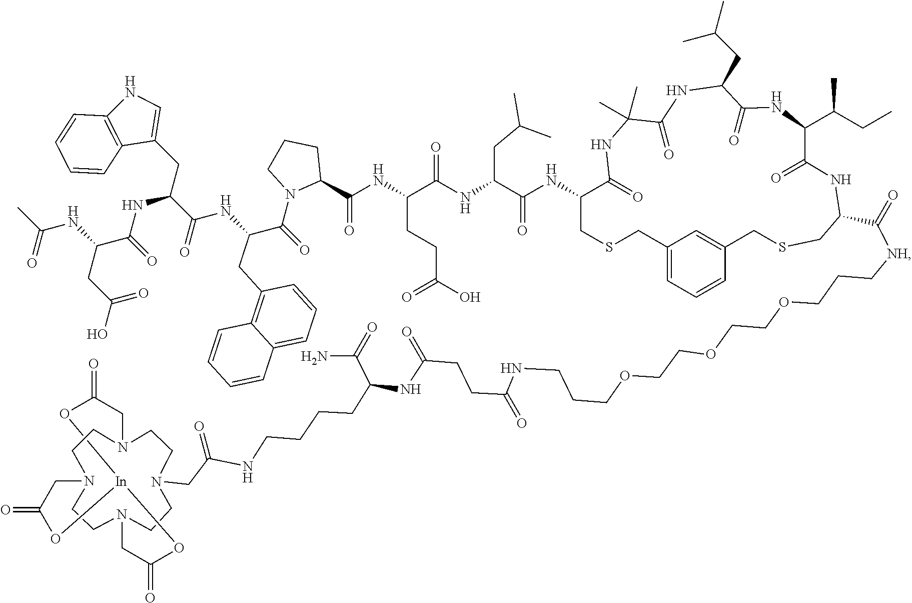

compound Ac-Asp-Trp-1Ni-Pro-Glu-leu-[Cys(3MeBn)-Egz-Leu-Ile-Cys]-Ttds-Lys(InDOTA)-NH₂ (GIP-166, SEQ ID NO: 167) of the following formula

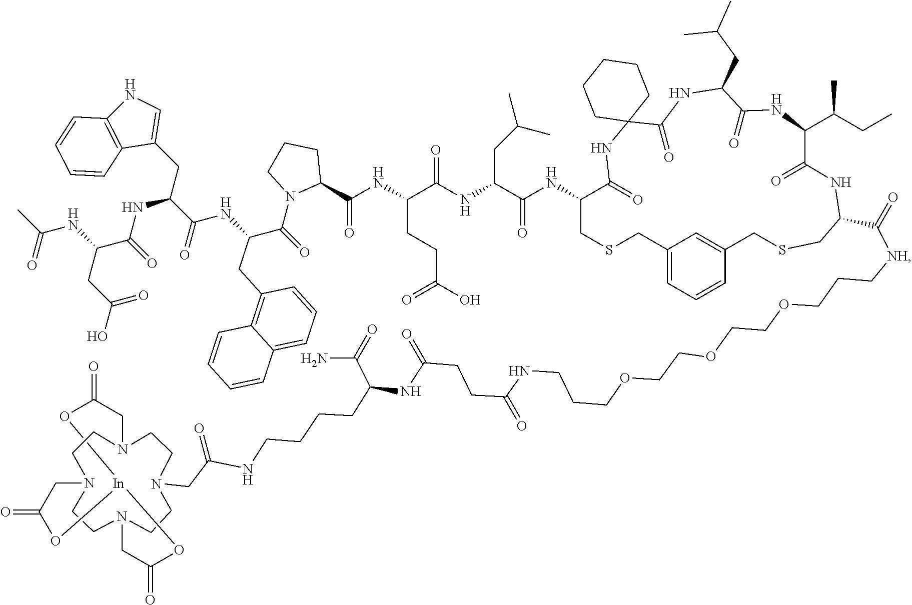

compound LuDOTA-Ttds-Asp-Trp-5Clw-Pro-Glu-leu-[Cys(3MeBn)-Aib-Leu-Ile-AET] (GIP-167, SEQ ID NO: 168) of the following formula

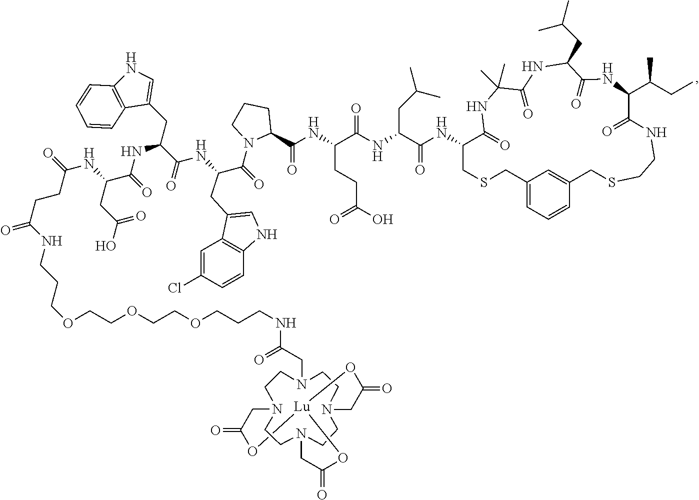

compound HO-Succinyl-Trp-5Clw-Hyp-Glu-leu-[Cys(2Lut)-Thp-Leu-Ile-Cys]-APAc-Lys(InDOTA)-NH₂ (GIP-168, SEQ ID NO: 169) of the following formula

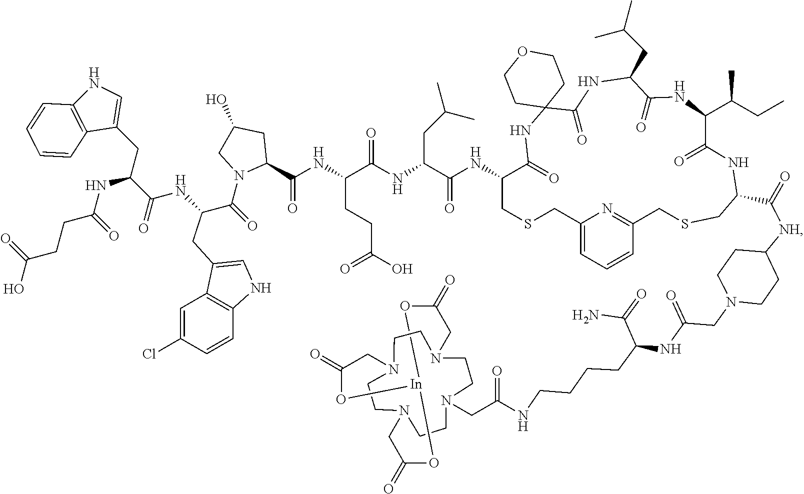

compound HO-Succinyl-Trp-5Clw-Hyp-Glu-leu-[Cys(2Lut)-Apc(InDOTA)-Leu-Ile-AET] (GIP-169, SEQ ID NO: 170) of the following formula

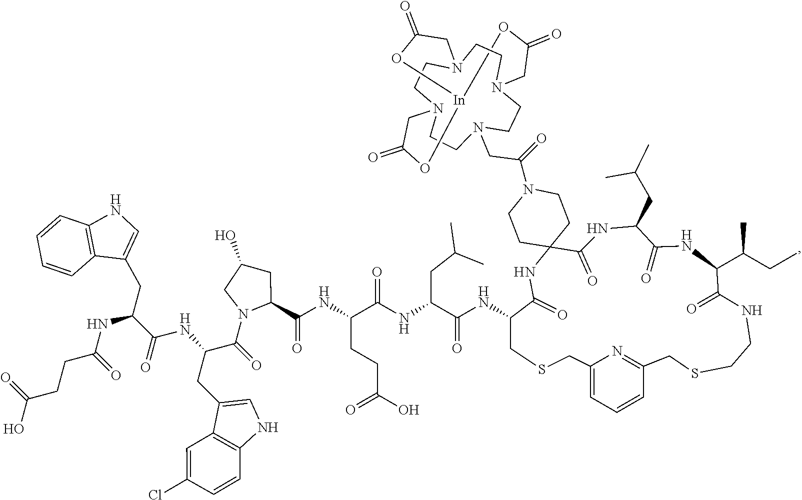

compound HO-Succinyl-Trp-5Clw-Hyp-Glu-leu-[Cys(2Lut)-Thp-Leu-Ile-Cys]-APAc-Lys(LuDOTA)-NH₂ (GIP-170, SEQ ID NO: 171) of the following formula

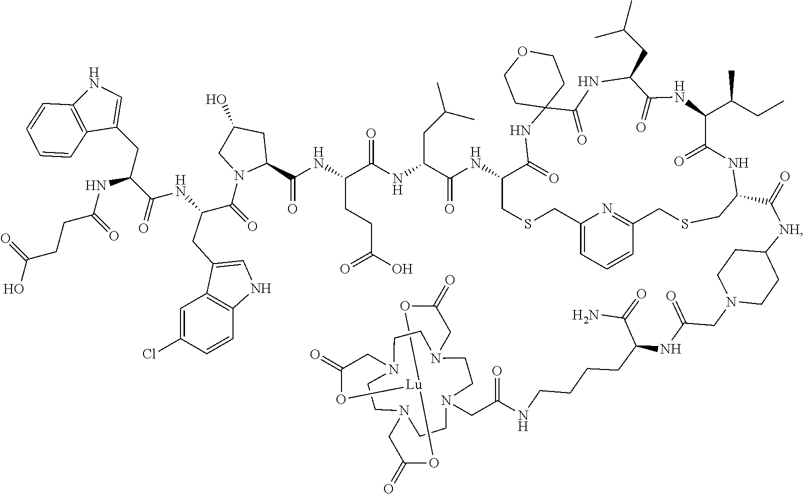

compound HO-Succinyl-Trp-5Clw-Hyp-Glu-leu-[Cys(2Lut)-Apc(LuDOTA)-Leu-Ile-AET] (GIP-171, SEQ ID NO: 172) of the following formula

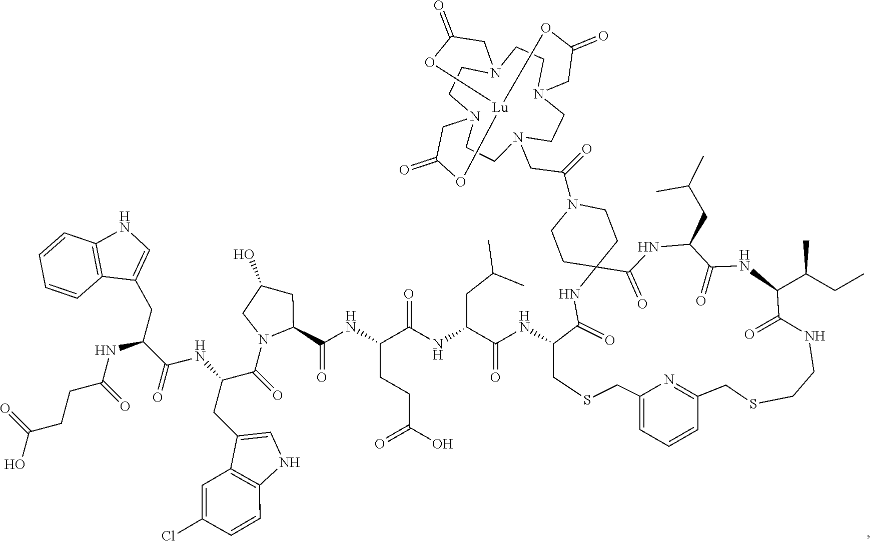

compound Ac-Asp-Trp-1Ni-Pro-Glu-leu-[Cys(3MeBn)-Aib-Leu-Ile-Cys]-Ttds-Lys(LuDOTA)-NH₂ (GIP-172, SEQ ID NO: 173) of the following formula

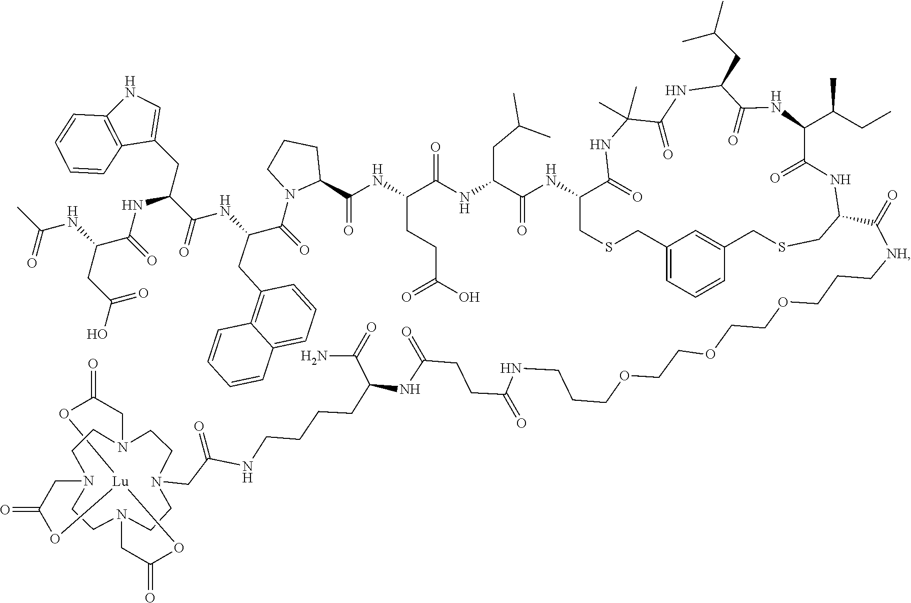

compound Ac-Asp-Trp-1Ni-Pro-Glu-leu-[Cys(3MeBn)-Egz-Leu-Ile-Cys]-Ttds-Lys(LuDOTA)-NH₂ (GIP-173, SEQ ID NO: 174) of the following formula

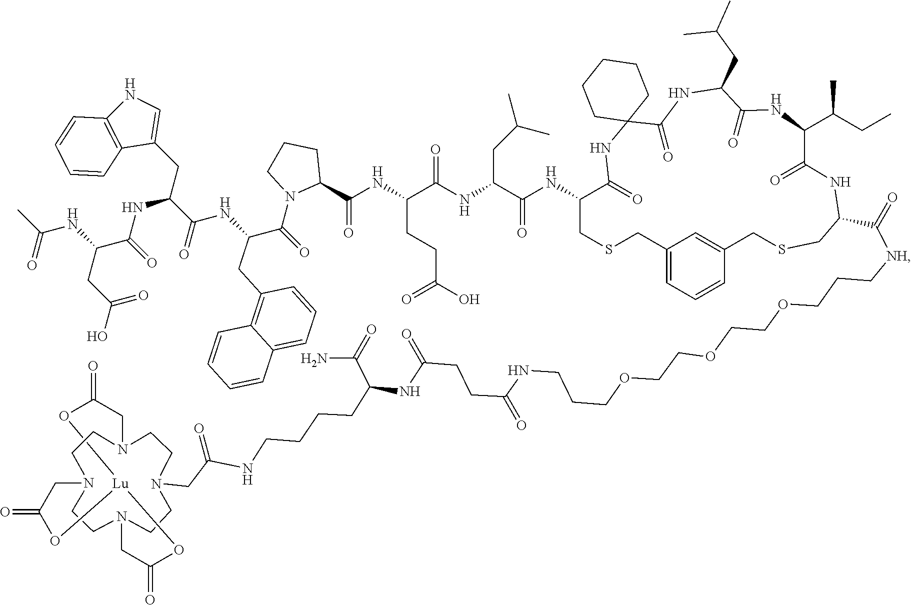

compound AGLU-Glutar-Asp-Trp-5Clw-Hyp-Glu-leu-[Cys(2Lut)-Thp-Leu-Ile-Cys]-APAc-glu(AGLU)-NH₂ (GIP-174, SEQ ID NO: 175) of the following formula

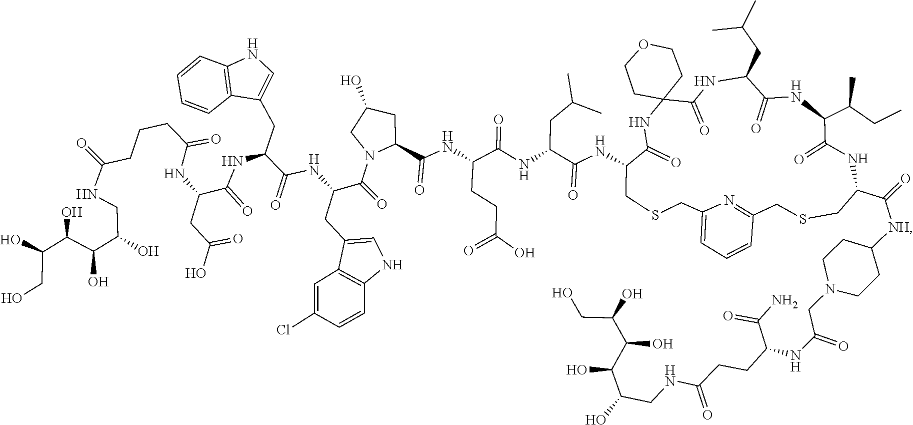 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CC(=O)O)NC(=O)CCCC(=O)NC[C@H](O)[C@@H](O)[C@H](O)[C@H](O)CO)CSCc2cccc(n2)CSC[C@@H](C(=O)NC2CCN(CC(=O)N[C@H](CCC(=O)NC[C@H](O)[C@@H](O)[C@H](O)[C@H](O)CO)C(N)=O)CC2)NC1=O]

compound Ac-Asp-Trp-5Clw-Tap (AGLU-Glutar)-Glu-leu-[Cys(2Lut)-Thp-Leu-Ile-Cys]-NH₂ (GIP-175, SEQ ID NO: 176) of the following formula

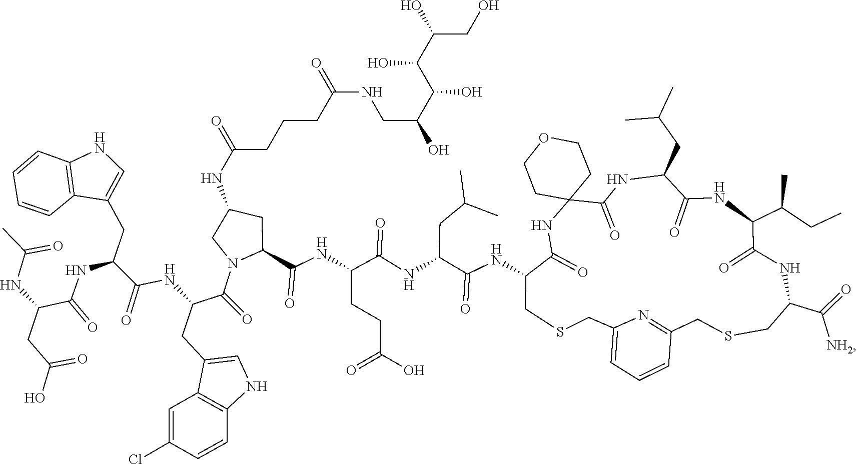 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](NC(=O)CCCC(=O)NC[C@H](O)[C@@H](O)[C@H](O)[C@H](O)CO)CN2C(=O)[C@H](Cc2c[nH]c3ccc(Cl)cc23)NC(=O)[C@H](Cc2c[nH]c3ccccc23)NC(=O)[C@H](CC(=O)O)NC(C)=O)CSCc2cccc(n2)CSC[C@@H](C(N)=O)NC1=O]

compound Ac-Asp-Trp-5Clw-Nglu(AGLU)-Glu-leu-[Cys(2Lut)-Thp-Leu-Ile-Cys]-NH₂ (GIP-176, SEQ ID NO: 177) of the following formula

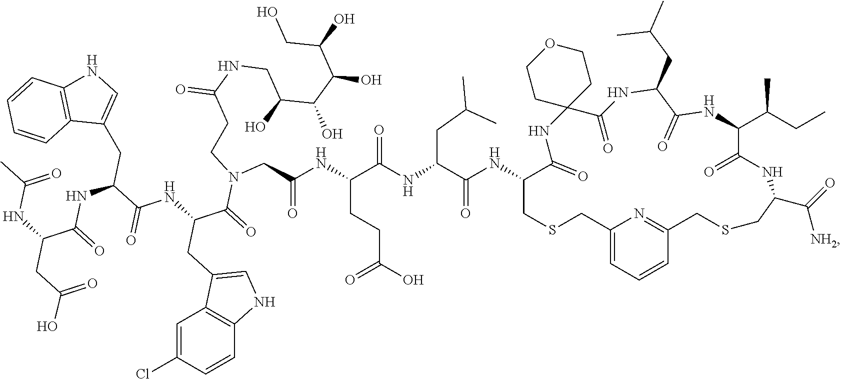

compound Ac-Asp-Trp-5Clw-Hyp-Glu-leu-[Cys(2Lut)-Thp-Leu-Ile-Cys]-APAc-glu(AGLU)-NH₂ (GIP-177, SEQ ID NO: 178) of the following formula

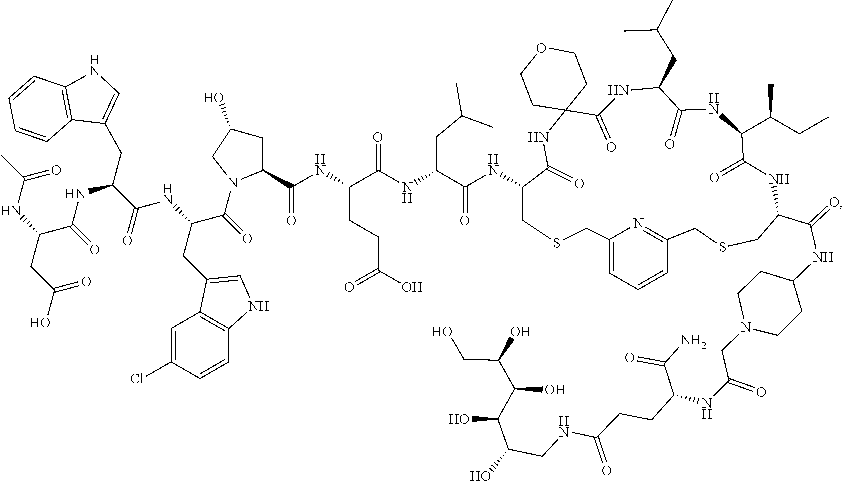 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CC(=O)O)NC(C)=O)CSCc2cccc(n2)CSC[C@@H](C(=O)NC2CCN(CC(=O)N[C@H](CCC(=O)NC[C@H](O)[C@@H](O)[C@H](O)[C@H](O)CO)C(N)=O)CC2)NC1=O]

compound AGLU-Glutar-Asp-Trp-5Clw-Hyp-Glu-leu-[Cys(2Lut)-Thp-Leu-Ile-Cys]-NH₂ (GIP-178, SEQ ID NO: 179) of the following formula

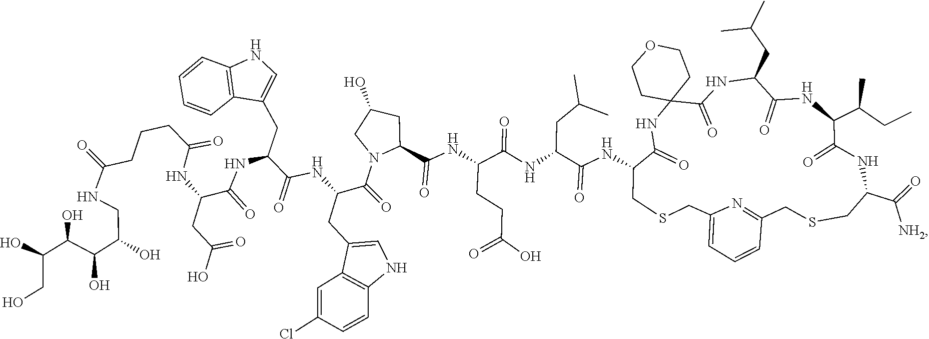 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CC(=O)O)NC(=O)CCCC(=O)NC[C@H](O)[C@@H](O)[C@H](O)[C@H](O)CO)CSCc2cccc(n2)CSC[C@@H](C(N)=O)NC1=O]

compound Ac-Cya-Trp-5Clw-Hyp-Glu-leu-[Cys(2Lut)-Aib-Leu-Ile-AET] (GIP-179, SEQ ID NO: 180) of the following formula

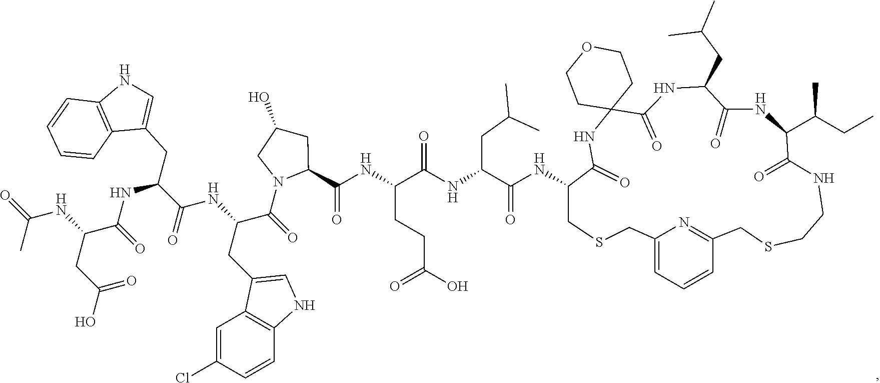 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CC(=O)O)NC(C)=O)CSCc2cccc(n2)CSCCNC1=O]

compound Ac-Asp-Trp-5Clw-Hyp-Glu-leu-[Cys(2Lut)-Thp-Leu-Ile-Cys]-NH₂ (GIP-180, SEQ ID NO: 181) of the following formula

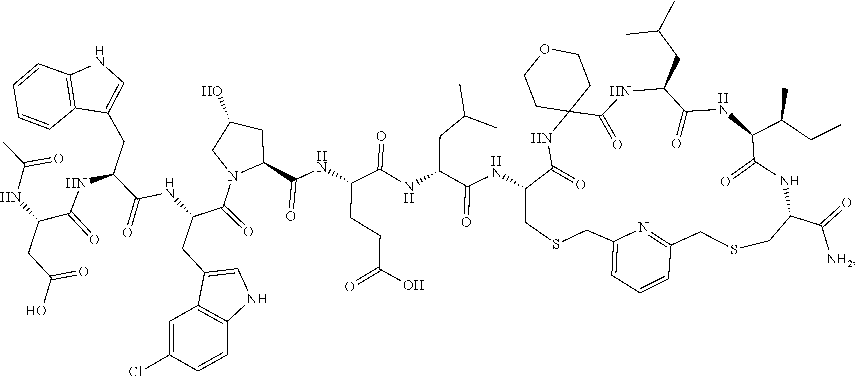 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CC(=O)O)NC(C)=O)CSCc2cccc(n2)CSC[C@@H](C(N)=O)NC1=O]

compound Ac-Asp-Trp-5Clw-Hyp-Glu-leu-[Cys(2Lut)-Thp-Leu-Ile-Cys]-OH (GIP-181, SED ID NO: 182) of the following formula

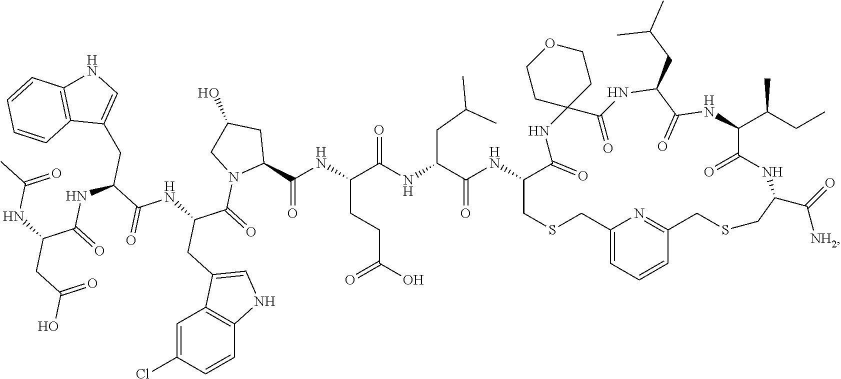 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CC(=O)O)NC(C)=O)CSCc2cccc(n2)CSC[C@@H](C(N)=O)NC1=O]

compound Ac-Asp-Trp-5Clw-Hyp-Glu-leu-[Cys(2Lut)-Thp-Leu-Ile-Cysol]-OH (GIP-182, SEQ ID NO: 183) of the following formula

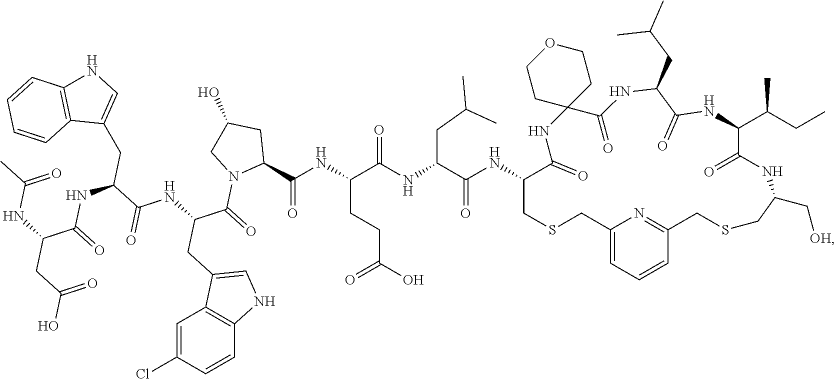 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CC(=O)O)NC(C)=O)CSCc2cccc(n2)CSC[C@@H](CO)NC1=O]

compound Ac-Asp-Trp-5Clw-Nmg-Glu-leu-[Cys(2Lut)-Thp-Leu-Ile-Cys]-NH₂ (GIP-183, SED ID NO; 184) of the following formula

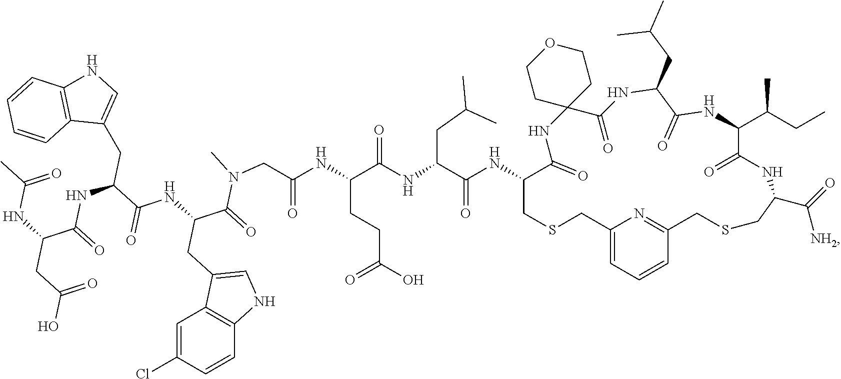 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)CN(C)C(=O)[C@H](Cc2c[nH]c3ccc(Cl)cc23)NC(=O)[C@H](Cc2c[nH]c3ccccc23)NC(=O)[C@H](CC(=O)O)NC(C)=O)CSCc2cccc(n2)CSC[C@@H](C(N)=O)NC1=O]

compound Ac-Asp-Trp-5Clw-Nleu-Glu-leu-[Cys(2Lut)-Thp-Leu-Ile-Cys]-NH₂ (GIP-184, SEQ ID NO: 185) of the following formula

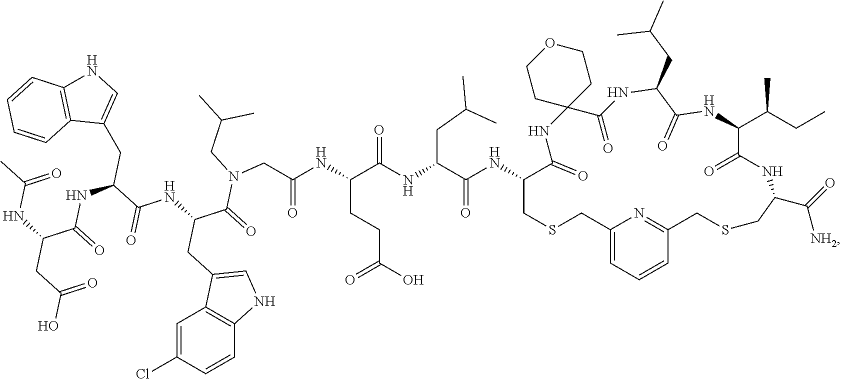 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)CN(CC(C)C)C(=O)[C@H](Cc2c[nH]c3ccc(Cl)cc23)NC(=O)[C@H](Cc2c[nH]c3ccccc23)NC(=O)[C@H](CC(=O)O)NC(C)=O)CSCc2cccc(n2)CSC[C@@H](C(N)=O)NC1=O]

compound Ac-Asp-Trp-5Clw-Nphe-Glu-leu-[Cys(2Lut)-Thp-Leu-Ile-Cys]-NH₂ (GIP-185, SEQ ID NO: 186) of the following formula

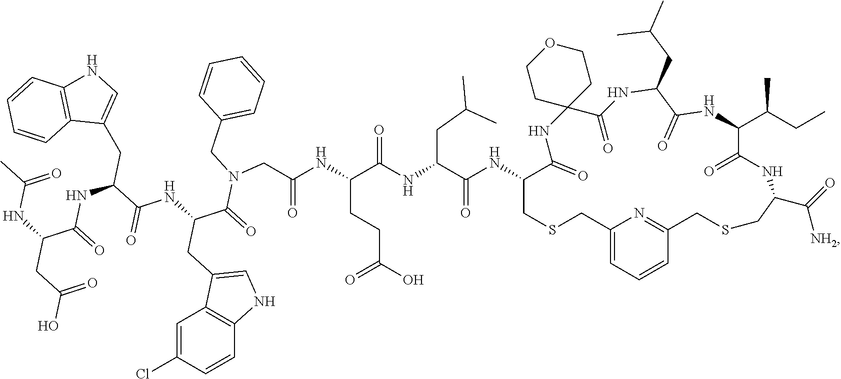 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)CN(Cc2ccccc2)C(=O)[C@H](Cc2c[nH]c3ccc(Cl)cc23)NC(=O)[C@H](Cc2c[nH]c3ccccc23)NC(=O)[C@H](CC(=O)O)NC(C)=O)CSCc2cccc(n2)CSC[C@@H](C(N)=O)NC1=O]

compound Ac-Asp-Trp-5Clw-Nlys-Glu-leu-[Cys(2Lut)-Thp-Leu-Ile-Cys]-NH₂ (GIP-186, SEQ ID NO: 187) of the following formula

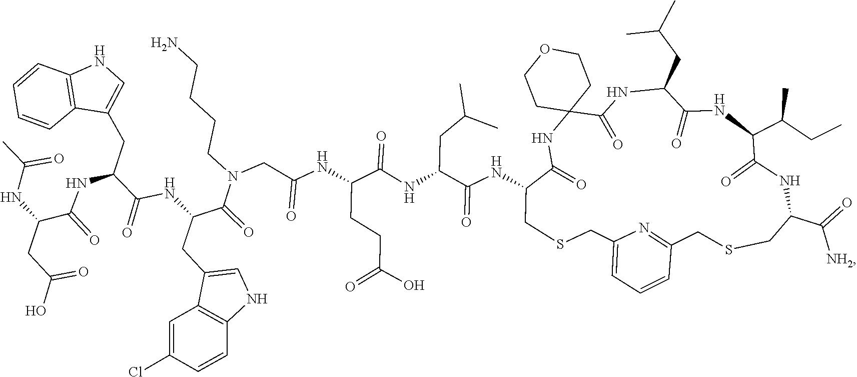 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)CN(CCCCN)C(=O)[C@H](Cc2c[nH]c3ccc(Cl)cc23)NC(=O)[C@H](Cc2c[nH]c3ccccc23)NC(=O)[C@H](CC(=O)O)NC(C)=O)CSCc2cccc(n2)CSC[C@@H](C(N)=O)NC1=O]

compound Ac-Asp-Trp-5Clw-4Tfp-Glu-leu-[Cys(2Lut)-Thp-Leu-Ile-Cys]-NH₂ (GIP-187, SEQ ID NO: 188) of the following formula

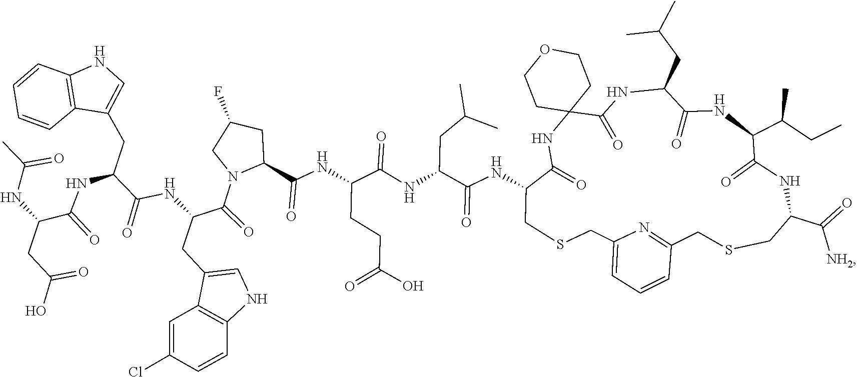 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](F)CN2C(=O)[C@H](Cc2c[nH]c3ccc(Cl)cc23)NC(=O)[C@H](Cc2c[nH]c3ccccc23)NC(=O)[C@H](CC(=O)O)NC(C)=O)CSCc2cccc(n2)CSC[C@@H](C(N)=O)NC1=O]

compound Ac-Ser-Trp-5Clw-Hyp-Glu-leu-[Cys(2Lut)-Thp-Leu-Ile-Cys]-NH₂ (GIP-188, SEQ ID NO: 189) of the following formula

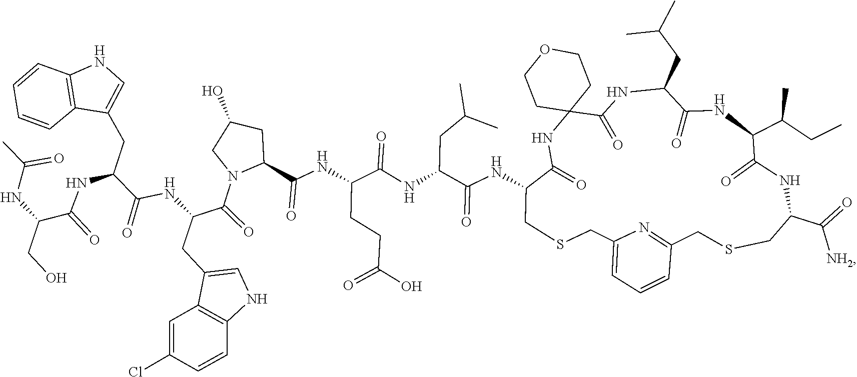 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CO)NC(C)=O)CSCc2cccc(n2)CSC[C@@H](C(N)=O)NC1=O]

compound Ac-Cya-Trp-5Clw-Hyp-Glu-leu-[Cys(2Lut)-Thp-Leu-Ile-Cys]-NH₂ (GIP-189, SEQ ID NO: 190) of the following formula

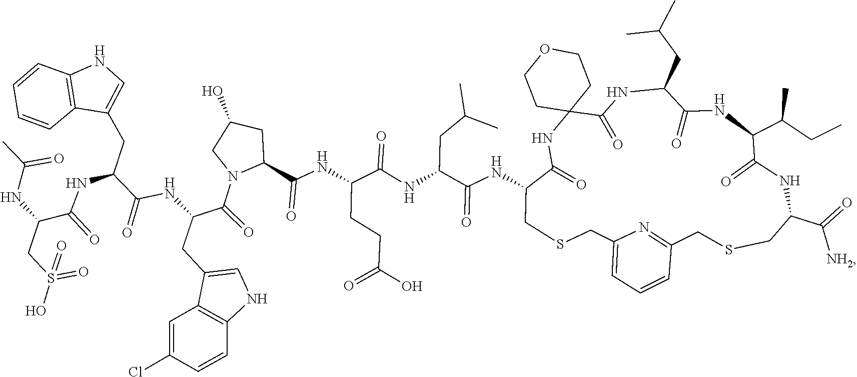 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CS(=O)(=O)O)NC(C)=O)CSCc2cccc(n2)CSC[C@@H](C(N)=O)NC1=O]

compound Ac-Asn-Trp-5Clw-Hyp-Glu-leu-[Cys(2Lut)-Thp-Leu-Ile-Cys]-NH₂ (GIP-190, SEQ ID NO: 191) of the following formula

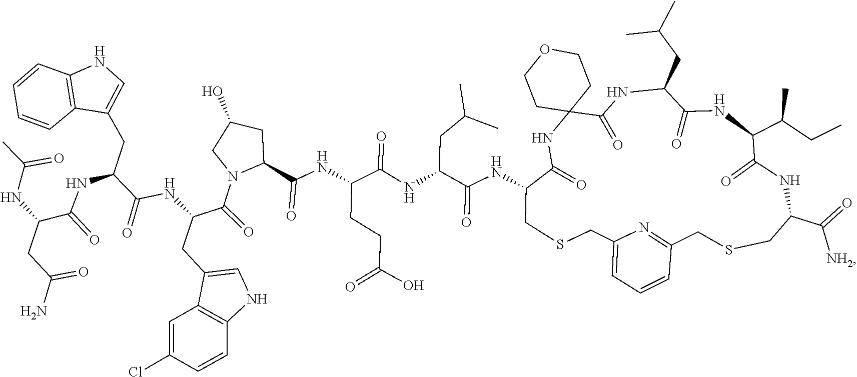 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CC(N)=O)NC(C)=O)CSCc2cccc(n2)CSC[C@@H](C(N)=O)NC1=O]

compound Ac-Asn-Trp-5Clw-Hyp-Glu-leu-[Cys(2Lut)-Thp-Leu-Ile-Cys]-NH₂ (GIP-191, SEQ ID NO: 192) of the following formula

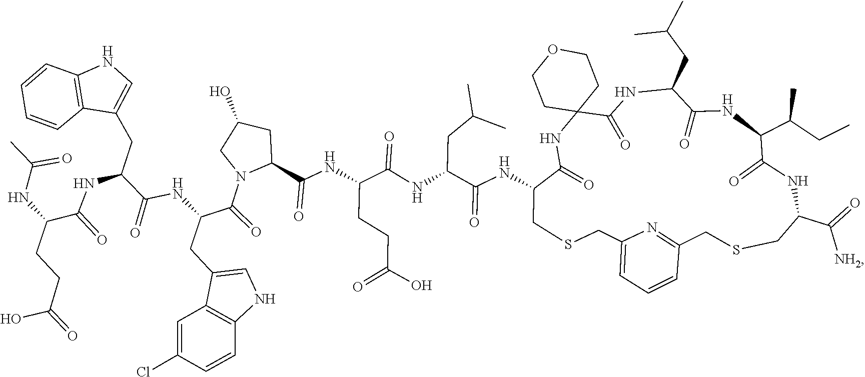 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CCC(=O)O)NC(C)=O)CSCc2cccc(n2)CSC[C@@H](C(N)=O)NC1=O]

compound Ac-Asp-Trp-5Clw-Hyp-Asp-leu-[Cys(2Lut)-Thp-Leu-Ile-Cys]-NH₂ (GIP-192, SEQ ID NO: 193) of the following formula

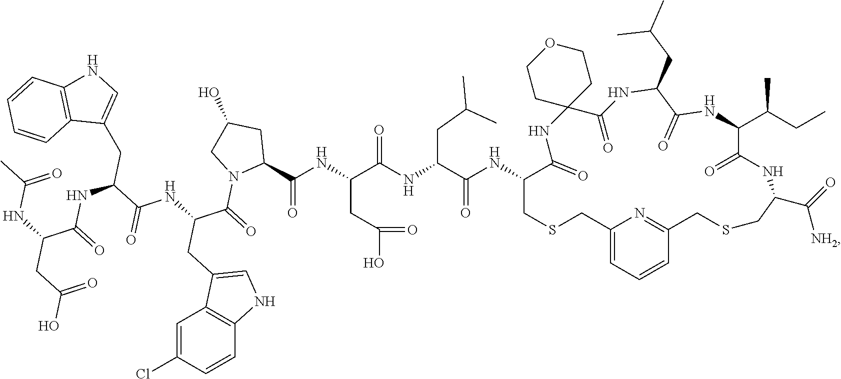 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CC(=O)O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CC(=O)O)NC(C)=O)CSCc2cccc(n2)CSC[C@@H](C(N)=O)NC1=O]

compound Ac-Asp-Trp-5Clw-Hyp-Gln-leu-[Cys(2Lut)-Thp-Leu-Ile-Cys]-NH₂ (GIP-193, SEQ ID NO: 194) of the following formula

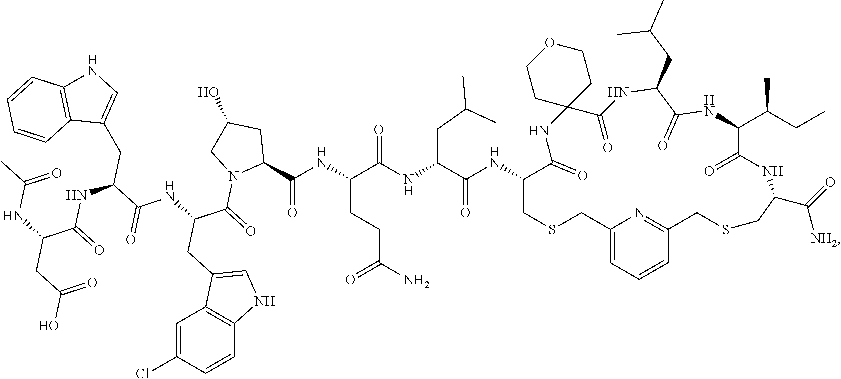 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(N)=O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CC(=O)O)NC(C)=O)CSCc2cccc(n2)CSC[C@@H](C(N)=O)NC1=O]

compound Ac-Asp-Trp-5Clw-Hyp-Hse-leu-[Cys(2Lut)-Thp-Leu-Ile-Cys]-NH₂ (GIP-194, SEQ ID NO: 195 of the following formula

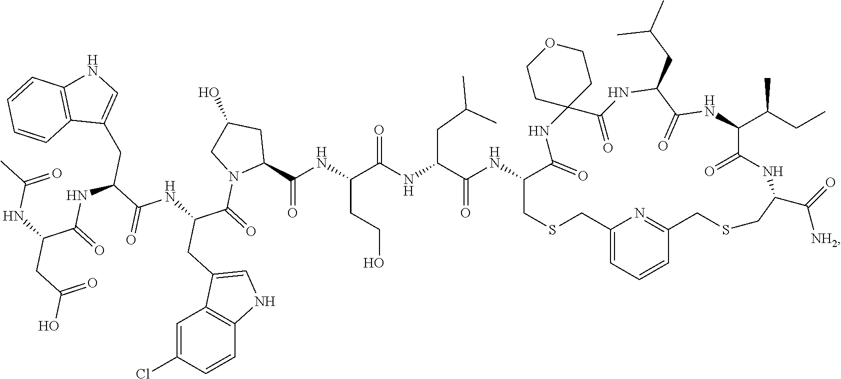 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCO)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CC(=O)O)NC(C)=O)CSCc2cccc(n2)CSC[C@@H](C(N)=O)NC1=O]

compound Ac-Hse-Trp-5Clw-Hyp-Glu-leu-[Cys(2Lut)-Thp-Leu-Ile-Cys]-NH₂ (GIP-195, SEQ ID NO: 196) of the following formula

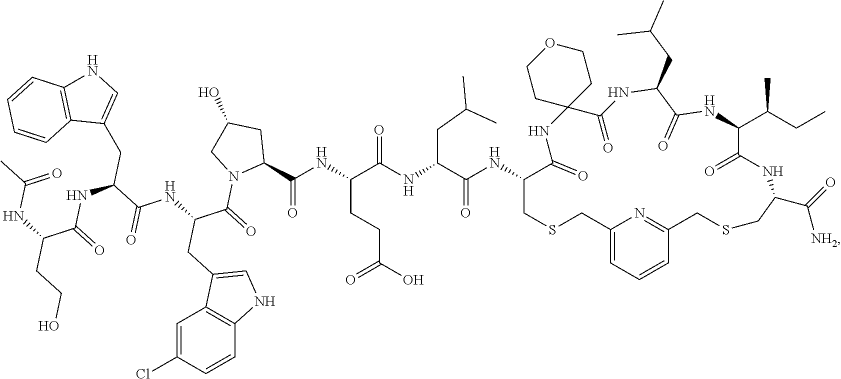 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CCO)NC(C)=O)CSCc2cccc(n2)CSC[C@@H](C(N)=O)NC1=O]

compound Ac-Asp-Trp-5Clw-Nglu-Glu-leu-[Cys(2Lut)-Thp-Leu-Ile-Cys]-NH₂ (GIP-196, SEQ ID NO: 197) of the following formula

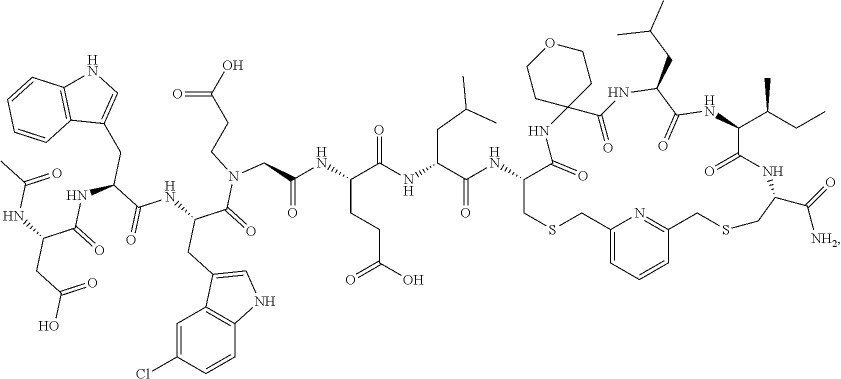

compound Ac-Hse-Trp-5Brw-Hyp-Glu-leu-[Cys(2Lut)-Thp-Leu-Ile-Cys]-NH₂ (GIP-197, SEQ ID NO: 198) of the following formula

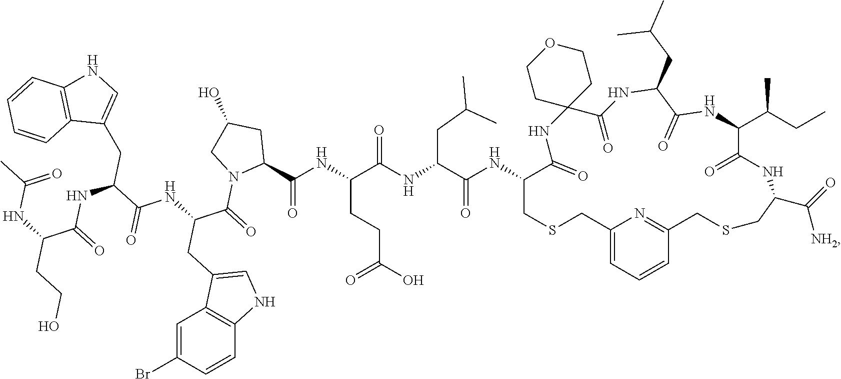 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Br)cc23)NC(=O)[C@H](Cc2c[nH]c3ccccc23)NC(=O)[C@H](CCO)NC(C)=O)CSCc2cccc(n2)CSC[C@@H](C(N)=O)NC1=O]

compound HO-Succinyl-Trp-5Clw-Pro-Glu-leu-[Cys(3MeBn)-Aib-Leu-Ile-Cys]-NH₂ (GIP-198, SEQ ID NO: 199) of the following formula

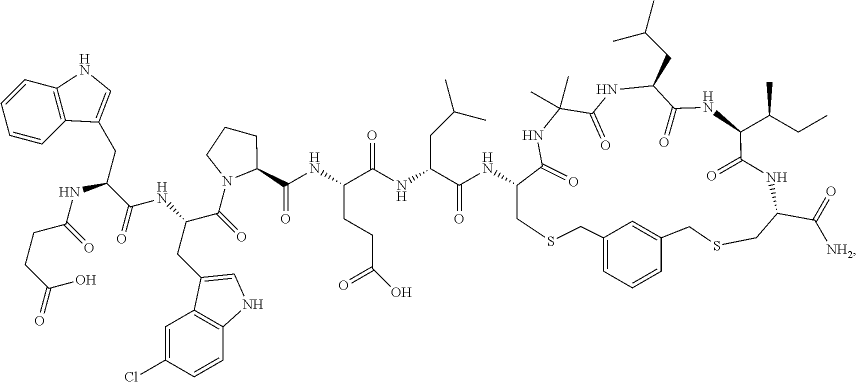 [SMILES: CC[C@H](C)[C@@H]1NC(=O)[C@H](CC(C)C)NC(=O)C(C)(C)NC(=O)[C@@H](NC(=O)[C@@H](CC(C)C)NC(=O)[C@H](CCC(=O)O)NC(=O)[C@@H]2CCCN2C(=O)[C@H](Cc2c[nH]c3ccc(Cl)cc23)NC(=O)[C@H](Cc2c[nH]c3ccccc23)NC(=O)CCC(=O)O)CSCc2cccc(c2)CSC[C@@H](C(N)=O)NC1=O]

compound HO-Succinyl-Trp-5Clw-Pro-Glu-leu-[Cys(3MeBn)-Aib-Leu-Ile-Cys]-NH₂ (GIP-199, SEQ ID NO: 200) of the following formula

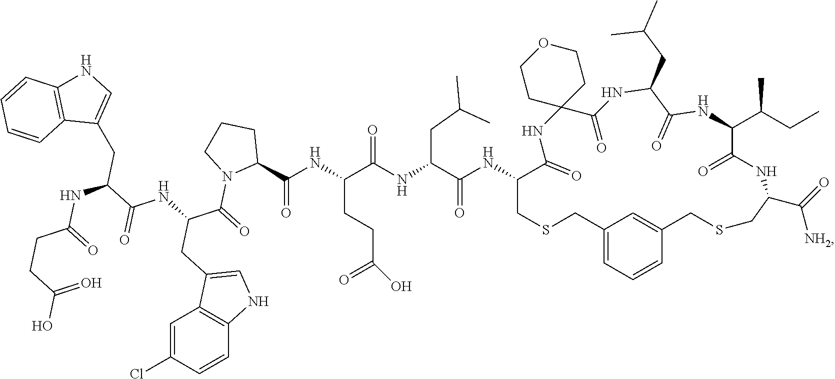 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2CCCN2C(=O)[C@H](Cc2c[nH]c3ccc(Cl)cc23)NC(=O)[C@H](Cc2c[nH]c3ccccc23)NC(=O)CCC(=O)O)CSCc2cccc(c2)CSC[C@@H](C(N)=O)NC1=O]

compound Ac-Asp-Trp-5Clw-Hyp-Glu-leu-[Hcy(2Lut)-Thp-Leu-Ile-Cys]-NH₂ (GIP-200, SEQ ID NO: 201) of the following formula

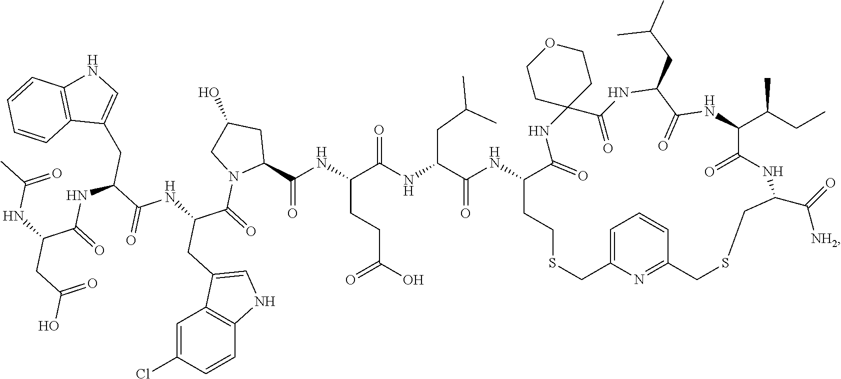 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CC(=O)O)NC(C)=O)CCSCc2cccc(n2)CSC[C@@H](C(N)=O)NC1=O]

compound Ac-Asp-Trp-5Clw-Hyp-Glu-leu-[Cys(2Lut)-Thp-Leu-Ile-Hcy]-NH₂ (GIP-201, SEQ ID NO: 202) of the following formula

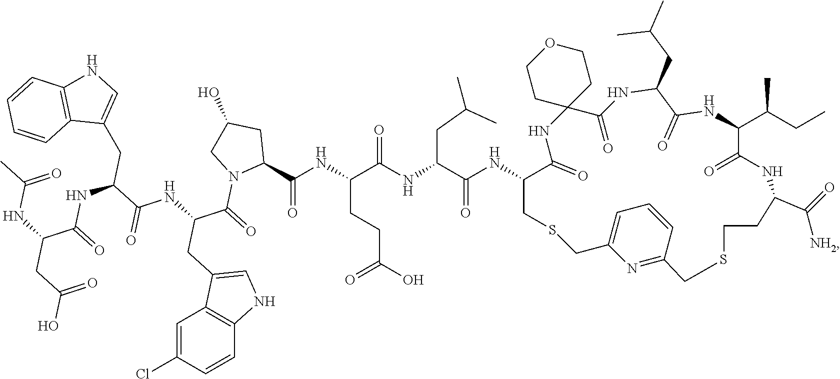 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CC(=O)O)NC(C)=O)CSCc2cccc(n2)CSCC[C@@H](C(N)=O)NC1=O]

compound Ac-Asp-Trp-5Clw-Hyp-Glu-leu-[Hcy(2Lut)-Thp-Leu-Ile-Hcy]-NH₂ (GIP-202, SEQ ID NO: 203) of the following formula

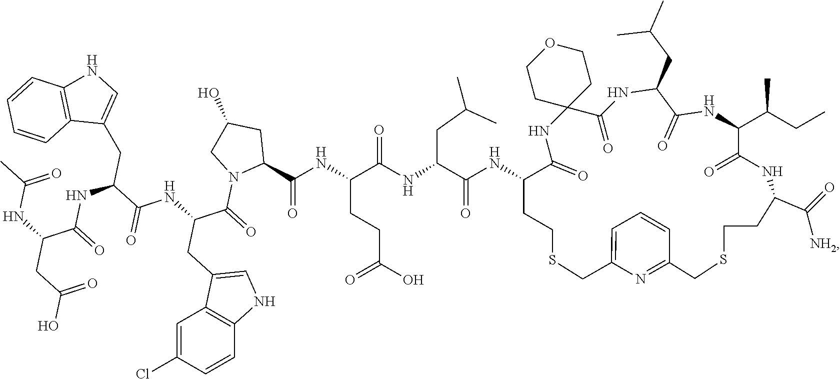 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CC(=O)O)NC(C)=O)CCSCc2cccc(n2)CSCC[C@@H](C(N)=O)NC1=O]

compound Ac-Asp-Trp-5Clw-Hyp-Glu-leu-[Cys(2Lut)-Eca-Leu-Ile-Cys]-NH₂ (GIP-203, SEQ ID NO: 204) of the following formula

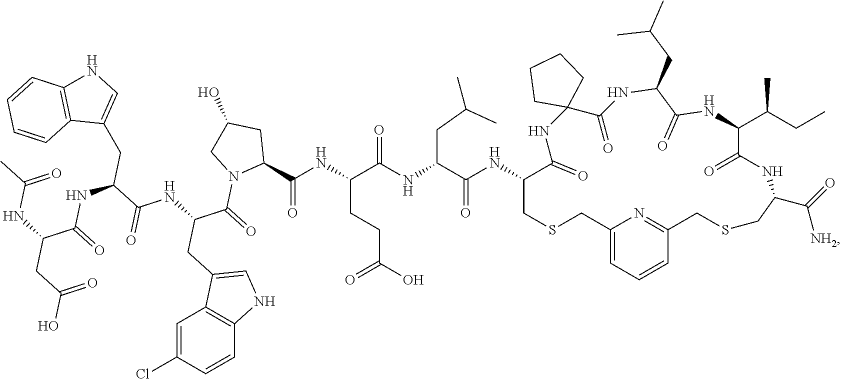 [SMILES: CC[C@H](C)[C@@H]1NC(=O)[C@H](CC(C)C)NC(=O)C2(CCCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CC(=O)O)NC(C)=O)CSCc2cccc(n2)CSC[C@@H](C(N)=O)NC1=O]

compound Ac-Gln-Trp-5Clw-Hyp-Glu-leu-[Cys(2Lut)-Thp-Leu-Ile-Cys]-NH₂ (GIP-204, SEQ ID NO: 205) of the following formula

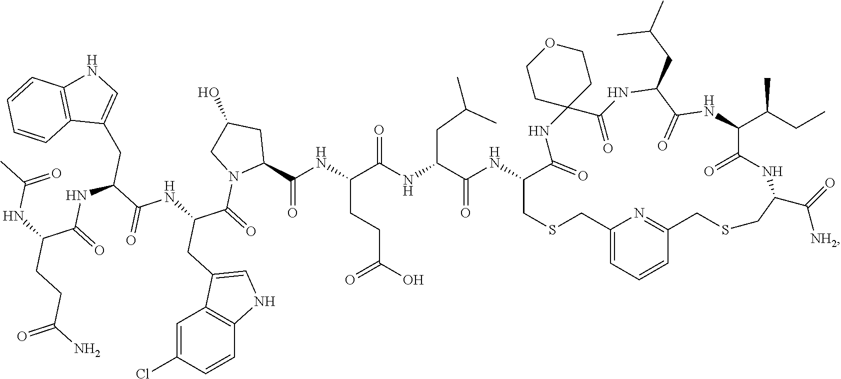 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CCC(N)=O)NC(C)=O)CSCc2cccc(n2)CSC[C@@H](C(N)=O)NC1=O]

compound Ac-Asp-Trp-5Clw-Hyp-Asn-leu-[Cys(2Lut)-Thp-Leu-Ile-Cys]-NH₂ (GIP-205, SEQ ID NO: 206) of the following formula

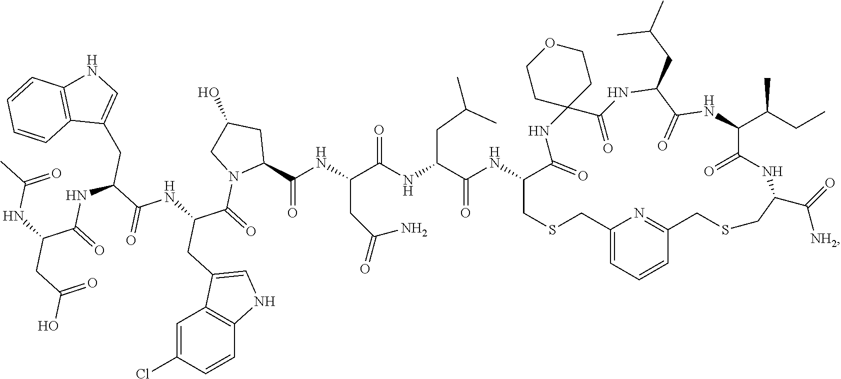 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CC(N)=O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CC(=O)O)NC(C)=O)CSCc2cccc(n2)CSC[C@@H](C(N)=O)NC1=O]

compound Ac-Asp-Trp-5Clw-Hyp-Glu-leu-[Cys(2Lut)-Aic-Leu-Ile-Cys]-NH₂ (GIP-206, SEQ ID NO: 207) of the following formula

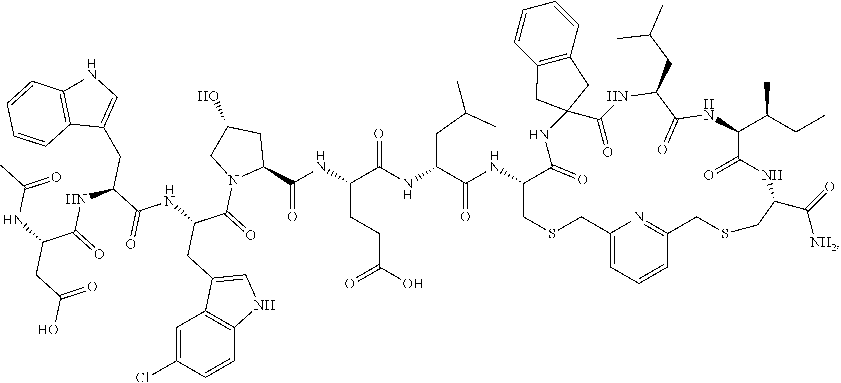 [SMILES: CC[C@H](C)[C@@H]1NC(=O)[C@H](CC(C)C)NC(=O)C2(Cc3ccccc3C2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CC(=O)O)NC(C)=O)CSCc2cccc(n2)CSC[C@@H](C(N)=O)NC1=O]

compound Ac-Pse-Trp-5Clw-Hyp-Glu-leu-[Cys(2Lut)-Thp-Leu-Ile-Cys]-NH₂ (GIP-207, SEQ ID NO: 208) of the following formula

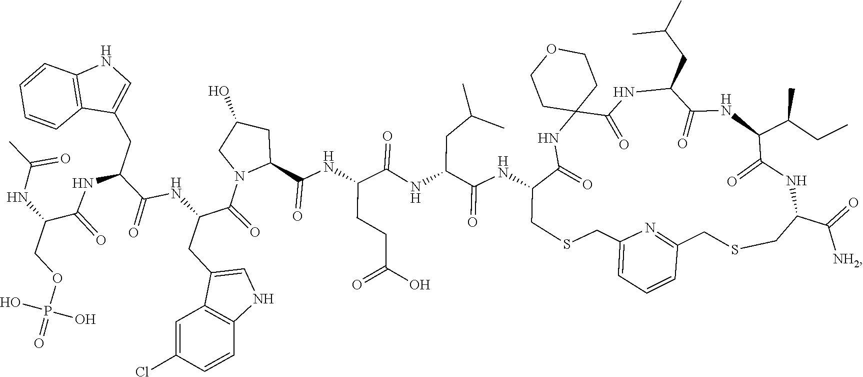 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](COP(=O)(O)O)NC(C)=O)CSCc2cccc(n2)CSC[C@@H](C(N)=O)NC1=O]

compound Ac-Asp-Trp-5Clw-Hyp-Pse-leu-[Cys(2Lut)-Thp-Leu-Ile-Cys]-NH₂ (GIP-208, SEQ ID NO: 209) of the following formula

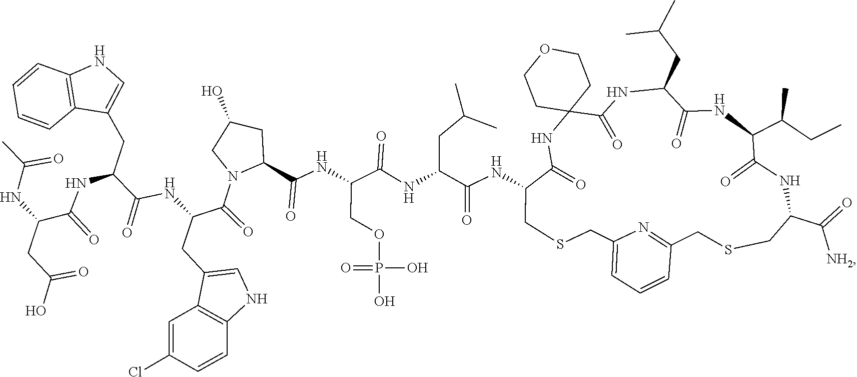 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](COP(=O)(O)O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CC(=O)O)NC(C)=O)CSCc2cccc(n2)CSC[C@@H](C(N)=O)NC1=O]

compound Ac-Asp-Mcf-5Clw-Hyp-Glu-leu-[Cys(2Lut)-Thp-Leu-Ile-Cys]-NH₂ (GIP-209, SEQ ID NO: 210) of the following formula

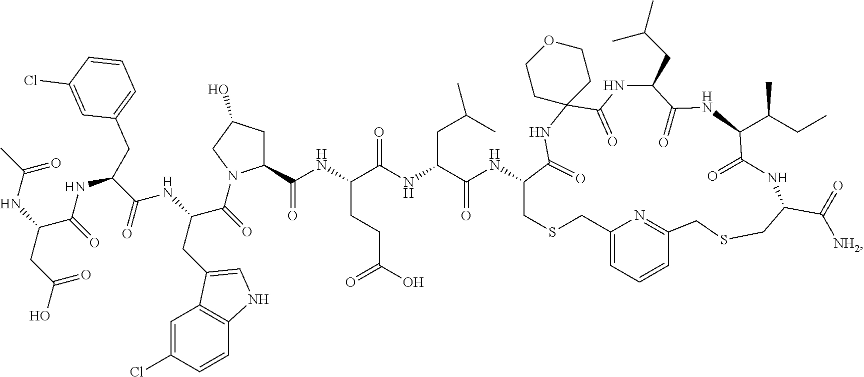 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2cccc(Cl)c2)NC(=O)[C@H](CC(=O)O)NC(C)=O)CSCc2cccc(n2)CSC[C@@H](C(N)=O)NC1=O]

compound Ac-Asp-Pcf-5Clw-Hyp-Glu-leu-[Cys(2Lut)-Thp-Leu-Ile-Cys]-NH₂ (GIP-210, SEQ ID NO: 211) of the following formula

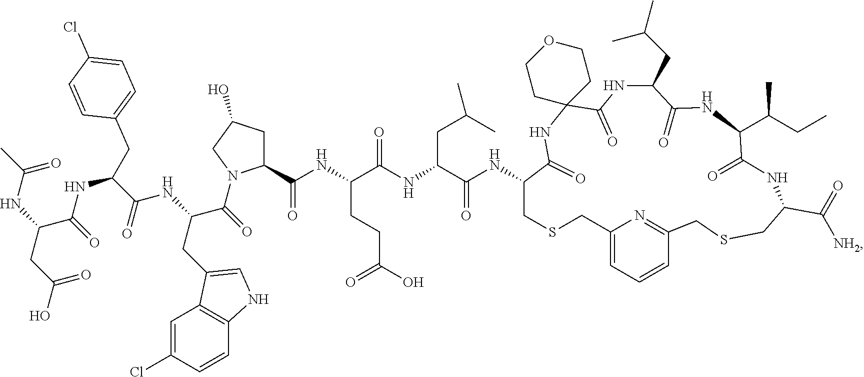 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2ccc(Cl)cc2)NC(=O)[C@H](CC(=O)O)NC(C)=O)CSCc2cccc(n2)CSC[C@@H](C(N)=O)NC1=O]

compound Ac-Asp-Eaa-5Clw-Hyp-Glu-leu-[Cys(2Lut)-Thp-Leu-Ile-Cys]-NH₂ (GIP-211, SEQ ID NO: 212) of the following formula

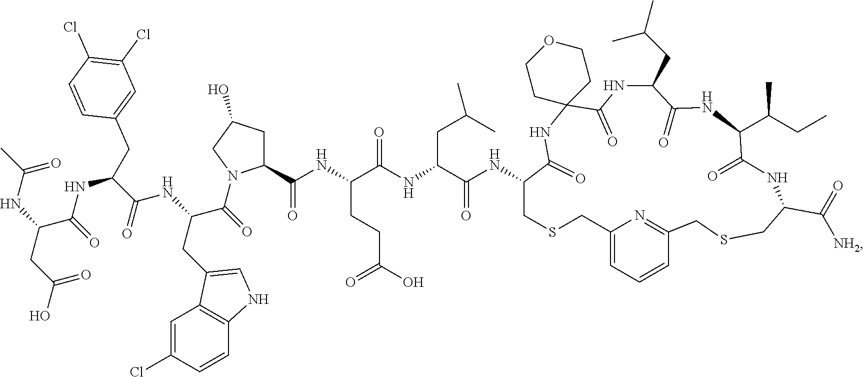 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2ccc(Cl)c(Cl)c2)NC(=O)[C@H](CC(=O)O)NC(C)=O)CSCc2cccc(n2)CSC[C@@H](C(N)=O)NC1=O]

compound Ac-Asp-Egm-5Clw-Hyp-Glu-leu-[Cys(2Lut)-Thp-Leu-Ile-Cys]-NH₂ (GIP-212, SEQ ID NO: 213) of the following formula

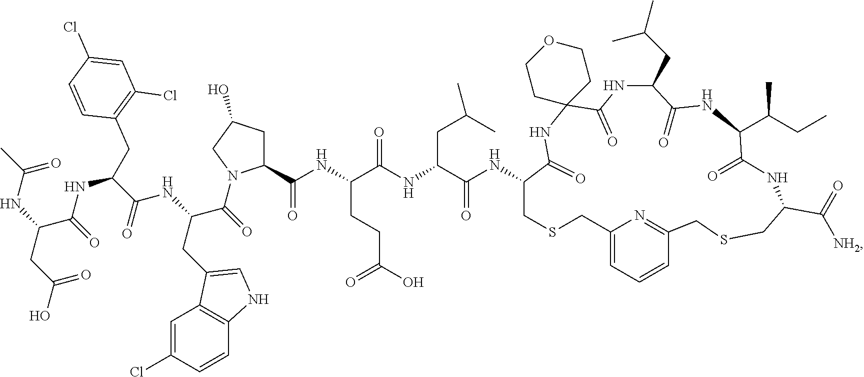 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2ccc(Cl)cc2Cl)NC(=O)[C@H](CC(=O)O)NC(C)=O)CSCc2cccc(n2)CSC[C@@H](C(N)=O)NC1=O]

compound Ac-Asp-Trp-Eaa-Hyp-Glu-leu-[Cys(2Lut)-Thp-Leu-Ile-Cys]-NH₂ (GIP-213, SEQ ID NO: 214) of the following formula

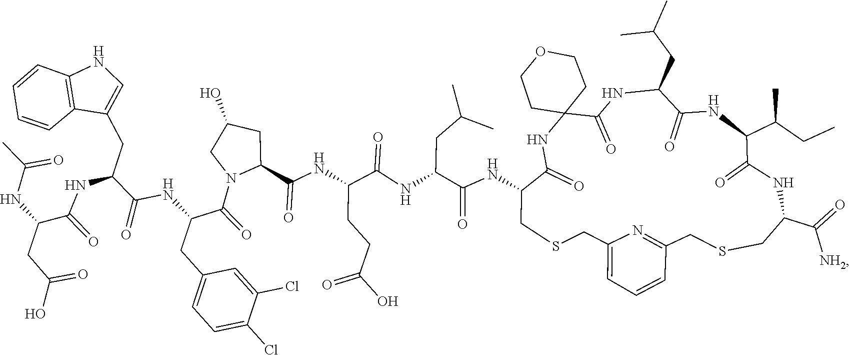 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2ccc(Cl)c(Cl)c2)NC(=O)[C@H](Cc2c[nH]c3ccccc23)NC(=O)[C@H](CC(=O)O)NC(C)=O)CSCc2cccc(n2)CSC[C@@H](C(N)=O)NC1=O]

compound HO-Glutar-Trp-5Clw-Hyp-Glu-leu-[Cys(2Lut)-Thp-Leu-Ile-Cys]-NH₂ (GIP-214, SEQ ID NO: 215) of the following formula

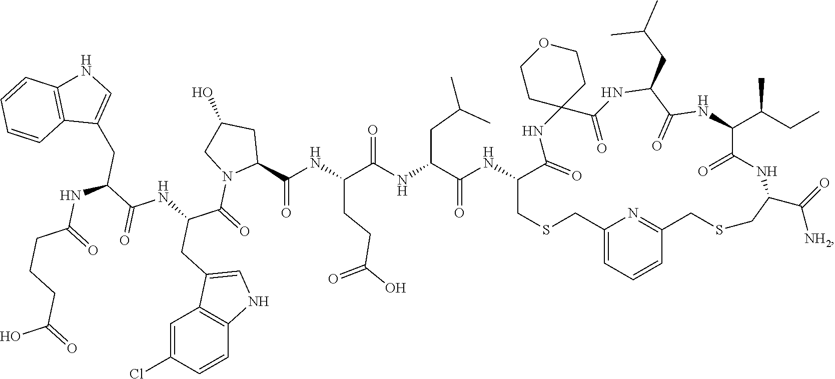 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)CCCC(=O)O)CSCc2cccc(n2)CSC[C@@H](C(N)=O)NC1=O]

compound Ac-Asp-Trp-5Clw-Dtc-Glu-leu-[Cys(2Lut)-Thp-Leu-Ile-Cys]-NH₂ (GIP-215, SEQ ID NO: 216) of the following formula

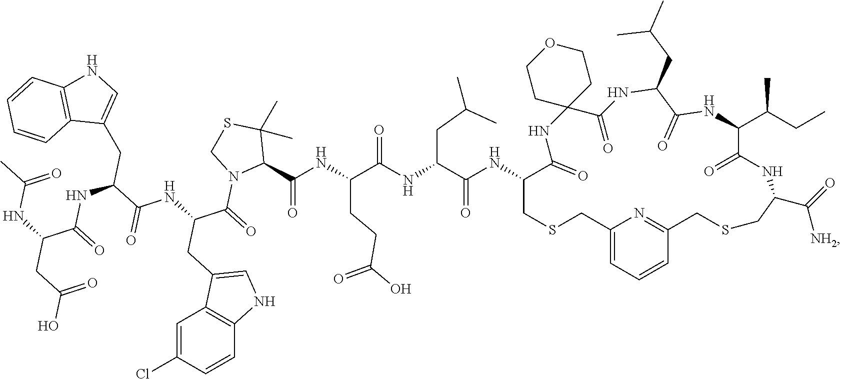 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@H]2N(C(=O)[C@H](Cc3c[nH]c4ccc(Cl)cc34)NC(=O)[C@H](Cc3c[nH]c4ccccc34)NC(=O)[C@H](CC(=O)O)NC(C)=O)CSC2(C)C)CSCc2cccc(n2)CSC[C@@H](C(N)=O)NC1=O]

compound Ac-Asp-Trp-5Clw-Oxa-Glu-leu-[Cys(2Lut)-Thp-Leu-Ile-Cys]-NH₂ (GIP-216, SEQ ID NO: 217) of the following formula

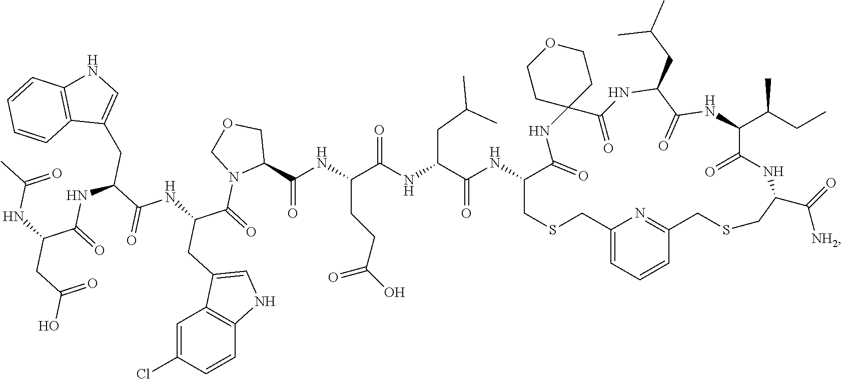 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2COCN2C(=O)[C@H](Cc2c[nH]c3ccc(Cl)cc23)NC(=O)[C@H](Cc2c[nH]c3ccccc23)NC(=O)[C@H](CC(=O)O)NC(C)=O)CSCc2cccc(n2)CSC[C@@H](C(N)=O)NC1=O]

compound Ac-Asp-Trp-5Clw-Hyp-Nme-Leu-[Cys(2Lut)-Thp-Leu-Ile-Cys]-NH₂ (GIP-217, SEQ ID NO: 218) of the following formula

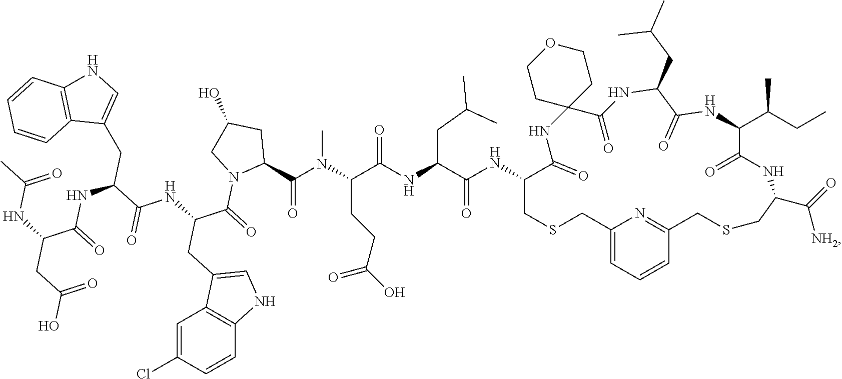 [SMILES: CC[C@H](C)[C@@H]1NC(=O)[C@H](CC(C)C)NC(=O)C2(CCOCC2)NC(=O)[C@@H](NC(=O)[C@H](CC(C)C)NC(=O)[C@H](CCC(=O)O)N(C)C(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CC(=O)O)NC(C)=O)CSCc2cccc(n2)CSC[C@@H](C(N)=O)NC1=O]

compound Ac-Asp-Trp-5Clw-Hyp-Glu-Ile-[Cys(2Lut)-Thp-Leu-Ile-Cys]-NH₂ (GIP-218, SEQ ID NO: 219) of the following formula

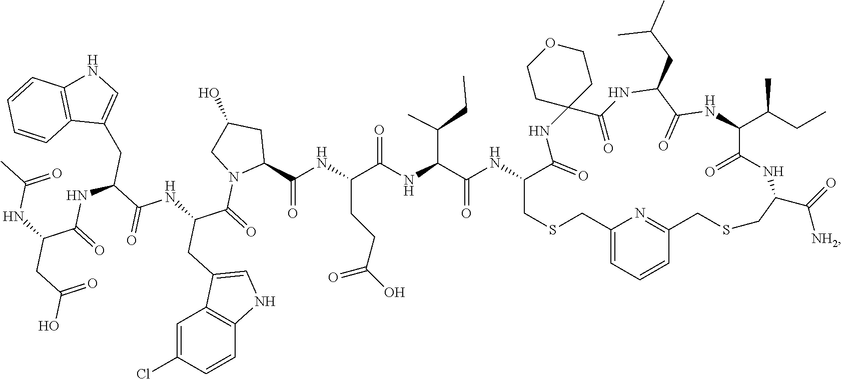 [SMILES: CC[C@H](C)[C@@H]1NC(=O)[C@H](CC(C)C)NC(=O)C2(CCOCC2)NC(=O)[C@@H](NC(=O)[C@@H](NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CC(=O)O)NC(C)=O)[C@@H](C)CC)CSCc2cccc(n2)CSC[C@@H](C(N)=O)NC1=O]

compound Ac-Asp-Trp-5Clw-Hyp-Glu-Val-[Cys(2Lut)-Thp-Leu-Ile-Cys]-NH₂ (GIP-219, SEQ ID NO: 220) of the following formula

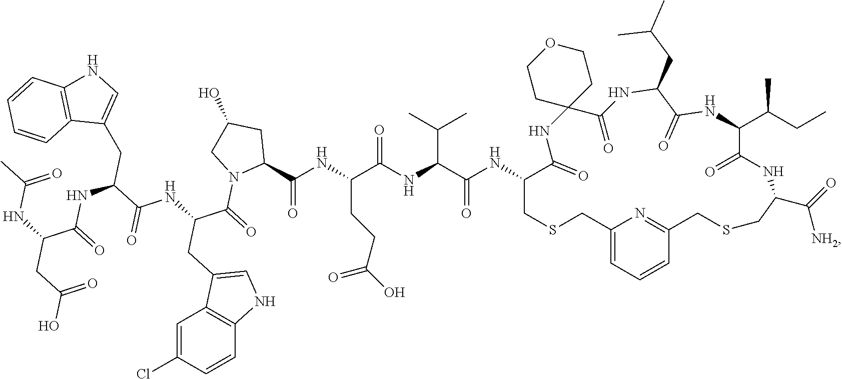 [SMILES: CC[C@H](C)[C@@H]1NC(=O)[C@H](CC(C)C)NC(=O)C2(CCOCC2)NC(=O)[C@@H](NC(=O)[C@@H](NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CC(=O)O)NC(C)=O)C(C)C)CSCc2cccc(n2)CSC[C@@H](C(N)=O)NC1=O]

compound Ac-Asp-Hyw-5Clw-Hyp-Glu-leu-[Cys(2Lut)-Thp-Leu-Ile-Cys]-NH₂ (GIP-220, SEQ ID NO: 221) of the following formula

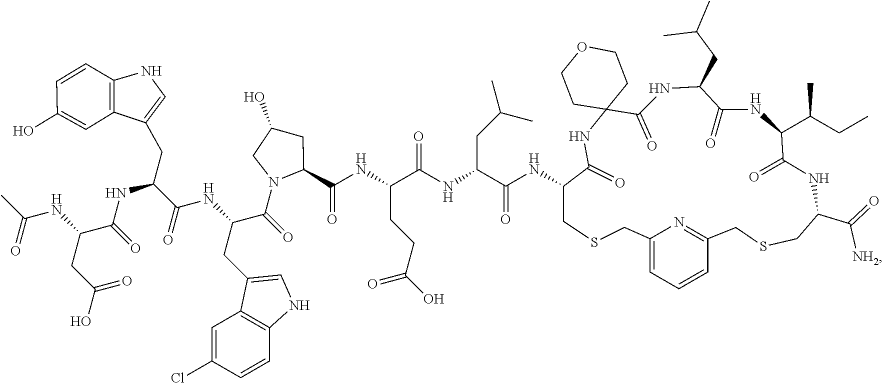 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3ccc(O)cc23)NC(=O)[C@H](CC(=O)O)NC(C)=O)CSCc2cccc(n2)CSC[C@@H](C(N)=O)NC1=O]

compound Ac-Asp-7Fw-5Clw-Hyp-Glu-leu-[Cys(2Lut)-Thp-Leu-Ile-Cys]-NH₂ (GIP-221, SEQ ID NO: 222) of the following formula

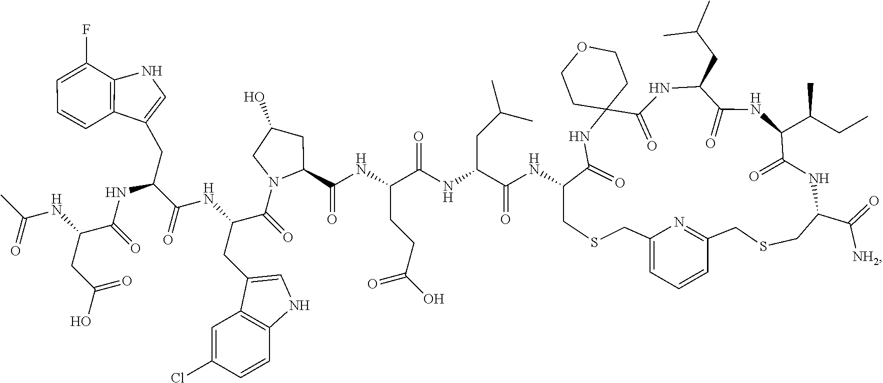 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3c(F)cccc23)NC(=O)[C@H](CC(=O)O)NC(C)=O)CSCc2cccc(n2)CSC[C@@H](C(N)=O)NC1=O]

compound Ac-Asp-Trp-5Clw-Eaz-Glu-leu-[Cys(2Lut)-Thp-Leu-Ile-Cys]-NH₂ (GIP-222, SEQ ID NO: 223) of the following formula

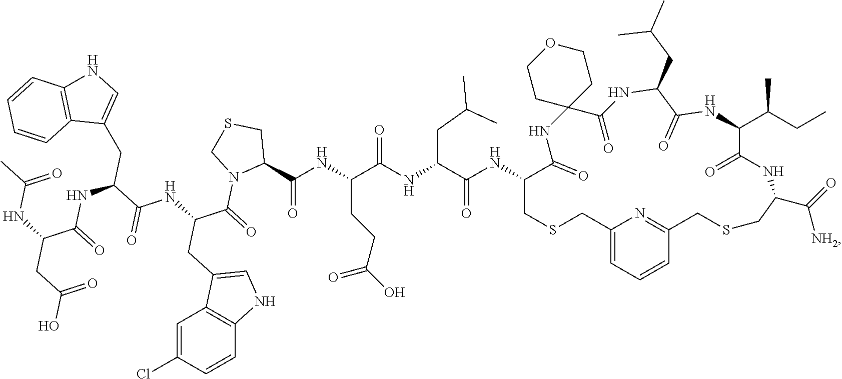 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2CSCN2C(=O)[C@H](Cc2c[nH]c3ccc(Cl)cc23)NC(=O)[C@H](Cc2c[nH]c3ccccc23)NC(=O)[C@H](CC(=O)O)NC(C)=O)CSCc2cccc(n2)CSC[C@@H](C(N)=O)NC1=O]

compound Ac-Asp-Trp-5Clw-Eaz-Glu-leu-[Cys(2Lut)-Thp-Leu-Ile-Cys]-NH₂ (GIP-223, SEQ ID NO: 224) of the following formula

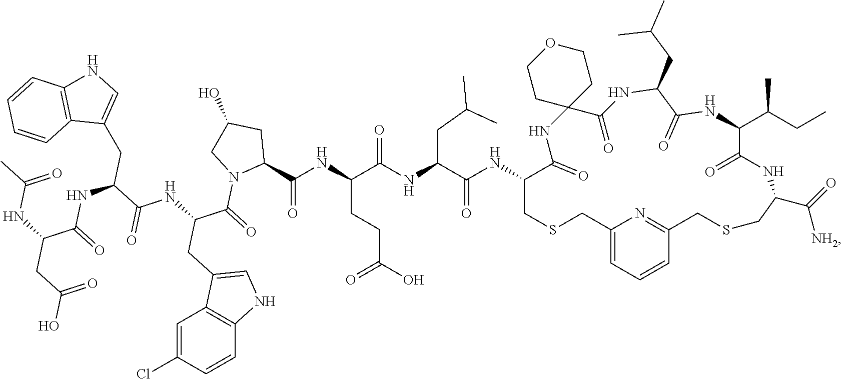 [SMILES: CC[C@H](C)[C@@H]1NC(=O)[C@H](CC(C)C)NC(=O)C2(CCOCC2)NC(=O)[C@@H](NC(=O)[C@H](CC(C)C)NC(=O)[C@@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CC(=O)O)NC(C)=O)CSCc2cccc(n2)CSC[C@@H](C(N)=O)NC1=O]

compound Ac-Asp-Phe-5Clw-Hyp-Glu-leu-[Cys(2Lut)-Thp-Leu-Ile-Cys]-NH₂ (GIP-224, SEQ ID NO: 225) of the following formula

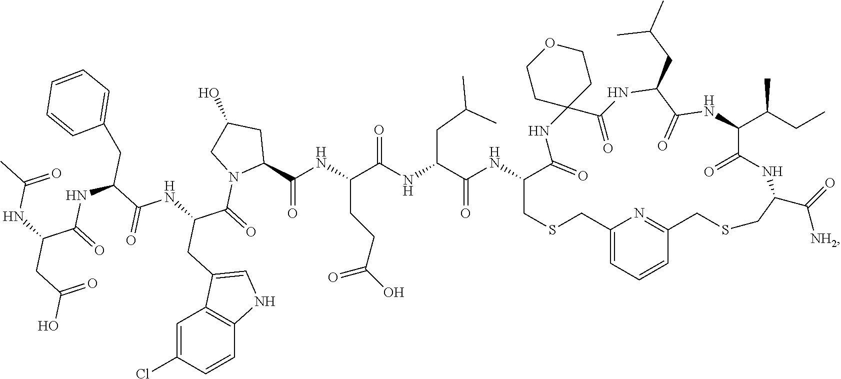 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2ccccc2)NC(=O)[C@H](CC(=O)O)NC(C)=O)CSCc2cccc(n2)CSC[C@@H](C(N)=O)NC1=O]

compound Ac-Asp-Egp-5Clw-Hyp-Glu-leu-[Cys(2Lut)-Thp-Leu-Ile-Cys]-NH₂ (GIP-226, SEQ ID NO: 226) of the following formula

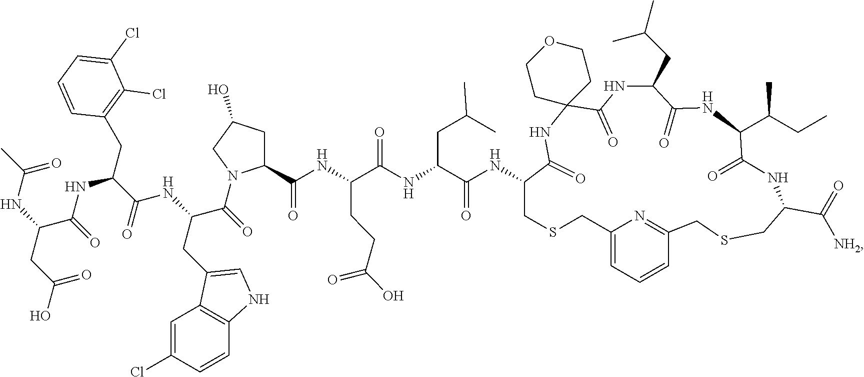 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2cccc(Cl)c2Cl)NC(=O)[C@H](CC(=O)O)NC(C)=O)CSCc2cccc(n2)CSC[C@@H](C(N)=O)NC1=O]

compound Ac-Asp-Trp-7Fw-Hyp-Glu-leu-[Cys(2Lut)-Thp-Leu-Ile-Cys]-NH₂ (GIP-227, SEQ ID NO: 227) of the following formula

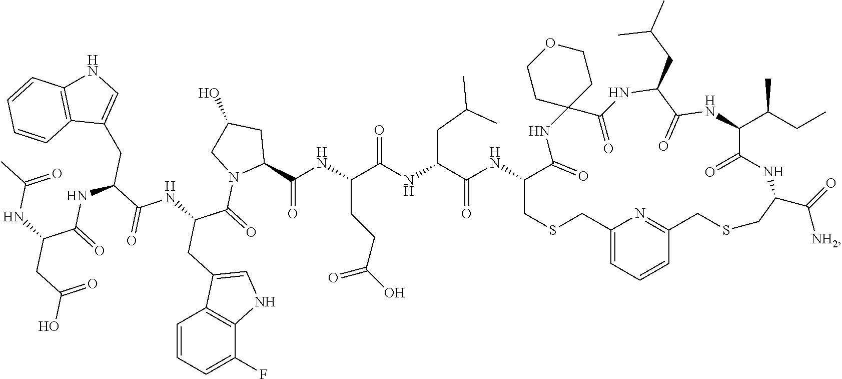 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3c(F)cccc23)NC(=O)[C@H](Cc2c[nH]c3ccccc23)NC(=O)[C@H](CC(=O)O)NC(C)=O)CSCc2cccc(n2)CSC[C@@H](C(N)=O)NC1=O]

compound Ac-Asp-Trp-5Clw-Hyp-Glu-leu-[Cys(2Lut)-Phe-Leu-Ile-Cys]-NH₂ (GIP-228, SEQ ID NO: 228) of the following formula

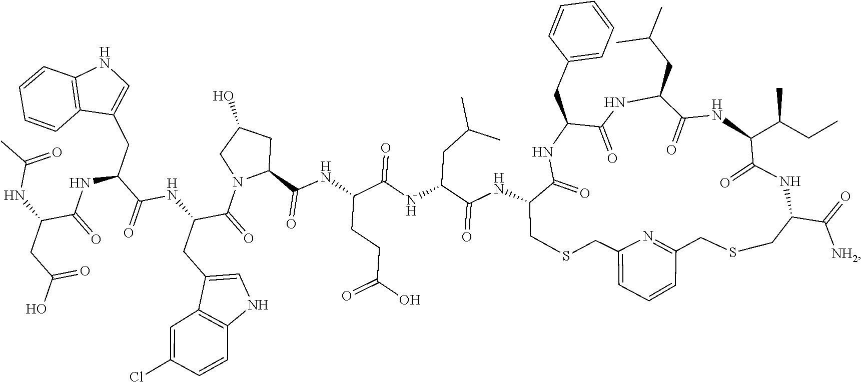 [SMILES: CC[C@H](C)[C@@H]1NC(=O)[C@H](CC(C)C)NC(=O)[C@H](Cc2ccccc2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CC(=O)O)NC(C)=O)CSCc2cccc(n2)CSC[C@@H](C(N)=O)NC1=O]

compound Ac-Asp-Trp-5Clw-Hyp-Glu-leu-[Cys(2Lut)-Thp-Ile-Ile-Cys]-NH₂ (GIP-229, SEQ ID NO: 229) of the following formula

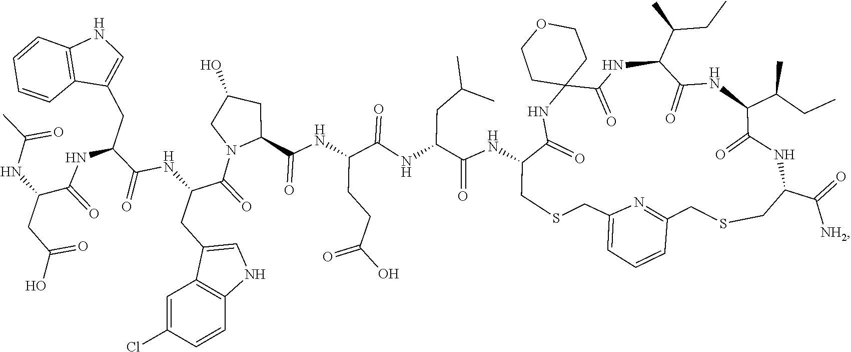 [SMILES: CC[C@H](C)[C@@H]1NC(=O)[C@H]([C@@H](C)CC)NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CC(=O)O)NC(C)=O)CSCc2cccc(n2)CSC[C@@H](C(N)=O)NC1=O]

compound Ac-Asp-Trp-5Clw-Hyp-Glu-leu-[Cys(2Lut)-Thp-Arg-Ile-Cys]-NH₂ (GIP-230, SEQ ID NO: 230) of the following formula

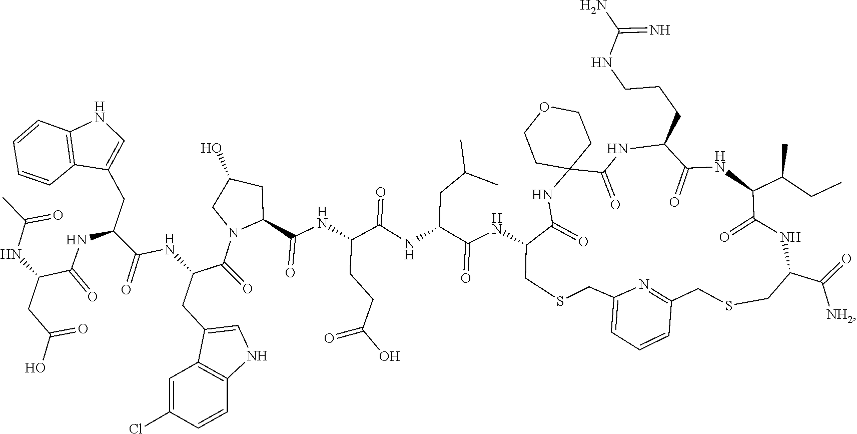 [SMILES: CC[C@H](C)[C@@H]1NC(=O)[C@H](CCCNC(=N)N)NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CC(=O)O)NC(C)=O)CSCc2cccc(n2)CSC[C@@H](C(N)=O)NC1=O]

compound Ac-Asp-Trp-5Clw-Hyp-Glu-leu-[Cys(2Lut)-Thp-Glu-Ile-Cys]-NH₂ (GIP-231, SEQ ID NO: 231) of the following formula

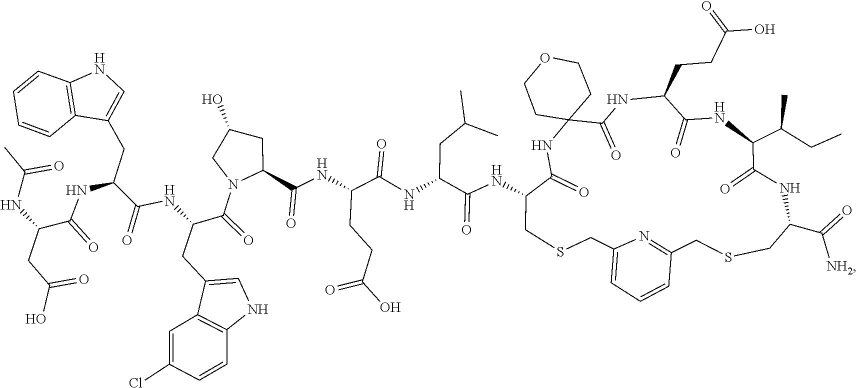 [SMILES: CC[C@H](C)[C@@H]1NC(=O)[C@H](CCC(=O)O)NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CC(=O)O)NC(C)=O)CSCc2cccc(n2)CSC[C@@H](C(N)=O)NC1=O]

compound Ac-Asp-Trp-5Clw-Hyp-Glu-leu-[Cys(2Lut)-Thp-Phe-Ile-Cys]-NH₂ (GIP-232, SEQ ID NO: 232) of the following formula

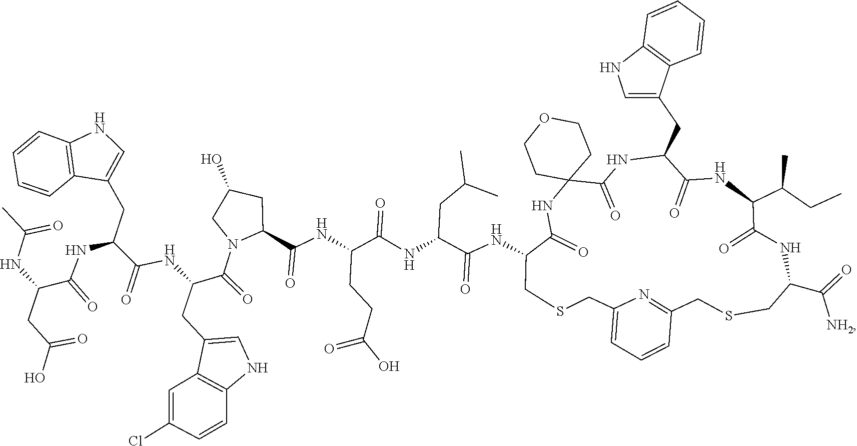 [SMILES: CC[C@H](C)[C@@H]1NC(=O)[C@H](Cc2c[nH]c3ccccc23)NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CC(=O)O)NC(C)=O)CSCc2cccc(n2)CSC[C@@H](C(N)=O)NC1=O]

compound Ac-Asp-Trp-5Clw-Hyp-Glu-leu-[Cys(2Lut)-Thp-Trp-Ile-Cys]-NH₂ (GIP-233, SEQ ID NO: 233) of the following formula

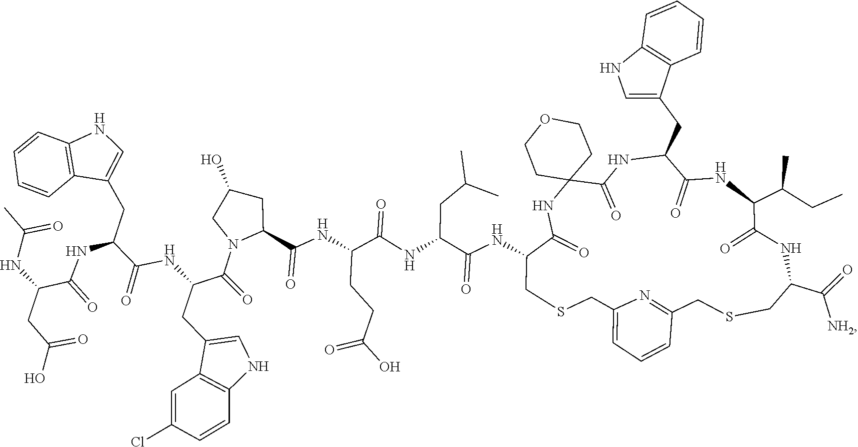 [SMILES: CC[C@H](C)[C@@H]1NC(=O)[C@H](Cc2c[nH]c3ccccc23)NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CC(=O)O)NC(C)=O)CSCc2cccc(n2)CSC[C@@H](C(N)=O)NC1=O]

compound Ac-Asp-Trp-5Clw-Hyp-Glu-leu-[Cys(2Lut)-Trp-Leu-Ile-Cys]-NH₂ (GIP-234, SEQ ID NO: 234) of the following formula

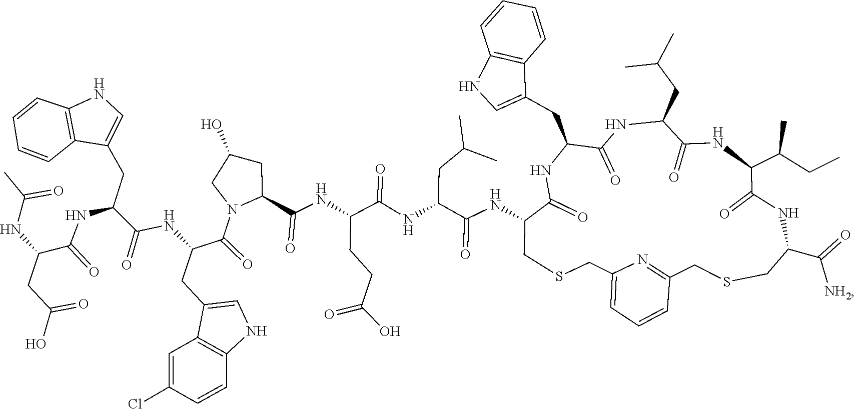 [SMILES: CC[C@H](C)[C@@H]1NC(=O)[C@H](CC(C)C)NC(=O)[C@H](Cc2c[nH]c3ccccc23)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CC(=O)O)NC(C)=O)CSCc2cccc(n2)CSC[C@@H](C(N)=O)NC1=O]

compound Ac-Asp-Trp-5Clw-Hyp-Glu-leu-[Cys(2Lut)-Glu-Leu-Ile-Cys]-NH₂ (GIP-235, SEQ ID NO: 235) of the following formula

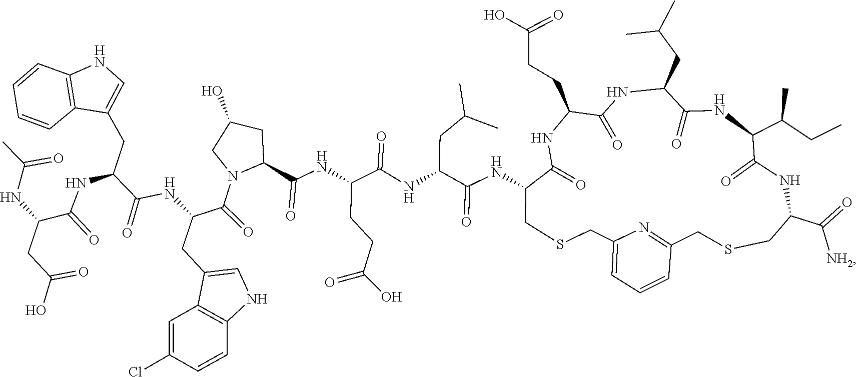 [SMILES: CC[C@H](C)[C@@H]1NC(=O)[C@H](CC(C)C)NC(=O)[C@H](CCC(=O)O)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CC(=O)O)NC(C)=O)CSCc2cccc(n2)CSC[C@@H](C(N)=O)NC1=O]

compound Ac-Asp-Trp-5Clw-Hyp-Glu-leu-[Cys(2Lut)-Thp-Ser-Ile-Cys]-NH₂ (GIP-236, SEQ ID NO: 236) of the following formula

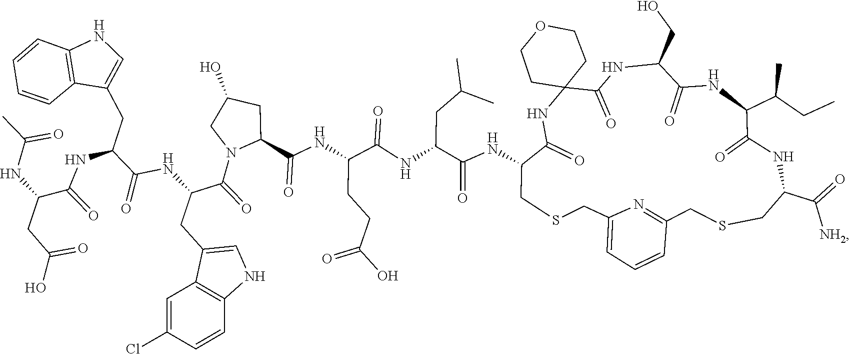 [SMILES: CC[C@H](C)[C@@H]1NC(=O)[C@H](CO)NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CC(=O)O)NC(C)=O)CSCc2cccc(n2)CSC[C@@H](C(N)=O)NC1=O]

compound Ac-Asp-Trp-5Clw-Hyp-Glu-leu-[Cys(2Lut)-Thp-Leu-Leu-Cys]-NH₂ (GIP-237, SEQ ID NO: 237) of the following formula

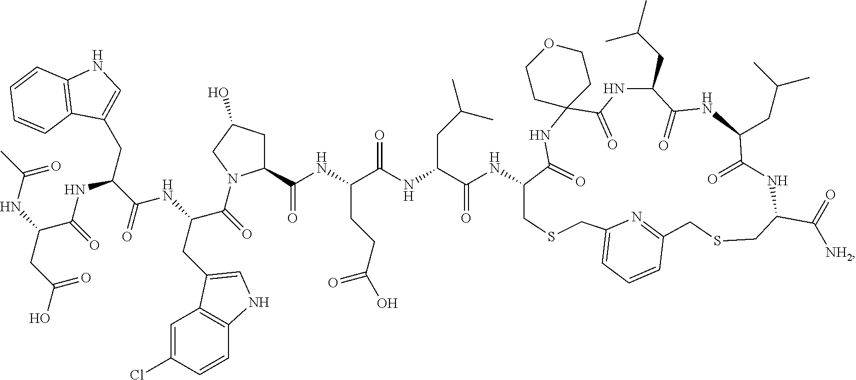 [SMILES: CC(=O)N[C@@H](CC(=O)O)C(=O)N[C@@H](Cc1c[nH]c2ccccc12)C(=O)N[C@@H](Cc1c[nH]c2ccc(Cl)cc12)C(=O)N1C[C@H](O)C[C@H]1C(=O)N[C@@H](CCC(=O)O)C(=O)N[C@H](CC(C)C)C(=O)N[C@H]1CSCc2cccc(n2)CSC[C@@H](C(N)=O)NC(=O)[C@H](CC(C)C)NC(=O)[C@H](CC(C)C)NC(=O)C2(CCOCC2)NC1=O]

compound Ac-Asp-Trp-5Clw-Hyp-Glu-leu-[Cys(2Lut)-Thp-Leu-Tle-Cys]-NH₂ (GIP-238, SEQ ID NO: 238) of the following formula

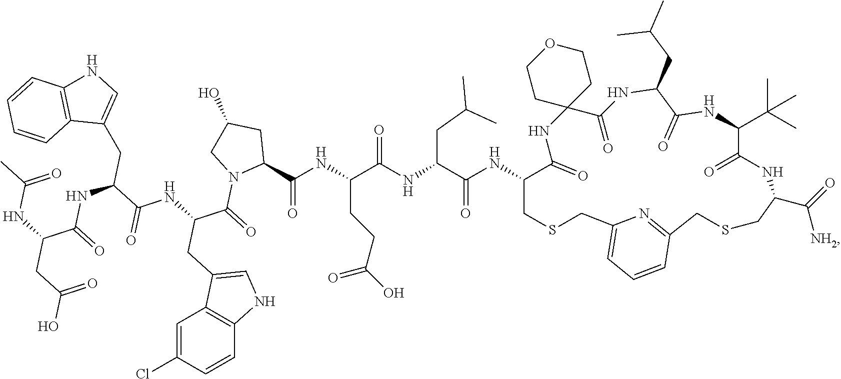 [SMILES: CC(=O)N[C@@H](CC(=O)O)C(=O)N[C@@H](Cc1c[nH]c2ccccc12)C(=O)N[C@@H](Cc1c[nH]c2ccc(Cl)cc12)C(=O)N1C[C@H](O)C[C@H]1C(=O)N[C@@H](CCC(=O)O)C(=O)N[C@H](CC(C)C)C(=O)N[C@H]1CSCc2cccc(n2)CSC[C@@H](C(N)=O)NC(=O)[C@H](C(C)(C)C)NC(=O)[C@H](CC(C)C)NC(=O)C2(CCOCC2)NC1=O]

compound Ac-Asp-Trp-5Clw-Tap-Glu-leu-[Cys(2Lut)-Thp-Leu-Ile-Cys]-NH₂ (GIP-239, SEQ ID NO: 239) of the following formula

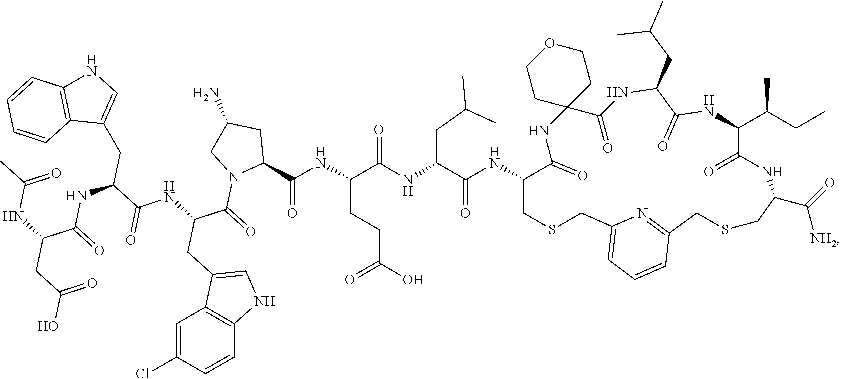 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](N)CN2C(=O)[C@H](Cc2c[nH]c3ccc(Cl)cc23)NC(=O)[C@H](Cc2c[nH]c3ccccc23)NC(=O)[C@H](CC(=O)O)NC(C)=O)CSCc2cccc(n2)CSC[C@@H](C(N)=O)NC1=O]

compound Ac-Tyr-Asp-Trp-Trp-Pro-Glu-Leu-[Cys(3MeBn)-Ala-Leu-Ile-Cys]-NH₂ (GIP-240, SEQ ID NO: 240) of the following formula

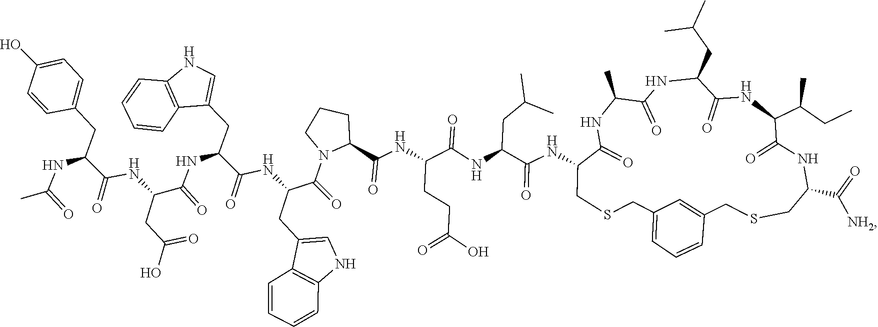 [SMILES: CC[C@H](C)[C@@H]1NC(=O)[C@H](CC(C)C)NC(=O)[C@H](C)NC(=O)[C@@H](NC(=O)[C@H](CC(C)C)NC(=O)[C@H](CCC(=O)O)NC(=O)[C@@H]2CCCN2C(=O)[C@H](Cc2c[nH]c3ccccc23)NC(=O)[C@H](Cc2c[nH]c3ccccc23)NC(=O)[C@H](CC(=O)O)NC(=O)[C@H](Cc2ccc(O)cc2)NC(C)=O)CSCc2cccc(c2)CSC[C@@H](C(N)=O)NC1=O]

compound H-Met-Tyr-Asp-Trp-Trp-Pro-Glu-Leu-[Cys(3MeBn)-Gln-Leu-Ile-Cys]-NH₂ (GIP-241, SEQ ID NO: 241) of the following formula

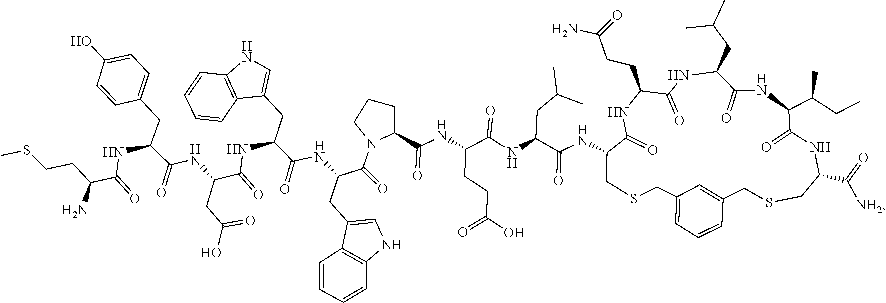 [SMILES: CC[C@H](C)[C@@H]1NC(=O)[C@H](CC(C)C)NC(=O)[C@H](CCC(N)=O)NC(=O)[C@@H](NC(=O)[C@H](CC(C)C)NC(=O)[C@H](CCC(=O)O)NC(=O)[C@@H]2CCCN2C(=O)[C@H](Cc2c[nH]c3ccccc23)NC(=O)[C@H](Cc2c[nH]c3ccccc23)NC(=O)[C@H](CC(=O)O)NC(=O)[C@H](Cc2ccc(O)cc2)NC(=O)[C@@H](N)CCSC)CSCc2cccc(c2)CSC[C@@H](C(N)=O)NC1=O]

compound H-Met-Tyr-Asp-Trp-Trp-Pro-Glu-Leu-[Cys(3MeBn)-Thr-Leu-Ile-Cys]-NH₂ (GIP-242, SEQ ID NO: 242) of the following formula

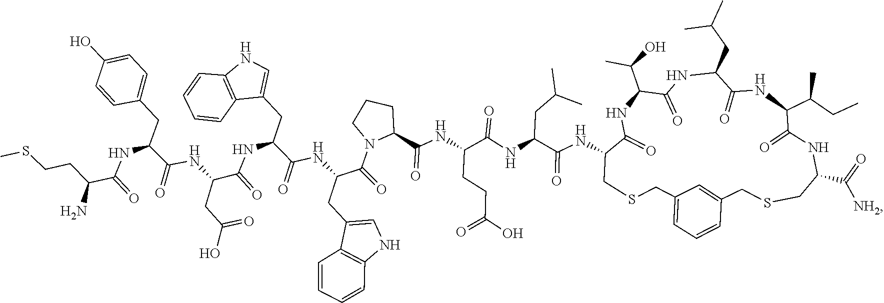 [SMILES: CC[C@H](C)[C@@H]1NC(=O)[C@H](CC(C)C)NC(=O)[C@H]([C@@H](C)O)NC(=O)[C@@H](NC(=O)[C@H](CC(C)C)NC(=O)[C@H](CCC(=O)O)NC(=O)[C@@H]2CCCN2C(=O)[C@H](Cc2c[nH]c3ccccc23)NC(=O)[C@H](Cc2c[nH]c3ccccc23)NC(=O)[C@H](CC(=O)O)NC(=O)[C@H](Cc2ccc(O)cc2)NC(=O)[C@@H](N)CCSC)CSCc2cccc(c2)CSC[C@@H](C(N)=O)NC1=O]

compound H-Met-Tyr-Asp-Trp-Trp-Pro-Glu-Leu-[Cys(3MeBn)-Lys-Leu-Ile-Cys]-NH₂ (GIP-243, SEQ ID NO: 243) of the following formula

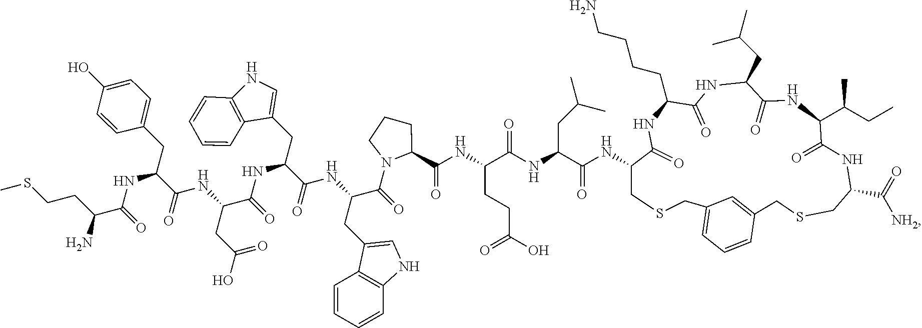 [SMILES: CC[C@H](C)[C@@H]1NC(=O)[C@H](CC(C)C)NC(=O)[C@H](CCCCN)NC(=O)[C@@H](NC(=O)[C@H](CC(C)C)NC(=O)[C@H](CCC(=O)O)NC(=O)[C@@H]2CCCN2C(=O)[C@H](Cc2c[nH]c3ccccc23)NC(=O)[C@H](Cc2c[nH]c3ccccc23)NC(=O)[C@H](CC(=O)O)NC(=O)[C@H](Cc2ccc(O)cc2)NC(=O)[C@@H](N)CCSC)CSCc2cccc(c2)CSC[C@@H](C(N)=O)NC1=O]

compound Ac-Asp-Trp-Trp-Pro-Glu-Leu-[Cys(3MeBn)-Ala-Leu-Ile-Cys]-NH₂ (GIP-244, SEQ ID NO: 244) of the following formula

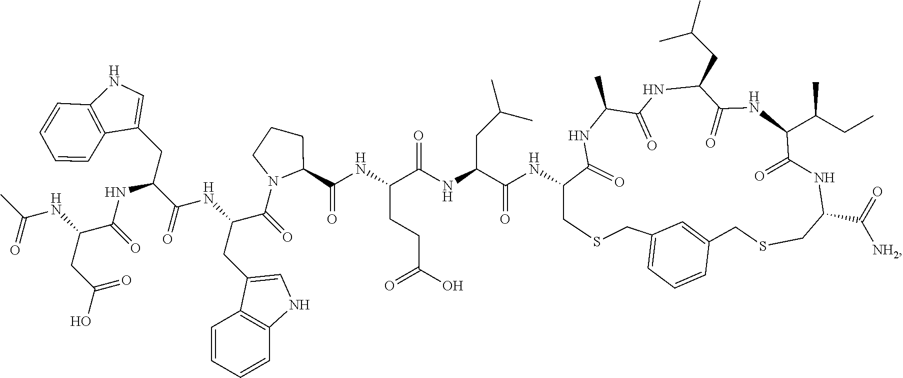 [SMILES: CC[C@H](C)[C@@H]1NC(=O)[C@H](CC(C)C)NC(=O)[C@H](C)NC(=O)[C@@H](NC(=O)[C@H](CC(C)C)NC(=O)[C@H](CCC(=O)O)NC(=O)[C@@H]2CCCN2C(=O)[C@H](Cc2c[nH]c3ccccc23)NC(=O)[C@H](Cc2c[nH]c3ccccc23)NC(=O)[C@H](CC(=O)O)NC(C)=O)CSCc2cccc(c2)CSC[C@@H](C(N)=O)NC1=O]

compound H-Met-Tyr-Asp-Trp-Trp-Pro-glu-Leu-[Cys(3MeBn)-Ala-Leu-Ile-Cys]-NH₂ (GIP-245, SEQ ID NO: 245) of the following formula

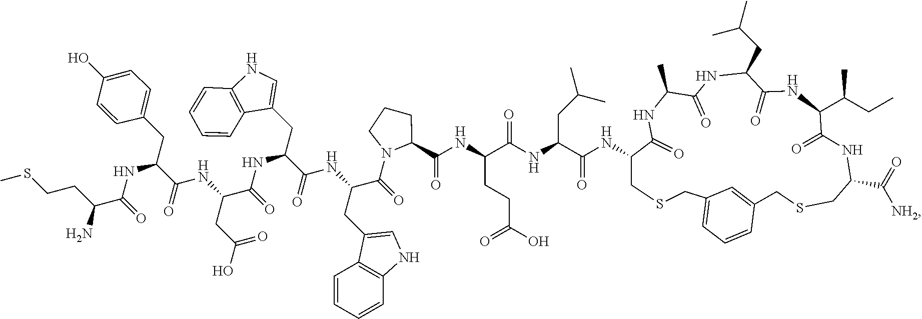 [SMILES: CC[C@H](C)[C@@H]1NC(=O)[C@H](CC(C)C)NC(=O)[C@H](C)NC(=O)[C@@H](NC(=O)[C@H](CC(C)C)NC(=O)[C@@H](CCC(=O)O)NC(=O)[C@@H]2CCCN2C(=O)[C@H](Cc2c[nH]c3ccccc23)NC(=O)[C@H](Cc2c[nH]c3ccccc23)NC(=O)[C@H](CC(=O)O)NC(=O)[C@H](Cc2ccc(O)cc2)NC(=O)[C@@H](N)CCSC)CSCc2cccc(c2)CSC[C@@H](C(N)=O)NC1=O]

compound H-Met-tyr-Asp-Trp-Trp-Pro-Glu-Leu-[Cys(3MeBn)-Ala-Leu-Ile-Cys]-NH₂ (GIP-246, SEQ ID NO: 246) of the following formula

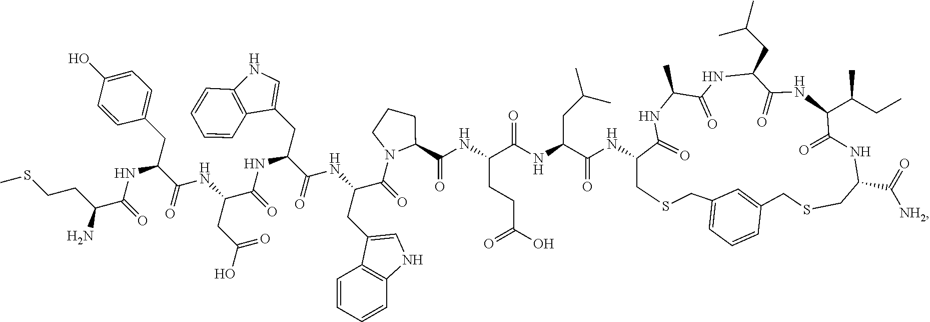

compound H-Met-Tyr-Asp-Trp-Trp-Pro-Glu-leu-[Cys(3MeBn)-Ala-Leu-Ile-Cys]-NH₂ (GIP-247, SEQ ID NO: 247) of the following formula

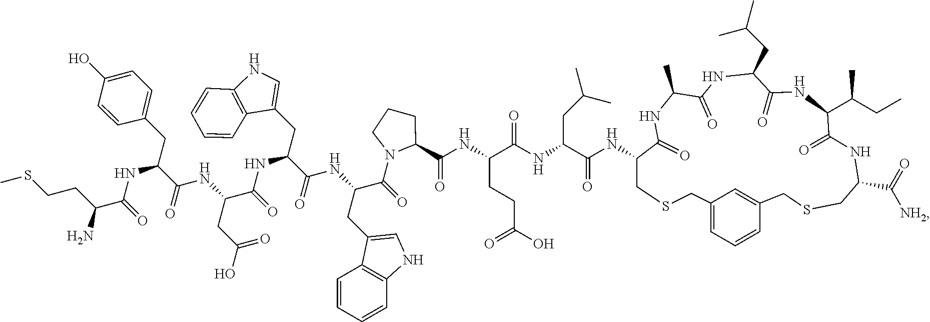

compound H-Met-Tyr-Asp-Trp-Trp-Pro-Glu-Leu-[Cys(3MeBn)-ala-Leu-Ile-Cys]-NH₂ (GIP-248, SEQ ID NO: 248) of the following formula

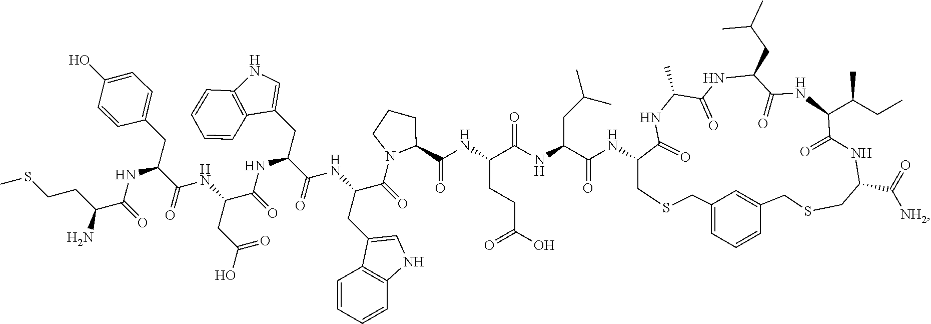 [SMILES: CC[C@H](C)[C@@H]1NC(=O)[C@H](CC(C)C)NC(=O)[C@@H](C)NC(=O)[C@@H](NC(=O)[C@H](CC(C)C)NC(=O)[C@H](CCC(=O)O)NC(=O)[C@@H]2CCCN2C(=O)[C@H](Cc2c[nH]c3ccccc23)NC(=O)[C@H](Cc2c[nH]c3ccccc23)NC(=O)[C@H](CC(=O)O)NC(=O)[C@H](Cc2ccc(O)cc2)NC(=O)[C@@H](N)CCSC)CSCc2cccc(c2)CSC[C@@H](C(N)=O)NC1=O]

compound H-Met-Ala-Asp-Trp-Trp-Pro-Glu-Leu-[Cys(3MeBn)-Ala-Leu-Ile-Cys]-NH₂ (GIP-249, SEQ ID NO: 249) of the following formula

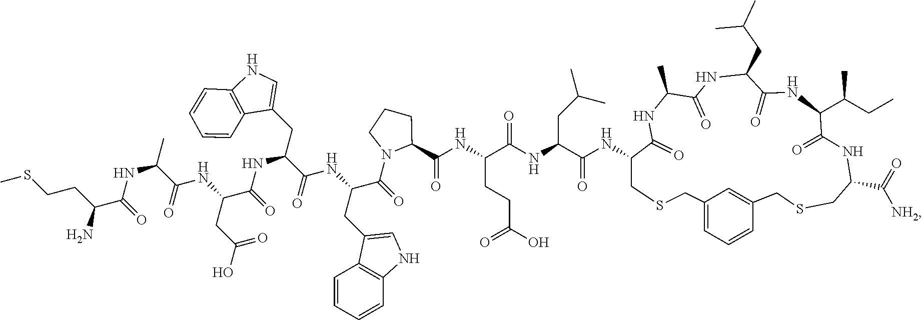 [SMILES: CC[C@H](C)[C@@H]1NC(=O)[C@H](CC(C)C)NC(=O)[C@H](C)NC(=O)[C@@H](NC(=O)[C@H](CC(C)C)NC(=O)[C@H](CCC(=O)O)NC(=O)[C@@H]2CCCN2C(=O)[C@H](Cc2c[nH]c3ccccc23)NC(=O)[C@H](Cc2c[nH]c3ccccc23)NC(=O)[C@H](CC(=O)O)NC(=O)[C@H](C)NC(=O)[C@@H](N)CCSC)CSCc2cccc(c2)CSC[C@@H](C(N)=O)NC1=O]

compound Ac-Asp-Trp-Trp-Pro-Glu-Leu-[Cys(3MeBn)-ala-Leu-Ile-AET] (GIP-250, SEQ ID NO: 250) of the following formula

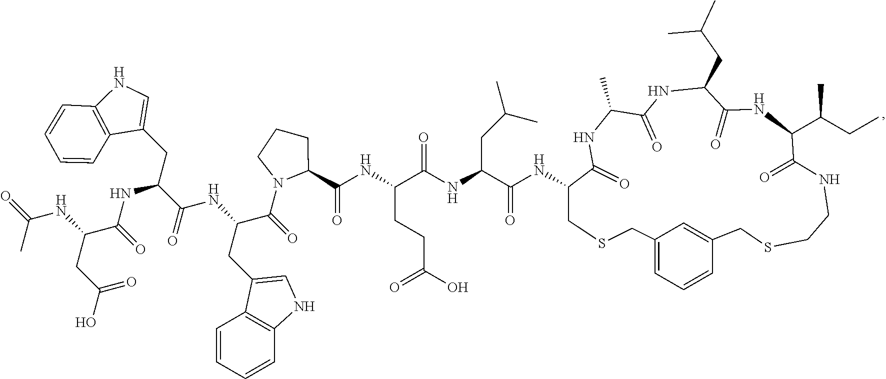 [SMILES: CC[C@H](C)[C@@H]1NC(=O)[C@H](CC(C)C)NC(=O)[C@@H](C)NC(=O)[C@@H](NC(=O)[C@H](CC(C)C)NC(=O)[C@H](CCC(=O)O)NC(=O)[C@@H]2CCCN2C(=O)[C@H](Cc2c[nH]c3ccccc23)NC(=O)[C@H](Cc2c[nH]c3ccccc23)NC(=O)[C@H](CC(=O)O)NC(C)=O)CSCc2cccc(c2)CSCCNC1=O]

compound Ac-Asp-Trp-Tip-Pro-Glu-Leu-[Cys(3MeBn)-Aib-Leu-Ile-AET] (GIP-251, SEQ ID NO: 251) of the following formula

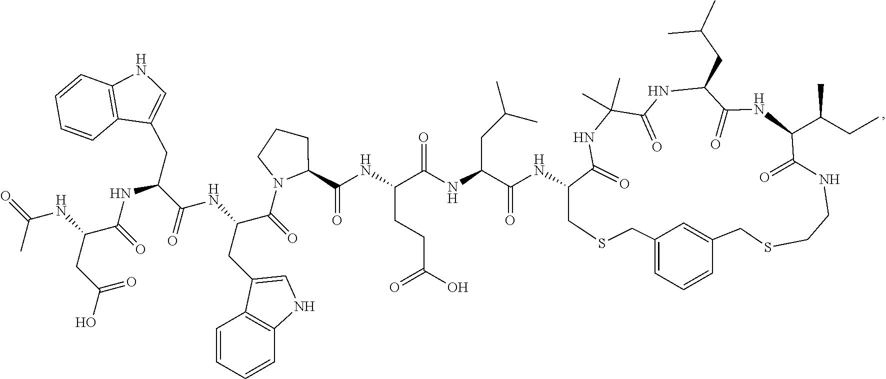 [SMILES: CC[C@H](C)[C@@H]1NC(=O)[C@H](CC(C)C)NC(=O)C(C)(C)NC(=O)[C@@H](NC(=O)[C@H](CC(C)C)NC(=O)[C@H](CCC(=O)O)NC(=O)[C@@H]2CCCN2C(=O)[C@H](Cc2c[nH]c3ccccc23)NC(=O)[C@H](Cc2c[nH]c3ccccc23)NC(=O)[C@H](CC(=O)O)NC(C)=O)CSCc2cccc(c2)CSCCNC1=O]

compound Ac-Asp-Trp-1Ni-Pip-Glu-Leu-[Cys(3MeBn)-Aib-Leu-Ile-Cys]-NH₂ (GIP-253, SEQ ID NO: 252) of the following formula

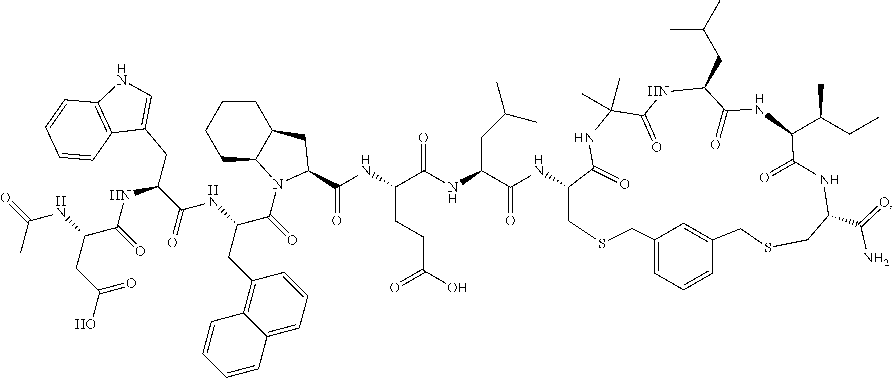 [SMILES: CC[C@H](C)[C@@H]1NC(=O)[C@H](CC(C)C)NC(=O)C(C)(C)NC(=O)[C@@H](NC(=O)[C@H](CC(C)C)NC(=O)[C@H](CCC(=O)O)NC(=O)[C@@H]2C[C@@H]3CCCC[C@@H]3N2C(=O)[C@H](Cc2cccc3ccccc23)NC(=O)[C@H](Cc2c[nH]c3ccccc23)NC(=O)[C@H](CC(=O)O)NC(C)=O)CSCc2cccc(c2)CSC[C@@H](C(N)=O)NC1=O]

compound Ac-Asp-Trp-1Ni-Pip-Glu-Leu-[Cys(3MeBn)-Aib-Leu-Ile-Cys]-NH₂ (GIP-253, SEQ ID NO: 253) of the following formula

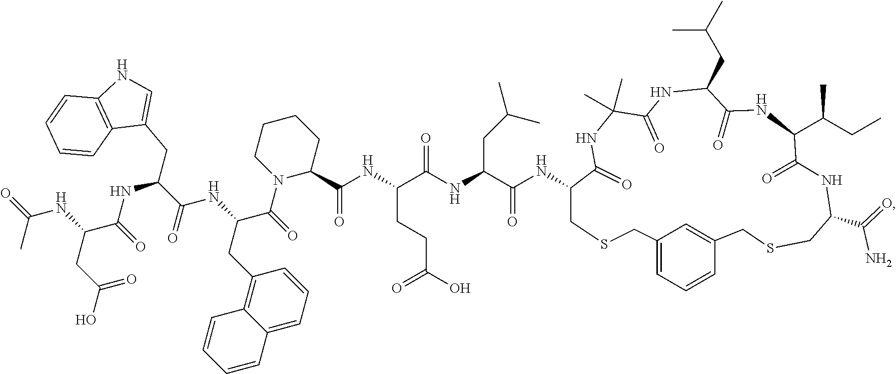 [SMILES: CC[C@H](C)[C@@H]1NC(=O)[C@H](CC(C)C)NC(=O)C(C)(C)NC(=O)[C@@H](NC(=O)[C@H](CC(C)C)NC(=O)[C@H](CCC(=O)O)NC(=O)[C@@H]2CCCCN2C(=O)[C@H](Cc2cccc3ccccc23)NC(=O)[C@H](Cc2c[nH]c3ccccc23)NC(=O)[C@H](CC(=O)O)NC(C)=O)CSCc2cccc(c2)CSC[C@@H](C(N)=O)NC1=O]

compound H-Met-Tyr-Asp-Trp-Trp-Pro-Glu-Leu-[Cys(3MeBn)-Ala-Leu-Ile-Cys]-NH₂ (GIP-254, SEQ ID NO: 254) of the following formula

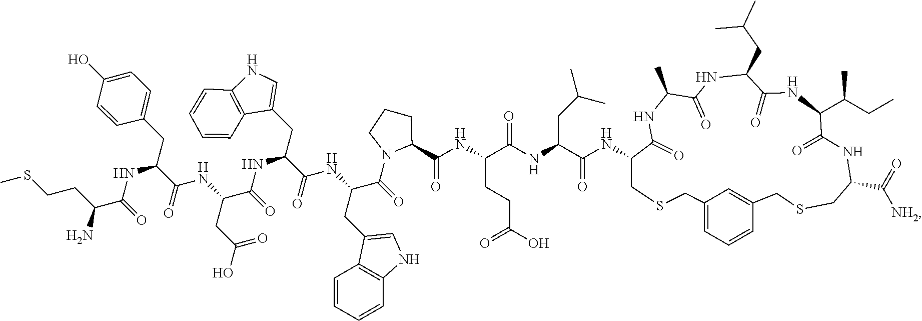 [SMILES: CC[C@H](C)[C@@H]1NC(=O)[C@H](CC(C)C)NC(=O)[C@H](C)NC(=O)[C@@H](NC(=O)[C@H](CC(C)C)NC(=O)[C@H](CCC(=O)O)NC(=O)[C@@H]2CCCN2C(=O)[C@H](Cc2c[nH]c3ccccc23)NC(=O)[C@H](Cc2c[nH]c3ccccc23)NC(=O)[C@H](CC(=O)O)NC(=O)[C@H](Cc2ccc(O)cc2)NC(=O)[C@@H](N)CCSC)CSCc2cccc(c2)CSC[C@@H](C(N)=O)NC1=O]

compound HO-Succinyl-Trp-Trp-Pro-Glu-Leu-[Cys(3MeBn)-Ala-Leu-Ile-Cys]-NH₂ (GIP-255, SEQ ID NO: 255) of the following formula

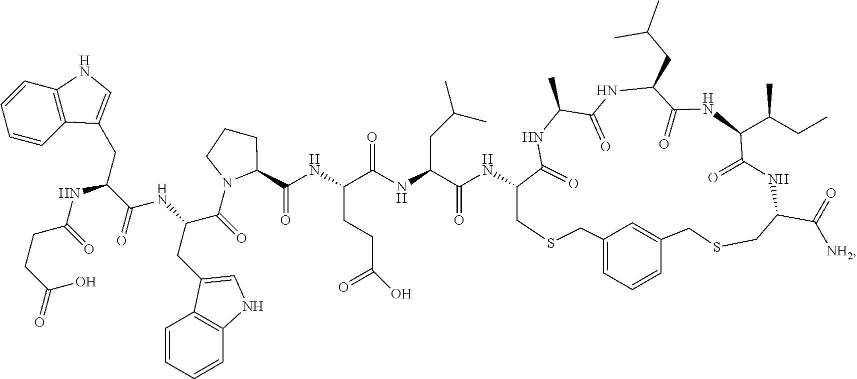 [SMILES: CC[C@H](C)[C@@H]1NC(=O)[C@H](CC(C)C)NC(=O)[C@H](C)NC(=O)[C@@H](NC(=O)[C@H](CC(C)C)NC(=O)[C@H](CCC(=O)O)NC(=O)[C@@H]2CCCN2C(=O)[C@H](Cc2c[nH]c3ccccc23)NC(=O)[C@H](Cc2c[nH]c3ccccc23)NC(=O)CCC(=O)O)CSCc2cccc(c2)CSC[C@@H](C(N)=O)NC1=O]

compound HO-Succinyl-Trp-Trp-Pro-Glu-Leu-[Cys(3MeBn)-Ala-Leu-Ile-AET] (GIP-256, SEQ ID NO: 256) of the following formula

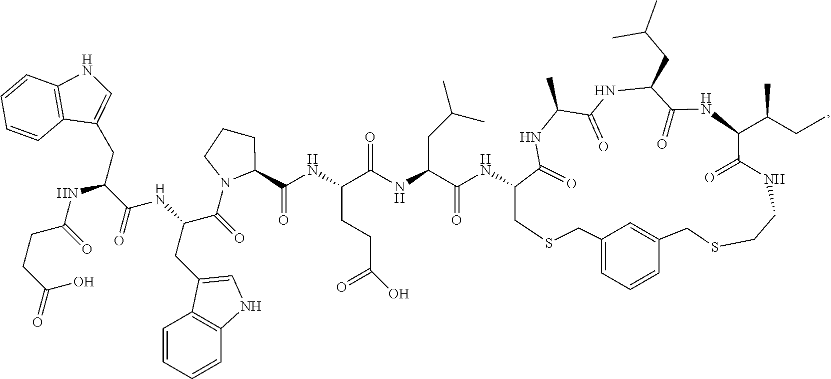

compound HO-Succinyl-Trp-Trp-Pro-Glu-Leu-[Cys(2Lut)-Ala-Leu-Ile-Cys]-NH₂ (GIP-257, SEQ ID NO: 257) of the following formula

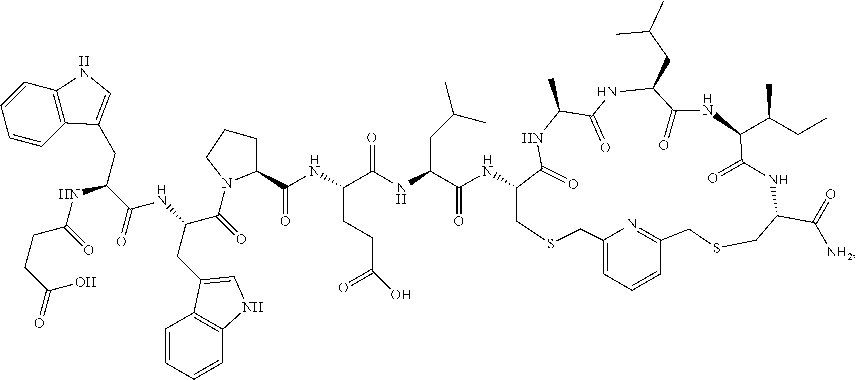 [SMILES: CC[C@H](C)[C@@H]1NC(=O)[C@H](CC(C)C)NC(=O)[C@H](C)NC(=O)[C@@H](NC(=O)[C@H](CC(C)C)NC(=O)[C@H](CCC(=O)O)NC(=O)[C@@H]2CCCN2C(=O)[C@H](Cc2c[nH]c3ccccc23)NC(=O)[C@H](Cc2c[nH]c3ccccc23)NC(=O)CCC(=O)O)CSCc2cccc(n2)CSC[C@@H](C(N)=O)NC1=O]

compound HO-Succinyl-Trp-Trp-Pro-Glu-leu-[Cys(3MeBn)-Aib-Leu-Ile-Cys]-NH₂ (GIP-258, SEQ ID NO: 258) of the following formula

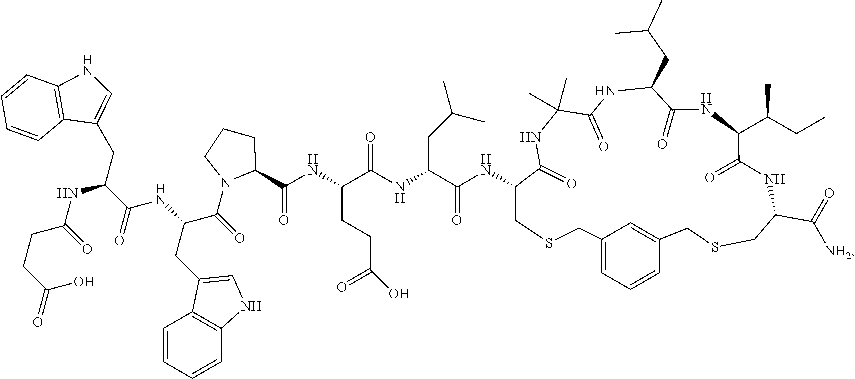 [SMILES: CC[C@H](C)[C@@H]1NC(=O)[C@H](CC(C)C)NC(=O)C(C)(C)NC(=O)[C@@H](NC(=O)[C@@H](CC(C)C)NC(=O)[C@H](CCC(=O)O)NC(=O)[C@@H]2CCCN2C(=O)[C@H](Cc2c[nH]c3ccccc23)NC(=O)[C@H](Cc2c[nH]c3ccccc23)NC(=O)CCC(=O)O)CSCc2cccc(c2)CSC[C@@H](C(N)=O)NC1=O]

compound HO-Succinyl-Trp-Trp-Pro-Glu-Leu-[Cys(3MeBn)-Aib-Leu-Ile-Cys]-NH₂ (GIP-259, SEQ ID NO: 259) of the following formula

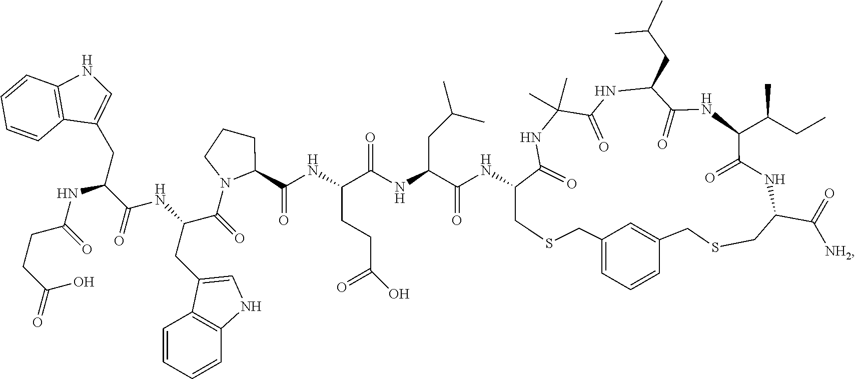 [SMILES: CC[C@H](C)[C@@H]1NC(=O)[C@H](CC(C)C)NC(=O)C(C)(C)NC(=O)[C@@H](NC(=O)[C@H](CC(C)C)NC(=O)[C@H](CCC(=O)O)NC(=O)[C@@H]2CCCN2C(=O)[C@H](Cc2c[nH]c3ccccc23)NC(=O)[C@H](Cc2c[nH]c3ccccc23)NC(=O)CCC(=O)O)CSCc2cccc(c2)CSC[C@@H](C(N)=O)NC1=O]

compound HO-Succinyl-Trp-Trp-Pro-Glu-Leu-[Cys(3MeBn)-Aib-Hle-Ile-Cys]-NH₂ (GIP-260, SEQ ID NO: 260) of the following formula

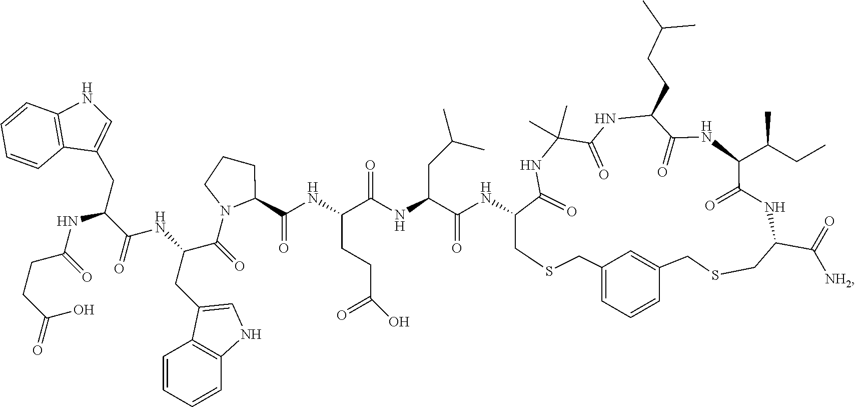 [SMILES: CC[C@H](C)[C@@H]1NC(=O)[C@H](CCC(C)C)NC(=O)C(C)(C)NC(=O)[C@@H](NC(=O)[C@H](CC(C)C)NC(=O)[C@H](CCC(=O)O)NC(=O)[C@@H]2CCCN2C(=O)[C@H](Cc2c[nH]c3ccccc23)NC(=O)[C@H](Cc2c[nH]c3ccccc23)NC(=O)CCC(=O)O)CSCc2cccc(c2)CSC[C@@H](C(N)=O)NC1=O]

compound HO-Succinyl-Trp-Trp-Pro-Glu-Leu-[Cys(3MeBn)-Thp-Leu-Ile-Cys]-NH₂ (GIP-261, SEQ ID NO: 261) of the following formula

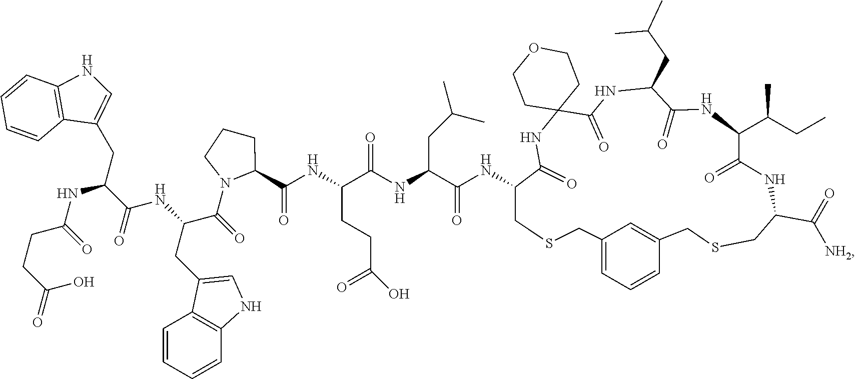 [SMILES: CC[C@H](C)[C@@H]1NC(=O)[C@H](CC(C)C)NC(=O)C2(CCOCC2)NC(=O)[C@@H](NC(=O)[C@H](CC(C)C)NC(=O)[C@H](CCC(=O)O)NC(=O)[C@@H]2CCCN2C(=O)[C@H](Cc2c[nH]c3ccccc23)NC(=O)[C@H](Cc2c[nH]c3ccccc23)NC(=O)CCC(=O)O)CSCc2cccc(c2)CSC[C@@H](C(N)=O)NC1=O]

compound Ac-Asp-Ocf-5Clw-Hyp-Glu-leu-[Cys(2Lut)-Thp-Leu-Ile-Cys]-NH₂ (GIP-262, SEQ ID NO: 262) of the following formula

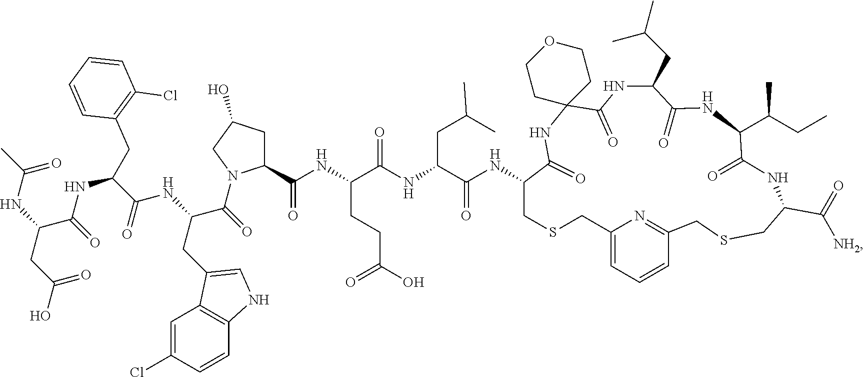 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2ccccc2Cl)NC(=O)[C@H](CC(=O)O)NC(C)=O)CSCc2cccc(n2)CSC[C@@H](C(N)=O)NC1=O]

compound Ac-Asp-Egn-5Clw-Hyp-Glu-leu-[Cys(2Lut)-Thp-Leu-Ile-Cys]-NH₂ (GIP-263, SEQ ID NO: 263) of the following formula

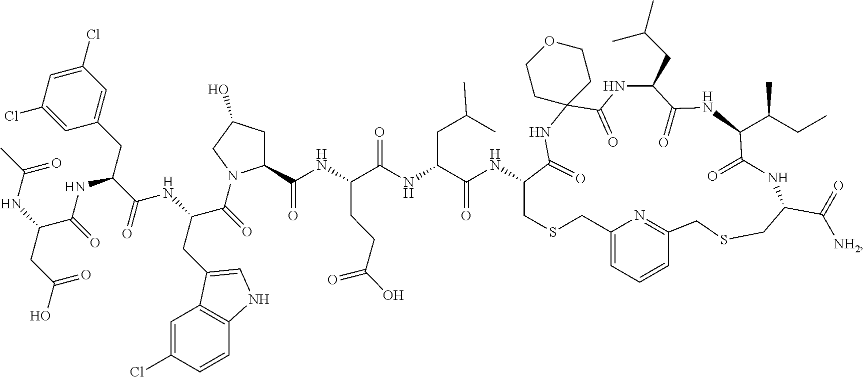 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2cc(Cl)cc(Cl)c2)NC(=O)[C@H](CC(=O)O)NC(C)=O)CSCc2cccc(n2)CSC[C@@H](C(N)=O)NC1=O]

compound Ac-Asp-Trp-Egp-Hyp-Glu-leu-[Cys(2Lut)-Thp-Leu-Ile-Cys]-NH₂ (GIP-265, SEQ ID NO: 264) of the following formula

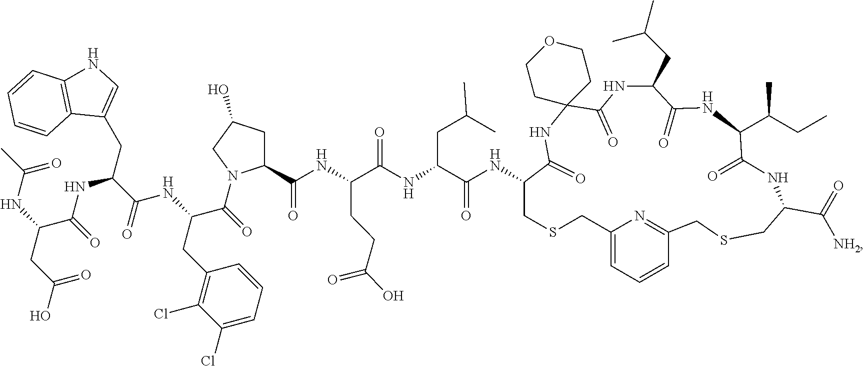 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2cccc(Cl)c2Cl)NC(=O)[C@H](Cc2c[nH]c3ccccc23)NC(=O)[C@H](CC(=O)O)NC(C)=O)CSCc2cccc(n2)CSC[C@@H](C(N)=O)NC1=O]

compound Ac-Asp-Trp-5Clw-Hyp-Glu-leu-[Cys(2Lut)-Thp-Leu-Phe-Cys]-NH₂ (GIP-266, SEQ ID NO: 265) of the following formula

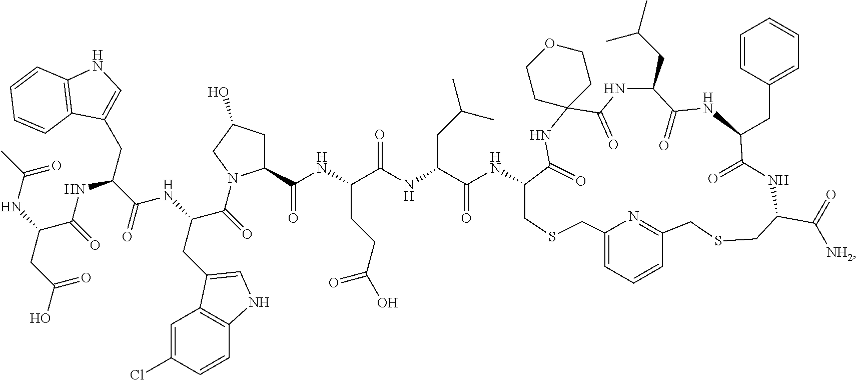 [SMILES: CC(=O)N[C@@H](CC(=O)O)C(=O)N[C@@H](Cc1c[nH]c2ccccc12)C(=O)N[C@@H](Cc1c[nH]c2ccc(Cl)cc12)C(=O)N1C[C@H](O)C[C@H]1C(=O)N[C@@H](CCC(=O)O)C(=O)N[C@H](CC(C)C)C(=O)N[C@H]1CSCc2cccc(n2)CSC[C@@H](C(N)=O)NC(=O)[C@H](Cc2ccccc2)NC(=O)[C@H](CC(C)C)NC(=O)C2(CCOCC2)NC1=O]

compound Ac-Asp-Trp-7Nw-Hyp-Glu-leu-[Cys(2Lut)-Thp-Leu-Ile-Cys]-NH₂ (GIP-267, SEQ ID NO: 266) of the following formula

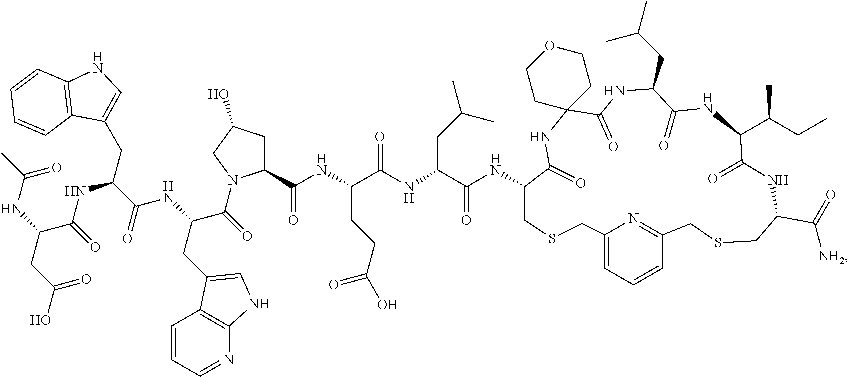 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ncccc23)NC(=O)[C@H](Cc2c[nH]c3ccccc23)NC(=O)[C@H](CC(=O)O)NC(C)=O)CSCc2cccc(n2)CSC[C@@H](C(N)=O)NC1=O]

compound DOTA-Ttds-Asp-Trp-Trp-Pro-glu-Leu-[Cys(3MeBn)-Aib-Leu-Ile-Cys]-NH₂ (GIP-270, SEQ ID NO: 267) of the following formula

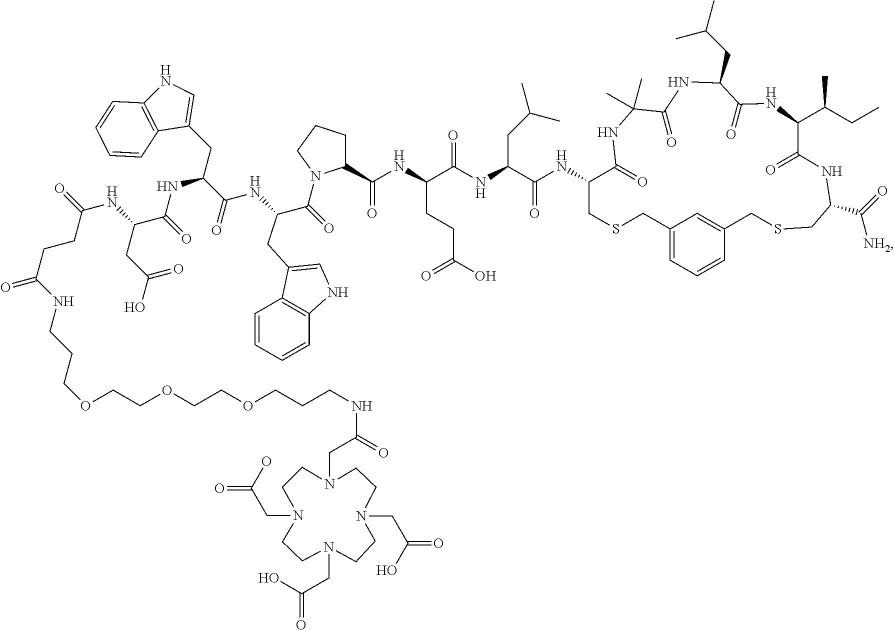

compound DOTA-Ttds-asp-Trp-Trp-Pro-Glu-Leu-[Cys(3MeBn)-ala-Leu-Ile-Cys]-NH₂ (GIP-271, SEQ ID NO: 268) of the following formula

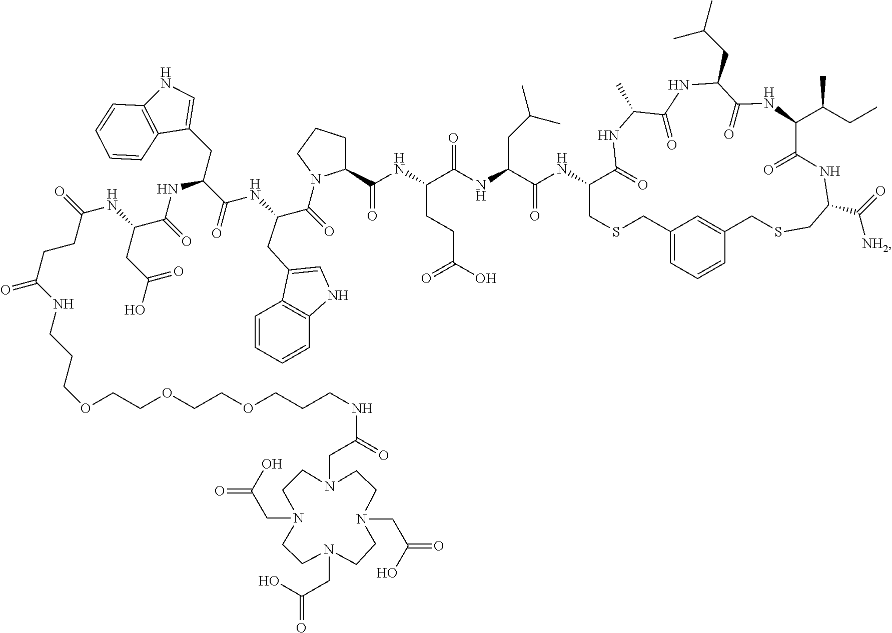 [SMILES: CC[C@H](C)[C@@H]1NC(=O)[C@H](CC(C)C)NC(=O)[C@@H](C)NC(=O)[C@@H](NC(=O)[C@H](CC(C)C)NC(=O)[C@H](CCC(=O)O)NC(=O)[C@@H]2CCCN2C(=O)[C@H](Cc2c[nH]c3ccccc23)NC(=O)[C@H](Cc2c[nH]c3ccccc23)NC(=O)[C@H](CC(=O)O)NC(=O)CCC(=O)NCCCOCCOCCOCCCNC(=O)CN2CCN(CC(=O)O)CCN(CC(=O)O)CCN(CC(=O)O)CC2)CSCc2cccc(c2)CSC[C@@H](C(N)=O)NC1=O]

compound Ac-Asp-Bta-Trp-Pro-Glu-Leu-[Cys(3MeBn)-Aib-Leu-Ile-Cys]-Ttds-Lys(DOTA)-NH₂ (GIP-273, SEQ ID NO: 269) of the following formula

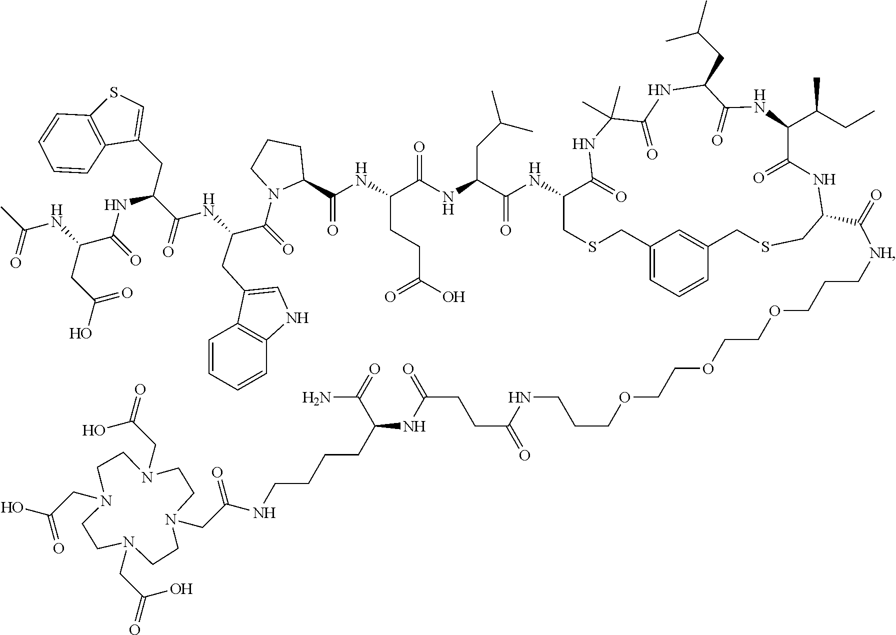 [SMILES: CC[C@H](C)[C@@H]1NC(=O)[C@H](CC(C)C)NC(=O)C(C)(C)NC(=O)[C@@H](NC(=O)[C@H](CC(C)C)NC(=O)[C@H](CCC(=O)O)NC(=O)[C@@H]2CCCN2C(=O)[C@H](Cc2c[nH]c3ccccc23)NC(=O)[C@H](Cc2csc3ccccc23)NC(=O)[C@H](CC(=O)O)NC(C)=O)CSCc2cccc(c2)CSC[C@@H](C(=O)NCCCOCCOCCOCCCNC(=O)CCC(=O)N[C@@H](CCCCNC(=O)CN2CCN(CC(=O)O)CCN(CC(=O)O)CCN(CC(=O)O)CC2)C(N)=O)NC1=O]

compound Ac-Asp-Trp-Trp-Pro-Nme-Leu-[Cys(3MeBn)-Aib-Leu-Ile-Cys]-Ttds-Lys(DOTA)-NH₂ (GIP-274, SEQ ID NO: 270) of the following formula

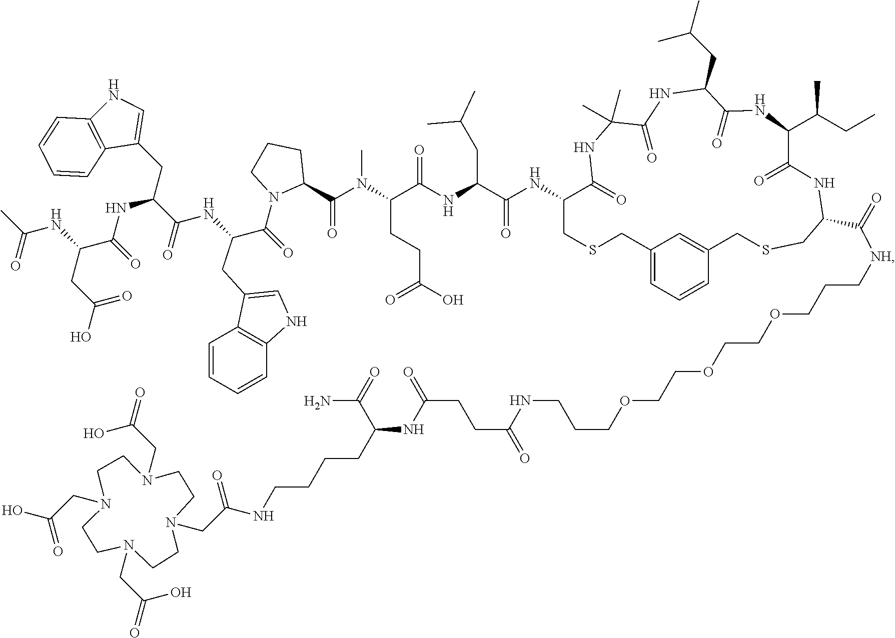 [SMILES: CC[C@H](C)[C@@H]1NC(=O)[C@H](CC(C)C)NC(=O)C(C)(C)NC(=O)[C@@H](NC(=O)[C@H](CC(C)C)NC(=O)[C@H](CCC(=O)O)N(C)C(=O)[C@@H]2CCCN2C(=O)[C@H](Cc2c[nH]c3ccccc23)NC(=O)[C@H](Cc2c[nH]c3ccccc23)NC(=O)[C@H](CC(=O)O)NC(C)=O)CSCc2cccc(c2)CSC[C@@H](C(=O)NCCCOCCOCCOCCCNC(=O)CCC(=O)N[C@@H](CCCCNC(=O)CN2CCN(CC(=O)O)CCN(CC(=O)O)CCN(CC(=O)O)CC2)C(N)=O)NC1=O]

compound Ac-Asp-Trp-Trp-Pro-Glu-Leu-[Cys(3MeBn)-Aib-Tle-Ile-Cys]-Ttds-Lys(DOTA)-NH₂ (GIP-276, SEQ ID NO: 271) of the following formula

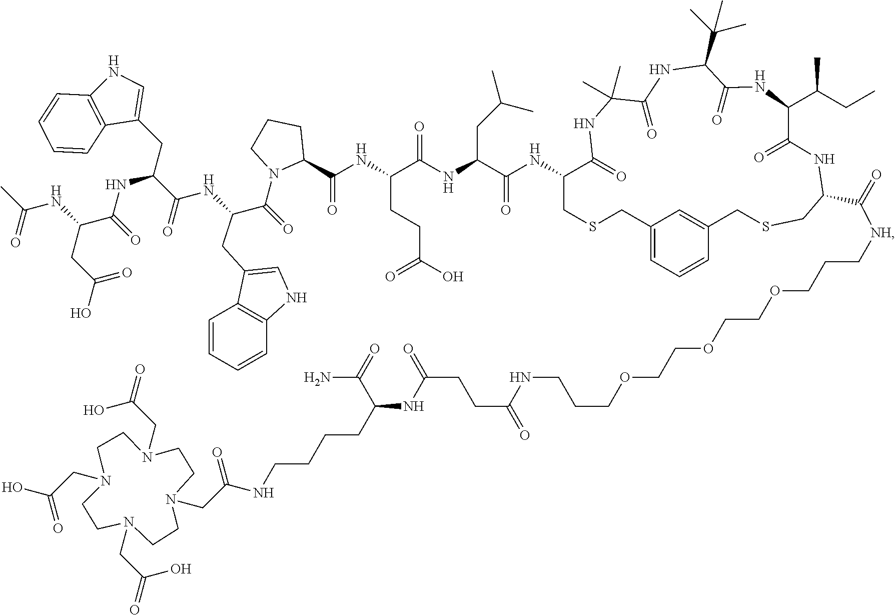 [SMILES: CC[C@H](C)[C@@H]1NC(=O)[C@H](C(C)(C)C)NC(=O)C(C)(C)NC(=O)[C@@H](NC(=O)[C@H](CC(C)C)NC(=O)[C@H](CCC(=O)O)NC(=O)[C@@H]2CCCN2C(=O)[C@H](Cc2c[nH]c3ccccc23)NC(=O)[C@H](Cc2c[nH]c3ccccc23)NC(=O)[C@H](CC(=O)O)NC(C)=O)CSCc2cccc(c2)CSC[C@@H](C(=O)NCCCOCCOCCOCCCNC(=O)CCC(=O)N[C@@H](CCCCNC(=O)CN2CCN(CC(=O)O)CCN(CC(=O)O)CCN(CC(=O)O)CC2)C(N)=O)NC1=O]

compound Ac-Asp-5Clw-Trp-Pro-Glu-Leu-[Cys(3MeBn)-Aib-Leu-Ile-Cys]-Ttds-Lys(DOTA)-NH₂ (GIP-277, SEQ ID NO: 272) of the following formula

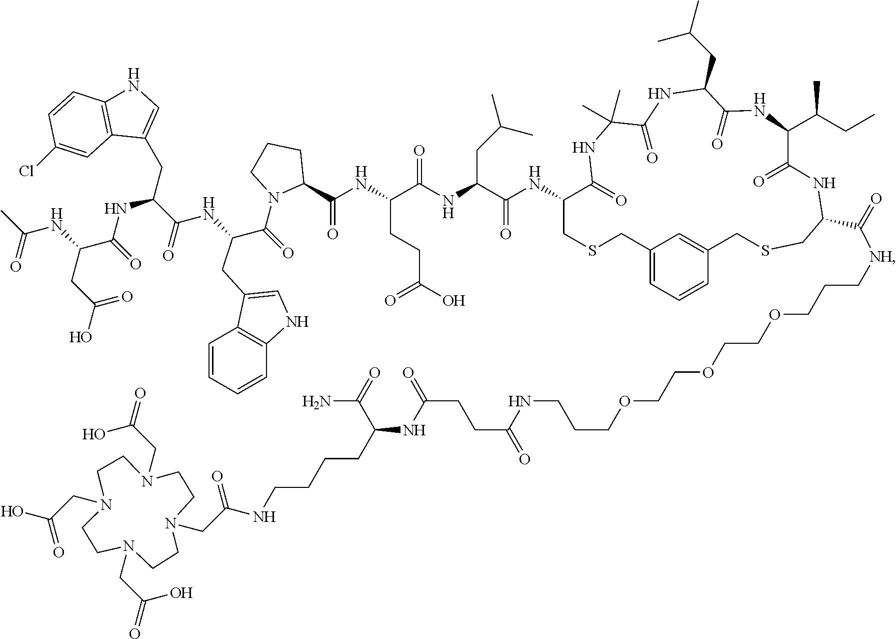 [SMILES: CC[C@H](C)[C@@H]1NC(=O)[C@H](CC(C)C)NC(=O)C(C)(C)NC(=O)[C@@H](NC(=O)[C@H](CC(C)C)NC(=O)[C@H](CCC(=O)O)NC(=O)[C@@H]2CCCN2C(=O)[C@H](Cc2c[nH]c3ccccc23)NC(=O)[C@H](Cc2c[nH]c3ccc(Cl)cc23)NC(=O)[C@H](CC(=O)O)NC(C)=O)CSCc2cccc(c2)CSC[C@@H](C(=O)NCCCOCCOCCOCCCNC(=O)CCC(=O)N[C@@H](CCCCNC(=O)CN2CCN(CC(=O)O)CCN(CC(=O)O)CCN(CC(=O)O)CC2)C(N)=O)NC1=O]

compound 4ClPhp-Asp-Trp-Trp-Pro-Glu-leu-[Cys(3MeBn)-Aib-Leu-Ile-Cys]-Ttds-Lys(DOTA)-NH₂ (GIP-279, SEQ ID NO: 273) of the following formula

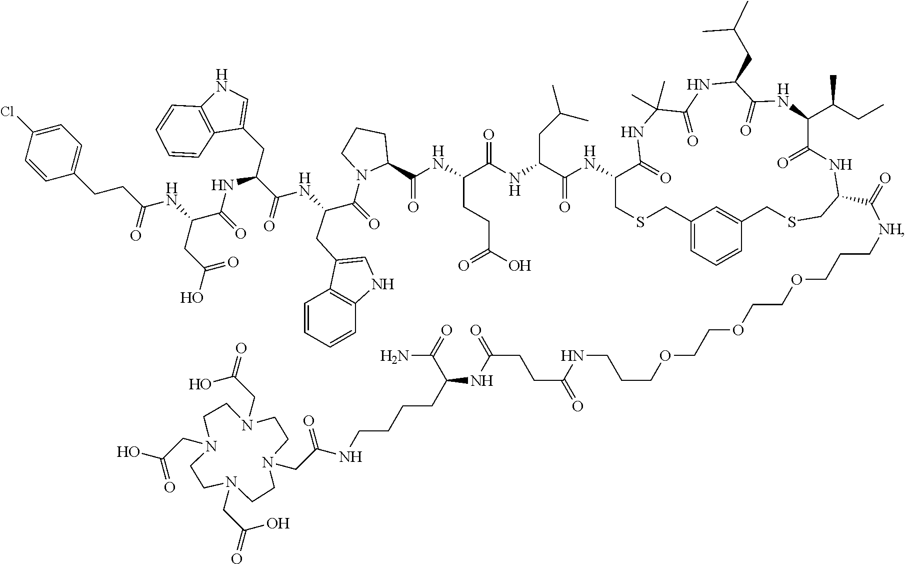 [SMILES: CC[C@H](C)[C@@H]1NC(=O)[C@H](CC(C)C)NC(=O)C(C)(C)NC(=O)[C@@H](NC(=O)[C@@H](CC(C)C)NC(=O)[C@H](CCC(=O)O)NC(=O)[C@@H]2CCCN2C(=O)[C@H](Cc2c[nH]c3ccccc23)NC(=O)[C@H](Cc2c[nH]c3ccccc23)NC(=O)[C@H](CC(=O)O)NC(=O)CCc2ccc(Cl)cc2)CSCc2cccc(c2)CSC[C@@H](C(=O)NCCCOCCOCCOCCCNC(=O)CCC(=O)N[C@@H](CCCCNC(=O)CN2CCN(CC(=O)O)CCN(CC(=O)O)CCN(CC(=O)O)CC2)C(N)=O)NC1=O]

compound Nph2p-Asp-Trp-Trp-Pro-Glu-leu-[Cys(3MeBn)-Aib-Leu-Ile-Cys]-Ttds-Lys(DOTA)-NH₂ (GIP-281, SEQ ID NO: 274) of the following formula

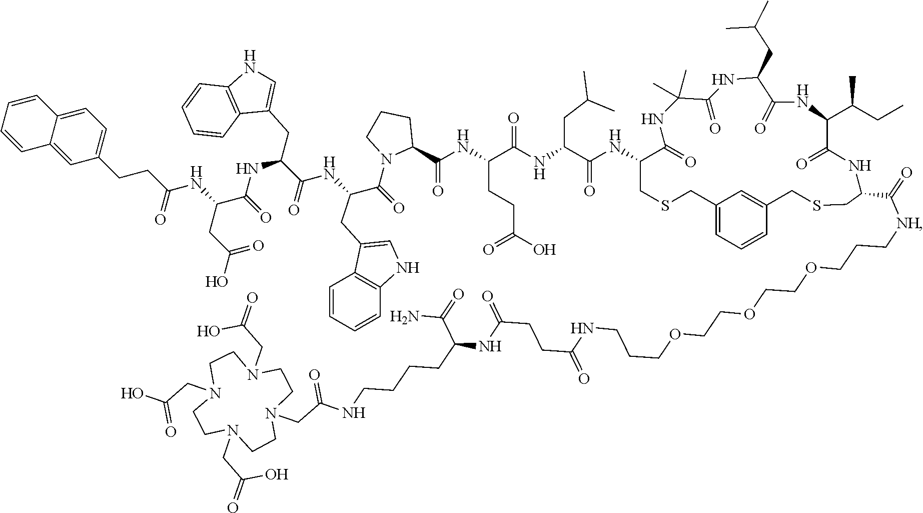 [SMILES: CC[C@H](C)[C@@H]1NC(=O)[C@H](CC(C)C)NC(=O)C(C)(C)NC(=O)[C@@H](NC(=O)[C@@H](CC(C)C)NC(=O)[C@H](CCC(=O)O)NC(=O)[C@@H]2CCCN2C(=O)[C@H](Cc2c[nH]c3ccccc23)NC(=O)[C@H](Cc2c[nH]c3ccccc23)NC(=O)[C@H](CC(=O)O)NC(=O)CCc2ccc3ccccc3c2)CSCc2cccc(c2)CSC[C@@H](C(=O)NCCCOCCOCCOCCCNC(=O)CCC(=O)N[C@@H](CCCCNC(=O)CN2CCN(CC(=O)O)CCN(CC(=O)O)CCN(CC(=O)O)CC2)C(N)=O)NC1=O]

compound Ac-Asp-Trp-6Clw-Pro-Glu-leu-[Cys(3MeBn)-Aib-Leu-Ile-Cys]-Ttds-Lys(DOTA)-NH₂ (GIP-282, SEQ D NO: 275) of the following formula

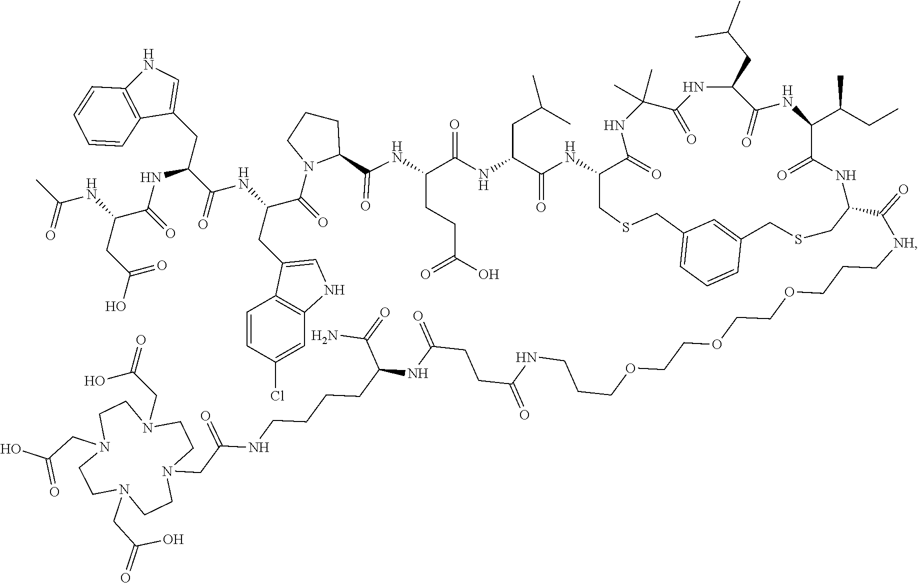 [SMILES: CC[C@H](C)[C@@H]1NC(=O)[C@H](CC(C)C)NC(=O)C(C)(C)NC(=O)[C@@H](NC(=O)[C@@H](CC(C)C)NC(=O)[C@H](CCC(=O)O)NC(=O)[C@@H]2CCCN2C(=O)[C@H](Cc2c[nH]c3cc(Cl)ccc23)NC(=O)[C@H](Cc2c[nH]c3ccccc23)NC(=O)[C@H](CC(=O)O)NC(C)=O)CSCc2cccc(c2)CSC[C@@H](C(=O)NCCCOCCOCCOCCCNC(=O)CCC(=O)N[C@@H](CCCCNC(=O)CN2CCN(CC(=O)O)CCN(CC(=O)O)CCN(CC(=O)O)CC2)C(N)=O)NC1=O]

compound DOTA-Ttds-Asp-6Fw-Trp-Pro-Glu-leu-[Cys(3MeBn)-Aib-Leu-Ile-AET] (GIP-283, SEQ ID NO: 276) of the following formula

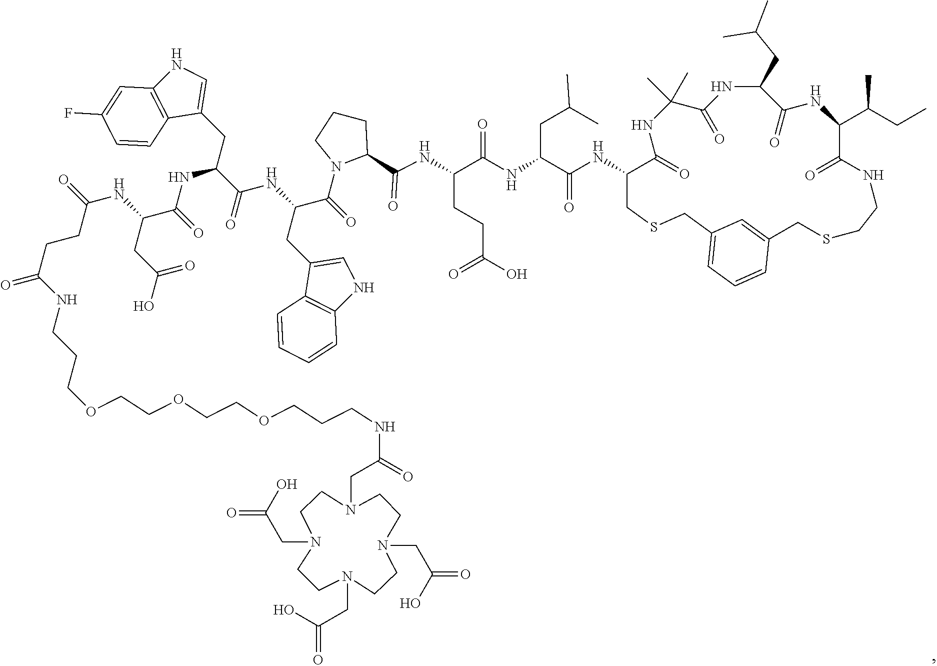 [SMILES: CC[C@H](C)[C@@H]1NC(=O)[C@H](CC(C)C)NC(=O)C(C)(C)NC(=O)[C@@H](NC(=O)[C@@H](CC(C)C)NC(=O)[C@H](CCC(=O)O)NC(=O)[C@@H]2CCCN2C(=O)[C@H](Cc2c[nH]c3ccccc23)NC(=O)[C@H](Cc2c[nH]c3cc(F)ccc23)NC(=O)[C@H](CC(=O)O)NC(=O)CCC(=O)NCCCOCCOCCOCCCNC(=O)CN2CCN(CC(=O)O)CCN(CC(=O)O)CCN(CC(=O)O)CC2)CSCc2cccc(c2)CSCCNC1=O]

compound Ac-Asp-Trp-1Ni-Pro-Glu-Hle-[Cys(3MeBn)-Aib-Leu-Ile-Cys]-NH₂ (GIP-315, SEQ ID NO: 277) of the following formula

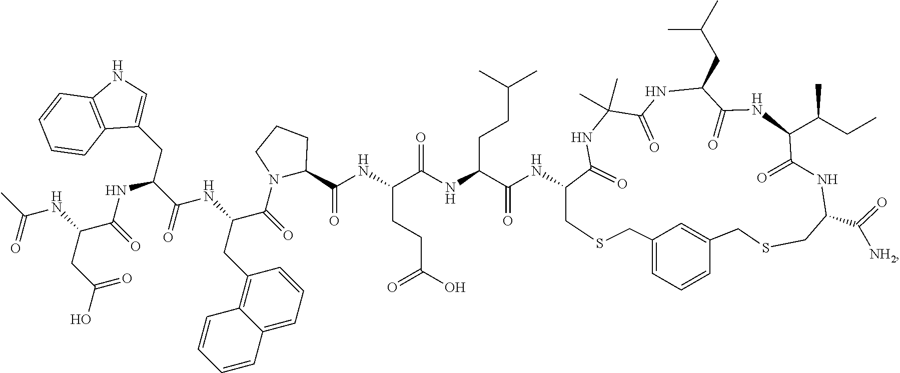 [SMILES: CC[C@H](C)[C@@H]1NC(=O)[C@H](CC(C)C)NC(=O)C(C)(C)NC(=O)[C@@H](NC(=O)[C@H](CCC(C)C)NC(=O)[C@H](CCC(=O)O)NC(=O)[C@@H]2CCCN2C(=O)[C@H](Cc2cccc3ccccc23)NC(=O)[C@H](Cc2c[nH]c3ccccc23)NC(=O)[C@H](CC(=O)O)NC(C)=O)CSCc2cccc(c2)CSC[C@@H](C(N)=O)NC1=O]

compound HO-Succinyl-Trp-Trp-Hyp-Glu-Leu-[Cys(3MeBn)-Aib-Leu-Ile-Cys]-NH₂ (GIP-319, SEQ ID NO: 278) of the following formula

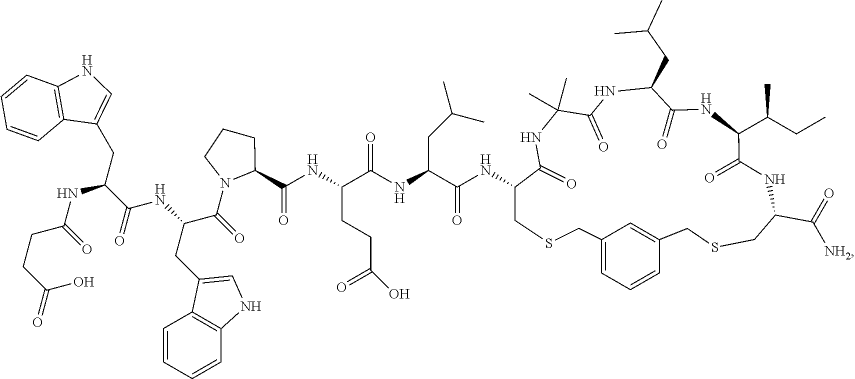 [SMILES: CC[C@H](C)[C@@H]1NC(=O)[C@H](CC(C)C)NC(=O)C(C)(C)NC(=O)[C@@H](NC(=O)[C@H](CC(C)C)NC(=O)[C@H](CCC(=O)O)NC(=O)[C@@H]2CCCN2C(=O)[C@H](Cc2c[nH]c3ccccc23)NC(=O)[C@H](Cc2c[nH]c3ccccc23)NC(=O)CCC(=O)O)CSCc2cccc(c2)CSC[C@@H](C(N)=O)NC1=O]

compound DOTA-Ttds-Asp-Trp-5Clw-Pro-Glu-leu-[Dap(3MeBz)-Aib-Leu-Ile-AET] (GIP-358, SEQ ID NO: 279) of the following formula

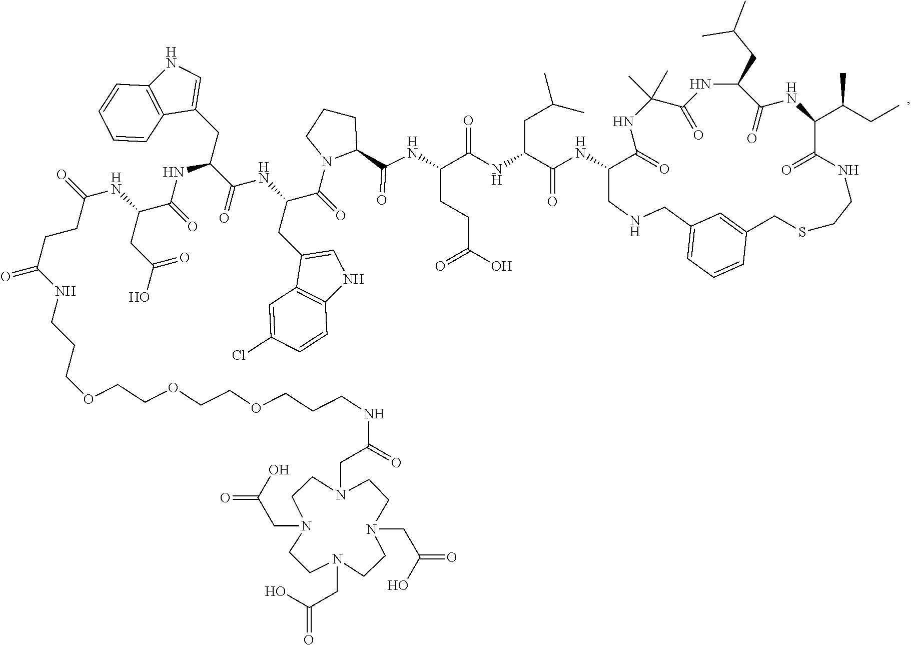 [SMILES: CC[C@H](C)[C@@H]1NC(=O)[C@H](CC(C)C)NC(=O)C(C)(C)NC(=O)[C@@H](NC(=O)[C@@H](CC(C)C)NC(=O)[C@H](CCC(=O)O)NC(=O)[C@@H]2CCCN2C(=O)[C@H](Cc2c[nH]c3ccc(Cl)cc23)NC(=O)[C@H](Cc2c[nH]c3ccccc23)NC(=O)[C@H](CC(=O)O)NC(=O)CCC(=O)NCCCOCCOCCOCCCNC(=O)CN2CCN(CC(=O)O)CCN(CC(=O)O)CCN(CC(=O)O)CC2)CNCc2cccc(c2)CSCCNC1=O]

compound DOTA-Ttds-Asp-Trp-5Clw-Pro-Glu-leu-[Cys(3CbBn)-Aib-Leu-Ile-en] (GIP-359, SEQ ID NO: 280) of the following formula

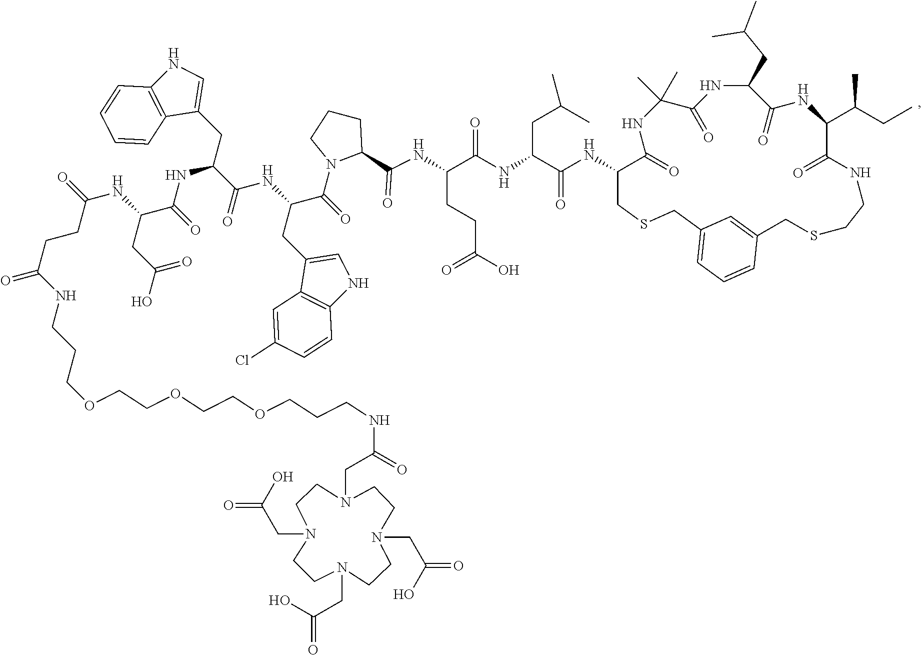 [SMILES: CC[C@H](C)[C@@H]1NC(=O)[C@H](CC(C)C)NC(=O)C(C)(C)NC(=O)[C@@H](NC(=O)[C@@H](CC(C)C)NC(=O)[C@H](CCC(=O)O)NC(=O)[C@@H]2CCCN2C(=O)[C@H](Cc2c[nH]c3ccc(Cl)cc23)NC(=O)[C@H](Cc2c[nH]c3ccccc23)NC(=O)[C@H](CC(=O)O)NC(=O)CCC(=O)NCCCOCCOCCOCCCNC(=O)CN2CCN(CC(=O)O)CCN(CC(=O)O)CCN(CC(=O)O)CC2)CSCc2cccc(c2)CSCCNC1=O]

compound Ac-Asp-Trp-5Clw-Hyp-Glu-leu-[Cys(mli)-Thp-Leu-Ile-Cys]-NH₂ (GIP-360, SEQ ID NO: 281) of the following formula

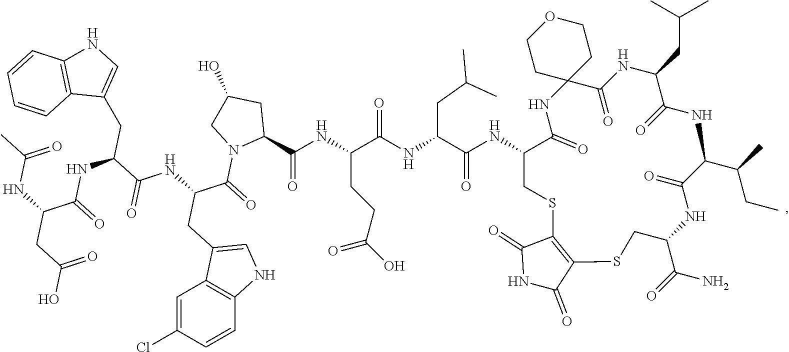 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CC(=O)O)NC(C)=O)CSC2=C(SC[C@@H](C(N)=O)NC1=O)C(=O)NC2=O]

compound Ac-Asp-Trp-5Clw-Hyp-Glu-leu-[Cys(mli)-Thp-Leu-Ile-Hcy]-NH₂ (GIP-361, SEQ ID NO: 282) of the following formula

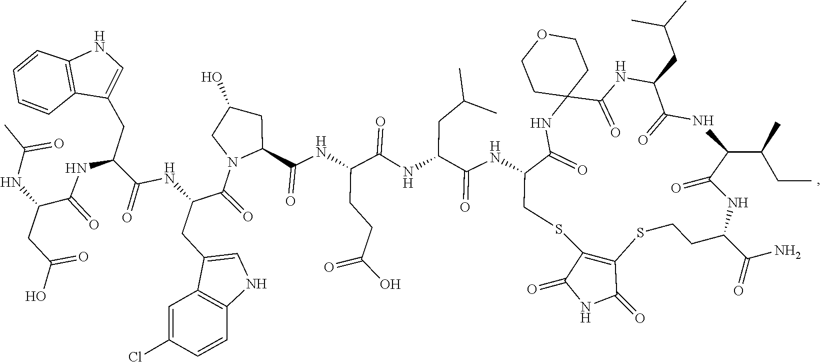 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CC(=O)O)NC(C)=O)CSC2=C(SCC[C@@H](C(N)=O)NC1=O)C(=O)NC2=O]

compound Ac-Asp-Trp-5Clw-Hyp-Glu-leu-[Hcy(mli)-Thp-Leu-Ile-Cys]-NH₂ (GIP-362, SEQ ID NO: 283) of the following formula

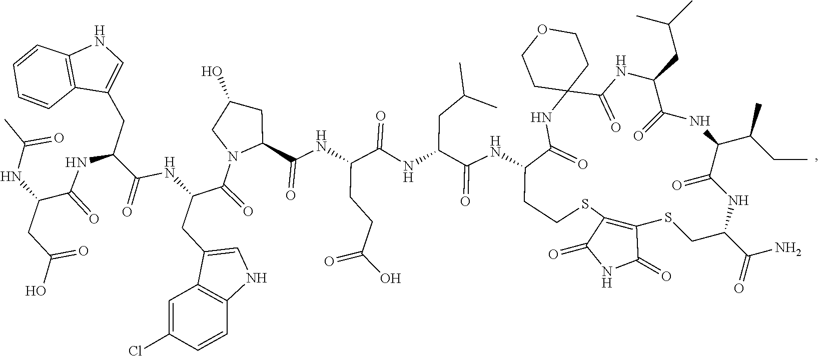 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CC(=O)O)NC(C)=O)CCSC2=C(SC[C@@H](C(N)=O)NC1=O)C(=O)NC2=O]

compound Ac-Asp-Trp-5Clw-Hyp-Glu-leu-[Hcy-Thp-Leu-Ile-Hcy]-NH₂ (GIP-363, SEQ ID NO: 284) of the following formula

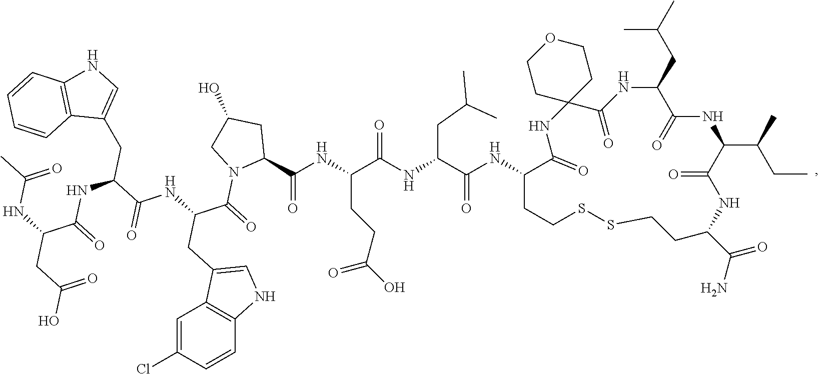 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CC(=O)O)NC(C)=O)CCSSCC[C@@H](C(N)=O)NC1=O]

compound AGLU-Glutar-Asp-Trp-5Clw-Tap-Glu-leu-[Cys(2Lut)-Apc(DOTAM)-Leu-Ile-Cys]-APAc-glu(AGLU)-NH₂ (GIP-370, SEQ ID NO: 285) of the following formula

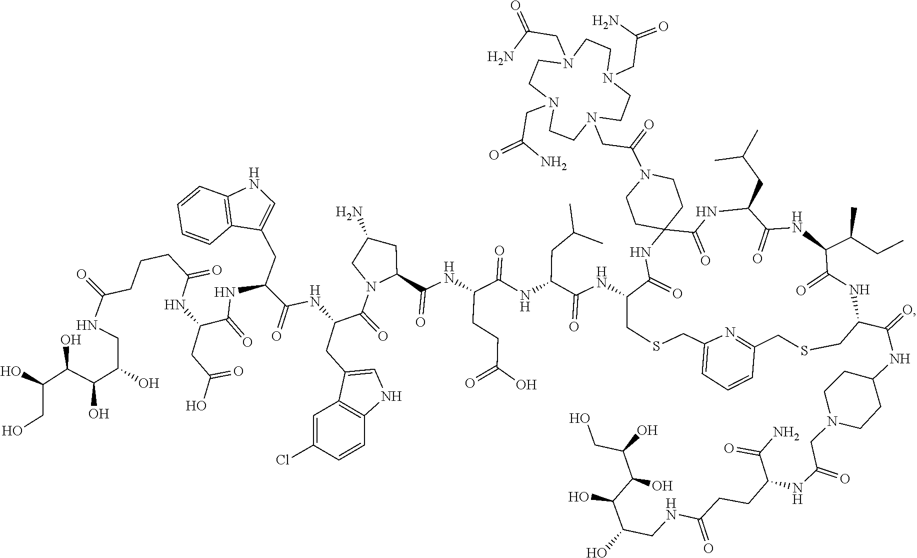 [SMILES: CC[C@H](C)[C@@H]1NC(=O)[C@H](CC(C)C)NC(=O)C2(CCN(C(=O)CN3CCN(CC(N)=O)CCN(CC(N)=O)CCN(CC(N)=O)CC3)CC2)NC(=O)[C@@H](NC(=O)[C@@H](CC(C)C)NC(=O)[C@H](CCC(=O)O)NC(=O)[C@@H]2C[C@@H](N)CN2C(=O)[C@H](Cc2c[nH]c3ccc(Cl)cc23)NC(=O)[C@H](Cc2c[nH]c3ccccc23)NC(=O)[C@H](CC(=O)O)NC(=O)CCCC(=O)NC[C@H](O)[C@@H](O)[C@H](O)[C@H](O)CO)CSCc2cccc(n2)CSC[C@@H](C(=O)NC2CCN(CC(=O)N[C@H](CCC(=O)NC[C@H](O)[C@@H](O)[C@H](O)[C@H](O)CO)C(N)=O)CC2)NC1=O]

compound AGLU-Glutar-Asp-Trp-5Clw-Tap-Glu-leu-[Cys(2Lut)-Amk(DOTA)-Leu-Ile-Cys]-APAc-glu(AGLU)-N_{H}(GIP-371, SEQ ID NO: 286) of the following formula

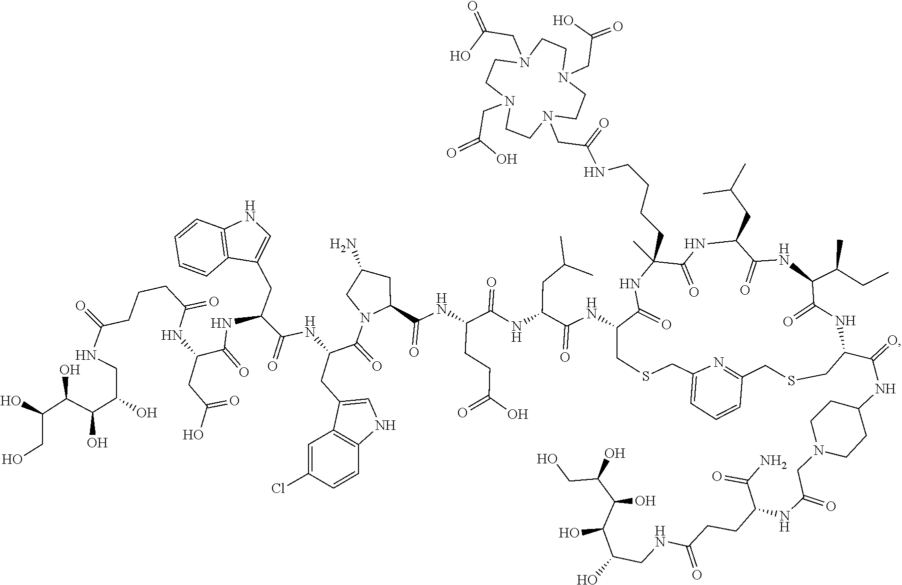 [SMILES: CC[C@H](C)[C@@H]1NC(=O)[C@H](CC(C)C)NC(=O)[C@](C)(CCCCNC(=O)CN2CCN(CC(=O)O)CCN(CC(=O)O)CCN(CC(=O)O)CC2)NC(=O)[C@@H](NC(=O)[C@@H](CC(C)C)NC(=O)[C@H](CCC(=O)O)NC(=O)[C@@H]2C[C@@H](N)CN2C(=O)[C@H](Cc2c[nH]c3ccc(Cl)cc23)NC(=O)[C@H](Cc2c[nH]c3ccccc23)NC(=O)[C@H](CC(=O)O)NC(=O)CCCC(=O)NC[C@H](O)[C@@H](O)[C@H](O)[C@H](O)CO)CSCc2cccc(n2)CSC[C@@H](C(=O)NC2CCN(CC(=O)N[C@H](CCC(=O)NC[C@H](O)[C@@H](O)[C@H](O)[C@H](O)CO)C(N)=O)CC2)NC1=O]

compound AGLU-Glutar-Asp-Trp-5Clw-Tap-Glu-leu-[Cys(2Lut)-Apc(DOTA)-Leu-Ile-Cys]-APAc-GLUSEROL (GIP-372, SEQ ID NO: 287) of the following formula

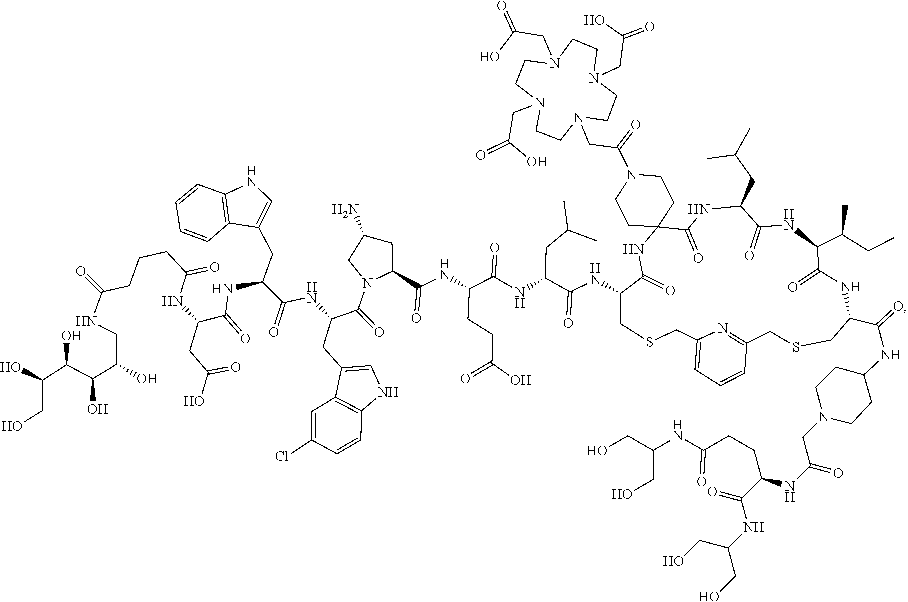 [SMILES: CC[C@H](C)[C@@H]1NC(=O)[C@H](CC(C)C)NC(=O)C2(CCN(C(=O)CN3CCN(CC(=O)O)CCN(CC(=O)O)CCN(CC(=O)O)CC3)CC2)NC(=O)[C@@H](NC(=O)[C@@H](CC(C)C)NC(=O)[C@H](CCC(=O)O)NC(=O)[C@@H]2C[C@@H](N)CN2C(=O)[C@H](Cc2c[nH]c3ccc(Cl)cc23)NC(=O)[C@H](Cc2c[nH]c3ccccc23)NC(=O)[C@H](CC(=O)O)NC(=O)CCCC(=O)NC[C@H](O)[C@@H](O)[C@H](O)[C@H](O)CO)CSCc2cccc(n2)CSC[C@@H](C(=O)NC2CCN(CC(=O)N[C@H](CCC(=O)NC(CO)CO)C(=O)NC(CO)CO)CC2)NC1=O]

compound NTASEROL-Asp-Trp-5Clw-Tap-Glu-leu-[Cys(2Lut)-Apc(DOTA)-Leu-Ile-Cys]-APAc-dap(NTASEROL)-NH₂ (GIP-373, SEQ ID NO: 288) of the following formula

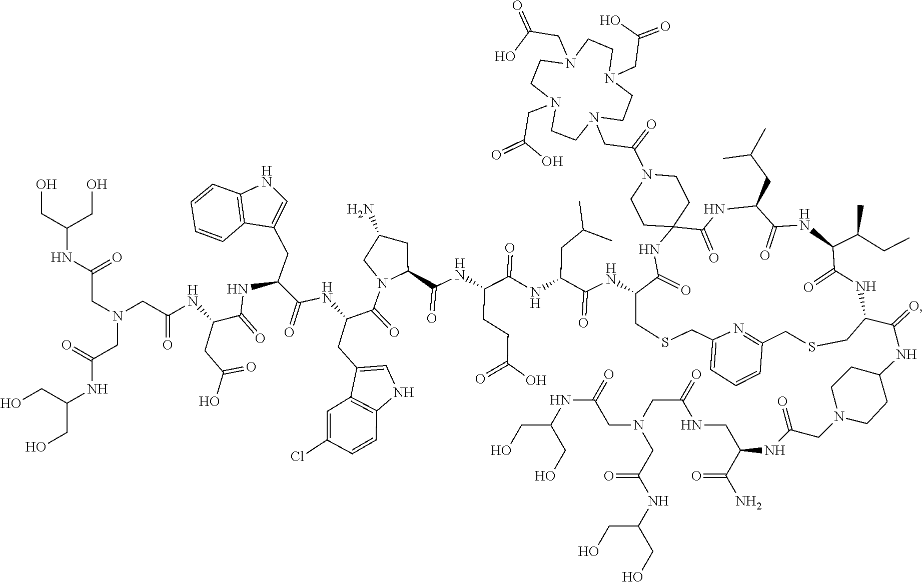 [SMILES: CC[C@H](C)[C@@H]1NC(=O)[C@H](CC(C)C)NC(=O)C2(CCN(C(=O)CN3CCN(CC(=O)O)CCN(CC(=O)O)CCN(CC(=O)O)CC3)CC2)NC(=O)[C@@H](NC(=O)[C@@H](CC(C)C)NC(=O)[C@H](CCC(=O)O)NC(=O)[C@@H]2C[C@@H](N)CN2C(=O)[C@H](Cc2c[nH]c3ccc(Cl)cc23)NC(=O)[C@H](Cc2c[nH]c3ccccc23)NC(=O)[C@H](CC(=O)O)NC(=O)CN(CC(=O)NC(CO)CO)CC(=O)NC(CO)CO)CSCc2cccc(n2)CSC[C@@H](C(=O)NC2CCN(CC(=O)N[C@H](CNC(=O)CN(CC(=O)NC(CO)CO)CC(=O)NC(CO)CO)C(N)=O)CC2)NC1=O]

compound NTASEROL-Asp-Trp-5Clw-Tap-Glu-leu-[Cys(2Lut)-Apc(DOTA)-Leu-Ile-Cys]-APAc-GLUSEROL (GIP-374, SEQ ID NO: 289) of the following formula

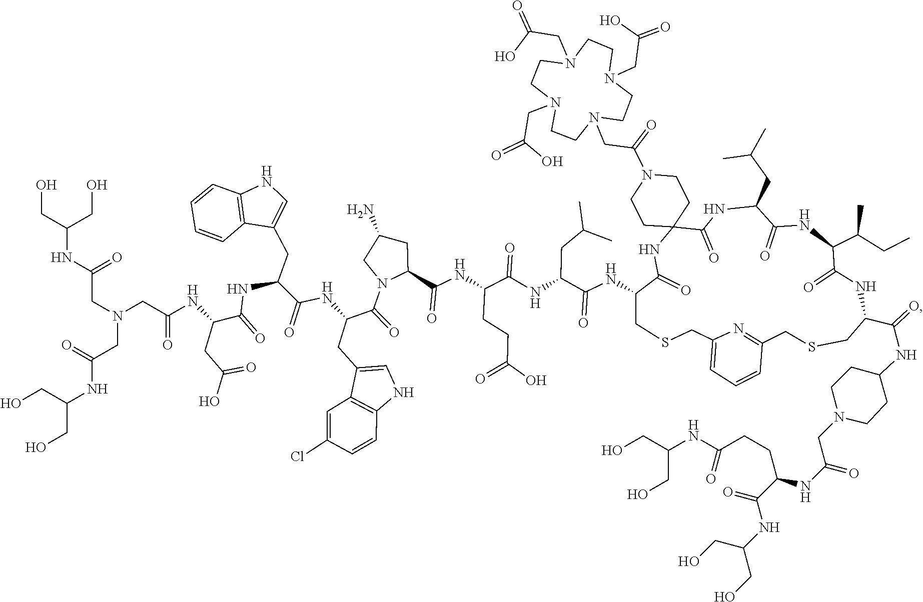 [SMILES: CC[C@H](C)[C@@H]1NC(=O)[C@H](CC(C)C)NC(=O)C2(CCN(C(=O)CN3CCN(CC(=O)O)CCN(CC(=O)O)CCN(CC(=O)O)CC3)CC2)NC(=O)[C@@H](NC(=O)[C@@H](CC(C)C)NC(=O)[C@H](CCC(=O)O)NC(=O)[C@@H]2C[C@@H](N)CN2C(=O)[C@H](Cc2c[nH]c3ccc(Cl)cc23)NC(=O)[C@H](Cc2c[nH]c3ccccc23)NC(=O)[C@H](CC(=O)O)NC(=O)CN(CC(=O)NC(CO)CO)CC(=O)NC(CO)CO)CSCc2cccc(n2)CSC[C@@H](C(=O)NC2CCN(CC(=O)N[C@H](CCC(=O)NC(CO)CO)C(=O)NC(CO)CO)CC2)NC1=O]

compound Ac-Asp-Trp-5Clw-Hyp-Lys-leu-[Cys(2Lut)-Thp-Leu-Ile-Cys]-NH₂ (GIP-375, SEQ ID NO: 290) of the following formula

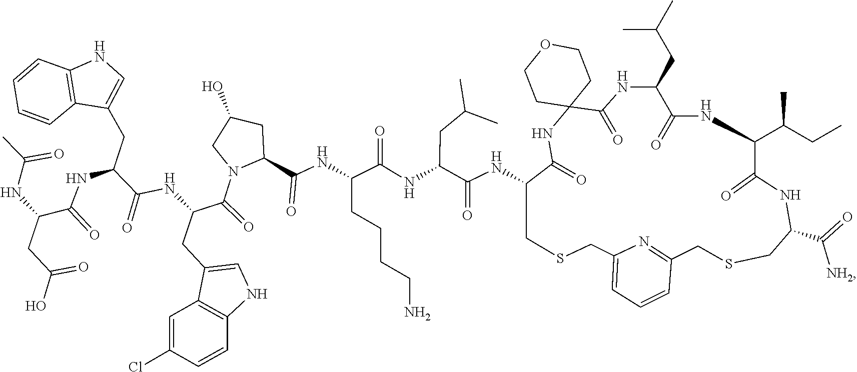 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCCCN)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CC(=O)O)NC(C)=O)CSCc2cccc(n2)CSC[C@@H](C(N)=O)NC1=O]

compound Ac-Asp-Trp-5Clw-Hyp-Arg-leu-[Cys(2Lut)-Thp-Leu-Ile-Cys]-NH₂ (GIP-376, SEQ ID NO: 291) of the following formula

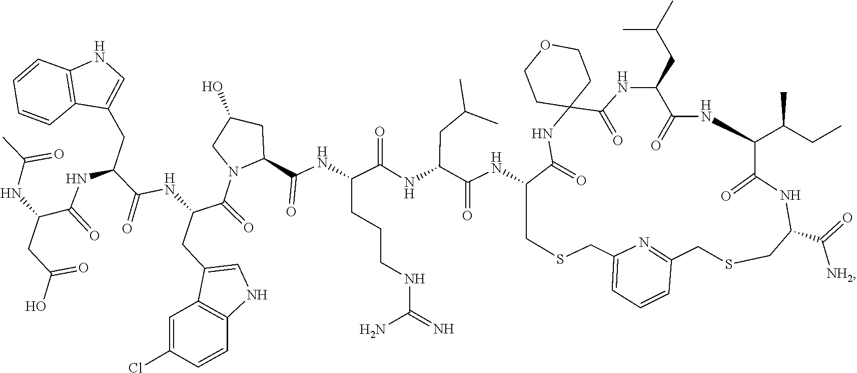 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCCNC(=N)N)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CC(=O)O)NC(C)=O)CSCc2cccc(n2)CSC[C@@H](C(N)=O)NC1=O]

compound Ac-Asp-Trp-5Clw-Hyp-His-leu-[Cys(2Lut)-Thp-Leu-Ile-Cys]-NH₂ (GIP-377, SEQ ID NO: 292) of the following formula

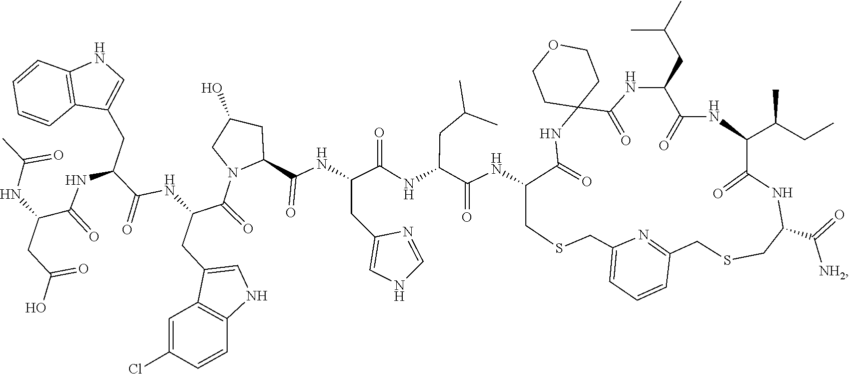 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](Cc2c[nH]cn2)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CC(=O)O)NC(C)=O)CSCc2cccc(n2)CSC[C@@H](C(N)=O)NC1=O]

compound Ac-Asp-Trp-5Clw-Hyp-Hly-leu-[Cys(2Lut)-Thp-Leu-Ile-Cys]-NH₂ (GIP-378, SEQ ID NO: 293) of the following formula

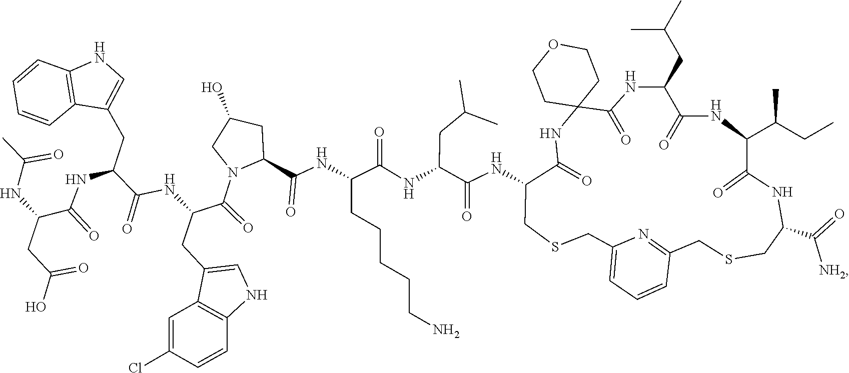 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCCCCN)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CC(=O)O)NC(C)=O)CSCc2cccc(n2)CSC[C@@H](C(N)=O)NC1=O]

compound Ac-Asp-5Clw-5Clw-Hyp-Glu-leu-[Cys(2Lut)-Thp-Leu-Ile-Cys]-NH₂ (GIP-379, SEQ ID NO: 294) of the following formula

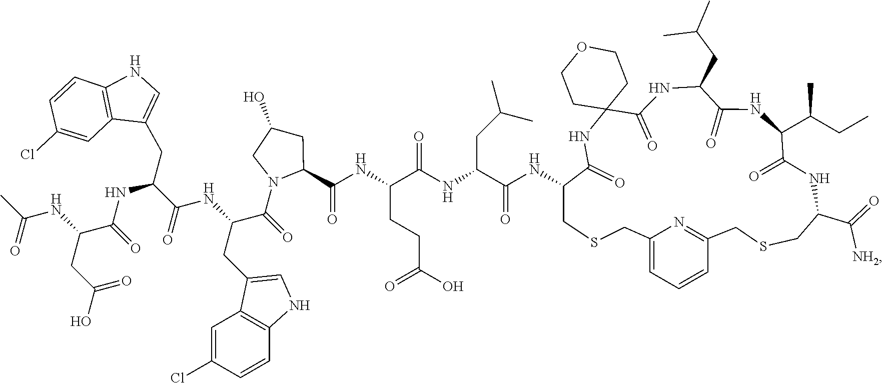 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3ccc(Cl)cc23)NC(=O)[C@H](CC(=O)O)NC(C)=O)CSCc2cccc(n2)CSC[C@@H](C(N)=O)NC1=O]

compound Ac-Asp-1Ni-5Clw-Hyp-Glu-leu-[Cys(2Lut)-Thp-Leu-Ile-Cys]-NH₂ (GIP-380, SEQ ID NO: 295) of the following formula

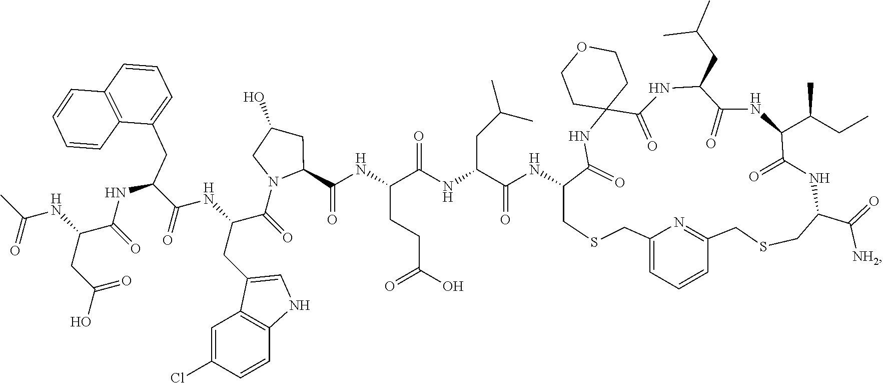 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2cccc3ccccc23)NC(=O)[C@H](CC(=O)O)NC(C)=O)CSCc2cccc(n2)CSC[C@@H](C(N)=O)NC1=O]

compound Ac-asp-Trp-5Clw-Hyp-Glu-leu-[Cys(2Lut)-Thp-Leu-Ile-Cys]-NH₂ (GIP-381, SEQ ID NO: 296) of the following formula

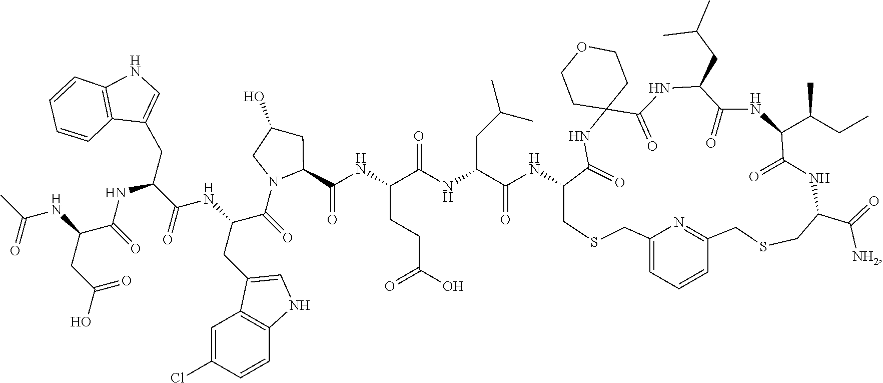 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@@H](CC(=O)O)NC(C)=O)CSCc2cccc(n2)CSC[C@@H](C(N)=O)NC1=O]

compound Ac-Asp-5Fw-5Clw-Hyp-Glu-leu-[Cys(2Lut)-Thp-Leu-Ile-Cys]-NH₂ (GIP-382, SEQ ID NO: 297) of the following formula

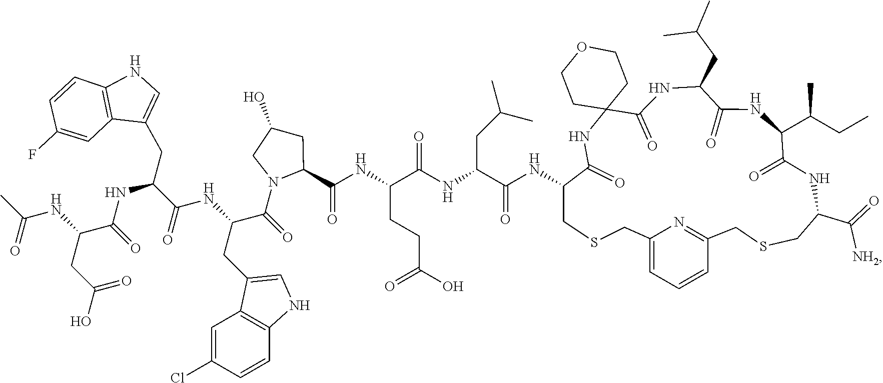 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3ccc(F)cc23)NC(=O)[C@H](CC(=O)O)NC(C)=O)CSCc2cccc(n2)CSC[C@@H](C(N)=O)NC1=O]

compound Ac-Asp-Trp-5Clw-Hyp-Glu-leu-[Cys(2Lut)-Thp-Cha-Ile-Cys]-NH₂ (GIP-383, SEQ ID NO: 298) of the following formula

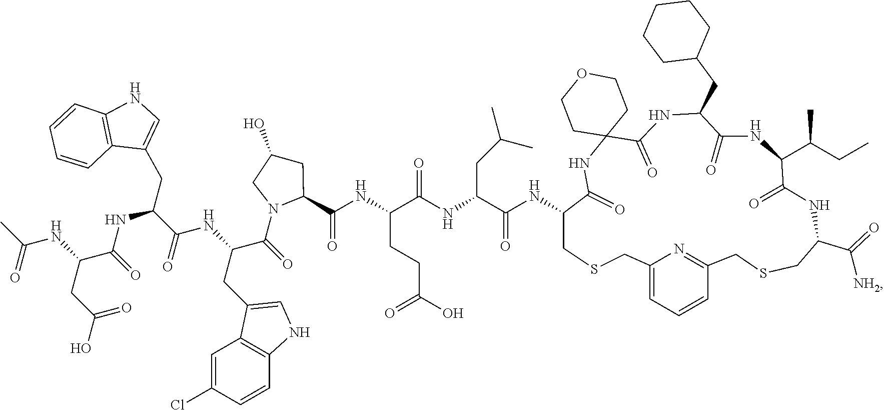 [SMILES: CC[C@H](C)[C@@H]1NC(=O)[C@H](CC2CCCCC2)NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CC(=O)O)NC(C)=O)CSCc2cccc(n2)CSC[C@@H](C(N)=O)NC1=O]

compound Ac-Asp-Trp-5Clw-Hyp-Ser-leu-[Cys(2Lut)-Thp-Leu-Ile-Cys]-NH₂ (GIP-384, SEQ ID NO: 299) of the following formula

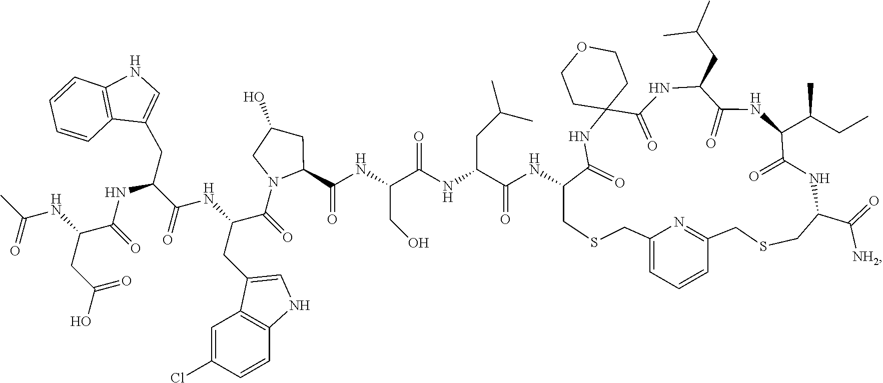 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CO)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CC(=O)O)NC(C)=O)CSCc2cccc(n2)CSC[C@@H](C(N)=O)NC1=O]

compound Ac-Asp-Trp-5Clw-Hyp-Val-leu-[Cys(2Lut)-Thp-Leu-Ile-Cys]-NH₂ (GIP-385, SEQ ID NO: 300) of the following formula

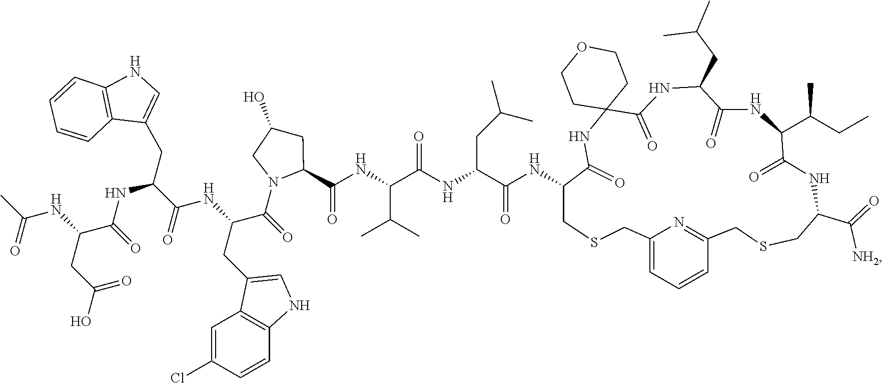 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@@H](NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CC(=O)O)NC(C)=O)C(C)C)CSCc2cccc(n2)CSC[C@@H](C(N)=O)NC1=O]

compound Ac-Asp-Trp-5Clw-Hyp-Ala-leu-[Cys(2Lut)-Thp-Leu-Ile-Cys]-NH₂ (GIP-386, SEQ ID NO: 301) of the following formula

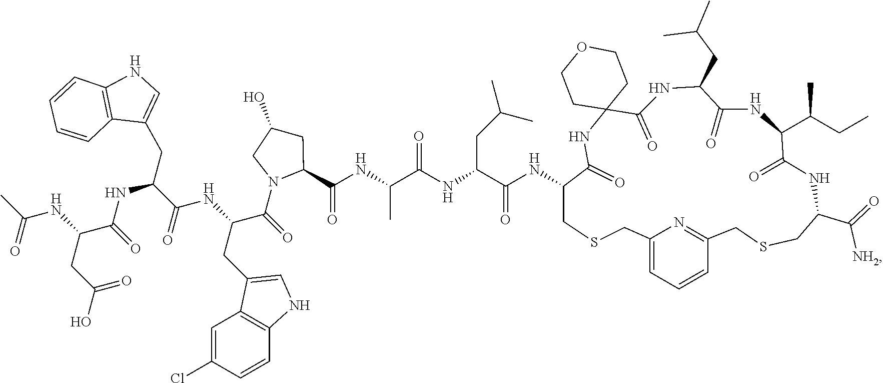 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](C)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CC(=O)O)NC(C)=O)CSCc2cccc(n2)CSC[C@@H](C(N)=O)NC1=O]

compound DOTA-Ttds-Asp-Ocf-5Clw-Hyp-Glu-leu-[Cys(2Lut)-Thp-Leu-Ile-Cys]-NH₂ (GIP-387, SEQ ID NO: 302) of the following formula

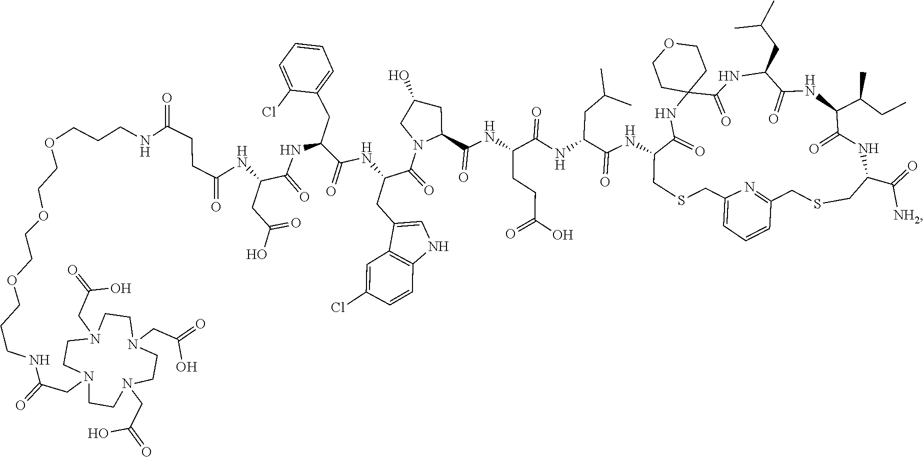 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2ccccc2Cl)NC(=O)[C@H](CC(=O)O)NC(=O)CCC(=O)NCCCOCCOCCOCCCNC(=O)CN2CCN(CC(=O)O)CCN(CC(=O)O)CCN(CC(=O)O)CC2)CSCc2cccc(n2)CSC[C@@H](C(N)=O)NC1=O]

compound DOTA-Ttds-Asp-Trp-5Clw-Hyp-Glu-leu-[Cys(2Lut)-Thp-Leu-Phe-Cys]-NH₂ (GIP-388, SEQ ID NO: 303) of the following formula

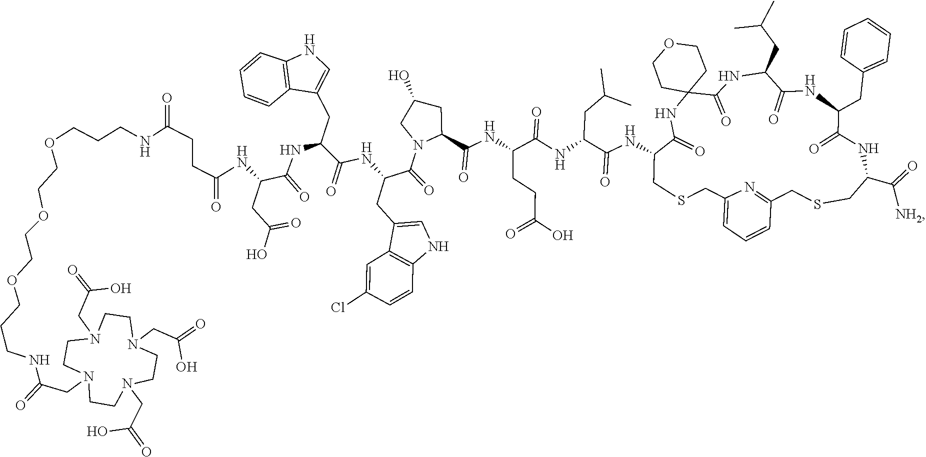 [SMILES: CC(C)C[C@@H]1NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CC(=O)O)NC(=O)CCC(=O)NCCCOCCOCCOCCCNC(=O)CN2CCN(CC(=O)O)CCN(CC(=O)O)CCN(CC(=O)O)CC2)CSCc2cccc(n2)CSC[C@@H](C(N)=O)NC(=O)[C@H](Cc2ccccc2)NC1=O]

compound Ac-Asp-Trp-5Clw-Hyp-Glu-leu-[Cys(2Lut)-Thp-Leu-Phe-Cys]-lys (DOTA)-NH₂ (GIP-389, SEQ ID NO: 304) of the following formula

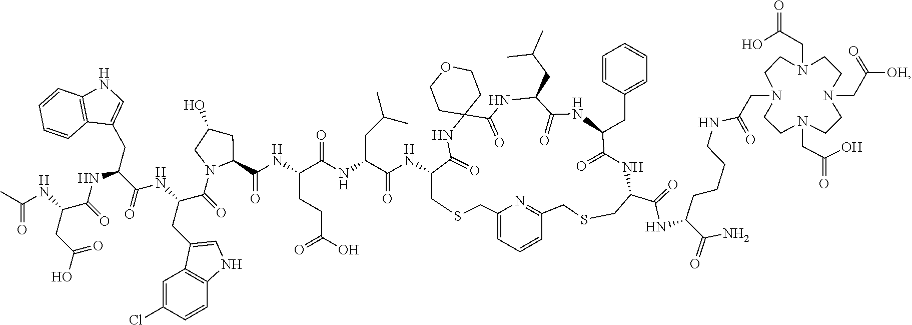 [SMILES: CC(=O)N[C@@H](CC(=O)O)C(=O)N[C@@H](Cc1c[nH]c2ccccc12)C(=O)N[C@@H](Cc1c[nH]c2ccc(Cl)cc12)C(=O)N1C[C@H](O)C[C@H]1C(=O)N[C@@H](CCC(=O)O)C(=O)N[C@H](CC(C)C)C(=O)N[C@H]1CSCc2cccc(n2)CSC[C@@H](C(=O)N[C@H](CCCCNC(=O)CN2CCN(CC(=O)O)CCN(CC(=O)O)CCN(CC(=O)O)CC2)C(N)=O)NC(=O)[C@H](Cc2ccccc2)NC(=O)[C@H](CC(C)C)NC(=O)C2(CCOCC2)NC1=O]

compound Ac-Asp-Ocf-5Clw-Tap (DOTA)-Glu-leu-[Cys(2Lut)-Thp-Leu-Ile-Cys]-NH₂ (GIP-390, SEQ ID NO: 305) of the following formula

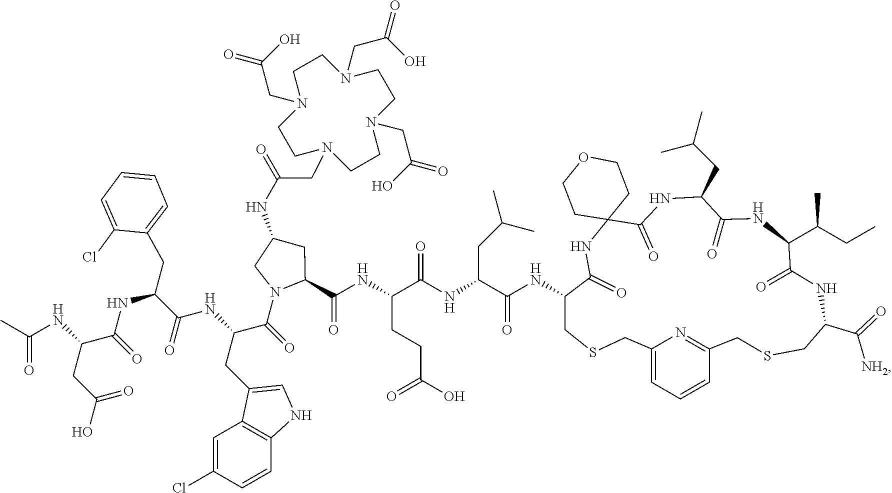 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](NC(=O)CN3CCN(CC(=O)O)CCN(CC(=O)O)CCN(CC(=O)O)CC3)CN2C(=O)[C@H](Cc2c[nH]c3ccc(Cl)cc23)NC(=O)[C@H](Cc2ccccc2Cl)NC(=O)[C@H](CC(=O)O)NC(C)=O)CSCc2cccc(n2)CSC[C@@H](C(N)=O)NC1=O]

compound Ac-Asp-Trp-5Clw-Tap (DOTA)-Glu-leu-[Cys(2Lut)-Thp-Leu-Phe-Cys]-NH₂ (GIP-391, SEQ ID NO: 306) of the following formula

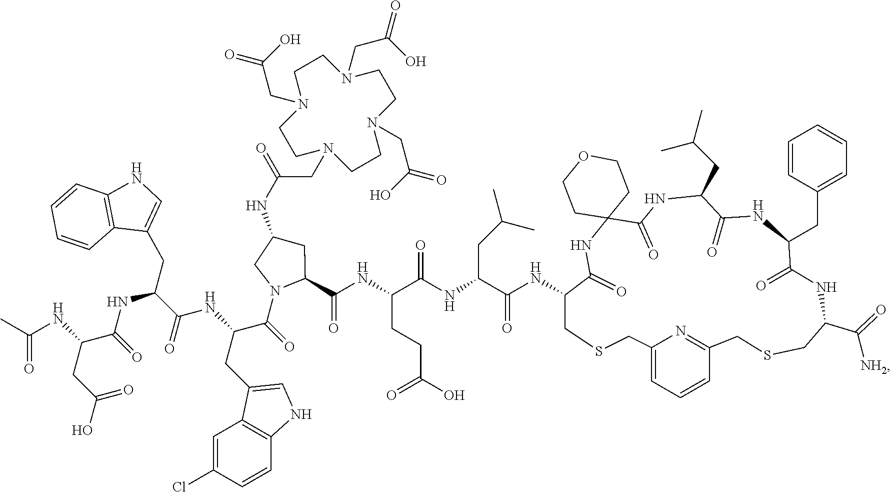 [SMILES: CC(=O)N[C@@H](CC(=O)O)C(=O)N[C@@H](Cc1c[nH]c2ccccc12)C(=O)N[C@@H](Cc1c[nH]c2ccc(Cl)cc12)C(=O)N1C[C@H](NC(=O)CN2CCN(CC(=O)O)CCN(CC(=O)O)CCN(CC(=O)O)CC2)C[C@H]1C(=O)N[C@@H](CCC(=O)O)C(=O)N[C@H](CC(C)C)C(=O)N[C@H]1CSCc2cccc(n2)CSC[C@@H](C(N)=O)NC(=O)[C@H](Cc2ccccc2)NC(=O)[C@H](CC(C)C)NC(=O)C2(CCOCC2)NC1=O]

compound Ac-Asp-Ocf-5Clw-Hyp-Glu-leu-[Cys(2Lut)-Apc(DOTA)-Leu-Ile-Cys]-NH₂ (GIP-392, SEQ ID NO: 307) of the following formula

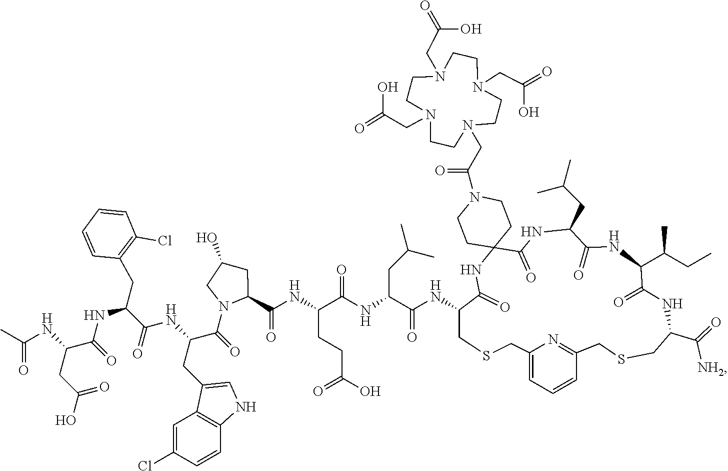 [SMILES: CC[C@H](C)[C@@H]1NC(=O)[C@H](CC(C)C)NC(=O)C2(CCN(C(=O)CN3CCN(CC(=O)O)CCN(CC(=O)O)CCN(CC(=O)O)CC3)CC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2ccccc2Cl)NC(=O)[C@H](CC(=O)O)NC(C)=O)CSCc2cccc(n2)CSC[C@@H](C(N)=O)NC1=O]

compound Ac-Asp-Trp-5Clw-Hyp-Glu-leu-[Cys(2Lut)-Apc(DOTA)-Leu-Phe-Cys]-NH₂ (GIP-393, SEQ ID NO: 308) of the following formula

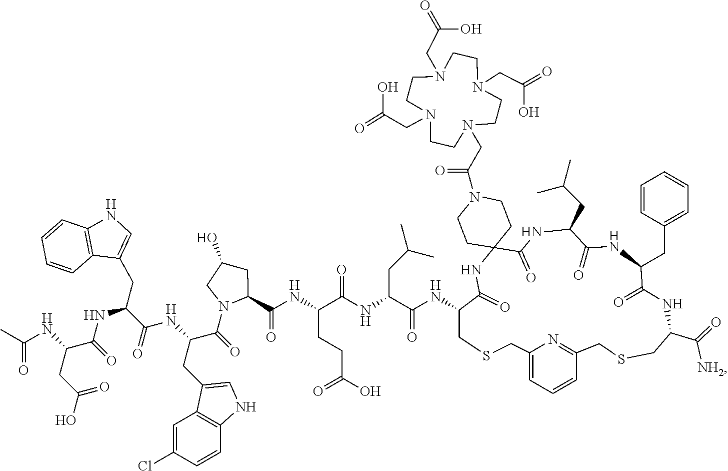 [SMILES: CC(=O)N[C@@H](CC(=O)O)C(=O)N[C@@H](Cc1c[nH]c2ccccc12)C(=O)N[C@@H](Cc1c[nH]c2ccc(Cl)cc12)C(=O)N1C[C@H](O)C[C@H]1C(=O)N[C@@H](CCC(=O)O)C(=O)N[C@H](CC(C)C)C(=O)N[C@H]1CSCc2cccc(n2)CSC[C@@H](C(N)=O)NC(=O)[C@H](Cc2ccccc2)NC(=O)[C@H](CC(C)C)NC(=O)C2(CCN(C(=O)CN3CCN(CC(=O)O)CCN(CC(=O)O)CCN(CC(=O)O)CC3)CC2)NC1=O]

compound Ac-Asp-Trp-1Ni-Tap(DOTA)-Glu-Hle-[Cys(3MeBn)-Aib-Leu-Ile-Cys]-NH₂ (GIP-394, SEQ ID NO: 309) of the following formula

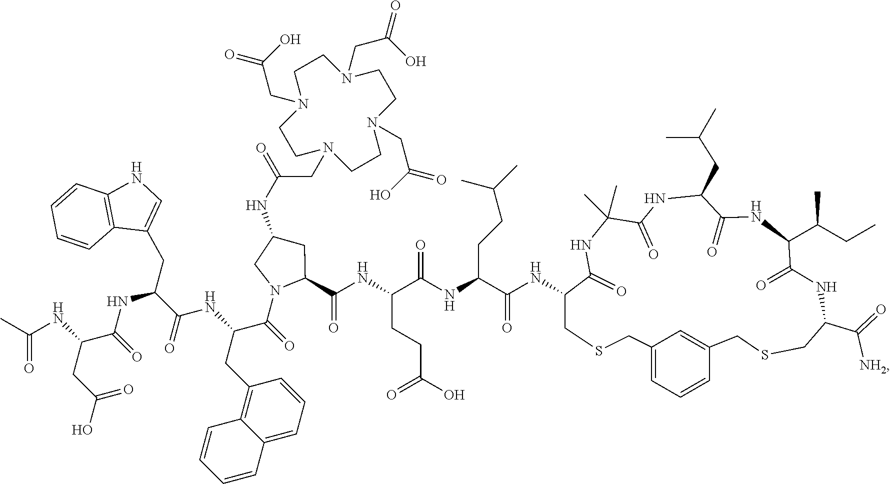 [SMILES: CC[C@H](C)[C@@H]1NC(=O)[C@H](CC(C)C)NC(=O)C(C)(C)NC(=O)[C@@H](NC(=O)[C@H](CCC(C)C)NC(=O)[C@H](CCC(=O)O)NC(=O)[C@@H]2C[C@@H](NC(=O)CN3CCN(CC(=O)O)CCN(CC(=O)O)CCN(CC(=O)O)CC3)CN2C(=O)[C@H](Cc2cccc3ccccc23)NC(=O)[C@H](Cc2c[nH]c3ccccc23)NC(=O)[C@H](CC(=O)O)NC(C)=O)CSCc2cccc(c2)CSC[C@@H](C(N)=O)NC1=O]

compound Ac-Asp-Trp-5Clw-Hyp-Har-leu-[Cys(2Lut)-Thp-Leu-Ile-Cys]-NH₂ (GIP-395, SEQ ID NO: 310) of the following formula

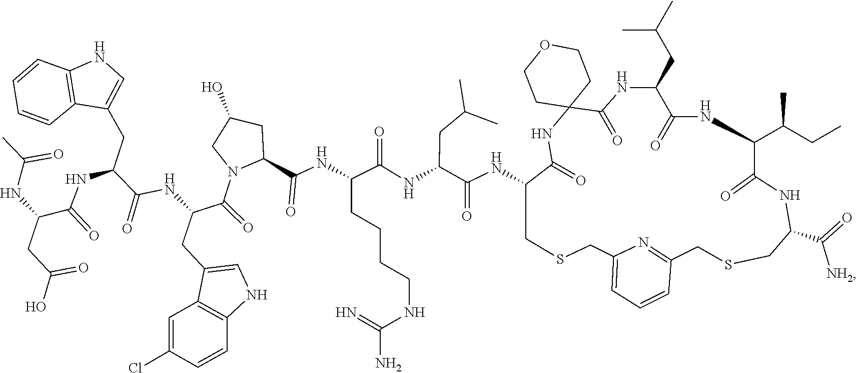 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCCCNC(=N)N)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CC(=O)O)NC(C)=O)CSCc2cccc(n2)CSC[C@@H](C(N)=O)NC1=O]

compound Ac-Asp-Trp-5Clw-Hyp-Phe-leu-[Cys(2Lut)-Thp-Leu-Ile-Cys]-NH₂ (GIP-396, SEQ ID NO: 311) of the following formula

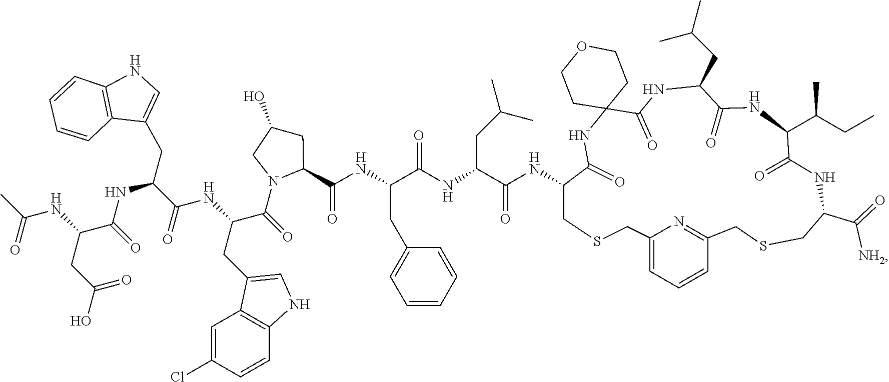 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](Cc2ccccc2)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CC(=O)O)NC(C)=O)CSCc2cccc(n2)CSC[C@@H](C(N)=O)NC1=O]

compound Ac-Asp-Trp-5Clw-Hyp-Trp-leu-[Cys(2Lut)-Thp-Leu-Ile-Cys]-NH₂ (GIP-397, SEQ ID NO: 312) of the following formula

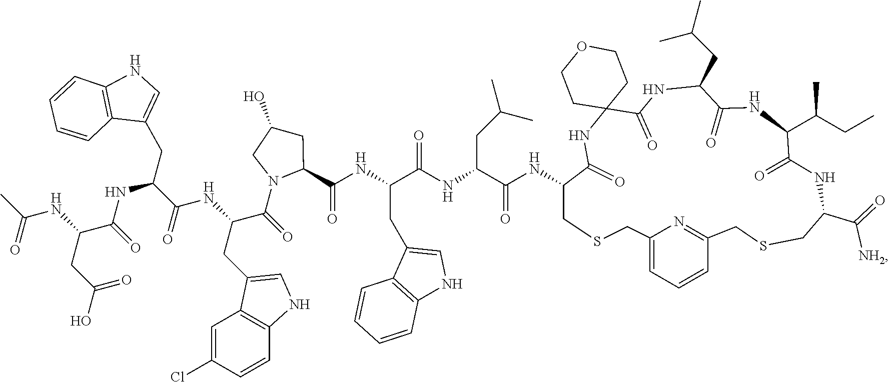 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](Cc2c[nH]c3ccccc23)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CC(=O)O)NC(C)=O)CSCc2cccc(n2)CSC[C@@H](C(N)=O)NC1=O]

compound Ac-Asp-Trp-5Clw-Hyp-Pro-leu-[Cys(2Lut)-Thp-Leu-Ile-Cys]-NH₂ (GIP-398, SEQ ID NO: 313) of the following formula

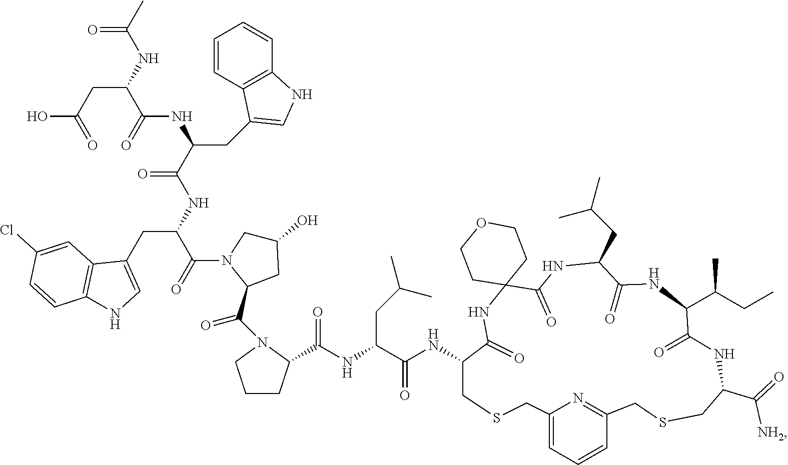 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@@H]2CCCN2C(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CC(=O)O)NC(C)=O)CSCc2cccc(n2)CSC[C@@H](C(N)=O)NC1=O]

compound Ac-Asp-Ocf-5Clw-Hyp-Glu-leu-[Cys(2Lut)-Thp-Leu-Ile-Cys]-lys(DOT A)-NH₂ (GIP-399, SEQ ID NO: 314) of the following formula

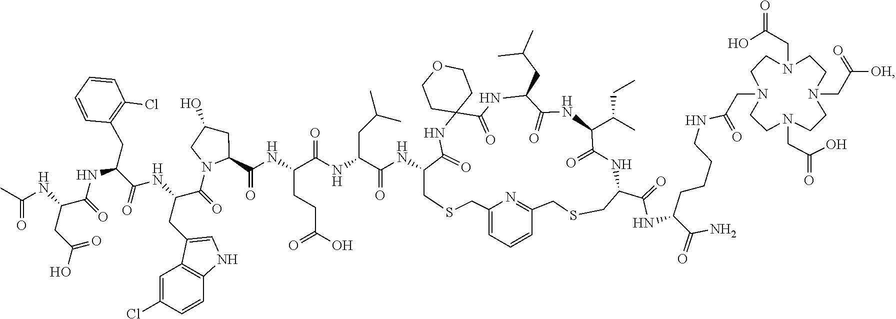 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2ccccc2Cl)NC(=O)[C@H](CC(=O)O)NC(C)=O)CSCc2cccc(n2)CSC[C@@H](C(=O)N[C@H](CCCCNC(=O)CN2CCN(CC(=O)O)CCN(CC(=O)O)CCN(CC(=O)O)CC2)C(N)=O)NC1=O]

and compound Ac-Asp-Trp-1Ni-Pro-Glu-Hle-[Cys(3MeBn)-Apc(DOTA)-Leu-Ile-Cys]-NH₂ (GIP-400, SEQ ID NO: 315) of the following formula

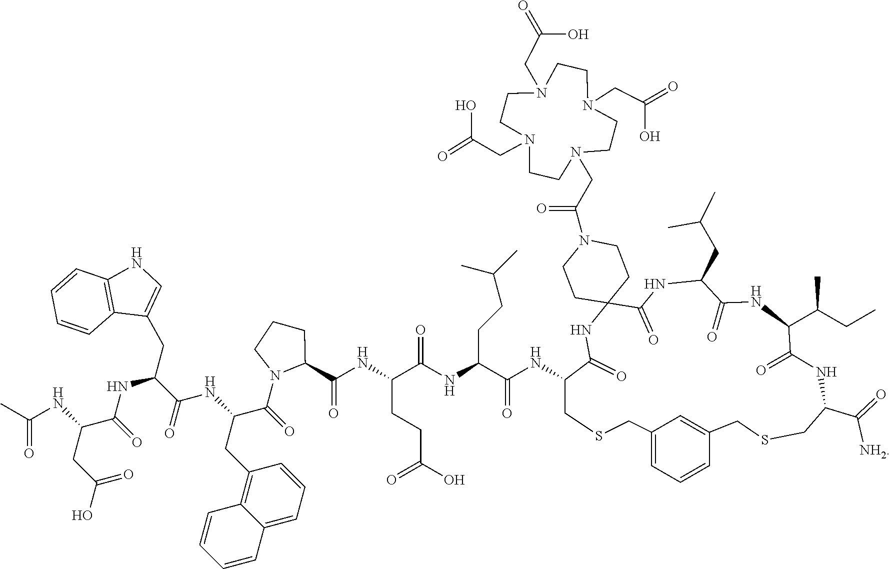 [SMILES: CC[C@H](C)[C@@H]1NC(=O)[C@H](CC(C)C)NC(=O)C2(CCN(C(=O)CN3CCN(CC(=O)O)CCN(CC(=O)O)CCN(CC(=O)O)CC3)CC2)NC(=O)[C@@H](NC(=O)[C@H](CCC(C)C)NC(=O)[C@H](CCC(=O)O)NC(=O)[C@@H]2CCCN2C(=O)[C@H](Cc2cccc3ccccc23)NC(=O)[C@H](Cc2c[nH]c3ccccc23)NC(=O)[C@H](CC(=O)O)NC(C)=O)CSCc2cccc(c2)CSC[C@@H](C(N)=O)NC1=O]

Embodiment 196. The compound of any one of embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 138, 139, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194 and 195, preferably the compound of embodiment 195, wherein the compound
a) is selected from the group consisting of GIP-001 to GIP-031, GIP-033 to GIP-056, GIP-059 to GIP-224, GIP-226 to GIP-263, GIP-265 to GIP-267, GIP-270, GIP-271, GIP-273, GIP-274, GIP-276, GIP-277, GIP-279, GIP-281, GIP-283, GIP-315, GIP-319, GIP-358, GIP-359, GIP-363 and GIP-370 to GIP-400,
b) preferably is selected from the group consisting of GIP-001 to GIP-031, GIP-033 to GIP-056, GIP-059 to GIP-224, GIP-226 to GIP-263, GIP-265 to GIP-267 and GIP-370 to GIP-386,
c) more preferably is selected from the group consisting of GIP-001 to GIP-031, GIP-033 to GIP-056, GIP-059 to GIP-071 and GIP-370 to GIP-374,
and
d) most preferably is selected from the group consisting of GIP-001 to GIP-024 and GIP-370 to GIP-374.

Embodiment 197. The compound of any one of embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 138, 139, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195 and 196, preferably the compound of any one of embodiments 195 and 196, wherein the compound
a) is selected from the group consisting of GIP-001 to GIP-031, GIP-033 to GIP-056, GIP-072 to GIP-145, GIP-147, GIP-270, GIP-271, GIP-273, GIP-274, GIP-276, GIP-277, GIP-279, GIP-281, GIP-283, GIP-358, GIP-359, GIP-370 to GIP-374, GIP-387 to GIP GIP-394, GIP-399 and GIP-400,
b) preferably is selected from the group consisting of GIP-001 to GIP-031, GIP-033 to GIP-056, GIP-072 to GIP-145, GIP-147 and GIP-370 to GIP-374,
c) more preferably is selected from the group consisting of GIP-001 to GIP-031, GIP-033 to GIP-056 and GIP-370 to GIP-374,
and
d) most preferably is selected from the group consisting of GIP-001 to GIP-024 and GIP-370 to GIP-374.

Embodiment 198. The compound of any one of embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 138, 139, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196 and 197, preferably the compound of any one of embodiments 195, 196 and 197, wherein the compound
a) is selected from the group consisting of GIP-001 to GIP-031, GIP-033 to GIP-056, GIP-072 to GIP-145, GIP-147 and GIP-370 to GIP-374,
b) preferably is selected from the group consisting of GIP-001 to GIP-031, GIP-033 to GIP-056 and GIP-370 to GIP-374,
c) more preferably is selected from the group consisting of GIP-001 to GIP-024 and GIP-370 to GIP-374.
and
c) most preferably is selected from the group consisting of GIP-001 to GIP-007.

Embodiment 199. The compound of any one of embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 138, 139, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197 and 198, wherein any S-atom, which can be oxidized, preferably any sulfur atom of a thioether group, is present as —S—, —S(O)— or —S(O₂)— or mixture thereof.

Embodiment 200. The compound of any one of embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 138, 139, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198 and 199, wherein the compound comprises a diagnostically active radio nuclide, wherein the radionuclide
a) is a radionuclide used for diagnosis,
b) preferably is selected from the group, but not limited to, comprising ⁴³Sc, ⁴⁴Sc, ⁵¹Mn, ⁵²Mn, ⁶⁴Cu, ⁶⁷Ga, ⁶⁸Ga, ⁸⁶Y, ⁸⁹Zr, ^{94m}Tc, ^{99m}Tc, ¹¹¹In, ¹⁵²Tb, ¹⁵⁵Tb, ¹⁷⁷Lu, ²⁰¹Tl, ²⁰³Pb, ¹⁸F, ⁷⁶Br, ⁷⁷Br, ¹²³I, ¹²⁴I, and ¹²⁵I,
c) more preferably is selected from the group comprising ⁴³Sc, ⁴⁴Sc, ⁶⁴Cu, ⁶⁷Ga, ⁶⁸Ga, ⁸⁶Y, ⁸⁹Zr, ¹¹¹In, ¹⁵²Tb, ¹⁵⁵Tb, and ²⁰³Pb,
and
d) most preferably is selected from the group comprising ⁶⁴Cu, ⁶⁸Ga, ¹¹¹In and ²⁰³Pb.

Embodiment 201. The compound of any one of embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 138, 139, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198 and 199, wherein the compound comprises a therapeutically active radionuclide, wherein the radionuclide
a) is a radionuclide used for therapy,
b) preferably is selected from the group comprising ⁴⁷Sc, ⁶⁷Cu, ⁸⁹Sr, ⁹⁰Y, ¹¹¹In, ¹⁵³Sm, ¹⁴⁹Tb, ¹⁶¹Tb, ¹⁷⁷Lu, ¹⁸⁶Re, ¹⁸⁸Re, ²¹²Pb, ²¹³Bi, ²²³Ra, ²²⁵Ac, ²²⁶Th, ²²⁷Th, ¹³¹I, and ²¹¹At,
c) more preferably is selected from the group comprising ⁴⁷Sc, ⁶⁷Cu, ⁹⁰Y, ¹⁷⁷Lu, ²¹²Pb, ²¹³Bi, ²²⁵Ac, and ²²⁷Th,
and
d) most preferably is selected from the group comprising ⁹⁰Y, ¹⁷⁷Lu, ²¹²Pb, ²²⁵Ac, and ²²⁷Th.

Embodiment 202. The compound of any one of embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 138, 139, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200 and 201, for use in a method of diagnosing a disease.

Embodiment 203. The compound of any one of embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 138, 139, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201 and 202, for use in a method for the treatment of a disease.

Embodiment 204. The compound of any one of embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 138, 139, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200 and 201, for use in a method for the identification of a subject, wherein the subject is likely to respond or likely not to respond to a treatment of a disease, wherein the method for the identification of a subject comprises carrying out a method of diagnosing a disease using a compound of any one of embodiments 1 to 201.

Embodiment 205. The compound of any one of embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 138, 139, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200 and 201, for use in a method for the selection of a subject from a group of subjects, wherein the subject is likely to respond or likely not to respond to a treatment of a disease, wherein the method for the selection of a subject from a group of subjects comprises carrying out a method of diagnosing a disease using a compound of any one of embodiments 1 to 201.

Embodiment 206. The compound of any one of embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 138, 139, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200 and 201, for use in a method for the stratification of a group of subjects into subjects which are likely to respond to a treatment of a disease, and into subjects which are not likely to respond to a treatment of a disease, wherein the method for the stratification of a group of subjects comprises carrying out a method of diagnosing a disease using a compound of any one of embodiments 1 to 201.

Embodiment 207. The compound for use of any one of embodiments 201, 202, 203, 204 and 205, wherein the disease is cancer.

Embodiment 208. The compound for use of embodiment 207, wherein the cancer is expressing Gastric Inhibitory Peptide Receptor (GIPR), preferably, the cancer is overexpressing GIPR.

Embodiment 209. The compound for use of any one of embodiments 207 and 208, wherein the cancer is a neuroendocrine cancer or a neuroendocrine neoplasm.

Embodiment 210. The compound for use of any one of embodiments 207, 208 209, wherein the cancer is selected from the group consisting of medullary thyroid cancer, gastrointestinal neuroendocrine neoplasms, ileal neuroendocrine neoplasms, pancreatic neuroendocrine neoplasms, nonfunctioning neuroendocrine neoplasms, Insulinomas, Gastrinomas, Glucagonomas, VIPomas, Somatostatinoma, ACTHoma, lung neuroendocrine neoplasms, typical carcinoid tumors, atypical carcinoid tumors, small cell carcinoma of the lung, large cell carcinoma of the lung, thymic neuroendocrine tumors, Merkel cell carcinoma, Pheochromocytoma of the adrenal gland, adrenal cancer, parathyroid cancer, paraganglioma, pituitary gland tumors, neuroendocrine tumors of the ovaries and neuroendocrine tumors of the testicles.

Embodiment 211. The compound for use of any one of embodiments 203, 204 and 205, wherein the disease is a metabolic disease.

Embodiment 212. The compound for use of embodiment 211, wherein a diseased cell and/or a diseased tissue, preferably a diseased cell and/or a diseased tissue of the metabolic disease expresses GIPR.

Embodiment 213. The compound for use of embodiment 212, wherein the diseased cell and/or the diseased tissue overexpresses GIPR.

Embodiment 214. The compound for use of any one of embodiments 212 and 213, wherein the diseased cell is a pancreatic cell.

Embodiment 215. The compound for use of any one of embodiments 211, 212, 213 and 214, wherein the metabolic disease is selected from the group consisting of type 2 diabetes, type 1 diabetes, metabolic syndrome, insulin resistance, dyslipidemia, impaired fasting glucose, and impaired glucose tolerance.

Embodiment 216. A composition comprising a compound of any one of embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 138, 139, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200 and 201 and a pharmaceutically acceptable excipient.

Embodiment 217. The composition of embodiment 216, wherein the composition is a pharmaceutical composition.

Embodiment 218. A kit comprising a compound of any one of embodiments 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 138, 139, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200 and 201 and one or more optional excipient(s) and optionally one or more device(s).

Embodiment 219. The kit of embodiment 218, wherein the device(s) is/are selected from the group comprising a labeling device, a purification device, a handling device, a radioprotection device, an analytical device or an administration device.

In accordance with the present invention, the compound of the present invention as subject to embodiment 1 will also be referred to herein as the first aspect of the present invention.

In accordance with the present invention, the compound of the present invention according to the first aspect of the present invention, including any embodiment thereof, for use in a method of diagnosing a disease as subject to embodiment 202 will also be referred to herein as the second aspect of the present invention.

In accordance with the present invention, the compound of the present invention according to the first aspect of the present invention, including any embodiment thereof, for use in a method for the treatment of a disease as subject to embodiment 203 will also be referred to herein as the third aspect of the present invention.

In accordance with the present invention, the compound of the present invention according to the first aspect including any embodiment thereof, for use in a method for the identification of a subject, wherein the subject is likely to respond or likely not to respond to a treatment of a disease, wherein the method for the identification of a subject comprises carrying out a method of diagnosing a disease using a compound according to the first aspect of the present invention, including any embodiment thereof, as subject to embodiment 204 will also be referred to herein as the fourth aspect of the present invention.

In accordance with the present invention, the compound of the present invention according to the first aspect including any embodiment thereof, for use in a method for the selection of a subject from a group of subjects, wherein the subject is likely to respond or likely not to respond to a treatment of a disease, wherein the method for the selection of a subject from a group of subjects comprises carrying out a method of diagnosing a disease using a compound according to the first aspect of the present invention, including any embodiment thereof, as subject to embodiment 205 will also be referred to herein as the fifth aspect of the present invention.

In accordance with the present invention, the compound of the present invention according to the first aspect including any embodiment thereof, for use in a method for the stratification of a group of subjects into subjects which are likely to respond to a treatment of a disease, and into subjects which are not likely to respond to a treatment of a disease, wherein the method for the stratification of a group of subjects comprises carrying out a method of diagnosing a disease using a compound according to the first aspect of the present invention, including any embodiment thereof, as subject to embodiment 206 will also be referred to herein as the sixth aspect of the present invention.

In accordance with the present invention, the composition as subject to embodiment 216 comprising a compound according to the first aspect of the present invention, including any embodiment thereof, and a pharmaceutically acceptable excipient will also be referred to herein as the seventh aspect of the present invention.

In accordance with the present invention, the kit as subject to embodiment 217 comprising a compound according to the first aspect of the present invention, including any embodiment thereof, and one or more optional excipient(s) and optionally one or more device(s) will also be referred to herein as the eighth aspect of the present invention.

According to the present invention, each and any embodiment of the compound of the first aspect is also an embodiment of the peptide of the second aspect, and vice versa.

The present inventors have surprisingly found that the compounds of the invention show a high affinity to Gastric Inhibitory Peptide Receptor (GIPR). Furthermore, the present inventors have surprisingly found that compounds of the invention show other characteristics, which predestine the compounds of the invention for use in the diagnosis and therapy of diseases involving Gastric Inhibitory Peptide Receptor (GIPR). Such other characteristics comprise high stability in plasma and selectivity for Gastric Inhibitory Peptide Receptor (GIPR). Another surprising characteristic of the compounds of the invention is a low retention in normal, i.e. non-diseased tissues.

According to the present invention, the compounds of the invention may comprise one or more chelators, which is/are either directly or by means of a linker attached to the compounds of the invention. It is, however, preferred that the compounds of the invention comprise only one chelator. More preferably, such one chelator is attached to Xaa8 or such one chelator is comprised by the N-terminal group A. Most preferably, such one chelator is directly attached to Xaa8 without any linker.

In an embodiment and as preferably used herein, a diagnostically active compound is a compound, which is suitable for or useful in the diagnosis of a disease.

In an embodiment and as preferably used herein, a diagnostic agent or a diagnostically active agent is a compound, which is suitable for or useful in the diagnosis of a disease.

In an embodiment and as preferably used herein, a therapeutically active compound is a compound, which is suitable for or useful in the treatment of a disease.

In an embodiment and as preferably used herein, a therapeutic agent or a therapeutically active agent is a compound, which is suitable for or useful in the treatment of a disease.

In an embodiment and as preferably used herein, a theragnostically active compound is a compound, which is suitable for or useful in both the diagnosis and therapy of a disease.

In an embodiment and as preferably used herein, a theragnostic agent or a theragnostically active agent is a compound, which is suitable for or useful in both the diagnosis and therapy of a disease.

In an embodiment and as preferably used herein, theragnostics is a method for the combined diagnosis and therapy of a disease; preferably, the combined diagnostically and therapeutically active compounds used in theragnostics are radiolabeled.

In an embodiment and as preferably used herein, treatment of a disease is treatment and/or prevention of a disease.

In an embodiment and as preferably used herein, pEC50 is determined in a FACS binding assay, wherein the FACS binding assay is as described in the example part.

In an embodiment and as preferably used herein, pIC50 is determined in a FACS binding assay, wherein the FACS binding assay is as described in the example part.

In an embodiment and as preferably used herein, a disease involving GIPR is a disease, wherein cells including but not limited to tumor cells expressing or pancreatic beta cells, preferably in an upregulated manner, GIPR and tissue either expressing GIPR, preferably in an upregulated manner respectively, are either a or the cause for the disease and/or the symptoms of the disease, or are part of the pathology underlying the disease. A preferred GIPR-expressing cell is a tumor cell. In an embodiment of the disease, preferably when used in connection with the treatment, treating and/or therapy of the disease, affecting the cells, the tissue and pathology, respectively, results in cure, treatment or amelioration of the disease and/or the symptoms of the disease. In an embodiment of the disease, preferably when used in connection with the diagnosis and/or diagnosing of the disease, labeling of the GIPR-expressing cells and/or of the GIPR-expressing tissue allows discriminating or distinguishing said cells and/or said tissue from healthy or GIPR-non-expressing cells and/or healthy or GIPR-non-expressing tissue. More preferably such discrimination or distinction forms the basis for said diagnosis and diagnosing, respectively. In an embodiment thereof, labeling means the interaction of a detectable label either directly or indirectly with the GIPR-expressing cells and/or with the GIPR-expressing tissue or tissue containing such GIPR-expressing cells; more preferably such interaction involves or is based on the interaction of the label or a compound bearing such label with GIPR.

In an embodiment and as preferably used herein, a target cell is a cell, which is expressing GIPR and is a or the cause for a disease and/or the symptoms of a disease, or is part of the pathology underlying a disease.

In an embodiment and as preferably used herein, a non-target cell is a cell, which is either not expressing GIPR and/or is not a or the cause for a disease and/or the symptoms of a disease, or is part of the pathology underlying a disease.

In an embodiment and as preferably used herein, a neoplasm is an abnormal new growth of cells. The cells in a neoplasm grow more rapidly than normal cells and will continue to grow if not treated. A neoplasm may be benign or malignant.

In an embodiment and as preferably used herein, a tumor is a mass lesion that may be benign or malignant.

In an embodiment and as preferably used herein, a cancer is a malignant neoplasm.

In an embodiment and as preferably used herein, a cell and/or tissue expressing GIPR is overexpressing GIPR. More preferably, GIPR is overexpressed if the extent of expression is one observed in a cell and/or tissue of a person diagnosed to suffer from the disease, preferably the disease being cancer and metabolic disease as disclosed herein.

Accordingly, the compounds of the invention are thus particularly suitable for and useful in the diagnosis and treatment, respectively, of these diseases. Insofar, the above indications are indications, which can be treated by the compound of the invention. It will be understood by the person skilled in the art that also metastases and metastases of the above indications in particular can be treated and diagnosed by the compound of the invention and the methods of diagnosis and methods of treatment making use of the compound of the invention.

It is also within the present invention that the compound of the invention is used or is for use in a method for the treatment of a disease as disclosed herein. Such method, preferably, comprises the step of administering to a subject in need thereof a therapeutically effective amount of the compound of the invention. Such method includes, but is not limited to, curative or adjuvant cancer treatment. It is used as palliative treatment, wherein cure is not possible and the aim is for local disease control or symptomatic relief or as therapeutic treatment, wherein the therapy has survival benefit and it can be curative.

The method for the treatment of a disease as disclosed herein, includes the treatment of the disease disclosed herein, including tumors and cancer as well as metabolic diseases, and may be used either as the primary therapy or as second, third, fourth or last line therapy. It is also within the present invention to combine the compound of the invention with further therapeutic approaches. It is well known to the person skilled in the art that the precise treatment intent including curative, adjuvant, neoadjuvant, therapeutic, or palliative treatment intent will depend on the tumor type, location, and stage, as well as the general health of the patient.

Without wishing to be bound by any theory, the therapeutic effect of the compounds of present invention is based on the delivery of a radionuclide to a diseased GIPR expressing cell or structure, which is destroyed by the radiation emitted by the radionuclide. Preferably, GIPR is overexpressed by such cell or structure.

Without wishing to be bound by any theory, the therapeutic use of the compounds of the invention arises from the binding of said compounds to GIPR expressing cells, cancer cells in particular, wherein said cells are killed by the radiation emitted by the radionuclide. It will also be appreciated by a person skilled in the art that GIPR is a pan-neuroendocrine-tumor target.

Because of this, any respective cancer and tumor can be treated and diagnosed, respectively.

In an embodiment of the present invention, the disease is selected from the group comprising a cancer and tumor, respectively, expressing GIPR, preferably over-expressing GIPR. In a preferred embodiment, the cancer is a neuroendocrine cancer or neuroendocrine tumor, preferably each and any expressing or overexpressing GIPR.

In a further embodiment, the diseases is selected from the group comprising medullary thyroid cancer, gastrointestinal neuroendocrine neoplasms, ileal neuroendocrine neoplasms, pancreatic neuroendocrine neoplasms, nonfunctioning neuroendocrine neoplasms, Insulinomas, Gastrinomas, Glucagonomas, VIPomas, Somatostatinoma, ACTHoma, lung neuroendocrine neoplasms, typical carcinoid tumors, atypical carcinoid tumors, small cell carcinoma of the lung, large cell carcinoma of the lung, thymic neuroendocrine tumors, Merkel cell carcinoma, Pheochromocytoma of the adrenal gland, adrenal cancer, parathyroid cancer, paraganglioma, pituitary gland tumors, neuroendocrine tumors of the ovaries and neuroendocrine tumors of the testicles.

In a further embodiment, the disease is a metabolic disease. Preferably, the metabolic disease is one where GIPR is expressed, more preferably where GIPR is overexpressed in pancreatic beta cells. In a still further embodiment, the disease is selected from the group comprising metabolic diseases and disorders, preferably type 2 diabetes, type 1 diabetes, metabolic syndrome, insulin resistance, dyslipidemia, impaired fasting glucose, and impaired glucose tolerance.

In an embodiment, the compounds of the present invention are used to detect cells and tissues overexpressing the GIPR, whereby such detection is achieved by conjugating a detectable label to the compounds of the invention, preferably a detectable radionuclide. In a preferred embodiment, the cells and tissues detected are diseased cells and tissues and/or are either a or the cause for the disease and/or the symptoms of the disease, or are part of the pathology underlying the disease. In a further preferred embodiment, the diseased cells and tissues are causing and/or are part of an oncology indication (e.g. neoplasms, tumors, and cancers).

In another embodiment, the compounds of the present invention are used to treat cells and tissues overexpressing the GIPR. In a preferred embodiment, the cells and tissues treated are diseased cells and tissues and/or are either a or the cause for the disease and/or the symptoms of the disease, or are part of the pathology underlying the disease. In a further preferred embodiment, the diseased cells and tissues are causing and/or are part of an oncology indication (e.g. neoplasms, tumors, and cancers) and the therapeutic activity is achieved by conjugating therapeutically active effector to the compounds of the present invention, preferably a therapeutically active radionuclide.

An effective amount is a dosage of the compound sufficient to provide a therapeutically or medically desirable result or effect in the subject to which the compound is administered. The effective amount will vary with the particular condition being treated, the age and physical condition of the subject being treated, the severity of the condition, the duration of the treatment, the nature of the concurrent or combination therapy (if any), the specific route of administration and like factors within the knowledge and expertise of the health practitioner. For example, in connection with methods directed towards treating subjects having a condition characterized by abnormal cell proliferation, an effective amount to inhibit proliferation would be an amount sufficient to reduce or halt altogether the abnormal cell proliferation so as to slow or halt the development of or the progression of a cell mass such as, for example, a tumor. As used in the embodiments, “inhibit” embraces all of the foregoing.

In other embodiments, a therapeutically effective amount will be an amount necessary to extend the dormancy of micrometastases or to stabilize any residual primary tumor cells following surgical or drug therapy.

In a preferred embodiment, the compound of the present invention is for use in the treatment and/or prevention of a disease, whereby such treatment is targeted radionuclide therapy.

Targeted radionuclide therapy is a form of radiation therapy (also called radiotherapy) using molecules labeled with a radionuclide to deliver a toxic level of radiation to sites of disease.

Targeted radionuclide therapy may be applied systemically or locally. In contrast, in external beam radiation therapy a source outside of the body is producing a high-energy beam, which is then focused at sites of disease, passing through the skin into the body. It is as well distinguished from internal radiation therapy (brachytherapy), where a radioactive implant is placed at or near the site of disease.

Preferably, radionuclide therapy makes use of or is based on different forms of radiation emitted by a radionuclide. Such radiation can, for example, be any one of alpha (α), beta (β) or gamma (γ) radiation caused by the emission of photons, emission of electrons including but not limited to β⁻-particles and Auger-electrons, emission of protons, emission of neutrons, emission of positrons or emission of α-particles. Depending on the kind of particle or radiation emitted by said radionuclide, radionuclide therapy can, for example, be distinguished as β-particle radionuclide therapy, α-particle radionuclide therapy or Auger electron radionuclide therapy. All of these forms of radionuclide therapy are encompassed by the present invention, and all of these forms of radionuclide therapy can be realized by the compound of the invention, preferably under the proviso that the radionuclide attached to the compound of the invention, more preferably as an effector, is providing for this kind of radiation.

Radionuclide therapy preferably works by damaging the DNA of cells. The damage is caused by a β-particle, α-particle, or Auger electron directly or indirectly ionizing the atoms which make up the DNA chain. Indirect ionization happens as a result of the ionization of water, forming free radicals, notably hydroxyl radicals, which then damage the DNA.

In the most common forms of radionuclide therapy, most of the radiation effect is through free radicals. Because cells have mechanisms for repairing DNA damage, breaking the DNA on both strands proves to be the most significant technique in modifying cell characteristics. Because cancer cells generally are undifferentiated and stem cell-like, they reproduce more, and have a diminished ability to repair sub-lethal damage compared to most healthy differentiated cells. The DNA damage is inherited through cell division, accumulating damage to the cancer cells, causing them to die or reproduce more slowly.

Oxygen is a potent radiosensitizer, increasing the effectiveness of a given dose of radiation by forming DNA-damaging free radicals. Therefore, use of high-pressure oxygen tanks, blood substitutes that carry increased oxygen, hypoxic cell radiosensitizers such as misonidazole and metronidazole, and hypoxic cytotoxins, such as tirapazamine may be applied.

The total radioactive dose may be fractionated, i.e. spread out over time in one or more treatments for several important reasons. Fractionation allows normal cells time to recover, while tumor cells are generally less efficient in repair between fractions. Fractionation also allows tumor cells that were in a relatively radio-resistant phase of the cell cycle during one treatment to cycle into a sensitive phase of the cycle before the next fraction is given.

It is generally known that different cancers respond differently to radiation therapy. The response of a cancer to radiation is described by its radiosensitivity. Highly radiosensitive cancer cells are rapidly killed by modest doses of radiation. These include leukemias, most lymphomas, and germ cell tumors.

Radionuclide therapy is in itself painless. Many low-dose palliative treatments cause minimal or no side effects. Treatment to higher doses may cause varying side effects during treatment (acute side effects), in the months or years following treatment (long-term side effects), or after re-treatment (cumulative side effects). The nature, severity, and longevity of side effects depends on the organs that receive the radiation, the treatment itself (type of radionuclide, dose, fractionation, concurrent chemotherapy), and the patient.

It is within the present inventions that the method for the treatment of a disease of the invention may realize each and any of the above strategies, which are as such known in the art, and which insofar constitute further embodiments of the invention.

It is also within the present invention that the compound of the invention is used in a method for the diagnosis of a disease as disclosed herein. Such method, preferably, comprises the step of administering to a subject in need thereof a diagnostically effective amount of the compound of the invention.

In accordance with the present invention, an imaging method is selected from the group consisting of scintigraphy, Single Photon Emission Computed Tomography (SPECT) and Positron Emission Tomography (PET).

Scintigraphy is a form of diagnostic test or method used in nuclear medicine, wherein radiopharmaceuticals are internalized by cells, tissues and/or organs, preferably internalized in vivo, and radiation emitted by said internalized radiopharmaceuticals is captured by external detectors (gamma cameras) to form and display two-dimensional images. In contrast thereto, SPECT and PET forms and displays three-dimensional images. Because of this, SPECT and PET are classified as separate techniques to scintigraphy, although they also use gamma cameras to detect internal radiation. Scintigraphy is unlike a diagnostic X-ray where external radiation is passed through the body to form an image.

Single Photon Emission Tomography (SPECT) scans are a type of nuclear imaging technique using gamma rays. They are very similar to conventional nuclear medicine planar imaging using a gamma camera. Before the SPECT scan, the patient is injected with a radiolabeled compound emitting gamma rays that can be detected by the scanner. A computer collects the information from the gamma camera and translates this into two-dimensional cross-sections. These cross-sections can be added back together to form a three-dimensional image of an organ or a tissue. SPECT involves detection of gamma rays emitted singly, and sequentially, by the radionuclide provided by the radiolabeled compound. To acquire SPECT images, the gamma camera is rotated around the patient. Projections are acquired at defined points during the rotation, typically every 3-6 degrees. In most cases, a full 360 degree rotation is used to obtain an optimal reconstruction. The time taken to obtain each projection is also variable, but 15-20 seconds is typical. This gives a total scan time of 15-20 minutes. Multi-headed gamma cameras are faster. Since SPECT acquisition is very similar to planar gamma camera imaging, the same radiopharmaceuticals may be used.

Positron Emitting Tomography (PET) is a non-invasive, diagnostic imaging technique for measuring the biochemical, physiological and pathophysiological processes within the human body. PET is unique since it is able to produce images of the body's basic biochemistry or functions. Traditional diagnostic techniques, such as X-rays, CT scans, or MRI, produce images of the body's anatomy or structure. The premise with these techniques is that any changes in structure or anatomy associated with a disease can be seen. Biochemical and physiological processes are also altered by a disease, and may occur before any gross changes in anatomy. PET is an imaging technique that can visualize some of these early biochemical and physiological changes. PET scanners rely on radiation emitted from the patient to create the images. Each patient is given a minute amount of a radioactive compound that either closely resembles a natural substance used by the body or binds specifically to a receptor or molecular structure. As the radioisotope undergoes positron emission decay (also known as positive beta decay), it emits a positron, the antiparticle counterpart of an electron. After traveling up to a few millimeters, the positron encounters an electron and annihilates, producing a pair of annihilation (gamma) photons moving in opposite directions. These are detected when they reach a scintillation material in the scanning device, creating a burst of light, which is detected by photomultiplier tubes or silicon avalanche photodiodes. The technique depends on simultaneous or coincident detection of the pair of photons. Photons that do not arrive in pairs, i.e., within a few nanoseconds, are ignored. All coincidences are forwarded to the image processing unit where the final image data is produced using image reconstruction procedures.

SPECT/CT and PET/CT is the combination of SPECT and PET with computed tomography (CT). The key benefits of combining these modalities are improving the reader's confidence and accuracy. With traditional PET and SPECT, the limited number of photons emitted from the area of abnormality produces a very low-level background that makes it difficult to anatomically localize the area. Adding CT helps determine the location of the abnormal area from an anatomic perspective and categorize the likelihood that this represents a disease.

It is within the present inventions that the method for the diagnosis of a disease of the invention may realize each and any of the above strategies which are as such known in the art, and which insofar constitute further embodiments of the invention.

In an embodiment and as preferably used herein, a linkage is an attachment of two atoms of two independent moieties. A preferred linkage is a chemical bond or a plurality of chemical bonds. More preferably a chemical bond is a covalent bond or a plurality of chemical bonds. Most preferably the linkage is a covalent bond or a coordinate bond. As preferably used herein, an embodiment of a coordinate bond is a bond or group of bonds as realized when a metal is bound by a chelator. Depending on the type of atoms linked and their atomic environment different types of linkages are created. These types of linkage are defined by the type of atom arrangements created by the linkage.

For instance, the linking of a moiety comprising an amine with a moiety comprising a carboxylic acid leads to a linkage named amide (which is also referred to as amide linkage, —CO—N—, —N—CO—). It will be acknowledged by a person skilled in the art that this and the following examples of creating linkages are only prototypical examples and are by no means limiting the scope of the instant application. It will be acknowledged by a person in the art that the linking of a moiety comprising an isothiocyanate with a moiety comprising an amine leads to thiourea (which is also referred to as a thiourea linkage, —N—CS—N—), and linking of a moiety comprising a C atom with a moiety comprising a thiol-group (—C—SH) leads to thioether (which is also referred to as a thioether linkage, —C—S—C—). A non-limiting list of linkages as preferably used in connection with the chelator and linker of the invention and their characteristic type of atom arrangement is presented Table 2.

TABLE 2

| Linkage | Characteristic atom arrangement |
|---|---|
| Amide | 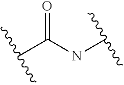 [SMILES: *NC(*)=O] |
| Sulfonamide | 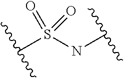 [SMILES: *NS(*)(=O)=O] |
| Urea | 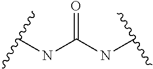 [SMILES: *NC(=O)N*] |
| Thioether | 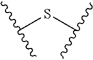 [SMILES: *S*] |
| Disulfide | 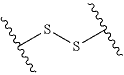 [SMILES: *SS*] |
| Ether | 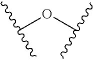 [SMILES: *O*] |
| Ester | 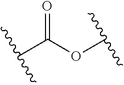 [SMILES: *OC(*)=O] |
| Carbamate | 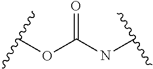 [SMILES: *NC(=O)O*] |
| Thiourea | 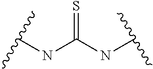 [SMILES: *NC(=S)N*] |
| Triazole | 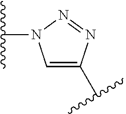 [SMILES: *c1cn(*)nn1] |

Examples of reactive groups which, in some embodiments of the invention, are used in the formation of linkages between the chelator and linker, directly between the chelator and the compound of the invention, the bio-distribution modifier and linker or the bio-distribution modifier and the compound of the invention are summarized in Table 3. It will, however, be understood by a person skilled in the art that neither the linkages which may be realized in embodiments for the formation of the conjugates of the invention are limited to the ones of Table 3 nor the reactive groups forming such linkages.

TABLE 3

| first | second | (type of) |
|---|---|---|
| reactive group | reactive group | linkage |
| amino | carboxylic acid | amide |
| amino | activated carboxylic acid | amide |
| amino | sulfonyl halide | sulfonamide |
| amino | isocyanate | urea |
| amino | p-nitrophenylcarbamate | urea |
| sulfhydryl | Michael acceptor (e.g. Maleimide) | thioether |
| bromo | sulfhydryl | thioether |
| isothiocyanate | amino | thiourea |
| hydroxyl | carboxylic acid | ester |
| azide | alkyne | triazole |
| sulfhydryl | sulfhydryl | disulfide |
| sulfhydryl | 2-Pyridine-disulfide | disulfide |
| carbonate ester | amino | carbamate |
| chloroformate | amino | carbamate |
| bromo | hydroxy | ether |

The following are reactive groups and functionalities which are utilized or amenable of forming linkages between moieties or structures as used in embodiments of the conjugate of the invention: Primary or secondary amino, carboxylic acid, activated carboxylic acid, chloro, bromo, iodo, sulfhydryl, hydroxyl, sulfonic acid, activated sulfonic acid, sulfonic acid esters like mesylate or tosylate, Michael acceptors, strained alkenes like trans cyclooctene, isocyanate, isothiocyanate, azide, alkyne and tetrazine.

As preferably used herein, the term “activated carboxylic acid” refers to a carboxylic acid group with the general formula —CO—X, wherein X is a leaving group. For example, activated forms of a carboxylic acid group may include, but are not limited to, acyl chlorides, symmetrical or unsymmetrical anhydrides, and esters. In some embodiments, the activated carboxylic acid group is an ester with pentafluorophenol, nitrophenol, benzotriazole, azabenzotriazole, thiophenol or N-hydroxysuccinimide (NHS) as leaving group.

In an embodiment and as preferably used herein the term “mediating a linkage” means that a linkage or a type of linkage is established, preferably a linkage between two moieties. In a preferred embodiment the linkage and the type of linkage is as defined herein.

To the extent it is referred in the instant application to a range indicated by a lower integer and a higher integer such as, for example, 1-4, such range is a representation of the lower integer, the higher integer and any integer between the lower integer and the higher integer. Insofar, the range is actually an individualized disclosure of said integer. In said example, the range of 1-4 thus means 1, 2, 3 and 4.

In an embodiment and as preferably used herein, the expression alkyl refers each and individually to a saturated, straight-chain or branched hydrocarbon group and is usually accompanied by a qualifier which specifies the number of carbon atoms it may contain. For example the expression (C₁-C₆)alkyl means each and individually any of methyl, ethyl, n-propyl, isopropyl, n-butyl, isobutyl, sec-butyl, tert-butyl, n-pentyl, 1-methyl-butyl, 1-ethyl-propyl, 3-methyl-butyl, 1,2-dimethyl-propyl, 2-methyl-butyl, 1,1-dimethyl-propyl, 2,2-dimethylpropyl, n-hexyl, 1,1-dimethyl-butyl and any other isoform of alkyl groups containing six saturated carbon atoms.

In an embodiment and as preferably used herein, (C₁-C₂)alkyl means each and individually any of methyl and ethyl.

In an embodiment and as preferably used herein, (C₁-C₃)alkyl means each and individually any of methyl, ethyl, n-propyl and isopropyl.

In an embodiment and as preferably used herein, (C₂-C₃)alkyl means each and individually any of ethyl, n-propyl and isopropyl.

In an embodiment and as preferably used herein, (C₁-C₄)alkyl means each and individually any of methyl, ethyl, n-propyl, isopropyl, n-butyl, isobutyl, sec-butyl and tert-butyl.

In an embodiment and as preferably used herein, (C₂-C₄)alkyl means each and individually any of ethyl, n-propyl, isopropyl, n-butyl, isobutyl, sec-butyl and tert-butyl.

In an embodiment and as preferably used herein, (C₁-C₆)alkyl means each and individually any of methyl, ethyl, n-propyl, isopropyl, n-butyl, isobutyl, sec-butyl, tert-butyl, n-pentyl, 2-pentyl, 2-methyl-butyl, 3-methyl-butyl, 3-pentyl, 3-methyl-but-2-yl, 2-methyl-but-2-yl, 2,2-dimethylpropyl, n-hexyl, 2-hexyl, 2-methyl-pentyl, 3-methyl-pentyl, 4-methyl-pentyl, 3-hexyl, 2-ethyl-butyl, 2-methyl-pent-2-yl, 2,2-dimethyl-butyl, 3,3-dimethyl-butyl, 3-methyl-pent-2-yl, 4-methyl-pent-2-yl, 2,3-dimethyl-butyl, 3-methyl-pent-3-yl, 2-methyl-pent-3-yl, 2,3-dimethyl-but-2-yl and 3,3-dimethyl-but-2-yl.

In an embodiment and as preferably used herein, (C₁-C₈)alkyl refers to a saturated or unsaturated, straight-chain or branched hydrocarbon group having from 1 to 8 carbon atoms. Representative (C₁-C₈)alkyl groups include, but are not limited to, any of methyl, ethyl, n-propyl, isopropyl, n-butyl, isobutyl, sec-butyl, tert-butyl, n-pentyl, 2-pentyl, 2-methyl-butyl, 3-methyl-butyl, 3-pentyl, 3-methyl-but-2-yl, 2-methyl-but-2-yl, 2,2-dimethylpropyl, n-hexyl, 2-hexyl, 2-methyl-pentyl, 3-methyl-pentyl, 4-methyl-pentyl, 3-hexyl, 2-ethyl-butyl, 2-methyl-pent-2-yl, 2,2-dimethyl-butyl, 3,3-dimethyl-butyl, 3-methyl-pent-2-yl, 4-methyl-pent-2-yl, 2,3-dimethyl-butyl, 3-methyl-pent-3-yl, 2-methyl-pent-3-yl, 2,3-dimethyl-but-2-yl, 3,3-dimethyl-but-2-yl, n-heptyl, 2-heptyl, 2-methyl-hexyl, 3-methyl-hexyl, 4-methyl-hexyl, 5-methyl-hexyl, 3-heptyl, 2-ethyl-pentyl, 3-ethyl-pentyl, 4-heptyl, 2-methyl-hex-2-yl, 2,2-dimethyl-pentyl, 3,3-dimethyl-pentyl, 4,4-dimethyl-pentyl, 3-methyl-hex-2-yl, 4-methyl-hex-2-yl, 5-methyl-hex-2-yl, 2,3-dimethyl-pentyl, 2,4-dimethyl-pentyl, 3,4-dimethyl-pentyl, 3-methyl-hex-3-yl, 2-ethyl-2-methyl-butyl, 4-methyl-hex-3-yl, 5-methyl-hex-3-yl, 2-ethyl-3-methyl-butyl, 2,3-dimethyl-pent-2-yl, 2,4-dimethyl-pent-2-yl, 3,3-dimethyl-pent-2-yl, 4,4-dimethyl-pent-2-yl, 2,2,3-trimethyl-butyl, 2,3,3-trimethyl-butyl, 2,3,3-trimethyl-but-2-yl, n-octyl, 2-octyl, 2-methyl-heptyl, 3-methyl-heptyl, 4-methyl-heptyl, 5-methyl-heptyl, 6-methyl-heptyl, 3-octyl, 2-ethyl-hexyl, 3-ethyl-hexyl, 4-ethyl-hexyl, 4-octyl, 2-propyl-pentyl, 2-methyl-hept-2-yl, 2,2-dimethyl-hexyl, 3,3-dimethyl-hexyl, 4,4-dimethyl-hexyl, 5,5-dimethyl-hexyl, 3-methyl-hept-2-yl, 4-methyl-hept-2-yl, 5-methyl-hept-2-yl, 6-methyl-hept-2-yl, 2,3-dimethyl-hex-1-yl, 2,4-dimethyl-hex-1-yl, 2,5-dimethyl-hex-1-yl, 3,4-dimethyl-hex-1-yl, 3,5-dimethyl-hex-1-yl, 3,5-dimethyl-hex-1-yl, 3-methyl-hept-3-yl, 2-ethyl-2-methyl-1-yl, 3-ethyl-3-methyl-1-yl, 4-methyl-hept-3-yl, 5-methyl-hept-3-yl, 6-methyl-hept-3-yl, 2-ethyl-3-methyl-pentyl, 2-ethyl-4-methyl-pentyl, 3-ethyl-4-methyl-pentyl, 2,3-dimethyl-hex-2-yl, 2,4-dimethyl-hex-2-yl, 2,5-dimethyl-hex-2-yl, 3,3-dimethyl-hex-2-yl, 3,4-dimethyl-hex-2-yl, 3,5-dimethyl-hex-2-yl, 4,4-dimethyl-hex-2-yl, 4,5-dimethyl-hex-2-yl, 5,5-dimethyl-hex-2-yl, 2,2,3-trimethyl-pentyl, 2,2,4-trimethyl-pentyl, 2,3,3-trimethyl-pentyl, 2,3,4-trimethyl-pentyl, 2,4,4-trimethyl-pentyl, 3,3,4-trimethyl-pentyl, 3,4,4-trimethyl-pentyl, 2,3,3-trimethyl-pent-2-yl, 2,3,4-trimethyl-pent-2-yl, 2,4,4-trimethyl-pent-2-yl, 3,4,4-trimethyl-pent-2-yl, 2,2,3,3-tetramethyl-butyl, 3,4-dimethyl-hex-3-yl, 3,5-dimethyl-hex-3-yl, 4,4-dimethyl-hex-3-yl, 4,5-dimethyl-hex-3-yl, 5,5-dimethyl-hex-3-yl, 3-ethyl-3-methyl-pent-2-yl, 3-ethyl-4-methyl-pent-2-yl, 3-ethyl-hex-3-yl, 2,2-diethyl-butyl, 3-ethyl-3-methyl-pentyl, 4-ethyl-hex-3-yl, 5-methyl-hept-3-yl, 2-ethyl-3-methyl-pentyl, 4-methyl-hept-4-yl, 3-methyl-hept-4-yl, 2-methyl-hept-4-yl, 3-ethyl-hex-2-yl, 2-ethyl-2-methyl-pentyl, 2-isopropyl-pentyl, 2,2-dimethyl-hex-3-yl, 2,2,4-trimethyl-pent-3-yl and 2-ethyl-3-methyl-pentyl. A (C₁-C₈)alkyl group can be unsubstituted or substituted with one or more groups, including, but not limited to, (C₁-C₈)alkyl, —O—[(C₁-C₈)alkyl], -aryl, —CO—R′, —O—CO—R′, —CO—OR′, —CO—NH₂, —CO—NHR′, —CO—NR′₂, —NH—CO—R′, —SO₂—R′, —SO—R′, —OH, -halogen, —N3, —NH₂, —NHR′, —NR′₂ and —CN; where each R′ is independently selected from —(C₁-C₈)alkyl and aryl.

In an embodiment, and as preferably used herein, “nucleophile” refers to a chemical species capable of forming a chemical bond by donating an electron pair. In a more preferred embodiment and as preferably used herein a nucleophile comprises a heteroatom, wherein the heteroatom preferably comprises a nitrogen atom or a sulfur atom.

In an embodiment and as preferably used herein, “bis-nucleophile” refers to a group comprising a first nucleophile and a second nucleophile, wherein each nucleophile of the bis-nucleophile comprises a heteroatom. The heteroatom of the first of the two nucleophiles is a nitrogen atom. The heteroatom of the second of the two nucleophiles is selected from the group comprising a nitrogen atom and a sulfur atom, preferably the heteroatom of the second of the two nucleophiles is a sulfur atom. In a more preferred embodiment the “bis-nucleophile” is an aliphatic “bis-nucleophile”. In an aliphatic “bis-nucleophile” the heteroatoms of the nucleophiles are covalently attached to the terminal carbon atoms of a (C₁-C₄)alkyl, preferably a (C₁-C₃)alkyl and more preferably a (C₁-C₂)alkyl, wherein each carbon atom optionally can be substituted with one or more groups, preferably one group, wherein said group is individually and independently selected from the group comprising —CONH₂, —CONHR, —COOH, and —CH₂OH. Examples of bis-nucleophiles include, but are not limited to, 2-aminoethanethiol, 2-aminoethanol, ethylenediamine, 3-aminopropane-1-thiol, 3-amino-1-propanol and 1,3-diaminopropane.

In an embodiment and as preferably used herein, “aryl” or “aryl ring” refers to a carbocyclic aromatic group, wherein the carbocyclic aromatic group is selected from the group consisting of aromatic groups with a single ring and aromatic groups with two or more fused rings, preferably two rings. Examples of carbocyclic aromatic groups with a single benzene ring include, but are not limited to, phenyl, and examples of carbocyclic aromatic groups with two or more fused benzene rings include, but are not limited to naphthyl and anthracenyl. A carbocyclic aromatic group can be unsubstituted or substituted with one or more groups including, but not limited to, —(C₁-C₈)alkyl, —O—[(C₁-C₈)alkyl], -aryl, —CO—R′, —O—CO—R′, —CO—OR′, —CO—NH₂, —CO—NHR′, —CO—NR′₂, —NH—CO—R′, —SO₂—R′, —SO—R′, —OH, -halogen, —N₃, —NH₂, —NHR′, —NR′₂ and —CN; where each R′ is independently selected from —(C₁-C₈)alkyl and aryl. In a more preferred embodiment the carbocyclic aromatic group comprises two substitutents of the one or more groups, wherein the first of said two groups is attached to a first ring atom of the carbocyclic aromatic group. The second of said two groups is attached either to a second ring atom of the carbocyclic aromatic group directly adjacent to said first ring atom or to a second ring atom of the carbocyclic aromatic group, wherein the second ring atom is separated from the first ring atom by a further ring atom.

In an embodiment and as preferably used herein, “heteroaryl” or “heteroaryl ring” refers to a heterocyclic aromatic group, wherein the heterocyclic aromatic group is selected from the group consisting of heterocyclic aromatic groups with a single ring and heterocyclic aromatic group with two or more fused rings, preferably two rings. Examples of heterocyclic aromatic groups with a single ring include, but are not limited to, furanyl, thiophenyl, pyrroyl, pyrazoyl, oxazoyl, thiazoyl, imidazoyl, pyridinyl pyrimidinyl and examples of heterocyclic aromatic groups with two or more fused rings include, but are not limited to benzothiophenyl, benzofuranyl, indoyl and quinolinyl. A heterocyclic aromatic group can be unsubstituted or substituted with one or more groups including, but not limited to, —(C₁-C₈)alkyl, —O—[(C₁-C₈)alkyl], -aryl, —CO—R′, —O—CO—R′, —CO—OR′, —CO—NH₂, —CO—NHR′, —CO—NR′₂, —NH—CO—R′, —SO₂—R′, —SO—R′, —OH, -halogen, —N₃, —NH₂, —NHR′, —NR′₂ and —CN, wherein each R′ is independently selected from —(C₁-C₈)alkyl and aryl. In a more preferred embodiment the heterocyclic aromatic group comprises two substitutents of the one or more groups, wherein the first of said two groups is attached to a first ring atom of the heterocyclic aromatic group. The second of said two groups is attached either to a second ring atom of the heterocyclic aromatic group directly adjacent to said first ring atom or to a second ring atom of the heterocyclic aromatic group, wherein the second ring atom is separated from the first ring atom by a further ring atom.

In an embodiment and as preferably used herein, “aromatic residue” or/and “aromatic ring” refers to an aryl as defined herein.

In an embodiment and as preferably used herein, “aromatic side chain” refers to a side chain wherein the side chain comprises an “aromatic ring” as defined herein.

In an embodiment and as preferably used herein, “bicyclic aromatic ring” refers to an aryl as defined herein, wherein the carbocyclic aromatic group of the aryl is selected from the group of aromatic groups with two or more fused rings, preferably two rings.

In an embodiment and as preferably used herein, “bicyclic aromatic side chain” refers to a side chain wherein the side chain comprises a “bicyclic aromatic ring” as defined herein.

In an embodiment and as preferably used herein, “(C₉-C₁₀)bicyclic aromatic ring” refers to a “bicyclic aromatic ring” comprising 9 or 10 atoms forming the two rings of the bicycle as defined herein.

In an embodiment and as preferably used herein, “(C₉-C₁₀)bicyclic aromatic side chain” refers to a side chain, wherein the side chain comprises a “(C₉-C₁₀)bicyclic aromatic ring” as defined herein.

In an embodiment and as preferably used herein, “bicyclic non-aromatic residue” refers to a bicyclic heterocyclic group with two fused cycles, wherein at least one atom is different from carbon, preferably the at least on atom is nitrogen, sulfur or oxygen. Examples of bicyclic non-aromatic residue include, but are not limited to, octahydroquinoline and octahydroisoquinoline. A bicyclic non-aromatic residue can be unsubstituted or substituted with one or more groups including, but not limited to, —(C₁-C₈)alkyl, —O—[(C₁-C₈)alkyl], -aryl, —CO—R′, —O—CO—R′, —CO—OR′, —CO—NH₂, —CO—NHR′, —CO—NR′₂, —NH—CO—R′, —SO₂—R′, —SO—R′, —OH, -halogen, —N₃, —NH₂, —NHR′, —NR′₂ and —CN; where each R′ is independently selected from —(C₁-C₈)alkyl and aryl.

In an embodiment and as preferably used herein, “(C₉-C₁₀)bicyclic non-aromatic residue” refers to a “bicyclic non-aromatic residue” as defined herein, comprising 9 or 10 atoms forming the two rings of the bicycle.

In an embodiment and as preferably used herein, “heteroaromatic side chain” refers to a side chain wherein the side chain comprises a heteroaryl as defined herein.

In an embodiment and as preferably used herein, “bicyclic heteroaromatic ring” refers to a heteroaryl as defined herein, wherein the heteroaryl comprises two rings.

In an embodiment and as preferably used herein, “bicyclic heteroaromatic side chain” refers to a side chain, wherein the side chain comprises a “bicyclic heteroaromatic ring” as defined herein.

In an embodiment and as preferably used herein, “(C₅-C₆)heteroaryl” refers to a heteroaryl as defined herein, wherein the heterocyclic aromatic group of the heteroary is selected from the group consisting of heterocyclic aromatic groups with a single ring.

In an embodiment and as preferably used herein, “(C₅-C₆)heteroaromatic side chain” refers to a side chain, wherein the side chain comprises a “(C₅-C₆)heteroaryl” as defined herein.

In an embodiment and as preferably used herein, “(C₉-C₁₀)bicyclic heteroaromatic ring” refers to a refers to a heteroaryl as defined herein, wherein the heterocyclic aromatic group of the heteroaryl is selected from the group of heterocyclic aromatic groups with two rings, wherein the heterocyclic aromatic group with two rings comprises 9 to 10 ring atoms.

In an embodiment and as preferably used herein, “(C₉-C₁₀)bicyclic heteroaromatic side chain” refers to a side chain comprising a “(C₉-C₁₀)bicyclic heteroaromatic ring” as defined herein.

In an embodiment and as preferably used herein, arylene refers to an aryl group which has two covalent bonds and can be in the ortho, meta, or para configurations as shown in the following structures:

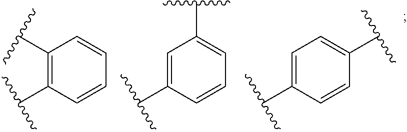

in which the phenyl group can be unsubstituted or substituted with four groups, including, but not limited to, (C₁-C₈)alkyl, —O—[(C₁-C₈)alkyl], -aryl, —CO—R′, —O—CO—R′, —CO—OR′, —CO—NH₂, —CO—NHR′, —CO—NR′₂, —NH—CO—R′, —SO₂—R′, —SO—R′, —OH, -halogen, —N₃, —NH₂, —NHR′, —NR′₂ and —CN; where each R′ is independently selected from —(C₁-C₈)alkyl and aryl.

In an embodiment and as preferably used herein, the C-terminal group C-term is one amino acid residue or is a dipeptide comprising two amino acid residues. If C-term is the dipeptide, the first of the two amino acid residues is the one at the N-terminus (assuming that the two amino acid residues are covalently attached to each other by a peptide bond) and the last of the two amino acid residues is the one at the C-terminus. The one or first of the two amino acid is covalently attached to the C-terminal amino acid residue of the compound of invention. In a more specific embodiment and as preferably used herein, an optional linker is interspersed between the C-terminal amino acid residue of the compound of invention and the one amino acid residue or the first amino acid residue of the C-terminal dipeptide.

In an embodiment and as preferably used herein, atoms with unspecified atomic mass numbers in any structural formula or in any passage of the instant specification including the claims are either of unspecified isotopic composition, naturally occurring mixtures of isotopes or individual isotopes. This applies in particular to carbon, oxygen, nitrogen, sulfur, phosphorus, halogens and metal atoms, including but not limited to C, O, N, S, F, P, Cl, Br, At, Sc, Cr, Mn, Co, Fe, Cu, Ga, Sr, Zr, Y, Mo, Tc, Ru, Rh, Pd, Pt, Ag, In, Sb, Sn, Te, I, Pr, Pm, Dy, Sm, Gd, Tb, Ho, Dy, Er, Yb, Tm, Lu, Sn, Re, Rd, Os, Ir, Au, Pb, Bi, Po, Fr, Ra, Ac, Th and Fm.

In an embodiment and as preferably used herein, a chelator is a compound which is capable of forming a chelate, whereby a chelate is a compound, preferably a cyclic compound where a metal or a moiety having an electron gap or a lone pair of electrons participates in the formation of the ring. More preferably, a chelator is a kind of compound where a single ligand occupies more than one coordination site at a central atom.

In an embodiment and as preferably used herein, a bio-distribution modifier is a compound the function of which can be the attenuation of unintended or undesired bio-distribution of the compound of the invention by altering physio-chemical properties of the compound of the invention. Alternatively or additionally, the function of the bio-distribution modifier can be the binding to components of the serum thus extending the serum half-life and/or the alteration of serum half-life by modulating renal clearance.

In an embodiment and as preferably used herein, “dicarboxylic acid residue” refers to a compound with two carboxylic acid groups, wherein the two carboxylic groups are attached to the terminal carbon atoms of a (C₁-C₆)alkyl. Examples of dicarboxylic acid resides residue include, but are not limited to, malonic acid, succinic acid, glutaric acid and pimelic acid.

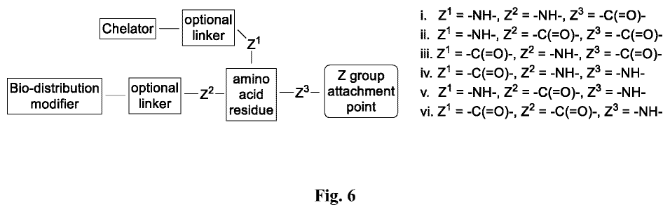

In an embodiment and as preferably used herein, “Z group” refers to (a) a chelator optionally comprising a linker, (b) a bio-distribution modifier optionally comprising a linker, or (c) a combination of a chelator optionally comprising a linker and a bio-distribution modifier optionally comprising a linker, whereby in such combination the chelator and the bio-distribution modifier are covalently linked to each other by an amino acid residue. Said amino acid residue (i) either directly links the chelator to the bio-distribution modifier, (ii) links the chelator comprising a linker to the bio-distribution modifier, (iii) links the chelator to the bio-distribution modifier comprising a linker, or (iv) links the chelator comprising a linker to the bio-distribution modifier comprising a linker. In a preferred embodiment, the amino acid residue linking the chelator optionally comprising a linker and the bio-distribution modifier optionally comprising a linker, also covalently attaches the combination of the chelator optionally comprising a linker and the bio-distribution modifier optionally comprising a linker to a group and position, respectively, of the compound of the invention where a Z group may be covalently attached. The group and position, respectively, of the compound of the invention where a Z group may be covalently attached, is also referred to herein as Z group attachment point. FIG. 6 illustrates various options for attaching the combination of the chelator optionally comprising a linker and the bio-distribution modifier optionally comprising a linker to a Z group attachment point.

In accordance with the present invention, Z group attachment points are in particular the following ones: the N-terminal modification group A, which may comprise both a first Z group and a second Z group; Xaa4 comprising one Z group; Xaa5 comprising one Z group; Xaa8 comprising one Z group; Yc comprising one Z group; and the C-terminal modification group C-term, which may comprise both a first and a second Z-group.

More preferred Z group attachment points are the N-terminal modification group A comprising one Z-group; Xaa8 comprising one Z group; and the C-terminal modification group C-term comprising one Z-group.

As will be appreciated by a person skilled in the art, the chemical nature of the chelator, of the bio-distribution modifier, of the linker of the chelator and of the linker of the bio-distribution modifier define the kind of amino acid residue to be used for attaching any one of them to the compound of the invention. Typically, the chelator, the bio-distribution modifier, the linker of the chelator and of the linker of the bio-distribution comprise a chemical group suitable for reacting with the amino acid residue, whereby such chemical group is selected form the group comprising a carboxylic acid group and an amino group. In order for the amino acid residue to be able to (a) form the combination of a chelator optionally comprising a linker and a bio-distribution modifier optionally comprising a linker, and (b) covalently link such combination to a Z group attachment point, the amino acid reside has to comprise at least three functional groups. Typically, the amino acid residue comprises (a) two carboxylic acid groups and one amino group or (b) one carboxylic acid group and two amino groups. Preferred amino acid residues are residues of an amino acid selected from the group comprising Lys, lys, Glu and glu.

In an embodiment and as preferably used herein, “amino acid residue” refers to all atoms of an amino acid, which remain in a peptide chain after the combination of said amino acid with another amino acid, an N-terminal modification group A, a C-terminal modification group C-term or a Z group either to either the α-nitrogen atom of said amino acid or the carbonyl group of said amino acid.

In an embodiment and as preferably used herein, “L-configuration” and “D-configuration” refer to the stereo configuration of the α-carbon atom of an α-amino acid or a residue of an α-amino acid.

In an embodiment and as preferably used herein, “side chain” refers to all atoms of an amino acid residue that are not comprised in the “main chain” portion of said amino acid residue. “Main chain” refers to the structure that is formed by the consecutive connection of at least a first amino acid and a second amino acid, whereby the α-nitrogen atom of an α-amino acid being the second amino acid, the β-nitrogen atom of a β-amino acid being the second amino acid, the γ-nitrogen of a γ-amino acid being the second amino acid, the δ-nitrogen atom of a δ-amino acid being the second amino acid, the ε-nitrogen of an ε-amino acid being the second amino acid or the ω-nitrogen of an ω-amino acid being the second amino acid is connected or covalently linked to the C-1 carbonyl group of a preceding amino acid being the first amino acid.

In an embodiment and as preferably used herein, the term “linking”, “linked” or “link” refers to or describes a scenario, where two or more moieties are connected either directly or indirectly with an interspersed group. Preferably, such moiety is a chelator, an amino acid residue or a bio-distribution modifier. In a more preferred embodiment and as preferably used herein, such interspersed group is a linker. In another more preferred embodiment, a residue of an amino acid is linking two moieties, wherein said residue of an amino acid is part of a Z-group, wherein the Z group comprises a combination of a chelator optionally comprising a linker and a bio-distribution modifier optionally comprising a linker the amino acid residue connects the chelator and the bio-distribution modifier.

In an embodiment and as preferably used herein, the term “interspersed” refers to or describes a scenario in which a group, preferably the group is a linker, is inserted between a first moiety and a second moiety. Such moiety for example is an amino acid residue, a chelator or a bio-distribution modifier. The inserted group is covalently attached to both of the moieties.

Compounds of the invention typically contain amino acid sequences as provided herein. Conventional amino acids, also referred to as natural amino acids are identified according to their standard three-letter codes, as set forth in Table 4.

TABLE 4
Conventional amino acids and their abbreviations

| 3-letter codes | Amino acid |
|---|---|
| Ala | Alanine |
| Cys | Cysteine |
| Asp | Aspartic acid |
| Glu | Glutamic acid |
| Phe | Phenylalanine |
| Gly | Glycine |
| His | Histidine |
| Ile | Isoleucine |
| Lys | Lysine |
| Leu | Leucine |
| Met | Methionine |
| Asn | Asparagine |
| Pro | Proline |
| Gln | Glutamine |
| Arg | Arginine |
| Ser | Serine |
| Thr | Threonine |
| Val | Valine |
| Trp | Tryptophan |
| Tyr | Tyrosine |

Non-conventional amino acids, also referred to as non-natural amino acids, are any kind of non-oligomeric compound, which comprises an amino group and a carboxylic group and is not a conventional amino acid.

Examples of conventional amino acids, non-conventional amino acids and other building blocks as used for the construction compounds of the invention are identified according to their abbreviation or name found in Table 5. The structures of some building blocks are depicted as residues of the corresponding amino acids (amino acid residues) or these building blocks are shown as residue which is completely attached to another structure like a peptide or amino acid residue. The structures of the amino acids shown as residues of the amino acids are shown how they are presented after implementation in the peptide sequence and not as explicit amino acids.

Furthermore, residues of building blocks such as bis-nucleophiles and dicarboxylic acids are shown as how they are presented after implementation in the compounds of the invention. Furthermore, heteroaryls, aryls and heterocyclic rings as elements of peptidic macrocycles are shown as how they are presented after implementation in the compounds of the invention. Furthermore, residues of structures of blocking groups usually located at the N-terminus of a peptide (e.g., Ac) or structures of blocking groups usually located of the C-terminus of a peptide (e.g., NH₂ or OH) are shown as how they are presented after implementation in the peptide sequence. Some larger chemical moieties consisting of more than one moiety are also shown for the reason of clarity (e.g. Apc(DOTA), glu(AGLU) or Apc(DOTA-glu(AGLU))).

TABLE 5
Abbreviation, name and structure of non-natural amino-acid and other building blocks and chemical moieties

| Abbrevi- | | | |
|---|---|---|---|
| ation | Name | Residue | Building Block |
| 1Ni | 3- (1-naphthyl)- L- alanine | 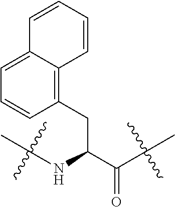 [SMILES: *N[C@@H](Cc1cccc2ccccc12)C(*)=O] | 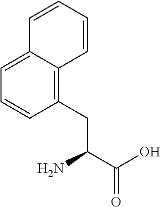 [SMILES: N[C@@H](Cc1cccc2ccccc12)C(=O)O] |
| 2Lut | 2,6- lutidylidene (derived from 2,6- lutidine) | 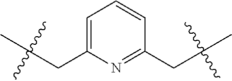 [SMILES: *Cc1cccc(C*)n1] | |
| 2MeBn | 2- Methyl- benzylidene (derived from 1,2- Xylene) | 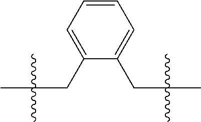 [SMILES: *Cc1ccccc1C*] | |
| 2Ni | 3-(2- naphthyl)-L- alanine | 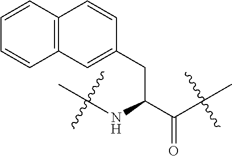 [SMILES: *N[C@@H](Cc1ccc2ccccc2c1)C(*)=O] | 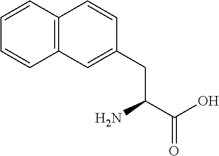 [SMILES: N[C@@H](Cc1ccc2ccccc2c1)C(=O)O] |
| 3CbBn | 3- Carboxy- benzyl (derived from meta-methyl benzoic acid) | 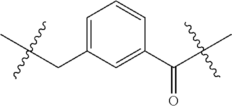 [SMILES: *Cc1cccc(C(*)=O)c1] | |
| 3Lut | 3,5- lutidylidene (derived from 3,5- lutidine) | 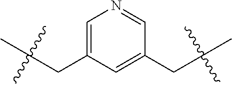 [SMILES: *Cc1cncc(C*)c1] | |
| 3MeBn | 3- Methyl- benzylidene (derived from 1,3-Xylene) | 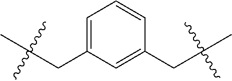 [SMILES: *Cc1cccc(C*)c1] | |
| 3MeBz | 3-methyl- benzoyl (derived from meta-methyl benzoic acid) | 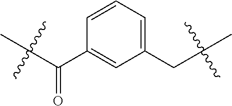 [SMILES: *Cc1cccc(C(*)=O)c1] | |
| 3Mh | 3-Methyl-L- histidine | 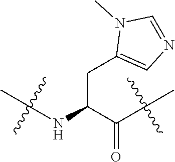 [SMILES: *N[C@@H](Cc1cncn1C)C(*)=O] | 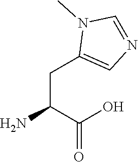 |
| 4Ap | 4-(S)- Amino-L- proline | 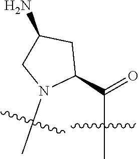 [SMILES: *C(=O)[C@@H]1C[C@H](N)CN1*] | 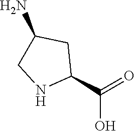 |
| 4ClPhp | 4- chlorophenyl propionic acid | 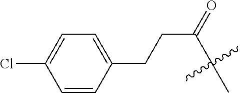 [SMILES: *C(=O)CCc1ccc(Cl)cc1] | 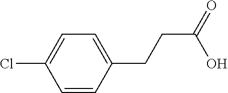 [SMILES: O=C(O)CCc1ccc(Cl)cc1] |
| 4Dfp | 4,4- Difluoro-L- proline | 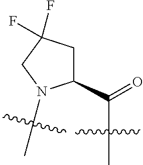 [SMILES: *C(=O)[C@@H]1CC(F)(F)CN1*] | 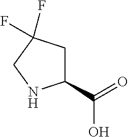 |
| 4FPhp | 4- fluorophenyl propionic acid | 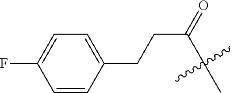 [SMILES: *C(=O)CCc1ccc(F)cc1] | 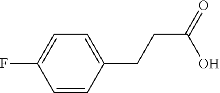 [SMILES: O=C(O)CCc1ccc(F)cc1] |
| 4MeBn | 4- Methyl- benzylidene (derived from 1,4- Xylene) | 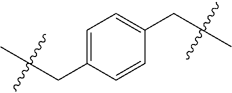 [SMILES: *Cc1ccc(C*)cc1] | |
| 4OHPhp | 4- hydroxy- phenyl propionic acid | 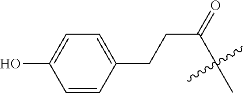 [SMILES: *C(=O)CCc1ccc(O)cc1] | 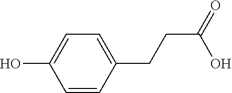 [SMILES: O=C(O)CCc1ccc(O)cc1] |
| 4Tfp | 4-(R)- Fluoro-L- proline | 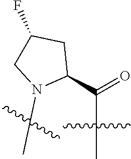 [SMILES: *C(=O)[C@@H]1C[C@@H](F)CN1*] | 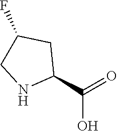 |
| 5Brw | 5-bromo-L- tryptophan | 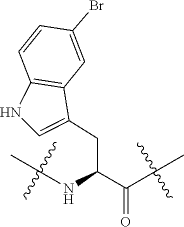 [SMILES: *N[C@@H](Cc1c[nH]c2ccc(Br)cc12)C(*)=O] | 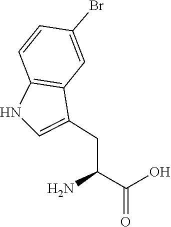 [SMILES: N[C@@H](Cc1c[nH]c2ccc(Br)cc12)C(=O)O] |
| 5Clw | 5-chloro-L- tryptophan | 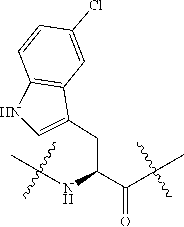 [SMILES: *N[C@@H](Cc1c[nH]c2ccc(Cl)cc12)C(*)=O] | 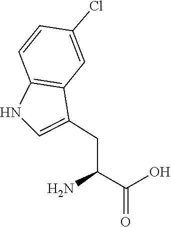 [SMILES: N[C@@H](Cc1c[nH]c2ccc(Cl)cc12)C(=O)O] |
| 5Fw | 5-fluoro-L- tryptophan | 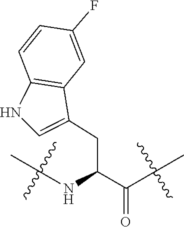 [SMILES: *N[C@@H](Cc1c[nH]c2ccc(F)cc12)C(*)=O] | 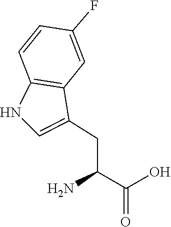 [SMILES: N[C@@H](Cc1c[nH]c2ccc(F)cc12)C(=O)O] |
| 6Clw | 6-chloro-L- tryptophan | 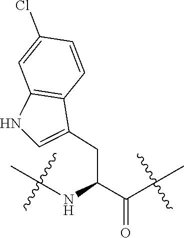 [SMILES: *N[C@@H](Cc1c[nH]c2cc(Cl)ccc12)C(*)=O] | 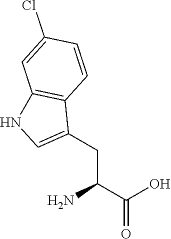 [SMILES: N[C@@H](Cc1c[nH]c2cc(Cl)ccc12)C(=O)O] |
| 6Fw | 6-Fluoro-L- tryptophan | 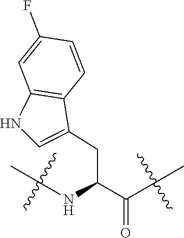 [SMILES: *N[C@@H](Cc1c[nH]c2cc(F)ccc12)C(*)=O] | 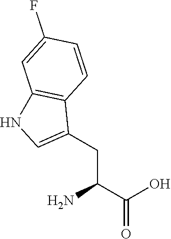 [SMILES: N[C@@H](Cc1c[nH]c2cc(F)ccc12)C(=O)O] |
| 7Fw | 7-fluoro-L- tryptophan | 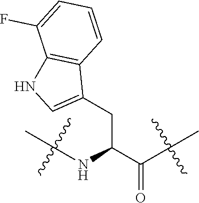 [SMILES: *N[C@@H](Cc1c[nH]c2c(F)cccc12)C(*)=O] | 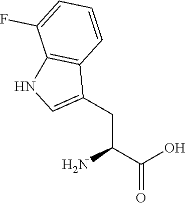 [SMILES: N[C@@H](Cc1c[nH]c2c(F)cccc12)C(=O)O] |
| 7Nw | 7-aza-L- tryptophan | 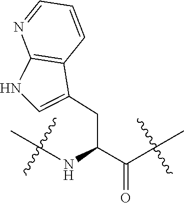 [SMILES: *N[C@@H](Cc1c[nH]c2ncccc12)C(*)=O] | 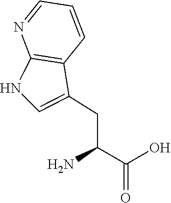 [SMILES: N[C@@H](Cc1c[nH]c2ncccc12)C(=O)O] |
| Ac | Acetyl | 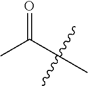 [SMILES: *C(C)=O] | 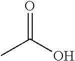 |
| Abu | 2S- aminobutyric acid | 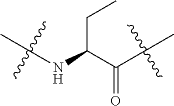 [SMILES: *N[C@@H](CC)C(*)=O] | 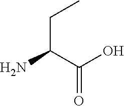 |
| AET | 2- aminoethane- 1- thiol | 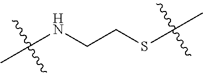 [SMILES: *NCCS*] | 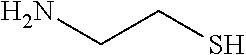 |
| AETO | 2- amino- ethane-1- thiol oxidized | 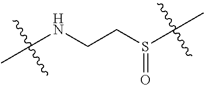 [SMILES: *NCCS(*)=O] | |
| ABOL | 4- Amino- butane- 1,2,3-triol | 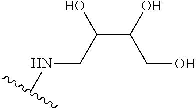 [SMILES: *NCC(O)C(O)CO] | 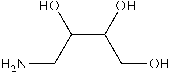 |
| AHOL | 6-Amino- hexane- 1,2,3,4,5- pentaol | 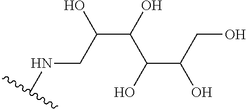 [SMILES: *NCC(O)C(O)C(O)C(O)CO] | 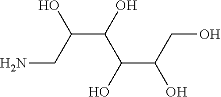 [SMILES: NCC(O)C(O)C(O)C(O)CO] |
| AGLU | 1-amino- 1-deoxy- D-glucitol | 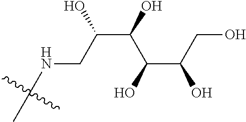 [SMILES: *NC[C@H](O)[C@@H](O)[C@H](O)[C@H](O)CO] | 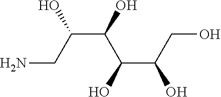 [SMILES: NC[C@H](O)[C@@H](O)[C@H](O)[C@H](O)CO] |
| Aib | 1-amino- isobutyric acid | 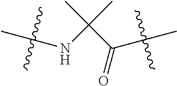 [SMILES: *NC(C)(C)C(*)=O] | 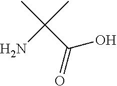 |
| Aic | 2- Amino- indane- 2- carboxylic acid | 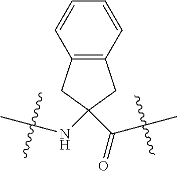 [SMILES: *NC1(C(*)=O)Cc2ccccc2C1] | 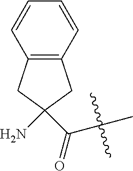 [SMILES: *C(=O)C1(N)Cc2ccccc2C1] |
| ala | D-alanine | 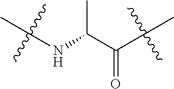 [SMILES: *N[C@H](C)C(*)=O] | 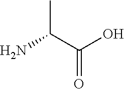 |
| Ala | L-alanine | 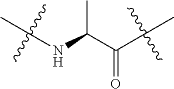 [SMILES: *N[C@@H](C)C(*)=O] | 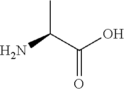 |
| alloThr | L-allo- threonine | 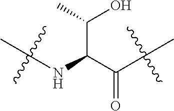 [SMILES: *N[C@H](C(*)=O)[C@H](C)O] | 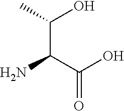 |
| Aml | α-methyl- L-leucine | 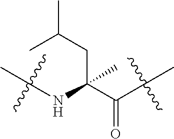 | 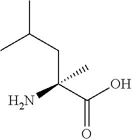 |
| Ams | α-methyl- L-serine | 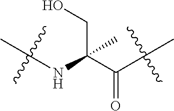 | 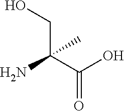 |
| APAc | 4-amino-1- carboxy- methyl- piperidine | 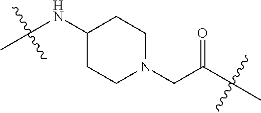 [SMILES: *NC1CCN(CC(*)=O)CC1] | 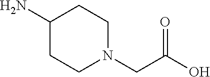 [SMILES: NC1CCN(CC(=O)O)CC1] |
| Apc | 4-amino- piperidine- 4- carboxylic acid | 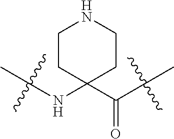 [SMILES: *NC1(C(*)=O)CCNCC1] | 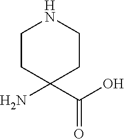 |
| Apc (DOTA) | 4- amino- piperidine- 4-carboxylic acid DOTA conjugate | 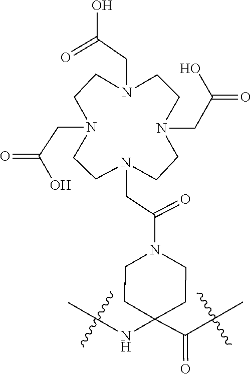 [SMILES: *NC1(C(*)=O)CCN(C(=O)CN2CCN(CC(=O)O)CCN(CC(=O)O)CCN(CC(=O)O)CC2)CC1] | |
| Apc (DOTA- glu (AGLU)) | 4-amino- piperidine- 4-carboxylic acid (glu(DOTA/ AGLU)) conjugate | 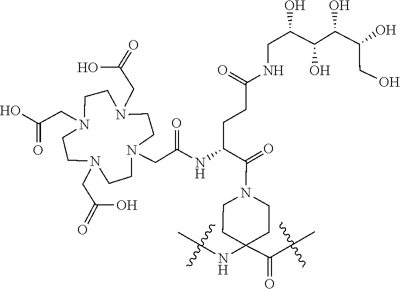 [SMILES: *NC1(C(*)=O)CCN(C(=O)[C@@H](CCC(=O)NC[C@H](O)[C@@H](O)[C@H](O)[C@H](O)CO)NC(=O)CN2CCN(CC(=O)O)CCN(CC(=O)O)CCN(CC(=O)O)CC2)CC1] | |
| Ape | 1,5-diamine- pentane | 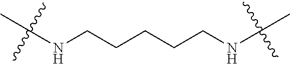 [SMILES: *NCCCCCN*] | 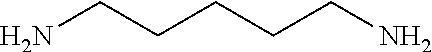 |
| APOL | 5-Amino- pentane- 1,2,3,4- tetraol | 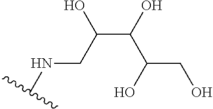 [SMILES: *NCC(O)C(O)C(O)CO] | 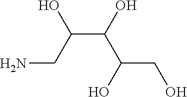 |
| APrOL | 3-Amino- propane- 1,2-diol | 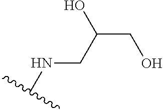 [SMILES: *NCC(O)CO] | 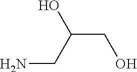 |
| Arg | L-arginine | 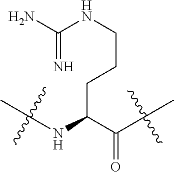 [SMILES: *N[C@@H](CCCNC(=N)N)C(*)=O] | 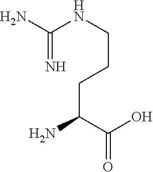 [SMILES: N=C(N)NCCC[C@H](N)C(=O)O] |
| Asn | L- asparagine | 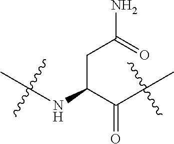 [SMILES: *N[C@@H](CC(N)=O)C(*)=O] | 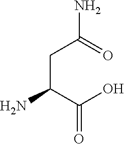 |
| asp | D-aspartic acid | 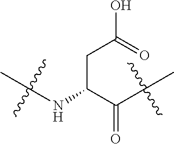 [SMILES: *N[C@H](CC(=O)O)C(*)=O] | 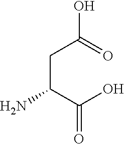 |
| Asp | L-aspartic acid | 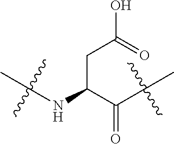 [SMILES: *N[C@@H](CC(=O)O)C(*)=O] | 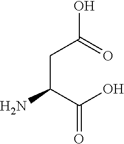 |
| Aze | (S)- Azetidine-2- carboxylic acid | 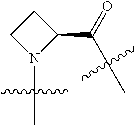 [SMILES: *C(=O)[C@@H]1CCN1*] | 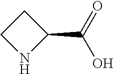 |
| Bhf | (S)-β- Homophenyl alanine | 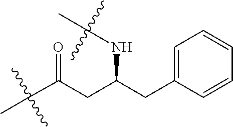 [SMILES: *N[C@H](CC(*)=O)Cc1ccccc1] | 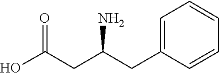 [SMILES: N[C@H](CC(=O)O)Cc1ccccc1] |
| Bio | D-(+)- Biotin | 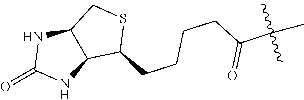 [SMILES: *C(=O)CCCC[C@@H]1SC[C@@H]2NC(=O)N[C@@H]21] | 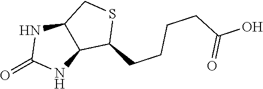 [SMILES: O=C(O)CCCC[C@@H]1SC[C@@H]2NC(=O)N[C@@H]21] |
| Bta | 3-(3- benzo- thienyl)- L-alanine | 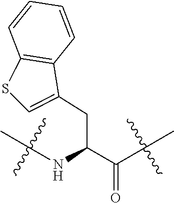 [SMILES: *N[C@@H](Cc1csc2ccccc12)C(*)=O] | 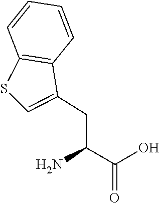 [SMILES: N[C@@H](Cc1csc2ccccc12)C(=O)O] |
| Bzl | Benzyl | 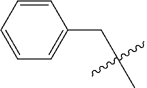 [SMILES: *Cc1ccccc1] | |
| Cha | L-cyclo- hexyl- alanine | 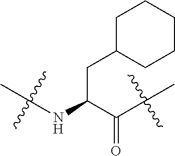 [SMILES: *N[C@@H](CC1CCCCC1)C(*)=O] | 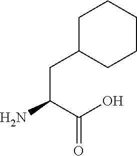 [SMILES: N[C@@H](CC1CCCCC1)C(=O)O] |
| Chg | L-cyclo- hexyl- glycine | 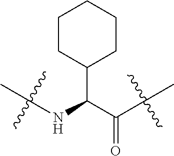 [SMILES: *N[C@H](C(*)=O)C1CCCCC1] | 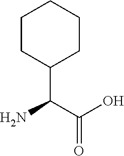 [SMILES: N[C@H](C(=O)O)C1CCCCC1] |
| Chy | 4-(S)- hydroxy-L- proline | 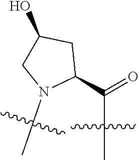 [SMILES: *C(=O)[C@@H]1C[C@H](O)CN1*] | 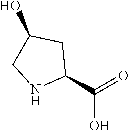 |
| Cfp | (2S,4S)- 4-fluoro- pyrrolidine- 2- carboxylic acid | 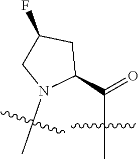 [SMILES: *C(=O)[C@@H]1C[C@H](F)CN1*] | 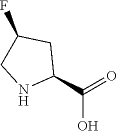 |
| Cit | L-Citrulline | 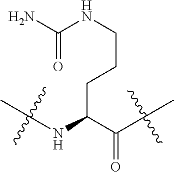 [SMILES: *N[C@@H](CCCNC(N)=O)C(*)=O] | 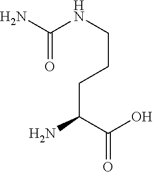 [SMILES: NC(=O)NCCC[C@H](N)C(=O)O] |
| Cpp | trans-3- Azabicyclo [3.1.0] hexane-2- carboxylic acid | 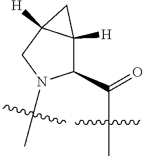 [SMILES: *C(=O)[C@@H]1[C@H]2C[C@H]2CN1*] | 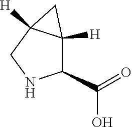 |
| Cy5SO3 | Cy5 dye (mono SO3) | 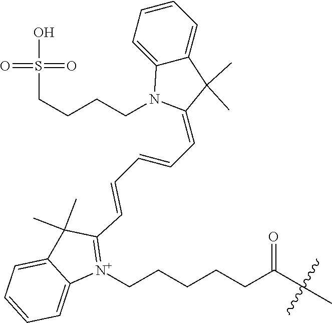 [SMILES: *C(=O)CCCCC[N+]1=C(/C=C/C=C/C=C2\N(CCCCS(=O)(=O)O)c3ccccc3C2(C)C)C(C)(C)c2ccccc21] | 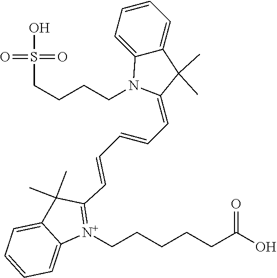 [SMILES: CC1(C)C(/C=C/C=C/C=C2\N(CCCCS(=O)(=O)O)c3ccccc3C2(C)C)=[N+](CCCCCC(=O)O)c2ccccc21] |
| Cya | L-cysteic acid | 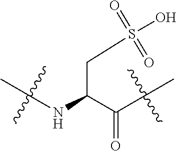 [SMILES: *N[C@@H](CS(=O)(=O)O)C(*)=O] | 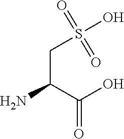 |
| cys | D- cysteine | 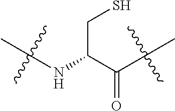 [SMILES: *N[C@H](CS)C(*)=O] | 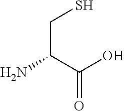 |
| Cys | L- cysteine | 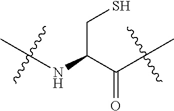 [SMILES: *N[C@@H](CS)C(*)=O] | 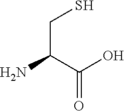 |
| Cys (2Lut) | L-cysteine bound to 2,6- lutidylidene | 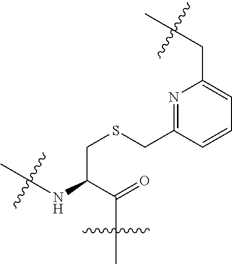 [SMILES: *Cc1cccc(CSC[C@H](N*)C(*)=O)n1] | |
| Cys (3Lut) | L-cysteine bound to 3,5- lutidylidene | 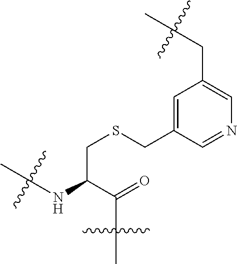 [SMILES: *Cc1cncc(CSC[C@H](N*)C(*)=O)c1] | |
| Cys (3MeBn) | L-cysteine bound to 3- methyl- benzylidene | 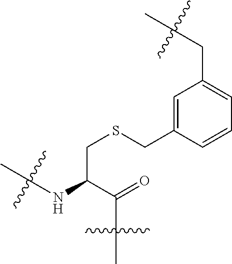 [SMILES: *Cc1cccc(CSC[C@H](N*)C(*)=O)c1] | |
| Cys(mli) | L-cysteine bound to maleimide | 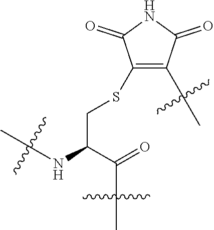 [SMILES: *N[C@@H](CSC1=C(*)C(=O)NC1=O)C(*)=O] | |
| Cys (tMeBn (DOTA- AET)) | L-cysteine conjugated to mesitylene conjugated to DOTA via 2- aminoethane- 1-thiol | 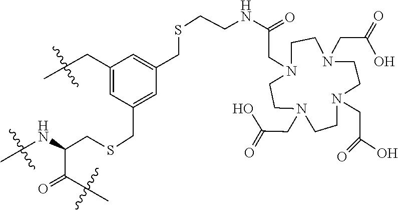 [SMILES: *Cc1cc(CSCCNC(=O)CN2CCN(CC(=O)O)CCN(CC(=O)O)CCN(CC(=O)O)CC2)cc(CSC[C@H](N*)C(*)=O)c1] | |
| Cys (tMeBn (H-AET)) | L-cysteine conjugates to mesitylene conjugates with 2- amino- ethane-1- thiol linker | 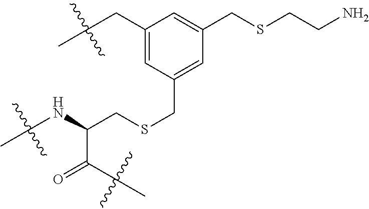 [SMILES: *Cc1cc(CSCCN)cc(CSC[C@H](N*)C(*)=O)c1] | |
| Cys (tMeBn (DOTA- PP)) | L-cysteine conjugated to mesitylene conjugated to DOTA via 2- amino- ethane-1- thiol | 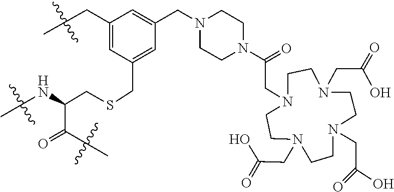 [SMILES: *Cc1cc(CSC[C@H](N*)C(*)=O)cc(CN2CCN(C(=O)CN3CCN(CC(=O)O)CCN(CC(=O)O)CCN(CC(=O)O)CC3)CC2)c1] | |
| Cys (tMeBn (H-PP)) | L-cysteine conjugated to mesitylene conjugated with piperazine linker | 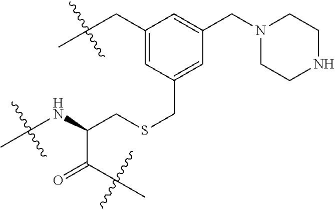 [SMILES: *Cc1cc(CSC[C@H](N*)C(*)=O)cc(CN2CCNCC2)c1] | |
| Cysol | L- cysteinol | 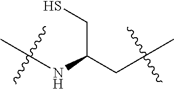 [SMILES: *C[C@H](CS)N*] | 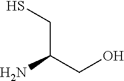 |
| Dab | (S)-2,4- Diamino- butyric acid | 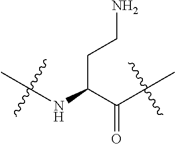 [SMILES: *N[C@@H](CCN)C(*)=O] | 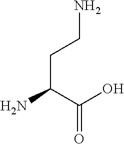 |
| Dap | (S)-2,3- Diamino- propionic acid | 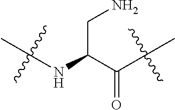 [SMILES: *N[C@@H](CN)C(*)=O] | 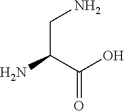 |
| dap | (R)-2,3- Diamino- propionic acid | 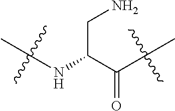 [SMILES: *N[C@H](CN)C(*)=O] | 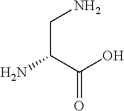 |
| Deg | Diethyl- glycine | 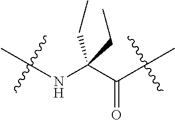 | 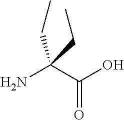 |
| Dfp | 4,4- Difluoro-L- proline | 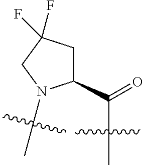 [SMILES: *C(=O)[C@@H]1CC(F)(F)CN1*] | 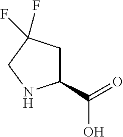 |
| Dmp | (S)-5,5- Dimethyl- proline | 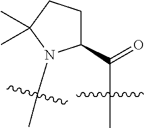 [SMILES: *C(=O)[C@@H]1CCC(C)(C)N1*] | 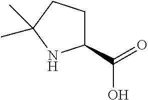 |
| DOTA | 2,2′,2″,2′′′- (1,4,7,10- Tetraaza- cyclo- dodecane- 1,4,7,10- tetrayl) tetraacetic acid | 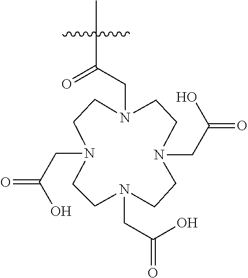 [SMILES: *C(=O)CN1CCN(CC(=O)O)CCN(CC(=O)O)CCN(CC(=O)O)CC1] | 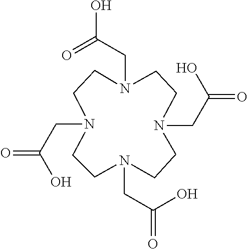 [SMILES: O=C(O)CN1CCN(CC(=O)O)CCN(CC(=O)O)CCN(CC(=O)O)CC1] |
| Dtc | (R)-5,5- dimethyl- 1,3- thiazolidine- 4- carboxylic acid | 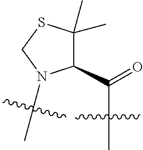 [SMILES: *C(=O)[C@H]1N(*)CSC1(C)C] | 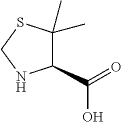 |
| Eaa | 3,4- Dichlor-L- phenyl- alanine | 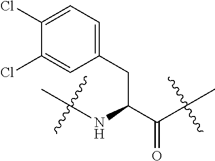 [SMILES: *N[C@@H](Cc1ccc(Cl)c(Cl)c1)C(*)=O] | 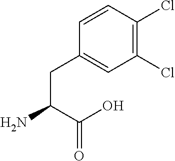 [SMILES: N[C@@H](Cc1ccc(Cl)c(Cl)c1)C(=O)O] |
| Eaz | L- thiazolidine- 4- carboxylic acid | 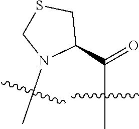 [SMILES: *C(=O)[C@@H]1CSCN1*] | 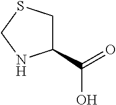 |
| Eca | 1-amino-1- cyclo- pentane carboxylic acid | 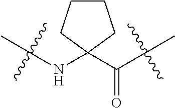 [SMILES: *NC1(C(*)=O)CCCC1] | 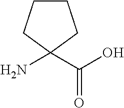 |
| Egd | (S)-ω,ω- Dimethyl- arginine | 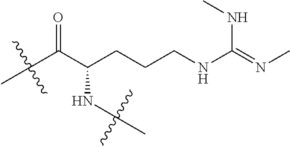 [SMILES: *N[C@@H](CCCN/C(=N/C)NC)C(*)=O] | 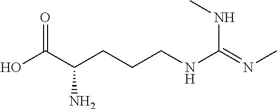 |
| Egm | 2,4- dichloro-L- phenyl- alanine | 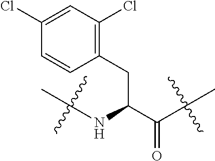 [SMILES: *N[C@@H](Cc1ccc(Cl)cc1Cl)C(*)=O] | 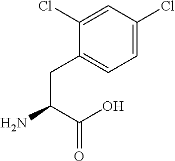 [SMILES: N[C@@H](Cc1ccc(Cl)cc1Cl)C(=O)O] |
| Egn | 3,5- dichloro-L- phenyl- alanine | 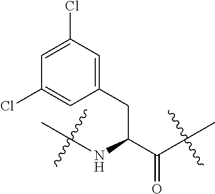 [SMILES: *N[C@@H](Cc1cc(Cl)cc(Cl)c1)C(*)=O] | 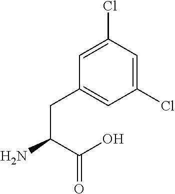 [SMILES: N[C@@H](Cc1cc(Cl)cc(Cl)c1)C(=O)O] |
| Egp | 2,3- dichloro-L- phenyl- alanine | 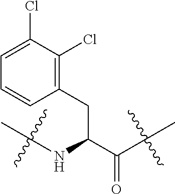 [SMILES: *N[C@@H](Cc1cccc(Cl)c1Cl)C(*)=O] | 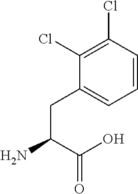 [SMILES: N[C@@H](Cc1cccc(Cl)c1Cl)C(=O)O] |
| Egz | 1-Amino-1- cyclohexan- carboxylic acid | 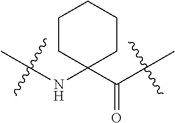 [SMILES: *NC1(C(*)=O)CCCCC1] | 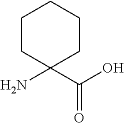 |
| en | Ethylen- diamine | 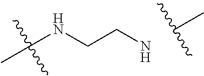 | 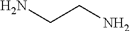 |
| ‘en’ | Ethylen- diamine | 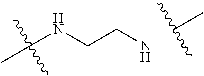 | 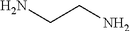 |
| EuDOTA | DOTA Europium (III) complex | 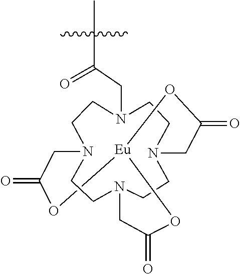 | |
| GaDOTA | DOTA Gallium(III) complex | 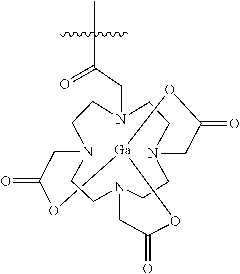 | |
| GaNO- DAGA | NODAGA Gallium(III) complex | 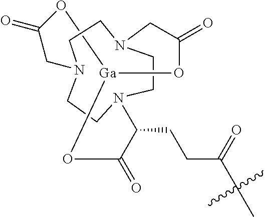 | |
| Ghg | γ-Hydroxy- L- glutamic acid | 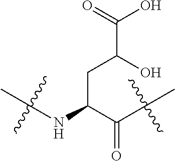 [SMILES: *N[C@@H](CC(O)C(=O)O)C(*)=O] | 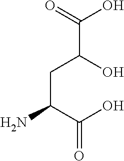 [SMILES: N[C@@H](CC(O)C(=O)O)C(=O)O] |
| gln | D- glutamine | 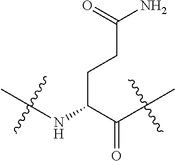 [SMILES: *N[C@H](CCC(N)=O)C(*)=O] | 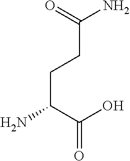 |
| Gln | L- glutamine | 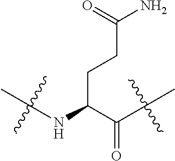 [SMILES: *N[C@@H](CCC(N)=O)C(*)=O] | 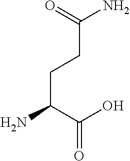 |
| glu | D-glutamic aicd | 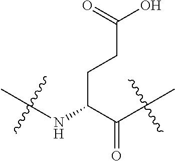 [SMILES: *N[C@H](CCC(=O)O)C(*)=O] | 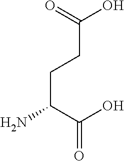 |
| Glu | L-glutamic aicd | 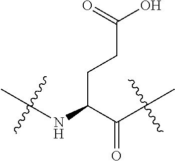 [SMILES: *N[C@@H](CCC(=O)O)C(*)=O] | 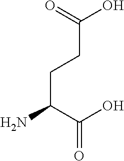 |
| GLU- SEROL | | 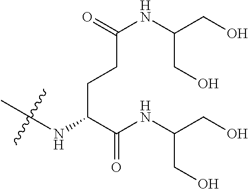 [SMILES: *N[C@H](CCC(=O)NC(CO)CO)C(=O)NC(CO)CO] | 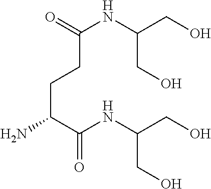 [SMILES: N[C@H](CCC(=O)NC(CO)CO)C(=O)NC(CO)CO] |
| glu (AGLU) | glu(AGLU) conjugate | 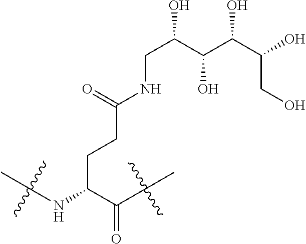 [SMILES: *N[C@H](CCC(=O)NC[C@H](O)[C@@H](O)[C@H](O)[C@H](O)CO)C(*)=O] | 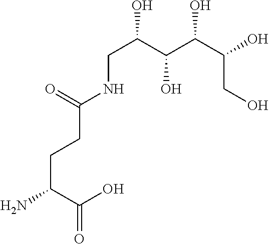 [SMILES: N[C@H](CCC(=O)NC[C@H](O)[C@@H](O)[C@H](O)[C@H](O)CO)C(=O)O] |
| Glutar | glutaric acid as linker | 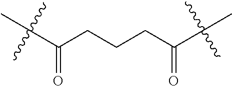 [SMILES: *C(=O)CCCC(*)=O] | 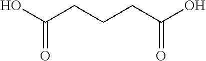 |
| Glutar | glutaric acid when present in the position of Xaa1 | 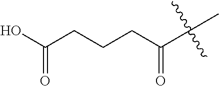 [SMILES: *C(=O)CCCC(=O)O] | 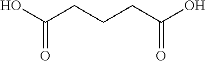 |
| Gly | glycine | 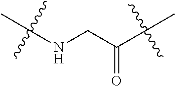 [SMILES: *NCC(*)=O] | 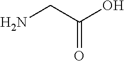 |
| H | Hydrogen- usually represents a free | 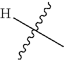 [SMILES: *[H]] | |
| | peptide | | |
| | N-terminus | | |
| H3p | 3-(S)- hydroxy-L- proline | 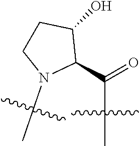 [SMILES: *C(=O)[C@@H]1[C@@H](O)CCN1*] | 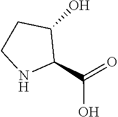 |
| Har | L-Homo- arginine | 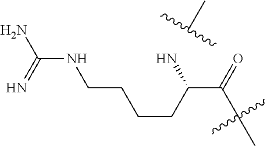 | 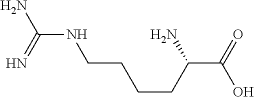 [SMILES: N=C(N)NCCCC[C@H](N)C(=O)O] |
| Hci | L-Homo- citrulline | 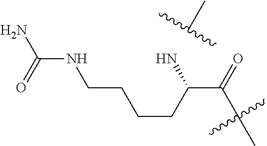 | 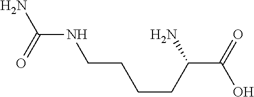 [SMILES: NC(=O)NCCCC[C@H](N)C(=O)O] |
| Hcy | L-homo- cysteine | 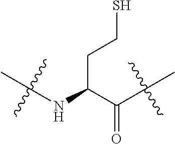 [SMILES: *N[C@@H](CCS)C(*)=O] | 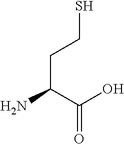 |
| Hga | L-homo- glutamic acid | 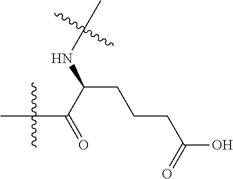 [SMILES: *N[C@@H](CCCC(=O)O)C(*)=O] | 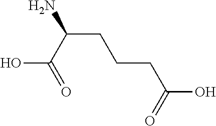 [SMILES: N[C@@H](CCCC(=O)O)C(=O)O] |
| Hfe | L- homophenyl- alanine | 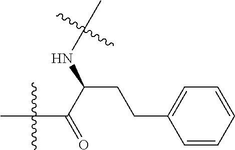 [SMILES: *N[C@@H](CCc1ccccc1)C(*)=O] | 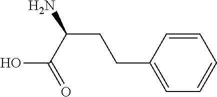 [SMILES: N[C@@H](CCc1ccccc1)C(=O)O] |
| Hle | L-homo- leucine | 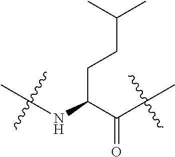 [SMILES: *N[C@@H](CCC(C)C)C(*)=O] | 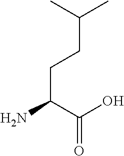 |
| Hse | L-homo- serine | 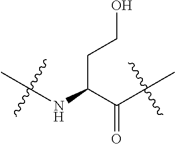 [SMILES: *N[C@@H](CCO)C(*)=O] | 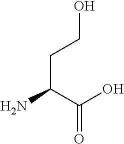 |
| Hth | L-homo- threonine | 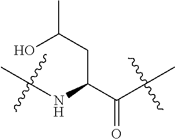 [SMILES: *N[C@@H](CC(C)O)C(*)=O] | 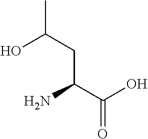 |
| Hym | (2S,4R)-4- Methyoxy- pyrrolidine- 2- carboxylic acid | 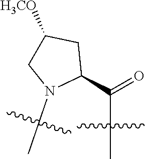 [SMILES: *C(=O)[C@@H]1C[C@@H](OC)CN1*] | 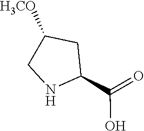 |
| Hyp | 4-(R)- hydroxy-L- proline | 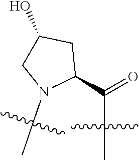 [SMILES: *C(=O)[C@@H]1C[C@@H](O)CN1*] | 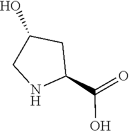 |
| Hyw | 5- hydroxy-L- tryptophane | 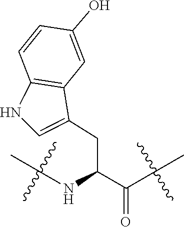 [SMILES: *N[C@@H](Cc1c[nH]c2ccc(O)cc12)C(*)=O] | 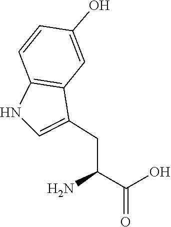 [SMILES: N[C@@H](Cc1c[nH]c2ccc(O)cc12)C(=O)O] |
| Ile | L- isoleucine | 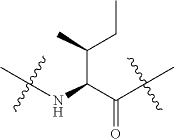 [SMILES: *N[C@H](C(*)=O)[C@@H](C)CC] | 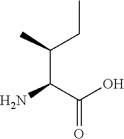 |
| InDOTA | DOTA Indium(III) complex | 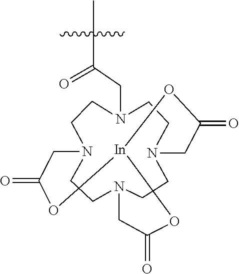 | |
| leu | D-leucine | 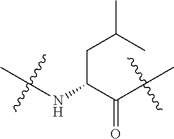 [SMILES: *N[C@H](CC(C)C)C(*)=O] | 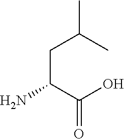 |
| Leu | L-leucine | 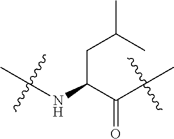 [SMILES: *N[C@@H](CC(C)C)C(*)=O] | 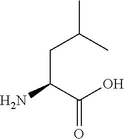 |
| LuDOTA | DOTA Lutetium (III) complex | 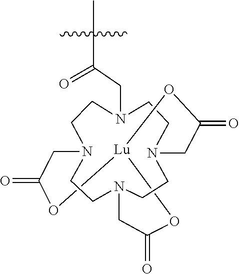 | |
| lys | D-lysine | 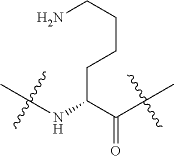 [SMILES: *N[C@H](CCCCN)C(*)=O] | 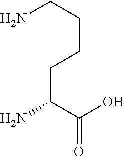 |
| Lys | L-lysine | 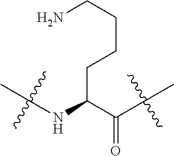 [SMILES: *N[C@@H](CCCCN)C(*)=O] | 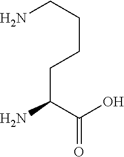 |
| Mcf | 3-chloro-L- phenyl- alanine | 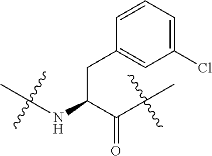 [SMILES: *N[C@@H](Cc1cccc(Cl)c1)C(*)=O] | 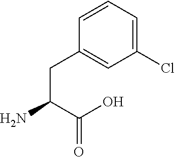 [SMILES: N[C@@H](Cc1cccc(Cl)c1)C(=O)O] |
| MeO10k- DaPE GAc | Polydisperse 2 kDa PEG | 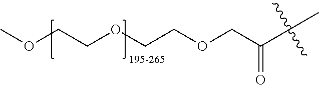 | |
| MeO2k- DaPE GAc | Polydisperse 10 kDa PEG | 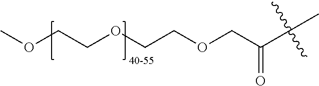 | |
| MeO36- PEG | Mono- disperse polyethylene glycol 36 units conjugated with acetyl | 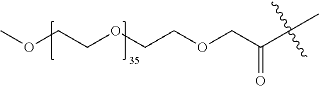 | |
| Met | L- methionine | 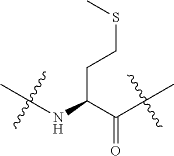 [SMILES: *N[C@@H](CCSC)C(*)=O] | 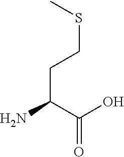 |
| mli | maleimide | 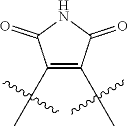 [SMILES: *C1=C(*)C(=O)NC1=O] | |
| Moo | (S)- Methionine sulfone | 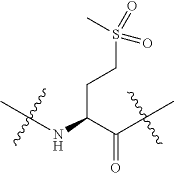 [SMILES: *N[C@@H](CCS(C)(=O)=O)C(*)=O] | 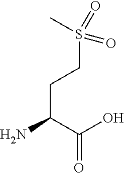 |
| N3Tap | (2S,4R)- 4-azido-1- pyrrolidin- 2- carboxylic acid | 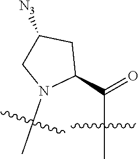 [SMILES: *C(=O)[C@@H]1C[C@@H](N=[N+]=[N-])CN1*] | 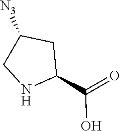 [SMILES: [N-]=[N+]=N[C@H]1CN[C@H](C(=O)O)C1] |
| N4Ac | Bis-(2- amino- ethylamine)- methyl- propionic acid | 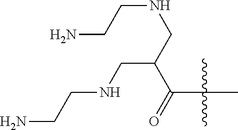 [SMILES: *C(=O)C(CNCCN)CNCCN] | 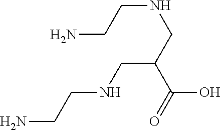 [SMILES: NCCNCC(CNCCN)C(=O)O] |
| Nglu | N-glutamic acid, N-carboxy- ethyl glycine | 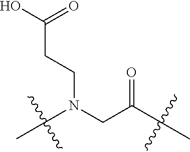 [SMILES: *C(=O)CN(*)CCC(=O)O] | 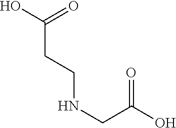 |
| NH₂ | usually represents a C-terminus of a | 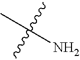 [SMILES: *N] | |
| | peptide | | |
| | sequence | | |
| Nle | L- norleucine | 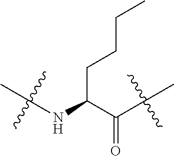 [SMILES: *N[C@@H](CCCC)C(*)=O] | 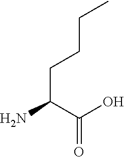 |
| Nleu | N-leucine, N-isobutyl glycine | 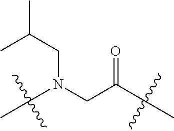 | 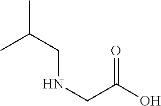 |
| Nlys | N-lysine, N-(4- aminobutyl) glycine | 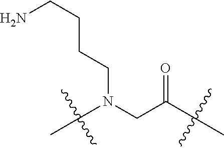 [SMILES: *C(=O)CN(*)CCCCN] | 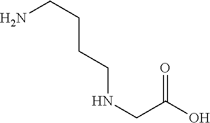 |
| nma | N-methyl- D-alanine | 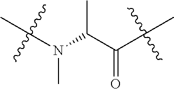 | 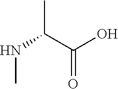 |
| Nma | N-methyl- L-alanine | 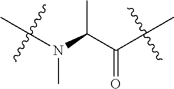 [SMILES: *C(=O)[C@H](C)N(*)C] | 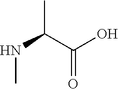 |
| NMc | N-methyl- L- cysteine | 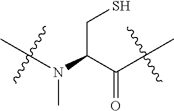 | 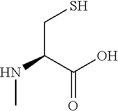 |
| Nmd | N-methyl- L- aspartic acid | 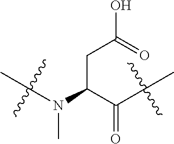 [SMILES: *C(=O)[C@H](CC(=O)O)N(*)C] | 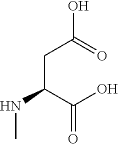 |
| Nme | N-methyl- L- glutamic acid | 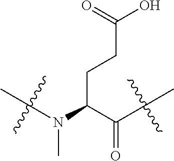 [SMILES: *C(=O)[C@H](CCC(=O)O)N(*)C] | 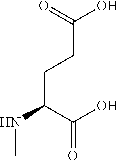 |
| Nmg | N-methyl- glycine | 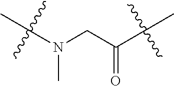 [SMILES: *C(=O)CN(*)C] | 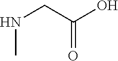 |
| Nml | N-methyl- L-leucine | 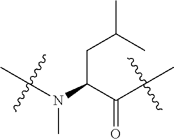 [SMILES: *C(=O)[C@H](CC(C)C)N(*)C] | 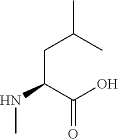 |
| Nmw | N-methyl- L- tryptophan | 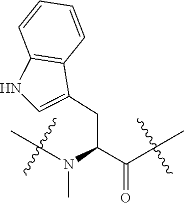 [SMILES: *C(=O)[C@H](Cc1c[nH]c2ccccc12)N(*)C] | 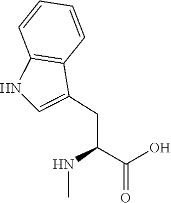 [SMILES: CN[C@@H](Cc1c[nH]c2ccccc12)C(=O)O] |
| NODAGA | (R)-1,4,7- triaza- cyclononane, 1-glutaric acid-4,7- acetic acid | 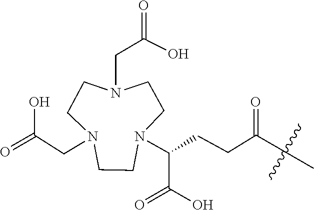 [SMILES: *C(=O)CC[C@H](C(=O)O)N1CCN(CC(=O)O)CCN(CC(=O)O)CC1] | 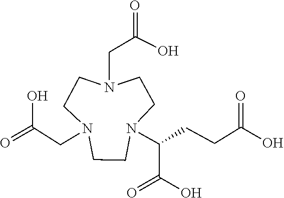 [SMILES: O=C(O)CC[C@H](C(=O)O)N1CCN(CC(=O)O)CCN(CC(=O)O)CC1] |
| NOPO | 1,4,7- triazacyclo- nonane- 1,4-bis [methylene (hydroxy- methyl) phosphinic acid]-7- [methylene (2- carboxyethyl) | 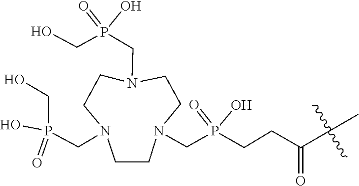 [SMILES: *C(=O)CCP(=O)(O)CN1CCN(CP(=O)(O)CO)CCN(CP(=O)(O)CO)CC1] | 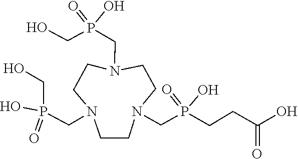 [SMILES: O=C(O)CCP(=O)(O)CN1CCN(CP(=O)(O)CO)CCN(CP(=O)(O)CO)CC1] |
| | phosphinic | | |
| | acid] | | |
| Npg | L-neopentyl- glycine | 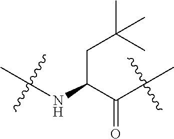 [SMILES: *N[C@@H](CC(C)(C)C)C(*)=O] | 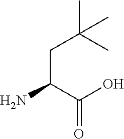 |
| Nph1p | 3- Naphthalen- 1-yl- propionic acid | 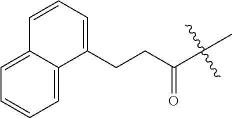 [SMILES: *C(=O)CCc1cccc2ccccc12] | 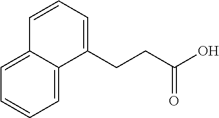 [SMILES: O=C(O)CCc1cccc2ccccc12] |
| Nph2p | 3- Naphthalen- 2-yl- propionic acid | 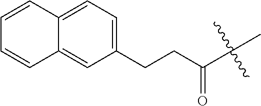 [SMILES: *C(=O)CCc1ccc2ccccc2c1] | 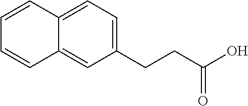 [SMILES: O=C(O)CCc1ccc2ccccc2c1] |
| Nphe | N-phenyl- alanine, N-benzyl- glycine | 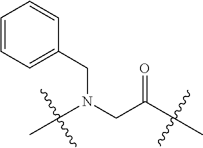 [SMILES: *C(=O)CN(*)Cc1ccccc1] | 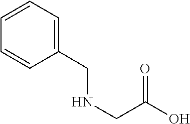 [SMILES: O=C(O)CNCc1ccccc1] |
| Nta | nitrilo- triacetic acid | 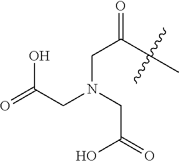 [SMILES: *C(=O)CN(CC(=O)O)CC(=O)O] | 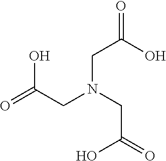 [SMILES: O=C(O)CN(CC(=O)O)CC(=O)O] |
| NTA- SEROL | | 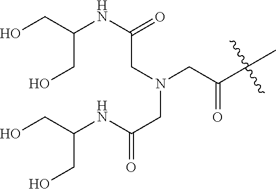 [SMILES: *C(=O)CN(CC(=O)NC(CO)CO)CC(=O)NC(CO)CO] | 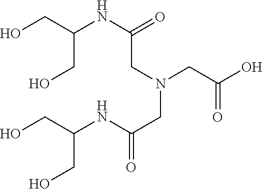 [SMILES: O=C(O)CN(CC(=O)NC(CO)CO)CC(=O)NC(CO)CO] |
| Nva | L- norvaline | 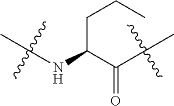 [SMILES: *N[C@@H](CCC)C(*)=O] | 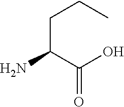 |
| O2Oc | 8-amino- 3,6- dioxaoctanoic acid | 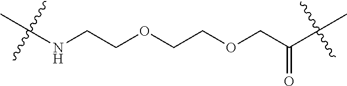 [SMILES: *NCCOCCOCC(*)=O] | 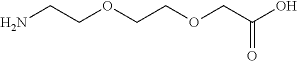 [SMILES: NCCOCCOCC(=O)O] |
| Ocf | ortho- chloro-L- phenyl- alanine | 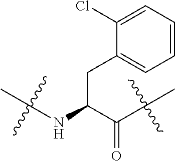 [SMILES: *N[C@@H](Cc1ccccc1Cl)C(*)=O] | 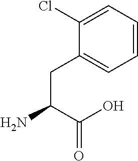 [SMILES: N[C@@H](Cc1ccccc1Cl)C(=O)O] |
| OH | usually represents a C-terminus | 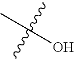 [SMILES: *O] | |
| | of a | | |
| | peptide | | |
| | sequence | | |
| Oic | (2S,3aS, 7aS)- octahydro- indole- carboxylic acid | 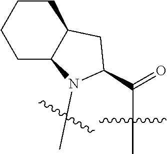 [SMILES: *C(=O)[C@@H]1C[C@@H]2CCCC[C@@H]2N1*] | 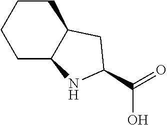 [SMILES: O=C(O)[C@@H]1C[C@@H]2CCCC[C@@H]2N1] |
| Omr | ω- Methyl-L- arginine | 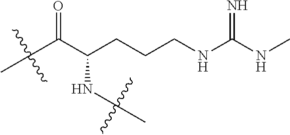 [SMILES: *N[C@@H](CCCNC(=N)NC)C(*)=O] | 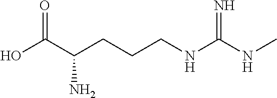 |
| Oxa | (S)-oxa- zolidine-4- carboxylic acid | 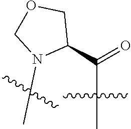 [SMILES: *C(=O)[C@@H]1COCN1*] | 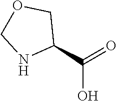 |
| Pcf | para- chloro-L- phenyl- alanine | 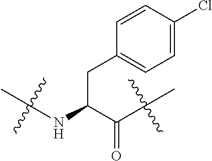 [SMILES: *N[C@@H](Cc1ccc(Cl)cc1)C(*)=O] | 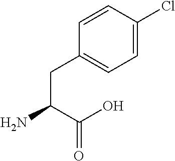 [SMILES: N[C@@H](Cc1ccc(Cl)cc1)C(=O)O] |
| PEG6 | 21-amino- 4,7,10,13, 16,19- hexa- oxahenei- cosanoic acid | 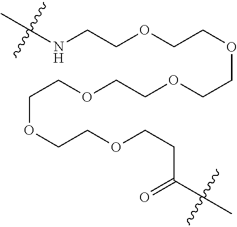 [SMILES: *NCCOCCOCCOCCOCCOCCOCCC(*)=O] | 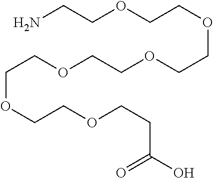 [SMILES: NCCOCCOCCOCCOCCOCCOCCC(=O)O] |
| Pen | L- penicillamine | 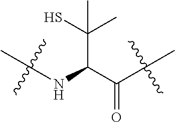 [SMILES: *N[C@H](C(*)=O)C(C)(C)S] | 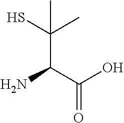 |
| Phe | L- phenylalanine | 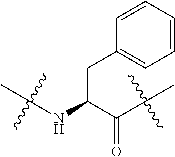 [SMILES: *N[C@@H](Cc1ccccc1)C(*)=O] | 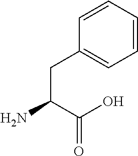 [SMILES: N[C@@H](Cc1ccccc1)C(=O)O] |
| Phg | L- phenylglycine | 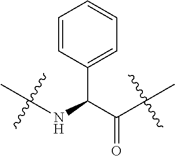 [SMILES: *N[C@H](C(*)=O)c1ccccc1] | 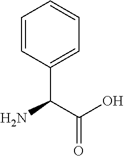 [SMILES: N[C@H](C(=O)O)c1ccccc1] |
| Php | 3-phenyl- propionic acid | 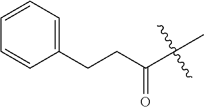 [SMILES: *C(=O)CCc1ccccc1] | 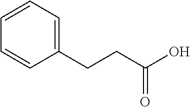 [SMILES: O=C(O)CCc1ccccc1] |
| Pip | (S)- Pipecolic Acid | 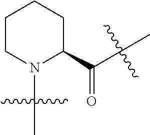 [SMILES: *C(=O)[C@@H]1CCCCN1*] | 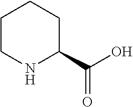 |
| PP | Piperazinyl/ Piperazine | 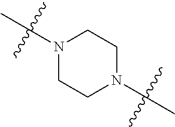 | 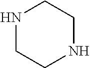 |
| pro | D-proline | 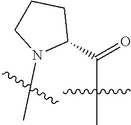 [SMILES: *C(=O)[C@H]1CCCN1*] | 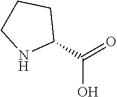 |
| Pro | L-proline | 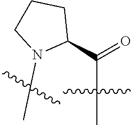 [SMILES: *C(=O)[C@@H]1CCCN1*] | 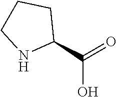 |
| Pse | L- phosphoserine | 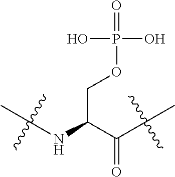 [SMILES: *N[C@@H](COP(=O)(O)O)C(*)=O] | 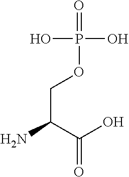 [SMILES: N[C@@H](COP(=O)(O)O)C(=O)O] |
| Rni | (R)- nipecotic acid | 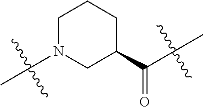 | 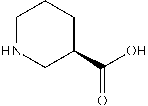 |
| Ser | L-serine | 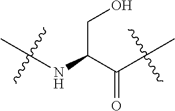 [SMILES: *N[C@@H](CO)C(*)=O] | 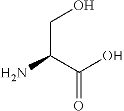 |
| Serol | serinol | 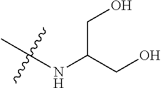 [SMILES: *NC(CO)CO] | 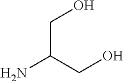 |
| Smc | S-methyl-L- cysteine | 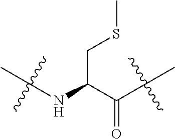 [SMILES: *N[C@@H](CSC)C(*)=O] | 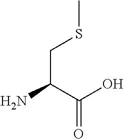 |
| Succinyl | succinic acid as linker | 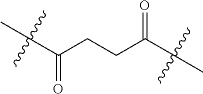 [SMILES: *C(=O)CCC(*)=O] | 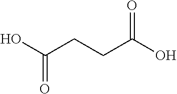 |
| Succinyl | succinic acid when present in the position of Xaa1 | 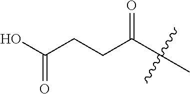 [SMILES: *C(=O)CCC(=O)O] | 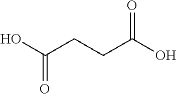 |
| Tap | (2S,4R)- 4-amino-1- pyrrolidin- 2- carboxylic acid | 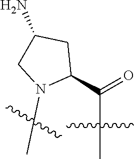 [SMILES: *C(=O)[C@@H]1C[C@@H](N)CN1*] | 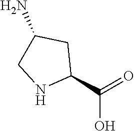 |
| Tap (AGLU- Glutar) | (2S,4R)- 4-amino-1- pyrrolidin-2- carboxylic acid (AGLU- Glutar) conjugate | 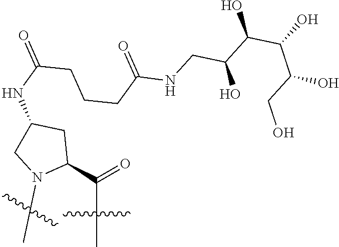 [SMILES: *C(=O)[C@@H]1C[C@@H](NC(=O)CCCC(=O)NC[C@H](O)[C@@H](O)[C@H](O)[C@H](O)CO)CN1*] | |
| Tfp | (2S,4R)- 4-fluoro- pyrrolidine- 2- carboxylic acid | 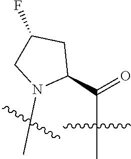 [SMILES: *C(=O)[C@@H]1C[C@@H](F)CN1*] | 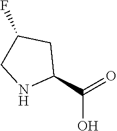 |
| Thi | L- thienylalanine | 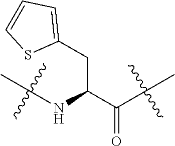 [SMILES: *N[C@@H](Cc1cccs1)C(*)=O] | 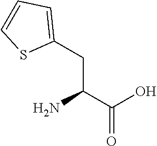 [SMILES: N[C@@H](Cc1cccs1)C(=O)O] |
| Thp | 4-amino- tetrahydro- pyran-4- carboxylic acid | 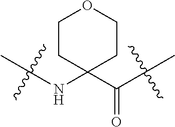 [SMILES: *NC1(C(*)=O)CCOCC1] | 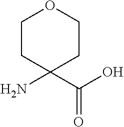 |
| Thr | L-threonine | 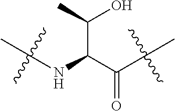 [SMILES: *N[C@H](C(*)=O)[C@@H](C)O] | 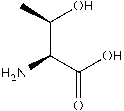 |
| Tle | (S)-α-t- butylglycine | 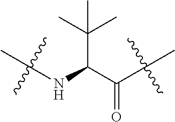 [SMILES: *N[C@H](C(*)=O)C(C)(C)C] | 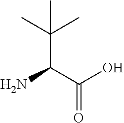 |
| tMeBn | tris- methylbenzyl, mesitylene | 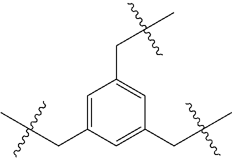 [SMILES: *Cc1cc(C*)cc(C*)c1] | |
| tMeBn (DOTA- AET) | DOTA attached to mesitylene via 2- aminoethane- 1- thiol | 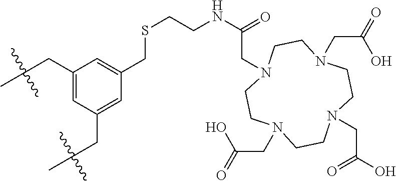 [SMILES: *Cc1cc(C*)cc(CSCCNC(=O)CN2CCN(CC(=O)O)CCN(CC(=O)O)CCN(CC(=O)O)CC2)c1] | |
| tMeBn (DOTA- PP) | DOTA attached to mesitylene via piperazine | 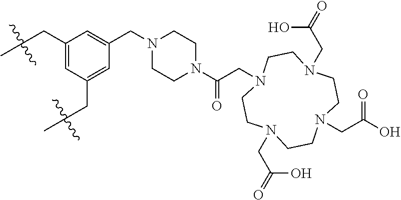 [SMILES: *Cc1cc(C*)cc(CN2CCN(C(=O)CN3CCN(CC(=O)O)CCN(CC(=O)O)CCN(CC(=O)O)CC3)CC2)c1] | |
| trp | D- tryptophan | 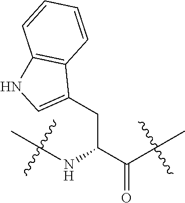 [SMILES: *N[C@H](Cc1c[nH]c2ccccc12)C(*)=O] | 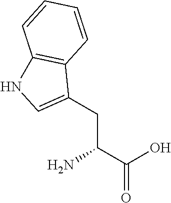 [SMILES: N[C@H](Cc1c[nH]c2ccccc12)C(=O)O] |
| Trp | L- tryptophan | 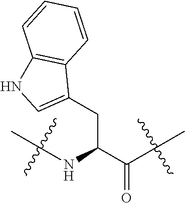 [SMILES: *N[C@@H](Cc1c[nH]c2ccccc12)C(*)=O] | 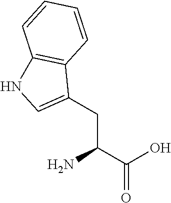 [SMILES: N[C@@H](Cc1c[nH]c2ccccc12)C(=O)O] |
| Ttds | 1,13- diamino- 4,7,10- tioxa- tridecan- succinamic acid | 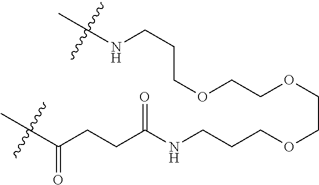 [SMILES: *NCCCOCCOCCOCCCNC(=O)CCC(*)=O] | 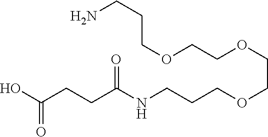 [SMILES: NCCCOCCOCCOCCCNC(=O)CCC(=O)O] |
| tyr | D- tyrosine | 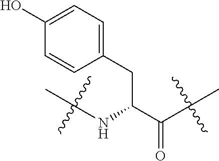 [SMILES: *N[C@H](Cc1ccc(O)cc1)C(*)=O] | 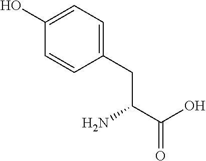 [SMILES: N[C@H](Cc1ccc(O)cc1)C(=O)O] |
| Tyr | L- tyrosine | 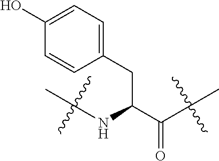 [SMILES: *N[C@@H](Cc1ccc(O)cc1)C(*)=O] | 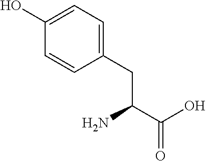 [SMILES: N[C@@H](Cc1ccc(O)cc1)C(=O)O] |
| val | D-valine | 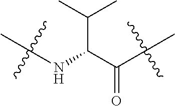 [SMILES: *N[C@@H](C(*)=O)C(C)C] | 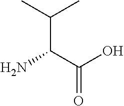 |
| Val | L-valine | 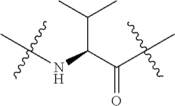 [SMILES: *N[C@H](C(*)=O)C(C)C] | 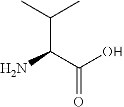 |
| ZnDOTA | DOTA Zink (II) complex | 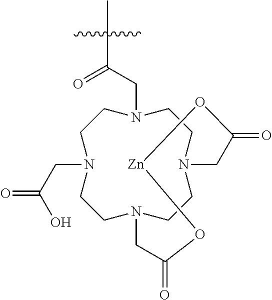 | |

The amino acid sequences of the peptides disclosed herein are depicted in typical peptide sequence format, as will be understood by a person skilled in the art. For example, the three-letter code for a residue of a conventional amino acid, the code for a residue of a non-conventional amino acid or the abbreviation for a residue of a building block other than a conventional or non-conventional amino acid indicates the presence of the residue, of the amino acid residue or the presence of the residue of the building block at a specified position within the peptide sequence. The code for a residue of an amino acid and the abbreviation for a residue of a building block, respectively, is connected to the code for a subsequent residue of an amino acid and/or to the code for a preceding residue of an amino acid and, respectively, is connected to the abbreviation for a subsequent residue of a building block and/or to the abbreviation for a preceding residue of a building block, respectively, in the peptide sequence by a hyphen which typically represents an amide linkage. For example, if the hyphen is located before the code for a residue of an amino acid, this usually symbolizes that the amino group (as indicated in the corresponding entry in table 5) of the residue of the amino acid is covalently attached to a carbonyl group of a preceding residue of an amino acid, and if the hyphen is located after the code for a residue of an amino acid, this usually symbolizes that the carbonyl group (as indicated in the corresponding entry in table 5) of the residue of the amino acid is covalently attached to an amino group of a subsequent residue of an amino acid.

Depending on the spacing between the amino- and the carboxy group in amino acids they are classified into α-, β-, γ-, δ-, ε-, (and so forth) -amino acids, which means that these groups are typically are spaded apart by 1, 2, 3, 4, and 5 heavy-atoms (typically carbon), respectively.

For amino acids, in their abbreviations the first letter indicates the stereochemistry of the C-α-atom if applicable. For example, a capital first letter indicates that the L-form of the amino acid is present in the peptide sequence, while a lower case first letter indicating that the D-form of the correspondent amino acid is present in the peptide sequence. If the abbreviation starts with a number, the first letter in the abbreviation will be characteristic for the stereochemistry, if applicable. However, for enhanced clarity it is at any abbreviation an option to further clearly specify the stereochemistry of an amino acid abbreviation by adding for instance the prefix “D-”. As example “lys”, “D-Lys” or “D-lys” describe all a D-configured Lys.

For someone skilled in the art it is evident that many amino acids can be N-methylated at their amino group. These N-methyl amino acid feature can occur in combination with some other attributes like L-α- or D-α-N-methyl amino acids which are N-methylated L-α- or D-α-amino acids.

The term “α,α-dialkylamino acid” refers to amino acid which comprise independently two alkyl groups at the α-carbon atom which maybe in some cases form a ring-structure with each other.

The term “cyclic” amino acid means that the amino acid comprises a cyclic structure wherein in some cases the amino nitrogen of the amino acid is part of a heterocyclic structure usually consisting of 4 to 7 ring members. Cyclic maybe combined with other amino acid attribute as for instance in cyclic D-α-amino acid or cyclic α,α-dialkylamino acid.

The term “aromatic amino acid” refers to amino acids which comprise an aromatic structure and this includes a heteroaromatic structure whereas the term “non-aromatic amino acid” refers to amino acids which are devoid of any aromatic structure.

The term “heteroaromatic amino acid” refers to amino acids which comprise any kind of heteroaromatic structure.

An “aliphatic amino acid” is a non-aromatic amino acid which consists of only C and H atoms apart from the amino and carboxy group.

A “polar amino acid” is any kind of amino acid which comprises apart from the amino and carboxy group functional groups or atoms selected from the group consisting of O, N, S, P, CONH₂, COOH, CN and tetrazole.

A “charged amino acid” is any kind of amino acid which comprises, apart from the amino and carboxy group, functional groups which can be charged at a certain pH, esp. in the range of 4-8, preferably selected from the group of COOH, phosphate, phosphonate, sulfonate, ammonium and amino nitrogen.

A neutral amino acid is any kind of amino acid which comprises apart from the amino and carboxy group, no further functional groups which can be charged at a certain pH, esp. in the range of 4-8.

If an amino acid contains more than one amino and/or carboxy group all orientations of this amino acid are in principle possible for formation of a covalent bond, but in α-amino acid the utilization of the α-amino and the α-carboxy group is preferred for the attachment to the neighbouring moieties and if other orientations are preferred they are explicitly specified.

Those skilled in the art will recognize if a stereocenter exists in the compounds disclosed herein irrespective thereof whether such stereocenter is part of an amino acid moiety or any other part or moiety of the compound of the invention. When a compound is desired as a single enantiomer or diastereomer, it may be obtained by stereospecific synthesis or by resolution of the final product or any convenient intermediate. Resolution of the final product, an intermediate, or a starting material may be affected by any suitable method known in the art. See, for example, “Stereochemistry of Organic Compounds” by E. L. Eliel, S. H. Wilen, and L. N. Mander (Wiley-Interscience, 1994).

In an embodiment and as preferably used herein, an aromatic L-α-amino acid is any kind of L-α-amino acid which comprises an aryl group.

In an embodiment and as preferably used herein, a heteroaromatic L-α-amino acid is any kind of L-α-amino acid which comprises a heteroaryl group.

Unless indicated to the contrary, the amino acid sequences are presented herein in N- to C-terminus direction.

Derivatives of the amino acids constituting the peptides of the invention may be as set forth in Table 6. In any embodiment, one or more amino acids of the compounds of the invention are substituted with a derivative of the corresponding preferred amino acids.

TABLE 6
Exemplary derivatives of preferred amino acids
contained in the compound of the invention

| Amino | |
|---|---|
| Acid | Exemplary derivatives |
| Ala | Aib, Bal, Abu, Gly, Nva, Nle |
| Cys | Hcy, Nmc |
| Asp | Glu, Asn, Gln, Cya |
| Glu | Asp, Asn, Gln, Cya, Homoglutamic acid, y-Hydroxy- |
| | glutamic acid, |
| Phe | Hfe, Phg, Bhf, Thi, Bta, Bromophenylalanine, |
| | Iodophenylalanine, Chlorophenylalanine, |
| | Methylphenylalanine, Nitrophenylalanine, Tyr, Trp, |
| | Naphthylalanine, Trifluoromethylphenylalanine |
| Gly | Ala, ala, Nmg |
| Nmg | Pro, Ala, ala, Gly, Nma, nma |
| Ile | Leu, Val, Hle, Nva, Nle, Chg |
| Lys | Arg, Dab, Dap, Har, Egd, Omr, Hci, Cit |
| Leu | Ile, Val, Hle, Nle, Nva, Moo |
| Met | Ile, Val, Hle, Nle, Nva, Moo |
| Nle | Ile, Val, Hle, Met, Nva, Moo |
| Asn | Asp, Glu, Gln, Cya, Thr |
| Pro | Aze, Pip, Hyp, Tfp, Cfp, Dmp, Tap, H3p, 4Ap, Cpp, |
| | Hym, Chy, Dfp |
| Gln | Asp, Asn, Glu, Cya, Thr, Hse |
| Arg | Arg, Dab, Dap, Har, Egd, Omr, Hci, Cit |
| Ser | Thr, Hse, allo-Threonine |
| Thr | Ser, Homothreonine, allo-Threonine |
| Val | Leu, Ile, Hle, Nva, Nle |
| Trp | Hfe, Phg, Bhf, Thi, Bta, Bromophenylalanine, |
| | Iodophenylalanine, Chlorophenylalanine, |
| | Methylphenylalanine, Nitrophenylalanine, Tyr, Trp, |
| | Naphthylalanine, Trifluoromethylphenylalanine |
| Tyr | Hfe, Phg, Bhf, Thi, Bta, Bromophenylalanine, |
| | Iodophenylalanine, Chlorophenylalanine, |
| | Methylphenylalanine, Nitrophenylalanine, Tyr, Trp, |
| | Naphthylalanine, Trifluoromethylphenylalanine |

The following compounds are compounds of the present invention, but are not preferred compounds of the present invention:

compound H-Met-Tyr-Asp-Trp-Trp-Pro-Glu-Leu-[Cys(3MeBn)-Ala-Leu-Ile-Cys]-Ttds-Lys(Bio)-NH₂ (GIP-057) of the following formula

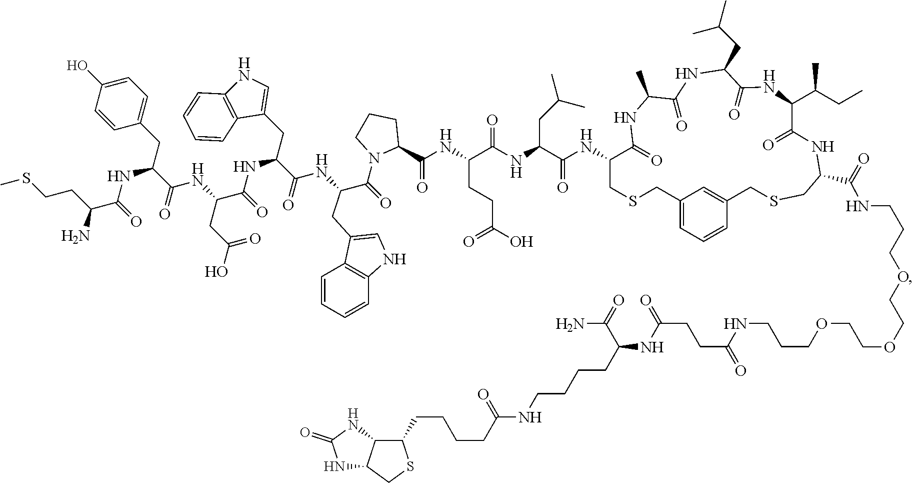 [SMILES: CC[C@H](C)[C@@H]1NC(=O)[C@H](CC(C)C)NC(=O)[C@H](C)NC(=O)[C@@H](NC(=O)[C@H](CC(C)C)NC(=O)[C@H](CCC(=O)O)NC(=O)[C@@H]2CCCN2C(=O)[C@H](Cc2c[nH]c3ccccc23)NC(=O)[C@H](Cc2c[nH]c3ccccc23)NC(=O)[C@H](CC(=O)O)NC(=O)[C@H](Cc2ccc(O)cc2)NC(=O)[C@@H](N)CCSC)CSCc2cccc(c2)CSC[C@@H](C(=O)NCCCOCCOCCOCCCNC(=O)CCC(=O)N[C@@H](CCCCNC(=O)CCCC[C@@H]2SC[C@@H]3NC(=O)N[C@@H]32)C(N)=O)NC1=O]

compound H-Met-Tyr-Asp-Trp-Trp-Pro-Glu-Leu-[Cys(3MeBn)-Ala-Leu-Ile-Cys]-Ttds-Lys(Cy5SO3)-NH₂ (GIP-058) of the following formula

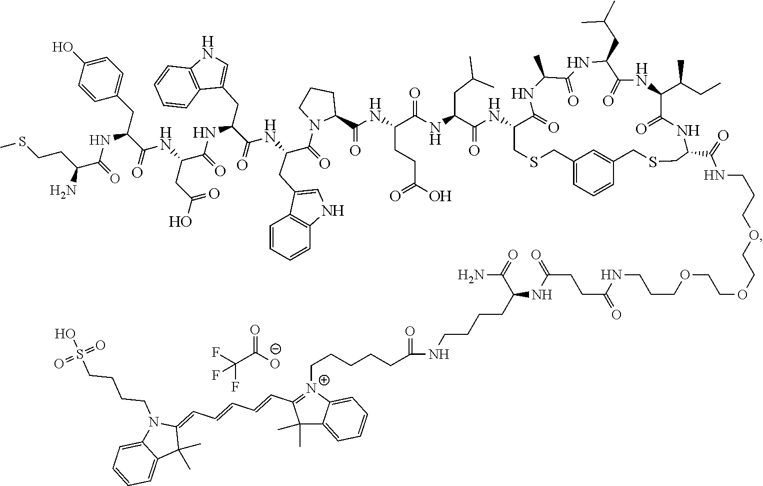 [SMILES: CC[C@H](C)[C@@H]1NC(=O)[C@H](CC(C)C)NC(=O)[C@H](C)NC(=O)[C@@H](NC(=O)[C@H](CC(C)C)NC(=O)[C@H](CCC(=O)O)NC(=O)[C@@H]2CCCN2C(=O)[C@H](Cc2c[nH]c3ccccc23)NC(=O)[C@H](Cc2c[nH]c3ccccc23)NC(=O)[C@H](CC(=O)O)NC(=O)[C@H](Cc2ccc(O)cc2)NC(=O)[C@@H](N)CCSC)CSCc2cccc(c2)CSC[C@@H](C(=O)NCCCOCCOCCOCCCNC(=O)CCC(=O)N[C@@H](CCCCNC(=O)CCCCC[N+]2=C(/C=C/C=C/C=C3/N(CCCCS(=O)(=O)O)c4ccccc4C3(C)C)C(C)(C)c3ccccc32)C(N)=O)NC1=O ; O=C([O-])C(F)(F)F]

compound Ac-Asp-Trp-Egn-Hyp-Glu-leu-[Cys(2Lut)-Thp-Leu-Ile-Cys]-NH₂ (GIP-264) of the following formula

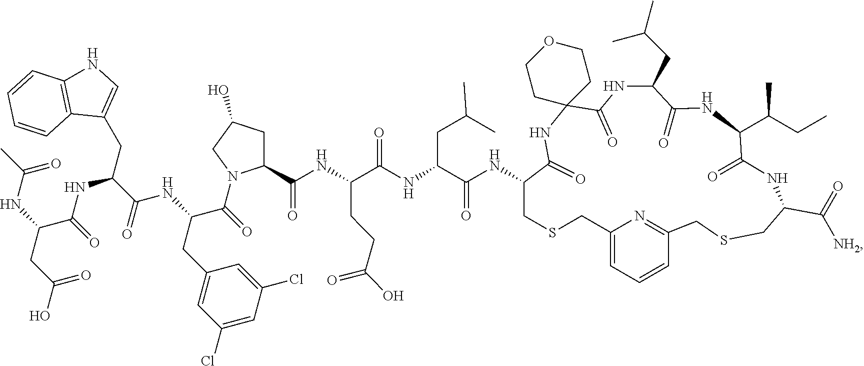 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2cc(Cl)cc(Cl)c2)NC(=O)[C@H](Cc2c[nH]c3ccccc23)NC(=O)[C@H](CC(=O)O)NC(C)=O)CSCc2cccc(n2)CSC[C@@H](C(N)=O)NC1=O]

compound DOTA-Ttds-Asp-Trp-Trp-Pro-Glu-leu-[Cys(3MeBn)-ala-Leu-Ile-Cys]-NH₂ (GIP-268) of the following formula

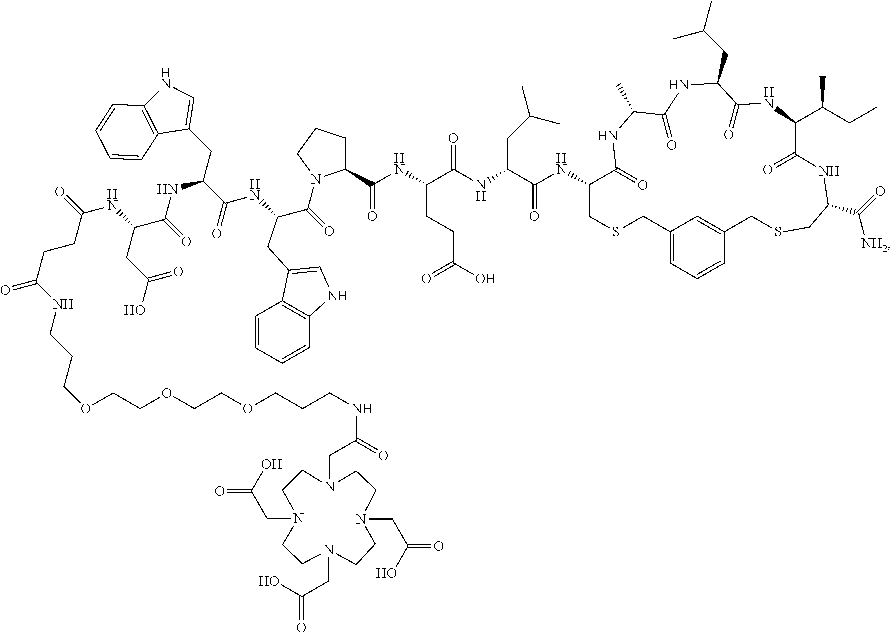 [SMILES: CC[C@H](C)[C@@H]1NC(=O)[C@H](CC(C)C)NC(=O)[C@@H](C)NC(=O)[C@@H](NC(=O)[C@@H](CC(C)C)NC(=O)[C@H](CCC(=O)O)NC(=O)[C@@H]2CCCN2C(=O)[C@H](Cc2c[nH]c3ccccc23)NC(=O)[C@H](Cc2c[nH]c3ccccc23)NC(=O)[C@H](CC(=O)O)NC(=O)CCC(=O)NCCCOCCOCCOCCCNC(=O)CN2CCN(CC(=O)O)CCN(CC(=O)O)CCN(CC(=O)O)CC2)CSCc2cccc(c2)CSC[C@@H](C(N)=O)NC1=O]

compound DOTA-Ttds-Asp-Trp-Trp-Pro-glu-Leu-[Cys(3MeBn)-ala-Leu-Ile-Cys]-NH₂ (GIP-269) of the following formula

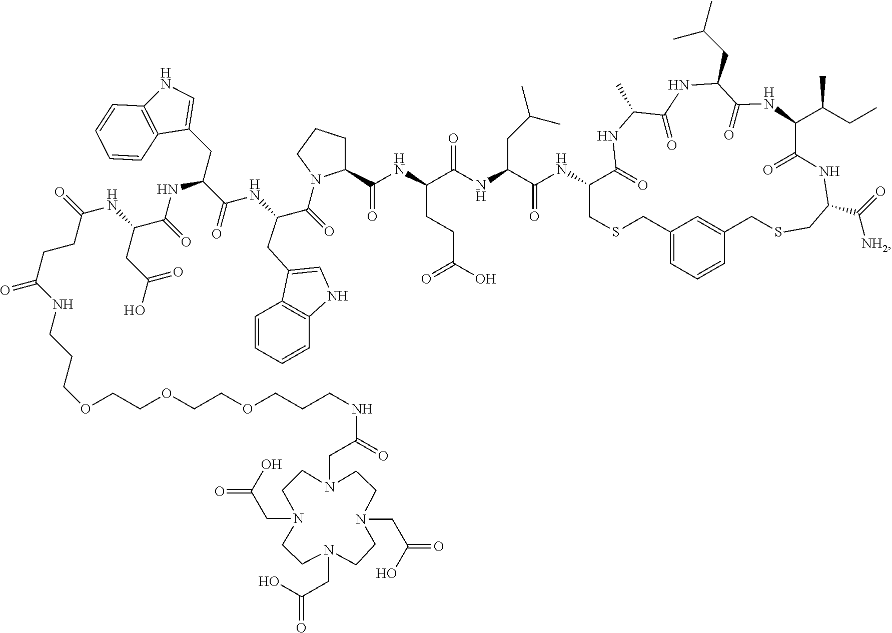 [SMILES: CC[C@H](C)[C@@H]1NC(=O)[C@H](CC(C)C)NC(=O)[C@@H](C)NC(=O)[C@@H](NC(=O)[C@H](CC(C)C)NC(=O)[C@@H](CCC(=O)O)NC(=O)[C@@H]2CCCN2C(=O)[C@H](Cc2c[nH]c3ccccc23)NC(=O)[C@H](Cc2c[nH]c3ccccc23)NC(=O)[C@H](CC(=O)O)NC(=O)CCC(=O)NCCCOCCOCCOCCCNC(=O)CN2CCN(CC(=O)O)CCN(CC(=O)O)CCN(CC(=O)O)CC2)CSCc2cccc(c2)CSC[C@@H](C(N)=O)NC1=O]

compound Ac-Asp-2Ni-Trp-Pro-Glu-Leu-[Cys(3MeBn)-Aib-Leu-Ile-Cys]-Ttds-Lys(DOTA)-NH₂ (GIP-272) of the following formula

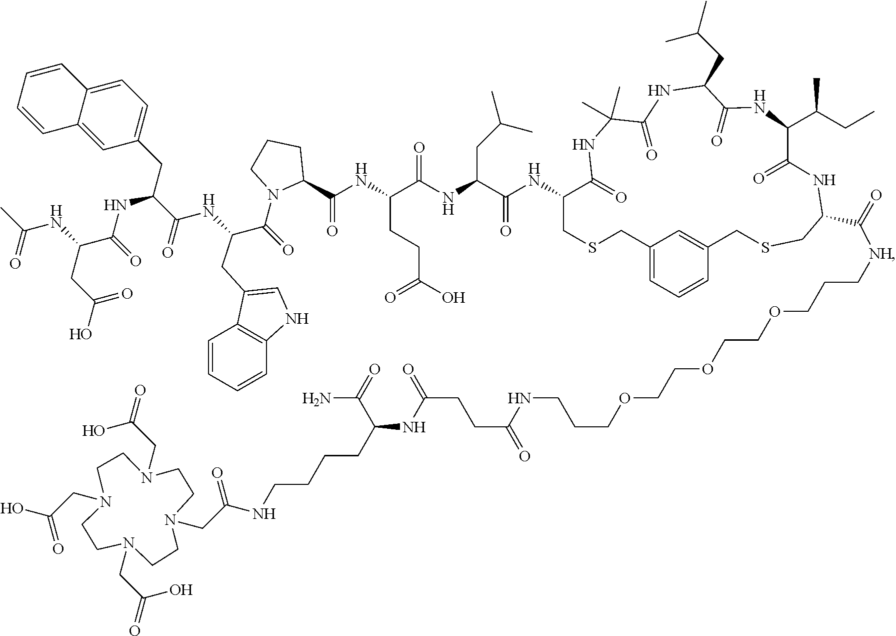 [SMILES: CC[C@H](C)[C@@H]1NC(=O)[C@H](CC(C)C)NC(=O)C(C)(C)NC(=O)[C@@H](NC(=O)[C@H](CC(C)C)NC(=O)[C@H](CCC(=O)O)NC(=O)[C@@H]2CCCN2C(=O)[C@H](Cc2c[nH]c3ccccc23)NC(=O)[C@H](Cc2ccc3ccccc3c2)NC(=O)[C@H](CC(=O)O)NC(C)=O)CSCc2cccc(c2)CSC[C@@H](C(=O)NCCCOCCOCCOCCCNC(=O)CCC(=O)N[C@@H](CCCCNC(=O)CN2CCN(CC(=O)O)CCN(CC(=O)O)CCN(CC(=O)O)CC2)C(N)=O)NC1=O]

compound Ac-Asp-Trp-Trp-Pro-Glu-Tle-[Cys(3MeBn)-Aib-Leu-Ile-Cys]-Ttds-Lys(DOTA)-NH₂ (GIP-275) of the following formula

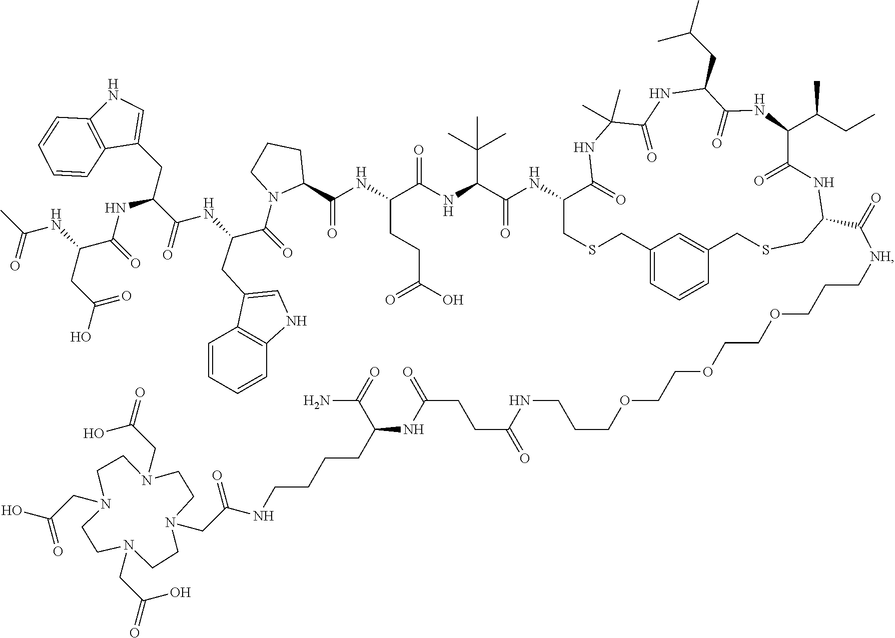 [SMILES: CC[C@H](C)[C@@H]1NC(=O)[C@H](CC(C)C)NC(=O)C(C)(C)NC(=O)[C@@H](NC(=O)[C@@H](NC(=O)[C@H](CCC(=O)O)NC(=O)[C@@H]2CCCN2C(=O)[C@H](Cc2c[nH]c3ccccc23)NC(=O)[C@H](Cc2c[nH]c3ccccc23)NC(=O)[C@H](CC(=O)O)NC(C)=O)C(C)(C)C)CSCc2cccc(c2)CSC[C@@H](C(=O)NCCCOCCOCCOCCCNC(=O)CCC(=O)N[C@@H](CCCCNC(=O)CN2CCN(CC(=O)O)CCN(CC(=O)O)CCN(CC(=O)O)CC2)C(N)=O)NC1=O]

compound Ac-Asp-Trp-Trp-Pro-Glu-Leu-[Cys(3MeBn)-Aib-Leu-Cha-Cys]-Ttds-Lys(DOTA)-NH₂ (GIP-278) of the following formula

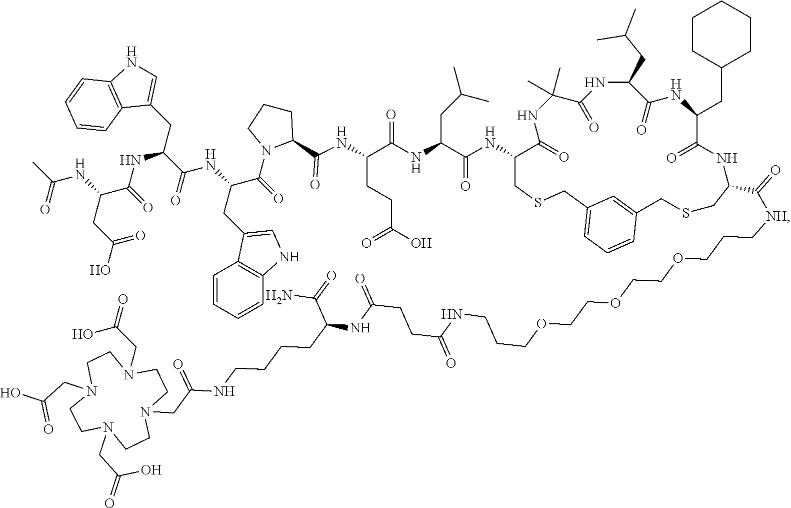 [SMILES: CC(=O)N[C@@H](CC(=O)O)C(=O)N[C@@H](Cc1c[nH]c2ccccc12)C(=O)N[C@@H](Cc1c[nH]c2ccccc12)C(=O)N1CCC[C@H]1C(=O)N[C@@H](CCC(=O)O)C(=O)N[C@@H](CC(C)C)C(=O)N[C@H]1CSCc2cccc(c2)CSC[C@@H](C(=O)NCCCOCCOCCOCCCNC(=O)CCC(=O)N[C@@H](CCCCNC(=O)CN2CCN(CC(=O)O)CCN(CC(=O)O)CCN(CC(=O)O)CC2)C(N)=O)NC(=O)[C@H](CC2CCCCC2)NC(=O)[C@H](CC(C)C)NC(=O)C(C)(C)NC1=O]

compound Nph1p-Asp-Trp-Trp-Pro-Glu-leu-[Cys(3MeBn)-Aib-Leu-Ile-Cys]-Ttds-Lys(DOTA)-NH₂ (GIP-280) of the following formula

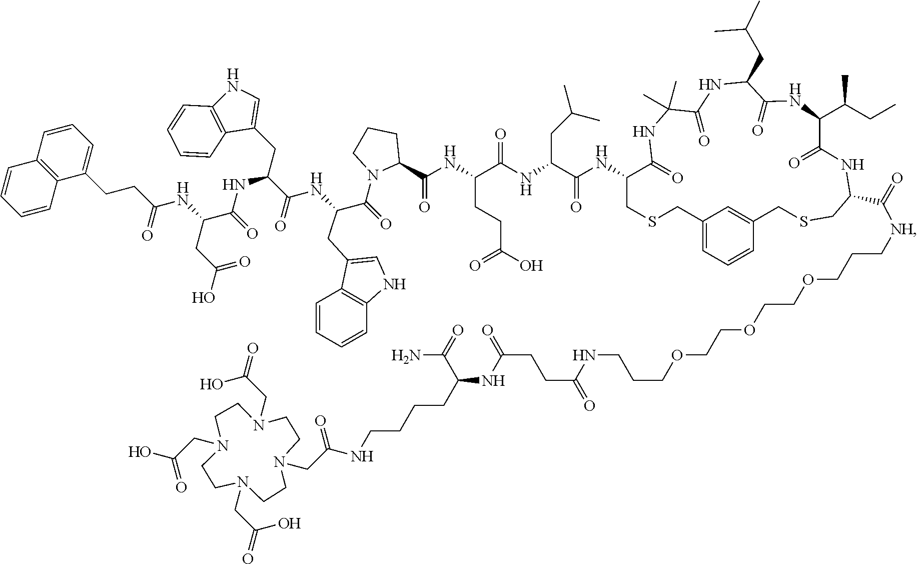 [SMILES: CC[C@H](C)[C@@H]1NC(=O)[C@H](CC(C)C)NC(=O)C(C)(C)NC(=O)[C@@H](NC(=O)[C@@H](CC(C)C)NC(=O)[C@H](CCC(=O)O)NC(=O)[C@@H]2CCCN2C(=O)[C@H](Cc2c[nH]c3ccccc23)NC(=O)[C@H](Cc2c[nH]c3ccccc23)NC(=O)[C@H](CC(=O)O)NC(=O)CCc2cccc3ccccc23)CSCc2cccc(c2)CSC[C@@H](C(=O)NCCCOCCOCCOCCCNC(=O)CCC(=O)N[C@@H](CCCCNC(=O)CN2CCN(CC(=O)O)CCN(CC(=O)O)CCN(CC(=O)O)CC2)C(N)=O)NC1=O]

compound DOTA-APAc-Asp-Gly-5Clw-Hyp-Glu-leu-[Cys(2Lut)-Thp-Leu-Ile-AET] (GIP-284) of the following formula

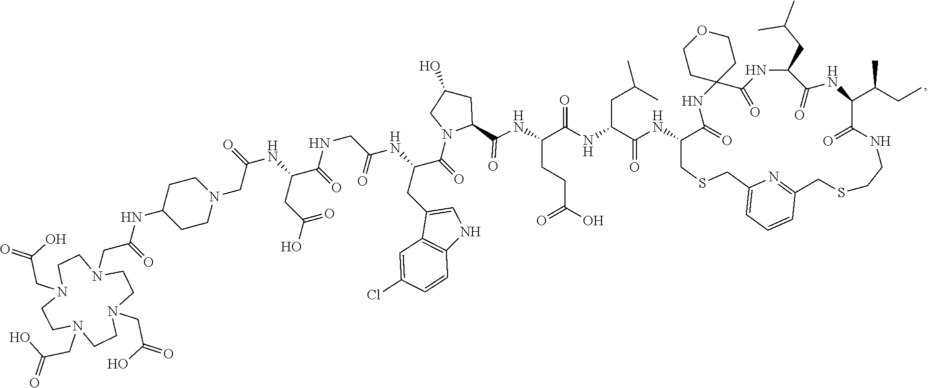 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)CNC(=O)[C@H](CC(=O)O)NC(=O)CN2CCC(NC(=O)CN3CCN(CC(=O)O)CCN(CC(=O)O)CCN(CC(=O)O)CC3)CC2)CSCc2cccc(n2)CSCCNC1=O]

compound DOTA-Ttds-Asp-Trp-Trp-Pro-Glu-leu-[Cys(3MeBn)-ala-Leu-Ile-Cys]-OH (GIP-285) of the following formula

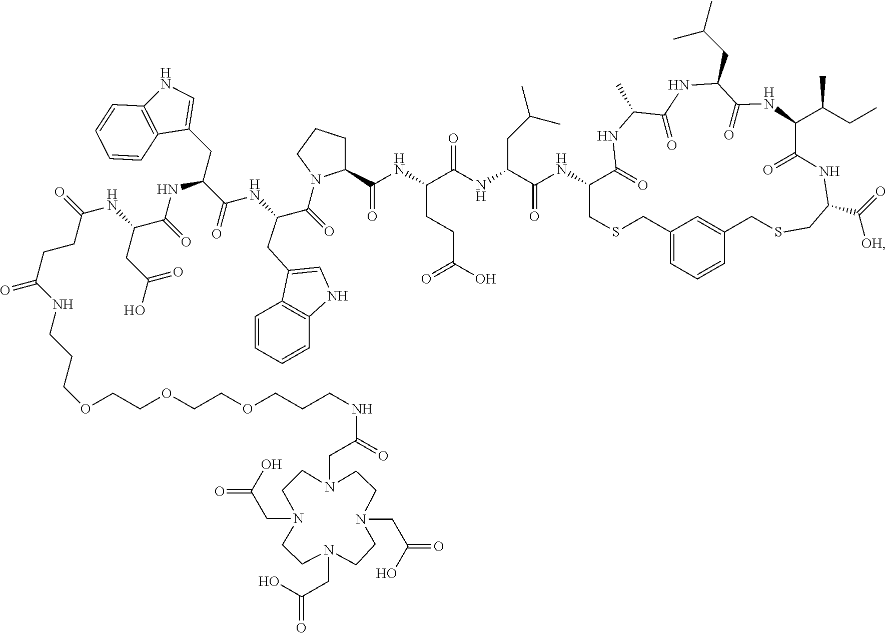 [SMILES: CC[C@H](C)[C@@H]1NC(=O)[C@H](CC(C)C)NC(=O)[C@@H](C)NC(=O)[C@@H](NC(=O)[C@@H](CC(C)C)NC(=O)[C@H](CCC(=O)O)NC(=O)[C@@H]2CCCN2C(=O)[C@H](Cc2c[nH]c3ccccc23)NC(=O)[C@H](Cc2c[nH]c3ccccc23)NC(=O)[C@H](CC(=O)O)NC(=O)CCC(=O)NCCCOCCOCCOCCCNC(=O)CN2CCN(CC(=O)O)CCN(CC(=O)O)CCN(CC(=O)O)CC2)CSCc2cccc(c2)CSC[C@@H](C(=O)O)NC1=O]

compound HO-Succinyl-Trp-Trp-Pro-Glu-leu-[Cys(tMeBn(DOTA-PP))-ala-Leu-Ile-Cys]-NH₂ (GIP-286) of the following formula

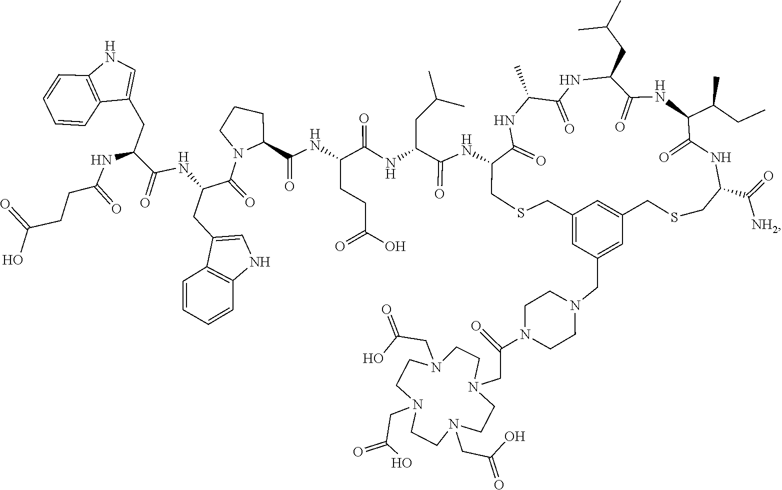 [SMILES: CC[C@H](C)[C@@H]1NC(=O)[C@H](CC(C)C)NC(=O)[C@@H](C)NC(=O)[C@@H](NC(=O)[C@@H](CC(C)C)NC(=O)[C@H](CCC(=O)O)NC(=O)[C@@H]2CCCN2C(=O)[C@H](Cc2c[nH]c3ccccc23)NC(=O)[C@H](Cc2c[nH]c3ccccc23)NC(=O)CCC(=O)O)CSCc2cc(cc(CN3CCN(C(=O)CN4CCN(CC(=O)O)CCN(CC(=O)O)CCN(CC(=O)O)CC4)CC3)c2)CSC[C@@H](C(N)=O)NC1=O]

compound HO-Succinyl-Trp-Trp-Pro-Glu-leu-[Cys(tMeBn(DOTA-AET))-ala-Leu-Ile-Cys]-NH₂ (GIP-287) of the following formula

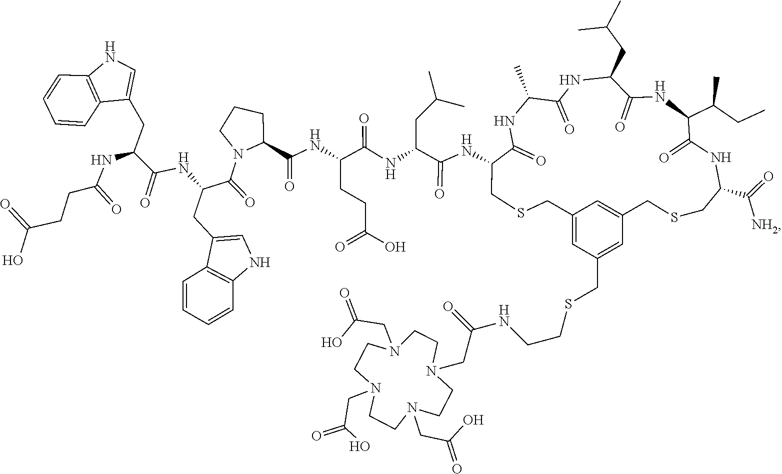 [SMILES: CC[C@H](C)[C@@H]1NC(=O)[C@H](CC(C)C)NC(=O)[C@@H](C)NC(=O)[C@@H](NC(=O)[C@@H](CC(C)C)NC(=O)[C@H](CCC(=O)O)NC(=O)[C@@H]2CCCN2C(=O)[C@H](Cc2c[nH]c3ccccc23)NC(=O)[C@H](Cc2c[nH]c3ccccc23)NC(=O)CCC(=O)O)CSCc2cc(CSCCNC(=O)CN3CCN(CC(=O)O)CCN(CC(=O)O)CCN(CC(=O)O)CC3)cc(c2)CSC[C@@H](C(N)=O)NC1=O]

compound HO-Succinyl-Trp-Trp-Pro-Glu-leu-[Cys(3MeBn)-ala-Leu-Ile-Cys]-Ttds-Lys(DOTA)-NH₂ (GIP-288) of the following formula

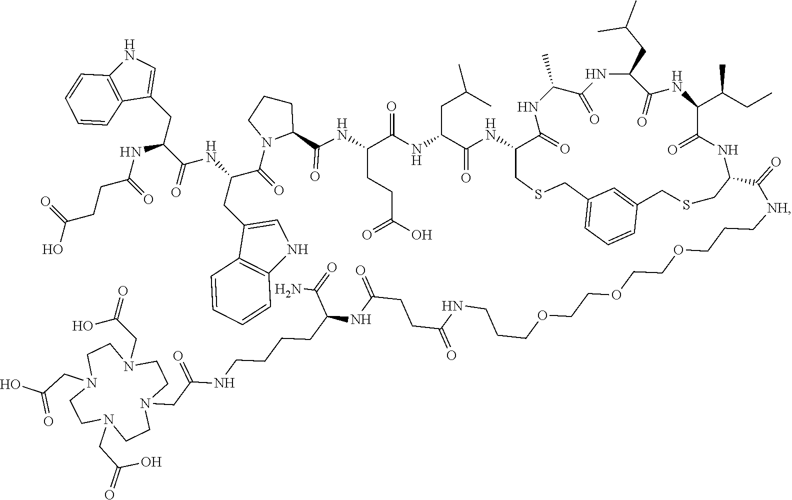 [SMILES: CC[C@H](C)[C@@H]1NC(=O)[C@H](CC(C)C)NC(=O)[C@@H](C)NC(=O)[C@@H](NC(=O)[C@@H](CC(C)C)NC(=O)[C@H](CCC(=O)O)NC(=O)[C@@H]2CCCN2C(=O)[C@H](Cc2c[nH]c3ccccc23)NC(=O)[C@H](Cc2c[nH]c3ccccc23)NC(=O)CCC(=O)O)CSCc2cccc(c2)CSC[C@@H](C(=O)NCCCOCCOCCOCCCNC(=O)CCC(=O)N[C@@H](CCCCNC(=O)CN2CCN(CC(=O)O)CCN(CC(=O)O)CCN(CC(=O)O)CC2)C(N)=O)NC1=O]

compound Ac-Asp-Trp-Nmw-Pro-Glu-Leu-[Cys(3MeBn)-Aib-Leu-Ile-Cys]-Ttds-Lys(DOTA)-NH₂ (GIP-289) of the following formula

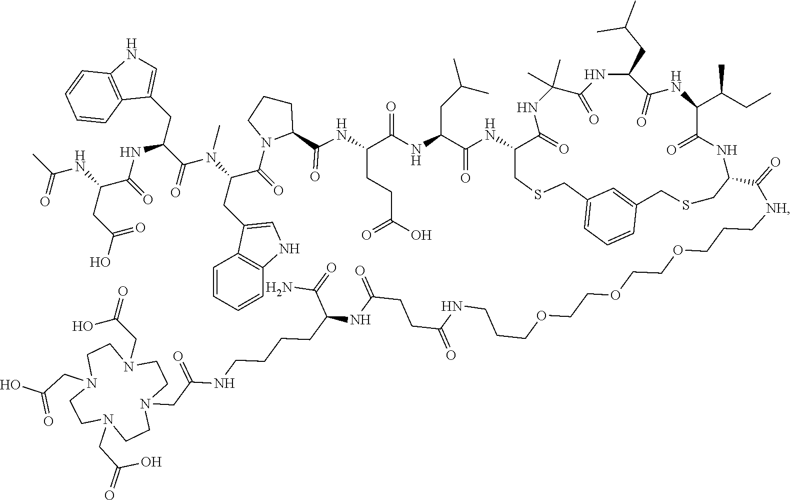 [SMILES: CC[C@H](C)[C@@H]1NC(=O)[C@H](CC(C)C)NC(=O)C(C)(C)NC(=O)[C@@H](NC(=O)[C@H](CC(C)C)NC(=O)[C@H](CCC(=O)O)NC(=O)[C@@H]2CCCN2C(=O)[C@H](Cc2c[nH]c3ccccc23)N(C)C(=O)[C@H](Cc2c[nH]c3ccccc23)NC(=O)[C@H](CC(=O)O)NC(C)=O)CSCc2cccc(c2)CSC[C@@H](C(=O)NCCCOCCOCCOCCCNC(=O)CCC(=O)N[C@@H](CCCCNC(=O)CN2CCN(CC(=O)O)CCN(CC(=O)O)CCN(CC(=O)O)CC2)C(N)=O)NC1=O]

compound Ac-Asp-Trp-Trp-Pro-Glu-Nml-[Cys(3MeBn)-Aib-Leu-Ile-Cys]-Ttds-Lys(DOTA)-NH₂ (GIP-290) of the following formula

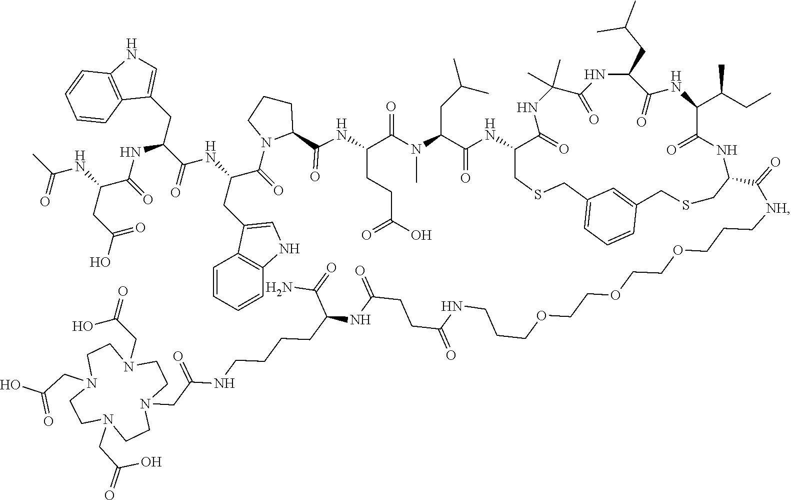 [SMILES: CC[C@H](C)[C@@H]1NC(=O)[C@H](CC(C)C)NC(=O)C(C)(C)NC(=O)[C@@H](NC(=O)[C@H](CC(C)C)N(C)C(=O)[C@H](CCC(=O)O)NC(=O)[C@@H]2CCCN2C(=O)[C@H](Cc2c[nH]c3ccccc23)NC(=O)[C@H](Cc2c[nH]c3ccccc23)NC(=O)[C@H](CC(=O)O)NC(C)=O)CSCc2cccc(c2)CSC[C@@H](C(=O)NCCCOCCOCCOCCCNC(=O)CCC(=O)N[C@@H](CCCCNC(=O)CN2CCN(CC(=O)O)CCN(CC(=O)O)CCN(CC(=O)O)CC2)C(N)=O)NC1=O]

compound Ac-Asp-Trp-Trp-Pro-Glu-Leu-[Nmc(3MeBn)-Aib-Leu-Ile-Cys]-Ttds-Lys(DOTA)-NH₂ (GIP-291) of the following formula

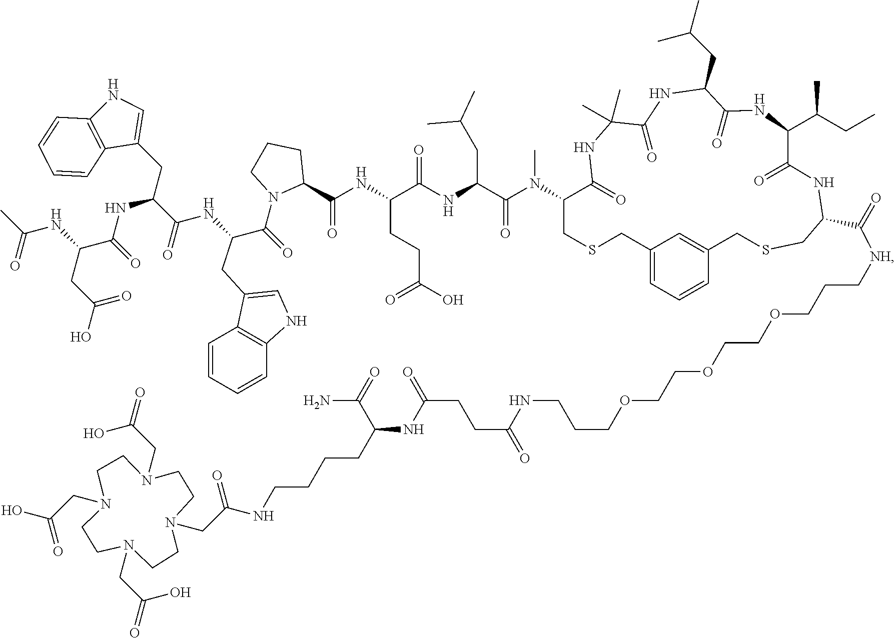 [SMILES: CC[C@H](C)[C@@H]1NC(=O)[C@H](CC(C)C)NC(=O)C(C)(C)NC(=O)[C@@H](N(C)C(=O)[C@H](CC(C)C)NC(=O)[C@H](CCC(=O)O)NC(=O)[C@@H]2CCCN2C(=O)[C@H](Cc2c[nH]c3ccccc23)NC(=O)[C@H](Cc2c[nH]c3ccccc23)NC(=O)[C@H](CC(=O)O)NC(C)=O)CSCc2cccc(c2)CSC[C@@H](C(=O)NCCCOCCOCCOCCCNC(=O)CCC(=O)N[C@@H](CCCCNC(=O)CN2CCN(CC(=O)O)CCN(CC(=O)O)CCN(CC(=O)O)CC2)C(N)=O)NC1=O]

compound Ac-Asp-Trp-Trp-Pro-Glu-Leu-[Cys(3MeBn)-Aib-Leu-Ile-Nmc]-Ttds-Lys(DOTA)-NH₂ (GIP-292) of the following formula

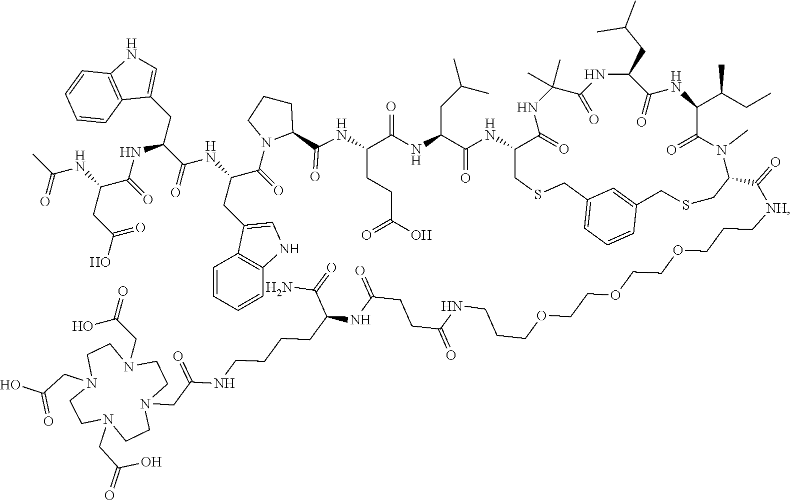 [SMILES: CC[C@H](C)[C@@H]1NC(=O)[C@H](CC(C)C)NC(=O)C(C)(C)NC(=O)[C@@H](NC(=O)[C@H](CC(C)C)NC(=O)[C@H](CCC(=O)O)NC(=O)[C@@H]2CCCN2C(=O)[C@H](Cc2c[nH]c3ccccc23)NC(=O)[C@H](Cc2c[nH]c3ccccc23)NC(=O)[C@H](CC(=O)O)NC(C)=O)CSCc2cccc(c2)CSC[C@@H](C(=O)NCCCOCCOCCOCCCNC(=O)CCC(=O)N[C@@H](CCCCNC(=O)CN2CCN(CC(=O)O)CCN(CC(=O)O)CCN(CC(=O)O)CC2)C(N)=O)N(C)C1=O]

compound Ac-Asp-1Ni-Trp-Pro-Glu-Leu-[Cys(3MeBn)-Aib-Leu-Ile-Cys]-Ttds-Lys(DOTA)-NH₂ (GIP-293) of the following formula

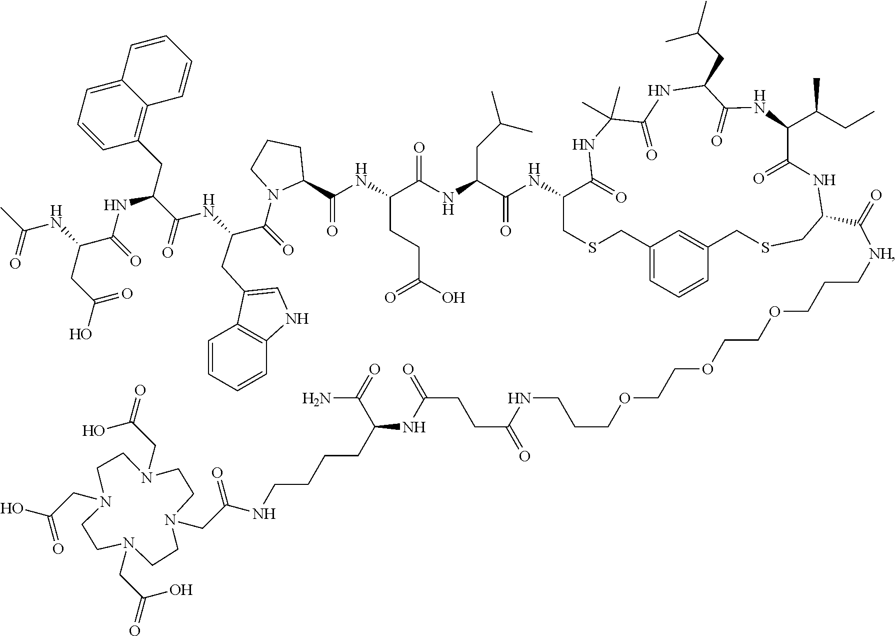 [SMILES: CC[C@H](C)[C@@H]1NC(=O)[C@H](CC(C)C)NC(=O)C(C)(C)NC(=O)[C@@H](NC(=O)[C@H](CC(C)C)NC(=O)[C@H](CCC(=O)O)NC(=O)[C@@H]2CCCN2C(=O)[C@H](Cc2c[nH]c3ccccc23)NC(=O)[C@H](Cc2cccc3ccccc23)NC(=O)[C@H](CC(=O)O)NC(C)=O)CSCc2cccc(c2)CSC[C@@H](C(=O)NCCCOCCOCCOCCCNC(=O)CCC(=O)N[C@@H](CCCCNC(=O)CN2CCN(CC(=O)O)CCN(CC(=O)O)CCN(CC(=O)O)CC2)C(N)=O)NC1=O]

compound Ac-Asp-Trp-2Ni-Pro-Glu-Leu-[Cys(3MeBn)-Aib-Leu-Ile-Cys]-Ttds-Lys(DOTA)-NH₂ (GIP-294) of the following formula

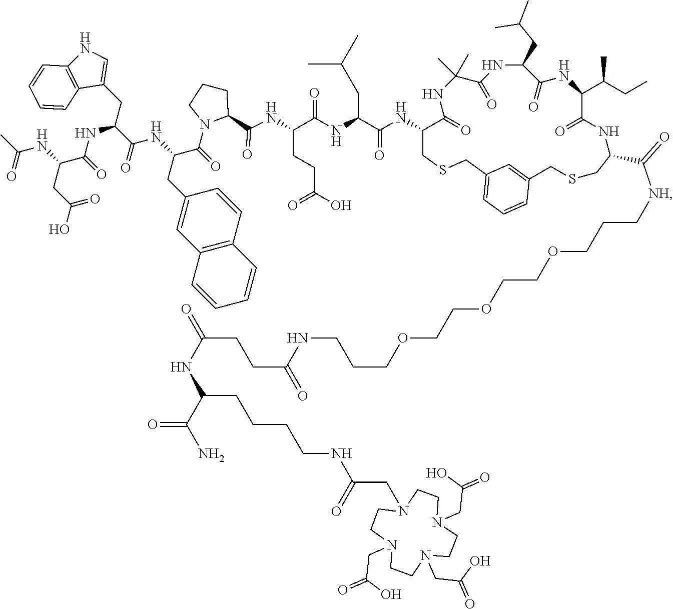

compound Ac-Asp-Trp-Trp-Pro-Glu-Cha-[Cys(3MeBn)-Aib-Leu-Ile-Cys]-Ttds-Lys(DOTA)-NH₂ (GIP-295) of the following formula

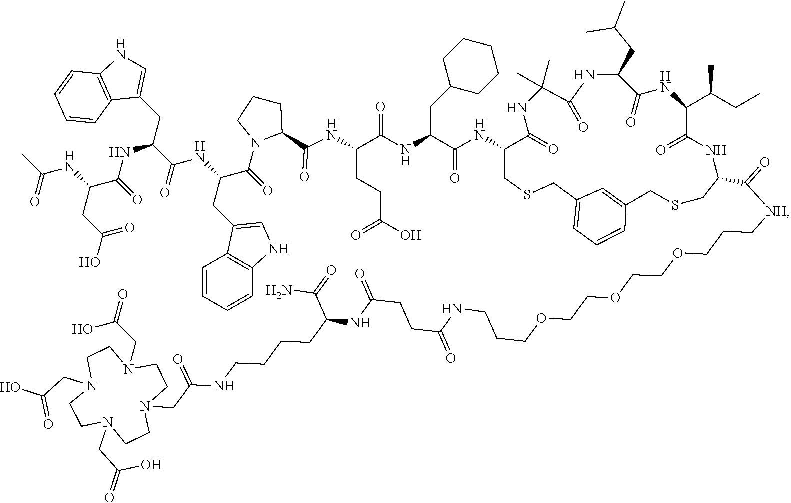 [SMILES: CC[C@H](C)[C@@H]1NC(=O)[C@H](CC(C)C)NC(=O)C(C)(C)NC(=O)[C@@H](NC(=O)[C@H](CC2CCCCC2)NC(=O)[C@H](CCC(=O)O)NC(=O)[C@@H]2CCCN2C(=O)[C@H](Cc2c[nH]c3ccccc23)NC(=O)[C@H](Cc2c[nH]c3ccccc23)NC(=O)[C@H](CC(=O)O)NC(C)=O)CSCc2cccc(c2)CSC[C@@H](C(=O)NCCCOCCOCCOCCCNC(=O)CCC(=O)N[C@@H](CCCCNC(=O)CN2CCN(CC(=O)O)CCN(CC(=O)O)CCN(CC(=O)O)CC2)C(N)=O)NC1=O]

compound Ac-Asp-Trp-Trp-Pro-Glu-Aml-[Cys(3MeBn)-Aib-Leu-Ile-Cys]-Ttds-Lys(DOTA)-NH₂ (GIP-296) of the following formula

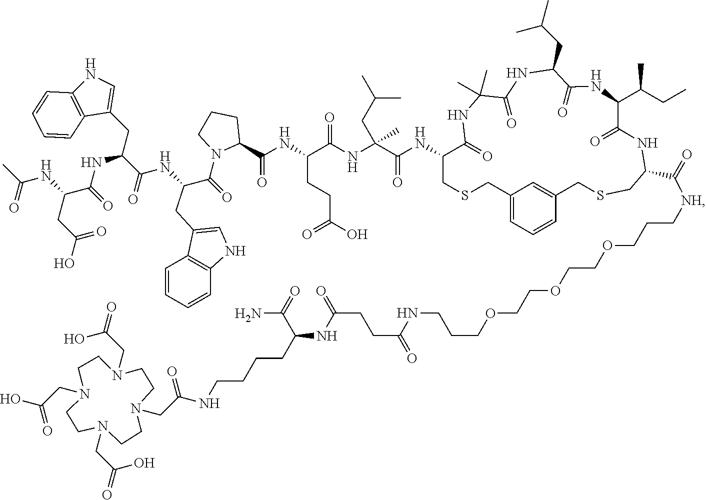 [SMILES: CC[C@H](C)[C@@H]1NC(=O)[C@H](CC(C)C)NC(=O)C(C)(C)NC(=O)[C@@H](NC(=O)[C@](C)(CC(C)C)NC(=O)[C@H](CCC(=O)O)NC(=O)[C@@H]2CCCN2C(=O)[C@H](Cc2c[nH]c3ccccc23)NC(=O)[C@H](Cc2c[nH]c3ccccc23)NC(=O)[C@H](CC(=O)O)NC(C)=O)CSCc2cccc(c2)CSC[C@@H](C(=O)NCCCOCCOCCOCCCNC(=O)CCC(=O)N[C@@H](CCCCNC(=O)CN2CCN(CC(=O)O)CCN(CC(=O)O)CCN(CC(=O)O)CC2)C(N)=O)NC1=O]

compound Ac-Asp-Nmw-Trp-Pro-Glu-Leu-[Cys(3MeBn)-Aib-Leu-Ile-Cys]-Ttds-Lys(DO TA)-NH₂ (GIP-297) of the following formula

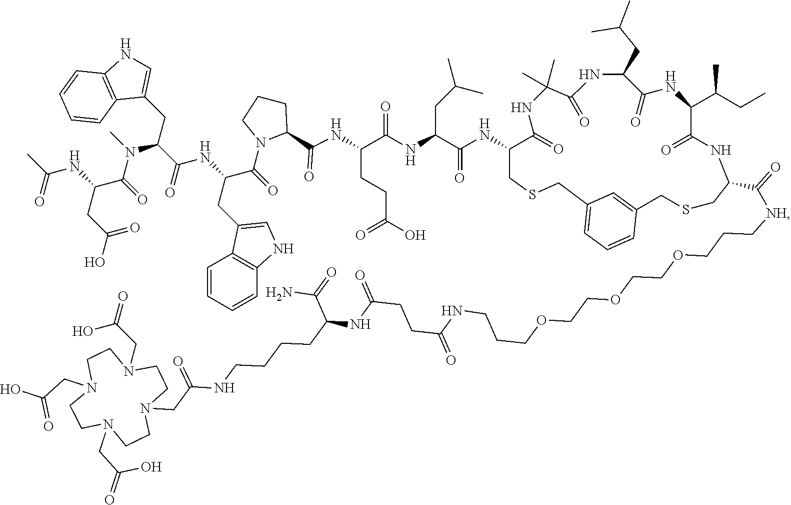 [SMILES: CC[C@H](C)[C@@H]1NC(=O)[C@H](CC(C)C)NC(=O)C(C)(C)NC(=O)[C@@H](NC(=O)[C@H](CC(C)C)NC(=O)[C@H](CCC(=O)O)NC(=O)[C@@H]2CCCN2C(=O)[C@H](Cc2c[nH]c3ccccc23)NC(=O)[C@H](Cc2c[nH]c3ccccc23)N(C)C(=O)[C@H](CC(=O)O)NC(C)=O)CSCc2cccc(c2)CSC[C@@H](C(=O)NCCCOCCOCCOCCCNC(=O)CCC(=O)N[C@@H](CCCCNC(=O)CN2CCN(CC(=O)O)CCN(CC(=O)O)CCN(CC(=O)O)CC2)C(N)=O)NC1=O]

compound Ac-1Ni-Pro-Glu-Leu-[Cys(3MeBn)-Aib-Leu-Ile-Cys]-Ttds-Lys(DOTA)-NH₂ (GIP-298) of the following formula

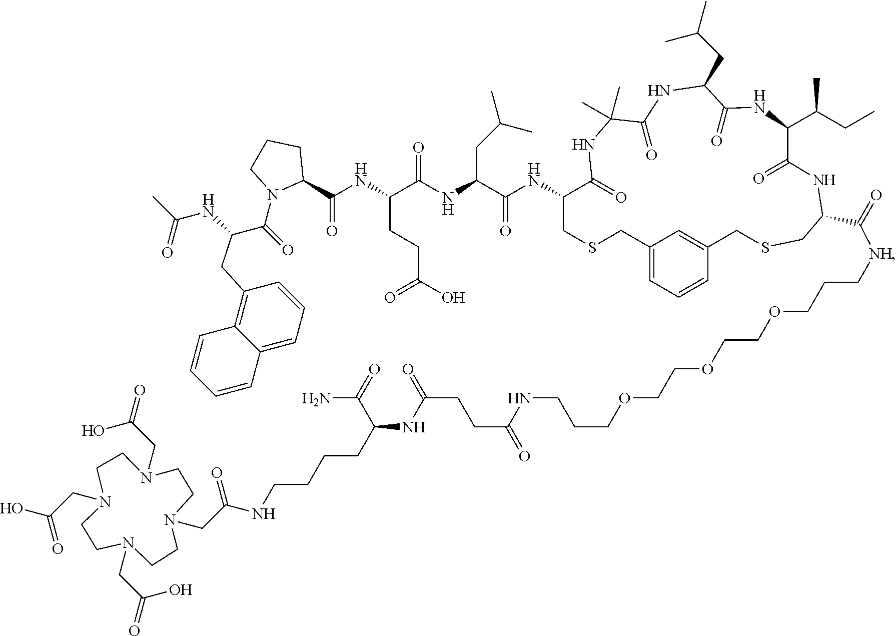 [SMILES: CC[C@H](C)[C@@H]1NC(=O)[C@H](CC(C)C)NC(=O)C(C)(C)NC(=O)[C@@H](NC(=O)[C@H](CC(C)C)NC(=O)[C@H](CCC(=O)O)NC(=O)[C@@H]2CCCN2C(=O)[C@H](Cc2cccc3ccccc23)NC(C)=O)CSCc2cccc(c2)CSC[C@@H](C(=O)NCCCOCCOCCOCCCNC(=O)CCC(=O)N[C@@H](CCCCNC(=O)CN2CCN(CC(=O)O)CCN(CC(=O)O)CCN(CC(=O)O)CC2)C(N)=O)NC1=O]

compound Nph1p-Pro-Glu-Leu-[Cys(3MeBn)-Aib-Leu-Ile-Cys]-Ttds-Lys(DOTA)-NH₂ (GIP-299) of the following formula

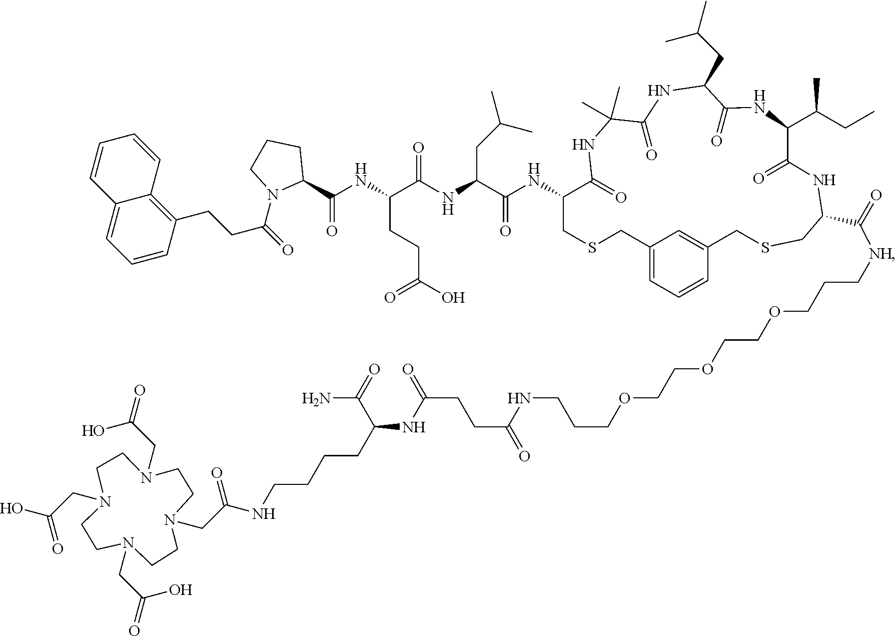 [SMILES: CC[C@H](C)[C@@H]1NC(=O)[C@H](CC(C)C)NC(=O)C(C)(C)NC(=O)[C@@H](NC(=O)[C@H](CC(C)C)NC(=O)[C@H](CCC(=O)O)NC(=O)[C@@H]2CCCN2C(=O)CCc2cccc3ccccc23)CSCc2cccc(c2)CSC[C@@H](C(=O)NCCCOCCOCCOCCCNC(=O)CCC(=O)N[C@@H](CCCCNC(=O)CN2CCN(CC(=O)O)CCN(CC(=O)O)CCN(CC(=O)O)CC2)C(N)=O)NC1=O]

compound Ac-Asp-Trp-1Ni-Pro-Glu-leu-[Cys(3MeBn)-Ala-Leu-val-Cys]-Ttds-Lys(DOTA)-NH₂ (GIP-300) of the following formula

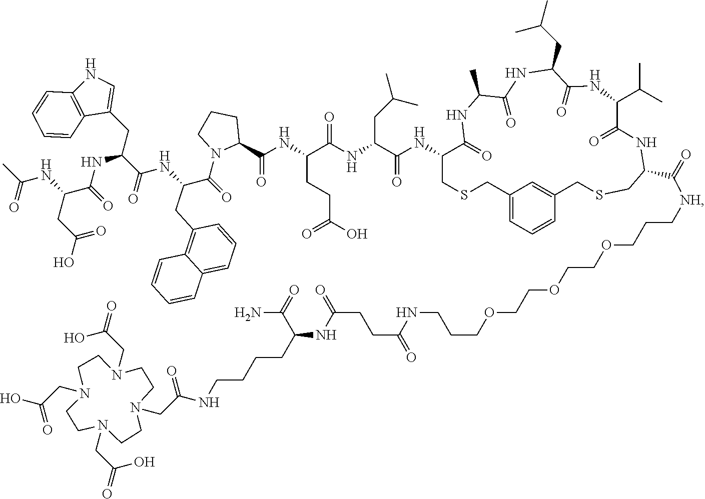 [SMILES: CC(=O)N[C@@H](CC(=O)O)C(=O)N[C@@H](Cc1c[nH]c2ccccc12)C(=O)N[C@@H](Cc1cccc2ccccc12)C(=O)N1CCC[C@H]1C(=O)N[C@@H](CCC(=O)O)C(=O)N[C@H](CC(C)C)C(=O)N[C@H]1CSCc2cccc(c2)CSC[C@@H](C(=O)NCCCOCCOCCOCCCNC(=O)CCC(=O)N[C@@H](CCCCNC(=O)CN2CCN(CC(=O)O)CCN(CC(=O)O)CCN(CC(=O)O)CC2)C(N)=O)NC(=O)[C@@H](C(C)C)NC(=O)[C@H](CC(C)C)NC(=O)[C@H](C)NC1=O]

compound Ac-Trp-1Ni-Pro-Glu-Leu-[Cys(3MeBn)-Aib-Leu-Ile-Cys]-Ttds-Lys(DOTA)-NH₂ (GIP-301) of the following formula

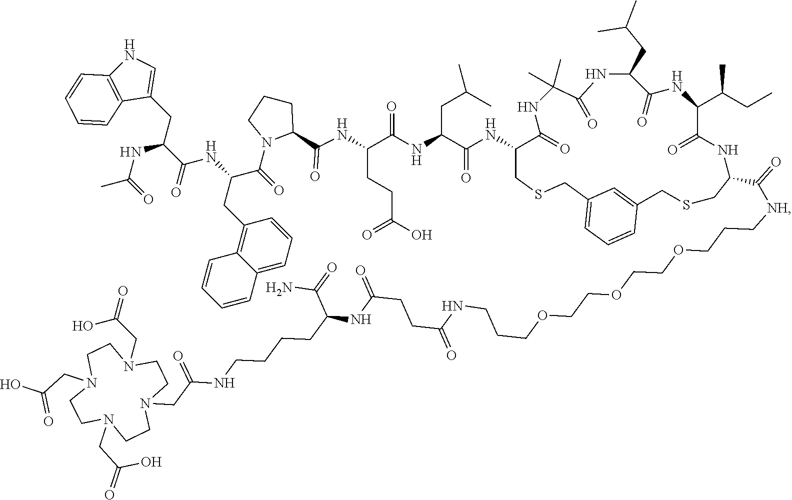 [SMILES: CC[C@H](C)[C@@H]1NC(=O)[C@H](CC(C)C)NC(=O)C(C)(C)NC(=O)[C@@H](NC(=O)[C@H](CC(C)C)NC(=O)[C@H](CCC(=O)O)NC(=O)[C@@H]2CCCN2C(=O)[C@H](Cc2cccc3ccccc23)NC(=O)[C@H](Cc2c[nH]c3ccccc23)NC(C)=O)CSCc2cccc(c2)CSC[C@@H](C(=O)NCCCOCCOCCOCCCNC(=O)CCC(=O)N[C@@H](CCCCNC(=O)CN2CCN(CC(=O)O)CCN(CC(=O)O)CCN(CC(=O)O)CC2)C(N)=O)NC1=O]

compound Ac-Asp-Trp-Hyw-Pro-Glu-leu-[Cys(3MeBn)-Aib-Leu-Ile-Cys]-Ttds-Lys(DOTA)-NH₂ (GIP-302) of the following formula

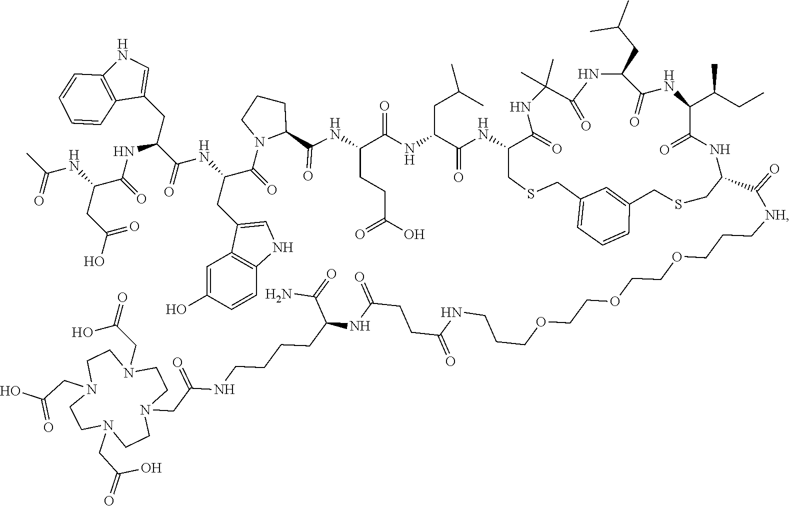 [SMILES: CC[C@H](C)[C@@H]1NC(=O)[C@H](CC(C)C)NC(=O)C(C)(C)NC(=O)[C@@H](NC(=O)[C@@H](CC(C)C)NC(=O)[C@H](CCC(=O)O)NC(=O)[C@@H]2CCCN2C(=O)[C@H](Cc2c[nH]c3ccc(O)cc23)NC(=O)[C@H](Cc2c[nH]c3ccccc23)NC(=O)[C@H](CC(=O)O)NC(C)=O)CSCc2cccc(c2)CSC[C@@H](C(=O)NCCCOCCOCCOCCCNC(=O)CCC(=O)N[C@@H](CCCCNC(=O)CN2CCN(CC(=O)O)CCN(CC(=O)O)CCN(CC(=O)O)CC2)C(N)=O)NC1=O]

compound Ac-Asp-7Nw-1Ni-Pro-Glu-leu-[Cys(3MeBn)-Aib-Leu-Ile-Cys]-Ttds-Lys(DOTA)-NH₂ (GIP-303) of the following formula

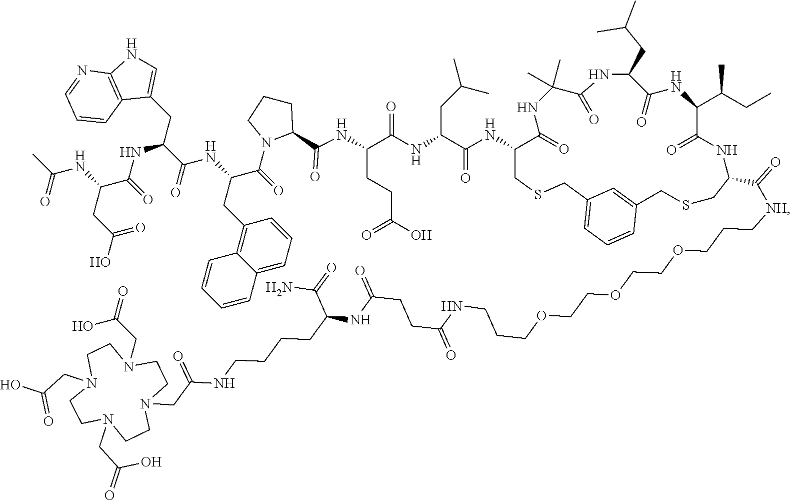 [SMILES: CC[C@H](C)[C@@H]1NC(=O)[C@H](CC(C)C)NC(=O)C(C)(C)NC(=O)[C@@H](NC(=O)[C@@H](CC(C)C)NC(=O)[C@H](CCC(=O)O)NC(=O)[C@@H]2CCCN2C(=O)[C@H](Cc2cccc3ccccc23)NC(=O)[C@H](Cc2c[nH]c3ncccc23)NC(=O)[C@H](CC(=O)O)NC(C)=O)CSCc2cccc(c2)CSC[C@@H](C(=O)NCCCOCCOCCOCCCNC(=O)CCC(=O)N[C@@H](CCCCNC(=O)CN2CCN(CC(=O)O)CCN(CC(=O)O)CCN(CC(=O)O)CC2)C(N)=O)NC1=O]

compound DOTA-Ttds-Trp-Asp-Pro-5Clw-leu-Glu-[Cys(3MeBn)-Leu-Ile-Aib-AET] (GIP-304) of the following formula

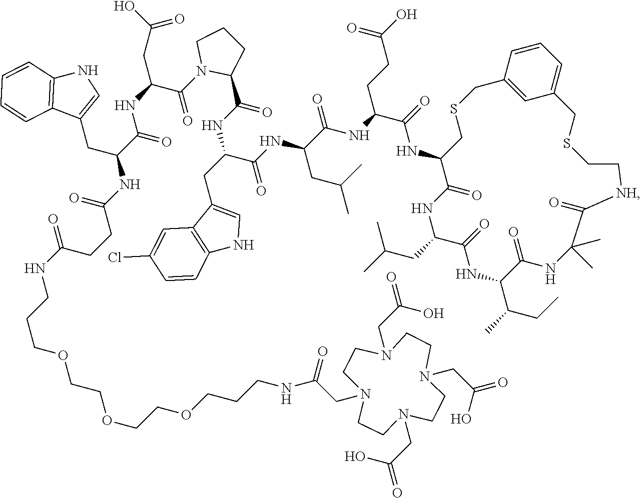 [SMILES: CC[C@H](C)[C@@H]1NC(=O)[C@H](CC(C)C)NC(=O)[C@@H](NC(=O)[C@H](CCC(=O)O)NC(=O)[C@@H](CC(C)C)NC(=O)[C@H](Cc2c[nH]c3ccc(Cl)cc23)NC(=O)[C@@H]2CCCN2C(=O)[C@H](CC(=O)O)NC(=O)[C@H](Cc2c[nH]c3ccccc23)NC(=O)CCC(=O)NCCCOCCOCCOCCCNC(=O)CN2CCN(CC(=O)O)CCN(CC(=O)O)CCN(CC(=O)O)CC2)CSCc2cccc(c2)CSCCNC(=O)C(C)(C)NC1=O]

compound DOTA-Ttds-leu-Asp-Pro-5Clw-Trp-Leu-[Cys(3MeBn)-Glu-Aib-Ile-AET] (GIP-305) of the following formula

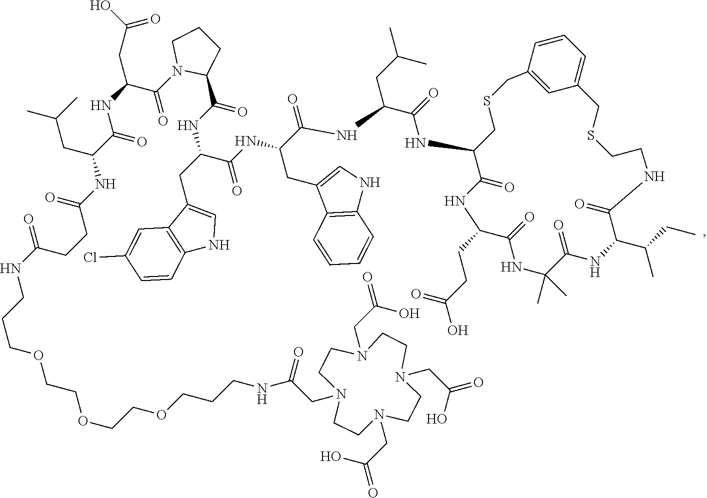 [SMILES: CC[C@H](C)[C@@H]1NC(=O)C(C)(C)NC(=O)[C@H](CCC(=O)O)NC(=O)[C@@H](NC(=O)[C@H](CC(C)C)NC(=O)[C@H](Cc2c[nH]c3ccccc23)NC(=O)[C@H](Cc2c[nH]c3ccc(Cl)cc23)NC(=O)[C@@H]2CCCN2C(=O)[C@H](CC(=O)O)NC(=O)[C@@H](CC(C)C)NC(=O)CCC(=O)NCCCOCCOCCOCCCNC(=O)CN2CCN(CC(=O)O)CCN(CC(=O)O)CCN(CC(=O)O)CC2)CSCc2cccc(c2)CSCCNC1=O]

compound DOTA-Ttds-5Clw-Pro-Glu-leu-Asp-Trp-[Cys(3MeBn)-Ile-Leu-Aib-AET] (GIP-306 of the following formula

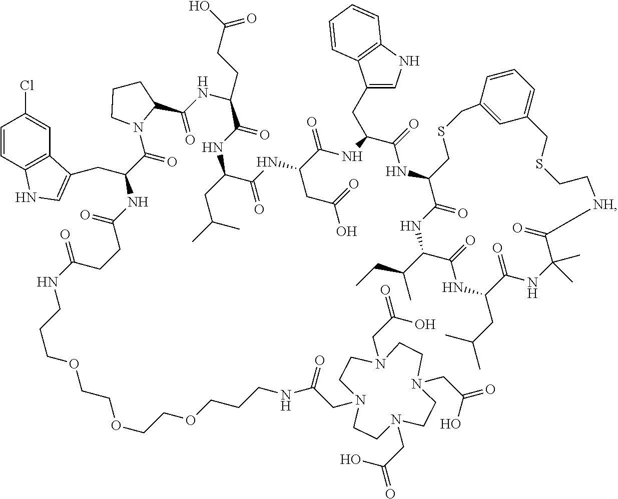

compound H-Met-Tyr-asp-Trp-Trp-Pro-Glu-Leu-[Cys(3MeBn)-Ala-Leu-Ile-Cys]-NH₂ (GIP-307) of the following formula

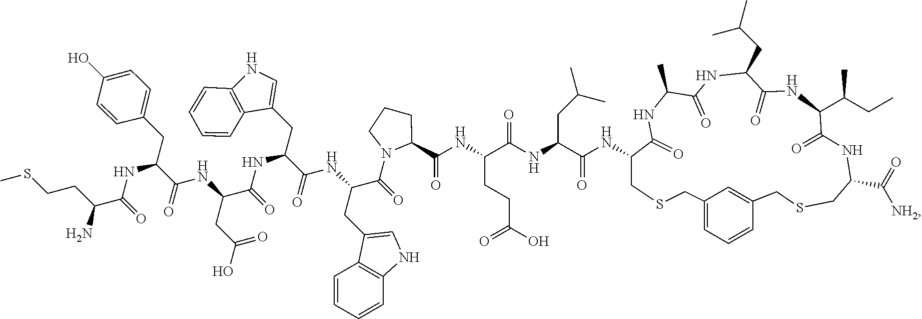 [SMILES: CC[C@H](C)[C@@H]1NC(=O)[C@H](CC(C)C)NC(=O)[C@H](C)NC(=O)[C@@H](NC(=O)[C@H](CC(C)C)NC(=O)[C@H](CCC(=O)O)NC(=O)[C@@H]2CCCN2C(=O)[C@H](Cc2c[nH]c3ccccc23)NC(=O)[C@H](Cc2c[nH]c3ccccc23)NC(=O)[C@@H](CC(=O)O)NC(=O)[C@H](Cc2ccc(O)cc2)NC(=O)[C@@H](N)CCSC)CSCc2cccc(c2)CSC[C@@H](C(N)=O)NC1=O]

compound H-Met-Tyr-Asp-trp-Trp-Pro-Glu-Leu-[Cys(3MeBn)-Ala-Leu-Ile-Cys]-NH₂ (GIP-308) of the following formula

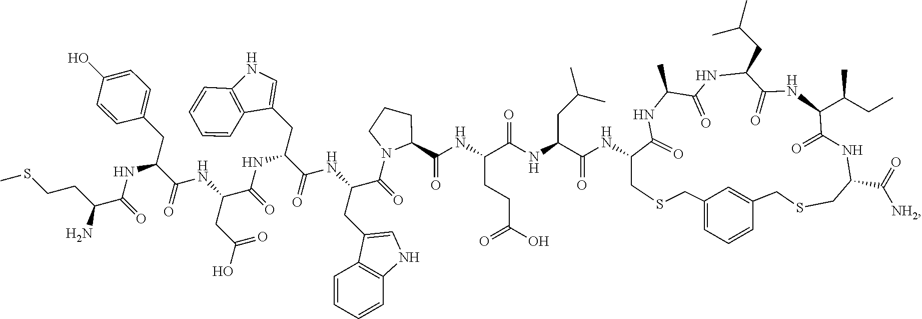 [SMILES: CC[C@H](C)[C@@H]1NC(=O)[C@H](CC(C)C)NC(=O)[C@H](C)NC(=O)[C@@H](NC(=O)[C@H](CC(C)C)NC(=O)[C@H](CCC(=O)O)NC(=O)[C@@H]2CCCN2C(=O)[C@H](Cc2c[nH]c3ccccc23)NC(=O)[C@@H](Cc2c[nH]c3ccccc23)NC(=O)[C@H](CC(=O)O)NC(=O)[C@H](Cc2ccc(O)cc2)NC(=O)[C@@H](N)CCSC)CSCc2cccc(c2)CSC[C@@H](C(N)=O)NC1=O]

compound H-Met-Tyr-Asp-Trp-Trp-Pro-Glu-Leu-[cys(3MeBn)-Ala-Leu-Ile-Cys]-NH₂ (GIP-309) of the following formula

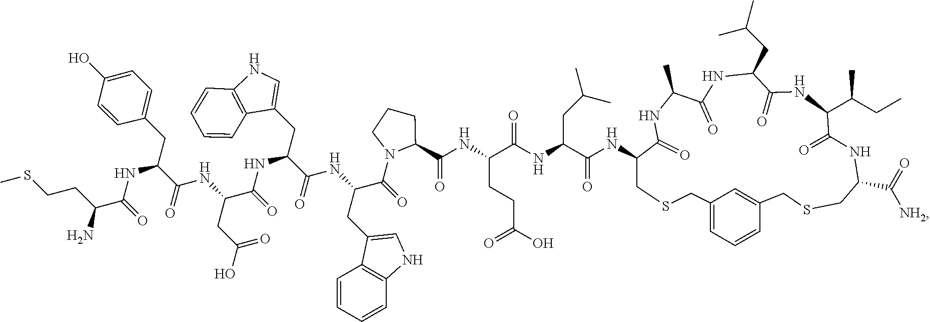

compound H-Met-Tyr-Asp-Trp-Trp-Pro-Glu-Leu-[Cys(3MeBn)-Ala-Leu-Ile-cys]-NH₂ (GIP-310) of the following formula

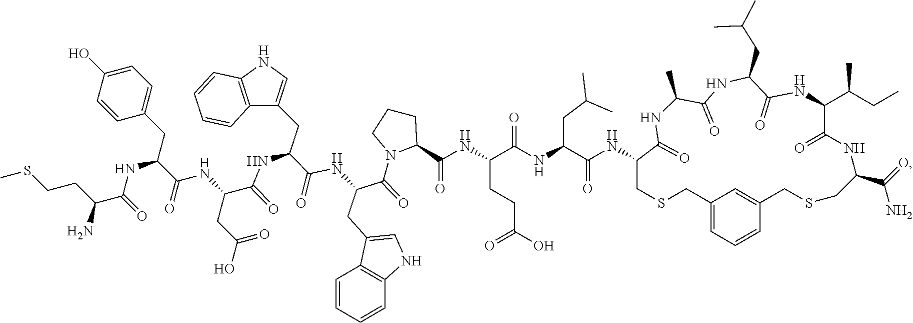

compound H-Met-Tyr-Asp-Trp-Trp-Ala-Glu-Leu-[Cys(3MeBn)-Ala-Leu-Ile-Cys]-NH₂ (GIP-311) of the following formula

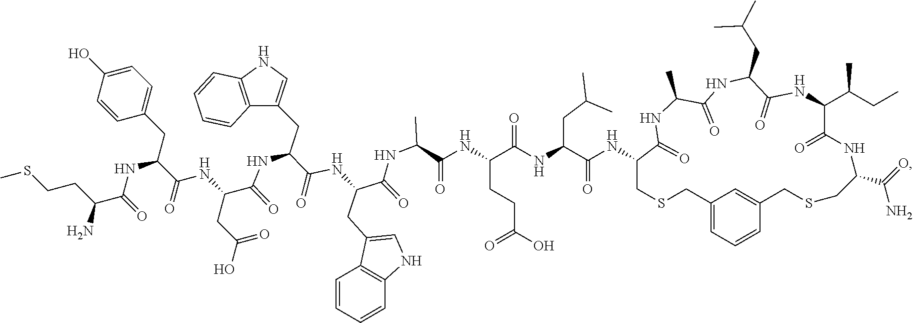

compound H-Met-Tyr-Asp-Trp-Trp-Pro-Glu-Leu-[Cys(3MeBn)-Ala-Ala-Ile-Cys]-NH₂ (GIP-312) of the following formula

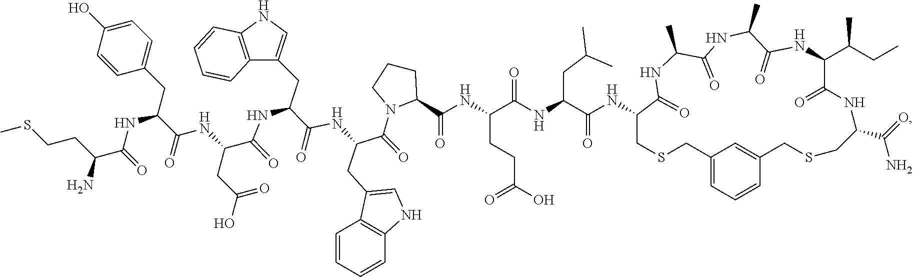

compound H-Met-Tyr-Asp-Trp-Trp-Pro-Glu-Leu-[Cys(2MeBn)-Ala-leu-Ile-Cys]-NH₂ (GIP-313) of the following formula

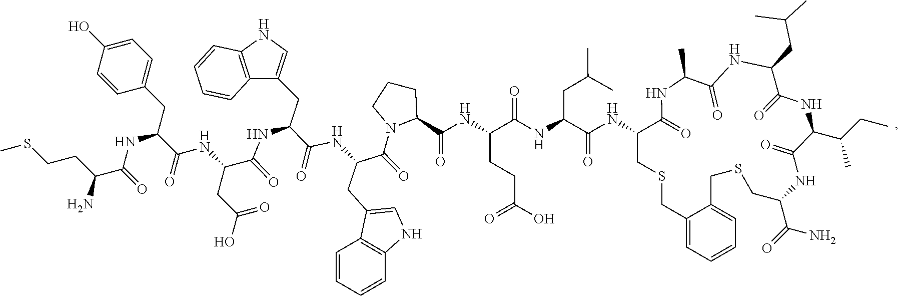

compound HO-Succinyl-Trp-Trp-Pro-Glu-Leu-[Cys(3MeBn)-ala-Leu-Ile-Cys]-NH₂ (GIP-314) of the following formula

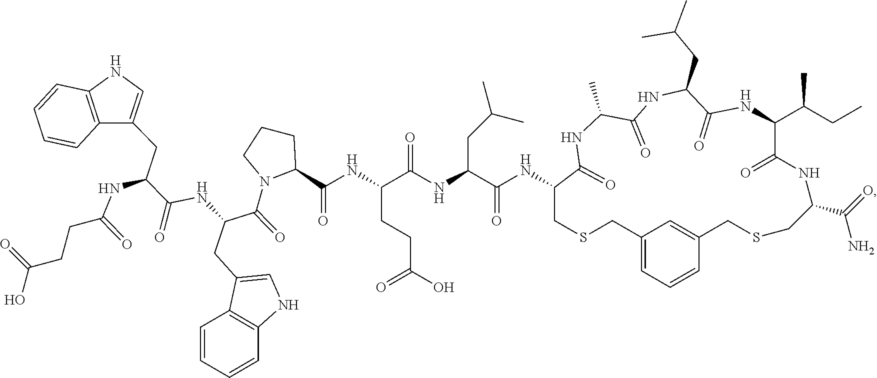 [SMILES: CC[C@H](C)[C@@H]1NC(=O)[C@H](CC(C)C)NC(=O)[C@@H](C)NC(=O)[C@@H](NC(=O)[C@H](CC(C)C)NC(=O)[C@H](CCC(=O)O)NC(=O)[C@@H]2CCCN2C(=O)[C@H](Cc2c[nH]c3ccccc23)NC(=O)[C@H](Cc2c[nH]c3ccccc23)NC(=O)CCC(=O)O)CSCc2cccc(c2)CSC[C@@H](C(N)=O)NC1=O]

compound Ac-Cya-Gly-5Clw-Hyp-Glu-leu-[Cys(2Lut)-Aib-Leu-Ile-AET] (GIP-316) of the following formula

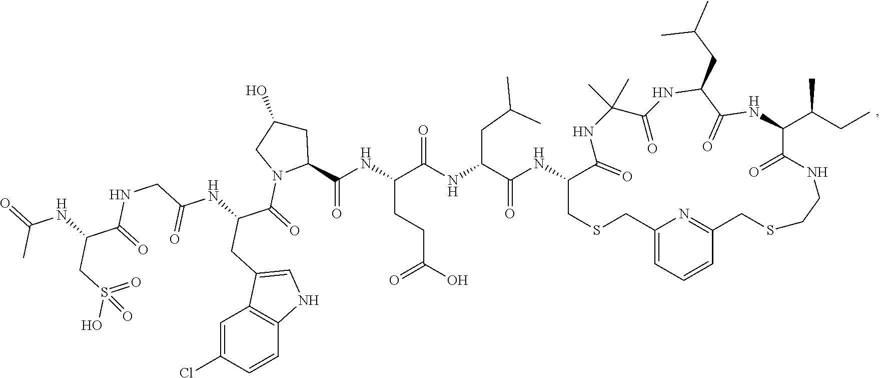 [SMILES: CC[C@H](C)[C@@H]1NC(=O)[C@H](CC(C)C)NC(=O)C(C)(C)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)CNC(=O)[C@H](CS(=O)(=O)O)NC(C)=O)CSCc2cccc(n2)CSCCNC1=O]

compound Ac-Cya-Trp-Trp-Pro-Glu-Leu-[Cys(3MeBn)-Ala-Leu-Ile-Cys]-NH₂ (GIP-317) of the following formula

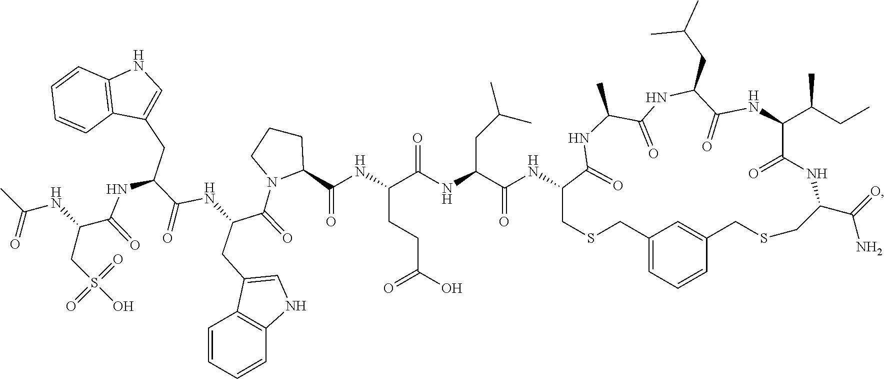 [SMILES: CC[C@H](C)[C@@H]1NC(=O)[C@H](CC(C)C)NC(=O)[C@H](C)NC(=O)[C@@H](NC(=O)[C@H](CC(C)C)NC(=O)[C@H](CCC(=O)O)NC(=O)[C@@H]2CCCN2C(=O)[C@H](Cc2c[nH]c3ccccc23)NC(=O)[C@H](Cc2c[nH]c3ccccc23)NC(=O)[C@H](CS(=O)(=O)O)NC(C)=O)CSCc2cccc(c2)CSC[C@@H](C(N)=O)NC1=O]

compound HO-Succinyl-Trp-Trp-Pro-Glu-leu-[Cys(3MeBn)-ala-Leu-Ile-Cys]-NH₂ (GIP-318) of the following formula

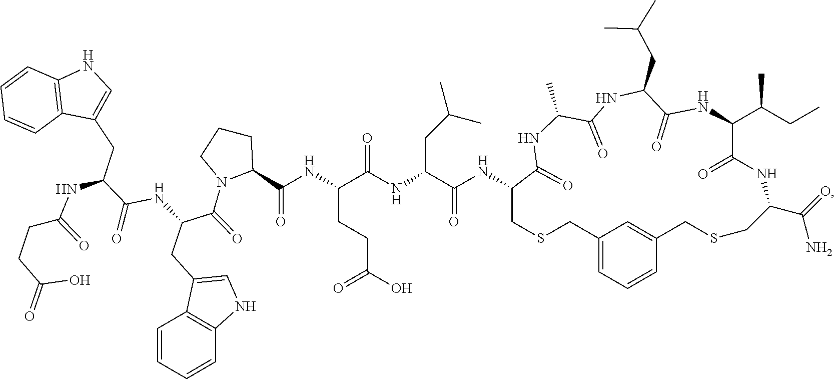 [SMILES: CC[C@H](C)[C@@H]1NC(=O)[C@H](CC(C)C)NC(=O)[C@@H](C)NC(=O)[C@@H](NC(=O)[C@@H](CC(C)C)NC(=O)[C@H](CCC(=O)O)NC(=O)[C@@H]2CCCN2C(=O)[C@H](Cc2c[nH]c3ccccc23)NC(=O)[C@H](Cc2c[nH]c3ccccc23)NC(=O)CCC(=O)O)CSCc2cccc(c2)CSC[C@@H](C(N)=O)NC1=O]

compound Ac-Asp-Trp-Mcf-Hyp-Glu-leu-[Cys(2Lut)-Thp-Leu-Ile-Cys]-NH₂ (GIP-320) of the following formula

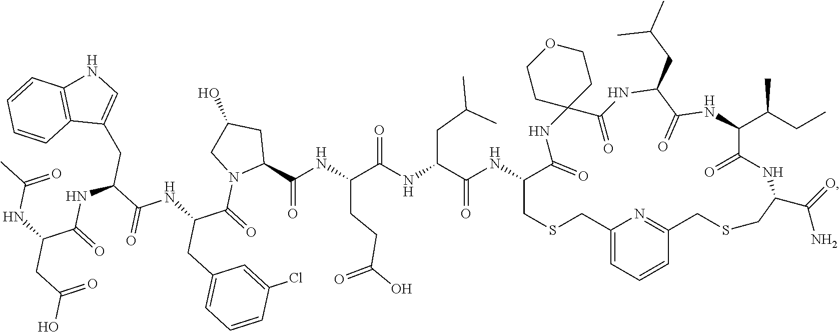 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2cccc(Cl)c2)NC(=O)[C@H](Cc2c[nH]c3ccccc23)NC(=O)[C@H](CC(=O)O)NC(C)=O)CSCc2cccc(n2)CSC[C@@H](C(N)=O)NC1=O]

compound Ac-Asp-Trp-5Clw-Hyp-Glu-leu-[Cys(2Lut)-Thp-Leu-Ser-Cys]-NH₂ (GIP-322) of the following formula

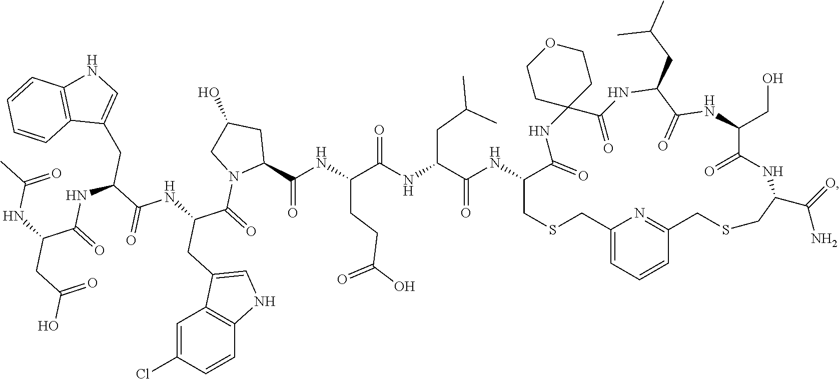 [SMILES: CC(=O)N[C@@H](CC(=O)O)C(=O)N[C@@H](Cc1c[nH]c2ccccc12)C(=O)N[C@@H](Cc1c[nH]c2ccc(Cl)cc12)C(=O)N1C[C@H](O)C[C@H]1C(=O)N[C@@H](CCC(=O)O)C(=O)N[C@H](CC(C)C)C(=O)N[C@H]1CSCc2cccc(n2)CSC[C@@H](C(N)=O)NC(=O)[C@H](CO)NC(=O)[C@H](CC(C)C)NC(=O)C2(CCOCC2)NC1=O]

compound Ac-Asp-Eaa-Eaa-Hyp-Glu-leu-[Cys(2Lut)-Thp-Leu-Ile-Cys]-NH₂ (GIP-323) of the following formula

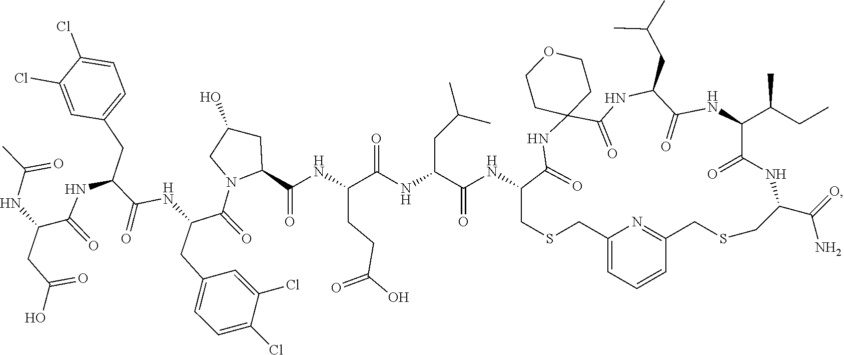 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2ccc(Cl)c(Cl)c2)NC(=O)[C@H](Cc2ccc(Cl)c(Cl)c2)NC(=O)[C@H](CC(=O)O)NC(C)=O)CSCc2cccc(n2)CSC[C@@H](C(N)=O)NC1=O]

compound Ac-Trp-Trp-Pro-Glu-Leu-[Cys(3MeBn)-Ala-Leu-Ile-Cys]-NH₂ (GIP-324) of the following formula

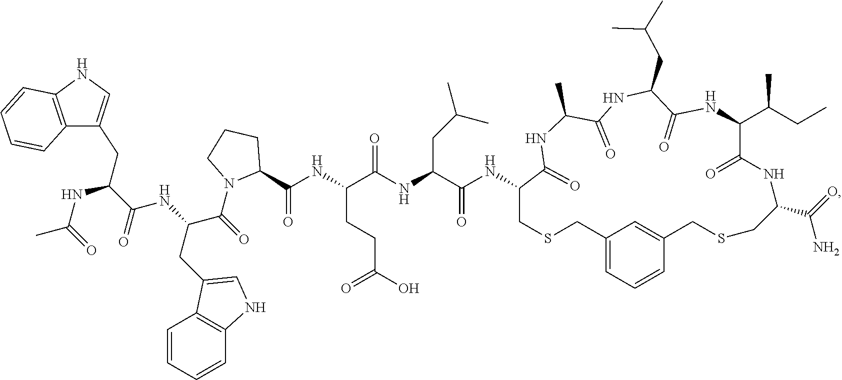 [SMILES: CC[C@H](C)[C@@H]1NC(=O)[C@H](CC(C)C)NC(=O)[C@H](C)NC(=O)[C@@H](NC(=O)[C@H](CC(C)C)NC(=O)[C@H](CCC(=O)O)NC(=O)[C@@H]2CCCN2C(=O)[C@H](Cc2c[nH]c3ccccc23)NC(=O)[C@H](Cc2c[nH]c3ccccc23)NC(C)=O)CSCc2cccc(c2)CSC[C@@H](C(N)=O)NC1=O]

compound Ac-Trp-Pro-Glu-Leu-[Cys(3MeBn)-Ala-Leu-Ile-Cys]-NH₂ (GIP-325) of the following formula

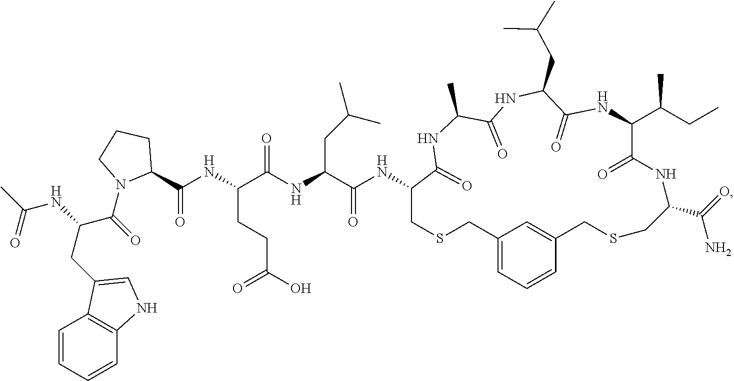 [SMILES: CC[C@H](C)[C@@H]1NC(=O)[C@H](CC(C)C)NC(=O)[C@H](C)NC(=O)[C@@H](NC(=O)[C@H](CC(C)C)NC(=O)[C@H](CCC(=O)O)NC(=O)[C@@H]2CCCN2C(=O)[C@H](Cc2c[nH]c3ccccc23)NC(C)=O)CSCc2cccc(c2)CSC[C@@H](C(N)=O)NC1=O]

compound Ac-Pro-Glu-Leu-[Cys(3MeBn)-Ala-Leu-Ile-Cys]-NH₂ (GIP-326) of the following formula

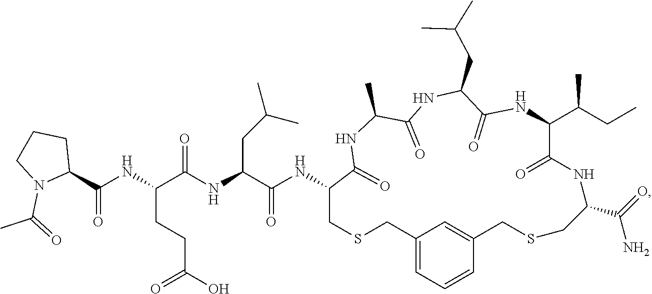 [SMILES: CC[C@H](C)[C@@H]1NC(=O)[C@H](CC(C)C)NC(=O)[C@H](C)NC(=O)[C@@H](NC(=O)[C@H](CC(C)C)NC(=O)[C@H](CCC(=O)O)NC(=O)[C@@H]2CCCN2C(C)=O)CSCc2cccc(c2)CSC[C@@H](C(N)=O)NC1=O]

compound Ac-Glu-Leu-[Cys(3MeBn)-Ala-Leu-Ile-Cys]-NH₂ (GIP-327) of the following formula

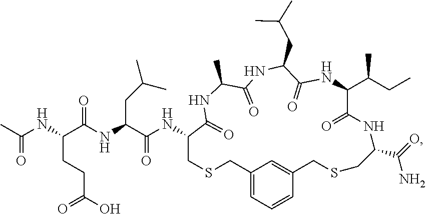 [SMILES: CC[C@H](C)[C@@H]1NC(=O)[C@H](CC(C)C)NC(=O)[C@H](C)NC(=O)[C@@H](NC(=O)[C@H](CC(C)C)NC(=O)[C@H](CCC(=O)O)NC(C)=O)CSCc2cccc(c2)CSC[C@@H](C(N)=O)NC1=O]

compound H-Met-Tyr-Asp-Trp-Trp-pro-Glu-Leu-[Cys(3MeBn)-Ala-Leu-Ile-Cys]-NH₂ (GIP-328) of the following formula

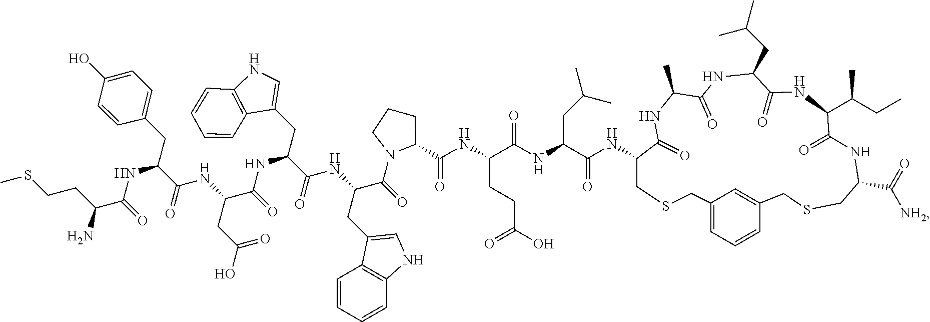

compound H-Met-Tyr-Asp-Trp-trp-Pro-Glu-Leu-[Cys(3MeBn)-Ala-Leu-Ile-Cys]-NH₂ (GIP-329) of the following formula

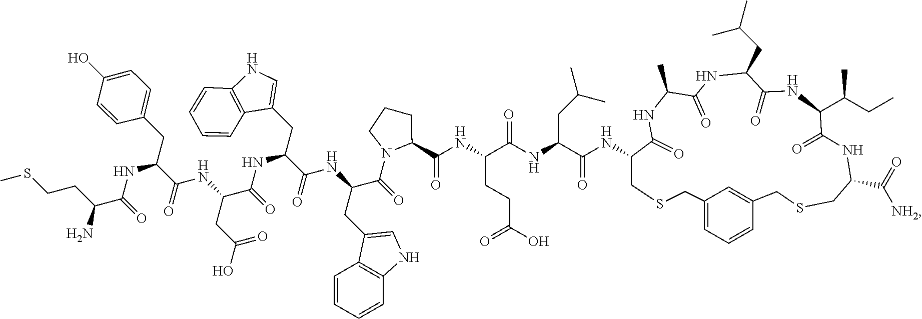 [SMILES: CC[C@H](C)[C@@H]1NC(=O)[C@H](CC(C)C)NC(=O)[C@H](C)NC(=O)[C@@H](NC(=O)[C@H](CC(C)C)NC(=O)[C@H](CCC(=O)O)NC(=O)[C@@H]2CCCN2C(=O)[C@@H](Cc2c[nH]c3ccccc23)NC(=O)[C@H](Cc2c[nH]c3ccccc23)NC(=O)[C@H](CC(=O)O)NC(=O)[C@H](Cc2ccc(O)cc2)NC(=O)[C@@H](N)CCSC)CSCc2cccc(c2)CSC[C@@H](C(N)=O)NC1=O]

compound H-Met-Tyr-Asp-Trp-Trp-Pro-Glu-Leu-[Cys(3MeBn)-Ala-leu-Ile-Cys]-NH₂ (GIP-330) of the following formula

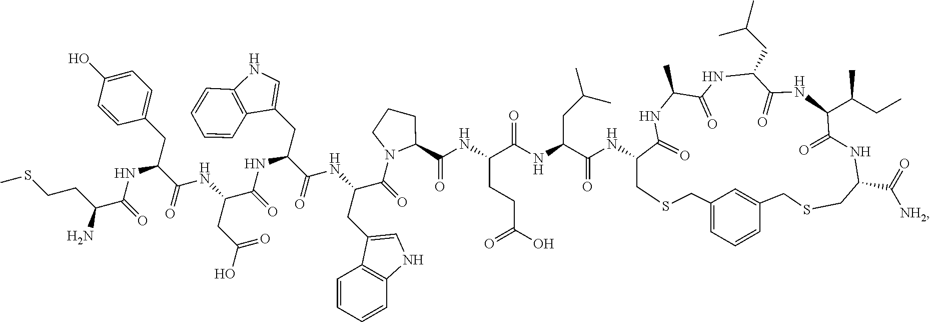

compound H-Met-Tyr-Asp-Trp-Trp-Pro-Glu-Leu-[Cys(4MeBn)-Ala-Leu-Ile-Cys]-NH₂ (GIP-331) of the following formula

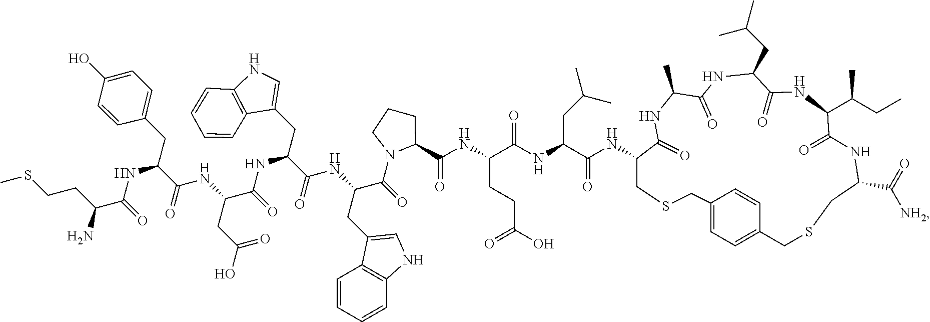

compound H-Met-Tyr-Ala-Trp-Trp-Pro-Glu-Leu-[Cys(3MeBn)-Ala-Leu-Ile-Cys]-NH₂ (GIP-332) of the following formula

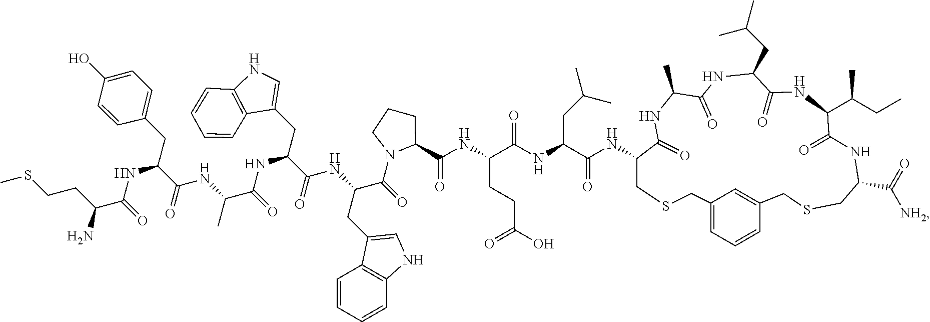

compound H-Met-Tyr-Asp-Ala-Trp-Pro-Glu-Leu-[Cys(3MeBn)-Ala-Leu-Ile-Cys]-NH₂ (GIP-333) of the following formula

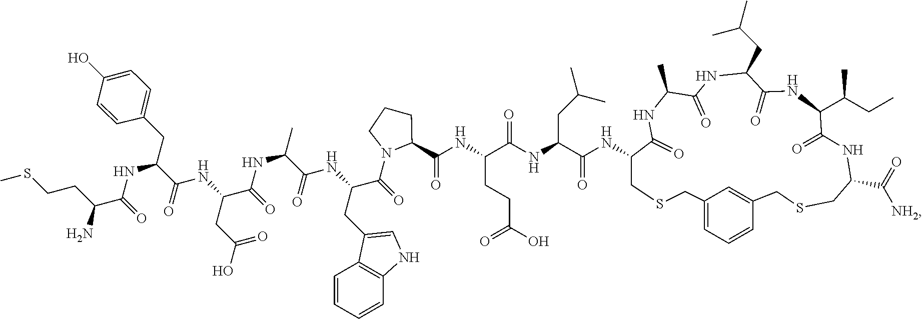 [SMILES: CC[C@H](C)[C@@H]1NC(=O)[C@H](CC(C)C)NC(=O)[C@H](C)NC(=O)[C@@H](NC(=O)[C@H](CC(C)C)NC(=O)[C@H](CCC(=O)O)NC(=O)[C@@H]2CCCN2C(=O)[C@H](Cc2c[nH]c3ccccc23)NC(=O)[C@H](C)NC(=O)[C@H](CC(=O)O)NC(=O)[C@H](Cc2ccc(O)cc2)NC(=O)[C@@H](N)CCSC)CSCc2cccc(c2)CSC[C@@H](C(N)=O)NC1=O]

compound H-Met-Tyr-Asp-Trp-Ala-Pro-Glu-Leu-[Cys(3MeBn)-Ala-Leu-Ile-Cys]-NH₂ (GIP-334) of the following formula

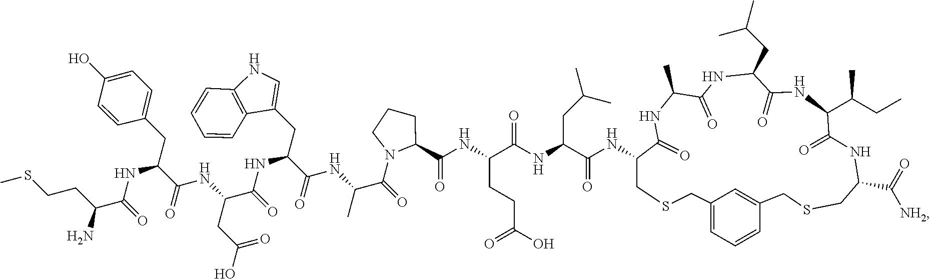

compound H-Met-Tyr-Asp-Trp-Trp-Pro-Ala-Leu-[Cys(3MeBn)-Ala-Leu-Ile-Cys]-NH₂ (GIP-335) of the following formula

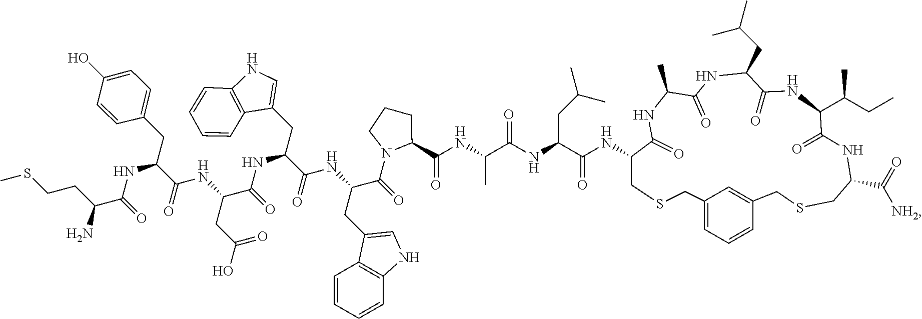 [SMILES: CC[C@H](C)[C@@H]1NC(=O)[C@H](CC(C)C)NC(=O)[C@H](C)NC(=O)[C@@H](NC(=O)[C@H](CC(C)C)NC(=O)[C@H](C)NC(=O)[C@@H]2CCCN2C(=O)[C@H](Cc2c[nH]c3ccccc23)NC(=O)[C@H](Cc2c[nH]c3ccccc23)NC(=O)[C@H](CC(=O)O)NC(=O)[C@H](Cc2ccc(O)cc2)NC(=O)[C@@H](N)CCSC)CSCc2cccc(c2)CSC[C@@H](C(N)=O)NC1=O]

compound H-Met-Tyr-Asp-Trp-Trp-Pro-Glu-Ala-[Cys(3MeBn)-Ala-Leu-Ile-Cys]-NH₂ (GIP-336) of the following formula

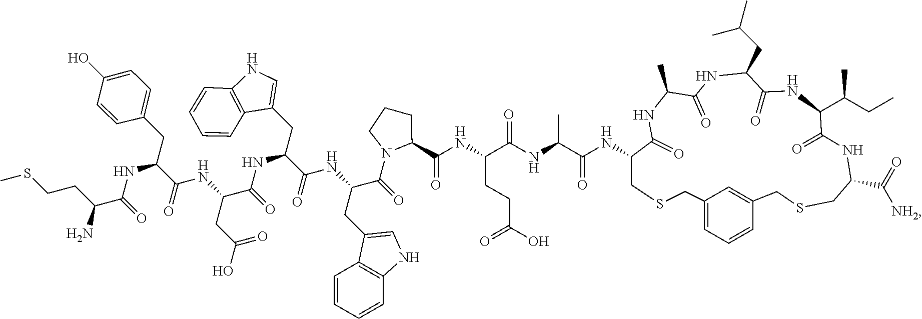 [SMILES: CC[C@H](C)[C@@H]1NC(=O)[C@H](CC(C)C)NC(=O)[C@H](C)NC(=O)[C@@H](NC(=O)[C@H](C)NC(=O)[C@H](CCC(=O)O)NC(=O)[C@@H]2CCCN2C(=O)[C@H](Cc2c[nH]c3ccccc23)NC(=O)[C@H](Cc2c[nH]c3ccccc23)NC(=O)[C@H](CC(=O)O)NC(=O)[C@H](Cc2ccc(O)cc2)NC(=O)[C@@H](N)CCSC)CSCc2cccc(c2)CSC[C@@H](C(N)=O)NC1=O]

compound H-Met-Tyr-Asp-Trp-Trp-Pro-Glu-Leu-[Cys(3MeBn)-Ala-Leu-Ala-Cys]-NH₂ (GIP-337) of the following formula

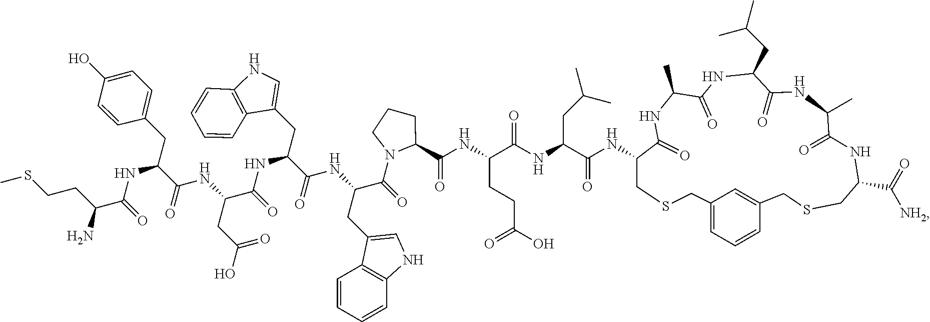

compound Ac-Asp-Trp-Trp-Pro-Glu-Leu-[Pen(3MeBn)-Aib-Leu-Ile-Cys]-NH₂ (GIP-338) of the following formula

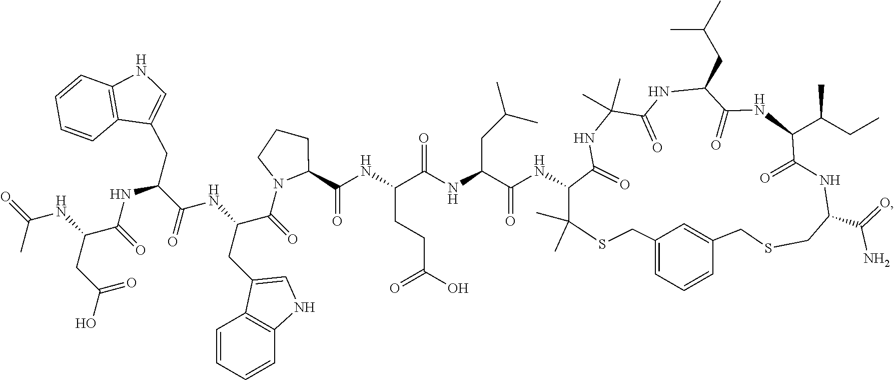 [SMILES: CC[C@H](C)[C@@H]1NC(=O)[C@H](CC(C)C)NC(=O)C(C)(C)NC(=O)[C@@H](NC(=O)[C@H](CC(C)C)NC(=O)[C@H](CCC(=O)O)NC(=O)[C@@H]2CCCN2C(=O)[C@H](Cc2c[nH]c3ccccc23)NC(=O)[C@H](Cc2c[nH]c3ccccc23)NC(=O)[C@H](CC(=O)O)NC(C)=O)C(C)(C)SCc2cccc(c2)CSC[C@@H](C(N)=O)NC1=O]

compound Ac-Asp-Trp-Trp-Pro-Glu-Leu-[Cys(3MeBn)-Aib-Leu-Ile-Pen]-NH₂ (GIP-339) of the following formula

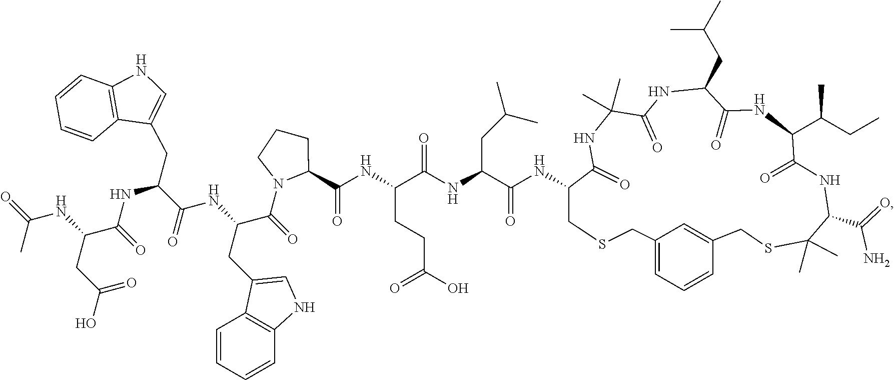 [SMILES: CC[C@H](C)[C@@H]1NC(=O)[C@H](CC(C)C)NC(=O)C(C)(C)NC(=O)[C@@H](NC(=O)[C@H](CC(C)C)NC(=O)[C@H](CCC(=O)O)NC(=O)[C@@H]2CCCN2C(=O)[C@H](Cc2c[nH]c3ccccc23)NC(=O)[C@H](Cc2c[nH]c3ccccc23)NC(=O)[C@H](CC(=O)O)NC(C)=O)CSCc2cccc(c2)CSC(C)(C)[C@@H](C(N)=O)NC1=O]

compound Ac-Asp-Trp-1Ni-Pro-Glu-Asn-[Cys(3MeBn)-Aib-Leu-Ile-Cys]-NH₂ (GIP-340) of the following formula

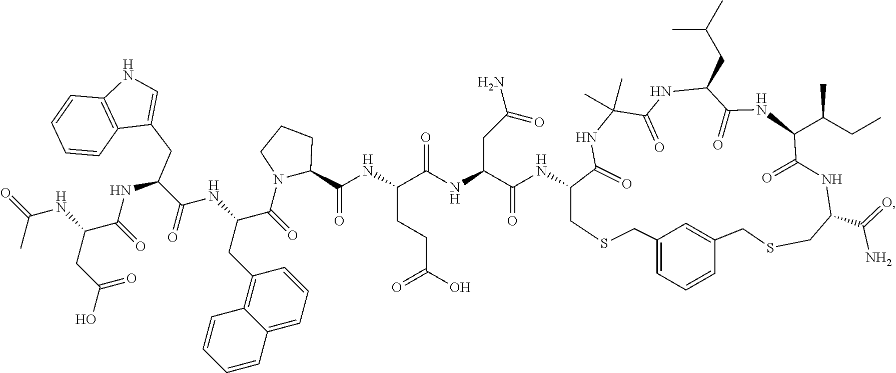 [SMILES: CC[C@H](C)[C@@H]1NC(=O)[C@H](CC(C)C)NC(=O)C(C)(C)NC(=O)[C@@H](NC(=O)[C@H](CC(N)=O)NC(=O)[C@H](CCC(=O)O)NC(=O)[C@@H]2CCCN2C(=O)[C@H](Cc2cccc3ccccc23)NC(=O)[C@H](Cc2c[nH]c3ccccc23)NC(=O)[C@H](CC(=O)O)NC(C)=O)CSCc2cccc(c2)CSC[C@@H](C(N)=O)NC1=O]

compound Ac-Asp-Trp-1Ni-4Dfp-Glu-Leu-[Cys(3MeBn)-Aib-Leu-Ile-Cys]-NH₂ (GIP-341) of the following formula

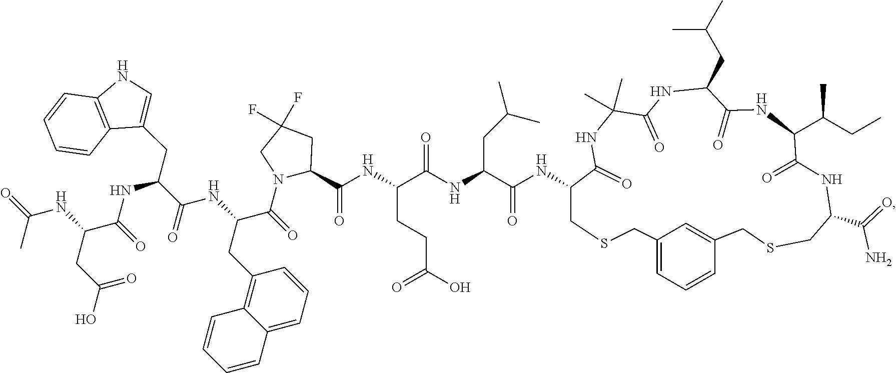 [SMILES: CC[C@H](C)[C@@H]1NC(=O)[C@H](CC(C)C)NC(=O)C(C)(C)NC(=O)[C@@H](NC(=O)[C@H](CC(C)C)NC(=O)[C@H](CCC(=O)O)NC(=O)[C@@H]2CC(F)(F)CN2C(=O)[C@H](Cc2cccc3ccccc23)NC(=O)[C@H](Cc2c[nH]c3ccccc23)NC(=O)[C@H](CC(=O)O)NC(C)=O)CSCc2cccc(c2)CSC[C@@H](C(N)=O)NC1=O]

compound Ac-Asp-Trp-1Ni-Rni-Glu-Leu-[Cys(3MeBn)-Aib-Leu-Ile-Cys]-NH₂ (GIP-342) of the following formula

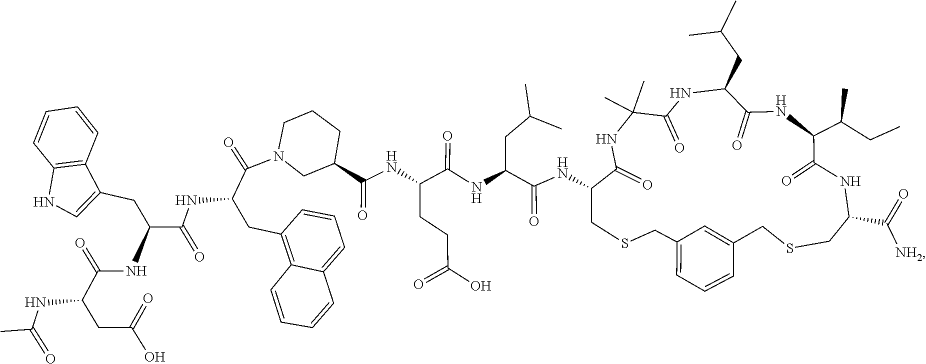 [SMILES: CC[C@H](C)[C@@H]1NC(=O)[C@H](CC(C)C)NC(=O)C(C)(C)NC(=O)[C@@H](NC(=O)[C@H](CC(C)C)NC(=O)[C@H](CCC(=O)O)NC(=O)[C@@H]2CCCN(C(=O)[C@H](Cc3cccc4ccccc34)NC(=O)[C@H](Cc3c[nH]c4ccccc34)NC(=O)[C@H](CC(=O)O)NC(C)=O)C2)CSCc2cccc(c2)CSC[C@@H](C(N)=O)NC1=O]

compound Ac-Asp-Trp-1Ni-Pro-Glu-Thi-[Cys(3MeBn)-Aib-Leu-Ile-Cys]-NH₂ (GIP-343) of the following formula

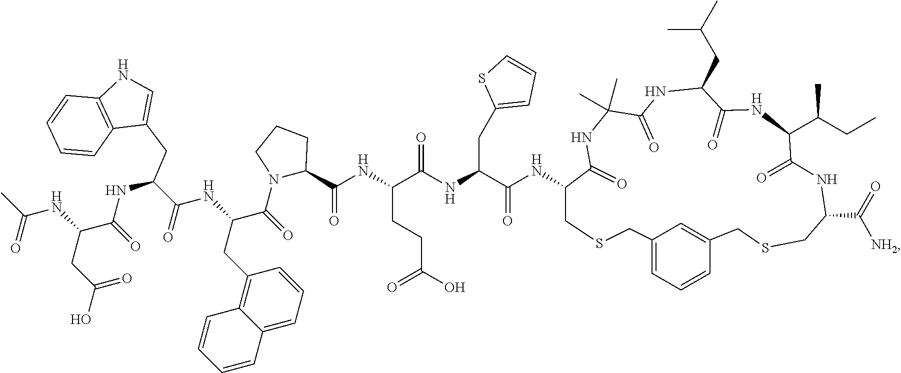 [SMILES: CC[C@H](C)[C@@H]1NC(=O)[C@H](CC(C)C)NC(=O)C(C)(C)NC(=O)[C@@H](NC(=O)[C@H](Cc2cccs2)NC(=O)[C@H](CCC(=O)O)NC(=O)[C@@H]2CCCN2C(=O)[C@H](Cc2cccc3ccccc23)NC(=O)[C@H](Cc2c[nH]c3ccccc23)NC(=O)[C@H](CC(=O)O)NC(C)=O)CSCc2cccc(c2)CSC[C@@H](C(N)=O)NC1=O]

compound Ac-Asp-Trp-1Ni-Pro-Glu-Phe-[Cys(3MeBn)-Aib-Leu-Ile-Cys]-NH₂ (GIP-344) of the following formula

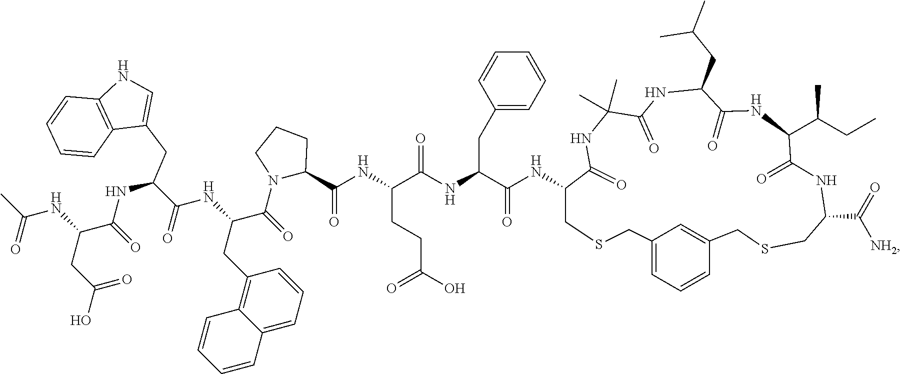 [SMILES: CC[C@H](C)[C@@H]1NC(=O)[C@H](CC(C)C)NC(=O)C(C)(C)NC(=O)[C@@H](NC(=O)[C@H](Cc2ccccc2)NC(=O)[C@H](CCC(=O)O)NC(=O)[C@@H]2CCCN2C(=O)[C@H](Cc2cccc3ccccc23)NC(=O)[C@H](Cc2c[nH]c3ccccc23)NC(=O)[C@H](CC(=O)O)NC(C)=O)CSCc2cccc(c2)CSC[C@@H](C(N)=O)NC1=O]

compound Ac-Asp-Trp-1Ni-Pro-Glu-Lys-[Cys(3MeBn)-Aib-Leu-Ile-Cys]-NH₂ (GIP-345) of the following formula

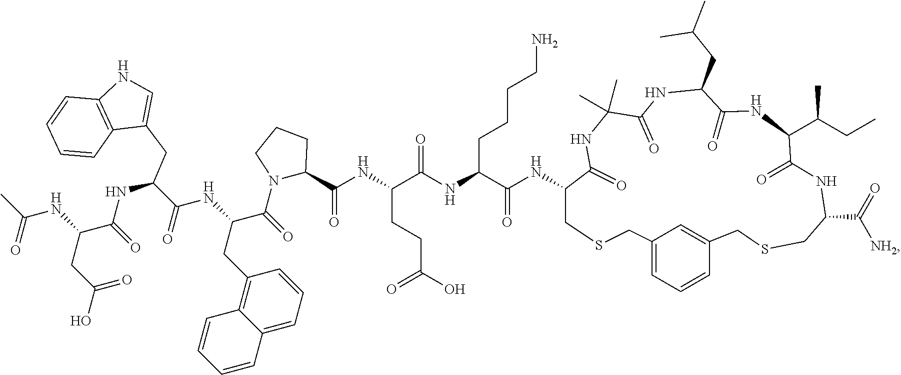 [SMILES: CC[C@H](C)[C@@H]1NC(=O)[C@H](CC(C)C)NC(=O)C(C)(C)NC(=O)[C@@H](NC(=O)[C@H](CCCCN)NC(=O)[C@H](CCC(=O)O)NC(=O)[C@@H]2CCCN2C(=O)[C@H](Cc2cccc3ccccc23)NC(=O)[C@H](Cc2c[nH]c3ccccc23)NC(=O)[C@H](CC(=O)O)NC(C)=O)CSCc2cccc(c2)CSC[C@@H](C(N)=O)NC1=O]

compound Ac-Asp-Trp-1Ni-Pro-Glu-Nle-[Cys(3MeBn)-Aib-Leu-Ile-Cys]-NH₂ (GIP-346) of the following formula

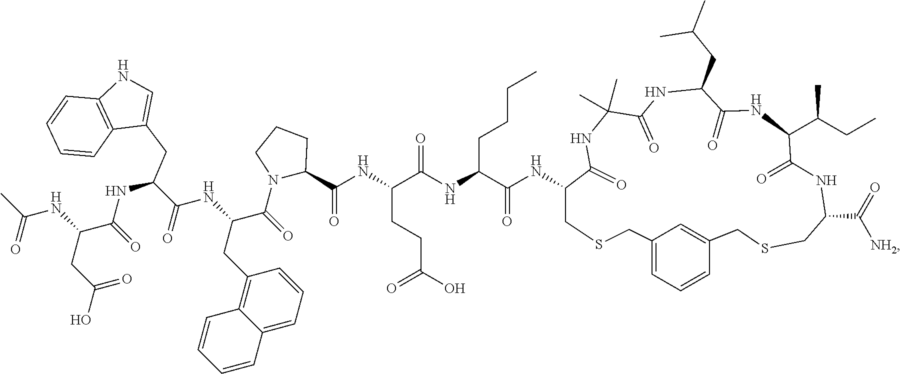 [SMILES: CCCC[C@H](NC(=O)[C@H](CCC(=O)O)NC(=O)[C@@H]1CCCN1C(=O)[C@H](Cc1cccc2ccccc12)NC(=O)[C@H](Cc1c[nH]c2ccccc12)NC(=O)[C@H](CC(=O)O)NC(C)=O)C(=O)N[C@H]1CSCc2cccc(c2)CSC[C@@H](C(N)=O)NC(=O)[C@H]([C@@H](C)CC)NC(=O)[C@H](CC(C)C)NC(=O)C(C)(C)NC1=O]

compound Ac-Asp-Trp-1Ni-Pro-Glu-Cit-[Cys(3MeBn)-Aib-Leu-Ile-Cys]-NH₂ (GIP-347) of the following formula

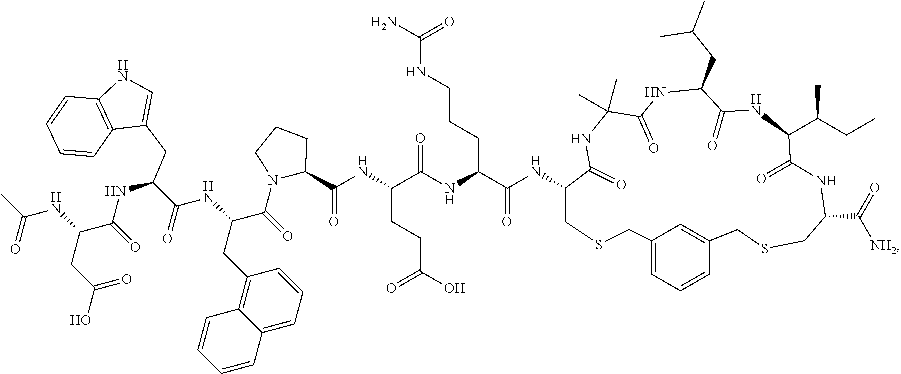 [SMILES: CC[C@H](C)[C@@H]1NC(=O)[C@H](CC(C)C)NC(=O)C(C)(C)NC(=O)[C@@H](NC(=O)[C@H](CCCNC(N)=O)NC(=O)[C@H](CCC(=O)O)NC(=O)[C@@H]2CCCN2C(=O)[C@H](Cc2cccc3ccccc23)NC(=O)[C@H](Cc2c[nH]c3ccccc23)NC(=O)[C@H](CC(=O)O)NC(C)=O)CSCc2cccc(c2)CSC[C@@H](C(N)=O)NC1=O]

compound Ac-Asp-Trp-1Ni-Pro-Glu-Dap(Ac)-[Cys(3MeBn)-Aib-Leu-Ile-Cys]-NH₂ (GIP-348) of the following formula

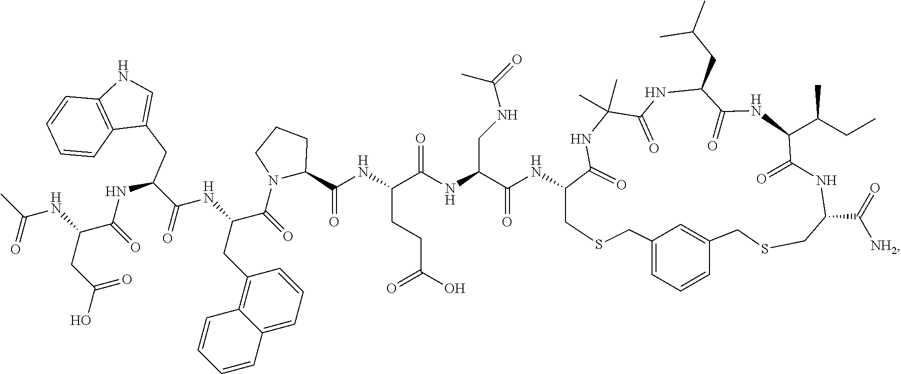 [SMILES: CC[C@H](C)[C@@H]1NC(=O)[C@H](CC(C)C)NC(=O)C(C)(C)NC(=O)[C@@H](NC(=O)[C@H](CNC(C)=O)NC(=O)[C@H](CCC(=O)O)NC(=O)[C@@H]2CCCN2C(=O)[C@H](Cc2cccc3ccccc23)NC(=O)[C@H](Cc2c[nH]c3ccccc23)NC(=O)[C@H](CC(=O)O)NC(C)=O)CSCc2cccc(c2)CSC[C@@H](C(N)=O)NC1=O]

compound Ac-Asp-Trp-Ocf-Hyp-Glu-leu-[Cys(2Lut)-Thp-Leu-Ile-Cys]-NH₂ (GIP-349) of the following formula

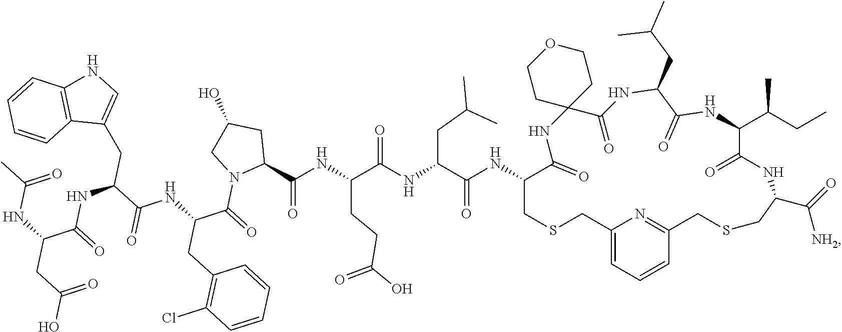 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2ccccc2Cl)NC(=O)[C@H](Cc2c[nH]c3ccccc23)NC(=O)[C@H](CC(=O)O)NC(C)=O)CSCc2cccc(n2)CSC[C@@H](C(N)=O)NC1=O]

compound Ac-Asp-Trp-Pcf-Hyp-Glu-leu-[Cys(2Lut)-Thp-Leu-Ile-Cys]-NH₂ (GIP-350) of the following formula

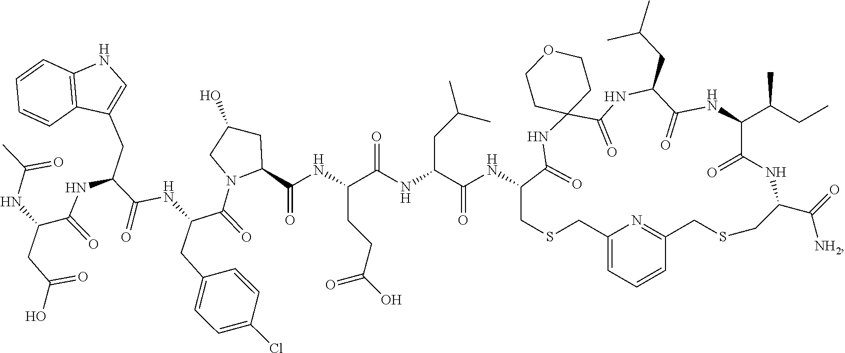 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2ccc(Cl)cc2)NC(=O)[C@H](Cc2c[nH]c3ccccc23)NC(=O)[C@H](CC(=O)O)NC(C)=O)CSCc2cccc(n2)CSC[C@@H](C(N)=O)NC1=O]

compound Ac-Asp-Trp-Egm-Hyp-Glu-leu-[Cys(2Lut)-Thp-Leu-Ile-Cys]-NH₂ (GIP-351) of the following formula

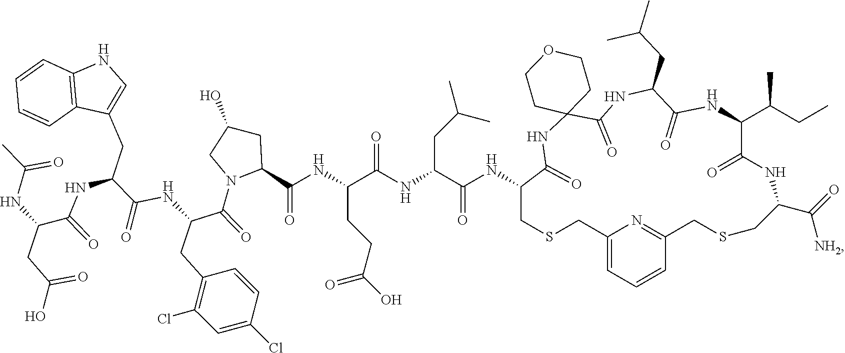 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2ccc(Cl)cc2Cl)NC(=O)[C@H](Cc2c[nH]c3ccccc23)NC(=O)[C@H](CC(=O)O)NC(C)=O)CSCc2cccc(n2)CSC[C@@H](C(N)=O)NC1=O]

compound Ac-Asp-Trp-Phe-Hyp-Glu-leu-[Cys(2Lut)-Thp-Leu-Ile-Cys]-NH₂ (GIP-352) of the following formula

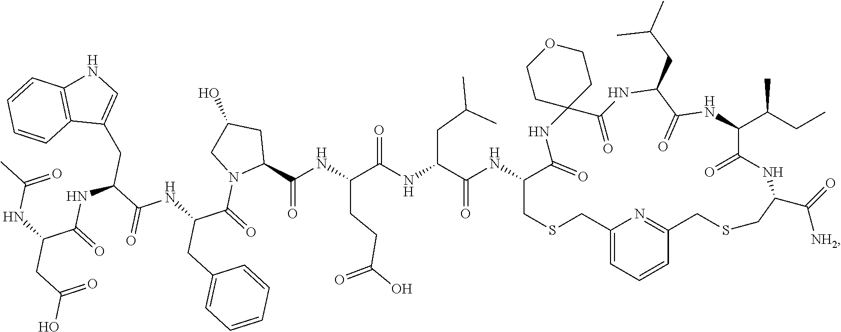 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2ccccc2)NC(=O)[C@H](Cc2c[nH]c3ccccc23)NC(=O)[C@H](CC(=O)O)NC(C)=O)CSCc2cccc(n2)CSC[C@@H](C(N)=O)NC1=O]

compound Ac-Asp-Trp-5Clw-Hyp-Glu-leu-[Cys(2Lut)-Thp-Leu-Arg-Cys]-NH₂ (GIP-353) of the following formula

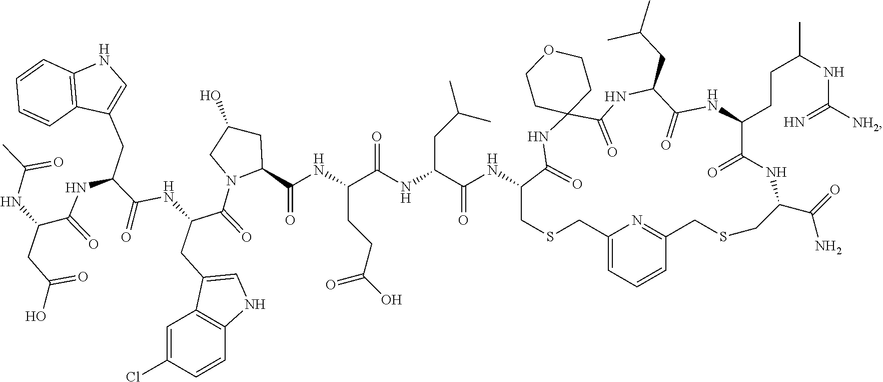 [SMILES: CC(=O)N[C@@H](CC(=O)O)C(=O)N[C@@H](Cc1c[nH]c2ccccc12)C(=O)N[C@@H](Cc1c[nH]c2ccc(Cl)cc12)C(=O)N1C[C@H](O)C[C@H]1C(=O)N[C@@H](CCC(=O)O)C(=O)N[C@H](CC(C)C)C(=O)N[C@H]1CSCc2cccc(n2)CSC[C@@H](C(N)=O)NC(=O)[C@H](CCC(C)NC(=N)N)NC(=O)[C@H](CC(C)C)NC(=O)C2(CCOCC2)NC1=O]

compound Ac-Asp-Trp-5Clw-Hyp-Glu-leu-[Cys(2Lut)-Thp-Leu-Glu-Cys]-NH₂ (GIP-354) of the following formula

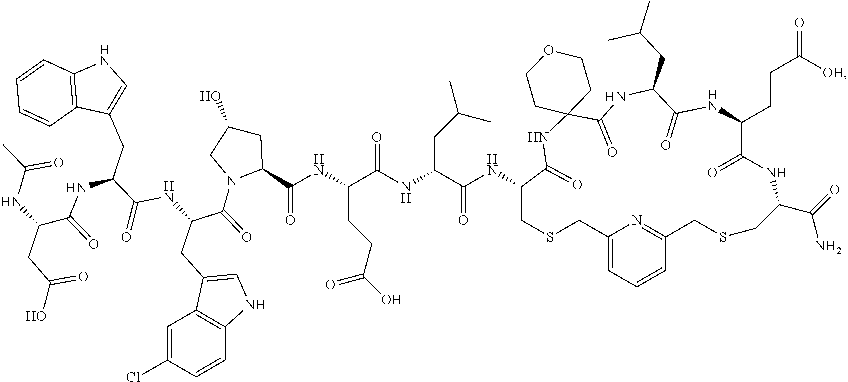 [SMILES: CC(=O)N[C@@H](CC(=O)O)C(=O)N[C@@H](Cc1c[nH]c2ccccc12)C(=O)N[C@@H](Cc1c[nH]c2ccc(Cl)cc12)C(=O)N1C[C@H](O)C[C@H]1C(=O)N[C@@H](CCC(=O)O)C(=O)N[C@H](CC(C)C)C(=O)N[C@H]1CSCc2cccc(n2)CSC[C@@H](C(N)=O)NC(=O)[C@H](CCC(=O)O)NC(=O)[C@H](CC(C)C)NC(=O)C2(CCOCC2)NC1=O]

compound Ac-Asp-Trp-5Clw-Hyp-Glu-leu-[Cys(2Lut)-Thp-Leu-Trp-Cys]-NH₂ (GIP-355) of the following formula

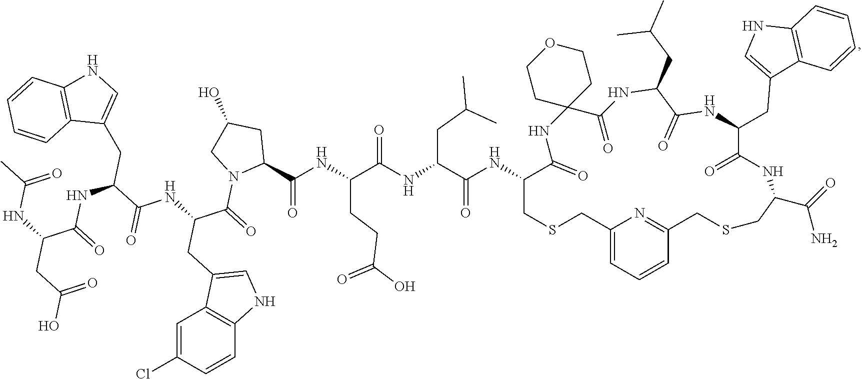 [SMILES: CC(=O)N[C@@H](CC(=O)O)C(=O)N[C@@H](Cc1c[nH]c2ccccc12)C(=O)N[C@@H](Cc1c[nH]c2ccc(Cl)cc12)C(=O)N1C[C@H](O)C[C@H]1C(=O)N[C@@H](CCC(=O)O)C(=O)N[C@H](CC(C)C)C(=O)N[C@H]1CSCc2cccc(n2)CSC[C@@H](C(N)=O)NC(=O)[C@H](Cc2c[nH]c3ccccc23)NC(=O)[C@H](CC(C)C)NC(=O)C2(CCOCC2)NC1=O]

compound Ac-Asp-Eaa-Eaa-Oic-Glu-leu-[Cys(2Lut)-Thp-Leu-Ile-Cys]-NH₂ (GIP-356) of the following formula

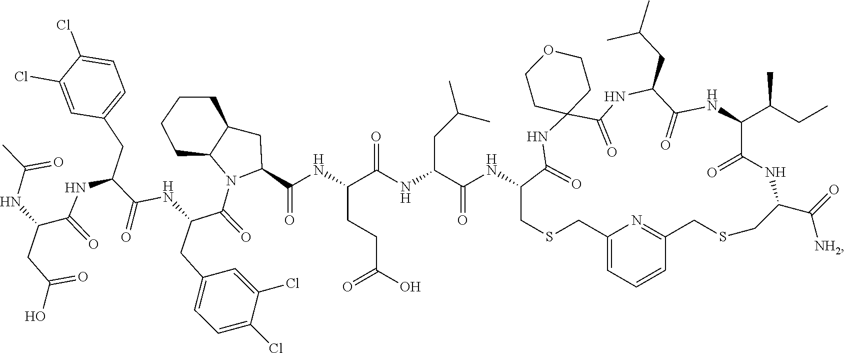 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H]3CCCC[C@@H]3N2C(=O)[C@H](Cc2ccc(Cl)c(Cl)c2)NC(=O)[C@H](Cc2ccc(Cl)c(Cl)c2)NC(=O)[C@H](CC(=O)O)NC(C)=O)CSCc2cccc(n2)CSC[C@@H](C(N)=O)NC1=O]

compound Ac-Asp-Eaa-Eaa-Nmg-Glu-leu-[Cys(2Lut)-Thp-Leu-Ile-Cys]-NH₂ (GIP-357) of the following formula

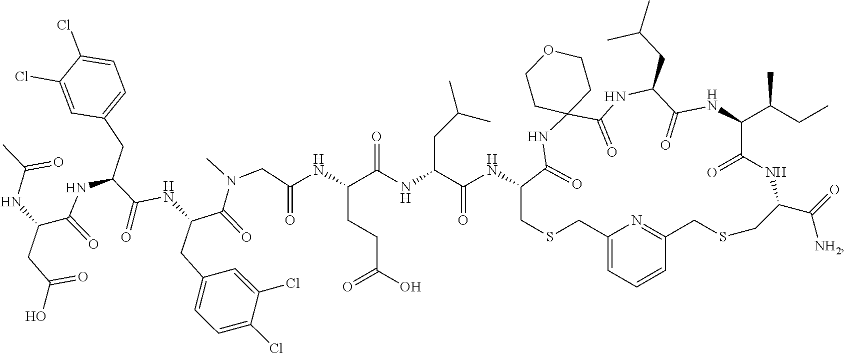 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)CN(C)C(=O)[C@H](Cc2ccc(Cl)c(Cl)c2)NC(=O)[C@H](Cc2ccc(Cl)c(Cl)c2)NC(=O)[C@H](CC(=O)O)NC(C)=O)CSCc2cccc(n2)CSC[C@@H](C(N)=O)NC1=O]

compound Ac-Asp-Trp-5Clw-Hyp-Glu-leu-[Cys-Thp-Leu-Ile-Cys]-NH₂ (GIP-364) of the following formula

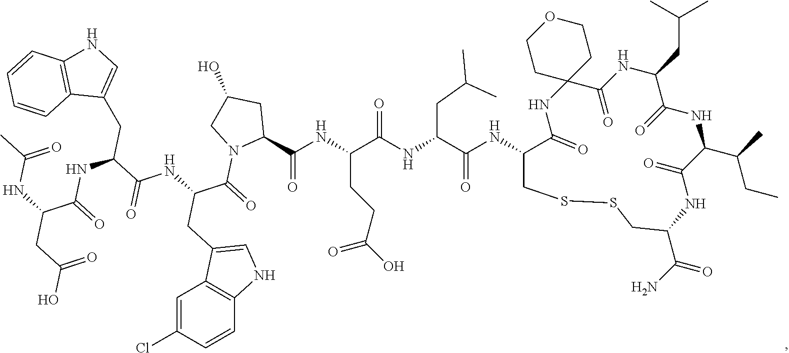 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CC(=O)O)NC(C)=O)CSSC[C@@H](C(N)=O)NC1=O]

compound H-Met-Tyr-Asp-Trp-Trp-Pro-Glu-Leu-Cys(Bzl)-Ala-Leu-Ile-Smc-NH₂ (GIP-368) of the following formula

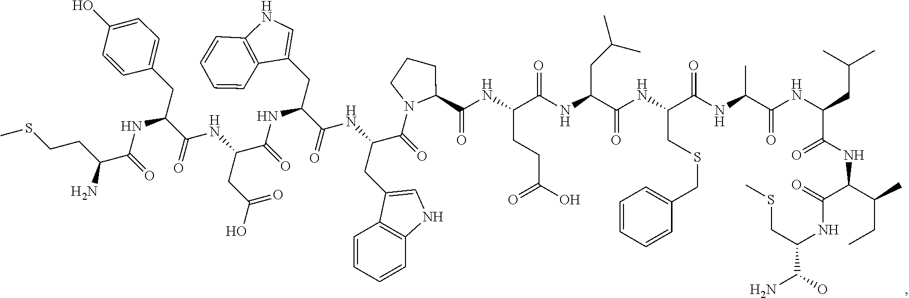 [SMILES: CC[C@H](C)[C@H](NC(=O)[C@H](CC(C)C)NC(=O)[C@H](C)NC(=O)[C@H](CSCc1ccccc1)NC(=O)[C@H](CC(C)C)NC(=O)[C@H](CCC(=O)O)NC(=O)[C@@H]1CCCN1C(=O)[C@H](Cc1c[nH]c2ccccc12)NC(=O)[C@H](Cc1c[nH]c2ccccc12)NC(=O)[C@H](CC(=O)O)NC(=O)[C@H](Cc1ccc(O)cc1)NC(=O)[C@@H](N)CCSC)C(=O)N[C@@H](CSC)[C@@H](N)O]

compound H-Met-Tyr-Asp-Trp-Trp-Pro-Glu-Leu-Smc-Ala-Leu-Ile-Cys(Bzl)-NH₂ (GIP-369) of the following formula

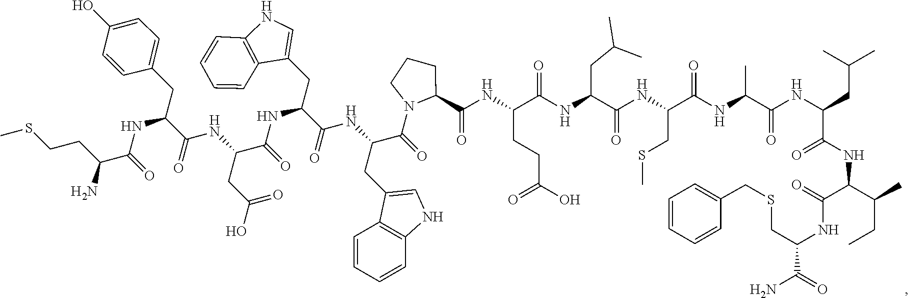 [SMILES: CC[C@H](C)[C@H](NC(=O)[C@H](CC(C)C)NC(=O)[C@H](C)NC(=O)[C@H](CSC)NC(=O)[C@H](CC(C)C)NC(=O)[C@H](CCC(=O)O)NC(=O)[C@@H]1CCCN1C(=O)[C@H](Cc1c[nH]c2ccccc12)NC(=O)[C@H](Cc1c[nH]c2ccccc12)NC(=O)[C@H](CC(=O)O)NC(=O)[C@H](Cc1ccc(O)cc1)NC(=O)[C@@H](N)CCSC)C(=O)N[C@@H](CSCc1ccccc1)C(N)=O]

compound DOTA-Ttds-Asp-Trp-Egn-Hyp-Glu-leu-[Cys(2Lut)-Thp-Leu-Ile-Cys]-NH₂ (GIP-401) or the following formula

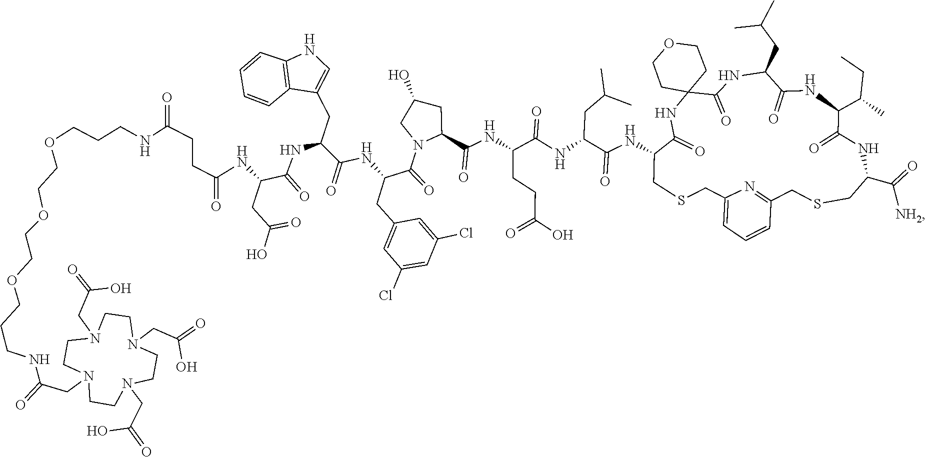 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2cc(Cl)cc(Cl)c2)NC(=O)[C@H](Cc2c[nH]c3ccccc23)NC(=O)[C@H](CC(=O)O)NC(=O)CCC(=O)NCCCOCCOCCOCCCNC(=O)CN2CCN(CC(=O)O)CCN(CC(=O)O)CCN(CC(=O)O)CC2)CSCc2cccc(n2)CSC[C@@H](C(N)=O)NC1=O]

compound Ac-Asp-Trp-Egn-Tap(DOTA)-Glu-leu-[Cys(2Lut)-Thp-Leu-Ile-Cys]-NH₂ (GIP-402) or the following formula

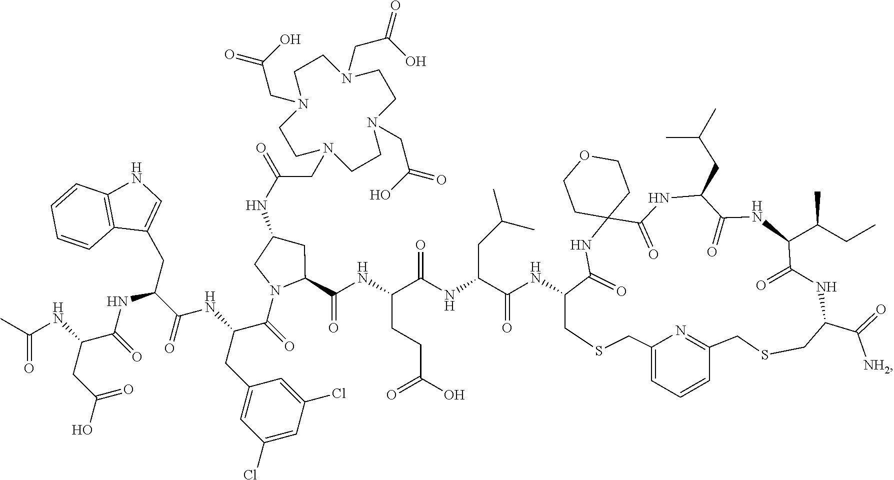 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](NC(=O)CN3CCN(CC(=O)O)CCN(CC(=O)O)CCN(CC(=O)O)CC3)CN2C(=O)[C@H](Cc2cc(Cl)cc(Cl)c2)NC(=O)[C@H](Cc2c[nH]c3ccccc23)NC(=O)[C@H](CC(=O)O)NC(C)=O)CSCc2cccc(n2)CSC[C@@H](C(N)=O)NC1=O]

compound Ac-Asp-Trp-Egn-Hyp-Glu-leu-[Cys(2Lut)-Apc(DOTA)-Leu-Ile-Cys]-NH₂ (GIP-403) or the following formula

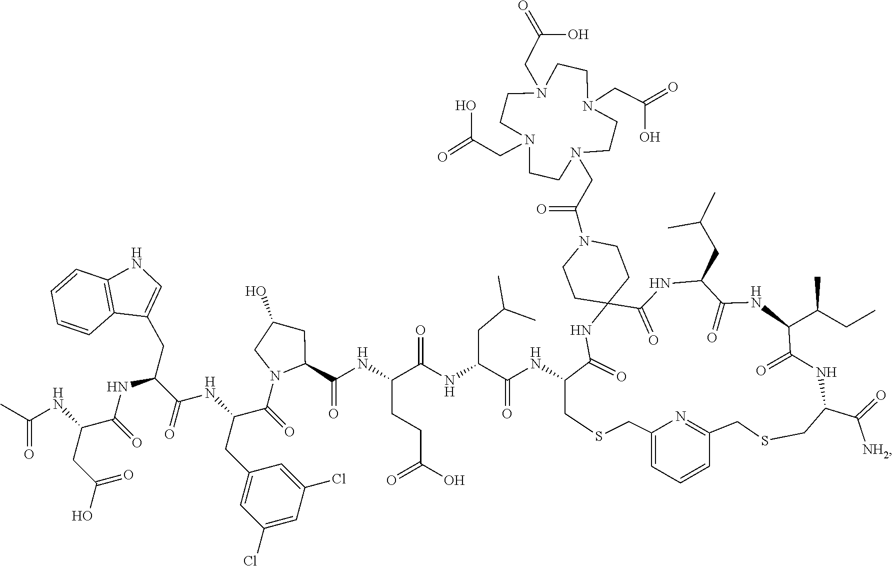 [SMILES: CC[C@H](C)[C@@H]1NC(=O)[C@H](CC(C)C)NC(=O)C2(CCN(C(=O)CN3CCN(CC(=O)O)CCN(CC(=O)O)CCN(CC(=O)O)CC3)CC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2cc(Cl)cc(Cl)c2)NC(=O)[C@H](Cc2c[nH]c3ccccc23)NC(=O)[C@H](CC(=O)O)NC(C)=O)CSCc2cccc(n2)CSC[C@@H](C(N)=O)NC1=O]

compound DOTA-Ttds-Asp-Trp-5Clw-Hyp-Glu-leu-[Cys(2Lut)-Thp-Leu-Ser-Cys]-NH₂ (GIP-404) or the following formula

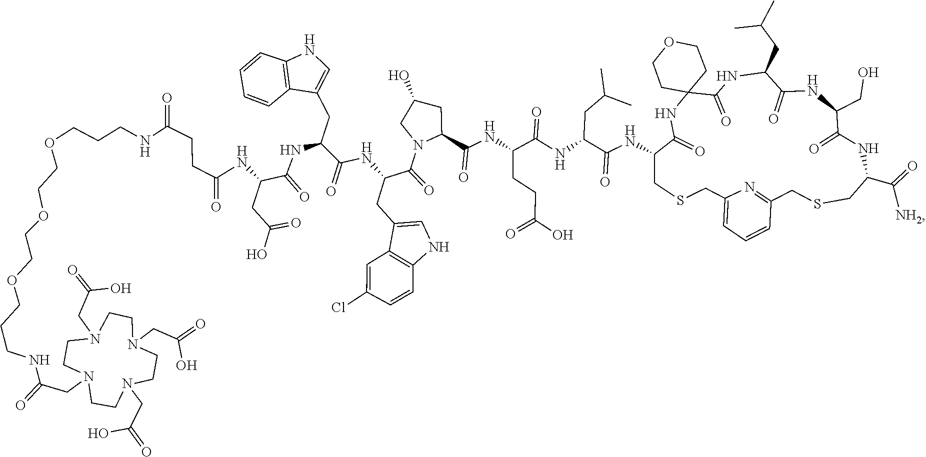 [SMILES: CC(C)C[C@@H]1NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CC(=O)O)NC(=O)CCC(=O)NCCCOCCOCCOCCCNC(=O)CN2CCN(CC(=O)O)CCN(CC(=O)O)CCN(CC(=O)O)CC2)CSCc2cccc(n2)CSC[C@@H](C(N)=O)NC(=O)[C@H](CO)NC1=O]

compound Ac-Asp-Trp-2Qi-Hyp-Glu-leu-[Cys(2Lut)-Thp-Leu-Ile-Cys]-NH₂ (GIP-405) or the following formula

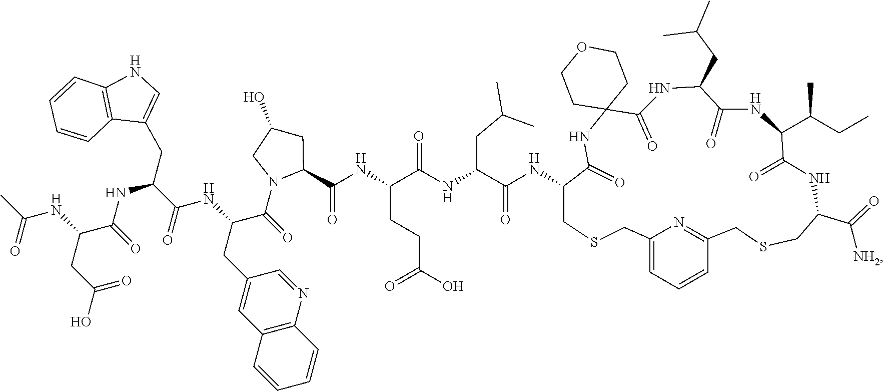 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2cnc3ccccc3c2)NC(=O)[C@H](Cc2c[nH]c3ccccc23)NC(=O)[C@H](CC(=O)O)NC(C)=O)CSCc2cccc(n2)CSC[C@@H](C(N)=O)NC1=O]

compound Ac-Asp-Trp-5Clw-Hyp-Glu-leu-[Cys(2Lut)-Thp-Leu-Ser-Cys]-lys(DOTA)-NH₂ (GIP-406) or the following formula

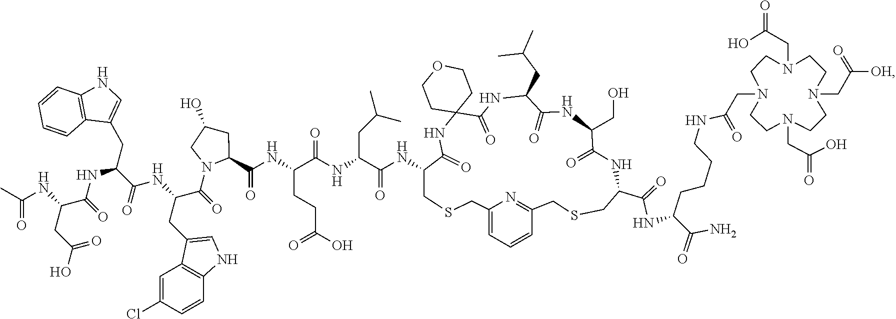 [SMILES: CC(=O)N[C@@H](CC(=O)O)C(=O)N[C@@H](Cc1c[nH]c2ccccc12)C(=O)N[C@@H](Cc1c[nH]c2ccc(Cl)cc12)C(=O)N1C[C@H](O)C[C@H]1C(=O)N[C@@H](CCC(=O)O)C(=O)N[C@H](CC(C)C)C(=O)N[C@H]1CSCc2cccc(n2)CSC[C@@H](C(=O)N[C@H](CCCCNC(=O)CN2CCN(CC(=O)O)CCN(CC(=O)O)CCN(CC(=O)O)CC2)C(N)=O)NC(=O)[C@H](CO)NC(=O)[C@H](CC(C)C)NC(=O)C2(CCOCC2)NC1=O]

compound Ac-Asp-Trp-5Clw-Hyp-Glu-leu-[Cys(2Lut)-Apc(DOTA)-Leu-Ser-Cys]-NH₂ (GIP-407) or the following formula

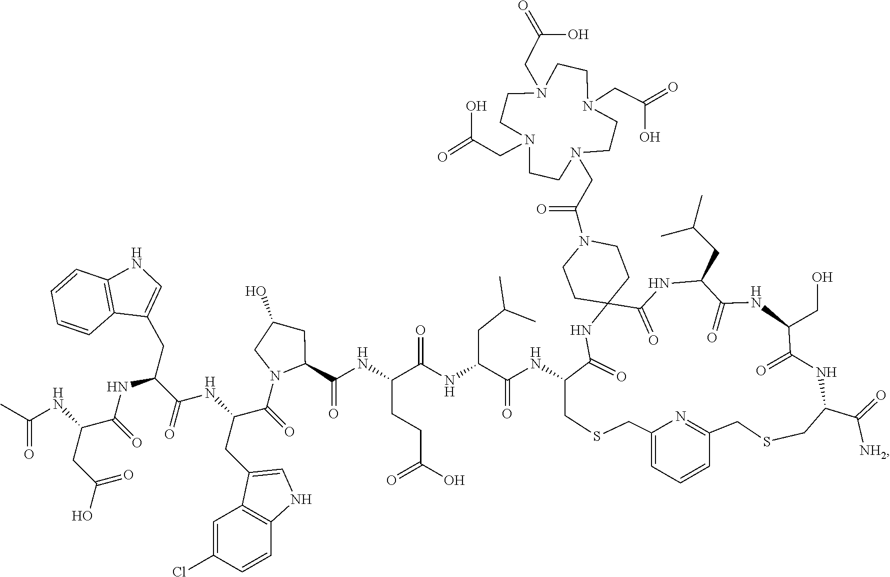 [SMILES: CC(=O)N[C@@H](CC(=O)O)C(=O)N[C@@H](Cc1c[nH]c2ccccc12)C(=O)N[C@@H](Cc1c[nH]c2ccc(Cl)cc12)C(=O)N1C[C@H](O)C[C@H]1C(=O)N[C@@H](CCC(=O)O)C(=O)N[C@H](CC(C)C)C(=O)N[C@H]1CSCc2cccc(n2)CSC[C@@H](C(N)=O)NC(=O)[C@H](CO)NC(=O)[C@H](CC(C)C)NC(=O)C2(CCN(C(=O)CN3CCN(CC(=O)O)CCN(CC(=O)O)CCN(CC(=O)O)CC3)CC2)NC1=O]

compound Ac-Asp-Trp-Egn-Hyp-Glu-leu-[Cys(2Lut)-Thp-Leu-Ile-Cys]-lys(DOTA)-NH₂ (GIP-408) or the following formula

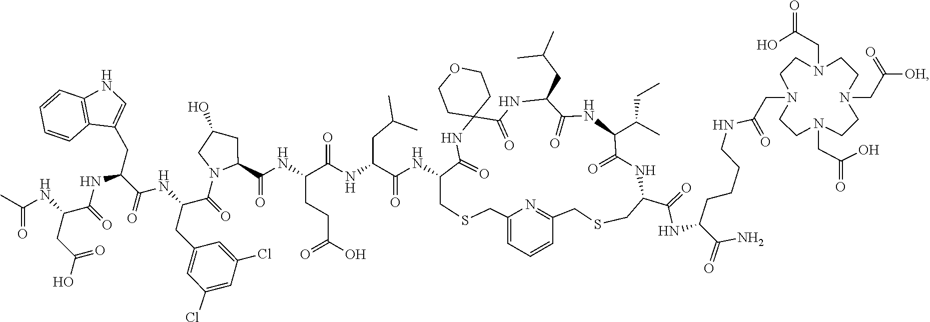 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2cc(Cl)cc(Cl)c2)NC(=O)[C@H](Cc2c[nH]c3ccccc23)NC(=O)[C@H](CC(=O)O)NC(C)=O)CSCc2cccc(n2)CSC[C@@H](C(=O)N[C@H](CCCCNC(=O)CN2CCN(CC(=O)O)CCN(CC(=O)O)CCN(CC(=O)O)CC2)C(N)=O)NC1=O]

and compound Ac-Asp-Trp-5Clw-Tap(DOTA)-Glu-leu-[Cys(2Lut)-Thp-Leu-Ser-Cys]-NH₂ (GIP-409) or the following formula

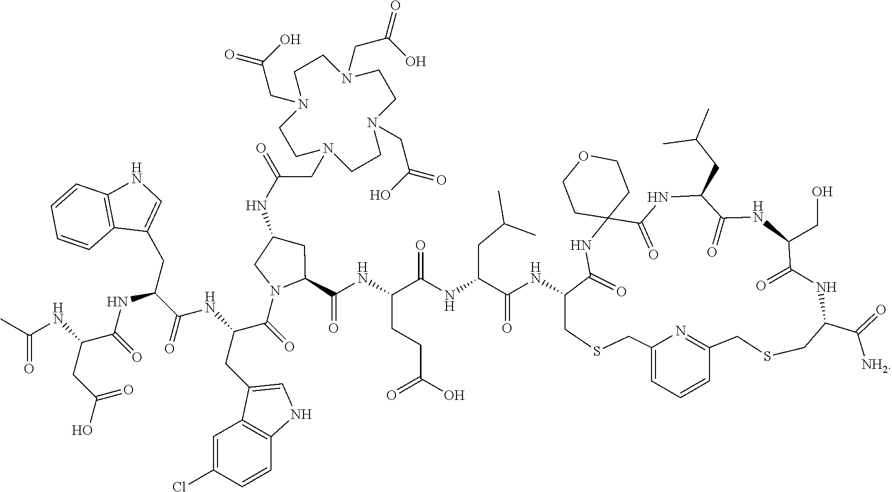 [SMILES: CC(=O)N[C@@H](CC(=O)O)C(=O)N[C@@H](Cc1c[nH]c2ccccc12)C(=O)N[C@@H](Cc1c[nH]c2ccc(Cl)cc12)C(=O)N1C[C@H](NC(=O)CN2CCN(CC(=O)O)CCN(CC(=O)O)CCN(CC(=O)O)CC2)C[C@H]1C(=O)N[C@@H](CCC(=O)O)C(=O)N[C@H](CC(C)C)C(=O)N[C@H]1CSCc2cccc(n2)CSC[C@@H](C(N)=O)NC(=O)[C@H](CO)NC(=O)[C@H](CC(C)C)NC(=O)C2(CCOCC2)NC1=O]

A general linear peptide is typically written from the N- to C-terminal direction as shown below:
A-Xaa1-Xaa2-Xaa3-Xaa4- . . . Xaan-C-term;

Therein
1. ‘Xaax’ is the abbreviation, descriptor or symbol for amino acids or building blocks at specific sequence position x as shown in Table 5,
2. ‘A’ is the N-terminal modification group, e.g., an abbreviation for a specific terminating carboxylic acid like ‘Ac’ for acetyl or other chemical group or structural formula of chemical groups linked to the N-terminal amino acid code (Xaa1) via a hyphen and
3. ‘C-term’ is the C-terminal group which is typically ‘OH’ or ‘NH₂’ (as terminal carboxylic acid or amide) or an abbreviation for a specific terminating amine linked to the C-terminal amino acid code (Xaan) via a hyphen.
   Branched Peptides with Side Chains Modified by Specific Building Blocks or Peptides

Linear, branched peptides are written from the N- to C-terminal direction as shown below:
1. A-Xaa1-Xaa2-Xaa3(NT-Xab1-Xab2- . . . Xabn)- . . . Xaan-C-term
   or
2. A-Xaa1-Xaa2-Xaa3(Xac1-Xac2- . . . Xacn-C-term)- . . . Xaan-C-term

Therein, the statements 1.-3. of the description of linear peptides for the specification of ‘Xaax’, ‘A’ and ‘C-term’ in the main chain of the branched peptide apply.

The position of a branch is specified by parentheses right next to a ‘Xaax’ abbreviation. Branches typically either occur at lysine (Lys) residues or 4-aminopiperidine-4-carboxylic acid (Apc) (or similar), which means that the branch is attached to side chain ε-amino function of the lysine or to the nitrogen atom within the piperidine ring of 4-aminopiperidine-4-carboxylic acid via an amide bond.

The content of the parentheses describes the sequence/structure of the peptide branch ‘A-Xab1-Xab2- . . . Xabn’. Herein
1. ‘Xabx’ is the abbreviation, descriptor or symbol for amino acids or building blocks at specific sequence position x of the branch as shown in Table 5, whereby the linear connection of individual Xabx is indicated by a hyphen,
2. ‘A’ is an N-terminal group, e.g. an abbreviation for a specific terminating carboxylic acid like ‘Ac’ for acetyl or other chemical group or structural formula of chemical groups linked to the N-terminal amino acid code (Xab1) via a hyphen and
3. the last building block of the branch ‘Xabn’, which connects the branch with the main chain by forming an amide bond with its own carboxyl function with the side chain amino function of this lysine or 4-aminopiperidine-4-carboxylic acid (or similar residue).

Furthermore the content of the parenthesis may be a chemical group (e.g. AGLU) attached to a specific hetero atom of the side chain of said building block/amino acid Xaax. For clarification such residues (e.g. glu(AGLU)) are comprised in Table 4.

### Cyclic Peptides

An exemplaric general cyclic peptide written from the N- to C-terminal direction is shown below:
A-Xaa1-[Xaa2-Xaa3-Xaa4- . . . Xaan]-C-term;

Therein the statements 1.-3. of the description of linear peptides for the specification of ‘Xaax’, ‘A’ and ‘C-term’ in the main chain of the cyclic peptide apply. The characteristics of the peptide cycle are specified by square brackets.
1. The opening square bracket indicates the building block at whose side chain the cycle is initiated and
2. the closing square bracket indicates the building block at whose side chain the cycle is terminated.

The chemical nature of the connection between these two residues is
a disulphide bond in case that those indicated residues/amino acids contain sulfhydryl moieties (e.g., Cys).

### Cyclic Peptides Containing an Additional Cyclization Element (Yc)

A general extended cyclic peptide written from the N- to C-terminal direction is shown below:
A-Xaa1-[Xaa2(Yc)-Xaa3-Xaa4- . . . Xaan]-C-term;

Therein the statements 1.-3. of the description of linear peptides for the specification of ‘Xaax’, ‘A’ and ‘C-term’ in the main chain of the cyclic peptide apply. In addition, ‘Yc’ is the cyclization element. As in case of cyclic peptides, the characteristics of the cycle are specified by square brackets which indicate the building block at whose side chain the cycle is initiated and the building block at whose side chain the cycle is terminated (statements 1, and 2. of cyclic peptides).

The abbreviation of the structure representing the Yc element of the extended cycle is the content of the parentheses left adjacent to the building block at whose side chain the cycle is initiated. The Yc element is covalently attached to the side chain of said building block. Furthermore, the Yc element is covalently attached to the side chain of the building block at whose side chain the cycle is terminated. The chemical nature of the linkages between either of these residues the Yc element depend on side chain functionality of both corresponding amino acids Xaan. The linkage is a thioether if the side chain of Xaan contains a sulfhydryl group (e.g., Cys).

As non-limiting example for a branched, cyclic peptide containing an additional cyclization element peptide the structure of
AGLU-Glutar-Asp-Trp-5Clw-Hyp-Glu-leu-[Cys(2Lut)-Apc(AGLU-Glutar)-Leu-Ile-Cys]-APAc-Lys(DOTA)-NH₂
   is depicted below.

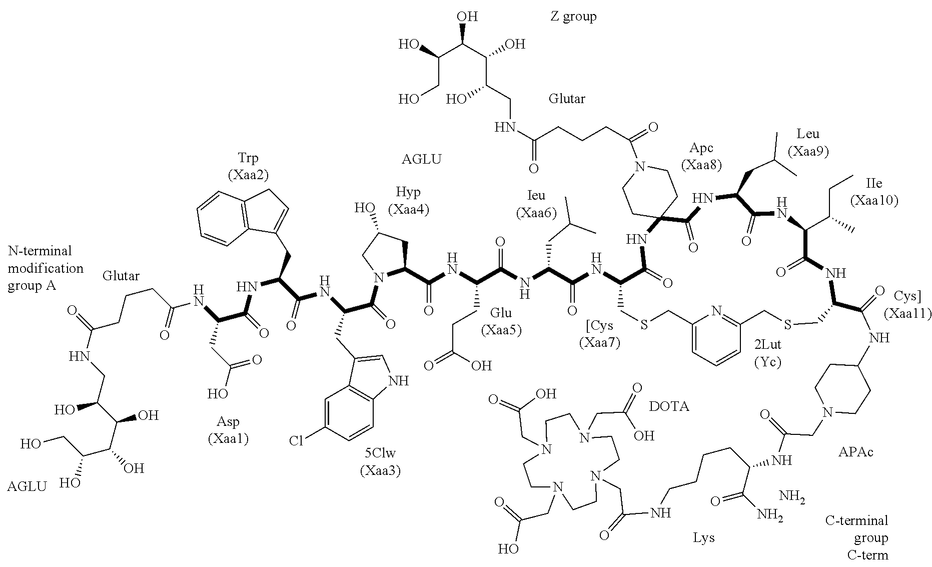

Therein
1. AGLU and Glutar bound to the N-terminal Asp (Xaa1) correspond to the ‘N-terminal modification group A’ in the general formula.
2. Asp, Trp, 5Clw, Hyp, Glu, leu, Cys, Apc, Leu, Ile, Cys correspond to ‘Xaa1’ to ‘Xaa11’ in the general formula. For clarity the main chain is represented with bold line thickness.
3. APAc, Lys, DOTA and NH₂ correspond to the ‘C-terminal group C-term’ in the general formula.
4. The opening square bracket (‘[’) adjacent to the N-terminal cysteine (Xaa7) in the sequence indicates that at this residue the cycle is initiated (cycle initiation residue).
5. The closing square bracket (‘]’) adjacent to the N-terminal cysteine (Xaa11) in the sequence indicates that at this residue the cycle is terminated (cycle termination residue).
6. 2Lut within the parentheses adjacent to the Cys indicated as initiation residue specifies the cyclization element ‘Yc’. It is further bound to the Cys indicated as cycle termination residue. The Yc element is connected to said residues via thioether linkages.
7. To the amino group of the Apc residue a Z group is attached. The latter consist of the bio-distribution modifier AGLU with the linker Glutar.

In accordance with the present invention, the compound of the present invention may comprise one or more of a Z group. The Z group comprises a bio-distribution modifier with an optional linker, a chelator with an optional linker or a combination thereof, wherein chelator and bio-distribution modifier with their respective optional linkers are linked by an amino acid residue. As preferably used, a linker is an element, moiety, or structure which separates two parts of a molecule. In the present invention, the linker forms a covalent bond with the chelator or the bio-distribution modifier or both and the respective part of the compounds of invention where Z is attached. A first functional group of the linker forming the bond with the chelator, the bio-distribution modifier or both of them may, in principle, be any chemical group which is capable of forming a bond, preferably a covalent bond with the chelator or the bio-distribution modifier group or both. In addition, the linker comprises a second functional group forming the bond, preferably a covalent bond with the part of the compounds of invention at the specified positions where Z is attached. Such second functional group may, in principle, be any chemical group which is capable of forming such bond with said part of the compound of invention at the specific positions where Z is attached.

An important property or feature of a linker is that it spaces apart the chelator or the bio-distribution modifier or both from the peptide part of the compound of invention. This is especially important in cases where the target binding ability of the peptide is compromised by the close proximity of the chelator. However, the overall linker length in its most extended conformer should not exceed 200 Å, preferably not more than 150 Å and most preferably not more than 100 Å.

A further important feature or property of a linker is that it enables the attachment of the chelator or the bio-distribution modifier or both to suitable positions of the peptide part of the compound of invention.

In an embodiment, a residue of a dicarboxylic acid represents a building block for a linker. Dicarboxylic acid-based linkers are used to enable the attachment of a chelator or a bio-distribution modifier to the N-terminal amine of the peptide of invention or an amino function of the side chain of an amino acid residue of the peptide of invention. Such N-terminal amine or amino side chain provides an amino function as conjugation group to which one of the at least two carboxy groups provided by the dicarboxylic acid building block is covalently attached forming an amide linkage. The bio-distribution modifier or chelator provides an amino function as conjugation group to which another of the at least two carboxy groups provided by the dicarboxylic acid building block is covalently attached forming an amide linkage, too. The very kind of dicarboxylic acid used as building block for a linker is dependent on the nature of the bio-distribution modifier or chelator and the attachment site on the peptide of invention and more specifically any functional group available at or provided by the bio-distribution modifier and chelator, respectively, which is reacted with one of the at least two carboxy groups of the dicarboxylic acid building block.

Preferred building blocks are dicarboxylic acids, which are selected from formulae (Lin1), (Lin2) or (Lin3)

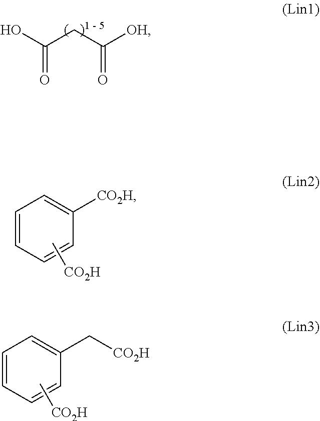

More preferred dicarboxylic acid building blocks such as formula (Lin1) are malonic acid, succinic acid, glutaric acid, adipic acid, wherein the two carboxylic acid groups are attached to ends of the aliphatic chain; such as formula (Lin2) phthalic acid, terephthalic acid, isophthalic acid and such as formula (Lin3) 2-, 3- or 4-carboxy-phenyl acetic acid. The most preferred dicarboxylic acid building block is glutaric acid (Glutar).

In an embodiment, a residue of a diamine represents a building block for a linker. Diamine based linkers are used to enable the attachment of a chelator or a bio-distribution modifier to the C-terminal carboxylic acid moiety of the peptide of invention or a carboxylic acid function of the side chain of an amino acid residue of the peptide of invention. Such C-terminal carboxylic acid or carboxylic acid side chain provides a carboxylic acid function as conjugation group to which one of the at least two amino groups provided by the diamine is covalently attached forming an amide linkage. The bio-distribution modifier or chelator provides a carboxylic acid as conjugation group to which another of the at least two amino groups provided by the diamine building block is covalently attached forming an amide linkage, too. The very kind of diamine used as building block for a linker is dependent on the nature of the bio-distribution modifier or chelator and the attachment site on the peptide of invention and more specifically any functional group available at or provided by the bio-distribution modifier and chelator, respectively, which is reacted with one of the at least two amino groups of the diamine building block.

Preferred building blocks are diamines, which are selected from formulae (Lin4), (Lin5), (Lin6) or (Lin7)

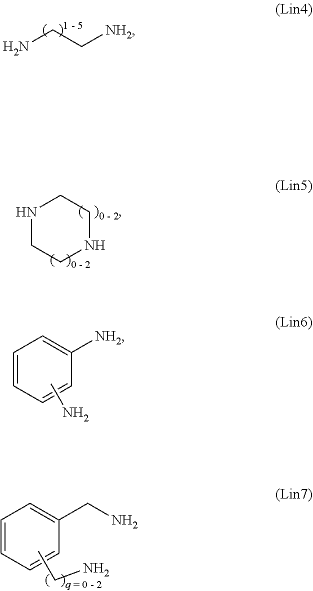

More preferred diamine building blocks are ethylenediamine (en), 1,4-diaminobutane, 1,5-diaminopentane (Ape), 1,6-diaminohexane, 1,3-dazetine, piperazine (PP), 1,3-diazinane, o-, m- and p-phenylenediamine, 4-aminobenzylamine, 1,4-bis(aminomethyl)benzene. The most preferred diamino building blocks are 1,5-diaminepentane (Ape), ethylenediamine (en) and piperazine (PP).

In an embodiment, a residue of an amino acid represents the building block for a linker. Amino acid-based linkers are used to enable the attachment of a chelator or a bio-distribution modifier to N-terminal amine, an amino function of the side chain of an amino acid residue or the C-terminal carboxylic acid moiety of the peptide of invention. Such N-terminal amine or amino side chain of the peptide of invention provides an amino function to which the carboxylic acid moiety of the amino acid linker is attached covalently by formation of an amide bond. The bio-distribution modifier or chelator provides a carboxylic acid as conjugation group, to which the amino function of the amino acid linker is attached covalently by formation of an amide bond. Furthermore, to the amino function of the linker, the carboxylic acid moiety of a further linker can be attached covalently by an amide bond, which preferably is an alpha amino acid linker. As an alternative such C-terminal carboxylic acid moiety of the peptide of invention provides a carboxylic acid group to which the amino function of the amino acid linker is attached covalently by formation of an amide bond. In this case the bio-distribution modifier or chelator provides an amine as conjugation group to which the carboxylic acid moiety of the linker is attached covalently by formation of an amide bond. Furthermore, to the carboxylic acid moiety of the linker, the amino function of a further linker can be attached covalently by an amide bond, which preferably is an alpha amino acid linker.

Preferred building blocks are amino acids, which are selected from formulae (Lin8), (Lin9), (Lin10), Lin(11) or Lin(12).

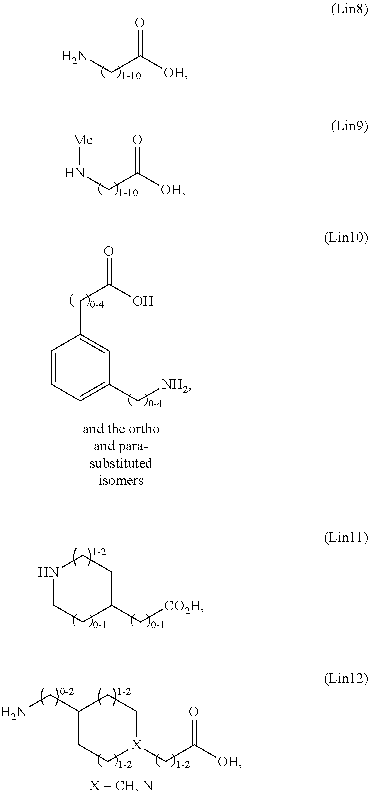

It is within the present invention that such amino acid linker is not further substituted. It is, however, also within the present invention that such amino acid linker is further substituted; preferably such substitution is CO—NH₂ and/or Ac—NH—.

Representatives of this kind of amino acid (structure Lin8), which can be used as a linker building block are glycine (Gly), beta-alanine (Bal), gamma-aminobutyric acid (GABA), 5-amino-pentanoic acid, 6-amino-hexanoic acid and homologs with up to 10 CH₂ groups.

Further representatives of this kind of amino acid (structure Lin9), which can be used as a linker building block are N-methylglycine (Nmg), N-methyl-beta-alanine, N-Methyl-gamma-aminobutyric acid, N-methyl-5-amino-pentanoic acid, N-methyl-6-amino-hexanoic acid and homologs with up to 10 CH₂ groups.

Further representatives of this kind of amino acid (structure Lin10), which can be used as a linker building block are 3-aminomethyl-benzoic acid, 4-aminomethyl-benzoic acid, anthranilic acid, 3-amino-benzoic acid and 4-amino-benzoic acid.

Further representatives of this kind of amino acid (structure Lin11), which can be used as a linker building block are 4-carboxymethyl-piperidine (Cmp), 3-piperidinecarboxylic acid (Nipecotic acid, Rni/Sni) and 4-piperidinecarboxylic acid (Isonipecotic acid, Inp).

Further representatives of this kind of amino acid (structure Lin12), which can be used as a linker building block are (4-aminopiperidin-1-yl)acetic acid (APAc), 2-(piperazin-1-yl)-acetic acid (PPAc) and 4-(aminomethyl)cyclohexanecarboxylic acid (4Amc).

It will be appreciated by a person skilled in the art that in case of amino acids with stereogenic centers all stereoisomeric forms may be used as such a linker.

In a further embodiment, the amino acid is an amino acid which contains, preferably as a backbone, a polyether. Preferably such polyether is polyethylene glycol and consists of up to 30 monomer units. Preferably, an amino acid comprising such polyether shows an increase in hydrophilicity compared to an amino acid not comprising such polyether. If incorporated as a linker the result is typically an increase in hydrophilicity. A preferred embodiment of this kind of amino acid is depicted in the following, wherein it will be acknowledged that such amino acid may comprise 0, 1, 2, 3, 4, 5, 6, 7, 8, 9, 10, 11 or 12 ethylene oxide moieties:

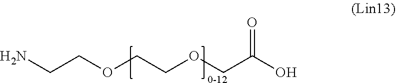

Preferred ethylene glycol containing amino acids are Ttds, O2Oc and PEG6.

In a further embodiment, a residue of an alpha amino acid represents a building block for a linker. Alpha amino acid-based linkers are used to enable the attachment of a chelator and/or a bio-distribution modifier to N-terminal amine, an amino function of the side chain of an amino acid residue of the peptide of invention or the C-terminal carboxylic acid moiety of the peptide of invention. Thereby simultaneous a chelator and a bio-distribution modifier or two bio-distribution modifiers can attached to one site of the peptide of the invention. Such N-terminal amine or amino side chain provides an amino function to which one of the one or more carboxy groups provided by the alpha amino acid building block is covalently attached forming an amide linkage. Such C-terminal carboxylic acid moiety provides a carboxylic acid function as conjugation group to which one of the one or more amino groups provided by the alpha amino acid covalently attached forming an amide linkage. In such cases wherein the alpha amino acid has more than one amino function the bio-distribution modifier or chelator provides a carboxylic acid moiety as conjugation group, which is attached covalently to said amino function of the alpha amino acid forming an amide linkage, while the other one of the amines is used as linkage to peptide of invention. In such cases wherein the alpha amino acid has more than one carboxylic acid moieties the bio-distribution modifier or chelator provides an amine as conjugation group, which is attached covalently to said carboxylic acid moiety of the alpha amino acid forming an amide linkage, while the other one of the carboxylic acid moieties is used as linkage to peptide of invention.

Preferred building blocks are alpha amino acids, which are selected from formula (Lin14) or (Lin15)

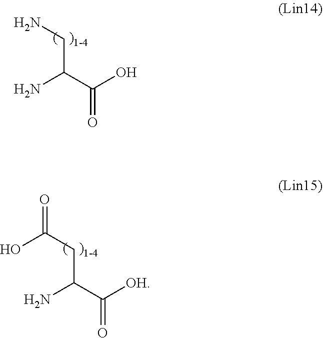

More preferred alpha amino acids building blocks are di-amino-propionic acid (Dap), di-amino-butyric acid (Dab), omithine, lysine (Lys), aspartic acid and glutamic acid. The most preferred alpha amino acid building blocks are lysine and glutamic acid. It will be appreciated by a person skilled in the art that in case of alpha amino acids with stereogenic centers all stereoisomeric forms may be used as such a linker.

However, the use of linkers usually follows a purpose. In some circumstances it is necessary to space a larger moiety apart from a bioactive molecule in order to retain high bioactivity. In other circumstances introduction of a linker opens the chance to tune physicochemical properties of the molecule by introduction of polarity or multiple charges. In certain circumstances the site of attachment within the sequence of the peptide of invention and the chemical nature of the bio-distribution modifier or chelator necessitate the implementation of a linker. Further, in certain circumstances it might be a strength and achievement if one can combine the chelator with a bioactive compound without the need for such linkers. Compounds comprising N-terminal bio-distribution modifiers typically comprise dicarboxylic acids for the linkage of the bio-distribution modifier to the bioactive compound. Compounds comprising C-terminal bio-distribution modifiers typically comprise alpha amino acids for the linkage of the bio-distribution modifier to the bioactive compound. Compounds of the present invention where the chelator is directly attached to the side chain of a residue of the bioactive compound under the formation of an amide bond typically perform excellently without the use of any dedicated linker.

It will be acknowledged by a person skilled in the art that the radioactive nuclide which is or which is to be attached to the compound of the invention, is selected taking into consideration the disease to be treated and/or the disease to be diagnosed, respectively, and/or the particularities of the patient and patient group, respectively, to be treated and to be diagnosed, respectively.

In an embodiment of the present invention, the radioactive nuclide is also referred to as radionuclide. Radioactive decay is the process by which an atomic nucleus of an unstable atom loses energy by emitting ionizing particles (ionizing radiation). There are different types of radioactive decay. A decay, or loss of energy, results when an atom with one type of nucleus, called the parent radionuclide, transforms to an atom with a nucleus in a different state, or to a different nucleus containing different numbers of protons and neutrons. Either of these products is named the daughter nuclide. In some decays the parent and daughter are different chemical elements, and thus the decay process results in nuclear transmutation (creation of an atom of a new element). For example, the radioactive decay can be alpha decay, beta decay, and gamma decay. Alpha decay occurs when the nucleus ejects an alpha particle (helium nucleus). This is the most common process of emitting nucleons, but in rarer types of decays, nuclei can eject protons, or specific nuclei of other elements (in the process called cluster decay). Beta decay occurs when the nucleus emits an electron (β⁻-decay) or positron (β⁺-decay) and a type of neutrino, in a process that changes a proton to a neutron or the other way around. By contrast, there exist radioactive decay processes that do not result in transmutation. The energy of an excited nucleus may be emitted as a gamma ray in gamma decay, or used to eject an orbital electron by interaction with the excited nucleus in a process called internal conversion, or used to absorb an inner atomic electron from the electron shell whereby the change of a nuclear proton to neutron causes the emission of an electron neutrino in a process called electron capture (EC), or may be emitted without changing its number of proton and neutrons in a process called isomeric transition (IT). Another form of radioactive decay, the spontaneous fission (SF), is found only in very heavy chemical elements resulting in a spontaneous breakdown into smaller nuclei and a few isolated nuclear particles.

In a preferred embodiment of the present invention, the radionuclide can be used for labeling of the compound of the invention.

In an embodiment of the present invention, the radionuclide is suitable for complexing with a chelator, leading to a radionuclide chelate complex.

In a further embodiment one or more atoms of the compound of the invention are of non-natural isotopic composition, preferably these atoms are radionuclides; more preferably radionuclides of carbon, oxygen, nitrogen, sulfur, phosphorus and halogens: These radioactive atoms are typically part of amino acids, in some case halogen containing amino acids, and/or building blocks and in some cases halogenated building blocks each of the compound of the invention.

In a preferred embodiment of the present invention, the radionuclide has a half-life that allows for diagnostic and/or therapeutic medical use. Specifically, the half-life is between 1 min and 100 days.

In a preferred embodiment of the present invention, the radionuclide has a decay energy that allows for diagnostic and/or therapeutic medical use. Specifically, for γ-emitting isotopes, the decay energy is between 0.01 and 4 MeV, preferably between 0.08 and 0.4 MeV, for diagnostic use. For positron-emitting isotopes, the mean decay energy is between 0.2 and 3 MeV, preferably between 0.2 and 1 MeV, for diagnostic use. For β⁻-emitting isotopes, the mean decay energy is between 0.02 and 3 MeV, preferably between 0.1 and 1 MeV, for therapeutic use. For α-emitting isotopes, the decay energy is between 3 and 8 MeV, preferably between 3.9 and 6.4 MeV, for therapeutic use.

In a preferred embodiment of the present invention, the radionuclide is industrially produced for medical use. Specifically, the radionuclide is available in GMP quality.

In a preferred embodiment of the present invention, the daughter nuclide(s) after radioactive decay of the radionuclide are compatible with the diagnostic and/or therapeutic medical use. Furthermore, the daughter nuclides are either stable or further decay in a way that does not interfere with or even support the diagnostic and/or therapeutic medical use. Representative radionuclides which may be used in connection with the present invention are summarized in Table 7.

TABLE 7
Key properties of preferred radionuclides - half
life, decay radiation types, decay energies of
transitions with highest probabilities (mean energies
for β decays) and absolute intensities (source:
https://www-nds.iaea.org/relnsd/vcharthtml/VChartHTML.html;
accessed Sep. 4^{th} 2020).

| Atomic | Radio- | | | | Energy | Intensity |
|---|---|---|---|---|---|---|
| number | nuclide | Half-life | | Decay | in MeV | (abs) in % |
| 6 | C-11 | 20.4 | m | β+ | 0.386 | 99.8 |
| 7 | N-13 | 10.0 | m | β+ | 0.492 | 99.8 |
| 9 | F-18 | 109.8 | m | β+ | 0.250 | 96.7 |
| 11 | Na-24 | 15.0 | h | β− | 0.554 | 99.9 |
| | | | | γ | 1.369 | 100.0 |
| | | | | γ | 2.754 | 99.9 |
| 12 | Mg-28 | 20.9 | h | β− | 0.156 | 94.8 |
| | | | | γ | 0.031 | 89.0 |
| | | | | γ | 1.342 | 54.0 |
| | | | | γ | 0.942 | 36.3 |
| | | | | γ | 0.401 | 35.9 |
| 14 | Si-31 | 157.4 | m | β− | 0.596 | 99.9 |
| 15 | P-32 | 14.3 | d | β− | 0.695 | 100.0 |
| 15 | P-33 | 25.4 | d | β− | 0.076 | 100.0 |
| 16 | S-38 | 170.3 | m | β− | 0.373 | 86.6 |
| | | | | γ | 1.942 | 86.5 |
| 17 | Cl-34m1 | 32.0 | m | β+ | 1.099 | 28.4 |
| | | | | β+ | 0.554 | 25.6 |
| | | | | γ | 2.127 | 42.8 |
| | | | | γ | 0.146 | 38.3 |
| | | | | γ | 1.177 | 14.1 |
| | | | | γ | 3.304 | 12.3 |
| 17 | Cl-38 | 37.2 | m | β− | 2.244 | 56.0 |
| | | | | β− | 0.420 | 32.9 |
| | | | | β− | 1.181 | 11.1 |
| | | | | γ | 2.167 | 44.0 |
| | | | | γ | 1.643 | 32.9 |
| 17 | Cl-39 | 56.2 | m | β− | 0.792 | 83.0 |
| | | | | γ | 1.267 | 53.6 |
| | | | | γ | 0.250 | 46.1 |
| | | | | γ | 1.517 | 39.3 |
| 18 | Ar-37 | 35.0 | d | EC | 0.814 | 100.0 |
| 18 | Ar-41 | 109.6 | m | β− | 0.459 | 99.2 |
| | | | | γ | 1.294 | 99.2 |
| 18 | Ar-44 | 11.9 | m | β− | 0.470 | 93.0 |
| | | | | β− | 1.369 | 12.0 |
| | | | | β− | 1.264 | 10.0 |
| | | | | γ | 0.183 | 66.0 |
| | | | | γ | 1.703 | 56.0 |
| | | | | γ | 1.886 | 31.0 |
| 9 | K-42 | 12.4 | h | β− | 1.566 | 81.9 |
| | | | | β− | 0.824 | 17.6 |
| | | | | γ | 1.525 | 18.1 |
| 19 | K-43 | 22.3 | h | β− | 0.305 | 90.9 |
| | | | | γ | 0.373 | 86.8 |
| | | | | γ | 0.617 | 79.2 |
| | | | | γ | 0.397 | 11.9 |
| | | | | γ | 0.593 | 11.3 |
| 19 | K-44 | 22.1 | m | β− | 2.601 | 34.0 |
| | | | | β− | 0.990 | 28.5 |
| | | | | β− | 0.816 | 11.0 |
| | | | | γ | 1.157 | 58.0 |
| | | | | γ | 2.151 | 23.0 |
| 19 | K-45 | 17.8 | m | β− | 0.973 | 51.0 |
| | | | | β− | 0.751 | 15.5 |
| | | | | β− | 0.733 | 10.0 |
| | | | | γ | 0.174 | 74.0 |
| | | | | γ | 1.706 | 53.0 |
| | | | | γ | 2.354 | 14.1 |
| 20 | Ca-47 | 4.5 | d | β− | 0.243 | 73.0 |
| | | | | β− | 0.819 | 27.0 |
| | | | | γ | 1.297 | 67.0 |
| 21 | Sc-43 | 3.9 | h | β+ | 0.508 | 70.9 |
| | | | | β+ | 0.344 | 17.2 |
| | | | | γ | 0.373 | 22.5 |
| 21 | Sc-44 | 4.0 | h | β+ | 0.632 | 94.3 |
| | | | | γ | 1.157 | 99.9 |
| 21 | Sc-44m1 | 58.6 | h | γ | 0.271 | 86.7 |
| 21 | Sc-47 | 3.3 | d | β− | 0.143 | 68.4 |
| | | | | β− | 0.204 | 31.6 |
| | | | | γ | 0.159 | 68.3 |
| 21 | Sc-48 | 43.7 | h | β− | 0.227 | 90.0 |
| | | | | β− | 0.159 | 10.0 |
| | | | | γ | 0.984 | 100.1 |
| | | | | γ | 1.312 | 100.1 |
| | | | | γ | 1.038 | 97.6 |
| 21 | Sc-49 | 57.2 | m | β− | 0.824 | 99.9 |
| 22 | Ti-45 | 184.8 | m | β+ | 0.439 | 84.8 |
| | | | | EC | 2.066 | 14.9 |
| 23 | V-47 | 32.6 | m | β+ | 0.832 | 96.5 |
| 23 | V-48 | 16.0 | d | β+ | 0.290 | 49.9 |
| | | | | EC | 1.717 | 39.2 |
| | | | | γ | 0.984 | 100.0 |
| | | | | γ | 1.312 | 98.2 |
| 24 | Cr-48 | 21.6 | h | EC | 1.235 | 96.1 |
| | | | | γ | 0.308 | 100.0 |
| | | | | γ | 0.112 | 96.0 |
| 24 | Cr-49 | 42.3 | m | β+ | 0.625 | 46.4 |
| | | | | β+ | 0.653 | 34.7 |
| | | | | β+ | 0.694 | 11.7 |
| | | | | γ | 0.091 | 53.2 |
| | | | | γ | 0.153 | 30.3 |
| | | | | γ | 0.062 | 16.4 |
| 24 | Cr-51 | 27.7 | d | EC | 0.752 | 90.1 |
| 25 | Mn-51 | 46.2 | m | β− | 0.964 | 96.9 |
| 25 | Mn-52 | 5.6 | d | β+ | 0.242 | 29.4 |
| | | | | EC | 1.597 | 61.4 |
| | | | | γ | 1.434 | 100.0 |
| | | | | γ | 0.936 | 94.5 |
| | | | | γ | 0.744 | 90.0 |
| 25 | Mn-52m1 | 21.1 | m | β+ | 1.174 | 96.4 |
| | | | | γ | 1.434 | 98.2 |
| 25 | Mn-56 | 2.6 | h | β− | 1.217 | 56.6 |
| | | | | β− | 0.382 | 27.5 |
| | | | | β− | 0.255 | 14.5 |
| | | | | γ | 0.847 | 98.9 |
| | | | | γ | 1.811 | 26.9 |
| | | | | γ | 2.113 | 14.2 |
| 26 | Fe-52 | 8.3 | h | β+ | 0.340 | 55.5 |
| | | | | EC | 1.825 | 43.6 |
| | | | | γ | 0.169 | 99.0 |
| 26 | Fe-59 | 44.5 | d | β− | 0.149 | 53.1 |
| | | | | β− | 0.081 | 45.3 |
| | | | | γ | 1.099 | 56.5 |
| | | | | γ | 1.292 | 43.2 |
| 27 | Co-55 | 17.5 | h | β+ | 0.649 | 46.0 |
| | | | | β+ | 0.436 | 25.6 |
| | | | | EC | 2.059 | 10.7 |
| | | | | γ | 0.931 | 75.0 |
| | | | | γ | 0.477 | 20.2 |
| | | | | γ | 1.409 | 16.9 |
| 27 | Co-61 | 1.6 | h | β− | 0.475 | 95.6 |
| | | | | γ | 0.067 | 84.7 |
| 27 | Co-62m1 | 13.9 | m | β− | 1.286 | 64.0 |
| | | | | β− | 0.888 | 19.6 |
| | | | | β− | 0.486 | 10.2 |
| | | | | γ | 1.173 | 97.7 |
| | | | | γ | 1.164 | 70.5 |
| | | | | γ | 2.004 | 18.2 |
| 28 | Ni-56 | 6.1 | d | EC | 0.416 | 100.0 |
| | | | | γ | 0.158 | 98.8 |
| | | | | γ | 0.812 | 86.0 |
| | | | | γ | 0.750 | 49.5 |
| | | | | γ | 0.270 | 36.5 |
| | | | | γ | 0.480 | 36.5 |
| | | | | γ | 1.562 | 14.0 |
| 28 | Ni-57 | 35.6 | h | β+ | 0.369 | 35.3 |
| | | | | EC | 1.871 | 29.2 |
| | | | | EC | 1.345 | 11.9 |
| | | | | EC | 1.734 | 10.0 |
| | | | | γ | 1.378 | 81.7 |
| | | | | γ | 0.127 | 16.7 |
| | | | | γ | 1.920 | 12.3 |
| 28 | Ni-65 | 2.5 | h | β− | 0.875 | 60.0 |
| | | | | β− | 0.221 | 28.4 |
| | | | | β− | 0.372 | 10.2 |
| | | | | γ | 1.482 | 23.6 |
| | | | | γ | 1.116 | 15.4 |
| 28 | Ni-66 | 54.6 | h | β− | 0.073 | 100.0 |
| 29 | Cu-60 | 23.7 | m | β+ | 0.872 | 49.0 |
| | | | | β+ | 1.325 | 15.0 |
| | | | | β+ | 0.840 | 11.6 |
| | | | | γ | 1.333 | 88.0 |
| | | | | γ | 1.792 | 45.4 |
| | | | | γ | 0.826 | 21.7 |
| 29 | Cu-61 | 3.3 | h | β+ | 0.524 | 51.0 |
| | | | | EC | 2.238 | 16.0 |
| | | | | EC | 1.582 | 11.1 |
| | | | | γ | 0.283 | 12.2 |
| | | | | γ | 0.656 | 10.8 |
| 29 | Cu-64 | 12.7 | h | β− | 0.191 | 38.5 |
| | | | | β+ | 0.278 | 17.6 |
| | | | | EC | 1.673 | 43.2 |
| 29 | Cu-67 | 61.8 | h | β− | 0.121 | 57.0 |
| | | | | β− | 0.154 | 22.0 |
| | | | | β− | 0.189 | 20.0 |
| | | | | γ | 0.185 | 48.7 |
| | | | | γ | 0.093 | 16.1 |
| 30 | Zn-62 | 9.2 | h | EC | 1.620 | 32.0 |
| | | | | EC | 1.071 | 31.0 |
| | | | | EC | 0.982 | 28.6 |
| | | | | γ | 0.597 | 26.0 |
| | | | | γ | 0.041 | 25.5 |
| | | | | γ | 0.548 | 15.3 |
| | | | | γ | 0.508 | 14.8 |
| 30 | Zn-63 | 38.5 | m | β+ | 1.042 | 80.3 |
| 30 | Zn-69 | 56.4 | m | β− | 0.322 | 100.0 |
| 30 | Zn-69m1 | 13.8 | h | γ | 0.439 | 94.9 |
| 30 | Zn-71m1 | 4.0 | h | β− | 0.569 | 87.8 |
| | | | | γ | 0.386 | 91.4 |
| | | | | γ | 0.487 | 61.2 |
| | | | | γ | 0.620 | 55.8 |
| | | | | γ | 0.512 | 28.7 |
| | | | | γ | 0.596 | 27.5 |
| 30 | Zn-72 | 46.5 | h | β− | 0.083 | 85.1 |
| | | | | β− | 0.067 | 14.7 |
| | | | | γ | 0.145 | 82.8 |
| 31 | Ga-65 | 15.2 | m | β+ | 0.938 | 58.0 |
| | | | | β+ | 0.895 | 10.2 |
| | | | | β+ | 0.966 | 10.0 |
| | | | | γ | 0.115 | 54.0 |
| | | | | γ | 0.061 | 11.4 |
| 31 | Ga-66 | 9.5 | h | β+ | 1.904 | 51.0 |
| | | | | EC | 1.420 | 26.0 |
| | | | | γ | 1.039 | 37.0 |
| | | | | γ | 2.752 | 22.7 |
| 31 | Ga-67 | 3.3 | d | EC | 0.908 | 52.5 |
| | | | | EC | 0.607 | 23.6 |
| | | | | EC | 0.816 | 22.7 |
| | | | | γ | 0.093 | 38.8 |
| | | | | γ | 0.185 | 21.4 |
| | | | | γ | 0.300 | 16.6 |
| 31 | Ga-68 | 67.7 | m | β+ | 0.836 | 87.7 |
| 31 | Ga-70 | 21.1 | m | β− | 0.648 | 98.9 |
| 31 | Ga-72 | 14.1 | h | β− | 0.344 | 28.9 |
| | | | | β− | 0.226 | 22.4 |
| | | | | β− | 0.219 | 15.7 |
| | | | | γ | 0.834 | 95.5 |
| | | | | γ | 2.202 | 26.9 |
| | | | | γ | 0.630 | 26.1 |
| | | | | γ | 2.508 | 13.3 |
| | | | | γ | 0.894 | 10.1 |
| 31 | Ga-73 | 4.9 | h | β− | 0.461 | 78.6 |
| | | | | γ | 0.297 | 79.8 |
| | | | | γ | 0.326 | 11.2 |
| | | | | γ | 0.053 | 10.5 |
| 32 | Ge-66 | 2.3 | h | β+ | 0.299 | 12.0 |
| | | | | EC | 1.718 | 36.3 |
| | | | | EC | 1.563 | 19.5 |
| | | | | γ | 0.044 | 29.1 |
| | | | | γ | 0.382 | 28.3 |
| | | | | γ | 0.109 | 10.6 |
| | | | | γ | 0.273 | 10.6 |
| 32 | Ge-67 | 18.9 | m | β+ | 1.368 | 74.0 |
| | | | | γ | 0.167 | 84.0 |
| 32 | Ge-69 | 39.1 | h | β+ | 0.522 | 21.0 |
| | | | | EC | 1.120 | 37.0 |
| | | | | EC | 1.355 | 11.5 |
| | | | | EC | 1.653 | 10.0 |
| | | | | EC | 2.227 | 10.0 |
| | | | | γ | 1.107 | 36.0 |
| | | | | γ | 0.574 | 13.3 |
| | | | | γ | 0.872 | 11.9 |
| 32 | Ge-71 | 11.4 | d | EC | 0.233 | 100.0 |
| 32 | Ge-75 | 82.8 | m | β− | 0.436 | 87.1 |
| | | | | β− | 0.323 | 11.5 |
| | | | | γ | 0.265 | 11.4 |
| 32 | Ge-77 | 11.2 | h | β− | 0.839 | 21.4 |
| | | | | β− | 0.584 | 19.2 |
| | | | | β− | 0.912 | 16.0 |
| | | | | γ | 0.264 | 53.3 |
| | | | | γ | 0.211 | 30.0 |
| | | | | γ | 0.216 | 27.9 |
| | | | | γ | 0.416 | 22.7 |
| | | | | γ | 0.558 | 16.8 |
| | | | | γ | 0.367 | 14.5 |
| 32 | Ge-78 | 88.0 | m | β− | 0.227 | 96.0 |
| | | | | γ | 0.277 | 96.0 |
| 33 | As-69 | 15.2 | m | β+ | 1.349 | 73.3 |
| | | | | β+ | 1.238 | 14.0 |
| | | | | γ | 0.233 | 10.9 |
| 33 | As-70 | 52.6 | m | β+ | 0.949 | 35.6 |
| | | | | β+ | 0.954 | 31.9 |
| | | | | γ | 1.039 | 82.0 |
| | | | | γ | 0.668 | 22.1 |
| | | | | γ | 0.744 | 22.1 |
| | | | | γ | 1.114 | 20.6 |
| | | | | γ | 0.595 | 18.8 |
| | | | | γ | 1.708 | 17.5 |
| | | | | γ | 2.019 | 16.6 |
| | | | | γ | 0.906 | 11.2 |
| 33 | As-71 | 65.3 | h | β+ | 0.352 | 27.9 |
| | | | | EC | 1.834 | 56.0 |
| | | | | γ | 0.175 | 82.4 |
| 33 | As-72 | 26.0 | h | β+ | 1.117 | 64.2 |
| | | | | β+ | 1.529 | 16.3 |
| | | | | γ | 0.834 | 81.0 |
| 33 | As-74 | 17.8 | d | β− | 0.531 | 19.0 |
| | | | | β− | 0.243 | 15.4 |
| | | | | β+ | 0.408 | 26.1 |
| | | | | EC | 1.963 | 33.1 |
| | | | | γ | 0.596 | 59.0 |
| | | | | γ | 0.635 | 15.4 |
| 33 | As-76 | 26.2 | h | β− | 1.264 | 51.0 |
| | | | | β− | 0.993 | 35.2 |
| | | | | γ | 0.559 | 45.0 |
| 33 | As-77 | 38.8 | h | β− | 0.229 | 97.0 |
| 33 | As-78 | 90.7 | m | β− | 1.856 | 32.0 |
| | | | | β− | 1.560 | 18.5 |
| | | | | β− | 0.607 | 15.9 |
| | | | | β− | 1.228 | 14.6 |
| | | | | γ | 0.614 | 54.0 |
| | | | | γ | 0.695 | 16.7 |
| | | | | γ | 1.309 | 13.0 |
| 34 | Se-70 | 41.1 | m | β+ | 0.569 | 27.2 |
| | | | | β+ | 0.402 | 18.2 |
| | | | | EC | 1.952 | 26.7 |
| | | | | EC | 2.328 | 13.2 |
| | | | | γ | 0.050 | 35.8 |
| | | | | γ | 0.426 | 29.1 |
| 34 | Se-72 | 8.4 | d | EC | 0.315 | 100.0 |
| | | | | γ | 0.046 | 57.2 |
| 34 | Se-73 | 7.2 | h | β+ | 0.555 | 63.9 |
| | | | | EC | 2.297 | 33.4 |
| | | | | γ | 0.361 | 97.0 |
| | | | | γ | 0.067 | 70.0 |
| 34 | Se-73m1 | 39.8 | m | β+ | 0.760 | 12.8 |
| 34 | Se-81 | 18.5 | m | β− | 0.614 | 98.7 |
| 34 | Se-81m1 | 57.3 | m | γ | 0.103 | 12.8 |
| 34 | Se-83 | 22.3 | m | β− | 0.369 | 30.7 |
| | | | | β− | 0.349 | 26.3 |
| | | | | β− | 0.331 | 16.4 |
| | | | | β− | 0.245 | 10.3 |
| | | | | γ | 0.357 | 69.4 |
| | | | | γ | 0.510 | 42.8 |
| | | | | γ | 0.225 | 29.5 |
| | | | | γ | 0.718 | 14.7 |
| | | | | γ | 0.799 | 14.6 |
| | | | | γ | 0.837 | 11.5 |
| 35 | Br-74 | 25.4 | m | β+ | 1.182 | 17.6 |
| | | | | β+ | 0.931 | 10.2 |
| | | | | γ | 0.635 | 64.1 |
| | | | | γ | 0.219 | 18.1 |
| | | | | γ | 0.634 | 14.1 |
| 35 | Br-74m1 | 46.0 | m | β+ | 2.445 | 11.0 |
| | | | | γ | 0.635 | 91.2 |
| | | | | γ | 0.728 | 35.6 |
| | | | | γ | 0.634 | 16.4 |
| 35 | Br-75 | 96.7 | m | β+ | 0.773 | 53.0 |
| | | | | EC | 2.775 | 11.0 |
| | | | | γ | 0.287 | 88.0 |
| 35 | Br-76 | 16.2 | h | β+ | 1.532 | 25.8 |
| | | | | EC | 1.893 | 13.5 |
| | | | | γ | 0.559 | 74.0 |
| | | | | γ | 0.657 | 15.9 |
| | | | | γ | 1.854 | 14.7 |
| 35 | Br-77 | 57.0 | h | EC | 1.365 | 43.8 |
| | | | | EC | 0.844 | 18.6 |
| | | | | EC | 1.126 | 15.3 |
| | | | | EC | 0.547 | 10.0 |
| | | | | γ | 0.239 | 23.1 |
| | | | | γ | 0.521 | 22.4 |
| 35 | Br-80 | 17.7 | m | β− | 0.804 | 85.0 |
| 35 | Br-80m1 | 4.4 | h | γ | 0.037 | 39.1 |
| 35 | Br-82 | 35.3 | h | β− | 0.138 | 99.1 |
| | | | | γ | 0.777 | 83.6 |
| | | | | γ | 0.554 | 71.7 |
| | | | | γ | 0.619 | 43.7 |
| | | | | γ | 0.698 | 28.4 |
| | | | | γ | 1.044 | 27.6 |
| | | | | γ | 1.317 | 26.9 |
| | | | | γ | 0.828 | 24.2 |
| | | | | γ | 1.475 | 16.4 |
| 35 | Br-83 | 2.4 | h | β− | 0.330 | 98.6 |
| 35 | Br-84 | 31.8 | m | β− | 2.051 | 33.0 |
| | | | | β− | 1.629 | 13.5 |
| | | | | β− | 1.145 | 11.7 |
| | | | | β− | 0.235 | 11.5 |
| | | | | γ | 0.882 | 42.0 |
| | | | | γ | 1.898 | 14.6 |
| 36 | Kr-74 | 11.5 | m | β+ | 0.725 | 32.0 |
| | | | | β+ | 0.768 | 25.0 |
| | | | | β+ | 0.824 | 10.0 |
| | | | | γ | 0.090 | 31.0 |
| | | | | γ | 0.203 | 18.0 |
| 36 | Kr-76 | 14.8 | h | EC | 0.995 | 55.0 |
| | | | | EC | 0.859 | 26.2 |
| | | | | EC | 0.956 | 10.4 |
| | | | | γ | 0.316 | 39.0 |
| | | | | γ | 0.270 | 21.0 |
| | | | | γ | 0.046 | 19.5 |
| | | | | γ | 0.407 | 12.1 |
| 36 | Kr-77 | 74.4 | m | β+ | 0.847 | 41.8 |
| | | | | β+ | 0.780 | 33.6 |
| | | | | γ | 0.130 | 81.0 |
| | | | | γ | 0.147 | 37.3 |
| 36 | Kr-79 | 35.0 | h | EC | 1.626 | 55.9 |
| | | | | EC | 1.020 | 12.1 |
| | | | | EC | 1.365 | 11.6 |
| | | | | γ | 0.261 | 12.7 |
| 36 | Kr-85 | 4.5 | h | β− | 0.290 | 78.5 |
| | | | | γ | 0.151 | 75.2 |
| | | | | γ | 0.305 | 14.0 |
| 36 | Kr-87 | 76.3 | m | β− | 1.502 | 41.0 |
| | | | | β− | 1.694 | 30.5 |
| | | | | γ | 0.403 | 50.0 |
| 36 | Kr-88 | 2.8 | h | β− | 0.167 | 67.0 |
| | | | | β− | 1.235 | 14.0 |
| | | | | γ | 2.392 | 34.6 |
| | | | | γ | 0.196 | 26.0 |
| | | | | γ | 2.196 | 13.2 |
| | | | | γ | 0.835 | 13.0 |
| | | | | γ | 1.530 | 10.9 |
| 37 | Rb-78 | 17.7 | m | β+ | 1.039 | 15.9 |
| | | | | β+ | 1.253 | 14.9 |
| | | | | γ | 0.455 | 62.7 |
| | | | | γ | 0.693 | 12.6 |
| | | | | γ | 0.562 | 11.4 |
| | | | | γ | 3.438 | 10.8 |
| 37 | Rb-79 | 22.9 | m | β+ | 0.855 | 42.0 |
| | | | | β+ | 1.039 | 14.2 |
| | | | | β+ | 1.114 | 10.9 |
| | | | | β+ | 0.825 | 10.4 |
| | | | | γ | 0.688 | 23.4 |
| | | | | γ | 0.183 | 19.4 |
| | | | | γ | 0.143 | 14.1 |
| | | | | γ | 0.130 | 10.9 |
| | | | | γ | 0.505 | 10.8 |
| | | | | γ | 0.147 | 10.6 |
| 37 | Rb-81 | 4.6 | h | β+ | 0.448 | 25.0 |
| | | | | EC | 2.072 | 39.0 |
| | | | | EC | 1.597 | 19.9 |
| | | | | γ | 0.190 | 64.9 |
| | | | | γ | 0.446 | 23.5 |
| 37 | Rb-82 | 6.5 | h | β+ | 0.353 | 19.7 |
| | | | | EC | 1.825 | 66.7 |
| | | | | γ | 0.777 | 84.4 |
| | | | | γ | 0.554 | 62.4 |
| | | | | γ | 0.619 | 38.0 |
| | | | | γ | 1.044 | 32.1 |
| | | | | γ | 0.698 | 26.3 |
| | | | | γ | 1.317 | 23.7 |
| | | | | γ | 0.828 | 21.0 |
| | | | | γ | 1.475 | 15.5 |
| 37 | Rb-84 | 32.8 | d | β+ | 0.759 | 13.1 |
| | | | | β+ | 0.341 | 12.6 |
| | | | | EC | 0.782 | 56.0 |
| | | | | EC | 1.658 | 13.4 |
| | | | | γ | 0.882 | 68.9 |
| 37 | Rb-84m1 | 20.3 | m | γ | 0.248 | 63.0 |
| | | | | γ | 0.464 | 33.1 |
| | | | | γ | 0.216 | 31.3 |
| 37 | Rb-86 | 18.6 | d | β− | 0.710 | 91.4 |
| 37 | Rb-88 | 17.8 | m | β− | 2.371 | 76.5 |
| | | | | β− | 1.069 | 13.6 |
| | | | | γ | 1.836 | 22.8 |
| | | | | γ | 0.898 | 14.4 |
| 37 | Rb-89 | 15.3 | m | β− | 0.900 | 37.0 |
| | | | | β− | 0.471 | 35.7 |
| | | | | β− | 1.985 | 18.8 |
| | | | | γ | 1.032 | 63.0 |
| | | | | γ | 1.248 | 46.0 |
| | | | | γ | 2.196 | 14.5 |
| | | | | γ | 0.658 | 10.8 |
| | | | | γ | 2.570 | 10.7 |
| | | | | γ | 0.948 | 10.0 |
| 38 | Sr-80 | 106.3 | m | EC | 1.276 | 42.0 |
| | | | | EC | 1.865 | 31.0 |
| | | | | EC | 1.312 | 12.0 |
| | | | | γ | 0.589 | 39.0 |
| | | | | γ | 0.175 | 10.1 |
| 38 | Sr-81 | 22.3 | m | β+ | 1.169 | 28.0 |
| | | | | β+ | 1.102 | 16.0 |
| | | | | β+ | 1.310 | 13.0 |
| | | | | γ | 0.154 | 34.0 |
| | | | | γ | 0.148 | 30.0 |
| | | | | γ | 0.443 | 17.5 |
| | | | | γ | 0.188 | 15.4 |
| 38 | Sr-82 | 25.4 | d | EC | 0.178 | 100.0 |
| 38 | Sr-83 | 32.4 | h | β+ | 0.548 | 12.4 |
| | | | | β+ | 0.529 | 11.0 |
| | | | | EC | 1.468 | 25.1 |
| | | | | EC | 2.231 | 12.0 |
| | | | | EC | 1.849 | 11.5 |
| | | | | EC | 2.273 | 11.1 |
| | | | | γ | 0.763 | 26.7 |
| | | | | γ | 0.382 | 14.0 |
| 38 | Sr-85m1 | 67.6 | m | EC | 1.152 | 13.3 |
| | | | | γ | 0.232 | 83.9 |
| | | | | γ | 0.151 | 12.8 |
| 38 | Sr-87 | 2.8 | h | γ | 0.389 | 82.2 |
| 38 | Sr-89 | 50.6 | d | β− | 0.587 | 100 |
| 38 | Sr-91 | 9.7 | h | β− | 0.405 | 34.8 |
| | | | | β− | 1.128 | 28.6 |
| | | | | β− | 0.525 | 25.1 |
| | | | | γ | 1.024 | 33.5 |
| | | | | γ | 0.750 | 23.7 |
| 38 | Sr-92 | 2.6 | h | β− | 0.182 | 97.0 |
| | | | | γ | 1.384 | 90.0 |
| 39 | Y-84 | 39.5 | m | β+ | 1.321 | 28.0 |
| | | | | β+ | 1.104 | 14.5 |
| | | | | γ | 0.793 | 98.3 |
| | | | | γ | 0.974 | 78.0 |
| | | | | γ | 1.040 | 56.0 |
| | | | | γ | 0.661 | 11.3 |
| | | | | γ | 0.463 | 10.1 |
| 39 | Y-85 | 2.7 | h | β+ | 0.659 | 38.0 |
| | | | | β+ | 0.889 | 24.0 |
| | | | | EC | 2.518 | 21.0 |
| | | | | γ | 0.232 | 84.0 |
| | | | | γ | 0.504 | 60.0 |
| 39 | Y-85m1 | 4.9 | h | β+ | 1.008 | 52.0 |
| | | | | EC | 1.157 | 11.7 |
| | | | | γ | 0.232 | 22.8 |
| 39 | Y-86 | 14.7 | h | β+ | 0.535 | 11.9 |
| | | | | EC | 1.409 | 14.2 |
| | | | | EC | 2.243 | 12.9 |
| | | | | γ | 1.077 | 82.5 |
| | | | | γ | 0.628 | 32.6 |
| | | | | γ | 1.153 | 30.5 |
| | | | | γ | 0.777 | 22.4 |
| | | | | γ | 1.921 | 20.8 |
| | | | | γ | 1.854 | 17.2 |
| | | | | γ | 0.443 | 16.9 |
| | | | | γ | 0.703 | 15.4 |
| 39 | Y-86m1 | 47.4 | m | γ | 0.208 | 93.8 |
| 39 | Y-87 | 79.8 | h | EC | 0.988 | 93.4 |
| | | | | γ | 0.485 | 89.8 |
| | | | | γ | 0.389 | 82.2 |
| 39 | Y-87m1 | 13.4 | h | γ | 0.381 | 78.1 |
| 39 | Y-90 | 64.0 | h | β− | 0.934 | 100.0 |
| 39 | Y-90m1 | 3.2 | h | γ | 0.203 | 97.3 |
| | | | | γ | 0.480 | 90.7 |
| 39 | Y-91m1 | 49.7 | m | γ | 0.556 | 95.0 |
| 39 | Y-92 | 3.5 | h | β− | 1.567 | 85.7 |
| | | | | γ | 0.934 | 13.9 |
| 39 | Y-93 | 10.2 | h | β− | 1.217 | 89.5 |
| 39 | Y-94 | 18.7 | m | β− | 2.174 | 41.0 |
| | | | | β− | 1.741 | 39.6 |
| | | | | γ | 0.919 | 56.0 |
| 39 | Y-95 | 10.3 | m | β− | 1.936 | 64.0 |
| | | | | γ | 0.954 | 15.8 |
| 40 | Zr-86 | 16.5 | h | EC | 1.043 | 95.0 |
| | | | | γ | 0.243 | 95.8 |
| | | | | γ | 0.029 | 21.6 |
| 40 | Zr-87 | 1.7 | h | β+ | 1.014 | 80.5 |
| | | | | EC | 3.291 | 13.9 |
| 40 | Zr-89 | 78.4 | h | β+ | 0.396 | 22.7 |
| | | | | EC | 1.924 | 76.2 |
| | | | | γ | 0.909 | 99.0 |
| 40 | Zr-97 | 16.7 | h | β− | 0.757 | 87.8 |
| | | | | γ | 0.743 | 93.1 |
| 41 | Nb-88 | 14.5 | m | β+ | 1.374 | 54.0 |
| | | | | β+ | 1.612 | 25.0 |
| | | | | γ | 1.083 | 103.0 |
| | | | | γ | 1.057 | 100.0 |
| | | | | γ | 0.671 | 64.0 |
| | | | | γ | 0.503 | 60.0 |
| | | | | γ | 0.399 | 31.8 |
| | | | | γ | 0.272 | 30.1 |
| | | | | γ | 0.077 | 22.4 |
| 41 | Nb-89 | 2.0 | h | β+ | 1.453 | 74.0 |
| 41 | Nb-89m1 | 66.0 | m | β+ | 0.978 | 62.0 |
| | | | | β+ | 1.215 | 15.0 |
| | | | | EC | 3.212 | 13.0 |
| | | | | γ | 0.588 | 95.6 |
| | | | | γ | 0.507 | 81.0 |
| 41 | Nb-90 | 14.6 | h | β+ | 0.662 | 51.1 |
| | | | | EC | 2.522 | 35.9 |
| | | | | γ | 1.129 | 92.7 |
| | | | | γ | 2.319 | 82.0 |
| | | | | γ | 0.141 | 66.8 |
| | | | | γ | 2.186 | 18.0 |
| 41 | Nb-92 | 10.2 | d | EC | 1.207 | 97.3 |
| | | | | γ | 0.934 | 99.2 |
| 41 | Nb-95 | 35.0 | d | β− | 0.043 | 100.0 |
| | | | | γ | 0.766 | 99.8 |
| 41 | Nb-95m1 | 3.6 | d | γ | 0.236 | 24.8 |
| 41 | Nb-96 | 23.4 | h | β− | 0.251 | 96.7 |
| | | | | γ | 0.778 | 96.5 |
| | | | | γ | 0.569 | 58.0 |
| | | | | γ | 1.091 | 48.5 |
| | | | | γ | 0.460 | 26.6 |
| | | | | γ | 0.850 | 20.5 |
| | | | | γ | 1.200 | 20.0 |
| | | | | γ | 0.810 | 11.1 |
| 41 | Nb-97 | 72.1 | m | β− | 0.471 | 98.4 |
| | | | | γ | 0.658 | 98.2 |
| 41 | Nb-98m1 | 51.3 | m | β− | 0.791 | 27.9 |
| | | | | β− | 1.001 | 15.9 |
| | | | | β− | 0.550 | 10.4 |
| | | | | γ | 0.787 | 93.4 |
| | | | | γ | 0.723 | 73.8 |
| | | | | γ | 1.169 | 17.8 |
| | | | | γ | 0.834 | 10.8 |
| 42 | Mo-90 | 5.6 | h | β+ | 0.477 | 24.9 |
| | | | | EC | 2.107 | 56.0 |
| | | | | γ | 0.257 | 78.0 |
| | | | | γ | 0.122 | 64.0 |
| 42 | Mo-91 | 15.5 | m | β+ | 1.550 | 93.3 |
| 42 | Mo-93m1 | 6.9 | h | γ | 0.685 | 99.9 |
| | | | | γ | 1.477 | 99.1 |
| | | | | γ | 0.263 | 57.4 |
| 42 | Mo-99 | 65.9 | h | β− | 0.443 | 82.2 |
| | | | | β− | 0.133 | 16.4 |
| | | | | γ | 0.740 | 12.2 |
| 42 | Mo-101 | 14.6 | m | β− | 0.261 | 20.6 |
| | | | | β− | 0.295 | 15.0 |
| | | | | β− | 1.079 | 12.8 |
| | | | | γ | 0.590 | 19.2 |
| | | | | γ | 0.192 | 18.2 |
| | | | | γ | 1.012 | 13.0 |
| | | | | γ | 0.506 | 11.6 |
| 42 | Mo-102 | 11.3 | m | β− | 0.358 | 94.1 |
| 43 | Tc-93 | 2.8 | h | EC | 1.838 | 56.7 |
| | | | | EC | 1.681 | 22.2 |
| | | | | γ | 1.363 | 66.2 |
| | | | | γ | 1.520 | 24.4 |
| 43 | Tc-93m1 | 43.5 | m | EC | 0.948 | 14.3 |
| | | | | γ | 0.392 | 58.3 |
| | | | | γ | 2.645 | 14.3 |
| 43 | Tc-94 | 293.0 | m | β+ | 0.358 | 10.5 |
| | | | | EC | 1.833 | 70.8 |
| | | | | γ | 0.871 | 99.9 |
| | | | | γ | 0.703 | 99.6 |
| | | | | γ | 0.850 | 95.7 |
| 43 | Tc-94m1 | 52.0 | m | β+ | 1.094 | 67.6 |
| | | | | EC | 3.461 | 12.8 |
| | | | | γ | 0.871 | 94.2 |
| 43 | Tc-95 | 20.0 | h | EC | 0.925 | 93.8 |
| | | | | γ | 0.766 | 93.8 |
| 43 | Tc-96 | 4.3 | d | EC | 0.532 | 79.0 |
| | | | | EC | 0.218 | 19.5 |
| | | | | γ | 0.778 | 99.8 |
| | | | | γ | 0.850 | 98.0 |
| | | | | γ | 0.813 | 82.0 |
| | | | | γ | 1.127 | 15.2 |
| 43 | Tc-99m1 | 6.0 | h | γ | 0.141 | 89.0 |
| 43 | Tc-101 | 14.2 | m | β− | 0.487 | 90.3 |
| | | | | γ | 0.307 | 89.0 |
| 43 | Tc-104 | 18.3 | m | β− | 2.326 | 16.0 |
| | | | | γ | 0.358 | 89.0 |
| | | | | γ | 0.531 | 15.6 |
| | | | | γ | 0.535 | 14.7 |
| | | | | γ | 0.884 | 10.9 |
| | | | | γ | 0.893 | 10.2 |
| 44 | Ru-94 | 51.8 | m | EC | 1.144 | 73.0 |
| | | | | EC | 0.619 | 27.0 |
| | | | | γ | 0.367 | 75.0 |
| | | | | γ | 0.891 | 25.0 |
| 44 | Ru-95 | 1.6 | h | β+ | 0.533 | 12.6 |
| | | | | EC | 2.222 | 24.4 |
| | | | | EC | 1.132 | 24.0 |
| | | | | γ | 0.336 | 69.9 |
| | | | | γ | 1.097 | 20.9 |
| | | | | γ | 0.627 | 17.8 |
| 44 | Ru-97 | 2.8 | d | EC | 0.892 | 87.7 |
| | | | | EC | 0.784 | 11.0 |
| | | | | γ | 0.216 | 85.6 |
| | | | | γ | 0.324 | 10.8 |
| 44 | Ru-103 | 39.2 | d | β− | 0.064 | 92.0 |
| | | | | γ | 0.497 | 91.0 |
| 44 | Ru-105 | 4.4 | h | β− | 0.432 | 47.8 |
| | | | | β− | 0.398 | 18.8 |
| | | | | β− | 0.406 | 16.9 |
| | | | | γ | 0.724 | 47.3 |
| | | | | γ | 0.469 | 17.5 |
| | | | | γ | 0.676 | 15.7 |
| | | | | γ | 0.316 | 11.1 |
| 45 | Rh-97 | 30.7 | m | β+ | 0.923 | 49.1 |
| | | | | EC | 3.098 | 19.1 |
| | | | | γ | 0.422 | 74.6 |
| | | | | γ | 0.840 | 12.0 |
| 45 | Rh-97m1 | 46.2 | m | β+ | 1.155 | 10.4 |
| | | | | EC | 1.581 | 20.4 |
| | | | | EC | 1.533 | 18.5 |
| | | | | γ | 0.189 | 48.5 |
| | | | | γ | 2.246 | 13.8 |
| | | | | γ | 0.422 | 12.7 |
| 45 | Rh-99 | 16.1 | d | EC | 1.485 | 45.0 |
| | | | | EC | 1.660 | 31.3 |
| | | | | γ | 0.528 | 37.9 |
| | | | | γ | 0.353 | 34.5 |
| | | | | γ | 0.090 | 33.4 |
| 45 | Rh-99m1 | 4.7 | h | EC | 1.768 | 60.0 |
| | | | | EC | 1.491 | 13.9 |
| | | | | EC | 0.847 | 12.7 |
| | | | | γ | 0.341 | 72.0 |
| | | | | γ | 0.618 | 12.3 |
| | | | | γ | 1.261 | 11.4 |
| 45 | Rh-100 | 20.8 | h | EC | 0.719 | 68.6 |
| | | | | EC | 1.166 | 19.9 |
| | | | | γ | 0.540 | 80.6 |
| | | | | γ | 2.376 | 32.6 |
| | | | | γ | 0.823 | 21.1 |
| | | | | γ | 1.553 | 20.7 |
| | | | | γ | 1.362 | 15.4 |
| | | | | γ | 1.107 | 13.6 |
| | | | | γ | 0.446 | 12.0 |
| | | | | γ | 1.930 | 11.6 |
| 45 | Rh-101m1 | 4.3 | d | EC | 0.393 | 83.0 |
| | | | | γ | 0.307 | 81.0 |
| 45 | Rh-105 | 35.4 | h | β− | 0.179 | 75.0 |
| | | | | β− | 0.070 | 19.7 |
| | | | | γ | 0.319 | 19.1 |
| 45 | Rh-106m1 | 131.0 | m | β− | 0.318 | 85.0 |
| | | | | β− | 0.240 | 14.5 |
| | | | | γ | 0.512 | 85.0 |
| | | | | γ | 1.047 | 30.4 |
| | | | | γ | 0.717 | 28.9 |
| | | | | γ | 0.451 | 24.2 |
| | | | | γ | 0.616 | 20.2 |
| | | | | γ | 0.749 | 19.3 |
| | | | | γ | 1.529 | 17.5 |
| | | | | γ | 1.128 | 13.7 |
| | | | | γ | 0.825 | 13.6 |
| | | | | γ | 0.429 | 13.3 |
| | | | | γ | 0.805 | 13.0 |
| | | | | γ | 0.406 | 11.6 |
| | | | | γ | 1.201 | 11.4 |
| 45 | Rh-107 | 21.7 | m | β− | 0.437 | 64.0 |
| | | | | β− | 0.569 | 13.0 |
| | | | | γ | 0.303 | 66.0 |
| 46 | Pd-98 | 17.7 | m | EC | 1.755 | 61.0 |
| | | | | EC | 1.029 | 24.0 |
| | | | | γ | 0.112 | 58.0 |
| | | | | γ | 0.662 | 19.7 |
| | | | | γ | 0.107 | 13.9 |
| 46 | Pd-99 | 21.4 | m | β+ | 0.972 | 36.0 |
| | | | | EC | 3.198 | 13.0 |
| | | | | γ | 0.136 | 73.0 |
| | | | | γ | 0.264 | 15.0 |
| 46 | Pd-100 | 3.6 | d | EC | 0.199 | 92.0 |
| | | | | γ | 0.084 | 52.0 |
| | | | | γ | 0.075 | 48.0 |
| 46 | Pd-101 | 8.5 | h | EC | 1.798 | 52.2 |
| | | | | EC | 1.232 | 21.6 |
| | | | | EC | 1.510 | 11.4 |
| | | | | γ | 0.296 | 19.2 |
| | | | | γ | 0.590 | 12.1 |
| 46 | Pd-103 | 17.0 | d | EC | 0.503 | 99.9 |
| 46 | Pd-109 | 13.6 | h | β− | 0.360 | 100.0 |
| 46 | Pd-111 | 23.4 | m | β− | 0.852 | 95.3 |
| 46 | Pd-111m1 | 5.5 | h | γ | 0.172 | 46.0 |
| 46 | Pd-112 | 21.0 | h | β− | 0.077 | 100.0 |
| | | | | γ | 0.019 | 27.0 |
| 47 | Ag-101 | 11.1 | m | β+ | 1.319 | 37.0 |
| | | | | β+ | 0.896 | 11.2 |
| | | | | γ | 0.261 | 52.6 |
| | | | | γ | 0.588 | 10.0 |
| 47 | Ag-103 | 65.7 | m | β+ | 0.614 | 14.8 |
| | | | | EC | 2.421 | 25.2 |
| | | | | EC | 1.414 | 20.5 |
| | | | | γ | 0.119 | 31.2 |
| | | | | γ | 0.148 | 28.3 |
| | | | | γ | 0.267 | 13.3 |
| 47 | Ag-104 | 69.2 | m | EC | 2.014 | 29.0 |
| | | | | EC | 2.197 | 12.7 |
| | | | | EC | 2.097 | 12.2 |
| | | | | γ | 0.556 | 92.6 |
| | | | | γ | 0.768 | 65.7 |
| | | | | γ | 0.942 | 25.0 |
| | | | | γ | 0.926 | 12.5 |
| | | | | γ | 0.858 | 10.4 |
| 47 | Ag-104m1 | 33.5 | m | β+ | 1.222 | 58.0 |
| | | | | EC | 3.730 | 12.0 |
| | | | | γ | 0.556 | 90.0 |
| 47 | Ag-105 | 41.3 | d | EC | 1.000 | 65.0 |
| | | | | EC | 0.257 | 17.2 |
| | | | | γ | 0.345 | 41.4 |
| | | | | γ | 0.280 | 30.2 |
| | | | | γ | 0.645 | 11.1 |
| | | | | γ | 0.064 | 10.5 |
| | | | | γ | 0.443 | 10.5 |
| 47 | Ag-106 | 24.0 | m | β+ | 0.868 | 52.6 |
| | | | | EC | 2.965 | 30.3 |
| | | | | γ | 0.512 | 17.0 |
| 47 | Ag-106m1 | 8.3 | d | EC | 0.298 | 92.0 |
| | | | | γ | 0.512 | 88.0 |
| | | | | γ | 1.046 | 29.6 |
| | | | | γ | 0.717 | 28.9 |
| | | | | γ | 0.451 | 28.2 |
| | | | | γ | 0.616 | 21.6 |
| | | | | γ | 0.748 | 20.6 |
| | | | | γ | 1.528 | 16.3 |
| | | | | γ | 0.825 | 15.3 |
| | | | | γ | 0.406 | 13.4 |
| | | | | γ | 0.430 | 13.2 |
| | | | | γ | 0.804 | 12.4 |
| | | | | γ | 1.128 | 11.8 |
| | | | | γ | 1.199 | 11.2 |
| 47 | Ag-111 | 7.5 | d | β− | 0.360 | 92.0 |
| 47 | Ag-112 | 3.1 | h | β− | 1.703 | 54.0 |
| | | | | β− | 1.426 | 20.0 |
| | | | | γ | 0.618 | 42.0 |
| 47 | Ag-113 | 5.4 | h | β− | 0.794 | 85.0 |
| | | | | γ | 0.299 | 10.0 |
| 47 | Ag-115 | 20.0 | m | β− | 1.296 | 60.0 |
| | | | | β− | 1.189 | 12.5 |
| | | | | γ | 0.229 | 18.0 |
| 48 | Cd-104 | 57.7 | m | EC | 1.046 | 71.8 |
| | | | | EC | 0.421 | 28.5 |
| | | | | γ | 0.084 | 47.0 |
| | | | | γ | 0.709 | 19.5 |
| 48 | Cd-105 | 55.5 | m | β+ | 0.754 | 27.3 |
| | | | | EC | 2.713 | 26.7 |
| 48 | Cd-107 | 6.5 | h | EC | 1.324 | 99.7 |
| 48 | Cd-111 | 48.5 | m | γ | 0.245 | 94.0 |
| | | | | γ | 0.151 | 29.1 |
| 48 | Cd-115 | 53.5 | h | β− | 0.394 | 62.6 |
| | | | | β− | 0.184 | 33.1 |
| | | | | γ | 0.528 | 27.4 |
| 48 | Cd-115m1 | 44.6 | d | β− | 0.618 | 97.0 |
| 48 | Cd-117 | 2.5 | h | β− | 0.202 | 32.0 |
| | | | | β− | 0.880 | 21.0 |
| | | | | β− | 0.683 | 13.2 |
| | | | | γ | 0.273 | 27.9 |
| | | | | γ | 1.303 | 18.4 |
| | | | | γ | 0.344 | 17.9 |
| | | | | γ | 1.577 | 11.2 |
| 48 | Cd-117m1 | 3.4 | h | β− | 0.216 | 48.0 |
| | | | | β− | 0.179 | 20.5 |
| | | | | γ | 1.997 | 26.2 |
| | | | | γ | 1.066 | 23.1 |
| | | | | γ | 0.564 | 14.7 |
| | | | | γ | 1.433 | 13.4 |
| | | | | γ | 1.029 | 11.7 |
| | | | | γ | 1.235 | 11.0 |
| 48 | Cd-118 | 50.3 | m | β− | 0.161 | 100.0 |
| 49 | In-107 | 32.4 | m | β+ | 1.020 | 27.0 |
| | | | | EC | 3.220 | 11.5 |
| | | | | γ | 0.205 | 47.2 |
| | | | | γ | 0.506 | 11.9 |
| | | | | γ | 0.321 | 10.2 |
| 49 | In-108m1 | 39.6 | m | β+ | 1.599 | 30.4 |
| | | | | γ | 0.633 | 76.4 |
| | | | | γ | 1.986 | 12.4 |
| 49 | In-109 | 4.2 | h | EC | 1.813 | 63.4 |
| | | | | γ | 0.203 | 74.2 |
| 49 | In-110 | 4.9 | h | EC | 0.756 | 43.8 |
| | | | | EC | 0.691 | 29.6 |
| | | | | γ | 0.658 | 98.0 |
| | | | | γ | 0.885 | 93.0 |
| | | | | γ | 0.937 | 68.4 |
| | | | | γ | 0.707 | 29.5 |
| | | | | γ | 0.642 | 26.0 |
| | | | | γ | 0.997 | 10.5 |
| 49 | In-110m1 | 69.1 | m | β+ | 1.015 | 60.7 |
| | | | | EC | 3.282 | 26.5 |
| | | | | γ | 0.658 | 97.7 |
| 49 | In-111 | 2.8 | d | EC | 0.445 | 100.0 |
| | | | | γ | 0.245 | 94.1 |
| | | | | γ | 0.171 | 90.6 |
| 49 | In-112 | 14.9 | m | β− | 0.215 | 38.0 |
| | | | | β+ | 0.697 | 23.3 |
| | | | | EC | 2.582 | 32.0 |
| | | | | γ | 0.157 | 13.3 |
| 49 | In-113 | 99.5 | m | γ | 0.392 | 64.9 |
| 49 | In-114m1 | 49.5 | d | γ | 0.190 | 15.6 |
| 49 | In-115m1 | 4.5 | h | β− | 0.279 | 100.0 |
| | | | | γ | 0.336 | 45.9 |
| 49 | In-116m1 | 54.3 | m | β− | 0.352 | 54.2 |
| | | | | β− | 0.296 | 32.5 |
| | | | | β− | 0.190 | 10.3 |
| | | | | γ | 1.294 | 84.8 |
| | | | | γ | 1.097 | 58.5 |
| | | | | γ | 0.417 | 27.2 |
| | | | | γ | 2.112 | 15.1 |
| | | | | γ | 0.819 | 12.1 |
| 49 | In-117 | 43.2 | m | β− | 0.245 | 99.8 |
| | | | | γ | 0.553 | 100.0 |
| | | | | γ | 0.159 | 87.0 |
| 49 | In-117m1 | 116.2 | m | β− | 0.680 | 18.3 |
| | | | | γ | 0.315 | 19.1 |
| | | | | γ | 0.159 | 15.9 |
| 49 | In-119m1 | 18.0 | m | β− | 1.094 | 61.2 |
| | | | | β− | 1.083 | 33.5 |
| 50 | Sn-108 | 10.3 | m | EC | 1.376 | 87.5 |
| | | | | γ | 0.396 | 64.3 |
| | | | | γ | 0.273 | 45.5 |
| | | | | γ | 0.669 | 22.6 |
| | | | | γ | 0.168 | 19.9 |
| | | | | γ | 0.104 | 13.8 |
| 50 | Sn-109 | 18.1 | m | γ | 0.650 | 29.6 |
| | | | | γ | 1.099 | 29.3 |
| | | | | γ | 1.321 | 11.5 |
| 50 | Sn-110 | 4.2 | h | EC | 0.288 | 100.0 |
| | | | | γ | 0.280 | 97.1 |
| 50 | Sn-111 | 35.3 | m | β+ | 0.635 | 30.2 |
| | | | | EC | 2.451 | 62.7 |
| 50 | Sn-117 | 14.0 | d | γ | 0.159 | 86.4 |
| 50 | Sn-121 | 27.0 | h | β− | 0.116 | 100.0 |
| 50 | Sn-123m1 | 40.1 | m | β− | 0.459 | 99.9 |
| | | | | γ | 0.160 | 85.7 |
| 50 | Sn-125 | 9.6 | d | β− | 0.941 | 81.4 |
| | | | | γ | 1.067 | 10.0 |
| 50 | Sn-127 | 2.1 | h | β− | 1.325 | 22.0 |
| | | | | β− | 0.195 | 11.2 |
| | | | | γ | 1.114 | 38.0 |
| | | | | γ | 1.096 | 19.0 |
| | | | | γ | 0.823 | 11.0 |
| 50 | Sn-128 | 59.1 | m | β− | 0.211 | 84.0 |
| | | | | β− | 0.139 | 16.1 |
| | | | | γ | 0.482 | 59.0 |
| | | | | γ | 0.075 | 28.0 |
| | | | | γ | 0.557 | 16.5 |
| | | | | γ | 0.681 | 15.9 |
| | | | | γ | 0.046 | 13.0 |
| 51 | Sb-115 | 32.1 | m | β+ | 0.675 | 33.3 |
| | | | | EC | 2.530 | 63.0 |
| | | | | γ | 0.497 | 97.9 |
| 51 | Sb-116 | 15.8 | m | β+ | 1.076 | 40.0 |
| | | | | β+ | 0.652 | 13.0 |
| | | | | EC | 2.482 | 27.0 |
| | | | | EC | 3.413 | 18.0 |
| | | | | γ | 1.294 | 85.0 |
| | | | | γ | 0.932 | 24.8 |
| | | | | γ | 2.225 | 14.6 |
| 51 | Sb-116m1 | 60.3 | m | β+ | 0.619 | 24.0 |
| | | | | EC | 2.181 | 58.0 |
| | | | | EC | 1.880 | 12.6 |
| | | | | γ | 0.100 | 100.0 |
| | | | | γ | 0.407 | 100.0 |
| | | | | γ | 1.294 | 100.0 |
| | | | | γ | 0.973 | 74.0 |
| | | | | γ | 0.543 | 48.1 |
| | | | | γ | 0.136 | 28.5 |
| | | | | γ | 1.072 | 25.5 |
| | | | | γ | 0.844 | 11.2 |
| 51 | Sb-117 | 2.8 | h | EC | 1.586 | 97.2 |
| | | | | γ | 0.159 | 85.9 |
| 51 | Sb-118m1 | 5.0 | h | EC | 1.332 | 98.3 |
| | | | | γ | 1.230 | 100.0 |
| | | | | γ | 0.254 | 99.0 |
| | | | | γ | 1.051 | 97.0 |
| | | | | γ | 0.041 | 30.0 |
| 51 | Sb-119 | 38.2 | h | EC | 0.567 | 100.0 |
| | | | | γ | 0.024 | 16.5 |
| 51 | Sb-120 | 15.9 | m | β+ | 0.742 | 41.0 |
| | | | | EC | 2.681 | 57.3 |
| 51 | Sb-120m1 | 5.8 | d | EC | 0.199 | 100.0 |
| | | | | γ | 1.172 | 100.0 |
| | | | | γ | 1.023 | 99.4 |
| | | | | γ | 0.197 | 87.0 |
| | | | | γ | 0.090 | 79.5 |
| 51 | Sb-122 | 2.7 | d | β− | 0.524 | 66.7 |
| | | | | β− | 0.773 | 26.1 |
| | | | | γ | 0.564 | 70.7 |
| 51 | Sb-126 | 12.4 | d | β− | 0.146 | 29.0 |
| | | | | β− | 0.735 | 20.0 |
| | | | | β− | 0.419 | 16.0 |
| | | | | γ | 0.667 | 99.6 |
| | | | | γ | 0.695 | 99.6 |
| | | | | γ | 0.415 | 83.3 |
| | | | | γ | 0.721 | 53.8 |
| | | | | γ | 0.697 | 29.0 |
| | | | | γ | 0.857 | 17.6 |
| 51 | Sb-126m1 | 19.2 | m | β− | 0.743 | 83.0 |
| | | | | γ | 0.415 | 86.0 |
| | | | | γ | 0.666 | 86.0 |
| | | | | γ | 0.695 | 82.0 |
| 51 | Sb-127 | 3.9 | d | β− | 0.304 | 35.8 |
| | | | | β− | 0.391 | 23.4 |
| | | | | β− | 0.265 | 18.0 |
| | | | | γ | 0.686 | 36.8 |
| | | | | γ | 0.473 | 25.8 |
| | | | | γ | 0.784 | 15.1 |
| 51 | Sb-128 | 9.1 | h | β− | 0.792 | 19.0 |
| | | | | β− | 0.325 | 15.3 |
| | | | | β− | 0.434 | 13.0 |
| | | | | γ | 0.743 | 100.0 |
| | | | | γ | 0.754 | 100.0 |
| | | | | γ | 0.314 | 61.0 |
| | | | | γ | 0.527 | 45.0 |
| | | | | γ | 0.636 | 36.0 |
| | | | | γ | 0.629 | 31.0 |
| | | | | γ | 0.654 | 17.0 |
| | | | | γ | 0.814 | 13.0 |
| 51 | Sb-128m1 | 10.4 | m | β− | 0.000 | 78.0 |
| | | | | γ | 0.743 | 96.0 |
| | | | | γ | 0.754 | 96.0 |
| | | | | γ | 0.314 | 89.0 |
| 51 | Sb-129 | 4.4 | h | β− | 0.207 | 29.9 |
| | | | | β− | 0.165 | 25.5 |
| | | | | γ | 0.813 | 48.2 |
| | | | | γ | 0.915 | 23.3 |
| | | | | γ | 0.545 | 15.4 |
| | | | | γ | 1.031 | 15.1 |
| 51 | Sb-129m1 | 17.7 | m | β− | 0.904 | 63.0 |
| | | | | β− | 1.043 | 10.0 |
| | | | | γ | 0.760 | 85.0 |
| | | | | γ | 0.658 | 78.0 |
| | | | | γ | 0.434 | 62.0 |
| | | | | γ | 1.129 | 15.0 |
| | | | | γ | 0.723 | 14.2 |
| 51 | Sb-130 | 39.5 | m | β− | 1.154 | 18.0 |
| | | | | γ | 0.793 | 100.0 |
| | | | | γ | 0.840 | 100.0 |
| | | | | γ | 0.331 | 78.0 |
| | | | | γ | 0.182 | 65.0 |
| | | | | γ | 0.732 | 22.0 |
| | | | | γ | 0.935 | 19.0 |
| | | | | γ | 0.468 | 18.0 |
| 51 | Sb-131 | 23.0 | m | β− | 0.491 | 29.0 |
| | | | | β− | 1.046 | 12.0 |
| | | | | γ | 0.943 | 47.1 |
| | | | | γ | 0.933 | 26.4 |
| | | | | γ | 0.642 | 24.0 |
| 52 | Te-114 | 15.2 | m | β+ | 0.756 | 16.0 |
| | | | | EC | 0.686 | 25.0 |
| | | | | EC | 2.583 | 24.0 |
| 52 | Te-116 | 2.5 | h | EC | 1.456 | 93.7 |
| | | | | γ | 0.094 | 33.1 |
| 52 | Te-117 | 62.0 | m | β+ | 0.809 | 24.1 |
| | | | | EC | 2.829 | 28.2 |
| | | | | EC | 1.833 | 18.1 |
| | | | | EC | 1.249 | 11.4 |
| | | | | γ | 0.720 | 64.7 |
| | | | | γ | 1.716 | 15.9 |
| | | | | γ | 2.300 | 11.2 |
| 52 | Te-118 | 6.0 | d | EC | 0.278 | 100.0 |
| 52 | Te-119 | 16.1 | h | EC | 1.649 | 80.7 |
| | | | | γ | 0.644 | 84.1 |
| | | | | γ | 0.700 | 10.1 |
| 52 | Te-119m1 | 4.7 | d | EC | 1.188 | 67.0 |
| | | | | γ | 1.213 | 66.1 |
| | | | | γ | 0.154 | 66.0 |
| | | | | γ | 0.271 | 28.0 |
| 52 | Te-121 | 19.2 | d | EC | 0.481 | 82.0 |
| | | | | EC | 0.546 | 18.4 |
| | | | | γ | 0.573 | 80.4 |
| | | | | γ | 0.508 | 17.7 |
| 52 | Te-127 | 9.4 | h | β− | 0.228 | 98.8 |
| 52 | Te-129 | 69.6 | m | β− | 0.547 | 89.0 |
| | | | | γ | 0.028 | 16.3 |
| 52 | Te-129m1 | 33.6 | d | β− | 0.609 | 32.0 |
| 52 | Te-131 | 25.0 | m | β− | 0.818 | 59.3 |
| | | | | β− | 0.616 | 21.6 |
| | | | | β− | 0.382 | 10.0 |
| | | | | γ | 0.150 | 68.8 |
| | | | | γ | 0.452 | 18.2 |
| 52 | Te-131m1 | 33.3 | h | β− | 0.131 | 26.5 |
| | | | | β− | 0.160 | 11.9 |
| | | | | γ | 0.774 | 36.8 |
| | | | | γ | 0.852 | 19.9 |
| | | | | γ | 0.794 | 13.4 |
| | | | | γ | 1.125 | 11.0 |
| 52 | Te-132 | 3.2 | d | β− | 0.060 | 100.0 |
| | | | | γ | 0.228 | 88.0 |
| | | | | γ | 0.050 | 15.0 |
| 52 | Te-133 | 12.5 | m | β− | 0.880 | 28.2 |
| | | | | β− | 1.067 | 20.3 |
| | | | | β− | 0.605 | 13.0 |
| | | | | β− | 0.439 | 10.4 |
| | | | | γ | 0.312 | 62.4 |
| | | | | γ | 0.408 | 27.1 |
| | | | | γ | 1.333 | 10.7 |
| 52 | Te-133m1 | 55.4 | m | β− | 0.220 | 13.6 |
| | | | | γ | 0.913 | 44.0 |
| | | | | γ | 0.648 | 15.5 |
| | | | | γ | 0.864 | 12.5 |
| 52 | Te-134 | 41.8 | m | β− | 0.186 | 44.0 |
| | | | | β− | 0.214 | 42.0 |
| | | | | β− | 0.121 | 14.0 |
| | | | | γ | 0.767 | 29.5 |
| | | | | γ | 0.210 | 22.7 |
| | | | | γ | 0.278 | 21.2 |
| | | | | γ | 0.079 | 20.9 |
| | | | | γ | 0.435 | 18.9 |
| | | | | γ | 0.566 | 18.6 |
| | | | | γ | 0.181 | 18.3 |
| | | | | γ | 0.743 | 15.3 |
| 53 | I-118 | 13.7 | m | β+ | 2.504 | 32.8 |
| | | | | β+ | 2.769 | 16.7 |
| | | | | γ | 0.606 | 77.6 |
| | | | | γ | 0.545 | 10.0 |
| 53 | I-119 | 19.1 | m | β+ | 1.005 | 50.4 |
| | | | | EC | 3.163 | 33.3 |
| | | | | γ | 0.258 | 86.3 |
| 53 | I-120 | 81.6 | m | β+ | 1.845 | 29.3 |
| | | | | β+ | 2.099 | 19.0 |
| | | | | γ | 0.560 | 69.6 |
| | | | | γ | 1.523 | 10.9 |
| 53 | I-120m1 | 53.0 | m | β+ | 1.423 | 34.0 |
| | | | | EC | 2.813 | 11.0 |
| | | | | γ | 0.560 | 100.0 |
| | | | | γ | 0.601 | 87.0 |
| | | | | γ | 0.615 | 64.0 |
| | | | | γ | 1.346 | 19.0 |
| 53 | I-121 | 2.1 | h | β+ | 0.475 | 10.3 |
| | | | | EC | 2.080 | 76.2 |
| | | | | γ | 0.212 | 84.3 |
| 53 | I-123 | 13.2 | h | EC | 1.070 | 97.0 |
| | | | | γ | 0.159 | 83.3 |
| 53 | I-124 | 4.2 | d | β+ | 0.687 | 11.7 |
| | | | | β+ | 0.975 | 10.7 |
| | | | | EC | 2.557 | 25.2 |
| | | | | EC | 3.160 | 23.9 |
| | | | | EC | 0.866 | 11.6 |
| | | | | γ | 0.603 | 62.9 |
| | | | | γ | 1.691 | 11.2 |
| | | | | γ | 0.723 | 10.4 |
| 53 | I-126 | 12.9 | d | β− | 0.293 | 33.4 |
| | | | | β− | 0.462 | 10.3 |
| | | | | EC | 1.489 | 28.5 |
| | | | | EC | 2.155 | 18.7 |
| | | | | γ | 0.389 | 35.6 |
| | | | | γ | 0.666 | 32.9 |
| 53 | I-128 | 25.0 | m | β− | 0.833 | 80.0 |
| | | | | β− | 0.635 | 11.6 |
| | | | | γ | 0.443 | 12.6 |
| 53 | I-130 | 12.4 | h | β− | 0.347 | 48.0 |
| | | | | β− | 0.185 | 46.7 |
| | | | | γ | 0.536 | 99.0 |
| | | | | γ | 0.669 | 96.0 |
| | | | | γ | 0.740 | 82.0 |
| | | | | γ | 0.418 | 34.2 |
| | | | | γ | 1.157 | 11.3 |
| 53 | I-131 | 8.0 | d | β− | 0.192 | 89.6 |
| | | | | γ | 0.364 | 81.5 |
| 53 | I-132 | 2.3 | h | β− | 0.422 | 19.0 |
| | | | | β− | 0.842 | 19.0 |
| | | | | β− | 0.243 | 13.0 |
| | | | | β− | 0.608 | 12.3 |
| | | | | γ | 0.668 | 98.7 |
| | | | | γ | 0.773 | 75.6 |
| | | | | γ | 0.955 | 17.6 |
| | | | | γ | 0.523 | 16.0 |
| | | | | γ | 0.630 | 13.3 |
| 53 | I-132m1 | 1.4 | h | γ | 0.600 | 14.0 |
| | | | | γ | 0.773 | 14.0 |
| | | | | γ | 0.668 | 13.9 |
| 53 | I-133 | 20.8 | h | β− | 0.439 | 83.4 |
| | | | | γ | 0.530 | 87.0 |
| 53 | I-134 | 52.5 | m | β− | 0.474 | 30.4 |
| | | | | β− | 0.594 | 16.2 |
| | | | | β− | 0.980 | 12.6 |
| | | | | β− | 0.699 | 11.0 |
| | | | | γ | 0.847 | 96.0 |
| | | | | γ | 0.884 | 65.1 |
| | | | | γ | 1.073 | 14.9 |
| | | | | γ | 0.595 | 11.1 |
| | | | | γ | 0.622 | 10.6 |
| 53 | I-135 | 6.6 | h | β− | 0.499 | 23.6 |
| | | | | β− | 0.324 | 21.8 |
| | | | | γ | 1.260 | 28.7 |
| | | | | γ | 1.132 | 22.6 |
| 54 | Xe-120 | 40.0 | m | EC | 1.935 | 35.0 |
| | | | | EC | 0.994 | 15.9 |
| | | | | γ | 0.025 | 29.0 |
| 54 | Xe-121 | 40.1 | m | β+ | 1.245 | 20.0 |
| | | | | γ | 0.253 | 12.6 |
| | | | | γ | 0.133 | 11.0 |
| 54 | Xe-122 | 20.1 | h | EC | 0.725 | 83.0 |
| 54 | Xe-123 | 2.1 | h | β+ | 0.674 | 17.4 |
| | | | | EC | 2.546 | 43.9 |
| | | | | EC | 2.517 | 10.6 |
| | | | | γ | 0.149 | 48.9 |
| | | | | γ | 0.178 | 14.9 |
| 54 | Xe-125 | 16.9 | h | EC | 1.401 | 66.4 |
| | | | | EC | 1.456 | 25.3 |
| | | | | γ | 0.188 | 53.8 |
| | | | | γ | 0.243 | 30.0 |
| 54 | Xe-127 | 36.3 | d | EC | 0.459 | 53.0 |
| | | | | EC | 0.287 | 47.6 |
| | | | | γ | 0.203 | 68.7 |
| | | | | γ | 0.172 | 25.7 |
| | | | | γ | 0.375 | 17.3 |
| 54 | Xe-133 | 5.2 | d | β− | 0.101 | 98.5 |
| | | | | γ | 0.081 | 36.9 |
| 54 | Xe-133m1 | 2.2 | d | γ | 0.233 | 10.1 |
| 54 | Xe-135 | 9.1 | h | β− | 0.310 | 96.0 |
| | | | | γ | 0.250 | 90.0 |
| 54 | Xe-135m1 | 15.3 | m | γ | 0.527 | 80.6 |
| 54 | Xe-138 | 14.1 | m | β− | 0.300 | 35.3 |
| | | | | β− | 0.988 | 21.7 |
| | | | | β− | 1.005 | 15.0 |
| | | | | β− | 1.188 | 11.0 |
| | | | | β− | 0.208 | 10.3 |
| | | | | γ | 0.258 | 34.1 |
| | | | | γ | 0.435 | 22.0 |
| | | | | γ | 1.768 | 18.1 |
| | | | | γ | 2.016 | 13.3 |
| 55 | Cs-125 | 46.7 | m | β+ | 0.936 | 27.0 |
| | | | | EC | 3.104 | 26.0 |
| | | | | EC | 2.579 | 21.0 |
| | | | | γ | 0.526 | 24.6 |
| 55 | Cs-127 | 6.3 | h | EC | 1.669 | 66.8 |
| | | | | EC | 2.081 | 11.1 |
| | | | | γ | 0.412 | 62.9 |
| | | | | γ | 0.125 | 11.4 |
| 55 | Cs-129 | 32.1 | h | EC | 0.786 | 55.0 |
| | | | | EC | 1.197 | 34.0 |
| | | | | γ | 0.372 | 30.6 |
| | | | | γ | 0.411 | 22.3 |
| 55 | Cs-130 | 29.2 | m | β+ | 0.879 | 43.0 |
| | | | | EC | 2.979 | 51.5 |
| | | | | γ | 0.080 | 34.6 |
| 55 | Cs-131 | 9.7 | d | EC | 0.355 | 100.0 |
| 55 | Cs-132 | 6.5 | d | EC | 1.457 | 95.5 |
| | | | | γ | 0.668 | 97.6 |
| 55 | Cs-134 | 2.9 | h | γ | 0.128 | 12.6 |
| 55 | Cs-135 | 53.0 | m | γ | 0.787 | 99.7 |
| | | | | γ | 0.846 | 96.0 |
| 55 | Cs-136 | 13.0 | d | β− | 0.099 | 80.8 |
| | | | | β− | 0.121 | 13.6 |
| | | | | γ | 0.819 | 99.7 |
| | | | | γ | 1.048 | 80.0 |
| | | | | γ | 0.341 | 46.8 |
| | | | | γ | 1.235 | 20.0 |
| | | | | γ | 0.177 | 13.7 |
| | | | | γ | 0.274 | 12.7 |
| 55 | Cs-138 | 32.5 | m | β− | 1.200 | 44.0 |
| | | | | β− | 1.454 | 13.7 |
| | | | | β− | 1.305 | 12.9 |
| | | | | γ | 1.436 | 76.3 |
| | | | | γ | 0.463 | 30.8 |
| | | | | γ | 1.010 | 29.8 |
| | | | | γ | 2.218 | 15.2 |
| | | | | γ | 0.547 | 10.8 |
| 56 | Ba-124 | 11.0 | m | EC | 2.472 | 14.4 |
| | | | | EC | 1.425 | 13.1 |
| | | | | EC | 2.642 | 12.0 |
| | | | | γ | 0.170 | 22.0 |
| | | | | γ | 0.189 | 12.9 |
| 56 | Ba-126 | 100.0 | m | EC | 1.673 | 30.0 |
| | | | | EC | 1.439 | 16.8 |
| | | | | γ | 0.234 | 19.6 |
| 56 | Ba-127 | 12.7 | m | β+ | 1.083 | 36.1 |
| | | | | β+ | 1.000 | 15.0 |
| | | | | EC | 3.424 | 25.1 |
| | | | | EC | 3.243 | 14.0 |
| | | | | γ | 0.181 | 12.5 |
| 56 | Ba-128 | 2.4 | d | EC | 0.553 | 84.0 |
| | | | | EC | 0.280 | 15.3 |
| | | | | γ | 0.273 | 14.5 |
| 56 | Ba-129 | 2.2 | h | β+ | 0.635 | 13.0 |
| | | | | EC | 2.436 | 47.0 |
| | | | | EC | 2.215 | 20.4 |
| | | | | γ | 0.214 | 13.4 |
| 56 | Ba-129m1 | 2.1 | h | EC | 0.796 | 59.5 |
| | | | | EC | 0.632 | 15.0 |
| 56 | Ba-131 | 11.5 | d | EC | 0.756 | 52.7 |
| | | | | EC | 1.160 | 21.7 |
| | | | | EC | 1.003 | 20.2 |
| | | | | γ | 0.496 | 48.0 |
| | | | | γ | 0.124 | 29.8 |
| | | | | γ | 0.216 | 20.4 |
| | | | | γ | 0.373 | 14.4 |
| 56 | Ba-131m1 | 14.6 | m | γ | 0.108 | 55.4 |
| 56 | Ba-133 | 38.9 | h | γ | 0.276 | 17.7 |
| 56 | Ba-135 | 28.7 | h | γ | 0.268 | 16.0 |
| 56 | Ba-139 | 82.9 | m | β− | 0.916 | 70.0 |
| | | | | β− | 0.841 | 29.7 |
| | | | | γ | 0.166 | 23.7 |
| 56 | Ba-140 | 12.8 | d | β− | 0.345 | 40.0 |
| | | | | β− | 0.141 | 24.7 |
| | | | | β− | 0.362 | 20.0 |
| | | | | γ | 0.537 | 24.4 |
| | | | | γ | 0.030 | 14.1 |
| 56 | Ba-141 | 18.3 | m | β− | 1.025 | 24.4 |
| | | | | β− | 1.108 | 18.1 |
| | | | | β− | 0.897 | 12.5 |
| | | | | γ | 0.190 | 45.5 |
| | | | | γ | 0.304 | 25.2 |
| | | | | γ | 0.277 | 23.2 |
| | | | | γ | 0.344 | 14.3 |
| 56 | Ba-142 | 10.6 | m | β− | 0.346 | 46.0 |
| | | | | β− | 0.398 | 22.0 |
| | | | | β− | 0.246 | 15.4 |
| | | | | γ | 0.255 | 20.6 |
| | | | | γ | 1.204 | 14.3 |
| | | | | γ | 0.895 | 13.9 |
| | | | | γ | 0.232 | 12.2 |
| | | | | γ | 1.079 | 11.5 |
| | | | | γ | 0.949 | 10.7 |
| 57 | La-129 | 11.6 | m | β+ | 1.100 | 15.0 |
| | | | | β+ | 1.228 | 11.0 |
| | | | | EC | 3.460 | 10.9 |
| | | | | γ | 0.279 | 24.7 |
| | | | | γ | 0.111 | 16.9 |
| 57 | La-131 | 59.0 | m | EC | 2.389 | 24.6 |
| | | | | EC | 2.550 | 15.9 |
| | | | | γ | 0.108 | 25.0 |
| | | | | γ | 0.418 | 17.9 |
| | | | | γ | 0.365 | 16.9 |
| | | | | γ | 0.285 | 12.4 |
| 57 | La-132 | 24.3 | m | γ | 0.136 | 49.0 |
| | | | | γ | 0.465 | 21.6 |
| | | | | γ | 0.663 | 11.2 |
| 57 | La-133 | 3.9 | h | EC | 2.047 | 84.0 |
| 57 | La-135 | 19.5 | h | EC | 1.200 | 98.1 |
| 57 | La-140 | 1.7 | d | β− | 0.487 | 43.9 |
| | | | | β− | 0.629 | 20.2 |
| | | | | β− | 0.441 | 11.1 |
| | | | | γ | 1.596 | 95.4 |
| | | | | γ | 0.487 | 45.5 |
| | | | | γ | 0.816 | 23.3 |
| | | | | γ | 0.329 | 20.3 |
| 57 | La-141 | 3.9 | h | β− | 0.999 | 98.1 |
| 57 | La-142 | 91.1 | m | β− | 0.820 | 17.8 |
| | | | | β− | 0.755 | 17.3 |
| | | | | β− | 1.904 | 16.5 |
| | | | | γ | 0.641 | 47.4 |
| | | | | γ | 2.398 | 13.3 |
| | | | | γ | 2.543 | 10.0 |
| 57 | La-143 | 14.2 | m | β− | 1.409 | 52.0 |
| | | | | β− | 1.410 | 32.0 |
| 58 | Ce-130 | 22.9 | m | EC | 2.080 | 39.0 |
| | | | | EC | 1.014 | 11.0 |
| | | | | γ | 0.131 | 37.0 |
| 58 | Ce-132 | 3.5 | h | EC | 1.088 | 77.1 |
| | | | | γ | 0.182 | 77.4 |
| | | | | γ | 0.155 | 10.5 |
| 58 | Ce-133 | 97.0 | m | β+ | 0.843 | 19.0 |
| | | | | β+ | 0.878 | 18.0 |
| | | | | EC | 2.897 | 34.0 |
| | | | | EC | 2.974 | 29.0 |
| | | | | γ | 0.097 | 45.0 |
| | | | | γ | 0.077 | 15.9 |
| | | | | γ | 0.558 | 11.4 |
| 58 | Ce-133m1 | 5.1 | h | EC | 1.072 | 14.7 |
| | | | | γ | 0.477 | 39.3 |
| | | | | γ | 0.510 | 20.8 |
| | | | | γ | 0.058 | 19.3 |
| | | | | γ | 0.131 | 18.0 |
| 58 | Ce-134 | 3.2 | d | EC | 0.380 | 98.9 |
| 58 | Ce-135 | 17.7 | h | EC | 1.154 | 33.7 |
| | | | | EC | 1.242 | 30.1 |
| | | | | EC | 1.727 | 14.4 |
| | | | | γ | 0.266 | 41.8 |
| | | | | γ | 0.300 | 23.5 |
| | | | | γ | 0.607 | 18.8 |
| | | | | γ | 0.518 | 13.6 |
| | | | | γ | 0.784 | 10.6 |
| | | | | γ | 0.572 | 10.4 |
| 58 | Ce-137 | 9.0 | h | EC | 1.211 | 97.8 |
| 58 | Ce-137m1 | 34.4 | h | γ | 0.254 | 11.1 |
| 58 | Ce-141 | 32.5 | d | β− | 0.130 | 69.7 |
| | | | | β− | 0.181 | 30.3 |
| | | | | γ | 0.145 | 48.4 |
| 58 | Ce-143 | 33.0 | h | β− | 0.387 | 48.2 |
| | | | | β− | 0.511 | 35.0 |
| | | | | β− | 0.240 | 13.4 |
| | | | | γ | 0.293 | 42.8 |
| | | | | γ | 0.057 | 11.7 |
| 58 | Ce-146 | 13.5 | m | β− | 0.224 | 94.3 |
| | | | | γ | 0.317 | 55.3 |
| | | | | γ | 0.218 | 19.5 |
| 59 | Pr-134 | 17.0 | m | β+ | 2.248 | 32.0 |
| | | | | β+ | 1.984 | 20.0 |
| | | | | β+ | 1.245 | 11.0 |
| | | | | γ | 0.409 | 87.1 |
| | | | | γ | 0.556 | 13.1 |
| | | | | γ | 0.966 | 13.1 |
| | | | | γ | 2.136 | 10.7 |
| 59 | Pr-134m1 | 11.0 | m | β+ | 1.331 | 24.0 |
| | | | | β+ | 1.330 | 15.0 |
| | | | | EC | 3.961 | 12.0 |
| | | | | γ | 0.409 | 89.3 |
| | | | | γ | 0.640 | 59.7 |
| | | | | γ | 0.215 | 21.4 |
| | | | | γ | 0.332 | 19.0 |
| | | | | γ | 0.334 | 19.0 |
| | | | | γ | 0.974 | 15.3 |
| | | | | γ | 0.979 | 12.5 |
| | | | | γ | 0.678 | 11.7 |
| | | | | γ | 0.966 | 11.0 |
| | | | | γ | 0.384 | 10.7 |
| | | | | γ | 0.556 | 10.7 |
| | | | | γ | 1.125 | 10.7 |
| 59 | Pr-136 | 13.1 | m | β+ | 1.384 | 40.6 |
| | | | | EC | 4.076 | 17.7 |
| | | | | γ | 0.552 | 75.5 |
| | | | | γ | 0.540 | 52.1 |
| | | | | γ | 1.092 | 18.4 |
| 59 | Pr-137 | 1.3 | h | β+ | 0.755 | 24.8 |
| | | | | EC | 2.702 | 68.7 |
| 59 | Pr-138m1 | 2.0 | h | β+ | 0.742 | 23.0 |
| | | | | EC | 2.672 | 68.0 |
| | | | | γ | 1.038 | 101.0 |
| | | | | γ | 0.789 | 100.0 |
| | | | | γ | 0.303 | 80.0 |
| 59 | Pr-139 | 4.4 | h | EC | 2.129 | 90.5 |
| 59 | Pr-142 | 19.1 | h | β− | 0.834 | 96.3 |
| 59 | Pr-143 | 13.6 | d | β− | 0.315 | 100.0 |
| 59 | Pr-144 | 17.3 | m | β− | 1.222 | 97.9 |
| 59 | Pr-145 | 6.0 | h | β− | 0.683 | 97.6 |
| 59 | Pr-146 | 24.1 | m | β− | 1.776 | 45.0 |
| | | | | β− | 0.889 | 28.7 |
| | | | | β− | 1.588 | 13.0 |
| | | | | γ | 0.454 | 46.0 |
| | | | | γ | 1.525 | 15.0 |
| 59 | Pr-147 | 13.4 | m | β− | 0.727 | 23.4 |
| | | | | β− | 0.738 | 21.3 |
| | | | | β− | 0.941 | 12.6 |
| | | | | β− | 0.502 | 10.2 |
| | | | | γ | 0.315 | 17.5 |
| | | | | γ | 0.641 | 15.6 |
| | | | | γ | 0.578 | 13.7 |
| 60 | Nd-135 | 12.4 | m | β+ | 1.518 | 14.0 |
| | | | | γ | 0.204 | 49.6 |
| | | | | γ | 0.041 | 22.0 |
| | | | | γ | 0.441 | 14.3 |
| 60 | Nd-136 | 50.7 | m | EC | 1.992 | 70.4 |
| | | | | EC | 1.566 | 12.2 |
| | | | | γ | 0.109 | 32.0 |
| | | | | γ | 0.040 | 19.2 |
| | | | | γ | 0.575 | 10.6 |
| 60 | Nd-137 | 38.5 | m | β+ | 1.128 | 13.0 |
| | | | | EC | 3.016 | 13.0 |
| | | | | EC | 3.422 | 11.0 |
| | | | | γ | 0.076 | 17.3 |
| | | | | γ | 0.581 | 13.0 |
| | | | | γ | 0.307 | 10.1 |
| 60 | Nd-138 | 5.0 | h | EC | 1.116 | 95.9 |
| 60 | Nd-139 | 29.7 | m | β+ | 0.803 | 24.0 |
| | | | | EC | 2.806 | 61.0 |
| 60 | Nd-139m1 | 5.5 | h | EC | 1.203 | 33.8 |
| | | | | EC | 1.110 | 12.5 |
| | | | | γ | 0.114 | 40.0 |
| | | | | γ | 0.738 | 35.1 |
| | | | | γ | 0.708 | 26.3 |
| | | | | γ | 0.982 | 26.3 |
| | | | | γ | 0.828 | 10.3 |
| 60 | Nd-140 | 3.4 | d | EC | 0.429 | 100.0 |
| 60 | Nd-141 | 2.5 | h | EC | 1.824 | 95.6 |
| 60 | Nd-147 | 11.0 | d | β− | 0.264 | 80.2 |
| | | | | β− | 0.106 | 15.4 |
| | | | | γ | 0.091 | 28.1 |
| | | | | γ | 0.531 | 13.4 |
| 60 | Nd-149 | 1.7 | h | β− | 0.541 | 24.7 |
| | | | | β− | 0.403 | 21.5 |
| | | | | β− | 0.355 | 19.1 |
| | | | | β− | 0.515 | 17.5 |
| | | | | γ | 0.211 | 25.9 |
| | | | | γ | 0.114 | 19.2 |
| | | | | γ | 0.270 | 10.7 |
| 60 | Nd-151 | 12.4 | m | β− | 0.400 | 17.9 |
| | | | | β− | 0.966 | 14.6 |
| | | | | γ | 0.117 | 39.0 |
| | | | | γ | 0.256 | 14.8 |
| | | | | γ | 1.181 | 13.3 |
| 60 | Nd-152 | 11.4 | m | β− | 0.373 | 52.0 |
| | | | | β− | 0.384 | 52.0 |
| | | | | β− | 0.266 | 47.0 |
| | | | | γ | 0.279 | 26.0 |
| | | | | γ | 0.250 | 16.4 |
| 61 | Pm-141 | 20.9 | m | β+ | 1.197 | 49.3 |
| | | | | EC | 3.730 | 39.4 |
| 61 | Pm-148 | 5.4 | d | β− | 0.978 | 55.5 |
| | | | | β− | 0.343 | 33.4 |
| | | | | γ | 1.465 | 22.2 |
| | | | | γ | 0.550 | 22.0 |
| | | | | γ | 0.915 | 11.5 |
| 61 | Pm-148m1 | 41.3 | d | β− | 0.123 | 54.0 |
| | | | | β− | 0.225 | 21.9 |
| | | | | β− | 0.156 | 18.7 |
| | | | | γ | 0.550 | 94.9 |
| | | | | γ | 0.630 | 89.0 |
| | | | | γ | 0.726 | 32.8 |
| | | | | γ | 1.014 | 20.3 |
| | | | | γ | 0.414 | 18.7 |
| | | | | γ | 0.915 | 17.2 |
| | | | | γ | 0.288 | 12.6 |
| | | | | γ | 0.600 | 12.5 |
| 61 | Pm-149 | 53.1 | h | β− | 0.369 | 95.9 |
| 61 | Pm-150 | 2.7 | h | β− | 0.895 | 25.9 |
| | | | | β− | 0.677 | 19.4 |
| | | | | β− | 0.499 | 17.5 |
| | | | | β− | 1.427 | 10.0 |
| | | | | γ | 0.334 | 68.0 |
| | | | | γ | 1.325 | 17.5 |
| | | | | γ | 1.166 | 15.8 |
| | | | | γ | 0.832 | 11.9 |
| 61 | Pm-151 | 28.4 | h | β− | 0.278 | 42.0 |
| | | | | β− | 0.415 | 10.0 |
| | | | | β− | 0.417 | 10.0 |
| | | | | γ | 0.340 | 22.5 |
| 62 | Sm-140 | 14.8 | m | β+ | 0.779 | 18.0 |
| | | | | EC | 2.758 | 57.0 |
| 62 | Sm-141 | 10.2 | m | β+ | 1.434 | 21.2 |
| | | | | β+ | 1.419 | 20.1 |
| | | | | EC | 4.200 | 10.8 |
| | | | | EC | 4.120 | 10.6 |
| | | | | γ | 0.404 | 42.5 |
| | | | | γ | 0.438 | 37.7 |
| 62 | Sm-141m1 | 22.6 | m | EC | 2.620 | 18.2 |
| | | | | EC | 2.673 | 12.8 |
| | | | | EC | 2.690 | 11.2 |
| | | | | γ | 0.197 | 74.0 |
| | | | | γ | 0.432 | 40.4 |
| | | | | γ | 0.777 | 20.3 |
| | | | | γ | 1.786 | 10.9 |
| 62 | Sm-142 | 72.5 | m | EC | 2.050 | 92.5 |
| 62 | Sm-153 | 46.3 | h | β− | 0.225 | 49.4 |
| | | | | β− | 0.200 | 31.3 |
| | | | | β− | 0.264 | 18.4 |
| | | | | γ | 0.103 | 29.3 |
| 62 | Sm-155 | 22.3 | m | β− | 0.557 | 92.8 |
| | | | | γ | 0.104 | 74.6 |
| 62 | Sm-156 | 9.4 | h | β− | 0.223 | 48.0 |
| | | | | β− | 0.128 | 44.0 |
| | | | | γ | 0.088 | 24.0 |
| | | | | γ | 0.204 | 21.0 |
| | | | | γ | 0.166 | 13.0 |
| 63 | Eu-145 | 5.9 | d | EC | 1.766 | 43.0 |
| | | | | EC | 1.112 | 19.3 |
| | | | | EC | 1.001 | 16.4 |
| | | | | γ | 0.894 | 66.0 |
| | | | | γ | 0.654 | 15.0 |
| | | | | γ | 1.659 | 14.9 |
| 63 | Eu-146 | 4.6 | d | EC | 2.488 | 14.5 |
| | | | | EC | 2.488 | 10.5 |
| | | | | γ | 0.747 | 98.0 |
| | | | | γ | 0.634 | 44.8 |
| | | | | γ | 0.633 | 35.7 |
| | | | | γ | 0.665 | 10.3 |
| 63 | Eu-147 | 24.1 | d | EC | 1.524 | 24.9 |
| | | | | EC | 1.600 | 20.6 |
| | | | | EC | 0.923 | 18.7 |
| | | | | EC | 1.722 | 16.9 |
| | | | | γ | 0.197 | 24.4 |
| | | | | γ | 0.121 | 21.2 |
| 63 | Eu-150 | 12.8 | h | β− | 0.345 | 89.0 |
| 63 | Eu-152m1 | 9.3 | h | β− | 0.704 | 70.0 |
| | | | | EC | 0.957 | 26.0 |
| | | | | γ | 0.842 | 14.2 |
| | | | | γ | 0.963 | 11.6 |
| 63 | Eu-152m2 | 96.0 | m | γ | 0.090 | 69.7 |
| 63 | Eu-154 | 46.3 | m | γ | 0.068 | 37.0 |
| | | | | γ | 0.101 | 27.0 |
| | | | | γ | 0.036 | 13.0 |
| 63 | Eu-156 | 15.2 | d | β− | 0.965 | 32.0 |
| | | | | β− | 0.146 | 29.0 |
| | | | | β− | 0.074 | 10.3 |
| 63 | Eu-157 | 15.2 | h | β− | 0.462 | 49.0 |
| | | | | β− | 0.296 | 22.0 |
| | | | | β− | 0.312 | 15.0 |
| | | | | γ | 0.064 | 23.0 |
| | | | | γ | 0.411 | 17.8 |
| | | | | γ | 0.371 | 11.2 |
| 63 | Eu-158 | 45.9 | m | β− | 0.946 | 25.0 |
| | | | | β− | 0.967 | 21.3 |
| | | | | γ | 0.944 | 30.0 |
| | | | | γ | 0.977 | 16.3 |
| | | | | γ | 0.898 | 12.4 |
| | | | | γ | 0.079 | 10.5 |
| 63 | Eu-159 | 18.1 | m | β− | 0.927 | 37.0 |
| | | | | β− | 0.963 | 19.0 |
| | | | | β− | 0.891 | 14.0 |
| | | | | γ | 0.068 | 19.0 |
| 64 | Gd-145 | 23.0 | m | β+ | 1.024 | 13.0 |
| | | | | β+ | 0.969 | 11.8 |
| | | | | EC | 3.190 | 23.0 |
| | | | | EC | 3.313 | 21.3 |
| | | | | γ | 1.758 | 34.2 |
| | | | | γ | 1.881 | 32.6 |
| 64 | Gd-146 | 48.3 | d | EC | 0.647 | 72.1 |
| | | | | EC | 0.802 | 26.5 |
| | | | | γ | 0.155 | 46.9 |
| | | | | γ | 0.115 | 44.3 |
| | | | | γ | 0.116 | 44.3 |
| 64 | Gd-147 | 38.1 | h | EC | 0.633 | 35.6 |
| | | | | EC | 1.192 | 19.0 |
| | | | | γ | 0.229 | 58.0 |
| | | | | γ | 0.396 | 31.3 |
| | | | | γ | 0.929 | 18.4 |
| | | | | γ | 0.370 | 15.7 |
| | | | | γ | 0.766 | 10.4 |
| 64 | Gd-149 | 9.3 | d | EC | 1.163 | 34.4 |
| | | | | EC | 0.518 | 33.9 |
| | | | | EC | 0.374 | 14.0 |
| | | | | γ | 0.150 | 48.0 |
| | | | | γ | 0.299 | 28.6 |
| | | | | γ | 0.347 | 23.9 |
| 64 | Gd-159 | 18.5 | h | β− | 0.327 | 58.6 |
| | | | | β− | 0.304 | 28.8 |
| | | | | β− | 0.189 | 12.2 |
| | | | | γ | 0.364 | 11.8 |
| 65 | Tb-147 | 1.6 | h | EC | 2.764 | 34.9 |
| | | | | γ | 1.153 | 90.2 |
| | | | | γ | 0.695 | 37.3 |
| | | | | γ | 0.140 | 30.7 |
| 65 | Tb-148 | 60.0 | m | β+ | 1.791 | 21.8 |
| | | | | γ | 0.784 | 84.0 |
| | | | | γ | 0.489 | 19.7 |
| | | | | γ | 1.079 | 11.4 |
| | | | | γ | 0.632 | 10.6 |
| 65 | Tb-149 | 4.1 | h | α | 3.967 | 16.7 |
| | | | | EC | 2.431 | 31.5 |
| | | | | γ | 0.352 | 29.4 |
| | | | | γ | 0.165 | 26.4 |
| | | | | γ | 0.389 | 18.4 |
| | | | | γ | 0.652 | 16.2 |
| | | | | γ | 0.853 | 15.5 |
| | | | | γ | 0.817 | 11.6 |
| 65 | Tb-150 | 3.5 | h | β+ | 1.358 | 14.0 |
| | | | | EC | 4.020 | 11.0 |
| | | | | γ | 0.638 | 72.0 |
| | | | | γ | 0.511 | 45.0 |
| | | | | γ | 0.496 | 14.6 |
| 65 | Tb-151 | 17.6 | h | EC | 1.726 | 48.4 |
| | | | | EC | 1.373 | 18.7 |
| | | | | γ | 0.287 | 28.3 |
| | | | | γ | 0.252 | 26.3 |
| | | | | γ | 0.108 | 24.3 |
| | | | | γ | 0.587 | 15.6 |
| | | | | γ | 0.479 | 15.4 |
| | | | | γ | 0.180 | 11.5 |
| | | | | γ | 0.395 | 10.8 |
| | | | | γ | 0.444 | 10.8 |
| | | | | γ | 0.617 | 10.4 |
| 65 | Tb-152 | 17.5 | h | EC | 3.990 | 17.0 |
| | | | | γ | 0.344 | 63.5 |
| 65 | Tb-153 | 2.3 | d | EC | 1.358 | 48.0 |
| | | | | EC | 1.528 | 15.0 |
| | | | | γ | 0.212 | 31.0 |
| 65 | Tb-154 | 21.5 | h | EC | 1.430 | 33.0 |
| | | | | EC | 1.363 | 18.7 |
| | | | | γ | 0.123 | 26.0 |
| | | | | γ | 1.274 | 10.5 |
| 65 | Tb-154m1 | 9.4 | h | γ | 0.123 | 30.0 |
| | | | | γ | 0.248 | 22.0 |
| | | | | γ | 0.540 | 20.0 |
| | | | | γ | 0.649 | 10.9 |
| | | | | γ | 1.005 | 10.9 |
| 65 | Tb-154m2 | 22.7 | h | γ | 0.248 | 79.0 |
| | | | | γ | 0.347 | 69.0 |
| | | | | γ | 1.420 | 46.0 |
| | | | | γ | 0.123 | 43.0 |
| | | | | γ | 0.226 | 27.0 |
| | | | | γ | 0.427 | 17.3 |
| | | | | γ | 0.993 | 16.2 |
| 65 | Tb-155 | 5.3 | d | EC | 0.718 | 37.5 |
| | | | | EC | 0.556 | 18.0 |
| | | | | γ | 0.087 | 32.0 |
| | | | | γ | 0.105 | 25.1 |
| 65 | Tb-156 | 5.4 | d | EC | 0.399 | 75.0 |
| | | | | EC | 0.510 | 12.0 |
| | | | | γ | 0.534 | 67.0 |
| | | | | γ | 0.199 | 41.0 |
| | | | | γ | 1.222 | 31.0 |
| | | | | γ | 0.089 | 18.0 |
| | | | | γ | 0.356 | 13.6 |
| | | | | γ | 1.422 | 12.2 |
| | | | | γ | 1.065 | 10.8 |
| | | | | γ | 1.154 | 10.4 |
| 65 | Tb-156m1 | 24.4 | h | γ | 0.050 | 74.1 |
| 65 | Tb-161 | 6.9 | d | β− | 0.157 | 65.0 |
| | | | | β− | 0.138 | 25.7 |
| | | | | γ | 0.026 | 23.2 |
| | | | | γ | 0.049 | 17.0 |
| | | | | γ | 0.075 | 10.2 |
| 65 | Tb-163 | 19.5 | m | β− | 0.299 | 34.8 |
| | | | | β− | 0.279 | 15.4 |
| | | | | β− | 0.346 | 11.5 |
| | | | | β− | 0.357 | 11.4 |
| | | | | γ | 0.351 | 26.0 |
| | | | | γ | 0.390 | 24.0 |
| | | | | γ | 0.495 | 22.5 |
| | | | | γ | 0.422 | 11.5 |
| 66 | Dy-151 | 17.9 | m | γ | 0.386 | 19.4 |
| | | | | γ | 0.049 | 18.0 |
| | | | | γ | 0.546 | 14.3 |
| | | | | γ | 0.176 | 10.6 |
| 66 | Dy-152 | 2.4 | h | EC | 0.343 | 99.9 |
| | | | | γ | 0.257 | 97.5 |
| 66 | Dy-153 | 6.4 | h | EC | 1.908 | 26.6 |
| | | | | γ | 0.081 | 11.1 |
| | | | | γ | 0.214 | 10.9 |
| | | | | γ | 0.100 | 10.5 |
| 66 | Dy-155 | 9.9 | h | EC | 1.868 | 64.9 |
| | | | | EC | 0.939 | 10.8 |
| | | | | γ | 0.227 | 68.4 |
| 66 | Dy-157 | 8.1 | h | EC | 1.013 | 96.0 |
| | | | | γ | 0.326 | 93.0 |
| 66 | Dy-165 | 2.3 | h | β− | 0.454 | 83.0 |
| | | | | β− | 0.415 | 15.0 |
| 66 | Dy-166 | 81.6 | h | β− | 0.118 | 97.0 |
| | | | | γ | 0.082 | 13.8 |
| 67 | Ho-154 | 11.8 | m | β+ | 2.007 | 12.1 |
| | | | | γ | 0.335 | 84.4 |
| | | | | γ | 0.412 | 15.2 |
| | | | | γ | 0.873 | 12.2 |
| | | | | γ | 0.569 | 11.1 |
| | | | | γ | 0.571 | 11.1 |
| 67 | Ho-155 | 48.0 | m | γ | 0.240 | 12.5 |
| 67 | Ho-156 | 56.0 | m | γ | 0.266 | 66.0 |
| | | | | γ | 0.138 | 52.0 |
| | | | | γ | 0.366 | 14.5 |
| 67 | Ho-157 | 12.6 | m | EC | 2.202 | 53.0 |
| | | | | EC | 1.696 | 12.0 |
| | | | | γ | 0.280 | 23.1 |
| | | | | γ | 0.341 | 17.8 |
| 67 | Ho-158m2 | 21.3 | m | EC | 1.872 | 93.0 |
| 67 | Ho-159 | 33.1 | m | EC | 1.528 | 70.0 |
| | | | | EC | 1.442 | 13.8 |
| | | | | γ | 0.121 | 36.2 |
| | | | | γ | 0.132 | 23.6 |
| | | | | γ | 0.310 | 17.2 |
| | | | | γ | 0.253 | 13.7 |
| 67 | Ho-161 | 2.5 | h | EC | 0.833 | 76.0 |
| | | | | EC | 0.755 | 19.0 |
| | | | | γ | 0.026 | 27.0 |
| 67 | Ho-162 | 15.0 | m | EC | 2.090 | 50.0 |
| | | | | EC | 2.170 | 40.0 |
| 67 | Ho-162m1 | 67.0 | m | EC | 0.760 | 13.9 |
| | | | | γ | 0.185 | 23.9 |
| | | | | γ | 1.220 | 23.7 |
| | | | | γ | 0.937 | 10.4 |
| | | | | γ | 0.283 | 10.3 |
| 67 | Ho-164 | 28.8 | m | β− | 0.322 | 28.0 |
| | | | | β− | 0.286 | 12.0 |
| | | | | EC | 0.961 | 41.0 |
| | | | | EC | 0.888 | 19.0 |
| 67 | Ho-164m1 | 36.6 | m | γ | 0.037 | 11.4 |
| 67 | Ho-166 | 26.8 | h | β− | 0.651 | 49.9 |
| | | | | β− | 0.694 | 48.8 |
| 67 | Ho-167 | 3.1 | h | β− | 0.097 | 43.0 |
| | | | | β− | 0.208 | 21.0 |
| | | | | β− | 0.309 | 15.0 |
| | | | | β− | 0.340 | 15.0 |
| | | | | γ | 0.347 | 57.0 |
| | | | | γ | 0.321 | 24.0 |
| 68 | Er-156 | 19.5 | m | EC | 1.142 | 52.0 |
| | | | | EC | 1.208 | 23.0 |
| | | | | EC | 1.169 | 18.0 |
| | | | | γ | 0.035 | 26.0 |
| 68 | Er-157 | 18.7 | m | EC | 3.266 | 17.0 |
| | | | | EC | 3.049 | 11.0 |
| | | | | γ | 0.053 | 24.1 |
| | | | | γ | 0.391 | 14.2 |
| | | | | γ | 0.121 | 10.1 |
| 68 | Er-158 | 2.3 | h | EC | 0.733 | 76.0 |
| | | | | γ | 0.072 | 10.8 |
| 68 | Er-159 | 36.0 | m | EC | 2.144 | 32.0 |
| | | | | EC | 2.119 | 25.0 |
| | | | | γ | 0.625 | 33.0 |
| | | | | γ | 0.649 | 23.0 |
| 68 | Er-160 | 28.6 | h | EC | 0.263 | 100.0 |
| 68 | Er-161 | 3.2 | h | EC | 1.167 | 66.0 |
| | | | | γ | 0.827 | 64.0 |
| | | | | γ | 0.211 | 12.2 |
| 68 | Er-163 | 75.0 | m | EC | 1.210 | 99.9 |
| 68 | Er-165 | 10.4 | h | EC | 0.371 | 100.0 |
| 68 | Er-169 | 9.4 | d | β− | 0.101 | 55.0 |
| | | | | β− | 0.098 | 45.0 |
| 68 | Er-171 | 7.5 | h | β− | 0.363 | 94.0 |
| | | | | γ | 0.308 | 64.0 |
| | | | | γ | 0.296 | 28.9 |
| | | | | γ | 0.112 | 20.5 |
| 68 | Er-172 | 49.3 | h | β− | 0.079 | 46.4 |
| | | | | β− | 0.102 | 46.4 |
| | | | | γ | 0.610 | 44.2 |
| | | | | γ | 0.407 | 42.1 |
| 69 | Tm-161 | 30.2 | m | γ | 0.046 | 25.0 |
| | | | | γ | 1.648 | 19.5 |
| 69 | Tm-162 | 21.7 | m | γ | 0.102 | 17.5 |
| 69 | Tm-163 | 1.8 | h | EC | 0.900 | 16.9 |
| | | | | EC | 0.637 | 12.9 |
| | | | | γ | 0.104 | 18.6 |
| | | | | γ | 0.069 | 11.6 |
| | | | | γ | 0.241 | 10.9 |
| 69 | Tm-165 | 30.1 | h | EC | 1.295 | 35.9 |
| | | | | EC | 1.236 | 12.2 |
| | | | | EC | 0.739 | 10.2 |
| | | | | γ | 0.243 | 35.5 |
| | | | | γ | 0.047 | 16.9 |
| | | | | γ | 0.297 | 12.7 |
| 69 | Tm-166 | 7.7 | h | EC | 0.905 | 59.0 |
| | | | | EC | 0.878 | 16.2 |
| | | | | γ | 0.779 | 19.1 |
| | | | | γ | 2.052 | 17.4 |
| | | | | γ | 0.184 | 16.2 |
| | | | | γ | 1.274 | 15.0 |
| | | | | γ | 0.081 | 11.5 |
| | | | | γ | 0.705 | 11.1 |
| | | | | γ | 0.786 | 10.0 |
| 69 | Tm-167 | 9.3 | d | EC | 0.541 | 69.0 |
| | | | | EC | 0.484 | 29.0 |
| | | | | γ | 0.208 | 42.0 |
| 69 | Tm-172 | 63.6 | h | β− | 0.668 | 36.0 |
| | | | | β− | 0.691 | 29.0 |
| | | | | β− | 0.121 | 10.1 |
| | | | | β− | 0.076 | 10.0 |
| 69 | Tm-173 | 8.2 | h | β− | 0.296 | 76.0 |
| | | | | β− | 0.272 | 22.0 |
| | | | | γ | 0.399 | 87.9 |
| 69 | Tm-175 | 15.2 | m | β− | 0.298 | 37.1 |
| | | | | β− | 0.000 | 19.0 |
| | | | | β− | 0.531 | 12.4 |
| | | | | γ | 0.515 | 65.0 |
| | | | | γ | 0.941 | 14.6 |
| | | | | γ | 0.364 | 12.7 |
| | | | | γ | 0.982 | 10.2 |
| 70 | Yb-162 | 18.9 | m | EC | 1.487 | 73.0 |
| | | | | EC | 1.650 | 15.0 |
| | | | | γ | 0.163 | 40.0 |
| | | | | γ | 0.119 | 34.0 |
| 70 | Yb-163 | 11.1 | m | EC | 2.420 | 25.0 |
| | | | | EC | 3.417 | 14.0 |
| | | | | EC | 3.431 | 14.0 |
| | | | | EC | 1.598 | 12.0 |
| | | | | γ | 0.860 | 24.0 |
| | | | | γ | 0.064 | 15.0 |
| 70 | Yb-164 | 75.8 | m | EC | 1.100 | 96.5 |
| 70 | Yb-166 | 56.7 | h | EC | 0.223 | 100.0 |
| | | | | γ | 0.082 | 15.6 |
| 70 | Yb-167 | 17.5 | m | EC | 1.661 | 95.0 |
| | | | | γ | 0.113 | 55.0 |
| | | | | γ | 0.106 | 22.4 |
| | | | | γ | 0.176 | 20.4 |
| 70 | Yb-169 | 32.0 | d | EC | 0.531 | 81.1 |
| | | | | EC | 0.437 | 12.3 |
| | | | | γ | 0.063 | 43.6 |
| | | | | γ | 0.198 | 35.9 |
| | | | | γ | 0.177 | 22.3 |
| | | | | γ | 0.110 | 17.4 |
| | | | | γ | 0.131 | 11.4 |
| | | | | γ | 0.308 | 10.1 |
| 70 | Yb-175 | 4.2 | d | β− | 0.140 | 72.9 |
| | | | | β− | 0.019 | 20.4 |
| | | | | γ | 0.396 | 13.1 |
| 70 | Yb-177 | 1.9 | h | β− | 0.496 | 59.4 |
| | | | | β− | 0.434 | 18.5 |
| | | | | γ | 0.150 | 18.0 |
| 70 | Yb-178 | 74.0 | m | β− | 0.201 | 85.0 |
| | | | | β− | 0.071 | 15.0 |
| 71 | Lu-167 | 51.5 | m | γ | 0.030 | 16.3 |
| 71 | Lu-169 | 34.1 | h | EC | 1.332 | 25.2 |
| | | | | EC | 0.843 | 15.8 |
| | | | | EC | 2.293 | 10.0 |
| | | | | γ | 0.961 | 21.2 |
| | | | | γ | 0.191 | 18.7 |
| 71 | Lu-170 | 2.0 | d | EC | 1.094 | 14.9 |
| | | | | EC | 1.332 | 10.4 |
| 71 | Lu-171 | 8.2 | d | EC | 0.643 | 61.5 |
| | | | | EC | 1.383 | 14.0 |
| | | | | γ | 0.740 | 48.7 |
| | | | | γ | 0.019 | 14.0 |
| | | | | γ | 0.667 | 11.2 |
| 71 | Lu-172 | 6.7 | d | EC | 0.446 | 65.0 |
| | | | | γ | 1.094 | 62.0 |
| | | | | γ | 0.901 | 29.8 |
| | | | | γ | 0.182 | 20.6 |
| | | | | γ | 0.810 | 16.6 |
| | | | | γ | 0.912 | 15.2 |
| | | | | γ | 0.079 | 10.6 |
| 71 | Lu-176m1 | 3.7 | h | β− | 0.425 | 61.0 |
| | | | | β− | 0.461 | 39.0 |
| 71 | Lu-177 | 6.6 | d | β− | 0.149 | 79.4 |
| | | | | β− | 0.047 | 11.7 |
| | | | | γ | 0.208 | 10.4 |
| 71 | Lu-178 | 28.4 | m | β− | 0.795 | 63.0 |
| | | | | β− | 0.754 | 29.0 |
| 71 | Lu-178m1 | 23.1 | m | β− | 0.365 | 83.7 |
| | | | | β− | 0.238 | 13.0 |
| | | | | γ | 0.426 | 97.0 |
| | | | | γ | 0.326 | 94.1 |
| | | | | γ | 0.213 | 81.4 |
| | | | | γ | 0.089 | 64.4 |
| | | | | γ | 0.093 | 17.2 |
| | | | | γ | 0.332 | 11.4 |
| 71 | Lu-179 | 4.6 | h | β− | 0.499 | 87.0 |
| | | | | β− | 0.412 | 11.0 |
| | | | | γ | 0.214 | 12.0 |
| 72 | Hf-168 | 26.0 | m | EC | 1.258 | 49.0 |
| | | | | EC | 0.905 | 11.0 |
| 72 | Hf-170 | 16.0 | h | EC | 0.267 | 43.0 |
| | | | | EC | 1.052 | 40.0 |
| | | | | EC | 0.854 | 15.0 |
| | | | | γ | 0.165 | 26.0 |
| | | | | γ | 0.621 | 18.0 |
| | | | | γ | 0.120 | 15.0 |
| | | | | γ | 0.573 | 15.0 |
| 72 | Hf-173 | 23.6 | h | EC | 1.175 | 37.4 |
| | | | | EC | 1.337 | 32.0 |
| | | | | EC | 1.165 | 21.3 |
| | | | | γ | 0.124 | 83.0 |
| | | | | γ | 0.297 | 33.9 |
| | | | | γ | 0.140 | 12.7 |
| | | | | γ | 0.311 | 10.7 |
| 72 | Hf-177m1 | 51.4 | m | γ | 0.277 | 76.1 |
| | | | | γ | 0.295 | 70.0 |
| | | | | γ | 0.327 | 69.0 |
| | | | | γ | 0.312 | 59.0 |
| | | | | γ | 0.214 | 41.1 |
| | | | | γ | 0.638 | 20.3 |
| | | | | γ | 0.607 | 11.6 |
| 72 | Hf-179m2 | 25.1 | d | γ | 0.454 | 68.0 |
| | | | | γ | 0.363 | 39.6 |
| | | | | γ | 0.123 | 27.7 |
| | | | | γ | 0.146 | 27.1 |
| | | | | γ | 0.193 | 21.5 |
| | | | | γ | 0.410 | 21.5 |
| | | | | γ | 0.316 | 20.3 |
| | | | | γ | 0.170 | 19.4 |
| | | | | γ | 0.236 | 18.8 |
| | | | | γ | 0.269 | 11.3 |
| 72 | Hf-180m1 | 5.5 | h | γ | 0.332 | 94.0 |
| | | | | γ | 0.443 | 82.0 |
| | | | | γ | 0.215 | 81.6 |
| | | | | γ | 0.058 | 48.0 |
| | | | | γ | 0.093 | 16.5 |
| | | | | γ | 0.501 | 14.2 |
| 72 | Hf-181 | 42.4 | d | β− | 0.121 | 93.0 |
| | | | | γ | 0.482 | 80.5 |
| | | | | γ | 0.133 | 43.3 |
| | | | | γ | 0.346 | 15.1 |
| 72 | Hf-182m1 | 61.5 | m | β− | 0.129 | 43.0 |
| | | | | β− | 0.296 | 10.0 |
| | | | | γ | 0.344 | 46.0 |
| | | | | γ | 0.224 | 38.0 |
| | | | | γ | 0.507 | 23.0 |
| | | | | γ | 0.943 | 22.0 |
| | | | | γ | 0.456 | 20.0 |
| | | | | γ | 0.051 | 13.2 |
| | | | | γ | 0.800 | 10.8 |
| 72 | Hf-183 | 1.0 | h | β− | 0.394 | 68.6 |
| | | | | β− | 0.558 | 28.7 |
| | | | | β− | 0.738 | 14.0 |
| | | | | γ | 0.784 | 65.5 |
| | | | | γ | 0.073 | 38.0 |
| | | | | γ | 0.459 | 29.8 |
| 72 | Hf-184 | 4.1 | h | β− | 0.378 | 47.0 |
| | | | | β− | 0.229 | 36.0 |
| | | | | β− | 0.290 | 15.2 |
| | | | | γ | 0.139 | 46.0 |
| | | | | γ | 0.345 | 35.0 |
| | | | | γ | 0.181 | 14.1 |
| 73 | Ta-172 | 36.8 | m | EC | 3.280 | 14.0 |
| | | | | EC | 3.500 | 12.0 |
| | | | | γ | 0.214 | 55.0 |
| | | | | γ | 0.095 | 21.3 |
| | | | | γ | 1.109 | 14.8 |
| 73 | Ta-173 | 3.1 | h | EC | 2.720 | 28.0 |
| | | | | EC | 2.548 | 27.3 |
| | | | | γ | 0.172 | 17.5 |
| 73 | Ta-174 | 1.1 | h | β+ | 1.141 | 17.7 |
| | | | | EC | 3.547 | 43.0 |
| | | | | EC | 3.753 | 11.0 |
| | | | | γ | 0.207 | 60.0 |
| | | | | γ | 0.091 | 15.9 |
| 73 | Ta-175 | 10.5 | h | EC | 1.732 | 26.3 |
| | | | | EC | 1.999 | 12.0 |
| | | | | γ | 0.207 | 14.0 |
| | | | | γ | 0.349 | 12.0 |
| | | | | γ | 0.267 | 10.8 |
| 73 | Ta-176 | 8.1 | h | EC | 1.962 | 11.0 |
| | | | | γ | 1.159 | 24.7 |
| | | | | γ | 0.088 | 11.9 |
| 73 | Ta-177 | 56.4 | h | EC | 1.166 | 62.0 |
| | | | | EC | 1.053 | 35.0 |
| | | | | γ | 0.113 | 11.4 |
| 73 | Ta-178 | 2.4 | h | EC | 0.790 | 64.2 |
| | | | | EC | 0.458 | 35.8 |
| | | | | γ | 0.426 | 97.0 |
| | | | | γ | 0.326 | 94.1 |
| | | | | γ | 0.213 | 80.8 |
| | | | | γ | 0.089 | 66.9 |
| | | | | γ | 0.332 | 31.2 |
| | | | | γ | 0.093 | 17.3 |
| 73 | Ta-180 | 8.2 | h | β− | 0.221 | 10.4 |
| | | | | EC | 0.846 | 61.0 |
| | | | | EC | 0.752 | 24.9 |
| 73 | Ta-182m2 | 15.8 | m | γ | 0.172 | 48.0 |
| | | | | γ | 0.147 | 36.5 |
| | | | | γ | 0.185 | 24.0 |
| 73 | Ta-183 | 5.1 | d | β− | 0.191 | 93.0 |
| | | | | γ | 0.246 | 27.2 |
| | | | | γ | 0.354 | 11.6 |
| | | | | γ | 0.108 | 11.2 |
| 73 | Ta-184 | 8.7 | h | β− | 0.399 | 82.0 |
| | | | | β− | 0.381 | 14.7 |
| | | | | γ | 0.414 | 72.0 |
| | | | | γ | 0.253 | 44.0 |
| | | | | γ | 0.921 | 32.0 |
| | | | | γ | 0.111 | 23.7 |
| | | | | γ | 0.318 | 22.8 |
| | | | | γ | 0.903 | 15.0 |
| | | | | γ | 0.792 | 14.5 |
| | | | | γ | 0.537 | 12.7 |
| | | | | γ | 0.384 | 12.5 |
| | | | | γ | 0.215 | 11.4 |
| | | | | γ | 0.461 | 10.7 |
| | | | | γ | 0.895 | 10.7 |
| 73 | Ta-185 | 49.4 | m | β− | 0.639 | 68.0 |
| | | | | β− | 0.669 | 27.0 |
| | | | | γ | 0.178 | 25.7 |
| | | | | γ | 0.174 | 22.6 |
| 73 | Ta-186 | 10.5 | m | β− | 0.851 | 58.0 |
| | | | | β− | 1.532 | 16.0 |
| | | | | β− | 1.121 | 10.0 |
| | | | | γ | 0.198 | 50.0 |
| | | | | γ | 0.215 | 42.0 |
| | | | | γ | 0.511 | 37.0 |
| | | | | γ | 0.738 | 29.0 |
| | | | | γ | 0.615 | 28.0 |
| | | | | γ | 0.122 | 25.0 |
| | | | | γ | 0.418 | 12.5 |
| | | | | γ | 0.739 | 10.0 |
| 74 | W-174 | 33.2 | m | γ | 0.035 | 15.3 |
| | | | | γ | 0.429 | 12.7 |
| 74 | W-175 | 35.2 | m | γ | 0.270 | 12.0 |
| 74 | W-177 | 132.4 | m | EC | 1.796 | 32.6 |
| | | | | EC | 0.760 | 15.8 |
| | | | | EC | 0.501 | 11.4 |
| | | | | γ | 0.116 | 47.3 |
| | | | | γ | 0.186 | 15.2 |
| | | | | γ | 0.427 | 12.5 |
| 74 | W-178 | 21.6 | d | EC | 0.091 | 100.0 |
| 74 | W-179 | 37.1 | m | EC | 1.032 | 99.2 |
| | | | | γ | 0.031 | 18.0 |
| 74 | W-187 | 24.0 | h | β− | 0.193 | 66.2 |
| | | | | β− | 0.457 | 16.9 |
| | | | | γ | 0.686 | 33.2 |
| | | | | γ | 0.480 | 26.6 |
| | | | | γ | 0.072 | 13.6 |
| | | | | γ | 0.134 | 10.4 |
| 74 | W-190 | 30.0 | m | β− | 0.310 | 100.0 |
| | | | | γ | 0.158 | 39.0 |
| | | | | γ | 0.162 | 11.0 |
| 75 | Re-177 | 14.0 | m | γ | 0.197 | 10.0 |
| 75 | Re-178 | 13.2 | m | β+ | 1.535 | 10.0 |
| | | | | β+ | 1.644 | 10.0 |
| | | | | EC | 4.417 | 12.0 |
| | | | | γ | 0.237 | 44.5 |
| | | | | γ | 0.106 | 23.4 |
| 75 | Re-179 | 19.5 | m | EC | 1.997 | 44.0 |
| | | | | EC | 1.943 | 19.0 |
| | | | | EC | 1.037 | 17.6 |
| | | | | γ | 0.430 | 28.1 |
| | | | | γ | 0.290 | 27.0 |
| | | | | γ | 1.680 | 13.0 |
| | | | | γ | 0.415 | 10.6 |
| 75 | Re-181 | 19.9 | h | EC | 0.377 | 22.7 |
| | | | | EC | 1.017 | 16.0 |
| | | | | EC | 1.377 | 15.0 |
| | | | | EC | 1.267 | 10.3 |
| | | | | γ | 0.366 | 56.0 |
| | | | | γ | 0.361 | 20.0 |
| 75 | Re-182 | 64.2 | h | EC | 0.822 | 24.0 |
| | | | | EC | 0.840 | 23.0 |
| | | | | EC | 1.043 | 16.4 |
| | | | | EC | 0.970 | 14.0 |
| | | | | γ | 0.229 | 25.8 |
| | | | | γ | 0.068 | 22.2 |
| | | | | γ | 1.121 | 22.1 |
| | | | | γ | 1.221 | 17.5 |
| | | | | γ | 0.100 | 16.5 |
| | | | | γ | 1.231 | 14.9 |
| | | | | γ | 0.169 | 11.4 |
| | | | | γ | 1.076 | 10.6 |
| | | | | γ | 0.351 | 10.3 |
| 75 | Re-182m1 | 14.1 | h | EC | 1.511 | 37.0 |
| | | | | EC | 1.426 | 29.0 |
| | | | | EC | 2.700 | 14.0 |
| | | | | γ | 0.068 | 38.0 |
| | | | | γ | 1.121 | 32.0 |
| | | | | γ | 1.222 | 25.0 |
| | | | | γ | 1.189 | 15.1 |
| | | | | γ | 0.100 | 14.4 |
| 75 | Re-184 | 35.4 | d | EC | 0.578 | 76.5 |
| | | | | EC | 0.475 | 17.8 |
| | | | | γ | 0.903 | 38.1 |
| | | | | γ | 0.792 | 37.7 |
| | | | | γ | 0.111 | 17.2 |
| | | | | γ | 0.895 | 15.7 |
| 75 | Re-186 | 3.7 | d | β− | 0.359 | 71.0 |
| | | | | β− | 0.306 | 21.5 |
| 75 | Re-188 | 17.0 | h | β− | 0.795 | 70.7 |
| | | | | β− | 0.729 | 25.8 |
| | | | | γ | 0.155 | 15.5 |
| 75 | Re-188m1 | 18.6 | m | γ | 0.064 | 22.3 |
| | | | | γ | 0.106 | 11.5 |
| 75 | Re-189 | 24.3 | h | β− | 0.335 | 62.0 |
| | | | | β− | 0.253 | 13.0 |
| 75 | Re-190m1 | 3.2 | h | β− | 0.539 | 17.1 |
| | | | | γ | 0.187 | 27.8 |
| | | | | γ | 0.605 | 14.9 |
| | | | | γ | 0.558 | 14.3 |
| | | | | γ | 0.569 | 13.7 |
| | | | | γ | 0.361 | 12.1 |
| | | | | γ | 0.119 | 11.1 |
| | | | | γ | 0.371 | 10.3 |
| 76 | Os-180 | 21.5 | m | EC | 1.455 | 99.9 |
| | | | | γ | 0.020 | 17.0 |
| 76 | Os-181 | 105.0 | m | EC | 2.528 | 17.0 |
| | | | | EC | 2.133 | 13.0 |
| | | | | γ | 0.239 | 44.0 |
| | | | | γ | 0.827 | 19.9 |
| | | | | γ | 0.118 | 12.7 |
| 76 | Os-182 | 21.8 | h | EC | 0.330 | 62.1 |
| | | | | EC | 0.577 | 27.0 |
| | | | | γ | 0.510 | 52.4 |
| | | | | γ | 0.180 | 34.1 |
| 76 | Os-183 | 13.0 | h | EC | 1.654 | 73.8 |
| | | | | EC | 1.486 | 17.7 |
| | | | | γ | 0.382 | 91.6 |
| | | | | γ | 0.114 | 21.1 |
| 76 | Os-183m1 | 9.9 | h | EC | 1.219 | 45.6 |
| | | | | EC | 1.213 | 18.6 |
| | | | | EC | 2.321 | 11.0 |
| | | | | γ | 1.102 | 49.2 |
| | | | | γ | 1.108 | 22.5 |
| 76 | Os-191 | 15.4 | d | β− | 0.038 | 100.0 |
| | | | | γ | 0.129 | 26.5 |
| 76 | Os-193 | 29.8 | h | β− | 0.387 | 59.0 |
| | | | | β− | 0.358 | 17.0 |
| | | | | β− | 0.333 | 10.6 |
| 76 | Os-196 | 34.9 | m | β− | 0.393 | 85.0 |
| 77 | Ir-182 | 15.0 | m | β+ | 2.001 | 21.0 |
| | | | | β+ | 1.874 | 17.0 |
| | | | | EC | 5.160 | 14.0 |
| | | | | EC | 5.433 | 14.0 |
| | | | | γ | 0.274 | 40.0 |
| | | | | γ | 0.127 | 35.0 |
| 77 | Ir-183 | 58.0 | m | EC | 3.202 | 14.0 |
| | | | | EC | 3.460 | 12.0 |
| 77 | Ir-184 | 3.1 | h | EC | 2.198 | 12.0 |
| | | | | γ | 0.264 | 64.4 |
| | | | | γ | 0.120 | 30.8 |
| | | | | γ | 0.390 | 26.1 |
| | | | | γ | 0.961 | 10.2 |
| 77 | Ir-185 | 14.4 | h | γ | 0.254 | 13.3 |
| | | | | γ | 1.829 | 10.1 |
| 77 | Ir-186 | 16.6 | h | EC | 1.940 | 20.6 |
| | | | | EC | 1.750 | 10.5 |
| | | | | γ | 0.297 | 62.3 |
| | | | | γ | 0.137 | 41.4 |
| | | | | γ | 0.435 | 33.9 |
| 77 | Ir-186m1 | 1.9 | h | EC | 2.077 | 18.6 |
| | | | | EC | 3.064 | 14.0 |
| | | | | γ | 0.137 | 23.0 |
| | | | | γ | 0.767 | 18.4 |
| | | | | γ | 0.630 | 15.6 |
| | | | | γ | 0.773 | 11.7 |
| 77 | Ir-187 | 10.5 | h | EC | 1.492 | 24.0 |
| | | | | EC | 1.427 | 20.0 |
| | | | | EC | 0.515 | 13.0 |
| | | | | EC | 1.001 | 11.4 |
| | | | | EC | 1.428 | 10.0 |
| 77 | Ir-188 | 41.5 | h | EC | 0.577 | 26.6 |
| | | | | γ | 0.155 | 29.6 |
| | | | | γ | 2.215 | 18.6 |
| | | | | γ | 0.633 | 17.9 |
| | | | | γ | 0.478 | 14.7 |
| 77 | Ir-189 | 13.2 | d | EC | 0.537 | 40.0 |
| | | | | EC | 0.467 | 35.0 |
| | | | | EC | 0.442 | 11.6 |
| | | | | EC | 0.261 | 10.1 |
| 77 | Ir-190 | 11.8 | d | EC | 0.272 | 49.0 |
| | | | | EC | 0.791 | 26.0 |
| | | | | EC | 0.370 | 10.2 |
| | | | | γ | 0.187 | 52.0 |
| | | | | γ | 0.605 | 39.9 |
| | | | | γ | 0.519 | 34.0 |
| | | | | γ | 0.558 | 30.1 |
| | | | | γ | 0.569 | 28.5 |
| | | | | γ | 0.407 | 23.9 |
| | | | | γ | 0.371 | 22.8 |
| | | | | γ | 0.361 | 13.0 |
| 77 | Ir-190m2 | 3.1 | h | EC | 0.625 | 91.4 |
| | | | | γ | 0.617 | 90.1 |
| | | | | γ | 0.503 | 89.4 |
| | | | | γ | 0.361 | 86.7 |
| | | | | γ | 0.187 | 64.2 |
| 77 | Ir-194 | 19.3 | h | β− | 0.840 | 85.4 |
| | | | | γ | 0.328 | 13.1 |
| 77 | Ir-195 | 2.3 | h | β− | 0.320 | 57.0 |
| | | | | β− | 0.332 | 25.0 |
| | | | | β− | 0.371 | 13.0 |
| | | | | γ | 0.099 | 10.0 |
| 77 | Ir-195m1 | 3.7 | h | β− | 0.112 | 36.0 |
| | | | | β− | 0.309 | 33.0 |
| | | | | γ | 0.099 | 10.7 |
| 77 | Ir-196m1 | 1.4 | h | β− | 0.390 | 80.0 |
| | | | | γ | 0.394 | 97.0 |
| | | | | γ | 0.521 | 96.0 |
| | | | | γ | 0.356 | 94.0 |
| | | | | γ | 0.447 | 94.0 |
| | | | | γ | 0.647 | 91.0 |
| | | | | γ | 0.103 | 18.4 |
| 78 | Pt-184 | 17.3 | m | EC | 1.376 | 29.0 |
| | | | | EC | 1.193 | 23.3 |
| | | | | EC | 1.215 | 11.5 |
| | | | | γ | 0.155 | 31.0 |
| | | | | γ | 0.192 | 27.0 |
| | | | | γ | 0.071 | 23.0 |
| | | | | γ | 0.548 | 23.0 |
| | | | | γ | 0.731 | 12.8 |
| 78 | Pt-186 | 2.1 | h | EC | 0.691 | 86.0 |
| | | | | γ | 0.689 | 70.0 |
| 78 | Pt-187 | 2.4 | h | EC | 2.890 | 14.1 |
| | | | | EC | 2.688 | 13.4 |
| | | | | EC | 2.181 | 12.9 |
| | | | | EC | 2.894 | 10.4 |
| 78 | Pt-188 | 10.2 | d | EC | 0.329 | 44.0 |
| | | | | EC | 0.336 | 27.8 |
| | | | | EC | 0.524 | 15.0 |
| | | | | EC | 0.046 | 12.8 |
| | | | | γ | 0.188 | 19.1 |
| | | | | γ | 0.195 | 18.4 |
| 78 | Pt-189 | 10.9 | h | EC | 1.259 | 21.0 |
| | | | | EC | 1.980 | 21.0 |
| | | | | EC | 1.662 | 15.0 |
| | | | | EC | 1.803 | 13.0 |
| | | | | EC | 1.886 | 12.0 |
| 78 | Pt-191 | 2.8 | d | EC | 0.469 | 33.0 |
| | | | | EC | 1.008 | 30.0 |
| | | | | EC | 0.926 | 25.0 |
| | | | | EC | 0.657 | 12.7 |
| | | | | EC | 0.829 | 11.4 |
| | | | | γ | 0.539 | 15.9 |
| 78 | Pt-195 | 4.0 | d | γ | 0.099 | 11.7 |
| 78 | Pt-197 | 19.9 | h | β− | 0.198 | 81.2 |
| | | | | β− | 0.225 | 10.6 |
| | | | | γ | 0.077 | 17.0 |
| 78 | Pt-197m1 | 95.4 | m | γ | 0.347 | 11.1 |
| 78 | Pt-199 | 30.8 | m | β− | 0.613 | 70.7 |
| | | | | β− | 0.393 | 12.0 |
| | | | | γ | 0.543 | 11.7 |
| 78 | Pt-200 | 12.6 | h | γ | 0.076 | 13.4 |
| 78 | Pt-202 | 44.0 | h | β− | 0.655 | 100.0 |
| 79 | Au-186 | 10.7 | m | β+ | 2.227 | 16.0 |
| | | | | γ | 0.192 | 62.0 |
| | | | | γ | 0.299 | 25.4 |
| | | | | γ | 0.765 | 10.5 |
| 79 | Au-190 | 42.8 | m | EC | 2.059 | 12.0 |
| | | | | EC | 3.844 | 11.0 |
| | | | | γ | 0.296 | 90.0 |
| | | | | γ | 0.302 | 29.7 |
| | | | | γ | 0.598 | 12.0 |
| 79 | Au-191 | 3.2 | h | EC | 0.816 | 30.0 |
| | | | | EC | 1.880 | 12.0 |
| | | | | EC | 1.890 | 12.0 |
| | | | | EC | 1.612 | 11.0 |
| | | | | γ | 0.586 | 15.0 |
| 79 | Au-192 | 4.9 | h | EC | 3.514 | 14.0 |
| | | | | EC | 1.181 | 11.4 |
| | | | | γ | 0.317 | 59.0 |
| | | | | γ | 0.296 | 23.0 |
| 79 | Au-193 | 17.7 | h | EC | 0.887 | 26.0 |
| | | | | EC | 1.061 | 20.0 |
| | | | | EC | 0.805 | 15.7 |
| | | | | EC | 1.075 | 15.0 |
| | | | | EC | 0.961 | 12.4 |
| 79 | Au-194 | 38.0 | h | EC | 2.173 | 29.0 |
| | | | | EC | 2.509 | 24.0 |
| | | | | EC | 0.704 | 11.1 |
| | | | | γ | 0.328 | 60.4 |
| | | | | γ | 0.294 | 10.6 |
| 79 | Au-196 | 6.2 | d | EC | 1.151 | 67.0 |
| | | | | EC | 0.818 | 24.6 |
| | | | | γ | 0.356 | 87.0 |
| | | | | γ | 0.333 | 22.9 |
| 79 | Au-196m2 | 9.6 | h | γ | 0.148 | 43.5 |
| | | | | γ | 0.188 | 30.0 |
| 79 | Au-198 | 2.7 | d | β− | 0.315 | 99.0 |
| | | | | γ | 0.412 | 95.6 |
| 79 | Au-198m1 | 2.3 | d | γ | 0.215 | 77.0 |
| | | | | γ | 0.097 | 69.0 |
| | | | | γ | 0.180 | 49.0 |
| | | | | γ | 0.204 | 38.0 |
| | | | | γ | 0.334 | 18.0 |
| 79 | Au-199 | 3.1 | d | β− | 0.082 | 72.0 |
| | | | | β− | 0.067 | 21.5 |
| | | | | γ | 0.158 | 40.0 |
| 79 | Au-200 | 48.4 | m | β− | 0.838 | 79.0 |
| | | | | β− | 0.199 | 10.9 |
| | | | | γ | 0.368 | 19.0 |
| | | | | γ | 1.225 | 10.7 |
| 79 | Au-200m1 | 18.7 | h | β− | 0.169 | 84.0 |
| | | | | γ | 0.368 | 86.1 |
| | | | | γ | 0.498 | 82.0 |
| | | | | γ | 0.579 | 80.0 |
| | | | | γ | 0.256 | 79.0 |
| | | | | γ | 0.760 | 74.0 |
| | | | | γ | 0.181 | 62.0 |
| | | | | γ | 0.333 | 14.0 |
| 80 | Hg-190 | 20.0 | m | EC | 1.348 | 85.0 |
| | | | | EC | 1.520 | 10.0 |
| | | | | γ | 0.143 | 68.0 |
| 80 | Hg-191 | 50.8 | m | EC | 2.674 | 10.2 |
| | | | | γ | 0.253 | 57.0 |
| | | | | γ | 0.420 | 19.0 |
| | | | | γ | 0.579 | 18.0 |
| | | | | γ | 0.274 | 13.0 |
| | | | | γ | 0.241 | 12.0 |
| 80 | Hg-192 | 4.9 | h | EC | 0.458 | 66.0 |
| | | | | EC | 0.607 | 19.7 |
| | | | | γ | 0.275 | 52.0 |
| 80 | Hg-193 | 3.8 | h | EC | 2.118 | 34.0 |
| | | | | EC | 1.224 | 24.0 |
| | | | | EC | 2.305 | 15.0 |
| | | | | EC | 1.961 | 13.0 |
| | | | | γ | 0.187 | 15.1 |
| | | | | γ | 0.382 | 15.0 |
| | | | | γ | 0.861 | 12.1 |
| 80 | Hg-195 | 10.5 | h | EC | 1.509 | 71.0 |
| | | | | EC | 1.570 | 10.0 |
| 80 | Hg-195m1 | 41.6 | h | EC | 1.427 | 32.0 |
| | | | | γ | 0.262 | 31.0 |
| 80 | Hg-197 | 64.1 | h | EC | 0.523 | 96.7 |
| | | | | γ | 0.077 | 18.7 |
| 80 | Hg-197m1 | 23.8 | h | γ | 0.134 | 33.5 |
| 80 | Hg-199 | 42.7 | m | γ | 0.158 | 52.3 |
| | | | | γ | 0.374 | 13.8 |
| 80 | Hg-203 | 46.6 | d | β− | 0.058 | 100.0 |
| | | | | γ | 0.279 | 81.6 |
| 81 | Tl-194 | 33.0 | m | β+ | 1.769 | 21.0 |
| | | | | EC | 4.942 | 25.0 |
| | | | | EC | 5.370 | 12.0 |
| | | | | γ | 0.428 | 75.0 |
| | | | | γ | 0.645 | 10.8 |
| 81 | Tl-194m1 | 32.8 | m | EC | 3.710 | 26.0 |
| | | | | EC | 3.156 | 15.8 |
| | | | | EC | 3.807 | 11.0 |
| | | | | EC | 3.355 | 10.0 |
| | | | | γ | 0.428 | 99.0 |
| | | | | γ | 0.636 | 99.0 |
| | | | | γ | 0.749 | 76.0 |
| | | | | γ | 0.735 | 22.0 |
| 81 | Tl-195 | 1.2 | h | EC | 1.444 | 15.4 |
| | | | | EC | 1.923 | 13.5 |
| | | | | EC | 2.244 | 12.0 |
| | | | | γ | 0.564 | 11.2 |
| | | | | γ | 0.884 | 10.6 |
| 81 | Tl-196 | 1.8 | h | EC | 3.904 | 28.0 |
| | | | | γ | 0.426 | 83.0 |
| | | | | γ | 0.611 | 11.8 |
| 81 | Tl-196m1 | 1.4 | h | EC | 2.884 | 66.0 |
| | | | | EC | 2.379 | 15.0 |
| | | | | γ | 0.635 | 94.0 |
| | | | | γ | 0.426 | 92.0 |
| | | | | γ | 0.695 | 90.0 |
| | | | | γ | 0.505 | 13.0 |
| 81 | Tl-197 | 2.8 | h | EC | 2.200 | 52.0 |
| | | | | EC | 1.622 | 21.0 |
| | | | | γ | 0.426 | 13.0 |
| 81 | Tl-198 | 5.3 | h | EC | 1.627 | 11.7 |
| | | | | γ | 0.412 | 79.0 |
| | | | | γ | 0.676 | 10.6 |
| 81 | Tl-198m1 | 1.9 | h | EC | 2.320 | 24.0 |
| | | | | γ | 0.412 | 59.0 |
| | | | | γ | 0.637 | 59.0 |
| | | | | γ | 0.587 | 54.4 |
| | | | | γ | 0.283 | 27.0 |
| 81 | Tl-199 | 7.4 | h | EC | 1.488 | 48.0 |
| | | | | EC | 1.033 | 29.0 |
| | | | | γ | 0.455 | 12.4 |
| | | | | γ | 0.208 | 12.3 |
| 81 | Tl-200 | 26.1 | h | EC | 0.882 | 30.0 |
| | | | | EC | 2.088 | 24.9 |
| | | | | EC | 0.680 | 13.6 |
| | | | | γ | 0.368 | 87.0 |
| | | | | γ | 1.206 | 30.0 |
| | | | | γ | 0.579 | 13.7 |
| | | | | γ | 0.828 | 10.8 |
| 81 | Tl-201 | 3.0 | d | EC | 0.314 | 41.4 |
| | | | | EC | 0.479 | 26.3 |
| | | | | EC | 0.481 | 20.9 |
| | | | | EC | 0.449 | 10.9 |
| | | | | γ | 0.167 | 10.0 |
| 81 | Tl-202 | 12.3 | d | EC | 0.923 | 94.3 |
| | | | | γ | 0.440 | 91.5 |
| 82 | Pb-194 | 10.7 | m | EC | 1.834 | 23.8 |
| | | | | EC | 1.101 | 23.7 |
| | | | | γ | 0.582 | 18.9 |
| | | | | γ | 1.519 | 16.5 |
| | | | | γ | 0.204 | 16.3 |
| 82 | Pb-195 | 15.0 | m | EC | 3.283 | 18.6 |
| | | | | EC | 3.767 | 14.0 |
| | | | | γ | 0.384 | 91.0 |
| | | | | γ | 0.394 | 44.2 |
| | | | | γ | 0.878 | 24.3 |
| | | | | γ | 0.708 | 14.1 |
| 82 | Pb-196 | 37.0 | m | EC | 1.381 | 33.0 |
| | | | | EC | 1.770 | 25.0 |
| | | | | EC | 1.883 | 13.0 |
| | | | | EC | 1.642 | 11.5 |
| | | | | EC | 1.944 | 11.0 |
| | | | | γ | 0.253 | 27.0 |
| | | | | γ | 0.502 | 27.0 |
| | | | | γ | 0.192 | 11.1 |
| | | | | γ | 0.367 | 11.1 |
| 82 | Pb-197m1 | 42.9 | m | EC | 3.303 | 22.0 |
| | | | | γ | 0.386 | 74.0 |
| | | | | γ | 0.223 | 25.0 |
| | | | | γ | 0.388 | 25.0 |
| | | | | γ | 0.774 | 14.2 |
| 82 | Pb-198 | 2.4 | h | EC | 1.160 | 27.0 |
| | | | | EC | 0.794 | 24.0 |
| | | | | EC | 1.277 | 22.8 |
| | | | | EC | 0.585 | 10.5 |
| | | | | γ | 0.290 | 36.0 |
| | | | | γ | 0.365 | 19.0 |
| | | | | γ | 0.173 | 18.2 |
| 82 | Pb-199 | 90.0 | m | EC | 2.830 | 28.0 |
| | | | | EC | 2.463 | 16.0 |
| | | | | EC | 1.328 | 12.0 |
| | | | | γ | 0.367 | 44.0 |
| 82 | Pb-199m1 | 12.2 | m | γ | 0.424 | 19.6 |
| 82 | Pb-200 | 21.5 | h | EC | 0.657 | 68.0 |
| | | | | EC | 0.279 | 14.3 |
| | | | | γ | 0.148 | 38.2 |
| 82 | Pb-201 | 9.3 | h | EC | 1.593 | 52.0 |
| | | | | EC | 0.647 | 12.3 |
| | | | | γ | 0.331 | 77.0 |
| 82 | Pb-202m1 | 3.5 | h | γ | 0.961 | 89.9 |
| | | | | γ | 0.422 | 84.0 |
| | | | | γ | 0.787 | 49.0 |
| | | | | γ | 0.657 | 31.7 |
| 82 | Pb-203 | 51.9 | h | EC | 0.696 | 95.3 |
| | | | | γ | 0.279 | 80.9 |
| 82 | Pb-204 | 66.9 | m | γ | 0.899 | 99.2 |
| | | | | γ | 0.375 | 94.2 |
| | | | | γ | 0.912 | 91.5 |
| 82 | Pb-209 | 3.2 | h | β− | 0.198 | 100.0 |
| 82 | Pb-211 | 36.1 | m | β− | 0.471 | 91.3 |
| 82 | Pb-212 | 10.6 | h | β− | 0.094 | 83.1 |
| | | | | β− | 0.172 | 11.9 |
| | | | | γ | 0.239 | 43.6 |
| 82 | Pb-214 | 27.1 | m | β− | 0.206 | 45.9 |
| | | | | β− | 0.226 | 40.2 |
| | | | | β− | 0.335 | 11.0 |
| | | | | γ | 0.352 | 35.6 |
| | | | | γ | 0.295 | 18.4 |
| 83 | Bi-200 | 36.4 | m | EC | 3.964 | 29.0 |
| | | | | EC | 3.690 | 25.0 |
| | | | | EC | 3.719 | 22.0 |
| | | | | γ | 1.026 | 100.0 |
| | | | | γ | 0.462 | 98.0 |
| | | | | γ | 0.420 | 91.0 |
| | | | | γ | 0.245 | 46.0 |
| 83 | Bi-200m1 | 31.0 | m | γ | 1.027 | 96.0 |
| | | | | γ | 0.462 | 40.0 |
| | | | | γ | 0.420 | 22.8 |
| 83 | Bi-201 | 103.0 | m | EC | 3.211 | 19.0 |
| | | | | γ | 0.629 | 26.0 |
| | | | | γ | 0.936 | 12.2 |
| | | | | γ | 1.014 | 11.6 |
| | | | | γ | 0.786 | 10.3 |
| 83 | Bi-202 | 1.7 | h | EC | 3.161 | 11.0 |
| | | | | γ | 0.961 | 99.3 |
| | | | | γ | 0.422 | 84.0 |
| | | | | γ | 0.657 | 60.6 |
| 83 | Bi-203 | 11.8 | h | EC | 0.534 | 21.5 |
| | | | | EC | 0.579 | 20.3 |
| | | | | γ | 0.820 | 30.0 |
| | | | | γ | 0.825 | 14.8 |
| | | | | γ | 0.897 | 13.2 |
| | | | | γ | 1.847 | 11.6 |
| 83 | Bi-204 | 11.2 | h | EC | 1.270 | 14.7 |
| | | | | EC | 1.511 | 11.3 |
| | | | | EC | 1.411 | 10.0 |
| | | | | EC | 2.182 | 10.0 |
| | | | | γ | 0.899 | 99.0 |
| | | | | γ | 0.375 | 82.0 |
| | | | | γ | 0.984 | 59.0 |
| | | | | γ | 0.912 | 13.6 |
| | | | | γ | 0.671 | 11.4 |
| | | | | γ | 0.912 | 11.2 |
| | | | | γ | 0.918 | 10.9 |
| 83 | Bi-205 | 15.3 | d | EC | 0.944 | 32.6 |
| | | | | EC | 2.002 | 16.2 |
| | | | | EC | 1.094 | 11.2 |
| | | | | γ | 1.764 | 32.5 |
| | | | | γ | 0.703 | 31.1 |
| | | | | γ | 0.988 | 16.1 |
| 83 | Bi-206 | 6.2 | d | EC | 0.355 | 49.1 |
| | | | | EC | 0.479 | 43.7 |
| | | | | γ | 0.803 | 99.0 |
| | | | | γ | 0.881 | 66.2 |
| | | | | γ | 0.516 | 40.8 |
| | | | | γ | 1.719 | 31.9 |
| | | | | γ | 0.537 | 30.5 |
| | | | | γ | 0.344 | 23.5 |
| | | | | γ | 0.184 | 15.8 |
| | | | | γ | 0.895 | 15.7 |
| | | | | γ | 0.497 | 15.3 |
| | | | | γ | 1.098 | 13.5 |
| | | | | γ | 0.398 | 10.8 |
| 83 | Bi-210 | 5.0 | d | β− | 0.389 | 100.0 |
| 83 | Bi-212 | 60.6 | m | α | 6.051 | 25.1 |
| | | | | β− | 0.834 | 55.4 |
| 83 | Bi-212m1 | 25.0 | m | α | 6.340 | 35.0 |
| | | | | α | 6.300 | 26.0 |
| 83 | Bi-213 | 45.6 | m | β− | 0.492 | 65.9 |
| | | | | β− | 0.320 | 30.8 |
| | | | | γ | 0.440 | 25.9 |
| 83 | Bi-214 | 19.9 | m | β− | 1.269 | 19.1 |
| | | | | β− | 0.539 | 17.6 |
| | | | | β− | 0.525 | 17.0 |
| | | | | γ | 0.609 | 45.5 |
| | | | | γ | 1.764 | 15.3 |
| | | | | γ | 1.120 | 14.9 |
| 84 | Po-200 | 11.5 | m | α | 5.862 | 11.1 |
| 84 | Po-201 | 15.6 | m | EC | 4.045 | 24.0 |
| | | | | γ | 0.890 | 20.5 |
| | | | | γ | 0.240 | 14.6 |
| | | | | γ | 0.904 | 11.2 |
| 84 | Po-202 | 44.6 | m | γ | 0.689 | 49.0 |
| | | | | γ | 0.316 | 13.7 |
| 84 | Po-203 | 36.7 | m | EC | 3.321 | 25.0 |
| | | | | EC | 3.139 | 13.1 |
| | | | | γ | 0.909 | 55.0 |
| | | | | γ | 1.091 | 19.2 |
| | | | | γ | 0.894 | 18.7 |
| | | | | γ | 0.215 | 14.3 |
| | | | | γ | 0.513 | 11.0 |
| 84 | Po-204 | 3.5 | h | EC | 0.961 | 51.3 |
| | | | | EC | 0.696 | 23.8 |
| | | | | EC | 1.075 | 11.2 |
| | | | | γ | 0.884 | 34.3 |
| | | | | γ | 0.270 | 31.9 |
| | | | | γ | 1.016 | 27.6 |
| | | | | γ | 0.535 | 15.2 |
| | | | | γ | 0.763 | 13.2 |
| | | | | γ | 0.063 | 12.3 |
| | | | | γ | 0.137 | 11.1 |
| | | | | γ | 1.040 | 11.0 |
| 84 | Po-205 | 1.7 | h | EC | 2.552 | 39.0 |
| | | | | EC | 1.844 | 16.1 |
| | | | | EC | 2.703 | 14.0 |
| | | | | γ | 0.872 | 37.0 |
| | | | | γ | 1.001 | 28.8 |
| | | | | γ | 0.850 | 25.5 |
| | | | | γ | 0.837 | 19.2 |
| 84 | Po-206 | 8.8 | d | EC | 0.457 | 74.1 |
| | | | | EC | 0.914 | 19.3 |
| | | | | γ | 1.032 | 31.7 |
| | | | | γ | 0.511 | 23.2 |
| | | | | γ | 0.286 | 22.9 |
| | | | | γ | 0.807 | 21.8 |
| | | | | γ | 0.338 | 18.5 |
| | | | | γ | 0.522 | 15.1 |
| 84 | Po-207 | 5.8 | h | EC | 1.915 | 63.7 |
| | | | | EC | 0.849 | 21.2 |
| | | | | EC | 2.162 | 10.9 |
| | | | | γ | 0.992 | 59.2 |
| | | | | γ | 0.743 | 28.4 |
| | | | | γ | 0.912 | 17.0 |
| 85 | At-205 | 26.9 | m | γ | 0.719 | 44.0 |
| | | | | γ | 0.669 | 12.4 |
| 85 | At-206 | 30.6 | m | EC | 4.189 | 29.0 |
| | | | | EC | 4.584 | 12.0 |
| | | | | γ | 0.701 | 98.0 |
| | | | | γ | 0.477 | 86.0 |
| | | | | γ | 0.396 | 48.0 |
| | | | | γ | 0.734 | 10.2 |
| 85 | At-207 | 1.8 | h | EC | 1.600 | 17.5 |
| | | | | EC | 1.673 | 11.3 |
| | | | | γ | 0.814 | 45.0 |
| | | | | γ | 0.588 | 19.5 |
| | | | | γ | 0.301 | 12.9 |
| 85 | At-208 | 1.6 | h | EC | 1.413 | 19.5 |
| | | | | EC | 2.422 | 16.2 |
| | | | | EC | 3.454 | 11.0 |
| | | | | γ | 0.687 | 97.6 |
| | | | | γ | 0.660 | 89.0 |
| | | | | γ | 0.178 | 48.6 |
| | | | | γ | 0.845 | 19.7 |
| | | | | γ | 1.028 | 16.8 |
| | | | | γ | 0.990 | 10.7 |
| 85 | At-209 | 5.4 | h | EC | 1.171 | 70.9 |
| | | | | γ | 0.545 | 90.9 |
| | | | | γ | 0.782 | 83.3 |
| | | | | γ | 0.790 | 63.5 |
| | | | | γ | 0.195 | 23.5 |
| | | | | γ | 0.239 | 12.5 |
| 85 | At-210 | 8.1 | h | EC | 1.071 | 70.0 |
| | | | | EC | 0.955 | 19.0 |
| | | | | γ | 1.181 | 99.0 |
| | | | | γ | 0.245 | 79.0 |
| | | | | γ | 1.483 | 46.5 |
| | | | | γ | 1.437 | 29.0 |
| | | | | γ | 1.600 | 13.4 |
| 85 | At-211 | 7.2 | h | α | 5.870 | 41.8 |
| | | | | EC | 0.785 | 57.9 |
| 86 | Rn-208 | 24.4 | m | α | 6.140 | 62.0 |
| 86 | Rn-209 | 28.8 | m | α | 6.039 | 16.9 |
| | | | | EC | 3.208 | 40.0 |
| | | | | EC | 3.546 | 20.0 |
| | | | | γ | 0.408 | 50.4 |
| | | | | γ | 0.746 | 22.8 |
| | | | | γ | 0.337 | 14.5 |
| 86 | Rn-210 | 2.4 | h | α | 6.041 | 96.0 |
| 86 | Rn-211 | 14.6 | h | α | 5.784 | 17.3 |
| | | | | EC | 0.413 | 68.0 |
| | | | | γ | 0.674 | 45.4 |
| | | | | γ | 1.363 | 32.7 |
| | | | | γ | 0.678 | 29.0 |
| | | | | γ | 0.442 | 23.1 |
| | | | | γ | 1.127 | 22.2 |
| | | | | γ | 0.947 | 16.3 |
| 86 | Rn-212 | 23.9 | m | α | 6.264 | 100.0 |
| 86 | Rn-221 | 25.0 | m | α | 6.037 | 16.2 |
| | | | | β− | 0.271 | 27.2 |
| | | | | γ | 0.186 | 21.6 |
| 86 | Rn-222 | 3.8 | d | α | 5.489 | 99.9 |
| 86 | Rn-223 | 24.3 | m | β− | 0.676 | 24.0 |
| | | | | β− | 0.648 | 21.0 |
| | | | | β− | 0.438 | 14.0 |
| | | | | β− | 0.387 | 11.0 |
| 87 | Fr-212 | 20.0 | m | α | 6.262 | 16.3 |
| | | | | α | 6.383 | 10.3 |
| | | | | EC | 2.434 | 20.5 |
| | | | | EC | 3.481 | 15.4 |
| | | | | EC | 3.620 | 13.1 |
| | | | | γ | 1.275 | 46.0 |
| | | | | γ | 0.228 | 42.6 |
| | | | | γ | 1.186 | 14.1 |
| 87 | Fr-222 | 14.2 | m | β− | 0.604 | 54.0 |
| | | | | β− | 0.688 | 38.0 |
| | | | | γ | 0.206 | 50.0 |
| | | | | γ | 0.111 | 13.1 |
| 87 | Fr-223 | 22.0 | m | β− | 0.362 | 70.0 |
| | | | | β− | 0.350 | 15.0 |
| | | | | β− | 0.293 | 10.1 |
| | | | | γ | 0.050 | 34.0 |
| 88 | Ra-223 | 11.4 | d | α | 5.716 | 51.6 |
| | | | | α | 5.607 | 25.2 |
| | | | | γ | 0.269 | 13.9 |
| 88 | Ra-224 | 3.6 | d | α | 5.685 | 94.9 |
| 88 | Ra-225 | 14.9 | d | β− | 0.093 | 69.5 |
| | | | | β− | 0.105 | 30.5 |
| | | | | γ | 0.040 | 30.0 |
| 88 | Ra-227 | 42.2 | m | β− | 0.438 | 37.2 |
| | | | | β− | 0.431 | 25.2 |
| | | | | β− | 0.323 | 19.0 |
| | | | | γ | 0.027 | 14.1 |
| 88 | Ra-230 | 93.0 | m | β− | 0.218 | 40.0 |
| | | | | β− | 0.193 | 16.0 |
| | | | | β− | 0.183 | 11.0 |
| | | | | γ | 0.072 | 10.0 |
| 89 | Ac-224 | 2.8 | h | EC | 1.192 | 90.0 |
| | | | | γ | 0.216 | 52.3 |
| | | | | γ | 0.131 | 26.9 |
| 89 | Ac-225 | 9.9 | d | α | 5.830 | 50.7 |
| | | | | α | 5.793 | 18.1 |
| 89 | Ac-226 | 29.4 | h | β− | 0.281 | 49.0 |
| | | | | β− | 0.367 | 24.0 |
| | | | | β− | 0.340 | 10.0 |
| | | | | EC | 0.386 | 11.3 |
| | | | | γ | 0.230 | 26.9 |
| | | | | γ | 0.158 | 17.5 |
| 89 | Ac-228 | 6.2 | h | β− | 0.385 | 29.9 |
| | | | | β− | 0.610 | 11.7 |
| | | | | γ | 0.911 | 25.8 |
| | | | | γ | 0.969 | 15.8 |
| | | | | γ | 0.338 | 11.3 |
| 89 | Ac-229 | 62.7 | m | β− | 0.387 | 79.0 |
| 90 | Th-226 | 30.6 | m | α | 6.337 | 75.5 |
| | | | | α | 6.234 | 22.8 |
| 90 | Th-227 | 18.7 | d | α | 6.038 | 24.2 |
| | | | | α | 5.978 | 23.5 |
| | | | | α | 5.757 | 20.4 |
| | | | | γ | 0.236 | 12.9 |
| 90 | Th-231 | 25.5 | h | β− | 0.080 | 40.0 |
| | | | | β− | 0.085 | 32.0 |
| | | | | β− | 0.056 | 12.1 |
| | | | | β− | 0.080 | 12.0 |
| | | | | γ | 0.026 | 14.1 |
| 90 | Th-233 | 21.8 | m | β− | 0.411 | 50.0 |
| | | | | β− | 0.414 | 30.0 |
| | | | | β− | 0.378 | 16.0 |
| 90 | Th-234 | 24.1 | d | β− | 0.054 | 78.0 |
| | | | | β− | 0.028 | 14.0 |
| 90 | Th-236 | 37.3 | m | β− | 0.359 | 80.0 |
| 91 | Pa-227 | 38.3 | m | α | 6.466 | 42.7 |
| | | | | α | 6.416 | 12.8 |
| 91 | Pa-228 | 22.0 | h | EC | 0.720 | 31.8 |
| 91 | Pa-229 | 1.5 | d | EC | 0.311 | 93.0 |
| 91 | Pa-230 | 17.4 | d | EC | 0.359 | 39.6 |
| | | | | EC | 1.257 | 21.0 |
| | | | | γ | 0.952 | 30.0 |
| 91 | Pa-232 | 1.3 | d | β− | 0.089 | 73.0 |
| | | | | β− | 0.079 | 24.5 |
| | | | | γ | 0.969 | 42.3 |
| | | | | γ | 0.894 | 19.6 |
| | | | | γ | 0.150 | 10.4 |
| 91 | Pa-233 | 27.0 | d | β− | 0.041 | 26.7 |
| | | | | β− | 0.062 | 26.0 |
| | | | | β− | 0.071 | 24.0 |
| | | | | β− | 0.046 | 16.2 |
| | | | | γ | 0.312 | 38.5 |
| 91 | Pa-234 | 6.7 | h | β− | 0.137 | 34.0 |
| | | | | β− | 0.194 | 20.4 |
| | | | | β− | 0.137 | 12.9 |
| | | | | γ | 0.131 | 18.9 |
| | | | | γ | 0.946 | 14.0 |
| | | | | γ | 0.883 | 10.0 |
| 91 | Pa-235 | 24.4 | m | β− | 0.476 | 100.0 |
| 92 | U-229 | 58.0 | m | α | 6.360 | 12.8 |
| 92 | U-230 | 20.2 | d | α | 5.888 | 67.4 |
| | | | | α | 5.818 | 32.0 |
| 92 | U-231 | 4.2 | d | EC | 0.297 | 50.0 |
| | | | | EC | 0.279 | 50.0 |
| | | | | γ | 0.026 | 14.6 |
| 92 | U-237 | 6.8 | d | β− | 0.065 | 51.0 |
| | | | | β− | 0.069 | 42.0 |
| | | | | γ | 0.060 | 34.5 |
| | | | | γ | 0.208 | 21.2 |
| 92 | U-239 | 23.5 | m | β− | 0.390 | 69.0 |
| | | | | β− | 0.419 | 18.7 |
| | | | | γ | 0.075 | 53.2 |
| 92 | U-240 | 14.1 | h | β− | 0.108 | 75.0 |
| | | | | β− | 0.094 | 25.0 |
| 92 | U-242 | 16.8 | m | β− | 0.395 | 79.0 |
| | | | | β− | 0.370 | 13.0 |
| | | | | γ | 0.068 | 10.0 |
| 93 | Np-231 | 48.8 | m | EC | 1.700 | 88.2 |
| 93 | Np-232 | 14.7 | m | EC | 1.556 | 94.0 |
| | | | | γ | 0.327 | 53.0 |
| | | | | γ | 0.820 | 33.9 |
| | | | | γ | 0.867 | 24.9 |
| | | | | γ | 0.864 | 20.7 |
| | | | | γ | 0.282 | 20.1 |
| 93 | Np-233 | 36.2 | m | EC | 1.030 | 97.0 |
| 93 | Np-234 | 4.4 | d | EC | 0.208 | 29.0 |
| | | | | EC | 1.810 | 26.0 |
| | | | | EC | 0.239 | 17.0 |
| | | | | EC | 0.573 | 10.2 |
| | | | | γ | 1.558 | 18.5 |
| | | | | γ | 1.527 | 11.1 |
| 93 | Np-236m1 | 22.5 | h | β− | 0.178 | 20.0 |
| | | | | EC | 0.930 | 18.0 |
| 93 | Np-238 | 2.1 | d | β− | 0.072 | 44.8 |
| | | | | β− | 0.412 | 41.1 |
| | | | | β− | 0.060 | 11.5 |
| | | | | γ | 0.984 | 25.1 |
| | | | | γ | 1.029 | 18.2 |
| 93 | Np-239 | 2.4 | d | β− | 0.126 | 45.0 |
| | | | | β− | 0.093 | 44.0 |
| | | | | γ | 0.106 | 25.3 |
| | | | | γ | 0.278 | 14.5 |
| | | | | γ | 0.228 | 10.7 |
| 93 | Np-240 | 61.9 | m | β− | 0.276 | 79.0 |
| | | | | β− | 0.347 | 17.0 |
| | | | | β− | 0.376 | 10.0 |
| | | | | γ | 0.566 | 27.6 |
| | | | | γ | 0.974 | 25.9 |
| | | | | γ | 0.601 | 20.0 |
| | | | | γ | 0.896 | 14.8 |
| | | | | γ | 0.448 | 13.4 |
| 93 | Np-241 | 13.9 | m | β− | 0.432 | 70.0 |
| | | | | β− | 0.366 | 30.8 |
| 94 | Pu-232 | 33.8 | m | α | 6.600 | 15.0 |
| | | | | EC | 0.960 | 80.0 |
| 94 | Pu-235 | 25.3 | m | EC | 1.139 | 57.0 |
| | | | | EC | 1.105 | 37.0 |
| 94 | Pu-237 | 45.6 | d | EC | 0.220 | 79.0 |
| | | | | EC | 0.187 | 11.8 |
| 94 | Pu-243 | 5.0 | h | β− | 0.172 | 60.0 |
| | | | | β− | 0.144 | 21.0 |
| | | | | γ | 0.084 | 23.0 |
| 94 | Pu-245 | 10.5 | h | β− | 0.301 | 53.0 |
| | | | | β− | 0.111 | 15.0 |
| | | | | β− | 0.405 | 10.0 |
| | | | | γ | 0.327 | 27.0 |
| 94 | Pu-246 | 10.8 | d | β− | 0.047 | 85.0 |
| | | | | γ | 0.044 | 25.0 |
| | | | | γ | 0.224 | 23.5 |
| 95 | Am-235 | 10.3 | m | EC | 2.442 | 31.9 |
| 95 | Am-237 | 73.6 | m | EC | 1.200 | 49.7 |
| | | | | EC | 1.325 | 10.0 |
| | | | | γ | 0.280 | 47.3 |
| 95 | Am-238 | 98.0 | m | EC | 1.297 | 54.0 |
| | | | | EC | 1.655 | 14.8 |
| | | | | EC | 2.260 | 11.0 |
| | | | | γ | 0.963 | 28.0 |
| | | | | γ | 0.919 | 23.0 |
| | | | | γ | 0.561 | 10.9 |
| 95 | Am-239 | 11.9 | h | EC | 0.517 | 71.0 |
| | | | | EC | 0.290 | 18.1 |
| | | | | γ | 0.278 | 15.0 |
| | | | | γ | 0.228 | 11.3 |
| 95 | Am-240 | 50.8 | h | EC | 0.354 | 98.6 |
| | | | | γ | 0.988 | 72.2 |
| | | | | γ | 0.889 | 24.7 |
| 95 | Am-242 | 16.0 | h | β− | 0.186 | 45.0 |
| | | | | β− | 0.200 | 37.0 |
| | | | | EC | 0.707 | 10.6 |
| 95 | Am-244 | 10.1 | h | β− | 0.110 | 100.0 |
| | | | | γ | 0.744 | 66.0 |
| | | | | γ | 0.898 | 28.0 |
| | | | | γ | 0.154 | 16.0 |
| 95 | Am-244m1 | 26.0 | m | β− | 0.512 | 72.0 |
| | | | | β− | 0.496 | 25.0 |
| 95 | Am-245 | 2.1 | h | β− | 0.281 | 78.0 |
| | | | | β− | 0.193 | 15.0 |
| 95 | Am-246 | 39.0 | m | β− | 0.391 | 100.0 |
| | | | | γ | 0.679 | 64.0 |
| | | | | γ | 0.205 | 44.0 |
| | | | | γ | 0.154 | 31.0 |
| | | | | γ | 0.756 | 16.1 |
| 95 | Am-246m1 | 25.0 | m | β− | 0.429 | 37.5 |
| | | | | β− | 0.519 | 16.1 |
| | | | | β− | 0.419 | 14.2 |
| | | | | γ | 1.079 | 27.8 |
| | | | | γ | 0.799 | 24.8 |
| | | | | γ | 1.062 | 17.2 |
| | | | | γ | 1.036 | 12.7 |
| 95 | Am-247 | 23.0 | m | β− | 0.443 | 63.0 |
| | | | | β− | 0.465 | 31.0 |
| | | | | γ | 0.285 | 25.0 |
| 96 | Cm-239 | 2.7 | h | γ | 0.188 | 36.0 |
| | | | | γ | 0.146 | 11.9 |
| 96 | Cm-240 | 27.0 | d | α | 6.291 | 70.9 |
| | | | | α | 6.248 | 28.8 |
| 96 | Cm-241 | 32.8 | d | EC | 0.131 | 42.2 |
| | | | | EC | 0.115 | 26.7 |
| | | | | EC | 0.296 | 26.0 |
| | | | | γ | 0.472 | 71.0 |
| 96 | Cm-251 | 16.8 | m | β− | 0.474 | 73.0 |
| | | | | β− | 0.274 | 14.0 |
| | | | | γ | 0.543 | 11.0 |
| 97 | Bk-245 | 5.0 | d | EC | 0.558 | 90.0 |
| | | | | γ | 0.253 | 31.0 |
| 97 | Bk-246 | 1.8 | d | EC | 0.508 | 49.0 |
| | | | | EC | 0.474 | 21.0 |
| | | | | EC | 0.226 | 10.8 |
| | | | | γ | 0.799 | 62.0 |
| 97 | Bk-248 | 23.7 | h | β− | 0.267 | 45.0 |
| | | | | β− | 0.252 | 20.0 |
| 97 | Bk-250 | 3.2 | h | β− | 0.228 | 83.4 |
| | | | | γ | 0.989 | 45.0 |
| | | | | γ | 1.032 | 35.6 |
| 97 | Bk-251 | 55.6 | m | β− | 0.286 | 90.0 |
| 97 | Bk-251-m1 | 23.7 | h | EC | 0.706 | 23.0 |
| 98 | Cf-244 | 19.4 | m | α | 7.209 | 52.0 |
| | | | | α | 7.174 | 17.0 |
| 98 | Cf-245 | 45.0 | m | α | 7.138 | 32.4 |
| 98 | Cf-246 | 35.7 | h | α | 6.750 | 79.3 |
| | | | | α | 6.708 | 20.6 |
| 98 | Cf-247 | 3.1 | h | EC | 0.572 | 74.0 |
| | | | | EC | 0.530 | 21.0 |
| 98 | Cf-253 | 17.8 | d | β− | 0.065 | 50.0 |
| | | | | β− | 0.079 | 50.0 |
| 98 | Cf-255 | 85.0 | m | β− | 0.218 | 100.0 |
| 99 | Es-249 | 102.2 | m | EC | 1.070 | 39.0 |
| | | | | EC | 1.450 | 25.0 |
| | | | | EC | 0.637 | 10.0 |
| | | | | γ | 0.380 | 41.0 |
| 99 | Es-250 | 8.6 | h | EC | 0.622 | 53.2 |
| | | | | EC | 0.600 | 41.0 |
| | | | | EC | 0.642 | 10.0 |
| | | | | γ | 0.829 | 72.0 |
| | | | | γ | 0.303 | 22.0 |
| | | | | γ | 0.349 | 20.1 |
| | | | | γ | 0.384 | 13.8 |
| 99 | Es-250m1 | 2.2 | h | EC | 2.100 | 50.0 |
| | | | | EC | 1.068 | 23.2 |
| | | | | γ | 0.989 | 13.3 |
| | | | | γ | 1.032 | 10.6 |
| 99 | Es-251 | 33.0 | h | EC | 0.377 | 80.0 |
| | | | | EC | 0.199 | 22.0 |
| 99 | Es-253 | 20.5 | d | α | 6.633 | 89.9 |
| 99 | Es-254m1 | 39.3 | h | β− | 0.138 | 56.0 |
| | | | | β− | 0.361 | 25.0 |
| | | | | β− | 0.125 | 16.0 |
| | | | | γ | 0.649 | 29.0 |
| | | | | γ | 0.694 | 24.8 |
| | | | | γ | 0.689 | 12.5 |
| 99 | Es-255 | 39.8 | d | β− | 0.080 | 92.0 |
| 99 | Es-256m1 | 7.6 | h | β− | 0.075 | 86.0 |
| | | | | β− | 0.037 | 14.0 |
| | | | | γ | 0.862 | 50.3 |
| | | | | γ | 0.231 | 30.7 |
| | | | | γ | 0.173 | 24.8 |
| | | | | γ | 1.093 | 23.7 |
| | | | | γ | 0.218 | 14.6 |
| | | | | γ | 0.834 | 14.0 |
| | | | | γ | 0.106 | 13.1 |
| | | | | γ | 0.135 | 13.1 |
| 100 | Fm-250 | 30.0 | m | α | 7.435 | 75.0 |
| | | | | α | 7.396 | 15.0 |
| 100 | Fm-251 | 5.3 | h | EC | 1.432 | 82.0 |
| | | | | EC | 1.384 | 11.0 |
| 100 | Fm-252 | 25.4 | h | α | 7.039 | 84.0 |
| | | | | α | 6.998 | 15.0 |
| 100 | Fm-254 | 3.2 | h | α | 7.192 | 84.9 |
| | | | | α | 7.150 | 14.2 |
| 100 | Fm-255 | 20.1 | h | α | 7.022 | 93.4 |
| 101 | Md-255 | 27.0 | m | EC | 0.981 | 60.0 |
| | | | | EC | 0.812 | 27.0 |
| 101 | Md-256 | 77.7 | m | EC | 1.970 | 58.0 |
| | | | | EC | 0.644 | 13.0 |
| | | | | EC | 0.596 | 10.0 |
| 101 | Md-257 | 5.5 | h | α | 7.074 | 14.0 |
| | | | | EC | 0.407 | 100.0 |
| | | | | γ | 0.371 | 12.0 |
| 102 | No-259 | 58.0 | m | α | 7.505 | 75.0 |
| | | | | EC | 0.486 | 25.0 |

In an embodiment of the present invention, the radionuclide is used for diagnosis. Preferably, the radioactive isotope is selected from the group, but not limited to, comprising ⁴³Sc, ⁴⁴Sc, ⁵¹Mn, ⁵²Mn, ⁶⁴Cu, ⁶⁷Ga, ⁶⁸Ga, ⁸⁶Y, ⁸⁹Zr, ^{94m}Tc, ^{99m}Tc, ¹¹¹In, ¹⁵²Tb, ¹⁵⁵Tb, ¹⁷⁷Lu, ²⁰¹Tl, ²⁰³Pb. More preferably, the radionuclide is selected from the group comprising ⁴³Sc, ⁴⁴Sc, ⁶⁴Cu, ⁶⁷Ga, ⁶⁸Ga, ⁸⁶Y, ⁸⁹Zr, ¹¹¹In, ¹⁵²Tb, ¹⁵⁵Tb, ²⁰³Pb. Even more preferably, the radionuclide is ⁶⁴Cu, ⁶⁸Ga and ¹¹¹In. It will, however, also be acknowledged by a person skilled in the art that the use of said radionuclide is not limited to diagnostic purposes, but encompasses their use in therapy and theragnostics when conjugated to the compound of the invention.

In an embodiment of the present invention, the radionuclide is used for therapy. Preferably, the radioactive isotope is selected from the group comprising ⁴⁷Sc, ⁶⁷Cu, ⁸⁹Sr, ⁹⁰Y, ¹¹¹In, ¹⁵³Sm, ¹⁴⁹Tb, ¹⁶¹Tb, ¹⁷⁷Lu, ¹⁸⁶Re, ¹⁸⁸Re, ²¹²Pb, ²¹³Bi, ²²³Ra, ²²⁵Ac, ²²⁶Th, ²²⁷Th. More preferably, the radioactive isotope is selected from the group comprising ⁴⁷Sc, ⁶⁷Cu, ⁹⁰Y, ¹⁷⁷Lu, ²¹²Pb, ²¹³Bi, ²²⁵Ac, ²²⁷Th. Even more preferably, the radionuclide is selected from the group comprising ⁹⁰Y, ¹⁷⁷Lu, ²²⁵Ac and ²²⁷Th. It will, however, also be acknowledged by a person skilled in the art that the use of said radionuclide is not limited to therapeutic purposes, but encompasses their use in diagnostic and theragnostics when conjugated to the compound of the invention.

In an embodiment, the compound of the invention comprises a chelator. Preferably, the chelator is part of the compound of the invention, whereby the chelator is either directly or indirectly such as by a linker attached to the compound of the invention. A preferred chelator is a chelator which forms metal chelates preferably comprising at least one radioactive metal. The at least one radioactive metal is preferably useful in or suitable for diagnostic and/or therapeutic and/or theragnostic use and is more preferably useful in or suitable for imaging and/or radiotherapy.

Chelators in principle useful in and/or suitable for the practicing of the instant invention including diagnosis and/or therapy of a disease are known to the person skilled in the art. A wide variety of respective chelators is available and has been reviewed, e.g. by Banerjee et al. (Banerjee, et al., Dalton Trans, 2005, 24: 3886), and references therein (Price, et al., Chem Soc Rev, 2014, 43: 260; Wadas, et al., Chem Rev, 2010, 110: 2858). Such chelators include, but are not limited to linear, cyclic, macrocyclic, tetrapyridine, N3S, N2S2 and N4 chelators as disclosed in U.S. Pat. Nos. 5,367,080 A, 5,364,613 A, 5,021,556 A, 5,075,099 A and 5,886,142 A.

Representative chelators and their derivatives include, but are not limited to AAZTA, BAT, CDTA, DTA, DTPA, CY-DTA, DTCBP, CTA, cyclam, cyclen, TETA, sarcophagine, CPTA, TEAMA, Cyclen, DO3A, DO2A, TRITA, DATA, DFO, DATA(M), DATA(P), DATA(Ph), DATA(PPh), DEDPA, H4octapa, H2dedpa, H5decapa, H2azapa, H2CHX-DEDPA, DFO-Chx-MAL, DFO-p-SCN, DFO-1AC, DFO-BAC, p-SCN-Bn-DFO, DFO-pPhe-NCS, DFO-HOPO, DFC, diphosphine, DOTA, DOTAGA, DOTA-MFCO, DOTAM-mono-acid, nitro-DOTA, nitro-PA-DOTA, p-NCS-Bz-DOTA, PA-DOTA, DOTA-NCS, DOTA-NHS, CB-DO2A, PCTA, p-NH₂-Bn-PCTA, p-SCN-Bn-PCTA, p-SCN-Bn-DOTA, DOTMA, NB-DOTA, H4NB-DOTA, H4TCE-DOTA, HOPO, 2,3-HOPO, 3,4,3-(Li-1,2-HOPO), TREN(Me-3,2-HOPO), TCE-DOTA, DOTP, DOXP, p-NCS-DOTA, p-NCS-TRITA, TRITA, TETA, 3p-C-DEPA, 3p-C-DEPA-NCS, p-NH2-BN—OXO-DO3A, p-SCN-BN-TCMC, TCMC, 4-aminobutyl-DOTA, azido-mono-amide-DOTA, BCN-DOTA, butyne-DOTA, BCN-DOTA-GA, DOA3P, DO2a2p, DO2A(trans-H2do2a), H2DO2A, H2ODO2A, DO3A, DO3A-thiol, DO3AM-acetic acid, DO2AP, CB-DO2A, C3B-DO2A, HP-DO3A, DOTA-NHS-ester, maleimide-DOTA-GA, maleimido-mono-aminde-DOTA, maleimide-DOTA, NH₂-DOTA-GA, NH₂-PEG4-DOTA-GA, GA, p-NH₂—Bn-DOTA, p-NO₂—Bn-DOTA, p-SCN-Bn-DOTA, p-SCN-Bz-DOTA, TA-DOTA, TA-DOTA-GA, OTTA, DOXP, TSC, DTC, DTCBP, PTSM, ATSM, H2ATSM, H2PTSM, Dp44mT, DpC, Bp44mT, QT, hybrid thiosemicarbazone-benzothiazole, thiosemicarbazone-styrylpyridine tetradentate ligands H₂L²⁻⁴, HBED, HBED-CC, dmHBED, dmEHPG, HBED-nn, SHBED, Br-Me2HBED, BPCA, HEHA, BF-HEHA, deferiprone, THP, HYNIC (2-hydrazino nicotinamide), NHS—HYNIC, HYNIC-Kp-DPPB, HYNIC-Ko-DPPB, (HYNIC)(tricine)₂, (HYNIC)(EDDA), p-EDDHA, AIM, AIM A, IAM B, MAMA, MAMA-DGal, MAMA-MGal, MAMA-DA, MAMA-HAD, macropa, macropaquin, macroquin-SO₃, NxS4-x, N2S2, N3S, N4, MAG3, NOTA, NODAGA, SCN-Bz-NOTA-R, NOT-P (NOTMP), MA-NOTMP, NOTAM, p-NCS-NOTA, TACN, TACN-TM, NETA, NETA-monoamine, p-SCN-PhPr-NE3TA, C-NE3TA-NCS, C-NETA-NCS, 3p-C-NETA, NODASA, NOPO, NODA, NO2A, N-benzyl-NODA, C-NOTA, BCNOT-monoamine, maleimido-mono-amide-NOTA, NO2A-azide, NO2A-butyne, NO2AP, NO3AP, N-NOTA, oxo-DO3A, p-NH₂—Bn-NOTA, p-NH₂-Bn-oxo-DO3A, p-NO₂-Bn-cyclen, p-SCN-Bn-NOTA, p-SCN-Bn-oxo-DO3A, TRAP, PEPA, BF-PEPA, pycup, pycup2A, pycup1A1Bn, pycup2Bn, SarAr-R, DiAmSar, AmBaSar-R, siamSar, Sar, Tachpyr, tachpyr-(6-Me), TAM A, TAM B, TAME, TAME-Hex, THP-Ph-NCS, THP-NCS, THP-TATE, NTP, H3THP, THPN, CB-TE2A, PCB-TE1A1P, TETA-NHS, CPTA, CPTA-NHS, CB-TE1A1P, CB-TE1K1P, CB-TE2A, TE2A, H2CB-TE2A, TE2P, CB-TE2P, MM-TE2A, DM-TE2A, 2C-TETA, 6C-TETA, BAT, BAT-6, NHS-BAT ester, SSBAT, SCN-CHX-A-DTPA-P, SCN-TETA, TMT-amine, p-BZ-HTCPP, DCMC, DEPA, H2ATSM, PCBA, PIH, PSC wherein
2,3-HOPO stands for 3-hydroxypyridin-2-one,
2C-TETA stands for [4,8,11-tris-carboxymethyl-12-(4-isothiocyanato-benzyl)-1,4,8,11tetraaza-cyclotetradec-1-yl]-acetic acid,
3p-C-DEPA stands for 2-[(carboxymethyl)][5-(4-nitrophenyl-1-[4,7,10-tris(carboxymethyl)-1,4,7,10-tetraazacyclododecan-1-yl]pentan-2-yl)amino]acetic acid,
3p-C-DEPA-NCS stands for 2-[(carboxymethyl)][5-(4-thiocyanatophenyl-1-[4,7,10-tris(carboxymethyl)-1,4,7,10-tetraazacyclododecan-1-yl]pentan-2-yl)amino]acetic acid,
3p-C-NE3TA-NCS stands for {4-[2-(bis-carboxymethylamino)-5-(4-isothiocyanatophenyl) pentyl]-7-carboxymethyl[1,4,7]triazonan-1-yl}acetic acid,
3p-C-NETA stands for {4-[2-(bis-carboxymethylamino)-5-(4-nitrophenyl) pentyl]-7-carboxymethyl[1,4,7]triazonan-1-yl}acetic acid,
4-aminobutyl-DOTA stands for 1,4,7,10-tetraazacyclododecane-1,4,7-tris(acetic acid)-10-(4-aminobutyl)acetamide,
^{99m}Tc(CO)₃-chelators stands for bi- or tridendate chelators capable of forming stable complexes with technetium tricarbonyl fragments,
AAZTA stands for 6-amino-6-methylperhydro-1,4-diazepine-N,N′,N″,N″-tetraacetic acid,
AmBaSar stands for 4-((8-amino-3,6,10,13,16,19-hexaazabicyclo [6.6.6] icosane-1-ylamino) methyl) benzoic acid,
ATSM stands for diacetyl-bis(N4-methylthiosemicarbazone),
azido-mono-amide-DOTA stands for 1,4,7,10-tetraazacyclododecane-1,4,7-tris(acetic acid)-10-(azidopropyl ethylacetamide),
BAT stands for 3,15,27-triamino-7,19,31-trihydroxy-10,22,34-trimethyl-1,13,25-trioxa-7,19,31-triaza-cyclohexatriaconta-9,21,33-triene-2,8,14,20,26,32-hexaone,
BCN-DOTA-GA stands for 2,2′,2″-(10-(4-((2-((((1R,8S,9s)-bicyclo[6.1.0]non-4-yn-9-ylmethoxy)carbonyl)amino)ethyl)amino)-1-carboxy-4-oxobutyl)-1,4,7,10-tetraazacyclododecane-1,4,7-triyl)triacetic acid,
BF-HEHA stands for 3-(4-isothicyanatobenzyl)-1,2,7,10,13-hexaazacyclooctadecane-1,4,7,10,13,16-hexaacetic acid,
BF-PEPA stands for 2-(4-thiocyanatobenzoyl)-1, 4, 7, 10, 13-pentaazacyclopentadecane-N, N′, N″, N′″, N″″-pentaacetic acid,
Bp44mT stands for 2-benzoylpyridine-4,4-dimethyl-3-thiosemicarbazone,
BPCA stands for bipyridine-chelator,
Br-Me2HBED stands for N-(2-hydroxy-3,5-dimethylbenzyl)-Ar′-(2-hydroxy-5-(bromoacetamido)benzyl)ethylenediamine-N,—N′-diacetic acid,
butyne-DOTA stands for 1,4,7,10-tetraazacyclododecane-1,4,7-tris(acetic acid)-10-(3-butynylacetamide),
CB-DO2A stands for 4,10-bis(carboxymethyl)-1,4,7,10-tetraazabicyclo[5.5.2]tetradecane,
CB-TE1A1P stands for (1,8-diamino-3,6,10,13,16,19-hexaazabicyclo[6.6.6]icosane),
CB-TE1K1P stands for 6-amino-2-(11-phosphonomethyl-1,4,8,11-tetraaza-bicyclo[6.6.2]hexadec-4-yl)-hexanoic acid,
CB-TE2A stands for 4,11-bis-(carboxymethyl)-1,4,8,11-tetraazabicyclo[6.6.2]-hexadecane,
CB-TE2P stands for 1,4,8,11-tetraazacyclotetradecane-1,8-di(methanephosphonic acid),
CDTA stands for trans-1,2-diaminocyclohexane-N,N,N′,N′-tetraacetic acid,
CHX-A″-DTPA stands for [(2-{[2-(bis-carboxymethyl-amino)-cyclohexyl]-carboxymethyl-amino}-ethyl)-carboxymethyl-amino]-acetic acid,
C-NOTA stands for [4,7-bis-carboxymethyl-2-(4-nitro-benzyl)-[1,4,7]triazonan-1-yl]-acetic acid,
CPTA stands for 4-((1,4,8,11-tetraazacyclotetradecan-1-yl)methyl)benzoic acid,
cyclam stands for 1,4,8,11-tetraazacyclotetradecane,
cyclen stands for 1,4,7,10-tetraazacyclododecane,
CY-DTA stands for trans-1,2-diaminocyclohexane-N,N,N′,N′-tetraacetic acid, monohydrate,
DATA stands for [4-carboxymethyl-6-(carboxymethyl-methyl-amino)-6-methyl-[1,4]diazepan-1-yl]-acetic acid,
DCMC stands for 1,7-bis(carbamoylmethyl)-1,4,7,10-tetraazacyclodocane,
deferiprone (also called DMHP, CP20, L1) stands for 3-hydroxy-1,2-dimethyl-4(1H)-pyridone,
DEPA stands for 7-[2-(bis-carboxymethylamino)-ethyl]-4,10-bis-carboxymethyl-1,4,7,10-tetraaza-cyclododec-1-yl-acetic acid,
DEPA stands for diethylenetriamine pentaacetic acid,
DFO stands for the Desferal or Desferrioxamine type group of chelators, the chemical name of the non-limiting example is N-[5-({3-[5-(Acetyl-hydroxy-amino)-pentylcarbamoyl]-propionyl}-hydroxy-amino)-pentyl]-N′-(5-amino-pentyl)-N′-hydroxy-succinamide,
DFO-BAC stands for bromoacetyl-desferrioxamine,
DFO-HOPO stands for N1-hydroxy-N1-(5-(4-(hydroxy(5-(1-hydroxy-6-oxo-1,6-dihydropyridine-2-carboxamido)pentyl)amino)-4-oxobutanamido)pentyl)-N4-(5-(Nhydroxyacetamido)pentyl)succinamide,
DFO-pPhe-NCS(=p-SCN-Bn-DFO) stands for 1-(4-isothiocyanatophenyl)-3-[6,17-dihydroxy-7,10,18,21-tetraoxo-27-(N-acetylhydroxylamino)-6,11,17, 22-tetraazaheptaeicosine]thiourea,
DiAmSar stands for 1,8-diamino-3,6,10,13,16,19-hexaazabicyclo[6.6.6]icosane,
dmEHPG stands for N,N-8-ethylene-bis(o-hydroxyphenylglycine) dimethyl ester,
dmHBED stands for N,N*-bis(o-hydroxybenzyl) ethylenediamine diacetic acid,
DM-TE2A stands for 1,8-N,N′-bis-(carboxymethyl)-4,11-N″,N′″-bis-(methyl)-1,4,8,11-tetraazacyclotetra decane,
DO2A stands for 1,4,7,10-tetraazacyclododecane-1,7-diacetic acid,
DO2AP stands for 4-[phosphorylmethyl]-1,4,7,10-tetrazacyclododecane-1,7-diacetic acid,
DO2a2p stands for 1,4,7,10-tetraazacyclododecane-1,7-bis(acetic acid)-4,10-bis(methylenephosphonic acid),
DO3A stands for 1,4,7,10-tetraazacyclododecane-1,4,7-triacetic acid,
DO3AM-acetic acid stands for 2-(4,7,10-tris(2-amino-2-oxoethyl)-1,4,7,10-tetraazacyclododecan-1-yl)acetic acid,
DO3AP stands for 7-[phosphorylmethyl]-1,4,7,10-tetrazacyclododecane-1,4,10-triacetic acid,
DO3A-thiol stands for 1,4,7,10-tetraazacyclododecane-1,4,7-tris(acetic acid)-10-(2-thioethyl)acetamide,
DOTA (also called tetraxetan) stands for 1,4,7,10-tetrazacyclododecane-1,4,7,10-tetraacetic acid,
DOTAGA stands for 1,4,7,10-tetraazacyclodocecane,1-(glutaric acid)-4,7,10-triacetic acid,
DOTAM (also called TCMC) stands for 1,4,7,10-tetrakis[carbamoylmethyl]-1,4,7,10-tetracyclodecane,
DOTAM-mono-acid stands for 1,4,7,10-tetraazacyclododecane-1,4,7-tri(carbamoylmethyl)-10-acetic acid,
DOTA-NCS stands for [4,7,10-tris-carboxymethyl-6-(4-isothiocyanato-benzyl)-1,4,7,10tetraaza-cyclododec-1-yl]-acetic acid,
DOTA-NHS stands for [4,10-bis-carboxymethyl-7-(2,5-dioxo-pyrrolidin-1-yloxycarbonylmethyl)-1,4,7,1Otetraaza-cyclododec-1-yl]-acetic acid,
DOTA-NHS-ester stands for 2,2′,2″-(10-(2-(2,5-dioxopyrrolidin-1-yl)oxy)-2-oxoethyl)-1,4,7,10-tetraazacyclododecane-1,4,7-triyl)triacetic acid,
DOTMA stands for 1,4,7,10-tetraazacyclododecane-1,4,7,10-tetra(methylene phosphonic acid),
DOTP stands for 1,4,7,10-tetraazacyclododecane-1,4,7,10-tetra(methylene phosphonic acid),
DOXP stands for (di-2-pyridylketone-4,4-dimethyl-3-thiosemicarbazone,
Dp44mT stands for 2-(di-2-pyridinylmethylene)-N,N-dimethyl-hydrazinecarbothioamide, di-2-pyridylketone-4,4,-dimethyl-3-thiosemicarbazone,
DpC stands for di-2-pyridylketone-4-cyclohexyl-4-methyl-3-thiosemicarbazone,
DTC stands for diethyldithiocarbamate,
DTPA stands for diethylenetriaminepentaacetic acid,
EDDA stands for ethylenediaminediacetic acid,
FSC (also called fusarine C) stands for 3,15,27-triamino-7,19,31-trihydroxy-10,22,34-trimethyl-1,13,25-trioxa-7,19,31-triaza-cyclohexatriaconta-9,21,33-triene-2,8,14,20,26,32-hexaone,
H2ATSM stands for 1,4,8,11-tetraazacyclotetradecane-1-(methanephosphonic acid)-8-(methanecarboxylic acid),
H2CB-TE2A stands for 4,11-bis(carboxymethyl)-1,4,8,11-tetraazabicyclo[6.6.2]hexadecane,
H2CHX-DEDPA stands for N,N′-(6-carboxy-2-pyridylmethyl)-N,N′-diacetic acid-1,2-diaminoethane,
H2dedpa stands for 1,2-[{6-(carboxylato)pyridin-2-yl}methylamino]ethane,
H2DO2A stands for 1,4,7,10-tetraazacyclododecane-1,7-diacetic acid,
H2ODO2A stands for 1-oxa-4,7,10-triazacyclododecane-4,10-diacetic acid,
H2PTSM stands for pyruvaldehyde bis(methy-lthiosemicarbazone),
H4octapa stands for N,N′-(6-carboxy-2-pyridylmethyl)-N,N′-diacetic acid-1,2-diaminoethane,
HBED stands for bis(2-hydroxybenzyl) ethylenediaminediacetic acid,
HBED-CC stands for N,N′-bis[2-hydroxy-5-(carboxyethyl)benzyl]ethylenediamine-N,N′-diacetic acid,
HEHA stands for 1,4,7,10,13,16-hexaazacyclooctadecane-N,N′,N″,N′,N″″,N′″″-hexaacetic acid,
HOPO stands for the octadentate hydroxypyridinone-type group of chelators,
HP-DO3A stands for 2,2′,2″-[10-(2-hydroxypropyl)-1,4,7,10-tetraazacyclododecane-1,4,7-triyl]triacetic acid,
HYNIC stands for 6-hydrazino-nicotinic acid,
HYNIC-Ko-DPPB stands for N-ε-(2-(diphenylphosphino)benzoyl)-N-α-(6-(2-(2-Sulfonatobenzaldehyde)hydrazono)nicotinyl)lysine methyl ester,
HYNIC-Kp-DPPB stands for N-ε-(4-(diphenylphosphino)benzoyl)-N-α-(6-(2-(2-sulfonatobenzaldehyde)hydrazono)nicotinyl)lysine methyl ester,
macropa stands for N,N′-bis[(6-carboxy-2-pyridyl)methyl]-4,13-diaza-18-crown,
MAG3 stands for (N-hydroxysuccinimidyl S-acetylmercaptoacetyltriglycinate,
maleimide-DOTA stands for 2,2′,2″-(10-(1-carboxy-4-((2-(2,5-dioxo-2,5-dihydro-1H-pyrrol-1-yl)ethyl)amino)-4-oxobutyl)-1,4,7,10-tetraazacyclododecane-1,4,7-triyl)triacetic acid,
maleimide-DOTA-GA stands for 2,2′,2″-(10-(1-carboxy-4-((2-(2,5-dioxo-2,5-dihydro-1H-pyrrol-1-yl)ethyl)amino)-4-oxobutyl)-1,4,7,10-tetraazacyclododecane-1,4,7-triyl)triacetic acid,
maleimido-mono-amide-NOTA stands for 1,4,7-triazacyclononane-1,4-bis-acetic acid-7-maleimidoethylacetamide,
maleimido-mono-amine-DOTA stands for 1,4,7,10-tetraazacyclododecane-1,4,7-tris-acetic acid-10-maleimidoethylacetamide,
MAMA stands for monoamine-monoamide dithiol,
MAMA-DA stands for N-[[[(2-mercaptoethyl)amino]carbonyl]methyl]-N-(2-mercaptoethyl)-6-aminododecanoic acid,
MAMA-HA stands for N-[[[(2-mercaptoethyl)amino]carbonyl]methyl]-N-(2-mercaptoethyl)-6-aminohexanoic acid,
MAMA-HAD stands for N-[[[(2-mercaptoethyl)amino]carbonyl]methyl]-N-(2-mercaptoethyl)-6-aminohexadecanoic acid,
MA-NOTMP stands for methylaminotriazacyclononane trimethylphosphinate,
MM-TE2A stands for 1,8-N,N′-bis-(carboxymethyl)-4-N″-(methyl)-1,4,8,11-tetraazacyclotetradecane,
N2S2 stands for N,N′-bis-(2-amino-ethyl)-propane-1,3-diamine,
N3OA stands for 4,7,10-tris(carbamoylmethyl)-,4,7,10-triaza-12-crown-ether,
N3S stands for (sulfanylidenehydrazinylidene)azanide,
N4 stands for N,N′-bis-(2-amino-ethyl)-propane-1,3-diamine,
NB-DOTA stands for {4-[2-(bis-carboxymethyl-amino)-ethyl]-7-carboxymethyl-[1,4,7]triazonan-1-yl}-acetic acid,
N-benzyl-NODA stands for 1-benzyl-1,4,7-triazonane-1,4-diyl)diacetic acid,
NE3TA stands for {4-carboxymethyl-7-[2-(carboxymethyl-amino)-ethyl]-[1,4,7]triazonan-1-yl}-acetic acid,
NETA stands for {4-[2-(bis-carboxymethyl-amino)-ethyl]-7-carboxymethyl-[1,4,7]triazonan-1-yl}-acetic acid,
NH2-DOTA-GA stands for 2,2′,2″-(10-(4-((2-aminoethyl)amino)-1-carboxy-4-oxobutyl)-1,4,7,10-tetraazacyclododecane-1,4,7-triyl)triacetic acid,
NH2-PEG4-DOTA-GA stands for 2,2′,2″-(10-(1-amino-19-carboxy-16-oxo-3,6,9,12-tetraoxa-15-azanonadecan-19-yl)-1,4,7,10-tetraazacyclododecane-1,4,7-triyl)triacetic acid,
NHS-BAT ester stands for N-hydroxysuccinimide ester of 6-(4′-(4″-carboxyphenoxy)butyl)-2, 10-dimercapto-2, 10-dimethyl-4,8-diazaundecane,
NHS-HYNIC stands for N-hydroxysuccinimidyl hydrazino nicotinate hydrochloride,
nitro-DOTA stands for 2-(g-nitrobenzyl)-1,4,7,10-tetraazacyclododecane-N,N′,N″,N′″-tetraacetic acid,
nitro-PA-DOTA stands for a-(2-(g-nitrophenyl)ethyl)-1,4,7,10-tetraazacyclododecane-1-acetic-4,7,10-tris(methylacetic) acid,
NO2A stands for 1,4,7-triazacyclononane-N,N′,N″-triacetic acid,
NO2A-azide stands for 1,4,7-triazacyclononane-1,4-bis(acetic acid)-7-(3-azidopropylacetamide),
NO2A-butyne stands for 1,4,7-triazacyclononane-1,4-bis(acetic acid)-7-(3-butynylacetamide),
NO2AP stands for triazacyclononane,
NO3AP stands for 1,4,7-triazacyclononane-N-glutaric acid-N′,N″-diacetic acid,
NODA stands for 4,10-bis(carbamoylmethyl)-4,10-diaza-12-crown-ether,
NODAGA stands for 1,4,7-triazacyclononane-N-glutaric acid-N′,N″-diacetic acid,
NODA-MPAA stands for 1,4,7-triazacyclononane-1,4-diacetate-methyl phenylacetic acid,
NOPO stands for 3-(((4,7-bis((hydroxy(hydroxymethyl)phosphoryl)methyl)-1,4,7-triazonan-1-yl)methyl)(hydroxy)phosphoryl)propanoic acid,
NOTA stands for 1,4,7-triazacyclononanetriacetic acid,
NOTAM stands for 2,2′,2″-(1,4,7-triazacyclononane-1,4,7-triyl)triacetamide,
NOTA-NHS stands for 3-hydroxy-2-oxopyridine,
NOTA-NHS ester stands for 2-(4,7,10-tris(2-(tert-butoxy)-2-oxoethyl)-1,4,7,10-tetraazacyclododecan-1-yl)acetic acid,
NOTP stands for 1,4,7-triazacyclononane-N,N′N″-tris(methylene phosphonic) acid),
NTP stands for {4-carboxymethyl-7-[2-(carboxymethyl-amino)-ethyl]-[1,4,7]triazonan-1-yl}-acetic acid,
NxS4-x (N4, N2S2, N3S) stands for a group of tetradentate chelators with N-atoms (basic amine or non-basic amide) and thiols as donors stabilizing Tc-complexes, especially Tc(V)-oxo complexes,
o,p-EDDHA stands for ethylenediamine-N(o-hydroxyphenylacetic)-N′(p-hydroxyphenylacetic) acid,
OPTT stands for 9-oxa-3,6,12,15,21-pentaazatricyclo[15,3,2,1]trieicos-1(21),17,19-triene-2,7,11,16-tetradione,
OTTA stands for 1-oxa-4,7,10-triazacyclododecane-N,N′,N″-triacetic acid,
oxo-DO3A stands for 1-oxa-4,7,10-triazacyclododecane-4,7,10-triacetic acid,
PA-DOTA stands for 1,4,7,10-tetraaza-N-(1-carboxy-3-(4-nitrophenyl)propyl)-N′,N″,N′″-tris(acetic acid)cyclododecane,
p-BZ-HTCPP stands for 3,6,9,15-tetraazabicyclo[9.3.1]-pentadeca-1(15),11,13-triene-3,6,9,-triacetic acid,
PCBA stands for 1-[(1,4,7,10,13-pentaazacyclopentadec-1-yl)methyl]benzoic acid,
PCB-TE1A1P stands for 2-(11-(phosphonomethyl)-1,4,8,11-tetraazabicyclo[6.6.3]heptadecan-4-yl)acetic acid,
PCTA stands for 3,6,9,15-tetraazabicyclo[9.3.1]-pentadeca-1(15),11,13-triene-3,6,9,-triacetic acid,
PEPA stands for 1,4,7,10,13-pentaazacyclopentadecane-N, N′, N″, N′″, N″″-pentaacetic acid,
PIH stands for pyridoxal isonicotinoyl hydrazone,
PSC stands for 2,2′-(4,10-bis(2-amino-2-oxoethyl)-1,4,7,10-tetraazacyclododecane-1,7-diyl)diacetic acid or Pb specific chelator,
p-NCS-Bz-DFO stands for N1-hydroxy-N1-(5-(4-(hydroxy(5-(3-(4-thiocyanatobenzoyl)thioureido)pentyl)amino)-4-oxobutanamido)pentyl)-N4-(5-(N-hydroxyacetamido)pentyl)succinamide,
p-NCS-Bz-DOTA stands for S-2-(4-thiocyanatobenzoyl)-1,4,7,10-tetraazacyclododecane tetraacetic acid,
p-NH₂—Bn-DOTA stands for S-2-(4-aminobenzyl)-1,4,7,10-tetraazacyclododecane tetraacetic acid,
p-NH2-Bn-NOTA stands for 2-S-(4-aminobenzyl)-1,4,7-triazacyclononane-1,4,7-triacetic acid,
p-NH₂-Bn-oxo-DO3A stands for 1-oxa-4,7,10-tetraazacyclododecane-5-S-(4-aminobenzyl)-4,7,10-triacetic acid,
p-NH2-Bn-PCTA stands for 3,6,9,15-tetraazabicyclo[9.3.1]pentadeca-1(15),11,13-triene-4-S-(4-aminobenzyl)-3,6,9-triacetic acid,
p-NO2-Bn-cyclen stands for S-2-(4-nitrobenzyl)-1,4,7,10-tetraazacyclododecane,
p-NO2-Bn-DOTA stands for S-2-(4-nitrobenzyl)-1,4,7,10-tetraazacyclododecane tetraacetic acid,
p-SCN-Bn-DFO stands for N1-hydroxy-N1-(5-(4-(hydroxy(5-(3-(4-isothiocyanatophenyl)thioureido)pentyl)amino)-4-oxobutanamido)pentyl)-N4-(5-(N-hydroxyacetamido)pentyl)succinamide,
p-SCN-Bn-DOTA stands for S-2-(4-isothiocyanatobenzyl)-1,4,7,10-tetraazacyclododecane tetraacetic acid,
p-SCN-Bn-NOTA stands for 2-S-(4-isothiocyanatobenzyl)-1,4,7-triazacyclononane-1,4,7-triacetic acid,
p-SCN-Bn-oxo-DO3A stands for 1-oxa-4,7,10-tetraazacyclododecane-5-S-(4-isothiocyanatoobenzyl)-4,7,10-triacetic acid,
p-SCN-Bn-PCTA stands for 3,6,9,15-tetraazabicyclo[9.3.1]pentadeca-1(15),11,13-triene-4-S-(4-isothiocyanatobenzyl)-3,6,9-triacetic acid,
p-SCN-BN-TCMC stands for S-2-(4-isothiocyanatobenzyl)-1,4,7,10-tetraaza-1,4,7,10-tetra(2-carbamoylmethyl)cyclododecane,
p-SCN-Bz-DOTA stands for S-2-(4-isothiocyanatobenzoyl)-1,4,7,10-tetraazacyclododecane tetraacetic acid,
p-SCN-Bz-NOTA stands for 2-S-(4-isothiocyanatobenzoyl)-1,4,7-triazacyclononane-1,4,7-triacetic acid,
p-SCN-PhPr-NE3TA stands for 2,2′-(7-(2-((carboxymethyl)(3-(4-isothiocyanatophenyl)propyl)amino)ethyl)-1,4,7-triazonane-1,4-diyl)diacetic acid,
PTSM stands for pyruvaldehyde bis(N(4)-methylthiosemicarbazone),
pycup stands for 1,8-(2,6-pyridinedimethylene)-1,4,8,11-tetraazacyclo-tetradecane,
pycup2Bn stands for N1-hydroxy-N1-(5-(4-(hydroxy(5-(3-(4-isothiocyanatophenyl)thioureido)pentyl)amino)-4-oxobutanamido)pentyl)-N4-(5-(N-hydroxyacetamido)pentyl)succinamide,
Sar (also called Sarcophagine) stands for 3,6,10,13,16,19-hexaazabicyclo[6.6.6]icosane,
SarAr stands for (1-N-(4-aminobenzyl)-3, 6,10,13,16,19-hexaazabicyclo[6.6.6] eicosane-1,8-diamine),
SCN-CHX-A-DTPA-P stands for [(R)-2-amino-3-(4-isothiocyanatophenyl)propyl]-trans-(S,S)-cyclohexane-1,2-diamine-pentaacetic acid,
SCN-TETA stands for 6-[p-(isothiocyanato)benzyl]-1,4,8, 11-tetraazacyclotetradecane-1,4,8,11-tetraacetic acid,
SHBED stands for 3,6,10,13,16,19-hexaazabicyclo(6,6,6)icosane,
Tachpyr stands for (N,N′N″-tris(2-pyridylmethyl)-cis,cis-1,3,5-triaminocyclohexane),
tachpyr-(5-Me) stands for 1,3,5-cis,cis,-triaminocyclohexane-N,N′,N″-tri-(5-methyl-2-methylpyridineimine),
TACN stands for 1,4,7-triazacyclononane,
TACN-TM stands for 1,4,7-tris (2-mercaptoethyl)-1,4,7-triazacyclononane,
TA-DOTA stands for 2,2′,2″-(10-(2-((2-(5-(1,2-dithiolan-3-yl)pentanamido)ethyl)amino)-2-oxoethyl)-1,4,7,10-tetraazacyclododecane-1,4,7-triyl)triacetic acid,
TA-DOTA-GA stands for 1,1,1-tris(aminomethyl)ethane,
TAM A stands for methyl-(2-methyl-3-methylamino-2-methylaminomethyl-propyl)-amine,
TAME stands for 1,1,1-tris-(aminomethyl)ethane,
TAME-Hex stands for (1,8-N,N′-bis(carboxymethyl)-1,4,8,11-tetraazacyclotetradecane,
TBPD stands for 3,6,9,15-tetraazabicyclo[9.3.1]pentadeca-1(15),11,13-triene-2,10-dione,
TCMC stands for 1,4,8,11-tetraazacyclotetradecane-1,4,8,11-tetraacetic acid,
TE2A stands for [4,8-bis-carboxymethyl-11-(2,5-dioxo-3-sulfo-pyrrolidin-1-yloxycarbonylmethyl)-1,4,8,11tetraaza-cyclotetradec-1-yl]-acetic acid,
TEAMA stands for 6-(p-bromoacetamidobenzyl)-1,4,8,11-tetraazacyclotetradecane-1,4,8,11-tetraacetic acid,
TETA stands for 1,4,8,11-tetraazacyclododecane-1,4,8,11-tetraacetic acid,
TETAM stands for 2,2′,2″,2′″-(1,4,8,11-tetraazacyclotetradecane-1,4,8,11-tetrayl)tetraacetamide,
THP stands for hexadentate tris(3,4-hydroxypyridinone),
THPN stands for 1,3-propanediamine-N,N,N′,N′-tetrakis[(2-(aminomethyl)-3-hydroxy-1,6-dimethyl-4(1H)-pyridinone)acetamide],
THP-TATE stands for 3,3′,3″-(((1,4,7-triazonane-1,4,7-triyl)tris(methylene))tris(hydroxyphosphoryl))tripropanoic acid,
TRAP stands for 3-({4,7-bis-[(2-carboxy-ethyl)-hydroxy-phosphinoylmethyl]-[1,4,7]triazonan-1-ylmethyl}-hydroxy-phosphinoyl)-propionic acid,
Triapine stands for dipyridyl thiosemicarbazone,
Tricine stands for picolylaminediacetic acid,
TRITA stands for 2,2′,2″,2′″-(1,4,7,10-tetraazacyclotridecane-1,4,7,10-tetrayl)tetraacetic acid,
TRITRAM stands for 2,2′,2″-(1,4,7,10-tetraazacyclotridecane-1,4,7-triyl)triacetamide·HYNIC, DTPA, EDTA, DOTA, TETA, bisamino bisthiol (BAT)-based chelators as disclosed in U.S. Pat. No. 5,720,934; desferrioxamine (DFO) as disclosed in Doulias et al. (Doulias, et al., Free Radic Biol Med, 2003, 35: 719), tetrapyridine and N3S, N2S2 and N4 chelators as disclosed in U.S. Pat. Nos. 5,367,080 A, 5,364,613 A, 5,021,556 A, 5,075,099 A, 5,886,142 A, whereby all of the references are included herein by reference in their entirety. 6-amino-6-methylperhydro-1,4-diazepine-N,N′,N″,N″-tetraacetic acid (AAZTA) is disclosed in Pfister et al. (Pfister, et al., EJNMMI Res, 2015, 5: 74), deferiprone, a 1,2-dimethyl-3,4-hydroxypyridinone and hexadentate tris(3,4-hydroxypyridinone) (THP) are disclosed in Cusnir et al. (Cusnir, et al., Int J Mol Sci, 2017, 18), monoamine-monoamide dithiol (MAMA)-based chelators are disclosed in Demoin et al. (Demoin, et al., Nucl Med Biol, 2016, 43: 802), macropa and analogs are disclosed in Thiele et al. (Thiele, et al., Angew Chem Int Ed Engl, 2017, 56: 14712), 1,4,7,10,13,16-hexaazacyclohexadecane-N,N′,N″,N′″,N″″,N′″″-hexaacetic acid (HEHA) and PEPA analogs are disclosed in Price and Orvig (Price, et al., Chem Soc Rev, 2014, 43: 260), pycup and analogs are disclosed in Boros et al. (Boros, et al., Mol Pharm, 2014, 11: 617), N, N-bis(2-hydroxybenzyl)ethylenediamine-N,N-diacetic acid (HBED), 1,4,7,10-tetrakis (carbamoylmethyl)-1,4,7,10-tetraazacyclododecane (TCM), 2-[(carboxymethyl)]-[5-(4-nitrophenyl-1-[4,7,10-tris-(carboxymethyl)-1,4,7,10-tetraazacyclododecan-1-yl]pentan-2-yl)-amino]acetic acid (3p-C-DEPA), CB-TE2A, TE2A, TE1A1P, DiAmSar, 1-N-(4-aminobenzyl)-3,6,10,13,16,19-hexaazabicyclo[6.6.6]-eicosane-1,8-diamine (SarAr), NETA, tris(2-mercaptoethyl)-1,4,7-triazacyclononane (TACN-TM), {4-[2-(bis-carboxymethyl-amino)-ethyl]-7-carboxymethyl-[1,4,7]triazonan-1-yl}-acetic acid (NETA), diethylenetriaminepentaacetic acid (DTP), 3-({4,7-bis-[(2-carboxy-ethyl)-hydroxy-phosphinoylmethyl]-[1,4,7]triazonan-1-ylmethyl}-hydroxy-phosphinoyl)-propionic acid (TRAP), NOPO, H4octapa, SHBED, BPCA, 3,6,9,15-tetraazabicyclo[9.3.1]-pentadeca-1(15),11,13-triene-3,6,9,-triacetic acid (PCTA), and 1,4,7,10,13-pentaazacyclopentadecane-N,N′,N″,N′,N″″-pentaacetic acid (PEPA) are disclosed in Price and Orvig (Price, et al., Chem Soc Rev, 2014, 43: 260), 1-hydroxy-2-pyridone ligand (HOPO) is disclosed in Allott et al. (Allott, et al., Chem Commun (Camb), 2017, 53: 8529), [4-carboxymethyl-6-(carboxymethyl-methyl-amino)-6-methyl-[1,4]diazepan-1-yl]-acetic acid (DATA) is disclosed in Tornesello et al. (Tornesello, et al., Molecules, 2017, 22: 1282), tetrakis(aminomethyl)methane (TAM) and analogs are disclosed in McAuley 1988 (McAuley, et al., Canadian Journal of Chemistry, 1989, 67: 1657), hexadentate tris(3,4-hydroxypyridinone) (THP) and analogs are disclosed in Ma et al. (Ma, et al., Dalton Trans, 2015, 44: 4884).

The diagnostic and/or therapeutic use of some of the above chelators is described in the prior art. For example, 2-hydrazino nicotinamide (HYNIC) has been widely used in the presence of a coligand for incorporation of ^{99m}Tc and ^{186,188}Re (Schwartz, et al., Bioconjug Chem, 1991, 2: 333; Babich, et al., J Nucl Med, 1993, 34: 1964; Babich, et al., Nucl Med Biol, 1995, 22: 25); DTPA is used in Octreoscan® for complexing ¹¹¹In and several modifications are described in the literature (Li, et al., Nucl Med Biol, 2001, 28: 145; Brechbiel, et al., Bioconjug Chem, 1991, 2: 187); DOTA-type chelators for radiotherapy applications are described by Tweedle et al. (U.S. Pat. No. 4,885,363); other polyaza macrocycles for chelating trivalent isotopes metals are described by Eisenwiener et al. (Eisenwiener, et al., Bioconjug Chem, 2002, 13: 530); and N4-chelators such as a ^{99m}Tc-N4-chelator have been used for peptide labeling in the case of minigastrin for targeting CCK-2 receptors (Nock, et al., J Nucl Med, 2005, 46: 1727).

In an embodiment the chelator is a metal chelator is selected from the group, but not limited to, comprising DOTA, DOTAGA, DOTAM, DOTP, NOTA, NODAGA, NODA-MPAA, HBED, TETA, CB-TE2A, DTPA, CHX-A″-DTPA, DFO, Macropa, HOPO, TRAP, THP, DATA, NOPO, NOTP, PCTA, sarcophagine, FSC, NETA, NE3TA, H4octapa, pycup, HYNIC, NxS4-x (N4, N2S2, N3S), ^{99m}Tc(CO)₃-chelators and their analogs. Preferably, the chelator additionally comprises one or more functional groups or functionalities allowing attachment to the compound of the invention.

The chemical structures thereof being as follows:

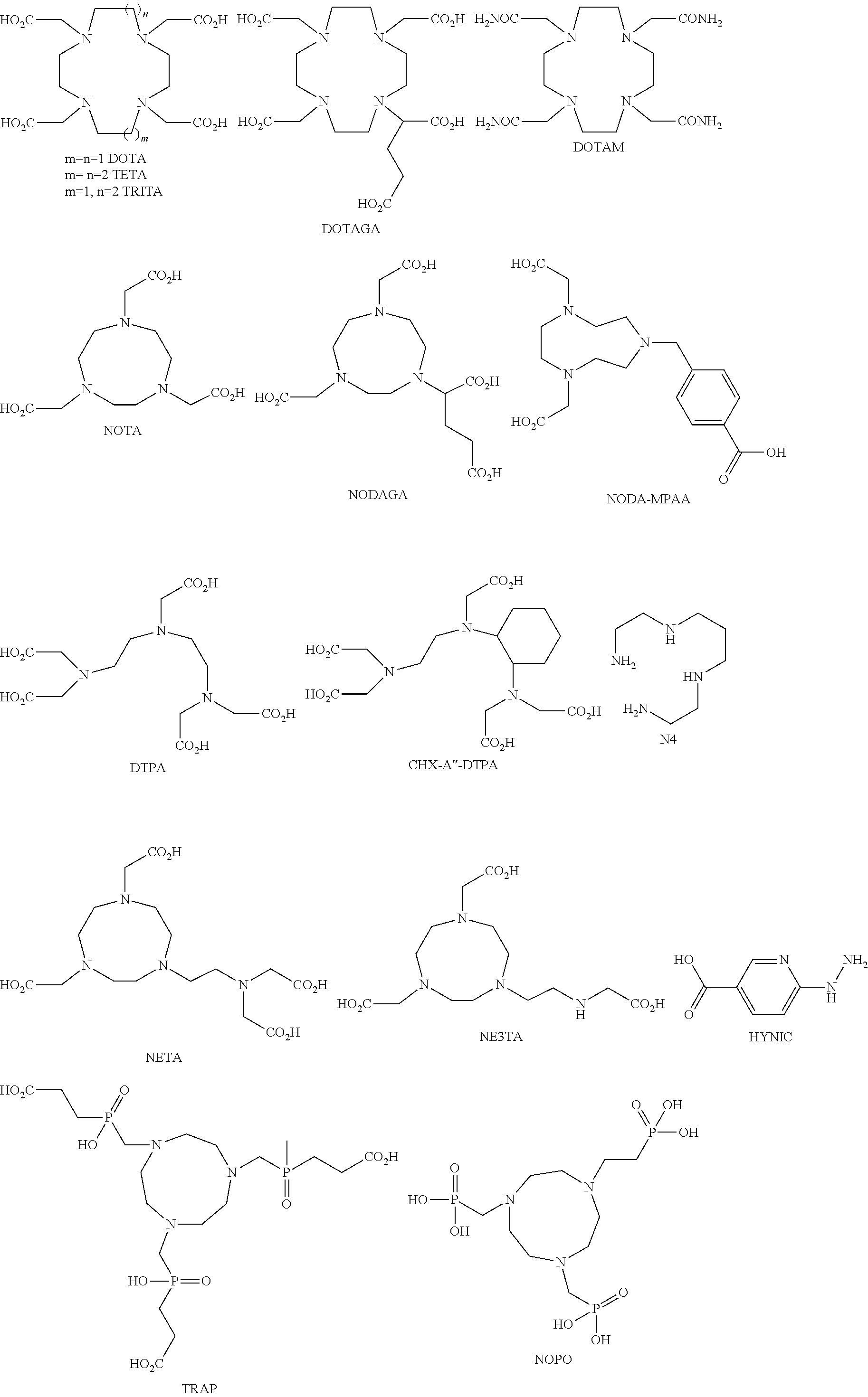

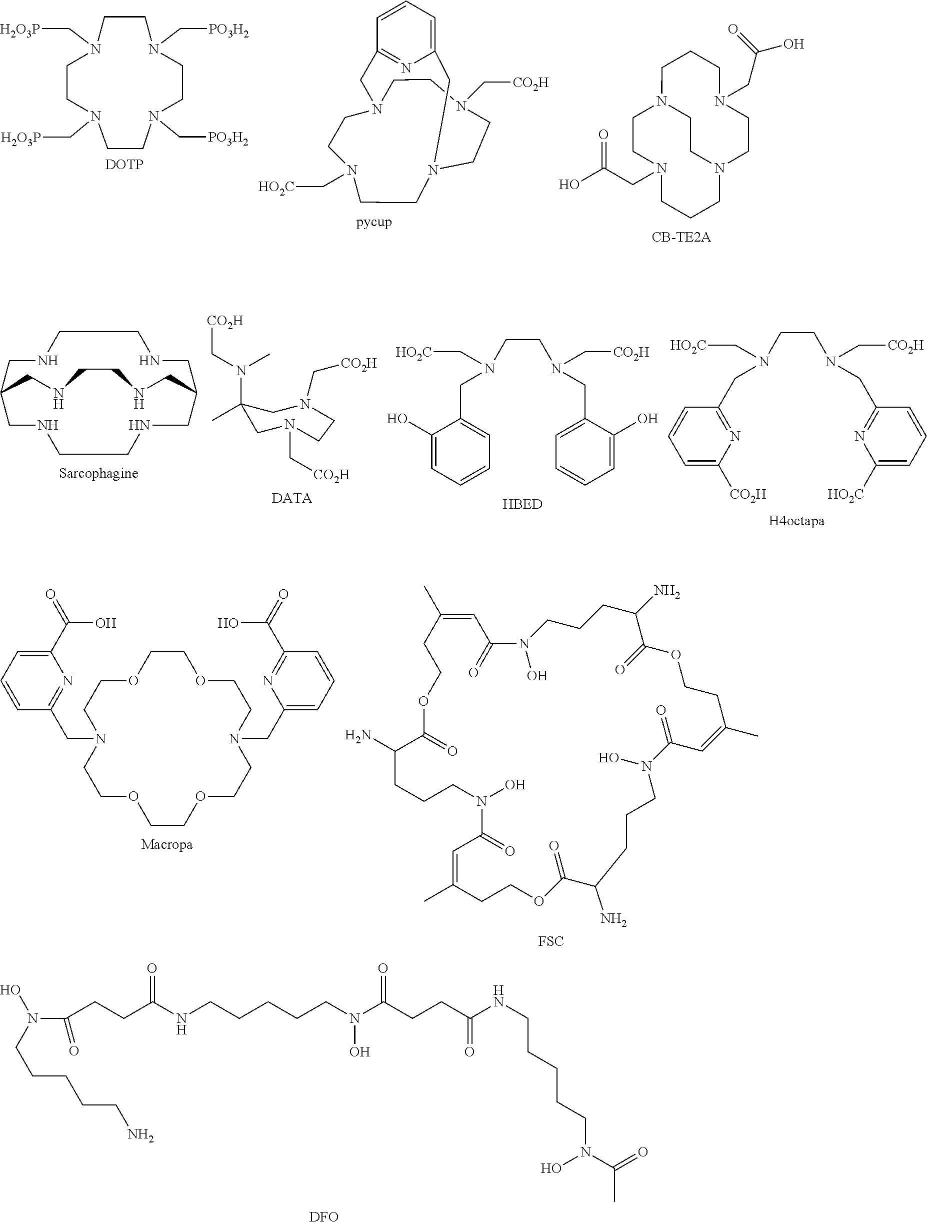

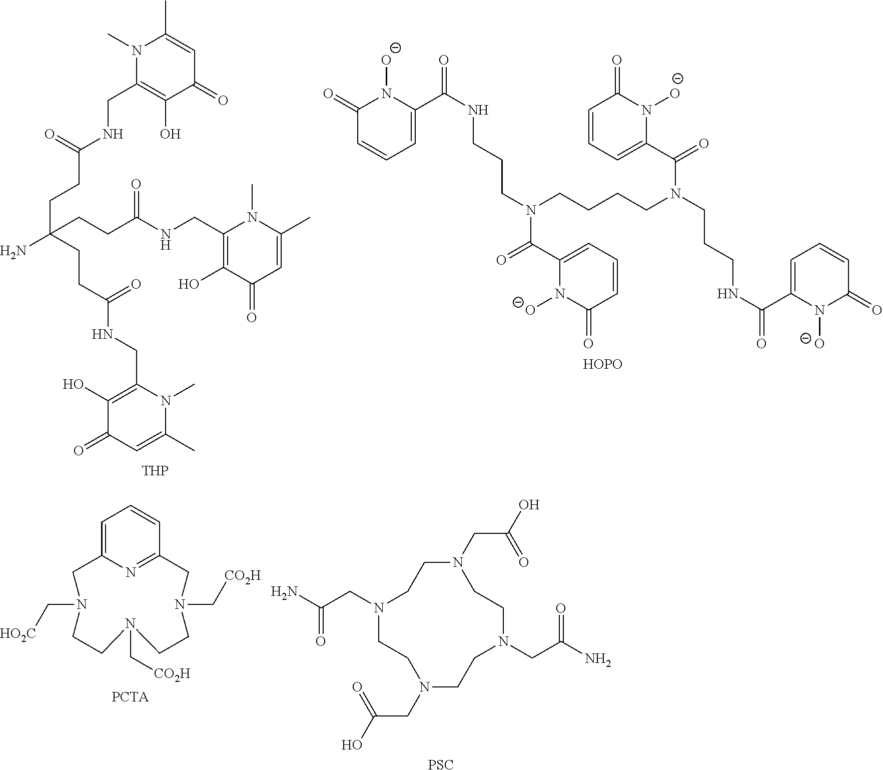

In a preferred embodiment, the metal chelator is selected from the group consisting of DOTA, DOTAGA, DOTAM, NOTA, NODAGA, NODA-MPAA, NOPO, HBED, DTPA, CHX-A″-DTPA, CB-TE2A, Macropa, PCTA, PSC, N4, and analogs thereof. Preferably, the chelator additionally comprises one or more functional groups or functionalities allowing attachment to the compounds of the invention.

In a more preferred embodiment, the metal chelator is selected from the group consisting of DOTA, DOTAGA, NODAGA, and macropa and their analogs thereof. Preferably, the chelator additionally comprises one or more functional groups or functionalities allowing attachment to the compounds of the invention.

It will be acknowledged by the persons skilled in the art that the chelator, in principle, may be used regardless whether the compound of the invention is used in or suitable for diagnosis or therapy. Such principle is, among others, outlined in international patent application WO 2009/109332 A1.

It will be further acknowledged by the persons skilled in the art that the presence of a chelator in the compound of the invention includes, if not stated otherwise, the possibility that the chelator is complexed to any metal complex partner, i.e. any metal which, in principle, can be complexed by the chelator. An explicitly mentioned chelator of a compound of the invention or the general term chelator in connection with the compound of the invention refers either to the uncomplexed chelator as such or to the chelator to which any metal complex partner is bound, wherein the metal complex partner is any radioactive or non-radioactive metal complex partner. Preferably the chelator-metal complex, i.e. the chelator to which the metal complex partner is bound, is a stable chelator-metal complex.

Non-radioactive chelator-metal complexes have several applications, e.g., for assessing properties like stability or activity which are otherwise difficult to determine. One aspect is that cold variants of the radioactive versions of the metal complex partner (e.g., non-radioactive indium complexes es described in the examples) can act as surrogates of the radioactive compounds. Furthermore, they are valuable tools for identifying metabolites in vitro or in vivo, as well as for assessing toxicity properties of the compounds of invention. Additionally, chelator-metal complexes can be used in binding assays utilizing the fluorescence properties of some metal complexes with distinct ligands (e.g., Europium salts).

Chelators can be synthesized or are commercially available with a wide variety of (possibly already activated) groups for the conjugation to peptides or amino acids.

Direct conjugation of a chelator to an amino-nitrogen of the respective compound of invention is well possible for chelators selected from the group consisting of DTPA, DOTA, DOTAGA, NOTA, NODAGA, NODA-MPAA, HBED, TETA, CB-TE2A, DFO, DATA, sarcophagine and N4, preferably DTPA, DOTA, DOTAGA, NOTA, NODAGA, NODA-MPAA, CB-TE2A, and N4. The preferred linkage in this respect is an amide linkage.

Direct conjugation of an isothiocyanate-functionalized chelator to an amino-nitrogen of the respective compound of invention is well possible for chelators selected from the group consisting of DOTA, DOTAGA, NOTA, NODAGA, DTPA, CHX-A″-DTPA, DFO, and THP, preferably DOTA, DOTAGA, NOTA, NODAGA, DTPA, and CHX-A″-DTPA. The preferred linkage in this respect is a thiourea linkage.

Functional groups at a chelator which are preferred precursors for the direct conjugation of a chelator to an amino-nitrogen are known to the person skilled in the art and include but are not limited to carboxylic acid, activated carboxylic acid, e.g., active ester like for instance NHS-ester, pentafluorophenol-ester, HOBt-ester, HOAt-ester, and isothiocyanate.

Functional groups at a chelator which are preferred precursors for the direct conjugation of a chelator to a carboxylic group are known to the person skilled in the art and include but are not limited to alkylamino and arylamino nitrogens. Respective chelator reagents are commercially available for some chelators, e.g., for DOTA with either alkylamino or arylamino nitrogen.

Functional groups at a chelator which are preferred precursors for the direct conjugation of a chelator to a thiol group are known to the person skilled in the art and include but are not limited to maleimide nitrogens. Respective chelator reagents are commercially available for some chelators, e.g., for DOTA with maleimide nitrogen.

Functional groups at a chelator which are preferred precursors for the direct conjugation of a chelator to an azide group are known to the person skilled in the art and include but are not limited to acyclic and cyclic alkynes. Respective chelator reagents are commercially available for some chelators, e.g., for DOTA with propargyl or butynyl.

Functional groups at a chelator which are preferred precursors for the direct conjugation of a chelator to an alkyne group are known to the person skilled in the art and include but are not limited to alkyl and aryl azines. Respective chelator reagents are commercially available for some chelators, e.g., for DOTA with azidopropyl.

In an embodiment, the compound of the invention comprises a bio-distribution modifier. Preferably a bio-distribution modifier is part of the compound of the invention, whereby the bio-distribution modifier is either directly or indirectly such as by a linker attached to the compound of the invention. More preferably a second bio-distribution modifier is part of the compound of the invention, whereby the second bio-distribution modifier is either directly or indirectly such as by a linker attached to the compound of the invention.

The function of a bio-distribution can be the attenuation of the unintended or undesired bio-distribution of the compound of the invention by altering physio-chemical properties of the compound of the invention. To that end elements with a multitude of hydroxyl groups are implemented within or are attached to the peptide of the invention (Wayua, et al., Mol Pharm, 2015, 12: 2477).

The function of a bio-distribution can be the alteration of serum half-life of the compound by modulation of the renal clearance (Du, et al., Bioconjugate Chem, 2020, 31: 241).

The function of a bio-distribution can be the modulation of serum half-life of the compound of the invention by binding to serum components. (Lorenz, et al., Bioorganic Med Chem Lett, 2013, 23: 4011)

In an embodiment an amino-oligo-alcohol, represents the building block for a bio-distribution modifier. The term oligo means that building block may contain 1 to 7 hydroxyl groups. Within the amino-oligo-alcohol the amino function is the conjugation group for the attachment to the peptide of the invention. Usually the amino-oligo-alcohol is linked to the peptide of invention via a dicarboxylic acid linker (such as glutaric acid) or an α-amino acid linker (such as glutamic acid). Preferred amino-oligo-alcohol building blocks comply with formulae (bio01).

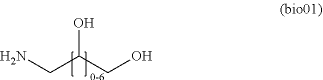

More preferred amino-oligo-alcohol building blocks such as of formula (bio01) are 3-Amino-propane-1,2-diol, 4-Amino-butane-1,2,3-triol, 5-Amino-pentane-1,2,3,4-tetraol, 6-Amino-hexane-1,2,3,4,5-pentaol and 1-amino-1-deoxy-D-glucitol. The most preferred amino-oligo-alcohol building block such as formula (bio01) is 1-amino-1-deoxy-D-glucitol.

In an embodiment, conjugates consisting of two amino-oligo-alcohol building blocks such as of formulae (bio01) or (bio02) attached to the carboxylic acid moieties of an α-amino acid linker such as glutamic acid, represents the building block for a bio-distribution modifier. The resulting construct is connected to the peptide of the invention by dicarboxylic acid linker. Preferably said dicarboxylic acid linker is glutaric acid. A preferred conjugate containing two amino-oligo-alcohol building blocks is a structure of formula (bio02)

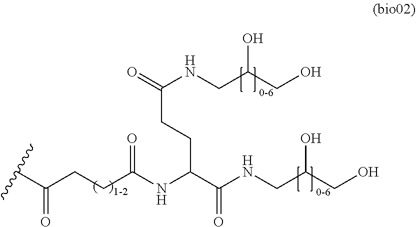

In an embodiment 2-aminopropane-1,3-diol or 2-amino-2-(hydroxymethyl)propane-1,3-diol, represents the building block for a bio-distribution modifier. Within 2-aminopropane-1,3-diol or 2-amino-2-(hydroxymethyl)propane-1,3-diol the respective amino function is the conjugation group for the attachment to the peptide.

In a specific embodiment two units of 2-aminopropane-1,3-diol or 2-amino-2-(hydroxymethyl)propane-1,3-diol are coupled to glutamic acid to result in a preferred conjugate such as formula (bio03) wherein the indicated amino function is the conjugation group for the attachment to the peptide.

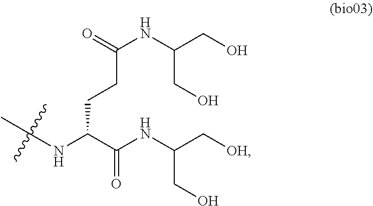 [SMILES: *N[C@H](CCC(=O)NC(CO)CO)C(=O)NC(CO)CO]

It will be appreciated by a person skilled in the art that the 2-aminopropane-1,3-diol units in formula (bio03) can be replaced by 2-amino-2-(hydroxymethyl)propane-1,3-diol. It will further be appreciated by a person skilled in the art that the glutamic acid in formula (bio03) can be replaced by other amino acids with an additional carboxylic acid function.

In a specific embodiment two units of 2-aminopropane-1,3-diol or 2-amino-2-(hydroxymethyl)propane-1,3-diol are coupled to nitrilotriacetic acid to result in a preferred conjugate such as formula (bio04), wherein the indicated carbonyl function is the conjugation group for the attachment to the peptide.

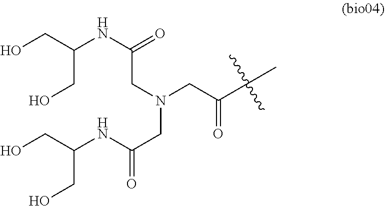 [SMILES: *C(=O)CN(CC(=O)NC(CO)CO)CC(=O)NC(CO)CO]

It will be appreciated by a person skilled in the art that the 2-amino-2-(hydroxymethyl)propane-1,3-diol units in formula (bio03) can be replaced by 2-aminopropane-1,3-diol. It will further be appreciated by a person skilled in the art that the nitrilotriacetic acid in formula (bio04) can be replaced by other compounds with three carboxylic acid functions.

In an embodiment, an inosamine (1-amino-1-deoxy-inositol) represents the building block for a bio-distribution modifier. Within the inosamine the amino function is the conjugation group for the attachment to the peptide of the invention. Usually, the inosamine is linked to the peptide of invention via a dicarboxylic acid linker (such as glutaric acid) or an alpha amino acid linker (such as glutamic acid). Preferred inosamine building blocks comply with formulae (bio05), a more preferred inosamine building block is scyllo-inosamine (bio06).

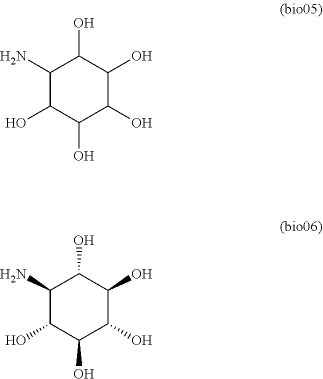 [SMILES: NC1C(O)C(O)C(O)C(O)C1O ; N[C@@H]1[C@@H](O)[C@H](O)[C@@H](O)[C@H](O)[C@H]1O]

In an embodiment, a glyosylated building block represents the building block for a bio-distribution modifier. Preferred glycosyl building blocks comply with formula (bio07).

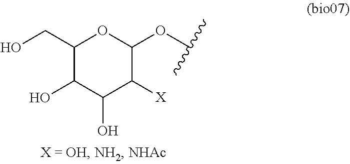

More preferred glycosyl building blocks are glucose, galactose, mannose, glucosamine, galactosamine, mannosamine, N-acetylglucosamine, N-acetylgalactosamine and N-acetylmannosamine.

The sugar moiety is attached to the side chain of an amino acid to from an O- or N-glycosylation as shown in formulae (bio08) and (bio09) to form conjugates which can be employed to attach the glycosyl moiety to the peptide of the invention.

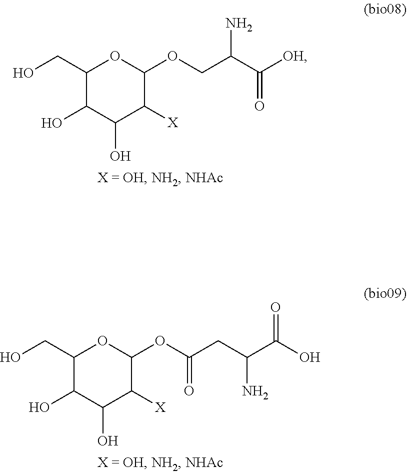

It will be appreciated by a person skilled in the art that the amino acids in formulae (bio08) and (bio09) can be replaced by omega-hydroxy carbonic acids and di-carbonic acids, respectively.

The resulting conjugates can be employed to attach the glycosyl moiety to peptide of the invention likewise.

Furthermore, any suitable form of neo-glycosylation (Advanced Drug Delivery Reviews, Volume 171, April 2021, Pages 62-76) for example such as the attachment of the glycosyl residue via a thioether linkage and or via a triazole linkage may employed to attach a glycosyl moiety to the peptide of the invention.

In an embodiment, a linear monodisperse polyethylene glycol (PEG), represents the building block for a bio-distribution modifier. PEG is derived from ethylene glycol. PEGs with a precisely defined number of ethylene glycol units, which is also termed monodisperse, are preferred as building blocks for PEGylation as bio-distribution modifiers for the present invention. As a non-limiting example formula (bio10) is shown as a building block for a bio-distribution modifier.

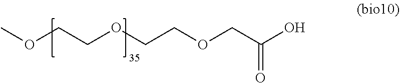

The carboxylic acid moiety in (bio10) is attached covalently to the amino function at the N-terminus of the peptide of invention or to an amino function of an amino acid residue of the peptide of invention. A linker may be interspersed between the monodisperse PEG building block and the peptide of invention.

In an embodiment, a linear polydisperse polyethylene glycol (PEG), represents the building block for a bio-distribution modifier. The synthesis of monodisperse PEGs is rather laborious and hence expensive. Therefore, usually also polydisperse PEGs are employed for the purpose of PEGylation, which means that the polydisperse PEG of a certain molecular weight contains a mixture of different PEGs with a discrete distribution numbers of polyethylene glycol units. For example, a polydiserse PEG with an average molecular weight of 2000 Da consists of a discrete mixture of PEG molecules ranging from about 40 ethylene glycol units to about 55 ethylene glycol units. In the present invention, also polydisperse PEGs can be employed as building blocks for a bio-distribution modifier.

Polydisperse PEG building blocks used as bio-distribution modifier have an average molecular weight of about 2000 Da (40-55 ethylen glycol units), about 5000 Da (95-135 ethylen glycol units), about 10000 Da (195-265 ethylen glycol units) and about 20000 Da (380-540 ethylene glycol units). As non-limiting examples formulae (bio11)-(bio13) are shown as building blocks for a bio-distribution modifier.

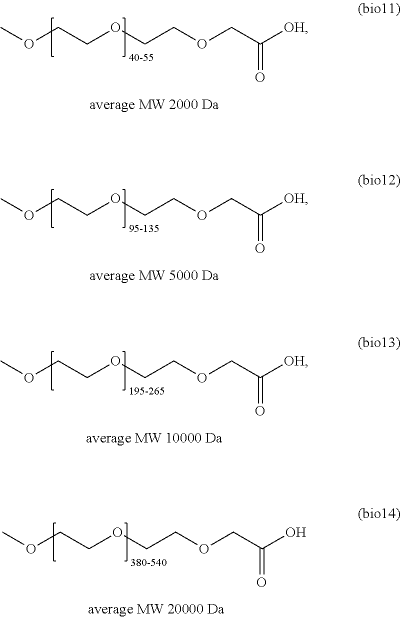

The carboxylic acid moiety in structure (bio10) is attached covalently to the amino function at the N-terminus of the peptide of invention or to an amino function of an amino acid residue of the peptide of invention. A linker may be interspersed between the monodisperse PEG building block and the peptide of the invention.

In an embodiment, a branched polyethylene glycol (PEG), represents the building block for a bio-distribution modifier. The branched PEG may consist of 2 or 3 linear PEG branches/arms/chains. The linear PEG branches/arms/chains implemented in the branched PEG can be monodisperse or polydispers. The individual linear PEG branches/arms/chains implemented in the branched PEG are usually equal. Exemplary branching elements structures are shown in bold in formulae (bio15) to (bio18).

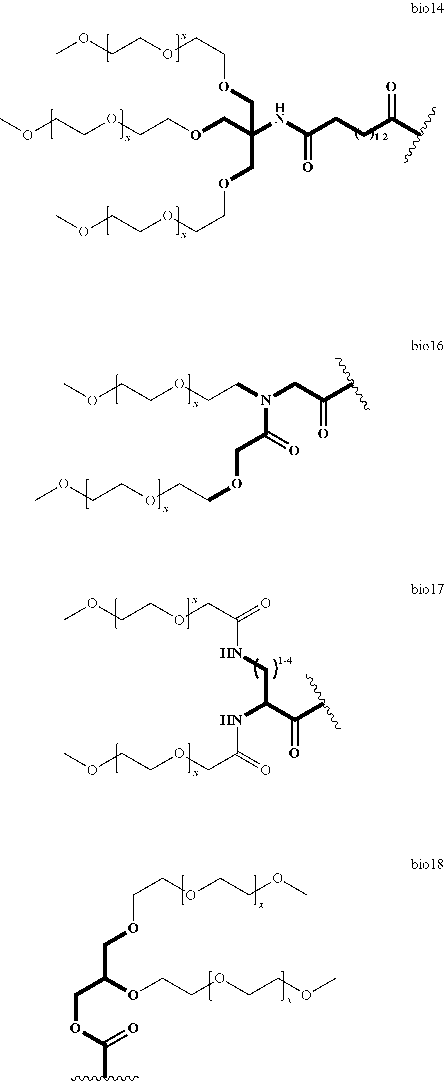

In an embodiment, an carboxylic acid with a long aliphatic chain or an alpha,omega dicarboxylic acid with a long aliphatic chain separating both of the carboxylic acid moieties within said building block, represents a building block for a bio-distribution modifier, which is bound to the peptide of the invention. In case a carboxylic acid is the building block for a bio-distribution modifier, the carboxylic acid moiety is attached covalently to the amino function at the N-terminus of the peptide of invention or to an amino function of an amino acid residue of the peptide of invention. In case a dicarboxylic acid is the building block for a bio-distribution modifier, one of the two carboxylic acid moieties is attached to the amino function at the N-terminus of the peptide of invention or to an amino function of an amino acid residue of the peptide of invention. A linker may be interspersed between either of the carboxylic acid or the dicarboxylic acid and the peptide of invention.

Preferred carboxylic acid building blocks comply with formula (bio19) and preferred carboxylic dicarboxylic acids comply with formula (bio20).

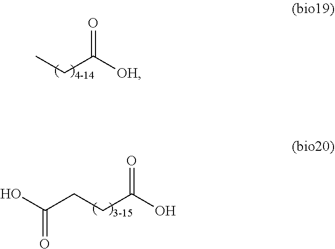

More preferred carboxylic acid building blocks such as formula (bio19) are hexanoic acid, octanoic acid, decanoic acid, lauric acid, myristic acid and palmitic acid. More preferred dicarboxylic acid building blocks such as formula (bio20) are suberic acid, sebacic acid, dodecanedioic acid, tetradecanedioic acid, hexadecanedioic acid and octadecanedioic acid.

In a further embodiment, a glutamic acid is interspersed between either the carboxylic acid or the dicarboxylic acid as shown in formulae (bio21) and (bio22).

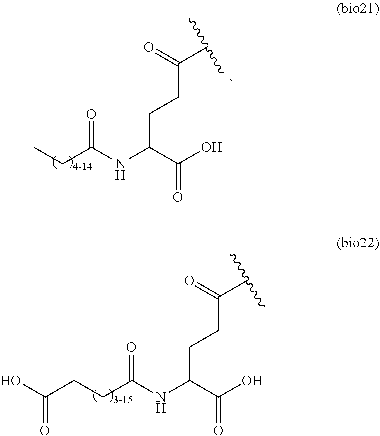

In case a glutamic acid is included in the bio-distribution modifier, a linker may be interspersed between the peptide of invention and the glutamic acid.

In an embodiment, the compound of the invention is present as a pharmaceutically acceptable salt.

A “pharmaceutically acceptable salt” of the compound of the present invention is preferably an acid salt or a base salt that is generally considered in the art to be suitable for use in contact with the tissues of human beings or animals without excessive toxicity or carcinogenicity, and preferably without irritation, allergic response, or other problem or complication. Such salts include mineral and organic acid salts of basic residues such as amines, as well as alkali or organic salts of acidic residues such as carboxylic acids. Compounds of the invention are capable of forming internal salts which are also pharmaceutically acceptable salts.

Suitable pharmaceutically acceptable salts include, but are not limited to, salts of acids such as hydrochloric, phosphoric, hydrobromic, malic, glycolic, fumaric, sulfuric, sulfamic, sulfanilic, formic, toluenesulfonic, methanesulfonic, benzene sulfonic, ethane disulfonic, 2-hydroxyethylsulfonic, nitric, benzoic, 2-acetoxybenzoic, citric, tartaric, lactic, stearic, salicylic, glutamic, ascorbic, pamoic, succinic, fumaric, maleic, propionic, hydroxymaleic, hydroiodic, phenylacetic, alkanoic such as acetic, HOOC—(CH₂)ₙ—COOH where n is any integer from 0 to 4, i.e., 0, 1, 2, 3, or 4, and the like. Similarly, pharmaceutically acceptable cations include, but are not limited to sodium, potassium, calcium, aluminum, lithium and ammonium. Those of ordinary skill in the art will recognize further pharmaceutically acceptable salts for the compounds provided herein. In general, a pharmaceutically acceptable acid or base salt can be synthesized from a parent compound that contains a basic or acidic moiety by any conventional chemical method. Briefly, such salts can be prepared by reacting the free acid or base forms of these compounds with a stoichiometric amount of the appropriate base or acid in water or in an organic solvent, or in a mixture of the two. Generally, the use of non-aqueous media, such as ether, ethyl acetate, ethanol, isopropanol or acetonitrile, is preferred.

A “pharmaceutically acceptable solvate” of the compound of the invention is preferably a solvate of the compound of the invention formed by association of one or more solvent molecules to one or more molecules of a compound of the invention. Preferably, the solvent is one which is generally considered in the art to be suitable for use in contact with the tissues of human beings or animals without excessive toxicity or carcinogenicity, and preferably without irritation, allergic response, or other problem or complication. Such solvent includes an organic solvent such as alcohols, ethers, esters and amines.

A “hydrate” of the compound of the invention is formed by association of one or more water molecules to one or more molecules of a compound of the invention. Such hydrate includes but is not limited to a hemi-hydrate, mono-hydrate, dihydrate, trihydrate and tetrahydrate. Independent of the hydrate composition all hydrates are generally considered as pharmaceutically acceptable.

The compound of the invention has a high binding affinity to the GIPR. The affinity is determined as described in example 27 by the FACS binding assay. High binding affinity is pIC₅₀ category preferably A-D, more preferably A-C, even more preferably A-B and most preferably A. Because of this high binding affinity, the compound of the invention is effective as, useful as and/or suitable as a targeting agent and, if conjugated to another moiety, as a targeting moiety. As preferably used herein a targeting agent is an agent which interacts with the target molecule which is in the instant case said GIPR. In terms of cells and tissues thus targeted by the compound of the invention any cell and tissue, respectively, expressing said GIPR is or may be targeted.

In an embodiment, the compound interacts with a gastric inhibitory polypeptide receptor (GIPR), preferably with human GIPR having an amino acid sequence of SEQ ID NO: 1 or a homolog thereof, wherein the amino acid sequence of the homolog has an identity of GIPR that is at least 85% to the amino acid sequence of SEQ ID NO: 1. In preferred embodiments, the identity is 90%, preferably 95%, 96%, 97%, 98% or 99%.

The identity between two nucleic acid molecules can be determined as known to the person skilled in the art. More specifically, a sequence comparison algorithm may be used for calculating the percent sequence homology for the test sequence(s) relative to the reference sequence, based on the designated program parameters. The test sequence is preferably the sequence or protein or polypeptide which is said to be identical or to be tested whether it is identical, and if so, to what extent, to a different protein or polypeptide, whereby such different protein or polypeptide is also referred to as the reference sequence and is preferably the protein or polypeptide of wild type, more preferably the human GIPR of SEQ ID NO: 1.

Optimal alignment of sequences for comparison can be conducted, e.g., by the local homology algorithm of Smith & Waterman (Smith, et al., Advances in Applied Mathematics, 1981, 2: 482), by the homology alignment algorithm of Needleman & Wunsch (Needleman, et al., J Mol Biol, 1970, 48: 443), by the search for similarity method of Pearson & Lipman (Pearson, et al., Proc Natl Acad Sci USA, 1988, 85: 2444), by computerized implementations of these algorithms (GAP, BESTFIT, FASTA, and TFASTA in the Wisconsin Genetics Software Package, Genetics Computer Group, 575 Science Dr., Madison, Wis.), or by visual inspection.

One example of an algorithm that is suitable for determining percent sequence identity is the algorithm used in the basic local alignment search tool (hereinafter “BLAST”), see, e.g. Altschul et al., 1990 (Altschul, et al., J Mol Biol, 1990, 215: 403) and Altschul et al., 1997 (Altschul, et al., Nucleic Acids Res, 1997, 25: 3389). Software for performing BLAST analyses is publicly available through the National Center for Biotechnology Information (hereinafter “NCBI”). The default parameters used in determining sequence identity using the software available from NCBI, e.g., BLASTN (for nucleotide sequences) and BLASTP (for amino acid sequences) are described in McGinnis et al. (McGinnis, et al., Nucleic Acids Res, 2004, 32: W20).

It is within the present invention that the compound of the invention is used or is for use in a method for the treatment of a disease as disclosed herein. Such method, preferably, comprises the step of administering to a subject in need thereof a therapeutically effective amount of the compound of the invention. Such method includes, but is not limited to, curative or adjuvant cancer treatment. It is used as palliative treatment where cure is not possible and the aim is for local disease control or symptomatic relief or as therapeutic treatment where the therapy has survival benefit and it can be curative.

The method for the treatment of a disease as disclosed herein includes the treatment of the disease disclosed herein, including tumors and cancer, and may be used either as the primary therapy or as second, third, fourth or last line therapy. It is also within the present invention to combine the compound of the invention with further therapeutic approaches. It is well known to the person skilled in the art that the precise treatment intent including curative, adjuvant, neoadjuvant, therapeutic, or palliative treatment intent will depend on the tumor type, location, and stage, as well as the general health of the patient.

In an embodiment, the diseases which may be treated by the compound of the invention is selected from the group comprising medullary thyroid cancer, gastrointestinal neuroendocrine neoplasms, ileal neuroendocrine neoplasms, pancreatic neuroendocrine neoplasms, nonfunctioning neuroendocrine neoplasms, Insulinomas, Gastrinomas, Glucagonomas, VIPomas, Somatostatinoma, ACTHoma, lung neuroendocrine neoplasms, typical carcinoid tumors, atypical carcinoid tumors, small cell carcinoma of the lung, large cell carcinoma of the lung, thymic neuroendocrine tumors, Merkel cell carcinoma, Pheochromocytoma of the adrenal gland, adrenal cancer, parathyroid cancer, paraganglioma, pituitary gland tumors, neuroendocrine tumors of the ovaries and neuroendocrine tumors of the testicles.

In a further embodiment, the disease which may be treated by the compound of the invention is a metabolic disease. Preferably, the metabolic disease is one where GIPR is expressed, more preferably where the GIPR expression is increased in pancreatic beta cells. In a still further embodiment, the disease is selected from the group comprising metabolic diseases and disorders, preferably type 2 diabetes, type 1 diabetes, metabolic syndrome, insulin resistance, dyslipidemia, impaired fasting glucose, and impaired glucose tolerance.

In an embodiment, the compounds of the present invention are used to detect cells and tissues overexpressing the GIPR, whereby such detection is achieved by conjugating a detectable label to the compounds of the invention, preferably a detectable radionuclide. In a preferred embodiment, the cells and tissues detected are diseased cells and tissues and/or are either a or the cause for the disease and/or the symptoms of the disease, or are part of the pathology underlying the disease. In a further preferred embodiment, the diseased cells and tissues are causing and/or are part of an oncology indication (e.g., neoplasms, tumors, and cancers) or a non-oncology indication (e.g., metabolic diseases, preferably type 2 diabetes, insulin restistance, impaired glucose tolerance).

In another embodiment, the compounds of the present invention are used to treat cells and tissues overexpressing the GIPR. In a preferred embodiment, the cells and tissues treated are diseased cells and tissues and/or are either a or the cause for the disease and/or the symptoms of the disease, or are part of the pathology underlying the disease. In a further preferred embodiment, the diseased cells and tissues are causing and/or are part of an oncology indication (e.g. neoplasms, tumors, and cancers) and the therapeutic activity is achieved by conjugating therapeutically active effector to the compounds of the present invention, preferably a therapeutically active radionuclide. In a further preferred embodiment, the diseased cells and tissues are causing and/or are part of a non-oncology indication (e.g., metabolic diseases, insulin resistance, type 1 and type 2 diabetes) and the therapeutic activity is either achieved by targeting of the GIPR or it is monitored by GIPR imaging.

In a further embodiment, particularly if the disease is a non-oncology disease or a non-oncology indication (e.g. metabolic diseases, insulin resistance, type 1 and type 2 diabetes), the compounds of the present invention are administered in therapeutically effective amounts; preferably the compound of the present invention does not comprise a therapeutically active nuclide such as a therapeutically active nuclide An effective amount is a dosage of the compound sufficient to provide a therapeutically or medically desirable result or effect in the subject to which the compound is administered. The effective amount will vary with the particular condition being treated, the age and physical condition of the subject being treated, the severity of the condition, the duration of the treatment, the nature of the concurrent or combination therapy (if any), the specific route of administration and like factors within the knowledge and expertise of the health practitioner. For example, in connection with methods directed towards treating subjects having a condition characterized by abnormal cell proliferation, an effective amount to inhibit proliferation would be an amount sufficient to reduce or halt altogether the abnormal cell proliferation so as to slow or halt the development of or the progression of a cell mass such as, for example, a tumor. As used in the embodiments, “inhibit” embraces all of the foregoing.

In other embodiments, a therapeutically effective amount will be an amount necessary to extend the dormancy of micrometastases or to stabilize any residual primary tumor cells following surgical or drug therapy.

Generally, when using an unconjugated compound without a therapeutically active radionuclide, a therapeutically effective amount will vary with the subject's age, condition, and sex, as well as the nature and extent of the disease in the subject, all of which can be determined by one of ordinary skill in the art. The dosage may be adjusted by the individual physician or veterinarian, particularly in the event of any complication. A therapeutically effective amount is typically, but not limited to, an amount in a range from 0.1 g/kg to about 2000 mg/kg, or from 1.0 μg/kg to about 1000 mg/kg, or from about 0.1 mg/kg to about 500 mg/kg, or from about 1.0 mg/kg to about 100 mg/kg, in one or more dose administrations daily, for one or more days. If desired, the effective daily dose of the active compound may be administered as two, three, four, five, six, or more sub-doses for example administered separately at appropriate intervals throughout the day, optionally, in unit dosage forms. In some embodiments, the compounds are administered for more than 7 days, more than 10 days, more than 14 days and more than 20 days. In still other embodiments, the compound is administered over a period of weeks, or months. In still other embodiments, the compound is delivered on alternate days. For example, the agent is delivered every two days, or every three days, or every four days, or every five days, or every six days, or every week, or every month.

In a preferred embodiment, the compound of the present invention is for use in the treatment and/or prevention of a disease, whereby such treatment is radionuclide therapy.

Preferably, radionuclide therapy makes use of or is based on different forms of radiation emitted by a radionuclide. Such radiation can, for example, be any one of radiation of photons, radiation of electrons including but not limited to β⁻-particles and Auger-electrons, radiation of protons, radiation of neutrons, radiation of positrons, radiation of α-particles or an ion beam. Depending on the kind of particle or radiation emitted by said radionuclide, radionuclide therapy can, for example, be distinguished as photon radionuclide therapy, electron radionuclide therapy, proton radionuclide therapy, neutron radionuclide therapy, positron radionuclide therapy, α-particle radionuclide therapy or ion beam radionuclide therapy. All of these forms of radionuclide therapy are encompassed by the present invention, and all of these forms of radionuclide therapy can be realized by the compound of the invention, preferably under the proviso that the radionuclide attached to the compound of the invention, more preferably as an effector, is providing for this kind of radiation.

Radionuclide therapy preferably works by damaging the DNA of cells. The damage is caused by a photon, electron, proton, neutron, positron, α-particle or ion beam directly or indirectly ionizing the atoms which make up the DNA chain. Indirect ionization happens as a result of the ionization of water, forming free radicals, notably hydroxyl radicals, which then damage the DNA.

In the most common forms of radionuclide therapy, most of the radiation effect is through free radicals. Because cells have mechanisms for repairing DNA damage, breaking the DNA on both strands proves to be the most significant technique in modifying cell characteristics. Because cancer cells generally are undifferentiated and stem cell-like, they reproduce more, and have a diminished ability to repair sub-lethal damage compared to most healthy differentiated cells. The DNA damage is inherited through cell division, accumulating damage to the cancer cells, causing them to die or reproduce more slowly.

Oxygen is a potent radiosensitizer, increasing the effectiveness of a given dose of radiation by forming DNA-damaging free radicals. Therefore, use of high-pressure oxygen tanks, blood substitutes that carry increased oxygen, hypoxic cell radiosensitizers such as misonidazole and metronidazole, and hypoxic cytotoxins, such as tirapazamine may be applied.

Other factors that are considered when selecting a radioactive dose include whether the patient is receiving chemotherapy, whether radiation therapy is being administered before or after surgery, and the degree of success of surgery.

The total radioactive dose may be fractionated, i.e. spread out over time in one or more treatments for several important reasons. Fractionation allows normal cells time to recover, while tumor cells are generally less efficient in repair between fractions. Fractionation also allows tumor cells that were in a relatively radio-resistant phase of the cell cycle during one treatment to cycle into a sensitive phase of the cycle before the next fraction is given. Similarly, tumor cells that were chronically or acutely hypoxic and, therefore, more radioresistant, may reoxygenate between fractions, improving the tumor cell kill.

It is generally known that different cancers respond differently to radiation therapy. The response of a cancer to radiation is described by its radiosensitivity. Highly radiosensitive cancer cells are rapidly killed by modest doses of radiation. These include leukemias, most lymphomas, and germ cell tumors.

It is important to distinguish radiosensitivity of a particular tumor, which to some extent is a laboratory measure, from “curability” of a cancer by an internally delivered radioactive dose in actual clinical practice. For example, leukemias are not generally curable with radiotherapy, because they are disseminated through the body. Lymphoma may be radically curable if it is localized to one area of the body. Similarly, many of the common, moderately radioresponsive tumors can be treated with curative doses of radioactivity if they are at an early stage. This applies, for example, to non-melanoma skin cancer, head and neck cancer, non-small cell lung cancer, cervical cancer, anal cancer, prostate cancer.

The response of a tumor to radiotherapy is also related to its size. For complex reasons, very large tumors respond less well to radiation than smaller tumors or microscopic disease. Various strategies are used to overcome this effect. The most common technique is surgical resection prior to radiotherapy. This is most commonly seen in the treatment of breast cancer with wide local excision or mastectomy followed by adjuvant radiotherapy. Another method is to shrink the tumor with neoadjuvant chemotherapy prior to radical radionuclide therapy. A third technique is to enhance the radiosensitivity of the cancer by giving certain drugs during a course of radiotherapy. Examples of radiosensiting drugs include, but are not limited to Cisplatin, Nimorazole, and Cetuximab.

Introperative radiotherapy is a special type of radiotherapy that is delivered immediately after surgical removal of the cancer. This method has been employed in breast cancer (TARGeted Introperative radioTherapy), brain tumors and rectal cancers.

Radionuclide therapy is in itself painless. Many low-dose palliative treatments cause minimal or no side effects. Treatment to higher doses may cause varying side effects during treatment (acute side effects), in the months or years following treatment (long-term side effects), or after re-treatment (cumulative side effects). The nature, severity, and longevity of side effects depends on the organs that receive the radiation, the treatment itself (type of radionuclide, dose, fractionation, concurrent chemotherapy), and the patient.

It is within the present inventions that the method for the treatment of a disease of the invention may realize each and any of the above strategies which are as such known in the art, and which insofar constitute further embodiments of the invention.

It is also within the present invention that the compound of the invention is used in a method for the diagnosis of a disease as disclosed herein. Such method, preferably, comprises the step of administering to a subject in need thereof a diagnostically effective amount of the compound of the invention.

In accordance with the present invention, an imaging method is selected from the group consisting of scintigraphy, Single Photon Emission Computed Tomography (SPECT) and Positron Emission Tomography (PET).

Scintigraphy is a form of diagnostic test or method used in nuclear medicine, wherein radiopharmaceuticals are internalized by cells, tissues and/or organs, preferably internalized in vivo, and radiation emitted by said internalized radiopharmaceuticals is captured by external detectors (gamma cameras) to form and display two-dimensional images. In contrast thereto, SPECT and PET forms and displays three-dimensional images. Because of this, SPECT and PET are classified as separate techniques to scintigraphy, although they also use gamma cameras to detect internal radiation. Scintigraphy is unlike a diagnostic X-ray where external radiation is passed through the body to form an image.

Single Photon Emission Tomography (SPECT) scans are a type of nuclear imaging technique using gamma rays. They are very similar to conventional nuclear medicine planar imaging using a gamma camera. Before the SPECT scan, the patient is injected with a radiolabeled chemical emitting gamma rays that can be detected by the scanner. A computer collects the information from the gamma camera and translates this into two-dimensional cross-sections. These cross-sections can be added back together to form a three-dimensional image of an organ or a tissue. SPECT involves detection of gamma rays emitted singly, and sequentially, by the radionuclide provided by the radiolabeled chemical. To acquire SPECT images, the gamma camera is rotated around the patient. Projections are acquired at defined points during the rotation, typically every 3-6 degrees. In most cases, a full 360 degree rotation is used to obtain an optimal reconstruction. The time taken to obtain each projection is also variable, but 15-20 seconds is typical. This gives a total scan time of 15-20 minutes. Multi-headed gamma cameras are faster. Since SPECT acquisition is very similar to planar gamma camera imaging, the same radiopharmaceuticals may be used.

Positron Emitting Tomography (PET) is a non-invasive, diagnostic imaging technique for measuring the biochemical status or metabolic activity of cells within the human body. PET is unique since it produces images of the body's basic biochemistry or functions. Traditional diagnostic techniques, such as X-rays, CT scans, or MRI, produce images of the body's anatomy or structure. The premise with these techniques is that any changes in structure or anatomy associated with a disease can be seen. Biochemical processes are also altered by a disease, and may occur before any gross changes in anatomy. PET is an imaging technique that can visualize some of these early biochemical changes. PET scanners rely on radiation emitted from the patient to create the images. Each patient is given a minute amount of a radioactive pharmaceutical that either closely resembles a natural substance used by the body or binds specifically to a receptor or molecular structure. As the radioisotope undergoes positron emission decay (also known as positive beta decay), it emits a positron, the antiparticle counterpart of an electron. After traveling up to a few millimeters, the positron encounters an electron and annihilates, producing a pair of annihilation (gamma) photons moving in opposite directions. These are detected when they reach a scintillation material in the scanning device, creating a burst of light, which is detected by photomultiplier tubes or silicon avalanche photodiodes. The technique depends on simultaneous or coincident detection of the pair of photons. Photons that do not arrive in pairs, i.e., within a few nanoseconds, are ignored. All coincidences are forwarded to the image processing unit where the final image data is produced using image reconstruction procedures.

SPECT/CT and PET/CT is the combination of SPECT and PET with computed tomography (CT). The key benefits of combining these modalities are improving the reader's confidence and accuracy. With traditional PET and SPECT, the limited number of photons emitted from the area of abnormality produces a very low-level background that makes it difficult to anatomically localize the area. Adding CT helps determine the location of the abnormal area from an anatomic perspective and categorize the likelihood that this represents a disease.

It is within the present inventions that the method for the diagnosis of a disease of the invention may realize each and any of the above strategies which are as such known in the art, and which insofar constitute further embodiments of the invention.

Compounds of the present invention are useful to stratify patients, i.e., to create subsets within a patient population that provide more detailed information about how the patient will respond to a given drug. Stratification can be a critical component to transforming a clinical trial from a negative or neutral outcome to one with a positive outcome by identifying the subset of the population most likely to respond to a novel therapy.

Stratification includes the identification of a group of patients with shared “biological” characteristics to select the optimal management for the patients and achieve the best possible outcome in terms of risk assessment, risk prevention and achievement of the optimal treatment outcome.

A compound of the present invention may be used to assess or detect, a specific disease as early as possible (which is a diagnostic use), the risk of developing a disease (which is a susceptibility/risk use), the evolution of a disease including indolent vs. aggressive (which is a prognostic use) and it may be used to predict the response and the toxicity to a given treatment (which is a predictive use).

It is also within the present invention that the compound of the invention is used in a theragnostic method. The concept of theragnostics is to combine a therapeutic agent with a corresponding diagnostic test that can increase the clinical use of the therapeutic drug. The concept of theragnostics is becoming increasingly attractive and is widely considered the key to improving the efficiency of drug treatment by helping doctors identify patients who might profit from a given therapy and hence avoid unnecessary treatments.

The concept of theragnostics is to combine a therapeutic agent with a diagnostic test that allows doctors to identify those patients who will benefit most from a given therapy. In an embodiment and as preferably used herein, a compound of the present invention is used for the diagnosis of a patient, i.e. identification and localization of the primary tumor mass as well as potential local and distant metastases. Furthermore, the tumor volume can be determined, especially utilizing three-dimensional diagnostic modalities such as SPECT or PET. Only those patients having GIPR-positive tumor masses and who, therefore, might profit from a given therapy are selected for a particular therapy and hence unnecessary treatments are avoided. Preferably, such therapy is a GIPR-targeted therapy using a compound of the present invention. In one particular embodiment, chemically identical tumor-targeted diagnostics, preferably imaging diagnostics for scintigraphy, PET or SPECT and radiotherapeutics are applied. Such compounds only differ in the radionuclide and therefore usually have a very similar if not identical pharmacokinetic profile. This can be realized using a chelator and a diagnostic or therapeutic radiometal. Alternatively, this can be realized using a precursor for radiolabeling and radiolabeling with either a diagnostic or a therapeutic radionuclide. In one embodiment diagnostic imaging is used preferably by means of quantification of the radiation of the diagnostic radionuclide and subsequent dosimetry which is known to those skilled in the art and the prediction of drug concentrations in the tumor compared to vulnerable side effect organs. Thus, a truly individualized drug dosing therapy for the patient is achieved.

In an embodiment and as preferably used herein, the theragnostic method is realized with only one theragnostically active compound such as a compound of the present invention labeled with a radionuclide emitting diagnostically detectable radiation (e.g. positrons or gamma rays) as well as therapeutically effective radiation (e.g. electrons or alpha particles).

The invention also contemplates a method of intraoperatively identifying/disclosing diseased tissues expressing GIPR in a subject. Such method uses a compound of the invention, whereby such compound of the invention preferably comprises as Effector a diagnostically active agent.

According to a further embodiment of the invention, the compound of the invention, particularly if complexed with a radionuclide, may be employed as adjunct or adjuvant to any other tumor treatment including, surgery as the primary method of treatment of most isolated solid cancers, radiation therapy involving the use of ionizing radiation in an attempt to either cure or improve the symptoms of cancer using either sealed internal sources in the form of brachytherapy or external sources, chemotherapy such as alkylating agents, antimetabolites, anthracyclines, plant alkaloids, topoisomerase inhibitors, and other antitumor agents, hormone treatments that modulate tumor cell behavior without directly attacking those cells, targeted agents which directly target a molecular abnormality in certain types of cancer including monoclonal antibodies and tyrosine kinase inhibitors, angiogenesis inhibitors, immunotherapy, cancer vaccination, palliative care including actions to reduce the physical, emotional, spiritual, and psycho-social distress to improve the patient's quality of life and alternative treatments including a diverse group of health care systems, practices, and products that are not part of conventional medicine.

In an embodiment of the methods of the invention, the subject is a patient. In an embodiment, a patient is a subject which has been diagnosed as suffering from or which is suspected of suffering from which or which is at risk of suffering from or developing a disease, whereby the disease is a disease as described herein and preferably a disease involving the GIPR.

Dosages employed in practicing the methods for treatment and diagnosis, respectively, where a radionuclide is used and more specifically attached to or part of the compound of the invention will vary depending e.g., on the particular condition to be treated, for example the known radiosensitivity of the tumor type, the volume of the tumor and the therapy desired. In general, the dose is calculated on the basis of radioactivity distribution to each organ and on observed target uptake. A γ-emitting complex may be administered once or at several times for diagnostic imaging. In animals, an indicated dose range may be from 0.1 μg/kg to 5 mg/kg of the compound of the invention complexed e.g., with 1 to 200 MBq of ¹¹¹In or ⁸⁹Zr. A β-emitting complex of the compound of the invention may be administered at several time points e.g., over a period of 1 to 3 weeks or longer. In animals, an indicated dosage range may be of from 0.1 μg/kg to 5 mg/kg of the compound of the invention complexed e.g., with 1 to 200 MBq ⁹⁰Y or ¹⁷⁷Lu. In larger mammals, for example humans, an indicated dosage range is from 0.1 to 100 μg/kg of the compound of the invention complexed with e.g., 10 to 400 MBq ¹¹¹In or ⁸⁹Zr. In larger mammals, for example humans, an indicated dosage range is of from 0.1 to 100 μg/kg of the compound of the invention complexed with e.g., 10 to 5000 MBq ⁹⁰Y or ¹⁷⁷Lu.

In a further aspect, the instant invention is related to a composition and a pharmaceutical composition in particular, comprising the compound of the invention.

The pharmaceutical composition of the present invention comprises at least one compound of the invention and, optionally, one or more carrier substances, excipients and/or adjuvants. The pharmaceutical composition may additionally comprise, for example, one or more of water, buffers such as, e.g., neutral buffered saline or phosphate buffered saline, ethanol, mineral oil, vegetable oil, dimethylsulfoxide, carbohydrates such as e.g., glucose, mannose, sucrose or dextrans, mannitol, proteins, adjuvants, polypeptides or amino acids such as glycine, antioxidants, chelating agents such as EDTA or glutathione and/or preservatives. Furthermore, one or more other active ingredients may, but need not, be included in the pharmaceutical composition of the invention.

The pharmaceutical composition of the invention may be formulated for any appropriate route of administration, including, for example, topical such as, e.g., transdermal or ocular, oral, buccal, nasal, vaginal, rectal or parenteral administration. The term parenteral as used herein includes subcutaneous, intradermal, intravascular such as, e.g., intravenous, intramuscular, intrathecal and intraperitoneal injection, as well as any similar injection or infusion technique. A preferred route of administration is intravenous administration.

In an embodiment of the invention the compound of the invention comprising a radionuclide is administered by any conventional route, in particular intravenously, e.g., in the form of injectable solutions or suspensions. The compound of the invention may also be administered advantageously by infusion, e.g., by an infusion of 30 to 60 min.

Depending on the site of the tumor, the compound of the invention may be administered as close as possible to the tumor site, e.g., by means of a catheter. Such administration may be carried out directly into the tumor tissue or into the surrounding tissue or into the afferent blood vessels. The compound of the invention may also be administered repeatedly in doses, preferably in divided doses.

According to a preferred embodiment of the invention, a pharmaceutical composition of the invention comprises a stabilizer, e.g., a free radical scavenger, which inhibits autoradiolysis of the compound of the invention. Suitable stabilizers include, e.g., serum albumin, ascorbic acid, retinol, gentisic acid or a derivative thereof, or an amino acid infusion solution such, e.g., used for parenteral protein feeding, preferably free from electrolyte and glucose, for example a commercially available amino acid infusion such as Proteinsteril® KE Nephro. Ascorbic acid and gentisic acid are preferred.

A pharmaceutical composition of the invention may comprise further additives, e.g., an agent to adjust the pH between 7.2 and 7.4, e.g., sodium or ammonium acetate or Na₂HPO₄. Preferably, the stabilizer is added to the non-radioactive compound of the invention and introduction of the radionuclide, for instance the complexation with the radionuclide, is performed in the presence of the stabilizer, either at room temperature or, preferably, at a temperature of from 40 to 120° C. The complexation may conveniently be performed under air free conditions, e.g., under N₂ or Ar. Further stabilizer may be added to the composition after complexation.

Excretion of the compound of the invention, particularly if the Effector is a radionuclide, essentially takes place through the kidneys. Further protection of the kidneys from radioactivity accumulation may be achieved by administration of lysine or arginine or an amino acid solution having a high content of lysine and/or arginine, e.g., a commercially available amino acid solution such as Synthamin®-14 or -10, prior to the injection of or together with the compound of the invention, particularly if the Effector is a radionuclide. Protection of the kidneys may also be achieved by administration of plasma expanders such as e.g., gelofusine, either instead of or in addition to amino acid infusion. Protection of the kidneys may also be achieved by administration of diuretics providing a means of forced diuresis which elevates the rate of urination. Such diuretics include high ceiling loop diuretics, thiazides, carbonic anhydrase inhibitors, potassium-sparing diuretics, calcium-sparing diuretics, osmotic diuretics and low ceiling diuretics. A pharmaceutical composition of the invention may contain, apart from a compound of the invention, at least one of these further compounds intended for or suitable for kidney protection, preferably kidney protection of the subject to which the compound of the invention is administered.

It will be understood by a person skilled in the art that the compound of the invention is disclosed herein for use in various methods. It will be further understood by a person skilled in the art that the composition of the invention and the pharmaceutical composition of the invention can be equally used in said various methods. It will also be understood by a person skilled in the art that the composition of the invention and the pharmaceutical composition are disclosed herein for use in various methods. It will be equally understood by a person skilled in the art that the compound of the invention can be equally used in said various methods.

It will be acknowledged by a person skilled in the art that the composition of the invention and the pharmaceutical composition of the invention contain one or more further compounds in addition to the compound of the invention. To the extent that such one or more further compounds are disclosed herein as being part of the composition of the invention and/or of the pharmaceutical composition of the invention, it will be understood that such one or more further compounds can be administered separately from the compound of the invention to the subject which is exposed to or the subject of a method of the invention. Such administration of the one or more further compounds can be performed prior, concurrently with or after the administration of the compound of the invention. It will also be acknowledged by a person skilled in the art that in a method of the invention, apart from a compound of the invention, one or more further compound may be administered to a subject. Such administration of the one or more further compounds can be performed prior, concurrently with or after the administration of the compound of the invention. To the extent that such one or more further compounds are disclosed herein as being administered as part of a method of the invention, it will be understood that such one or more further compounds are part of a composition of the invention and/or of a pharmaceutical composition of the invention. It is within the present invention that the compound of the invention and the one or more further compounds may be contained in the same or a different formulation. It is also within the present invention that the compound of the invention and the one or more further compounds are not contained in the same formulation, but are contained in the same package containing a first formulation comprising a compound of the invention, and a second formulation comprising the one or more further compounds, whereby the type of formulation may be the same or may be different.

It is within the present invention that more than one type of a compound of the invention is contained in the composition of the invention and/or the pharmaceutical composition of the invention. It is also within the present invention that more than one type of a compound of the invention is used, preferably administered, in a method of the invention.

It will be acknowledged that a composition of the invention and a pharmaceutical composition of the invention may be manufactured in conventional manner.

Radiopharmaceuticals have decreasing content of radioactivity with time, as a consequence of the radioactive decay. The physical half-life of the radionuclide is often short for radiopharmaceutical diagnostics. In these cases, the final preparation has to be done shortly before administration to the patient. This is in particular the case for positron emitting radiopharmaceuticals for tomography (PET radiopharmaceuticals). It often leads to the use of semi-manufactured products such as radionuclide generators, radioactive precursors and kits.

Preferably, a kit of the invention comprises apart from one or more than one compounds of the invention typically at least one of the followings: instructions for use, final preparation and/or quality control, one or more optional excipient(s), one or more optional reagents for the labeling procedure, optionally one or more radionuclide(s) with or without shielded containers, and optionally one or more device(s), whereby the device(s) is/are selected from the group comprising a labeling device, a purification device, an analytical device, a handling device, a radioprotection device or an administration device.

Shielded containers known as “pigs” for general handling and transport of radiopharmaceutical containers come in various configurations for holding radiopharmaceutical containers such as bottles, vials, syringes, etc. One form often includes a removable cover that allows access to the held radiopharmaceutical container. When the pig cover is in place, the radiation exposure is acceptable.

A labeling device is selected from the group of open reactors, closed reactors, microfluidic systems, nanoreactors, cartridges, pressure vessels, vials, temperature controllable reactors, mixing or shaking reactors and combinations thereof.

A purification device is preferably selected from the group of ion exchange chromatography columns or devices, size-exclusion chromatography columns or devices, affinity chromatography columns or devices, gas or liquid chromatography columns or devices, solid phase extraction columns or devices, filtering devices, centrifugations vials columns or devices.

An analytical device is preferably selected from the group of tests or test devices to determine the identity, radiochemical purity, radionuclidic purity, content of radioactivity and specific radioactivity of the radiolabelled compound.

A handling device is preferably selected from the group consisting of devices for mixing, diluting, dispensing, labeling, injecting and administering radiopharmaceuticals to a subject.

A radioprotection device is used in order to protect doctors and other personnel from radiation when using therapeutic or diagnostic radionuclides. The radioprotection device is preferably selected from the group consisting of devices with protective barriers of radiation-absorbing material selected from the group consisting of aluminum, plastics, wood, lead, iron, lead glass, water, rubber, plastic, cloth, devices ensuring adequate distances from the radiation sources, devices reducing exposure time to the radionuclide, devices restricting inhalation, ingestion, or other modes of entry of radioactive material into the body and devices providing combinations of these measures.

An administration device is preferably selected from the group of syringes, shielded syringes, needles, pumps, and infusion devices. Syringe shields are commonly hollow cylindrical structures that accommodate the cylindrical body of the syringe and are constructed of lead or tungsten with a lead glass window that allows the handler to view the syringe plunger and liquid volume within the syringe.

It is within the present invention that the term “in an embodiment” or “in an embodiment of the invention” refers to each and any aspect of the invention, including any embodiment thereof.

The present invention is now further illustrated by reference to the following figures and examples from which further features, embodiments and advantages, may be taken, wherein

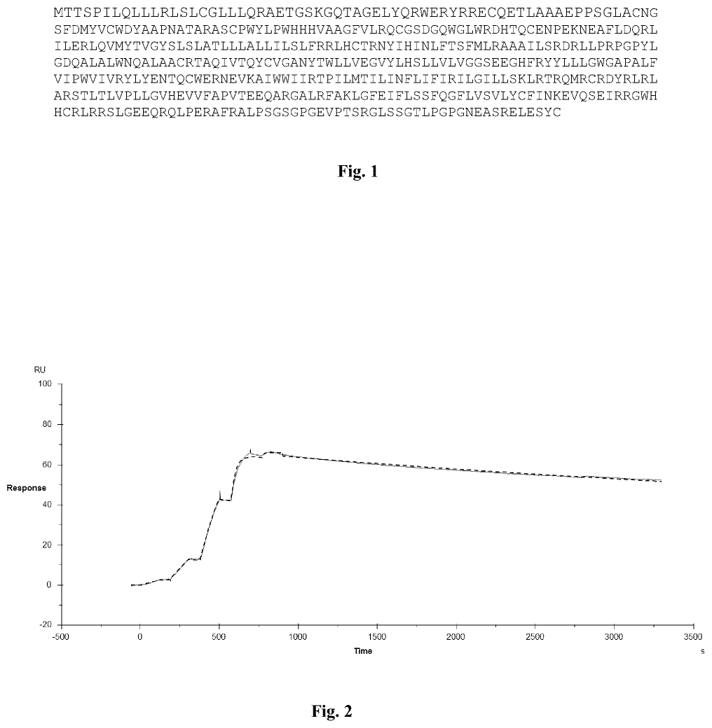

FIG. 1 shows the amino acid sequence of human GIPR (SEQ ID NO: 1);

FIG. 2 shows a representative blank corrected sensorgram for compound GIP-008; blank and reference cell corrected response (RU, Response Unit) data is shown as line; fitted curve shown as dotted black line;

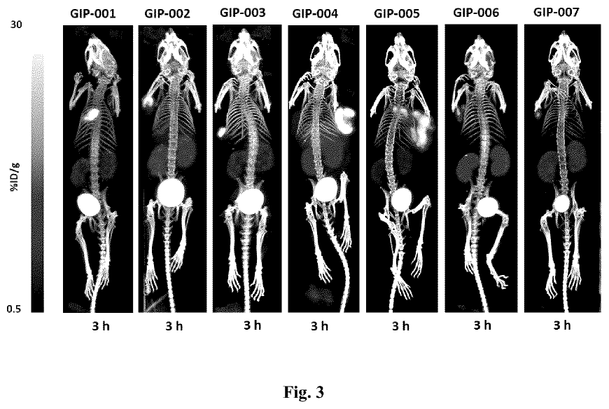

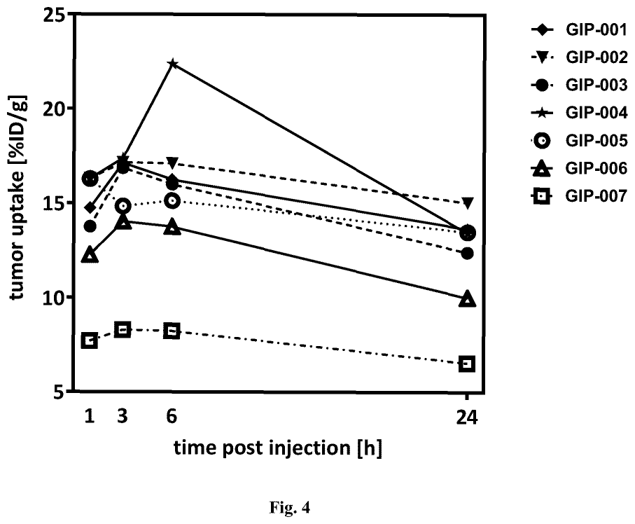

FIG. 3 shows SPECT/CT images of ¹¹¹In-GIP-001, ¹¹¹In-GIP-002, ¹¹¹In-GIP-003, ¹¹¹In-GIP-004, ¹¹¹In-GIP-005, ¹¹¹In-GIP-006, ¹¹¹In-GIP-007 3 h post injection (see, Example 32);

FIG. 4 shows, indicated as % ID/g, quantified tumor uptake as determined by SPECT-imaging of ¹¹¹In-GIP-001, ¹¹¹In-GIP-002, ¹¹¹In-GIP-003, ¹¹¹In-GIP-004, ¹¹¹In-GIP-005, ¹¹¹In-GIP-006, ¹¹¹In-GIP-007 3 h post injection (see, Example 32);

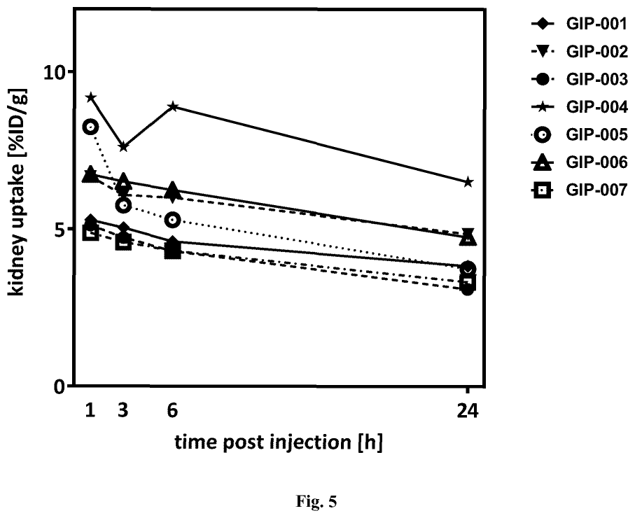

FIG. 5 shows, indicated as % ID/g, quantified kidney uptake as determined by SPECT-imaging of ¹¹¹In-GIP-001, ¹¹¹In-GIP-002, ¹¹¹In-GIP-003, ¹¹¹In-GIP-004, ¹¹¹In-GIP-005, ¹¹¹In-GIP-006, ¹¹¹In-GIP-007 3 h post injection (see, Example 32); and

FIG. 6 shows a scheme illustrating various options for attaching a combination of a chelator optionally comprising a linker and a bio-distribution modifier optionally comprising a linker to a Z group attachment point via an amino acid residue.

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The synthetic descriptions and specific examples that follow are only intended for the purposes of illustration, and are not to be construed as limiting in any manner to make compounds of the disclosure by other methods.

### EXAMPLES

Abbreviations used in the instant application and the following examples in particular are as follows:
° C. means degree celsius
¹¹¹InCl₃ means Indium-111 chloride
¹⁷⁷LuCl₃ means Lutetium-177 chloride
4PL means four parameter logistic
ACN means acetonitrile
ADCC means antibody-dependent cellular cytotoxicity
AGLU means 1-amino-1-deoxy-D-glucitol
Alloc means allyloxycarbonyl
amu means atomic mass unit
aq. means aqueous
AUC means area under the curve
Bio means D-Biotin
BSA means bovine serum albumin
Calc means Calculated
cAMP means cyclic adenosine monophosphate
CAR-T means chimeric antigen receptor T
cDNA means complementary DNA
CHO means chinese hamster ovarian
CM means Chemmatrix™
CMV means cytomegalovirus
CO₂ means carbon dioxide
CT means computed tomography
Da means Dalton
DAD means diode array detector
DCM means dichloromethane
Dde means N-(1-(4,4-dimethyl-2,6-dioxocyclohexylidene)ethyl)
DEG means di(ethylene glycol)
DIC means N,N′-Diisopropylcarbodiimide
DICOM means digital Imaging and Communications in Medicine
DIPEA means N,N-Diisopropylethylamine
DMEM means Dulbecco's Modified Eagle Medium
DMF means dimethylformamide
DMSO means dimethyl sulfoxide
DOTA means dodecane tetraacetic acid-2,2′,2″,2′″-(1,4,7,10-Tetraazacyclododecane-1,4,7,10-tetrayl)tetraacetic acid
DOTA(OtBu)₃-OH means DOTA-tris(tert-butyl ester), Tri-tert-butyl 1,4,7,10-tetraazacyclododecane-1,4,7,10-tetraacetate
DOTA-NHS means DOTA N-hydroxysuccinimide ester
DOTAM means 1,4,7,10-Tetrakis(carbamoylmethyl)-1,4,7,10-tetraazacyclododecane
DOTAM-OH means 1,4,7,10-Tetraazacyclododecane-1,4,7-tri(carbamoylmethyl)-10-acetic acid
DPP4 means dipeptidyl peptidase-4
DTPA means Diethylenetriamine pentaacetate
DTT means Dithiothreitol
e.g. means for example (exempli gratia)
EC₅₀ means half-maximal excitatory concentration
EDT means 1,2-ethanedithiol
EDTA means ethylenediaminetetraacetic acid
ELSD means Evaporative Light Scattering Detector
eq. means equivalent
ESI means electrospray ionization
Et2O means diethylether
EtOAc means ethylacetate
FACS means fluorescence-activated cell sorting
FCS means fetal calf serum
Fmoc means 9-Fluorenyloxycarbonyl
GBq means gigabecquerel
GHRH means Growth hormone-releasing hormone
GIP means glucose-dependent insulinotropic polypeptide
GIPR means glucose-dependent insulinotropic polypeptide receptor
GLP-1 means Glucagon-like peptide-1
GLP-2 means Glucagon-like peptide-2
G-PCR means G-protein coupled receptor
h means hour(s)
H₂O means water
HATU means Hexafluorophosphate Azabenzotriazole Tetramethyl Uronium-1-[Bis(dimethylamino)methylene]-1H-1,2,3-triazolo[4,5-b]pyridinium 3-oxide hexafluorophosphate
HCl means Hydrogen chloride
HEK means Human embryonic kindey
HEPES means 4-(2-hydroxyethyl)-1-piperazineethanesulfonic acid
HFIP means hexafluoro isopropanol
HOAc means acetic acid
HPLC means high performance liquid chromatography
i.e. means that is (id est)
IC50 means half-maximal inhibitory concentration
ID means injected dose
ID/g means injected dose per gram
IDBS means ID Business Solutions
K_{D} means dissociation constant
kDa means 1000 Dalton or Kilo
kDa means kilo Dalton
Kᵢ means inhibitory constant
k_{off} means dissociation rate
kₒₙ means association rate
kVp means peak kilovoltage
LC means Liquid chromatography
LC/TOF-MS means liquid chromatography time-of-flight mass spectrometry
LC-MS means Liquid chromatography mass spectrometry
M means molar or mol per Liter
m/z means mass divided by charge
MAPK means mitogen-activated protein kinase
max. means maximum
MBq means megabecquerel
MeCN means acetonetrile
MeOH means methanol
MeV means mega electron volt
MFI means Median flourescence intensities
min means minute(s)
MTBE means methyl-tert-butyl ether
Mtt means 4-Methyltrityl
MW means molecular weight
n.d. means not determined
N3Tap means L-(4R)-Azidoproline
NaCl means sodium chloride
NaHCO₃ means sodium hydrogencarbonate
NEP means neutral endopeptidase
NET means neuroendocrine tumor
NHS means N-hydroxysuccinimide
NMP means N-Methyl-2-pyrrolidone
NMR means Nuclear Magnetic Resonance
OtBu means tert Butyl ester as protecting group
p.a. means for analytical purpose (quality grade)
PARP means Poly(ADP-ribose) polymerases
PBS means phosphate buffered saline
PBST means phosphate buffered saline containing Tween
pEC₅₀ means the negative log of the EC50 value when converted to molar
PET means positron emission tomography
pH means potential of hydrogen
pIC₅₀ means the negative log of the IC50 value when converted to molar
pK_{D} means negative decadic logarithm of dissociation constant
ppm means parts per million
prep. means preparative
PRRT means peptide receptor radionuclide therapy
PS means Polystyrene
Q-TOF means quadrupole time-of-flight
RFU means relative fluorescence unit
RLU means relative luminescence values
RP means reversed phase
RPM means rounds per minute
RPMI means Roswell Park Memorial Institute
RT means room temperature
Rₜ means retention time
RU means resonance units
SCID means severe combined immunodeficiency
SCK means Single cycle kinetic
sec means second
SPECT means single photon emission computed tomography
SPPS means solid phase peptide synthesis
SPR means surface plasmon resonance
t_{1/2} means Dissociation half-life, equals In 2/k_{off} for 1^{st} order dissociation
process
tBu means tert Butyl ether as protecting group
tBuO means tert Butyl ester as protecting group
TFA means trifluoroacetate or trifluoroacetic acid
TG means tentagel
TIPS means triisopropyl silane
TLC means thin layer chromatography
TOF-MS means tof-of-flight mass spectrometry
UHPLC means ultrahigh performance liquid chromatography
UV means ultraviolet
VGT means vertical gene transfer

### Example 1: Material and Methods

The materials and methods as well as general methods are further illustrated by the following examples.

### Solvents:

Solvents were used in the specified quality without further purification. Acetonitrile (Super Gradient, HPLC, VWR—for analytical purposes; PrepSolv, Merck—for preparative purposes); dichloromethane (synthesis, Roth); ethyl acetate (synthesis grade, Roth); N,N-dimethylformamide (peptide synthesis grade, Biosolve); 1-methyl-2-pyrolidone (peptide grade, IRIS BioTech) 1,4-dioxane (reinst, Roth); methanol (p. a., Merck).

Water: Milli-Q Plus, Millipore, demineralized.

### Chemicals:

Chemicals were synthesized according to or in analogy to literature procedures or purchased from Sigma-Aldrich-Merck (Deisenhofen, Germany), Bachem (Bubendorf, Switzerland), VWR (Darmstadt, Germany), Novabiochem (Merck Group, Darmstadt, Germany), Acros Organics (distribution company Fisher Scientific GmbH, Schwerte, Germany), Iris Biotech (Marktredwitz, Germany), Amatek Chemical (Jiangsu, China), Roth (Karlsruhe, Deutschland), Molecular Devices (Chicago, USA), Biochrom (Berlin, Germany), Peptech (Cambridge, MA, USA), Synthetech (Albany, OR, USA), Pharmacore (High Point, NC, USA), PCAS Biomatrix Inc (Saint-Jean-sur-Richelieu, Quebec, Canada), Alfa Aesar (Karlsruhe, Germany), Tianjin Nankai Hecheng S&T Co., Ltd (Tianjin, China), CheMatech (Dijon, France), JenKem Technology (Plano, TX, USA), Trimen Chemicals (Lodz, Poland) and Anaspec (San Jose, CA, USA) or other companies and used in the assigned quality without further purification.

Boc₄N₄Ac—OH was synthesized according to a literature procedure (Maecke et al. Chem. Eur. J., 2010, 16, 7, 2115).

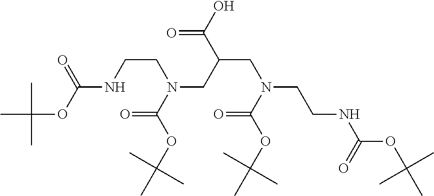 [SMILES: CC(C)(C)OC(=O)NCCN(CC(CN(CCNC(=O)OC(C)(C)C)C(=O)OC(C)(C)C)C(=O)O)C(=O)OC(C)(C)C]

AGLU*—OH was synthesized according to a literature procedure (Vlahov et al. J. Org. Chem., 2010, 75, 3685).

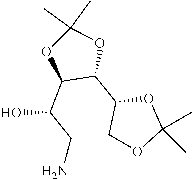 [SMILES: CC1(C)O[C@H]([C@@H](O)CN)[C@@H]([C@H]2COC(C)(C)O2)O1]

β-alanine allyl ester hydrochlorid (H₂N—CH₂CH₂—CO₂Allyl) was synthesized as follows:

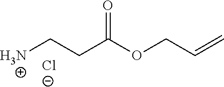

2 g β-alanine (22 mmol) were dissolved in 40 mL of allyl alcohol in a 100 mL three-neck-flask.

The reaction flask was equipped with a capillary dipping into the solution which was further connected with suitable tubing to a two-neck flask filled with sodium chloride (approx. 100 g) via a washing bottle. The sodium chloride filled two-neck flask was equipped with a dropping funnel filled with 30 mL sulphuric acid. The sulphuric acid was then slowly added dropwise to the sodium chloride thereby releasing gaseous HCl which was guided into the stirred solution of β-alanine in allyl alcohol. After the addition of the sulphuric acid was complete the solution of β-alanine was stirred overnight. Then all volatiles were evaporated, the remainder lyophilized from water and acetonitrile to yield 3.5 g (95%) of the target compound which was used without any further purification. ¹H-NMR: (500 MHz, CDCl₃): δ 2.90 (2H, t, J=6.7 Hz), 3.30 (2H, t, J=6.7 Hz), 4.55 (2H, d, J=4.5 Hz), 5.15 (1H, dd, J=9.5, 0.9 Hz), 5.27 (1H, dd, J=16.19, 0.9 Hz), 5.85 (1H, ddt, J=16.19, 9.5, 4.5 Hz).

### HPLC/MS Analyses

HPLC/MS analyses were performed by injection of 5 μL of a solution of the sample, using a 2 step gradient for all chromatograms (5-65% B in 12 min, followed by 65-90% in 0.5 min, A: 0.1% TFA in water and B: 0.1% TFA in ACN). RP columns were from Agilent (Type Poroshell 120, 2.7 μm, EC-C18, 50×3.00 mm, flow 0.8 mL, HPLC at room temperature); Mass spectrometer: Agilent 6230 LC/TOF-MS, ESI ionization. MassHunter Qualitative Analysis B.07.00 SP2 was used as software. UV detection was done at λ=230 nm. Retention times (Rₜ) are indicated in the decimal system (e.g. 1.9 min=1 min 54 s) and are referring to detection in the UV spectrometer. For the evaluation of observed compound masses the ‘Find Compounds by Formula’-feature was used. In particular, the individual ‘neutral mass of a compound (in units of Daltons)’-values and the corresponding isotope distribution pattern were used to confirm compound identity. The accuracy of the mass spectrometer was approx. ±5 ppm.

### HPLC/ELSD Analyse:

HPLC/ELSD analyses of PEGylated peptides with a polydisperse PEG were performed by injection of 25 μL of a solution of the sample, using a 2 step gradient for all chromatograms (20-60% B in 20 min, followed by 60-90% in 5 min, A: 0.1% TFA in water and B: 0.1% TFA in ACN). RP columns were from Vydac (Type PROTEIN & PEPTIDE C18, 5 μm, 150×2.1 mm, flow 0.8 mL, HPLC at 50° C.). UV detection was done at λ=230 nm. For ELSD detection of the PEGylation reagent an Alltech 3300 ELSD device (operating temperature 40° C.) was employed. Retention times (Rₜ) are indicated in the decimal system (e.g. 1.9 min=1 min 54 s) and are referring to detection in the UV spectrometer.

### Preparative HPLC:

Preparative HPLC separations were done with reversed phase columns (General: Kinetex 5μ XB—C18 100 Å, 150×30 mm from Phenomenex; for PEGylated peptides Pursuit 5 C18 200 Å, 150×30 mm from Varian) as stationary phase. As mobile phase 0.1% TFA in water (A) and 0.10% TFA in ACN (B) were used which were mixed in linear binary gradients. The gradients are described as: “10 to 40% B in 30 min”, which means a linear gradient from 10% B (and correspondingly 90% A) to 40% B (and correspondingly 60% A) was run within 30 min. Flow-rates were within the range of 30 to 50 mL/min. A typical gradient for the purification of the compounds of the invention started at 5-25% B and ended after 30 min at 35-50% B and the difference between the percentage B at end and start was at least 10%. A commonly used gradient was “15 to 40% B in 30 min”. Samples were dissolved preferably in mixtures of HOAc and water or DMSO.

### Solid Phase Extraction:

In case of solid phase extraction 250 mg Varian Bondesil-ENV was placed in a 15 mL polystyrene syringe, pre-washed with methanol (1×5 mL) and water (2×5 mL). Then the reaction solution or the solution containing the product to be purified was applied to the column. Thereafter elution was performed with water (2×5 mL—to remove excess salt), 5 mL of 50% ACN in water as first fraction and each of the next fractions were eluted with 5 mL of 50% ACN in water containing 0.1% TFA.

### General Procedures for Automated/Semi-Automated Solid-Phase Synthesis:

Automated solid-phase of peptides and polyamides was performed on a Tetras Peptide Synthesizer (Advanced ChemTech) in 50 μmol and 100 μmol scales. Manual steps were performed in plastic syringes equipped with frits (material PE, Roland Vetter Laborbedarf OHG, Ammerbuch, Germany). The amount of reagents in the protocols described corresponds to the 100 μmol scale, unless stated otherwise.

Solid-phase synthesis was performed on polystyrene (cross linked with 1,4-divinylbenzene (PS) or di (ethylene glycol) dimethacrylate (DEG)), ChemMatrix (CM) or TentaGel (TG) resin. Resin linkers were trityl, wang and rink amide.

### Resin Loading:

In case of the trityl linker the attachment of the first building block (resin loading) was performed as follows. The resin (polystyrene (PS) trityl chloride, initial loading: 1.8 mmol/g) was swollen in DCM (5 mL) for 30 min and subsequently washed with DCM (3 mL, 1 min). Then the resin was treated with a mixture of the corresponding building block (0.5 mmol, 5 eq.) and DIPEA (350 μL, 3.5 mmol, 35 eq.) in DCM (4 mL) for 1 hour. Afterwards the resin was washed with methanol (5 mL, 5 min) and DMF (3 mL, 2×1 min).

In case of the Wang linker pre-loaded resins (polystyrene (PS) and TentaGel (TG)) were employed.

In case of the rink amide linker the attachment of the first residue the resin (CM, DEG) was performed with the same procedure as for the chain assembly as described below.

### Alloc/Allyl-Deprotection:

After swelling in DMF, the resin was washed with DMF and DCM. DCM was de-oxygenated by passing a stream of nitrogen through the stirred solvent. The oxygen-free solvent was used to wash the resin trice. Then 2 mL of a 2 M solution of barbituric acid in oxygen-free DCM and 1 mL of a 25 μM solution of Tetrakis(triphenylphosphine)palladium(0) in oxygen-free DCM were added to the resin. The resin was agitated for 1 hour and then washed with DCM, MeOH, DMF, 0.5% DIPEA in DMF, 0.5% dithiocarbamate in DMF, DMF and DCM (each washing step was repeated 3 times with 3 mL, 1 min).

### Fmoc-Deprotection:

After swelling in DMF, the resin was washed with DMF and then treated with piperidine/DMF (1:4, 3 mL, 2 and 20 min) and subsequently washed with DMF (3 mL, 5×1 min).

### Dde-Deprotection:

After swelling in DMF, the resin was washed with DMF and then treated with hydrazine-hydrate/DMF (2/98, 3 mL 2×10 min) and subsequently washed with DMF (3 mL, 5×1 min).

### Mtt-Deprotection:

After swelling in DCM, the resin was washed with DCM and then treated with HFIP/DCM (7/3, 4-6 mL, 4 hours) and subsequently washed with DCM (3 mL, 3×1 min), DMF (3 mL, 3×1 mL) and DIPEA (0.9 M in DMF, 3 mL, 1 min).

### Solutions of Reagents:

Building Blocks (0.3 M in DMF or NMP), DIPEA (0.9 M in DMF), HATU (0.4 M in DMF), Acetic anhydride (0.75 M in DMF), DIC (3.2 M in DMF)

### Coupling: Coupling of Building Blocks/Amino Acids (Chain Assembly):

Unless otherwise stated, coupling of building blocks was performed as follows: After subsequent addition of solutions of the corresponding building block (0.9 mL, 5 eq.), DIPEA solution (0.6 mL, 10 eq.) and HATU solution (0.65 mL, 5 eq.) the resin was shaken for 45 min. If necessary, the resin was washed with DMF (3 mL, 1 min) and the coupling step was repeated.

In case DOTA(OtBu)₃-OH was coupled as a building block the coupling was performed for 90 min. After this timespan DIC solution was added (0.2 mL, 12.5 eq.) was added and the reaction mixture was agitated for further 90 min.

### Terminal Acetylation:

After addition of DIPEA solution (1.75 mL, 16 eq.) and acetic anhydride solution (1.75 mL, 13 eq.) the resin was shaken for 10 min. Afterwards the resin was washed with DMF (3 mL, 6×1 min).

Cleavage Method A: Cleavage of Protected Fragments from Hyper-Acid Labile Resin:

After the completion of the assembly of the sequence the resin was finally washed with DCM (3 mL, 4×1 min) and then dried in the vacuum. Then the resin was treated with HFIP/DCM (7/1, 4 mL, 4 hours) and the collected solution evaporated to dryness. The residue was purified with preparative HPLC or used without further purification.

### Cleavage Method B: Cleavage of Unprotected Fragments (Complete Resin Cleavage):

After the completion of the assembly of the sequence the resin was finally washed with DCM (3 mL, 4×1 min), dried in the vacuum overnight and treated with TFA, EDT, water and TIPS (94/2.5/2.5/1) for 4 h (unless otherwise stated). Afterwards the cleavage solution was poured into a chilled mixture of MTBE and cyclohexane (1/1, 10-fold excess compared to the volume of cleavage solution), centrifuged at 4° C. for 5 min and the precipitate collected and dried in the vacuum. The residue was lyophilized from water/acetonitrile prior to purification or further modification.

### Cleavage Method C: Cleavage of Protective Groups of Peptides in Solution

The protected/partially protected compound was dissolved in TFA, water and TIPS (95/2.5/2.5) for 2 h (unless otherwise stated). Afterwards the cleavage solution was poured into a chilled mixture of MTBE and cyclohexane (1/1, 10-fold excess compared to the volume of cleavage solution), centrifuged at 4° C. for 5 min and the precipitate collected and dried in the vacuum. The residue was lyophilized from water/acetonitrile prior to purification or further modification.

Cleavate Method D: Cleavage of Unprotected Fragments with Reagent K (Complete Resin Cleavage):

After the completion of the assembly of the sequence the resin was finally washed with DCM (3 mL, 4×1 min), dried in the vacuum overnight and treated with TFA, phenol, water, thioanisole and EDT (82.5/5/5/5/2.5) for 4 h (unless otherwise stated). Afterwards the cleavage solution was poured into a chilled mixture of MTBE and cyclohexane (1/1, 10-fold excess compared to the volume of cleavage solution), centrifuged at 4° C. for 5 min and the precipitate collected and dried in the vacuum. The residue was lyophilized from water/acetonitrile prior to purification or further modification.

More relevant Fmoc-solid-phase-peptide synthesis methods are described in detail in “Fmoc Solid Phase Peptide Synthesis” Editors W. Chan, P. White, Oxford University Press, USA, 2000. Compounds were named using MestreNova version 12 Mnova IUPAC Name plugin (Mestrelab Research, S.L.), or AutoNom version 2.2 (Beilstein Informationssysteme Copyright© 1988-1998, Beilstein Institut fir Literatur der Organischen Chemie licensed to Beilstein Chemiedaten and Software GmbH, where appropriate.

### Solution Cyclization Method A: Dibromoxylene or Bis-(Bromomethyl)-Pyridin Cyclization

The crude peptide material was dissolved in a 1:1 mixture of ammonium bicarbonate solution (50 mM, pH=8.5) and acetonitrile. To the resulting mixture a solution of α,α′-dibromo-m-xylene or 2,6-bis(bromomethyl)pyridine in acetonitrile was added. Upon completion of the cyclization reaction which was judged by analytical LC-MS, DTT was added and the solution stirred for a further hour. Then TFA was added and the reaction solution subjected to lyophilization. The volume of solvent, amount of α,α′-dibromo-m-xylene or 2,6-bis(bromomethyl)pyridine, DTT and volume of TFA used in the reaction depended on the amount of resin used for the synthesis of the linear peptide precursor—per 50 μmol of initially used 60 mL of the solvent mixture, 14.5 mg (55 μmol) of α,α′-dibromo-m-xylene or 14.5 mg (55 μmol), 15.4 mg (100 μmol) of DTT and 50 μL of TFA were used.

Solution Cyclization Method B: Cyclization with Maleimide Derivatives

The crude peptide material was dissolved in a 1:1 mixture of ammonium bicarbonate (0.1 M, pH=8) and acetonitrile. To the resulting mixture a solution of the corresponding 3,4-dibromomaleimide in acetonitrile was added. Upon completion of the cyclization reaction which was judged by LC-MS, TFA was added and the reaction solution subjected to lyophilization. The volume of solvent, amount of 3,4-dibromomaleimide and volume of TFA used in the reaction depended on the amount of resin used for the synthesis of the linear peptide precursor—per 50 μmol of initially used resin 60 mL of the solvent mixture, 14.0 mg (55 μmol) of 3,4-dibromomaleimide and 50 μL of TFA were used.

### Solution Cyclization Method C: Disulfide Cyclization

The crude peptide material was dissolved in a 1:1 mixture of acetonitrile and ammonium acetate buffer (0.1M, pH 6). To the solution [Pt(en)₂Cl₂]Cl₂ (Dichlorobis-(ethylendiamine)-platinum(IV) chloride) was added. Upon completion of the cyclization reaction which was judged by analytical LC-MS, TFA was added and the reaction solution subjected to lyophilization. The volume of solvent, amount of Pt-reagent and volume of TFA used in the reaction depended on the amount of resin used for the synthesis of the linear peptide precursor—per 50 μmol of initially used 60 mL of the solvent mixture, 22.8 mg (50 μmol) of Pt-reagent and 50 μL of TFA were used.

### Preparation of Compounds:

Specific embodiments for the preparation of compounds of the invention are prepared by using the preferred general methods disclosed above and other published or other methods which are known to persons skilled in the art Unless otherwise specified, all starting materials and reagents are of standard commercial grade, and are used without further purification, or are readily prepared from such materials by routine methods. Those skilled in the art of organic synthesis will recognize in light of the instant disclosure that starting materials and reaction conditions may be varied including additional steps employed to produce compounds encompassed by the present invention. Since one important step is the cyclization towards different cyclic structures these general methods are disclosed below in a separate section.

### Example 2: Synthesis of H-Met-Tyr-Asp-Trp-Trp-Pro-Glu-Leu-[Cys(3MeBn)-Ala-Leu-Ile-Cys]-Ttds-Lys(Bio)-NH₂ (GIP-057)

The linear sequence (H-Met-Tyr-Asp-Trp-Trp-Pro-Glu-Leu-[Cys(3MeBn)-Ala-Leu-Ile-Cys]-Ttds-Lys(Bio)-NH₂) of the peptide was assembled according to the ‘General procedures for Automated/Semi-automated Solid-Phase Synthesis’ in a 100 μmol scale on a Rink amide resin. After performing the steps of ‘Cleavage method B’ (2 hours) the lyophilized crude peptide residue was subjected to ‘Solution Cyclization method A’ with dibromoxylene as cyclization reagent. After lyophilization the crude product was purified by preparative HPLC (35 to 65% B in 30 min—Kinetex) to yield 9.38 mg of the pure title compound (15.0%). HPLC t_{R}=9.24 min. LC/TOF-MS: exact mass 2399.129 (calculated 2399.123). C₁₁₅H₁₆₆N₂₂O₂₆S₄ (MW=2400.949).

### Example 3: Synthesis of DOTA-Ttds-Tyr-Asp-Trp-Trp-Pro-Glu-Leu-[Cys(3MeBn)-Ala-Leu-Ile-Cys]-NH₂ (GIP-126)

The linear sequence (DOTA-Ttds-Tyr-Asp-Trp-Trp-Pro-Glu-Leu-Cys-Ala-Leu-Ile-Cys-NH₂) of the peptide was assembled according to the ‘General procedures for Automated/Semi-automated Solid-Phase Synthesis’ in a 50 μmol scale on a Rink amide resin. After performing the steps of ‘Cleavage method B’ the lyophilized crude peptide residue was subjected to ‘Solution Cyclization method A’ with dibromoxylene as cyclization reagent. After lyophilization the crude product was purified by preparative HPLC (35 to 60% B in 30 min—Kinetex) to yield 16.11 mg of the pure title compound (14.0%). HPLC t_{R}=9.29 min. LC/TOF-MS: exact mass 2300.097 (calculated 2300.090). C₁₁₀H₁₅₇N₂₁O₂₉S₂ (MW=2301.683).

### Example 4: Synthesis of Ac-Tyr-Asp-Trp-Trp-Pro-Glu-Leu-[Cys(3MeBn)-Ala-Leu-Ile-Cys]-Ttds-Lys(DOTA)-NH₂ (GIP-127)

The linear sequence (Ac-Tyr-Asp-Trp-Trp-Pro-Glu-Leu-Cys-Ala-Leu-Ile-Cys-Ttds-Lys(Mtt)-NH₂) of the peptide was assembled according to the ‘General procedures for Automated/Semi-automated Solid-Phase Synthesis’ in a 50 μmol scale on a Rink amide resin. The Mtt-group was removed by performing the ‘Mtt-deprotection’ as described in the ‘General procedures for Automated/Semi-automated Solid-Phase Synthesis’, DOTA(OtBu)₃-OH was coupled and the peptide cleaved from the resin by performing the steps of ‘Cleavage method B’ (4 hours cleavage time). The lyophilized crude peptide residue was subjected to ‘Solution Cyclization method A’ with dibromoxylene as cyclization reagent. After lyophilization the crude product was purified by preparative HPLC (35 to 60% B in 30 min—Kinetex) to yield 10.37 mg of the pure title compound (8.4%). HPLC t_{R}=9.37 min. LC/TOF-MS: exact mass 2470.202 (calculated 2470.195). C₁₁₈H₁₇₁N₂₃O₃₁S₂ (MW=2471.893).

### Example 5: Synthesis of DOTA-Ttds-Asp-Trp-Trp-Pro-Glu-leu-[Cys(3MeBn)-Aib-Leu-Ile-AET] (GIP-129)

The linear sequence (DOTA-Ttds-Asp-Trp-Trp-Pro-Glu-leu-Cys-Aib-Leu-Ile-AET-H) of the peptide was assembled according to the ‘General procedures for Automated/Semi-automated Solid-Phase Synthesis’ in a 50 μmol scale on a Cysteamine-4-methoxytrityl resin. After performing the steps of ‘Cleavage method B’ the lyophilized crude peptide residue was subjected to ‘Solution Cyclization method A’ with dibromoxylene as cyclization reagent. After lyophilization the crude product was purified by preparative HPLC (30 to 60% B in 30 min—Kinetex) to yield 43.69 mg of the pure title compound (41.4%). HPLC t_{R}=8.83 min. LC/TOF-MS: exact mass 2108.044 (calculated 2108.036). C₁₀₁H₁₄₉N₁₉O₂₆S₂ (MW=2109.512).

### Example 6: Synthesis of 4OHPhp-Asp-Trp-Trp-Pro-Glu-leu-[Cys(3MeBn)-Aib-Leu-Ile-Cys]-Ttds-Lys(DOTA)-NH₂ (GIP-137)

The linear sequence (4OHPhp-Asp-Trp-Trp-Pro-Glu-leu-Cys-Aib-Leu-Ile-Cys-Ttds-Lys(Mtt)-NH₂) of the peptide was assembled according to the ‘General procedures for Automated/Semi-automated Solid-Phase Synthesis’ in a 50 μmol scale on a Rink amide resin. After the coupling of 3-(4-Hydroxyphenyl) propionic acid, the Mtt-group was removed by performing the ‘Mtt-deprotection’ as described in the ‘General procedures for Automated/Semi-automated Solid-Phase Synthesis’, DOTA(OtBu)₃-OH was coupled and the peptide cleaved from the resin by performing the steps of ‘Cleavage method B’ (4 hours cleavage time). The lyophilized crude peptide residue was subjected to ‘Solution Cyclization method A’ with dibromoxylene as cyclization reagent. After lyophilization the crude product was purified by preparative HPLC (35 to 65% B in 30 min—Kinetex) to yield 14.21 mg of the pure title compound (11.7%). HPLC t_{R}=9.04 min. LC/TOF-MS: exact mass 2427.198 (calculated 2427.190). C₁₁₇H₁₇₀N₂₂O₃₀S₂ (MW=2428.868).

### Example 7: Synthesis of HO-Succinyl-Trp-1Ni-Pro-Glu-Leu-[Cys(3MeBn)-Aib-Leu-Ile-Cys]-Ttds-Lys(DOTA)-NH₂ (GIP-085)

The linear sequence (tBuO-Succinyl-Trp-1Ni-Pro-Glu-Leu-Cys-Aib-Leu-Ile-Cys-Ttds-Lys(Mtt)-NH₂) of the peptide was assembled according to the ‘General procedures for Automated/Semi-automated Solid-Phase Synthesis’ in a 50 μmol scale on a Rink amide resin. After the coupling of mono-tert.-butyl succinate, the Mtt-group was removed by performing the ‘Mtt-deprotection’ as described in the ‘General procedures for Automated/Semi-automated Solid-Phase Synthesis’, DOTA(OtBu)₃-OH was coupled and the peptide cleaved from the resin by performing the steps of ‘Cleavage method B’. The lyophilized crude peptide residue was subjected to ‘Solution Cyclization method A’ with dibromoxylene as cyclization reagent. After lyophilization the crude product was purified by preparative HPLC (35 to 65% B in 30 min—Kinetex) to yield 15.17 mg of the pure title compound (13.30%). HPLC t_{R}=10.40 min. LC/TOF-MS: exact mass 2275.133 (calculated 2275.131). C₁₁₀H₁₆₂N₂₀O₂₈S₂ (MW=2276.717).

### Example 8: Synthesis of DOTA-APAc-Asp-Trp-5Clw-Hyp-Glu-leu-[Dap(2Lut)-Thp-Leu-Ile-AET] (GIP-106)

The linear sequence (DOTA-Ttds-Asp-Trp-Trp-Pro-Glu-leu-Dap-Aib-Leu-Ile-AET-H) of the peptide was assembled according to the ‘General procedures for Automated/Semi-automated Solid-Phase Synthesis’ in a 100 μmol scale on a Cysteamine-4-methoxytrityl resin. After performing the steps of ‘Cleavage method B’ the lyophilized crude peptide residue was subjected to ‘Solution Cyclization method A’ with bis-(bromomethyl)-pyridin as cyclization reagent. After lyophilization the crude product was purified by preparative HPLC (20 to 45% B in 30 min—Kinetex) to yield 10.58 mg of the pure title compound (5.2%). HPLC t_{R}=7.57 min. LC/TOF-MS: exact mass 2021.894 (calculated 2021.948). C₉₅H₁₃₆ClN₂₁O₂₄S (MW=2023.745).

### Example 9: Synthesis of HO-Succinyl-Trp-5Clw-Hyp-Glu-leu-[Cys(2Lut)-Apc(DOTA)-Leu-Ile-AET] (GIP-110)

The linear sequence (tBuO-Succinyl-Trp-5Clw-Hyp-Glu-leu-Cys-Apc-Leu-Ile-AET-H) of the peptide was assembled according to the ‘General procedures for Automated/Semi-automated Solid-Phase Synthesis’ in a 100 μmol scale on a Cysteamine-4-methoxytrityl resin. After performing the steps of ‘Cleavage method B’ the lyophilized crude peptide residue was subjected to ‘Solution Cyclization method A’ with bis-(bromomethyl)-pyridin as cyclization reagent. Prior to the coupling of DOTA the cyclic intermediate peptide (HO-Succinyl-Trp-5Clw-Hyp-Glu-leu-[Cys(2Lut)-Apc-Leu-Ile-AET]) was purified by purified by preparative HPLC (25 to 55% B in 30 min—Kinetex) to yield 83.96 mg of the peptide precursor (56.0%). 40 mg of the intermediate peptide (26.7 μmol) were dissolved in DMSO (400 μL) and the pH value of the solution adjusted to 8 by addition of DIPEA (8 μL). DOTA-NHS (32.3 mg, 40 μmol, 1.5 eq.) was added and the pH value re-adjusted to 8 by addition of further DIPEA. After completion of the reaction the solution was subjected to preparative HPLC (25 to 50% B in 30 min—Kinetex) to yield 28.8 mg of the pure title compound (57.2%—yield for coupling of DOTA—overall yield: 32.0%). HPLC t_{R}=8.68 min. LC/TOF-MS: exact mass 1882.827 (calculated 1882.819). C₈₈H₁₂₃ClN₁₈O₂₂S₂ (MW=1884.614).

### Example 10: Synthesis of AGLU-Glutar-Asp-Trp-5Clw-Hyp-Glu-leu-[Cys(2Lut)-Thp-Leu-Ile-Cys]-APAc-glu(AGLU)-Lys(DOTA)-NH₂ (GIP-004)

The linear sequence (HO-Glutar-Asp-Trp-5Clw-Hyp-Glu-leu-Cys-Thp-Leu-Ile-Cys-APAc-glu(OAll)-Lys(Dde)-NH₂) of the peptide was assembled according to the ‘General procedures for Automated/Semi-automated Solid-Phase Synthesis’ in a 50 μmol scale on a Rink amide resin. The N-terminal Glutaric acid was introduced by reaction of glutaric acid anhydride (5 eq., incl. DIPEA 10 eq.) with peptide resin. The side chain of the C-terminal lysine was liberated from the Dde-group by executing the ‘Dde-deprotection’ followed by the coupling of DOTA(OtBu)₃-OH to the previously liberated amino function. In contrast to the general procedure in the present case during the coupling of the DOTA building block no DIC was added. Afterwards, the allyl ester protecting group was removed acid by performing the steps of the ‘Alloc/Allyl-deprotection’ from D-glutamic acid. AGLU* was coupled simultaneously to the side chain of the latter and the free N-terminal carboxylic acid function of the peptide resin (AGLU* (195 mg, 7.5 eq.), Oxyma (107 mg, 7.5 eq.) and DIC (116 μL, 7.5 eq.), DMF, overnight at 50° C.). Thereafter the steps of ‘Cleavage method B’ where performed to cleave the peptide from the resin. The lyophilized crude peptide residue was subjected to ‘Solution Cyclization method A’ with bis-(bromomethyl)-pyridin as cyclization reagent. After lyophilization the crude product was purified by preparative HPLC (25 to 40% B in 30 min—Kinetex) to yield 10.29 mg of the pure title compound (7.4%). HPLC t_{R}=7.02 min. LC/TOF-MS: exact mass 2779.260 (calculated 2779.253). C₁₂₄H₁₈₇ClN₂₆O₄₀S₂ (MW=2781.552).

### Example 11: Synthesis of HO-Succinyl-Trp-5Clw-Hyp-Glu-leu-[Cys(2Lut)-Apc(AGLU-Glutar)-Leu-Ile-Cys]-APAc-Lys(DOTA)-NH₂ (GIP-073)

The linear sequence (HO-Glutar-Asp-Trp-5Clw-Hyp-Glu-leu-Cys-Apc(Alloc)-Leu-Ile-Cys-APAc-Lys(Mtt)-NH₂) of the peptide was assembled according to the ‘General procedures for Automated/Semi-automated Solid-Phase Synthesis’ in a 50 μmol scale on a Rink amide resin. After the coupling of mono-tert.-butyl succinate, the Mtt-protecting group was removed by performing the ‘Mtt-deprotection’ as described in the ‘General procedures for Automated/Semi-automated Solid-Phase Synthesis’, DOTA(OtBu)₃-OH was coupled and the Alloc protecting group removed from the side chain of the Apc building block. Glutaric acid was coupled to Apc side chain by reaction of the peptide resin with glutaric acid anhydride (31 mg, 5 eq., DIPEA (48 μL, 5 eq.), DMF, 45 min, room temperature). AGLU* was coupled to the carboxylic acid function of the glutaric acid (AGLU* (195 mg, 7.5 eq.), Oxyma (107 mg, 7.5 eq.) and DIC (116 μL, 7.5 eq.), DMF, overnight at 50° C.). Thereafter the steps of ‘Cleavage method B’ where performed to cleave the peptide from the resin. The lyophilized crude peptide residue was subjected to ‘Solution Cyclization method A’ with bis-(bromomethyl)-pyridin as cyclization reagent. After lyophilization the crude product was purified by preparative HPLC (25 to 50% B in 30 min—Kinetex) to yield 14.15 mg of the pure title compound (11.4%). HPLC t_{R}=7.78 min. LC/TOF-MS: exact mass 2471.139 (calculated 2471.131). C₁₁₃H₁₆₇ClN₂₄O₃₂S₂ (MW=2473.266).

### Example 12: Synthesis of AGLU-Glutar-Asp-Trp-5Clw-Hyp-Glu-leu-[Cys(2Lut)-Apc(DOTA)-Leu-Ile-Cys]-APAc-glu(AGLU)-NH₂ (GIP-002)

The linear sequence (Fmoc-Asp-Trp-5Clw-Hyp-Glu-leu-Cys-Apc-Leu-Ile-Cys-APAc-glu(OAll)-NH₂) of the peptide was assembled according to the ‘General procedures for Automated/Semi-automated Solid-Phase Synthesis’ in a 100 μmol scale on a Rink amide resin. The allyl ester protecting group was removed acid by performing the steps of the ‘Alloc/Allyl-deprotection’ from D-glutamic acid. The N-terminal Fmoc-group was removed and glutaric acid was coupled to N-terminus by reaction of the peptide resin with glutaric acid anhydride (57 mg, 5 eq., DIPEA (171 μL, 10 eq.), DMF, 150 min, room temperature). AGLU* was coupled simultaneously to the side chain of the latter and the free N-terminal carboxylic acid function of the peptide resin (AGLU* (195 mg, 7.5 eq.), Oxyma (107 mg, 7.5 eq.) and DIC (116 μL, 7.5 eq.), DMF, overnight at 50° C.). Thereafter the steps of ‘Cleavage method B’ where performed to cleave the peptide from the resin. The lyophilized crude peptide residue was subjected to ‘Solution Cyclization method A’ with bis-(bromomethyl)-pyridin as cyclization reagent. Prior to the coupling of DOTA the cyclic intermediate peptide (AGLU-Glutar-Asp-Trp-5Clw-Hyp-Glu-leu-[Cys(2Lut)-Apc-Leu-Ile-Cys]-APAc-glu(AGLU)-NH₂) was purified by purified by preparative HPLC (20 to 50% B in 30 min—Kinetex) to yield 31.04 mg of the peptide precursor (13.7%). The purified peptide was dissolved in DMSO (300 μL) and the pH value of the solution adjusted to 8 by addition of DIPEA (20 μL). DOTA-NHS (15.6 mg, 20.5 μmol, 1.5 eq.) was added and the pH value re-adjusted to 8 by addition of further DIPEA. After completion of the reaction the solution was subjected to preparative HPLC (25 to 50% B in 30 min—Kinetex) to yield 21.4 mg of the pure title compound (58.9%—yield for coupling of DOTA—overall yield: 8.1%). HPLC t_{R}=7.08 min. LC/TOF-MS: exact mass 2650.178 (calculated 2650.174). C₁₁₈H₁₇₆ClN₂₅O₃₈S₂ (MW=2652.394).

### Example 13: Synthesis of AGLU-Glutar-Asp-Trp-5Clw-Tap-Glu-leu-[Cys(2Lut)-Apc(DOTA)-Leu-Ile-Cys]-APAc-glu(AGLU)-NH₂ (GIP-001)

Synthesis with Coupling of DOTA-NHS and Reduction of Azide Function in Solution

The linear sequence (Fmoc-Asp-Trp-5Clw-N₃Tap-Glu-leu-Cys-Apc-Leu-Ile-Cys-APAc-glu(OAll)-NH₂) of the peptide was assembled according to the ‘General procedures for Automated/Semi-automated Solid-Phase Synthesis’ in a 100 μmol scale on a Rink amide resin. The allyl ester protecting group was removed acid by performing the steps of the ‘Alloc/Allyl-deprotection’ from D-glutamic acid. The N-terminal Fmoc-group was removed and glutaric acid was coupled to N-terminus by reaction of the peptide resin with glutaric acid anhydride (57 mg, 5 eq., DIPEA (171 μL, 10 eq.), DMF, 150 min, room temperature). AGLU* was coupled simultaneously to the side chain of the latter and the free N-terminal carboxylic acid function of the peptide resin (AGLU* (195 mg, 7.5 eq.), Oxyma (107 mg, 7.5 eq.) and DIC (116 μL, 7.5 eq.), DMF, overnight at 50° C.). Thereafter the steps of ‘Cleavage method B’ where performed to cleave the peptide from the resin. The lyophilized crude peptide residue was subjected to ‘Solution Cyclization method A’ with bis-(bromomethyl)-pyridin as cyclization reagent. Prior to the coupling of DOTA the cyclic intermediate peptide (AGLU-Glutar-Asp-Trp-5Clw-N3Tap-Glu-leu-[Cys(2Lut)-Apc-Leu-Ile-Cys]-APAc-glu(AGLU)-NH₂) was purified by purified by preparative HPLC (20 to 50% B in 30 min—Kinetex) to yield 30.94 mg of the peptide precursor (13.5%). The purified peptide was dissolved in DMSO (300 μL) and the pH value of the solution adjusted to 8 by addition of DIPEA (20 μL). DOTA-NHS (15.4 mg, 20.3 μmol, 1.5 eq.) was added and the pH value re-adjusted to 8 by addition of further DIPEA. After completion of the DOTA a solution of triscarboxylethylenephosphane HCl (53 mg in 200 μL DMSO) was added and the solution stirred for 80 min. After completion of the reduction of the azide to the amine the solution was subjected to preparative HPLC (15 to 45% B in 30 min—Kinetex) to yield 17.72 mg of the pure title compound (49.5%—yield for coupling of DOTA—overall yield: 6.7%). HPLC t_{R}=6.57 min. LC/TOF-MS: exact mass 2649.198 (calculated 2649.190). C₁₁₈H₁₇₇ClN₂₆O₃₇S₂ (MW=2651.410).

Synthesis with AGLU*-Glutar-OH and Fmoc-Glu(AGLU*)—OH Building Blocks

The linear sequence (AGLU*-Glutar-Asp-Trp-5Clw-Tap-Glu-leu-Cys-Apc(Alloc)-Leu-Ile-Cys-APAc-glu(AGLU*)—NH₂) of the peptide was assembled according to the ‘General procedures for Automated/Semi-automated Solid-Phase Synthesis’ in a 100 μmol scale on a Rink amide resin. The alloc protection group was removed from the Apc side chain by performing the steps of the ‘Alloc/Allyl-deprotection’. DOTA(OtBu)₃-OH was coupled and the peptide cleaved from the resin Thereafter the steps of ‘Cleavage method D’ where performed to cleave the peptide from the resin. The lyophilized crude peptide residue was subjected to ‘Solution Cyclization method A’ with bis-(bromomethyl)-pyridin as cyclization reagent. After lyophilization the crude product was purified by preparative HPLC (15 to 40% B in 30 min—Kinetex) to yield 43.62 mg of the pure title compound (16.5%). HPLC t_{R}=6.57 min. LC/TOF-MS: exact mass 2649.198 (calculated 2649.190). C₁₁₈H₁₇₇ClN₂₆O₃₇S₂ (MW=2651.410).

Synthesis with AGLU*-Glutar-OH and Fmoc-Glu(AGLU*)—OH Building Blocks and Alternative Cyclization Conditions

The linear sequence (AGLU*-Glutar-Asp-Trp-5Clw-Tap-Glu-leu-Cys-Apc(Alloc)-Leu-Ile-Cys-APAc-glu(AGLU*)—NH₂) of the peptide was assembled according to the ‘General procedures for Automated/Semi-automated Solid-Phase Synthesis’ in a 50 μmol scale on a Rink amide resin. The alloc protection group was removed from the Apc side chain by performing the steps of the ‘Alloc/Allyl-deprotection’. DOTA(OtBu)₃-OH was coupled and the peptide cleaved from the resin Thereafter the steps of ‘Cleavage method D’ where performed to cleave the peptide from the resin. The lyophilized crude peptide residue was subjected to ‘Solution Cyclization method A’ with bis-(bromomethyl)-pyridin as cyclization reagent with the exception that as a solvent for the cyclization reaction a mixture of ethanol and acetonitrile (60 mL, 1:1, adjusted to pH 8 by addition of DIPEA). After evaporation of the volatiles the crude product was purified by preparative HPLC (15 to 40% B in 30 min—Kinetex) to yield 6.8 mg of the pure title compound (5.1%). HPLC t_{R}=6.57 min. LC/TOF-MS: exact mass 2649.198 (calculated 2649.190). C₁₁₈H₁₇₇ClN₂₆O₃₇S₂ (MW=2651.410).

Synthesis with AGLU*-Glutar-OH and Fmoc-Glu(AGLU*)—OH Building Blocks and on Resin Cyclization

The linear sequence (AGLU*-Glutar-Asp-Trp-5Clw-Tap-Glu-leu-Cys(SDmp)-Apc(Alloc)-Leu-Ile-Cys(SDmp)-APAc-glu(AGLU*)—NH₂) of the peptide was assembled according to the ‘General procedures for Automated/Semi-automated Solid-Phase Synthesis’ in a 50 μmol scale on a Rink amide resin. The SDmp-disulfide protecting groups were removed from the cysteine side chains by treating the peptide resin with a 5% DTT solution (500 mg DTT in 10 mL 0.1 N-methylmorpholine in DMF, 3×10 min). The cyclization was achieved by adding bis-(bromomethyl)-pyridin (13.25 mg, 1 eq., DIPEA (43 μL, 5 eq.), DMF, overnight, room temperature) to the resin. The alloc protection group was removed from the Apc side chain by performing the steps of the ‘Alloc/Allyl-deprotection’. DOTA(OtBu)₃-OH was coupled and the peptide cleaved from the resin employing the steps of ‘Cleavage method D’. After lyophilization the crude product was purified by preparative HPLC (15 to 40% B in 30 min—Kinetex) to yield 1.36 mg of the pure title compound (1.0%). HPLC t_{R}=6.57 min. LC/TOF-MS: exact mass 2649.198 (calculated 2649.190). C₁₁₈H₁₇₇ClN₂₆O₃₇S₂ (MW=2651.410).

### Example 14: Synthesis of AGLU-Glutar-Asp-Trp-5Clw-Tap-Glu-leu-[Cys(2Lut)-Apc(NODAGA-Gly)-Leu-Ile-Cys]-APAc-glu(AGLU)-NH₂ (GIP-031)

The linear sequence (AGLU*-Glutar-Asp-Trp-5Clw-Tap-Glu-leu-Cys-Apc(Alloc)-Leu-Ile-Cys-APAc-glu(AGLU*)—NH₂) of the peptide was assembled according to the ‘General procedures for Automated/Semi-automated Solid-Phase Synthesis’ in a 50 μmol scale on a Rink amide resin. The alloc protection group was removed from the Apc side chain by performing the steps of the ‘Alloc/Allyl-deprotection’. Fmoc-Gly-OH and then (R)-NODAGA(tBu)₃-OH was coupled and the peptide cleaved from the resin Thereafter the steps of ‘Cleavage method D’ where performed to cleave the peptide from the resin. The lyophilized crude peptide residue was subjected to ‘Solution Cyclization method A’ with bis-(bromomethyl)-pyridin as cyclization reagent. After lyophilization the crude product was purified by preparative HPLC (15 to 45% B in 30 min—Kinetex) to yield 11.5 mg of the pure title compound (8.6%). HPLC t_{R}=7.10 min. LC/TOF-MS: exact mass 2677.281 (calculated 2677.185). C₁₁₉H₁₇₇ClN₂₆O₃₈S₂ (MW=2679.420).

### Example 15: Synthesis of AGLU-Glutar-APAc-Asp-Trp-5Clw-Hyp-Glu-leu-[Cys(2Lut)-Apc(DOTA)-Leu-Ile-Cys]-lys(MeO36PEG)-NH₂ (GIP-039)

Initially the C-terminal part (Alloc-Apc(Fmoc)-Leu-Ile-Cys]-lys(Dde)-NH₂) of the linear sequence of the peptide was assembled according to the ‘General procedures for Automated/Semi-automated Solid-Phase Synthesis’ in a 50 μmol scale on a Rink amide resin. The Fmoc-group was removed, DOTA(OtBu)₃-OH was coupled and the alloc protection group cleaved from the N-terminus of the peptide resin by performing the steps of the ‘Alloc/Allyl-deprotection’. Thereafter the N-terminal part of the peptide sequence was assembled until the glutaric acid moiety which was introduced by the reaction of the peptide resin with glutaric acid anhydride (31 mg, 5 eq., 0.6 mL 0.9M DIPEA in DMF, 150 min, room temperature). AGLU* was coupled to the free N-terminal carboxylic acid function of the peptide resin (AGLU* (98 mg, 7.5 eq.), Oxyma (54 mg, 7.5 eq.) and DIC (58 μL, 7.5 eq.), DMF, overnight at 50° C.). The side chain of the C-terminal lysine was liberated from the Dde-group by executing the ‘Dde-deprotection’ followed by the coupling the monodisperse MeO36PEG-OH PEG-building block (169 mg, 2 eq., Oyxma (21.5 mg, 3 eq.), DIC (23.4 μL, 3 eq.)). The resin was agitated at 50° C. for 2.5 hours, the addition of DIC repeated and the resin agitated overnight. Thereafter the steps of ‘Cleavage method B’ where performed to cleave the peptide from the resin. The lyophilized crude peptide residue was subjected to ‘Solution Cyclization method A’ with bis-(bromomethyl)-pyridin as cyclization reagent. After lyophilization the crude product was purified by preparative HPLC (25 to 50% B in 30 min—Kinetex) to yield 11.86 mg of the pure title compound (5.7%). HPLC t_{R}=7.66 min. LC/TOF-MS: exact mass 4157.127 (calculated 4157.122). C189H₃₁₈ClN₂₅O₇₀S₂ (MW=4160.265).

### Example 16: Synthesis of Ac-Asp-Trp-5Clw-Hyp-Glu-leu-[Cys(2Lut)-Thp-Leu-Ile-Cys]-OH (GIP-181)

The linear sequence (AGLU*-Glutar-Asp-Trp-5Clw-Tap-Glu-leu-Cys-Apc(Alloc)-Leu-Ile-Cys-APAc-glu(AGLU*)—NH₂) of the peptide was assembled according to the ‘General procedures for Automated/Semi-automated Solid-Phase Synthesis’ in a 50 μmol scale on a Trityl resin. Thereafter the steps of ‘Cleavage method B’ where performed to cleave the peptide from the resin. The lyophilized crude peptide residue was subjected to ‘Solution Cyclization method A’ with bis-(bromomethyl)-pyridin as cyclization reagent. After lyophilization the crude product was purified by preparative HPLC (30 to 60% B in 30 min—Kinetex) to yield 34.09 mg of the pure title compound (42.6%). HPLC t_{R}=8.83 min. LC/TOF-MS: exact mass 1598.643 (calculated 1598.634). C₇₅H₉₉ClN₁₄O₁₉S₂ (MW=1600.259).

### Example 17: Synthesis of AGLU-Glutar-APAc-Asp-Trp-5Clw-Tap(AGLU-Glutar)-Glu-leu-[Cys(2Lut)-Apc(DOTA-glu(AGLU))-Leu-Ile-Cys]-APAc-glu(AGLU)-NH₂ (GIP-007)

Initially the C-terminal part (Dde-Apc(Fmoc)-Leu-Ile-Cys-APAc-glu(OAll)-NH₂) of the linear sequence of the peptide was assembled according to the ‘General procedures for Automated/Semi-automated Solid-Phase Synthesis’ in a 100 μmol scale on a Rink amide resin. The Fmoc-group was removed, Fmoc-glu(OAll)-OH and then DOTA(OtBu)₃-OH were coupled. The N-terminus was released from the protection group by executing the ‘Dde-deprotection’ to yield the linear intermediate H-Apc(DOTA-glu(OAll))-Leu-Ile-Cys]-APAc-glu(OAll)-NH₂ peptide resin. Afterwards the linear precursor peptide was assembled until the N-terminal APAc moiety to yield Fmoc-APAc-Asp-Trp-5Clw-Tap(Alloc)-Glu-leu-Cys-Apc(DOTA-glu(OAll))-Leu-Ile-Cys-APAc-glu(OAll)-NH₂. The alloc and allyl ester protection groups were release simultaneously, the Fmoc group was removed and the N-terminal glutaric acid coupled by the reaction of the peptide resin with glutaric acid anhydride (57 mg, 5 eq., 1.15 mL 0.9M DIPEA in DMF, 150 min, room temperature) to yield HO-Glutar-APAc-Asp(OtBu)-Trp-5Clw-Tap(HO-Glutar)-Glu(OtBu)-leu-Cys-Apc(DOTA-glu)-Leu-Ile-Cys-APAc-glu-NH₂. AGLU* was coupled to the four free carboxylic acid functions by treating the peptide resin with the mixture of AGLU* (392 mg, 15 eq.), Oxyma (214 mg, 15 eq.) and DIC (232 μL, 15 eq.) (in 1.7 mL DMF) for 1.5 hours at 50° C. The addition of DIC was repeated and the resin left to agitate overnight at 50° C. Thereafter the steps of ‘Cleavage method B’ where performed to cleave the peptide from the resin. The lyophilized crude peptide residue was subjected to ‘Solution Cyclization method A’ with bis-(bromomethyl)-pyridin as cyclization reagent. After lyophilization the crude product was purified by preparative HPLC (25 to 50% B in 30 min—Kinetex) to yield 4.16 mg of the pure title compound (1.2%). HPLC t_{R}=6.20 min. LC/TOF-MS: exact mass 3358.542 (calculated 3358.528). C₁₄₇H₂₂₈ClN₃₁O₅₂S₂ (MW=3361.151).

### Example 18: Synthesis of Ac-Asp-Trp-5Clw-Nglu(AGLU)-Glu-leu-[Cys(2Lut)-Thp-Leu-Ile-Cys]-NH₂ (GIP-176)

Initially the C-terminal part (BrAc-Glu-leu-Cys-Thp-Leu-Ile-Cys-NH₂) of the linear sequence of the peptide was assembled according to the ‘General procedures for Automated/Semi-automated Solid-Phase Synthesis’ in a 100 μmol scale on a Rink amide resin. The peptide resin was reacted with β-alanine allyl ester hydrochloride (H₂N—CH₂CH₂—CO₂Allyl-HCl, 200 mg, 15 eq., DIPEA (348 μL, 20 eq.)) in a nucleophilic substitution to yield H-Nglu(OAll)-Glu-leu-[Cys(2Lut)-Thp-Leu-Ile-Cys]-NH₂ as an intermediate. The sequence assembly was finalize and the allyl ester protection removed. AGLU* was coupled to the four free carboxylic acid functions by treating the peptide resin with the mixture of AGLU* (195 mg, 7.5 eq.), Oxyma (106 mg, 7.5 eq.) and DIC (116 μL, 7.5 eq.) (in 1.7 mL DMF) for 1.5 hours at 50° C. The addition of DIC was repeated and the resin left to agitate for further 90 min at 50° C. Thereafter the steps of ‘Cleavage method B’ where performed to cleave the peptide from the resin. The lyophilized crude peptide residue was subjected to ‘Solution Cyclization method A’ with bis-(bromomethyl)-pyridin as cyclization reagent. After lyophilization the crude product was purified by preparative HPLC (25 to 55% B in 30 min—Kinetex) to yield 30.9 mg of the pure title compound (17.4%). HPLC t_{R}=8.36 min. LC/TOF-MS: exact mass 1776.731 (calculated 1776.729). C₈₁H₁₁₃ClN₁₆O₂₃S₂ (MW=1778.446).

### Example 19: Synthesis of AGLU-Glutar-Asp-Trp-5Clw-Tap-Glu-leu-[Cys(2Lut)-Apc(NOPO)-Leu-Ile-Cys]-APAc-glu(AGLU)-NH₂ (GIP-029)

Initially the C-terminal part (AGLU-Glutar-Asp-Trp-5Clw-Tap-Glu-leu-Cys-Apc(Alloc)-Leu-Ile-Cys-APAc-glu(AGLU)-NH₂) of the linear sequence of the peptide was assembled according to the ‘General procedures for Automated/Semi-automated Solid-Phase Synthesis’ in a 20 μmol scale on a Rink amide resin. The alloc protection group cleaved from the Apc side chain by performing the steps of the ‘Alloc/Allyl-deprotection’. The NOPO chelator was coupled to the Apc side chain by adding a pre-activated solution of NOPO—OH (50 mg, 5 eq.), HATU (76 mg, 10 eq.) and DIPEA (71 μL, 20 eq.) in DMSO (1 mL) to resin. After 90 min at room temperature, the resin was under conditions described in ‘Cleavage method D’. The lyophilized crude peptide residue was subjected to ‘Solution Cyclization method A’ with bis-(bromomethyl)-pyridin as cyclization reagent. After lyophilization the crude product was purified by preparative HPLC (20 to 35% B in 30 min—Kinetex) to yield 12.63 mg of the pure title compound (23.0%). HPLC t_{R}=7.22 min. LC/TOF-MS: exact mass 2740.127 (calculated 2740.129). C₁₁₆H₁₈₁ClN₂₅O₃₉P₃S₂ (MW=2742.333).

### Example 20: Synthesis of Ac-Asp-Trp-5Clw-Hyp-Glu-leu-[Cys(mli)-Thp-Leu-Ile-Hcy]-NH₂ (GIP-361)

The linear sequence (Ac-Asp-Trp-5Clw-Hyp-Glu-leu-Cys-Thp-Leu-Ile-Hcy-NH₂) of the peptide was assembled according to the ‘General procedures for Automated/Semi-automated Solid-Phase Synthesis’ in a 50 μmol scale on a Rink amide resin. Thereafter the steps of ‘Cleavage method B’ where performed to cleave the peptide from the resin. The lyophilized crude peptide residue was subjected to ‘Solution Cyclization method B: Cyclization with Maleimide Derivatives’. After lyophilization the crude product was purified by preparative HPLC (30 to 60% B in 30 min—Kinetex) to yield 11.16 mg of the pure title compound (13.9%). HPLC t_{R}=9.69 min. LC/TOF-MS: exact mass 1601.631 (calculated 1601.609). C₇₃H₉₆ClN₁₅O₂₀S₂ (MW=1603.220).

### Example 21: Synthesis of Ac-Asp-Trp-5Clw-Hyp-Glu-leu-[Hey-Thp-Leu-Ile-Hcy]-NH₂ (GIP-363)

The linear sequence (Ac-Asp-Trp-5Clw-Hyp-Glu-leu-Hcy-Thp-Leu-Ile-Hcy-NH₂) of the peptide was assembled according to the ‘General procedures for Automated/Semi-automated Solid-Phase Synthesis’ in a 50 μmol scale on a Rink amide resin. Thereafter the steps of ‘Cleavage method B’ where performed to cleave the peptide from the resin. The lyophilized crude peptide residue was subjected to ‘Solution Cyclization method C: Disulfide cyclization’. After lyophilization the crude product was purified by preparative HPLC (30 to 60% B in 30 min—Kinetex) to yield 14.19 mg of the pure title compound (18.6%). HPLC t_{R}=10.59 min. LC/TOF-MS: exact mass 1520.639 (calculated 1520.624). C₇₀H₉₇ClN₁₄O₁₈S₂ (MW=1522.190).

### Example 22: Synthesis of Ac-Asp-Trp-5Clw-Hyp-Glu-leu-[Cys(tMeBn(DOTA-AET))-Thp-Leu-Ile-Cys]-NH₂ (GIP-124)

The linear sequence (Ac-Asp-Trp-5Clw-Hyp-Glu-leu-Cys-Thp-Leu-Ile-Cys-NH₂) of the peptide was assembled according to the ‘General procedures for Automated/Semi-automated Solid-Phase Synthesis’ in a 100 μmol scale on a Rink amide resin. Thereafter the steps of ‘Cleavage method B’ where performed to cleave the peptide from the resin. The lyophilized crude peptide residue was dissolved in 120 mL of a 1:1 mixture of ammonium bicarbonate solution (50 mM, pH=8.5) and acetonitrile. 1,3,5-Tris(bromomethyl)benzene (53.5 mg, 1.5 eq compared to initial resin loading) was added to the solution, which was then left to stir for 1 hour. Then 2-aminoethanthiol (77 mg, 10 eq.) was added to the solution, which was stirred for further 5 hours. After addition of TFA (100 μL) the solution was lyophilized and the remainder subjected to HPLC purification (30 to 70% B in 30 min—Kinetex) to yield 41.15 mg (24.6%) of the peptide precursor Ac-Asp-Trp-5Clw-Hyp-Glu-leu-[Cys(tMeBn(H-AET))-Thp-Leu-Ile-Cys]-NH₂. 15 mg of the latter where dissolved in DMSO and the pH of the solution adjusted to 8 by addition of DIPEA (7 μL). DOTA-NHS (10.1 mg, 1.5 eq.) was added and the pH value re-adjusted to 8 by addition of further DIPEA (7 μL). After completion of the reaction the solution was subjected to preparative HPLC (25 to 50% B in 30 min—Kinetex) to yield 12.57 mg of the pure title compound (68.1%—yield for coupling of DOTA—overall yield: 16.7%). HPLC t_{R}=9.53 min. LC/TOF-MS: exact mass 2071.894 (calculated 2071.865). C₉₅H₁₃₄ClN₁₉O₂₅S₃ (MW=2073.848).

### Example 23: Synthesis of DOTA-Ttds-Asp-Trp-5Clw-Pro-Glu-leu-[Cys(3CbBn)-Aib-Leu-Ile-en] (GIP-359)

The linear sequence (DOTA(OtBu)₃-Ttds-Asp(OtBu)-Trp(Boc)-5Clw-Pro-Glu(OtBu)-leu-Cys(Mmt)-Aib-Leu-Ile-en-H) of the protected peptide was assembled according to the ‘General procedures for Automated/Semi-automated Solid-Phase Synthesis’ in a 50 μmol scale on a 1,2-diaminoethane trityl resin. The protected peptide was cleaved from the resin by performing the steps of ‘Cleavage method A’. To the C-terminal 1,2-diaminoethane moiety 3-chloromethyl-benzoic acid was coupled (conditions: 2 eq. 3-chloromethyl-benzoic acid, 2 eq. Oxyma, 2 eq. DIC in DMF/DCM (3:1, 600 μL), overnight). The solvent was evaporated and all protecting groups were removed by execution of ‘Cleavage method C’. The reaction of the previously introduced benzyl chloride and the sulfhydryl group of the cysteine side chain to form the peptide cycle was allowed to proceed overnight in a sodium acetate buffer (pH 8.0). Afterwards, the crude product was isolated from the reaction buffer by a solid phase extraction. The lyophilized crude product was purified by preparative HPLC (30 to 60% B in 30 min—Kinetex) to yield 11.6 mg of the pure title compound (10.8%). HPLC t_{R}=8.50 min. LC/TOF-MS: exact mass 2139.017 (calculated 2139.015). C₁₀₁H₁₄₇ClN₂₀O₂₇S (MW=2140.889).

### Example 24: Synthesis of HO-Succinyl-Trp-5Clw-Hyp-Glu-leu-[Cys(2Lut)-Apc(MeO10kDaPEGAc)-Leu-Ile-Cys]-APAc-Lys(DOTA)-NH₂ (GIP-142)

The linear sequence (tBuO-Succinyl-Trp-5Clw-Hyp-Glu-leu-Cys-Apc-Leu-Ile-Cys-APAc-Lys(Alloc)-NH₂) was assembled according to the ‘General procedures for Automated/Semi-automated Solid-Phase Synthesis’ in a 50 μmol scale on a Rink amide resin. The Alloc-protection group was removed from the Lys side chain by performing the steps of the ‘Alloc/Allyl-deprotection’. DOTA(OtBu)₃-OH was coupled and the peptide cleaved from the resin employing the steps of ‘Cleavage method B’. The lyophilized crude peptide residue was subjected to ‘Solution Cyclization method A’ with bis-(bromomethyl)-pyridin as cyclization reagent. After lyophilization the crude product was purified by preparative HPLC (20 to 50% B in 30 min—Kinetex) to yield 30.8 mg of the cyclic intermediate peptide (28.1%). HPLC t_{R}=8.36 min. LC/TOF-MS: exact mass 2194.019 (calculated 2194.015). C₁₀₂H₁₄₈ClN₂₃O₂₅S₂ (MW=2195.995). 12.5 mg of the intermediate peptide (5.7 μmol) were dissolved in DMSO (600 μL) and the pH-value adjusted to roughly pH=8.0 by addition of DIPEA (2 μL). 59.3 mg Methoxy PEG Succinimidyl Carboxymethyl Ester (5.7 μmol; 10 kDa average mass, JenKem M-SCM-10K) were dissolved in acetonitrile (400 μL) and added to the solution of the peptide. The pH-value was adjusted to pH=8 by addition of small portions (0.5 μL) of DIPEA. After 3 hours the acetonitrile fraction of the reaction solution was evaporated and the remaining solution subjected to preparative HPLC to yield 8.87 mg of the pure title compound (12.7%). HPLC t_{R}=12.30 min. Average MW=12196.

### Example 25: Synthesis of AGLU-Glutar-Asp-Trp-5Clw-Tap-Glu-leu-[Cys(2Lut)-Apc(DOTAM)-Leu-Ile-Cys]-APAc-glu(AGLU)-NH₂ (GIP-370)

The linear sequence of the precursor peptide AGLU-Glutar-Asp-Trp-5Clw-N₃Tap-Glu-leu-Cys-Apc-Leu-Ile-Cys-APAc-glu(AGLU)-NH₂ was assembled according to the ‘General procedures for Automated/Semi-automated Solid-Phase Synthesis’ in a 50 μmol scale on a Rink amide resin and cleaved from the later employing the steps of ‘Cleavage method B’. The lyophilized crude peptide residue was subjected to ‘Solution Cyclization method A’ with bis-(bromomethyl)-pyridin as cyclization reagent. After lyophilization the crude product was purified by preparative HPLC (30 to 50% B in 30 min—Kinetex) to yield 29.95 mg of the cyclic intermediate peptide (26.10%). HPLC t_{R}=6.67 min. LC/TOF-MS: exact mass 2289.003 (calculated 2289.000). C₁₀₂H₁₄₉ClN₂₄O₃₀S₂ (MW=2291.006). The purified intermediate peptide (13 μmol) was dissolved in DMSO (250 μL) and the pH values set to 8.5 by addition of DIPEA. DOTAM-OH (10.5 mg, 26 μmol, 2 eq.), HATU (9.9 mg, 26 μmol, 2 eq.) and DIPEA (9.1 μL, 52 μmol, 4 eq.) were dissolved in DMSO (250 μL). After complete dissolution the mixture was combined with solution of the intermediate peptide. After stirring of the solution for 2.5 hours, a solution of triscarboxylethylenephosphane HCl (53 mg in 150 μL DMSO) was added, the solution stirred for 30 min and then subjected to preparative HPLC (20 to 40% B in 30 min—Kinetex) to yield 12.3 mg of the pure title compound (9.3%). HPLC t_{R}=5.50 min. LC/TOF-MS: exact mass 2626.232 (calculated 2646.238). C₁₁₈H₁₈₀ClN₂₉O₃₄S₂ (MW=2648.455).

### Example 26: Synthesis of NTASEROL-Asp-Trp-5Clw-Tap-Glu-leu-[Cys(2Lut)-Apc(DOTA)-Leu-Ile-Cys]-APAc-dap(NTASEROL)-NH₂ (GIP-373)

The linear sequence of the precursor peptide H-Asp-Trp-5Clw-Tap-Glu-leu-Cys-Apc(Mtt)-Leu-Ile-Cys-APAc-dap(Alloc)-NH₂ was assembled according to the ‘General procedures for Automated/Semi-automated Solid-Phase Synthesis’ in a 100 μmol scale on a Rink amide resin. Nitrilotriacetic acid (153 mg, 800 μmol) and DIC (15.4 μL) were dissolved in DMSO (3.2 mL) and DMF (4.8 mL). After 2 minutes the mixture was added to the resin, which was then agitated for 1 hour. The step was repeated once, to complete the coupling of nitrilotriacetic acid to the N-terminus of the nascent peptide. Then the remaining free carboxylic acid functions of the nitrilotriacetic acid at the N-terminus of the peptide were activated by addition of a 0.4 M HATU solution (750 μL in DMF, 3 eq.) and a 0.9 M DIPEA (345 μL in DMF, 3 eq.). After 2 minutes the solution of 2,2-dimethyl-1,3-dioxan-5-amine (78.8 mg, 6 eq.) in a 0.9 M DIPEA solution (690 μL in DMF, 6 eq.) was added and the resin left to agitation overnight to complete the synthesis of the N-terminal NTASEROL unit. The Alloc-protection group was removed from the dap side chain by performing the steps of the ‘Alloc/Allyl-deprotection’. To the liberated amino function nitrilotriacetic acid was coupled as described above. The synthesis of the NTASEROL unit was completed by coupling 2,2-dimethyl-1,3-dioxan-5-amine to the free carboxylic acid functions of the nitrilotriacetic acid attached to the dap side chain as described above. The Mtt-group was removed from the Apc side chain by performing the ‘Mtt-deprotection’ as described in the ‘General procedures for Automated/Semi-automated Solid-Phase Synthesis’, DOTA(OtBu)₃-OH was coupled and the peptide cleaved from the resin by performing the steps of ‘Cleavage method B’ (4 hours cleavage time). The lyophilized crude peptide residue was subjected to ‘Solution Cyclization method A’ with bis-(bromomethyl)-pyridin as cyclization reagent. After lyophilization the crude product was purified by preparative HPLC (23 to 38% B in 30 min—Kinetex) to yield 5.52 mg of the pure title compound (2.0%). HPLC t_{R}=6.7 min. LC/TOF-MS: exact mass 2804.250 (calculated 2804.271). C₁₂₃H₁₈₆ClN₃₁O₃₈S₂ (MW=2806.568).

### Example 27: Synthesis of NTASEROL-Asp-Trp-5Clw-Tap-Glu-leu-[Cys(2Lut)-Apc(DOTA)-Leu-Ile-Cys]-APAc-GLUSEROL (GIP-374)

After 100 μmol trityl resin was loaded with Fmoc-1-Serinol(tBu)-OH (Serol) and the Fmoc protection of the latter had been removed, the linear sequence of the precursor peptide H-Asp-Trp-5Clw-N₃Tap-Glu-leu-Cys-Apc-Leu-Ile-Cys-APAc-glu(OAll)-Serol was assembled according to the ‘General procedures for Automated/Semi-automated Solid-Phase Synthesis’. Nitrilotriacetic acid (153 mg, 800 μmol) and DIC (15.4 μL) were dissolved in DMSO (3.2 mL) and DMF (4.8 mL). After 2 minutes the mixture was added to the resin, which was then agitated for 1 hour. The step was repeated once, to complete the coupling of nitrilotriacetic acid to the N-terminus of the nascent peptide. The Allyl-protection group was removed from the glu side chain by performing the steps of the ‘Alloc/Allyl-deprotection’. Then the remaining free carboxylic acid functions of the nitrilotriacetic acid at the N-terminus of the peptide and the free carboxylic acid function at the glu side chain were activated by addition of a 0.4 M HATU solution (750 μL in DMF, 3 eq.) and a 0.9 M DIPEA (345 μL in DMF, 3 eq.). After 2 minutes the solution of 2,2-dimethyl-1,3-dioxan-5-amine (78.8 mg, 6 eq.) in a 0.9 M DIPEA solution (690 μL in DMF, 6 eq.) was added and the resin left to agitation overnight to complete the synthesis of the N-terminal NTASEROL unit and the C-terminal GLUSEROL unit. The peptide was cleaved from the resin by performing the steps of ‘Cleavage method B’ (2 hours cleavage time) and after a lyophilization step, the obtained crude material was subjected to ‘Solution Cyclization method A’ with bis-(bromomethyl)-pyridin as cyclization reagent. After lyophilization the crude product was purified by preparative HPLC (25 to 45% B in 30 min—Kinetex) to yield 15.72 mg of the cyclic intermediate peptide (6.8%). HPLC t_{R}=7.73 min. LC/TOF-MS: exact mass 2315.009 (calculated 2315.027). C₁₀₃H₁₅₁ClN₂₆O₂₉S₂ (MW=2317.047). The isolated pure peptide precursor (6.8 μmol) was dissolved in DMSO (200 μL), DOTA-NHS (10.2 μmol, 1.5 eq.) added, and the pH value of the solution adjusted to pH=8 by addition of DIPEA. After stirring for 30 minutes, a solution of triscarboxylethylenephosphane HCl (53 mg in 150 μL DMSO) was added, the solution stirred for 30 min and then subjected to preparative HPLC (20 to 40% B in 30 min—Kinetex) to yield 8.62 mg of the pure title compound (3.2%). HPLC t_{R}=6.60 min. LC/TOF-MS: exact mass 2675.217 (calculated 2675.208). C₁₁₉H₁₇₉ClN₂₈O₃₆S₂ (MW=2677.450).

### Example 28: Preparation of Transition Metal Complexes of Compounds of the Invention

A. General Procedure for the Preparation of a Peptide Comprising Chelator-Transition Metal-Complexes from Corresponding Peptides Comprising Uncomplexed Chelator

A 0.1 mM solution of the peptide comprised by uncomplexed Chelator in
0.4 M sodium acetate, pH=5 (Buffer A) (in case of In(III), Lu(III) or Ga(III) complexes) or
0.1 M ammonium acetate, pH=8 (Buffer B) (in case of Eu(III) complexes)
   was diluted with a solution 0.1 mM solution of the corresponding metal salt in water whereby the molar ratio of peptide to metal was adjusted to 1:3. The solution was stirred
at 50° C. for 20 min (also referred to herein as Condition A) (in case of In(III), Lu(III) or Ga(III) complexes)
or
at room temperature overnight (also referred to herein as Condition B) (in case of Eu(III) complexes).

The solution was then applied to
HPLC purification (also referred to herein as Purification A)
or
solid phase extraction (also referred to herein as Purification B).

In either case (HPLC purification or solid phase extraction) fractions containing the pure product were pooled and freeze dried.

### B. Indium-Complex of AGLU-Glutar-Asp-Trp-5Clw-Hyp-Glu-leu-[Cys(2Lut)-Apc(DOTA)-Leu-Ile-Cys]-APAc-glu(AGLU)-NH₂ (GIP-065)

The complex was prepared starting from 15 mg peptide (5.7 μmol) dissolved in Buffer A, diluted with a solution of InCl₃×4H₂O which was treated with Condition A. In the purification step ‘Purification B’ was employed to yield 12.85 mg of the pure title compound (82.3%). HPLC t_{R}=7.13 min. LC/TOF-MS: exact mass 2760.063 (calculated 2762.054). C₁₁₈H₁₇₃ClInN₂₅O₃₈S₂ (MW=2764.189).

### C. Lutetium-Complex of AGLU-Glutar-Asp-Trp-5Clw-Hyp-Glu-leu-[Cys(2Lut)-Apc(DOTA)-Leu-Ile-Cys]-APAc-glu(AGLU)-NH₂ (GIP-066)

The complex was prepared starting from 15 mg peptide (5.7 μmol) dissolved in Buffer A, diluted with a solution of LuCl₃ which was treated with Condition A. In the purification step ‘Purification B’ was employed to yield 10.87 mg of the pure title compound (68.10%). HPLC t_{R}=7.15 min. LC/TOF-MS: exact mass 2822.092 (calculated 2822.091). C₁₁₈H₁₇₃ClLuN₂₅O₃₈S₂ (MW=2824.337).

### D. Gallium-Complex of AGLU-Glutar-Asp-Trp-5Clw-Tap-Glu-leu-[Cys(2Lut)-Apc(DOTA)-Leu-Ile-Cys]-APAc-glu(AGLU)-NH₂ (GIP-069)

The complex was prepared starting from 25 mg peptide (9.5 μmol) dissolved in Buffer A, diluted with a solution of Ga(NO₃)₃×H₂O which was treated with Condition A. In the purification step ‘Purification B’ to yield 16.42 mg of the pure title compound (63.6%). HPLC t_{R}=6.91 min. LC/TOF-MS: exact mass 2715.104 (calculated 2715.092). C₁₁₈H₁₇₄ClGaN₂₆O₃₇S₂ (MW=2718.109).

### E. Europium-Complex of DOTA-glu(AGLU)-APAc-Asp-Trp-5Clw-Hyp-Glu-leu-[Cys(2Lut)-Thp-Leu-Ile-Cys]-APAc-glu(AGLU)-NH₂ (GIP-064)

The complex was prepared starting from 6 mg peptide (2.1 μmol) dissolved in Buffer B, diluted with a solution of EuCl₃×6H₂O which was treated with Condition B. In the purification step ‘Purification B’ was employed to yield 4.88 mg of the pure title compound (77.1%). HPLC t_{R}=6.80 min. LC/TOF-MS: exact mass 2954.158 (calculated 2956.161). C₁₂₅H₁₈₅ClEuN₂₇O₄₀S₂ (MW=2957.518).

### E. Gallium-Complex Synthesis of AGLU-Glutar-Asp-Trp-5Clw-Tap-Glu-leu-[Cys(2Lut)-Apc(NODAGA)-Leu-Ile-Cys]-APAc-glu(AGLU)-NH₂ (GIP-071)

The complex was prepared starting from 8 mg peptide (3 μmol) dissolved in Buffer A, diluted with a solution of Ga(NO₃)₃×H₂O which was treated with Condition A. In the purification step ‘Purification B’ was employed to yield 6.16 mg of the pure title compound (75.10%). HPLC t_{R}=6.93 min. LC/TOF-MS: exact mass 2686.071 (calculated 2686.065). C₁₁₇H₁₇₁ClGaN₂₅O₃₇S₂ (MW=2689.068).

### Example 29: FACS Binding Assay

In order to determine binding of compounds according to the present invention to GIPR-expressing cells, a FACS binding assay was established.

CHO-K1-hGIPR cells (GenScript Biotech, #M00486) were cultured in Ham's F12 medium (Sigma-Aldrich, #N4888) including 10% fetal calf serum (FCS), 2 mM L-glutamine, 100 U/mL penicillin, 100 μg/mL streptomycin, 200 μg/mL Zeocin and 100 μg/mL HygromycinB. Cells were detached with Accutase (Biolegend, #BLD-423201) and washed in FACS buffer (PBS including 1% FCS). Cells were diluted in FACS buffer to a final concentration of 500.000 cells per mL. 200 μL of the cell suspension were transferred to a u-shaped non-binding 96-well plate (Greiner) and cells were washed in ice-cold FACS buffer.

For EC₅₀ determination, cells were incubated with various concentrations of biotinylated compound (GIP-057) at 4° C. for 1 hour. The determined pEC₅₀ value of GIP-057 was 8.5. For IC₅₀ determination, cells were incubated with 20 nM GIP-057 (H-Met-Tyr-Asp-Trp-Trp-Pro-Glu-Leu-[Cys(3MeBn)-Ala-Leu-Ile-Cys]-Ttds-Lys(Bio)-NH₂) in the presence of increasing concentrations of non-labeled test compounds at 4° C. for 1 hour. Cells were washed twice in FACS buffer. An additional incubation step with 1 μg/mL APC-Streptavidin (Miltenyi; #130-106-791) in 50 μL FACS buffer for 30 min on ice was performed followed by two washes with FACS buffer.

Cells were analyzed in an Attune NxT flow cytometer. Median fluorescence intensities (MFI) of the APC/Cy5-channel were calculated by Attune NxT software. MFI values were plotted against peptide concentration and four parameter logistic (4PL) curve fitting and EC₅₀/pEC₅₀ or IC₅₀/pIC₅₀ calculations were performed using ActivityBase software.

The results of the IC₅₀ assay is shown in Table 8. pIC₅₀ category A stands for pIC₅₀ values>8.0, category B for pIC₅₀ values between 7.1 and 8.0, category C for pIC₅₀ values between 6.1 and 7.0, and category D for pIC₅₀ values≤6.0.

TABLE 8
Compound ID, sequence, exact calculated mass, exact mass found, retention time in
min as determined by HPLC and pIC₅₀ category of FACS binding assay.

| | | Exact | Exact | Rt | |
|---|---|---|---|---|---|
| Compound | | Mass | Mass | (HPLC) | pIC₅₀ |
| ID | Sequence | (Calc) | (Found) | [min] | Category |
| GIP-001 | AGLU-Glutar-Asp-Trp-5Clw-Tap-Glu-leu- | 2649.190 | 2649.198 | 6.6 | A |
| | [Cys(2Lut)-Apc(DOTA)-Leu-Ile-Cys]-APAc- | | | | |
| | glu(AGLU)-NH₂ | | | | |
| GIP-002 | AGLU-Glutar-Asp-Trp-5Clw-Hyp-Glu-leu- | 2650.174 | 2650.178 | 7.1 | A |
| | [Cys(2Lut)-Apc(DOTA)-Leu-Ile-Cys]-APAc- | | | | |
| | glu(AGLU)-NH₂ | | | | |
| GIP-003 | DOTA-glu(AGLU)-APAc-Asp-Trp-5Clw-Hyp-Glu- | 3082.396 | 3082.398 | 6.5 | A |
| | leu-[Cys(2Lut)-Apc(AGLU-Glutar)-Leu-Ile-Cys]- | | | | |
| | APAc-glu(AGLU)-NH₂ | | | | |
| GIP-004 | AGLU-Glutar-Asp-Trp-5Clw-Hyp-Glu-leu- | 2779.253 | 2779.260 | 7.0 | A |
| | [Cys(2Lut)-Thp-Leu-Ile-Cys]-APAc-glu(AGLU)- | | | | |
| | Lys(DOTA)-NH₂ | | | | |
| GIP-005 | DOTA-glu(AGLU)-APAc-Asp-Trp-5Clw-Hyp-Glu- | 2806.264 | 2806.273 | 6.7 | A |
| | leu-[Cys(2Lut)-Thp-Leu-Ile-Cys]-APAc- | | | | |
| | glu(AGLU)-NH₂ | | | | |
| GIP-006 | AGLU-Glutar-APAc-Asp-Trp-5Clw-Tap(AGLU- | 3066.401 | 3066.416 | 6.3 | A |
| | Glutar)-Glu-leu-[Cys(2Lut)-Apc(DOTA)-Leu-Ile- | | | | |
| | Cys]-APAc-glu(AGLU)-NH₂ | | | | |
| GIP-007 | AGLU-Glutar-APAc-Asp-Trp-5Clw-Tap(AGLU- | 3358.528 | 3358.542 | 6.2 | B |
| | Glutar)-Glu-leu-[Cys(2Lut)-Apc(DOTA- | | | | |
| | glu(AGLU))-Leu-Ile-Cys]-APAc-glu(AGLU)-NH2 | | | | |
| GIP-008 | AGLU-Glutar-APAc-Asp-Trp-5Clw-Tap-Glu-leu- | 3081.412 | 3081.423 | 6.1 | A |
| | [Cys(2Lut)-Apc(DOTA-glu(AGLU))-Leu-Ile-Cys]- | | | | |
| | APAc-glu(AGLU)-NH₂ | | | | |
| GIP-009 | DOTA-glu(AGLU)-O2Oc-Asp-Trp-5Clw-Tap-Glu- | 3086.391 | 3086.411 | 6.7 | A |
| | leu-[Cys(2Lut)-Apc(AGLU-Glutar)-Leu-Ile-Cys]- | | | | |
| | APAc-glu(AGLU)-NH₂ | | | | |
| GIP-010 | AGLU-Glutar-Asp-Trp-5Clw-Hyp-Glu-leu- | 2763.258 | 2763.274 | 7.2 | A |
| | [Cys(2Lut)-Apc(AGLU-Glutar)-Leu-Ile-Cys]- | | | | |
| | APAc-Lys(DOTA)-NH₂ | | | | |
| GIP-011 | AGLU-Glutar-APAc-Asp-Trp-5Clw-Hyp-Glu-leu- | 2790.269 | 2790.274 | 6.6 | A |
| | [Cys(2Lut)-Apc(DOTA)-Leu-Ile-Cys]-APAc- | | | | |
| | glu(AGLU)-NH₂ | | | | |
| GIP-012 | DOTA-APAc-Asp-Trp-5Clw-Hyp-Glu-leu- | 2790.269 | 2790.270 | 6.8 | A |
| | [Cys(2Lut)-Apc(AGLU-Glutar)-Leu-Ile-Cys]- | | | | |
| | APAc-glu(AGLU)-NH₂ | | | | |
| GIP-013 | AGLU-Glutar-APAc-Asp-Trp-5Clw-Tap-Glu-leu- | 2789.285 | 2789.309 | 6.6 | A |
| | [Cys(2Lut)-Apc(DOTA)-Leu-Ile-Cys]-APAc- | | | | |
| | glu(AGLU)-NH₂ | | | | |
| GIP-014 | AGLU-Glutar-APAc-Asp-Trp-5Brw-Tap-Glu-leu- | 2833.234 | 2833.261 | 6.7 | A |
| | [Cys(2Lut)-Apc(DOTA)-Leu-Ile-Cys]-APAc- | | | | |
| | glu(AGLU)-NH₂ | | | | |
| GIP-015 | AGLU-Glutar-Asp-Trp-5Brw-Tap-Glu-leu- | 2693.139 | 2693.131 | 6.8 | A |
| | [Cys(2Lut)-Apc(DOTA)-Leu-Ile-Cys]-APAc- | | | | |
| | glu(AGLU)-NH₂ | | | | |
| GIP-016 | AGLU-Glutar-Hse-Trp-5Brw-Tap-Glu-leu- | 2679.160 | 2679.150 | 6.7 | A |
| | [Cys(2Lut)-Apc(DOTA)-Leu-Ile-Cys]-APAc- | | | | |
| | glu(AGLU)-NH₂ | | | | |
| GIP-017 | DOTA-glu(AGLU)-Asp-Trp-5Clw-Tap-Glu-leu- | 2665.185 | 2665.197 | 6.7 | A |
| | [Cys(2Lut)-Thp-Leu-Ile-Cys]-APAc-glu(AGLU)- | | | | |
| | NH₂ | | | | |
| GIP-018 | DOTA-O20c-glu(AGLU)-Asp-Trp-5Clw-Tap-Glu- | 2810.259 | 2810.260 | 6.7 | A |
| | leu-[Cys(2Lut)-Thp-Leu-Ile-Cys]-APAc- | | | | |
| | glu(AGLU)-NH₂ | | | | |
| GIP-019 | DOTA-glu(AGLU)-PEG6-Asp-Trp-5Clw-Tap-Glu- | 3000.379 | 3000.379 | 7.0 | A |
| | leu-[Cys(2Lut)-Thp-Leu-Ile-Cys]-APAc- | | | | |
| | glu(AGLU)-NH₂ | | | | |
| GIP-020 | DOTA-glu(AGLU)-O2Oc-Asp-Trp-5Clw-Tap-Glu- | 2810.259 | 2810.247 | 6.9 | A |
| | leu-[Cys(2Lut)-Thp-Leu-Ile-Cys]-APAc- | | | | |
| | glu(AGLU)-NH₂ | | | | |
| GIP-021 | HO-Succinyl-Trp-5Clw-Hyp-Glu-leu-[Cys(2Lut)- | 2487.126 | 2487.135 | 7.7 | A |
| | Thp-Leu-Ile-Cys]-APAc-glu(AGLU)-Lys(DOTA)- | | | | |
| | NH₂ | | | | |
| GIP-022 | HO-Succinyl-Trp-5Clw-Tap-Glu-leu-[Cys(2Lut)- | 2485.158 | 2485.165 | 7.0 | A |
| | Apc-Leu-Ile-Cys]-APAc-glu(AGLU)-Lys(DOTA)- | | | | |
| | NH₂ | | | | |
| GIP-023 | HO-Succinyl-Trp-5Clw-Tap-Glu-leu-[Cys(2Lut)- | 2357.063 | 2357.080 | 7.3 | A |
| | Apc(DOTA)-Leu-Ile-Cys]-APAc-glu(AGLU)-NH₂ | | | | |
| GIP-024 | AGLU-Glutar-Asp-Trp-5Clw-Tap-Glu-leu- | 2651.205 | 2651.200 | 6.8 | A |
| | [Cys(2Lut)-Lys(DOTA)-Leu-Ile-Cys]-APAc- | | | | |
| | glu(AGLU)-NH₂ | | | | |
| GIP-025 | AGLU-Glutar-Asp-Trp-5Clw-Tap-Glu-leu- | 2651.205 | 2651.194 | 6.5 | B |
| | [Cys(2Lut)-lys(DOTA)-Leu-Ile-Cys]-APAc- | | | | |
| | glu(AGLU)-NH₂ | | | | |
| GIP-026 | AGLU-Glutar-Asp-Trp-5Clw-Tap-Glu-leu- | 2623.174 | 2623.163 | 6.7 | A |
| | [Cys(2Lut)-Dab(DOTA)-Leu-Ile-Cys]-APAc- | | | | |
| | glu(AGLU)-NH₂ | | | | |
| GIP-027 | AGLU-Glutar-Asp-Trp-5Clw-Tap-Glu-leu- | 2623.174 | 2623.168 | 6.4 | B |
| | [Cys(2Lut)-dab(DOTA)-Leu-Ile-Cys]-APAc- | | | | |
| | glu(AGLU)-NH₂ | | | | |
| GIP-028 | AGLU-Glutar-Asp-Trp-5Clw-Tap-Glu-leu- | 2620.163 | 2620.134 | 6.7 | A |
| | [Cys(2Lut)-Apc(NODAGA)-Leu-Ile-Cys]-APAc- | | | | |
| | glu(AGLU)-NH₂ | | | | |
| GIP-029 | AGLU-Glutar-Asp-Trp-5Clw-Tap-Glu-leu- | 2740.129 | 2740.127 | 7.2 | A |
| | [Cys(2Lut)-Apc(NOPO)-Leu-Ile-Cys]-APAc- | | | | |
| | glu(AGLU)-NH₂ | | | | |
| GIP-030 | AGLU-Glutar-Asp-Trp-5Clw-Tap-Glu-leu- | 2449.158 | 2449.225 | 6.4 | A |
| | [Cys(2Lut)-Apc(N4Ac)-Leu-Ile-Cys]-APAc- | | | | |
| | glu(AGLU)-NH₂ | | | | |
| GIP-031 | AGLU-Glutar-Asp-Trp-5Clw-Tap-Glu-leu- | 2677.185 | 2677.281 | 7.1 | A |
| | [Cys(2Lut)-Apc(NODAGA-Gly)-Leu-Ile-Cys]- | | | | |
| | APAc-glu(AGLU)-NH₂ | | | | |
| GIP-033 | AGLU-Glutar-APAc-Asp-Trp-5Clw-Hyp-Glu-leu- | 2650.174 | 2650.186 | 6.6 | A |
| | [Cys(2Lut)-Apc(DOTA)-Leu-Ile-Cys]-glu(AGLU)- | | | | |
| | NH₂ | | | | |
| GIP-034 | AGLU-Glutar-Hse-Trp-5Clw-Tap-Glu-leu- | 2635.211 | 2635.203 | 6.6 | A |
| | [Cys(2Lut)-Apc(DOTA)-Leu-Ile-Cys]-APAc- | | | | |
| | glu(AGLU)-NH₂ | | | | |
| GIP-035 | AGLU-Glutar-Asp-Trp-5Clw-Tap-Glu-Leu- | 2649.190 | 2649.214 | 7.3 | A |
| | [Cys(2Lut)-Apc(DOTA)-Leu-Ile-Cys]-APAc- | | | | |
| | glu(AGLU)-NH₂ | | | | |
| GIP-036 | DOTA-glu(AGLU)-Asp-Trp-5Clw-Hyp-Glu-leu- | 2666.169 | 2666.180 | 7.1 | A |
| | [Cys(2Lut)-Thp-Leu-Ile-Cys]-APAc-glu(AGLU)- | | | | |
| | NH₂ | | | | |
| GIP-037 | DOTA-PEG6-glu(AGLU)-Asp-Trp-5Clw-Tap-Glu- | 3000.379 | 3000.379 | 6.8 | A |
| | leu-[Cys(2Lut)-Thp-Leu-Ile-Cys]-APAc- | | | | |
| | glu(AGLU)-NH₂ | | | | |
| GIP-038 | AGLU-Glutar-Asp-Trp-5Clw-Tap-Glu-leu- | 2649.190 | 2649.227 | 7.0 | A |
| | [Cys(2Lut)-Apc(DOTA)-Leu-Ile-cys]-APAc- | | | | |
| | glu(AGLU)-NH₂ | | | | |
| GIP-039 | AGLU-Glutar-APAc-Asp-Trp-5Clw-Hyp-Glu-leu- | 4157.122 | 4157.127 | 7.7 | B |
| | [Cys(2Lut)-Apc(DOTA)-Leu-Ile-Cys]- | | | | |
| | lys(MeO36PEG)-NH₂ | | | | |
| GIP-040 | MeO36PEG-APAc-Asp-Trp-5Clw-Hyp-Glu-leu- | 4044.038 | 4044.039 | 7.5 | B |
| | [Cys(2Lut)-Apc(DOTA)-Leu-Ile-Cys]-glu(AGLU)- | | | | |
| | NH₂ | | | | |
| GIP-041 | MeO36PEG-APAc-Asp-Trp-5Clw-Hyp-Glu-leu- | 4184.133 | 4184.152 | 7.9 | B |
| | [Cys(2Lut)-Apc(DOTA)-Leu-Ile-Cys]-APAc- | | | | |
| | glu(AGLU)-NH₂ | | | | |
| GIP-042 | MeO36PEG-APAc-Asp-Trp-5Clw-Tap-Glu-leu- | 4183.149 | 4183.165 | 7.5 | B |
| | [Cys(2Lut)-Apc(DOTA)-Leu-Ile-Cys]-APAc- | | | | |
| | glu(AGLU)-NH₂ | | | | |
| GIP-043 | AGLU-Glutar-APAc-Asp-Trp-5Clw-Hyp-Glu-leu- | 3082.396 | 3082.412 | 6.9 | A |
| | [Cys(2Lut)-Apc(DOTA-glu(AGLU))-Leu-Ile-Cys]- | | | | |
| | APAc-glu(AGLU)-NH₂ | | | | |
| GIP-044 | DOTA-O20c-glu(AGLU)-Asp-Trp-5Clw-Tap-Glu- | 3086.391 | 3086.414 | 6.6 | A |
| | leu-[Cys(2Lut)-Apc(AGLU-Glutar)-Leu-Ile-Cys]- | | | | |
| | APAc-glu(AGLU)-NH₂ | | | | |
| GIP-045 | DOTA-glu(AGLU)-Asp-Trp-5Clw-Tap-Glu-leu- | 2941.317 | 2941.324 | 6.5 | A |
| | [Cys(2Lut)-Apc(AGLU-Glutar)-Leu-Ile-Cys]- | | | | |
| | APAc-glu(AGLU)-NH₂ | | | | |
| GIP-046 | AGLU-Glutar-APAc-Ser-Trp-5Clw-Tap(AGLU- | 3010.411 | 3010.414 | 6.4 | B |
| | Glutar)-Hse-leu-[Cys(2Lut)-Apc(DOTA)-Leu-Ile- | | | | |
| | Cys]-APAc-glu(AGLU)-NH₂ | | | | |
| GIP-047 | AGLU-Glutar-APAc-Hse-Trp-5Clw-Hyp-Glu-leu- | 3068.417 | 3068.435 | 6.8 | B |
| | [Cys(2Lut)-Apc(DOTA-glu(AGLU))-Leu-Ile-Cys]- | | | | |
| | APAc-glu(AGLU)-NH₂ | | | | |
| GIP-048 | AGLU-Glutar-APAc-Ser-Trp-5Clw-Tap-Hse-leu- | 2733.295 | 2733.307 | 6.2 | B |
| | [Cys(2Lut)-Apc(DOTA)-Leu-Ile-Cys]-APAc- | | | | |
| | glu(AGLU)-NH₂ | | | | |
| GIP-049 | AGLU-Glutar-APAc-Hse-Trp-5Clw-Hyp-Glu-leu- | 2776.290 | 2776.322 | 6.9 | B |
| | [Cys(2Lut)-Apc(DOTA)-Leu-Ile-Cys]-APAc- | | | | |
| | glu(AGLU)-NH₂ | | | | |
| GIP-050 | AGLU-Glutar-APAc-Hse-Trp-5Clw-Tap-Glu-leu- | 2775.306 | 2775.316 | 6.5 | B |
| | [Cys(2Lut)-Apc(DOTA)-Leu-Ile-Cys]-APAc- | | | | |
| | glu(AGLU)-NH₂ | | | | |
| GIP-051 | AGLU-Glutar-Asp-Trp-5Clw-Tap-Glu-leu- | 2649.190 | 2649.232 | 7.0 | B |
| | [cys(2Lut)-Apc(DOTA)-Leu-Ile-Cys]-APAc- | | | | |
| | glu(AGLU)-NH₂ | | | | |
| GIP-052 | AGLU-Glutar-Asp-Trp-5Clw-Tap-Glu-leu- | 2649.190 | 2649.226 | 6.6 | B |
| | [cys(2Lut)-Apc(DOTA)-Leu-Ile-cys]-APAc- | | | | |
| | glu(AGLU)-NH₂ | | | | |
| GIP-053 | DOTA-Ttds-Asp-Trp-5Clw-Pro-Glu-leu- | 2569.204 | 2569.215 | 8.5 | A |
| | [Cys(3MeBn)-Apc(DOTA)-Leu-Ile-AET] | | | | |
| GIP-054 | DOTA-Ttds-Asp-Trp-5Clw-Hyp-Glu-leu- | 2758.279 | 2758.351 | 7.9 | A |
| | [Cys(2Lut)-Thp-Leu-Ile-Cys]-Lys(DOTA)-NH₂ | | | | |
| GIP-055 | DOTA-Ttds-Asp-Trp-5Clw-Hyp-Glu-leu- | 3143.475 | 3143.537 | 7.8 | A |
| | [Cys(2Lut)-Apc(DOTA)-Leu-Ile-Cys]-Lys(DOTA)- | | | | |
| | NH₂ | | | | |
| GIP-056 | DOTA-Ttds-Asp-Trp-5Clw-Tap(DOTA)-Glu-leu- | 3528.671 | 3528.788 | 7.4 | A |
| | [Cys(2Lut)-Apc(DOTA)-Leu-Ile-Cys]-Lys(DOTA)- | | | | |
| | NH₂ | | | | |
| GIP-059 | AGLU-Glutar-APAc-Asp-Trp-5Clw-Tap-Glu-leu- | 3147.314 | 3147.320 | 6.4 | A |
| | [Cys(2Lut)-Apc(GaDOTA-glu(AGLU))-Leu-Ile- | | | | |
| | Cys]-APAc-glu(AGLU)-NH₂ | | | | |
| GIP-060 | AGLU-Glutar-APAc-Asp-Trp-5Clw-Tap-Glu-leu- | 3191.292 | 3191.303 | 6.7 | A |
| | [Cys(2Lut)-Apc(InDOTA-glu(AGLU))-Leu-Ile- | | | | |
| | Cys]-APAc-glu(AGLU)-NH₂ | | | | |
| GIP-061 | AGLU-Glutar-APAc-Asp-Trp-5Clw-Tap-Glu-leu- | 3253.329 | 3253.341 | 6.5 | A |
| | [Cys(2Lut)-Apc(LuDOTA-glu(AGLU))-Leu-Ile- | | | | |
| | Cys]-APAc-glu(AGLU)-NH₂ | | | | |
| GIP-062 | LuDOTA-glu(AGLU)-APAc-Asp-Trp-5Clw-Hyp- | 2978.181 | 2978.182 | 6.8 | A |
| | Glu-leu-[Cys(2Lut)-Thp-Leu-Ile-Cys]-APAc- | | | | |
| | glu(AGLU)-NH₂ | | | | |
| GIP-063 | InDOTA-glu(AGLU)-APAc-Asp-Trp-5Clw-Hyp- | 2916.144 | 2916.143 | 6.8 | A |
| | Glu-leu-[Cys(2Lut)-Thp-Leu-Ile-Cys]-APAc- | | | | |
| | glu(AGLU)-NH₂ | | | | |
| GIP-064 | EuDOTA-glu(AGLU)-APAc-Asp-Trp-5Clw-Hyp- | 2954.160 | 2954.158 | 6.8 | A |
| | Glu-leu-[Cys(2Lut)-Thp-Leu-Ile-Cys]-APAc- | | | | |
| | glu(AGLU)-NH₂ | | | | |
| GIP-065 | AGLU-Glutar-Asp-Trp-5Clw-Hyp-Glu-leu- | 2760.054 | 2760.063 | 7.1 | A |
| | [Cys(2Lut)-Apc(InDOTA)-Leu-Ile-Cys]-APAc- | | | | |
| | glu(AGLU)-NH₂ | | | | |
| GIP-066 | AGLU-Glutar-Asp-Trp-5Clw-Hyp-Glu-leu- | 2822.091 | 2822.092 | 7.2 | A |
| | [Cys(2Lut)-Apc(LuDOTA)-Leu-Ile-Cys]-APAc- | | | | |
| | glu(AGLU)-NH₂ | | | | |
| GIP-067 | AGLU-Glutar-Asp-Trp-5Clw-Tap-Glu-leu- | 2821.107 | 2821.116 | 6.8 | A |
| | [Cys(2Lut)-Apc(LuDOTA)-Leu-Ile-Cys]-APAc- | | | | |
| | glu(AGLU)-NH₂ | | | | |
| GIP-068 | AGLU-Glutar-Asp-Trp-5Clw-Tap-Glu-leu- | 2759.070 | 2759.003 | 7.2 | A |
| | [Cys(2Lut)-Apc(InDOTA)-Leu-Ile-Cys]-APAc- | | | | |
| | glu(AGLU)-NH₂ | | | | |
| GIP-069 | AGLU-Glutar-Asp-Trp-5Clw-Tap-Glu-leu- | 2715.092 | 2715.104 | 6.9 | A |
| | [Cys(2Lut)-Apc(GaDOTA)-Leu-Ile-Cys]-APAc- | | | | |
| | glu(AGLU)-NH₂ | | | | |
| GIP-070 | AGLU-Glutar-Asp-Trp-5Clw-Hyp-Glu-leu- | 2716.076 | 2716.087 | 7.5 | A |
| | [Cys(2Lut)-Apc(GaDOTA)-Leu-Ile-Cys]-APAc- | | | | |
| | glu(AGLU)-NH₂ | | | | |
| GIP-071 | AGLU-Glutar-Asp-Trp-5Clw-Tap-Glu-leu- | 2686.065 | 2686.071 | 6.9 | A |
| | [Cys(2Lut)-Apc(GaNODAGA)-Leu-Ile-Cys]- | | | | |
| | APAc-glu(AGLU)-NH₂ | | | | |
| GIP-072 | AGLU-Glutar-Asp-Trp-5Clw-Hyp-Glu-leu- | 2174.946 | 2174.966 | 7.8 | A |
| | [Cys(2Lut)-Apc(DOTA)-Leu-Ile-AET] | | | | |
| GIP-073 | HO-Succinyl-Trp-5Clw-Hyp-Glu-leu-[Cys(2Lut)- | 2471.131 | 2471.139 | 7.8 | A |
| | Apc(AGLU-Glutar)-Leu-Ile-Cys]-APAc- | | | | |
| | Lys(DOTA)-NH₂ | | | | |
| GIP-074 | Ac-Asp-Trp-1Ni-Pro-Glu-Leu-[Cys(3MeBn)-Aib- | 2332.152 | 2332.163 | 9.9 | A |
| | Leu-Ile-Cys]-Ttds-Lys(DOTA)-NH₂ | | | | |
| GIP-075 | Ac-Asp-Trp-Bta-Pro-Glu-Leu-[Cys(3MeBn)-Aib- | 2338.109 | 2338.116 | 9.9 | A |
| | Leu-Ile-Cys]-Ttds-Lys(DOTA)-NH₂ | | | | |
| GIP-076 | Ac-Asp-Trp-5Clw-Pro-Glu-Leu-[Cys(3MeBn)- | 2355.109 | 2355.114 | 9.5 | A |
| | Aib-Leu-Ile-Cys]-Ttds-Lys(DOTA)-NH₂ | | | | |
| GIP-077 | DOTA-Ttds-Asp-Trp-5Clw-Pro-Glu-leu- | 2141.997 | 2142.002 | 9.2 | A |
| | [Cys(3MeBn)-Aib-Leu-Ile-AET] | | | | |
| GIP-078 | Ac-Asp-Trp-1Ni-Pro-Glu-leu-[Cys(3MeBn)-Aib- | 2332.152 | 2332.157 | 9.3 | A |
| | Leu-Ile-Cys]-Ttds-Lys(DOTA)-NH₂ | | | | |
| GIP-079 | Ac-Asp-Trp-5Clw-Pro-Glu-leu-[Cys(3MeBn)- | 2355.109 | 2355.113 | 9.1 | A |
| | Aib-Leu-Ile-Cys]-Ttds-Lys(DOTA)-NH₂ | | | | |
| GIP-080 | Ac-Asp-Trp-1Ni-Pro-Glu-leu-[Cys(3MeBn)-Egz- | 2372.184 | 2372.206 | 9.5 | A |
| | Leu-Ile-Cys]-Ttds-Lys(DOTA)-NH₂ | | | | |
| GIP-081 | Ac-Asp-Trp-5Fw-Pro-Glu-leu-[Cys(3MeBn)-Aib- | 2339.138 | 2339.144 | 9.2 | A |
| | Leu-Ile-Cys]-Ttds-Lys(DOTA)-NH₂ | | | | |
| GIP-082 | Ac-Asp-Trp-5Brw-Pro-Glu-leu-[Cys(3MeBn)- | 2399.058 | 2399.066 | 9.5 | A |
| | Aib-Leu-Ile-Cys]-Ttds-Lys(DOTA)-NH₂ | | | | |
| GIP-083 | Ac-Asp-5Fw-1Ni-Pro-Glu-leu-[Cys(3MeBn)-Aib- | 2350.143 | 2350.142 | 9.6 | A |
| | Leu-Ile-Cys]-Ttds-Lys(DOTA)-NH₂ | | | | |
| GIP-084 | Ac-Asp-6Fw-1Ni-Pro-Glu-leu-[Cys(3MeBn)-Aib- | 2350.143 | 2350.141 | 9.5 | A |
| | Leu-Ile-Cys]-Ttds-Lys(DOTA)-NH₂ | | | | |
| GIP-085 | HO-Succinyl-Trp-1Ni-Pro-Glu-Leu- | 2275.131 | 2275.133 | 10.4 | A |
| | [Cys(3MeBn)-Aib-Leu-Ile-Cys]-Ttds-Lys(DOTA)- | | | | |
| | NH₂ | | | | |
| GIP-086 | DOTA-Ttds-Asp-Trp-5Clw-Pro-Glu-leu- | 2182.029 | 2182.034 | 9.7 | A |
| | [Cys(3MeBn)-Egz-Leu-Ile-AET] | | | | |
| GIP-087 | DOTA-Ttds-Asp-Trp-5Clw-Pro-Glu-leu- | 2184.008 | 2184.017 | 9.3 | A |
| | [Cys(3MeBn)-Thp-Leu-Ile-AET] | | | | |
| GIP-088 | DOTA-Ttds-Asp-Trp-5Clw-Pro-Glu-leu- | 2183.024 | 2183.034 | 8.8 | A |
| | [Cys(3MeBn)-Apc-Leu-Ile-AET] | | | | |
| GIP-089 | DOTA-APAc-Asp-Trp-5Clw-Pro-Glu-leu- | 1979.908 | 1979.915 | 8.8 | A |
| | [Cys(3MeBn)-Aib-Leu-Ile-AET] | | | | |
| GIP-090 | DOTA-Ttds-Asp-Trp-5Clw-Pro-Glu-leu- | 2142.993 | 2143.001 | 8.0 | A |
| | [Cys(2Lut)-Aib-Leu-Ile-AET] | | | | |
| GIP-091 | DOTA-Ttds-Asp-Trp-5Clw-Pro-Glu-leu- | 2142.993 | 2143.002 | 7.6 | A |
| | [Cys(3Lut)-Aib-Leu-Ile-AET] | | | | |
| GIP-092 | DOTA-Ttds-Asp-Trp-5Clw-Pro-Glu-leu- | 2125.036 | 2125.043 | 8.0 | A |
| | [Dap(3MeBn)-Aib-Leu-Ile-AET] | | | | |
| GIP-093 | DOTA-Ttds-Asp-Trp-5Clw-Pro-Glu-leu- | 2125.036 | 2125.043 | 8.4 | A |
| | [Cys(3MeBn)-Aib-Leu-Ile-en] | | | | |
| GIP-094 | DOTA-Ttds-Asp-Trp-5Clw-Hyp-Glu-leu- | 2157.992 | 2158.000 | 8.8 | A |
| | [Cys(3MeBn)-Aib-Leu-Ile-AET] | | | | |
| GIP-095 | DOTA-Ttds-Asp-Trp-5Clw-Tap-Glu-leu- | 2157.008 | 2157.015 | 8.2 | A |
| | [Cys(3MeBn)-Aib-Leu-Ile-AET] | | | | |
| GIP-096 | DOTA-Ttds-Asp-Trp-5Clw-4Ap-Glu-leu- | 2157.008 | 2157.014 | 8.2 | A |
| | [Cys(3MeBn)-Aib-Leu-Ile-AET] | | | | |
| GIP-097 | DOTA-Ttds-Asp-Trp-5Clw-Pro-Glu-leu- | 2225.034 | 2225.037 | 9.2 | A |
| | [Cys(3MeBn)-Apc(Ac)-Leu-Ile-AET] | | | | |
| GIP-098 | DOTA-Ttds-Asp-Trp-5Clw-Pro-Glu-leu- | 2141.031 | 2141.039 | 7.2 | A |
| | [Dap(3MeBn)-Aib-Leu-Ile-AETO] | | | | |
| GIP-099 | DOTA-Ttds-Asp-Trp-5Clw-Pro-Glu-leu- | 2156.013 | 2156.020 | 9.5 | A |
| | [Cys(3MeBn)-Aib-Hle-Ile-AET] | | | | |
| GIP-100 | DOTA-Ttds-Asp-Trp-5Clw-Nmg-Glu-leu- | 2115.982 | 2115.987 | 9.1 | A |
| | [Cys(3MeBn)-Aib-Leu-Ile-AET] | | | | |
| GIP-101 | DOTA-Ttds-Asp-Trp-5Clw-Pro-Glu-leu- | 2157.992 | 2157.997 | 9.0 | A |
| | [Cys(3MeBn)-Ams-Leu-Ile-AET] | | | | |
| GIP-102 | DOTA-Ttds-Asp-Trp-5Clw-H3p-Glu-leu- | 2157.992 | 2157.989 | 8.8 | A |
| | [Cys(3MeBn)-Aib-Leu-Ile-AET] | | | | |
| GIP-103 | DOTA-APAc-Asp-Trp-5Clw-Hyp-Glu-leu- | 2038.909 | 2038.919 | 7.6 | A |
| | [Cys(2Lut)-Thp-Leu-Ile-AET] | | | | |
| GIP-104 | DOTA-Ttds-Asp-6Fw-5Clw-Pro-Glu-leu- | 2159.988 | 2159.993 | 9.2 | A |
| | [Cys(3MeBn)-Aib-Leu-Ile-AET] | | | | |
| GIP-105 | DOTA-Ttds-Asp-6Fw-5Brw-Pro-Glu-leu- | 2203.937 | 2203.941 | 9.3 | A |
| | [Cys(3MeBn)-Aib-Leu-Ile-AET] | | | | |
| GIP-106 | DOTA-APAc-Asp-Trp-5Clw-Hyp-Glu-leu- | 2021.948 | 2021.894 | 7.6 | A |
| | [Dap(2Lut)-Thp-Leu-Ile-AET] | | | | |
| GIP-107 | DOTA-APAc-Asp-Trp-5Clw-Hyp-Glu-leu- | 2020.952 | 2020.958 | 7.6 | A |
| | [Dap(3MeBn)-Thp-Leu-Ile-AET] | | | | |
| GIP-108 | HO-Succinyl-Trp-5Clw-Hyp-Glu-leu-[Cys(2Lut)- | 2357.088 | 2357.091 | 8.2 | A |
| | Thp-Leu-Ile-Cys]-Ttds-Lys(DOTA)-NH₂ | | | | |
| GIP-109 | HO-Succinyl-Trp-5Clw-Hyp-Glu-leu-[Cys(2Lut)- | 2194.999 | 2195.005 | 7.9 | A |
| | Thp-Leu-Ile-Cys]-APAc-Lys(DOTA)-NH₂ | | | | |
| GIP-110 | HO-Succinyl-Trp-5Clw-Hyp-Glu-leu-[Cys(2Lut)- | 1882.819 | 1882.827 | 8.7 | A |
| | Apc(DOTA)-Leu-Ile-AET] | | | | |
| GIP-111 | DOTA-Ttds-Asp-Trp-5Clw-4Ap(Ac)-Glu-leu- | 2199.019 | 2199.023 | 8.7 | A |
| | [Cys(3MeBn)-Aib-Leu-Ile-AET] | | | | |
| GIP-112 | DOTA-Ttds-Asp-Trp-5Clw-Tap-Glu-leu- | 2199.030 | 2199.039 | 7.2 | A |
| | [Cys(2Lut)-Apc-Leu-Ile-AET] | | | | |
| GIP-113 | DOTA-Ttds-Asp-Trp-5Clw-Tap-Glu-leu- | 2182.069 | 2182.079 | 6.8 | A |
| | [Dap(2Lut)-Apc-Leu-Ile-AET] | | | | |
| GIP-114 | HO-Succinyl-Trp-5Clw-Hyp-Glu-leu-[Cys(2Lut)- | 2194.015 | 2194.019 | 7.9 | A |
| | Apc-Leu-Ile-Cys]-APAc-Lys(DOTA)-NH₂ | | | | |
| GIP-115 | HO-Succinyl-Trp-5Clw-Hyp-Glu-leu-[Cys(2Lut)- | 2054.904 | 2054.907 | 8.2 | A |
| | Thp-Leu-Ile-Cys]-Lys(DOTA)-NH₂ | | | | |
| GIP-116 | HO-Succinyl-Trp-5Clw-Hyp-Glu-leu-[Cys(2Lut)- | 2054.904 | 2054.907 | 8.0 | A |
| | Thp-Leu-Ile-Cys]-lys(DOTA)-NH₂ | | | | |
| GIP-117 | HO-Succinyl-Trp-5Clw-Hyp-Glu-leu-[Cys(2Lut)- | 2011.898 | 2011.902 | 8.3 | A |
| | Thp-Leu-Ile-Cys]-Ape-DOTA | | | | |
| GIP-118 | HO-Succinyl-Trp-5Clw-Hyp-Glu-leu-[Cys(2Lut)- | 1995.867 | 1995.874 | 8.4 | A |
| | Thp-Leu-Ile-Cys]-PP-DOTA | | | | |
| GIP-119 | DOTA-Ttds-Asp-Trp-5Clw-Pro-Glu-leu- | 2184.019 | 2184.029 | 7.9 | A |
| | [Cys(2Lut)-Apc-Leu-Ile-AET] | | | | |
| GIP-120 | MeO2kDaPEGAc-Asp-Trp-5Clw-Hyp-Glu-leu- | n.d. | n.d. | 11.6* | A |
| | [Cys(2Lut)-Thp-Leu-Ile-Cys]-lys(DOTA)-NH₂ | | | | |
| GIP-121 | Ac-Asp-Trp-5Clw-Tap(DOTA)-Glu-leu- | 1982.846 | 1982.856 | 7.9 | A |
| | [Cys(2Lut)-Thp-Leu-Ile-Cys]-NH₂ | | | | |
| GIP-122 | Ac-Asp-Trp-5Clw-Hyp-Glu-leu-[Cys(2Lut)- | 1982.846 | 1982.867 | 8.8 | A |
| | Apc(DOTA)-Leu-Ile-Cys]-NH₂ | | | | |
| GIP-123 | Ac-Asp-Trp-5Clw-Tap-Glu-leu-[Cys(2Lut)- | 1981.862 | 1981.885 | 8.2 | A |
| | Apc(DOTA)-Leu-Ile-Cys]-NH₂ | | | | |
| GIP-124 | Ac-Asp-Trp-5Clw-Hyp-Glu-leu- | 2071.865 | 2071.894 | 9.5 | A |
| | [Cys(tMeBn(DOTA-AET))-Thp-Leu-Ile-Cys]-NH₂ | | | | |
| GIP-125 | DOTA-Asp-Trp-5Clw-Hyp-Glu-leu-[Cys(2Lut)- | 1941.820 | 1941.816 | 8.2 | A |
| | Thp-Leu-Ile-Cys]-NH₂ | | | | |
| GIP-126 | DOTA-Ttds-Tyr-Asp-Trp-Trp-Pro-Glu-Leu- | 2300.090 | 2300.097 | 9.3 | B |
| | [Cys(3MeBn)-Ala-Leu-Ile-Cys]-NH₂ | | | | |
| GIP-127 | Ac-Tyr-Asp-Trp-Trp-Pro-Glu-Leu-[Cys(3MeBn)- | 2470.195 | 2470.202 | 9.4 | B |
| | Ala-Leu-Ile-Cys]-Ttds-Lys(DOTA)-NH₂ | | | | |
| GIP-128 | DOTA-Ttds-Asp-Trp-Trp-Pro-Glu-leu- | 2151.042 | 2151.049 | 8.7 | B |
| | [Cys(3MeBn)-Aib-Leu-Ile-Cys]-NH₂ | | | | |
| GIP-129 | DOTA-Ttds-Asp-Trp-Trp-Pro-Glu-leu- | 2108.036 | 2108.044 | 8.8 | B |
| | [Cys(3MeBn)-Aib-Leu-Ile-AET] | | | | |
| GIP-130 | DOTA-Ttds-Nmd-Trp-Trp-Pro-Glu-Leu- | 2122.052 | 2122.058 | 9.2 | B |
| | [Cys(3MeBn)-Aib-Leu-Ile-AET] | | | | |
| GIP-131 | Ac-Asp-Trp-Trp-Pro-Glu-Leu-[Cys(3MeBn)-Aib- | 2321.148 | 2321.157 | 9.2 | B |
| | Leu-Ile-Cys]-Ttds-Lys(DOTA)-NH₂ | | | | |
| GIP-132 | Ac-Asp-Trp-Trp-Pro-Glu-Npg-[Cys(3MeBn)-Aib- | 2335.163 | 2335.169 | 9.5 | B |
| | Leu-Ile-Cys]-Ttds-Lys(DOTA)-NH₂ | | | | |
| GIP-133 | Ac-Asp-Trp-Trp-Pro-Glu-Leu-[Cys(3MeBn)-Aib- | 2335.163 | 2335.171 | 9.2 | B |
| | Npg-Ile-Cys]-Ttds-Lys(DOTA)-NH₂ | | | | |
| GIP-134 | Ac-Asp-Trp-Trp-Pro-Glu-Leu-[Cys(3MeBn)-Aib- | 2361.179 | 2361.188 | 9.7 | B |
| | Cha-Ile-Cys]-Ttds-Lys(DOTA)-NH₂ | | | | |
| GIP-135 | Php-Asp-Trp-Trp-Pro-Glu-leu-[Cys(3MeBn)- | 2411.195 | 2411.201 | 9.7 | B |
| | Aib-Leu-Ile-Cys]-Ttds-Lys(DOTA)-NH₂ | | | | |
| GIP-136 | 4FPhp-Asp-Trp-Trp-Pro-Glu-leu-[Cys(3MeBn)- | 2429.185 | 2429.189 | 9.8 | B |
| | Aib-Leu-Ile-Cys]-Ttds-Lys(DOTA)-NH₂ | | | | |
| GIP-137 | 40HPhp-Asp-Trp-Trp-Pro-Glu-leu- | 2427.190 | 2427.198 | 9.0 | B |
| | [Cys(3MeBn)-Aib-Leu-Ile-Cys]-Ttds-Lys(DOTA)- | | | | |
| | NH₂ | | | | |
| GIP-138 | Ac-Asp-Trp-1Ni-Pro-Glu-leu-[Cys(3MeBn)-Ala- | 2304.121 | 2304.128 | 9.0 | B |
| | Leu-Val-Cys]-Ttds-Lys(DOTA)-NH₂ | | | | |
| GIP-139 | Ac-Asp-Trp-1Ni-Pro-Glu-leu-[Cys(3MeBn)-Deg- | 2360.184 | 2360.198 | 9.3 | B |
| | Leu-Ile-Cys]-Ttds-Lys(DOTA)-NH₂ | | | | |
| GIP-140 | Ac-Asp-Trp-6Fw-Pro-Glu-leu-[Cys(3MeBn)-Aib- | 2339.138 | 2339.122 | 9.5 | B |
| | Leu-Ile-Cys]-Ttds-Lys(DOTA)-NH₂ | | | | |
| GIP-141 | DOTA-Ttds-Asp-6Clw-Trp-Pro-Glu-leu- | 2141.997 | 2142.007 | 9.2 | B |
| | [Cys(3MeBn)-Aib-Leu-Ile-AET] | | | | |
| GIP-142 | HO-Succinyl-Trp-5Clw-Hyp-Glu-leu-[Cys(2Lut)- | n.d. | n.d. | 12.3* | B |
| | Apc(MeO10kDaPEGAc)-Leu-Ile-Cys]-APAc- | | | | |
| | Lys(DOTA)-NH₂ | | | | |
| GIP-143 | HO-Succinyl-Trp-5Clw-Hyp-Glu-leu-[Cys(2Lut)- | n.d. | n.d | 11.6* | B |
| | Apc(MeO2kDaPEGAc)-Leu-Ile-Cys]-APAc- | | | | |
| | Lys(DOTA)-NH₂ | | | | |
| GIP-144 | HO-Succinyl-Trp-5Clw-Hyp-Glu-leu-[Cys(2Lut)- | n.d. | n.d. | 11.5* | B |
| | Thp-Leu-Ile-Cys]-APAc-lys(MeO2kDaPEGAc)- | | | | |
| | Lys(DOTA)-NH₂ | | | | |
| GIP-145 | Ac-Asp-Trp-5Clw-Hyp-Glu-leu- | 2080.919 | 2080.900 | 8.8 | B |
| | [Cys(tMeBn(DOTA-PP))-Thp-Leu-Ile-Cys]-NH₂ | | | | |
| GIP-146 | AGLU-Glutar-Asp-Trp-5Clw-Hyp-Glu-leu- | 2284.827 | 2284.827 | 7.7 | A |
| | [Cys(2Lut)-Apc(InDOTA)-Leu-Ile-AET] | | | | |
| GIP-147 | AGLU-Glutar-Asp-Trp-5Clw-Hyp-Glu-leu- | 2236.860 | 2236.861 | 7.9 | A |
| | [Cys(2Lut)-Apc(ZnDOTA)-Leu-Ile-AET] | | | | |
| GIP-148 | InDOTA-Ttds-Asp-Trp-5Clw-Pro-Glu-leu- | 2251.878 | 2251.886 | 9.2 | A |
| | [Cys(3MeBn)-Aib-Leu-Ile-AET] | | | | |
| GIP-149 | InDOTA-Ttds-Asp-Trp-5Clw-Pro-Glu-leu- | 2267.873 | 2267.879 | 9.0 | A |
| | [Cys(3MeBn)-Ams-Leu-Ile-AET] | | | | |
| GIP-150 | InDOTA-Ttds-Asp-Trp-5Clw-Pro-Glu-leu- | 2293.888 | 2293.894 | 9.3 | A |
| | [Cys(3MeBn)-Thp-Leu-Ile-AET] | | | | |
| GIP-151 | InDOTA-Ttds-Asp-Trp-5Clw-Hyp-Glu-leu- | 2267.873 | 2267.880 | 8.9 | A |
| | [Cys(3MeBn)-Aib-Leu-Ile-AET] | | | | |
| GIP-152 | InDOTA-Ttds-Asp-Trp-5Clw-Nmg-Glu-leu- | 2225.862 | 2225.869 | 9.0 | A |
| | [Cys(3MeBn)-Aib-Leu-Ile-AET] | | | | |
| GIP-153 | InDOTA-APAc-Asp-Trp-5Clw-Pro-Glu-leu- | 2089.789 | 2089.794 | 8.8 | A |
| | [Cys(3MeBn)-Aib-Leu-Ile-AET] | | | | |
| GIP-154 | InDOTA-Ttds-Asp-Trp-5Clw-Pro-Glu-leu- | 2252.873 | 2252.880 | 8.0 | A |
| | [Cys(2Lut)-Aib-Leu-Ile-AET] | | | | |
| GIP-155 | InDOTA-APAc-Asp-Trp-5Clw-Hyp-Glu-leu- | 2148.789 | 2148.796 | 7.7 | A |
| | [Cys(2Lut)-Thp-Leu-Ile-AET] | | | | |
| GIP-156 | Ac-Asp-Trp-5Clw-Pro-Glu-leu-[Cys(3MeBn)- | 2527.026 | 2527.028 | 9.1 | A |
| | Aib-Leu-Ile-Cys]-Ttds-Lys(LuDOTA)-NH₂ | | | | |
| GIP-157 | LuDOTA-Ttds-Asp-Trp-5Clw-Pro-Glu-leu- | 2329.910 | 2329.914 | 9.0 | A |
| | [Cys(3MeBn)-Ams-Leu-Ile-AET] | | | | |
| GIP-158 | LuDOTA-Ttds-Asp-Trp-5Clw-Pro-Glu-leu- | 2355.925 | 2355.927 | 9.4 | A |
| | [Cys(3MeBn)-Thp-Leu-Ile-AET] | | | | |
| GIP-159 | LuDOTA-Ttds-Asp-Trp-5Clw-Hyp-Glu-leu- | 2329.910 | 2329.912 | 8.9 | A |
| | [Cys(3MeBn)-Aib-Leu-Ile-AET] | | | | |
| GIP-160 | LuDOTA-Ttds-Asp-Trp-5Clw-Nmg-Glu-leu- | 2287.899 | 2287.903 | 9.0 | A |
| | [Cys(3MeBn)-Aib-Leu-Ile-AET] | | | | |
| GIP-161 | LuDOTA-APAc-Asp-Trp-5Clw-Pro-Glu-leu- | 2151.825 | 2151.829 | 8.9 | A |
| | [Cys(3MeBn)-Aib-Leu-Ile-AET] | | | | |
| GIP-162 | LuDOTA-APAc-Asp-Trp-5Clw-Hyp-Glu-leu- | 2210.826 | 2210.827 | 7.7 | A |
| | [Cys(2Lut)-Thp-Leu-Ile-AET] | | | | |
| GIP-163 | LuDOTA-Ttds-Asp-Trp-5Clw-Pro-Glu-leu- | 2314.910 | 2314.912 | 8.1 | A |
| | [Cys(2Lut)-Aib-Leu-Ile-AET] | | | | |
| GIP-164 | Ac-Asp-Trp-5Clw-Pro-Glu-leu-[Cys(3MeBn)- | 2464.989 | 2464.997 | 9.1 | A |
| | Aib-Leu-Ile-Cys]-Ttds-Lys(InDOTA)-NH₂ | | | | |
| GIP-165 | Ac-Asp-Trp-1Ni-Pro-Glu-leu-[Cys(3MeBn)-Aib- | 2442.033 | 2442.038 | 9.3 | A |
| | Leu-Ile-Cys]-Ttds-Lys(InDOTA)-NH₂ | | | | |
| GIP-166 | Ac-Asp-Trp-1Ni-Pro-Glu-leu-[Cys(3MeBn)-Egz- | 2482.064 | 2482.067 | 9.6 | A |
| | Leu-Ile-Cys]-Ttds-Lys(InDOTA)-NH₂ | | | | |
| GIP-167 | LuDOTA-Ttds-Asp-Trp-5Clw-Pro-Glu-leu- | 2313.915 | 2313.919 | 9.2 | A |
| | [Cys(3MeBn)-Aib-Leu-Ile-AET] | | | | |
| GIP-168 | HO-Succinyl-Trp-5Clw-Hyp-Glu-leu-[Cys(2Lut)- | 2304.879 | 2304.882 | 8.0 | A |
| | Thp-Leu-Ile-Cys]-APAc-Lys(InDOTA)-NH₂ | | | | |
| GIP-169 | HO-Succinyl-Trp-5Clw-Hyp-Glu-leu-[Cys(2Lut)- | 1992.700 | 1992.701 | 8.7 | A |
| | Apc(InDOTA)-Leu-Ile-AET] | | | | |
| GIP-170 | HO-Succinyl-Trp-5Clw-Hyp-Glu-leu-[Cys(2Lut)- | 2366.916 | 2366.920 | 8.1 | A |
| | Thp-Leu-Ile-Cys]-APAc-Lys(LuDOTA)-NH₂ | | | | |
| GIP-171 | HO-Succinyl-Trp-5Clw-Hyp-Glu-leu-[Cys(2Lut)- | 2054.736 | 2054.738 | 8.7 | A |
| | Apc(LuDOTA)-Leu-Ile-AET] | | | | |
| GIP-172 | Ac-Asp-Trp-1Ni-Pro-Glu-leu-[Cys(3MeBn)-Aib- | 2504.070 | 2504.072 | 9.3 | B |
| | Leu-Ile-Cys]-Ttds-Lys(LuDOTA)-NH₂ | | | | |
| GIP-173 | Ac-Asp-Trp-1Ni-Pro-Glu-leu-[Cys(3MeBn)-Egz- | 2544.101 | 2544.104 | 9.7 | B |
| | Leu-Ile-Cys]-Ttds-Lys(LuDOTA)-NH₂ | | | | |
| GIP-174 | AGLU-Glutar-Asp-Trp-5Clw-Hyp-Glu-leu- | 2264.978 | 2264.987 | 7.4 | A |
| | [Cys(2Lut)-Thp-Leu-Ile-Cys]-APAc-glu(AGLU)- | | | | |
| | NH₂ | | | | |
| GIP-175 | Ac-Asp-Trp-5Clw-Tap(AGLU-Glutar)-Glu-leu- | 1873.782 | 1873.784 | 8.3 | A |
| | [Cys(2Lut)-Thp-Leu-Ile-Cys]-NH₂ | | | | |
| GIP-176 | Ac-Asp-Trp-5Clw-Nglu(AGLU)-Glu-leu- | 1776.729 | 1776.731 | 8.4 | A |
| | [Cys(2Lut)-Thp-Leu-Ile-Cys]-NH₂ | | | | |
| GIP-177 | Ac-Asp-Trp-5Clw-Hyp-Glu-leu-[Cys(2Lut)-Thp- | 2029.872 | 2029.882 | 8.1 | A |
| | Leu-Ile-Cys]-APAc-glu(AGLU)-NH₂ | | | | |
| GIP-178 | AGLU-Glutar-Asp-Trp-5Clw-Hyp-Glu-leu- | 1832.756 | 1832.770 | 8.3 | A |
| | [Cys(2Lut)-Thp-Leu-Ile-Cys]-NH₂ | | | | |
| GIP-179 | Ac-Cya-Trp-5Clw-Hyp-Glu-leu-[Cys(2Lut)-Aib- | 1548.601 | 1548.603 | 8.7 | A |
| | Leu-Ile-AET] | | | | |
| GIP-180 | Ac-Asp-Trp-5Clw-Hyp-Glu-leu-[Cys(2Lut)-Thp- | 1597.650 | 1597.659 | 9.0 | A |
| | Leu-Ile-Cys]-NH₂ | | | | |
| GIP-181 | Ac-Asp-Trp-5Clw-Hyp-Glu-leu-[Cys(2Lut)-Thp- | 1598.634 | 1598.643 | 8.8 | A |
| | Leu-Ile-Cys]-OH | | | | |
| GIP-182 | Ac-Asp-Trp-5Clw-Hyp-Glu-leu-[Cys(2Lut)-Thp- | 1584.655 | 1584.662 | 8.9 | A |
| | Leu-Ile-Cysol]-OH | | | | |
| GIP-183 | Ac-Asp-Trp-5Clw-Nmg-Glu-leu-[Cys(2Lut)-Thp- | 1555.640 | 1555.641 | 8.8 | A |
| | Leu-Ile-Cys]-NH₂ | | | | |
| GIP-184 | Ac-Asp-Trp-5Clw-Nleu-Glu-leu-[Cys(2Lut)-Thp- | 1597.687 | 1597.690 | 9.6 | A |
| | Leu-Ile-Cys]-NH₂ | | | | |
| GIP-185 | Ac-Asp-Trp-5Clw-Nphe-Glu-leu-[Cys(2Lut)- | 1631.671 | 1631.674 | 9.7 | A |
| | Thp-Leu-Ile-Cys]-NH₂ | | | | |
| GIP-186 | Ac-Asp-Trp-5Clw-Nlys-Glu-leu-[Cys(2Lut)-Thp- | 1612.697 | 1612.701 | 8.1 | A |
| | Leu-Ile-Cys]-NH₂ | | | | |
| GIP-187 | Ac-Asp-Trp-5Clw-4Tfp-Glu-leu-[Cys(2Lut)-Thp- | 1599.646 | 1599.659 | 9.4 | A |
| | Leu-Ile-Cys]-NH₂ | | | | |
| GIP-188 | Ac-Ser-Trp-5Clw-Hyp-Glu-leu-[Cys(2Lut)-Thp- | 1569.655 | 1569.666 | 8.9 | A |
| | Leu-Ile-Cys]-NH₂ | | | | |
| GIP-189 | Ac-Cya-Trp-5Clw-Hyp-Glu-leu-[Cys(2Lut)-Thp- | 1633.617 | 1633.627 | 8.7 | A |
| | Leu-Ile-Cys]-NH₂ | | | | |
| GIP-190 | Ac-Asn-Trp-5Clw-Hyp-Glu-leu-[Cys(2Lut)-Thp- | 1596.666 | 1596.677 | 8.9 | A |
| | Leu-Ile-Cys]-NH₂ | | | | |
| GIP-191 | Ac-Glu-Trp-5Clw-Hyp-Glu-leu-[Cys(2Lut)-Thp- | 1611.666 | 1611.677 | 8.9 | A |
| | Leu-Ile-Cys]-NH₂ | | | | |
| GIP-192 | Ac-Asp-Trp-5Clw-Hyp-Asp-leu-[Cys(2Lut)-Thp- | 1583.634 | 1583.646 | 8.8 | A |
| | Leu-Ile-Cys]-NH₂ | | | | |
| GIP-193 | Ac-Asp-Trp-5Clw-Hyp-Gln-leu-[Cys(2Lut)-Thp- | 1596.666 | 1596.679 | 8.7 | A |
| | Leu-Ile-Cys]-NH₂ | | | | |
| GIP-194 | Ac-Asp-Trp-5Clw-Hyp-Hse-leu-[Cys(2Lut)-Thp- | 1569.655 | 1569.667 | 8.9 | A |
| | Leu-Ile-Cys]-NH₂ | | | | |
| GIP-195 | Ac-Hse-Trp-5Clw-Hyp-Glu-leu-[Cys(2Lut)-Thp- | 1583.671 | 1583.686 | 8.9 | A |
| | Leu-Ile-Cys]-NH₂ | | | | |
| GIP-196 | Ac-Asp-Trp-5Clw-Nglu-Glu-leu-[Cys(2Lut)-Thp- | 1613.645 | 1613.648 | 9.0 | A |
| | Leu-Ile-Cys]-NH₂ | | | | |
| GIP-197 | Ac-Hse-Trp-5Brw-Hyp-Glu-leu-[Cys(2Lut)-Thp- | 1627.620 | 1627.637 | 9.4 | A |
| | Leu-Ile-Cys]-NH₂ | | | | |
| GIP-198 | HO-Succinyl-Trp-5Clw-Pro-Glu-leu- | 1481.628 | 1481.631 | 11.3 | A |
| | [Cys(3MeBn)-Aib-Leu-Ile-Cys]-NH₂ | | | | |
| GIP-199 | HO-Succinyl-Trp-5Clw-Pro-Glu-leu- | 1523.638 | 1523.642 | 11.5 | A |
| | [Cys(3MeBn)-Thp-Leu-Ile-Cys]-NH₂ | | | | |
| GIP-200 | Ac-Asp-Trp-5Clw-Hyp-Glu-leu-[Hcy(2Lut)-Thp- | 1611.666 | 1611.680 | 9.5 | A |
| | Leu-Ile-Cys]-NH₂ | | | | |
| GIP-201 | Ac-Asp-Trp-5Clw-Hyp-Glu-leu-[Cys(2Lut)-Thp- | 1611.666 | 1611.684 | 9.6 | A |
| | Leu-Ile-Hcy]-NH₂ | | | | |
| GIP-202 | Ac-Asp-Trp-5Clw-Hyp-Glu-leu-[Hcy(2Lut)-Thp- | 1625.681 | 1625.697 | 9.7 | A |
| | Leu-Ile-Hcy]-NH₂ | | | | |
| GIP-203 | Ac-Asp-Trp-5Clw-Hyp-Glu-leu-[Cys(2Lut)-Eca- | 1581.655 | 1581.672 | 9.5 | A |
| | Leu-Ile-Cys]-NH₂ | | | | |
| GIP-204 | Ac-Gln-Trp-5Clw-Hyp-Glu-leu-[Cys(2Lut)-Thp- | 1610.682 | 1610.700 | 9.2 | A |
| | Leu-Ile-Cys]-NH₂ | | | | |
| GIP-205 | Ac-Asp-Trp-5Clw-Hyp-Asn-leu-[Cys(2Lut)-Thp- | 1582.650 | 1582.669 | 8.9 | A |
| | Leu-Ile-Cys]-NH₂ | | | | |
| GIP-206 | Ac-Asp-Trp-5Clw-Hyp-Glu-leu-[Cys(2Lut)-Aic- | 1629.655 | 1629.675 | 9.9 | A |
| | Leu-Ile-Cys]-NH₂ | | | | |
| GIP-207 | Ac-Pse-Trp-5Clw-Hyp-Glu-leu-[Cys(2Lut)-Thp- | 1649.622 | 1649.641 | 9.1 | A |
| | Leu-Ile-Cys]-NH₂ | | | | |
| GIP-208 | Ac-Asp-Trp-5Clw-Hyp-Pse-leu-[Cys(2Lut)-Thp- | 1635.606 | 1635.620 | 8.3 | A |
| | Leu-Ile-Cys]-NH₂ | | | | |
| GIP-209 | Ac-Asp-Mcf-5Clw-Hyp-Glu-leu-[Cys(2Lut)-Thp- | 1592.600 | 1592.611 | 10.0 | A |
| | Leu-Ile-Cys]-NH₂ | | | | |
| GIP-210 | Ac-Asp-Pcf-5Clw-Hyp-Glu-leu-[Cys(2Lut)-Thp- | 1592.600 | 1592.609 | 10.0 | A |
| | Leu-Ile-Cys]-NH₂ | | | | |
| GIP-211 | Ac-Asp-Eaa-5Clw-Hyp-Glu-leu-[Cys(2Lut)-Thp- | 1626.561 | 1626.569 | 10.4 | A |
| | Leu-Ile-Cys]-NH₂ | | | | |
| GIP-212 | Ac-Asp-Egm-5Clw-Hyp-Glu-leu-[Cys(2Lut)-Thp- | 1626.561 | 1626.571 | 10.5 | A |
| | Leu-Ile-Cys]-NH₂ | | | | |
| GIP-213 | Ac-Asp-Trp-Eaa-Hyp-Glu-leu-[Cys(2Lut)-Thp- | 1592.600 | 1592.613 | 9.9 | A |
| | Leu-Ile-Cys]-NH₂ | | | | |
| GIP-214 | HO-Glutar-Trp-5Clw-Hyp-Glu-leu-[Cys(2Lut)- | 1554.644 | 1554.657 | 9.5 | A |
| | Thp-Leu-Ile-Cys]-NH₂ | | | | |
| GIP-215 | Ac-Asp-Trp-5Clw-Dtc-Glu-leu-[Cys(2Lut)-Thp- | 1627.643 | 1627.654 | 10.2 | A |
| | Leu-Ile-Cys]-NH₂ | | | | |
| GIP-216 | Ac-Asp-Trp-5Clw-Oxa-Glu-leu-[Cys(2Lut)-Thp- | 1583.634 | 1583.639 | 9.6 | A |
| | Leu-Ile-Cys]-NH₂ | | | | |
| GIP-217 | Ac-Asp-Trp-5Clw-Hyp-Nme-Leu-[Cys(2Lut)- | 1611.666 | 1611.672 | 9.4 | A |
| | Thp-Leu-Ile-Cys]-NH₂ | | | | |
| GIP-218 | Ac-Asp-Trp-5Clw-Hyp-Glu-Ile-[Cys(2Lut)-Thp- | 1597.650 | 1597.656 | 10.2 | A |
| | Leu-Ile-Cys]-NH₂ | | | | |
| GIP-219 | Ac-Asp-Trp-5Clw-Hyp-Glu-Val-[Cys(2Lut)-Thp- | 1583.634 | 1583.639 | 9.9 | A |
| | Leu-Ile-Cys]-NH₂ | | | | |
| GIP-220 | Ac-Asp-Hyw-5Clw-Hyp-Glu-leu-[Cys(2Lut)-Thp- | 1613.645 | 1613.668 | 8.7 | A |
| | Leu-Ile-Cys]-NH₂ | | | | |
| GIP-221 | Ac-Asp-7Fw-5Clw-Hyp-Glu-leu-[Cys(2Lut)-Thp- | 1615.641 | 1615.661 | 9.7 | A |
| | Leu-Ile-Cys]-NH₂ | | | | |
| GIP-222 | Ac-Asp-Trp-5Clw-Eaz-Glu-leu-[Cys(2Lut)-Thp- | 1599.612 | 1599.619 | 9.8 | A |
| | Leu-Ile-Cys]-NH₂ | | | | |
| GIP-223 | Ac-Asp-Trp-5Clw-Hyp-glu-Leu-[Cys(2Lut)-Thp- | 1597.650 | 1597.664 | 9.8 | A |
| | Leu-Ile-Cys]-NH₂ | | | | |
| GIP-224 | Ac-Asp-Phe-5Clw-Hyp-Glu-leu-[Cys(2Lut)-Thp- | 1558.639 | 1558.647 | 9.5 | A |
| | Leu-Ile-Cys]-NH₂ | | | | |
| GIP-226 | Ac-Asp-Egp-5Clw-Hyp-Glu-leu-[Cys(2Lut)-Thp- | 1626.561 | 1626.575 | 10.3 | A |
| | Leu-Ile-Cys]-NH₂ | | | | |
| GIP-227 | Ac-Asp-Trp-7Fw-Hyp-Glu-leu-[Cys(2Lut)-Thp- | 1581.680 | 1581.692 | 9.2 | A |
| | Leu-Ile-Cys]-NH₂ | | | | |
| GIP-228 | Ac-Asp-Trp-5Clw-Hyp-Glu-leu-[Cys(2Lut)-Phe- | 1617.655 | 1617.652 | 9.6 | A |
| | Leu-Ile-Cys]-NH₂ | | | | |
| GIP-229 | Ac-Asp-Trp-5Clw-Hyp-Glu-leu-[Cys(2Lut)-Thp- | 1597.650 | 1597.653 | 9.5 | A |
| | Ile-Ile-Cys]-NH₂ | | | | |
| GIP-230 | Ac-Asp-Trp-5Clw-Hyp-Glu-leu-[Cys(2Lut)-Thp- | 1640.667 | 1640.675 | 7.7 | A |
| | Arg-Ile-Cys]-NH₂ | | | | |
| GIP-231 | Ac-Asp-Trp-5Clw-Hyp-Glu-leu-[Cys(2Lut)-Thp- | 1613.609 | 1613.622 | 8.1 | A |
| | Glu-Ile-Cys]-NH₂ | | | | |
| GIP-232 | Ac-Asp-Trp-5Clw-Hyp-Glu-leu-[Cys(2Lut)-Thp- | 1631.634 | 1631.649 | 9.3 | A |
| | Phe-Ile-Cys]-NH₂ | | | | |
| GIP-233 | Ac-Asp-Trp-5Clw-Hyp-Glu-leu-[Cys(2Lut)-Thp- | 1670.645 | 1670.675 | 9.3 | A |
| | Trp-Ile-Cys]-NH₂ | | | | |
| GIP-234 | Ac-Asp-Trp-5Clw-Hyp-Glu-leu-[Cys(2Lut)-Trp- | 1656.666 | 1656.700 | 9.5 | A |
| | Leu-Ile-Cys]-NH₂ | | | | |
| GIP-235 | Ac-Asp-Trp-5Clw-Hyp-Glu-leu-[Cys(2Lut)-Glu- | 1599.629 | 1599.628 | 9.1 | A |
| | Leu-Ile-Cys]-NH₂ | | | | |
| GIP-236 | Ac-Asp-Trp-5Clw-Hyp-Glu-leu-[Cys(2Lut)-Thp- | 1571.598 | 1571.623 | 8.0 | A |
| | Ser-Ile-Cys]-NH₂ | | | | |
| GIP-237 | Ac-Asp-Trp-5Clw-Hyp-Glu-leu-[Cys(2Lut)-Thp- | 1597.650 | 1597.659 | 9.5 | A |
| | Leu-Leu-Cys]-NH₂ | | | | |
| GIP-238 | Ac-Asp-Trp-5Clw-Hyp-Glu-leu-[Cys(2Lut)-Thp- | 1597.650 | 1597.697 | 9.5 | A |
| | Leu-Tle-Cys]-NH₂ | | | | |
| GIP-239 | Ac-Asp-Trp-5Clw-Tap-Glu-leu-[Cys(2Lut)-Thp- | 1596.666 | 1596.638 | 8.5 | A |
| | Leu-Ile-Cys]-NH₂ | | | | |
| GIP-240 | Ac-Tyr-Asp-Trp-Trp-Pro-Glu-Leu-[Cys(3MeBn)- | 1653.736 | 1653.738 | 10.7 | B |
| | Ala-Leu-Ile-Cys]-NH₂ | | | | |
| GIP-241 | H-Met-Tyr-Asp-Trp-Trp-Pro-Glu-Leu- | 1799.787 | 1799.790 | 9.7 | B |
| | [Cys(3MeBn)-Gln-Leu-Ile-Cys]-NH₂ | | | | |
| GIP-242 | H-Met-Tyr-Asp-Trp-Trp-Pro-Glu-Leu- | 1772.777 | 1772.780 | 9.7 | B |
| | [Cys(3MeBn)-Thr-Leu-Ile-Cys]-NH₂ | | | | |
| GIP-243 | H-Met-Tyr-Asp-Trp-Trp-Pro-Glu-Leu- | 1799.824 | 1799.829 | 9.3 | B |
| | [Cys(3MeBn)-Lys-Leu-Ile-Cys]-NH₂ | | | | |
| GIP-244 | Ac-Asp-Trp-Trp-Pro-Glu-Leu-[Cys(3MeBn)-Ala- | 1490.673 | 1490.676 | 10.6 | B |
| | Leu-Ile-Cys]-NH₂ | | | | |
| GIP-245 | H-Met-Tyr-Asp-Trp-Trp-Pro-glu-Leu- | 1742.766 | 1742.790 | 9.5 | B |
| | [Cys(3MeBn)-Ala-Leu-Ile-Cys]-NH₂ | | | | |
| GIP-246 | H-Met-tyr-Asp-Trp-Trp-Pro-Glu-Leu- | 1742.766 | 1742.769 | 9.8 | B |
| | [Cys(3MeBn)-Ala-Leu-Ile-Cys]-NH₂ | | | | |
| GIP-247 | H-Met-Tyr-Asp-Trp-Trp-Pro-Glu-leu- | 1742.766 | 1742.769 | 9.5 | B |
| | [Cys(3MeBn)-Ala-Leu-Ile-Cys]-NH₂ | | | | |
| GIP-248 | H-Met-Tyr-Asp-Trp-Trp-Pro-Glu-Leu- | 1742.766 | 1742.765 | 9.5 | B |
| | [Cys(3MeBn)-ala-Leu-Ile-Cys]-NH₂ | | | | |
| GIP-249 | H-Met-Ala-Asp-Trp-Trp-Pro-Glu-Leu- | 1650.740 | 1650.743 | 9.6 | B |
| | [Cys(3MeBn)-Ala-Leu-Ile-Cys]-NH₂ | | | | |
| GIP-250 | Ac-Asp-Trp-Trp-Pro-Glu-Leu-[Cys(3MeBn)-ala- | 1447.667 | 1447.670 | 10.4 | B |
| | Leu-Ile-AET] | | | | |
| GIP-251 | Ac-Asp-Trp-Trp-Pro-Glu-Leu-[Cys(3MeBn)-Aib- | 1461.683 | 1461.685 | 10.9 | B |
| | Leu-Ile-AET] | | | | |
| GIP-252 | Ac-Asp-Trp-1Ni-Oic-Glu-Leu-[Cys(3MeBn)-Aib- | 1569.740 | 1569.742 | 11.8 | B |
| | Leu-Ile-Cys]-NH₂ | | | | |
| GIP-253 | Ac-Asp-Trp-1Ni-Pip-Glu-Leu-[Cys(3MeBn)-Aib- | 1529.709 | 1529.711 | 11.6 | B |
| | Leu-Ile-Cys]-NH₂ | | | | |
| GIP-254 | H-Met-Tyr-Asp-Trp-Trp-Pro-Glu-Leu- | 1742.766 | 1742.776 | 9.8 | B |
| | [Cys(3MeBn)-Ala-Leu-Ile-Cys]-NH₂ | | | | |
| GIP-255 | HO-Succinyl-Trp-Trp-Pro-Glu-Leu- | 1433.651 | 1433.667 | 11.5 | B |
| | [Cys(3MeBn)-Ala-Leu-Ile-Cys]-NH₂ | | | | |
| GIP-256 | HO-Succinyl-Trp-Trp-Pro-Glu-Leu- | 1390.645 | 1390.659 | 11.6 | B |
| | [Cys(3MeBn)-Ala-Leu-Ile-AET] | | | | |
| GIP-257 | HO-Succinyl-Trp-Trp-Pro-Glu-Leu-[Cys(2Lut)- | 1434.647 | 1434.653 | 9.8 | B |
| | Ala-Leu-Ile-Cys]-NH₂ | | | | |
| GIP-258 | HO-Succinyl-Trp-Trp-Pro-Glu-leu- | 1447.667 | 1447.672 | 10.9 | B |
| | [Cys(3MeBn)-Aib-Leu-Ile-Cys]-NH₂ | | | | |
| GIP-259 | HO-Succinyl-Trp-Trp-Pro-Glu-Leu- | 1447.667 | 1447.674 | 11.6 | B |
| | [Cys(3MeBn)-Aib-Leu-Ile-Cys]-NH₂ | | | | |
| GIP-260 | HO-Succinyl-Trp-Trp-Pro-Glu-Leu- | 1461.683 | 1461.686 | 12.1 | B |
| | [Cys(3MeBn)-Aib-Hle-Ile-Cys]-NH₂ | | | | |
| GIP-261 | HO-Succinyl-Trp-Trp-Pro-Glu-Leu- | 1489.677 | 1489.683 | 11.6 | B |
| | [Cys(3MeBn)-Thp-Leu-Ile-Cys]-NH₂ | | | | |
| GIP-262 | Ac-Asp-Ocf-5Clw-Hyp-Glu-leu-[Cys(2Lut)-Thp- | 1592.600 | 1592.615 | 9.9 | B |
| | Leu-Ile-Cys]-NH₂ | | | | |
| GIP-263 | Ac-Asp-Egn-5Clw-Hyp-Glu-leu-[Cys(2Lut)-Thp- | 1626.561 | 1626.569 | 10.5 | B |
| | Leu-Ile-Cys]-NH₂ | | | | |
| GIP-264 | Ac-Asp-Trp-Egn-Hyp-Glu-leu-[Cys(2Lut)-Thp- | 1592.600 | 1592.606 | 9.9 | B |
| (SEQ ID | Leu-Ile-Cys]-NH₂ | | | | |
| NO: 316 | | | | | |
| GIP-265 | Ac-Asp-Trp-Egp-Hyp-Glu-leu-[Cys(2Lut)-Thp- | 1592.600 | 1592.612 | 9.6 | B |
| | Leu-Ile-Cys]-NH₂ | | | | |
| GIP-266 | Ac-Asp-Trp-5Clw-Hyp-Glu-leu-[Cys(2Lut)-Thp- | 1631.634 | 1631.674 | 9.5 | B |
| | Leu-Phe-Cys]-NH₂ | | | | |
| GIP-267 | Ac-Asp-Trp-7Nw-Hyp-Glu-leu-[Cys(2Lut)-Thp- | 1564.684 | 1564.729 | 7.4 | B |
| | Leu-Ile-Cys]-NH₂ | | | | |
| GIP-268 | DOTA-Ttds-Asp-Trp-Trp-Pro-Glu-leu- | 2137.026 | 2137.035 | 8.3 | C |
| (SEQ ID | [Cys(3MeBn)-ala-Leu-Ile-Cys]-NH₂ | | | | |
| NO: 317) | | | | | |
| GIP-269 | DOTA-Ttds-Asp-Trp-Trp-Pro-glu-Leu- | 2137.026 | 2137.034 | 8.5 | C |
| (SEQ ID | [Cys(3MeBn)-ala-Leu-Ile-Cys]-NH₂ | | | | |
| NO: 318) | | | | | |
| GIP-270 | DOTA-Ttds-Asp-Trp-Trp-Pro-glu-Leu- | 2151.042 | 2151.050 | 8.8 | C |
| | [Cys(3MeBn)-Aib-Leu-Ile-Cys]-NH₂ | | | | |
| GIP-271 | DOTA-Ttds-asp-Trp-Trp-Pro-Glu-Leu- | 2137.026 | 2137.034 | 8.8 | C |
| (SEQ ID | [Cys(3MeBn)-ala-Leu-Ile-Cys]-NH₂ | | | | |
| NO: 319) | | | | | |
| GIP-272 | Ac-Asp-2Ni-Trp-Pro-Glu-Leu-[Cys(3MeBn)-Aib- | 2332.152 | 2332.160 | 9.8 | C |
| | Leu-Ile-Cys]-Ttds-Lys(DOTA)-NH₂ | | | | |
| GIP-273 | Ac-Asp-Bta-Trp-Pro-Glu-Leu-[Cys(3MeBn)-Aib- | 2338.109 | 2338.121 | 9.7 | C |
| | Leu-lle-Cys]-Ttds-Lys(DOTA)-NH₂ | | | | |
| GIP-274 | Ac-Asp-Trp-Trp-Pro-Nme-Leu-[Cys(3MeBn)- | 2335.163 | 2335.168 | 8.8 | C |
| | Aib-Leu-Ile-Cys]-Ttds-Lys(DOTA)-NH₂ | | | | |
| GIP-275 | Ac-Asp-Trp-Trp-Pro-Glu-Tle-[Cys(3MeBn)-Aib- | 2321.148 | 2321.153 | 9.3 | C |
| | Leu-Ile-Cys]-Ttds-Lys(DOTA)-NH₂ | | | | |
| GIP-276 | Ac-Asp-Trp-Trp-Pro-Glu-Leu-[Cys(3MeBn)-Aib- | 2321.148 | 2321.153 | 9.2 | C |
| | Tle-Ile-Cys]-Ttds-Lys(DOTA)-NH₂ | | | | |
| GIP-277 | Ac-Asp-5Clw-Trp-Pro-Glu-Leu-[Cys(3MeBn)- | 2355.109 | 2355.116 | 9.5 | C |
| | Aib-Leu-Ile-Cys]-Ttds-Lys(DOTA)-NH₂ | | | | |
| GIP-278 | Ac-Asp-Trp-Trp-Pro-Glu-Leu-[Cys(3MeBn)-Aib- | 2361.179 | 2361.187 | 9.9 | C |
| (SEQ ID | Leu-Cha-Cys]-Ttds-Lys(DOTA)-NH₂ | | | | |
| NO: 320 | | | | | |
| GIP-279 | 4ClPhp-Asp-Trp-Trp-Pro-Glu-leu-[Cys(3MeBn)- | 2445.156 | 2445.159 | 10.2 | C |
| | Aib-Leu-Ile-Cys]-Ttds-Lys(DOTA)-NH₂ | | | | |
| GIP-280 | Nph1p-Asp-Trp-Trp-Pro-Glu-leu-[Cys(3MeBn)- | 2461.210 | 2461.215 | 10.2 | C |
| (SEQ ID | Aib-Leu-Ile-Cys]-Ttds-Lys(DOTA)-NH₂ | | | | |
| NO: 321) | | | | | |
| GIP-281 | Nph2p-Asp-Trp-Trp-Pro-Glu-leu-[Cys(3MeBn)- | 2461.210 | 2461.215 | 10.3 | C |
| | Aib-Leu-Ile-Cys]-Ttds-Lys(DOTA)-NH₂ | | | | |
| GIP-282 | Ac-Asp-Trp-6Clw-Pro-Glu-leu-[Cys(3MeBn)- | 2355.109 | 2355.114 | 9.1 | C |
| | Aib-Leu-Ile-Cys]-Ttds-Lys(DOTA)-NH₂ | | | | |
| GIP-283 | DOTA-Ttds-Asp-6Fw-Trp-Pro-Glu-leu- | 2126.027 | 2126.033 | 8.9 | C |
| | [Cys(3MeBn)-Aib-Leu-Ile-AET] | | | | |
| GIP-284 | DOTA-APAc-Asp-Gly-5Clw-Hyp-Glu-leu- | 1909.851 | 1909.853 | 7.3 | C |
| | [Cys(2Lut)-Thp-Leu-Ile-AET] | | | | |
| GIP-285 | DOTA-Ttds-Asp-Trp-Trp-Pro-Glu-leu- | 2138.010 | 2138.015 | 8.3 | D |
| (SEQ ID | [Cys(3MeBn)-ala-Leu-Ile-Cys]-OH | | | | |
| NO: 322 | | | | | |
| GIP-286 | HO-Succinyl-Trp-Trp-Pro-Glu-leu- | 1917.916 | 1917.917 | 7.3 | D |
| (SEQ ID | [Cys(tMeBn(DOTA-PP))-ala-Leu-Ile-Cys]-NH₂ | | | | |
| NO: 323) | | | | | |
| GIP-287 | HO-Succinyl-Trp-Trp-Pro-Glu-leu- | 1908.861 | 1908.867 | 8.0 | D |
| (SEQ ID | [Cys(tMeBn(DOTA-AET))-ala-Leu-Ile-Cys]-NH₂ | | | | |
| NO: 324) | | | | | |
| GIP-288 | HO-Succinyl-Trp-Trp-Pro-Glu-leu- | 2250.111 | 2250.115 | 8.5 | D |
| (SEQ ID | [Cys(3MeBn)-ala-Leu-Ile-Cys]-Ttds-Lys(DOTA)- | | | | |
| NO: 325) | NH₂ | | | | |
| GIP-289 | Ac-Asp-Trp-Nmw-Pro-Glu-Leu-[Cys(3MeBn)- | 2335.163 | 2335.172 | 8.8 | D |
| (SEQ ID | Aib-Leu-Ile-Cys]-Ttds-Lys(DOTA)-NH₂ | | | | |
| NO: 326) | | | | | |
| GIP-290 | Ac-Asp-Trp-Trp-Pro-Glu-Nml-[Cys(3MeBn)- | 2335.163 | 2335.175 | 8.5 | D |
| (SEQ ID | Aib-Leu-Ile-Cys]-Ttds-Lys(DOTA)-NH₂ | | | | |
| NO: 327) | | | | | |
| GIP-291 | Ac-Asp-Trp-Trp-Pro-Glu-Leu-[Nmc(3MeBn)- | 2335.163 | 2335.170 | 8.8 | D |
| (SEQ ID | Aib-Leu-Ile-Cys]-Ttds-Lys(DOTA)-NH₂ | | | | |
| NO: 328 | | | | | |
| GIP-292 | Ac-Asp-Trp-Trp-Pro-Glu-Leu-[Cys(3MeBn)-Aib- | 2335.163 | 2335.169 | 8.7 | D |
| (SEQ ID | Leu-Ile-Nmc]-Ttds-Lys(DOTA)-NH₂ | | | | |
| NO: 329) | | | | | |
| GIP-293 | Ac-Asp-1Ni-Trp-Pro-Glu-Leu-[Cys(3MeBn)-Aib- | 2332.152 | 2332.162 | 9.8 | D |
| (SEQ ID | Leu-Ile-Cys]-Ttds-Lys(DOTA)-NH₂ | | | | |
| NO: 330) | | | | | |
| GIP-294 | Ac-Asp-Trp-2Ni-Pro-Glu-Leu-[Cys(3MeBn)-Aib- | 2332.152 | 2332.159 | 9.8 | D |
| (SEQ ID | Leu-Ile-Cys]-Ttds-Lys(DOTA)-NH₂ | | | | |
| NO: 331) | | | | | |
| GIP-295 | Ac-Asp-Trp-Trp-Pro-Glu-Cha-[Cys(3MeBn)-Aib- | 2361.179 | 2361.187 | 9.8 | D |
| (SEQ ID | Leu-lle-Cys]-Ttds-Lys(DOTA)-NH₂ | | | | |
| NO: 332) | | | | | |
| GIP-296 | Ac-Asp-Trp-Trp-Pro-Glu-Aml-[Cys(3MeBn)-Aib- | 2335.163 | 2335.167 | 9.5 | D |
| (SEQ ID | Leu-Ile-Cys]-Ttds-Lys(DOTA)-NH₂ | | | | |
| NO: 333) | | | | | |
| GIP-297 | Ac-Asp-Nmw-Trp-Pro-Glu-Leu-[Cys(3MeBn)- | 2335.163 | 2335.170 | 9.4 | D |
| (SEQ ID | Aib-Leu-Ile-Cys]-Ttds-Lys(DOTA)-NH₂ | | | | |
| NO: 334) | | | | | |
| GIP-298 | Ac-1Ni-Pro-Glu-Leu-[Cys(3MeBn)-Aib-Leu-Ile- | 2031.046 | 2031.064 | 9.9 | D |
| (SEQ ID | Cys]-Ttds-Lys(DOTA)-NH₂ | | | | |
| NO: 335) | | | | | |
| GIP-299 | Nph1p-Pro-Glu-Leu-[Cys(3MeBn)-Aib-Leu-Ile- | 1974.025 | 1974.031 | 11.0 | D |
| (SEQ ID | Cys]-Ttds-Lys(DOTA)-NH₂ | | | | |
| NO: 336 | | | | | |
| GIP-300 | Ac-Asp-Trp-1Ni-Pro-Glu-leu-[Cys(3MeBn)-Ala- | 2304.121 | 2304.128 | 8.2 | D |
| (SEQ ID | Leu-val-Cys]-Ttds-Lys(DOTA)-NH₂ | | | | |
| NO: 337) | | | | | |
| GIP-301 | Ac-Trp-1Ni-Pro-Glu-Leu-[Cys(3MeBn)-Aib-Leu- | 2217.125 | 2217.133 | 10.2 | D |
| (SEQ ID | Ile-Cys]-Ttds-Lys(DOTA)-NH₂ | | | | |
| NO: 338) | | | | | |
| GIP-302 | Ac-Asp-Trp-Hyw-Pro-Glu-leu-[Cys(3MeBn)- | 2337.143 | 2337.145 | 8.2 | D |
| (SEQ ID | Aib-Leu-Ile-Cys]-Ttds-Lys(DOTA)-NH₂ | | | | |
| NO: 339) | | | | | |
| GIP-303 | Ac-Asp-7Nw-1Ni-Pro-Glu-leu-[Cys(3MeBn)- | 2333.148 | 2333.153 | 8.0 | D |
| (SEQ ID | Aib-Leu-Ile-Cys]-Ttds-Lys(DOTA)-NH₂ | | | | |
| NO: 340) | | | | | |
| GIP-304 | DOTA-Ttds-Trp-Asp-Pro-5Clw-leu-Glu- | 2141.997 | 2142.001 | 9.1 | D |
| (SEQ ID | [Cys(3MeBn)-Leu-Ile-Aib-AET] | | | | |
| NO: 341) | | | | | |
| GIP-305 | DOTA-Ttds-leu-Asp-Pro-5Clw-Trp-Leu- | 2141.997 | 2142.001 | 9.5 | D |
| (SEQ ID | [Cys(3MeBn)-Glu-Aib-Ile-AET] | | | | |
| NO: 342) | | | | | |
| GIP-306 | DOTA-Ttds-5Clw-Pro-Glu-leu-Asp-Trp- | 2141.997 | 2142.003 | 8.7 | D |
| (SEQ ID | [Cys(3MeBn)-Ile-Leu-Aib-AET] | | | | |
| NO: 343) | | | | | |
| GIP-307 | H-Met-Tyr-asp-Trp-Trp-Pro-Glu-Leu- | 1742.766 | 1742.778 | 9.5 | C |
| (SEQ ID | [Cys(3MeBn)-Ala-Leu-Ile-Cys]-NH₂ | | | | |
| NO: 344) | | | | | |
| GIP-308 | H-Met-Tyr-Asp-trp-Trp-Pro-Glu-Leu- | 1742.766 | 1742.771 | 10.1 | C |
| (SEQ ID | [Cys(3MeBn)-Ala-Leu-Ile-Cys]-NH₂ | | | | |
| NO: 345 | | | | | |
| GIP-309 | H-Met-Tyr-Asp-Trp-Trp-Pro-Glu-Leu- | 1742.766 | 1742.770 | 9.7 | C |
| (SEQ ID | [cys(3MeBn)-Ala-Leu-Ile-Cys]-NH₂ | | | | |
| NO: 346) | | | | | |
| GIP-310 | H-Met-Tyr-Asp-Trp-Trp-Pro-Glu-Leu- | 1742.766 | 1742.770 | 9.6 | C |
| (SEQ ID | [Cys(3MeBn)-Ala-Leu-Ile-cys]-NH₂ | | | | |
| NO: 347) | | | | | |
| GIP-311 | H-Met-Tyr-Asp-Trp-Trp-Ala-Glu-Leu- | 1716.750 | 1716.752 | 10.5 | C |
| (SEQ ID | [Cys(3MeBn)-Ala-Leu-Ile-Cys]-NH₂ | | | | |
| NO; 348) | | | | | |
| GIP-312 | H-Met-Tyr-Asp-Trp-Trp-Pro-Glu-Leu- | 1700.719 | 1700.723 | 9.2 | C |
| (SEQ ID | [Cys(3MeBn)-Ala-Ala-Ile-Cys]-NH₂ | | | | |
| NO: 349) | | | | | |
| GIP-313 | H-Met-Tyr-Asp-Trp-Trp-Pro-Glu-Leu- | 1742.766 | 1742.770 | 9.6 | C |
| (SEQ ID | [Cys(2MeBn)-Ala-Leu-Ile-Cys]-NH₂ | | | | |
| NO: 350) | | | | | |
| GIP-314 | HO-Succinyl-Trp-Trp-Pro-Glu-Leu- | 1433.651 | 1433.654 | 10.4 | C |
| (SEQ ID | [Cys(3MeBn)-ala-Leu-Ile-Cys]-NH₂ | | | | |
| NO: 351 | | | | | |
| GIP-315 | Ac-Asp-Trp-1Ni-Pro-Glu-Hle-[Cys(3MeBn)-Aib- | 1529.709 | 1529.712 | 12.0 | C |
| (SEQ ID | Leu-Ile-Cys]-NH₂ | | | | |
| NO: 352) | | | | | |
| GIP-316 | Ac-Cya-Gly-5Clw-Hyp-Glu-leu-[Cys(2Lut)-Aib- | 1419.543 | 1419.545 | 7.5 | C |
| (SEQ ID | Leu-Ile-AET] | | | | |
| NO: 353 | | | | | |
| GIP-317 | Ac-Cya-Trp-Trp-Pro-Glu-Leu-[Cys(3MeBn)-Ala- | 1526.640 | 1526.649 | 11.3 | C |
| (SEQ ID | Leu-Ile-Cys]-NH₂ | | | | |
| NO: 354) | | | | | |
| GIP-318 | HO-Succinyl-Trp-Trp-Pro-Glu-leu- | 1433.651 | 1433.657 | 10.2 | C |
| (SEQ ID | [Cys(3MeBn)-ala-Leu-Ile-Cys]-NH₂ | | | | |
| NO: 355) | | | | | |
| GIP-319 | HO-Succinyl-Trp-Trp-Hyp-Glu-Leu- | 1463.662 | 1463.665 | 11.3 | C |
| | [Cys(3MeBn)-Aib-Leu-Ile-Cys]-NH₂ | | | | |
| GIP-320 | Ac-Asp-Trp-Mcf-Hyp-Glu-leu-[Cys(2Lut)-Thp- | 1558.639 | 1558.653 | 9.4 | C |
| (SEQ ID | Leu-Ile-Cys]-NH₂ | | | | |
| NO: 356 | | | | | |
| GIP-322 | Ac-Asp-Trp-5Clw-Hyp-Glu-leu-[Cys(2Lut)-Thp- | 1571.598 | 1571.637 | 8.0 | C |
| (SEQ ID | Leu-Ser-Cys]-NH₂ | | | | |
| NO: 357) | | | | | |
| GIP-323 | Ac-Asp-Eaa-Eaa-Hyp-Glu-leu-[Cys(2Lut)-Thp- | 1621.511 | 1621.535 | 10.6 | C |
| (SEQ ID | Leu-Ile-Cys]-NH₂ | | | | |
| NO: 358) | | | | | |
| GIP-324 | Ac-Trp-Trp-Pro-Glu-Leu-[Cys(3MeBn)-Ala-Leu- | 1375.646 | 1375.648 | 11.0 | D |
| (SEQ ID | Ile-Cys]-NH₂ | | | | |
| NO: 359) | | | | | |
| GIP-325 | Ac-Trp-Pro-Glu-Leu-[Cys(3MeBn)-Ala-Leu-Ile- | 1189.566 | 1189.569 | 10.7 | D |
| (SEQ ID | Cys]-NH₂ | | | | |
| NO: 360) | | | | | |
| GIP-326 | Ac-Pro-Glu-Leu-[Cys(3MeBn)-Ala-Leu-Ile-Cys]- | 1003.487 | 1003.489 | 10.1 | D |
| (SEQ ID | NH₂ | | | | |
| NO: 361) | | | | | |
| GIP-327 | Ac-Glu-Leu-[Cys(3MeBn)-Ala-Leu-Ile-Cys]-NH₂ | 906.434 | 906.436 | 9.0 | D |
| (SEQ ID | | | | | |
| NO: 362) | | | | | |
| GIP-328 | H-Met-Tyr-Asp-Trp-Trp-pro-Glu-Leu- | 1742.766 | 1742.771 | 9.6 | D |
| (SEQ ID | [Cys(3MeBn)-Ala-Leu-Ile-Cys]-NH₂ | | | | |
| NO: 363) | | | | | |
| GIP-329 | H-Met-Tyr-Asp-Trp-trp-Pro-Glu-Leu- | 1742.766 | 1742.768 | 10.1 | D |
| (SEQ ID | [Cys(3MeBn)-Ala-Leu-Ile-Cys]-NH₂ | | | | |
| NO: 364 | | | | | |
| GIP-330 | H-Met-Tyr-Asp-Trp-Trp-Pro-Glu-Leu- | 1742.766 | 1742.768 | 8.9 | D |
| (SEQ ID | [Cys(3MeBn)-Ala-leu-Ile-Cys]-NH₂ | | | | |
| NO: 365) | | | | | |
| GIP-331 | H-Met-Tyr-Asp-Trp-Trp-Pro-Glu-Leu- | 1742.766 | 1742.770 | 9.8 | D |
| (SEQ ID | [Cys(4MeBn)-Ala-Leu-Ile-Cys]-NH₂ | | | | |
| NO: 366) | | | | | |
| GIP-332 | H-Met-Tyr-Ala-Trp-Trp-Pro-Glu-Leu- | 1698.776 | 1698.784 | 9.8 | D |
| (SEQ ID | [Cys(3MeBn)-Ala-Leu-Ile-Cys]-NH₂ | | | | |
| NO: 367) | | | | | |
| GIP-333 | H-Met-Tyr-Asp-Ala-Trp-Pro-Glu-Leu- | 1627.724 | 1627.727 | 9.1 | D |
| (SEQ ID | [Cys(3MeBn)-Ala-Leu-Ile-Cys]-NH₂ | | | | |
| NO: 368) | | | | | |
| GIP-334 | H-Met-Tyr-Asp-Trp-Ala-Pro-Glu-Leu- | 1627.724 | 1627.729 | 8.9 | D |
| (SEQ ID | [Cys(3MeBn)-Ala-Leu-Ile-Cys]-NH₂ | | | | |
| NO: 369) | | | | | |
| GIP-335 | H-Met-Tyr-Asp-Trp-Trp-Pro-Ala-Leu- | 1684.760 | 1684.765 | 10.0 | D |
| (SEQ ID | [Cys(3MeBn)-Ala-Leu-Ile-Cys]-NH₂ | | | | |
| NO: 370) | | | | | |
| GIP-336 | H-Met-Tyr-Asp-Trp-Trp-Pro-Glu-Ala- | 1700.719 | 1700.723 | 8.9 | D |
| (SEQ ID | [Cys(3MeBn)-Ala-Leu-Ile-Cys]-NH₂ | | | | |
| NO: 371) | | | | | |
| GIP-337 | H-Met-Tyr-Asp-Trp-Trp-Pro-Glu-Leu- | 1700.719 | 1700.723 | 8.9 | D |
| (SEQ ID | [Cys(3MeBn)-Ala-Leu-Ala-Cys]-NH₂ | | | | |
| NO: 372) | | | | | |
| GIP-338 | Ac-Asp-Trp-Trp-Pro-Glu-Leu-[Pen(3MeBn)-Aib- | 1532.720 | 1532.722 | 10.9 | D |
| (SEQ ID | Leu-Ile-Cys]-NH₂ | | | | |
| NO: 373) | | | | | |
| GIP-339 | Ac-Asp-Trp-Trp-Pro-Glu-Leu-[Cys(3MeBn)-Aib- | 1532.720 | 1532.722 | 10.4 | D |
| (SEQ ID | Leu-Ile-Pen]-NH₂ | | | | |
| NO: 374) | | | | | |
| GIP-340 | Ac-Asp-Trp-1Ni-Pro-Glu-Asn-[Cys(3MeBn)-Aib- | 1516.652 | 1516.655 | 8.6 | D |
| (SEQ ID | Leu-Ile-Cys]-NH₂ | | | | |
| NO: 375) | | | | | |
| GIP-341 | Ac-Asp-Trp-1Ni-4Dfp-Glu-Leu-[Cys(3MeBn)- | 1551.674 | 1551.676 | 11.8 | D |
| (SEQ ID | Aib-Leu-Ile-Cys]-NH₂ | | | | |
| NO: 376) | | | | | |
| GIP-342 | Ac-Asp-Trp-1Ni-Rni-Glu-Leu-[Cys(3MeBn)-Aib- | 1529.709 | 1529.712 | 10.9 | D |
| (SEQ ID | Leu-Ile-Cys]-NH₂ | | | | |
| NO: 377) | | | | | |
| GIP-343 | Ac-Asp-Trp-1Ni-Pro-Glu-Thi-[Cys(3MeBn)-Aib- | 1555.634 | 1555.636 | 11.1 | D |
| (SEQ ID | Leu-Ile-Cys]-NH₂ | | | | |
| NO: 378) | | | | | |
| GIP-344 | Ac-Asp-Trp-1Ni-Pro-Glu-Phe-[Cys(3MeBn)-Aib- | 1549.677 | 1549.679 | 11.4 | D |
| (SEQ ID | Leu-Ile-Cys]-NH₂ | | | | |
| NO: 379) | | | | | |
| GIP-345 | Ac-Asp-Trp-1Ni-Pro-Glu-Lys-[Cys(3MeBn)-Aib- | 1530.704 | 1530.711 | 8.2 | D |
| (SEQ ID | Leu-Ile-Cys]-NH₂ | | | | |
| NO: 380) | | | | | |
| GIP-346 | Ac-Asp-Trp-1Ni-Pro-Glu-Nle-[Cys(3MeBn)-Aib- | 1515.693 | 1515.697 | 10.2 | D |
| (SEQ ID | Leu-Ile-Cys]-NH₂ | | | | |
| NO: 381) | | | | | |
| GIP-347 | Ac-Asp-Trp-1Ni-Pro-Glu-Cit-[Cys(3MeBn)-Aib- | 1559.694 | 1559.698 | 8.8 | D |
| (SEQ ID | Leu-Ile-Cys]-NH₂ | | | | |
| NO: 382) | | | | | |
| GIP-348 | Ac-Asp-Trp-1Ni-Pro-Glu-Dap(Ac)-[Cys(3MeBn)- | 1530.668 | 1530.670 | 9.1 | D |
| (SEQ ID | Aib-Leu-Ile-Cys]-NH₂ | | | | |
| NO: 383) | | | | | |
| GIP-349 | Ac-Asp-Trp-Ocf-Hyp-Glu-leu-[Cys(2Lut)-Thp- | 1558.639 | 1558.649 | 9.1 | D |
| (SEQ ID | Leu-Ile-Cys]-NH₂ | | | | |
| NO: 384) | | | | | |
| GIP-350 | Ac-Asp-Trp-Pcf-Hyp-Glu-leu-[Cys(2Lut)-Thp- | 1558.639 | 1558.653 | 9.5 | D |
| (SEQ ID | Leu-Ile-Cys]-NH₂ | | | | |
| NO: 385) | | | | | |
| GIP-351 | Ac-Asp-Trp-Egm-Hyp-Glu-leu-[Cys(2Lut)-Thp- | 1592.600 | 1592.614 | 9.8 | D |
| (SEQ ID | Leu-Ile-Cys]-NH₂ | | | | |
| NO: 386) | | | | | |
| GIP-352 | Ac-Asp-Trp-Phe-Hyp-Glu-leu-[Cys(2Lut)-Thp- | 1524.678 | 1524.684 | 9.0 | D |
| (SEQ ID | Leu-Ile-Cys]-NH₂ | | | | |
| NO: 387) | | | | | |
| GIP-353 | Ac-Asp-Trp-5Clw-Hyp-Glu-leu-[Cys(2Lut)-Thp- | 1640.667 | 1640.682 | 7.0 | D |
| (SEQ ID | Leu-Arg-Cys]-NH₂ | | | | |
| NO: 388) | | | | | |
| GIP-354 | Ac-Asp-Trp-5Clw-Hyp-Glu-leu-[Cys(2Lut)-Thp- | 1613.609 | 1613.646 | 7.6 | D |
| (SEQ ID | Leu-Glu-Cys]-NH₂ | | | | |
| NO: 389) | | | | | |
| GIP-355 | Ac-Asp-Trp-5Clw-Hyp-Glu-leu-[Cys(2Lut)-Thp- | 1670.645 | 1670.691 | 8.9 | D |
| (SEQ ID | Leu-Trp-Cys]-NH₂ | | | | |
| NO: 390) | | | | | |
| GIP-356 | Ac-Asp-Eaa-Eaa-Oic-Glu-leu-[Cys(2Lut)-Thp- | 1659.563 | 1659.620 | 11.3 | D |
| (SEQ ID | Leu-Ile-Cys]-NH₂ | | | | |
| NO: 391) | | | | | |
| GIP-357 | Ac-Asp-Eaa-Eaa-Nmg-Glu-leu-[Cys(2Lut)-Thp- | 1579.501 | 1579.555 | 10.5 | D |
| (SEQ ID | Leu-Ile-Cys]-NH₂ | | | | |
| NO: 392) | | | | | |
| GIP-358 | DOTA-Ttds-Asp-Trp-5Clw-Pro-Glu-leu- | 2139.015 | 2139.027 | 8.3 | A |
| | [Dap(3MeBz)-Aib-Leu-Ile-AET] | | | | |
| GIP-359 | DOTA-Ttds-Asp-Trp-5Clw-Pro-Glu-leu- | 2139.015 | 2139.017 | 8.5 | B |
| | [Cys(3CbBn)-Aib-Leu-Ile-en] | | | | |
| GIP-360 | Ac-Asp-Trp-5Clw-Hyp-Glu-leu-[Cys(mli)-Thp- | 1587.593 | 1587.601 | 9.2 | A |
| | Leu-Ile-Cys]-NH₂ | | | | |
| GIP-361 | Ac-Asp-Trp-5Clw-Hyp-Glu-leu-[Cys(mli)-Thp- | 1601.609 | 1601.631 | 9.7 | A |
| | Leu-Ile-Hcy]-NH₂ | | | | |
| GIP-362 | Ac-Asp-Trp-5Clw-Hyp-Glu-leu-[Hcy(mli)-Thp- | 1601.609 | 1601.627 | 10.8 | B |
| | Leu-Ile-Cys]-NH₂ | | | | |
| GIP-363 | Ac-Asp-Trp-5Clw-Hyp-Glu-leu-[Hcy-Thp-Leu- | 1520.624 | 1520.639 | 10.6 | B |
| | Ile-Hcy]-NH₂ | | | | |
| GIP-364 | Ac-Asp-Trp-5Clw-Hyp-Glu-leu-[Cys-Thp-Leu- | 1492.592 | 1492.604 | 9.1 | C |
| (SEQ ID | Ile-Cys]-NH₂ | | | | |
| NO: 393) | | | | | |
| GIP-368 | H-Met-Tyr-Asp-Trp-Trp-Pro-Glu-Leu-Cys(Bzl)- | 1744.782 | 1744.790 | 9.6 | C |
| (SEQ ID | Ala-Leu-Ile-Smc-NH₂ | | | | |
| NO: 394) | | | | | |
| GIP-369 | H-Met-Tyr-Asp-Trp-Trp-Pro-Glu-Leu-Smc-Ala- | 1744.782 | 1744.789 | 9.8 | D |
| (SEQ ID | Leu-Ile-Cys(Bzl)-NH₂ | | | | |
| NO: 395) | | | | | |
| GIP-370 | AGLU-Glutar-Asp-Trp-5Clw-Tap-Glu-leu- | 2646.238 | 2646.232 | 5.5 | A |
| | [Cys(2Lut)-Apc(DOTAM)-Leu-Ile-Cys]-APAc- | | | | |
| | glu(AGLU)-NH₂ | | | | |
| GIP-371 | AGLU-Glutar-Asp-Trp-5Clw-Tap-Glu-leu- | 2665.221 | 2665.226 | 5.5 | A |
| | [Cys(2Lut)-Amk(DOTA)-Leu-Ile-Cys]-APAc- | | | | |
| | glu(AGLU)-NH₂ | | | | |
| GIP-372 | AGLU-Glutar-Asp-Trp-5Clw-Tap-Glu-leu- | 2633.195 | 2633.189 | 6.9 | A |
| | [Cys(2Lut)-Apc(DOTA)-Leu-Ile-Cys]-APAc- | | | | |
| | GLUSEROL | | | | |
| GIP-373 | NTASEROL-Asp-Trp-5Clw-Tap-Glu-leu- | 2804.271 | 2804.250 | 6.7 | A |
| | [Cys(2Lut)-Apc(DOTA)-Leu-Ile-Cys]-APAc- | | | | |
| | dap(NTASEROL)-NH₂ | | | | |
| GIP-374 | NTASEROL-Asp-Trp-5Clw-Tap-Glu-leu- | 2675.217 | 2675.208 | 6.6 | A |
| | [Cys(2Lut)-Apc(DOTA)-Leu-Ile-Cys]-APAc- | | | | |
| | GLUSEROL | | | | |
| GIP-375 | Ac-Asp-Trp-5Clw-Hyp-Lys-leu-[Cys(2Lut)-Thp- | 1596.702 | 1596.702 | 8.3 | A |
| | Leu-Ile-Cys]-NH₂ | | | | |
| GIP-376 | Ac-Asp-Trp-5Clw-Hyp-Arg-leu-[Cys(2Lut)-Thp- | 1624.709 | 1624.706 | 8.3 | A |
| | Leu-Ile-Cys]-NH₂ | | | | |
| GIP-377 | Ac-Asp-Trp-5Clw-Hyp-His-leu-[Cys(2Lut)-Thp- | 1605.666 | 1605.663 | 8.0 | A |
| | Leu-Ile-Cys]-NH₂ | | | | |
| GIP-378 | Ac-Asp-Trp-5Clw-Hyp-Hly-leu-[Cys(2Lut)-Thp- | 1610.718 | 1610.714 | 8.4 | A |
| | Leu-Ile-Cys]-NH₂ | | | | |
| GIP-379 | Ac-Asp-5Clw-5Clw-Hyp-Glu-leu-[Cys(2Lut)- | 1631.611 | 1631.613 | 8.1 | A |
| | Thp-Leu-Ile-Cys]-NH₂ | | | | |
| GIP-380 | Ac-Asp-1Ni-5Clw-Hyp-Glu-leu-[Cys(2Lut)-Thp- | 1608.655 | 1608.657 | 8.3 | A |
| | Leu-Ile-Cys]-NH₂ | | | | |
| GIP-381 | Ac-asp-Trp-5Clw-Hyp-Glu-leu-[Cys(2Lut)-Thp- | 1597.650 | 1597.654 | 7.7 | A |
| | Leu-Ile-Cys]-NH₂ | | | | |
| GIP-382 | Ac-Asp-5Fw-5Clw-Hyp-Glu-leu-[Cys(2Lut)-Thp- | 1615.641 | 1615.643 | 7.8 | A |
| | Leu-Ile-Cys]-NH₂ | | | | |
| GIP-383 | Ac-Asp-Trp-5Clw-Hyp-Glu-leu-[Cys(2Lut)-Thp- | 1637.681 | 1637.685 | 8.2 | A |
| | Cha-Ile-Cys]-NH₂ | | | | |
| GIP-384 | Ac-Asp-Trp-5Clw-Hyp-Ser-leu-[Cys(2Lut)-Thp- | 1555.640 | 1555.643 | 7.4 | A |
| | Leu-Ile-Cys]-NH₂ | | | | |
| GIP-385 | Ac-Asp-Trp-5Clw-Hyp-Val-leu-[Cys(2Lut)-Thp- | 1567.676 | 1567.679 | 8.4 | A |
| | Leu-Ile-Cys]-NH₂ | | | | |
| GIP-386 | Ac-Asp-Trp-5Clw-Hyp-Ala-leu-[Cys(2Lut)-Thp- | 1539.645 | 1539.644 | 7.9 | A |
| | Leu-Ile-Cys]-NH₂ | | | | |
| GIP-387 | DOTA-Ttds-Asp-Ocf-5Clw-Hyp-Glu-leu- | 2238.954 | 2238.959 | 7.3 | B |
| | [Cys(2Lut)-Thp-Leu-Ile-Cys]-NH₂ | | | | |
| GIP-388 | DOTA-Ttds-Asp-Trp-5Clw-Hyp-Glu-leu- | 2277.988 | 2277.985 | 7.1 | B |
| | [Cys(2Lut)-Thp-Leu-Phe-Cys]-NH₂ | | | | |
| GIP-389 | Ac-Asp-Trp-5Clw-Hyp-Glu-leu-[Cys(2Lut)-Thp- | 2145.910 | 2145.904 | 6.8 | B |
| | Leu-Phe-Cys]-lys(DOTA)-NH₂ | | | | |
| GIP-390 | Ac-Asp-Ocf-5Clw-Tap(DOTA)-Glu-leu- | 1977.796 | 1977.797 | 7.1 | B |
| | [Cys(2Lut)-Thp-Leu-Ile-Cys]-NH₂ | | | | |
| GIP-391 | Ac-Asp-Trp-5Clw-Tap(DOTA)-Glu-leu- | 2016.831 | 2016.833 | 6.8 | B |
| | [Cys(2Lut)-Thp-Leu-Phe-Cys]-NH₂ | | | | |
| GIP-392 | Ac-Asp-Ocf-5Clw-Hyp-Glu-leu-[Cys(2Lut)- | 1977.796 | 1977.800 | 7.6 | B |
| | Apc(DOTA)-Leu-Ile-Cys]-NH₂ | | | | |
| GIP-393 | Ac-Asp-Trp-5Clw-Hyp-Glu-leu-[Cys(2Lut)- | 2016.831 | 2016.831 | 7.2 | B |
| | Apc(DOTA)-Leu-Phe-Cys]-NH₂ | | | | |
| GIP-394 | Ac-Asp-Trp-1Ni-Tap(DOTA)-Glu-Hle- | 1930.900 | 1930.902 | 8.6 | B |
| | [Cys(3MeBn)-Aib-Leu-Ile-Cys]-NH₂ | | | | |
| GIP-395 | Ac-Asp-Trp-5Clw-Hyp-Har-leu-[Cys(2Lut)-Thp- | 1638.724 | 1638.720 | 8.5 | B |
| | Leu-Ile-Cys]-NH₂ | | | | |
| GIP-396 | Ac-Asp-Trp-5Clw-Hyp-Phe-leu-[Cys(2Lut)-Thp- | 1615.676 | 1615.680 | 8.6 | B |
| | Leu-Ile-Cys]-NH₂ | | | | |
| GIP-397 | Ac-Asp-Trp-5Clw-Hyp-Trp-leu-[Cys(2Lut)-Thp- | 1654.687 | 1654.689 | 8.3 | B |
| | Leu-Ile-Cys]-NH₂ | | | | |
| GIP-398 | Ac-Asp-Trp-5Clw-Hyp-Pro-leu-[Cys(2Lut)-Thp- | 1565.660 | 1565.659 | 7.5 | B |
| | Leu-Ile-Cys]-NH₂ | | | | |
| GIP-399 | Ac-Asp-Ocf-5Clw-Hyp-Glu-leu-[Cys(2Lut)-Thp- | 2106.875 | 2106.877 | 7.2 | C |
| | Leu-Ile-Cys]-lys(DOTA)-NH₂ | | | | |
| GIP-400 | Ac-Asp-Trp-1Ni-Pro-Glu-Hle-[Cys(3MeBn)- | 1956.915 | 1956.916 | 9.0 | C |
| | Apc(DOTA)-Leu-Ile-Cys]-NH₂ | | | | |
| GIP-401 | DOTA-Ttds-Asp-Trp-Egn-Hyp-Glu-leu- | 2238.954 | 2238.955 | 7.3 | B |
| | [Cys(2Lut)-Thp-Leu-Ile-Cys]-NH₂ | | | | |
| GIP-402 | Ac-Asp-Trp-Egn-Tap(DOTA)-Glu-leu-[Cys(2Lut)- | 1977.796 | 1977.797 | 7.1 | B |
| | Thp-Leu-Ile-Cys]-NH₂ | | | | |

*HPLC analyses were performed by HPLC/ELSD

### Example 30: Surface Plasmon Resonance (SPR) Based Binding Assay

Surface plasmon resonance (SPR) studies were performed using a Biacore™ T200 SPR system. Briefly, polarized light is directed towards a gold-labeled sensor surface, and minimum intensity reflected light is detected. The angle of reflected light changes as molecules bind and dissociate.

Biotinylate extracellular of the human GIP receptor (bioGIPR, Proteros biostructures GmbH, lot #02/2b) was captured on a biotin capture chip (Biotin CAPture Kit, Series S, GE Healthcare, Cat #28-9202-34). bioGIPR was diluted in Running Buffer (HBST from Xantec Analytics; 150 nM NaCl, 0.05% Tween20, 0.1% DMSO) to a final concentration 100 nM and than flushed over the Fc-capture chip to immobilized ˜3500 RUs.

Stock solutions of test compounds were prepared by dissolving each compound in DMSO. DMSO stock solution were diluted 1:1000 in Running Buffer without DMSO. Further sequential dilutions were made with Running Buffer containing 0.1% DMSO. SPR binding analyses were performed in Single Cycle Kinetic (SCK) mode at 25° C. Flow cell without immobilized bioGIPR served as reference flowcell. After each SCK run, bioGIRP and the Biotin Capture Reagent was removed with Regeneration Solution (6 M Guanidium, 0.75 M NaOH).

In between every three SCK measurements, a blank run with Running Buffer instead of test compound was included to correct for baseline drifts (double blanking method).

Table 9 describes the protocol steps for bioGIPR capturing and assessment of the binding kinetics.

TABLE 9
SPR protocol steps with bioGIPR.

| Step | Injected solution | Contact time | | Flow rate |
|---|---|---|---|---|
| Startup cycle (3x): | Running Buffer | 60 | s | 30 μL/min |
| Washing & surface regeneration | 6M Guanidin-HCl | 5 | s | |
| Immobilize Biotin Capture Reagent | Biotin Capture Reagent | 300 | s | 2 μL/min |
| (Streptavidin-oligo fusion) | | | | |
| Wash | Running Buffer | 60 | s | 30 μL/min |
| Capture bioGIPR | bioGIPR (100 nM) | 300 | s | 10 μL/min |
| 1. Binding kinetics of test compound | Dilution no. 5 (0.78 nM) | 120 | s | 30 μL/min |
| 2. Binding kinetics of test compound | Dilution no. 4 (3.125 nM) | 120 | s | 30 μL/min |
| 3. Binding kinetics of test compound | Dilution no. 3 (12.5 nM) | 120 | s | 30 μL/min |
| 4. Binding kinetics of test compound | Dilution no. 2 (50 nM) | 120 | s | 30 μL/min |
| 5. Binding kinetics of test compound | Dilution no. 1 (100 nM) | 120 | s | 30 μL/min |
| Dissociation cycle | Running Buffer | 2400 | s | 30 μL/min |
| Regeneration (2x) | Regeneration solution | 120 | s | 5 μL/min |

For each test compound, SPR raw data in the form of resonance units (RU) were plotted as sensorgrams using the Biacore™ T200 control software. The signal from the blank sensorgram was subtracted from that of the test compound sensorgram (blank corrected). The blank corrected sensorgram was corrected for baseline drift by subtracting the sensorgram of a SCK run without the test compound (running buffer only). The association rate (kₒₙ), dissociation rate (k_{off}), dissociation constant (K_{D}), and t_{1/2} were calculated from blank corrected SPR data using the 1:1 Langmuir binding model from the Biacore™ T200 evaluation software. Raw data and fit results were imported as text files in IDBS. The pK_{D} value (negative decadic logarithm of dissociation constant) was calculated in the IDBS excel template. FIG. 2 shows a representative sensorgram for compound PSM-01.

The results of this assay for a selection of compounds according to the present invention are presented in Table 10. pK_{D} category A stands for pK_{D} values>9.0, category B for pK_{D} values between 8.1 and 9.0, category C for pK_{D} values between 7.1 and 8.0, and category D for pK_{D}values≤7.0. t_{1/2} category A stands for t_{1/2} values>15 min, category B for t_{1/2} between 15 min and 5.1 min, category C for t_{1/2} between 5 min and 2.1 min, and category D for t_{1/2}<2.0 min.

The results of this assay for a selection of compounds according to the present invention are presented in Table 10.

TABLE 10
Compound ID, pK_{D}-category and t_{1/2}-category.

| Compound ID | pK_{D} Category | t_{1/2} Category |
|---|---|---|
| GIP-001 | A | A |
| GIP-002 | A | A |
| GIP-004 | A | A |
| GIP-005 | A | A |
| GIP-006 | A | A |
| GIP-007 | A | A |
| GIP-008 | A | A |
| GIP-010 | A | A |
| GIP-011 | A | A |
| GIP-021 | A | A |
| GIP-022 | A | A |
| GIP-046 | B | D |
| GIP-048 | B | D |
| GIP-049 | B | D |
| GIP-065 | A | A |
| GIP-066 | A | A |
| GIP-067 | A | A |
| GIP-068 | A | A |
| GIP-069 | A | A |
| GIP-072 | A | A |
| GIP-077 | A | A |
| GIP-079 | A | A |
| GIP-095 | A | A |
| GIP-096 | A | A |
| GIP-097 | A | A |
| GIP-103 | A | A |
| GIP-106 | B | A |
| GIP-107 | A | A |
| GIP-108 | A | A |
| GIP-109 | A | A |
| GIP-110 | A | A |
| GIP-111 | B | A |
| GIP-112 | A | A |
| GIP-113 | A | A |
| GIP-114 | A | A |
| GIP-117 | A | A |
| GIP-118 | A | A |
| GIP-119 | A | A |
| GIP-121 | A | A |
| GIP-142 | B | A |
| GIP-146 | A | A |
| GIP-151 | A | A |
| GIP-153 | A | A |
| GIP-155 | A | A |
| GIP-167 | A | A |
| GIP-169 | A | A |
| GIP-179 | A | A |
| GIP-188 | A | A |
| GIP-194 | A | A |
| GIP-197 | A | A |
| GIP-359 | B | C |
| GIP-372 | A | A |
| GIP-373 | A | A |
| GIP-374 | A | A |
| GIP-375 | B | A |
| GIP-376 | B | A |
| GIP-377 | B | A |
| GIP-378 | B | A |
| GIP-395 | B | A |

### Example 31: Plasma Stability Assay

In order to determine the stability of selected compounds of the invention in human and mouse plasma, a plasma stability assay was carried out. Such plasma stability assay measures degradation of compounds of the present invention in blood plasma. This is an important characteristic of a compound as compounds, with the exception of pro-drugs, which rapidly degrade in plasma, generally show poor in vivo efficacy. The results show that those compounds are highly stable in human and mouse plasma. The stability is sufficient for the diagnostic, therapeutic and theragnostic use of these compounds according to the present invention.

The plasma stability samples were prepared by spiking 50 μL plasma aliquots (all K2EDTA) with 1 μL of a 0.5 mM compound stock solution in DMS0. After vortexing the samples were incubated in a Thermomixer at 37° C. for 0, 4 and 24 hours. After incubation the samples were stored on ice until further treatment. All samples were prepared in duplicates.

A suitable internal standard was added to each sample (1 μL of a 0.5 mM stock solution in DMSO). Protein precipitation was performed by addition of 250 μL of acetonitrile containing 1% trifluoroacetic acid. After incubation at room temperature for 30 min the precipitate was separated by centrifugation and 150 μL of the supernatant was diluted with 150 μL of 1% aqueous formic acid.

The determination of the analyte in the clean sample solutions was performed on an Agilent 1290 UHPLC system coupled to an Agilent 6530 Q-TOF mass spectrometer. The chromatographic separation was carried out on a Phenomenex BioZen XB—C18 HPLC column (50×2 mm, 1.7 μm particle size) with gradient elution using a mixture of 0.10% formic acid in water as eluent A and acetonitrile as eluent B (2% B to 41% in 7 min, 800 μL/min, 40° C.). Mass spectrometric detection was performed in positive ion ESI mode by scanning the mass range from m/z 50 to 3000 with a sampling rate of 2/sec.

From the mass spectrometric raw data, the ion currents for the double or triple charged monoisotopic signal was extracted for both, the compound and the internal standard.

Quantitation was performed by external matrix calibration with internal standard using the integrated analyte signals.

Additionally, recovery was determined by spiking a pure plasma sample that only contained the internal standard after treatment with a certain amount of the compound.

Carry-over was evaluated by analysis of a blank sample (20% acetonitrile) after the highest calibration sample.

The results of this assay performed on some of the compounds according to the present invention are given in the following Table 11. The result is stated as “% intact compound remaining after 4 h or 24 h” and means that from the amount of material at the start of the experiment the stated percentage is detected as unchanged material at the end of the experiment by LC-MS quantification. Stability category A stands for >80% intact after 24 hours, category B stands for >80% intact after 4 hours, category C stands for ≥50% intact after 4 hours and category D stands for <50% intact after 4 hours. Compounds with more than 50% intact after at least 4 h they are considered as stable enough for diagnostic and therapeutic applications.

TABLE 11
Results of the plasma stability assay

| | Human plasma stability | Mouse plasma stability |
|---|---|---|
| Compound | category | category |
| GIP-001 | A | A |
| GIP-005 | n.d. | A |
| GIP-126 | n.d. | B |
| GIP-127 | n.d. | B |
| GIP-128 | n.d. | B |
| GIP-129 | n.d. | B |
| GIP-240 | B | B |
| GIP-241 | B | C |
| GIP-242 | B | C |
| GIP-244 | B | B |
| GIP-245 | B | D |
| GIP-246 | B | B |
| GIP-247 | C | D |
| GIP-248 | B | D |
| GIP-270 | n.d. | B |

### Example 32: Proteolytic Stability Assay Against NEP

Peptides are often sensitive to proteolytic cleavage in the blood (Werle et al., Amino Acids, 2006, 30, 351-367). If peptides degrade rapidly, in vivo elimination could be dominated by metabolism. Since metabolites often demonstrate poor binding to the target, performance of the compound during radiopharmaceutical use (diagnostic or therapeutic) can be significantly decreased. Therefore, evaluating the stability of the compound against proteases in the early stages of compound development is essential.

Two classes of proteases and peptidases can be found in the blood circulation: soluble and membrane-bound proteases. The conduction of a plasma stability study only covers the impact of the soluble fraction on compound stability, since all membrane-bound proteases are separated during blood collection (proteins expressed on the surface of blood vessels) or during plasma generation (proteins expressed on blood cells). Thus, the result of a plasma stability assay is not always predictive for in vivo behavior.

Membrane-bound peptidases are mainly responsible for the activation and deactivation of bioactive peptides, such as hormones, cytokines, and neurohormones (Antczak et al., Bioessays, 2001, 23, 251-260). They activate hormones by cleavage of a pre-hormone and deactivate them through another cleavage step. Activation is usually very specific, as well as the activation peptidases, including ACE, ECE, and DPPIV. With a specific activation, signaling remains specific even if deactivation is more unspecific. Thus, several peptidases that are mainly responsible for hormone deactivation reveal a broad substrate pattern (e.g. neutral endopeptidase, meprines). It is not possible to assess all peptidases during early compound development. Therefore, more importance should be focused on those peptidases with broad substrate patterns, since it is more likely that the peptide sequence fits into this pattern.

NEP (neutral endopeptidase, EC 3.4.24.11, CD10, CALLA, endopeptidase 24.11, enkephalinase, neprilysin, membrane metallopeptidase A) is a membrane-bound metallopeptidase. It is expressed on neutrophils and highly active in blood (Antczak et al., Bioessays, 2001, 23, 251-260). In addition, the cleavage pattern of NEP is very broad, covering many different peptide hormones, with more than 50 different natural substrates already described (Bayes-Genis et al., Journal of the American College of Cardiology, 2016, 68, 639-653). NEP preferably cleaves amide bonds between a hydrophilic and hydrophobic amino acid (preferably leucine or phenylalanine), even in small cyclic peptides such as CNP. Plamboeck et al. (2005) showed a significant impact of NEP caused cleavage on the pharmacokinetic behavior of peptides (Plamboeck et al., Diabetologia, 2005, 48, 1882-1890).

To assess the stability of compounds against NEP, the compounds were subjected to an in vitro assay based on the protocol described by Edelson et al. (Edelson et al., Pulmonary pharmacology & therapeutics, 2013, 26, 229-238).

Recombinant soluble human NEP (BioTechne, Wiesbaden, Germany) at a concentration of 100 ng/mL was mixed with the compound (10 μM) and a stable internal standard (10 μM) and incubated at 37° C. After several time points, samples were taken and analyzed with LC-MS.

The determination of the analyte in the clean sample solutions was performed on an Agilent 1290 UHPLC system coupled to an Agilent 6530 Q-TOF mass spectrometer. The chromatographic separation was carried out on a Phenomenex BioZen XB—C18 HPLC column (50×2 mm, 1.7 μm particle size) with gradient elution using a mixture of 0.1% formic acid in water as eluent A and acetonitrile as eluent B (2% B to 410% in 7 min, 800 μL/min, 40° C.). Mass spectrometric detection was performed in positive ion ESI mode by scanning the mass range from m/z 50 to 3000 with a sampling rate of 2/sec.

From the mass spectrometric raw data the ion currents for the double or triple charged monoisotopic signal was extracted for both, the compound and the internal standard.

Quantitation was performed by external matrix calibration with internal standard using the integrated analyte signals.

The activity of NEP was examined using a commercially available chromogenic substrate of NEP.

The results of this assay performed on some of the compounds according to the present invention are given in the following Table 12. The result is stated as “% intact compound remaining after 24 h incubation” and means that from the amount of material at the start of the experiment the stated percentage is detected as unchanged material at the end of the experiment by LC-MS quantification. Stability category A stands for >80% intact after 24 hours, category B stands for >50% intact after 24 hours, category C stands for ≥50% intact after 4 hours and category D stands for <50% intact after 4 hours. Compounds with more than 50% intact after at least 24 h they are considered as stable enough for diagnostic and therapeutic applications.

Since all compounds are more than 50% intact after 24 h they are considered as stable enough for diagnostic and therapeutic applications.

TABLE 12
Results of the NEP stability assay

| Compound | NEP stability category |
|---|---|
| GIP-001 | A |
| GIP-003 | A |
| GIP-039 | A |
| GIP-040 | A |
| GIP-126 | A |
| GIP-127 | A |
| GIP-128 | A |
| GIP-244 | A |
| GIP-270 | A |

### Example 33: ¹¹¹In- and ¹⁷⁷Lu-Labeling of Selected Compounds

In order to serve as a diagnostically, therapeutically, or theragnostically active agent, a compound needs to be labeled with a radioactive isotope. The labeling procedure needs to be appropriate to ensure a high radiochemical yield and purity of the radiolabeled compound of the invention. This example shows that the compounds of the present invention are appropriate for radiolabeling and can be labeled in high radiochemical yield and purity.

30-105 MBq of ¹¹¹InCl₃ (in 0.02 M HCl) were mixed with 1 nmol of compound (200 μM stock solution in 0.1 M HEPES pH 7) per 30 MBq and buffer (A: 1 M sodium acetate buffer pH 5, containing 20 mg/mL gentisic acid, B: 1 M sodium acetate/ascorbic acid buffer pH 5 containing 25 mg/mL methionine or C: 1 M sodium acetate buffer pH 5 containing 25 mg/mL methionine) at a final buffer concentration of 0.1 M. The mixture was heated to 80° C. for 25 min. After cooling down, DTPA and TWEEN-20 were added at a final concentration of 0.2 mM and 0.1%, respectively (Formulation A). In some instances, 20 μL of a 200 mg/mL ascorbic acid solution per 100 μL reaction mixture was additionally added at the end of synthesis (Formulation B).

2.0±0.1 GBq ¹⁷⁷LuCl₃ (in 0.04 M HCl) were mixed with 1 nmol of compound (200 μM stock solution in 0.1 M HEPES pH 7) per 60-90 MBq and buffer (1 M sodium acetate/ascorbic acid buffer pH 5 containing 25 mg/mL methionine) at a final buffer concentration of ˜0.4 M. The mixture was heated for 20 min at 90° C. After cooling down, DTPA and TWEEN-20 were added at a final concentration of 0.2 mM and 0.1%, respectively (Formulation A). In some instances, 9 volumes of a 100 mg/mL ascorbic acid solution containing 5 mg/mL methionine and 0.8 mM DTPA were added instead (Formulation C).

Radiochemical purity was analyzed by HPLC. 5 μL of diluted labeling solution was analyzed with a Poroshell SB-C18 2.7 μm (Agilent). Eluent A: H₂O, 0.1% TFA, eluent B: MeCN; gradient from 5% B to 70% B within 15 min, flow rate 0.5 mL/min; detector: NaI (Raytest), DAD 230 nm. The peak eluting with the dead volume represents free radionuclide, the peak eluting with the peptide-specific retention time as determined with an unlabeled sample represents radiolabeled compound.

Radiochemical purity was ≥92% at end of synthesis. Exemplary radiochemical purities for selected ¹¹¹In-labeled compounds are shown in Table 13. ¹⁷⁷Lu-labeled compounds maintained a radiochemical purity of ≥85% up 6 days post synthesis (Table 13).

TABLE 13
Radiochemical purity of 111 In-labeled
compounds as measured by HPLC.

| | | | HPLC | HPLC Area |
|---|---|---|---|---|
| | Labeling | | retention | % at end of |
| | buffer | Formulation | time [min] | synthesis |
| ¹¹¹In-GIP-001 | B | A | 8.8 | >97.0 |
| ¹¹¹In-GIP-002 | B | A | 9.1 | >97.0 |
| ¹¹¹In-GIP-003 | B | A | 8.6 | 99.0 |
| ¹¹¹In-GIP-003 | C | B | 8.6 | 96.6 |
| ¹¹¹In-GIP-004 | A | A | 8.9 | 97.9 |
| ¹¹¹In-GIP-005 | B | A | 8.8 | 98.2 |
| ¹¹¹In-GIP-005 | C | B | 8.8 | 98.3 |
| ¹¹¹In-GIP-006 | B | A | 8.4 | 99.3 |
| ¹¹¹In-GIP-007 | B | A | 8.2 | 100.0 |
| ¹¹¹In-GIP-008 | C | B | 8.3 | >99.0 |
| ¹¹¹In-GIP-009 | C | B | 8.7 | 97.4 |
| ¹¹¹In-GIP-010 | B | A | 9.0 | 98.3 |
| ¹¹¹In-GIP-011 | C | B | 8.8 | 95.5 |
| ¹¹¹In-GIP-012 | B | A | 8.8 | 97.6 |
| ¹¹¹In-GIP-015 | C | B | 8.9 | 98.2 |
| ¹¹¹In-GIP-016 | C | B | 8.8 | 98.4 |
| ¹¹¹In-GIP-017 | C | B | 8.9 | 97.8 |
| ¹¹¹In-GIP-018 | C | B | 8.6 | 98.9 |
| ¹¹¹In-GIP-019 | C | B | 8.9 | 98.8 |
| ¹¹¹In-GIP-020 | C | B | 8.8 | 98.2 |
| ¹¹¹In-GIP-021 | B | A | 9.8 | 96.5 |
| ¹¹¹In-GIP-022 | B | A | 11.2 | 98.2 |
| ¹¹¹In-GIP-023 | C | B | 9.4 | 98.8 |
| ¹¹¹In-GIP-077 | A | A | 11.6 | >97.0 |
| ¹¹¹In-GIP-079 | A | A | 11.2 | 98.4 |
| ¹¹¹In-GIP-080 | A | A | 11.7 | 92.9 |
| ¹¹¹In-GIP-082 | A | A | 11.3 | 99.5 |
| ¹¹¹In-GIP-088 | A | A | 11.3 | 98.0 |
| ¹¹¹In-GIP-089 | A | A | 11.2 | 98.5 |
| ¹¹¹In-GIP-093 | A | A | 10.7 | 97.4 |
| ¹¹¹In-GIP-094 | A | | 11.3 | >98.2 |
| ¹¹¹In-GIP-095 | A | A | 10.8 | 97.5 |
| ¹¹¹In-GIP-096 | A | A | 10.9 | 97.1 |
| ¹¹¹In-GIP-097 | A | A | 11.6 | 97.5 |
| ¹¹¹In-GIP-103 | A | A | 10.0 | 96.1 |
| ¹¹¹In-GIP-106 | A | A | 9.6 | >98.3 |
| ¹¹¹In-GIP-109 | A | A | 10.0 | >98.3 |
| ¹¹¹In-GIP-112 | A | A | 9.7 | 98.5 |
| ¹¹¹In-GIP-142 | B | A | 11.2 | 99.3 |
| ¹¹¹In-GIP-359 | A | A | 11.7 | 96.2 |

Radiochemical purity of ¹⁷⁷Lu-labeled compounds as measured by HPLC.

| | | | | HPLC Area |
|---|---|---|---|---|
| | | HPLC | HPLC Area | % appr. 6 d |
| | | retention | % at end of | post end of |
| | Formulation | time [min] | synthesis | synthesis |
| ¹⁷⁷Lu-GIP-001 | C | 8.8 | 97.4 | 94.8 |
| ¹⁷⁷Lu-GIP-002 | A | 9.1 | 97.7 | 85.0 |

### Example 34: Imaging Studies

Radioactively labeled compounds can be detected by imaging methods such as SPECT and PET. Furthermore, the data acquired by such techniques can be confirmed by direct measurement of radioactivity contained in the individual organs prepared from an animal injected with a radioactively labeled compound of the invention. Thus, the biodistribution (the measurement of radioactivity in individual organs) of a radioactively labeled compound can be determined and analyzed. This example shows that the compounds of the present invention show a biodistribution appropriate for diagnostic imaging of tumors.

All animal experiments were conducted in compliance with the German animal protection laws. Male SCID beige mice (6-to 8-week-old, Charles River Laboratories, France) were inoculated with 5×10⁶ target-positive CHO-K1-hGIPR (GenScript Biotech, #M00486) and target negative HT-29 cells (DSMZ, Germany) subcutaneously on opposite shoulders in the same mouse. When tumors reached a size between 150 mm³ and 500 mm³ mice received ˜30 MBq ¹¹¹In-labeled compounds of the invention (diluted to 100 μL with PBS) administered intravenously via the tail vein. Images were obtained on a NanoSPECT/CT system (Mediso Medical Imaging Systems, Budapest, Hungary) using exemplarily the following acquisition and reconstruction parameters (Table 14).

TABLE 14
Acquisition and reconstruction parameters
of NanoSPECT/CT imaging
Acquisition parameters SPECT

| System | NanoSPECT/CT ™ | |
|---|---|---|
| Scan range | whole body, 3-bed holder | |
| | (mouse hotel) | |
| Time per projection | 60 | s |

| | |
|---|---|
| Aperture model, pinhole | Aperture #2, 1,5 mm |
| diameter | |

Reconstruction parameters

| | |
|---|---|
| Method | HiSPECT (Scivis), |
| | iterative reconstruction |
| Smoothing | 35% |
| Iterations | 9 |
| Voxel size | 0.15 mm × 0.15 mm × 0.15 mm |

Acquisition parameters CT

| System | NanoSPECT/CT TM | |
|---|---|---|
| Scan range | whole body, 3-bed holder | |
| | (mouse hotel) | |
| Scan duration | 7 | min |
| Tube voltage | 45 | kVp |
| Exposure time | 500 | ms |

| | |
|---|---|
| Number of projections | 240 |

Imaging data were saved as DICOM files and analyzed using VivoQuant™ software (Invicro, Boston, USA). Results are expressed as a percentage of injected dose per gram of tissue (0% ID/g). Two animals were treated with each compound.

Table 15 states the tumor and kidney uptake after 1 h p.i. to 24 h p.i. calculated as area under the curve (AUC) (GraphPad Prism). Tumor uptake category A stands for AUC>250 ID/g, category B for AUC between 250% ID/g and 150% ID/g, and category C for AUC<150% ID/g. Kidney uptake category A stands for AUC<150% ID/g, category B for AUC between 150% ID/g and 200% ID/g, and category C for AUC>200% ID/g.

The results of the imaging studies for selected compounds are shown in FIGS. 3, 4 and 5.

TABLE 15

| Compound ID | AUC_{1-24 h} Tumor | AUC_{1-24 h} Kidneys |
|---|---|---|
| ¹¹¹In-GIP-001 | A | A |
| ¹¹¹In-GIP-002 | A | A |
| ¹¹¹In-GIP-003 | A | A |
| ¹¹¹In-GIP-004 | A | B |
| ¹¹¹In-GIP-005 | A | B |
| ¹¹¹In-GIP-006 | A | A |
| ¹¹¹In-GIP-007 | B | A |
| ¹¹¹In-GIP-008 | A | A |
| ¹¹¹In-GIP-009 | B | A |
| ¹¹¹In-GIP-010 | A | A |
| ¹¹¹In-GIP-011 | A | A |
| ¹¹¹In-GIP-012 | B | A |
| ¹¹¹In-GIP-013 | B | A |
| ¹¹¹In-GIP-014 | B | A |
| ¹¹¹In-GIP-015 | A | A |
| ¹¹¹In-GIP-016 | A | A |
| ¹¹¹In-GIP-017 | B | B |
| ¹¹¹In-GIP-018 | B | C |
| ¹¹¹In-GIP-019 | A | B |
| ¹¹¹In-GIP-020 | B | C |
| ¹¹¹In-GIP-021 | A | C |
| ¹¹¹In-GIP-022 | A | A |
| ¹¹¹In-GIP-023 | A | C |
| ¹¹¹In-GIP-077 | A | A |
| ¹¹¹In-GIP-079 | A | B |
| ¹¹¹In-GIP-080 | B | A |
| ¹¹¹In-GIP-109 | A | C |
| ¹¹¹In-GIP-142 | C | A |

### Example 35: Radioligand Binding Assay

In order to assess the affinity of ¹¹¹In-labeled compounds to the GIPR receptor of different species, a radioligand binding assay was established.

CHO-K1 cells (GenScript Biotech, #M00486) were stably transfected with the human GIPR. CHO-K1-hGIPR cells were cultured in Ham's F12 medium (Sigma-Aldrich, #N4888) including 10% fetal calf serum (FCS), 2 mM L-glutamine, 100 U/mL penicillin, 100 g/mL streptomycin, 200 g/mL Zeocin and 100 g/mL HygromycinB. To evaluate the species cross-reactivity, CHO-VGT cells were transfected with the human, dog, rat and mouse GIPR (Inscreenex GmbH), which were cultured in DMEM/Ham's F12 Medium 1:1 (Biochrom, #F485) including 5% fetal calf serum (FCS), 2 mM L-glutamine, 100 U/mL penicillin and 100 μg/mL streptomycin. Cells were detached with Accutase (Biolegend, #BLD-423201) and counted using a particle counter (CASY Model TT; Schärfe Systems). Cell concentrations were adjusted to 3×10⁵ mL⁻¹, and 1.000 μL of the suspension per well were dispensed into flat-clear-bottom 24 well plates.

Approximately 24 hours after re-seeding, the medium was aspirated and the cells were washed once with 700 μL assay medium (Ham's medium with 20 mM HEPES). To determine total binding, 700 μL of assay medium and 100 μL of the radioligand dilutions were added to the wells in triplicates. To determine non-specific binding, 600 μL of binding medium, 100 μL of the radioligand dilutions and 100 μL of a blocking solution containing 8 μM non-labeled compound were added to the wells in triplicates.

For determination of the equilibrium time, cells were incubated with 0.1 nM and 0.2 nM radiolabeled compound for 1 h, 3 h, 6 h, and 8 h at 37° C. under standard cell culture conditions (5% CO₂). For determination of the dissociation constant (K_{D}) cells were incubated with increasing concentrations of ¹¹¹In-labeled compound radiolabeled compound for 6 h at 37° C. under standard cell culture conditions (5% CO₂).

At the end of incubation time, cells were washed and subsequently harvested in 300 μL RIPA buffer containing PIC. Radioactivity of the cell lysate of each well was counted using a gamma counter (Perkin Elmer). An aliquot of each radioligand dilution was included in the gamma counter measurements to allow for determination of their actual concentrations via their known specific activity.

The results of the radioligand binding assay are shown in Table 16. pK_{D} category A stands for pK_{D} values>9.5, category B for pK_{D} between 9.0 and 9.5 and category C for pK_{D}<9.0.

TABLE 16
Results of the radioligand binding assay

| Compound ID | Species | Category |
|---|---|---|
| GIP-001 | Human GIPR | A |
| GIP-001 | Dog GIPR | A |
| GIP-001 | Rat GIPR | B |
| GIP-001 | Mouse GIPR | C |
| GIP-002 | Human GIPR | A |

### Example 36: cAMP Assay

In order to determine the inhibitory or stimulatory activity of compounds according to the present invention of GIPR-expressing cells, a cAMP activity assay was established.

HEK-GIPR cells were generated by InSCREENeX GmbH (Germany) using the targeting vector pTAR to insert the minimal CMV promoter upstream of the human GIPR cDNA by recombinase-mediated cassette exchange into the pre-tagged HEK293 cell line.

HEK-GIPR cells were cultured in RPMI-1640 medium (Sigma-Aldrich, #R0883) including 10% fetal calf serum (FCS), 8 mM L-glutamine, 100 U/mL penicillin, 100 μg/mL streptomycin and 1 mg/mL Geneticin. Cells were detached with Accutase (Biolegend, #BLD-423201) and diluted in culture medium to a final concentration of 100,000 cells per mL. 100 μL of the cell suspension were transferred to white poly-L-lysine-coated 96-well plates with optical bottom (Nunc, #165306) and incubated overnight in a humidified atmosphere containing 5% CO₂ at 37° C.

For EC₅₀ determination, cells were incubated in 20 μL induction buffer (RPMI-1640 growth medium including 100 μM Ro 20-1724 (Sigma-Aldrich, #B8279) and 500 μM IBMX (Sigma-Aldrich, #I7018)) containing various concentrations of compound at 37° C. for 30 min.

For IC₅₀ determination, cells were incubated in 20 μL induction buffer (RPMI-1640 growth medium including 100 μM Ro 20-1724 (Sigma-Aldrich, #B8279) and 500 μM IBMX (Sigma-Aldrich, #I7018)) containing various concentrations of antagonistic compound and 3 μM GIP(1-30) agonist (H-YAEGTFISDYSIAMDKIHQQDFVNWLLAQK-NH₂; 3BP in-house synthesis) at 37° C. for 30 min.

For cAMP detection, the cAMP-Glo™ Assay (Promega, #V1501) was employed. Following the incubation period, 20 μL cAMP Lysis Buffer (Promega, #V1501) was added to each well and the plate was shaken at 1,000 RPM for 15 min at room temperature. 40 μL of the cAMP detection solution (Promega, #V1501) was added to each well and the plate was shaken at 850 RPM for 20 min at room temperature. 80 μL of the Kinase-Glo-Reagent Solution (Promega, #V1501) was added to each well and mixed at 850 RPM for 30 sec. After 10 min of incubation, luminescence was read using a SpectraMax M5 Plate Reader (Molecular Devices).

Relative luminescence values (RLU) were plotted against peptide concentration and four parameter logistic (4PL) curve fitting and calculations of EC₅₀/pEC₅₀ and IC₅₀/pIC₅₀, respectively, were performed using ActivityBase software.

The result of the EC₅₀ assay is shown in Table 17 and the combined results of the EC₅₀ and IC₅₀ assays in Table 18.

TABLE 17

| Compound ID/ | | |
|---|---|---|
| description | Sequence | Mean pEC₅₀ ± SD |
| GIP(1-30) | H-Tyr-Ala-Glu-Gly-Thr-Phe-Ile-Ser-Asp- | 11.8 ± 0.1 |
| | Tyr-Ser-Ile-Ala-Met-Asp-Lys-Ile-His-Gln- | |
| | Gln-Asp-Phe-Val-Asn-Trp-Leu-Leu-Ala-Gln- | |
| | Lys-NH₂ | |
| GIP(1-30) | H-Tyr-Ala-Glu-Gly-Thr-Phe-Ile-Ser-Asp- | 12.1 ± 0.1 |
| C-ter. DOTA | Tyr-Ser-Ile-Ala-Met-Asp-Lys-Ile-His-Gln- | |
| | Gln-Asp-Phe-Val-Asn-Trp-Leu-Leu-Ala-Gln- | |
| | Lys(DOTA-Ahx)-NH₂ | |
| GIP(1-30) | H-Tyr-Ala-Glu-Gly-Thr-Phe-Ile-Ser-Asp- | 12.0 ± 0.1 |
| C-ter. ^{nat}InDOTA | Tyr-Ser-Ile-Ala-Met-Asp-Lys-Ile-His-Gln- | |
| | Gln-Asp-Phe-Val-Asn-Trp-Leu-Leu-Ala-Gln- | |
| | Lys(^{nat}InDOTA-Ahx)-NH₂ | |

TABLE 18

| Compound ID | pIC₅₀ Category | pEC₅₀ Category |
|---|---|---|
| GIP-001 | A | D* |
| GIP-002 | A | D* |
| GIP-028 | B | D* |
| GIP-065 | A | D* |
| GIP-067 | A | D* |
| GIP-071 | A | D* |

*Not stimulatory in a wide concentration range between 0.1 pM and 10 μM. Full antagonist.

The disclosure of each and any references recited herein is incorporated herein by reference.

1. A compound comprising a cyclic peptide of formula (Ia) 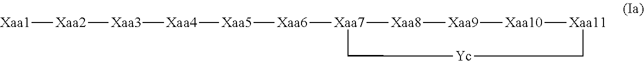
or a cyclic peptide of formula (Ib) 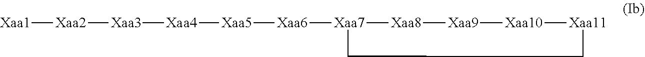
each optionally comprising an N-terminal modification group A attached to Xaa1, and each optionally comprising a C-terminal group C-term attached to Xaa11,
wherein
the peptide sequence is drawn from left to right in N-to C-terminal direction,
Xaa1 is a residue of an α-amino acid comprising a side chain, wherein the side chain comprises an acidic or polar group, or
Xaa1 is a residue of an aliphatic carboxylic acid comprising an acidic or polar group at the ω-position,
wherein the carbonyl group of Xaa1 is covalently attached to the α-nitrogen atom of Xaa2,
Xaa2 is a residue of an α-amino acid comprising a side chain, wherein the side chain comprises a heteroaryl ring or an aryl ring, preferably Xaa2 is a residue of an α-amino acid with a side chain comprising a heteroaryl ring,
wherein the carbonyl group of Xaa2 is covalently attached to the α-nitrogen atom of Xaa3,
Xaa3 is a residue of an α-amino acid comprising a side chain, wherein the side chain comprises a heteroaryl ring or an aryl ring, preferably Xaa3 is a residue of an α-amino acid comprising a side chain, wherein the side chain comprises a heteroaryl ring,
wherein the carbonyl group of Xaa3 is covalently attached to the α-nitrogen atom of Xaa4,
Xaa4 is a residue of an α-amino acid optionally comprising a Z group or is a residue of an N-alkylated α-amino acid optionally comprising a Z group,
wherein the carbonyl group of Xaa4 is covalently attached to the α-nitrogen atom of Xaa5,
Xaa5 is a residue of an α-amino acid optionally comprising a Z group,
wherein the carbonyl group of Xaa5 is covalently attached to the α-nitrogen atom of Xaa6,
Xaa6 is a residue of an α-amino acid comprising a side chain, wherein the side chain comprises a branched aliphatic group,
wherein the carbonyl group of Xaa6 is covalently attached to the α-nitrogen atom of Xaa7,
Xaa7 is a residue of an α-amino acid comprising a side chain, wherein the side chain comprising a nucleophilic group, wherein the nucleophilic group comprises a heteroatom, wherein the heteroatom is selected from the group comprising a sulfur atom and a nitrogen atom,
wherein the carbonyl group of Xaa7 is covalently attached to the α-nitrogen atom of Xaa8,
Xaa8 is a residue of an α-amino acid optionally comprising a Z group,
wherein the carbonyl group of Xaa8 is covalently attached to the α-nitrogen atom of Xaa9,
Xaa9 is a residue of an α-amino acid,
wherein the carbonyl group of Xaa9 is covalently attached to the α-nitrogen atom of Xaa10,
Xaa10 is a residue of an α-amino acid comprising a side chain, wherein the side chain comprises a branched aliphatic group or an unsubstituted aromatic ring,
wherein the carbonyl group of Xaa10 is covalently attached to the α-nitrogen atom of Xaa11, if Xaa11 is a residue of an α-amino acid comprising a side chain, wherein the side chain comprises a nucleophilic group, wherein the nucleophilic group comprises a heteroatom, wherein the heteroatom is selected from the group comprising a sulfur atom and a nitrogen atom, or, if Xaa11 is a residue of an aliphatic bis-nucleophile, wherein the first nucleophile of the bis-nucleophile is a nitrogen atom and the second nucleophile of the bis-nucleophile is a heteroatom, wherein the heteroatom is selected from the group comprising a sulfur atom and a nitrogen atom, to the first nucleophile,
Xaa11 is a residue of an α-amino acid comprising a side chain, wherein the side chain comprises a nucleophilic group, wherein the nucleophilic group comprises a heteroatom, wherein the heteroatom is selected from the group comprising a sulfur atom and a nitrogen atom, or
Xaa11 is a residue of an aliphatic bis-nucleophile, wherein the first nucleophile of the bis-nucleophile is a nitrogen atom and the second nucleophile of the bis-nucleophile is a heteroatom, wherein the heteroatom is selected from the group comprising a sulfur atom and a nitrogen atom,
wherein Xaa7 and Xaa11 are indirectly linked or directly covalently linked to each other forming a macrocyclic ring,
wherein if Xaa7 and Xaa11 are indirectly linked forming a cyclic peptide of formula (Ia), the heteroatom of Xaa7 and the heteroatom of Xaa11 are linked through a ring interspersed between the heteroatom of Xaa7 and the heteroatom of Xaa11 forming Yc, wherein the ring is a heteroaryl ring, an aryl ring optionally comprising a Z group or a heterocyclic ring, wherein the heteroatom is each and individually selected from the group consisting of a sulfur atom and a nitrogen atom, wherein if the heteroatom of Xaa7 is a sulfur atom, a thioether bond covalently links Xaa7 to the ring, if the heteroatom of Xaa7 is a nitrogen atom, an amine bond or an amide bond covalently links Xaa7 to the ring, if the heteroatom of Xaa11 is a sulfur atom, a thioether bond covalently links Xaa11 to the ring, and if the heteroatom of Xaa11 is a nitrogen atom, an amine bond or an amide bond covalently links Xaa11 to the ring, or
wherein if Xaa7 and Xaa11 are directly covalently linked forming a cyclic peptide of formula (Ib), the heteroatom of Xaa7 is a sulfur atom and the heteroatom of Xaa11 is a sulfur atom forming a disulfide bond,
and wherein
if present, the N-terminal modification group A is selected from the group comprising
a) a Z group, wherein a linker is optionally interspersed between the Z group and Xaa1,
b) a blocking group AblN,
c) a residue of an amino acid optionally comprising a Z group and optionally comprising a blocking group AblN, and
d) an N-terminal dipeptide group comprising a residue of a first amino acid and a residue of a second amino acid, wherein the residue of the first amino acid is the N-terminal amino acid residue and the residue of the second amino acid is the C-terminal amino acid residue,
wherein the residue of the first amino acid optionally comprises a blocking group AblN,
wherein the residue of the first amino acid and the residue of the second amino acid are covalently attached to each other, preferably by a peptide bond,
wherein the residue of the second amino acid is covalently attached to the residue of Xaa1 or a linker is interspersed between the second amino acid and the residue of Xaa1,
and wherein a Z group is optionally covalently attached to the residue of the first amino acid and/or a Z group is optionally covalently attached to the residue of the second amino acid,
and wherein
if present, the C-terminal group C-term is selected from the group comprising
a) a residue of an amino acid,
wherein the residue of an amino acid is covalently attached to Xaa11 or a linker is interspersed between the residue of an amino acid and Xaa11,
wherein a Z group is optionally covalently attached to the residue of an amino acid,
and wherein the residue of an amino acid comprises a blocking group AblC,
b) a C-terminal dipeptide group, wherein the C-terminal dipeptide group comprises a residue of a first amino acid and a residue of a second amino acid, wherein the residue of the first amino acid is the N-terminal amino acid residue and the residue of the second amino acid is the C-terminal amino acid residue,
wherein the residue of the first amino acid and the residue of the second amino acid are covalently attached to each other, preferably by a peptide bond,
wherein the residue of the first amino acid is covalently attached to the residue of Xaa11 or a linker is interspersed between the residue of the first amino acid and the residue of Xaa11,
wherein a Z group is optionally covalently attached to the residue of the first amino acid and/or a Z group is optionally covalently attached to the residue of the second amino acid,
and wherein the residue of the second amino acid comprises a blocking group AblC;
c) a Z group; and
d) a blocking group AblC.

2. The compound of claim 1,
wherein
Xaa1 is (a) a residue of an α-amino acid comprising a side chain, wherein the side chain comprises an acidic or polar group, or
(b) a residue of an aliphatic carboxylic acid comprising an acidic or polar group at the ω-position, preferably Xaa1 is a residue of an amino acid of formulae (IIa), (IIb) or (IIc), more preferably Xaa1 is a residue of an amino acid formula (IIa), 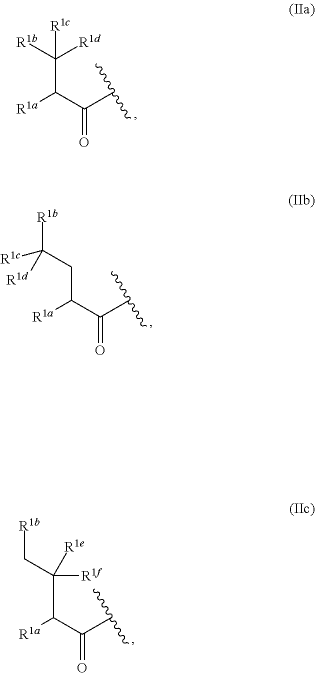
wherein
R^{1a} is selected from the group consisting of —NH—, H, —NH((C₁-C₄)alkyl)-, OH, and (C₁-C₄)alkyl,
wherein if R^{1a} is selected from the group consisting of —NH— and —NH((C₁-C₄)alkyl)-, the N-terminal modification group A is covalently attached to the nitrogen atom of R^{1a},
and if R^{1a} is selected from the group consisting of H, OH or (C₁-C₄)alkyl, the N-terminal modification group A is absent,
R^{1b} is selected from the group consisting of CO₂H, SO₃H, OPO₃H₂, CONH₂ and OH,
R^{1c} is selected from the group consisting of H, OH and (C₁-C₂)alkyl under the proviso that R^{1b} is selected from the group consisting of CO₂H, SO₃H and CONH₂,
or is selected from the group consisting of H and (C₁-C₂)alkyl under the proviso that R^{1b} is selected from the group consisting of OH and OPO₃H₂,
R^{1d} is selected from the group consisting of H and (C₁-C₂)alkyl, preferably if R^{1c} is (C₁-C₂)alkyl, R^{1d} is the same (C₁-C₂)alkyl,
R^{1e} is selected from the group consisting of H, OH and (C₁-C₂)alkyl,
R^{1f} is selected from the group consisting of H and (C₁-C₂)alkyl, preferably if R^{1e} is (C₁-C₂)alkyl, R^{1f} is the same (C₁-C₂)alkyl,
and the carbonyl group of Xaa1 is covalently attached to the α-nitrogen atom of Xaa2,
Xaa2 is (a) a residue of an α-amino acid comprising a side chain, wherein the side chain comprises a heteroaryl ring or an aryl ring, preferably Xaa2 is a residue of an amino acid of formulae (IIIa) or (IIIb), more preferably a residue of an amino acid of formula (IIIa), 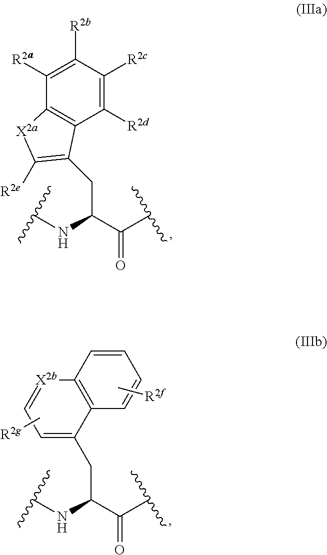
wherein
X^{2a} is selected from the group consisting of NH, NCH₃ and S, preferably selected from the group consisting of NH and S,
X^{2b} is selected from the group consisting of N, CH, CF and C—CH₃, preferably selected from the group consisting of N and CH,
R^{2a} is selected from the group consisting of H, a halogen, methyl and OCH₃, preferably R^{2a} is selected from the group consisting of H and F,
R^{2b} is selected from the group consisting of H, a halogen, methyl and OCH₃, preferably the halogen is selected from the group consisting of Cl and Br, more preferably R^{2b} is selected from the group consisting of H, Cl and Br,
R^{2c} is selected from the group consisting of H, OH, a halogen, methyl and OCH₃, preferably the halogen is selected from the group consisting of F, Cl and Br, more preferably R², is selected from the group consisting of H, F and OH,
R^{2d} is selected from the group consisting of H, a halogen, methyl and OCH₃, wherein the halogen is selected from the group consisting of F, Cl and Br, preferably R^{2d} is H,
R^{2e} is selected from the group consisting of H, a halogen and methyl, preferably the halogen is Cl, more preferably R^{2e} is H,
R^{2f} is selected from the group consisting of H and OCH₃, preferably R^{2f} is H,
R^{2g} is selected from the group consisting of H, OH, methyl and OCH₃, preferably R^{2g} is H,
or (b), under the proviso that Xaa3 is of formulae (IVa) or (IVb), preferably under the proviso that Xaa3 is of formula (IVa), Xaa2 is a residue of an α-amino acid comprising a side chain, wherein the side chain comprises an aryl ring, preferably Xaa2 is a residue of an amino acid of formula (IIIc) 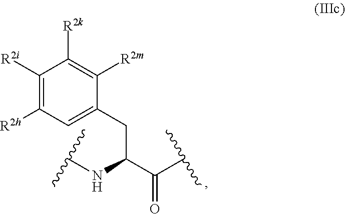
wherein
R^{2h} is selected from the group consisting of H, methyl, CF₃, OH, OCH₃, OCF₃, CONH₂ and a halogen, preferably the halogen is selected from the group consisting of Cl and Br, more preferably R^{2h} is H, Cl or Br,
R²ⁱ is selected from the group consisting of H, methyl, C(CH₃)₃, CF₃, OH, OCH₃, OCH₂CH₃, OCF₃, CONH₂, CO₂H and a halogen, preferably the halogen is selected from the group consisting of Cl and Br, more preferably R²ⁱ is selected from the group consisting of H, Cl and Br,
R^{2k} is selected from the group consisting of H, methyl, CF₃, OH, OCH₃, OCF₃, CONH₂ and a halogen, preferably the halogen is selected from the group consisting of Cl and Br, more preferably R^{2k} is selected from the group consisting of H, Cl and Br,
R^{2m} is selected from the group consisting of H, methyl, CF₃, OH, OCH₃, OCF₃, CONH₂ and a halogen, preferably the halogen is selected from the group consisting of Cl and Br, more preferably R^{2m} is selected from the group consisting of H, Cl and Br,
and the carbonyl group of Xaa2 is covalently attached to the α-nitrogen atom of Xaa3,
Xaa3 is (a) a residue of an α-amino acid comprising a side chain, wherein the side chain comprises a heteroaryl ring or an aryl ring, preferably Xaa3 is a residue of an amino acid of formulae (IVa) or (IVb), more preferably Xaa3 is a residue of an amino acid of formula (IVa), 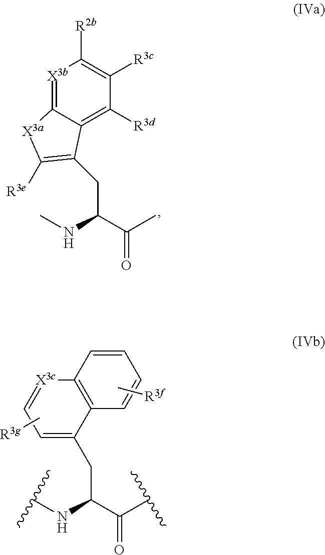
wherein
X^{3a} is selected from the group consisting of NH, NCH₃ and S, preferably selected from the group consisting of NH and S,
X^{3b} is selected from the group consisting of C—H, C—F, C—Cl, C—Br, C—CH₃, C—OCH₃ and N, preferably X^{3b} is C—H or C—F,
X^{3c} is selected from the group consisting of C and N, preferably X^{3c} is C,
R^{3b} is selected from the group consisting of H, a halogen, methyl and OCH₃, preferably the halogen is selected from the group consisting of F and Cl, more preferably R^{3b} is selected from the group consisting of H and F,
R^{3c} is selected from the group consisting of H, a halogen, methyl and OCH₃, preferably the halogen is selected from the group consisting of Cl, Br and F, more preferably R^{3c} is selected from the group consisting of Cl and Br,
R^{3d} is selected from the group consisting of H, a halogen, methyl and OCH₃, preferably the halogen is F,
R^{3e} is selected from the group consisting of H, a halogen and methyl, preferably the halogen is Cl,
R^{3f} is selected from the group consisting of H and OCH₃,
R^{3g} is selected from the group consisting of H, F, OH, methyl and OCH₃,
or (b) under the proviso that Xaa2 is an amino acid residue of formula (IIIa), Xaa3 is a residue of an α-amino acid comprising a side chain, wherein the side chain comprises an aryl ring, preferably Xaa3 is a residue of an amino acid of formulae (IVc) or (IVd) 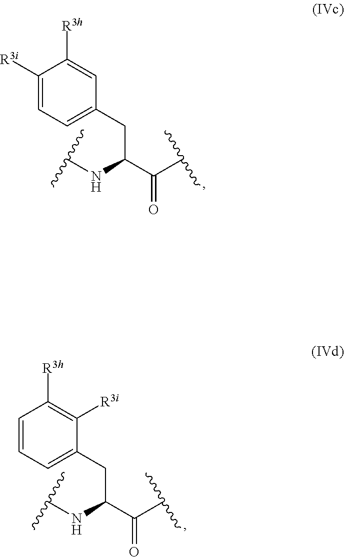
wherein
R^{3h} is selected from the group consisting of a halogen, OH, methyl, CF₃, OCH₃, OCF₃ and CONH₂, preferably the halogen is selected from the group consisting of Cl and Br,
R³ⁱ is selected from the group consisting of a halogen, OH, methyl, CF₃, OCH₃, OCF₃ and CONH₂, preferably the halogen is selected from the group consisting of Cl and Br,
and the carbonyl group of Xaa3 is covalently attached to the α-nitrogen atom of Xaa4,
Xaa4 is (a) a residue of a cyclic non-aromatic α-amino acid, preferably Xaa4 is a residue of an amino acid of formula (Va) 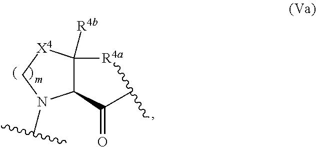
wherein
R^{4a} is selected from the group consisting of H, OH, methyl and CF₃,
R^{4b} is selected from the group consisting of H and methyl, preferably R^{4b} is methyl if R^{4a} is methyl,
X⁴ is selected from the group consisting of CHR^{4c}, S and O,
wherein R^{4c} is absent or is selected from the group consisting of NH₂, OH, H, NHR^{4d}, methyl and F, preferably R^{4c} is NH₂ or OH,
wherein R^{4d} is selected from the group consisting of Ac and a Z group,
m is 1 or 2, preferably m is 1,
or (b) a residue of an N-alkylated amino acid, preferably Xaa4 is a residue of an amino acid of formula (Vb) 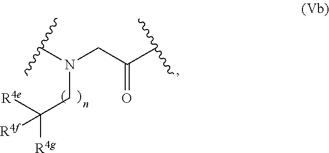
wherein
n is 0, 1, 2, 3, 4, or 5, preferably n is 0, 1 or 3,
R^{4e} is, if n is 0, selected from the group consisting of H, methyl, an aryl ring, COOH, CONH₂ and C(═O)R^{4h},
or is, if n is 1, 2, 3, 4 or 5, selected from the group consisting of H, methyl, an aryl ring, OH, NH₂, COOH, CONH₂ and C(═O)R^{4h},
R^{4f} is selected from the group consisting of H and (C₁-C₂)alkyl,
R^{4g} is selected from the group consisting of H and methyl,
R^{4h} is a Z group, preferably the Z group is a bio-distribution modifier, more preferably the bio-distribution modifier is AGLU,
or (c) Xaa4 is a residue of a bicyclic non-aromatic amino acid, preferably Xaa4 is a residue of an amino acid of formula (Vc) 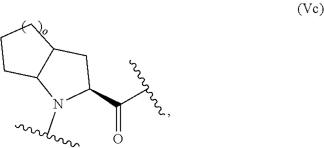
wherein
o is 1 or 2,
and the carbonyl group of Xaa4 is covalently attached to the α-nitrogen atom of Xaa5,
Xaa5 is (a) a residue of an α-amino acid optionally comprising a Z group, wherein the α-nitrogen atom of the α-amino acid is optionally substituted by (C₁-C₄)alkyl or (b) a cyclic α-amino acid, and the carbonyl group of Xaa5 is covalently attached to the α-nitrogen atom of Xaa6,
Xaa6 is a residue of an α-amino acid comprising a side chain, wherein the side chain comprises a branched aliphatic group, preferably Xaa6 is a residue of an amino acid of formula (VIIa) 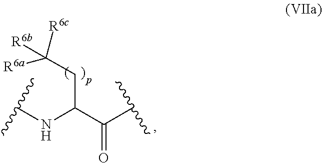
wherein
p is 1, 0 or 2, preferably p is 1,
R^{6a} is selected from the group consisting of (C₁-C₂)alkyl,
R^{6b} is methyl,
R^{6c} is H, if p is 0,
or is selected from the group consisting of H and methyl, if p is 1 or 2,
and the carbonyl group of Xaa6 is covalently attached to the α-nitrogen atom of Xaa7,
Xaa7 is a residue of an α-amino acid comprising a side chain, wherein the side chain comprising a nucleophilic group, wherein the nucleophilic group comprises a heteroatom, wherein the heteroatom is selected from the group comprising a sulfur atom and a nitrogen atom, preferably Xaa7 is a residue of an α-amino acid of formula (VIIIa) 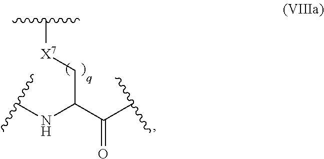
wherein
X⁷ comprises the heteroatom and is selected from the group consisting of —S— and —NH—,
q is 1 or 2, preferably q is 1,
and the carbonyl group of Xaa7 is covalently attached to the α-nitrogen atom of Xaa8,
Xaa8 is (a) a residue of an aliphatic cyclic α-amino acid, of an aliphatic heterocyclic α-amino acid or of an aliphatic cyclic α-amino acid comprising an annulated aromatic ring, preferably Xaa8 is a residue of an amino acid of formula (IXa) or (IXb), more preferably Xaa8 is a residue of an amino acid of formula (IXa), 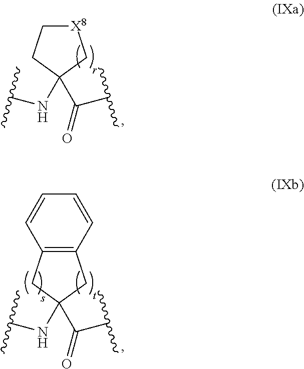
wherein
X⁸ is selected from the group consisting of NR^{8a}, O, NH, and CH₂, wherein R^{8a} is a Z group or R^{8a} is acetyl,
r is 2 or 1, preferably r is 2,
s is 1 or 2, preferably s is 1,
t is 1 or 2, preferably t is 1 if s is 1, and t is 2 if s is 2,
or (b) a residue of an α-amino acid or an α-alkyl-α-amino acid, wherein preferably Xaa8 is a residue of an amino acid of formula (IXc), 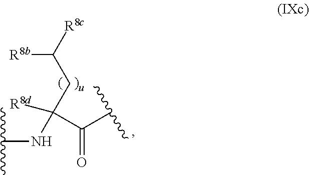
wherein
R^{8b} is selected from the group consisting of H, OH, NH₂, NHR^{8e}, (C₁-C₂)alkyl, COOH, CONH₂, an aryl ring and a heteroaryl ring, preferably the aryl ring is phenyl and the heteroaryl ring is 3-indoyl, wherein R^{8e} is a Z group, preferably the Z group is a chelator optionally comprising a linker,
R^{8c} is selected from the group consisting of H and methyl, if R^{8b} is selected from the group consisting of OH, NH₂ and NHR^{8e},
or is selected from the group consisting of H, methyl and OH, if R^{8b} is selected from the group consisting of H, (C₁-C₂)alkyl, COOH, CONH₂, an aryl ring and a heteroaryl ring,
R^{8d} is selected from the group consisting of H and (C₁-C₂)alkyl,
u is 0, 1, 2, 3, 4, or 5, preferably u is 0, 1 or 3,
and the carbonyl group of Xaa8 is covalently attached to the α-nitrogen atom of Xaa9,
Xaa9 is a residue of an acyclic α-amino acid, whereby the acyclic α-amino acid is in L-configuration and the carbonyl group of Xaa9 is covalently attached to the α-nitrogen atom of Xaa10,
Xaa10 is a residue of an α-amino acid comprising a side chain, wherein the side chain comprises a branched aliphatic group or an unsubstituted aromatic ring, preferably Xaa10 is a residue of an α-amino acid of formula (XIa), 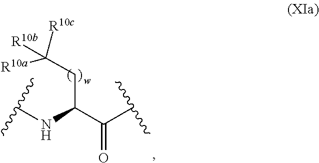
wherein
R^{10a} is selected from the group consisting of (C₁-C₂)alkyl and phenyl,
R^{10b} is selected from the group consisting of methyl and H,
R^{10c} is selected from the group consisting of H and methyl,
w is 0 or 1,
and the carbonyl group of Xaa10 is covalently attached to the α-nitrogen of Xaa11, if Xaa11 is a residue of an α-amino acid comprising a side chain, wherein the side chain comprises a nucleophilic group, wherein the nucleophilic group comprises a heteroatom, wherein the heteroatom is selected from the group comprising S and N, or, if Xaa11 is a residue of an aliphatic bis-nucleophile, wherein the first nucleophile of the bis-nucleophile is a nitrogen atom and the second nucleophile of the bis-nucleophile is a heteroatom, wherein the heteroatom is selected from the group comprising S and N, to the first nucleophile,
Xaa11 is (a) a residue of an α-amino acid comprising a side chain, wherein the side chain comprises a nucleophilic group, wherein the nucleophilic group comprises a heteroatom, wherein the heteroatom is selected form the group comprising a sulfur atom and a nitrogen atom,
or (b) Xaa11 is a residue of an aliphatic bis-nucleophile, wherein the first nucleophile of the bis-nucleophile is a nitrogen atom and the second nucleophile of the bis-nucleophile is a heteroatom, wherein the heteroatom is selected from the group comprising a sulfur atom and a nitrogen atom,
preferably Xaa11 is of formula (XIIa) 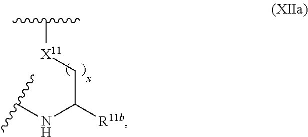
wherein
X¹¹ comprises the heteroatom and is selected from the group consisting of —S— and —NH—,
R^{11b} is selected from the group consisting of C(═O)R^{11c}, CONH₂, CO₂H, CH₂OH and H, wherein R^{11c} is C-term,
x is 1 or 2,
and wherein if R^{1a} in Xaa1 is selected from the group consisting of —NH— and —NH((C₁-C₄)alkyl)-, the N-terminal modification group A is covalently attached to the nitrogen atom in R^{1a}, wherein the N-terminal modification group A is
a) a Z group, wherein a linker is optionally interspersed between the Z group and Xaa1, or
b) a blocking group AblN, wherein the blocking group AblN is selected from the group consisting of acetyl, a structure of formula (AblN-Ia) and Nph2p, 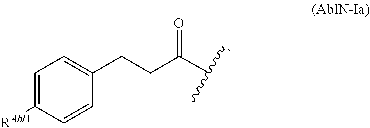
wherein
R^{Abl1} is selected from the group consisting of H, OH and a halogen, wherein the halogen is preferably selected from the group consisting of F and Cl, preferably R^{Abl1} is OH
preferably AblN is of formula (Abl-Ia) or is acetyl, more preferably Abl is acetyl, or
c) the residue of an amino acid as defined in claim 1, wherein the amino acid is an α-amino acid,
wherein the residue of an amino acid optionally comprises a Z group covalently attached to the side chain of the residue of an amino acid and optionally comprises a Z group covalently attached to the α-amino group of the residue of an amino acid, or
wherein the residue of an amino acid optionally comprises a blocking group AblN covalently attached to the α-amino group of the residue of an amino acid, preferably the blocking group AblN is acetyl,
and wherein the carbonyl group of the residue of an amino acid is covalently attached to the nitrogen atom in R^{1a} of residue Xaa1,
and
d) the N-terminal dipeptide group as defined in claim 1,
wherein the residue of the first amino acid, the residue of the second amino acid or both the residues of the first and of the second amino acid optionally comprise a Z group,
wherein an optional blocking group AblN is covalently attached to the α-amino group of the residue of the first amino acid,
and wherein a linker is optionally interspersed between the residue of the second amino acid and the residue of Xaa1,
wherein if the interspersed linker is absent, the carbonyl group of the residue of the first amino acid is covalently attached to the α-nitrogen of the residue of the second amino acid and the carbonyl group of the residue of the second amino acid is covalently attached to the nitrogen atom in R^{1a},
or wherein if the interspersed linker is present, the carbonyl group of the residue of the first amino acid is covalently attached to the α-nitrogen of the residue of the second amino acid and the carbonyl group of the residue of the second amino acid is covalently attached to a nitrogen atom of the linker and a carbonyl group of the linker is covalently attached to the nitrogen atom in R^{1a},
wherein if Xaa11 is a residue of an amino acid of formula (XIIa), wherein R^{11b} is C(═O)R^{11c}, and R^{11c} is the C-terminal group C-term, C-term is selected from the group comprising
a) the residue of an amino acid as defined in claim 1, wherein the amino acid is an α-amino acid,
optionally comprising a Z group and optionally comprising a linker interspersed between the residue of Xaa11 and the residue of an amino acid,
wherein if the interspersed linker is absent, the carbonyl group R^{11b} of residue Xaa11 is covalently attached to the α-nitrogen of the residue of an amino acid,
or wherein if the interspersed linker is present, the carbonyl group in R^{11b} of residue Xaa11 is covalently attached to a nitrogen atom of the linker, a carbonyl group of the linker is covalently attached to the α-nitrogen of the residue of an amino acid,
and wherein a blocking group AblC is covalently attached to the carbonyl group of the residue of an amino acid, wherein the blocking group AblC is selected from the group consisting of NH₂ and OH, wherein the blocking group AblC preferably is NH₂, or,
b) the C-terminal dipeptide group,
wherein the residue of the first amino acid, the residue of the second amino acid or both the residue of the first and of the second amino acid optionally comprise a Z group,
and wherein a linker is optionally interspersed between the residue of Xaa11 and the residue of the first amino acid,
wherein if the interspersed linker is absent, the carbonyl group R^{11b} of residue Xaa11 is covalently attached to the α-nitrogen of the residue of the first amino acid and the carbonyl group of the residue of the first amino acid is covalently attached to the α-nitrogen of the residue of the second amino acid,
or wherein if the interspersed linker is present, the carbonyl group R^{11b} of residue Xaa11 is covalently attached to a nitrogen atom of the linker, a carbonyl group of the linker is covalently attached to the α-nitrogen of the residue of the first amino acid and the carbonyl group of the residue of the first amino acid is covalently attached to the α-nitrogen of the residue of the second amino acid,
and wherein a blocking group Abl is covalently attached to the carbonyl group of the residue of the second amino acid, wherein the blocking group AblC is selected from the group consisting of NH₂ and OH, wherein the blocking group AblC preferably is NH₂,
and
c) a Z group.

3. The compound of claim 1,
wherein
(a)
Xaa7 is a residue of formula (VIIIa) 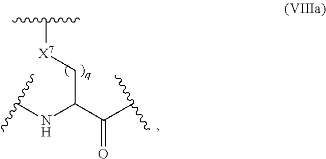
wherein
X⁷ is —S— or —NH—,
q is 1 or 2, preferably q is 1,
Yc is a structure of formula (XIIIa) 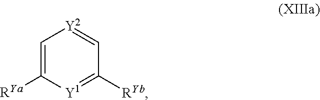
wherein
R^{Ya} is —CH₂—,
R^{Yb} is —CH₂—,
Y¹ is selected from the group consisting of N and CH,
Y² is selected from the group consisting of CH and N,
and Xaa11 is a residue of formula (XIIa) 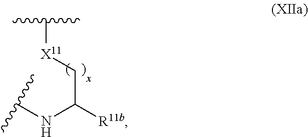
wherein
X¹¹ is —S— or —NH—,
R^{11b} is selected from the group consisting of C(═O)R^{11c}, CONH₂, CO₂H, CH₂OH and H, wherein R^{11c} is the C-terminal group C-term,
x is 1 or 2, preferably x is 1,
preferably the compound comprises a cyclic peptide of formula (Ic), 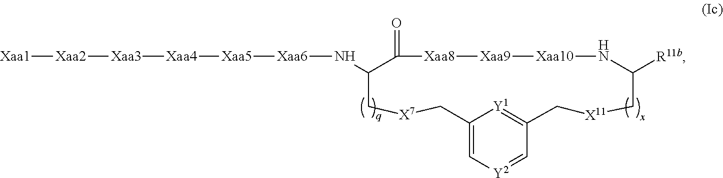
more preferably the compound comprises a cyclic peptide of formula (Ic),
wherein X⁷ is —S—, X¹¹ is —S—, Y¹ is N or CH, Y² is CH, q is 1, and x is 1,
most preferably the compound comprises a cyclic peptide of formula (Ic),
wherein X⁷ is —S—, X¹¹ is —S—, Y¹ is N, Y² is CH, q is 1, and x is 1,
(b)
Xaa7 is a residue of formula (VIIIa) 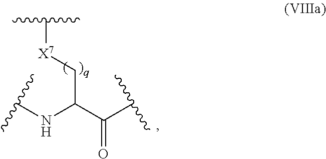
wherein
X⁷ is —S— or —NH—,
q is 1 or 2, preferably q is 1,
Yc is a structure of formula (XIIIh) 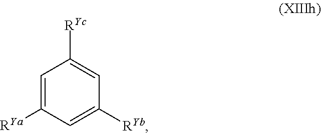
wherein
R^{Ya} is —CH₂—,
R^{Yb} is —CH₂—, and
R^{Yc} is —CH₂—R^{Yd}, and
R^{Yd} is a structure of formulae (XIIIc), (XIIId) or (XIIIe), 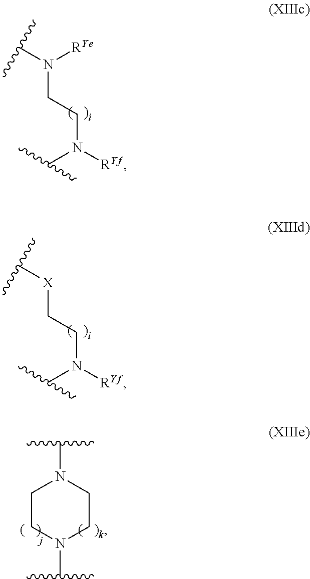
wherein
R^{Ye} and R^{f} are each and independently selected from the group consisting of H and (C₁-C₄)alkyl,
i is each and independently 1, 2, 3, 4, 5 or 6, preferably i is 1 or 2,
j and k are each and independently 1, 2 or 3, and
X is O or S, preferably X is S,
wherein
in formulae (XIIIc) and (XIIIe)
one of the two nitrogen atoms is covalently attached to —CH₂— of R^{Yc}
and in formula (XIIId)
-X— is covalently attached to —CH₂— of R^{Yc}
while to the remaining nitrogen atom optionally a Z group is covalently attached, preferably the Z group comprises a chelator and optionally a linker,
and Xaa11 is a residue of formula (XIIa) 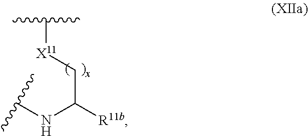
wherein
X¹¹ is —S— or —NH—,
R^{11b} is selected from the group consisting of C(═O)R^{11c}, CONH₂, CO₂H, CH₂OH and H, wherein R^{11c} is the C-terminal group C-term,
x is 1 or 2, preferably x is 1,
preferably the compound comprises a cyclic peptide of formula (Ii), 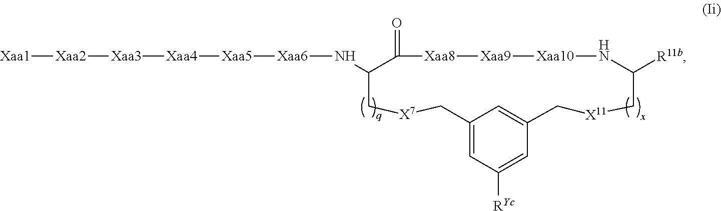
more preferably the compound comprises a cyclic peptide of formula (Ii),
wherein
X⁷ is —S— and X¹¹ is —S—,
R^{Yc} is —CH₂—R^{Yd}, and
R^{Yd} is a structure of formulae (XIIId) or (XIIIe),
wherein R^{Yf} is H, i is 1, j is 1, and k is 1,
wherein in formula (XIIId) —X— is S and is attached to —CH₂— of R^{Yc}, and in formula (XIIIe) one of the two nitrogen atoms is attached to —CH₂— of R^{Yc}, while to the remaining nitrogen atom in either (XIIId) or (XIIIe) optionally a Z group is covalently attached, preferably the Z group comprises a chelator and optionally a linker,
q is 1, and
x is 1,
most preferably the compound comprises a cyclic peptide of formula (Ii), wherein R^{Yd} is a structure of formula (XIIId), wherein —X— is S and R^{f} is H, and i is 1,
(c)
Xaa7 is a residue of formula (VIIIa) 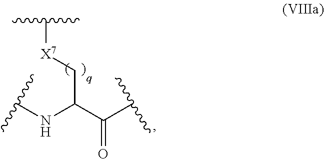
wherein
X⁷ is —S— or —NH—,
q is 1 or 2, preferably q is 1,
Yc is a structure of formula (XIIIb) 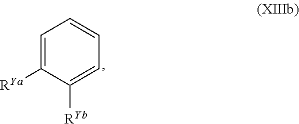
wherein
R^{Ya} is —CH₂—,
R^{Yb} is —CH₂—,
and Xaa11 is a residue of formula (XIIa) 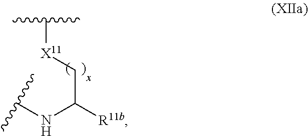
wherein
X¹¹ is —S— or —NH—,
R^{11b} is selected from the group consisting of C(═O)R^{11c}, CONH₂, CO₂H, CH₂OH and H, wherein R^{11c} is the C-terminal group C-term,
x is 1 or 2, preferably x is 1,
preferably the compound comprises a cyclic peptide of formula (Id), 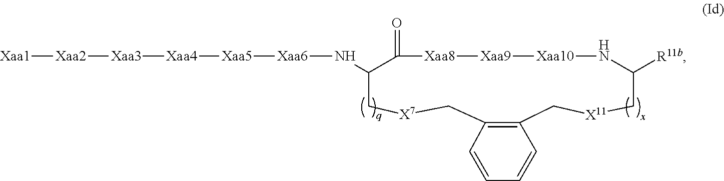
more preferably the compound comprises a cyclic peptide of formula (Id),
wherein X⁷ is —S—, X¹¹ is —S—, q is 1, and x is 1,
(d)
Xaa7 is a residue of formula (VIIIb) 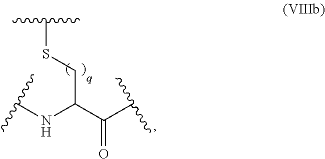
wherein
q is 1 or 2, preferably q is 1,
Yc is a structure of formula (XIIIg) 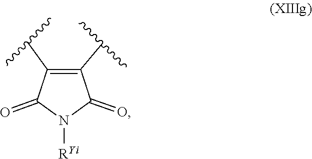
wherein
R^{Yi} is selected from the group consisting of H and (C₁-C₆)alkyl,
and Xaa11 is a residue of formula (XIIb) 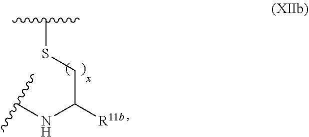
wherein
R^{11b} is selected from the group consisting of C(═O)R^{11c}, CONH₂, CO₂H, CH₂OH and H, wherein R^{11c} is the C-terminal group C-term,
x is 1 or 2, preferably x is 1,
preferably the compound comprises a cyclic peptide of formula (Ig), 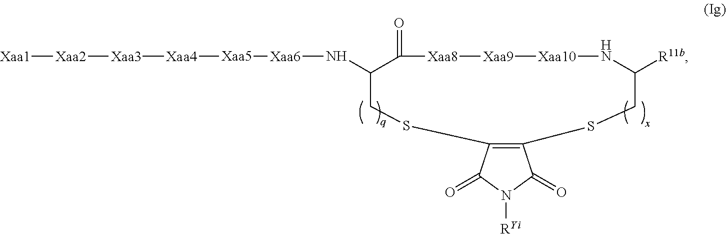
more preferably the compound comprises a cyclic peptide of formula (Ig),
wherein q is 1, and x is 1 or 2,
(e)
Yc is a structure of formula (XIIIf) 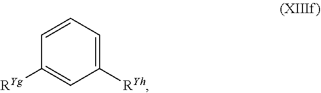
wherein
R^{Yg} is —C(═O)—,
R^{Yh} is —CH₂—,
Xaa7 is a residue of formula (VIIIa) 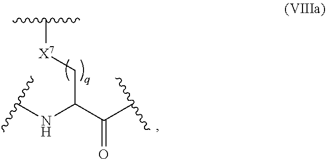
wherein
X⁷ is —S— or —NH— and is covalently attached to R^{Yh},
q is 1 or 2, preferably q is 1,
and Xaa11 is a residue of formula (XIIa) 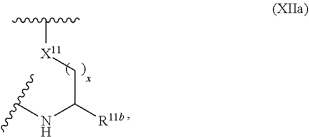
wherein
X¹¹ is —NH— and is covalently attached to R^{Yg},
R^{11b} is selected from the group consisting of C(═O)R^{11c}, CONH₂, CO₂H, CH₂OH and H, wherein R^{11c} is the C-terminal group C-term,
x is 1 or 2, preferably x is 1,
preferably the compound comprises a cyclic peptide of formula (If), 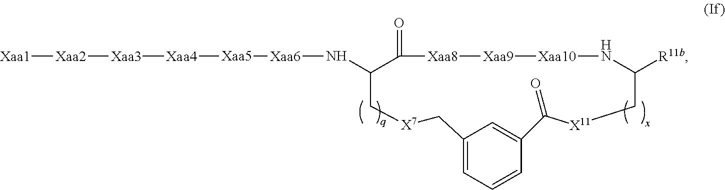
more preferably the compound comprises a cyclic peptide of formula (If),
wherein X⁷ is —S—, q is 1, and x is 1,
(f)
Yc is a structure of formula (XIIIf) 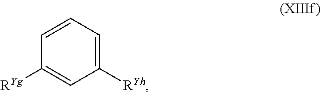
wherein
R^{Yg} is —C(═O)—,
R^{Yh} is —CH₂—,
Xaa7 is a residue of formula (VIIIa) 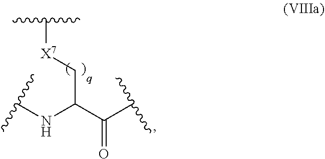
wherein
X⁷ is —NH— and is covalently attached to R^{Y9},
q is 1 or 2, preferably q is 1,
and Xaa11 is a residue of formula (XIIa) 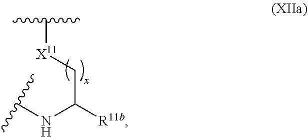
wherein
X¹¹ is —S— or —NH— and is covalently attached to R^{Yh},
R^{11b} is selected from the group consisting of C(═O)R^{11c}, CONH₂, CO₂H, CH₂OH and H, wherein R^{11c} is the C-terminal group C-term,
x is 1 or 2, preferably x is 1,
preferably the compound comprises a cyclic peptide of formula (Ie), 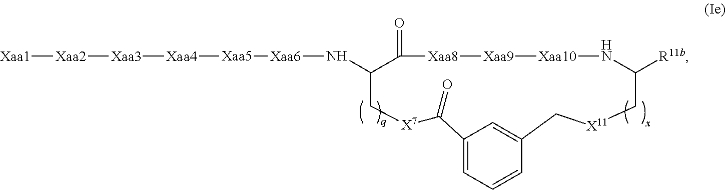
more preferably the compound comprises a cyclic peptide of formula (Ie),
wherein X¹¹ is —S—, q is 1, and x is 1, or
(g)
the compound comprises a cyclic peptide of formula (Ib), wherein
Xaa7 is a residue of formula (VIIIc) 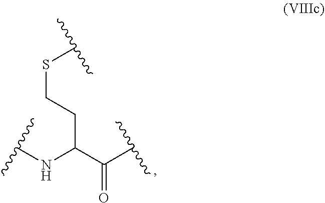 [SMILES: *NC(CCS*)C(*)=O]
and Xaa11 is a residue of formula (XIIc) 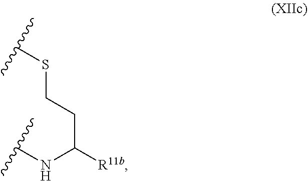
wherein
R^{11b} is selected from the group consisting of C(═O)R^{11c}, CONH₂, CO₂H, CH₂OH and H, wherein R^{11c} is the C-terminal group C-term,
preferably the compound comprises a cyclic peptide of formula (Ih),

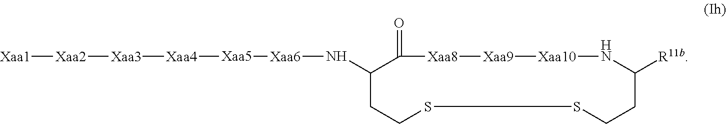

4. The compound of claim 1, wherein Xaa1 is a residue of an α-amino acid comprising a side chain, wherein the side chain comprises an acidic or polar group within the side chain or a residue of an aliphatic carboxylic acid comprising an additional acidic or polar group at the ω-position, of formula (IId) or (IIe), preferably Xaa1 is a residue of an amino acid formula (IId), 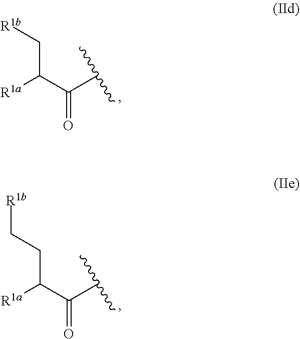
wherein
R^{1a} is selected from the group consisting of —NH—, H or —N(CH₃)—,
R^{1b} is selected from the group consisting of CO₂H, CONH₂, OH, SO₃H and OPO₃H₂, under the proviso that R^{1a} is —NH— or —N(CH₃)—, the N-terminal modification group A is covalently attached to the nitrogen atom of R^{1a},
preferably Xaa1 is a residue an α-L-amino acid selected from the group consisting of Asp, Asn, Glu, Gln, Hse, Cya, and Ser, or Xaa1 is a residue of a dicarboxylic acid residue selected from the group consisting of Succinyl and Glutar,
more preferably Xaa1 is a residue of an α-L-amino acid selected from the group consisting of Asp, Asn, Glu, and Gln,
most preferably Xaa1 is an Asp residue.

5. The compound of claim 1, wherein Xaa2 is a residue of an α-L-amino acid of formula (IIId), 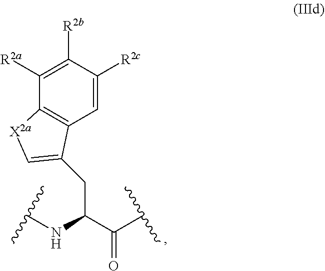
wherein
X^{2a} is NH or S, preferably is NH,
R^{2a} is H or F,
R^{2b} is selected from the group consisting of H, F and Cl,
R^{2c} is selected from the group consisting of H, F, Cl, Br and OH,
preferably Xaa2 is a residue an α-L-amino acid selected from the group consisting of Trp, Hyw, 5Fw, 6Clw, 7Fw, 5Clw, 5Brw and 6Fw,
more preferably Xaa2 is a residue an α-L-amino acid selected from the group consisting of Trp, Hyw, 5Fw and 7Fw,
most preferably Xaa2 is a Trp residue.

6. The compound of claim 1, wherein Xaa3 is a residue of an amino acid of formula (IVe) or (IVf), 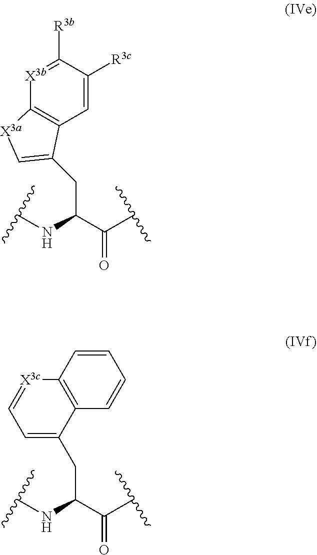
wherein
R^{3b} is H or F,
R^{3c} is selected from the group consisting of Cl, Br, H and F,
X^{3a} is NH or S,
X^{3b} is selected from the group consisting of CH, CF and N, and
X^{3c} is CH or N, preferably X^{3c} is CH,
preferably Xaa3 is a residue of an α-L-amino acid selected from the group consisting of 5Clw, 5Brw, Trp, 1Ni, Bta, 5Fw, 6Fw and 7Fw,
more preferably Xaa3 is a residue of an α-L-amino acid selected from the group consisting of 5Clw and 5Brw, and
most preferably Xaa3 is a 5Clw residue.

7. The compound of claim 1, wherein
(a)
Xaa4 is a residue of an amino acid of formula (Va) 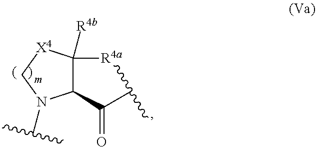
wherein
m is 1 or 2, preferably m is 1,
R^{4a} is selected from the group consisting of H, OH and methyl,
R^{4b} is either H or methyl, preferably R^{4b} is methyl if R^{4a} is methyl,
X⁴ is selected from the group consisting of CHR^{4c}, CH₂, S and O,
wherein R^{4c} is selected from the group consisting of NHR^{4d}, NH₂, H, OH, methyl and F,
wherein R^{4d} is Ac or is a Z group, preferably if the Z group is a chelator optionally comprising a linker, more preferably the chelator is DOTA, and if the Z group is a bio-distribution modifier optionally comprising a linker, more preferably the bio-distribution modifier is AGLU and the optional linker preferably is Glutar
preferably Xaa4 is a residue of an α-L-amino acid selected from the group consisting of Tap, Hyp, Pro, 4Ap, 4Ap(Ac), Tap(DOTA), Tap(AGLU-Gutar), 4Tfp and H3p,
more preferably Xaa4 is a residue of an α-L-amino acid selected from the group consisting of Tap, Hyp, Pro and 4Ap, and
most preferably Xaa4 is a Tap residue,
or
(b)
Xaa4 is a residue of an N-alkylated amino acid of formula (Vf) 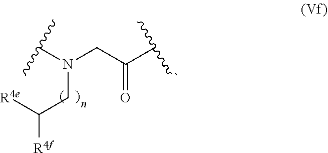
wherein
n is 0, 1 or 3,
R^{4e} is selected from the group consisting of H and phenyl, if n=0,
or is NH₂, if n=3,
or is selected from the group consisting of methyl, CO₂H, and C(═O)R^{4h}, if n=1, wherein R^{4h} is a Z group, preferably the Z group is a bio-distribution modifier optionally comprising a linker, wherein the bio-distribution modifier more preferably is AGLU, and
R^{4f} is selected from the group consisting of H and methyl,
preferably Xaa4 is a residue of an N-alkylated amino acid selected from the group consisting of Nmg, Nlys, Nleu, Nphe, Nglu and Nglu(AGLU)
or
(c)
Xaa4 is an Oic residue.

8. The compound of claim 1, wherein Xaa5 is a residue of an α-amino acid optionally comprising a Z group, wherein the α-nitrogen atom of the α-amino acid is optionally substituted by a methyl group,
preferably Xaa5 is a residue of an α-L-amino acid selected from the group consisting of Glu, Gln, Asp, Asn, Ser, Hse, Trp, Lys, Arg, His, Hly, and Har,
more preferably Xaa5 is an α-L-amino acid selected from the group consisting of Glu, Gln, Asp, Asn, Ser, and Hse,
most preferably Xaa5 is a Glu residue.

9. The compound of claim 1, wherein Xaa6 is a residue of an amino acid of formula (VIIa) 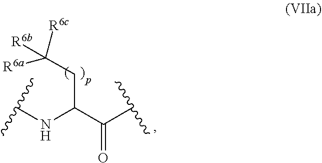
wherein
p is 1 or 0, preferably p is 1,
R^{6a} is (C₁-C₂)alkyl,
R^{6b} is methyl, and
R^{6c} is H if p is 0, and is H or methyl, if p is 1,
preferably Xaa6 is a residue of an α-amino acid selected from the group consisting of leu, Leu, Npg, Val, Ile and Hle,
more preferably Xaa6 is a residue of an α-amino acid selected from the group consisting of leu, Leu, Npg, Val and Ile, and
most preferably Xaa6 is a leu residue.

10. The compound of claim 1, wherein
(a)
Xaa8 is a residue of an amino acid of formula (IXa), 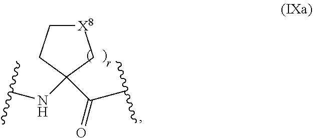
wherein
r is 2 or 1, preferably r is 2,
X⁸ is selected from the group consisting of NR^{8a}, O, NH, and CH₂, wherein R^{8a} is a Z group or R^{8a} is acetyl, wherein if the Z group is a chelator optionally comprising a linker linker, preferably the chelator is DOTA, or if the Z-group is a bio-distribution modifier optionally comprising a linker linker, preferably the bio-distribution modifier is AGLU and the linker preferably is Glutar, or if the Z group is a combination of a chelator optionally comprising a linker, a bio-distribution modifier optionally comprising a linker, and an amino acid residue linking the chelator optionally comprising a linker and the bio-distribution modifier optionally comprising a linker, preferably the chelator is DOTA and the bio-distribution modifier preferably is AGLU and the amino acid residue linking the chelator and the bio-distribution modifier preferably is a glu residue,
preferably Xaa8 is a residue an α-amino acid selected from the group consisting of Apc(R^{8a}), Ape, Thp, Egz, and Eca, wherein R^{8a} is a Z group, wherein if the Z group is a chelator optionally comprising a linker, preferably the chelator is DOTA, or if the Z-group is a bio-distribution modifier optionally comprising a linker, preferably the bio-distribution modifier is AGLU and the linker preferably is Glutar, or if the Z group is a combination of a chelator optionally comprising a linker, a bio-distribution modifier optionally comprising a linker, and an amino acid residue linking the chelator optionally comprising a linker and the bio-distribution modifier optionally comprising a linker, preferably the chelator is DOTA and the bio-distribution modifier preferably is AGLU and the amino acid residue linking the chelator and the bio-distribution modifier preferably is glu, or R^{8a} is acetyl,
more preferably Xaa8 is a residue of an α-amino acid residue selected from the group consisting of Apc(R^{8a}), Apc, and Thp, wherein R^{8a} is a Z group, wherein if the Z group is a chelator optionally comprising a linker, preferably the chelator is DOTA, or if the Z-group is a bio-distribution modifier optionally comprising a linker, preferably the bio-distribution modifier is AGLU and the linker preferably is Glutar, or R^{8a} is acetyl and
most preferably Xaa8 is Apc(R^{8a}), wherein R^{8a} is a Z group, wherein the Z group is a chelator optionally comprising a linker, wherein the chelator
a) preferably is selected from the group consisting of DOTA, DOTAGA, DOTAM, DOTP, NOTA, NODAGA, PSC, NODA-MPAA, HBED, TETA, CB-TE2A, DTPA, CHX-A″-DTPA, DFO, Macropa, HOPO, TRAP, THP, DATA, NOPO, NOTP, PCTA, sarcophagine, FSC, NETA, NE3TA, H4octapa, pycup, HYNIC, NxS4-x (N4, N2S₂, N3S), ^{99m}Tc(CO)3-chelators and their analogs,
b) more preferably is selected from the group consisting of DOTA, DOTAGA, DOTAM, NOTA, NODAGA, PSC, NODA-MPAA, NOPO, HBED, DTPA, CHX-A″-DTPA, CB-TE2A, Macropa, PCTA, N4, and analogs thereof,
and
c) most preferably is selected from the group consisting of DOTA, DOTAGA, DOTAM, NOTA, NODAGA, PSC, NOPO, Macropa, PCTA and analogs thereof,
(b) Xaa8 is an Aic residue,
(c) Xaa8 is a residue of an α-amino acid selected from the group consisting of Lys, Lys(R^{8e}), lys(R^{8e}), Amk(R^{8e}), Dab(R^{8e}) and dab(R^{8e}), wherein R^{8e} is a Z group, preferably the Z group is a chelator optionally comprising a linker, or
(d) Xaa8 is a residue of an α-amino acid residue selected from the group consisting of Aib, Deg, Ams, ala, Ala, Gln, Thr, Trp, Phe and Glu.

11. The compound of claim 1, wherein Xaa9 is a residue of an amino acid of formula (Xa) 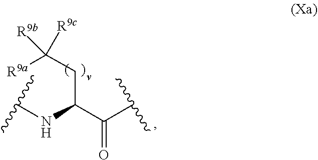
wherein
R^{9a} is selected from the group consisting of (C₁-C₄)alkyl, COOH, CONH₂, OH, NH₂, NHC(═NH)NH₂, an aryl ring and a heteroaryl ring, preferably the aryl ring is phenyl and the heteroaryl ring is 3-indoyl, and cyclo-hexyl,
R^{9b} is methyl or H,
R^{9c} is H or methyl, and
v is 0, 1, 2, preferably v is 1,
preferably Xaa9 is a residue of an α-L-amino acid selected from the group consisting of Leu, Hle, Ile, Arg, Trp, Glu, Phe, Ser, Cha, Npg and Tle,
more preferably Xaa9 is a residue of an α-L-amino acid selected from the group consisting of Leu, Hle, Ile, Arg, Trp, Glu, Phe and Ser, and
most preferably Xaa9 is a Leu residue.

12. The compound of claim 1, wherein Xaa10 is a residue of an amino acid of formula (XIa) 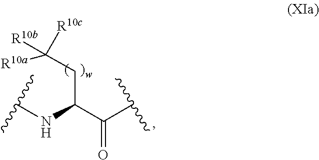
wherein
R^{10a} is selected from the group consisting of (C₁-C₂)alkyl and phenyl, preferably R^{10a} is (C₁-C₂)alkyl, more preferably (C₁-C₂)alkyl is ethyl,
R^{10b} is selected from the group consisting of methyl and H, preferably R^{10b} is methyl,
R^{10c} is selected from the group consisting of H and methyl, R^{10C} is H, if w is 1, preferably R^{10c} is H, and
w is 0 or 1, preferably w is 0,
preferably Xaa10 is a residue of an α-L-amino acid selected from the group consisting of Ile, Leu, Tle, Val, and Phe,
more preferably Xaa10 is a residue of α-L-amino acid selected from the group consisting of Ile, Leu and Tle,
most preferably Xaa10 is an Ile residue.

13. The compound of claim 1, wherein the N-terminal modification group A
a) a Z group, wherein a linker is optionally interspersed between the Z group and Xaa1,
wherein
the Z group is a chelator optionally comprising a linker,
or the Z group is a bio-distribution modifier optionally comprising a linker,
or the Z group is a combination of
a chelator optionally comprising a linker,
a bio-distribution modifier optionally comprising a linker a linker,
and an amino acid residue linking the chelator optionally comprising a linker and the bio-distribution modifier optionally comprising a linker,
b) a blocking group AblN, wherein the blocking group AblN is selected from the group consisting of acetyl and a structure of formula (AblN-Ia), 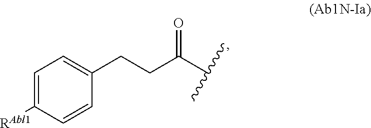
wherein
R^{Abl1} is selected from the group consisting of H, OH and a halogen, preferably the halogen is selected from the group consisting of F and Cl, preferably R^{Abl1} is OH
preferably AblN is selected from the group consisting of Ac, 4OHPhp, 4FPhp, 4ClPhp and Php,
c) the residue of an amino acid, wherein the amino acid is an α-amino acid, wherein the residue of an amino acid
optionally comprises a Z group covalently attached to the side chain of the residue of an amino acid and optionally comprises a Z group covalently attached to the α-amino group of the residue of an amino acid,
or wherein the residue of an amino acid optionally comprises a blocking group AblN covalently attached to the α-amino group of the residue of an amino acid, preferably the blocking group AblN is acetyl,
wherein the residue of an amino acid preferably is a Tyr residue, with an optional Z group covalently attached to the α-amino group of the Tyr residue, wherein the Z group is a chelator comprising an optional linker, wherein the chelator preferably is DOTA and the linker preferably is Ttds,
d) the N-terminal dipeptide group,
wherein the residue of the first amino acid, the residue of the second amino acid or both the residue of the first and of the second amino acid optionally comprise a Z group,
wherein a blocking group AblN is optionally covalently attached to the α-amino group of the residue of the first amino acid,
and wherein a linker is optionally interspersed between the residue of the second amino acid and the residue of Xaa1,
wherein if the interspersed linker is absent, the carbonyl group of the residue of the first amino acid is covalently attached to the α-nitrogen of the residue of the second amino acid and the carbonyl group of the residue of the second amino acid is covalently attached to the nitrogen atom in R^{1a} or the α-nitrogen atom of residue Xaa1,
or wherein if the interspersed linker is present, the carbonyl group of the residue of the first amino acid is covalently attached to the α-nitrogen of the residue of the second amino acid and the carbonyl group of the residue of the second amino acid is covalently attached to a nitrogen atom of the linker and a carbonyl group of the linker is covalently attached to the nitrogen atom in R^{1a} or the α-nitrogen atom of residue Xaa1,
wherein the first amino acid residue is a Met residue and the residue of the second amino acid is selected from the group comprising Ala, Tyr and tyr,
preferably the Z group is a bio-distribution modifier optionally comprising a linker, wherein the Z group is
a) preferably is selected from the group consisting of AGLU with Glutar as linker, NTASEROL, GLUSEROL with Glutar as linker, AHOL with Glutar as linker, APOL with Glutar as linker, ABOL with Glutar as linker, APrOL with Glutar as linker, MeO2kDaPEGAc, MeO10kDaPEGAc and MeO36PEG,
b) more preferably is AGLU with Glutar as linker or NTASEROL,
and
c) most preferably is AGLU with Glutar as linker,
more preferably the Z group is a bio-distribution modifier optionally comprising a linker, wherein the bio-distribution modifier is AGLU and the linker is Glutar.

14. The compound of claim 1, wherein the C-terminal group C-term is
a) the residue of an amino acid, wherein the amino acid is an α-amino acid,
optionally comprising a Z group and optionally comprising a linker interspersed between the residue of Xaa11 and the residue of an amino acid,
wherein if the interspersed linker is absent, the carbonyl group R^{11b} of residue Xaa11 is covalently attached to the α-nitrogen of the residue of an amino acid,
or wherein if the interspersed linker is present, the carbonyl group in R^{11b} of residue Xaa11 is covalently attached to a nitrogen atom of the linker, a carbonyl group of the linker is covalently attached to the α-nitrogen of the residue of an amino acid,
and wherein a blocking group AblC is covalently attached to the carbonyl group of the residue of an amino acid, wherein the blocking group AblC is selected from the group consisting of NH₂ and OH, wherein the blocking group AblC preferably is NH₂, or,
b) the C-terminal dipeptide group,
wherein the residue of the first amino acid is the residue of an α-amino acid and the residue of the second amino acid is the residue of an α-amino acid,
wherein the residue of the first amino acid, the residue of the second amino acid residue or both the residue of the first and of the second amino acid optionally comprise a Z group,
and wherein a linker is optionally interspersed between the residue of Xaa11 and the residue of the first amino acid,
wherein if the interspersed linker is absent, the carbonyl group R^{11b} of Xaa11 is covalently attached to the α-nitrogen of the residue of the first amino acid and the carbonyl group of the residue of the first amino acid is covalently attached to the α-nitrogen of the residue of the second amino acid,
or wherein if the interspersed linker is present, the carbonyl group R^{11b} of Xaa11 is covalently attached to a nitrogen atom of the linker, a carbonyl group of the linker is covalently attached to the α-nitrogen of the residue of the first amino acid and the carbonyl group of the residue of the first amino acid is covalently attached to the α-nitrogen of the residue of the second amino acid,
and wherein a blocking group AblC is covalently attached to the carbonyl group of the residue of the second amino acid, wherein the blocking group AblC is selected from the group consisting of NH₂ and OH, wherein the blocking group AblC preferably is NH₂, or
c) a Z group.

15. The compound of claim 13, wherein the C-terminal group C-term is the residue of an α-amino acid, wherein the α-amino acid is selected from
i) an α-amino acid comprising a side chain AaaA, wherein the side chain comprises an amino group, wherein to the amino group a Z group is covalently attached by an amide bond, wherein the Z group is a chelator optionally comprising a linker, wherein the chelator
a) preferably is selected from the group consisting of DOTA, DOTAGA, DOTAM, DOTP, NOTA, NODAGA, PSC, NODA-MPAA, HBED, TETA, CB-TE2A, DTPA, CHX-A″-DTPA, DFO, Macropa, HOPO, TRAP, THP, DATA, NOPO, NOTP, PCTA, sarcophagine, FSC, NETA, NE3TA, H4octapa, pycup, HYNIC, NxS4×(N4, N2S₂, N3S), 99mTc(CO)₃-chelators and their analogs,
b) more preferably is selected from the group consisting of DOTA, DOTAGA, DOTAM, NOTA, NODAGA, PSC, NODA-MPAA, NOPO, HBED, DTPA, CHX A″-DTPA, CB-TE2A, Macropa, PCTA, N4, and analogs thereof, or
c) most preferably is selected from the group consisting of DOTA, DOTAGA, DOTAM, NOTA, NODAGA, PSC, NOPO, Macropa, PCTA and analogs thereof,
or
ii) an α-amino acid comprising a side chain AaaB, wherein the side chain comprises an amino group, wherein to the amino group a Z group is covalently attached by an amide bond, wherein the Z group is a bio-distribution modifier optionally comprising a linker, wherein the Z group
a) preferably is selected from the group consisting of AGLU with Glutar as linker, NTASEROL, GLUSEROL with Glutar as linker, AHOL with Glutar as linker, APOL with Glutar as linker, ABOL with Glutar as linker, APrOL with Glutar as linker, MeO2kDaPEGAc, MeO10kDaPEGAc and MeO36PEG, or
b) more preferably is MeO36PEG or NTASEROL,
or
iii) an α-amino acid comprising a side chain AaaC, wherein the side chain comprises a COOH group, where to the COOH group a Z group is covalently attached by an amide bond, wherein the Z group is a bio-distribution modifier optionally comprising a linker, wherein the bio-distribution modifier
a) preferably is selected from the group consisting of AGLU, GLUSEROL, AHOL, APOL, ABOL, and APrOL, or
b) more preferably is AGLU;
wherein the blocking group AblC is NH₂,
and wherein an optional linker is interspersed between Xaa11 and the residue of the α-amino acid, and said linker preferably is APAc or Ttds.

16. The compound of claim 1, wherein the compound comprises one Z group, two Z groups, three Z groups or four Z groups.

17. The compound of claim 16, wherein the compound comprises three Z groups, wherein
one of the three Z groups is a chelator optionally comprising a linker, and the other two of the three Z groups are bio-distribution modifiers optionally comprising a linker, wherein the chelator optionally comprising a linker preferably
a) is covalently attached to or is part of substituent R^{8a}, under the proviso that residue Xaa8 is a structure of formula (XIa), (IXd) or (IXe),
b) is covalently attached to or is part of substituent R^{8e}, under the proviso that residue Xaa8 is a structure of formula (XIc),
c) is covalently attached to the residue of an α-amino acid of the C-terminal group C-term,
or
d) is part of or forms the N-terminal modification group A,
and wherein either of the two the bio-distribution modifiers optionally comprising a linker
a) is covalently attached to or is part of substituent R^{8a}, under the proviso that residue Xaa8 is a structure of formulae (XIa), (IXd) or (IXe),
b) is covalently attached to or is part of substituent R^{8e}, under the proviso that residue Xaa8 is a structure of formula (XIc),
c) is covalently attached to the residue of an α-amino acid of the C-terminal group C-term,
or
d) is part of or forms the N-terminal modification group A,
preferably the chelator optionally comprising a linker
a) is covalently attached to or is part of substituent R^{8a}, under the proviso that residue Xaa8 is a structure of formula (XIa), (IXd) or (IXe),
or
b) is covalently attached to or is part of substituent R^{8e}, under the proviso that residue Xaa8 is a structure of formula (XIc),
and wherein one of the two bio-distribution modifiers each optionally comprising a linker
is part of or forms the N-terminal modification group A,
and wherein the other of the two bio-distribution modifiers each optionally comprising a linker
is covalently attached to the residue of an α-amino acids of the C-terminal group C-term.

18. The compound of claim 1,
wherein
Xaa1 is Asp, Asn, Glu, Gln, Succinyl, Glutar or Cya,
Xaa2 is Trp, Hyw, 5Fw, 6Clw or 7Fw,
Xaa3 is 5Clw, 5Brw, 5Fw, Trp, 1Ni, Bta, 6Fw or Trp,
Xaa4 is Tap, Hyp, 4Tfp, Pro, H3p, Eaz, Oxa, Dtc, Nmg, Nleu, Nlys, Nphe or Nglu,
Xaa5 is Glu, glu, Nme, Gln, Asp, Asn or Hse,
Xaa6 is leu, Leu, Npg, Val or Ile,
Xaa7 is Cys or Hey,
Xaa8 is Apc(DOTA), Thp, Apc, Aib, Deg, Ams, Amk(DOTA), Egz, Eca, or ala,
Xaa9 is Leu, Hle, Ile, Phe, Glu, Arg, Ser or Trp,
Xaa10 is Ile, Leu, Val or Tle,
Xaa11 is Cys, Hey or AET, and
Yc is 3MeBn or 3Lut, or
wherein
Xaa1 is Asp, Glu or Succinyl,
Xaa2 is Trp or Hyw,
Xaa3 is 5Clw or 5Brw,
Xaa4 is Tap, Hyp, or Pro,
Xaa5 is Glu,
Xaa6 is leu or Leu,
Xaa7 is Cys,
Xaa8 is Apc(DOTA) or Thp,
Xaa9 is Leu,
Xaa10 is Ile
Xaa11 is Cys and
Yc is 3Lut, or
wherein
Xaa1 is Asp,
Xaa2 is Trp,
Xaa3 is 5Clw,
Xaa4 is Tap or Hyp,
Xaa5 is Glu,
Xaa6 is leu,
Xaa7 is Cys,
Xaa8 is Apc(DOTA),
Xaa9 is Leu,
Xaa10 is Ile
Xaa11 is Cys and
Yc is 3Lut.
preferably
the compound comprises three Z groups,
wherein a first of the three Z groups is a chelator,
wherein the chelator is covalently attached to Xaa8,
wherein Xaa8 is a Apc residue and wherein the chelator is DOTA,
and
wherein a second of the three Z groups is bio-distribution modifier optionally comprising a linker,
wherein the bio-distribution modifier and the linker form the N-terminal modification group A,
wherein the N-terminal modification group A is AGLU-Glutar,
and
wherein a third of the three Z groups is a bio-distribution modifier,
wherein the bio-distribution modifier is covalently attached to the residue of an α-amino acid of the C-terminal group C-term
and wherein the C-terminal group C-term is APAc-glu(AGLU)-NH₂.

19. The compound of claim 1, wherein the compound is selected from the group consisting of compound AGLU-Glutar-Asp-Trp-5Clw-Tap-Glu-leu-[Cys(2Lut)-Apc(DOTA)-Leu-Ile-Cys]-APAc-glu(AGLU)-NH₂ (GIP-001) of the following formula 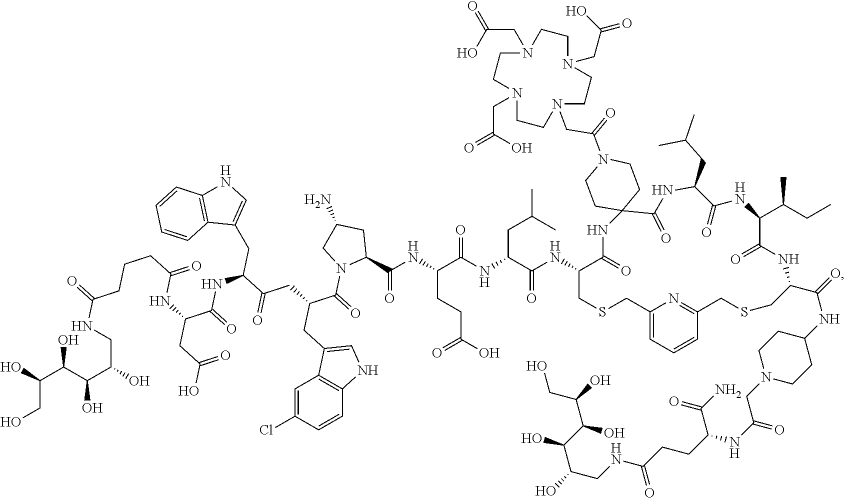 [SMILES: CC[C@H](C)[C@@H]1NC(=O)[C@H](CC(C)C)NC(=O)C2(CCN(C(=O)CN3CCN(CC(=O)O)CCN(CC(=O)O)CCN(CC(=O)O)CC3)CC2)NC(=O)[C@@H](NC(=O)[C@@H](CC(C)C)NC(=O)[C@H](CCC(=O)O)NC(=O)[C@@H]2C[C@@H](N)CN2C(=O)[C@@H](CC(=O)[C@H](Cc2c[nH]c3ccccc23)NC(=O)[C@H](CC(=O)O)NC(=O)CCCC(=O)NC[C@H](O)[C@@H](O)[C@H](O)[C@H](O)CO)Cc2c[nH]c3ccc(Cl)cc23)CSCc2cccc(n2)CSC[C@@H](C(=O)NC2CCN(CC(=O)N[C@H](CCC(=O)NC[C@H](O)[C@@H](O)[C@H](O)[C@H](O)CO)C(N)=O)CC2)NC1=O]
compound AGLU-Glutar-Asp-Trp-5Clw-Hyp-Glu-leu-[Cys(2Lut)-Apc(DOTA)-Leu-Ile-Cys]-APAc-glu(AGLU)-NH₂ (GIP-002) of the following formula 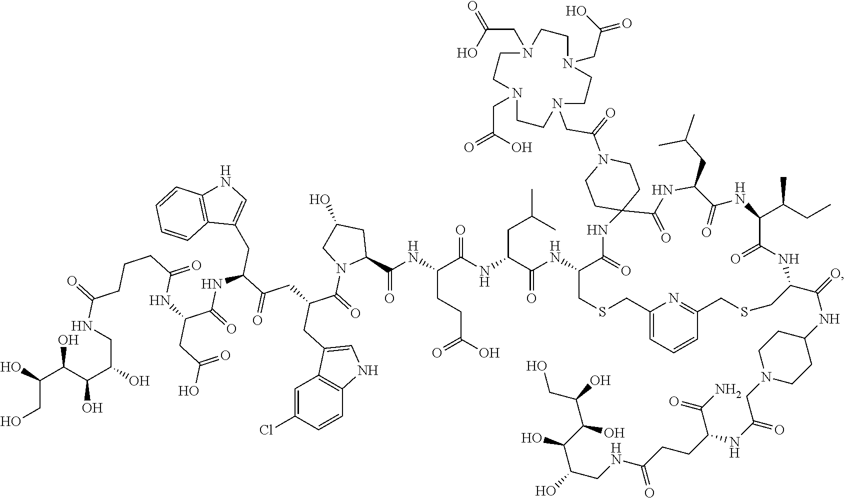 [SMILES: CC[C@H](C)[C@@H]1NC(=O)[C@H](CC(C)C)NC(=O)C2(CCN(C(=O)CN3CCN(CC(=O)O)CCN(CC(=O)O)CCN(CC(=O)O)CC3)CC2)NC(=O)[C@@H](NC(=O)[C@@H](CC(C)C)NC(=O)[C@H](CCC(=O)O)NC(=O)[C@@H]2C[C@@H](O)CN2C(=O)[C@@H](CC(=O)[C@H](Cc2c[nH]c3ccccc23)NC(=O)[C@H](CC(=O)O)NC(=O)CCCC(=O)NC[C@H](O)[C@@H](O)[C@H](O)[C@H](O)CO)Cc2c[nH]c3ccc(Cl)cc23)CSCc2cccc(n2)CSC[C@@H](C(=O)NC2CCN(CC(=O)N[C@H](CCC(=O)NC[C@H](O)[C@@H](O)[C@H](O)[C@H](O)CO)C(N)=O)CC2)NC1=O]
compound DOTA-glu(AGLU)-APAc-Asp-Trp-5Clw-Hyp-Glu-leu-[Cys(2Lut)-Apc(AGLU-Glutar)-Leu-Ile-Cys]-APAc-glu(AGLU)-NH₂ (GIP-003) of the following formula 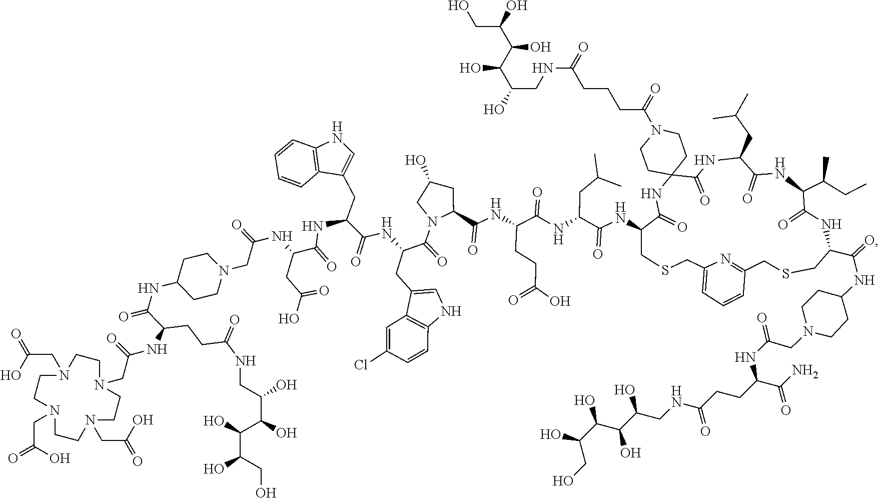 [SMILES: CC[C@H](C)[C@@H]1NC(=O)[C@H](CC(C)C)NC(=O)C2(CCN(C(=O)CCCC(=O)NC[C@H](O)[C@@H](O)[C@H](O)[C@H](O)CO)CC2)NC(=O)[C@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CC(=O)O)NC(=O)CN2CCC(NC(=O)[C@@H](CCC(=O)NC[C@H](O)[C@@H](O)[C@H](O)[C@H](O)CO)NC(=O)CN3CCN(CC(=O)O)CCN(CC(=O)O)CCN(CC(=O)O)CC3)CC2)CSCc2cccc(n2)CSC[C@@H](C(=O)NC2CCN(CC(=O)N[C@H](CCC(=O)NC[C@H](O)[C@@H](O)[C@H](O)[C@H](O)CO)C(N)=O)CC2)NC1=O]
compound AGLU-Glutar-Asp-Trp-5Clw-Hyp-Glu-leu-[Cys(2Lut)-Thp-Leu-Ile-Cys]-APAc-glu(AGLU)-Lys(DOTA)-NH₂ (GIP-004) of the following formula 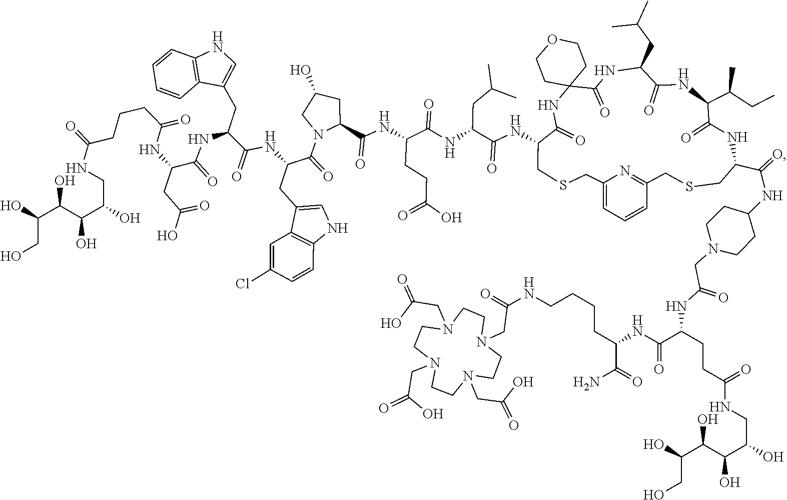 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CC(=O)O)NC(=O)CCCC(=O)NC[C@H](O)[C@@H](O)[C@H](O)[C@H](O)CO)CSCc2cccc(n2)CSC[C@@H](C(=O)NC2CCN(CC(=O)N[C@H](CCC(=O)NC[C@H](O)[C@@H](O)[C@H](O)[C@H](O)CO)C(=O)N[C@@H](CCCCNC(=O)CN3CCN(CC(=O)O)CCN(CC(=O)O)CCN(CC(=O)O)CC3)C(N)=O)CC2)NC1=O]
compound DOTA-glu(AGLU)-APAc-Asp-Trp-5Clw-Hyp-Glu-leu-[Cys(2Lut)-Thp-Leu-Ile-Cys]-APAc-glu(AGLU)-NH₂ (GIP-005) of the following formula 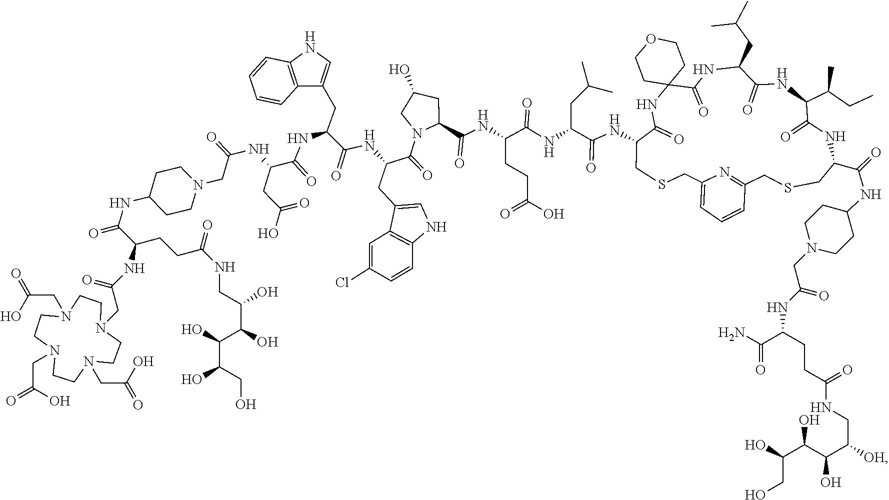 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CC(=O)O)NC(=O)CN2CCC(NC(=O)[C@@H](CCC(=O)NC[C@H](O)[C@@H](O)[C@H](O)[C@H](O)CO)NC(=O)CN3CCN(CC(=O)O)CCN(CC(=O)O)CCN(CC(=O)O)CC3)CC2)CSCc2cccc(n2)CSC[C@@H](C(=O)NC2CCN(CC(=O)N[C@H](CCC(=O)NC[C@H](O)[C@@H](O)[C@H](O)[C@H](O)CO)C(N)=O)CC2)NC1=O]
compound AGLU-Glutar-APAc-Asp-Trp-5Clw-Tap(AGLU-Glutar)-Glu-leu-[Cys(2Lut)-Apc(DOTA)-Leu-Ile-Cys]-APAc-glu(AGLU)-NH₂ (GIP-006) of the following formula 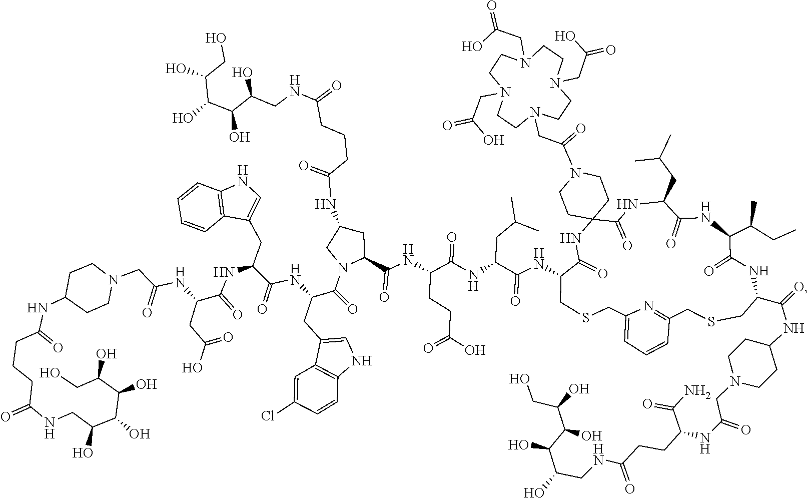 [SMILES: CC[C@H](C)[C@@H]1NC(=O)[C@H](CC(C)C)NC(=O)C2(CCN(C(=O)CN3CCN(CC(=O)O)CCN(CC(=O)O)CCN(CC(=O)O)CC3)CC2)NC(=O)[C@@H](NC(=O)[C@@H](CC(C)C)NC(=O)[C@H](CCC(=O)O)NC(=O)[C@@H]2C[C@@H](NC(=O)CCCC(=O)NC[C@H](O)[C@@H](O)[C@H](O)[C@H](O)CO)CN2C(=O)[C@H](Cc2c[nH]c3ccc(Cl)cc23)NC(=O)[C@H](Cc2c[nH]c3ccccc23)NC(=O)[C@H](CC(=O)O)NC(=O)CN2CCC(NC(=O)CCCC(=O)NC[C@H](O)[C@@H](O)[C@H](O)[C@H](O)CO)CC2)CSCc2cccc(n2)CSC[C@@H](C(=O)NC2CCN(CC(=O)N[C@H](CCC(=O)NC[C@H](O)[C@@H](O)[C@H](O)[C@H](O)CO)C(N)=O)CC2)NC1=O]
compound AGLU-Glutar-APAc-Asp-Trp-5Clw-Tap(AGLU-Glutar)-Glu-leu-[Cys(2Lut)-Apc(DOTA-glu(AGLU))-Leu-Ile-Cys]-APAc-glu(AGLU)-NH₂ (GIP-007) of the following formula 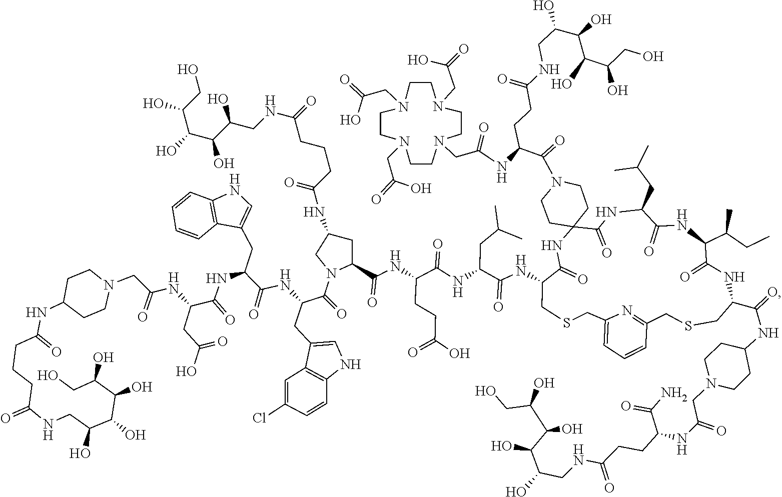
compound AGLU-Glutar-APAc-Asp-Trp-5Clw-Tap-Glu-leu-[Cys(2Lut)-Apc(DOTA-glu(AGLU))-Leu-Ile-Cys]-APAc-glu(AGLU)-NH₂ (GIP-008) of the following formula 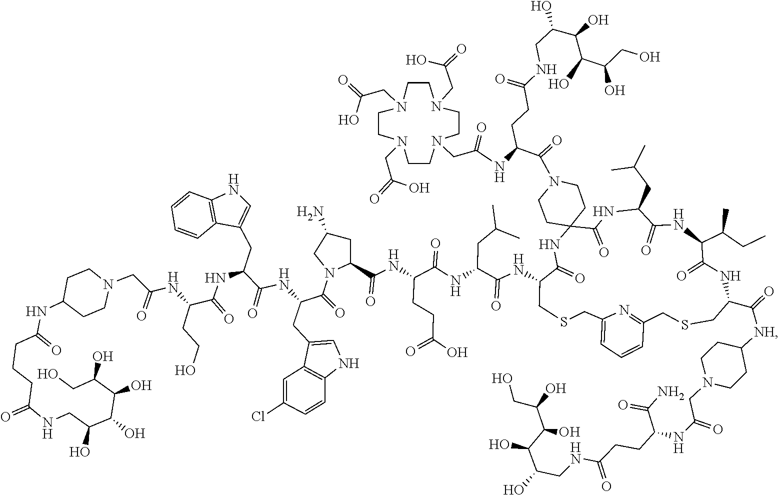 [SMILES: CC[C@H](C)[C@@H]1NC(=O)[C@H](CC(C)C)NC(=O)C2(CCN(C(=O)[C@H](CCC(=O)NC[C@H](O)[C@@H](O)[C@H](O)[C@H](O)CO)NC(=O)CN3CCN(CC(=O)O)CCN(CC(=O)O)CCN(CC(=O)O)CC3)CC2)NC(=O)[C@@H](NC(=O)[C@@H](CC(C)C)NC(=O)[C@H](CCC(=O)O)NC(=O)[C@@H]2C[C@@H](N)CN2C(=O)[C@H](Cc2c[nH]c3ccc(Cl)cc23)NC(=O)[C@H](Cc2c[nH]c3ccccc23)NC(=O)[C@H](CCO)NC(=O)CN2CCC(NC(=O)CCCC(=O)NC[C@H](O)[C@@H](O)[C@H](O)[C@H](O)CO)CC2)CSCc2cccc(n2)CSC[C@@H](C(=O)NC2CCN(CC(=O)N[C@H](CCC(=O)NC[C@H](O)[C@@H](O)[C@H](O)[C@H](O)CO)C(N)=O)CC2)NC1=O]
compound DOTA-glu(AGLU)-O2Oc-Asp-Trp-5Clw-Tap-Glu-leu-[Cys(2Lut)-Apc(AGLU-Glutar)-Leu-Ile-Cys]-APAc-glu(AGLU)-NH₂ (GIP-009) of the following formula 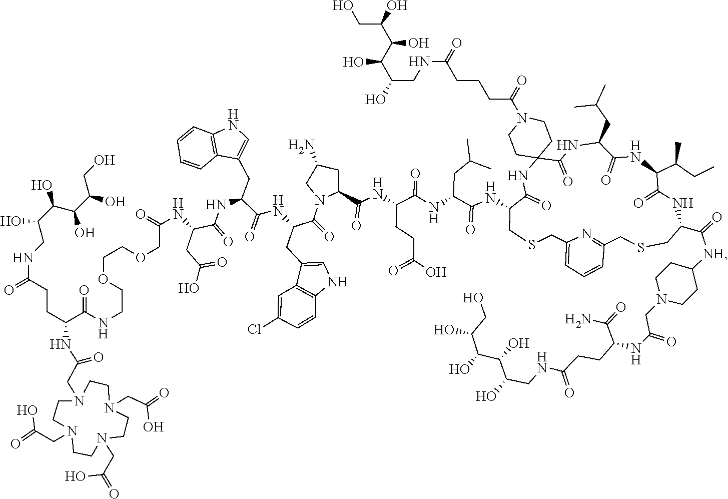 [SMILES: CC[C@H](C)[C@@H]1NC(=O)[C@H](CC(C)C)NC(=O)C2(CCN(C(=O)CCCC(=O)NC[C@H](O)[C@@H](O)[C@H](O)[C@H](O)CO)CC2)NC(=O)[C@@H](NC(=O)[C@@H](CC(C)C)NC(=O)[C@H](CCC(=O)O)NC(=O)[C@@H]2C[C@@H](N)CN2C(=O)[C@H](Cc2c[nH]c3ccc(Cl)cc23)NC(=O)[C@H](Cc2c[nH]c3ccccc23)NC(=O)[C@H](CC(=O)O)NC(=O)COCCOCCNC(=O)[C@@H](CCC(=O)NC[C@H](O)[C@@H](O)[C@H](O)[C@H](O)CO)NC(=O)CN2CCN(CC(=O)O)CCN(CC(=O)O)CCN(CC(=O)O)CC2)CSCc2cccc(n2)CSC[C@@H](C(=O)NC2CCN(CC(=O)N[C@H](CCC(=O)NC[C@H](O)[C@@H](O)[C@H](O)[C@H](O)CO)C(N)=O)CC2)NC1=O]
compound AGLU-Glutar-Asp-Trp-5Clw-Hyp-Glu-leu-[Cys(2Lut)-Apc(AGLU-Glutar)-Leu-Ile-Cys]-APAc-Lys(DOTA)-NH₂ (GIP-010) of the following formula 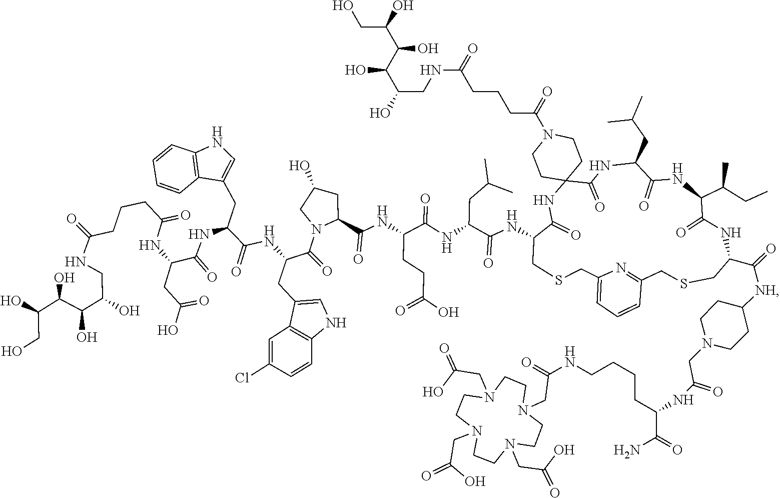 [SMILES: CC[C@H](C)[C@@H]1NC(=O)[C@H](CC(C)C)NC(=O)C2(CCN(C(=O)CCCC(=O)NC[C@H](O)[C@@H](O)[C@H](O)[C@H](O)CO)CC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CC(=O)O)NC(=O)CCCC(=O)NC[C@H](O)[C@@H](O)[C@H](O)[C@H](O)CO)CSCc2cccc(n2)CSC[C@@H](C(=O)NC2CCN(CC(=O)N[C@@H](CCCCNC(=O)CN3CCN(CC(=O)O)CCN(CC(=O)O)CCN(CC(=O)O)CC3)C(N)=O)CC2)NC1=O]
compound AGLU-Glutar-APAc-Asp-Trp-5Clw-Hyp-Glu-leu-[Cys(2Lut)-Apc(DOTA)-Leu-Ile-Cys]-APAc-glu(AGLU)-NH₂ (GIP-011) of the following formula 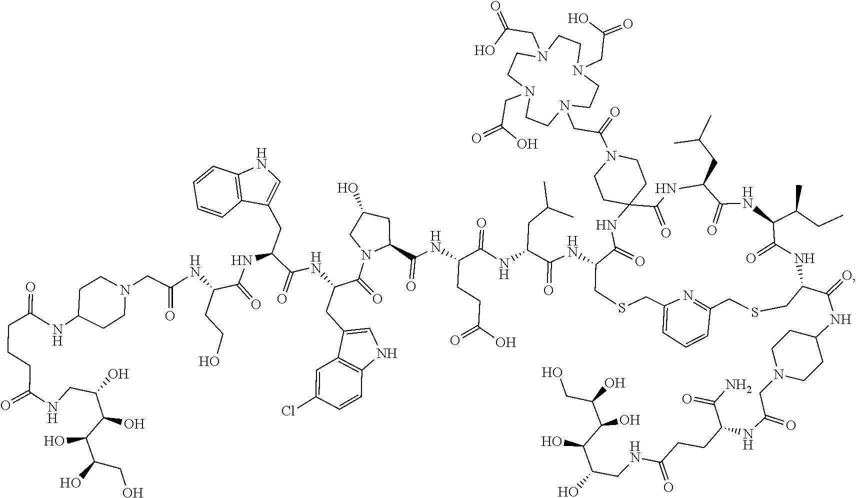 [SMILES: CC[C@H](C)[C@@H]1NC(=O)[C@H](CC(C)C)NC(=O)C2(CCN(C(=O)CN3CCN(CC(=O)O)CCN(CC(=O)O)CCN(CC(=O)O)CC3)CC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CCO)NC(=O)CN2CCC(NC(=O)CCCC(=O)NC[C@H](O)[C@@H](O)[C@H](O)[C@H](O)CO)CC2)CSCc2cccc(n2)CSC[C@@H](C(=O)NC2CCN(CC(=O)N[C@H](CCC(=O)NC[C@H](O)[C@@H](O)[C@H](O)[C@H](O)CO)C(N)=O)CC2)NC1=O]
compound DOTA-APAc-Asp-Trp-5Clw-Hyp-Glu-leu-[Cys(2Lut)-Apc(AGLU-Glutar)-Leu-Ile-Cys]-APAc-glu(AGLU)-NH₂ (GIP-012) of the following formula 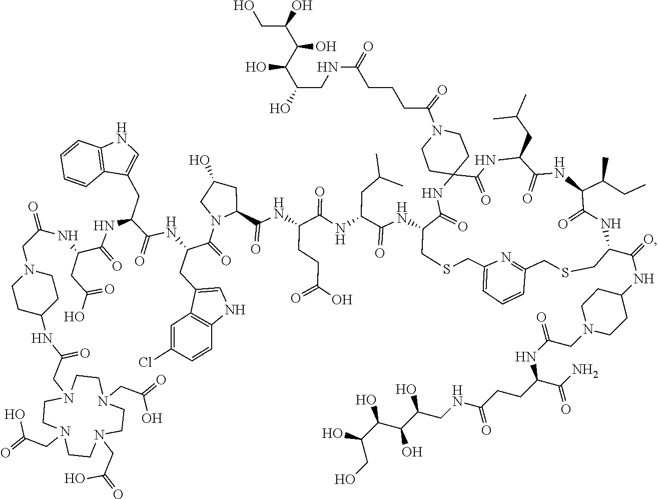 [SMILES: CC[C@H](C)[C@@H]1NC(=O)[C@H](CC(C)C)NC(=O)C2(CCN(C(=O)CCCC(=O)NC[C@H](O)[C@@H](O)[C@H](O)[C@H](O)CO)CC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CC(=O)O)NC(=O)CN2CCC(NC(=O)CN3CCN(CC(=O)O)CCN(CC(=O)O)CCN(CC(=O)O)CC3)CC2)CSCc2cccc(n2)CSC[C@@H](C(=O)NC2CCN(CC(=O)N[C@H](CCC(=O)NC[C@H](O)[C@@H](O)[C@H](O)[C@H](O)CO)C(N)=O)CC2)NC1=O]
compound AGLU-Glutar-APAc-Asp-Trp-5Clw-Tap-Glu-leu-[Cys(2Lut)-Apc(DOTA)-Leu-Ile-Cys]-APAc-glu(AGLU)-NH₂ (GIP-013) of the following formula 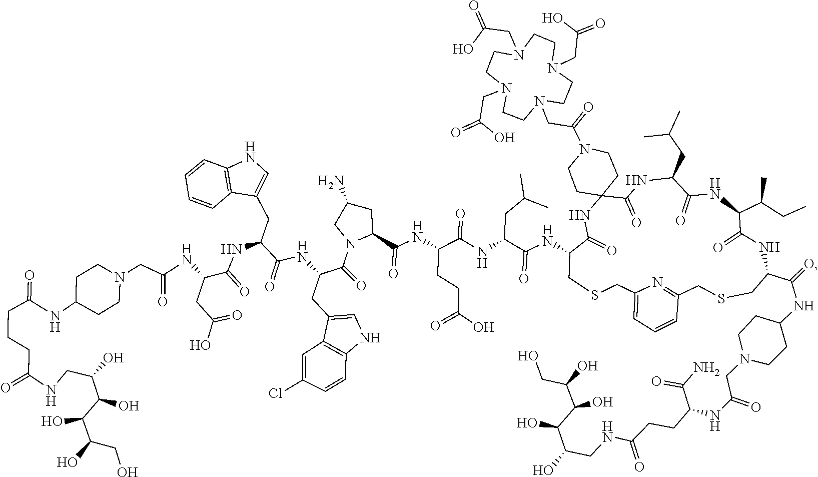 [SMILES: CC[C@H](C)[C@@H]1NC(=O)[C@H](CC(C)C)NC(=O)C2(CCN(C(=O)CN3CCN(CC(=O)O)CCN(CC(=O)O)CCN(CC(=O)O)CC3)CC2)NC(=O)[C@@H](NC(=O)[C@@H](CC(C)C)NC(=O)[C@H](CCC(=O)O)NC(=O)[C@@H]2C[C@@H](N)CN2C(=O)[C@H](Cc2c[nH]c3ccc(Cl)cc23)NC(=O)[C@H](Cc2c[nH]c3ccccc23)NC(=O)[C@H](CC(=O)O)NC(=O)CN2CCC(NC(=O)CCCC(=O)NC[C@H](O)[C@@H](O)[C@H](O)[C@H](O)CO)CC2)CSCc2cccc(n2)CSC[C@@H](C(=O)NC2CCN(CC(=O)N[C@H](CCC(=O)NC[C@H](O)[C@@H](O)[C@H](O)[C@H](O)CO)C(N)=O)CC2)NC1=O]
compound AGLU-Glutar-APAc-Asp-Trp-5Brw-Tap-Glu-leu-[Cys(2Lut)-Apc(DOTA)-Leu-Ile-Cys]-APAc-glu(AGLU)-NH₂ (GIP-014) of the following formula 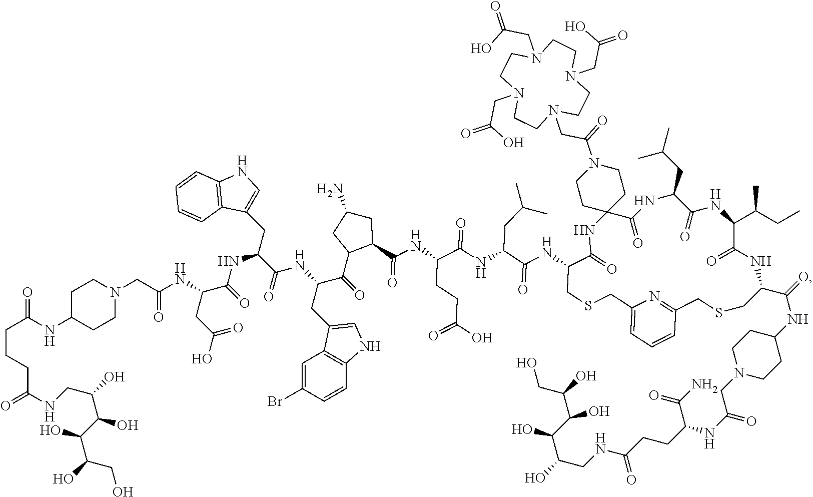 [SMILES: CC[C@H](C)[C@@H]1NC(=O)[C@H](CC(C)C)NC(=O)C2(CCN(C(=O)CN3CCN(CC(=O)O)CCN(CC(=O)O)CCN(CC(=O)O)CC3)CC2)NC(=O)[C@@H](NC(=O)[C@@H](CC(C)C)NC(=O)[C@H](CCC(=O)O)NC(=O)[C@@H]2C[C@@H](N)CC2C(=O)[C@H](Cc2c[nH]c3ccc(Br)cc23)NC(=O)[C@H](Cc2c[nH]c3ccccc23)NC(=O)[C@H](CC(=O)O)NC(=O)CN2CCC(NC(=O)CCCC(=O)NC[C@H](O)[C@@H](O)[C@H](O)[C@H](O)CO)CC2)CSCc2cccc(n2)CSC[C@@H](C(=O)NC2CCN(CC(=O)N[C@H](CCC(=O)NC[C@H](O)[C@@H](O)[C@H](O)[C@H](O)CO)C(N)=O)CC2)NC1=O]
compound AGLU-Glutar-Asp-Trp-5Brw-Tap-Glu-leu-[Cys(2Lut)-Apc(DOTA)-Leu-Ile-Cys]-APAc-glu(AGLU)-NH₂ (GIP-015) of the following formula 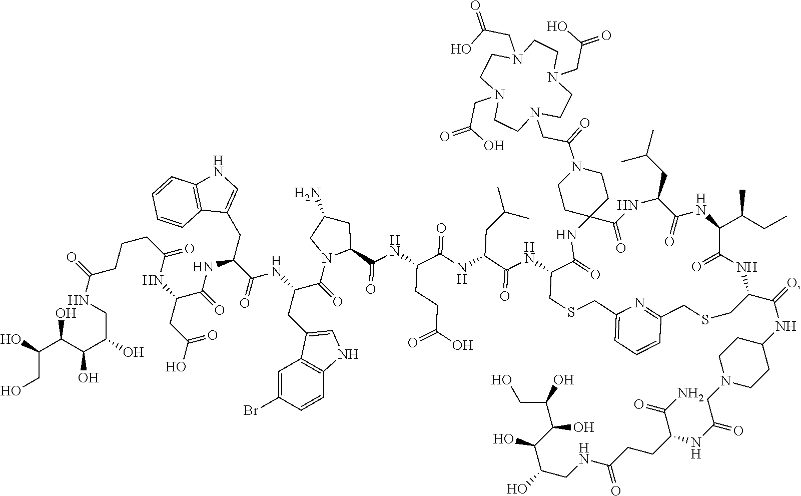 [SMILES: CC[C@H](C)[C@@H]1NC(=O)[C@H](CC(C)C)NC(=O)C2(CCN(C(=O)CN3CCN(CC(=O)O)CCN(CC(=O)O)CCN(CC(=O)O)CC3)CC2)NC(=O)[C@@H](NC(=O)[C@@H](CC(C)C)NC(=O)[C@H](CCC(=O)O)NC(=O)[C@@H]2C[C@@H](N)CN2C(=O)[C@H](Cc2c[nH]c3ccc(Br)cc23)NC(=O)[C@H](Cc2c[nH]c3ccccc23)NC(=O)[C@H](CC(=O)O)NC(=O)CCCC(=O)NC[C@H](O)[C@@H](O)[C@H](O)[C@H](O)CO)CSCc2cccc(n2)CSC[C@@H](C(=O)NC2CCN(CC(=O)N[C@H](CCC(=O)NC[C@H](O)[C@@H](O)[C@H](O)[C@H](O)CO)C(N)=O)CC2)NC1=O]
compound AGLU-Glutar-Hse-Trp-5Brw-Tap-Glu-leu-[Cys(2Lut)-Apc(DOTA)-Leu-Ile-Cys]-APAc-glu(AGLU)-NH₂ (GIP-016) of the following formula 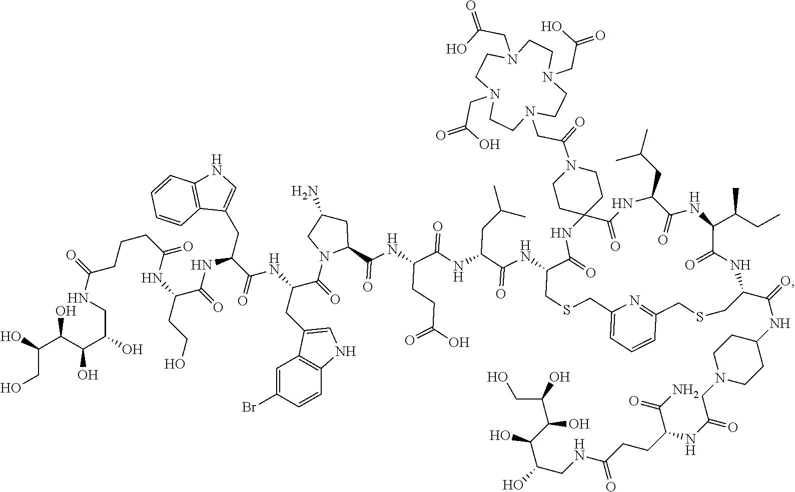 [SMILES: CC[C@H](C)[C@@H]1NC(=O)[C@H](CC(C)C)NC(=O)C2(CCN(C(=O)CN3CCN(CC(=O)O)CCN(CC(=O)O)CCN(CC(=O)O)CC3)CC2)NC(=O)[C@@H](NC(=O)[C@@H](CC(C)C)NC(=O)[C@H](CCC(=O)O)NC(=O)[C@@H]2C[C@@H](N)CN2C(=O)[C@H](Cc2c[nH]c3ccc(Br)cc23)NC(=O)[C@H](Cc2c[nH]c3ccccc23)NC(=O)[C@H](CCO)NC(=O)CCCC(=O)NC[C@H](O)[C@@H](O)[C@H](O)[C@H](O)CO)CSCc2cccc(n2)CSC[C@@H](C(=O)NC2CCN(CC(=O)N[C@H](CCC(=O)NC[C@H](O)[C@@H](O)[C@H](O)[C@H](O)CO)C(N)=O)CC2)NC1=O]
compound DOTA-glu(AGLU)-Asp-Trp-5Clw-Tap-Glu-leu-[Cys(2Lut)-Thp-Leu-Ile-Cys]-APAc-glu(AGLU)-NH₂ (GIP-017) of the following formula 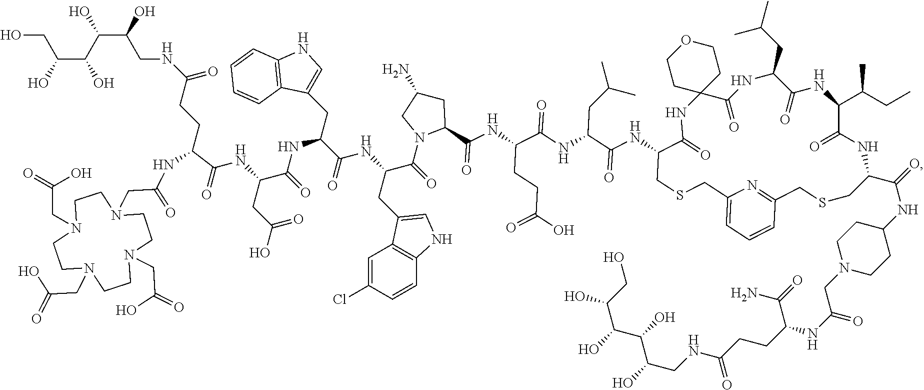 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](N)CN2C(=O)[C@H](Cc2c[nH]c3ccc(Cl)cc23)NC(=O)[C@H](Cc2c[nH]c3ccccc23)NC(=O)[C@H](CC(=O)O)NC(=O)[C@@H](CCC(=O)NC[C@H](O)[C@@H](O)[C@H](O)[C@H](O)CO)NC(=O)CN2CCN(CC(=O)O)CCN(CC(=O)O)CCN(CC(=O)O)CC2)CSCc2cccc(n2)CSC[C@@H](C(=O)NC2CCN(CC(=O)N[C@H](CCC(=O)NC[C@H](O)[C@@H](O)[C@H](O)[C@H](O)CO)C(N)=O)CC2)NC1=O]
compound DOTA-O2Oc-glu(AGLU)-Asp-Trp-5Clw-Tap-Glu-leu-[Cys(2Lut)-Thp-Leu-Ile-Cys]-APAc-glu(AGLU)-NH₂ (GIP-018) of the following formula 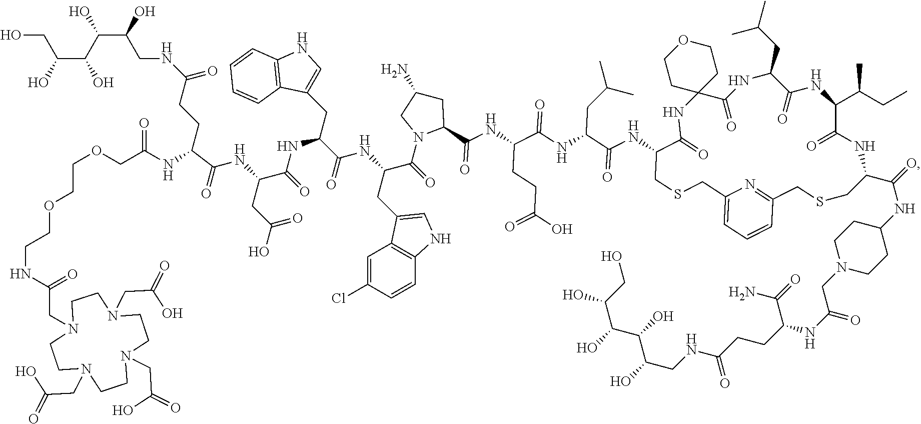 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](N)CN2C(=O)[C@H](Cc2c[nH]c3ccc(Cl)cc23)NC(=O)[C@H](Cc2c[nH]c3ccccc23)NC(=O)[C@H](CC(=O)O)NC(=O)[C@@H](CCC(=O)NC[C@H](O)[C@@H](O)[C@H](O)[C@H](O)CO)NC(=O)COCCOCCNC(=O)CN2CCN(CC(=O)O)CCN(CC(=O)O)CCN(CC(=O)O)CC2)CSCc2cccc(n2)CSC[C@@H](C(=O)NC2CCN(CC(=O)N[C@H](CCC(=O)NC[C@H](O)[C@@H](O)[C@H](O)[C@H](O)CO)C(N)=O)CC2)NC1=O]
compound DOTA-glu(AGLU)-PEG6-Asp-Trp-5Clw-Tap-Glu-leu-[Cys(2Lut)-Thp-Leu-Ile-Cys]-APAc-glu(AGLU)-NH₂ (GIP-019) of the following formula 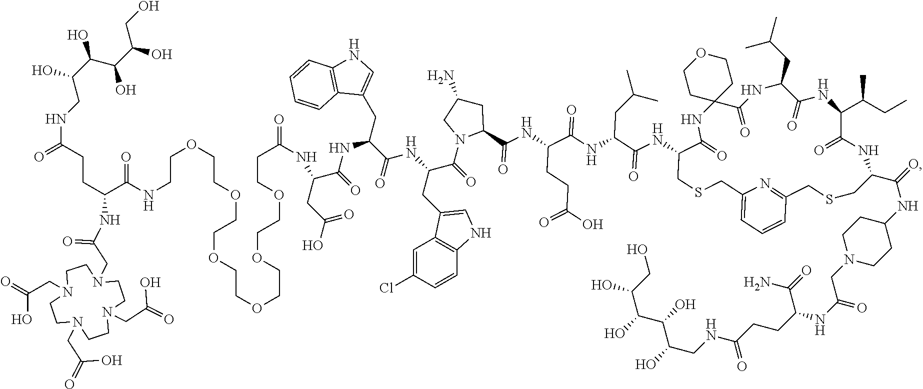 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](N)CN2C(=O)[C@H](Cc2c[nH]c3ccc(Cl)cc23)NC(=O)[C@H](Cc2c[nH]c3ccccc23)NC(=O)[C@H](CC(=O)O)NC(=O)CCOCCOCCOCCOCCOCCOCCNC(=O)[C@@H](CCC(=O)NC[C@H](O)[C@@H](O)[C@H](O)[C@H](O)CO)NC(=O)CN2CCN(CC(=O)O)CCN(CC(=O)O)CCN(CC(=O)O)CC2)CSCc2cccc(n2)CSC[C@@H](C(=O)NC2CCN(CC(=O)N[C@H](CCC(=O)NC[C@H](O)[C@@H](O)[C@H](O)[C@H](O)CO)C(N)=O)CC2)NC1=O]
compound DOTA-glu(AGLU)-O2Oc-Asp-Trp-5Clw-Tap-Glu-leu-[Cys(2Lut)-Thp-Leu-Ile-Cys]-APAc-glu(AGLU)-NH₂ (GIP-020) of the following formula 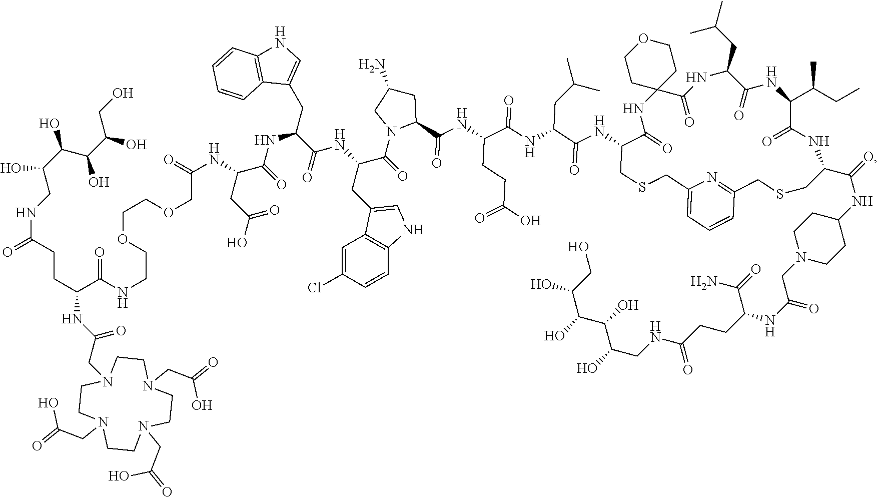 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](N)CN2C(=O)[C@H](Cc2c[nH]c3ccc(Cl)cc23)NC(=O)[C@H](Cc2c[nH]c3ccccc23)NC(=O)[C@H](CC(=O)O)NC(=O)COCCOCCNC(=O)[C@@H](CCC(=O)NC[C@H](O)[C@@H](O)[C@H](O)[C@H](O)CO)NC(=O)CN2CCN(CC(=O)O)CCN(CC(=O)O)CCN(CC(=O)O)CC2)CSCc2cccc(n2)CSC[C@@H](C(=O)NC2CCN(CC(=O)N[C@H](CCC(=O)NC[C@H](O)[C@@H](O)[C@H](O)[C@H](O)CO)C(N)=O)CC2)NC1=O]
compound HO-Succinyl-Trp-5Clw-Hyp-Glu-leu-[Cys(2Lut)-Thp-Leu-Ile-Cys]-APAc-glu(AGLU)-Lys(DOTA)-NH₂ (GIP-021) of the following formula 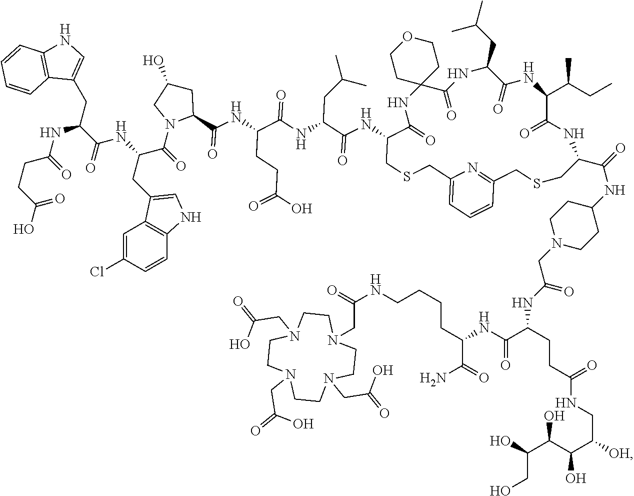 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)CCC(=O)O)CSCc2cccc(n2)CSC[C@@H](C(=O)NC2CCN(CC(=O)N[C@H](CCC(=O)NC[C@H](O)[C@@H](O)[C@H](O)[C@H](O)CO)C(=O)N[C@@H](CCCCNC(=O)CN3CCN(CC(=O)O)CCN(CC(=O)O)CCN(CC(=O)O)CC3)C(N)=O)CC2)NC1=O]
compound HO-Succinyl-Trp-5Clw-Tap-Glu-leu-[Cys(2Lut)-Apc-Leu-Ile-Cys]-APAc-glu(AGLU)-Lys(DOTA)-NH₂ (GIP-022) of the following formula 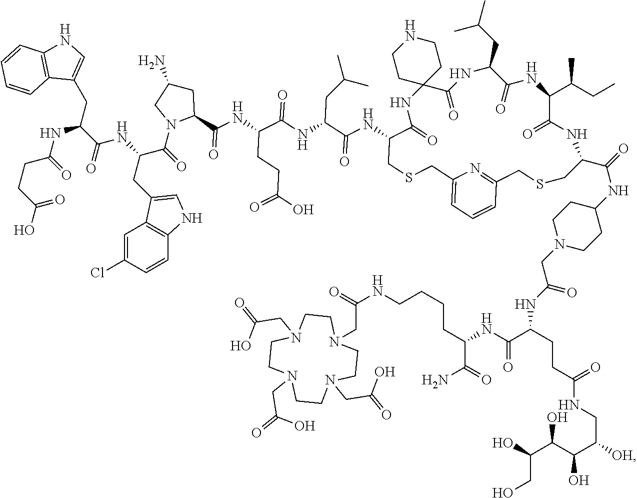 [SMILES: CC[C@H](C)[C@@H]1NC(=O)[C@H](CC(C)C)NC(=O)C2(CCNCC2)NC(=O)[C@@H](NC(=O)[C@@H](CC(C)C)NC(=O)[C@H](CCC(=O)O)NC(=O)[C@@H]2C[C@@H](N)CN2C(=O)[C@H](Cc2c[nH]c3ccc(Cl)cc23)NC(=O)[C@H](Cc2c[nH]c3ccccc23)NC(=O)CCC(=O)O)CSCc2cccc(n2)CSC[C@@H](C(=O)NC2CCN(CC(=O)N[C@H](CCC(=O)NC[C@H](O)[C@@H](O)[C@H](O)[C@H](O)CO)C(=O)N[C@@H](CCCCNC(=O)CN3CCN(CC(=O)O)CCN(CC(=O)O)CCN(CC(=O)O)CC3)C(N)=O)CC2)NC1=O]
compound HO-Succinyl-Trp-5Clw-Tap-Glu-leu-[Cys(2Lut)-Apc(DOTA)-Leu-Ile-Cys]-APAc-glu(AGLU)-NH₂ (GIP-023) of the following formula 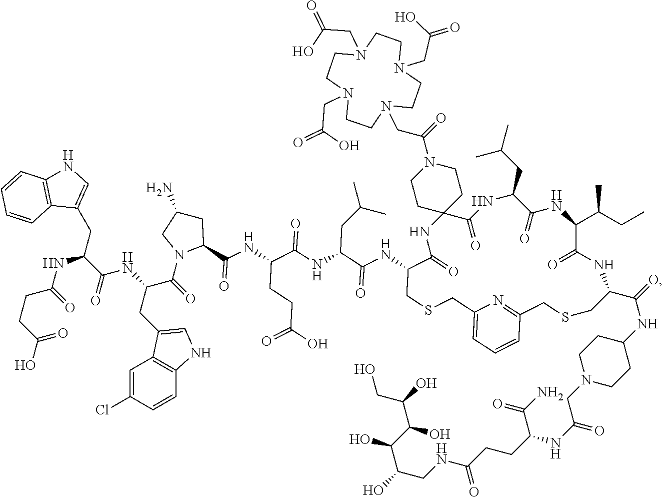 [SMILES: CC[C@H](C)[C@@H]1NC(=O)[C@H](CC(C)C)NC(=O)C2(CCN(C(=O)CN3CCN(CC(=O)O)CCN(CC(=O)O)CCN(CC(=O)O)CC3)CC2)NC(=O)[C@@H](NC(=O)[C@@H](CC(C)C)NC(=O)[C@H](CCC(=O)O)NC(=O)[C@@H]2C[C@@H](N)CN2C(=O)[C@H](Cc2c[nH]c3ccc(Cl)cc23)NC(=O)[C@H](Cc2c[nH]c3ccccc23)NC(=O)CCC(=O)O)CSCc2cccc(n2)CSC[C@@H](C(=O)NC2CCN(CC(=O)N[C@H](CCC(=O)NC[C@H](O)[C@@H](O)[C@H](O)[C@H](O)CO)C(N)=O)CC2)NC1=O]
compound AGLU-Glutar-Asp-Trp-5Clw-Tap-Glu-leu-[Cys(2Lut)-Lys(DOTA)-Leu-Ile-Cys]-APAc-glu(AGLU)-NH₂ (GIP-024) of the following formula 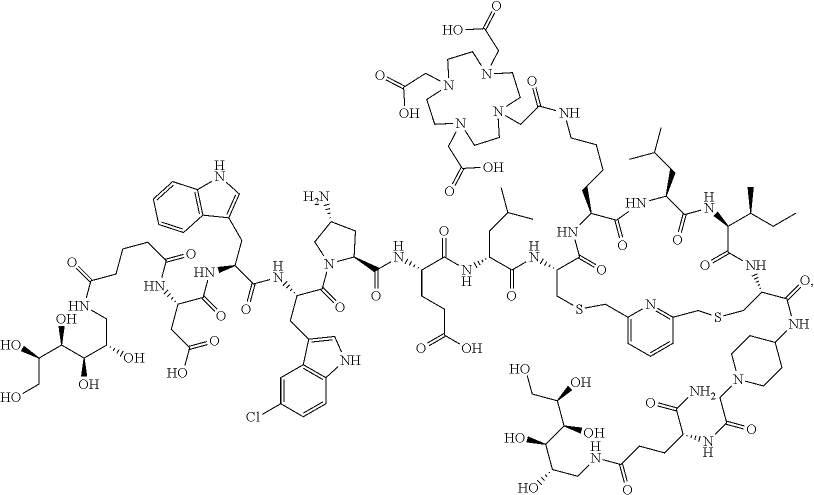 [SMILES: CC[C@H](C)[C@@H]1NC(=O)[C@H](CC(C)C)NC(=O)[C@H](CCCCNC(=O)CN2CCN(CC(=O)O)CCN(CC(=O)O)CCN(CC(=O)O)CC2)NC(=O)[C@@H](NC(=O)[C@@H](CC(C)C)NC(=O)[C@H](CCC(=O)O)NC(=O)[C@@H]2C[C@@H](N)CN2C(=O)[C@H](Cc2c[nH]c3ccc(Cl)cc23)NC(=O)[C@H](Cc2c[nH]c3ccccc23)NC(=O)[C@H](CC(=O)O)NC(=O)CCCC(=O)NC[C@H](O)[C@@H](O)[C@H](O)[C@H](O)CO)CSCc2cccc(n2)CSC[C@@H](C(=O)NC2CCN(CC(=O)N[C@H](CCC(=O)NC[C@H](O)[C@@H](O)[C@H](O)[C@H](O)CO)C(N)=O)CC2)NC1=O]
compound AGLU-Glutar-Asp-Trp-5Clw-Tap-Glu-leu-[Cys(2Lut)-lys(DOTA)-Leu-Ile-Cys]-APAc-glu(AGLU)-NH₂ (GIP-025) of the following formula 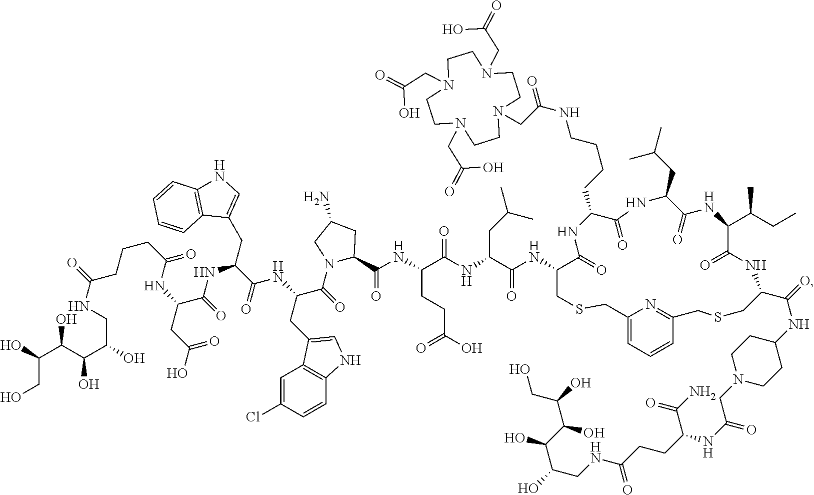 [SMILES: CC[C@H](C)[C@@H]1NC(=O)[C@H](CC(C)C)NC(=O)[C@@H](CCCCNC(=O)CN2CCN(CC(=O)O)CCN(CC(=O)O)CCN(CC(=O)O)CC2)NC(=O)[C@@H](NC(=O)[C@@H](CC(C)C)NC(=O)[C@H](CCC(=O)O)NC(=O)[C@@H]2C[C@@H](N)CN2C(=O)[C@H](Cc2c[nH]c3ccc(Cl)cc23)NC(=O)[C@H](Cc2c[nH]c3ccccc23)NC(=O)[C@H](CC(=O)O)NC(=O)CCCC(=O)NC[C@H](O)[C@@H](O)[C@H](O)[C@H](O)CO)CSCc2cccc(n2)CSC[C@@H](C(=O)NC2CCN(CC(=O)N[C@H](CCC(=O)NC[C@H](O)[C@@H](O)[C@H](O)[C@H](O)CO)C(N)=O)CC2)NC1=O]
compound AGLU-Glutar-Asp-Trp-5Clw-Tap-Glu-leu-[Cys(2Lut)-Dab(DOTA)-Leu-Ile-Cys]-APAc-glu(AGLU)-NH₂ (GIP-026) of the following formula 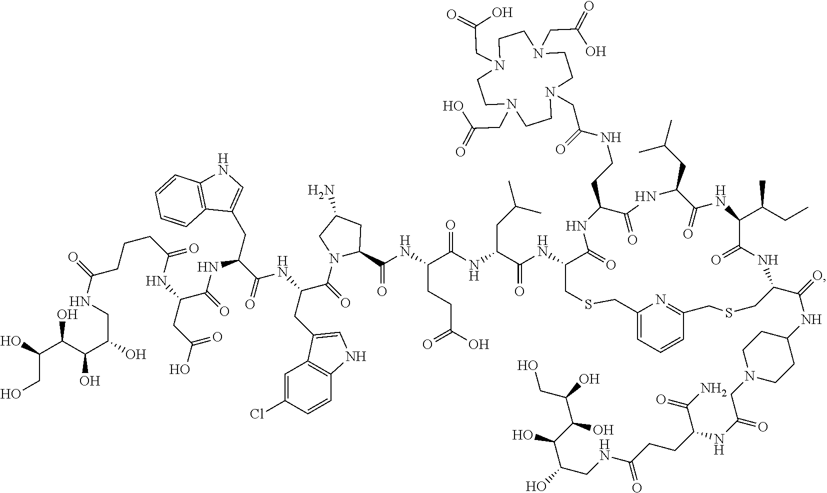 [SMILES: CC[C@H](C)[C@@H]1NC(=O)[C@H](CC(C)C)NC(=O)[C@H](CCNC(=O)CN2CCN(CC(=O)O)CCN(CC(=O)O)CCN(CC(=O)O)CC2)NC(=O)[C@@H](NC(=O)[C@@H](CC(C)C)NC(=O)[C@H](CCC(=O)O)NC(=O)[C@@H]2C[C@@H](N)CN2C(=O)[C@H](Cc2c[nH]c3ccc(Cl)cc23)NC(=O)[C@H](Cc2c[nH]c3ccccc23)NC(=O)[C@H](CC(=O)O)NC(=O)CCCC(=O)NC[C@H](O)[C@@H](O)[C@H](O)[C@H](O)CO)CSCc2cccc(n2)CSC[C@@H](C(=O)NC2CCN(CC(=O)N[C@H](CCC(=O)NC[C@H](O)[C@@H](O)[C@H](O)[C@H](O)CO)C(N)=O)CC2)NC1=O]
compound AGLU-Glutar-Asp-Trp-5Clw-Tap-Glu-leu-[Cys(2Lut)-dab(DOTA)-Leu-Ile-Cys]-APAc-glu(AGLU)-NH₂ (GIP-027) of the following formula 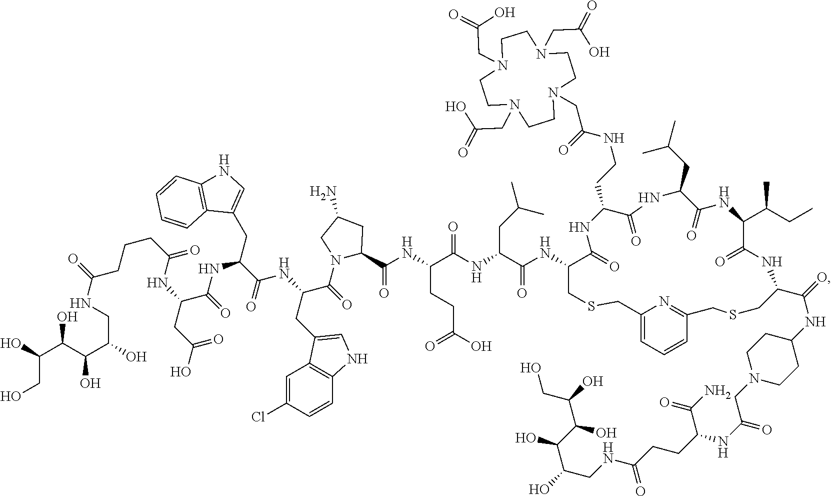 [SMILES: CC[C@H](C)[C@@H]1NC(=O)[C@H](CC(C)C)NC(=O)[C@@H](CCNC(=O)CN2CCN(CC(=O)O)CCN(CC(=O)O)CCN(CC(=O)O)CC2)NC(=O)[C@@H](NC(=O)[C@@H](CC(C)C)NC(=O)[C@H](CCC(=O)O)NC(=O)[C@@H]2C[C@@H](N)CN2C(=O)[C@H](Cc2c[nH]c3ccc(Cl)cc23)NC(=O)[C@H](Cc2c[nH]c3ccccc23)NC(=O)[C@H](CC(=O)O)NC(=O)CCCC(=O)NC[C@H](O)[C@@H](O)[C@H](O)[C@H](O)CO)CSCc2cccc(n2)CSC[C@@H](C(=O)NC2CCN(CC(=O)N[C@H](CCC(=O)NC[C@H](O)[C@@H](O)[C@H](O)[C@H](O)CO)C(N)=O)CC2)NC1=O]
compound AGLU-Glutar-Asp-Trp-5Clw-Tap-Glu-leu-[Cys(2Lut)-Apc(NODAGA)-Leu-Ile-Cys]-APAc-glu(AGLU)-NH₂ (GIP-028) of the following formula 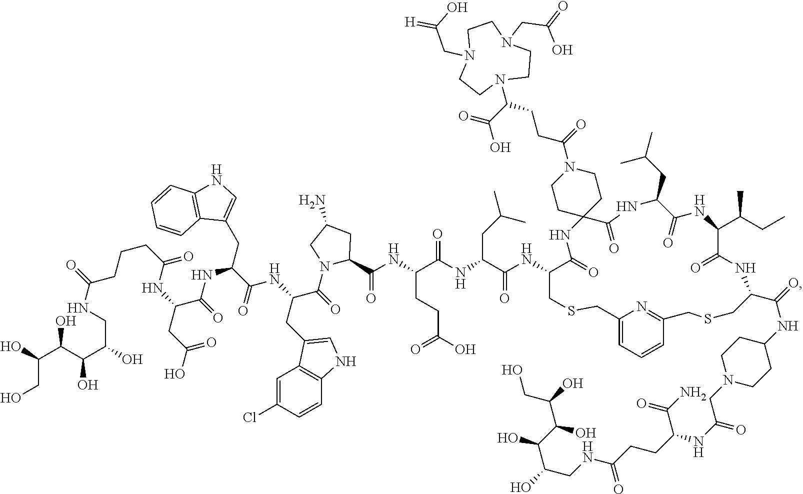
compound AGLU-Glutar-Asp-Trp-5Clw-Tap-Glu-leu-[Cys(2Lut)-Apc(NOPO)-Leu-Ile-Cys]-APAc-glu(AGLU)-NH₂ (GIP-029) of the following formula 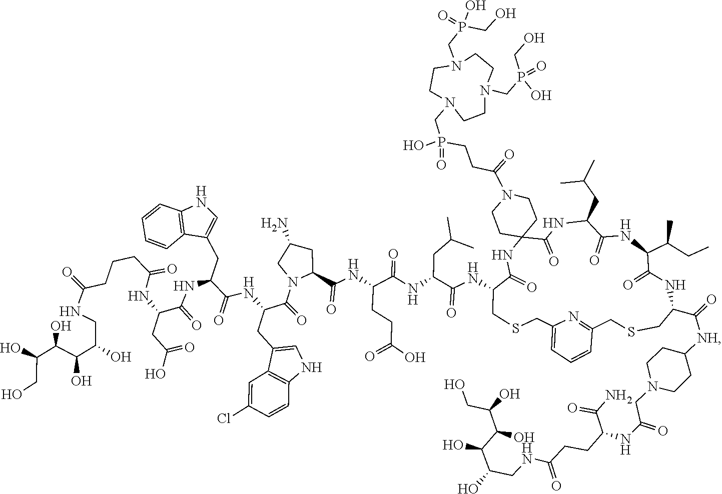 [SMILES: CC[C@H](C)[C@@H]1NC(=O)[C@H](CC(C)C)NC(=O)C2(CCN(C(=O)CCP(=O)(O)CN3CCN(CP(=O)(O)CO)CCN(CP(=O)(O)CO)CC3)CC2)NC(=O)[C@@H](NC(=O)[C@@H](CC(C)C)NC(=O)[C@H](CCC(=O)O)NC(=O)[C@@H]2C[C@@H](N)CN2C(=O)[C@H](Cc2c[nH]c3ccc(Cl)cc23)NC(=O)[C@H](Cc2c[nH]c3ccccc23)NC(=O)[C@H](CC(=O)O)NC(=O)CCCC(=O)NC[C@H](O)[C@@H](O)[C@H](O)[C@H](O)CO)CSCc2cccc(n2)CSC[C@@H](C(=O)NC2CCN(CC(=O)N[C@H](CCC(=O)NC[C@H](O)[C@@H](O)[C@H](O)[C@H](O)CO)C(N)=O)CC2)NC1=O]
compound AGLU-Glutar-Asp-Trp-5Clw-Tap-Glu-leu-[Cys(2Lut)-Apc(N₄Ac)-Leu-Ile-Cys]-APAc-glu(AGLU)-NH₂ (GIP-030) of the following formula 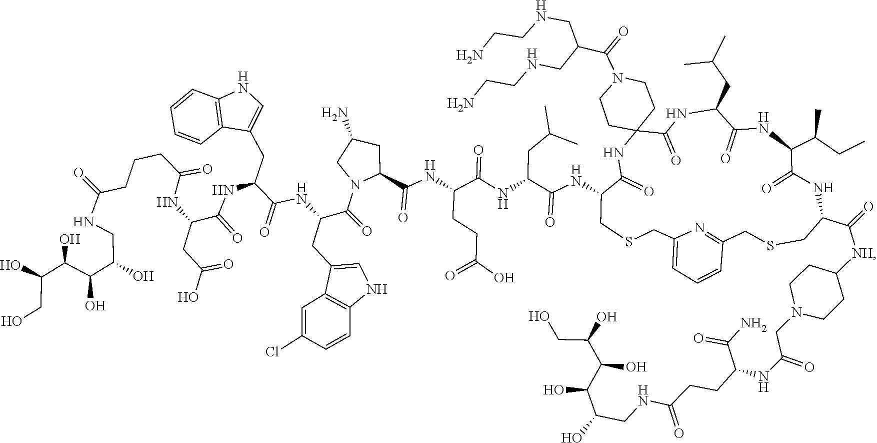 [SMILES: CC[C@H](C)[C@@H]1NC(=O)[C@H](CC(C)C)NC(=O)C2(CCN(C(=O)C(CNCCN)CNCCN)CC2)NC(=O)[C@@H](NC(=O)[C@@H](CC(C)C)NC(=O)[C@H](CCC(=O)O)NC(=O)[C@@H]2C[C@@H](N)CN2C(=O)[C@H](Cc2c[nH]c3ccc(Cl)cc23)NC(=O)[C@H](Cc2c[nH]c3ccccc23)NC(=O)[C@H](CC(=O)O)NC(=O)CCCC(=O)NC[C@H](O)[C@@H](O)[C@H](O)[C@H](O)CO)CSCc2cccc(n2)CSC[C@@H](C(=O)NC2CCN(CC(=O)N[C@H](CCC(=O)NC[C@H](O)[C@@H](O)[C@H](O)[C@H](O)CO)C(N)=O)CC2)NC1=O]
compound AGLU-Glutar-Asp-Trp-5Clw-Tap-Glu-leu-[Cys(2Lut)-Apc(NODAGA-Gly)-Leu-Ile-Cys]-APAc-glu(AGLU)-NH₂ (GIP-031) of the following formula 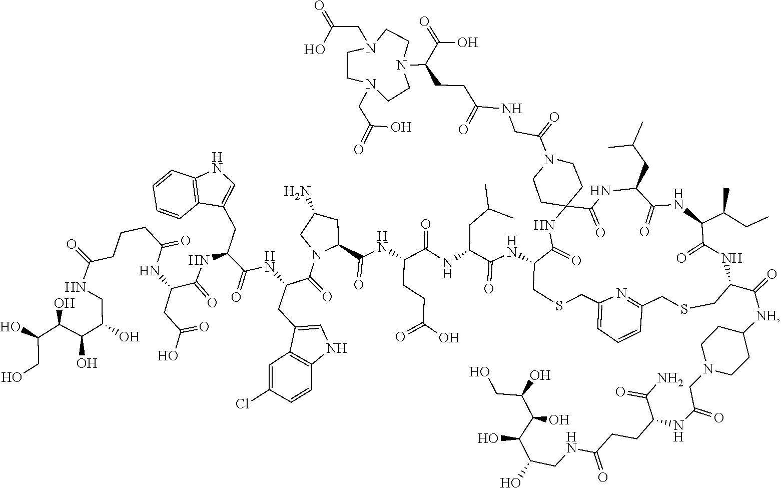 [SMILES: CC[C@H](C)[C@@H]1NC(=O)[C@H](CC(C)C)NC(=O)C2(CCN(C(=O)CNC(=O)CC[C@H](C(=O)O)N3CCN(CC(=O)O)CCN(CC(=O)O)CC3)CC2)NC(=O)[C@@H](NC(=O)[C@@H](CC(C)C)NC(=O)[C@H](CCC(=O)O)NC(=O)[C@@H]2C[C@@H](N)CN2C(=O)[C@H](Cc2c[nH]c3ccc(Cl)cc23)NC(=O)[C@H](Cc2c[nH]c3ccccc23)NC(=O)[C@H](CC(=O)O)NC(=O)CCCC(=O)NC[C@H](O)[C@@H](O)[C@H](O)[C@H](O)CO)CSCc2cccc(n2)CSC[C@@H](C(=O)NC2CCN(CC(=O)N[C@H](CCC(=O)NC[C@H](O)[C@@H](O)[C@H](O)[C@H](O)CO)C(N)=O)CC2)NC1=O]
compound AGLU-Glutar-APAc-Asp-Trp-5Clw-Hyp-Glu-leu-[Cys(2Lut)-Apc(DOTA)-Leu-Ile-Cys]-glu(AGLU)-NH₂ (GIP-033) of the following formula 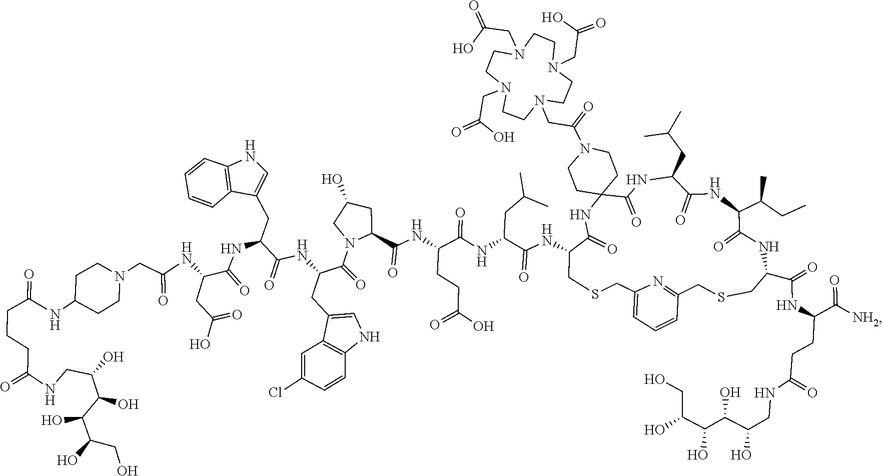 [SMILES: CC[C@H](C)[C@@H]1NC(=O)[C@H](CC(C)C)NC(=O)C2(CCN(C(=O)CN3CCN(CC(=O)O)CCN(CC(=O)O)CCN(CC(=O)O)CC3)CC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CC(=O)O)NC(=O)CN2CCC(NC(=O)CCCC(=O)NC[C@H](O)[C@@H](O)[C@H](O)[C@H](O)CO)CC2)CSCc2cccc(n2)CSC[C@@H](C(=O)N[C@H](CCC(=O)NC[C@H](O)[C@@H](O)[C@H](O)[C@H](O)CO)C(N)=O)NC1=O]
compound AGLU-Glutar-Hse-Trp-5Clw-Tap-Glu-leu-[Cys(2Lut)-Apc(DOTA)-Leu-Ile-Cys]-APAc-glu(AGLU)-NH₂ (GIP-034) of the following formula 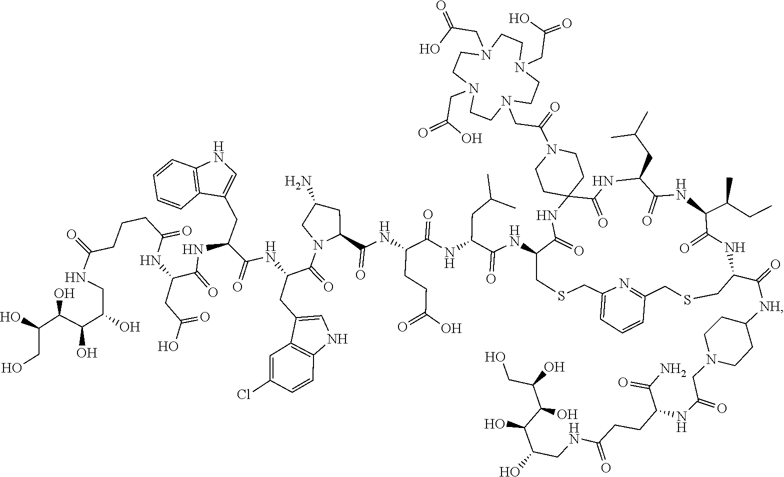 [SMILES: CC[C@H](C)[C@@H]1NC(=O)[C@H](CC(C)C)NC(=O)C2(CCN(C(=O)CN3CCN(CC(=O)O)CCN(CC(=O)O)CCN(CC(=O)O)CC3)CC2)NC(=O)[C@H](NC(=O)[C@@H](CC(C)C)NC(=O)[C@H](CCC(=O)O)NC(=O)[C@@H]2C[C@@H](N)CN2C(=O)[C@H](Cc2c[nH]c3ccc(Cl)cc23)NC(=O)[C@H](Cc2c[nH]c3ccccc23)NC(=O)[C@H](CC(=O)O)NC(=O)CCCC(=O)NC[C@H](O)[C@@H](O)[C@H](O)[C@H](O)CO)CSCc2cccc(n2)CSC[C@@H](C(=O)NC2CCN(CC(=O)N[C@H](CCC(=O)NC[C@H](O)[C@@H](O)[C@H](O)[C@H](O)CO)C(N)=O)CC2)NC1=O]
compound AGLU-Glutar-Asp-Trp-5Clw-Tap-Glu-Leu-[Cys(2Lut)-Apc(DOTA)-Leu-Ile-Cys]-APAc-glu(AGLU)-NH₂ (GIP-035) of the following formula 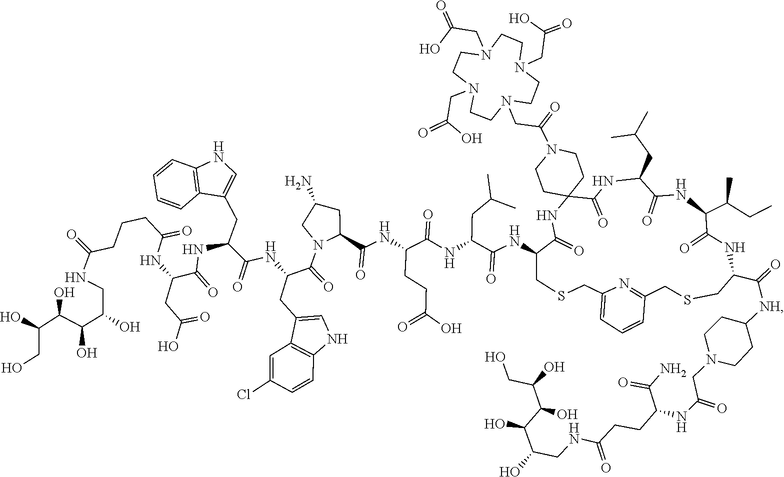 [SMILES: CC[C@H](C)[C@@H]1NC(=O)[C@H](CC(C)C)NC(=O)C2(CCN(C(=O)CN3CCN(CC(=O)O)CCN(CC(=O)O)CCN(CC(=O)O)CC3)CC2)NC(=O)[C@H](NC(=O)[C@@H](CC(C)C)NC(=O)[C@H](CCC(=O)O)NC(=O)[C@@H]2C[C@@H](N)CN2C(=O)[C@H](Cc2c[nH]c3ccc(Cl)cc23)NC(=O)[C@H](Cc2c[nH]c3ccccc23)NC(=O)[C@H](CC(=O)O)NC(=O)CCCC(=O)NC[C@H](O)[C@@H](O)[C@H](O)[C@H](O)CO)CSCc2cccc(n2)CSC[C@@H](C(=O)NC2CCN(CC(=O)N[C@H](CCC(=O)NC[C@H](O)[C@@H](O)[C@H](O)[C@H](O)CO)C(N)=O)CC2)NC1=O]
compound DOTA-glu(AGLU)-Asp-Trp-5Clw-Hyp-Glu-leu-[Cys(2Lut)-Thp-Leu-Ile-Cys]-APAc-glu(AGLU)-NH₂ (GIP-036) of the following formula 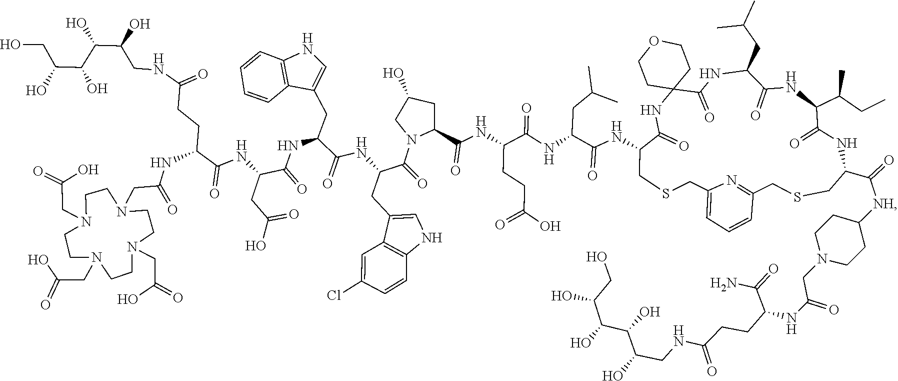 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CC(=O)O)NC(=O)[C@@H](CCC(=O)NC[C@H](O)[C@@H](O)[C@H](O)[C@H](O)CO)NC(=O)CN2CCN(CC(=O)O)CCN(CC(=O)O)CCN(CC(=O)O)CC2)CSCc2cccc(n2)CSC[C@@H](C(=O)NC2CCN(CC(=O)N[C@H](CCC(=O)NC[C@H](O)[C@@H](O)[C@H](O)[C@H](O)CO)C(N)=O)CC2)NC1=O]
compound DOTA-PEG6-glu(AGLU)-Asp-Trp-5Clw-Tap-Glu-leu-[Cys(2Lut)-Thp-Leu-Ile-Cys]-APAc-glu(AGLU)-NH₂ (GIP-037) of the following formula 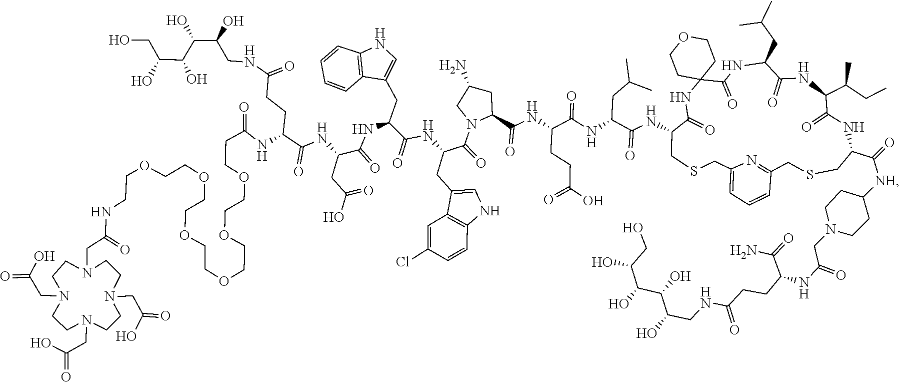 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](N)CN2C(=O)[C@H](Cc2c[nH]c3ccc(Cl)cc23)NC(=O)[C@H](Cc2c[nH]c3ccccc23)NC(=O)[C@H](CC(=O)O)NC(=O)[C@@H](CCC(=O)NC[C@H](O)[C@@H](O)[C@H](O)[C@H](O)CO)NC(=O)CCOCCOCCOCCOCCOCCOCCNC(=O)CN2CCN(CC(=O)O)CCN(CC(=O)O)CCN(CC(=O)O)CC2)CSCc2cccc(n2)CSC[C@@H](C(=O)NC2CCN(CC(=O)N[C@H](CCC(=O)NC[C@H](O)[C@@H](O)[C@H](O)[C@H](O)CO)C(N)=O)CC2)NC1=O]
compound AGLU-Glutar-Asp-Trp-5Clw-Tap-Glu-leu-[Cys(2Lut)-Apc(DOTA)-Leu-Ile-cys]-APAc-glu(AGLU)-NH₂ (GIP-038) of the following formula 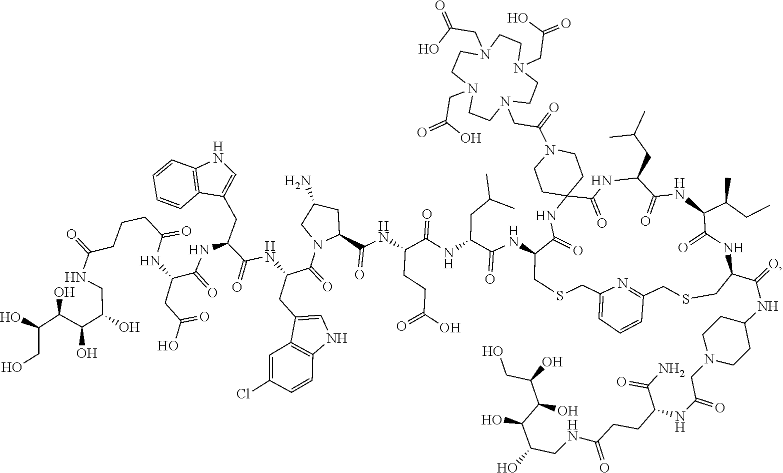 [SMILES: CC[C@H](C)[C@@H]1NC(=O)[C@H](CC(C)C)NC(=O)C2(CCN(C(=O)CN3CCN(CC(=O)O)CCN(CC(=O)O)CCN(CC(=O)O)CC3)CC2)NC(=O)[C@H](NC(=O)[C@@H](CC(C)C)NC(=O)[C@H](CCC(=O)O)NC(=O)[C@@H]2C[C@@H](N)CN2C(=O)[C@H](Cc2c[nH]c3ccc(Cl)cc23)NC(=O)[C@H](Cc2c[nH]c3ccccc23)NC(=O)[C@H](CC(=O)O)NC(=O)CCCC(=O)NC[C@H](O)[C@@H](O)[C@H](O)[C@H](O)CO)CSCc2cccc(n2)CSC[C@H](C(=O)NC2CCN(CC(=O)N[C@H](CCC(=O)NC[C@H](O)[C@@H](O)[C@H](O)[C@H](O)CO)C(N)=O)CC2)NC1=O]
compound AGLU-Glutar-APAc-Asp-Trp-5Clw-Hyp-Glu-leu-[Cys(2Lut)-Apc(DOTA)-Leu-Ile-Cys]-lys(MeO36PEG)-NH₂ (GIP-039) of the following formula 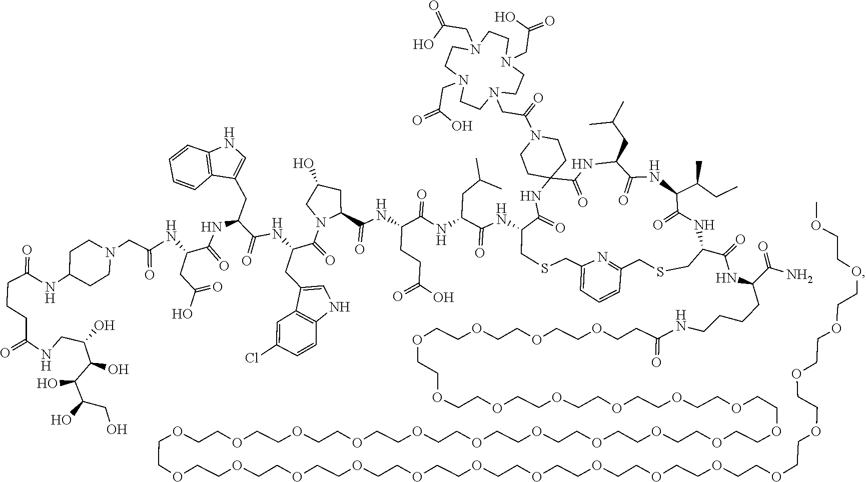
compound MeO36PEG-APAc-Asp-Trp-5Clw-Hyp-Glu-leu-[Cys(2Lut)-Apc(DOTA)-Leu-Ile-Cys]-glu(AGLU)-NH₂ (GIP-040) of the following formula 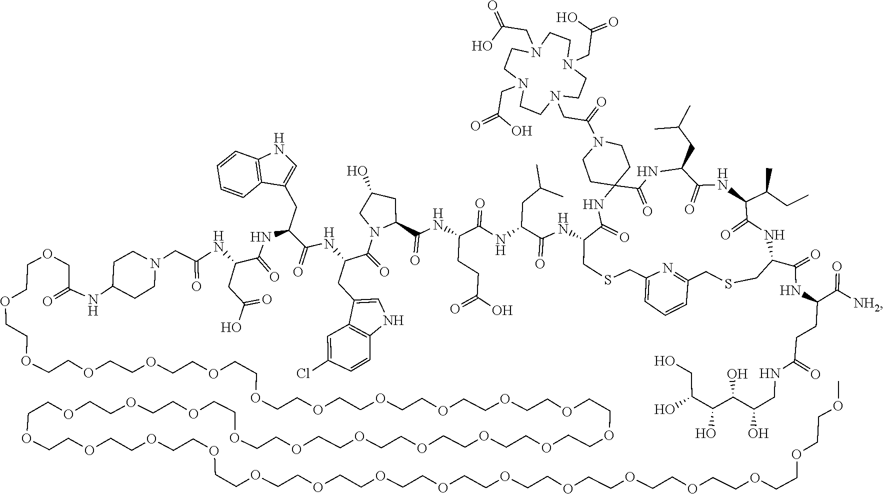 [SMILES: CC[C@H](C)[C@@H]1NC(=O)[C@H](CC(C)C)NC(=O)C2(CCN(C(=O)CN3CCN(CC(=O)O)CCN(CC(=O)O)CCN(CC(=O)O)CC3)CC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CC(=O)O)NC(=O)CN2CCC(NC(=O)COCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOC)CC2)CSCc2cccc(n2)CSC[C@@H](C(=O)N[C@H](CCC(=O)NC[C@H](O)[C@@H](O)[C@H](O)[C@H](O)CO)C(N)=O)NC1=O]
compound MeO36PEG-APAc-Asp-Trp-5Clw-Hyp-Glu-leu-[Cys(2Lut)-Apc(DOTA)-Leu-Ile-Cys]-APAc-glu(AGLU)-NH₂ (GIP-041) of the following formula 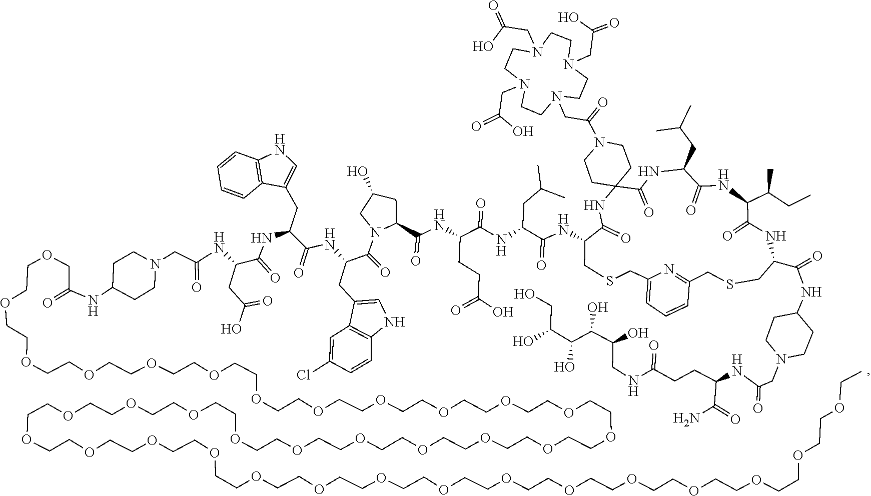
compound MeO36PEG-APAc-Asp-Trp-5Clw-Tap-Glu-leu-[Cys(2Lut)-Apc(DOTA)-Leu-Ile-Cys]-APAc-glu(AGLU)-NH₂ (GIP-042) of the following formula 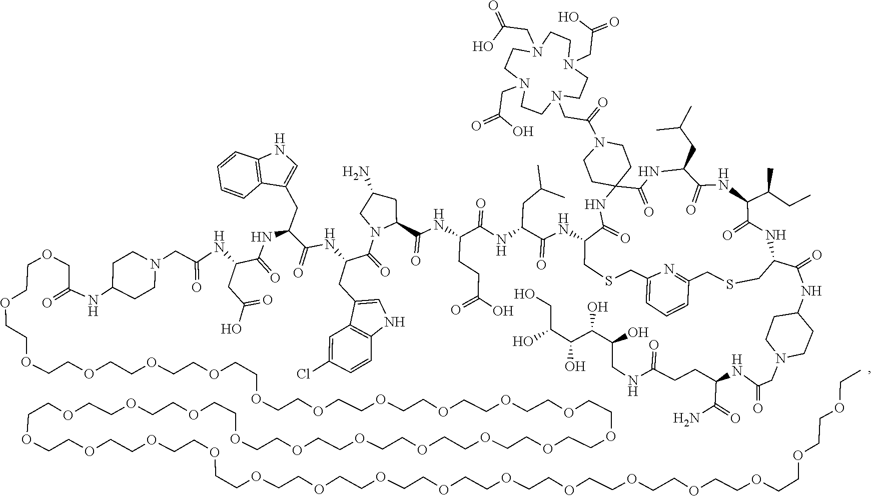
compound AGLU-Glutar-APAc-Asp-Trp-5Clw-Hyp-Glu-leu-[Cys(2Lut)-Apc(DOTA-glu(AGLU))-Leu-Ile-Cys]-APAc-glu(AGLU)-NH₂ (GIP-043) of the following formula 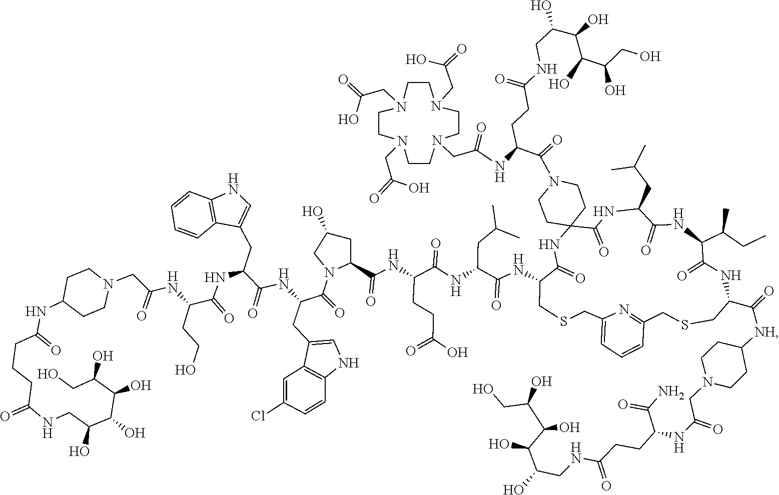 [SMILES: CC[C@H](C)[C@@H]1NC(=O)[C@H](CC(C)C)NC(=O)C2(CCN(C(=O)[C@H](CCC(=O)NC[C@H](O)[C@@H](O)[C@H](O)[C@H](O)CO)NC(=O)CN3CCN(CC(=O)O)CCN(CC(=O)O)CCN(CC(=O)O)CC3)CC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CCO)NC(=O)CN2CCC(NC(=O)CCCC(=O)NC[C@H](O)[C@@H](O)[C@H](O)[C@H](O)CO)CC2)CSCc2cccc(n2)CSC[C@@H](C(=O)NC2CCN(CC(=O)N[C@H](CCC(=O)NC[C@H](O)[C@@H](O)[C@H](O)[C@H](O)CO)C(N)=O)CC2)NC1=O]
compound DOTA-O2Oc-glu(AGLU)-Asp-Trp-5Clw-Tap-Glu-leu-[Cys(2Lut)-Apc(AGLU-Glutar)-Leu-Ile-Cys]-APAc-glu(AGLU)-NH₂ (GIP-044) of the following formula 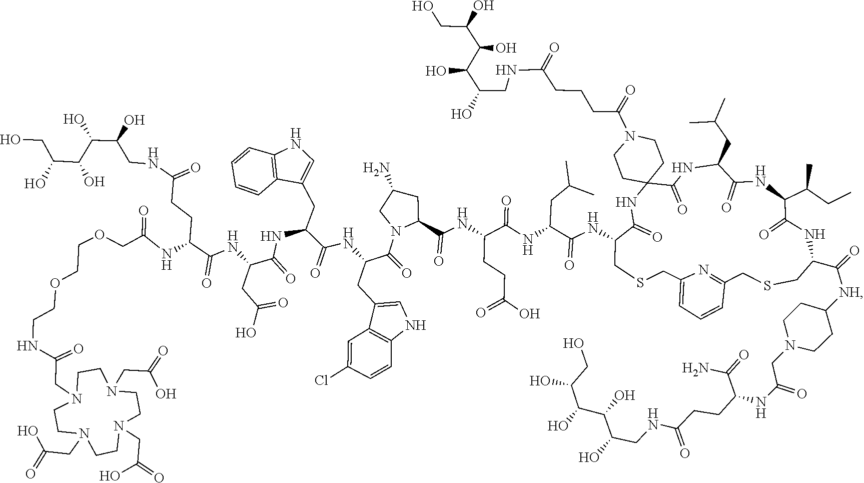 [SMILES: CC[C@H](C)[C@@H]1NC(=O)[C@H](CC(C)C)NC(=O)C2(CCN(C(=O)CCCC(=O)NC[C@H](O)[C@@H](O)[C@H](O)[C@H](O)CO)CC2)NC(=O)[C@@H](NC(=O)[C@@H](CC(C)C)NC(=O)[C@H](CCC(=O)O)NC(=O)[C@@H]2C[C@@H](N)CN2C(=O)[C@H](Cc2c[nH]c3ccc(Cl)cc23)NC(=O)[C@H](Cc2c[nH]c3ccccc23)NC(=O)[C@H](CC(=O)O)NC(=O)[C@@H](CCC(=O)NC[C@H](O)[C@@H](O)[C@H](O)[C@H](O)CO)NC(=O)COCCOCCNC(=O)CN2CCN(CC(=O)O)CCN(CC(=O)O)CCN(CC(=O)O)CC2)CSCc2cccc(n2)CSC[C@@H](C(=O)NC2CCN(CC(=O)N[C@H](CCC(=O)NC[C@H](O)[C@@H](O)[C@H](O)[C@H](O)CO)C(N)=O)CC2)NC1=O]
compound DOTA-glu(AGLU)-Asp-Trp-5Clw-Tap-Glu-leu-[Cys(2Lut)-Apc(AGLU-Glutar)-Leu-Ile-Cys]-APAc-glu(AGLU)-NH₂ (GIP-045) of the following formula 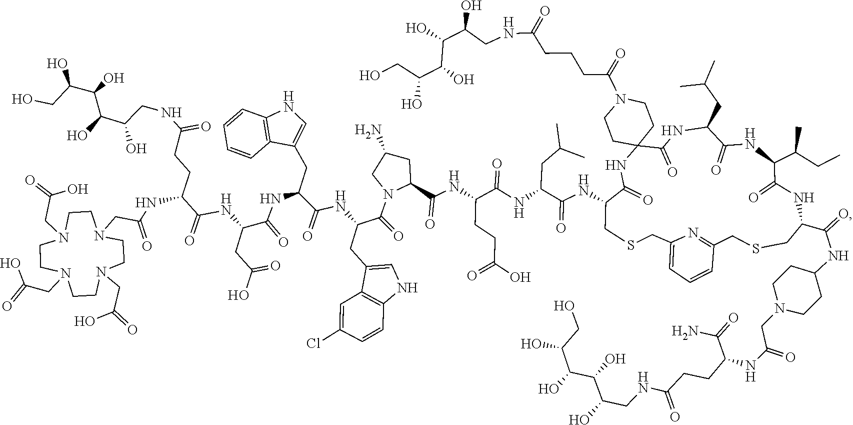 [SMILES: CC[C@H](C)[C@@H]1NC(=O)[C@H](CC(C)C)NC(=O)C2(CCN(C(=O)CCCC(=O)NC[C@H](O)[C@@H](O)[C@H](O)[C@H](O)CO)CC2)NC(=O)[C@@H](NC(=O)[C@@H](CC(C)C)NC(=O)[C@H](CCC(=O)O)NC(=O)[C@@H]2C[C@@H](N)CN2C(=O)[C@H](Cc2c[nH]c3ccc(Cl)cc23)NC(=O)[C@H](Cc2c[nH]c3ccccc23)NC(=O)[C@H](CC(=O)O)NC(=O)[C@@H](CCC(=O)NC[C@H](O)[C@@H](O)[C@H](O)[C@H](O)CO)NC(=O)CN2CCN(CC(=O)O)CCN(CC(=O)O)CCN(CC(=O)O)CC2)CSCc2cccc(n2)CSC[C@@H](C(=O)NC2CCN(CC(=O)N[C@H](CCC(=O)NC[C@H](O)[C@@H](O)[C@H](O)[C@H](O)CO)C(N)=O)CC2)NC1=O]
compound AGLU-Glutar-APAc-Ser-Trp-5Clw-Tap(AGLU-Glutar)-Hse-leu-[Cys(2Lut)-Apc(DOTA)-Leu-Ile-Cys]-APAc-glu(AGLU)-NH₂ (GIP-046) of the following formula 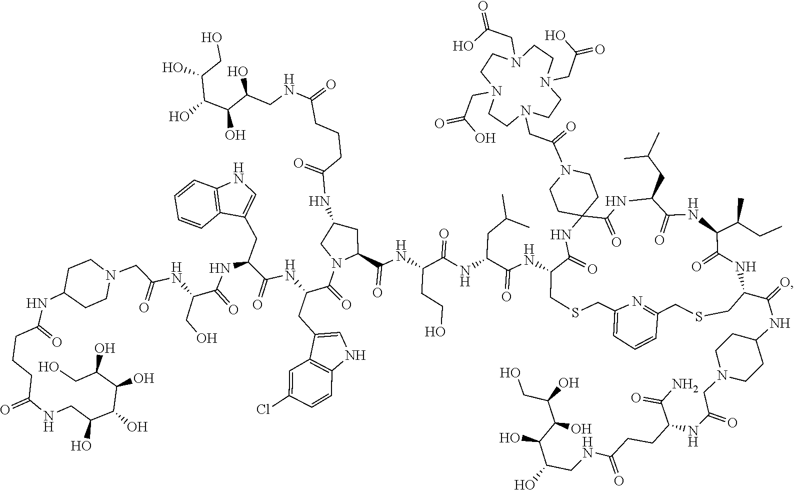 [SMILES: CC[C@H](C)[C@@H]1NC(=O)[C@H](CC(C)C)NC(=O)C2(CCN(C(=O)CN3CCN(CC(=O)O)CCN(CC(=O)O)CCN(CC(=O)O)CC3)CC2)NC(=O)[C@@H](NC(=O)[C@@H](CC(C)C)NC(=O)[C@H](CCO)NC(=O)[C@@H]2C[C@@H](NC(=O)CCCC(=O)NC[C@H](O)[C@@H](O)[C@H](O)[C@H](O)CO)CN2C(=O)[C@H](Cc2c[nH]c3ccc(Cl)cc23)NC(=O)[C@H](Cc2c[nH]c3ccccc23)NC(=O)[C@H](CO)NC(=O)CN2CCC(NC(=O)CCCC(=O)NC[C@H](O)[C@@H](O)[C@H](O)[C@H](O)CO)CC2)CSCc2cccc(n2)CSC[C@@H](C(=O)NC2CCN(CC(=O)N[C@H](CCC(=O)NC[C@H](O)[C@@H](O)[C@H](O)[C@H](O)CO)C(N)=O)CC2)NC1=O]
compound AGLU-Glutar-APAc-Hse-Trp-5Clw-Hyp-Glu-leu-[Cys(2Lut)-Apc(DOTA-glu(AGLU))-Leu-Ile-Cys]-APAc-glu(AGLU)-NH₂ (GIP-047) of the following formula 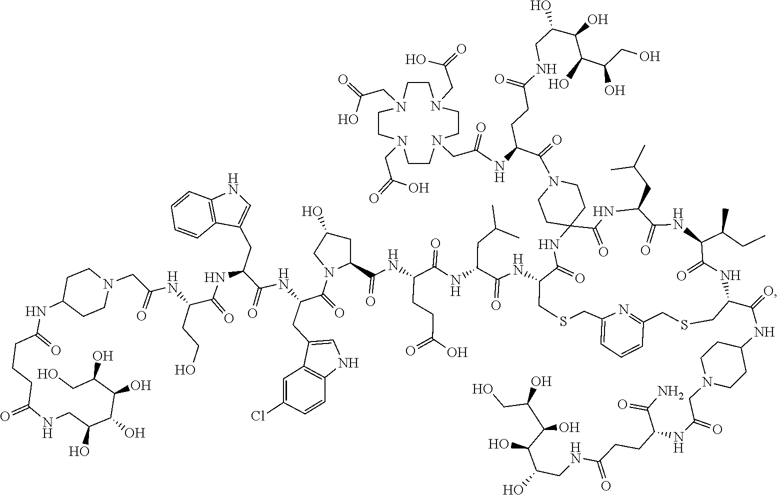 [SMILES: CC[C@H](C)[C@@H]1NC(=O)[C@H](CC(C)C)NC(=O)C2(CCN(C(=O)[C@H](CCC(=O)NC[C@H](O)[C@@H](O)[C@H](O)[C@H](O)CO)NC(=O)CN3CCN(CC(=O)O)CCN(CC(=O)O)CCN(CC(=O)O)CC3)CC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CCO)NC(=O)CN2CCC(NC(=O)CCCC(=O)NC[C@H](O)[C@@H](O)[C@H](O)[C@H](O)CO)CC2)CSCc2cccc(n2)CSC[C@@H](C(=O)NC2CCN(CC(=O)N[C@H](CCC(=O)NC[C@H](O)[C@@H](O)[C@H](O)[C@H](O)CO)C(N)=O)CC2)NC1=O]
compound AGLU-Glutar-APAc-Ser-Trp-5Clw-Tap-Hse-leu-[Cys(2Lut)-Apc(DOTA)-Leu-Ile-Cys]-APAc-glu(AGLU)-NH₂ (GIP-048) of the following formula 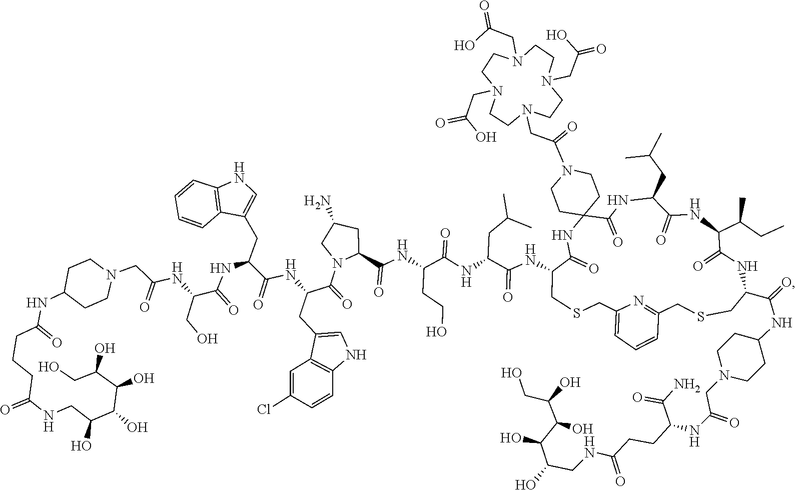 [SMILES: CC[C@H](C)[C@@H]1NC(=O)[C@H](CC(C)C)NC(=O)C2(CCN(C(=O)CN3CCN(CC(=O)O)CCN(CC(=O)O)CCN(CC(=O)O)CC3)CC2)NC(=O)[C@@H](NC(=O)[C@@H](CC(C)C)NC(=O)[C@H](CCO)NC(=O)[C@@H]2C[C@@H](N)CN2C(=O)[C@H](Cc2c[nH]c3ccc(Cl)cc23)NC(=O)[C@H](Cc2c[nH]c3ccccc23)NC(=O)[C@H](CO)NC(=O)CN2CCC(NC(=O)CCCC(=O)NC[C@H](O)[C@@H](O)[C@H](O)[C@H](O)CO)CC2)CSCc2cccc(n2)CSC[C@@H](C(=O)NC2CCN(CC(=O)N[C@H](CCC(=O)NC[C@H](O)[C@@H](O)[C@H](O)[C@H](O)CO)C(N)=O)CC2)NC1=O]
compound AGLU-Glutar-APAc-Hse-Trp-5Clw-Hyp-Glu-leu-[Cys(2Lut)-Apc(DOTA)-Leu-Ile-Cys]-APAc-glu(AGLU)-NH₂ (GIP-049) of the following formula 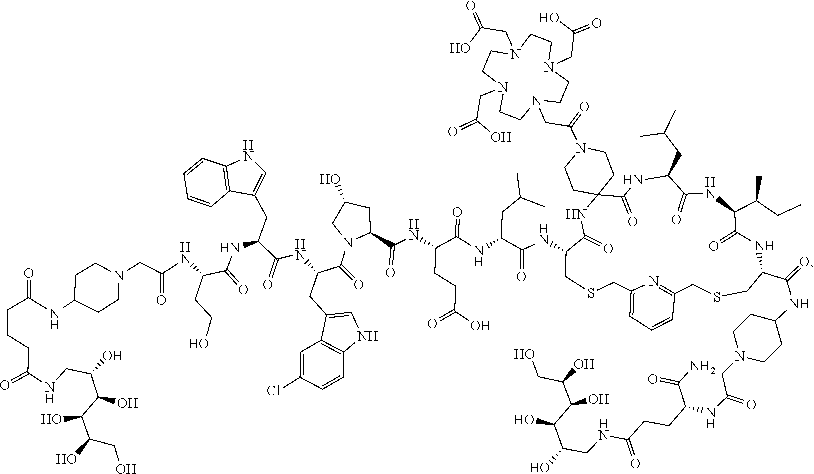 [SMILES: CC[C@H](C)[C@@H]1NC(=O)[C@H](CC(C)C)NC(=O)C2(CCN(C(=O)CN3CCN(CC(=O)O)CCN(CC(=O)O)CCN(CC(=O)O)CC3)CC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CCO)NC(=O)CN2CCC(NC(=O)CCCC(=O)NC[C@H](O)[C@@H](O)[C@H](O)[C@H](O)CO)CC2)CSCc2cccc(n2)CSC[C@@H](C(=O)NC2CCN(CC(=O)N[C@H](CCC(=O)NC[C@H](O)[C@@H](O)[C@H](O)[C@H](O)CO)C(N)=O)CC2)NC1=O]
compound AGLU-Glutar-APAc-Hse-Trp-5Clw-Tap-Glu-leu-[Cys(2Lut)-Apc(DOTA)-Leu-Ile-Cys]-APAc-glu(AGLU)-NH₂ (GIP-050) of the following formula 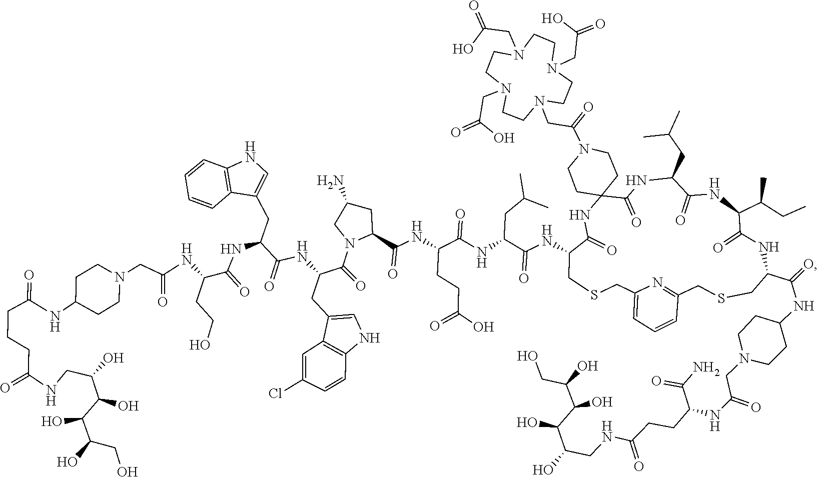 [SMILES: CC[C@H](C)[C@@H]1NC(=O)[C@H](CC(C)C)NC(=O)C2(CCN(C(=O)CN3CCN(CC(=O)O)CCN(CC(=O)O)CCN(CC(=O)O)CC3)CC2)NC(=O)[C@@H](NC(=O)[C@@H](CC(C)C)NC(=O)[C@H](CCC(=O)O)NC(=O)[C@@H]2C[C@@H](N)CN2C(=O)[C@H](Cc2c[nH]c3ccc(Cl)cc23)NC(=O)[C@H](Cc2c[nH]c3ccccc23)NC(=O)[C@H](CCO)NC(=O)CN2CCC(NC(=O)CCCC(=O)NC[C@H](O)[C@@H](O)[C@H](O)[C@H](O)CO)CC2)CSCc2cccc(n2)CSC[C@@H](C(=O)NC2CCN(CC(=O)N[C@H](CCC(=O)NC[C@H](O)[C@@H](O)[C@H](O)[C@H](O)CO)C(N)=O)CC2)NC1=O]
compound AGLU-Glutar-Asp-Trp-5Clw-Tap-Glu-leu-[cys(2Lut)-Apc(DOTA)-Leu-Ile-Cys]-APAc-glu(AGLU)-NH₂ (GIP-051) of the following formula 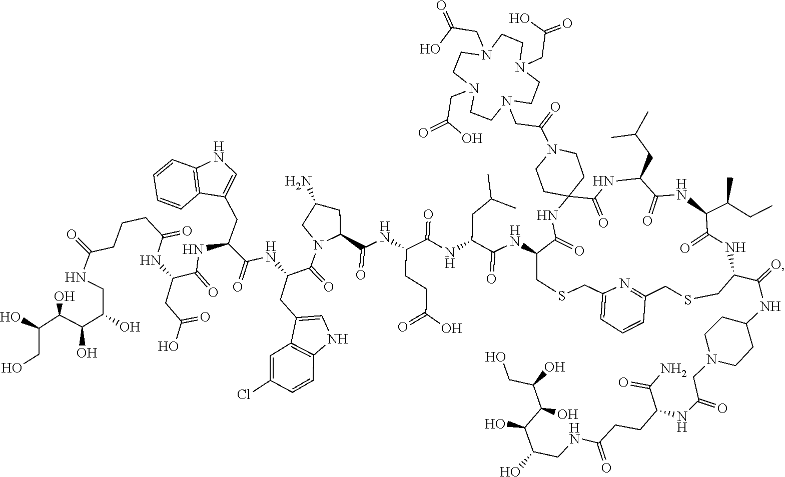 [SMILES: CC[C@H](C)[C@@H]1NC(=O)[C@H](CC(C)C)NC(=O)C2(CCN(C(=O)CN3CCN(CC(=O)O)CCN(CC(=O)O)CCN(CC(=O)O)CC3)CC2)NC(=O)[C@H](NC(=O)[C@@H](CC(C)C)NC(=O)[C@H](CCC(=O)O)NC(=O)[C@@H]2C[C@@H](N)CN2C(=O)[C@H](Cc2c[nH]c3ccc(Cl)cc23)NC(=O)[C@H](Cc2c[nH]c3ccccc23)NC(=O)[C@H](CC(=O)O)NC(=O)CCCC(=O)NC[C@H](O)[C@@H](O)[C@H](O)[C@H](O)CO)CSCc2cccc(n2)CSC[C@@H](C(=O)NC2CCN(CC(=O)N[C@H](CCC(=O)NC[C@H](O)[C@@H](O)[C@H](O)[C@H](O)CO)C(N)=O)CC2)NC1=O]
compound AGLU-Glutar-Asp-Trp-5Clw-Tap-Glu-leu-[cys(2Lut)-Apc(DOTA)-Leu-Ile-cys]-APAc-glu(AGLU)-NH₂ (GIP-052) of the following formula 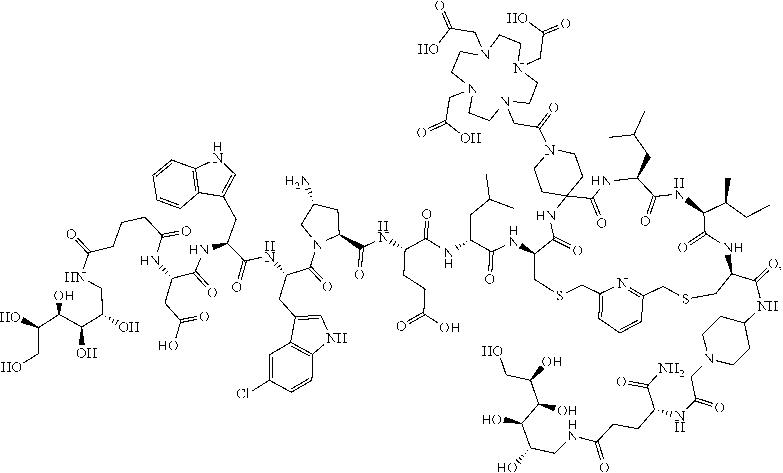 [SMILES: CC[C@H](C)[C@@H]1NC(=O)[C@H](CC(C)C)NC(=O)C2(CCN(C(=O)CN3CCN(CC(=O)O)CCN(CC(=O)O)CCN(CC(=O)O)CC3)CC2)NC(=O)[C@H](NC(=O)[C@@H](CC(C)C)NC(=O)[C@H](CCC(=O)O)NC(=O)[C@@H]2C[C@@H](N)CN2C(=O)[C@H](Cc2c[nH]c3ccc(Cl)cc23)NC(=O)[C@H](Cc2c[nH]c3ccccc23)NC(=O)[C@H](CC(=O)O)NC(=O)CCCC(=O)NC[C@H](O)[C@@H](O)[C@H](O)[C@H](O)CO)CSCc2cccc(n2)CSC[C@H](C(=O)NC2CCN(CC(=O)N[C@H](CCC(=O)NC[C@H](O)[C@@H](O)[C@H](O)[C@H](O)CO)C(N)=O)CC2)NC1=O]
compound DOTA-Ttds-Asp-Trp-5Clw-Pro-Glu-leu-[Cys(3MeBn)-Apc(DOTA)-Leu-Ile-AET] (GIP-053) of the following formula 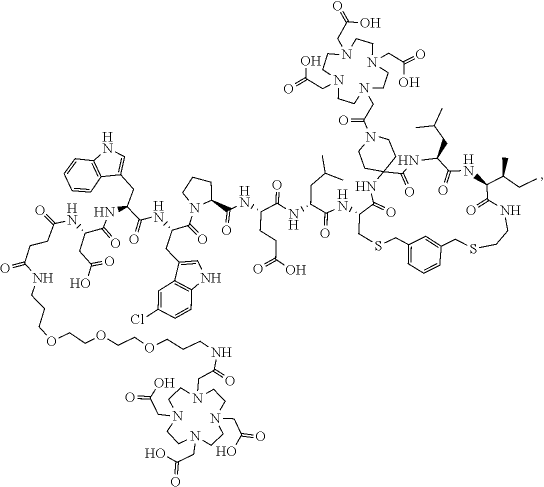 [SMILES: CC[C@H](C)[C@@H]1NC(=O)[C@H](CC(C)C)NC(=O)C2(CCN(C(=O)CN3CCN(CC(=O)O)CCN(CC(=O)O)CCN(CC(=O)O)CC3)CC2)NC(=O)[C@@H](NC(=O)[C@@H](CC(C)C)NC(=O)[C@H](CCC(=O)O)NC(=O)[C@@H]2CCCN2C(=O)[C@H](Cc2c[nH]c3ccc(Cl)cc23)NC(=O)[C@H](Cc2c[nH]c3ccccc23)NC(=O)[C@H](CC(=O)O)NC(=O)CCC(=O)NCCCOCCOCCOCCCNC(=O)CN2CCN(CC(=O)O)CCN(CC(=O)O)CCN(CC(=O)O)CC2)CSCc2cccc(c2)CSCCNC1=O]
compound DOTA-Ttds-Asp-Trp-5Clw-Hyp-Glu-leu-[Cys(2Lut)-Thp-Leu-Ile-Cys]-Lys(DOTA)-NH₂ (GIP-054) of the following formula 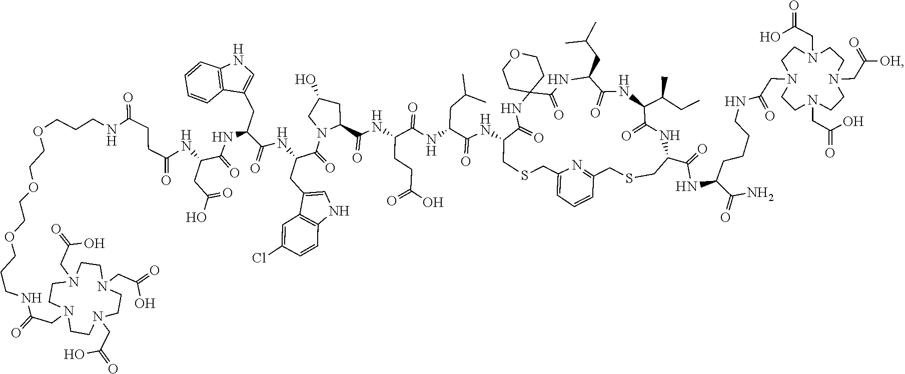 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CC(=O)O)NC(=O)CCC(=O)NCCCOCCOCCOCCCNC(=O)CN2CCN(CC(=O)O)CCN(CC(=O)O)CCN(CC(=O)O)CC2)CSCc2cccc(n2)CSC[C@@H](C(=O)N[C@@H](CCCCNC(=O)CN2CCN(CC(=O)O)CCN(CC(=O)O)CCN(CC(=O)O)CC2)C(N)=O)NC1=O]
compound DOTA-Ttds-Asp-Trp-5Clw-Hyp-Glu-leu-[Cys(2Lut)-Apc(DOTA)-Leu-Ile-Cys]-Lys(DOTA)-NH₂ (GIP-055) of the following formula 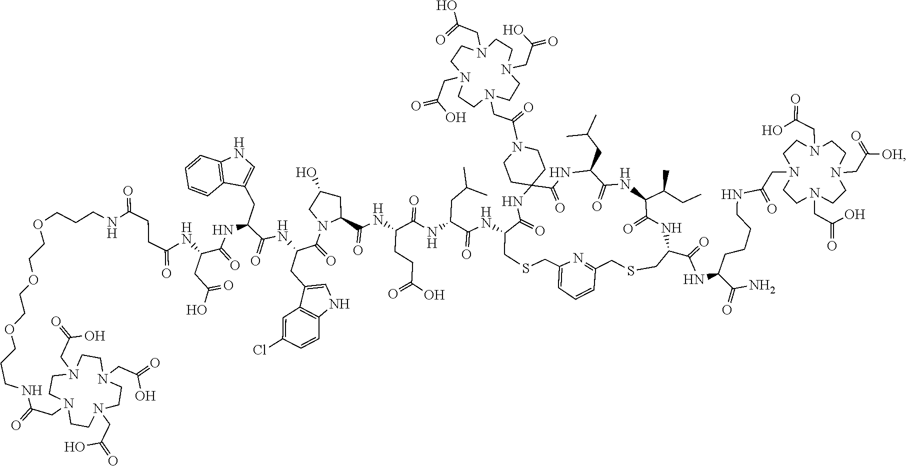 [SMILES: CC[C@H](C)[C@@H]1NC(=O)[C@H](CC(C)C)NC(=O)C2(CCN(C(=O)CN3CCN(CC(=O)O)CCN(CC(=O)O)CCN(CC(=O)O)CC3)CC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CC(=O)O)NC(=O)CCC(=O)NCCCOCCOCCOCCCNC(=O)CN2CCN(CC(=O)O)CCN(CC(=O)O)CCN(CC(=O)O)CC2)CSCc2cccc(n2)CSC[C@@H](C(=O)N[C@@H](CCCCNC(=O)CN2CCN(CC(=O)O)CCN(CC(=O)O)CCN(CC(=O)O)CC2)C(N)=O)NC1=O]
compound DOTA-Ttds-Asp-Trp-5Clw-Tap(DOTA)-Glu-leu-[Cys(2Lut)-Apc(DOTA)-Leu-Ile-Cys]-Lys(DOTA)-NH₂ (GIP-056) of the following formula 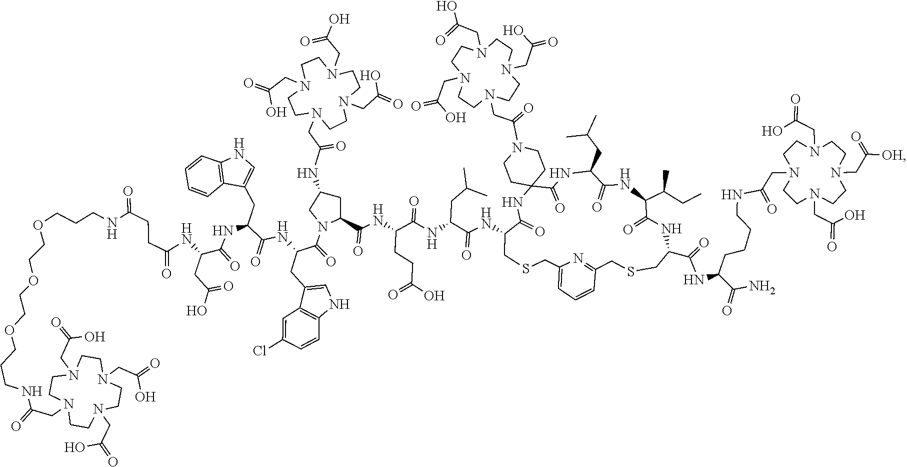 [SMILES: CC[C@H](C)[C@@H]1NC(=O)[C@H](CC(C)C)NC(=O)C2(CCN(C(=O)CN3CCN(CC(=O)O)CCN(CC(=O)O)CCN(CC(=O)O)CC3)CC2)NC(=O)[C@@H](NC(=O)[C@@H](CC(C)C)NC(=O)[C@H](CCC(=O)O)NC(=O)[C@@H]2C[C@@H](NC(=O)CN3CCN(CC(=O)O)CCN(CC(=O)O)CCN(CC(=O)O)CC3)CN2C(=O)[C@H](Cc2c[nH]c3ccc(Cl)cc23)NC(=O)[C@H](Cc2c[nH]c3ccccc23)NC(=O)[C@H](CC(=O)O)NC(=O)CCC(=O)NCCCOCCOCCOCCCNC(=O)CN2CCN(CC(=O)O)CCN(CC(=O)O)CCN(CC(=O)O)CC2)CSCc2cccc(n2)CSC[C@@H](C(=O)N[C@@H](CCCCNC(=O)CN2CCN(CC(=O)O)CCN(CC(=O)O)CCN(CC(=O)O)CC2)C(N)=O)NC1=O]
compound AGLU-Glutar-APAc-Asp-Trp-5Clw-Tap-Glu-leu-[Cys(2Lut)-Apc(GaDOTA-glu(AGLU))-Leu-Ile-Cys]-APAc-glu(AGLU)-NH₂ (GIP-059) of the following formula 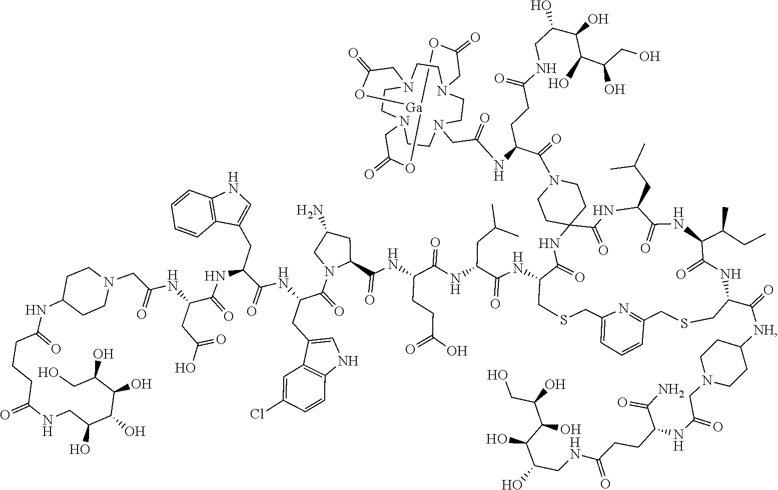
compound AGLU-Glutar-APAc-Asp-Trp-5Clw-Tap-Glu-leu-[Cys(2Lut)-Apc(InDOTA-glu(AGLU))-Leu-Ile-Cys]-APAc-glu(AGLU)-NH₂ (GIP-060) of the following formula 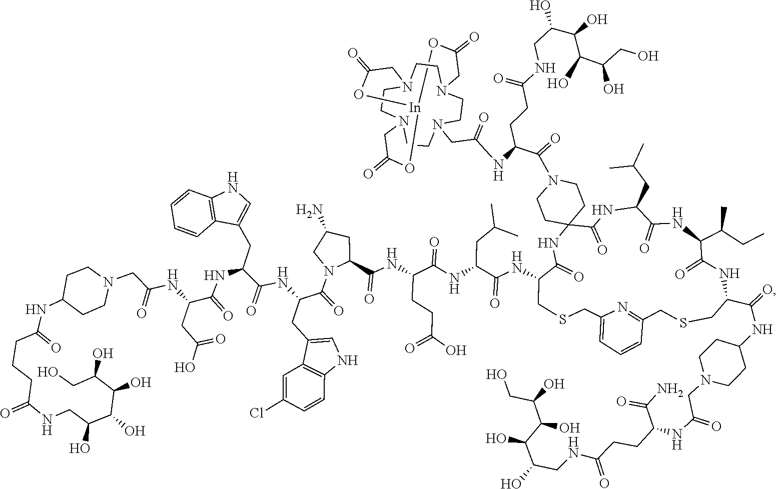
compound AGLU-Glutar-APAc-Asp-Trp-5Clw-Tap-Glu-leu-[Cys(2Lut)-Apc(LuDOTA-glu(AGLU))-Leu-Ile-Cys]-APAc-glu(AGLU)-NH (GIP-061) of the following formula 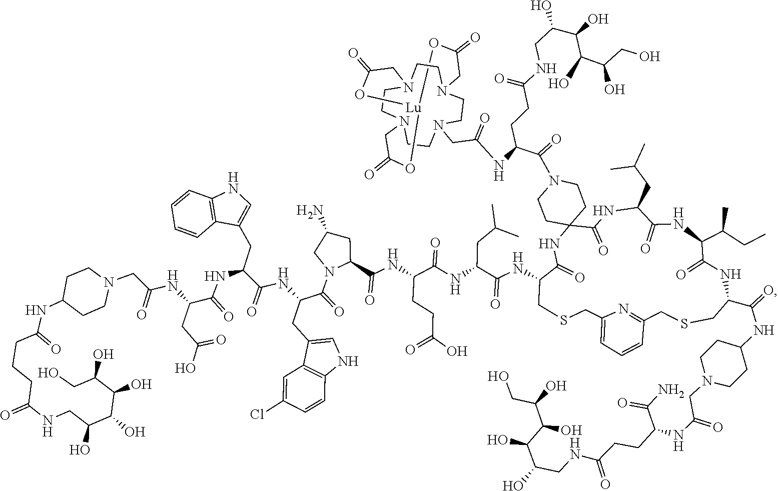
compound LuDOTA-glu(AGLU)-APAc-Asp-Trp-5Clw-Hyp-Glu-leu-[Cys(2Lut)-Thp-Leu-Ile-Cys]-APAc-glu(AGLU)-NH₂ (GIP-062) of the following formula 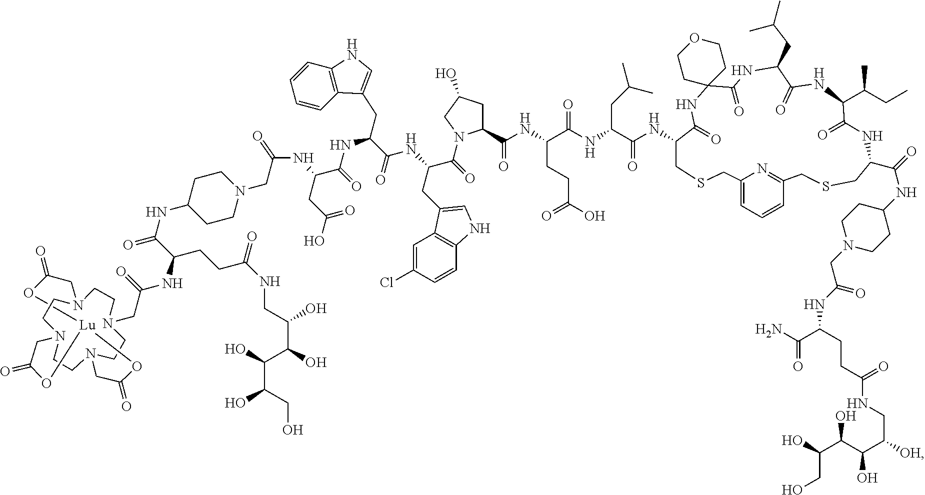
compound InDOTA-glu(AGLU)-APAc-Asp-Trp-5Clw-Hyp-Glu-leu-[Cys(2Lut)-Thp-Leu-Ile-Cys]-APAc-glu(AGLU)-NH₂ (GIP-063) of the following formula 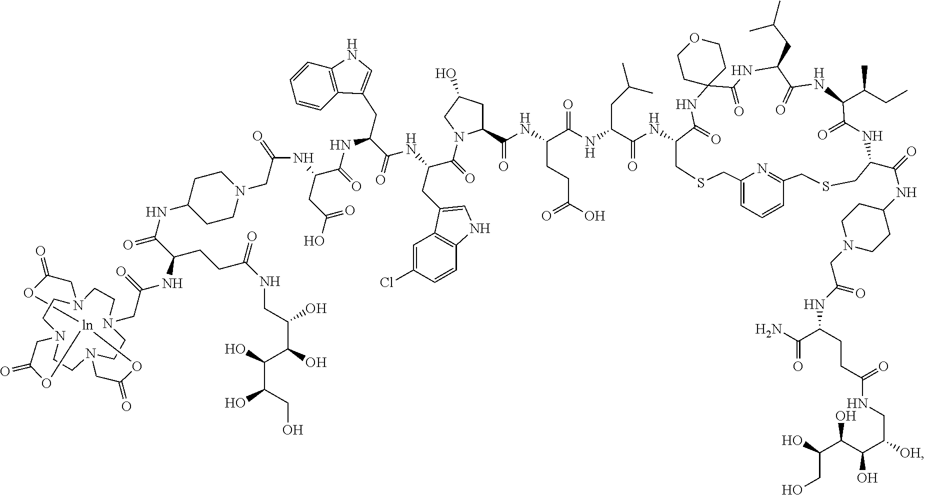
compound EuDOTA-glu(AGLU)-APAc-Asp-Trp-5Clw-Hyp-Glu-leu-[Cys(2Lut)-Thp-Leu-Ile-Cys]-APAc-glu(AGLU)-NH₂ (GIP-064) of the following formula 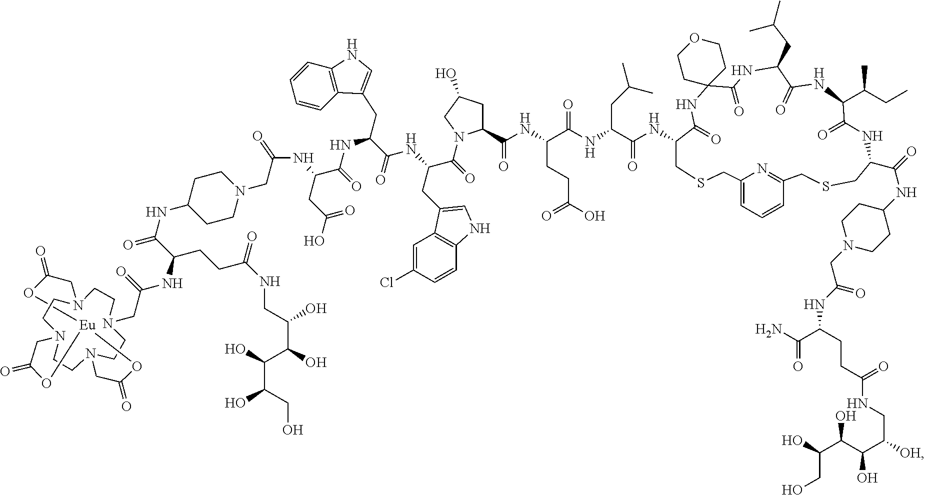
compound AGLU-Glutar-Asp-Trp-5Clw-Hyp-Glu-leu-[Cys(2Lut)-Apc(InDOTA)-Leu-Ile-Cys]-APAc-glu(AGLU)-NH₂ (GIP-065) of the following formula 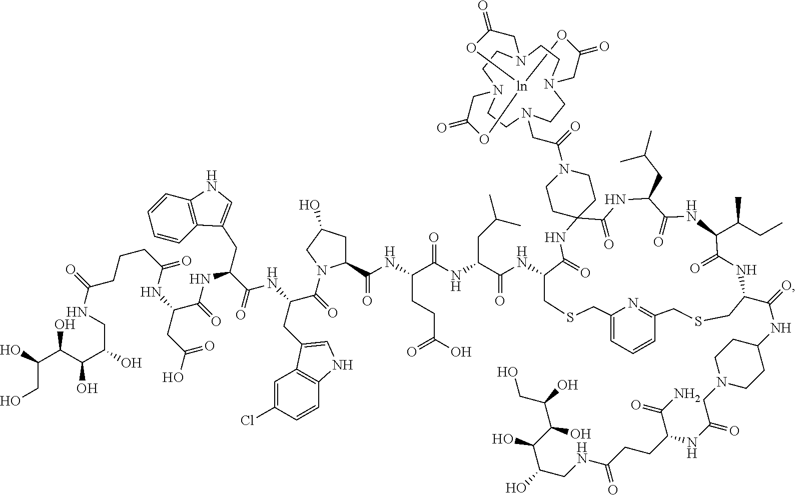
compound AGLU-Glutar-Asp-Trp-5Clw-Hyp-Glu-leu-[Cys(2Lut)-Apc(LuDOTA)-Leu-Ile-Cys]-APAc-glu(AGLU)-NH₂ (GIP-066) of the following formula 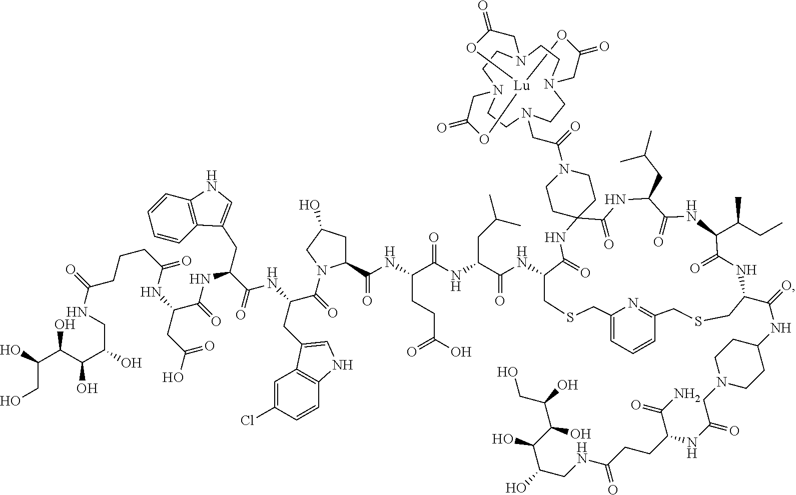
compound AGLU-Glutar-Asp-Trp-5Clw-Tap-Glu-leu-[Cys(2Lut)-Apc(LuDOTA)-Leu-Ile-Cys]-APAc-glu(AGLU)-NH₂ (GIP-067) of the following formula 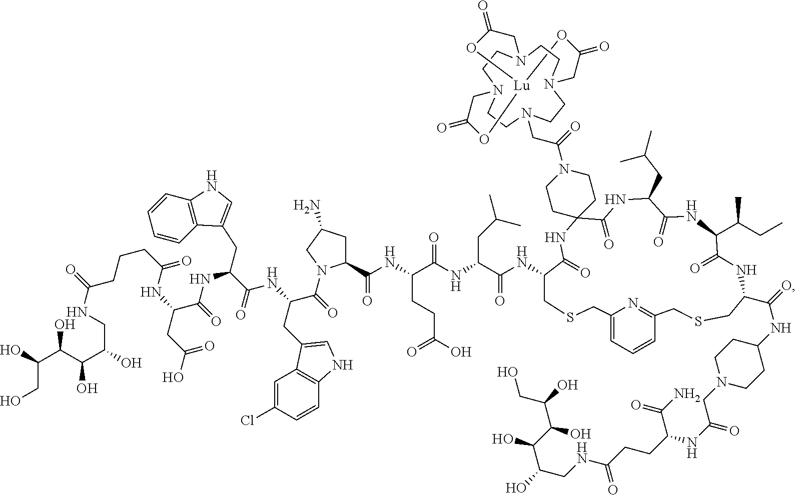
compound AGLU-Glutar-Asp-Trp-5Clw-Tap-Glu-leu-[Cys(2Lut)-Apc(InDOTA)-Leu-Ile-Cys]-APAc-glu(AGLU)-NH₂ (GIP-068) of the following formula 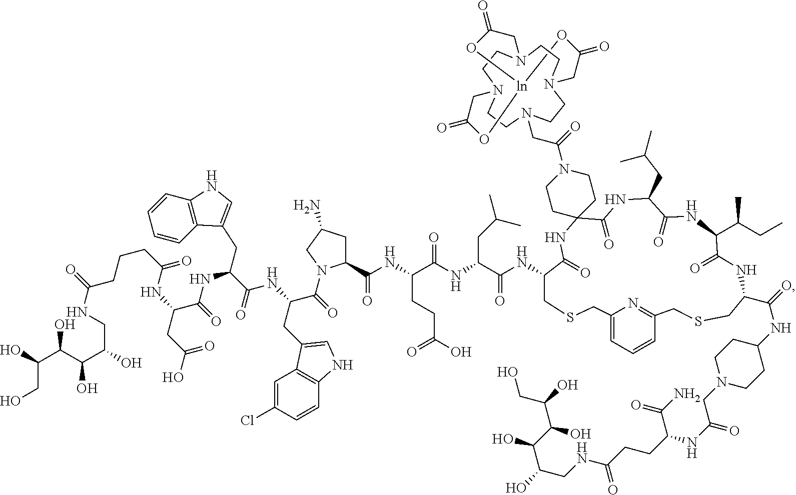
compound AGLU-Glutar-Asp-Trp-5Clw-Tap-Glu-leu-[Cys(2Lut)-Apc(GaDOTA)-Leu-Ile-Cys]-APAc-glu(AGLU)-NH₂ (GIP-069) of the following formula 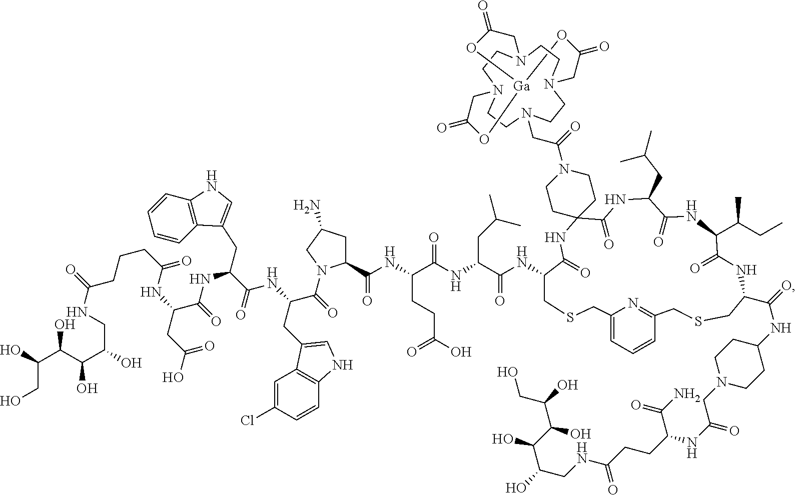
compound AGLU-Glutar-Asp-Trp-5Clw-Hyp-Glu-leu-[Cys(2Lut)-Apc(GaDOTA)-Leu-Ile-Cys]-APAc-glu(AGLU)-NH₂ (GIP-070) of the following formula 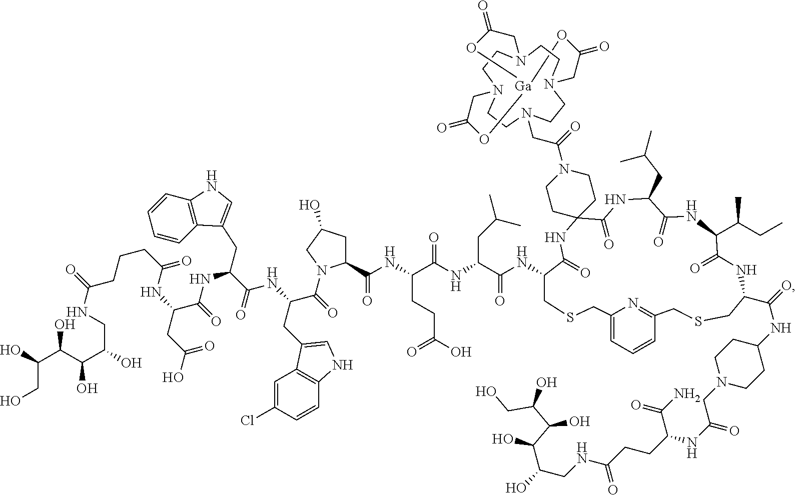
compound AGLU-Glutar-Asp-Trp-5Clw-Tap-Glu-leu-[Cys(2Lut)-Apc(GaNODAGA)-Leu-Ile-Cys]-APAc-glu(AGLU)-NH₂ (GIP-071) of the following formula 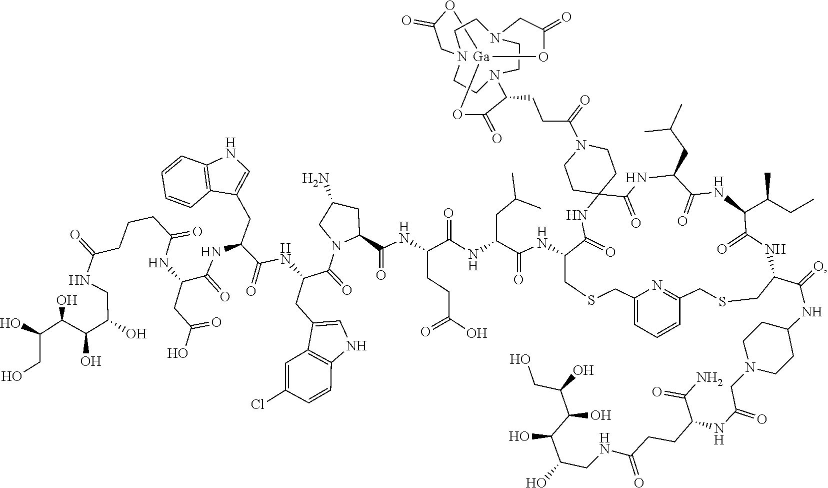
compound AGLU-Glutar-Asp-Trp-5Clw-Hyp-Glu-leu-[Cys(2Lut)-Apc(DOTA)-Leu-Ile-AET] (GIP-072) of the following formula 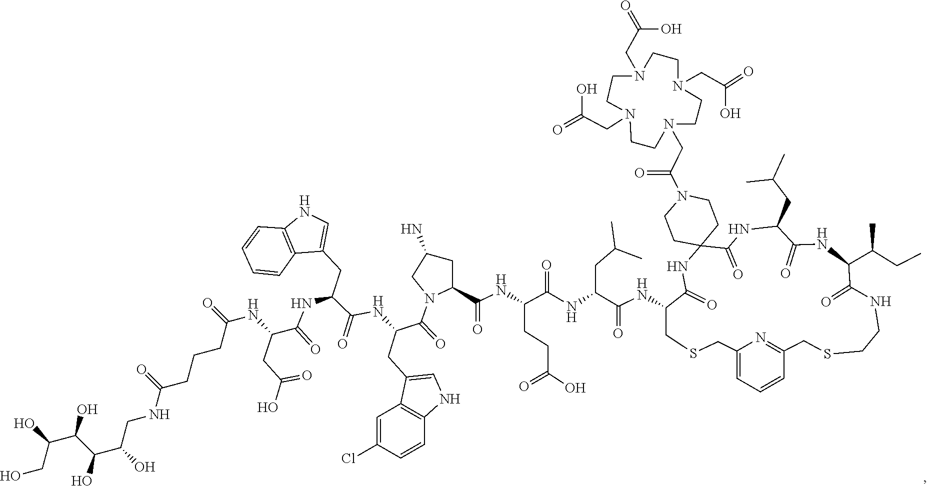 [SMILES: CC[C@H](C)[C@@H]1NC(=O)[C@H](CC(C)C)NC(=O)C2(CCN(C(=O)CN3CCN(CC(=O)O)CCN(CC(=O)O)CCN(CC(=O)O)CC3)CC2)NC(=O)[C@@H](NC(=O)[C@@H](CC(C)C)NC(=O)[C@H](CCC(=O)O)NC(=O)[C@@H]2C[C@@H](N)CN2C(=O)[C@H](Cc2c[nH]c3ccc(Cl)cc23)NC(=O)[C@H](Cc2c[nH]c3ccccc23)NC(=O)[C@H](CC(=O)O)NC(=O)CCCC(=O)NC[C@H](O)[C@@H](O)[C@H](O)[C@H](O)CO)CSCc2cccc(n2)CSCCNC1=O]
compound HO-Succinyl-Trp-5Clw-Hyp-Glu-leu-[Cys(2Lut)-Apc(AGLU-Glutar)-Leu-Ile-Cys]-APAc-Lys(DOTA)-NH₂ (GIP-073) of the following formula 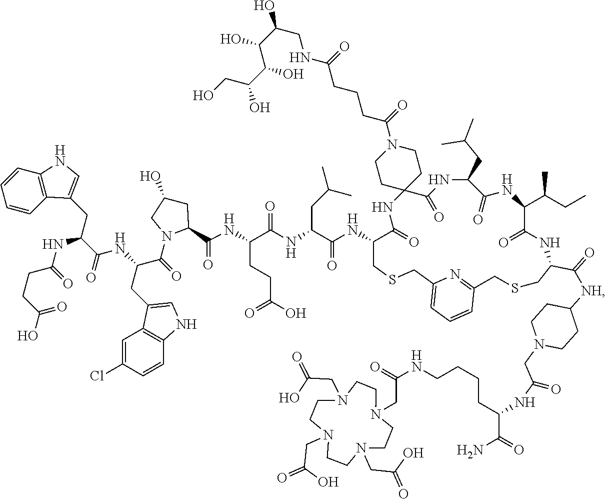 [SMILES: CC[C@H](C)[C@@H]1NC(=O)[C@H](CC(C)C)NC(=O)C2(CCN(C(=O)CCCC(=O)NC[C@H](O)[C@@H](O)[C@H](O)[C@H](O)CO)CC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)CCC(=O)O)CSCc2cccc(n2)CSC[C@@H](C(=O)NC2CCN(CC(=O)N[C@@H](CCCCNC(=O)CN3CCN(CC(=O)O)CCN(CC(=O)O)CCN(CC(=O)O)CC3)C(N)=O)CC2)NC1=O]
compound Ac-Asp-Trp-1Ni-Pro-Glu-Leu-[Cys(3MeBn)-Aib-Leu-Ile-Cys]-Ttds-Lys(DOTA)-NH₂ (GIP-074) of the following formula 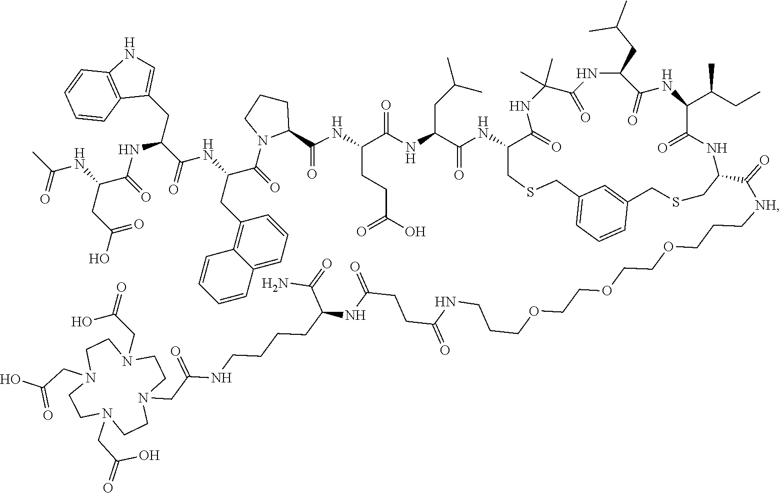 [SMILES: CC[C@H](C)[C@@H]1NC(=O)[C@H](CC(C)C)NC(=O)C(C)(C)NC(=O)[C@@H](NC(=O)[C@H](CC(C)C)NC(=O)[C@H](CCC(=O)O)NC(=O)[C@@H]2CCCN2C(=O)[C@H](Cc2cccc3ccccc23)NC(=O)[C@H](Cc2c[nH]c3ccccc23)NC(=O)[C@H](CC(=O)O)NC(C)=O)CSCc2cccc(c2)CSC[C@@H](C(=O)NCCCOCCOCCOCCCNC(=O)CCC(=O)N[C@@H](CCCCNC(=O)CN2CCN(CC(=O)O)CCN(CC(=O)O)CCN(CC(=O)O)CC2)C(N)=O)NC1=O]
compound Ac-Asp-Trp-Bta-Pro-Glu-Leu-[Cys(3MeBn)-Aib-Leu-Ile-Cys]-Ttds-Lys(DOTA)-NH₂ (GIP-075) of the following formula 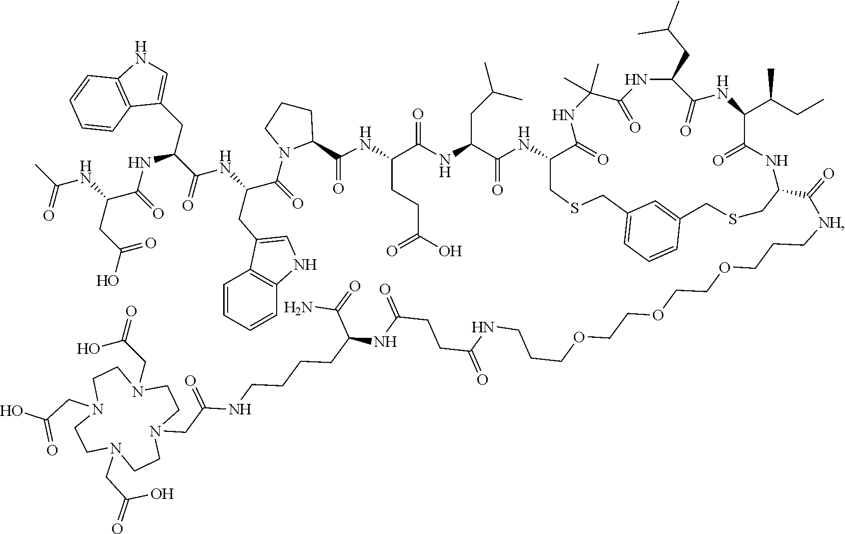 [SMILES: CC[C@H](C)[C@@H]1NC(=O)[C@H](CC(C)C)NC(=O)C(C)(C)NC(=O)[C@@H](NC(=O)[C@H](CC(C)C)NC(=O)[C@H](CCC(=O)O)NC(=O)[C@@H]2CCCN2C(=O)[C@H](Cc2c[nH]c3ccccc23)NC(=O)[C@H](Cc2c[nH]c3ccccc23)NC(=O)[C@H](CC(=O)O)NC(C)=O)CSCc2cccc(c2)CSC[C@@H](C(=O)NCCCOCCOCCOCCCNC(=O)CCC(=O)N[C@@H](CCCCNC(=O)CN2CCN(CC(=O)O)CCN(CC(=O)O)CCN(CC(=O)O)CC2)C(N)=O)NC1=O]
compound Ac-Asp-Trp-5Clw-Pro-Glu-Leu-[Cys(3MeBn)-Aib-Leu-Ile-Cys]-Ttds-Lys(DOTA)-NH₂ (GIP-076) of the following formula 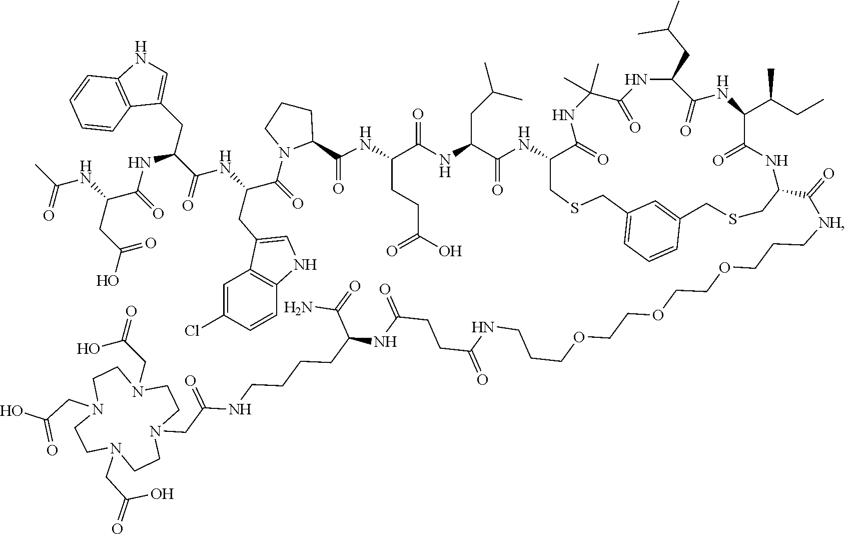 [SMILES: CC[C@H](C)[C@@H]1NC(=O)[C@H](CC(C)C)NC(=O)C(C)(C)NC(=O)[C@@H](NC(=O)[C@H](CC(C)C)NC(=O)[C@H](CCC(=O)O)NC(=O)[C@@H]2CCCN2C(=O)[C@H](Cc2c[nH]c3ccc(Cl)cc23)NC(=O)[C@H](Cc2c[nH]c3ccccc23)NC(=O)[C@H](CC(=O)O)NC(C)=O)CSCc2cccc(c2)CSC[C@@H](C(=O)NCCCOCCOCCOCCCNC(=O)CCC(=O)N[C@@H](CCCCNC(=O)CN2CCN(CC(=O)O)CCN(CC(=O)O)CCN(CC(=O)O)CC2)C(N)=O)NC1=O]
compound DOTA-Ttds-Asp-Trp-5Clw-Pro-Glu-leu-[Cys(3MeBn)-Aib-Leu-Ile-AET] (GIP-077) of the following formula 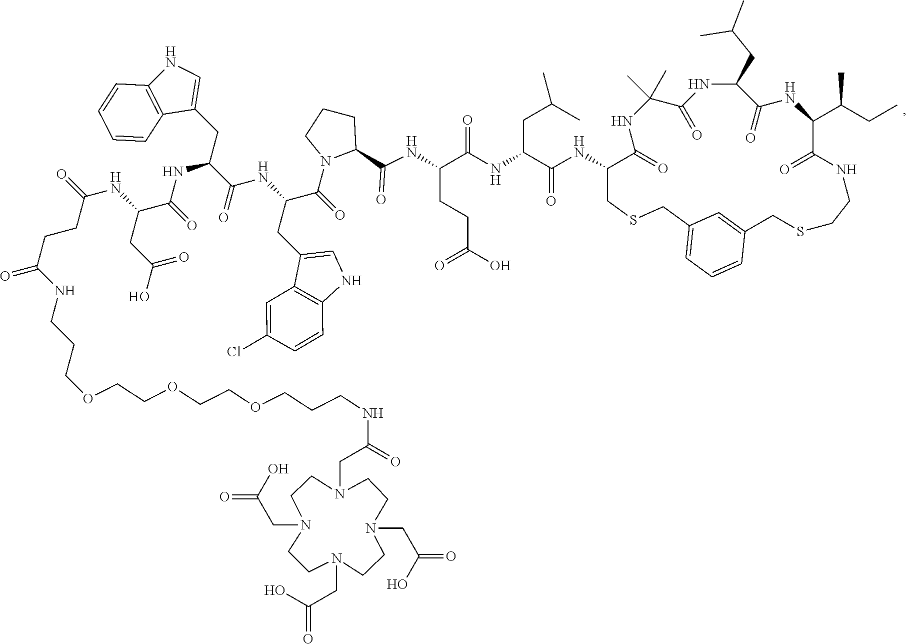 [SMILES: CC[C@H](C)[C@@H]1NC(=O)[C@H](CC(C)C)NC(=O)C(C)(C)NC(=O)[C@@H](NC(=O)[C@@H](CC(C)C)NC(=O)[C@H](CCC(=O)O)NC(=O)[C@@H]2CCCN2C(=O)[C@H](Cc2c[nH]c3ccc(Cl)cc23)NC(=O)[C@H](Cc2c[nH]c3ccccc23)NC(=O)[C@H](CC(=O)O)NC(=O)CCC(=O)NCCCOCCOCCOCCCNC(=O)CN2CCN(CC(=O)O)CCN(CC(=O)O)CCN(CC(=O)O)CC2)CSCc2cccc(c2)CSCCNC1=O]
compound Ac-Asp-Trp-1Ni-Pro-Glu-leu-[Cys(3MeBn)-Aib-Leu-Ile-Cys]-Ttds-Lys(DOTA)-NH₂ (GIP-078) of the following formula 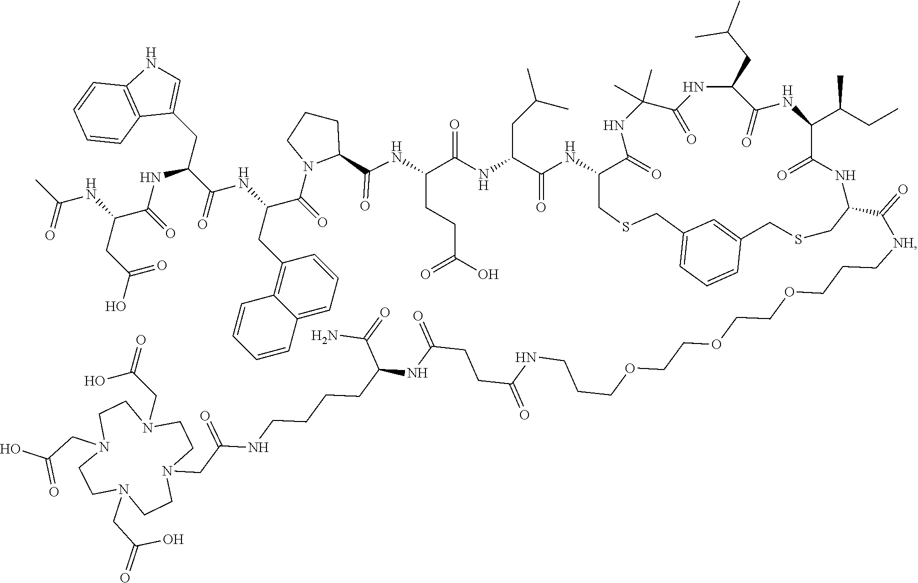 [SMILES: CC[C@H](C)[C@@H]1NC(=O)[C@H](CC(C)C)NC(=O)C(C)(C)NC(=O)[C@@H](NC(=O)[C@@H](CC(C)C)NC(=O)[C@H](CCC(=O)O)NC(=O)[C@@H]2CCCN2C(=O)[C@H](Cc2cccc3ccccc23)NC(=O)[C@H](Cc2c[nH]c3ccccc23)NC(=O)[C@H](CC(=O)O)NC(C)=O)CSCc2cccc(c2)CSC[C@@H](C(=O)NCCCOCCOCCOCCCNC(=O)CCC(=O)N[C@@H](CCCCNC(=O)CN2CCN(CC(=O)O)CCN(CC(=O)O)CCN(CC(=O)O)CC2)C(N)=O)NC1=O]
compound Ac-Asp-Trp-5Clw-Pro-Glu-leu-[Cys(3MeBn)-Aib-Leu-Ile-Cys]-Ttds-Lys(DOTA)-NH₂ (GIP-079) of the following formula 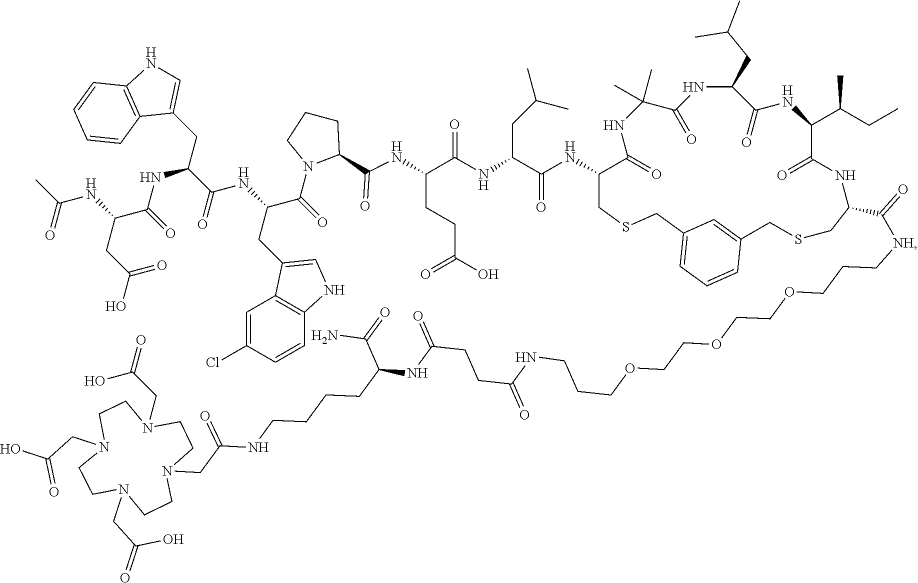 [SMILES: CC[C@H](C)[C@@H]1NC(=O)[C@H](CC(C)C)NC(=O)C(C)(C)NC(=O)[C@@H](NC(=O)[C@@H](CC(C)C)NC(=O)[C@H](CCC(=O)O)NC(=O)[C@@H]2CCCN2C(=O)[C@H](Cc2c[nH]c3ccc(Cl)cc23)NC(=O)[C@H](Cc2c[nH]c3ccccc23)NC(=O)[C@H](CC(=O)O)NC(C)=O)CSCc2cccc(c2)CSC[C@@H](C(=O)NCCCOCCOCCOCCCNC(=O)CCC(=O)N[C@@H](CCCCNC(=O)CN2CCN(CC(=O)O)CCN(CC(=O)O)CCN(CC(=O)O)CC2)C(N)=O)NC1=O]
compound Ac-Asp-Trp-1Ni-Pro-Glu-leu-[Cys(3MeBn)-Egz-Leu-Ile-Cys]-Ttds-Lys(DOTA)-NH₂ (GIP-080) of the following formula 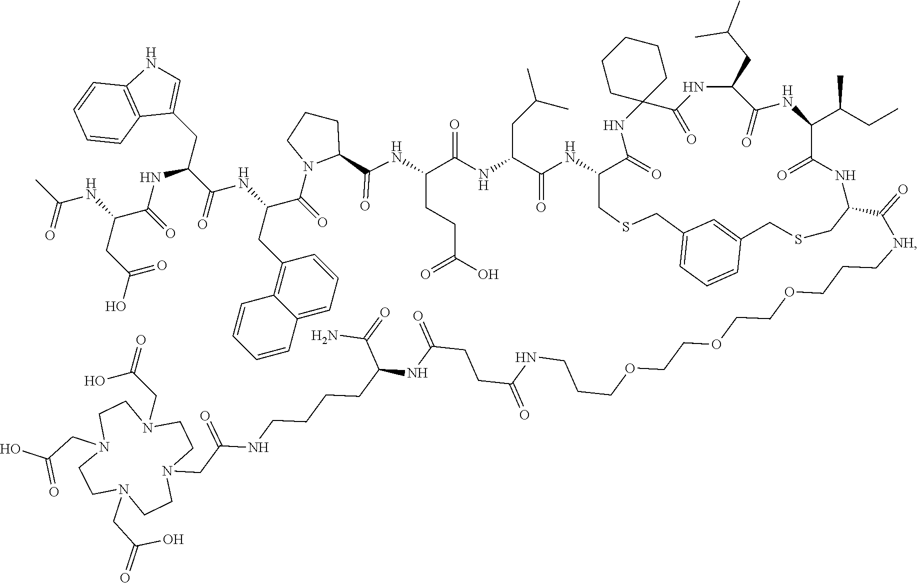 [SMILES: CC[C@H](C)[C@@H]1NC(=O)[C@H](CC(C)C)NC(=O)C2(CCCCC2)NC(=O)[C@@H](NC(=O)[C@@H](CC(C)C)NC(=O)[C@H](CCC(=O)O)NC(=O)[C@@H]2CCCN2C(=O)[C@H](Cc2cccc3ccccc23)NC(=O)[C@H](Cc2c[nH]c3ccccc23)NC(=O)[C@H](CC(=O)O)NC(C)=O)CSCc2cccc(c2)CSC[C@@H](C(=O)NCCCOCCOCCOCCCNC(=O)CCC(=O)N[C@@H](CCCCNC(=O)CN2CCN(CC(=O)O)CCN(CC(=O)O)CCN(CC(=O)O)CC2)C(N)=O)NC1=O]
compound Ac-Asp-Trp-5Fw-Pro-Glu-leu-[Cys(3MeBn)-Aib-Leu-Ile-Cys]-Ttds-Lys(DOTA)-NH₂ (GIP-081) of the following formula 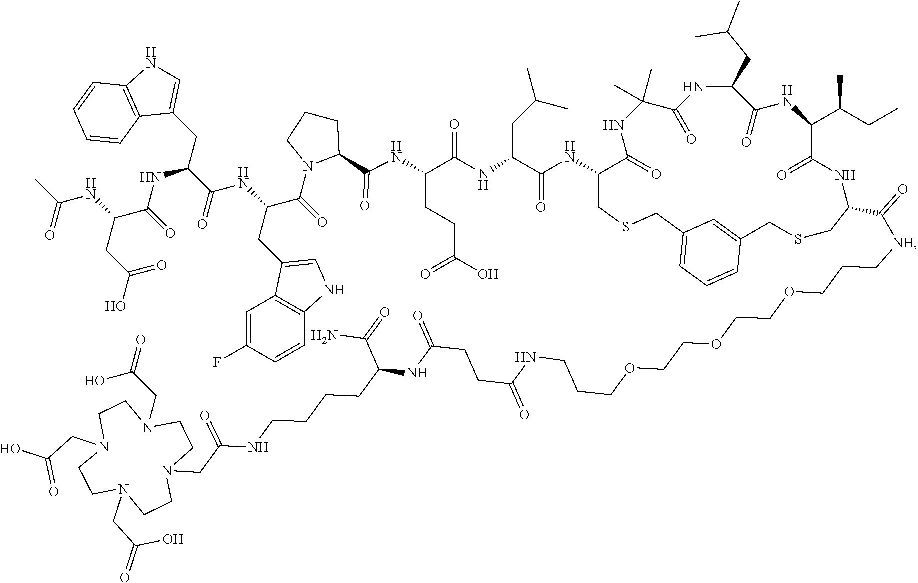 [SMILES: CC[C@H](C)[C@@H]1NC(=O)[C@H](CC(C)C)NC(=O)C(C)(C)NC(=O)[C@@H](NC(=O)[C@@H](CC(C)C)NC(=O)[C@H](CCC(=O)O)NC(=O)[C@@H]2CCCN2C(=O)[C@H](Cc2c[nH]c3ccc(F)cc23)NC(=O)[C@H](Cc2c[nH]c3ccccc23)NC(=O)[C@H](CC(=O)O)NC(C)=O)CSCc2cccc(c2)CSC[C@@H](C(=O)NCCCOCCOCCOCCCNC(=O)CCC(=O)N[C@@H](CCCCNC(=O)CN2CCN(CC(=O)O)CCN(CC(=O)O)CCN(CC(=O)O)CC2)C(N)=O)NC1=O]
compound Ac-Asp-Trp-5Brw-Pro-Glu-leu-[Cys(3MeBn)-Aib-Leu-Ile-Cys]-Ttds-Lys(DOTA)-NH₂ (GIP-082) of the following formula 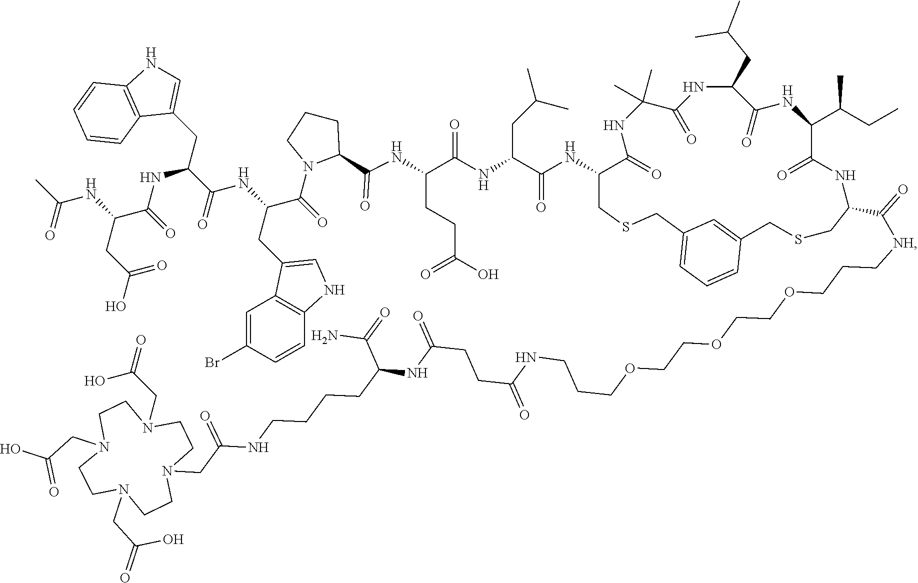 [SMILES: CC[C@H](C)[C@@H]1NC(=O)[C@H](CC(C)C)NC(=O)C(C)(C)NC(=O)[C@@H](NC(=O)[C@@H](CC(C)C)NC(=O)[C@H](CCC(=O)O)NC(=O)[C@@H]2CCCN2C(=O)[C@H](Cc2c[nH]c3ccc(Br)cc23)NC(=O)[C@H](Cc2c[nH]c3ccccc23)NC(=O)[C@H](CC(=O)O)NC(C)=O)CSCc2cccc(c2)CSC[C@@H](C(=O)NCCCOCCOCCOCCCNC(=O)CCC(=O)N[C@@H](CCCCNC(=O)CN2CCN(CC(=O)O)CCN(CC(=O)O)CCN(CC(=O)O)CC2)C(N)=O)NC1=O]
compound Ac-Asp-5Fw-1Ni-Pro-Glu-leu-[Cys(3MeBn)-Aib-Leu-Ile-Cys]-Ttds-Lys(DOTA)-NH₂ (GIP-083) of the following formula 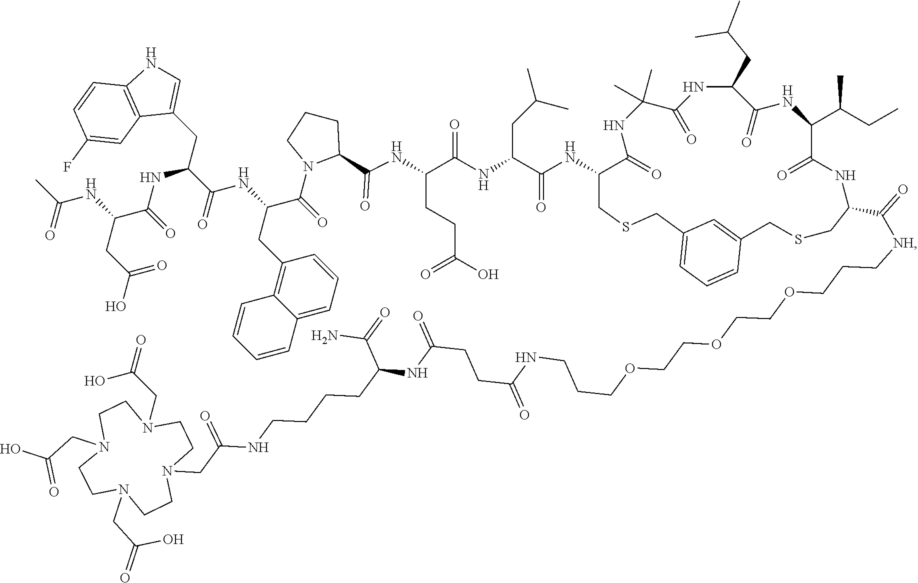 [SMILES: CC[C@H](C)[C@@H]1NC(=O)[C@H](CC(C)C)NC(=O)C(C)(C)NC(=O)[C@@H](NC(=O)[C@@H](CC(C)C)NC(=O)[C@H](CCC(=O)O)NC(=O)[C@@H]2CCCN2C(=O)[C@H](Cc2cccc3ccccc23)NC(=O)[C@H](Cc2c[nH]c3ccc(F)cc23)NC(=O)[C@H](CC(=O)O)NC(C)=O)CSCc2cccc(c2)CSC[C@@H](C(=O)NCCCOCCOCCOCCCNC(=O)CCC(=O)N[C@@H](CCCCNC(=O)CN2CCN(CC(=O)O)CCN(CC(=O)O)CCN(CC(=O)O)CC2)C(N)=O)NC1=O]
compound Ac-Asp-6Fw-1Ni-Pro-Glu-leu-[Cys(3MeBn)-Aib-Leu-Ile-Cys]-Ttds-Lys(DOTA)-NH₂ (GIP-084) of the following formula 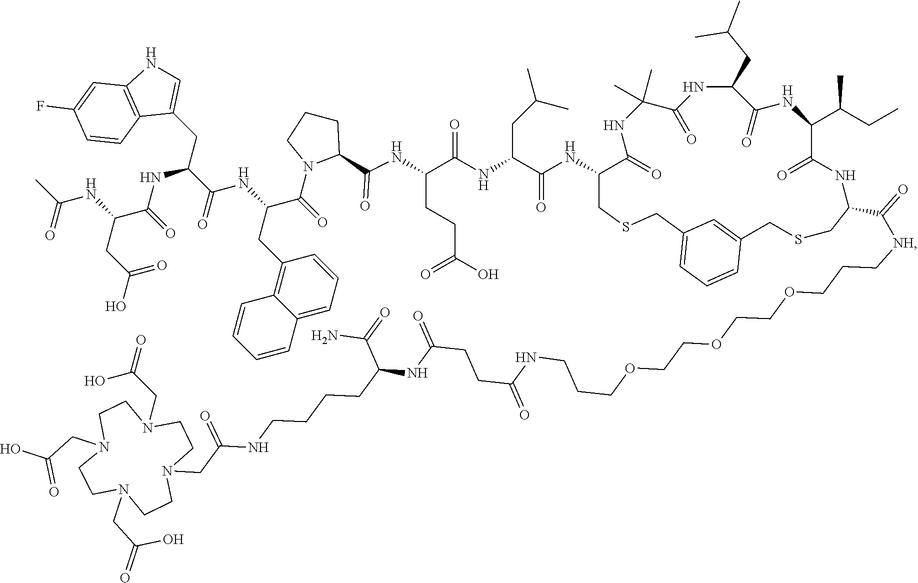 [SMILES: CC[C@H](C)[C@@H]1NC(=O)[C@H](CC(C)C)NC(=O)C(C)(C)NC(=O)[C@@H](NC(=O)[C@@H](CC(C)C)NC(=O)[C@H](CCC(=O)O)NC(=O)[C@@H]2CCCN2C(=O)[C@H](Cc2cccc3ccccc23)NC(=O)[C@H](Cc2c[nH]c3cc(F)ccc23)NC(=O)[C@H](CC(=O)O)NC(C)=O)CSCc2cccc(c2)CSC[C@@H](C(=O)NCCCOCCOCCOCCCNC(=O)CCC(=O)N[C@@H](CCCCNC(=O)CN2CCN(CC(=O)O)CCN(CC(=O)O)CCN(CC(=O)O)CC2)C(N)=O)NC1=O]
compound HO-Succinyl-Trp-1Ni-Pro-Glu-Leu-[Cys(3MeBn)-Aib-Leu-Ile-Cys]-Ttds-Lys(DOTA)-NH₂ (GIP-085) of the following formula 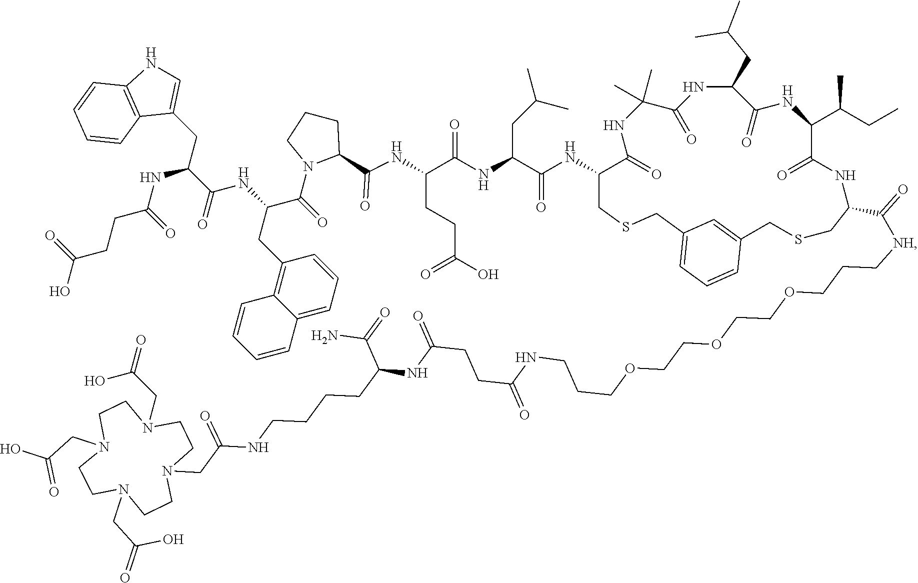 [SMILES: CC[C@H](C)[C@@H]1NC(=O)[C@H](CC(C)C)NC(=O)C(C)(C)NC(=O)[C@@H](NC(=O)[C@H](CC(C)C)NC(=O)[C@H](CCC(=O)O)NC(=O)[C@@H]2CCCN2C(=O)[C@H](Cc2cccc3ccccc23)NC(=O)[C@H](Cc2c[nH]c3ccccc23)NC(=O)CCC(=O)O)CSCc2cccc(c2)CSC[C@@H](C(=O)NCCCOCCOCCOCCCNC(=O)CCC(=O)N[C@@H](CCCCNC(=O)CN2CCN(CC(=O)O)CCN(CC(=O)O)CCN(CC(=O)O)CC2)C(N)=O)NC1=O]
compound DOTA-Ttds-Asp-Trp-5Clw-Pro-Glu-leu-[Cys(3MeBn)-Egz-Leu-Ile-AET] (GIP-086) of the following formula 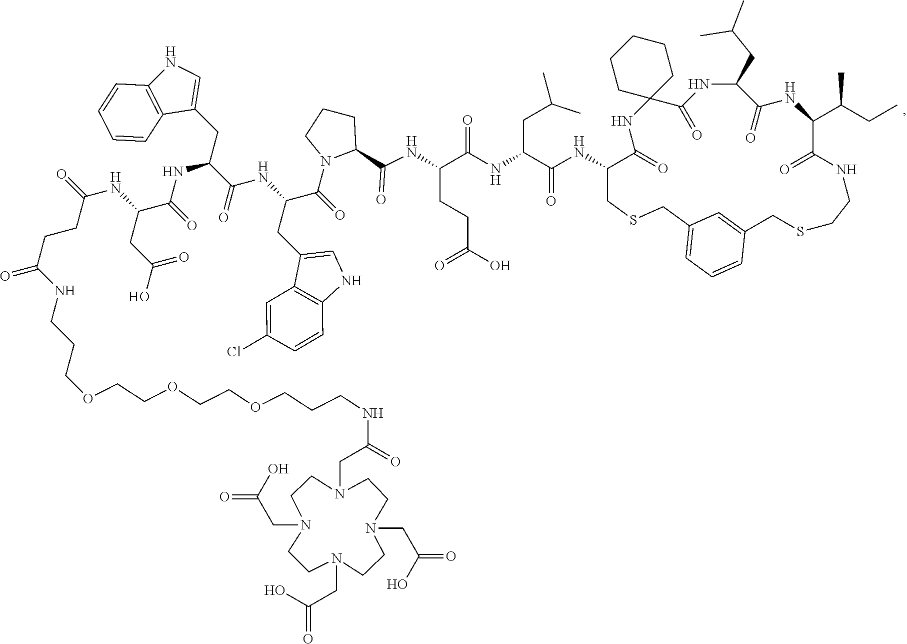 [SMILES: CC[C@H](C)[C@@H]1NC(=O)[C@H](CC(C)C)NC(=O)C2(CCCCC2)NC(=O)[C@@H](NC(=O)[C@@H](CC(C)C)NC(=O)[C@H](CCC(=O)O)NC(=O)[C@@H]2CCCN2C(=O)[C@H](Cc2c[nH]c3ccc(Cl)cc23)NC(=O)[C@H](Cc2c[nH]c3ccccc23)NC(=O)[C@H](CC(=O)O)NC(=O)CCC(=O)NCCCOCCOCCOCCCNC(=O)CN2CCN(CC(=O)O)CCN(CC(=O)O)CCN(CC(=O)O)CC2)CSCc2cccc(c2)CSCCNC1=O]
compound DOTA-Ttds-Asp-Trp-5Clw-Pro-Glu-leu-[Cys(3MeBn)-Thp-Leu-Ile-AET] (GIP-087) of the following formula 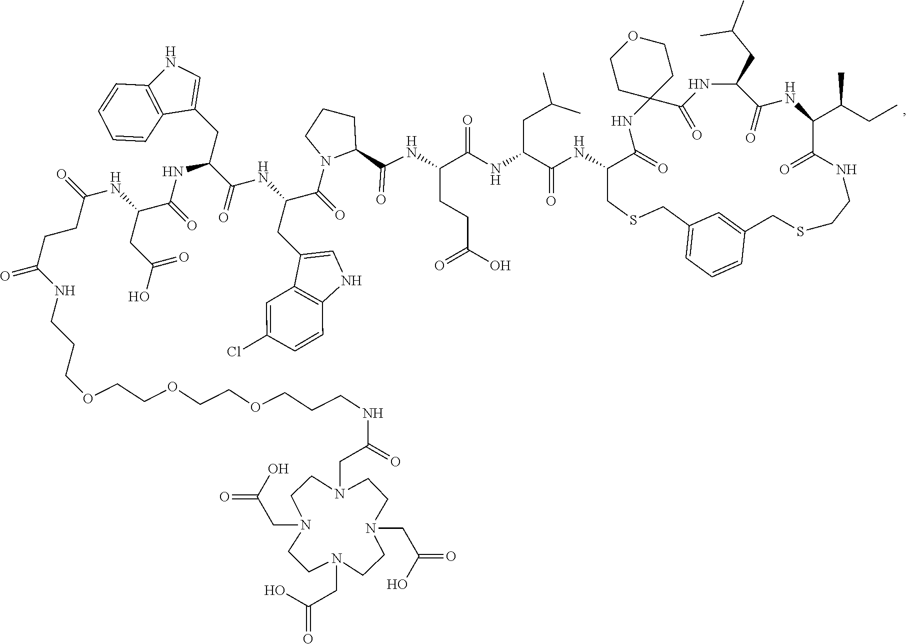 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2CCCN2C(=O)[C@H](Cc2c[nH]c3ccc(Cl)cc23)NC(=O)[C@H](Cc2c[nH]c3ccccc23)NC(=O)[C@H](CC(=O)O)NC(=O)CCC(=O)NCCCOCCOCCOCCCNC(=O)CN2CCN(CC(=O)O)CCN(CC(=O)O)CCN(CC(=O)O)CC2)CSCc2cccc(c2)CSCCNC1=O]
compound DOTA-Ttds-Asp-Trp-5Clw-Pro-Glu-leu-[Cys(3MeBn)-Ahpc-Leu-Ile-AET] (GIP-088) of the following formula 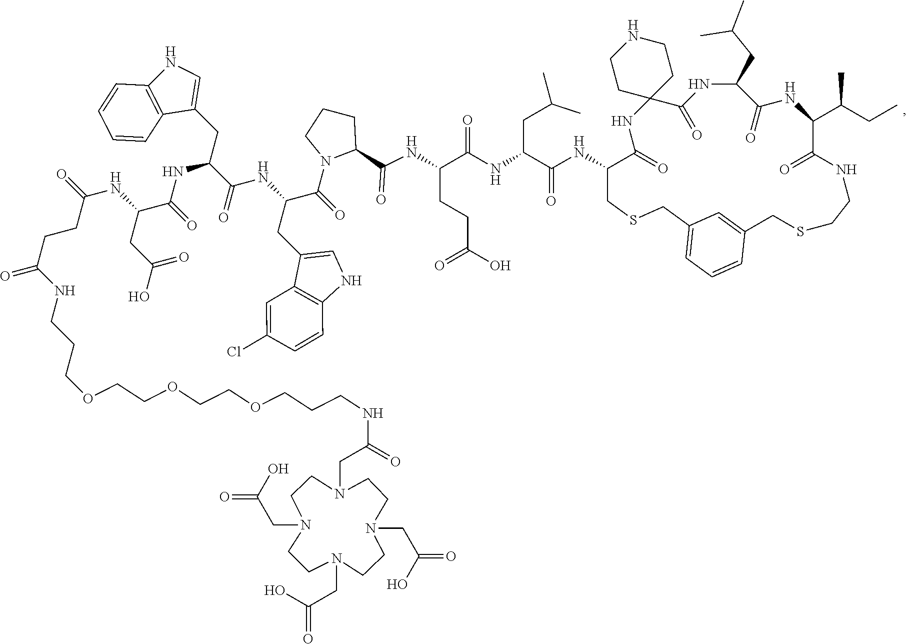 [SMILES: CC[C@H](C)[C@@H]1NC(=O)[C@H](CC(C)C)NC(=O)C2(CCNCC2)NC(=O)[C@@H](NC(=O)[C@@H](CC(C)C)NC(=O)[C@H](CCC(=O)O)NC(=O)[C@@H]2CCCN2C(=O)[C@H](Cc2c[nH]c3ccc(Cl)cc23)NC(=O)[C@H](Cc2c[nH]c3ccccc23)NC(=O)[C@H](CC(=O)O)NC(=O)CCC(=O)NCCCOCCOCCOCCCNC(=O)CN2CCN(CC(=O)O)CCN(CC(=O)O)CCN(CC(=O)O)CC2)CSCc2cccc(c2)CSCCNC1=O]
compound DOTA-APAc-Asp-Trp-5Clw-Pro-Glu-leu-[Cys(3MeBn)-Aib-Leu-Ile-AET] (GIP-089) of the following formula 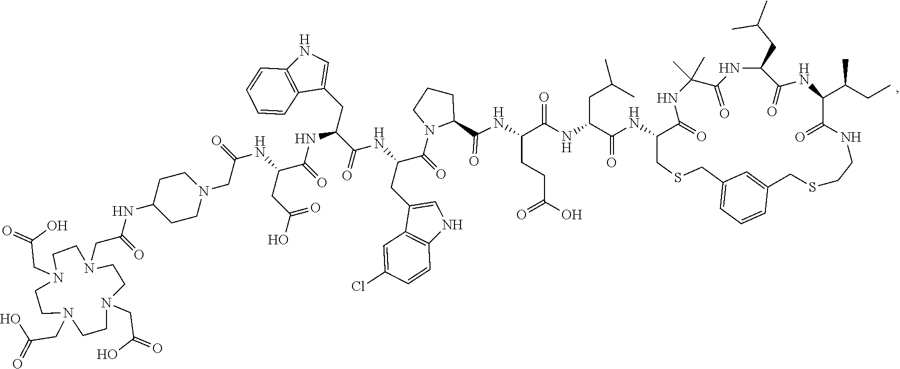 [SMILES: CC[C@H](C)[C@@H]1NC(=O)[C@H](CC(C)C)NC(=O)C(C)(C)NC(=O)[C@@H](NC(=O)[C@@H](CC(C)C)NC(=O)[C@H](CCC(=O)O)NC(=O)[C@@H]2CCCN2C(=O)[C@H](Cc2c[nH]c3ccc(Cl)cc23)NC(=O)[C@H](Cc2c[nH]c3ccccc23)NC(=O)[C@H](CC(=O)O)NC(=O)CN2CCC(NC(=O)CN3CCN(CC(=O)O)CCN(CC(=O)O)CCN(CC(=O)O)CC3)CC2)CSCc2cccc(c2)CSCCNC1=O]
compound DOTA-Ttds-Asp-Trp-5Clw-Pro-Glu-leu-[Cys(2Lut)-Aib-Leu-Ile-AET] (GIP-090) of the following formula 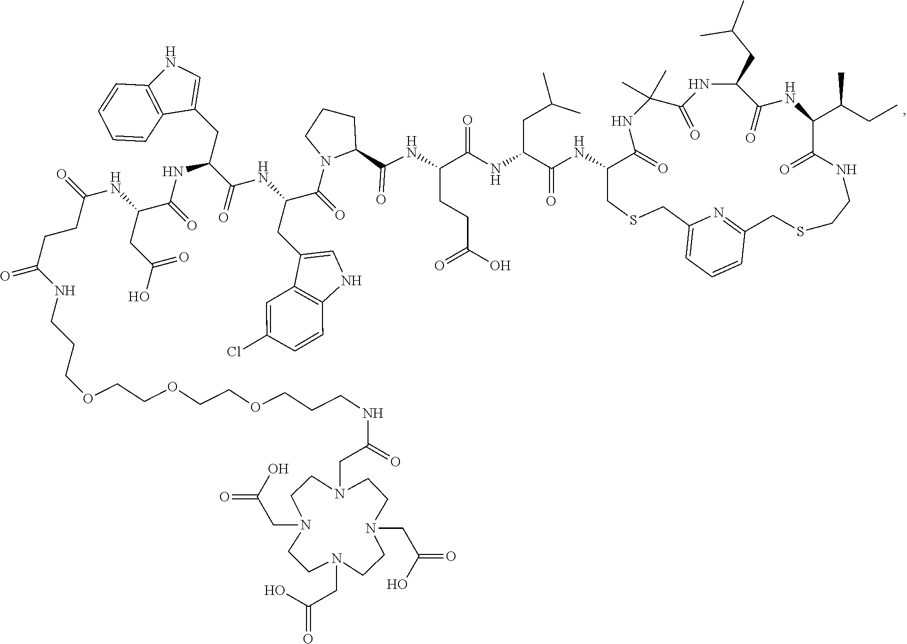 [SMILES: CC[C@H](C)[C@@H]1NC(=O)[C@H](CC(C)C)NC(=O)C(C)(C)NC(=O)[C@@H](NC(=O)[C@@H](CC(C)C)NC(=O)[C@H](CCC(=O)O)NC(=O)[C@@H]2CCCN2C(=O)[C@H](Cc2c[nH]c3ccc(Cl)cc23)NC(=O)[C@H](Cc2c[nH]c3ccccc23)NC(=O)[C@H](CC(=O)O)NC(=O)CCC(=O)NCCCOCCOCCOCCCNC(=O)CN2CCN(CC(=O)O)CCN(CC(=O)O)CCN(CC(=O)O)CC2)CSCc2cccc(n2)CSCCNC1=O]
compound DOTA-Ttds-Asp-Trp-5Clw-Pro-Glu-leu-[Cys(3Lut)-Aib-Leu-Ile-AET] (GIP-091) of the following formula 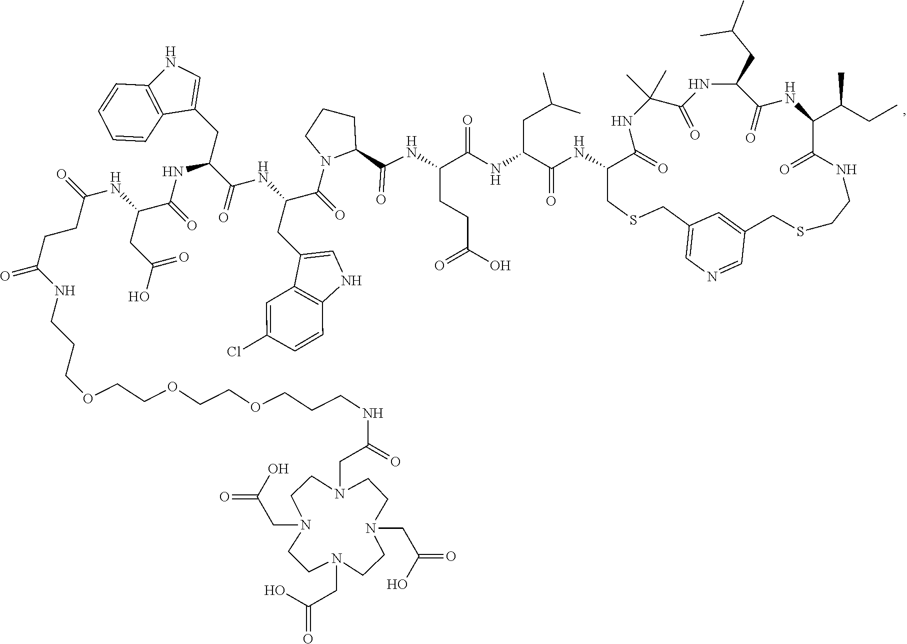 [SMILES: CC[C@H](C)[C@@H]1NC(=O)[C@H](CC(C)C)NC(=O)C(C)(C)NC(=O)[C@@H](NC(=O)[C@@H](CC(C)C)NC(=O)[C@H](CCC(=O)O)NC(=O)[C@@H]2CCCN2C(=O)[C@H](Cc2c[nH]c3ccc(Cl)cc23)NC(=O)[C@H](Cc2c[nH]c3ccccc23)NC(=O)[C@H](CC(=O)O)NC(=O)CCC(=O)NCCCOCCOCCOCCCNC(=O)CN2CCN(CC(=O)O)CCN(CC(=O)O)CCN(CC(=O)O)CC2)CSCc2cncc(c2)CSCCNC1=O]
compound DOTA-Ttds-Asp-Trp-5Clw-Pro-Glu-leu-[Dap(3MeBn)-Aib-Leu-Ile-AET] (GIP-092) of the following formula 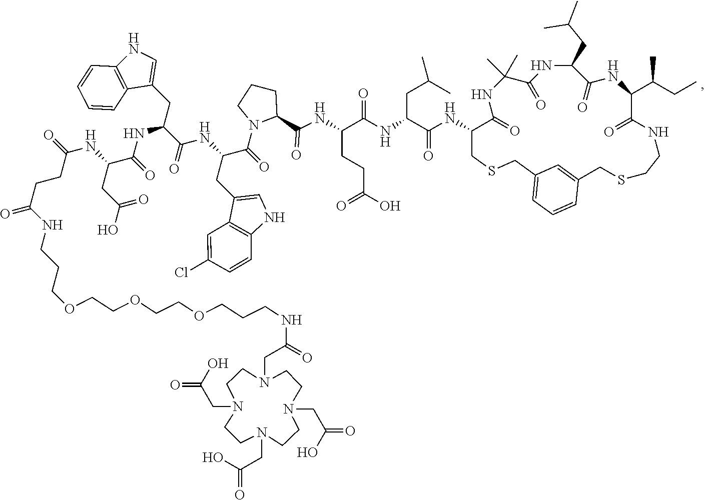 [SMILES: CC[C@H](C)[C@@H]1NC(=O)[C@H](CC(C)C)NC(=O)C(C)(C)NC(=O)[C@@H](NC(=O)[C@@H](CC(C)C)NC(=O)[C@H](CCC(=O)O)NC(=O)[C@@H]2CCCN2C(=O)[C@H](Cc2c[nH]c3ccc(Cl)cc23)NC(=O)[C@H](Cc2c[nH]c3ccccc23)NC(=O)[C@H](CC(=O)O)NC(=O)CCC(=O)NCCCOCCOCCOCCCNC(=O)CN2CCN(CC(=O)O)CCN(CC(=O)O)CCN(CC(=O)O)CC2)CSCc2cccc(c2)CSCCNC1=O]
compound DOTA-Ttds-Asp-Trp-5Clw-Pro-Glu-leu-[Cys(3MeBn)-Aib-Leu-Ile-en] (GIP-093) of the following formula 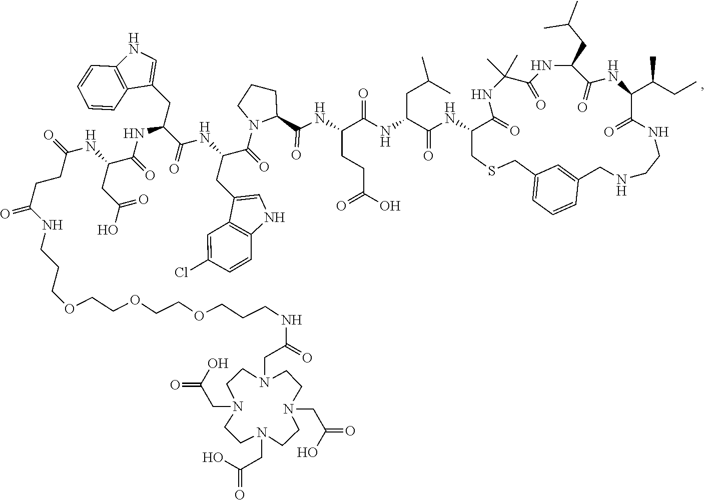 [SMILES: CC[C@H](C)[C@@H]1NC(=O)[C@H](CC(C)C)NC(=O)C(C)(C)NC(=O)[C@@H](NC(=O)[C@@H](CC(C)C)NC(=O)[C@H](CCC(=O)O)NC(=O)[C@@H]2CCCN2C(=O)[C@H](Cc2c[nH]c3ccc(Cl)cc23)NC(=O)[C@H](Cc2c[nH]c3ccccc23)NC(=O)[C@H](CC(=O)O)NC(=O)CCC(=O)NCCCOCCOCCOCCCNC(=O)CN2CCN(CC(=O)O)CCN(CC(=O)O)CCN(CC(=O)O)CC2)CSCc2cccc(c2)CNCCNC1=O]
compound DOTA-Ttds-Asp-Trp-5Clw-Hyp-Glu-leu-[Cys(3MeBn)-Aib-Leu-Ile-AET] (GIP-094) of the following formula 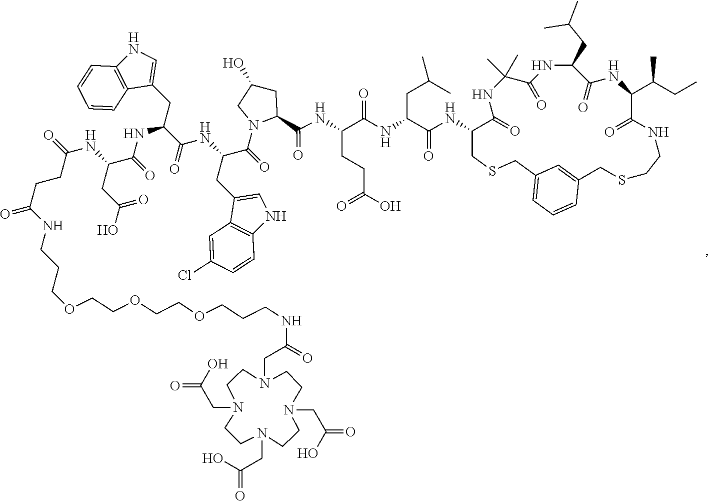 [SMILES: CC[C@H](C)[C@@H]1NC(=O)[C@H](CC(C)C)NC(=O)C(C)(C)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CC(=O)O)NC(=O)CCC(=O)NCCCOCCOCCOCCCNC(=O)CN2CCN(CC(=O)O)CCN(CC(=O)O)CCN(CC(=O)O)CC2)CSCc2cccc(c2)CSCCNC1=O]
compound DOTA-Ttds-Asp-Trp-5Clw-Tap-Glu-leu-[Cys(3MeBn)-Aib-Leu-Ile-AET] (GIP-095) of the following formula 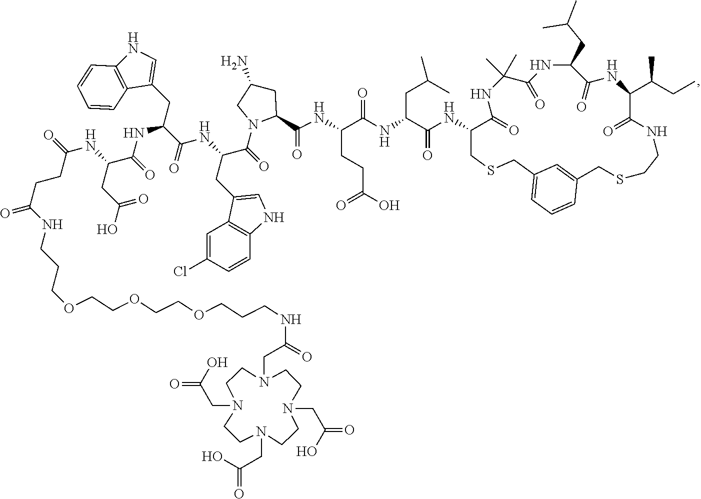 [SMILES: CC[C@H](C)[C@@H]1NC(=O)[C@H](CC(C)C)NC(=O)C(C)(C)NC(=O)[C@@H](NC(=O)[C@@H](CC(C)C)NC(=O)[C@H](CCC(=O)O)NC(=O)[C@@H]2C[C@@H](N)CN2C(=O)[C@H](Cc2c[nH]c3ccc(Cl)cc23)NC(=O)[C@H](Cc2c[nH]c3ccccc23)NC(=O)[C@H](CC(=O)O)NC(=O)CCC(=O)NCCCOCCOCCOCCCNC(=O)CN2CCN(CC(=O)O)CCN(CC(=O)O)CCN(CC(=O)O)CC2)CSCc2cccc(c2)CSCCNC1=O]
compound DOTA-Ttds-Asp-Trp-5Clw-4Ap-Glu-leu-[Cys(3MeBn)-Aib-Leu-Ile-AET] (GIP-096) of the following formula 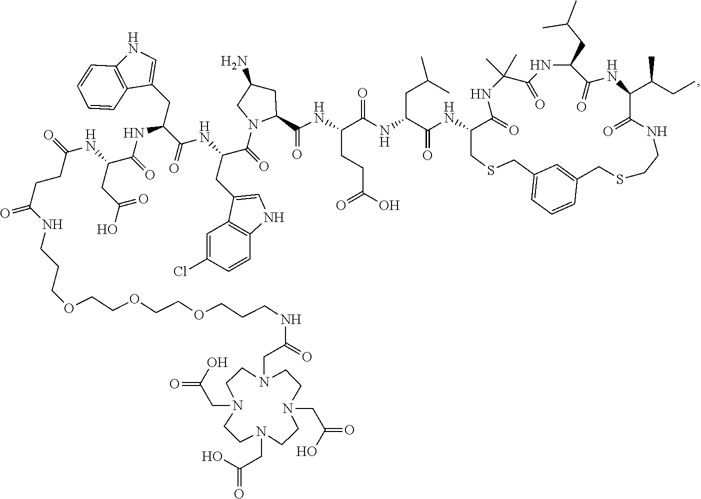 [SMILES: CC[C@H](C)[C@@H]1NC(=O)[C@H](CC(C)C)NC(=O)C(C)(C)NC(=O)[C@@H](NC(=O)[C@@H](CC(C)C)NC(=O)[C@H](CCC(=O)O)NC(=O)[C@@H]2C[C@H](N)CN2C(=O)[C@H](Cc2c[nH]c3ccc(Cl)cc23)NC(=O)[C@H](Cc2c[nH]c3ccccc23)NC(=O)[C@H](CC(=O)O)NC(=O)CCC(=O)NCCCOCCOCCOCCCNC(=O)CN2CCN(CC(=O)O)CCN(CC(=O)O)CCN(CC(=O)O)CC2)CSCc2cccc(c2)CSCCNC1=O]
compound DOTA-Ttds-Asp-Trp-5Clw-Pro-Glu-leu-[Cys(3MeBn)-Apc(Ac)-Leu-Ile-AET] (GIP-097) of the following formula 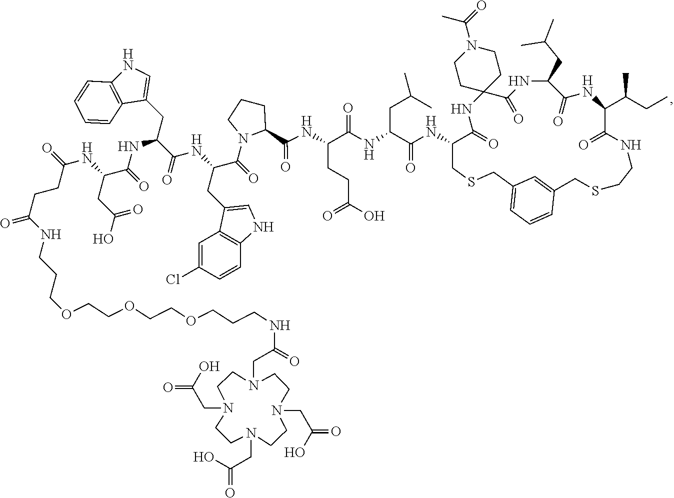 [SMILES: CC[C@H](C)[C@@H]1NC(=O)[C@H](CC(C)C)NC(=O)C2(CCN(C(C)=O)CC2)NC(=O)[C@@H](NC(=O)[C@@H](CC(C)C)NC(=O)[C@H](CCC(=O)O)NC(=O)[C@@H]2CCCN2C(=O)[C@H](Cc2c[nH]c3ccc(Cl)cc23)NC(=O)[C@H](Cc2c[nH]c3ccccc23)NC(=O)[C@H](CC(=O)O)NC(=O)CCC(=O)NCCCOCCOCCOCCCNC(=O)CN2CCN(CC(=O)O)CCN(CC(=O)O)CCN(CC(=O)O)CC2)CSCc2cccc(c2)CSCCNC1=O]
compound DOTA-Ttds-Asp-Trp-5Clw-Pro-Glu-leu-[Dap(3MeBn)-Aib-Leu-Ile-AETO] (GIP-098) of the following formula 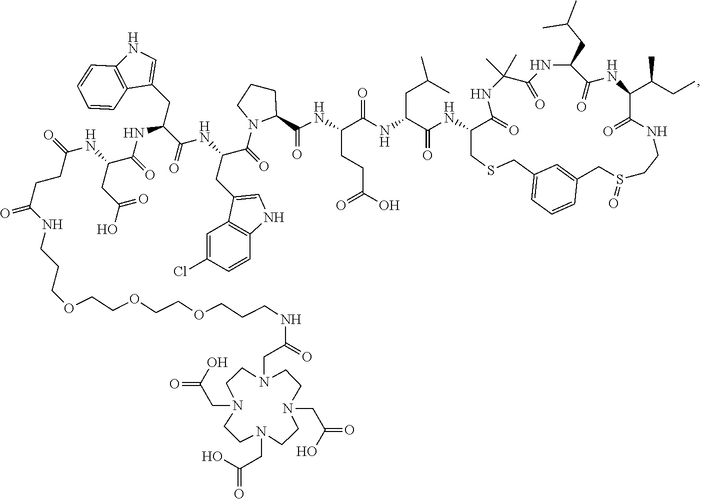 [SMILES: CC[C@H](C)[C@@H]1NC(=O)[C@H](CC(C)C)NC(=O)C(C)(C)NC(=O)[C@@H](NC(=O)[C@@H](CC(C)C)NC(=O)[C@H](CCC(=O)O)NC(=O)[C@@H]2CCCN2C(=O)[C@H](Cc2c[nH]c3ccc(Cl)cc23)NC(=O)[C@H](Cc2c[nH]c3ccccc23)NC(=O)[C@H](CC(=O)O)NC(=O)CCC(=O)NCCCOCCOCCOCCCNC(=O)CN2CCN(CC(=O)O)CCN(CC(=O)O)CCN(CC(=O)O)CC2)CSCc2cccc(c2)CS(=O)CCNC1=O]
compound DOTA-Ttds-Asp-Trp-5Clw-Pro-Glu-leu-[Cys(3MeBn)-Aib-Hle-Ile-AET] (GIP-099) of the following formula 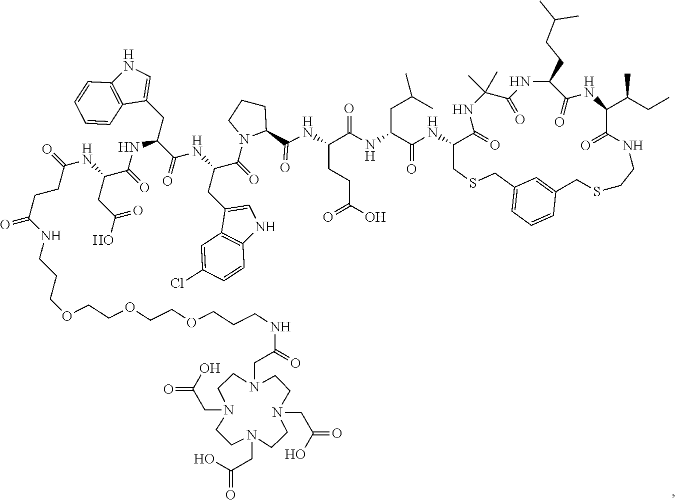 [SMILES: CC[C@H](C)[C@@H]1NC(=O)[C@H](CCC(C)C)NC(=O)C(C)(C)NC(=O)[C@@H](NC(=O)[C@@H](CC(C)C)NC(=O)[C@H](CCC(=O)O)NC(=O)[C@@H]2CCCN2C(=O)[C@H](Cc2c[nH]c3ccc(Cl)cc23)NC(=O)[C@H](Cc2c[nH]c3ccccc23)NC(=O)[C@H](CC(=O)O)NC(=O)CCC(=O)NCCCOCCOCCOCCCNC(=O)CN2CCN(CC(=O)O)CCN(CC(=O)O)CCN(CC(=O)O)CC2)CSCc2cccc(c2)CSCCNC1=O]
compound DOTA-Ttds-Asp-Trp-5Clw-Nmg-Glu-leu-[Cys(3MeBn)-Aib-Leu-Ile-AET] (GIP-100) of the following formula 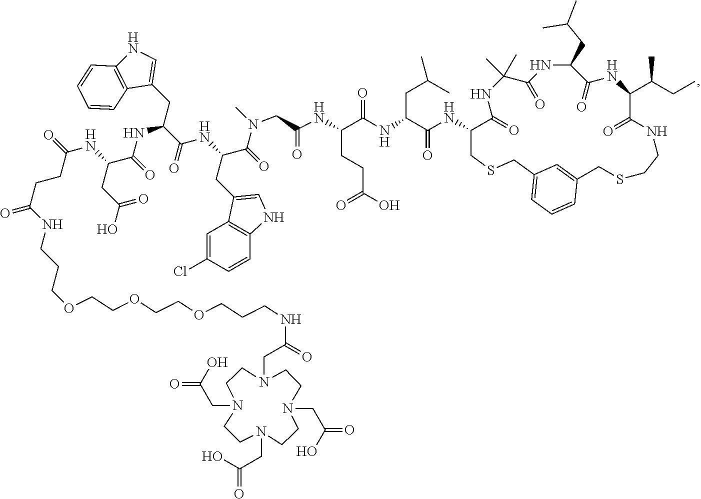
compound DOTA-Ttds-Asp-Trp-5Clw-Pro-Glu-leu-[Cys(3MeBn)-Ams-Leu-Ile-AET] (GIP-101) of the following formula 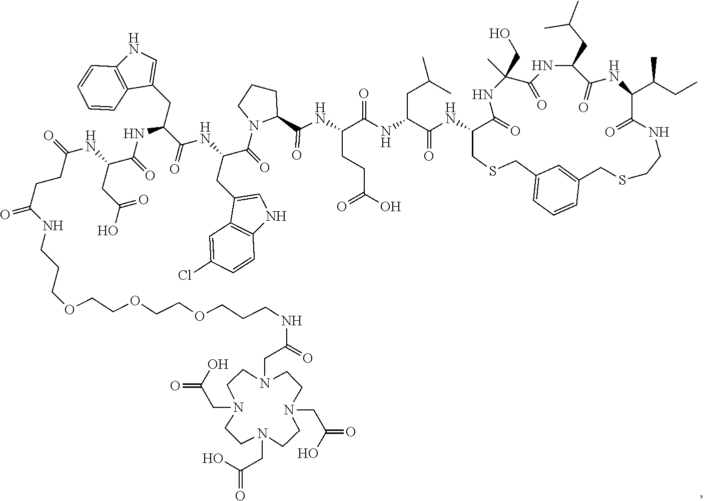 [SMILES: CC[C@H](C)[C@@H]1NC(=O)[C@H](CC(C)C)NC(=O)[C@](C)(CO)NC(=O)[C@@H](NC(=O)[C@@H](CC(C)C)NC(=O)[C@H](CCC(=O)O)NC(=O)[C@@H]2CCCN2C(=O)[C@H](Cc2c[nH]c3ccc(Cl)cc23)NC(=O)[C@H](Cc2c[nH]c3ccccc23)NC(=O)[C@H](CC(=O)O)NC(=O)CCC(=O)NCCCOCCOCCOCCCNC(=O)CN2CCN(CC(=O)O)CCN(CC(=O)O)CCN(CC(=O)O)CC2)CSCc2cccc(c2)CSCCNC1=O]
compound DOTA-Ttds-Asp-Trp-5Clw-H3p-Glu-leu-[Cys(3MeBn)-Aib-Leu-Ile-AET] (GIP-102) of the following formula 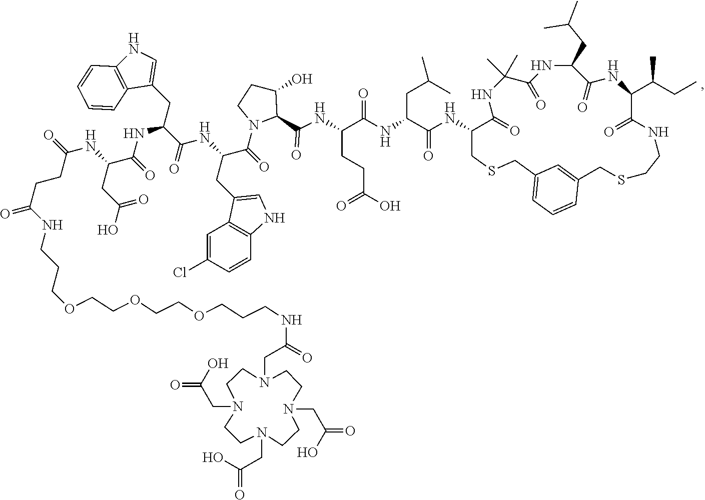 [SMILES: CC[C@H](C)[C@@H]1NC(=O)[C@H](CC(C)C)NC(=O)C(C)(C)NC(=O)[C@@H](NC(=O)[C@@H](CC(C)C)NC(=O)[C@H](CCC(=O)O)NC(=O)[C@@H]2[C@@H](O)CCN2C(=O)[C@H](Cc2c[nH]c3ccc(Cl)cc23)NC(=O)[C@H](Cc2c[nH]c3ccccc23)NC(=O)[C@H](CC(=O)O)NC(=O)CCC(=O)NCCCOCCOCCOCCCNC(=O)CN2CCN(CC(=O)O)CCN(CC(=O)O)CCN(CC(=O)O)CC2)CSCc2cccc(c2)CSCCNC1=O]
compound DOTA-APAc-Asp-Trp-5Clw-Hyp-Glu-leu-[Cys(2Lut)-Thp-Leu-Ile-AET] (GIP-103) of the following formula 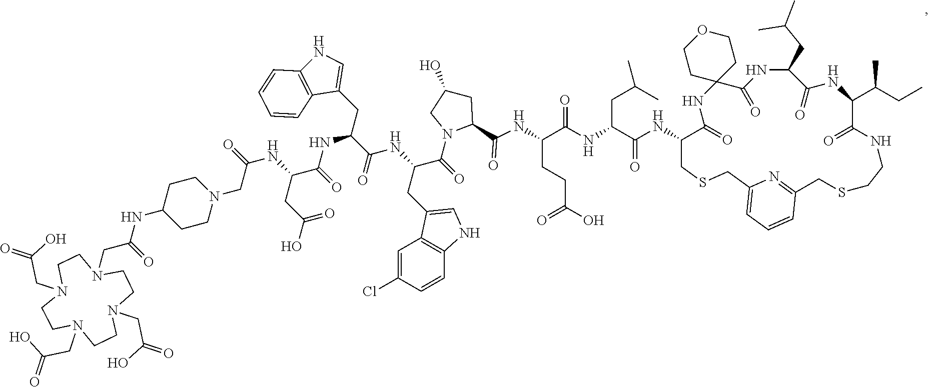 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CC(=O)O)NC(=O)CN2CCC(NC(=O)CN3CCN(CC(=O)O)CCN(CC(=O)O)CCN(CC(=O)O)CC3)CC2)CSCc2cccc(n2)CSCCNC1=O]
compound DOTA-Ttds-Asp-6Fw-5Clw-Pro-Glu-leu-[Cys(3MeBn)-Aib-Leu-Ile-AET] (GIP-104) of the following formula 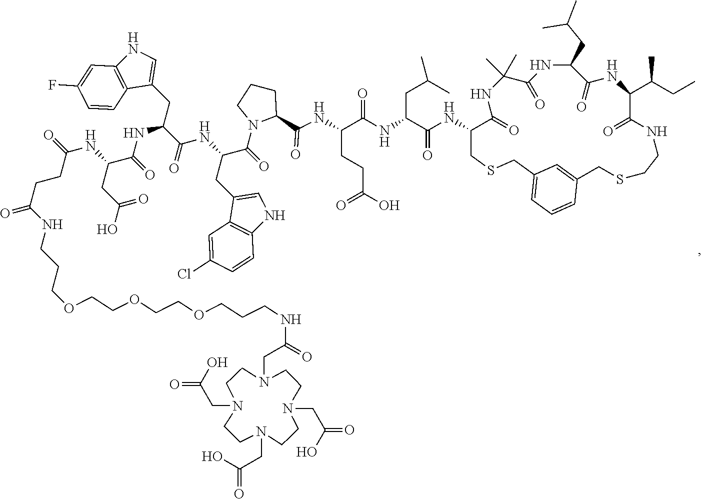 [SMILES: CC[C@H](C)[C@@H]1NC(=O)[C@H](CC(C)C)NC(=O)C(C)(C)NC(=O)[C@@H](NC(=O)[C@@H](CC(C)C)NC(=O)[C@H](CCC(=O)O)NC(=O)[C@@H]2CCCN2C(=O)[C@H](Cc2c[nH]c3ccc(Cl)cc23)NC(=O)[C@H](Cc2c[nH]c3cc(F)ccc23)NC(=O)[C@H](CC(=O)O)NC(=O)CCC(=O)NCCCOCCOCCOCCCNC(=O)CN2CCN(CC(=O)O)CCN(CC(=O)O)CCN(CC(=O)O)CC2)CSCc2cccc(c2)CSCCNC1=O]
compound DOTA-Ttds-Asp-6Fw-5Brw-Pro-Glu-leu-[Cys(3MeBn)-Aib-Leu-Ile-AET] (GIP-105) of the following formula 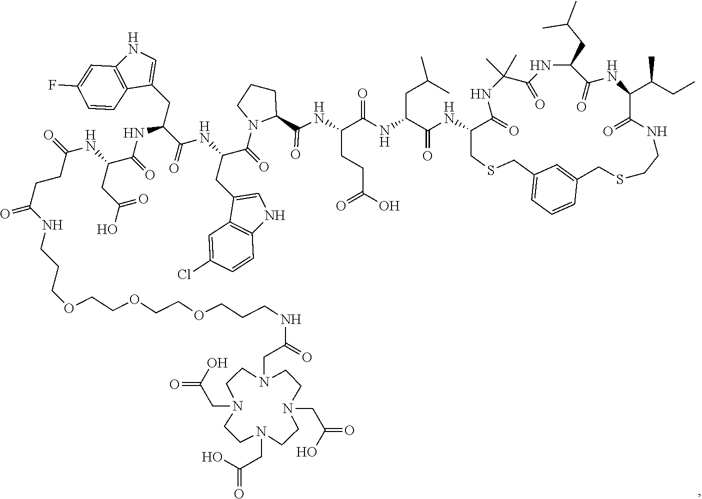 [SMILES: CC[C@H](C)[C@@H]1NC(=O)[C@H](CC(C)C)NC(=O)C(C)(C)NC(=O)[C@@H](NC(=O)[C@@H](CC(C)C)NC(=O)[C@H](CCC(=O)O)NC(=O)[C@@H]2CCCN2C(=O)[C@H](Cc2c[nH]c3ccc(Cl)cc23)NC(=O)[C@H](Cc2c[nH]c3cc(F)ccc23)NC(=O)[C@H](CC(=O)O)NC(=O)CCC(=O)NCCCOCCOCCOCCCNC(=O)CN2CCN(CC(=O)O)CCN(CC(=O)O)CCN(CC(=O)O)CC2)CSCc2cccc(c2)CSCCNC1=O]
compound DOTA-APAc-Asp-Trp-5Clw-Hyp-Glu-leu-[Dap(2Lut)-Thp-Leu-Ile-AET] (GIP-106) of the following formula 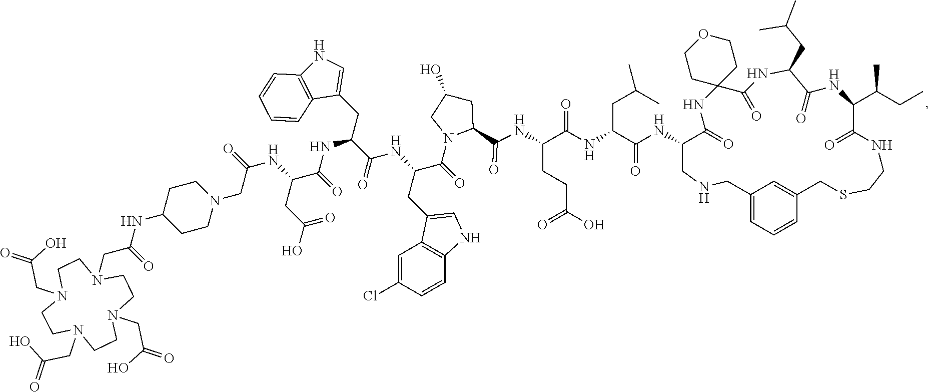 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CC(=O)O)NC(=O)CN2CCC(NC(=O)CN3CCN(CC(=O)O)CCN(CC(=O)O)CCN(CC(=O)O)CC3)CC2)CNCc2cccc(c2)CSCCNC1=O]
compound DOTA-APAc-Asp-Trp-5Clw-Hyp-Glu-leu-[Dap(3MeBn)-Thp-Leu-Ile-AET] (GIP-107) of the following formula 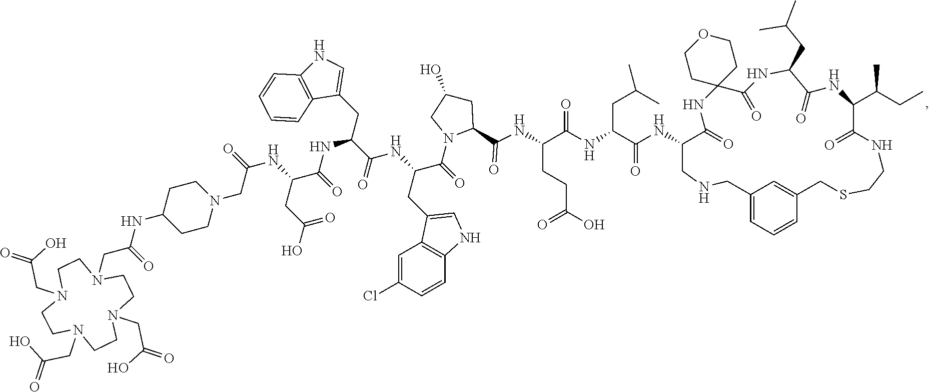 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CC(=O)O)NC(=O)CN2CCC(NC(=O)CN3CCN(CC(=O)O)CCN(CC(=O)O)CCN(CC(=O)O)CC3)CC2)CNCc2cccc(c2)CSCCNC1=O]
compound HO-Succinyl-Trp-5Clw-Hyp-Glu-leu-[Cys(2Lut)-Thp-Leu-Ile-Cys]-Ttds-Lys(DOTA)-NH₂ (GIP-108) of the following formula 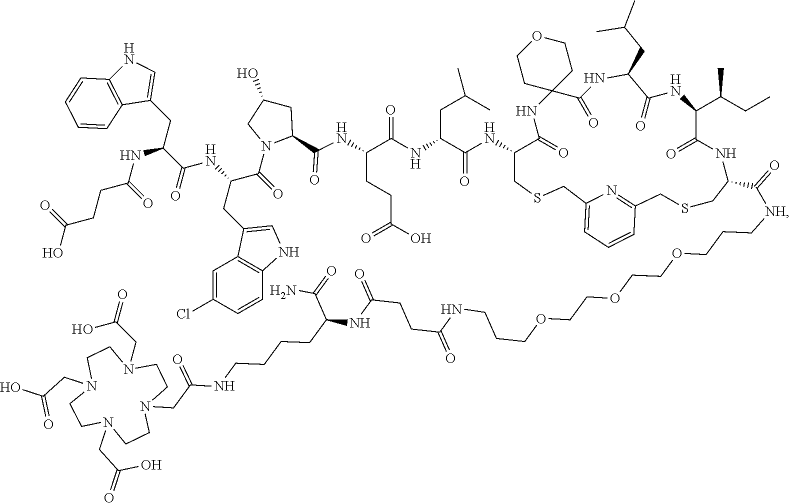 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)CCC(=O)O)CSCc2cccc(n2)CSC[C@@H](C(=O)NCCCOCCOCCOCCCNC(=O)CCC(=O)N[C@@H](CCCCNC(=O)CN2CCN(CC(=O)O)CCN(CC(=O)O)CCN(CC(=O)O)CC2)C(N)=O)NC1=O]
compound HO-Succinyl-Trp-5Clw-Hyp-Glu-leu-[Cys(2Lut)-Thp-Leu-Ile-Cys]-APAc-Lys(DOTA)-NH₂ (GIP-109) of the following formula 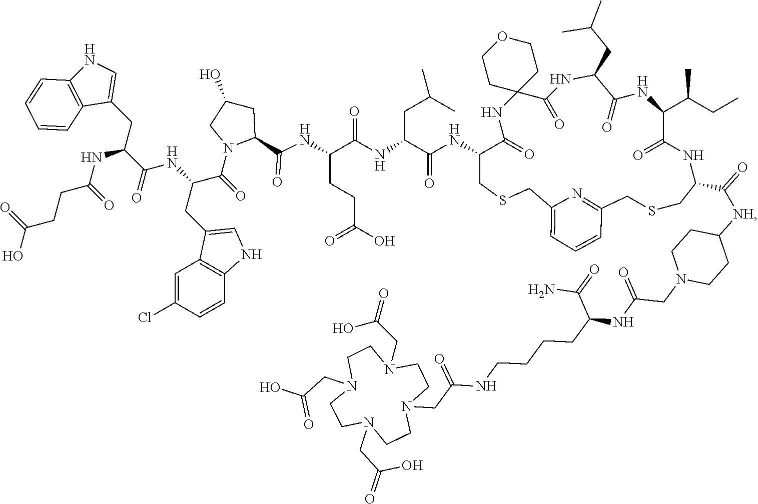 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)CCC(=O)O)CSCc2cccc(n2)CSC[C@@H](C(=O)NC2CCN(CC(=O)N[C@@H](CCCCNC(=O)CN3CCN(CC(=O)O)CCN(CC(=O)O)CCN(CC(=O)O)CC3)C(N)=O)CC2)NC1=O]
compound HO-Succinyl-Trp-5Clw-Hyp-Glu-leu-[Cys(2Lut)-Apc(DOTA)-Leu-Ile-AET] (GIP-110) of the following formula 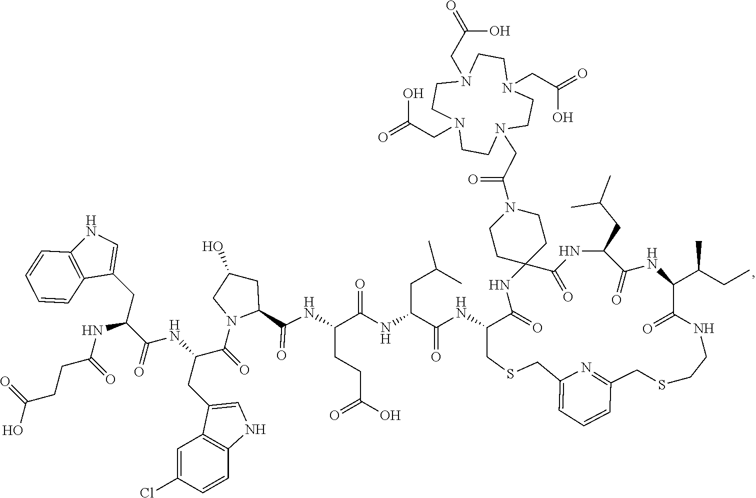 [SMILES: CC[C@H](C)[C@@H]1NC(=O)[C@H](CC(C)C)NC(=O)C2(CCN(C(=O)CN3CCN(CC(=O)O)CCN(CC(=O)O)CCN(CC(=O)O)CC3)CC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)CCC(=O)O)CSCc2cccc(n2)CSCCNC1=O]
compound DOTA-Ttds-Asp-Trp-5Clw-4Ap(Ac)-Glu-leu-[Cys(3MeBn)-Aib-Leu-Ile-AET] (GIP-111) of the following formula 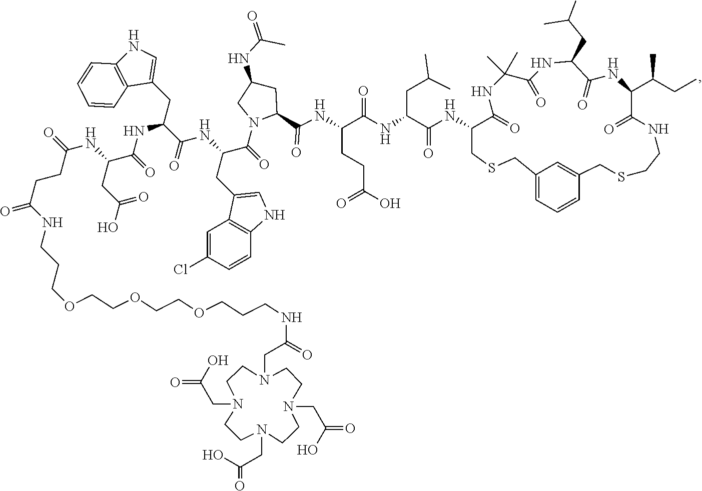 [SMILES: CC[C@H](C)[C@@H]1NC(=O)[C@H](CC(C)C)NC(=O)C(C)(C)NC(=O)[C@@H](NC(=O)[C@@H](CC(C)C)NC(=O)[C@H](CCC(=O)O)NC(=O)[C@@H]2C[C@H](NC(C)=O)CN2C(=O)[C@H](Cc2c[nH]c3ccc(Cl)cc23)NC(=O)[C@H](Cc2c[nH]c3ccccc23)NC(=O)[C@H](CC(=O)O)NC(=O)CCC(=O)NCCCOCCOCCOCCCNC(=O)CN2CCN(CC(=O)O)CCN(CC(=O)O)CCN(CC(=O)O)CC2)CSCc2cccc(c2)CSCCNC1=O]
compound DOTA-Ttds-Asp-Trp-5Clw-Tap-Glu-leu-[Cys(2Lut)-Apc-Leu-Ile-AET] (GIP-112) of the following formula 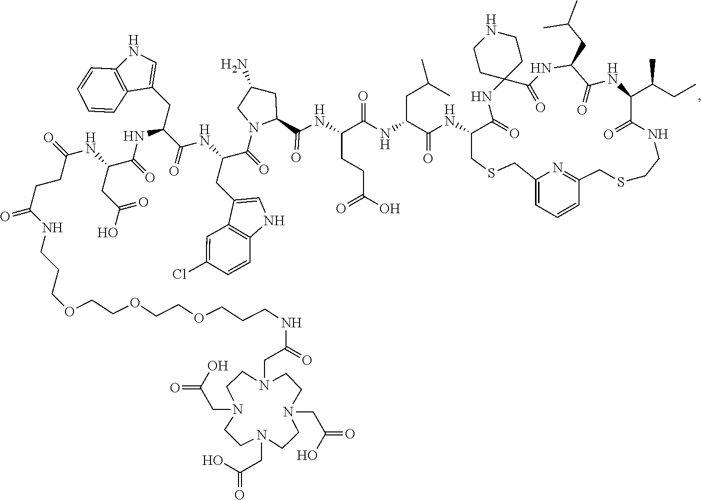 [SMILES: CC[C@H](C)[C@@H]1NC(=O)[C@H](CC(C)C)NC(=O)C2(CCNCC2)NC(=O)[C@@H](NC(=O)[C@@H](CC(C)C)NC(=O)[C@H](CCC(=O)O)NC(=O)[C@@H]2C[C@@H](N)CN2C(=O)[C@H](Cc2c[nH]c3ccc(Cl)cc23)NC(=O)[C@H](Cc2c[nH]c3ccccc23)NC(=O)[C@H](CC(=O)O)NC(=O)CCC(=O)NCCCOCCOCCOCCCNC(=O)CN2CCN(CC(=O)O)CCN(CC(=O)O)CCN(CC(=O)O)CC2)CSCc2cccc(n2)CSCCNC1=O]
compound DOTA-Ttds-Asp-Trp-5Clw-Tap-Glu-leu-[Dap(2Lut)-Apc-Leu-Ile-AET] (GIP-113) of the following formula 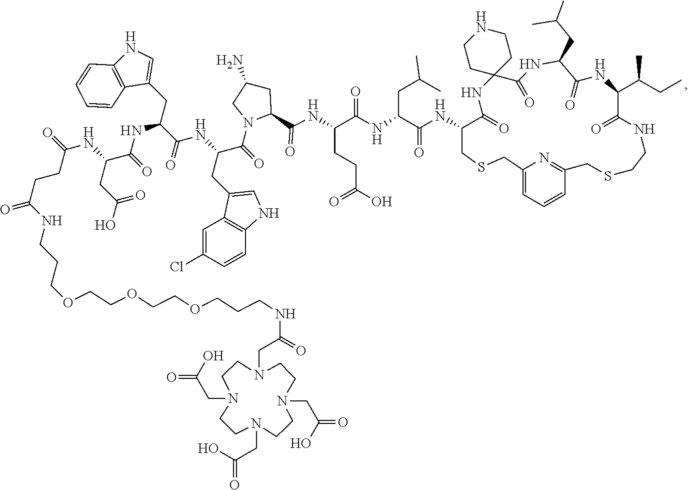 [SMILES: CC[C@H](C)[C@@H]1NC(=O)[C@H](CC(C)C)NC(=O)C2(CCNCC2)NC(=O)[C@@H](NC(=O)[C@@H](CC(C)C)NC(=O)[C@H](CCC(=O)O)NC(=O)[C@@H]2C[C@@H](N)CN2C(=O)[C@H](Cc2c[nH]c3ccc(Cl)cc23)NC(=O)[C@H](Cc2c[nH]c3ccccc23)NC(=O)[C@H](CC(=O)O)NC(=O)CCC(=O)NCCCOCCOCCOCCCNC(=O)CN2CCN(CC(=O)O)CCN(CC(=O)O)CCN(CC(=O)O)CC2)CSCc2cccc(n2)CSCCNC1=O]
compound HO-Succinyl-Trp-5Clw-Hyp-Glu-leu-[Cys(2Lut)-Apc-Leu-Ile-Cys]-APAc-Lys(DOTA)-NH₂ (GIP-114) of the following formula 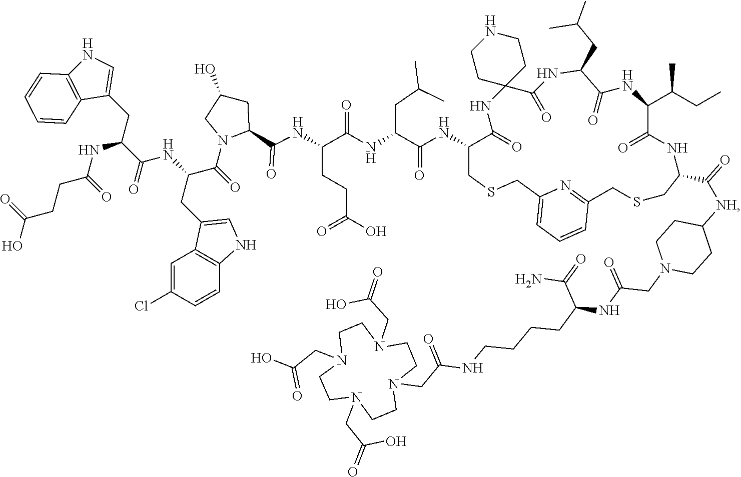 [SMILES: CC[C@H](C)[C@@H]1NC(=O)[C@H](CC(C)C)NC(=O)C2(CCNCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)CCC(=O)O)CSCc2cccc(n2)CSC[C@@H](C(=O)NC2CCN(CC(=O)N[C@@H](CCCCNC(=O)CN3CCN(CC(=O)O)CCN(CC(=O)O)CCN(CC(=O)O)CC3)C(N)=O)CC2)NC1=O]
compound HO-Succinyl-Trp-5Clw-Hyp-Glu-leu-[Cys(2Lut)-Thp-Leu-Ile-Cys]-Lys(DOTA)-NH₂ (GIP-115) of the following formula 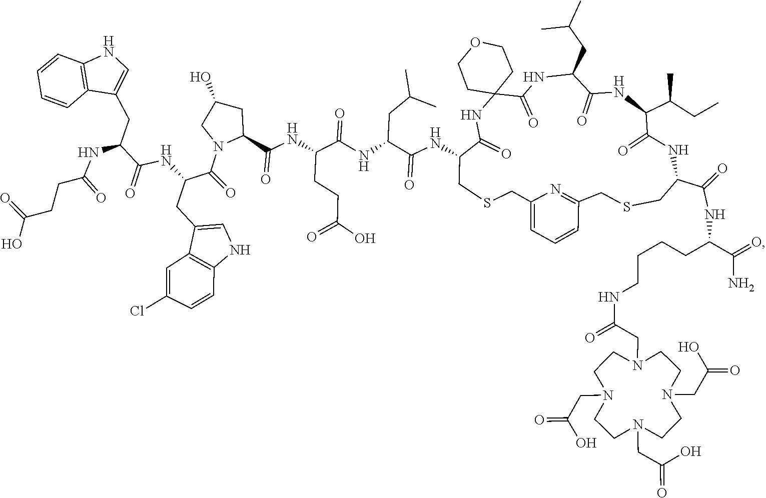 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)CCC(=O)O)CSCc2cccc(n2)CSC[C@@H](C(=O)N[C@@H](CCCCNC(=O)CN2CCN(CC(=O)O)CCN(CC(=O)O)CCN(CC(=O)O)CC2)C(N)=O)NC1=O]
compound HO-Succinyl-Trp-5Clw-Hyp-Glu-leu-[Cys(2Lut)-Thp-Leu-Ile-Cys]-lys(DOTA)-NH₂ (GIP-116) of the following formula 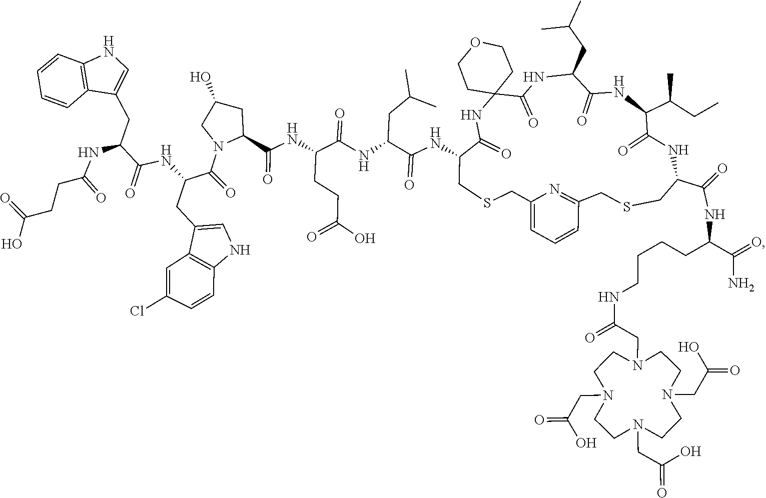 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)CCC(=O)O)CSCc2cccc(n2)CSC[C@@H](C(=O)N[C@H](CCCCNC(=O)CN2CCN(CC(=O)O)CCN(CC(=O)O)CCN(CC(=O)O)CC2)C(N)=O)NC1=O]
compound HO-Succinyl-Trp-5Clw-Hyp-Glu-leu-[Cys(2Lut)-Thp-Leu-Ile-Cys]-Ape-DOTA (GIP-117) of the following formula 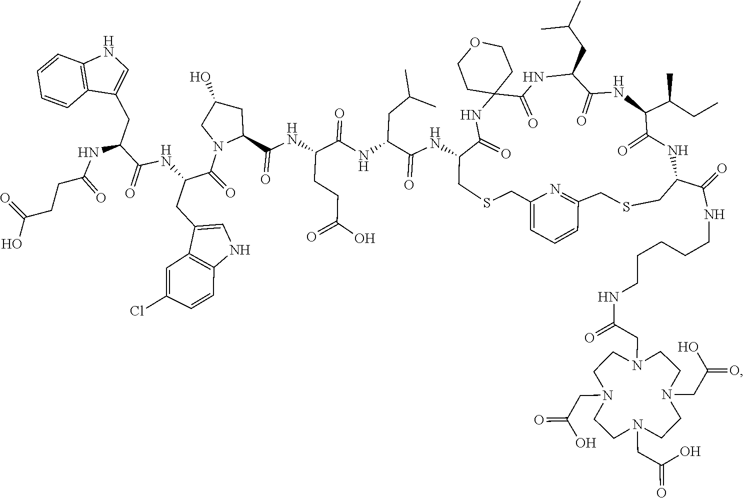 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)CCC(=O)O)CSCc2cccc(n2)CSC[C@@H](C(=O)NCCCCCNC(=O)CN2CCN(CC(=O)O)CCN(CC(=O)O)CCN(CC(=O)O)CC2)NC1=O]
compound HO-Succinyl-Trp-5Clw-Hyp-Glu-leu-[Cys(2Lut)-Thp-Leu-Ile-Cys]-PP-DOTA (GIP-118) of the following formula 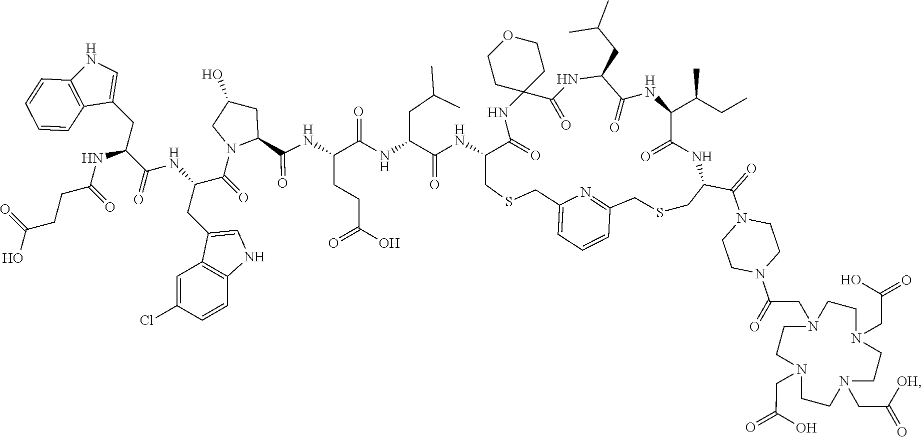 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)CCC(=O)O)CSCc2cccc(n2)CSC[C@@H](C(=O)N2CCN(C(=O)CN3CCN(CC(=O)O)CCN(CC(=O)O)CCN(CC(=O)O)CC3)CC2)NC1=O]
compound DOTA-Ttds-Asp-Trp-5Clw-Pro-Glu-leu-[Cys(2Lut)-Apc-Leu-Ile-AET] (GIP-119) of the following formula 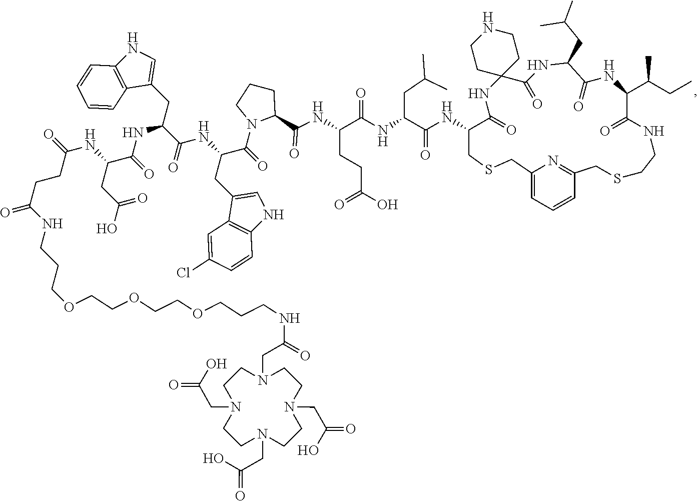 [SMILES: CC[C@H](C)[C@@H]1NC(=O)[C@H](CC(C)C)NC(=O)C2(CCNCC2)NC(=O)[C@@H](NC(=O)[C@@H](CC(C)C)NC(=O)[C@H](CCC(=O)O)NC(=O)[C@@H]2CCCN2C(=O)[C@H](Cc2c[nH]c3ccc(Cl)cc23)NC(=O)[C@H](Cc2c[nH]c3ccccc23)NC(=O)[C@H](CC(=O)O)NC(=O)CCC(=O)NCCCOCCOCCOCCCNC(=O)CN2CCN(CC(=O)O)CCN(CC(=O)O)CCN(CC(=O)O)CC2)CSCc2cccc(n2)CSCCNC1=O]
compound MeO2kDaPEGAc-Asp-Trp-5Clw-Hyp-Glu-leu-[Cys(2Lut)-Thp-Leu-Ile-Cys]-lys(DOTA)-NH₂ (GIP-120) of the following formula 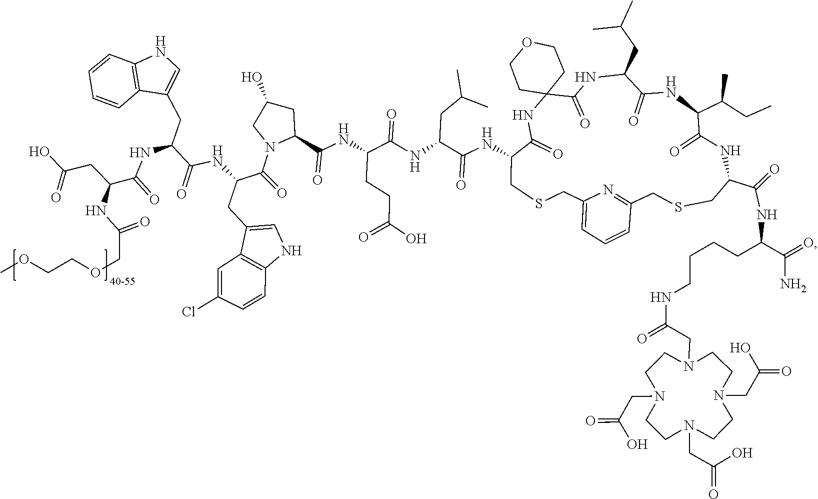
compound Ac-Asp-Trp-5Clw-Tap(DOTA)-Glu-leu-[Cys(2Lut)-Thp-Leu-Ile-Cys]-NH₂ (GIP-121) of the following formula 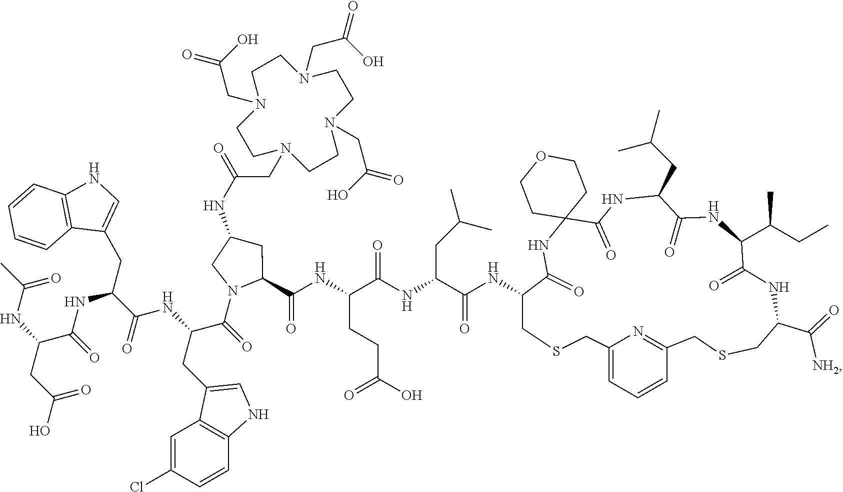 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](NC(=O)CN3CCN(CC(=O)O)CCN(CC(=O)O)CCN(CC(=O)O)CC3)CN2C(=O)[C@H](Cc2c[nH]c3ccc(Cl)cc23)NC(=O)[C@H](Cc2c[nH]c3ccccc23)NC(=O)[C@H](CC(=O)O)NC(C)=O)CSCc2cccc(n2)CSC[C@@H](C(N)=O)NC1=O]
compound Ac-Asp-Trp-5Clw-Hyp-Glu-leu-[Cys(2Lut)-Apc(DOTA)-Leu-Ile-Cys]-NH₂ (GIP-122) of the following formula 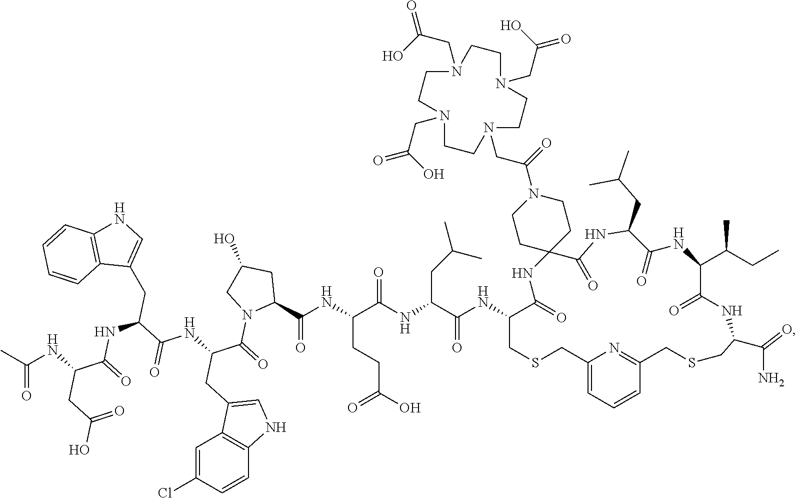 [SMILES: CC[C@H](C)[C@@H]1NC(=O)[C@H](CC(C)C)NC(=O)C2(CCN(C(=O)CN3CCN(CC(=O)O)CCN(CC(=O)O)CCN(CC(=O)O)CC3)CC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CC(=O)O)NC(C)=O)CSCc2cccc(n2)CSC[C@@H](C(N)=O)NC1=O]
compound Ac-Asp-Trp-5Clw-Tap-Glu-leu-[Cys(2Lut)-Apc(DOTA)-Leu-Ile-Cys]-NH₂ (GIP-123) of the following formula 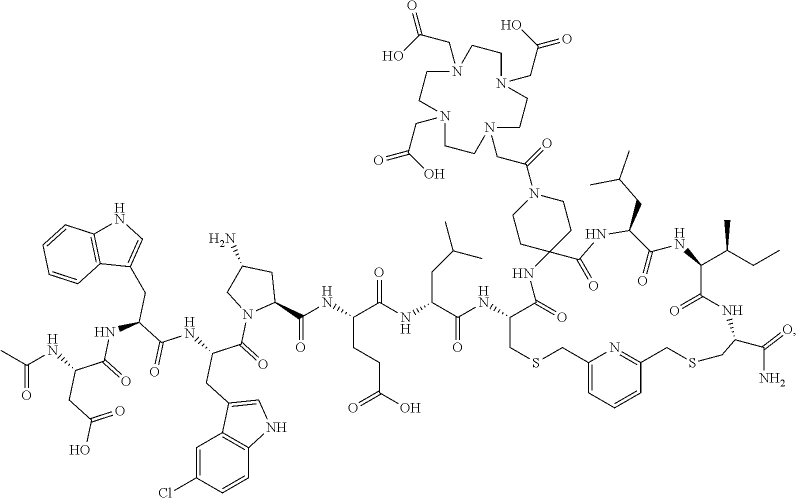 [SMILES: CC[C@H](C)[C@@H]1NC(=O)[C@H](CC(C)C)NC(=O)C2(CCN(C(=O)CN3CCN(CC(=O)O)CCN(CC(=O)O)CCN(CC(=O)O)CC3)CC2)NC(=O)[C@@H](NC(=O)[C@@H](CC(C)C)NC(=O)[C@H](CCC(=O)O)NC(=O)[C@@H]2C[C@@H](N)CN2C(=O)[C@H](Cc2c[nH]c3ccc(Cl)cc23)NC(=O)[C@H](Cc2c[nH]c3ccccc23)NC(=O)[C@H](CC(=O)O)NC(C)=O)CSCc2cccc(n2)CSC[C@@H](C(N)=O)NC1=O]
compound Ac-Asp-Trp-5Clw-Hyp-Glu-leu-[Cys(tMeBn(DOTA-AET))-Thp-Leu-Ile-Cys]-NH₂ (GIP-124) of the following formula 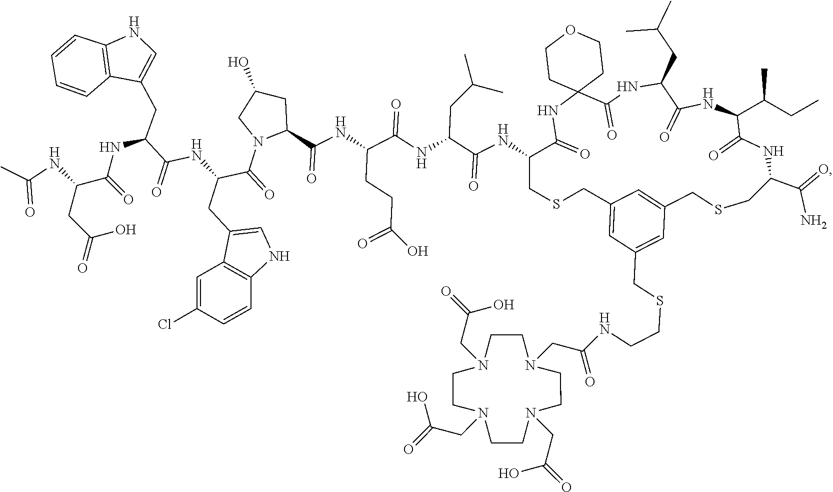 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CC(=O)O)NC(C)=O)CSCc2cc(CSCCNC(=O)CN3CCN(CC(=O)O)CCN(CC(=O)O)CCN(CC(=O)O)CC3)cc(c2)CSC[C@@H](C(N)=O)NC1=O]
compound DOTA-Asp-Trp-5Clw-Hyp-Glu-leu-[Cys(2Lut)-Thp-Leu-Ile-Cys]-NH₂ (GIP-125) of the following formula 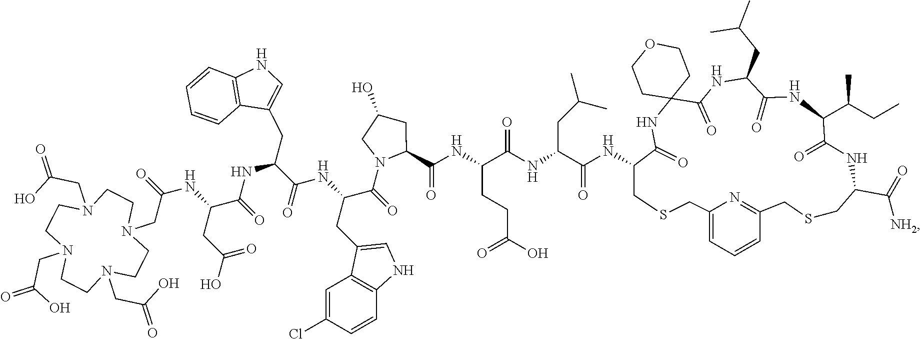 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CC(=O)O)NC(=O)CN2CCN(CC(=O)O)CCN(CC(=O)O)CCN(CC(=O)O)CC2)CSCc2cccc(n2)CSC[C@@H](C(N)=O)NC1=O]
compound DOTA-Ttds-Tyr-Asp-Trp-Trp-Pro-Glu-Leu-[Cys(3MeBn)-Ala-Leu-Ile-Cys]-NH₂ (GIP-126) of the following formula 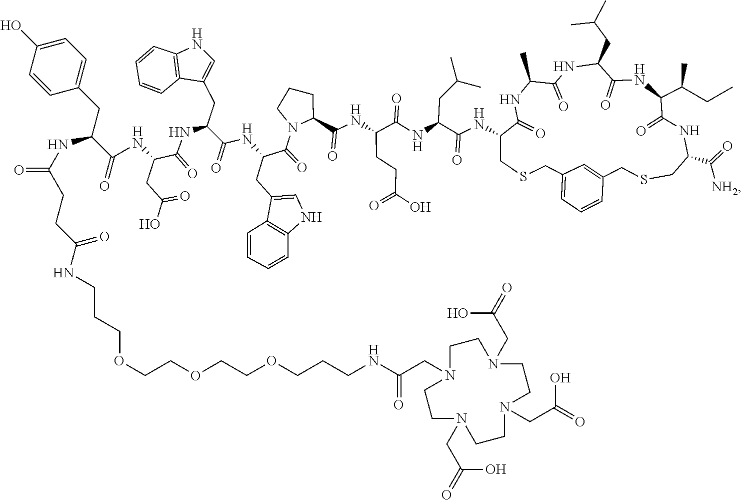 [SMILES: CC[C@H](C)[C@@H]1NC(=O)[C@H](CC(C)C)NC(=O)[C@H](C)NC(=O)[C@@H](NC(=O)[C@H](CC(C)C)NC(=O)[C@H](CCC(=O)O)NC(=O)[C@@H]2CCCN2C(=O)[C@H](Cc2c[nH]c3ccccc23)NC(=O)[C@H](Cc2c[nH]c3ccccc23)NC(=O)[C@H](CC(=O)O)NC(=O)[C@H](Cc2ccc(O)cc2)NC(=O)CCC(=O)NCCCOCCOCCOCCCNC(=O)CN2CCN(CC(=O)O)CCN(CC(=O)O)CCN(CC(=O)O)CC2)CSCc2cccc(c2)CSC[C@@H](C(N)=O)NC1=O]
compound Ac-Tyr-Asp-Trp-Trp-Pro-Glu-Leu-[Cys(3MeBn)-Ala-Leu-Ile-Cys]-Ttds-Lys(DOTA)-NH₂ (GIP-127) of the following formula 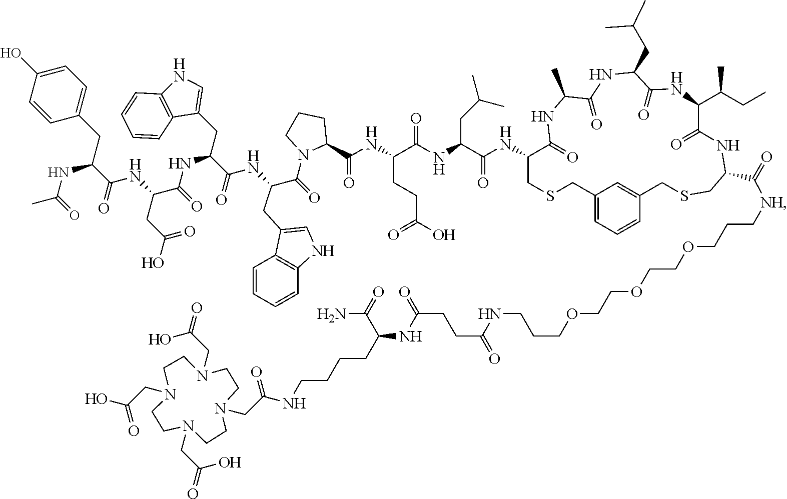 [SMILES: CC[C@H](C)[C@@H]1NC(=O)[C@H](CC(C)C)NC(=O)[C@H](C)NC(=O)[C@@H](NC(=O)[C@H](CC(C)C)NC(=O)[C@H](CCC(=O)O)NC(=O)[C@@H]2CCCN2C(=O)[C@H](Cc2c[nH]c3ccccc23)NC(=O)[C@H](Cc2c[nH]c3ccccc23)NC(=O)[C@H](CC(=O)O)NC(=O)[C@H](Cc2ccc(O)cc2)NC(C)=O)CSCc2cccc(c2)CSC[C@@H](C(=O)NCCCOCCOCCOCCCNC(=O)CCC(=O)N[C@@H](CCCCNC(=O)CN2CCN(CC(=O)O)CCN(CC(=O)O)CCN(CC(=O)O)CC2)C(N)=O)NC1=O]
compound DOTA-Ttds-Asp-Trp-Trp-Pro-Glu-leu-[Cys(3MeBn)-Aib-Leu-Ile-Cys]-NH₂ (GIP-128) of the following formula 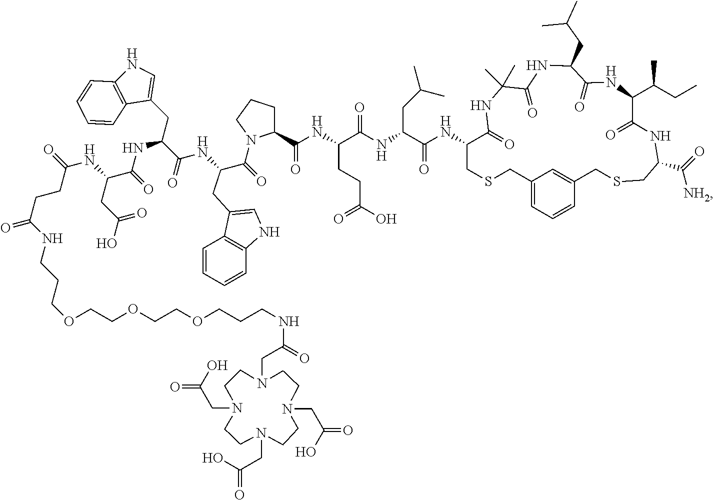
compound DOTA-Ttds-Asp-Trp-Trp-Pro-Glu-leu-[Cys(3MeBn)-Aib-Leu-Ile-AET] (GIP-129) of the following formula 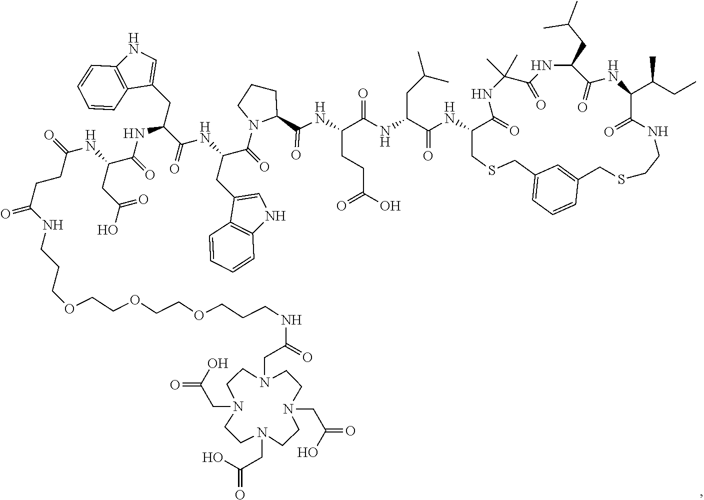 [SMILES: CC[C@H](C)[C@@H]1NC(=O)[C@H](CC(C)C)NC(=O)C(C)(C)NC(=O)[C@@H](NC(=O)[C@@H](CC(C)C)NC(=O)[C@H](CCC(=O)O)NC(=O)[C@@H]2CCCN2C(=O)[C@H](Cc2c[nH]c3ccccc23)NC(=O)[C@H](Cc2c[nH]c3ccccc23)NC(=O)[C@H](CC(=O)O)NC(=O)CCC(=O)NCCCOCCOCCOCCCNC(=O)CN2CCN(CC(=O)O)CCN(CC(=O)O)CCN(CC(=O)O)CC2)CSCc2cccc(c2)CSCCNC1=O]
compound DOTA-Ttds-Nmd-Trp-Trp-Pro-Glu-Leu-[Cys(3MeBn)-Aib-Leu-Ile-AET] (GIP-130) of the following formula 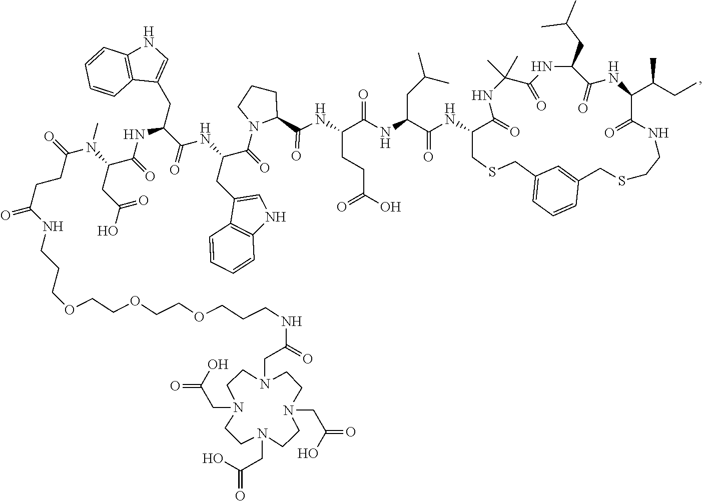 [SMILES: CC[C@H](C)[C@@H]1NC(=O)[C@H](CC(C)C)NC(=O)C(C)(C)NC(=O)[C@@H](NC(=O)[C@H](CC(C)C)NC(=O)[C@H](CCC(=O)O)NC(=O)[C@@H]2CCCN2C(=O)[C@H](Cc2c[nH]c3ccccc23)NC(=O)[C@H](Cc2c[nH]c3ccccc23)NC(=O)[C@H](CC(=O)O)N(C)C(=O)CCC(=O)NCCCOCCOCCOCCCNC(=O)CN2CCN(CC(=O)O)CCN(CC(=O)O)CCN(CC(=O)O)CC2)CSCc2cccc(c2)CSCCNC1=O]
compound Ac-Asp-Trp-Trp-Pro-Glu-Leu-[Cys(3MeBn)-Aib-Leu-Ile-Cys]-Ttds-Lys(DOTA)-NH₂ (GIP-131) of the following formula 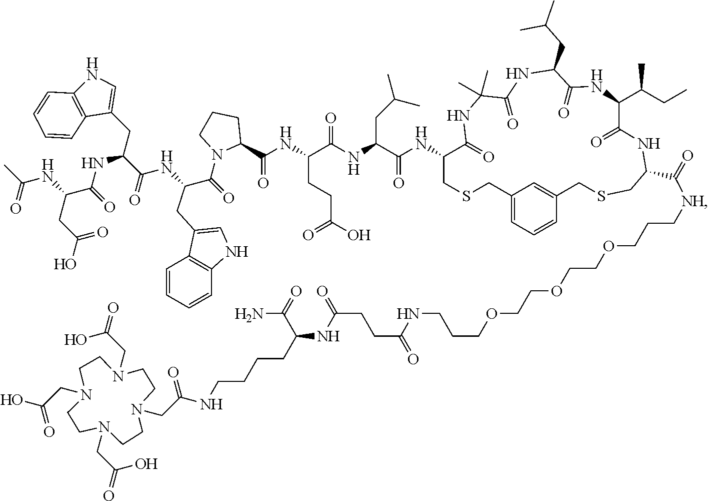 [SMILES: CC[C@H](C)[C@@H]1NC(=O)[C@H](CC(C)C)NC(=O)C(C)(C)NC(=O)[C@@H](NC(=O)[C@H](CC(C)C)NC(=O)[C@H](CCC(=O)O)NC(=O)[C@@H]2CCCN2C(=O)[C@H](Cc2c[nH]c3ccccc23)NC(=O)[C@H](Cc2c[nH]c3ccccc23)NC(=O)[C@H](CC(=O)O)NC(C)=O)CSCc2cccc(c2)CSC[C@@H](C(=O)NCCCOCCOCCOCCCNC(=O)CCC(=O)N[C@@H](CCCCNC(=O)CN2CCN(CC(=O)O)CCN(CC(=O)O)CCN(CC(=O)O)CC2)C(N)=O)NC1=O]
compound Ac-Asp-Trp-Trp-Pro-Glu-Npg-[Cys(3MeBn)-Aib-Leu-Ile-Cys]-Ttds-Lys(DOTA)-NH₂ (GIP-132) of the following formula 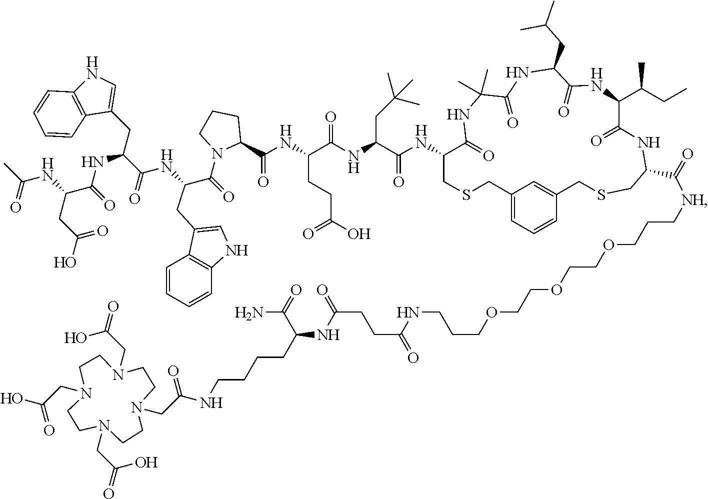 [SMILES: CC[C@H](C)[C@@H]1NC(=O)[C@H](CC(C)C)NC(=O)C(C)(C)NC(=O)[C@@H](NC(=O)[C@H](CC(C)(C)C)NC(=O)[C@H](CCC(=O)O)NC(=O)[C@@H]2CCCN2C(=O)[C@H](Cc2c[nH]c3ccccc23)NC(=O)[C@H](Cc2c[nH]c3ccccc23)NC(=O)[C@H](CC(=O)O)NC(C)=O)CSCc2cccc(c2)CSC[C@@H](C(=O)NCCCOCCOCCOCCCNC(=O)CCC(=O)N[C@@H](CCCCNC(=O)CN2CCN(CC(=O)O)CCN(CC(=O)O)CCN(CC(=O)O)CC2)C(N)=O)NC1=O]
compound Ac-Asp-Trp-Trp-Pro-Glu-Leu-[Cys(3MeBn)-Aib-Npg-Ile-Cys]-Ttds-Lys(DOTA)-NH₂ (GIP-133) of the following formula 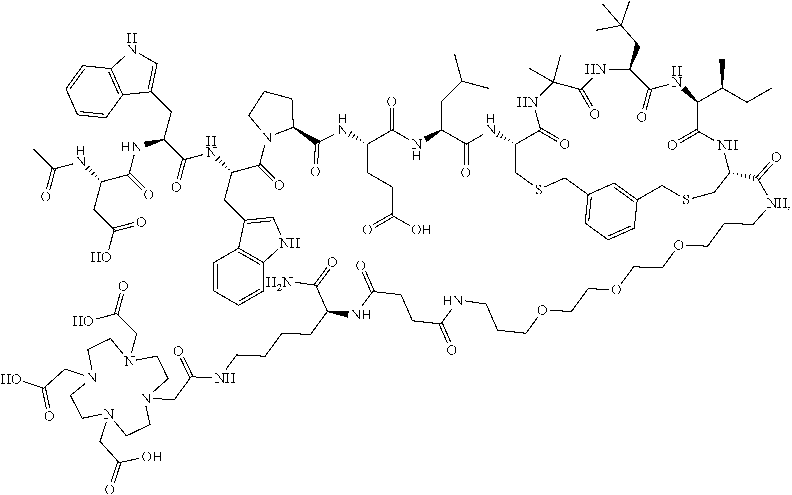 [SMILES: CC[C@H](C)[C@@H]1NC(=O)[C@H](CC(C)(C)C)NC(=O)C(C)(C)NC(=O)[C@@H](NC(=O)[C@H](CC(C)C)NC(=O)[C@H](CCC(=O)O)NC(=O)[C@@H]2CCCN2C(=O)[C@H](Cc2c[nH]c3ccccc23)NC(=O)[C@H](Cc2c[nH]c3ccccc23)NC(=O)[C@H](CC(=O)O)NC(C)=O)CSCc2cccc(c2)CSC[C@@H](C(=O)NCCCOCCOCCOCCCNC(=O)CCC(=O)N[C@@H](CCCCNC(=O)CN2CCN(CC(=O)O)CCN(CC(=O)O)CCN(CC(=O)O)CC2)C(N)=O)NC1=O]
compound Ac-Asp-Trp-Trp-Pro-Glu-Leu-[Cys(3MeBn)-Aib-Cha-Ile-Cys]-Ttds-Lys(DOTA)-NH₂ (GIP-134) of the following formula 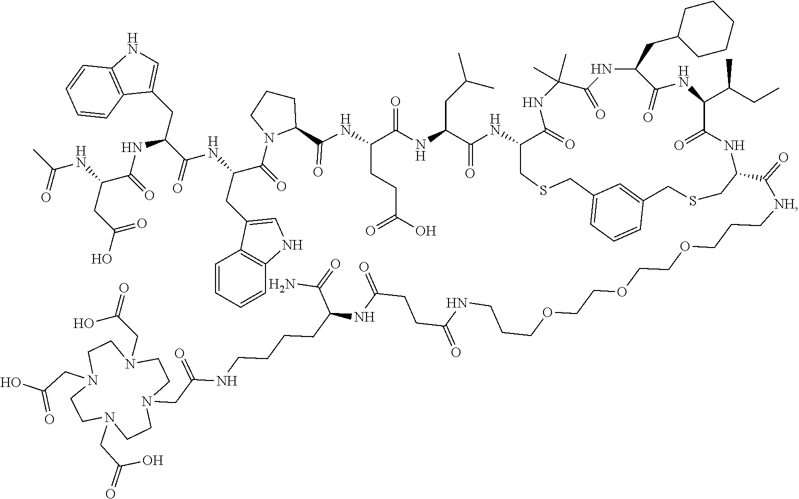 [SMILES: CC[C@H](C)[C@@H]1NC(=O)[C@H](CC2CCCCC2)NC(=O)C(C)(C)NC(=O)[C@@H](NC(=O)[C@H](CC(C)C)NC(=O)[C@H](CCC(=O)O)NC(=O)[C@@H]2CCCN2C(=O)[C@H](Cc2c[nH]c3ccccc23)NC(=O)[C@H](Cc2c[nH]c3ccccc23)NC(=O)[C@H](CC(=O)O)NC(C)=O)CSCc2cccc(c2)CSC[C@@H](C(=O)NCCCOCCOCCOCCCNC(=O)CCC(=O)N[C@@H](CCCCNC(=O)CN2CCN(CC(=O)O)CCN(CC(=O)O)CCN(CC(=O)O)CC2)C(N)=O)NC1=O]
compound Php-Asp-Trp-Trp-Pro-Glu-leu-[Cys(3MeBn)-Aib-Leu-Ile-Cys]-Ttds-Lys(DOTA)-NH₂ (GIP-135) of the following formula 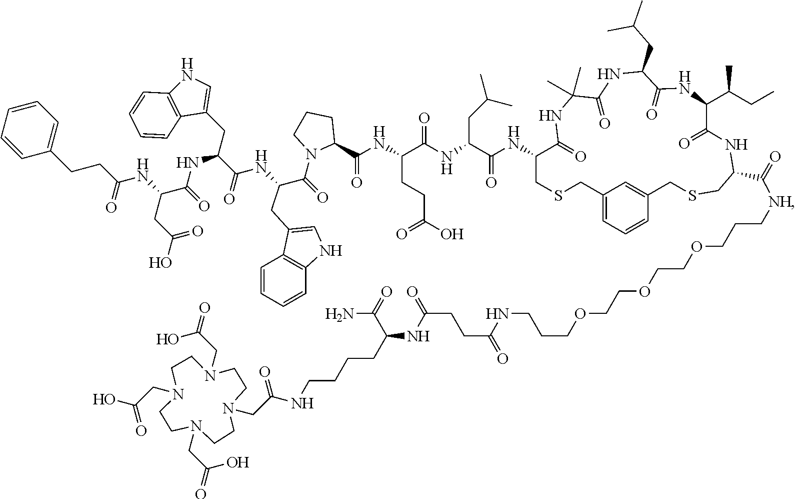 [SMILES: CC[C@H](C)[C@@H]1NC(=O)[C@H](CC(C)C)NC(=O)C(C)(C)NC(=O)[C@@H](NC(=O)[C@@H](CC(C)C)NC(=O)[C@H](CCC(=O)O)NC(=O)[C@@H]2CCCN2C(=O)[C@H](Cc2c[nH]c3ccccc23)NC(=O)[C@H](Cc2c[nH]c3ccccc23)NC(=O)[C@H](CC(=O)O)NC(=O)CCc2ccccc2)CSCc2cccc(c2)CSC[C@@H](C(=O)NCCCOCCOCCOCCCNC(=O)CCC(=O)N[C@@H](CCCCNC(=O)CN2CCN(CC(=O)O)CCN(CC(=O)O)CCN(CC(=O)O)CC2)C(N)=O)NC1=O]
compound 4FPhp-Asp-Trp-Trp-Pro-Glu-leu-[Cys(3MeBn)-Aib-Leu-Ile-Cys]-Ttds-Lys(DOTA)-NH₂ (GIP-136) of the following formula 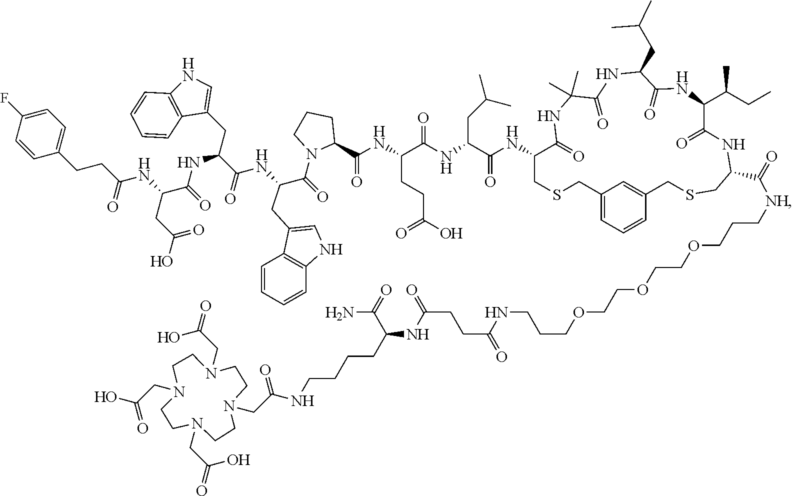 [SMILES: CC[C@H](C)[C@@H]1NC(=O)[C@H](CC(C)C)NC(=O)C(C)(C)NC(=O)[C@@H](NC(=O)[C@@H](CC(C)C)NC(=O)[C@H](CCC(=O)O)NC(=O)[C@@H]2CCCN2C(=O)[C@H](Cc2c[nH]c3ccccc23)NC(=O)[C@H](Cc2c[nH]c3ccccc23)NC(=O)[C@H](CC(=O)O)NC(=O)CCc2ccc(F)cc2)CSCc2cccc(c2)CSC[C@@H](C(=O)NCCCOCCOCCOCCCNC(=O)CCC(=O)N[C@@H](CCCCNC(=O)CN2CCN(CC(=O)O)CCN(CC(=O)O)CCN(CC(=O)O)CC2)C(N)=O)NC1=O]
compound 4OHPhp-Asp-Trp-Trp-Pro-Glu-leu-[Cys(3MeBn)-Aib-Leu-Ile-Cys]-Ttds-Lys(DOTA)-NH₂ (GIP-137) of the following formula 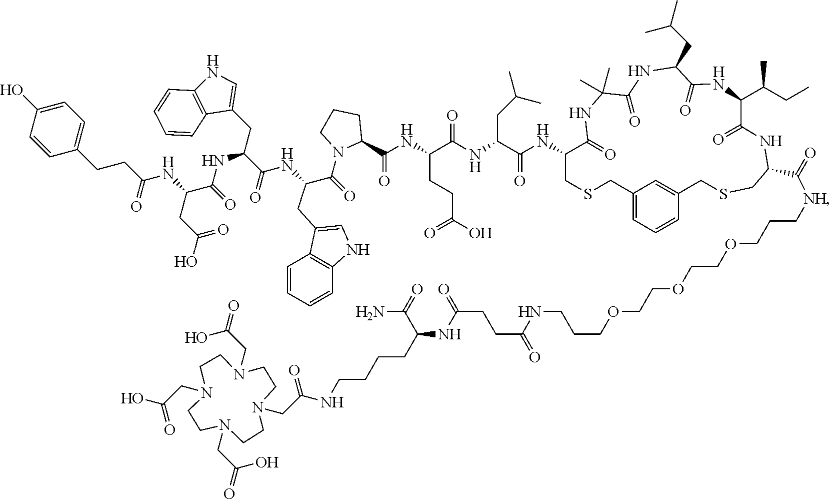 [SMILES: CC[C@H](C)[C@@H]1NC(=O)[C@H](CC(C)C)NC(=O)C(C)(C)NC(=O)[C@@H](NC(=O)[C@@H](CC(C)C)NC(=O)[C@H](CCC(=O)O)NC(=O)[C@@H]2CCCN2C(=O)[C@H](Cc2c[nH]c3ccccc23)NC(=O)[C@H](Cc2c[nH]c3ccccc23)NC(=O)[C@H](CC(=O)O)NC(=O)CCc2ccc(O)cc2)CSCc2cccc(c2)CSC[C@@H](C(=O)NCCCOCCOCCOCCCNC(=O)CCC(=O)N[C@@H](CCCCNC(=O)CN2CCN(CC(=O)O)CCN(CC(=O)O)CCN(CC(=O)O)CC2)C(N)=O)NC1=O]
compound Ac-Asp-Trp-1Ni-Pro-Glu-leu-[Cys(3MeBn)-Ala-Leu-Val-Cys]-Ttds-Lys(DOTA)-NH₂ (GIP-138) of the following formula 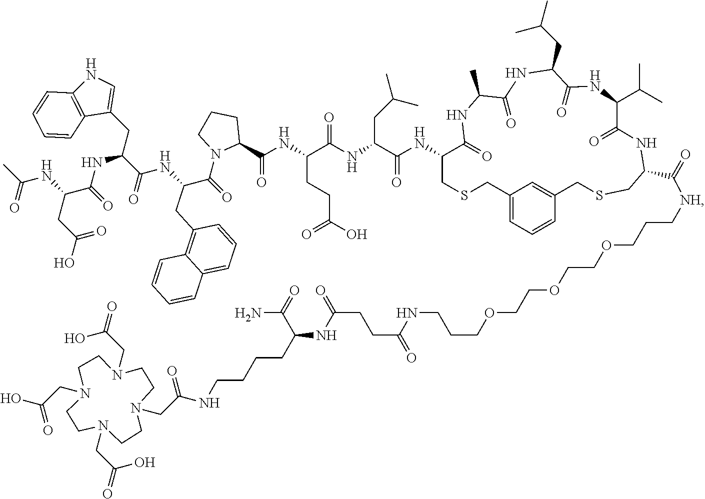 [SMILES: CC(=O)N[C@@H](CC(=O)O)C(=O)N[C@@H](Cc1c[nH]c2ccccc12)C(=O)N[C@@H](Cc1cccc2ccccc12)C(=O)N1CCC[C@H]1C(=O)N[C@@H](CCC(=O)O)C(=O)N[C@H](CC(C)C)C(=O)N[C@H]1CSCc2cccc(c2)CSC[C@@H](C(=O)NCCCOCCOCCOCCCNC(=O)CCC(=O)N[C@@H](CCCCNC(=O)CN2CCN(CC(=O)O)CCN(CC(=O)O)CCN(CC(=O)O)CC2)C(N)=O)NC(=O)[C@H](C(C)C)NC(=O)[C@H](CC(C)C)NC(=O)[C@H](C)NC1=O]
compound Ac-Asp-Trp-1Ni-Pro-Glu-leu-[Cys(3MeBn)-Deg-Leu-Ile-Cys]-Ttds-Lys(DOTA)-NH₂ (GIP-139) of the following formula 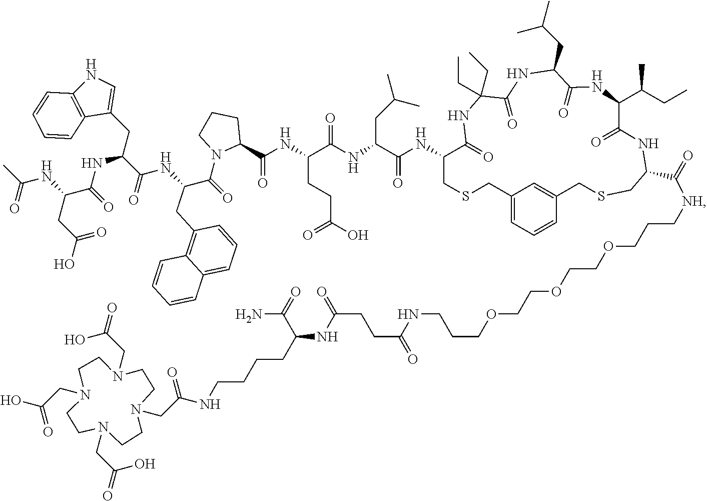 [SMILES: CC[C@H](C)[C@@H]1NC(=O)[C@H](CC(C)C)NC(=O)C(CC)(CC)NC(=O)[C@@H](NC(=O)[C@@H](CC(C)C)NC(=O)[C@H](CCC(=O)O)NC(=O)[C@@H]2CCCN2C(=O)[C@H](Cc2cccc3ccccc23)NC(=O)[C@H](Cc2c[nH]c3ccccc23)NC(=O)[C@H](CC(=O)O)NC(C)=O)CSCc2cccc(c2)CSC[C@@H](C(=O)NCCCOCCOCCOCCCNC(=O)CCC(=O)N[C@@H](CCCCNC(=O)CN2CCN(CC(=O)O)CCN(CC(=O)O)CCN(CC(=O)O)CC2)C(N)=O)NC1=O]
compound Ac-Asp-Trp-6Fw-Pro-Glu-leu-[Cys(3MeBn)-Aib-Leu-Ile-Cys]-Ttds-Lys(DOTA)-NH₂ (GIP-140) of the following formula 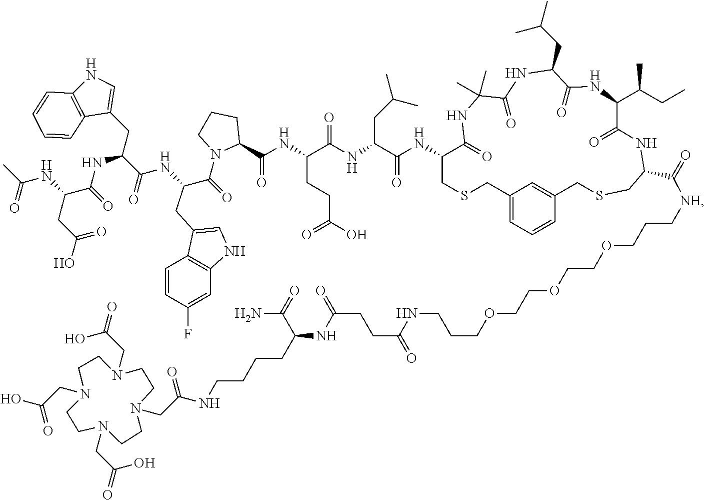 [SMILES: CC[C@H](C)[C@@H]1NC(=O)[C@H](CC(C)C)NC(=O)C(C)(C)NC(=O)[C@@H](NC(=O)[C@@H](CC(C)C)NC(=O)[C@H](CCC(=O)O)NC(=O)[C@@H]2CCCN2C(=O)[C@H](Cc2c[nH]c3cc(F)ccc23)NC(=O)[C@H](Cc2c[nH]c3ccccc23)NC(=O)[C@H](CC(=O)O)NC(C)=O)CSCc2cccc(c2)CSC[C@@H](C(=O)NCCCOCCOCCOCCCNC(=O)CCC(=O)N[C@@H](CCCCNC(=O)CN2CCN(CC(=O)O)CCN(CC(=O)O)CCN(CC(=O)O)CC2)C(N)=O)NC1=O]
compound DOTA-Ttds-Asp-6Clw-Trp-Pro-Glu-leu-[Cys(3MeBn)-Aib-Leu-Ile-AET] (GIP-141) of the following formula 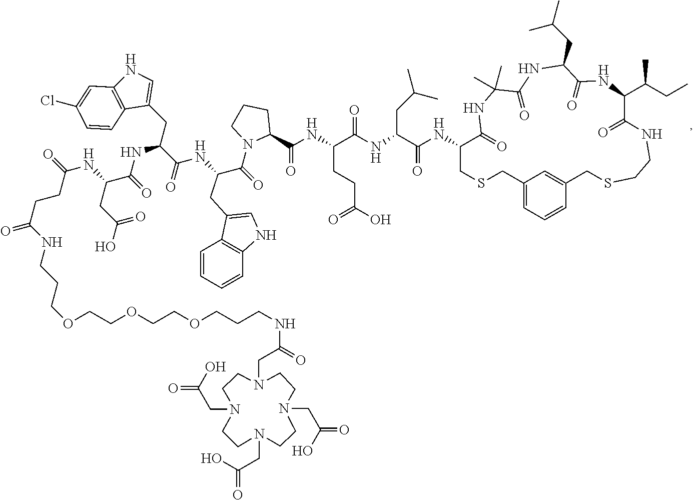 [SMILES: CC[C@H](C)[C@@H]1NC(=O)[C@H](CC(C)C)NC(=O)C(C)(C)NC(=O)[C@@H](NC(=O)[C@@H](CC(C)C)NC(=O)[C@H](CCC(=O)O)NC(=O)[C@@H]2CCCN2C(=O)[C@H](Cc2c[nH]c3ccccc23)NC(=O)[C@H](Cc2c[nH]c3cc(Cl)ccc23)NC(=O)[C@H](CC(=O)O)NC(=O)CCC(=O)NCCCOCCOCCOCCCNC(=O)CN2CCN(CC(=O)O)CCN(CC(=O)O)CCN(CC(=O)O)CC2)CSCc2cccc(c2)CSCCNC1=O]
compound HO-Succinyl-Trp-5Clw-Hyp-Glu-leu-[Cys(2Lut)-Apc(MeO10kDaPEGAc)-Leu-Ile-Cys]-APAc-Lys(DOTA)-NH₂ (GIP-142) of the following formula 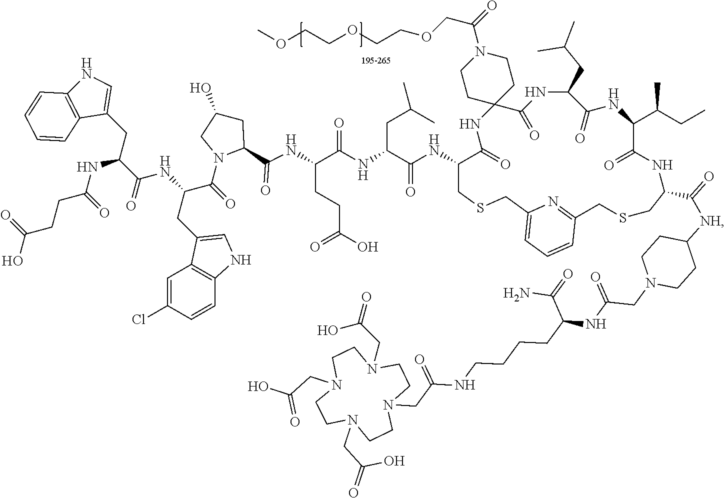
compound HO-Succinyl-Trp-5Clw-Hyp-Glu-leu-[Cys(2Lut)-Apc(MeO2kDaPEGAc)-Leu-Ile-Cys]-APAc-Lys(DOTA)-NH₂ (GIP-143) of the following formula 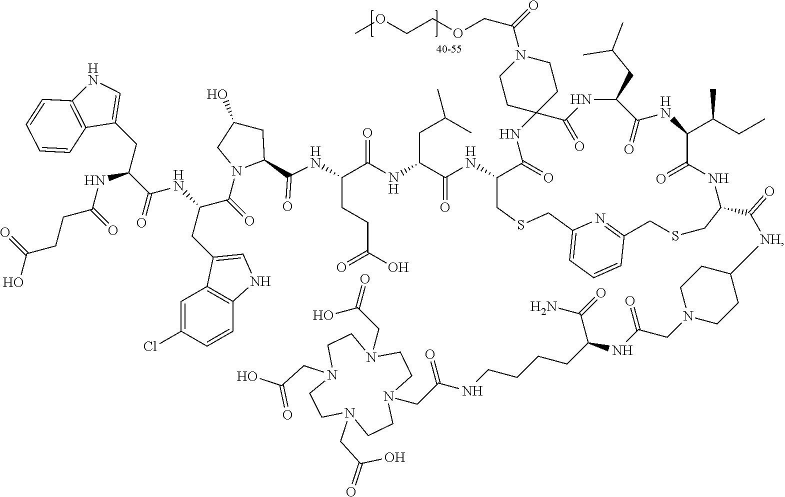
compound HO-Succinyl-Trp-5Clw-Hyp-Glu-leu-[Cys(2Lut)-Thp-Leu-Ile-Cys]-APAc-lys(MeO2kDaPEGAc)-Lys(DOTA)-NH₂ (GIP-144) of the following formula 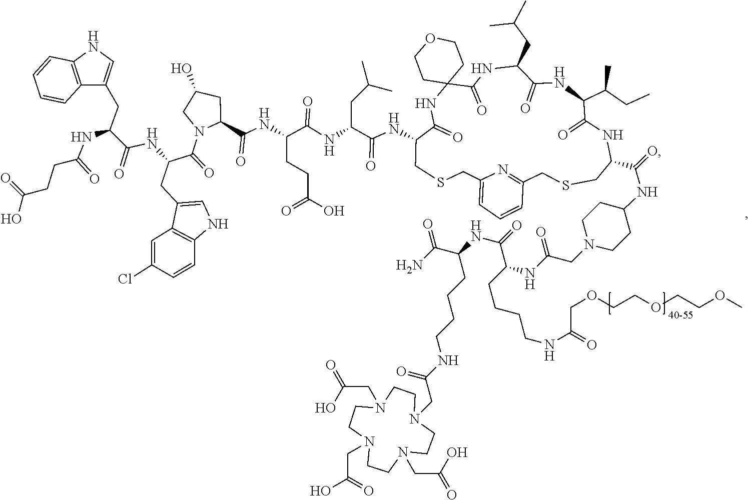
compound Ac-Asp-Trp-5Clw-Hyp-Glu-leu-[Cys(tMeBn (DOTA-PP))-Thp-Leu-Ile-Cys]-NH₂ (GIP-145) of the following formula 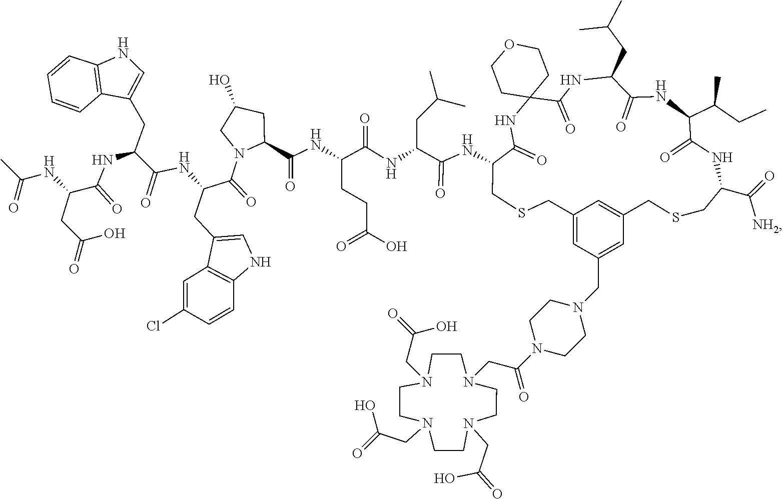 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CC(=O)O)NC(C)=O)CSCc2cc(cc(CN3CCN(C(=O)CN4CCN(CC(=O)O)CCN(CC(=O)O)CCN(CC(=O)O)CC4)CC3)c2)CSC[C@@H](C(N)=O)NC1=O]
compound AGLU-Glutar-Asp-Trp-5Clw-Hyp-Glu-leu-[Cys(2Lut)-Apc(InDOTA)-Leu-Ile-AET] (GIP-146) of the following formula 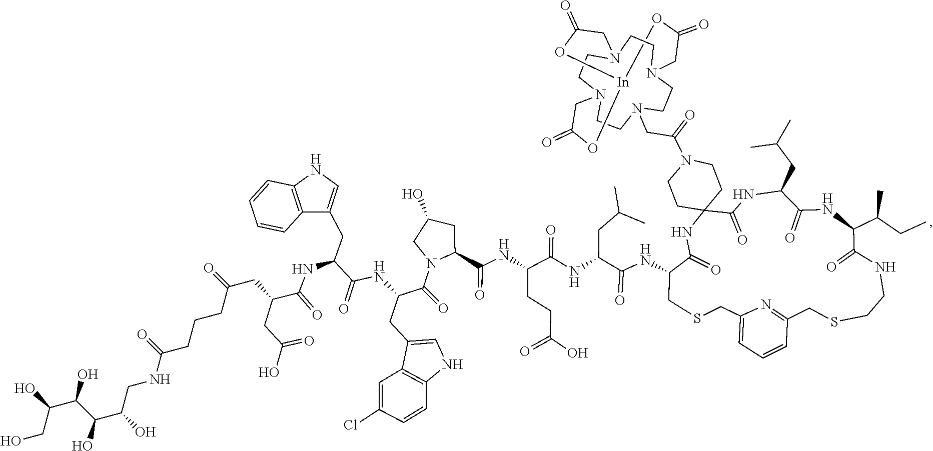
compound AGLU-Glutar-Asp-Trp-5Clw-Hyp-Glu-leu-[Cys(2Lut)-Apc(ZnDOTA)-Leu-Ile-AET] (GIP-147) of the following formula 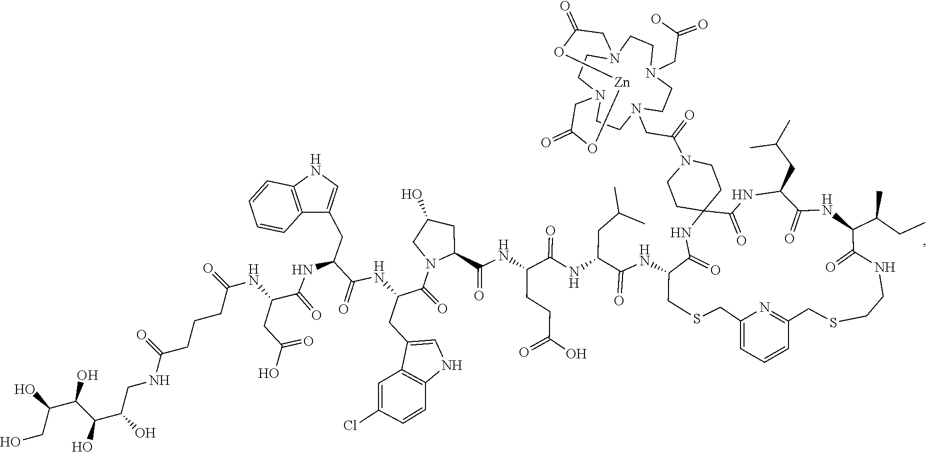
compound InDOTA-Ttds-Asp-Trp-5Clw-Pro-Glu-leu-[Cys(3MeBn)-Aib-Leu-Ile-AET] (GIP-148) of the following formula 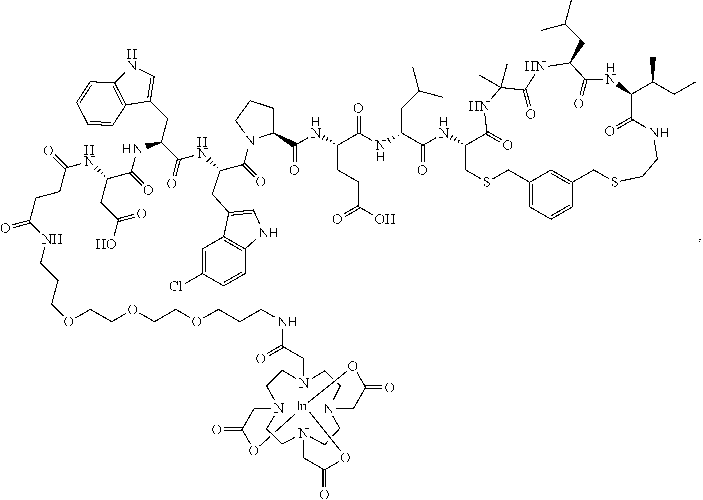
compound InDOTA-Ttds-Asp-Trp-5Clw-Pro-Glu-leu-[Cys(3MeBn)-Ams-Leu-Ile-AET] (GIP-149) of the following formula 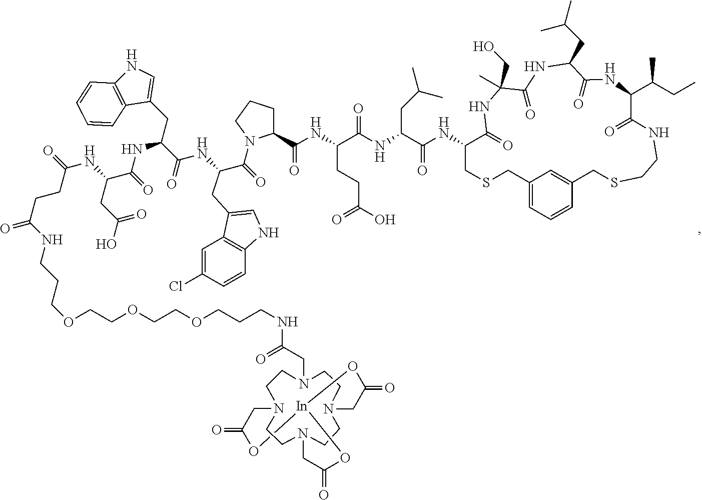
compound InDOTA-Ttds-Asp-Trp-5Clw-Pro-Glu-leu-[Cys(3MeBn)-Thp-Leu-Ile-AET] (GIP-150) of the following formula 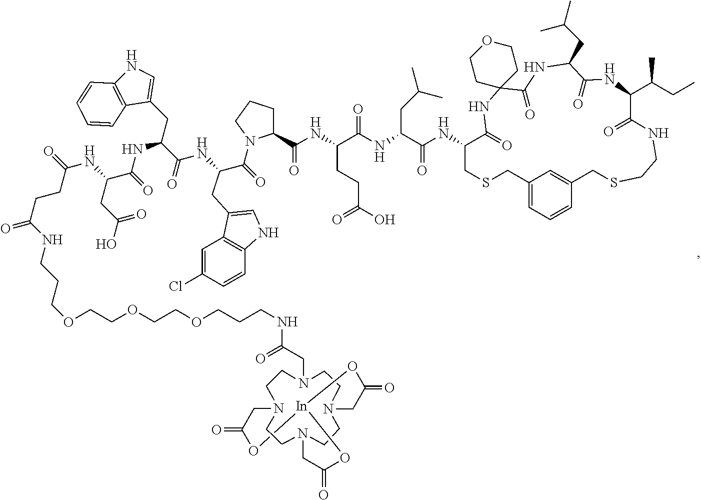
compound InDOTA-Ttds-Asp-Trp-5Clw-Hyp-Glu-leu-[Cys(3MeBn)-Aib-Leu-Ile-AET] (GIP-151) of the following formula 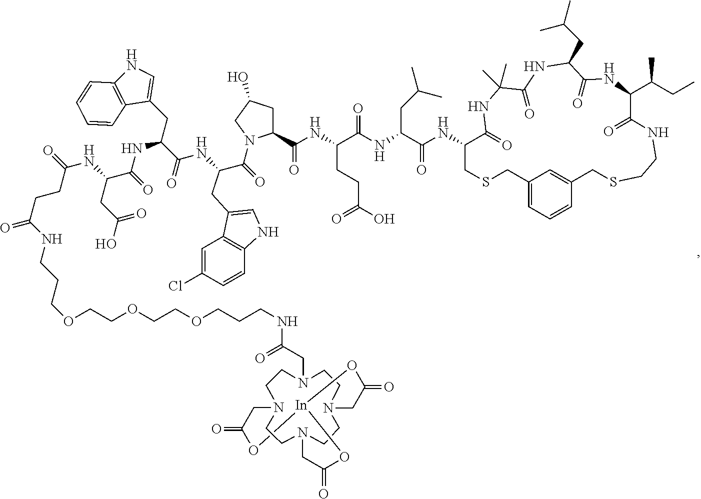
compound InDOTA-Ttds-Asp-Trp-5Clw-Nmg-Glu-leu-[Cys(3MeBn)-Aib-Leu-Ile-AET] (GIP-152) of the following formula 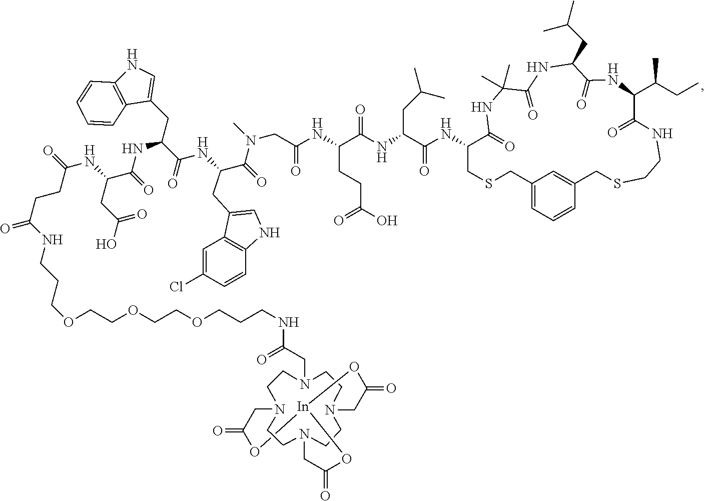
compound InDOTA-Ttds-Asp-Trp-5Clw-Pro-Glu-leu-[Cys(2Lut)-Aib-Leu-Ile-AET] (GIP-153) of the following formula 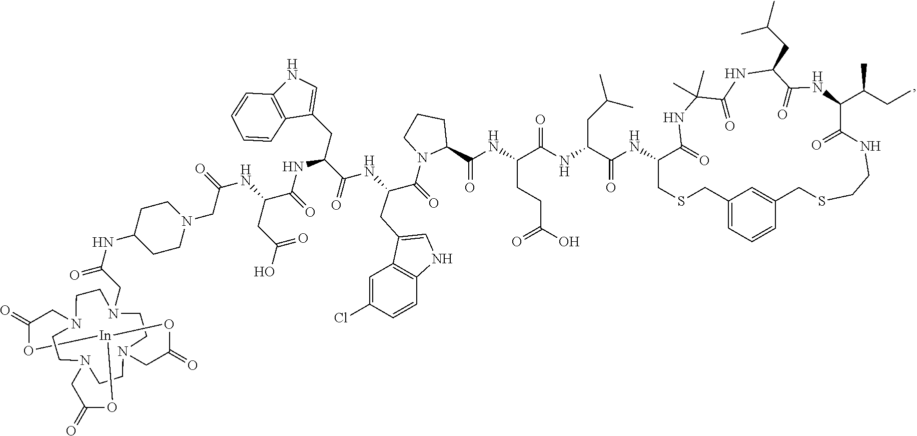
compound InDOTA-Ttds-Asp-Trp-5Clw-Pro-Glu-leu-[Cys(2Lut)-Aib-Leu-Ile-AET] (GIP-154) of the following formula 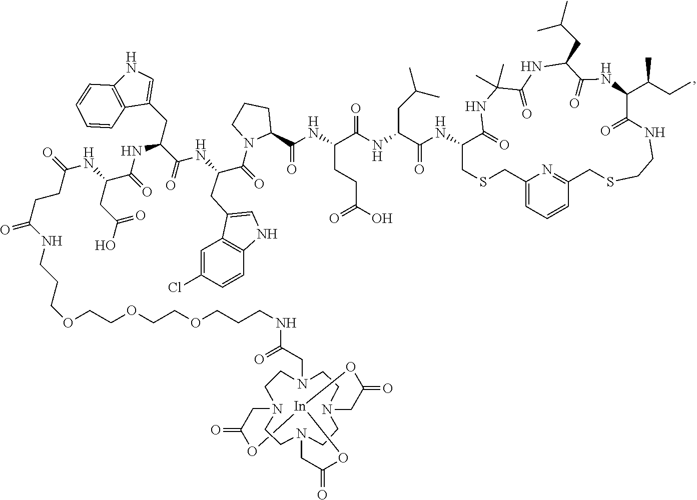
compound InDOTA-APAc-Asp-Trp-5Clw-Hyp-Glu-leu-[Cys(2Lut)-Thp-Leu-Ile-AET] (GIP-155) of the following formula 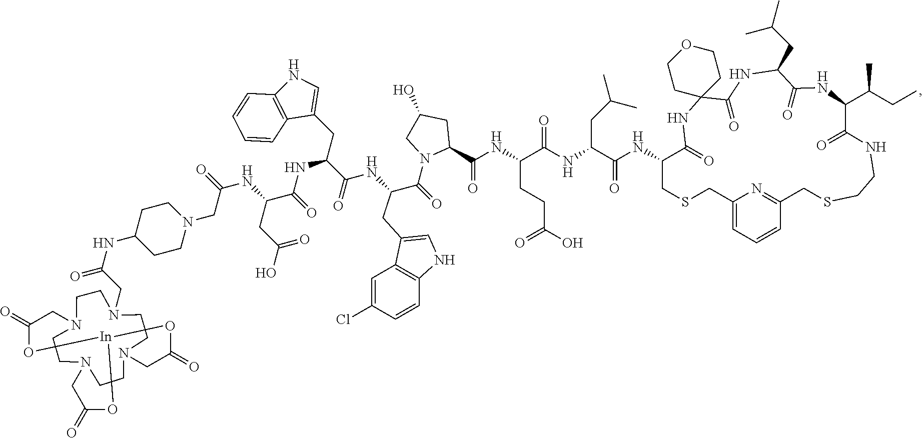
compound Ac-Asp-Trp-5Clw-Pro-Glu-leu-[Cys(3MeBn)-Aib-Leu-Ile-Cys]-Ttds-Lys(LuDOTA)-NH₂ (GIP-156) of the following formula 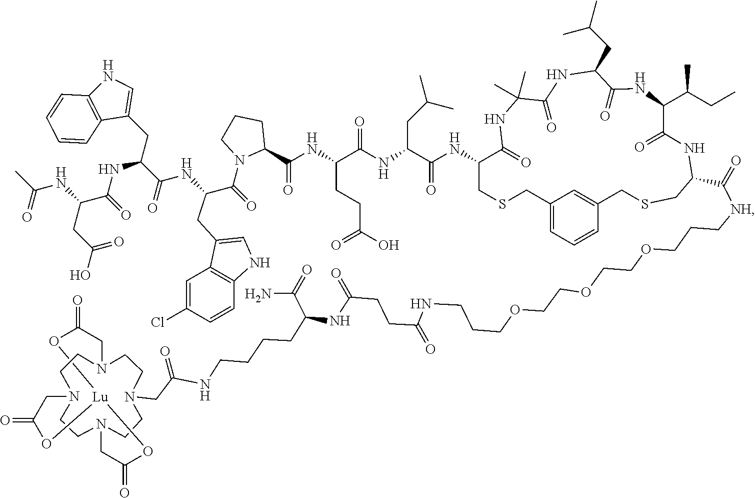
compound LuDOTA-Ttds-Asp-Trp-5Clw-Pro-Glu-leu-[Cys(3MeBn)-Ams-Leu-Ile-AET] (GIP-157) of the following formula 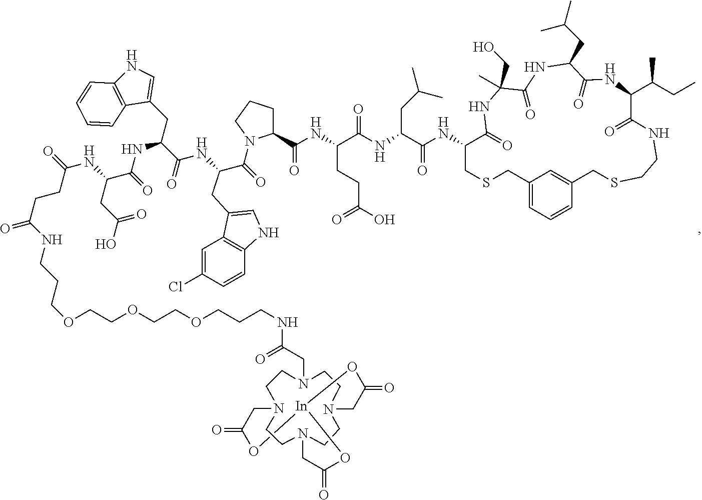
compound LuDOTA-Ttds-Asp-Trp-5Clw-Pro-Glu-leu-[Cys(3MeBn)-Thp-Leu-Ile-AET] (GIP-158) of the following formula 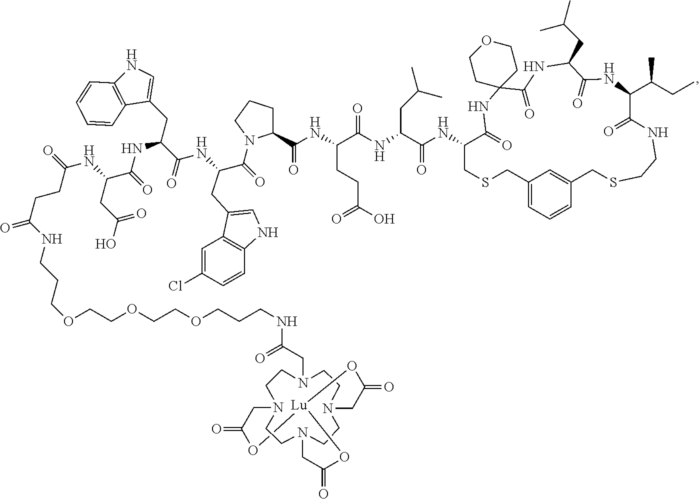
compound LuDOTA-Ttds-Asp-Trp-5Clw-Hyp-Glu-leu-[Cys(3MeBn)-Aib-Leu-Ile-AET] (GIP-159) of the following formula 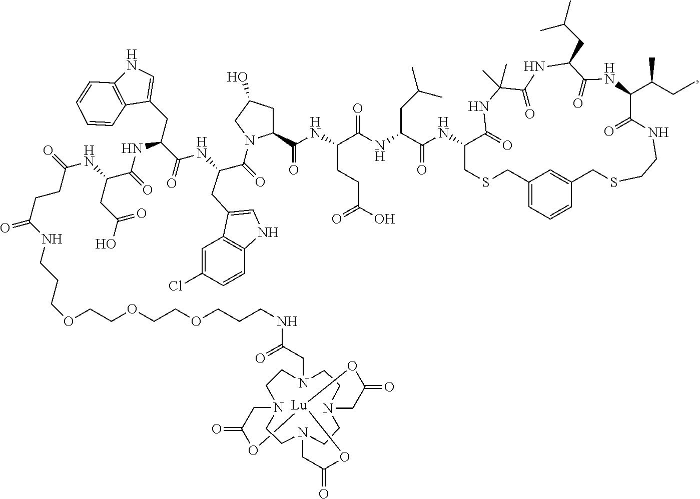
compound LuDOTA-Ttds-Asp-Trp-5Clw-Nmg-Glu-leu-[Cys(3MeBn)-Aib-Leu-Ile-AET] (GIP-160) of the following formula 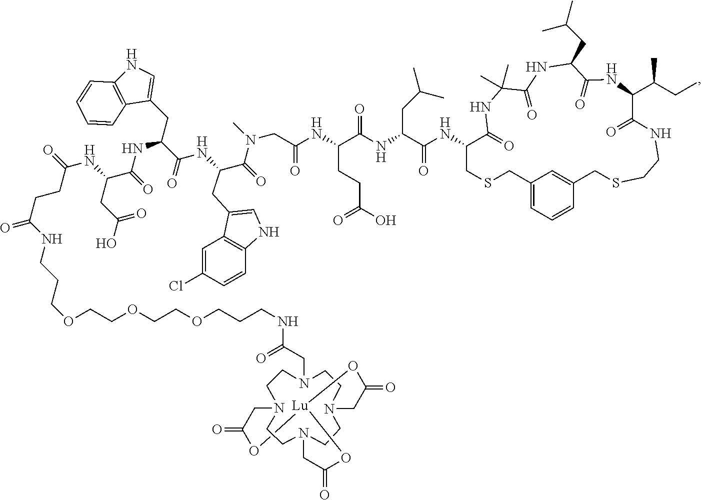
compound LuDOTA-APAc-Asp-Trp-5Clw-Pro-Glu-leu-[Cys(3MeBn)-Aib-Leu-Ile-AET] (GIP-161) of the following formula 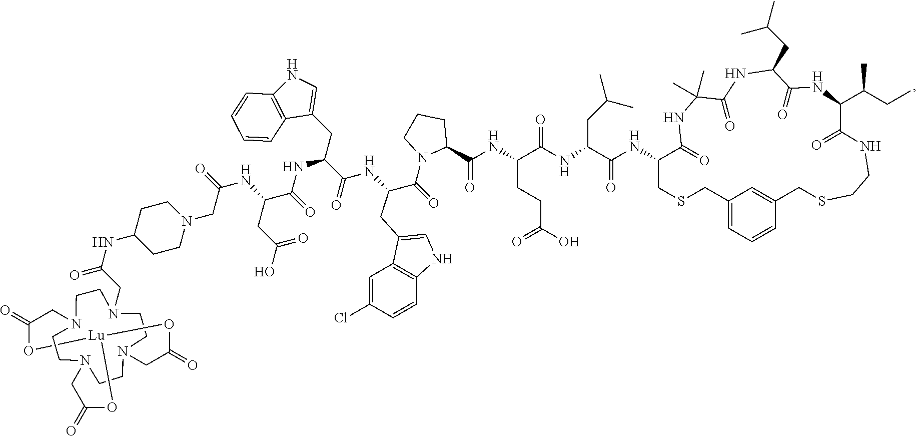
compound LuDOTA-APAc-Asp-Trp-5Clw-Hyp-Glu-leu-[Cys(2Lut)-Thp-Leu-Ile-AET] (GIP-162) of the following formula 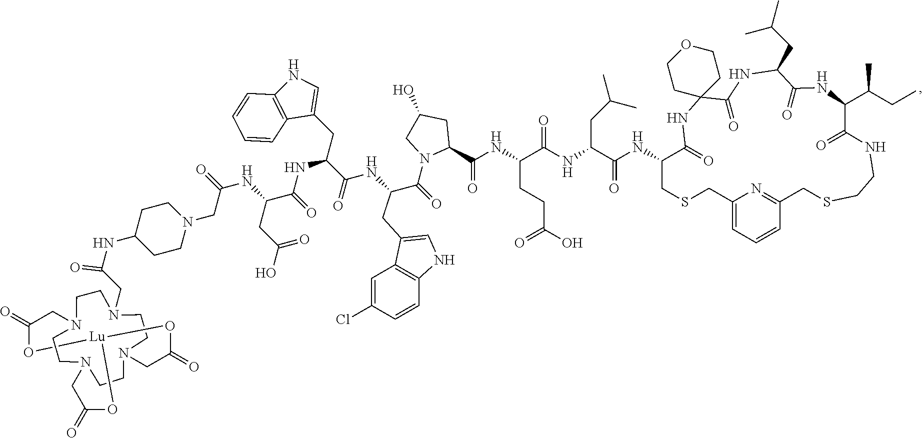
compound LuDOTA-Ttds-Asp-Trp-5Clw-Pro-Glu-leu-[Cys(2Lut)-Aib-Leu-Ile-AET] (GIP-163) of the following formula 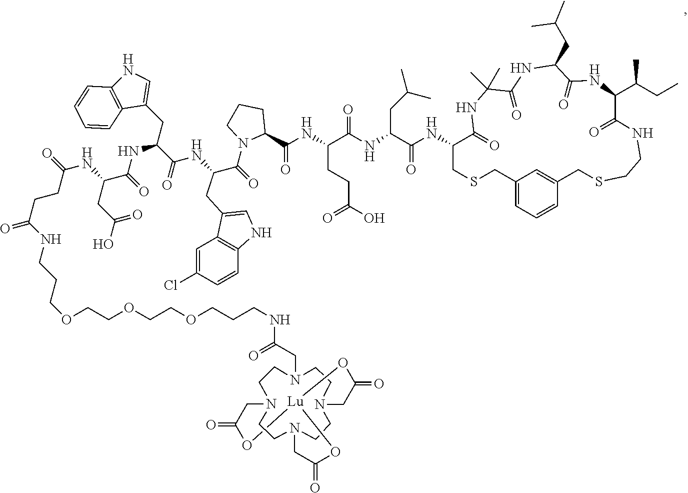
compound Ac-Asp-Trp-5Clw-Pro-Glu-leu-[Cys(3MeBn)-Aib-Leu-Ile-Cys]-Ttds-Lys(InDOTA)-NH₂ (GIP-164) of the following formula 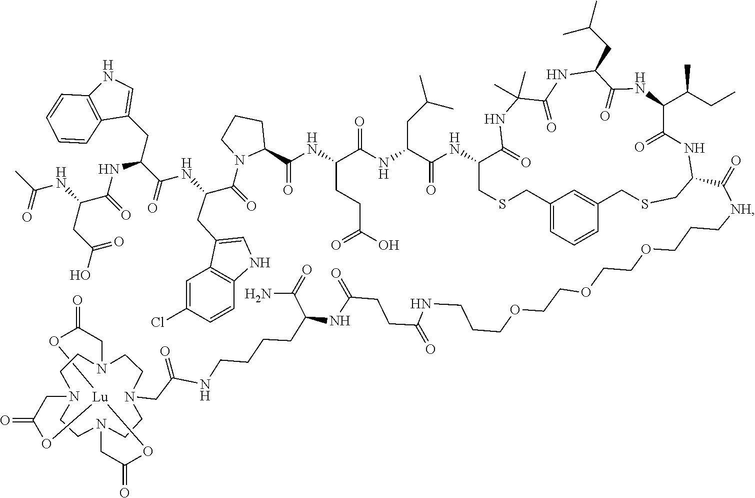
compound Ac-Asp-Trp-1Ni-Pro-Glu-leu-[Cys(3MeBn)-Aib-Leu-Ile-Cys]-Ttds-Lys(InDOTA)-NH₂ (GIP-165) of the following formula 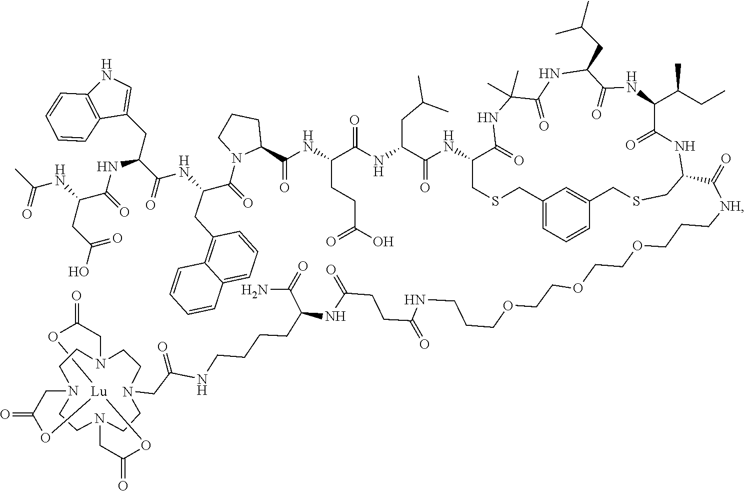
compound Ac-Asp-Trp-1Ni-Pro-Glu-leu-[Cys(3MeBn)-Egz-Leu-Ile-Cys]-Ttds-Lys(InDOTA)-NH₂ (GIP-166) of the following formula 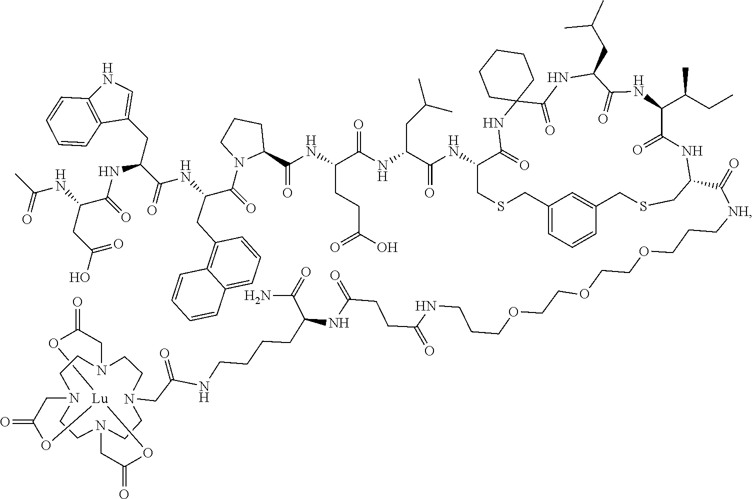
compound LuDOTA-Ttds-Asp-Trp-5Clw-Pro-Glu-leu-[Cys(3MeBn)-Aib-Leu-Ile-AET] (GIP-167) of the following formula 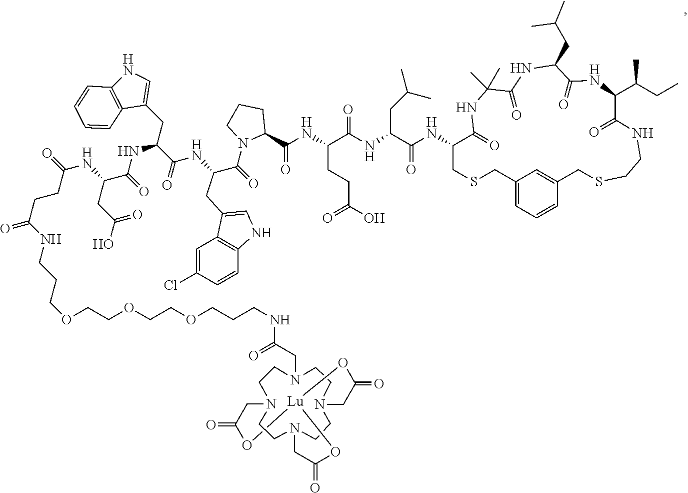
compound HO-Succinyl-Trp-5Clw-Hyp-Glu-leu-[Cys(2Lut)-Thp-Leu-Ile-Cys]-APAc-Lys(InDOTA)-NH₂ (GIP-168) of the following formula 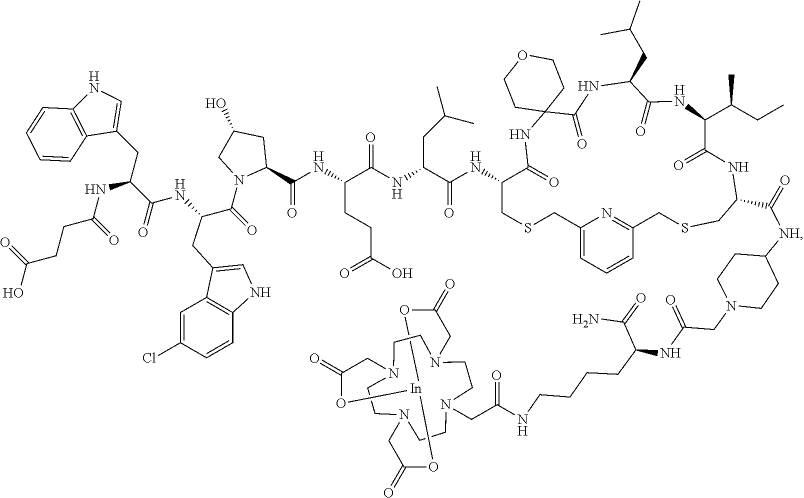
compound HO-Succinyl-Trp-5Clw-Hyp-Glu-leu-[Cys(2Lut)-Apc(InDOTA)-Leu-Ile-AET] (GIP-169) of the following formula 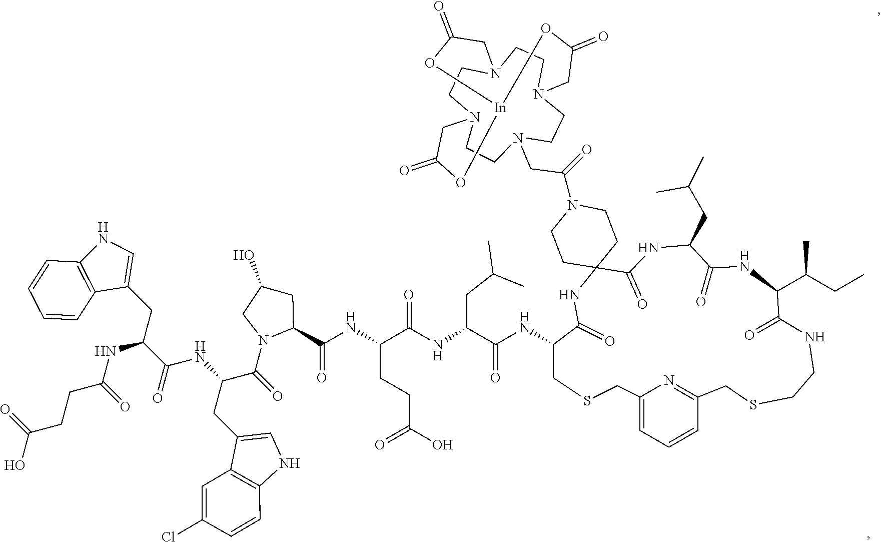
compound HO-Succinyl-Trp-5Clw-Hyp-Glu-leu-[Cys(2Lut)-Thp-Leu-Ile-Cys]-APAc-Lys(LuDOTA)-NH₂ (GIP-170) of the following formula 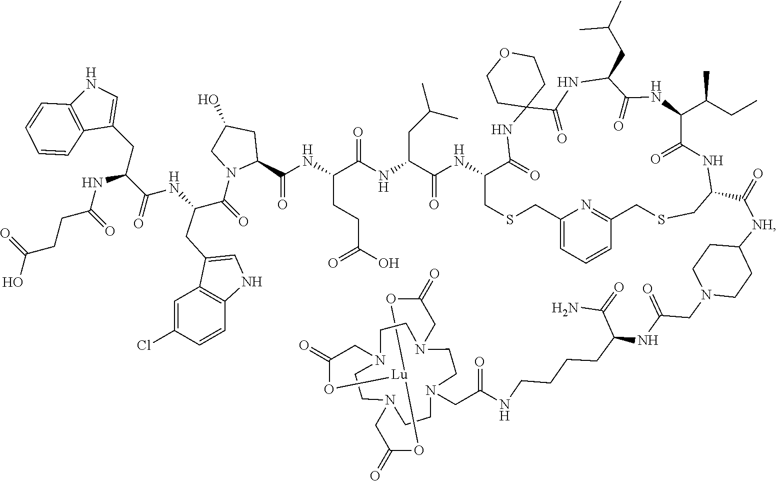
compound HO-Succinyl-Trp-5Clw-Hyp-Glu-leu-[Cys(2Lut)-Apc(LuDOTA)-Leu-Ile-AET] (GIP-171) of the following formula 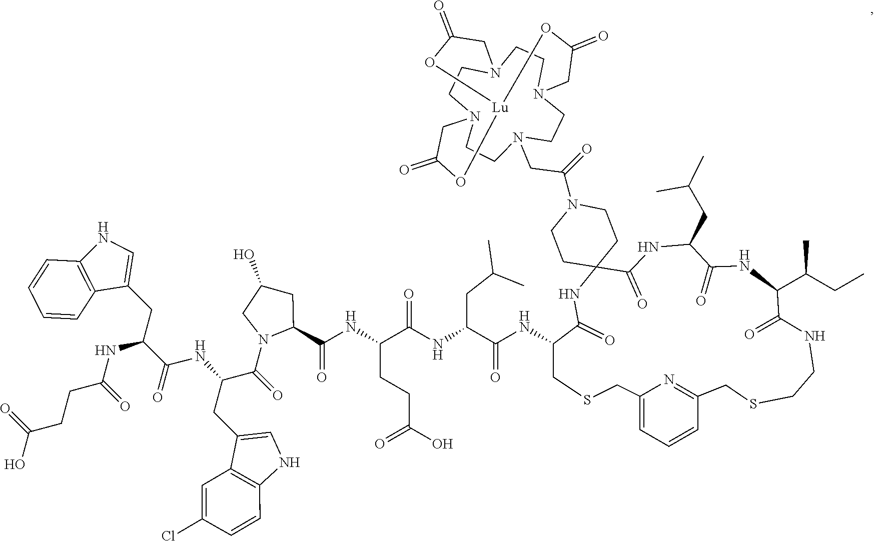
compound Ac-Asp-Trp-1Ni-Pro-Glu-leu-[Cys(3MeBn)-Aib-Leu-Ile-Cys]-Ttds-Lys(LuDOTA)-NH₂ (GIP-172) of the following formula 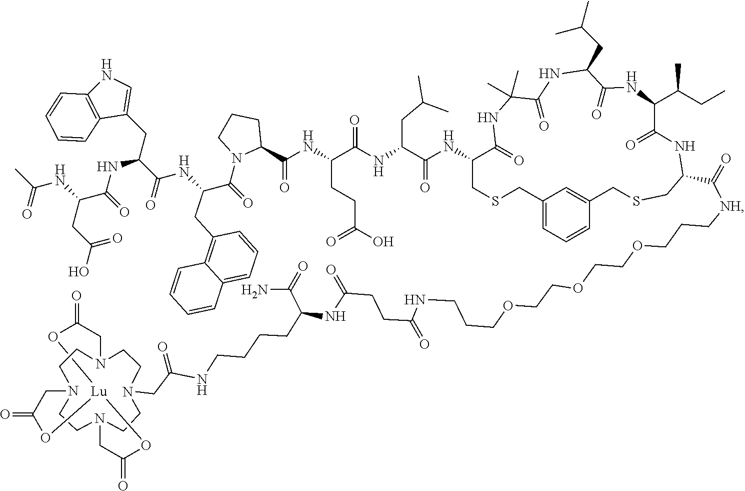
compound Ac-Asp-Trp-1Ni-Pro-Glu-leu-[Cys(3MeBn)-Egz-Leu-Ile-Cys]-Ttds-Lys(LuDOTA)-NH₂ (GIP-173) of the following formula 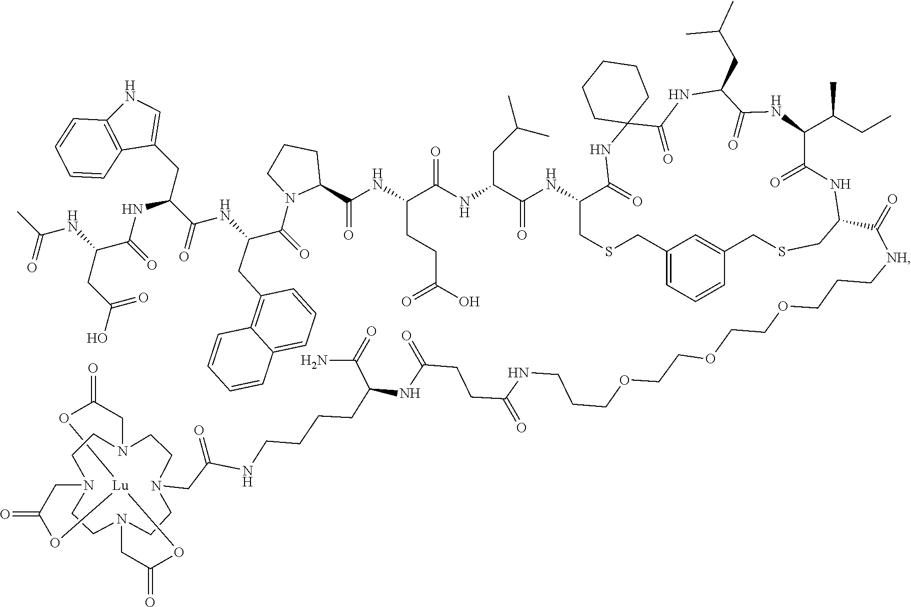
compound AGLU-Glutar-Asp-Trp-5Clw-Hyp-Glu-leu-[Cys(2Lut)-Thp-Leu-Ile-Cys]-APAc-glu(AGLU)-NH₂ (GIP-174) of the following formula 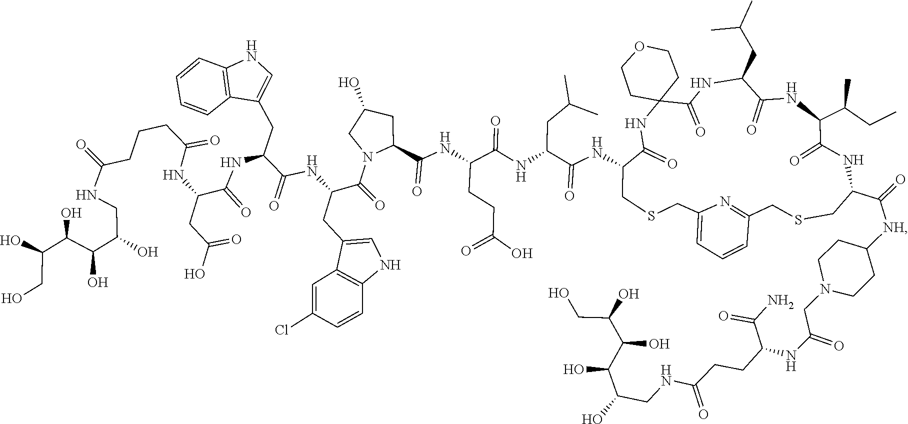 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CC(=O)O)NC(=O)CCCC(=O)NC[C@H](O)[C@@H](O)[C@H](O)[C@H](O)CO)CSCc2cccc(n2)CSC[C@@H](C(=O)NC2CCN(CC(=O)N[C@H](CCC(=O)NC[C@H](O)[C@@H](O)[C@H](O)[C@H](O)CO)C(N)=O)CC2)NC1=O]
compound Ac-Asp-Trp-5Clw-Tap (AGLU-Glutar)-Glu-leu-[Cys(2Lut)-Thp-Leu-Ile-Cys]-NH₂ (GIP-175) of the following formula 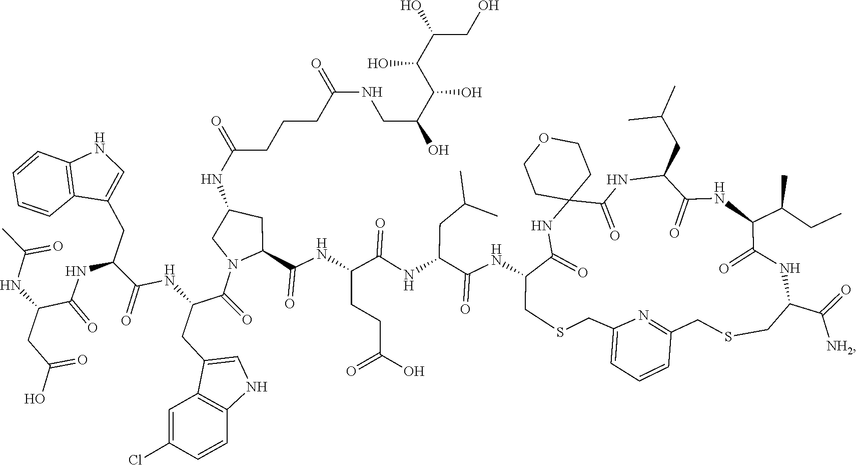 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](NC(=O)CCCC(=O)NC[C@H](O)[C@@H](O)[C@H](O)[C@H](O)CO)CN2C(=O)[C@H](Cc2c[nH]c3ccc(Cl)cc23)NC(=O)[C@H](Cc2c[nH]c3ccccc23)NC(=O)[C@H](CC(=O)O)NC(C)=O)CSCc2cccc(n2)CSC[C@@H](C(N)=O)NC1=O]
compound Ac-Asp-Trp-5Clw-Nglu(AGLU)-Glu-leu-[Cys(2Lut)-Thp-Leu-Ile-Cys]-NH₂ (GIP-176) of the following formula 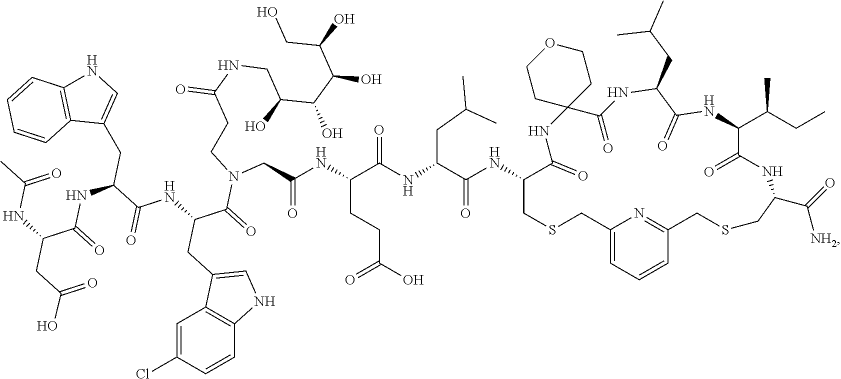
compound Ac-Asp-Trp-5Clw-Hyp-Glu-leu-[Cys(2Lut)-Thp-Leu-Ile-Cys]-APAc-glu(AGLU)-NH₂ (GIP-177) of the following formula 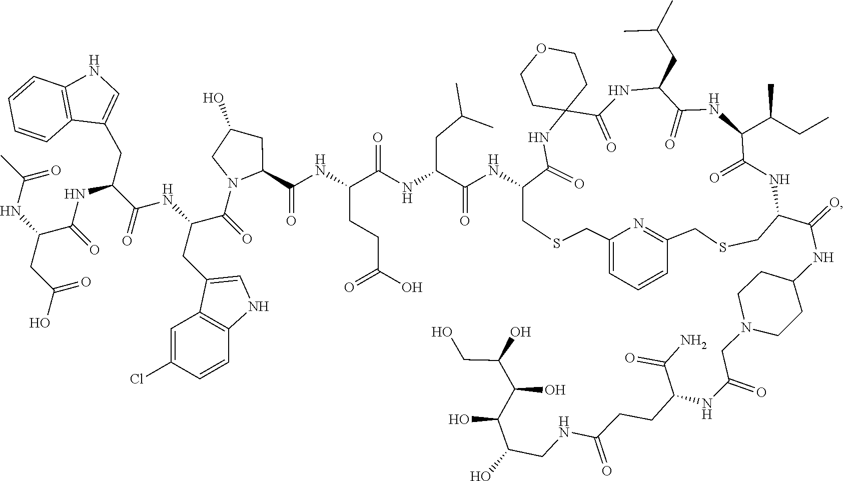 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CC(=O)O)NC(C)=O)CSCc2cccc(n2)CSC[C@@H](C(=O)NC2CCN(CC(=O)N[C@H](CCC(=O)NC[C@H](O)[C@@H](O)[C@H](O)[C@H](O)CO)C(N)=O)CC2)NC1=O]
compound AGLU-Glutar-Asp-Trp-5Clw-Hyp-Glu-leu-[Cys(2Lut)-Thp-Leu-Ile-Cys]-NH₂ (GIP-178) of the following formula 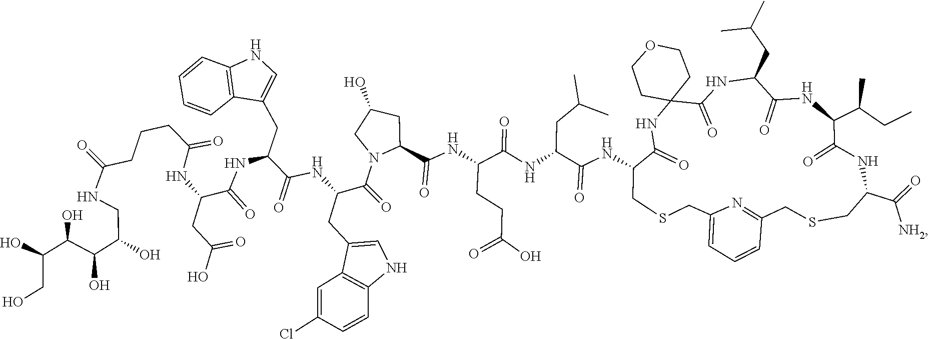 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CC(=O)O)NC(=O)CCCC(=O)NC[C@H](O)[C@@H](O)[C@H](O)[C@H](O)CO)CSCc2cccc(n2)CSC[C@@H](C(N)=O)NC1=O]
compound Ac-Cya-Trp-5Clw-Hyp-Glu-leu-[Cys(2Lut)-Aib-Leu-Ile-AET] (GIP-179) of the following formula 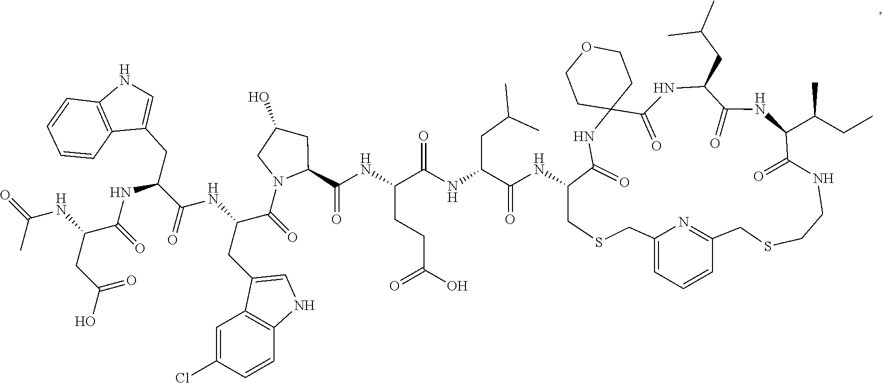 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CC(=O)O)NC(C)=O)CSCc2cccc(n2)CSCCNC1=O]
compound Ac-Asp-Trp-5Clw-Hyp-Glu-leu-[Cys(2Lut)-Thp-Leu-Ile-Cys]-NH₂ (GIP-180) of the following formula 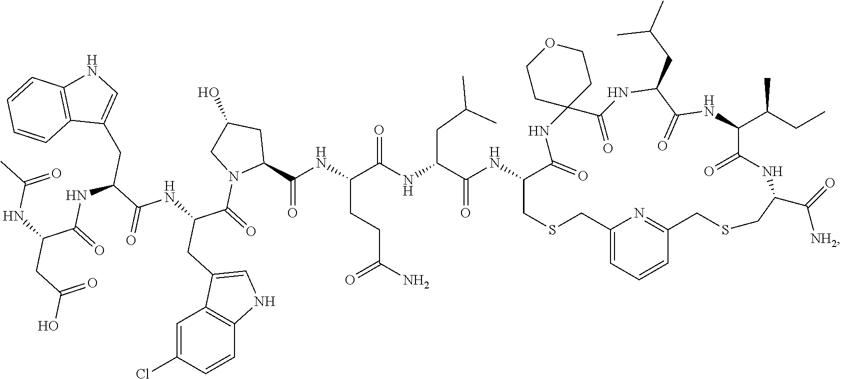 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(N)=O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CC(=O)O)NC(C)=O)CSCc2cccc(n2)CSC[C@@H](C(N)=O)NC1=O]
compound Ac-Asp-Trp-5Clw-Hyp-Glu-leu-[Cys(2Lut)-Thp-Leu-Ile-Cys]-OH (GIP-181) of the following formula 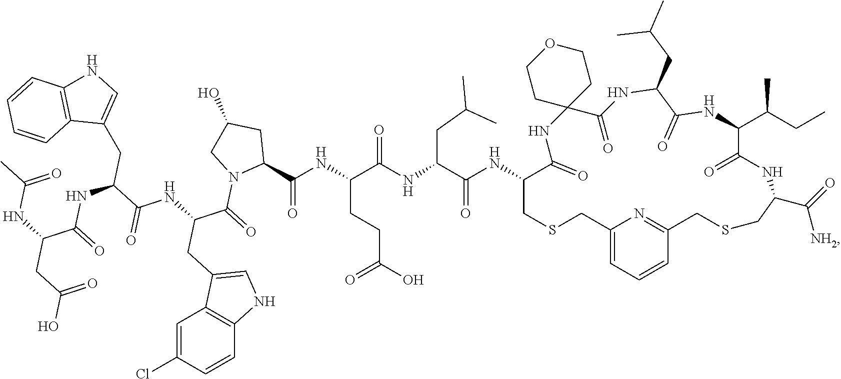 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CC(=O)O)NC(C)=O)CSCc2cccc(n2)CSC[C@@H](C(N)=O)NC1=O]
compound Ac-Asp-Trp-5Clw-Hyp-Glu-leu-[Cys(2Lut)-Thp-Leu-Ile-Cysol]-OH (GIP-182) of the following formula 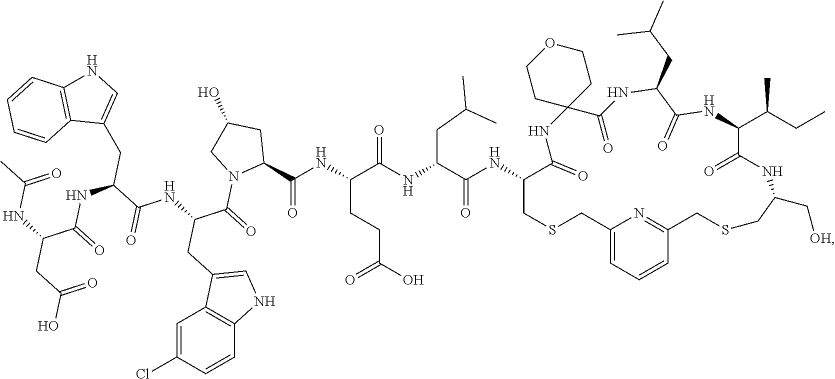 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CC(=O)O)NC(C)=O)CSCc2cccc(n2)CSC[C@@H](CO)NC1=O]
compound Ac-Asp-Trp-5Clw-Nmg-Glu-leu-[Cys(2Lut)-Thp-Leu-Ile-Cys]-NH₂ (GIP-183) of the following formula 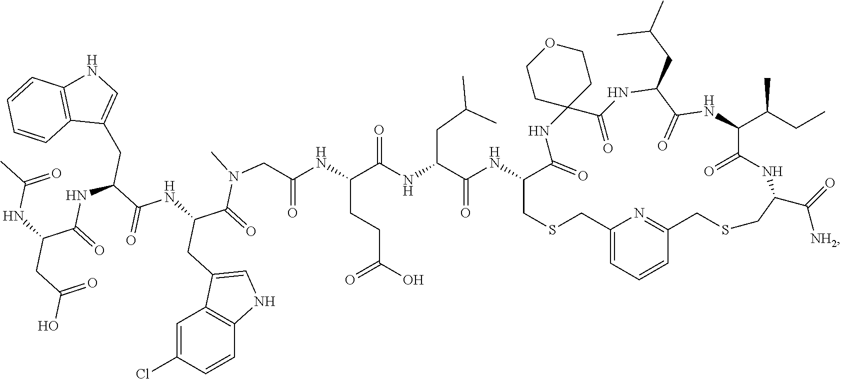 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)CN(C)C(=O)[C@H](Cc2c[nH]c3ccc(Cl)cc23)NC(=O)[C@H](Cc2c[nH]c3ccccc23)NC(=O)[C@H](CC(=O)O)NC(C)=O)CSCc2cccc(n2)CSC[C@@H](C(N)=O)NC1=O]
compound Ac-Asp-Trp-5Clw-Nleu-Glu-leu-[Cys(2Lut)-Thp-Leu-Ile-Cys]-NH₂ (GIP-184) of the following formula 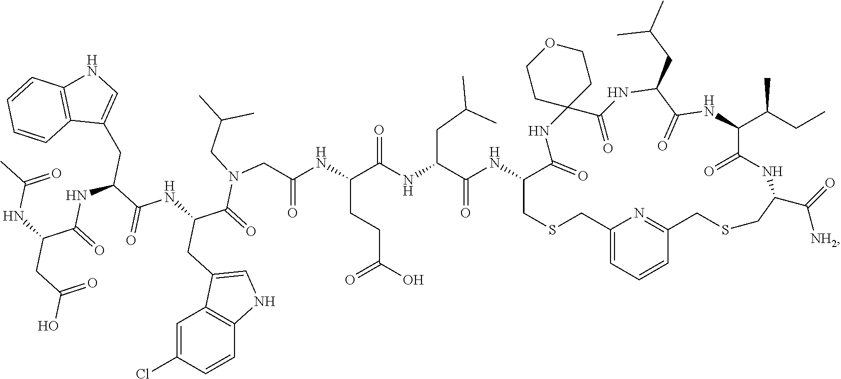 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)CN(CC(C)C)C(=O)[C@H](Cc2c[nH]c3ccc(Cl)cc23)NC(=O)[C@H](Cc2c[nH]c3ccccc23)NC(=O)[C@H](CC(=O)O)NC(C)=O)CSCc2cccc(n2)CSC[C@@H](C(N)=O)NC1=O]
compound Ac-Asp-Trp-5Clw-Nphe-Glu-leu-[Cys(2Lut)-Thp-Leu-Ile-Cys]-NH₂ (GIP-185) of the following formula 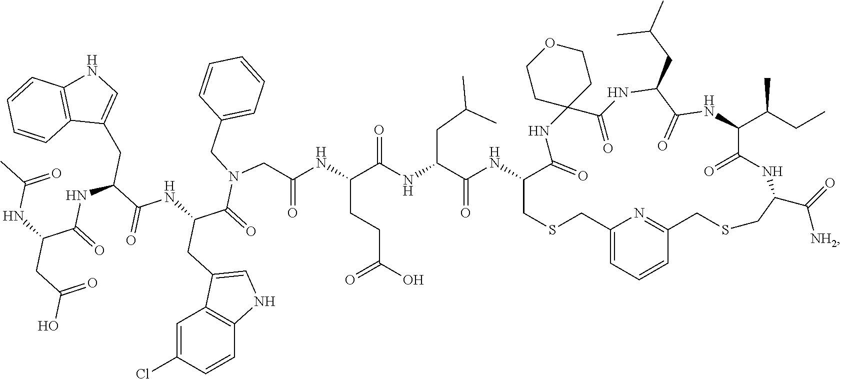 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)CN(Cc2ccccc2)C(=O)[C@H](Cc2c[nH]c3ccc(Cl)cc23)NC(=O)[C@H](Cc2c[nH]c3ccccc23)NC(=O)[C@H](CC(=O)O)NC(C)=O)CSCc2cccc(n2)CSC[C@@H](C(N)=O)NC1=O]
compound Ac-Asp-Trp-5Clw-Nlys-Glu-leu-[Cys(2Lut)-Thp-Leu-Ile-Cys]-NH₂ (GIP-186) of the following formula 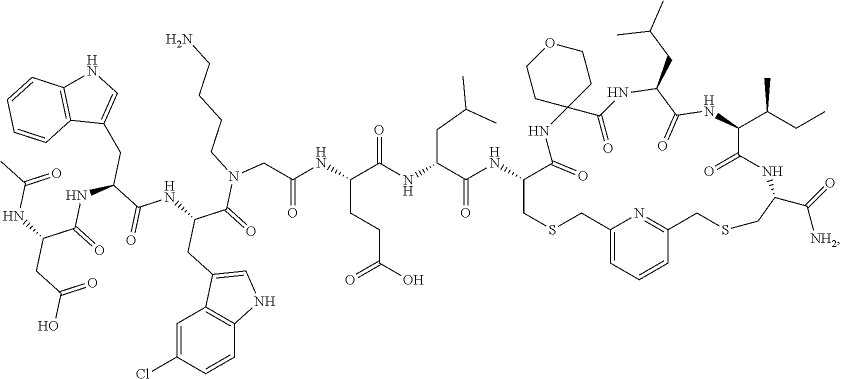 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)CN(CCCCN)C(=O)[C@H](Cc2c[nH]c3ccc(Cl)cc23)NC(=O)[C@H](Cc2c[nH]c3ccccc23)NC(=O)[C@H](CC(=O)O)NC(C)=O)CSCc2cccc(n2)CSC[C@@H](C(N)=O)NC1=O]
compound Ac-Asp-Trp-5Clw-4Tfp-Glu-leu-[Cys(2Lut)-Thp-Leu-Ile-Cys]-NH₂ (GIP-187) of the following formula 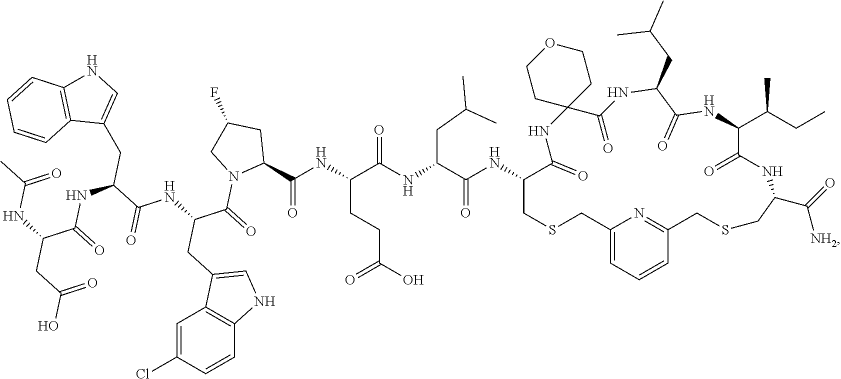 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](F)CN2C(=O)[C@H](Cc2c[nH]c3ccc(Cl)cc23)NC(=O)[C@H](Cc2c[nH]c3ccccc23)NC(=O)[C@H](CC(=O)O)NC(C)=O)CSCc2cccc(n2)CSC[C@@H](C(N)=O)NC1=O]
compound Ac-Ser-Trp-5Clw-Hyp-Glu-leu-[Cys(2Lut)-Thp-Leu-Ile-Cys]-NH₂ (GIP-188) of the following formula 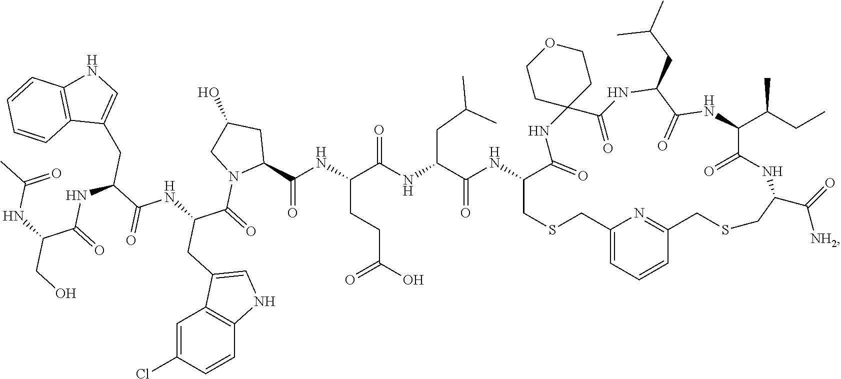 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CO)NC(C)=O)CSCc2cccc(n2)CSC[C@@H](C(N)=O)NC1=O]
compound Ac-Cya-Trp-5Clw-Hyp-Glu-leu-[Cys(2Lut)-Thp-Leu-Ile-Cys]-NH₂ (GIP-189) of the following formula 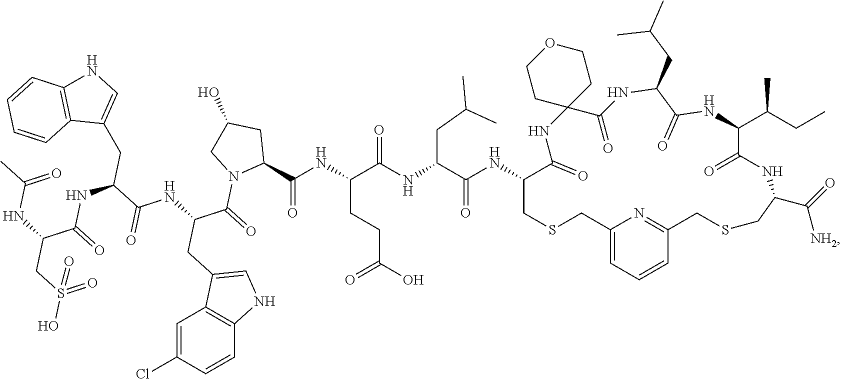 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CS(=O)(=O)O)NC(C)=O)CSCc2cccc(n2)CSC[C@@H](C(N)=O)NC1=O]
compound Ac-Asn-Trp-5Clw-Hyp-Glu-leu-[Cys(2Lut)-Thp-Leu-Ile-Cys]-NH₂ (GIP-190) of the 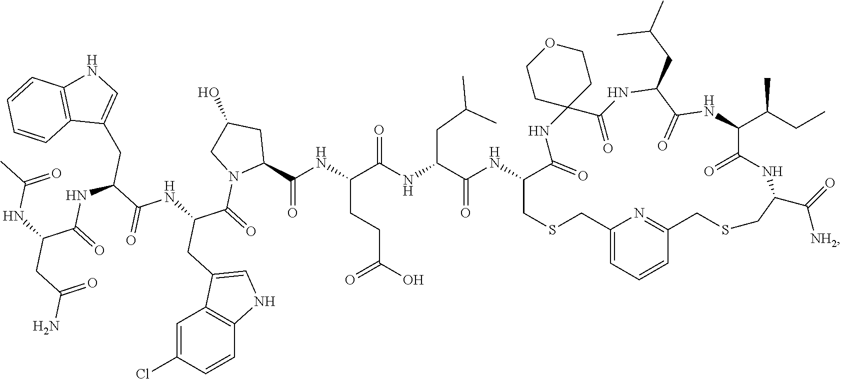 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CC(N)=O)NC(C)=O)CSCc2cccc(n2)CSC[C@@H](C(N)=O)NC1=O]
compound Ac-Glu-Trp-5Clw-Hyp-Glu-leu-[Cys(2Lut)-Thp-Leu-Ile-Cys]-NH₂ (GIP-191) of the following formula 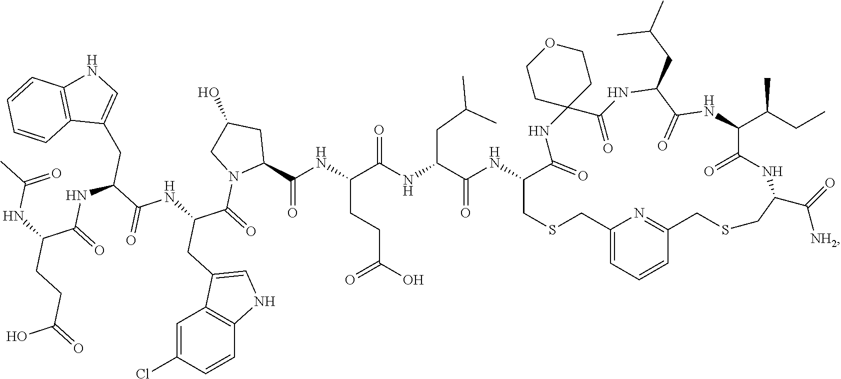 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CCC(=O)O)NC(C)=O)CSCc2cccc(n2)CSC[C@@H](C(N)=O)NC1=O]
compound Ac-Asp-Trp-5Clw-Hyp-Asp-leu-[Cys(2Lut)-Thp-Leu-Ile-Cys]-NH₂ (GIP-192) of the following formula 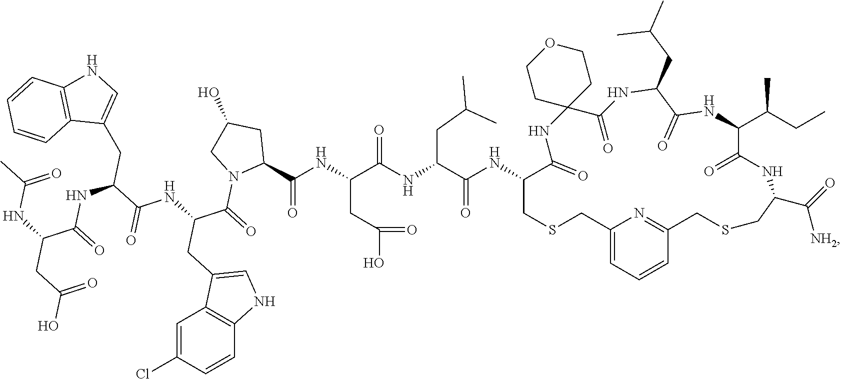 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CC(=O)O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CC(=O)O)NC(C)=O)CSCc2cccc(n2)CSC[C@@H](C(N)=O)NC1=O]
compound Ac-Asp-Trp-5Clw-Hyp-Gln-leu-[Cys(2Lut)-Thp-Leu-Ile-Cys]-NH₂ (GIP-193) of the following formula 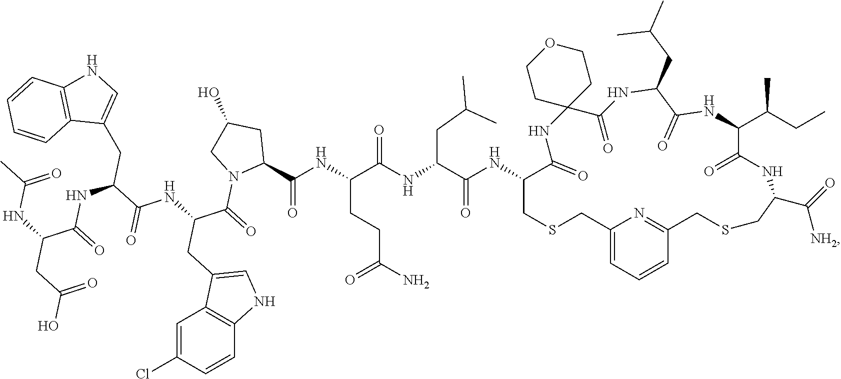 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(N)=O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CC(=O)O)NC(C)=O)CSCc2cccc(n2)CSC[C@@H](C(N)=O)NC1=O]
compound Ac-Asp-Trp-5Clw-Hyp-Hse-leu-[Cys(2Lut)-Thp-Leu-Ile-Cys]-NH₂ (GIP-194) of the 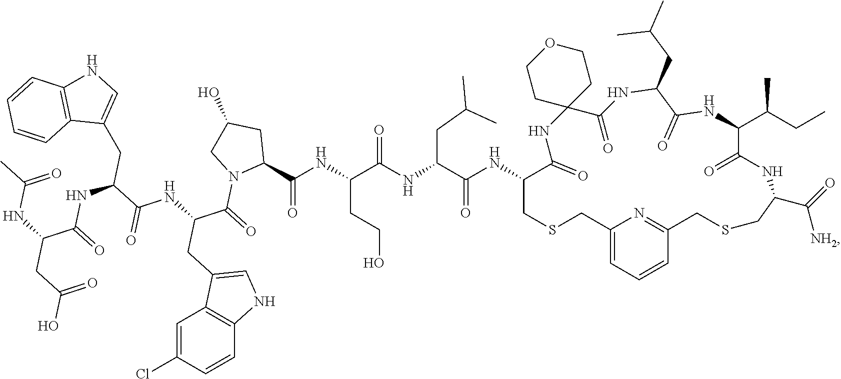 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCO)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CC(=O)O)NC(C)=O)CSCc2cccc(n2)CSC[C@@H](C(N)=O)NC1=O]
compound Ac-Hse-Trp-5Clw-Hyp-Glu-leu-[Cys(2Lut)-Thp-Leu-Ile-Cys]-NH₂ (GIP-195) of the following formula 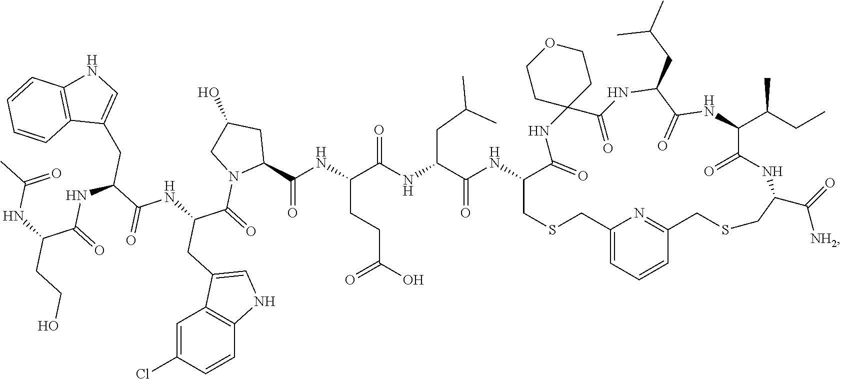 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CCO)NC(C)=O)CSCc2cccc(n2)CSC[C@@H](C(N)=O)NC1=O]
compound Ac-Asp-Trp-5Clw-Nglu-Glu-leu-[Cys(2Lut)-Thp-Leu-Ile-Cys]-NH₂ (GIP-196) of the following formula 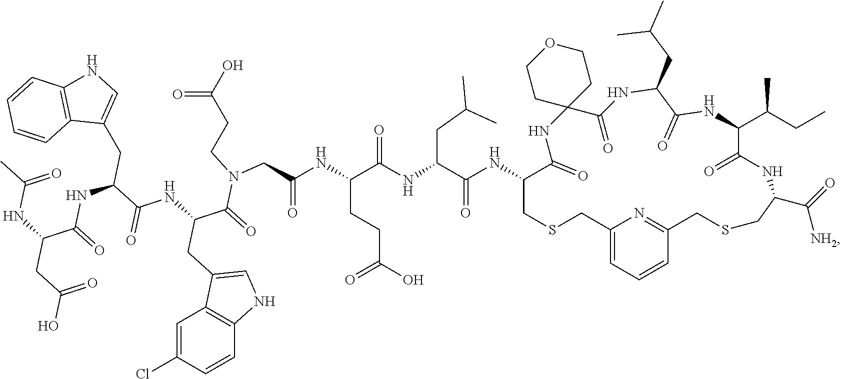
compound Ac-Hse-Trp-5Brw-Hyp-Glu-leu-[Cys(2Lut)-Thp-Leu-Ile-Cys]-NH₂ (GIP-197) of the following formula 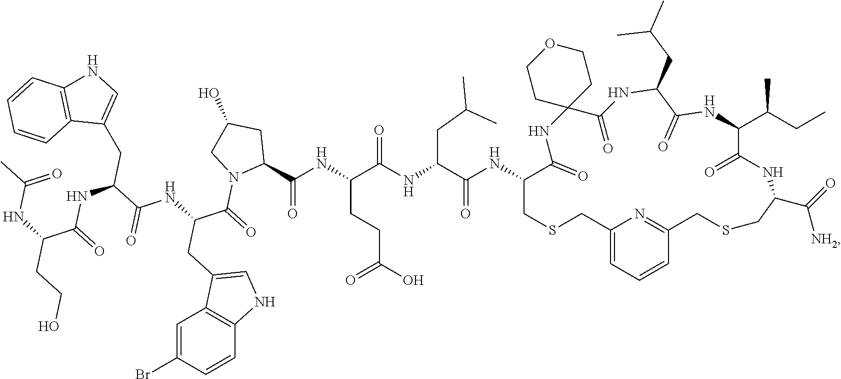 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Br)cc23)NC(=O)[C@H](Cc2c[nH]c3ccccc23)NC(=O)[C@H](CCO)NC(C)=O)CSCc2cccc(n2)CSC[C@@H](C(N)=O)NC1=O]
compound HO-Succinyl-Trp-5Clw-Pro-Glu-leu-[Cys(3MeBn)-Aib-Leu-Ile-Cys]-NH₂ (GIP-198) of the following formula 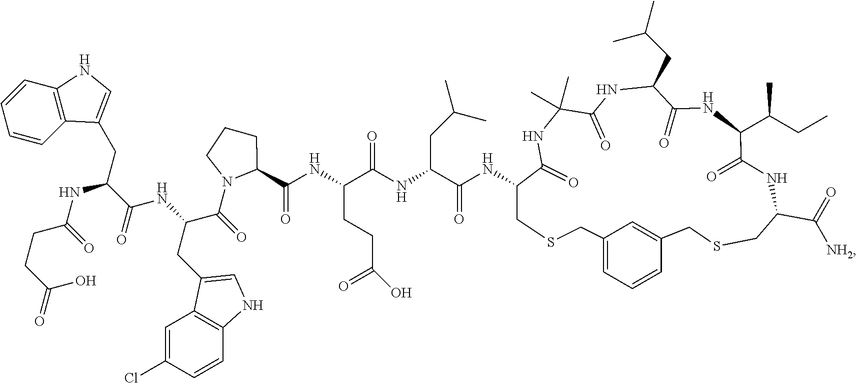 [SMILES: CC[C@H](C)[C@@H]1NC(=O)[C@H](CC(C)C)NC(=O)C(C)(C)NC(=O)[C@@H](NC(=O)[C@@H](CC(C)C)NC(=O)[C@H](CCC(=O)O)NC(=O)[C@@H]2CCCN2C(=O)[C@H](Cc2c[nH]c3ccc(Cl)cc23)NC(=O)[C@H](Cc2c[nH]c3ccccc23)NC(=O)CCC(=O)O)CSCc2cccc(c2)CSC[C@@H](C(N)=O)NC1=O]
compound HO-Succinyl-Trp-5Clw-Pro-Glu-leu-[Cys(3MeBn)-Thp-Leu-Ile-Cys]-NH₂ (GIP-199) of the following formula 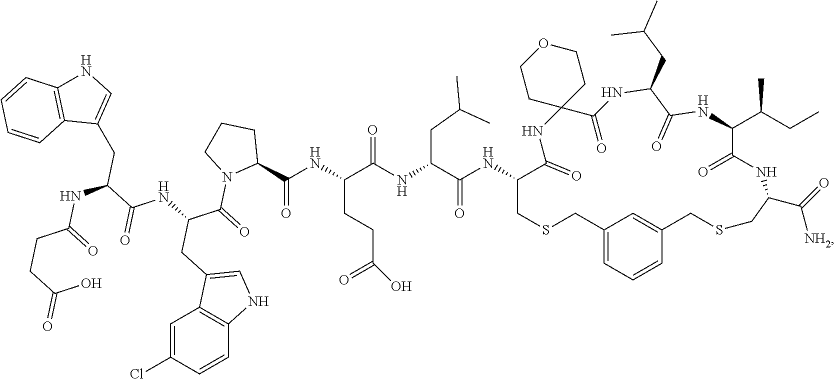 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2CCCN2C(=O)[C@H](Cc2c[nH]c3ccc(Cl)cc23)NC(=O)[C@H](Cc2c[nH]c3ccccc23)NC(=O)CCC(=O)O)CSCc2cccc(c2)CSC[C@@H](C(N)=O)NC1=O]
compound Ac-Asp-Trp-5Clw-Hyp-Glu-leu-[Hcy(2Lut)-Thp-Leu-Ile-Cys]-NH₂ (GIP-200) of the following formula 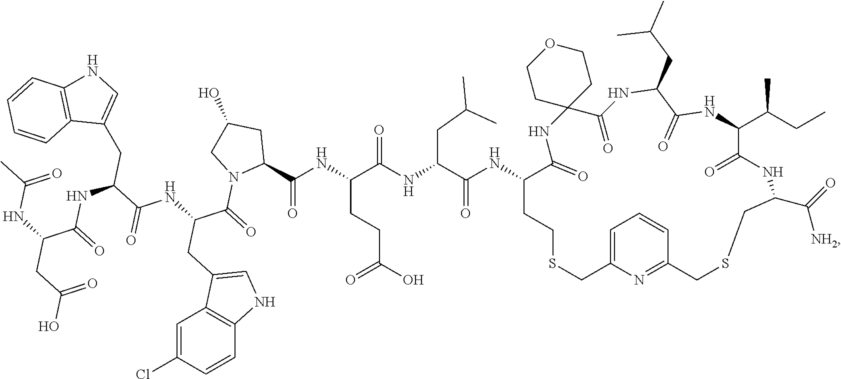 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CC(=O)O)NC(C)=O)CCSCc2cccc(n2)CSC[C@@H](C(N)=O)NC1=O]
compound Ac-Asp-Trp-5Clw-Hyp-Glu-leu-[Cys(2Lut)-Thp-Leu-Ile-Hcy]-NH₂ (GIP-201) of the following formula 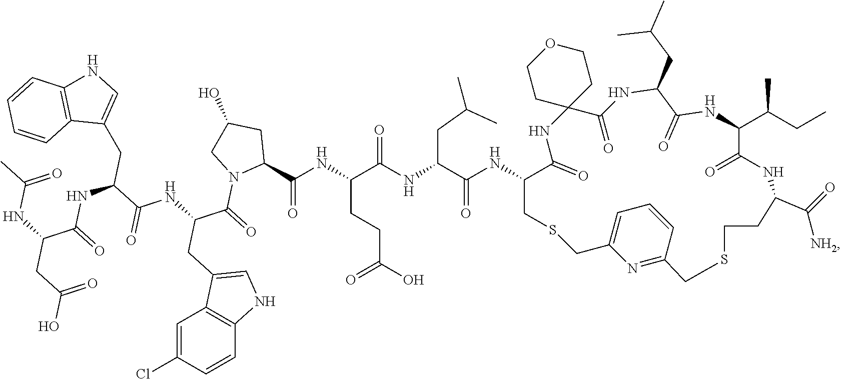 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CC(=O)O)NC(C)=O)CSCc2cccc(n2)CSCC[C@@H](C(N)=O)NC1=O]
compound Ac-Asp-Trp-5Clw-Hyp-Glu-leu-[Hcy(2Lut)-Thp-Leu-Ile-Hcy]-NH₂ (GIP-202) of the following formula 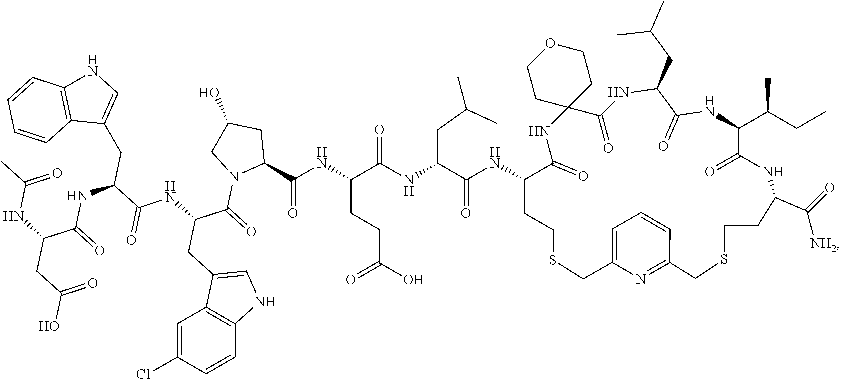 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CC(=O)O)NC(C)=O)CCSCc2cccc(n2)CSCC[C@@H](C(N)=O)NC1=O]
compound Ac-Asp-Trp-5Clw-Hyp-Glu-leu-[Cys(2Lut)-Eca-Leu-Ile-Cys]-NH₂ (GIP-203) of the following formula 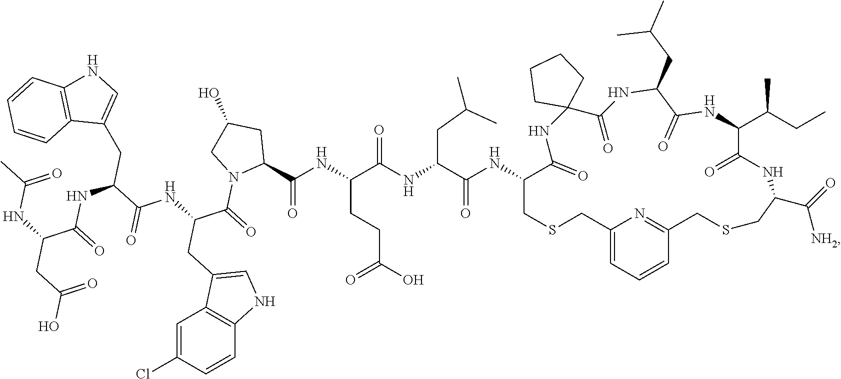 [SMILES: CC[C@H](C)[C@@H]1NC(=O)[C@H](CC(C)C)NC(=O)C2(CCCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CC(=O)O)NC(C)=O)CSCc2cccc(n2)CSC[C@@H](C(N)=O)NC1=O]
compound Ac-Gln-Trp-5Clw-Hyp-Glu-leu-[Cys(2Lut)-Thp-Leu-Ile-Cys]-NH₂ (GIP-204) of the following formula 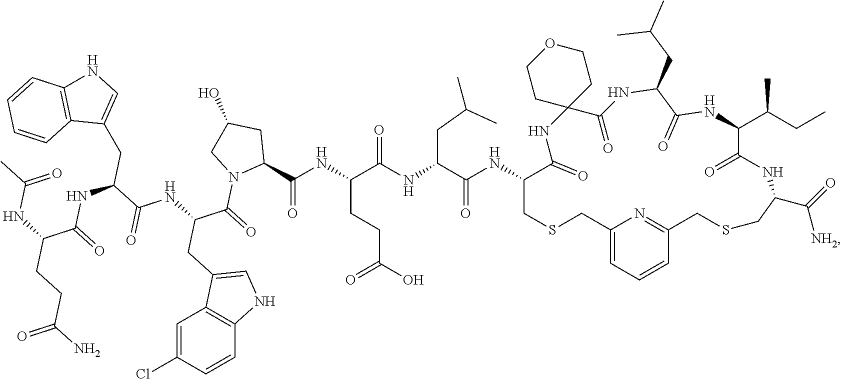 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CCC(N)=O)NC(C)=O)CSCc2cccc(n2)CSC[C@@H](C(N)=O)NC1=O]
compound Ac-Asp-Trp-5Clw-Hyp-Asn-leu-[Cys(2Lut)-Thp-Leu-Ile-Cys]-NH₂ (GIP-205) of the following formula 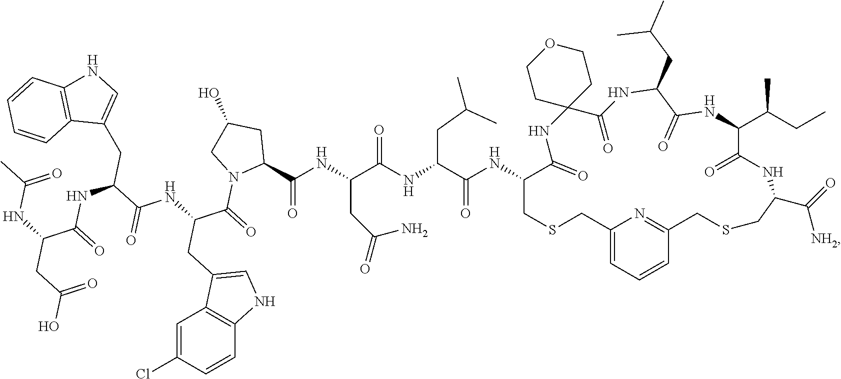 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CC(N)=O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CC(=O)O)NC(C)=O)CSCc2cccc(n2)CSC[C@@H](C(N)=O)NC1=O]
compound Ac-Asp-Trp-5Clw-Hyp-Glu-leu-[Cys(2Lut)-Aic-Leu-Ile-Cys]-NH₂ (GIP-206) of the 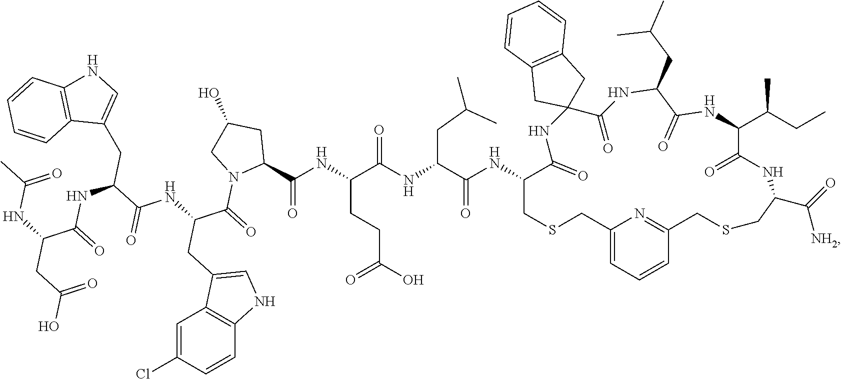 [SMILES: CC[C@H](C)[C@@H]1NC(=O)[C@H](CC(C)C)NC(=O)C2(Cc3ccccc3C2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CC(=O)O)NC(C)=O)CSCc2cccc(n2)CSC[C@@H](C(N)=O)NC1=O]
compound Ac-Pse-Trp-5Clw-Hyp-Glu-leu-[Cys(2Lut)-Thp-Leu-Ile-Cys]-NH₂ (GIP-207) of the following formula 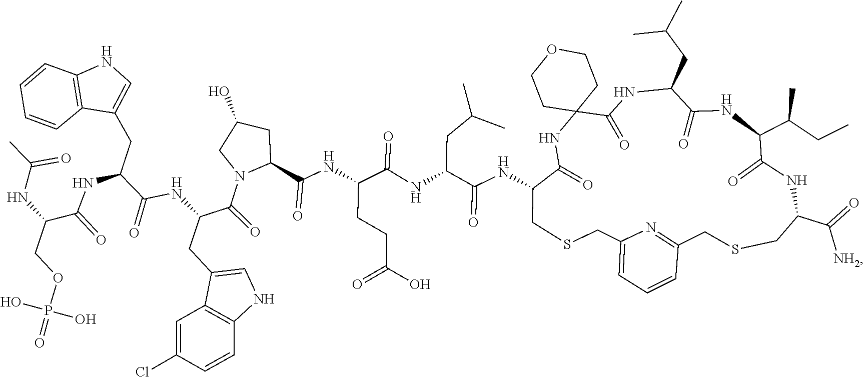 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](COP(=O)(O)O)NC(C)=O)CSCc2cccc(n2)CSC[C@@H](C(N)=O)NC1=O]
compound Ac-Asp-Trp-5Clw-Hyp-Pse-leu-[Cys(2Lut)-Thp-Leu-Ile-Cys]-NH₂ (GIP-208) of the following formula 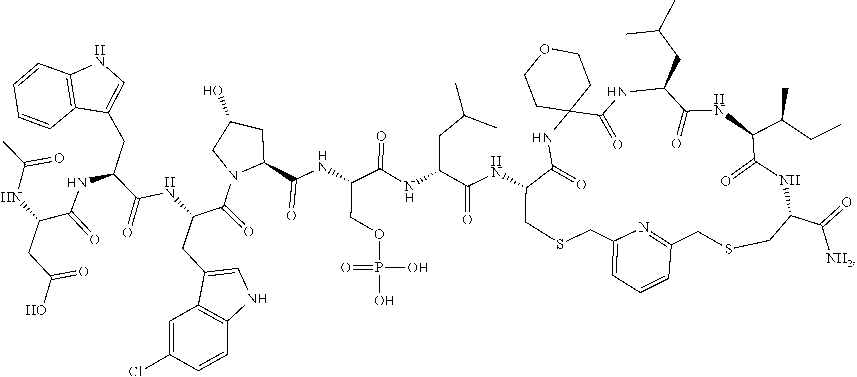 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](COP(=O)(O)O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CC(=O)O)NC(C)=O)CSCc2cccc(n2)CSC[C@@H](C(N)=O)NC1=O]
compound Ac-Asp-Mcf-5Clw-Hyp-Glu-leu-[Cys(2Lut)-Thp-Leu-Ile-Cys]-NH₂ (GIP-209) of the following formula 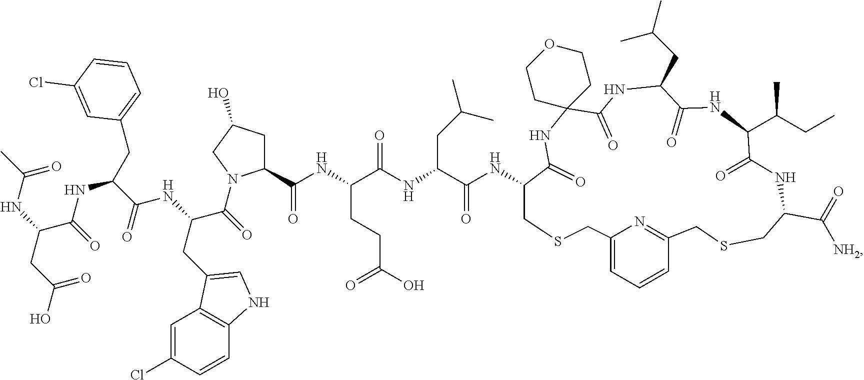 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2cccc(Cl)c2)NC(=O)[C@H](CC(=O)O)NC(C)=O)CSCc2cccc(n2)CSC[C@@H](C(N)=O)NC1=O]
compound Ac-Asp-Pcf-5Clw-Hyp-Glu-leu-[Cys(2Lut)-Thp-Leu-Ile-Cys]-NH₂ (GIP-210) of the 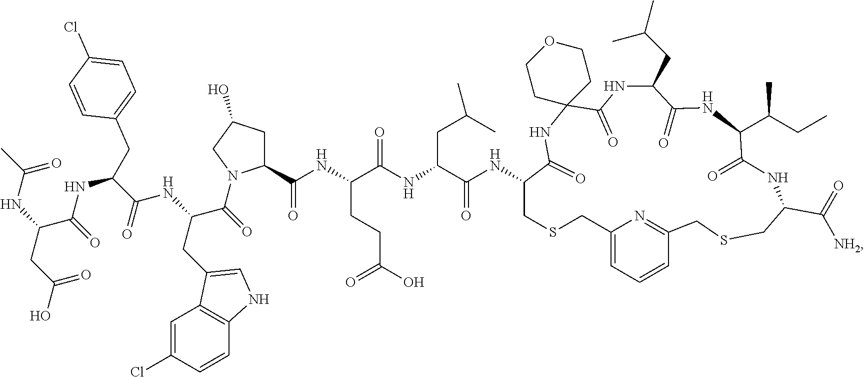 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2ccc(Cl)cc2)NC(=O)[C@H](CC(=O)O)NC(C)=O)CSCc2cccc(n2)CSC[C@@H](C(N)=O)NC1=O]
compound Ac-Asp-Eaa-5Clw-Hyp-Glu-leu-[Cys(2Lut)-Thp-Leu-Ile-Cys]-NH₂ (GIP-211) of the following formula 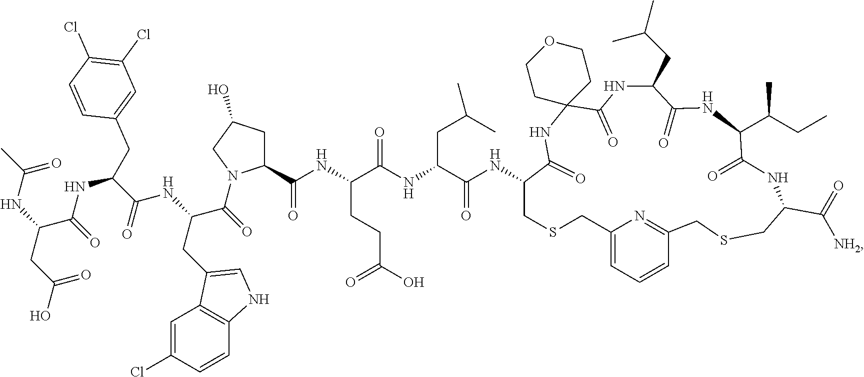 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2ccc(Cl)c(Cl)c2)NC(=O)[C@H](CC(=O)O)NC(C)=O)CSCc2cccc(n2)CSC[C@@H](C(N)=O)NC1=O]
compound Ac-Asp-Egm-5Clw-Hyp-Glu-leu-[Cys(2Lut)-Thp-Leu-Ile-Cys]-NH₂ (GIP-212) of the following formula 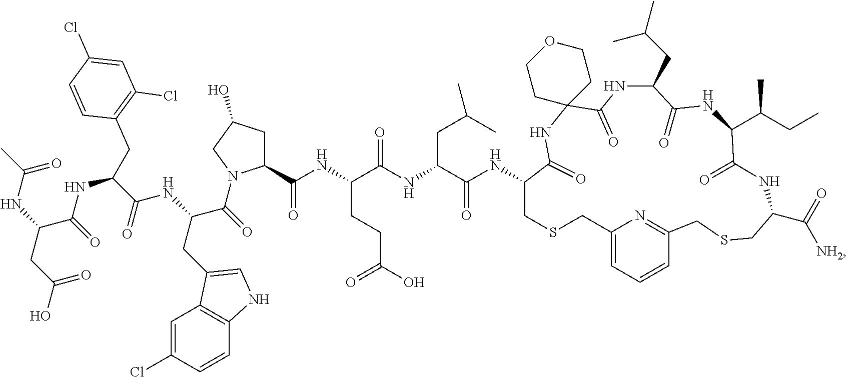 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2ccc(Cl)cc2Cl)NC(=O)[C@H](CC(=O)O)NC(C)=O)CSCc2cccc(n2)CSC[C@@H](C(N)=O)NC1=O]
compound Ac-Asp-Trp-Eaa-Hyp-Glu-leu-[Cys(2Lut)-Thp-Leu-Ile-Cys]-NH₂ (GIP-213) of the following formula 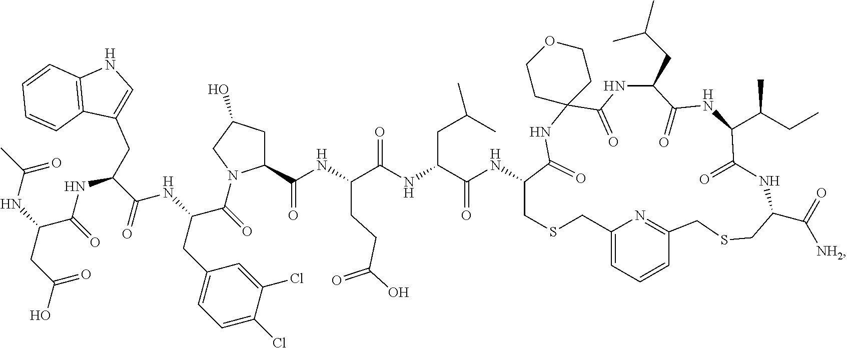 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2ccc(Cl)c(Cl)c2)NC(=O)[C@H](Cc2c[nH]c3ccccc23)NC(=O)[C@H](CC(=O)O)NC(C)=O)CSCc2cccc(n2)CSC[C@@H](C(N)=O)NC1=O]
compound HO-Glutar-Trp-5Clw-Hyp-Glu-leu-[Cys(2Lut)-Thp-Leu-Ile-Cys]-NH₂ (GIP-214) of the following formula 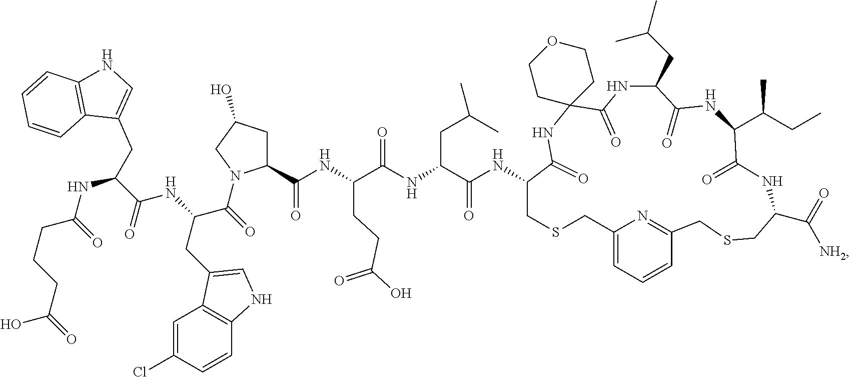 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)CCCC(=O)O)CSCc2cccc(n2)CSC[C@@H](C(N)=O)NC1=O]
compound Ac-Asp-Trp-5Clw-Dtc-Glu-leu-[Cys(2Lut)-Thp-Leu-Ile-Cys]-NH₂ (GIP-215) of the following formula 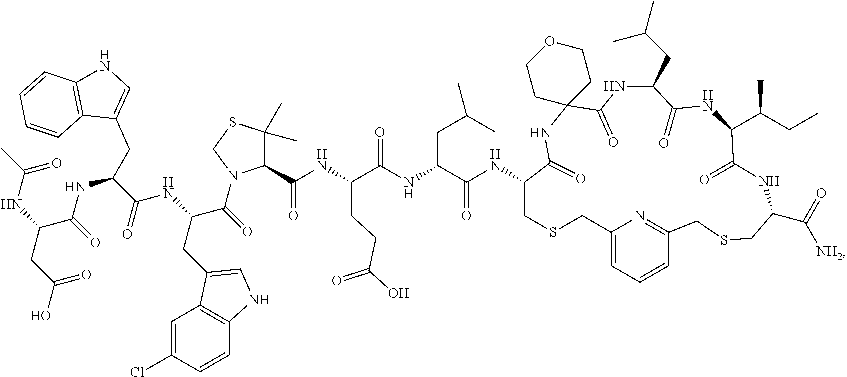 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@H]2N(C(=O)[C@H](Cc3c[nH]c4ccc(Cl)cc34)NC(=O)[C@H](Cc3c[nH]c4ccccc34)NC(=O)[C@H](CC(=O)O)NC(C)=O)CSC2(C)C)CSCc2cccc(n2)CSC[C@@H](C(N)=O)NC1=O]
compound Ac-Asp-Trp-5Clw-Oxa-Glu-leu-[Cys(2Lut)-Thp-Leu-Ile-Cys]-NH₂ (GIP-216) of the following formula 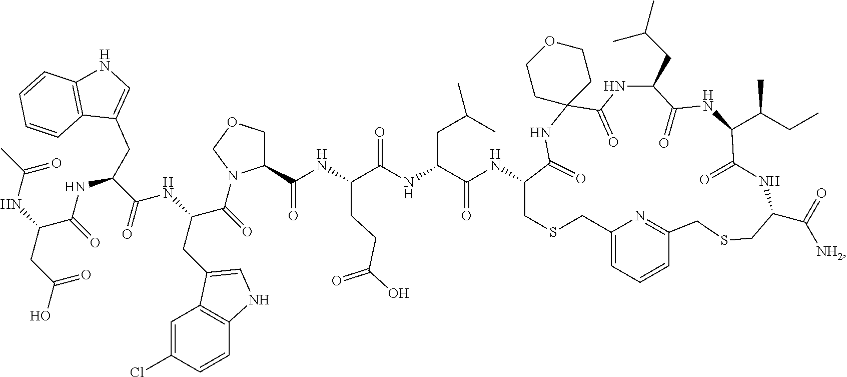 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2COCN2C(=O)[C@H](Cc2c[nH]c3ccc(Cl)cc23)NC(=O)[C@H](Cc2c[nH]c3ccccc23)NC(=O)[C@H](CC(=O)O)NC(C)=O)CSCc2cccc(n2)CSC[C@@H](C(N)=O)NC1=O]
compound Ac-Asp-Trp-5Clw-Hyp-Nme-Leu-[Cys(2Lut)-Thp-Leu-Ile-Cys]-NH₂ (GIP-217) of the following formula 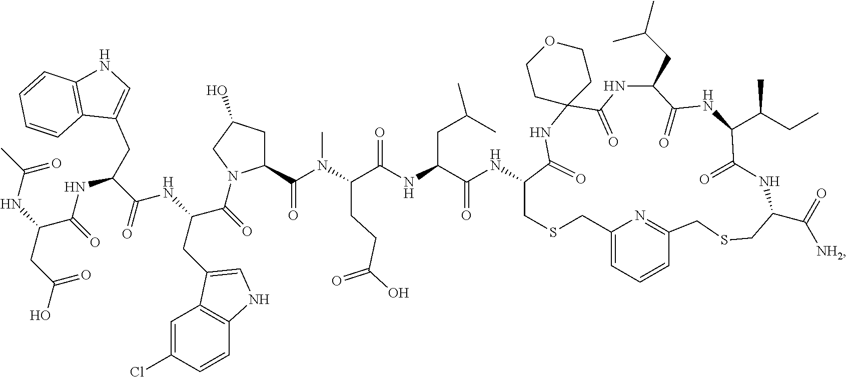 [SMILES: CC[C@H](C)[C@@H]1NC(=O)[C@H](CC(C)C)NC(=O)C2(CCOCC2)NC(=O)[C@@H](NC(=O)[C@H](CC(C)C)NC(=O)[C@H](CCC(=O)O)N(C)C(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CC(=O)O)NC(C)=O)CSCc2cccc(n2)CSC[C@@H](C(N)=O)NC1=O]
compound Ac-Asp-Trp-5Clw-Hyp-Glu-Ile-[Cys(2Lut)-Thp-Leu-Ile-Cys]-NH₂ (GIP-218) of the following formula 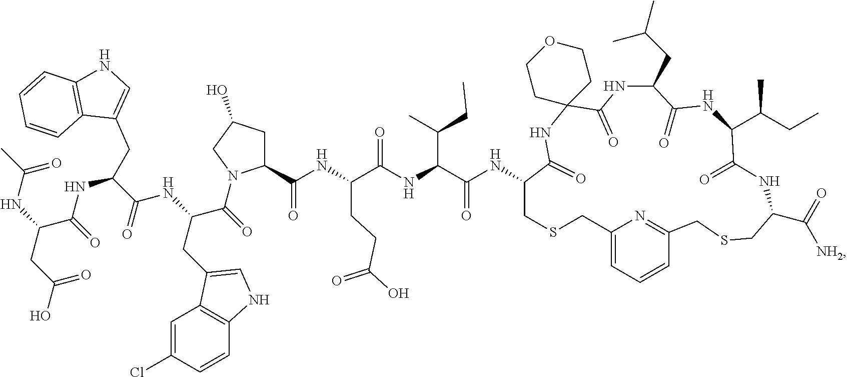 [SMILES: CC[C@H](C)[C@@H]1NC(=O)[C@H](CC(C)C)NC(=O)C2(CCOCC2)NC(=O)[C@@H](NC(=O)[C@@H](NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CC(=O)O)NC(C)=O)[C@@H](C)CC)CSCc2cccc(n2)CSC[C@@H](C(N)=O)NC1=O]
compound Ac-Asp-Trp-5Clw-Hyp-Glu-Val-[Cys(2Lut)-Thp-Leu-Ile-Cys]-NH₂ (GIP-219) of the following formula 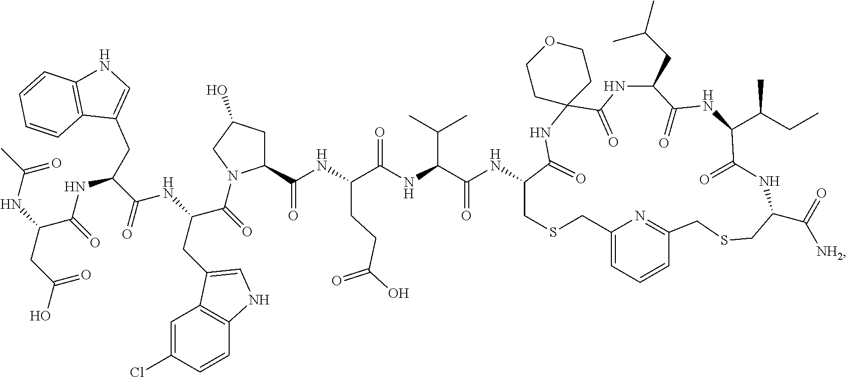 [SMILES: CC[C@H](C)[C@@H]1NC(=O)[C@H](CC(C)C)NC(=O)C2(CCOCC2)NC(=O)[C@@H](NC(=O)[C@@H](NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CC(=O)O)NC(C)=O)C(C)C)CSCc2cccc(n2)CSC[C@@H](C(N)=O)NC1=O]
compound Ac-Asp-Hyw-5Clw-Hyp-Glu-leu-[Cys(2Lut)-Thp-Leu-Ile-Cys]-NH₂ (GIP-220) of the following formula 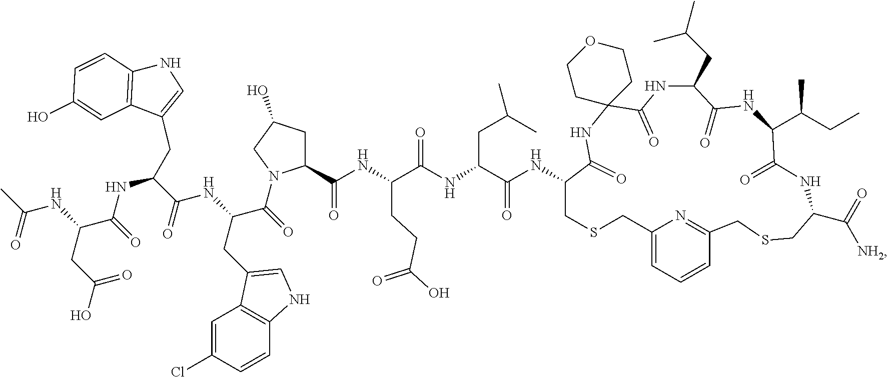 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3ccc(O)cc23)NC(=O)[C@H](CC(=O)O)NC(C)=O)CSCc2cccc(n2)CSC[C@@H](C(N)=O)NC1=O]
compound Ac-Asp-7Fw-5Clw-Hyp-Glu-leu-[Cys(2Lut)-Thp-Leu-Ile-Cys]-NH₂ (GIP-221) of the following formula 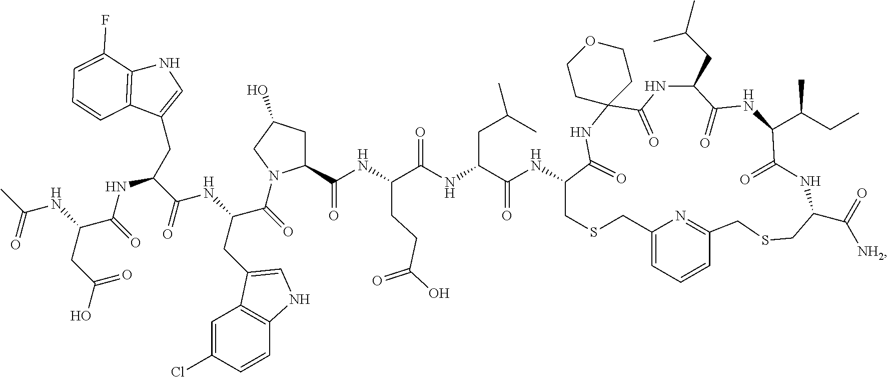 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3c(F)cccc23)NC(=O)[C@H](CC(=O)O)NC(C)=O)CSCc2cccc(n2)CSC[C@@H](C(N)=O)NC1=O]
compound Ac-Asp-Trp-5Clw-Eaz-Glu-leu-[Cys(2Lut)-Thp-Leu-Ile-Cys]-NH₂ (GIP-222) of the 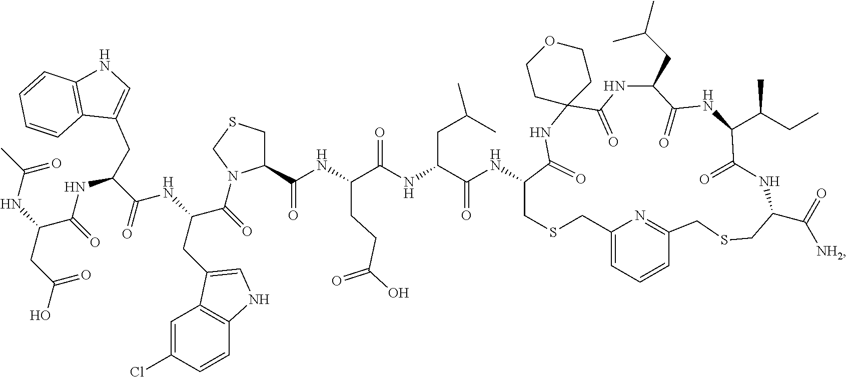 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2CSCN2C(=O)[C@H](Cc2c[nH]c3ccc(Cl)cc23)NC(=O)[C@H](Cc2c[nH]c3ccccc23)NC(=O)[C@H](CC(=O)O)NC(C)=O)CSCc2cccc(n2)CSC[C@@H](C(N)=O)NC1=O]
compound Ac-Asp-Trp-5Clw-Hyp-glu-Leu-[Cys(2Lut)-Thp-Leu-Ile-Cys]-NH₂ (GIP-223) of the following formula 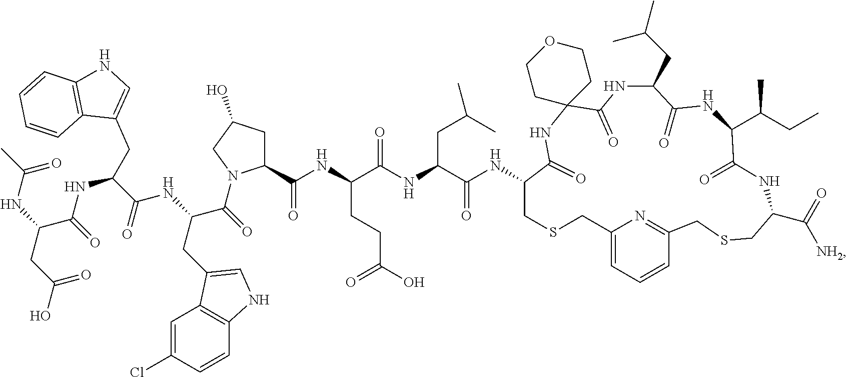 [SMILES: CC[C@H](C)[C@@H]1NC(=O)[C@H](CC(C)C)NC(=O)C2(CCOCC2)NC(=O)[C@@H](NC(=O)[C@H](CC(C)C)NC(=O)[C@@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CC(=O)O)NC(C)=O)CSCc2cccc(n2)CSC[C@@H](C(N)=O)NC1=O]
compound Ac-Asp-Phe-5Clw-Hyp-Glu-leu-[Cys(2Lut)-Thp-Leu-Ile-Cys]-NH₂ (GIP-224) of the following formula 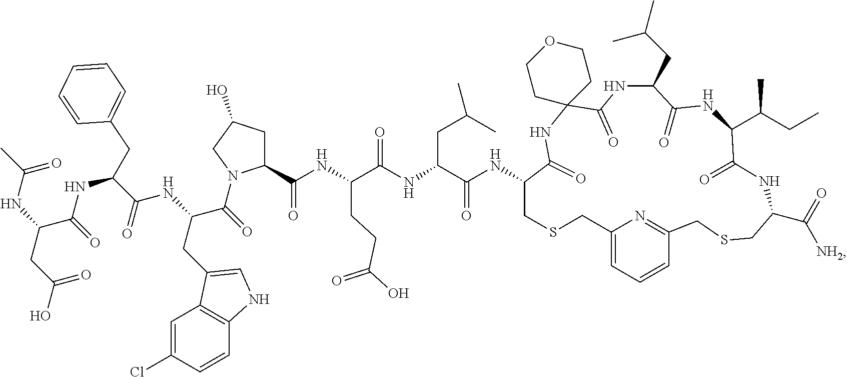 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2ccccc2)NC(=O)[C@H](CC(=O)O)NC(C)=O)CSCc2cccc(n2)CSC[C@@H](C(N)=O)NC1=O]
compound Ac-Asp-Egp-5Clw-Hyp-Glu-leu-[Cys(2Lut)-Thp-Leu-Ile-Cys]-NH₂ (GIP-226) of the following formula 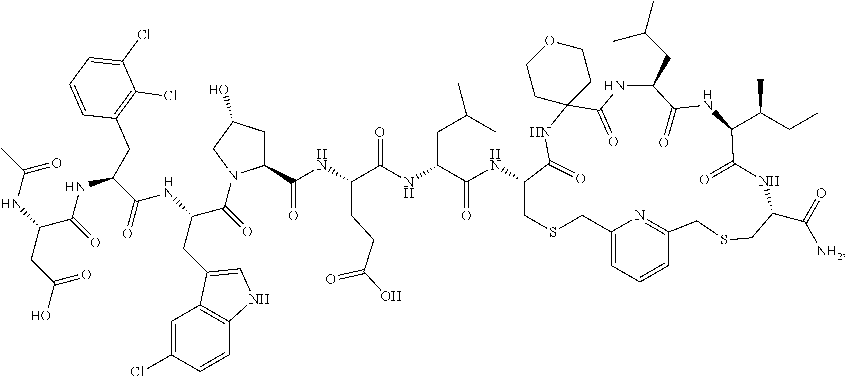 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2cccc(Cl)c2Cl)NC(=O)[C@H](CC(=O)O)NC(C)=O)CSCc2cccc(n2)CSC[C@@H](C(N)=O)NC1=O]
compound Ac-Asp-Trp-7Fw-Hyp-Glu-leu-[Cys(2Lut)-Thp-Leu-Ile-Cys]-NH₂ (GIP-227) of the 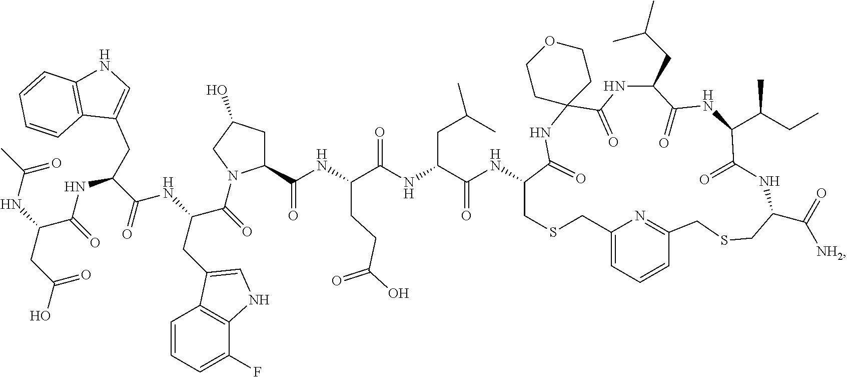 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3c(F)cccc23)NC(=O)[C@H](Cc2c[nH]c3ccccc23)NC(=O)[C@H](CC(=O)O)NC(C)=O)CSCc2cccc(n2)CSC[C@@H](C(N)=O)NC1=O]
compound Ac-Asp-Trp-5Clw-Hyp-Glu-leu-[Cys(2Lut)-Phe-Leu-Ile-Cys]-NH₂ (GIP-228) of the following formula 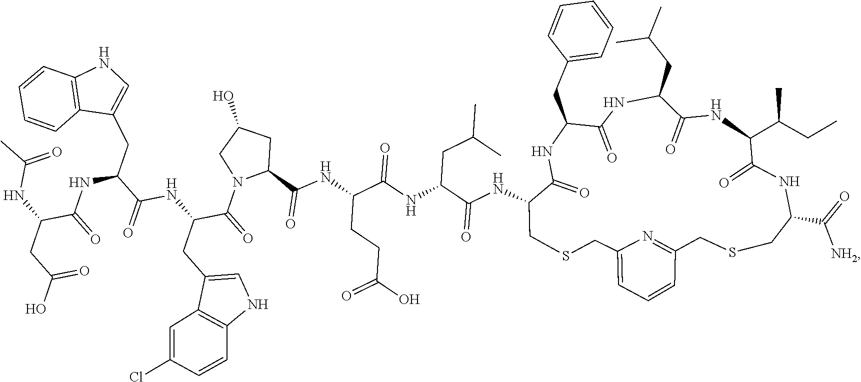 [SMILES: CC[C@H](C)[C@@H]1NC(=O)[C@H](CC(C)C)NC(=O)[C@H](Cc2ccccc2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CC(=O)O)NC(C)=O)CSCc2cccc(n2)CSC[C@@H](C(N)=O)NC1=O]
compound Ac-Asp-Trp-5Clw-Hyp-Glu-leu-[Cys(2Lut)-Thp-Ile-Ile-Cys]-NH₂ (GIP-229) of the following formula 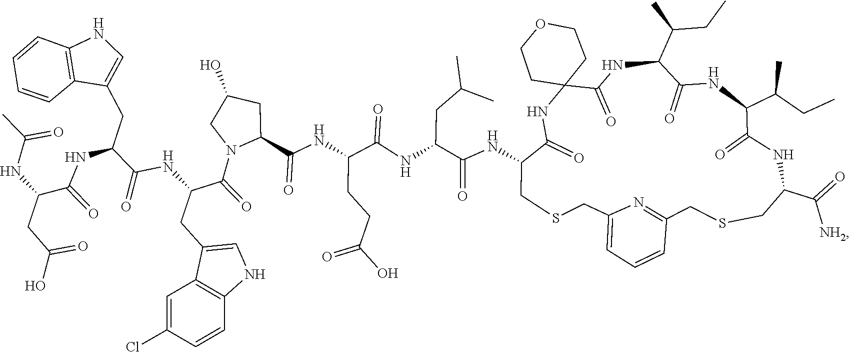 [SMILES: CC[C@H](C)[C@@H]1NC(=O)[C@H]([C@@H](C)CC)NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CC(=O)O)NC(C)=O)CSCc2cccc(n2)CSC[C@@H](C(N)=O)NC1=O]
compound Ac-Asp-Trp-5Clw-Hyp-Glu-leu-[Cys(2Lut)-Thp-Arg-Ile-Cys]-NH₂ (GIP-230) of the following formula 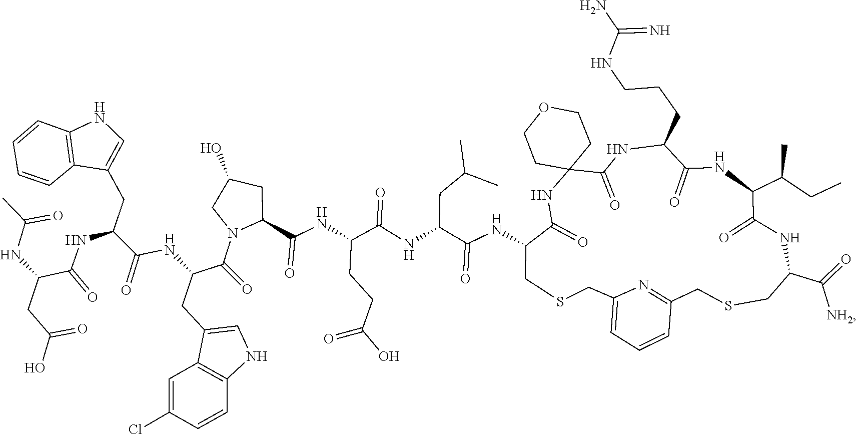 [SMILES: CC[C@H](C)[C@@H]1NC(=O)[C@H](CCCNC(=N)N)NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CC(=O)O)NC(C)=O)CSCc2cccc(n2)CSC[C@@H](C(N)=O)NC1=O]
compound Ac-Asp-Trp-5Clw-Hyp-Glu-leu-[Cys(2Lut)-Thp-Glu-Ile-Cys]-NH₂ (GIP-231) of the following formula 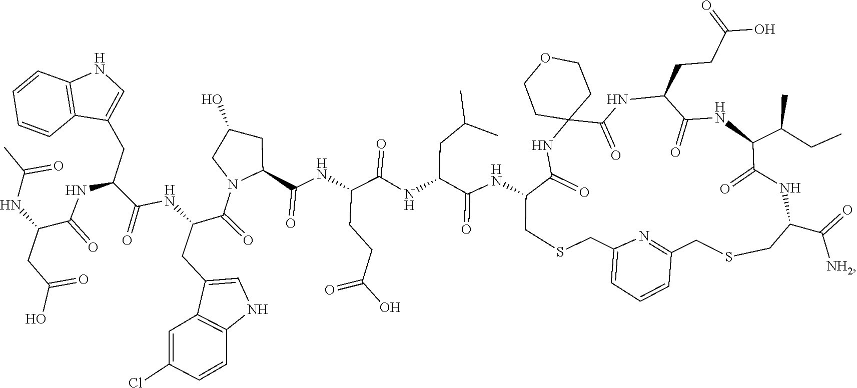 [SMILES: CC[C@H](C)[C@@H]1NC(=O)[C@H](CCC(=O)O)NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CC(=O)O)NC(C)=O)CSCc2cccc(n2)CSC[C@@H](C(N)=O)NC1=O]
compound Ac-Asp-Trp-5Clw-Hyp-Glu-leu-[Cys(2Lut)-Thp-Phe-Ile-Cys]-NH₂ (GIP-232) of the following formula 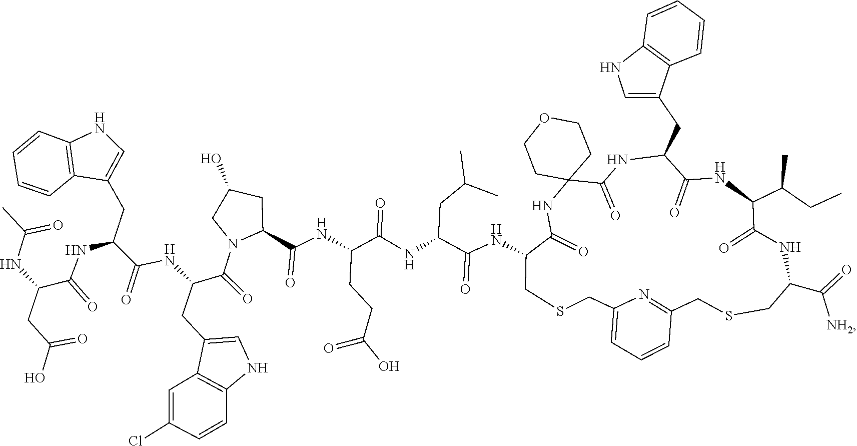 [SMILES: CC[C@H](C)[C@@H]1NC(=O)[C@H](Cc2c[nH]c3ccccc23)NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CC(=O)O)NC(C)=O)CSCc2cccc(n2)CSC[C@@H](C(N)=O)NC1=O]
compound Ac-Asp-Trp-5Clw-Hyp-Glu-leu-[Cys(2Lut)-Thp-Trp-Ile-Cys]-NH₂ (GIP-233) of the following formula 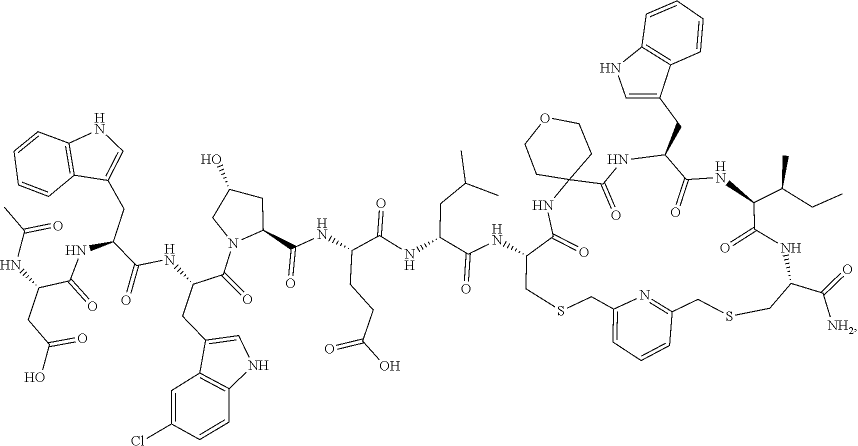 [SMILES: CC[C@H](C)[C@@H]1NC(=O)[C@H](Cc2c[nH]c3ccccc23)NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CC(=O)O)NC(C)=O)CSCc2cccc(n2)CSC[C@@H](C(N)=O)NC1=O]
compound Ac-Asp-Trp-5Clw-Hyp-Glu-leu-[Cys(2Lut)-Trp-Leu-Ile-Cys]-NH₂ (GIP-234) of the 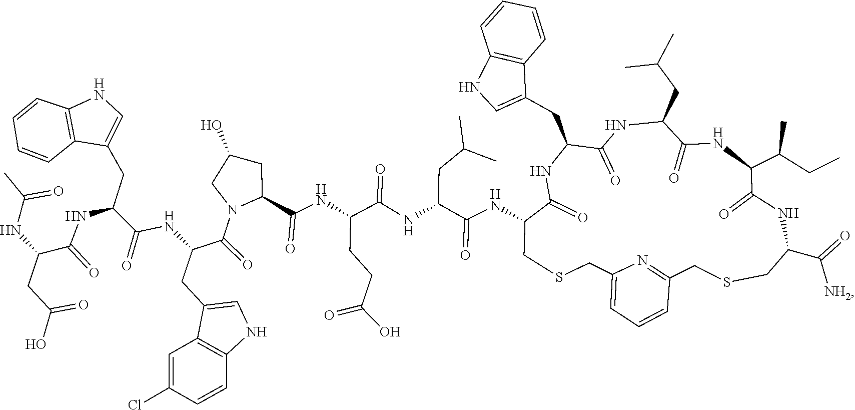 [SMILES: CC[C@H](C)[C@@H]1NC(=O)[C@H](CC(C)C)NC(=O)[C@H](Cc2c[nH]c3ccccc23)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CC(=O)O)NC(C)=O)CSCc2cccc(n2)CSC[C@@H](C(N)=O)NC1=O]
compound Ac-Asp-Trp-5Clw-Hyp-Glu-leu-[Cys(2Lut)-Glu-Leu-Ile-Cys]-NH₂ (GIP-235) of the following formula 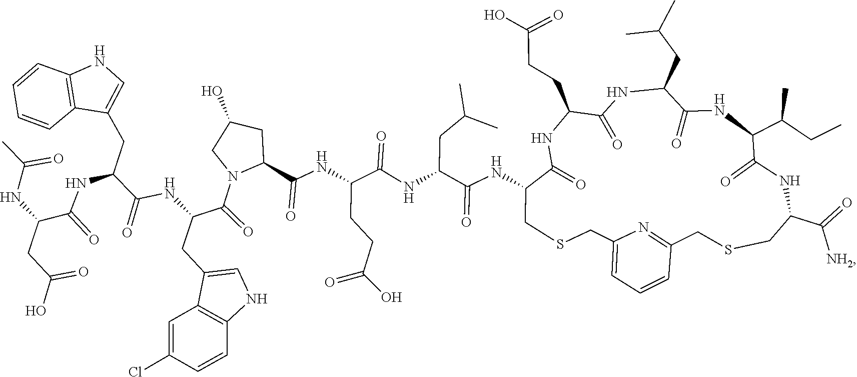 [SMILES: CC[C@H](C)[C@@H]1NC(=O)[C@H](CC(C)C)NC(=O)[C@H](CCC(=O)O)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CC(=O)O)NC(C)=O)CSCc2cccc(n2)CSC[C@@H](C(N)=O)NC1=O]
compound Ac-Asp-Trp-5Clw-Hyp-Glu-leu-[Cys(2Lut)-Thp-Ser-Ile-Cys]-NH₂ (GIP-236) of the following formula 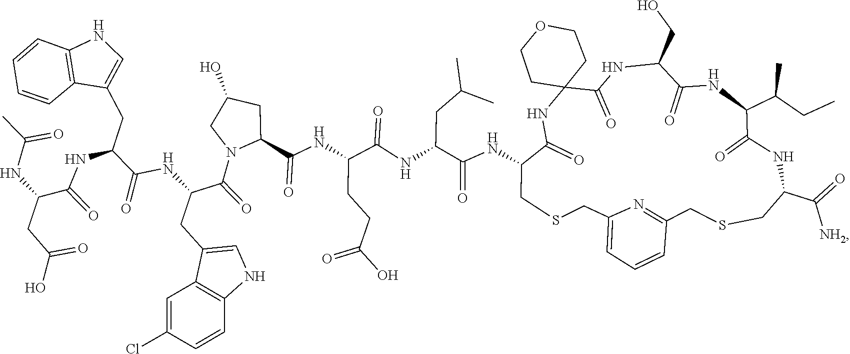 [SMILES: CC[C@H](C)[C@@H]1NC(=O)[C@H](CO)NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CC(=O)O)NC(C)=O)CSCc2cccc(n2)CSC[C@@H](C(N)=O)NC1=O]
compound Ac-Asp-Trp-5Clw-Hyp-Glu-leu-[Cys(2Lut)-Thp-Leu-Leu-Cys]-NH₂ (GIP-237) of the following formula 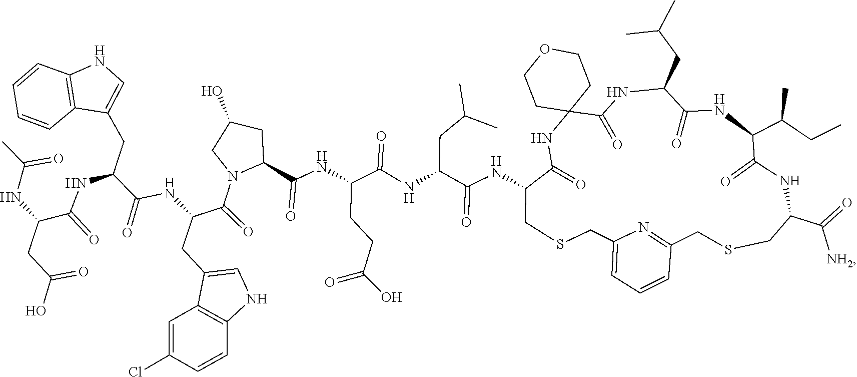 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CC(=O)O)NC(C)=O)CSCc2cccc(n2)CSC[C@@H](C(N)=O)NC1=O]
compound Ac-Asp-Trp-5Clw-Hyp-Glu-leu-[Cys(2Lut)-Thp-Leu-Tle-Cys]-NH₂ (GIP-238) of the 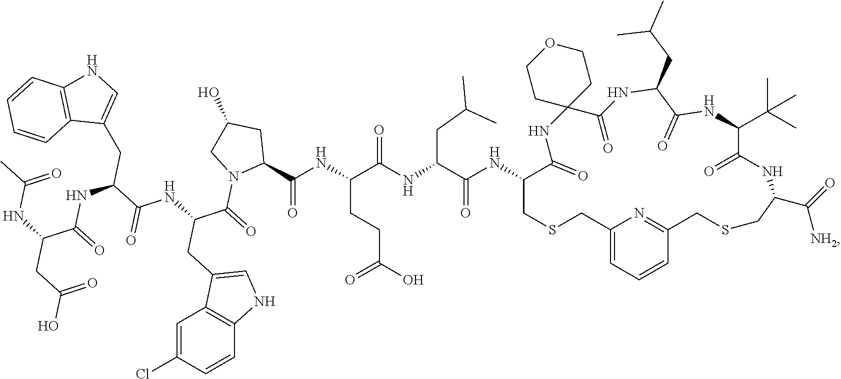 [SMILES: CC(=O)N[C@@H](CC(=O)O)C(=O)N[C@@H](Cc1c[nH]c2ccccc12)C(=O)N[C@@H](Cc1c[nH]c2ccc(Cl)cc12)C(=O)N1C[C@H](O)C[C@H]1C(=O)N[C@@H](CCC(=O)O)C(=O)N[C@H](CC(C)C)C(=O)N[C@H]1CSCc2cccc(n2)CSC[C@@H](C(N)=O)NC(=O)[C@H](C(C)(C)C)NC(=O)[C@H](CC(C)C)NC(=O)C2(CCOCC2)NC1=O]
compound Ac-Asp-Trp-5Clw-Tap-Glu-leu-[Cys(2Lut)-Thp-Leu-Ile-Cys]-NH₂ (GIP-239) of the following formula 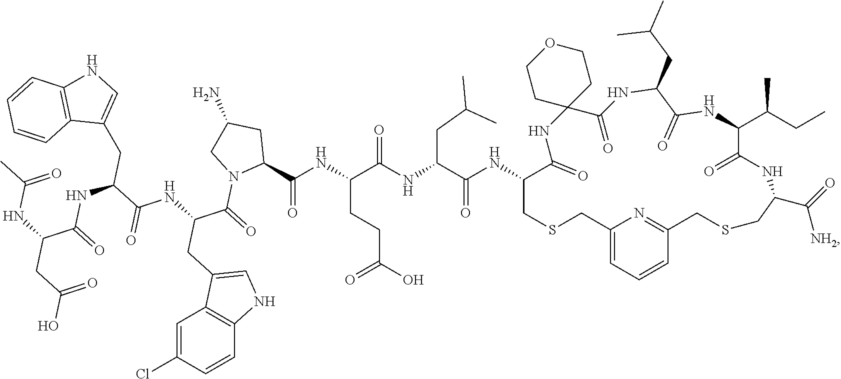 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](N)CN2C(=O)[C@H](Cc2c[nH]c3ccc(Cl)cc23)NC(=O)[C@H](Cc2c[nH]c3ccccc23)NC(=O)[C@H](CC(=O)O)NC(C)=O)CSCc2cccc(n2)CSC[C@@H](C(N)=O)NC1=O]
compound Ac-Tyr-Asp-Trp-Trp-Pro-Glu-Leu-[Cys(3MeBn)-Ala-Leu-Ile-Cys]-NH₂ (GIP-240) of the following formula 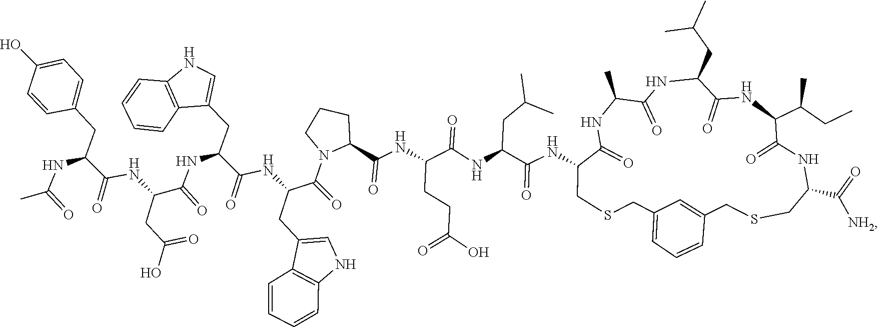 [SMILES: CC[C@H](C)[C@@H]1NC(=O)[C@H](CC(C)C)NC(=O)[C@H](C)NC(=O)[C@@H](NC(=O)[C@H](CC(C)C)NC(=O)[C@H](CCC(=O)O)NC(=O)[C@@H]2CCCN2C(=O)[C@H](Cc2c[nH]c3ccccc23)NC(=O)[C@H](Cc2c[nH]c3ccccc23)NC(=O)[C@H](CC(=O)O)NC(=O)[C@H](Cc2ccc(O)cc2)NC(C)=O)CSCc2cccc(c2)CSC[C@@H](C(N)=O)NC1=O]
compound H-Met-Tyr-Asp-Trp-Trp-Pro-Glu-Leu-[Cys(3MeBn)-Gln-Leu-Ile-Cys]-NH₂ (GIP-241) of the following formula 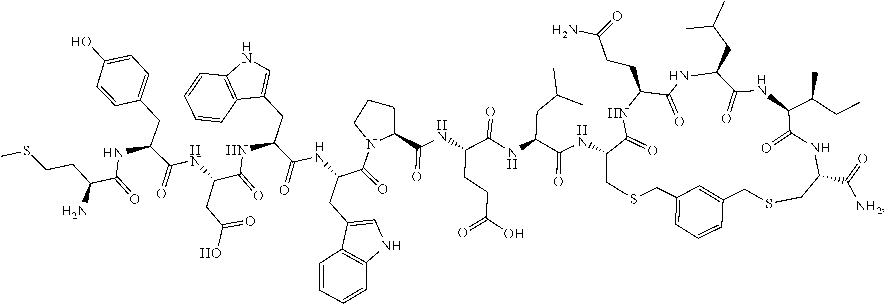 [SMILES: CC[C@H](C)[C@@H]1NC(=O)[C@H](CC(C)C)NC(=O)[C@H](CCC(N)=O)NC(=O)[C@@H](NC(=O)[C@H](CC(C)C)NC(=O)[C@H](CCC(=O)O)NC(=O)[C@@H]2CCCN2C(=O)[C@H](Cc2c[nH]c3ccccc23)NC(=O)[C@H](Cc2c[nH]c3ccccc23)NC(=O)[C@H](CC(=O)O)NC(=O)[C@H](Cc2ccc(O)cc2)NC(=O)[C@@H](N)CCSC)CSCc2cccc(c2)CSC[C@@H](C(N)=O)NC1=O]
compound H-Met-Tyr-Asp-Trp-Trp-Pro-Glu-Leu-[Cys(3MeBn)-Thr-Leu-Ile-Cys]-NH₂ (GIP-242) of the following formula 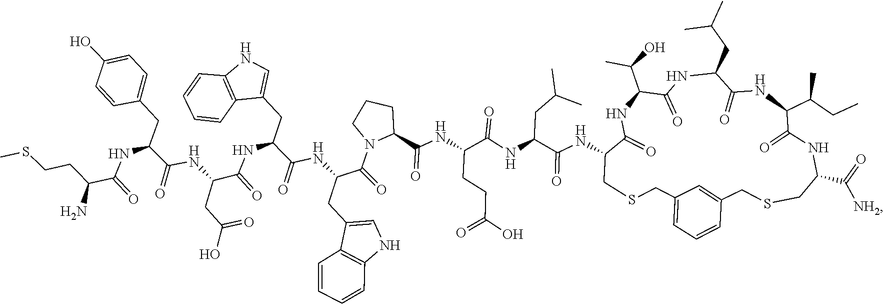 [SMILES: CC[C@H](C)[C@@H]1NC(=O)[C@H](CC(C)C)NC(=O)[C@H]([C@@H](C)O)NC(=O)[C@@H](NC(=O)[C@H](CC(C)C)NC(=O)[C@H](CCC(=O)O)NC(=O)[C@@H]2CCCN2C(=O)[C@H](Cc2c[nH]c3ccccc23)NC(=O)[C@H](Cc2c[nH]c3ccccc23)NC(=O)[C@H](CC(=O)O)NC(=O)[C@H](Cc2ccc(O)cc2)NC(=O)[C@@H](N)CCSC)CSCc2cccc(c2)CSC[C@@H](C(N)=O)NC1=O]
compound H-Met-Tyr-Asp-Trp-Trp-Pro-Glu-Leu-[Cys(3MeBn)-Lys-Leu-Ile-Cys]-NH₂ (GIP-243) of the following formula 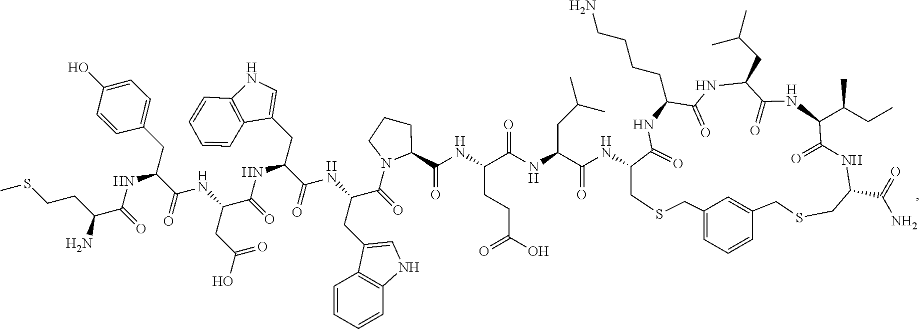 [SMILES: CC[C@H](C)[C@@H]1NC(=O)[C@H](CC(C)C)NC(=O)[C@H](CCCCN)NC(=O)[C@@H](NC(=O)[C@H](CC(C)C)NC(=O)[C@H](CCC(=O)O)NC(=O)[C@@H]2CCCN2C(=O)[C@H](Cc2c[nH]c3ccccc23)NC(=O)[C@H](Cc2c[nH]c3ccccc23)NC(=O)[C@H](CC(=O)O)NC(=O)[C@H](Cc2ccc(O)cc2)NC(=O)[C@@H](N)CCSC)CSCc2cccc(c2)CSC[C@@H](C(N)=O)NC1=O]
compound Ac-Asp-Trp-Trp-Pro-Glu-Leu-[Cys(3MeBn)-Ala-Leu-Ile-Cys]-NH₂ (GIP-244) of the following formula 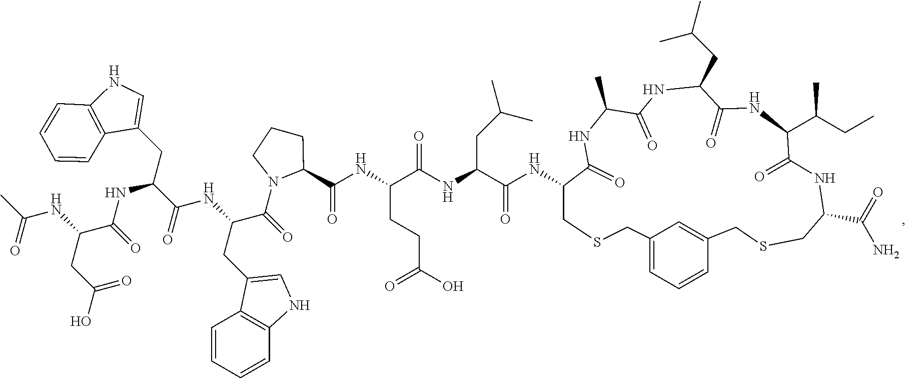 [SMILES: CC[C@H](C)[C@@H]1NC(=O)[C@H](CC(C)C)NC(=O)[C@H](C)NC(=O)[C@@H](NC(=O)[C@H](CC(C)C)NC(=O)[C@H](CCC(=O)O)NC(=O)[C@@H]2CCCN2C(=O)[C@H](Cc2c[nH]c3ccccc23)NC(=O)[C@H](Cc2c[nH]c3ccccc23)NC(=O)[C@H](CC(=O)O)NC(C)=O)CSCc2cccc(c2)CSC[C@@H](C(N)=O)NC1=O]
compound H-Met-Tyr-Asp-Trp-Trp-Pro-glu-Leu-[Cys(3MeBn)-Ala-Leu-Ile-Cys]-NH₂ (GIP-245) of the following formula 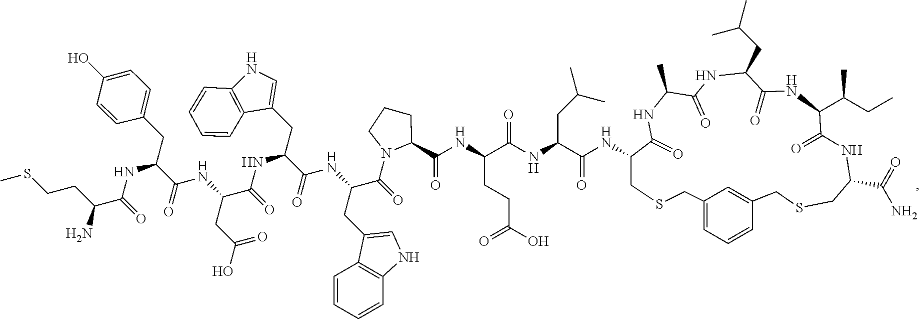 [SMILES: CC[C@H](C)[C@@H]1NC(=O)[C@H](CC(C)C)NC(=O)[C@H](C)NC(=O)[C@@H](NC(=O)[C@H](CC(C)C)NC(=O)[C@@H](CCC(=O)O)NC(=O)[C@@H]2CCCN2C(=O)[C@H](Cc2c[nH]c3ccccc23)NC(=O)[C@H](Cc2c[nH]c3ccccc23)NC(=O)[C@H](CC(=O)O)NC(=O)[C@H](Cc2ccc(O)cc2)NC(=O)[C@@H](N)CCSC)CSCc2cccc(c2)CSC[C@@H](C(N)=O)NC1=O]
compound H-Met-tyr-Asp-Trp-Trp-Pro-Glu-Leu-[Cys(3MeBn)-Ala-Leu-Ile-Cys]-NH₂ (GIP-246) of the following formula 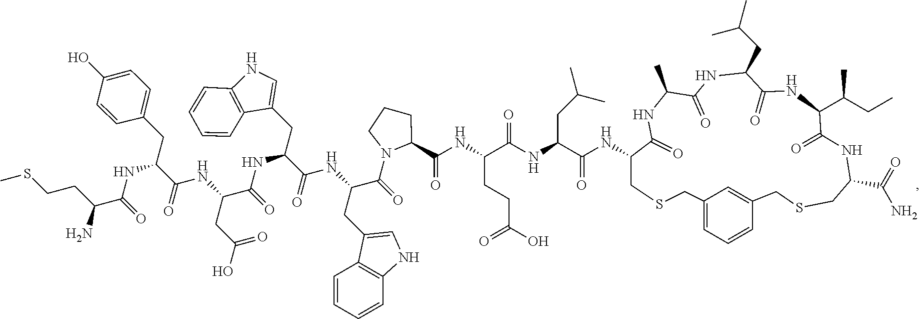 [SMILES: CC[C@H](C)[C@@H]1NC(=O)[C@H](CC(C)C)NC(=O)[C@H](C)NC(=O)[C@@H](NC(=O)[C@H](CC(C)C)NC(=O)[C@H](CCC(=O)O)NC(=O)[C@@H]2CCCN2C(=O)[C@H](Cc2c[nH]c3ccccc23)NC(=O)[C@H](Cc2c[nH]c3ccccc23)NC(=O)[C@H](CC(=O)O)NC(=O)[C@@H](Cc2ccc(O)cc2)NC(=O)[C@@H](N)CCSC)CSCc2cccc(c2)CSC[C@@H](C(N)=O)NC1=O]
compound H-Met-Tyr-Asp-Trp-Trp-Pro-Glu-leu-[Cys(3MeBn)-Ala-Leu-Ile-Cys]-NH₂ (GIP-247) of the following formula 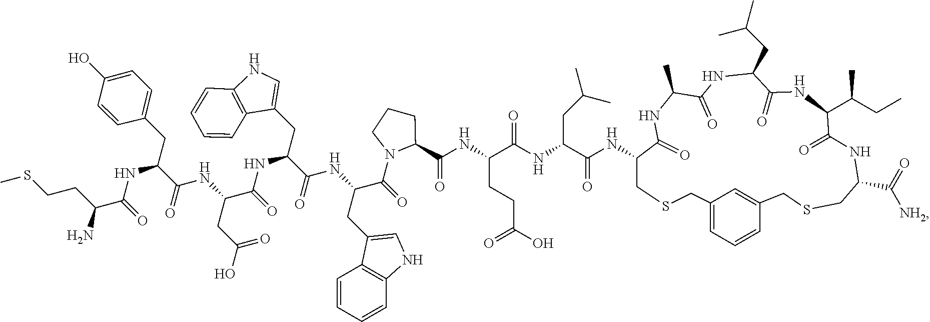
compound H-Met-Tyr-Asp-Trp-Trp-Pro-Glu-Leu-[Cys(3MeBn)-ala-Leu-Ile-Cys]-NH₂ (GIP-248) of the following formula 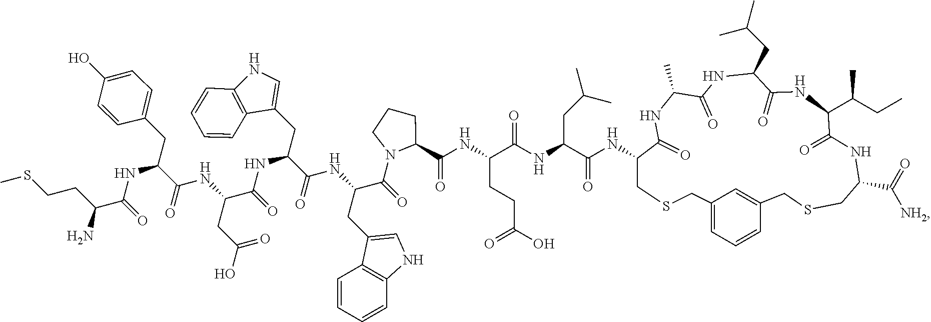 [SMILES: CC[C@H](C)[C@@H]1NC(=O)[C@H](CC(C)C)NC(=O)[C@@H](C)NC(=O)[C@@H](NC(=O)[C@H](CC(C)C)NC(=O)[C@H](CCC(=O)O)NC(=O)[C@@H]2CCCN2C(=O)[C@H](Cc2c[nH]c3ccccc23)NC(=O)[C@H](Cc2c[nH]c3ccccc23)NC(=O)[C@H](CC(=O)O)NC(=O)[C@H](Cc2ccc(O)cc2)NC(=O)[C@@H](N)CCSC)CSCc2cccc(c2)CSC[C@@H](C(N)=O)NC1=O]
compound H-Met-Ala-Asp-Trp-Trp-Pro-Glu-Leu-[Cys(3MeBn)-Ala-Leu-Ile-Cys]-NH₂ (GIP-249) of the following formula 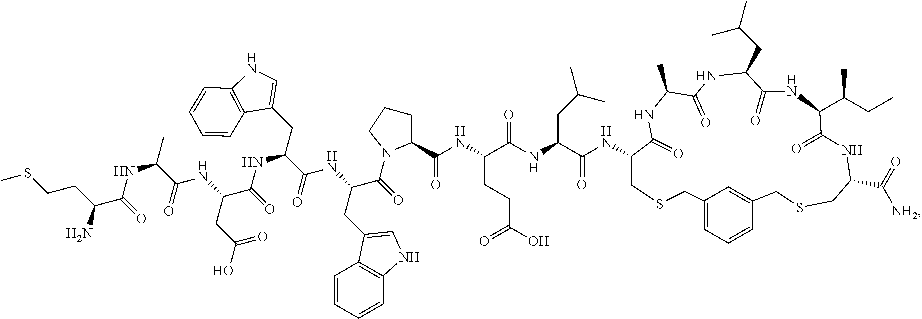 [SMILES: CC[C@H](C)[C@@H]1NC(=O)[C@H](CC(C)C)NC(=O)[C@H](C)NC(=O)[C@@H](NC(=O)[C@H](CC(C)C)NC(=O)[C@H](CCC(=O)O)NC(=O)[C@@H]2CCCN2C(=O)[C@H](Cc2c[nH]c3ccccc23)NC(=O)[C@H](Cc2c[nH]c3ccccc23)NC(=O)[C@H](CC(=O)O)NC(=O)[C@H](C)NC(=O)[C@@H](N)CCSC)CSCc2cccc(c2)CSC[C@@H](C(N)=O)NC1=O]
compound Ac-Asp-Trp-Trp-Pro-Glu-Leu-[Cys(3MeBn)-ala-Leu-Ile-AET] (GIP-250) of the following formula 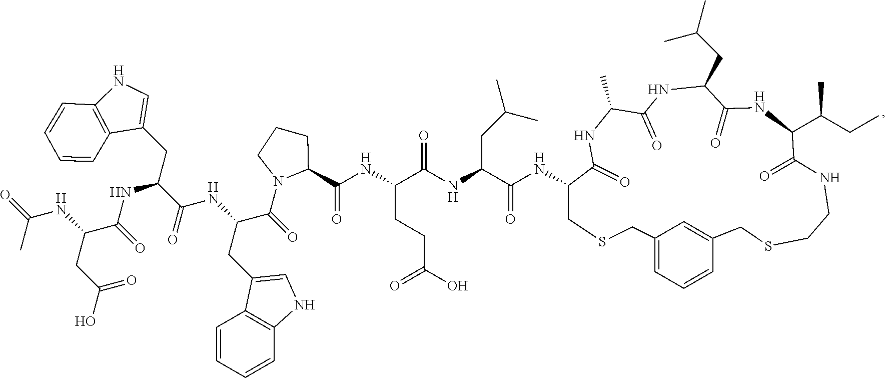 [SMILES: CC[C@H](C)[C@@H]1NC(=O)[C@H](CC(C)C)NC(=O)[C@@H](C)NC(=O)[C@@H](NC(=O)[C@H](CC(C)C)NC(=O)[C@H](CCC(=O)O)NC(=O)[C@@H]2CCCN2C(=O)[C@H](Cc2c[nH]c3ccccc23)NC(=O)[C@H](Cc2c[nH]c3ccccc23)NC(=O)[C@H](CC(=O)O)NC(C)=O)CSCc2cccc(c2)CSCCNC1=O]
compound Ac-Asp-Trp-Trp-Pro-Glu-Leu-[Cys(3MeBn)-Aib-Leu-Ile-AET] (GIP-251) of the following formula 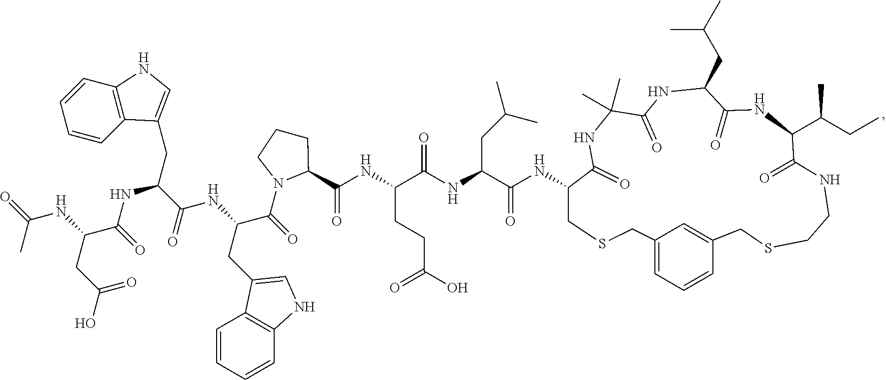 [SMILES: CC[C@H](C)[C@@H]1NC(=O)[C@H](CC(C)C)NC(=O)C(C)(C)NC(=O)[C@@H](NC(=O)[C@H](CC(C)C)NC(=O)[C@H](CCC(=O)O)NC(=O)[C@@H]2CCCN2C(=O)[C@H](Cc2c[nH]c3ccccc23)NC(=O)[C@H](Cc2c[nH]c3ccccc23)NC(=O)[C@H](CC(=O)O)NC(C)=O)CSCc2cccc(c2)CSCCNC1=O]
compound Ac-Asp-Trp-1Ni-Oic-Glu-Leu-[Cys(3MeBn)-Aib-Leu-Ile-Cys]-NH₂ (GIP-252) of the following formula 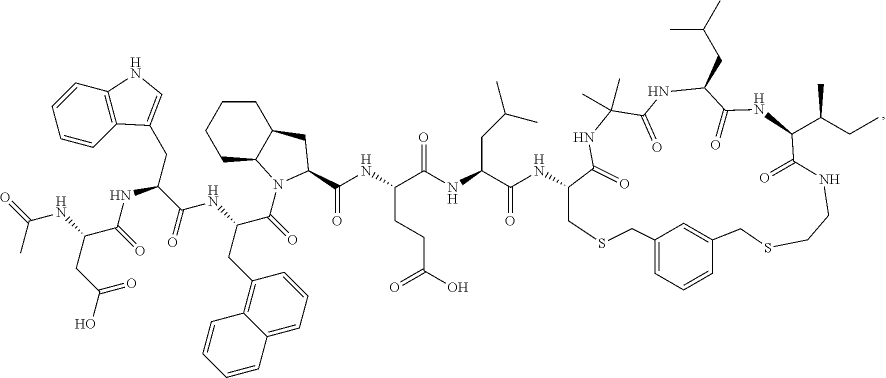 [SMILES: CC[C@H](C)[C@@H]1NC(=O)[C@H](CC(C)C)NC(=O)C(C)(C)NC(=O)[C@@H](NC(=O)[C@H](CC(C)C)NC(=O)[C@H](CCC(=O)O)NC(=O)[C@@H]2C[C@@H]3CCCC[C@@H]3N2C(=O)[C@H](Cc2cccc3ccccc23)NC(=O)[C@H](Cc2c[nH]c3ccccc23)NC(=O)[C@H](CC(=O)O)NC(C)=O)CSCc2cccc(c2)CSCCNC1=O]
compound Ac-Asp-Trp-1Ni-Pip-Glu-Leu-[Cys(3MeBn)-Aib-Leu-Ile-Cys]-NH₂ (GIP-253) of the following formula 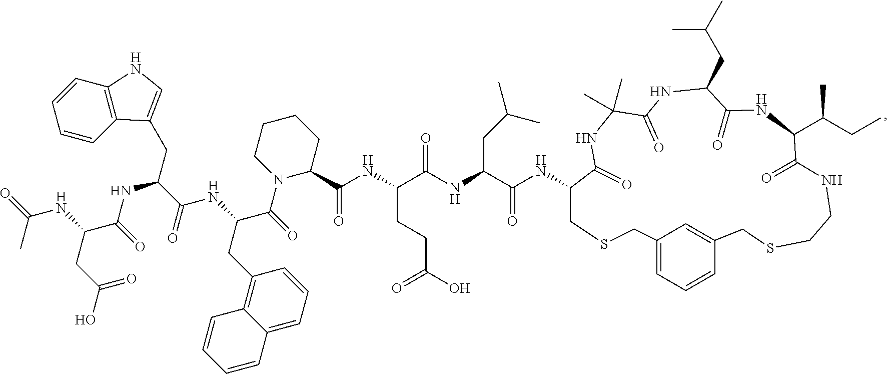 [SMILES: CC[C@H](C)[C@@H]1NC(=O)[C@H](CC(C)C)NC(=O)C(C)(C)NC(=O)[C@@H](NC(=O)[C@H](CC(C)C)NC(=O)[C@H](CCC(=O)O)NC(=O)[C@@H]2CCCCN2C(=O)[C@H](Cc2cccc3ccccc23)NC(=O)[C@H](Cc2c[nH]c3ccccc23)NC(=O)[C@H](CC(=O)O)NC(C)=O)CSCc2cccc(c2)CSCCNC1=O]
compound H-Met-Tyr-Asp-Trp-Trp-Pro-Glu-Leu-[Cys(3MeBn)-Ala-Leu-Ile-Cys]-NH₂ (GIP-254) of the following formula 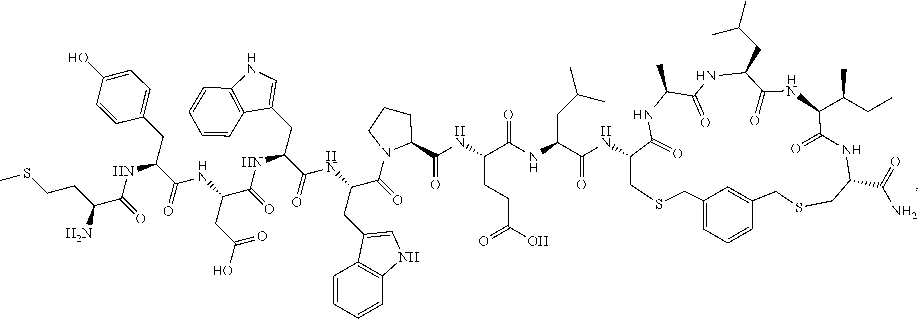 [SMILES: CC[C@H](C)[C@@H]1NC(=O)[C@H](CC(C)C)NC(=O)[C@H](C)NC(=O)[C@@H](NC(=O)[C@H](CC(C)C)NC(=O)[C@H](CCC(=O)O)NC(=O)[C@@H]2CCCN2C(=O)[C@H](Cc2c[nH]c3ccccc23)NC(=O)[C@H](Cc2c[nH]c3ccccc23)NC(=O)[C@H](CC(=O)O)NC(=O)[C@H](Cc2ccc(O)cc2)NC(=O)[C@@H](N)CCSC)CSCc2cccc(c2)CSC[C@@H](C(N)=O)NC1=O]
compound HO-Succinyl-Trp-Trp-Pro-Glu-Leu-[Cys(3MeBn)-Ala-Leu-Ile-Cys]-NH₂ (GIP-255) of the following formula 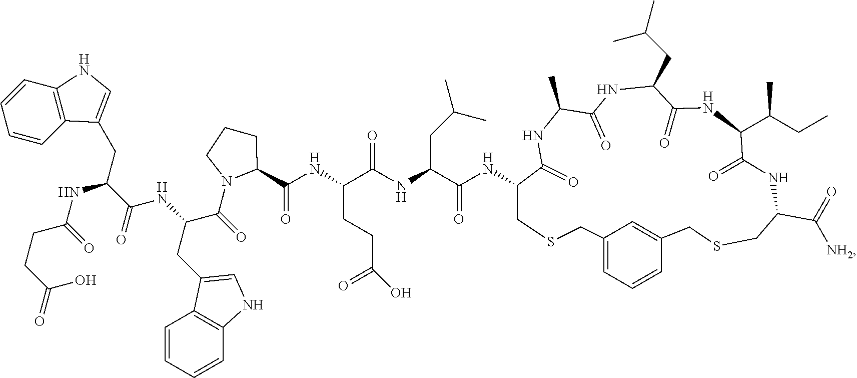 [SMILES: CC[C@H](C)[C@@H]1NC(=O)[C@H](CC(C)C)NC(=O)[C@H](C)NC(=O)[C@@H](NC(=O)[C@H](CC(C)C)NC(=O)[C@H](CCC(=O)O)NC(=O)[C@@H]2CCCN2C(=O)[C@H](Cc2c[nH]c3ccccc23)NC(=O)[C@H](Cc2c[nH]c3ccccc23)NC(=O)CCC(=O)O)CSCc2cccc(c2)CSC[C@@H](C(N)=O)NC1=O]
compound HO-Succinyl-Trp-Trp-Pro-Glu-Leu-[Cys(3MeBn)-Ala-Leu-Ile-AET] (GIP-256) of the following formula 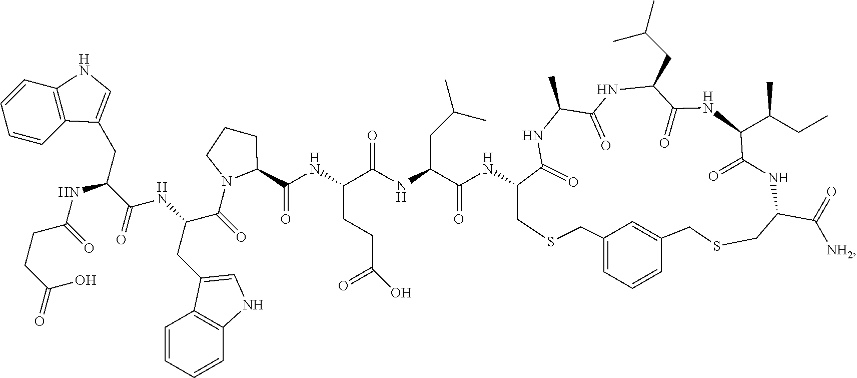 [SMILES: CC[C@H](C)[C@@H]1NC(=O)[C@H](CC(C)C)NC(=O)[C@H](C)NC(=O)[C@@H](NC(=O)[C@H](CC(C)C)NC(=O)[C@H](CCC(=O)O)NC(=O)[C@@H]2CCCN2C(=O)[C@H](Cc2c[nH]c3ccccc23)NC(=O)[C@H](Cc2c[nH]c3ccccc23)NC(=O)CCC(=O)O)CSCc2cccc(c2)CSC[C@@H](C(N)=O)NC1=O]
compound HO-Succinyl-Trp-Trp-Pro-Glu-Leu-[Cys(2Lut)-Ala-Leu-Ile-Cys]-NH₂ (GIP-257) of the following formula 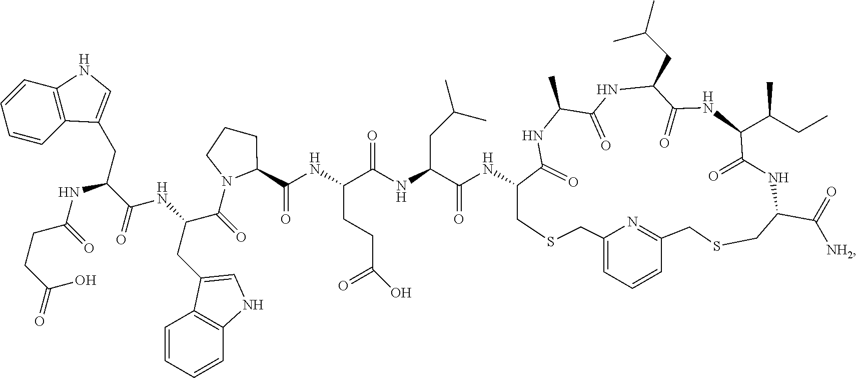 [SMILES: CC[C@H](C)[C@@H]1NC(=O)[C@H](CC(C)C)NC(=O)[C@H](C)NC(=O)[C@@H](NC(=O)[C@H](CC(C)C)NC(=O)[C@H](CCC(=O)O)NC(=O)[C@@H]2CCCN2C(=O)[C@H](Cc2c[nH]c3ccccc23)NC(=O)[C@H](Cc2c[nH]c3ccccc23)NC(=O)CCC(=O)O)CSCc2cccc(n2)CSC[C@@H](C(N)=O)NC1=O]
compound HO-Succinyl-Trp-Trp-Pro-Glu-leu-[Cys(3MeBn)-Aib-Leu-Ile-Cys]-NH₂ (GIP-258) of the following formula 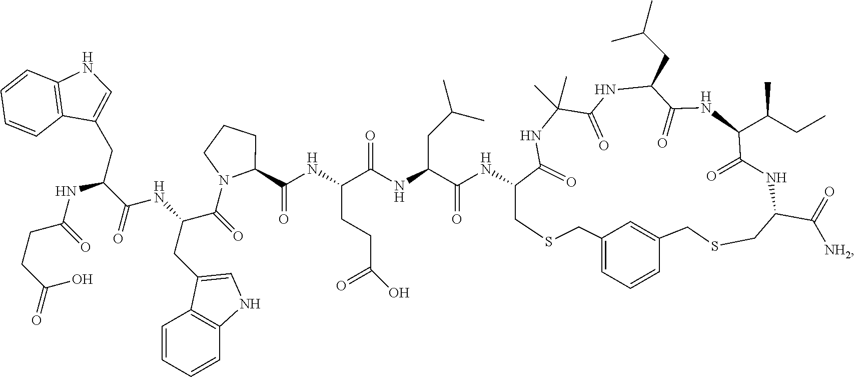 [SMILES: CC[C@H](C)[C@@H]1NC(=O)[C@H](CC(C)C)NC(=O)C(C)(C)NC(=O)[C@@H](NC(=O)[C@H](CC(C)C)NC(=O)[C@H](CCC(=O)O)NC(=O)[C@@H]2CCCN2C(=O)[C@H](Cc2c[nH]c3ccccc23)NC(=O)[C@H](Cc2c[nH]c3ccccc23)NC(=O)CCC(=O)O)CSCc2cccc(c2)CSC[C@@H](C(N)=O)NC1=O]
compound HO-Succinyl-Trp-Trp-Pro-Glu-Leu-[Cys(3MeBn)-Aib-Leu-Ile-Cys]-NH₂ (GIP-259) of the following formula 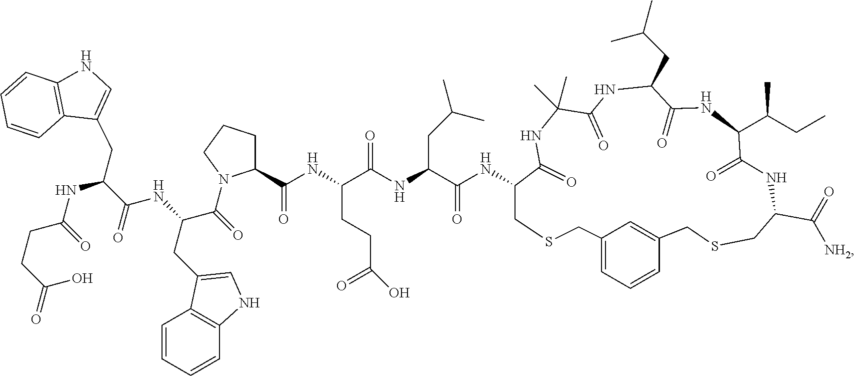 [SMILES: CC[C@H](C)[C@@H]1NC(=O)[C@H](CC(C)C)NC(=O)C(C)(C)NC(=O)[C@@H](NC(=O)[C@H](CC(C)C)NC(=O)[C@H](CCC(=O)O)NC(=O)[C@@H]2CCCN2C(=O)[C@H](Cc2c[nH]c3ccccc23)NC(=O)[C@H](Cc2c[nH]c3ccccc23)NC(=O)CCC(=O)O)CSCc2cccc(c2)CSC[C@@H](C(N)=O)NC1=O]
compound HO-Succinyl-Trp-Trp-Pro-Glu-Leu-[Cys(3MeBn)-Aib-Hle-Ile-Cys]-NH₂ (GIP-260) of the following formula 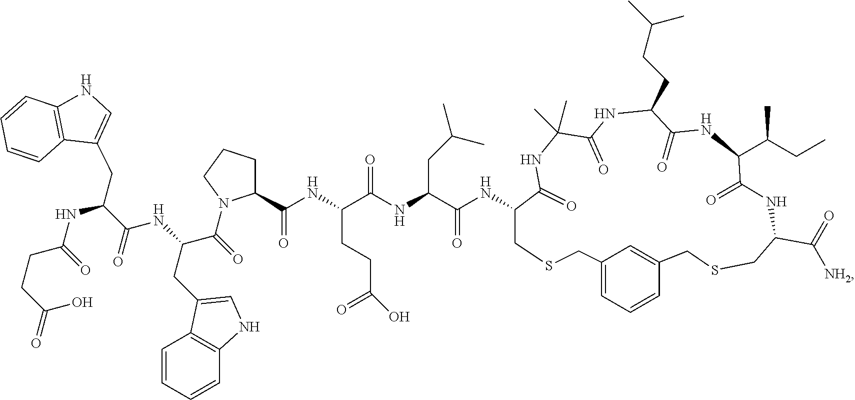 [SMILES: CC[C@H](C)[C@@H]1NC(=O)[C@H](CCC(C)C)NC(=O)C(C)(C)NC(=O)[C@@H](NC(=O)[C@H](CC(C)C)NC(=O)[C@H](CCC(=O)O)NC(=O)[C@@H]2CCCN2C(=O)[C@H](Cc2c[nH]c3ccccc23)NC(=O)[C@H](Cc2c[nH]c3ccccc23)NC(=O)CCC(=O)O)CSCc2cccc(c2)CSC[C@@H](C(N)=O)NC1=O]
compound HO-Succinyl-Trp-Trp-Pro-Glu-Leu-[Cys(3MeBn)-Thp-Leu-Ile-Cys]-NH₂ (GIP-261) of the following formula 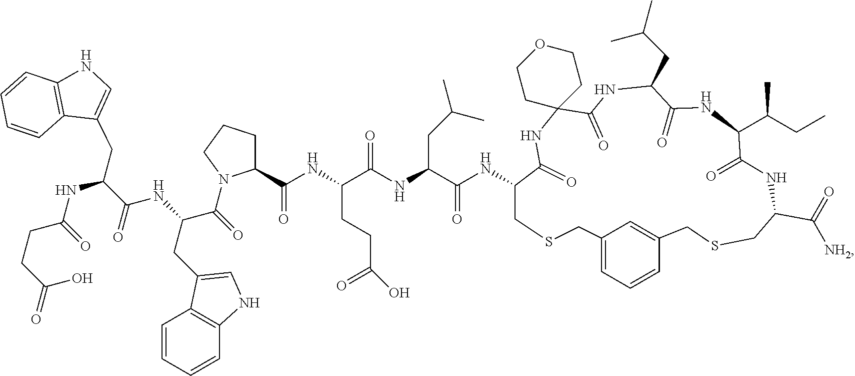 [SMILES: CC[C@H](C)[C@@H]1NC(=O)[C@H](CC(C)C)NC(=O)C2(CCOCC2)NC(=O)[C@@H](NC(=O)[C@H](CC(C)C)NC(=O)[C@H](CCC(=O)O)NC(=O)[C@@H]2CCCN2C(=O)[C@H](Cc2c[nH]c3ccccc23)NC(=O)[C@H](Cc2c[nH]c3ccccc23)NC(=O)CCC(=O)O)CSCc2cccc(c2)CSC[C@@H](C(N)=O)NC1=O]
compound Ac-Asp-Ocf-5Clw-Hyp-Glu-leu-[Cys(2Lut)-Thp-Leu-Ile-Cys]-NH₂ (GIP-262) of the 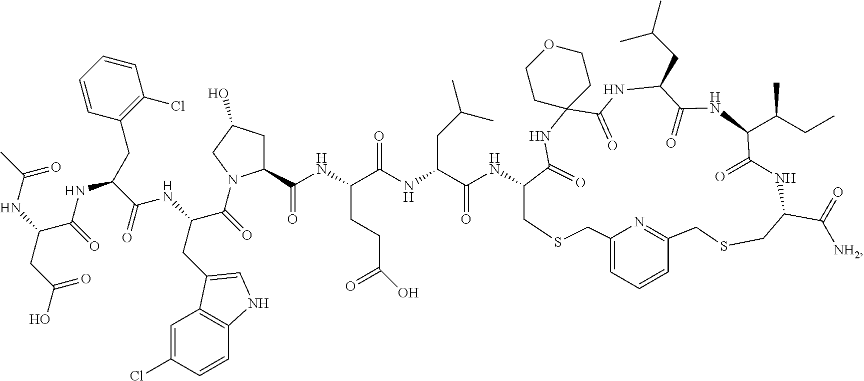 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2ccccc2Cl)NC(=O)[C@H](CC(=O)O)NC(C)=O)CSCc2cccc(n2)CSC[C@@H](C(N)=O)NC1=O]
compound Ac-Asp-Egn-5Clw-Hyp-Glu-leu-[Cys(2Lut)-Thp-Leu-Ile-Cys]-NH₂ (GIP-263) of the following formula 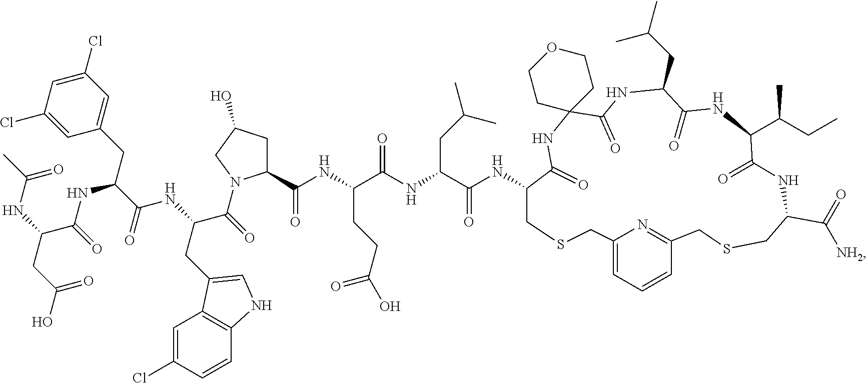 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2cc(Cl)cc(Cl)c2)NC(=O)[C@H](CC(=O)O)NC(C)=O)CSCc2cccc(n2)CSC[C@@H](C(N)=O)NC1=O]
compound Ac-Asp-Trp-Egp-Hyp-Glu-leu-[Cys(2Lut)-Thp-Leu-Ile-Cys]-NH₂ (GIP-265) of the following formula 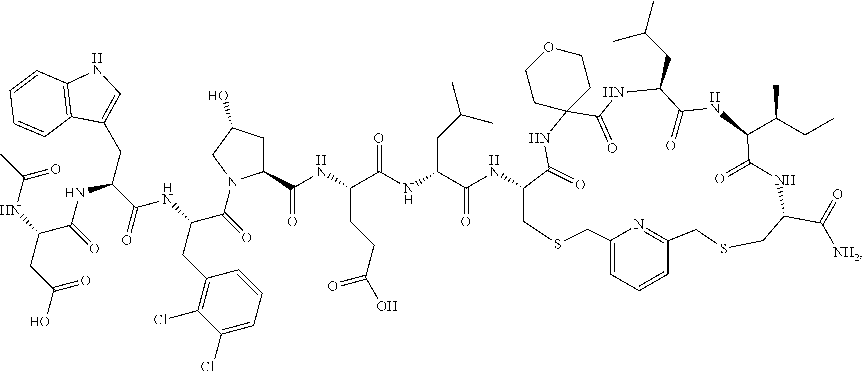 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2cccc(Cl)c2Cl)NC(=O)[C@H](Cc2c[nH]c3ccccc23)NC(=O)[C@H](CC(=O)O)NC(C)=O)CSCc2cccc(n2)CSC[C@@H](C(N)=O)NC1=O]
compound Ac-Asp-Trp-5Clw-Hyp-Glu-leu-[Cys(2Lut)-Thp-Leu-Phe-Cys]-NH₂ (GIP-266) of the following formula 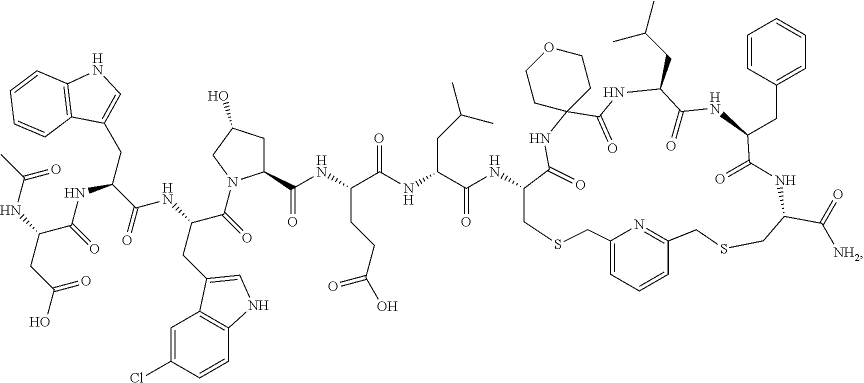 [SMILES: CC(=O)N[C@@H](CC(=O)O)C(=O)N[C@@H](Cc1c[nH]c2ccccc12)C(=O)N[C@@H](Cc1c[nH]c2ccc(Cl)cc12)C(=O)N1C[C@H](O)C[C@H]1C(=O)N[C@@H](CCC(=O)O)C(=O)N[C@H](CC(C)C)C(=O)N[C@H]1CSCc2cccc(n2)CSC[C@@H](C(N)=O)NC(=O)[C@H](Cc2ccccc2)NC(=O)[C@H](CC(C)C)NC(=O)C2(CCOCC2)NC1=O]
compound Ac-Asp-Trp-7Nw-Hyp-Glu-leu-[Cys(2Lut)-Thp-Leu-Ile-Cys]-NH₂ (GIP-267) of the 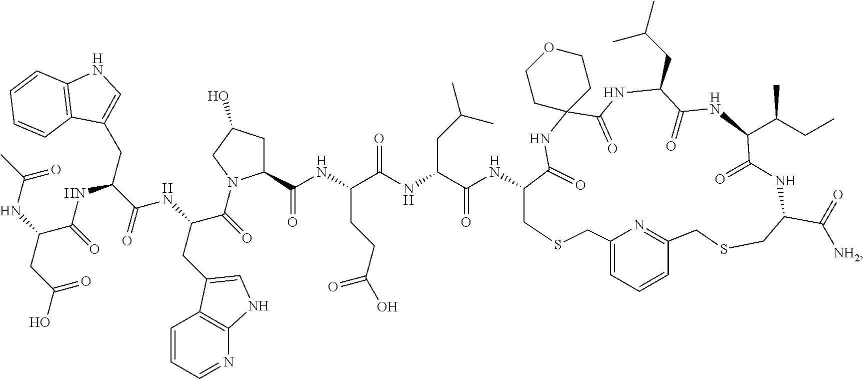 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ncccc23)NC(=O)[C@H](Cc2c[nH]c3ccccc23)NC(=O)[C@H](CC(=O)O)NC(C)=O)CSCc2cccc(n2)CSC[C@@H](C(N)=O)NC1=O]
compound DOTA-Ttds-Asp-Trp-Trp-Pro-glu-Leu-[Cys(3MeBn)-Aib-Leu-Ile-Cys]-NH₂ (GIP-270) of the following formula 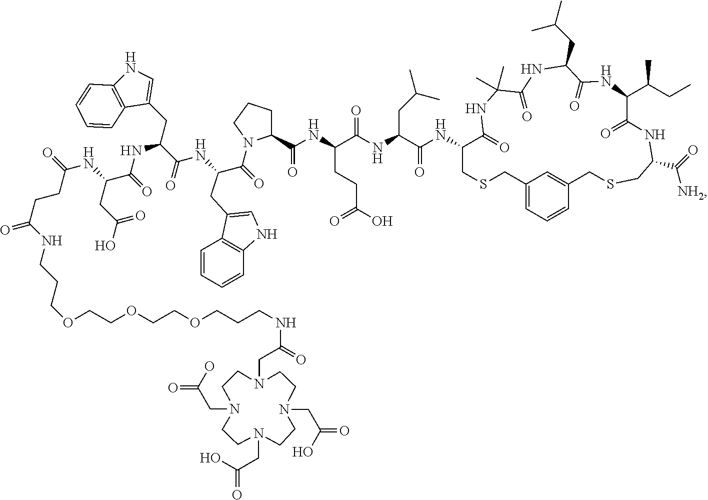
compound DOTA-Ttds-asp-Trp-Trp-Pro-Glu-Leu-[Cys(3MeBn)-ala-Leu-Ile-Cys]-NH₂ (GIP-271) of the following formula 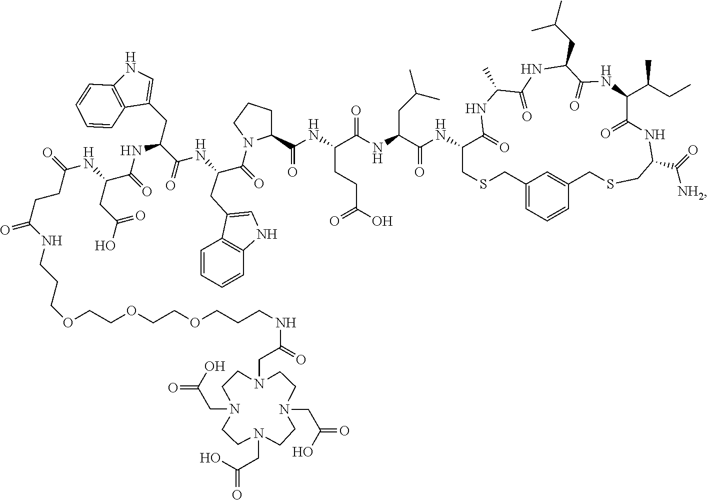 [SMILES: CC[C@H](C)[C@@H]1NC(=O)[C@H](CC(C)C)NC(=O)[C@@H](C)NC(=O)[C@@H](NC(=O)[C@H](CC(C)C)NC(=O)[C@H](CCC(=O)O)NC(=O)[C@@H]2CCCN2C(=O)[C@H](Cc2c[nH]c3ccccc23)NC(=O)[C@H](Cc2c[nH]c3ccccc23)NC(=O)[C@H](CC(=O)O)NC(=O)CCC(=O)NCCCOCCOCCOCCCNC(=O)CN2CCN(CC(=O)O)CCN(CC(=O)O)CCN(CC(=O)O)CC2)CSCc2cccc(c2)CSC[C@@H](C(N)=O)NC1=O]
compound Ac-Asp-Bta-Trp-Pro-Glu-Leu-[Cys(3MeBn)-Aib-Leu-Ile-Cys]-Ttds-Lys(DOTA)-NH₂ (GIP-273) of the following formula 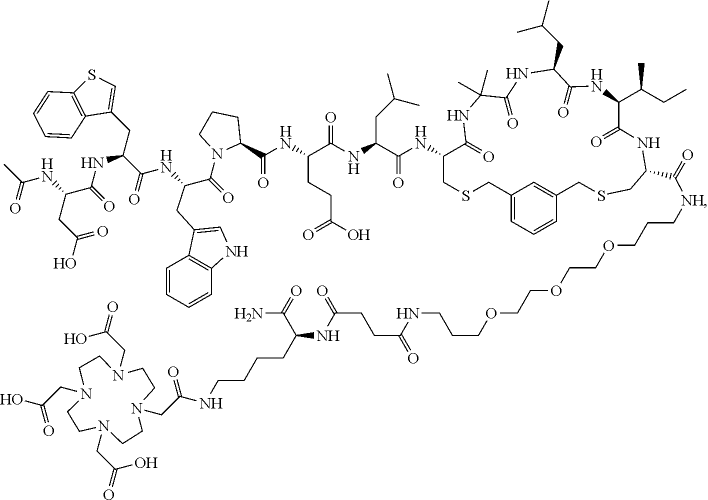 [SMILES: CC[C@H](C)[C@@H]1NC(=O)[C@H](CC(C)C)NC(=O)C(C)(C)NC(=O)[C@@H](NC(=O)[C@H](CC(C)C)NC(=O)[C@H](CCC(=O)O)NC(=O)[C@@H]2CCCN2C(=O)[C@H](Cc2c[nH]c3ccccc23)NC(=O)[C@H](Cc2csc3ccccc23)NC(=O)[C@H](CC(=O)O)NC(C)=O)CSCc2cccc(c2)CSC[C@@H](C(=O)NCCCOCCOCCOCCCNC(=O)CCC(=O)N[C@@H](CCCCNC(=O)CN2CCN(CC(=O)O)CCN(CC(=O)O)CCN(CC(=O)O)CC2)C(N)=O)NC1=O]
compound Ac-Asp-Trp-Trp-Pro-Nme-Leu-[Cys(3MeBn)-Aib-Leu-Ile-Cys]-Ttds-Lys(DOTA)-NH₂ (GIP-274) of the following formula 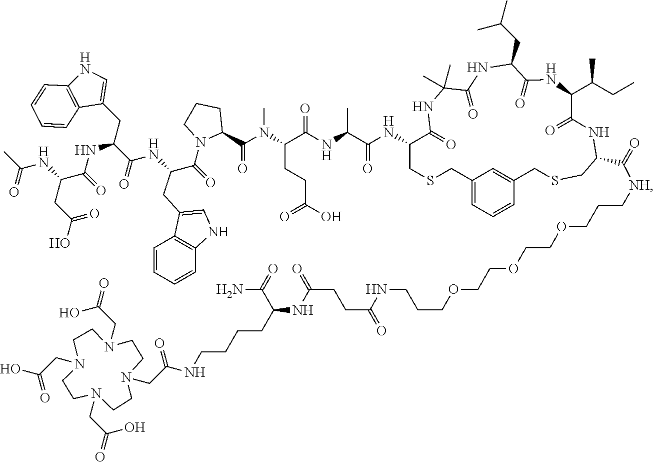 [SMILES: CC[C@H](C)[C@@H]1NC(=O)[C@H](CC(C)C)NC(=O)C(C)(C)NC(=O)[C@@H](NC(=O)[C@H](C)NC(=O)[C@H](CCC(=O)O)N(C)C(=O)[C@@H]2CCCN2C(=O)[C@H](Cc2c[nH]c3ccccc23)NC(=O)[C@H](Cc2c[nH]c3ccccc23)NC(=O)[C@H](CC(=O)O)NC(C)=O)CSCc2cccc(c2)CSC[C@@H](C(=O)NCCCOCCOCCOCCCNC(=O)CCC(=O)N[C@@H](CCCCNC(=O)CN2CCN(CC(=O)O)CCN(CC(=O)O)CCN(CC(=O)O)CC2)C(N)=O)NC1=O]
compound Ac-Asp-Trp-Trp-Pro-Glu-Leu-[Cys(3MeBn)-Aib-Tle-Ile-Cys]-Ttds-Lys(DOTA)-NH₂ (GIP-276) of the following formula 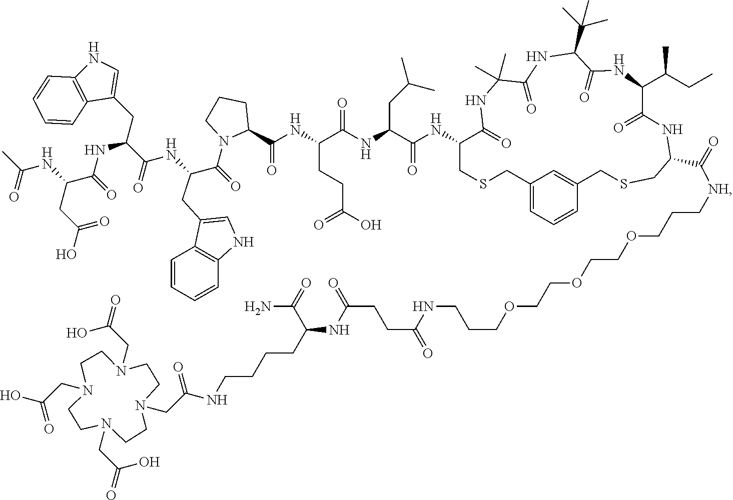 [SMILES: CC[C@H](C)[C@@H]1NC(=O)[C@H](C(C)(C)C)NC(=O)C(C)(C)NC(=O)[C@@H](NC(=O)[C@H](CC(C)C)NC(=O)[C@H](CCC(=O)O)NC(=O)[C@@H]2CCCN2C(=O)[C@H](Cc2c[nH]c3ccccc23)NC(=O)[C@H](Cc2c[nH]c3ccccc23)NC(=O)[C@H](CC(=O)O)NC(C)=O)CSCc2cccc(c2)CSC[C@@H](C(=O)NCCCOCCOCCOCCCNC(=O)CCC(=O)N[C@@H](CCCCNC(=O)CN2CCN(CC(=O)O)CCN(CC(=O)O)CCN(CC(=O)O)CC2)C(N)=O)NC1=O]
compound Ac-Asp-5Clw-Trp-Pro-Glu-Leu-[Cys(3MeBn)-Aib-Leu-Ile-Cys]-Ttds-Lys(DOTA)-NH₂ (GIP-277) of the following formula 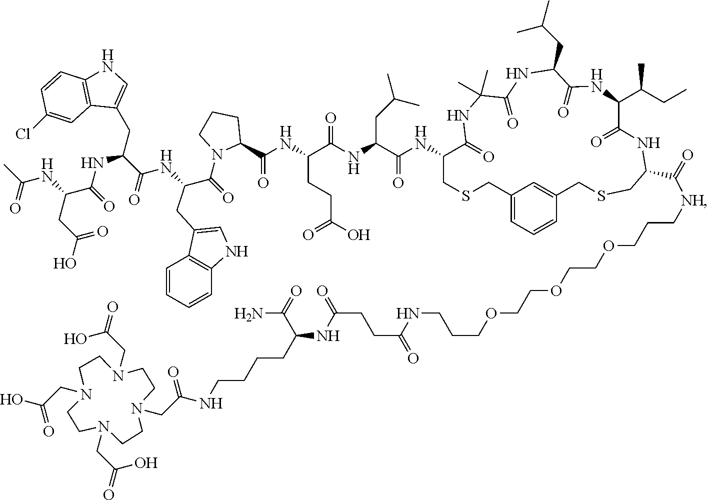 [SMILES: CC[C@H](C)[C@@H]1NC(=O)[C@H](CC(C)C)NC(=O)C(C)(C)NC(=O)[C@@H](NC(=O)[C@H](CC(C)C)NC(=O)[C@H](CCC(=O)O)NC(=O)[C@@H]2CCCN2C(=O)[C@H](Cc2c[nH]c3ccccc23)NC(=O)[C@H](Cc2c[nH]c3ccc(Cl)cc23)NC(=O)[C@H](CC(=O)O)NC(C)=O)CSCc2cccc(c2)CSC[C@@H](C(=O)NCCCOCCOCCOCCCNC(=O)CCC(=O)N[C@@H](CCCCNC(=O)CN2CCN(CC(=O)O)CCN(CC(=O)O)CCN(CC(=O)O)CC2)C(N)=O)NC1=O]
compound 4ClPhp-Asp-Trp-Trp-Pro-Glu-leu-[Cys(3MeBn)-Aib-Leu-Ile-Cys]-Ttds-Lys(DOTA)-NH₂ (GIP-279) of the following formula 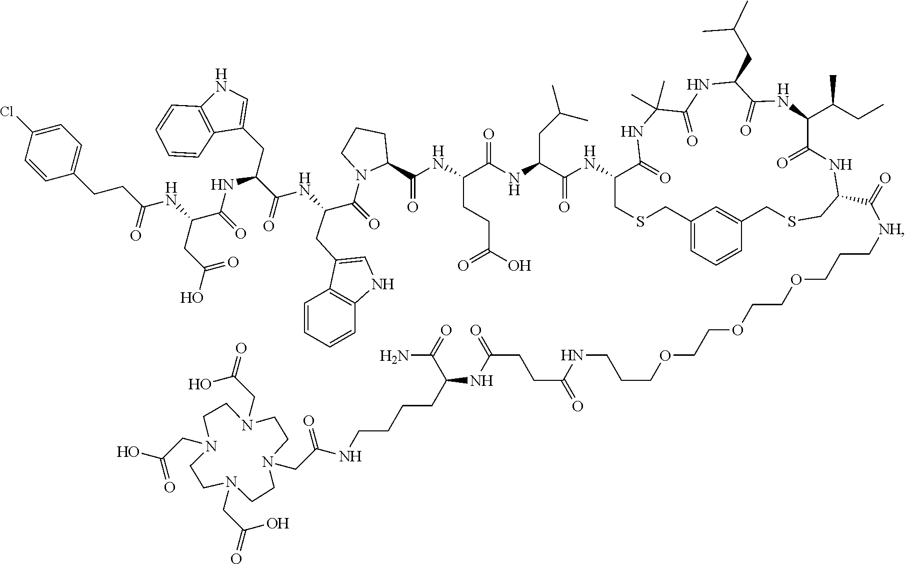 [SMILES: CC[C@H](C)[C@@H]1NC(=O)[C@H](CC(C)C)NC(=O)C(C)(C)NC(=O)[C@@H](NC(=O)[C@H](CC(C)C)NC(=O)[C@H](CCC(=O)O)NC(=O)[C@@H]2CCCN2C(=O)[C@H](Cc2c[nH]c3ccccc23)NC(=O)[C@H](Cc2c[nH]c3ccccc23)NC(=O)[C@H](CC(=O)O)NC(=O)CCc2ccc(Cl)cc2)CSCc2cccc(c2)CSC[C@@H](C(=O)NCCCOCCOCCOCCCNC(=O)CCC(=O)N[C@@H](CCCCNC(=O)CN2CCN(CC(=O)O)CCN(CC(=O)O)CCN(CC(=O)O)CC2)C(N)=O)NC1=O]
compound Nph2p-Asp-Trp-Trp-Pro-Glu-leu-[Cys(3MeBn)-Aib-Leu-Ile-Cys]-Ttds-Lys(DOTA)-NH₂ (GIP-281) of the following formula 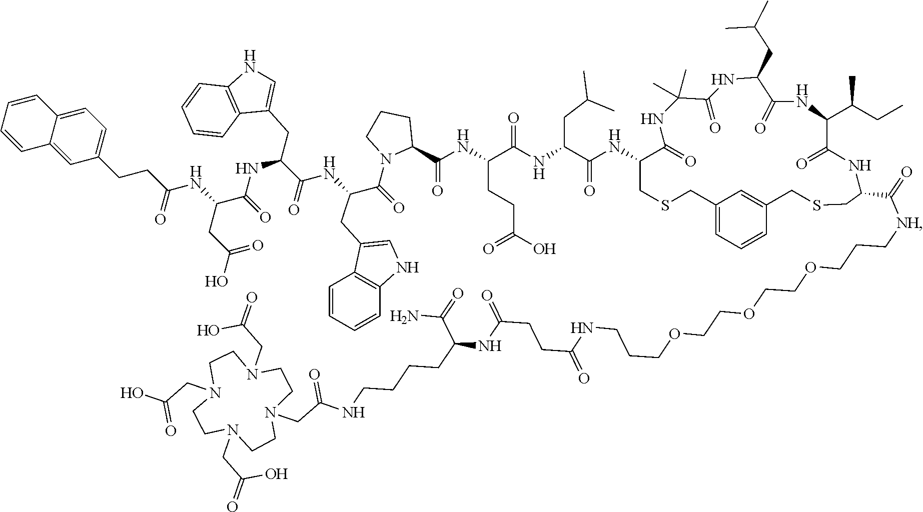 [SMILES: CC[C@H](C)[C@@H]1NC(=O)[C@H](CC(C)C)NC(=O)C(C)(C)NC(=O)[C@@H](NC(=O)[C@@H](CC(C)C)NC(=O)[C@H](CCC(=O)O)NC(=O)[C@@H]2CCCN2C(=O)[C@H](Cc2c[nH]c3ccccc23)NC(=O)[C@H](Cc2c[nH]c3ccccc23)NC(=O)[C@H](CC(=O)O)NC(=O)CCc2ccc3ccccc3c2)CSCc2cccc(c2)CSC[C@@H](C(=O)NCCCOCCOCCOCCCNC(=O)CCC(=O)N[C@@H](CCCCNC(=O)CN2CCN(CC(=O)O)CCN(CC(=O)O)CCN(CC(=O)O)CC2)C(N)=O)NC1=O]
compound Ac-Asp-Trp-6Clw-Pro-Glu-leu-[Cys(3MeBn)-Aib-Leu-Ile-Cys]-Ttds-Lys(DOTA)-NH₂ (GIP-282) of the following formula 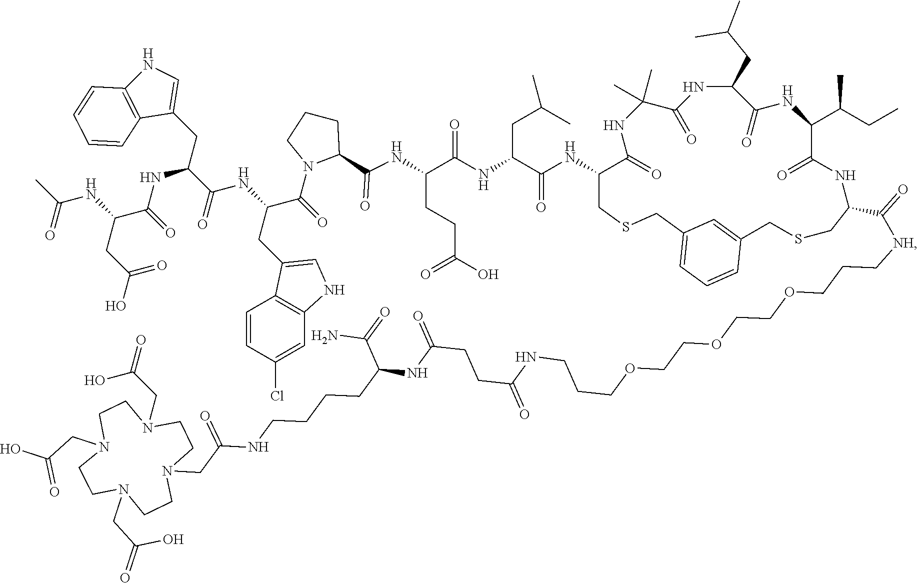 [SMILES: CC[C@H](C)[C@@H]1NC(=O)[C@H](CC(C)C)NC(=O)C(C)(C)NC(=O)[C@@H](NC(=O)[C@@H](CC(C)C)NC(=O)[C@H](CCC(=O)O)NC(=O)[C@@H]2CCCN2C(=O)[C@H](Cc2c[nH]c3cc(Cl)ccc23)NC(=O)[C@H](Cc2c[nH]c3ccccc23)NC(=O)[C@H](CC(=O)O)NC(C)=O)CSCc2cccc(c2)CSC[C@@H](C(=O)NCCCOCCOCCOCCCNC(=O)CCC(=O)N[C@@H](CCCCNC(=O)CN2CCN(CC(=O)O)CCN(CC(=O)O)CCN(CC(=O)O)CC2)C(N)=O)NC1=O]
compound DOTA-Ttds-Asp-6Fw-Trp-Pro-Glu-leu-[Cys(3MeBn)-Aib-Leu-Ile-AET] (GIP-283) of the following formula 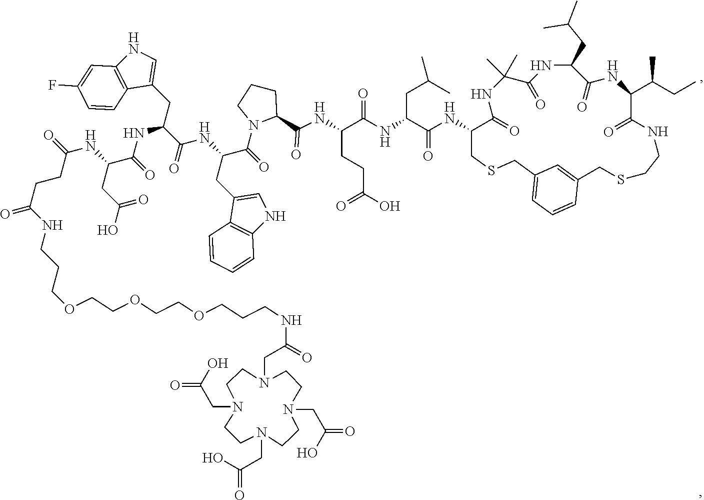 [SMILES: CC[C@H](C)[C@@H]1NC(=O)[C@H](CC(C)C)NC(=O)C(C)(C)NC(=O)[C@@H](NC(=O)[C@@H](CC(C)C)NC(=O)[C@H](CCC(=O)O)NC(=O)[C@@H]2CCCN2C(=O)[C@H](Cc2c[nH]c3ccccc23)NC(=O)[C@H](Cc2c[nH]c3cc(F)ccc23)NC(=O)[C@H](CC(=O)O)NC(=O)CCC(=O)NCCCOCCOCCOCCCNC(=O)CN2CCN(CC(=O)O)CCN(CC(=O)O)CCN(CC(=O)O)CC2)CSCc2cccc(c2)CSCCNC1=O]
compound Ac-Asp-Trp-1Ni-Pro-Glu-Hle-[Cys(3MeBn)-Aib-Leu-Ile-Cys]-NH₂ (GIP-315) of the following formula 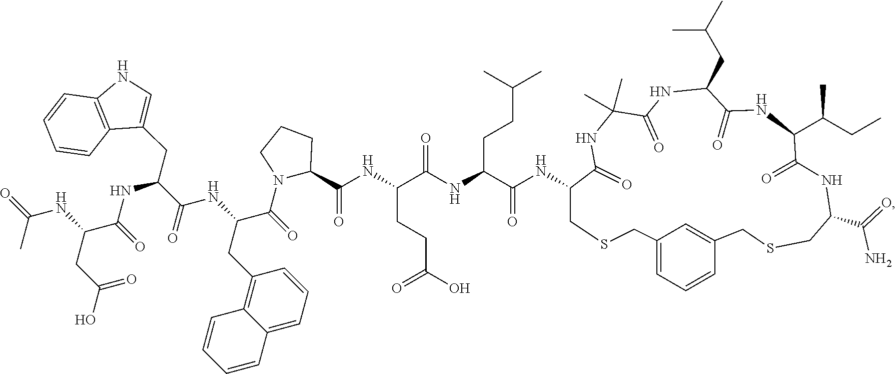 [SMILES: CC[C@H](C)[C@@H]1NC(=O)[C@H](CC(C)C)NC(=O)C(C)(C)NC(=O)[C@@H](NC(=O)[C@H](CCC(C)C)NC(=O)[C@H](CCC(=O)O)NC(=O)[C@@H]2CCCN2C(=O)[C@H](Cc2cccc3ccccc23)NC(=O)[C@H](Cc2c[nH]c3ccccc23)NC(=O)[C@H](CC(=O)O)NC(C)=O)CSCc2cccc(c2)CSC[C@@H](C(N)=O)NC1=O]
compound HO-Succinyl-Trp-Trp-Hyp-Glu-Leu-[Cys(3MeBn)-Aib-Leu-Ile-Cys]-NH₂ (GIP-319) of the following formula 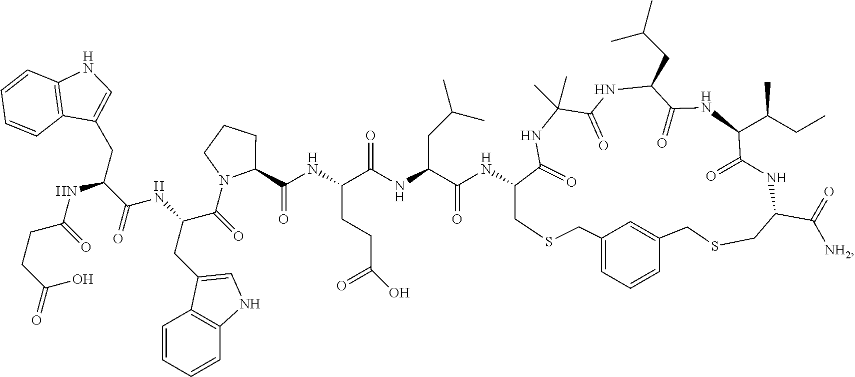 [SMILES: CC[C@H](C)[C@@H]1NC(=O)[C@H](CC(C)C)NC(=O)C(C)(C)NC(=O)[C@@H](NC(=O)[C@H](CC(C)C)NC(=O)[C@H](CCC(=O)O)NC(=O)[C@@H]2CCCN2C(=O)[C@H](Cc2c[nH]c3ccccc23)NC(=O)[C@H](Cc2c[nH]c3ccccc23)NC(=O)CCC(=O)O)CSCc2cccc(c2)CSC[C@@H](C(N)=O)NC1=O]
compound DOTA-Ttds-Asp-Trp-5Clw-Pro-Glu-leu-[Dap(3MeBz)-Aib-Leu-Ile-AET] (GIP-358) of the following formula 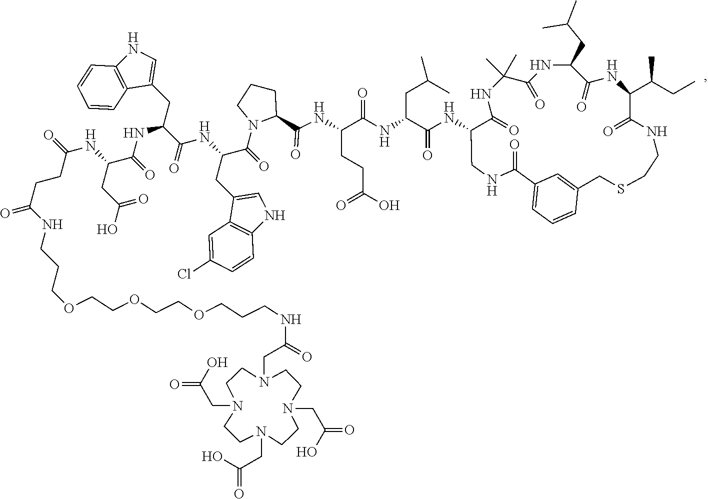 [SMILES: CC[C@H](C)[C@@H]1NC(=O)[C@H](CC(C)C)NC(=O)C(C)(C)NC(=O)[C@@H](NC(=O)[C@@H](CC(C)C)NC(=O)[C@H](CCC(=O)O)NC(=O)[C@@H]2CCCN2C(=O)[C@H](Cc2c[nH]c3ccc(Cl)cc23)NC(=O)[C@H](Cc2c[nH]c3ccccc23)NC(=O)[C@H](CC(=O)O)NC(=O)CCC(=O)NCCCOCCOCCOCCCNC(=O)CN2CCN(CC(=O)O)CCN(CC(=O)O)CCN(CC(=O)O)CC2)CNC(=O)c2cccc(c2)CSCCNC1=O]
compound DOTA-Ttds-Asp-Trp-5Clw-Pro-Glu-leu-[Cys(3CbBn)-Aib-Leu-Ile-en] (GIP-359) of the following formula 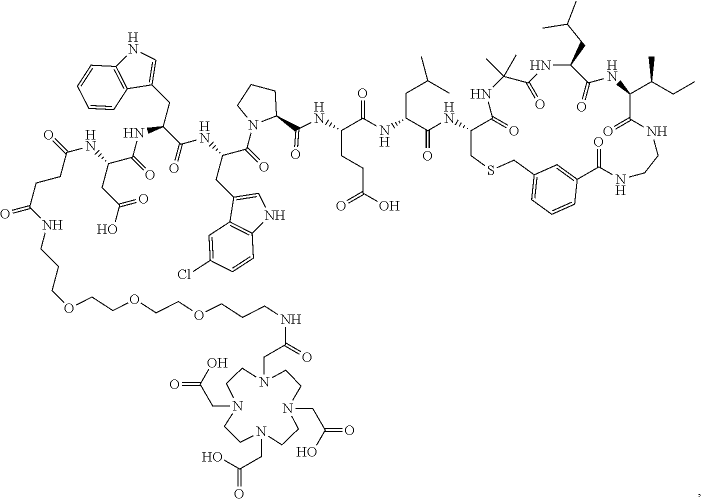 [SMILES: CC[C@H](C)[C@@H]1NC(=O)[C@H](CC(C)C)NC(=O)C(C)(C)NC(=O)[C@@H](NC(=O)[C@@H](CC(C)C)NC(=O)[C@H](CCC(=O)O)NC(=O)[C@@H]2CCCN2C(=O)[C@H](Cc2c[nH]c3ccc(Cl)cc23)NC(=O)[C@H](Cc2c[nH]c3ccccc23)NC(=O)[C@H](CC(=O)O)NC(=O)CCC(=O)NCCCOCCOCCOCCCNC(=O)CN2CCN(CC(=O)O)CCN(CC(=O)O)CCN(CC(=O)O)CC2)CSCc2cccc(c2)C(=O)NCCNC1=O]
compound Ac-Asp-Trp-5Clw-Hyp-Glu-leu-[Cys(mli)-Thp-Leu-Ile-Cys]-NH₂ (GIP-360) of the following formula 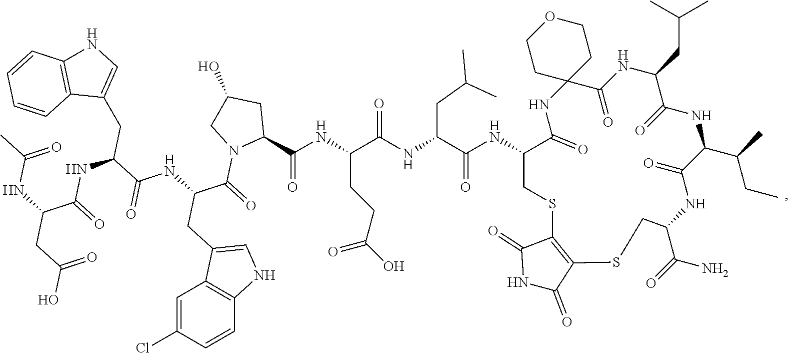 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CC(=O)O)NC(C)=O)CSC2=C(SC[C@@H](C(N)=O)NC1=O)C(=O)NC2=O]
compound Ac-Asp-Trp-5Clw-Hyp-Glu-leu-[Cys(mli)-Thp-Leu-Ile-Hcy]-NH₂ (GIP-361) of the following formula 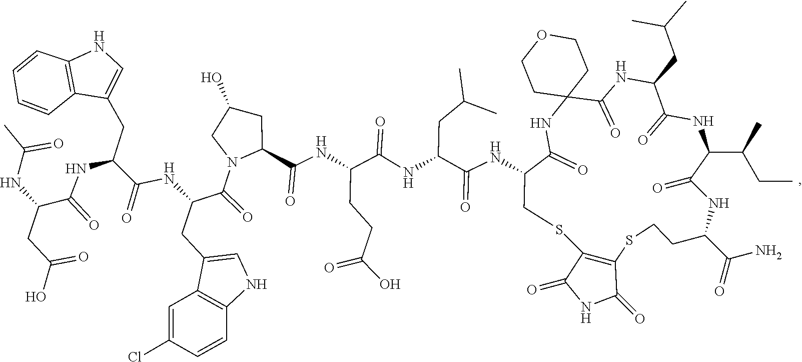 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CC(=O)O)NC(C)=O)CSC2=C(SCC[C@@H](C(N)=O)NC1=O)C(=O)NC2=O]
compound Ac-Asp-Trp-5Clw-Hyp-Glu-leu-[Hcy(mli)-Thp-Leu-Ile-Cys]-NH₂ (GIP-362) of the following formula 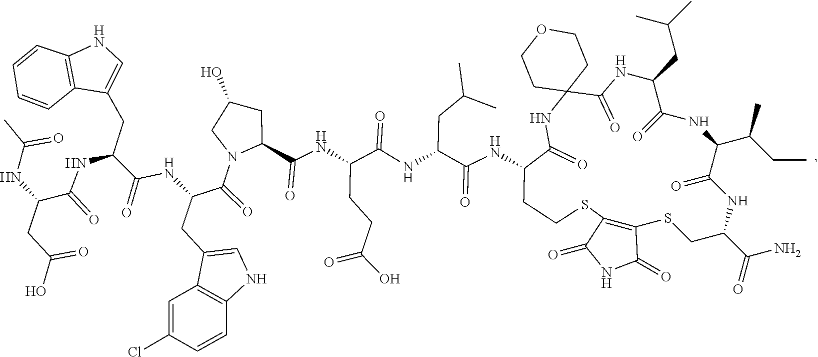 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CC(=O)O)NC(C)=O)CCSC2=C(SC[C@@H](C(N)=O)NC1=O)C(=O)NC2=O]
compound Ac-Asp-Trp-5Clw-Hyp-Glu-leu-[Hcy-Thp-Leu-Ile-Hcy]-NH₂ (GIP-363) of the following formula 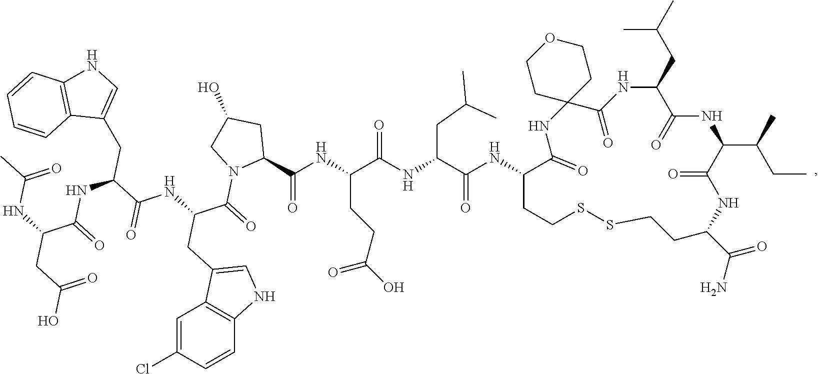 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CC(=O)O)NC(C)=O)CCSSCC[C@@H](C(N)=O)NC1=O]
compound AGLU-Glutar-Asp-Trp-5Clw-Tap-Glu-leu-[Cys(2Lut)-Apc(DOTAM)-Leu-Ile-Cys]-APAc-glu(AGLU)-NH₂ (GIP-370) of the following formula 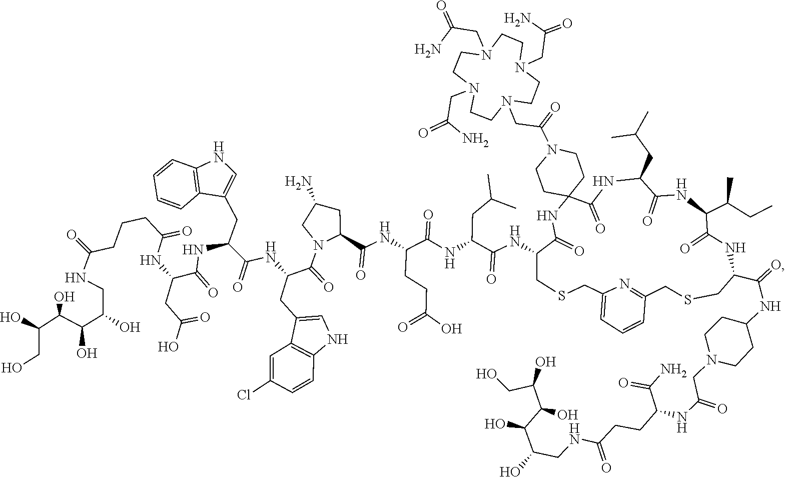 [SMILES: CC[C@H](C)[C@@H]1NC(=O)[C@H](CC(C)C)NC(=O)C2(CCN(C(=O)CN3CCN(CC(N)=O)CCN(CC(N)=O)CCN(CC(N)=O)CC3)CC2)NC(=O)[C@@H](NC(=O)[C@@H](CC(C)C)NC(=O)[C@H](CCC(=O)O)NC(=O)[C@@H]2C[C@@H](N)CN2C(=O)[C@H](Cc2c[nH]c3ccc(Cl)cc23)NC(=O)[C@H](Cc2c[nH]c3ccccc23)NC(=O)[C@H](CC(=O)O)NC(=O)CCCC(=O)NC[C@H](O)[C@@H](O)[C@H](O)[C@H](O)CO)CSCc2cccc(n2)CSC[C@@H](C(=O)NC2CCN(CC(=O)N[C@H](CCC(=O)NC[C@H](O)[C@@H](O)[C@H](O)[C@H](O)CO)C(N)=O)CC2)NC1=O]
compound AGLU-Glutar-Asp-Trp-5Clw-Tap-Glu-leu-[Cys(2Lut)-Amk(DOTA)-Leu-Ile-Cys]-APAc-glu(AGLU)-NH₂ (GIP-371) of the following formula 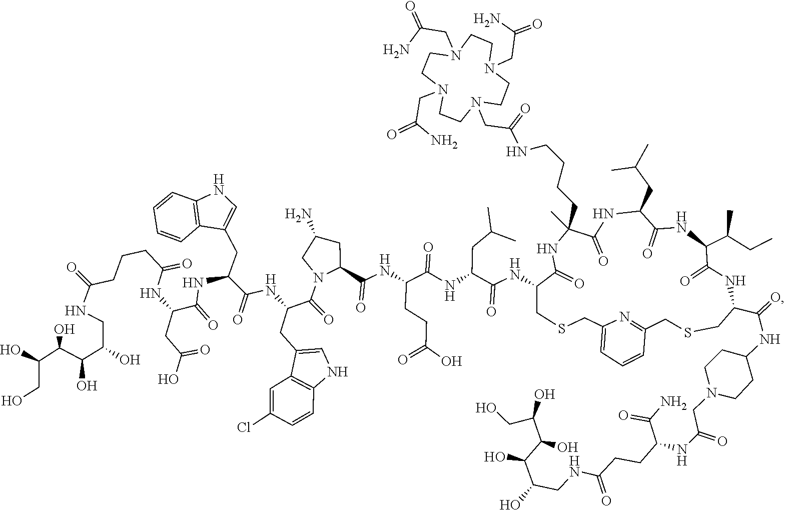 [SMILES: CC[C@H](C)[C@@H]1NC(=O)[C@H](CC(C)C)NC(=O)[C@](C)(CCCCNC(=O)CN2CCN(CC(N)=O)CCN(CC(N)=O)CCN(CC(N)=O)CC2)NC(=O)[C@@H](NC(=O)[C@@H](CC(C)C)NC(=O)[C@H](CCC(=O)O)NC(=O)[C@@H]2C[C@@H](N)CN2C(=O)[C@H](Cc2c[nH]c3ccc(Cl)cc23)NC(=O)[C@H](Cc2c[nH]c3ccccc23)NC(=O)[C@H](CC(=O)O)NC(=O)CCCC(=O)NC[C@H](O)[C@@H](O)[C@H](O)[C@H](O)CO)CSCc2cccc(n2)CSC[C@@H](C(=O)NC2CCN(CC(=O)N[C@H](CCC(=O)NC[C@H](O)[C@@H](O)[C@H](O)[C@H](O)CO)C(N)=O)CC2)NC1=O]
compound AGLU-Glutar-Asp-Trp-5Clw-Tap-Glu-leu-[Cys(2Lut)-Apc(DOTA)-Leu-Ile-Cys]-APAc-GLUSEROL (GIP-372) of the following formula 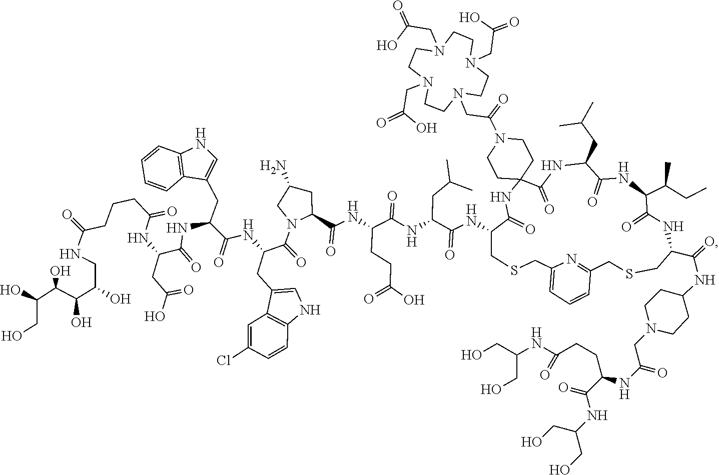 [SMILES: CC[C@H](C)[C@@H]1NC(=O)[C@H](CC(C)C)NC(=O)C2(CCN(C(=O)CN3CCN(CC(=O)O)CCN(CC(=O)O)CCN(CC(=O)O)CC3)CC2)NC(=O)[C@@H](NC(=O)[C@@H](CC(C)C)NC(=O)[C@H](CCC(=O)O)NC(=O)[C@@H]2C[C@@H](N)CN2C(=O)[C@H](Cc2c[nH]c3ccc(Cl)cc23)NC(=O)[C@H](Cc2c[nH]c3ccccc23)NC(=O)[C@H](CC(=O)O)NC(=O)CCCC(=O)NC[C@H](O)[C@@H](O)[C@H](O)[C@H](O)CO)CSCc2cccc(n2)CSC[C@@H](C(=O)NC2CCN(CC(=O)N[C@H](CCC(=O)NC(CO)CO)C(=O)NC(CO)CO)CC2)NC1=O]
compound NTASEROL-Asp-Trp-5Clw-Tap-Glu-leu-[Cys(2Lut)-Apc(DOTA)-Leu-Ile-Cys]-APAc-dap(NTASEROL)-NH₂ (GIP-373) of the following formula 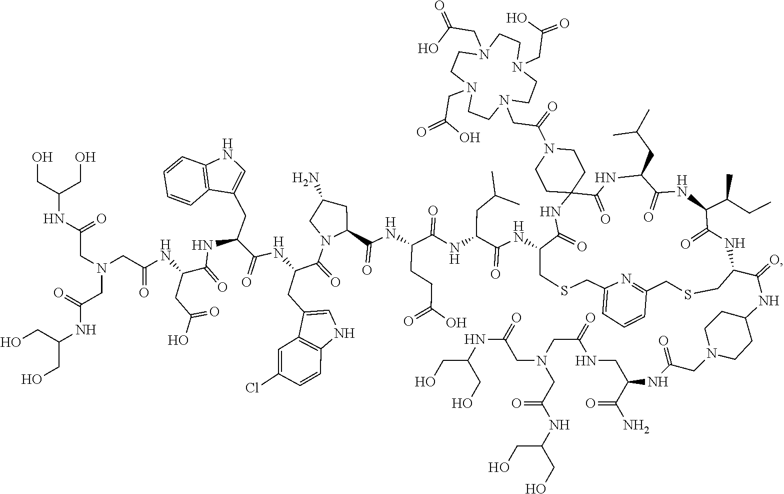 [SMILES: CC[C@H](C)[C@@H]1NC(=O)[C@H](CC(C)C)NC(=O)C2(CCN(C(=O)CN3CCN(CC(=O)O)CCN(CC(=O)O)CCN(CC(=O)O)CC3)CC2)NC(=O)[C@@H](NC(=O)[C@@H](CC(C)C)NC(=O)[C@H](CCC(=O)O)NC(=O)[C@@H]2C[C@@H](N)CN2C(=O)[C@H](Cc2c[nH]c3ccc(Cl)cc23)NC(=O)[C@H](Cc2c[nH]c3ccccc23)NC(=O)[C@H](CC(=O)O)NC(=O)CN(CC(=O)NC(CO)CO)CC(=O)NC(CO)CO)CSCc2cccc(n2)CSC[C@@H](C(=O)NC2CCN(CC(=O)N[C@H](CNC(=O)CN(CC(=O)NC(CO)CO)CC(=O)NC(CO)CO)C(N)=O)CC2)NC1=O]
compound NTASEROL-Asp-Trp-5Clw-Tap-Glu-leu-[Cys(2Lut)-Apc(DOTA)-Leu-Ile-Cys]-APAc-GLUSEROL (GIP-374) of the following formula 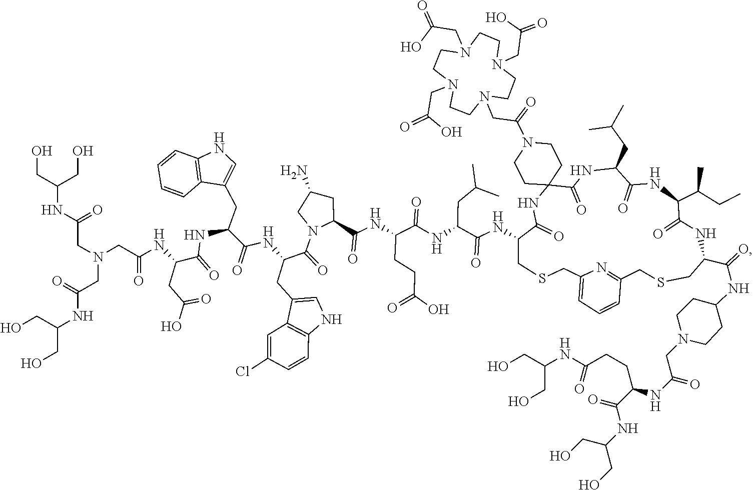 [SMILES: CC[C@H](C)[C@@H]1NC(=O)[C@H](CC(C)C)NC(=O)C2(CCN(C(=O)CN3CCN(CC(=O)O)CCN(CC(=O)O)CCN(CC(=O)O)CC3)CC2)NC(=O)[C@@H](NC(=O)[C@@H](CC(C)C)NC(=O)[C@H](CCC(=O)O)NC(=O)[C@@H]2C[C@@H](N)CN2C(=O)[C@H](Cc2c[nH]c3ccc(Cl)cc23)NC(=O)[C@H](Cc2c[nH]c3ccccc23)NC(=O)[C@H](CC(=O)O)NC(=O)CN(CC(=O)NC(CO)CO)CC(=O)NC(CO)CO)CSCc2cccc(n2)CSC[C@@H](C(=O)NC2CCN(CC(=O)N[C@H](CCC(=O)NC(CO)CO)C(=O)NC(CO)CO)CC2)NC1=O]
compound Ac-Asp-Trp-5Clw-Hyp-Lys-leu-[Cys(2Lut)-Thp-Leu-Ile-Cys]-NH₂ (GIP-375) of the following formula 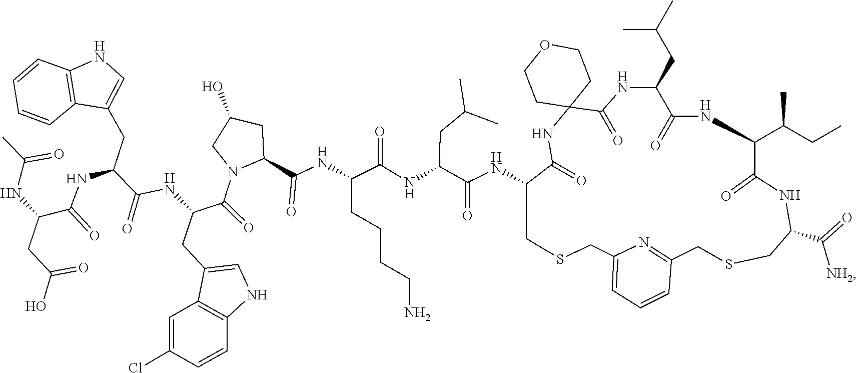 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCCCN)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CC(=O)O)NC(C)=O)CSCc2cccc(n2)CSC[C@@H](C(N)=O)NC1=O]
compound Ac-Asp-Trp-5Clw-Hyp-Arg-leu-[Cys(2Lut)-Thp-Leu-Ile-Cys]-NH₂ (GIP-376) of the following formula 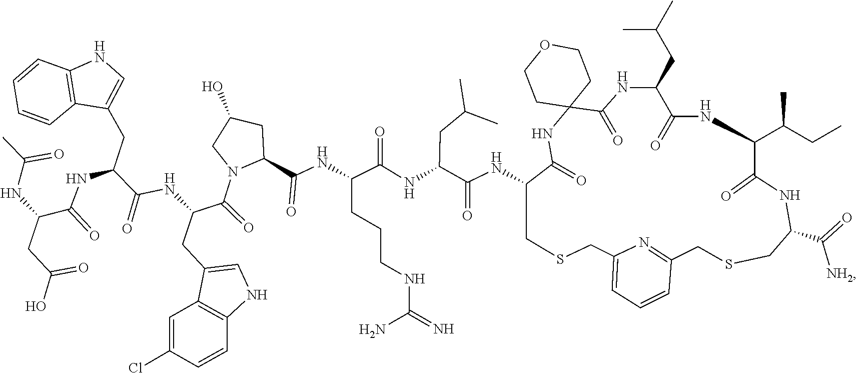 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCCNC(=N)N)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CC(=O)O)NC(C)=O)CSCc2cccc(n2)CSC[C@@H](C(N)=O)NC1=O]
compound Ac-Asp-Trp-5Clw-Hyp-His-leu-[Cys(2Lut)-Thp-Leu-Ile-Cys]-NH₂ (GIP-377) of the following formula 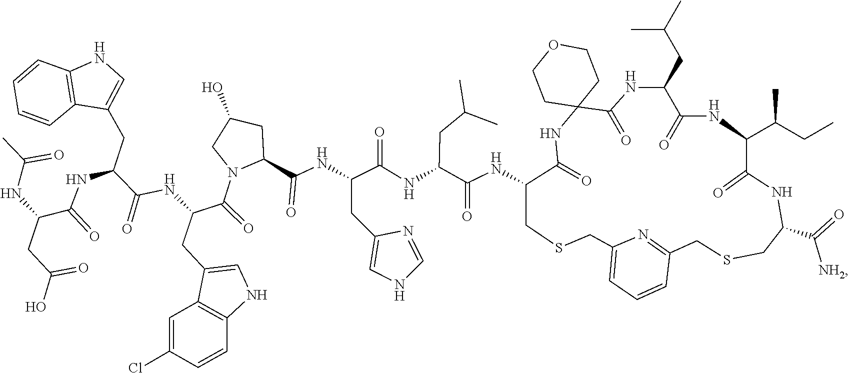 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](Cc2c[nH]cn2)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CC(=O)O)NC(C)=O)CSCc2cccc(n2)CSC[C@@H](C(N)=O)NC1=O]
compound Ac-Asp-Trp-5Clw-Hyp-Hly-leu-[Cys(2Lut)-Thp-Leu-Ile-Cys]-NH₂ (GIP-378) of the following formula 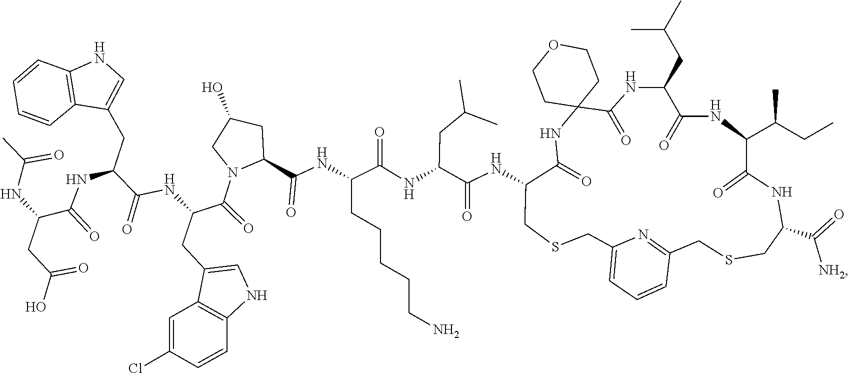 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCCCCN)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CC(=O)O)NC(C)=O)CSCc2cccc(n2)CSC[C@@H](C(N)=O)NC1=O]
compound Ac-Asp-5Clw-5Clw-Hyp-Glu-leu-[Cys(2Lut)-Thp-Leu-Ile-Cys]-NH₂ (GIP-379) of the following formula 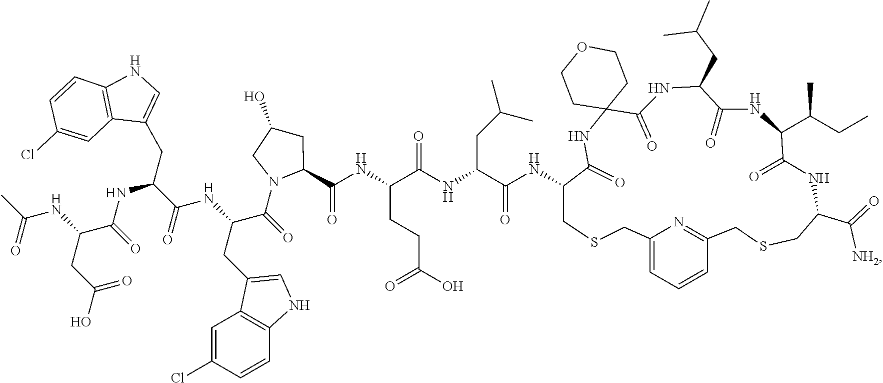 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3ccc(Cl)cc23)NC(=O)[C@H](CC(=O)O)NC(C)=O)CSCc2cccc(n2)CSC[C@@H](C(N)=O)NC1=O]
compound Ac-Asp-1Ni-5Clw-Hyp-Glu-leu-[Cys(2Lut)-Thp-Leu-Ile-Cys]-NH₂ (GIP-380) of the 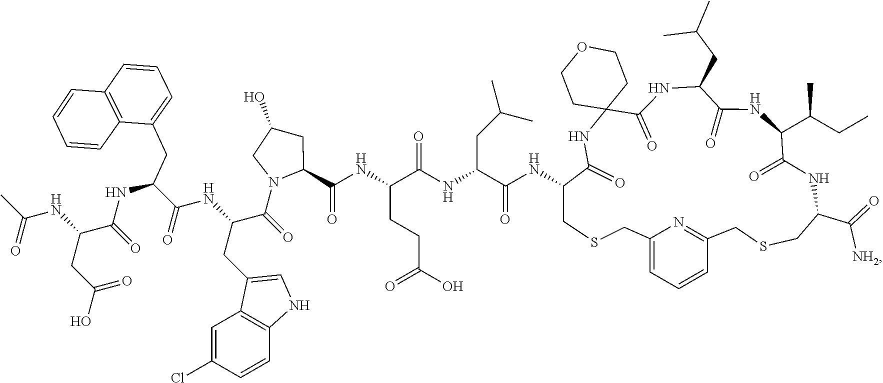 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2cccc3ccccc23)NC(=O)[C@H](CC(=O)O)NC(C)=O)CSCc2cccc(n2)CSC[C@@H](C(N)=O)NC1=O]
compound Ac-asp-Trp-5Clw-Hyp-Glu-leu-[Cys(2Lut)-Thp-Leu-Ile-Cys]-NH₂ (GIP-381) of the following formula 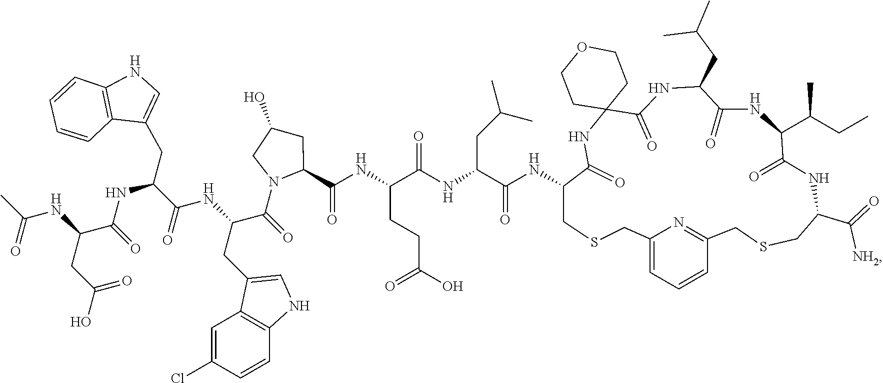 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@@H](CC(=O)O)NC(C)=O)CSCc2cccc(n2)CSC[C@@H](C(N)=O)NC1=O]
compound Ac-Asp-5Fw-5Clw-Hyp-Glu-leu-[Cys(2Lut)-Thp-Leu-Ile-Cys]-NH₂ (GIP-382) of the following formula 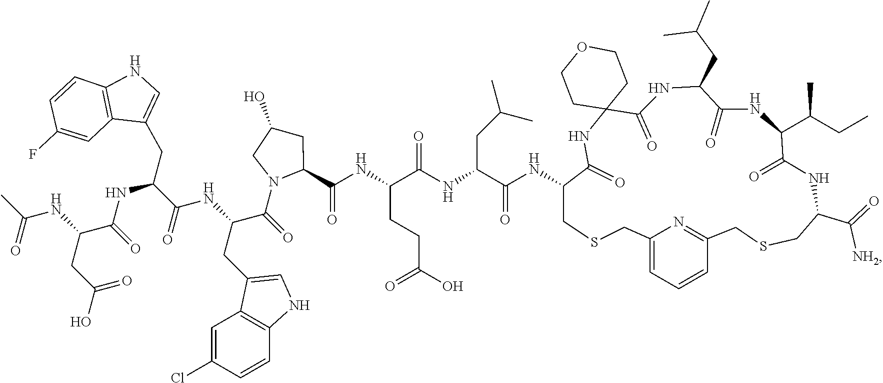 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3ccc(F)cc23)NC(=O)[C@H](CC(=O)O)NC(C)=O)CSCc2cccc(n2)CSC[C@@H](C(N)=O)NC1=O]
compound Ac-Asp-Trp-5Clw-Hyp-Glu-leu-[Cys(2Lut)-Thp-Cha-Ile-Cys]-NH₂ (GIP-383) of the following formula 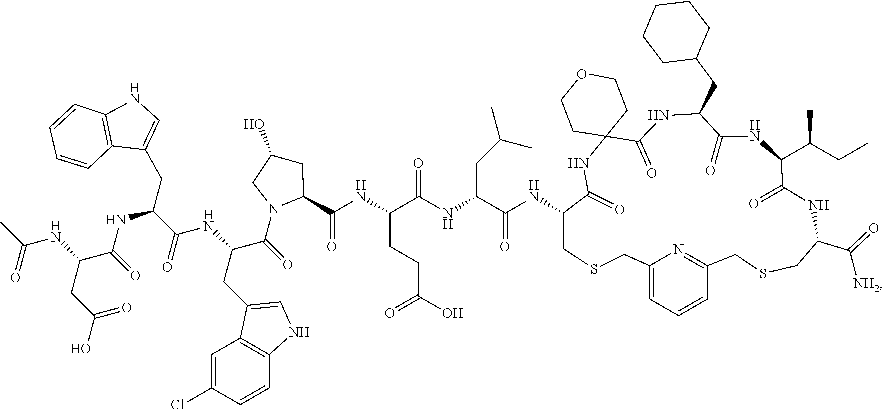 [SMILES: CC[C@H](C)[C@@H]1NC(=O)[C@H](CC2CCCCC2)NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CC(=O)O)NC(C)=O)CSCc2cccc(n2)CSC[C@@H](C(N)=O)NC1=O]
compound Ac-Asp-Trp-5Clw-Hyp-Ser-leu-[Cys(2Lut)-Thp-Leu-Ile-Cys]-NH₂ (GIP-384) of the following formula 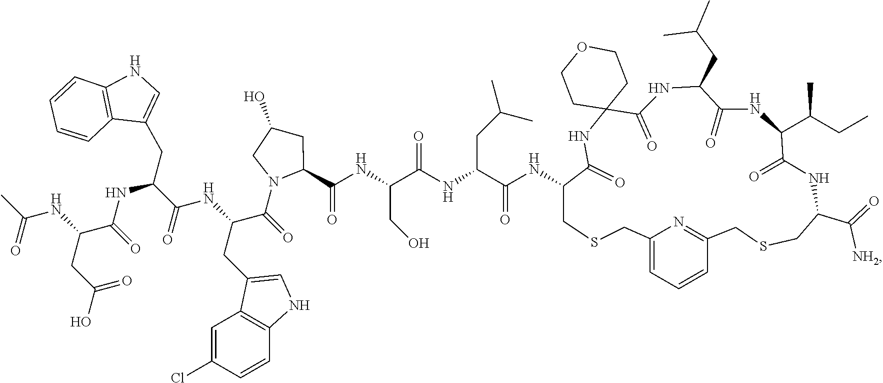 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CO)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CC(=O)O)NC(C)=O)CSCc2cccc(n2)CSC[C@@H](C(N)=O)NC1=O]
compound Ac-Asp-Trp-5Clw-Hyp-Val-leu-[Cys(2Lut)-Thp-Leu-Ile-Cys]-NH₂ (GIP-385) of the following formula 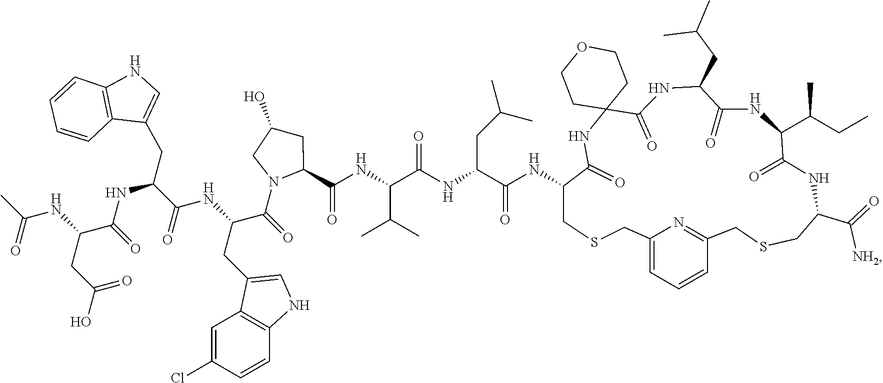 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@@H](NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CC(=O)O)NC(C)=O)C(C)C)CSCc2cccc(n2)CSC[C@@H](C(N)=O)NC1=O]
compound Ac-Asp-Trp-5Clw-Hyp-Ala-leu-[Cys(2Lut)-Thp-Leu-Ile-Cys]-NH₂ (GIP-386) of the following formula 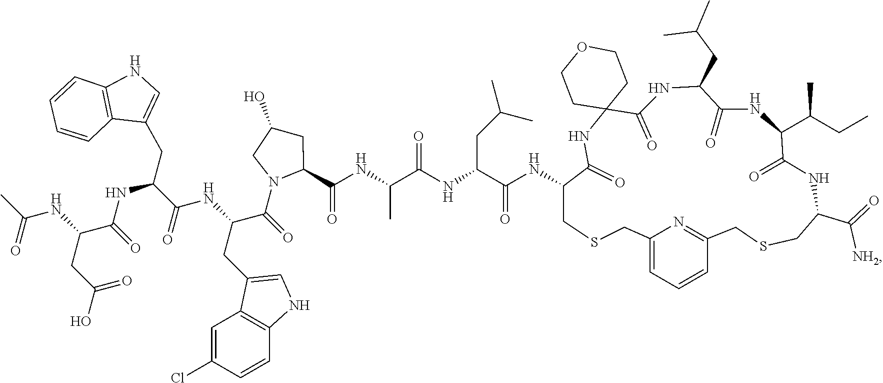 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](C)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CC(=O)O)NC(C)=O)CSCc2cccc(n2)CSC[C@@H](C(N)=O)NC1=O]
compound DOTA-Ttds-Asp-Ocf-5Clw-Hyp-Glu-leu-[Cys(2Lut)-Thp-Leu-Ile-Cys]-NH₂ (GIP-387) of the following formula 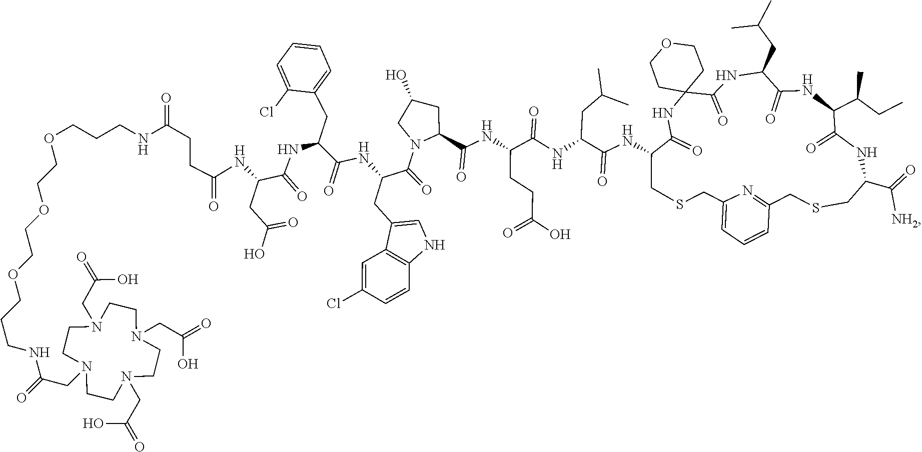 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2ccccc2Cl)NC(=O)[C@H](CC(=O)O)NC(=O)CCC(=O)NCCCOCCOCCOCCCNC(=O)CN2CCN(CC(=O)O)CCN(CC(=O)O)CCN(CC(=O)O)CC2)CSCc2cccc(n2)CSC[C@@H](C(N)=O)NC1=O]
compound DOTA-Ttds-Asp-Trp-5Clw-Hyp-Glu-leu-[Cys(2Lut)-Thp-Leu-Phe-Cys]-NH₂ (GIP-388) of the following formula 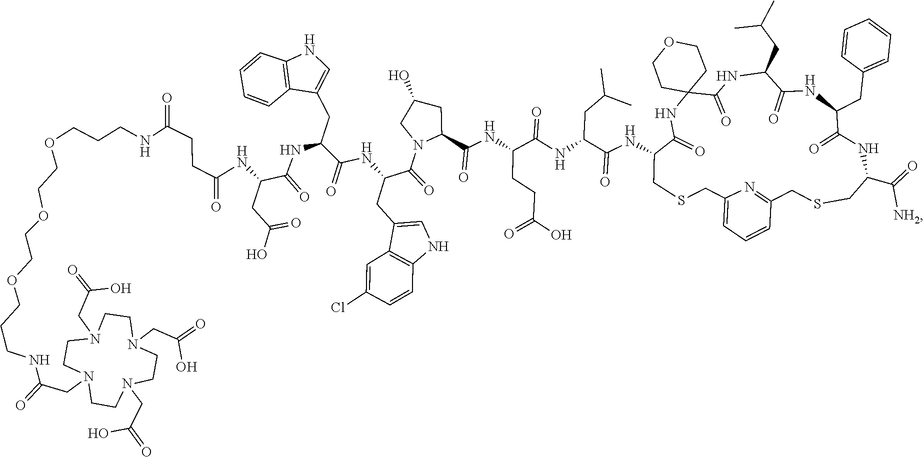 [SMILES: CC(C)C[C@@H]1NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CC(=O)O)NC(=O)CCC(=O)NCCCOCCOCCOCCCNC(=O)CN2CCN(CC(=O)O)CCN(CC(=O)O)CCN(CC(=O)O)CC2)CSCc2cccc(n2)CSC[C@@H](C(N)=O)NC(=O)[C@H](Cc2ccccc2)NC1=O]
compound Ac-Asp-Trp-5Clw-Hyp-Glu-leu-[Cys(2Lut)-Thp-Leu-Phe-Cys]-lys(DOTA)-NH₂ (GIP-389) of the following formula 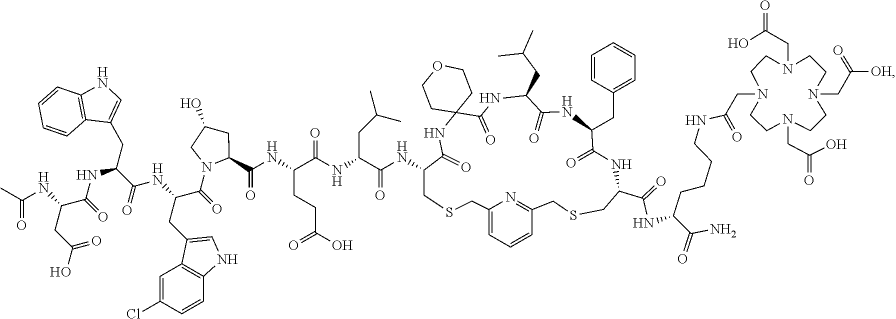 [SMILES: CC(=O)N[C@@H](CC(=O)O)C(=O)N[C@@H](Cc1c[nH]c2ccccc12)C(=O)N[C@@H](Cc1c[nH]c2ccc(Cl)cc12)C(=O)N1C[C@H](O)C[C@H]1C(=O)N[C@@H](CCC(=O)O)C(=O)N[C@H](CC(C)C)C(=O)N[C@H]1CSCc2cccc(n2)CSC[C@@H](C(=O)N[C@H](CCCCNC(=O)CN2CCN(CC(=O)O)CCN(CC(=O)O)CCN(CC(=O)O)CC2)C(N)=O)NC(=O)[C@H](Cc2ccccc2)NC(=O)[C@H](CC(C)C)NC(=O)C2(CCOCC2)NC1=O]
compound Ac-Asp-Ocf-5Clw-Tap(DOTA)-Glu-leu-[Cys(2Lut)-Thp-Leu-Ile-Cys]-NH₂ (GIP-390) of the following formula 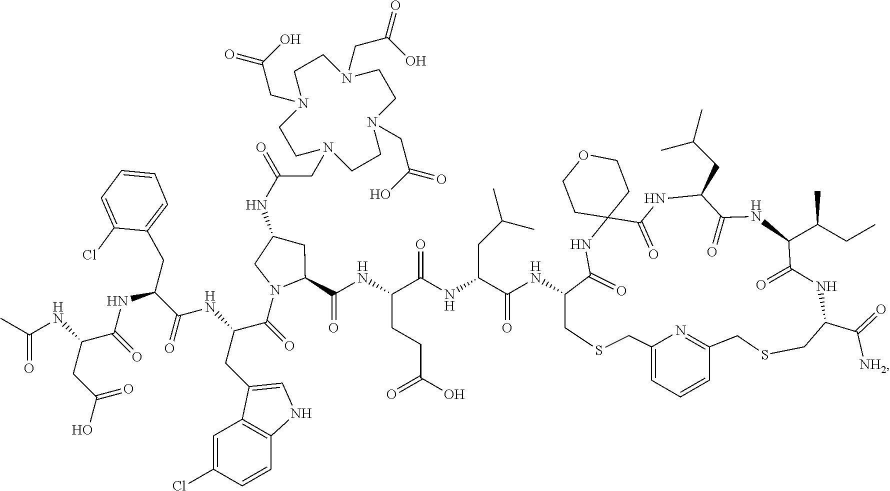 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](NC(=O)CN3CCN(CC(=O)O)CCN(CC(=O)O)CCN(CC(=O)O)CC3)CN2C(=O)[C@H](Cc2c[nH]c3ccc(Cl)cc23)NC(=O)[C@H](Cc2ccccc2Cl)NC(=O)[C@H](CC(=O)O)NC(C)=O)CSCc2cccc(n2)CSC[C@@H](C(N)=O)NC1=O]
compound Ac-Asp-Trp-5Clw-Tap(DOTA)-Glu-leu-[Cys(2Lut)-Thp-Leu-Phe-Cys]-NH₂ (GIP-391) of the following formula 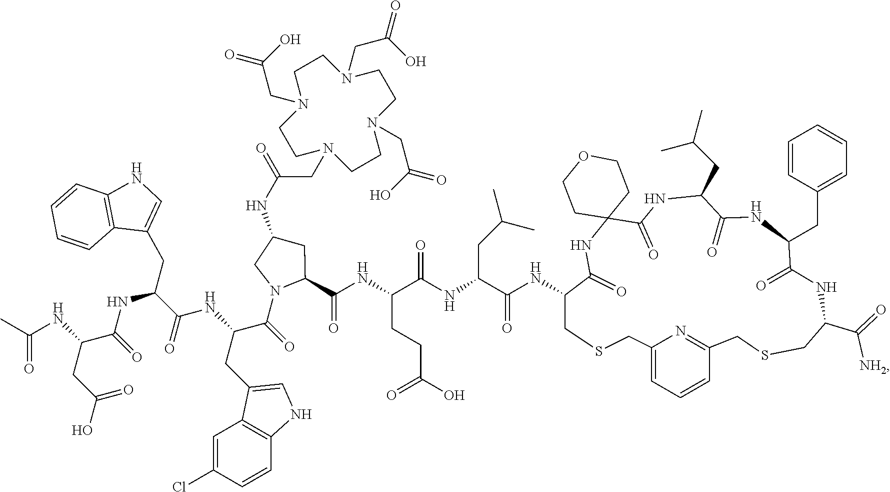 [SMILES: CC(=O)N[C@@H](CC(=O)O)C(=O)N[C@@H](Cc1c[nH]c2ccccc12)C(=O)N[C@@H](Cc1c[nH]c2ccc(Cl)cc12)C(=O)N1C[C@H](NC(=O)CN2CCN(CC(=O)O)CCN(CC(=O)O)CCN(CC(=O)O)CC2)C[C@H]1C(=O)N[C@@H](CCC(=O)O)C(=O)N[C@H](CC(C)C)C(=O)N[C@H]1CSCc2cccc(n2)CSC[C@@H](C(N)=O)NC(=O)[C@H](Cc2ccccc2)NC(=O)[C@H](CC(C)C)NC(=O)C2(CCOCC2)NC1=O]
compound Ac-Asp-Ocf-5Clw-Hyp-Glu-leu-[Cys(2Lut)-Apc(DOTA)-Leu-Ile-Cys]-NH₂ (GIP-392) of the following formula 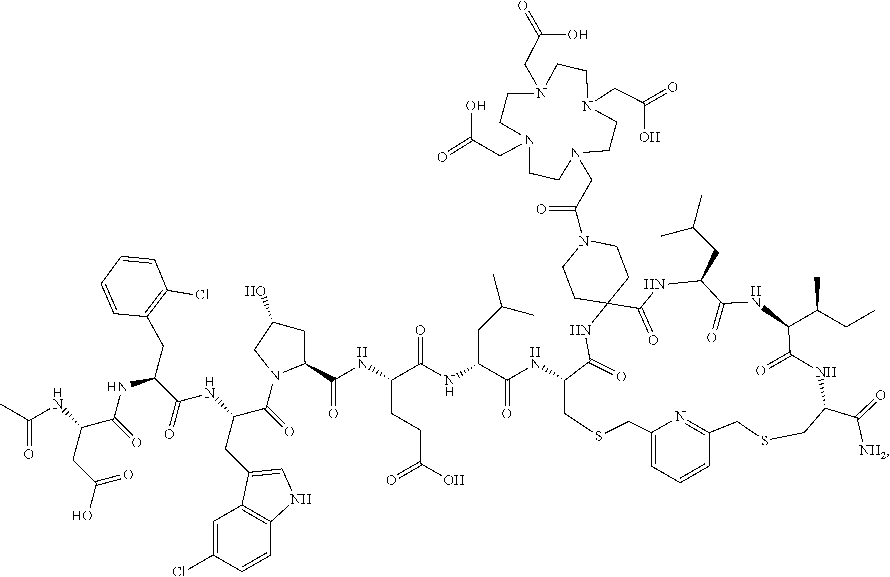 [SMILES: CC[C@H](C)[C@@H]1NC(=O)[C@H](CC(C)C)NC(=O)C2(CCN(C(=O)CN3CCN(CC(=O)O)CCN(CC(=O)O)CCN(CC(=O)O)CC3)CC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2ccccc2Cl)NC(=O)[C@H](CC(=O)O)NC(C)=O)CSCc2cccc(n2)CSC[C@@H](C(N)=O)NC1=O]
compound Ac-Asp-Trp-5Clw-Hyp-Glu-leu-[Cys(2Lut)-Apc(DOTA)-Leu-Phe-Cys]-NH₂ (GIP-393) of the following formula 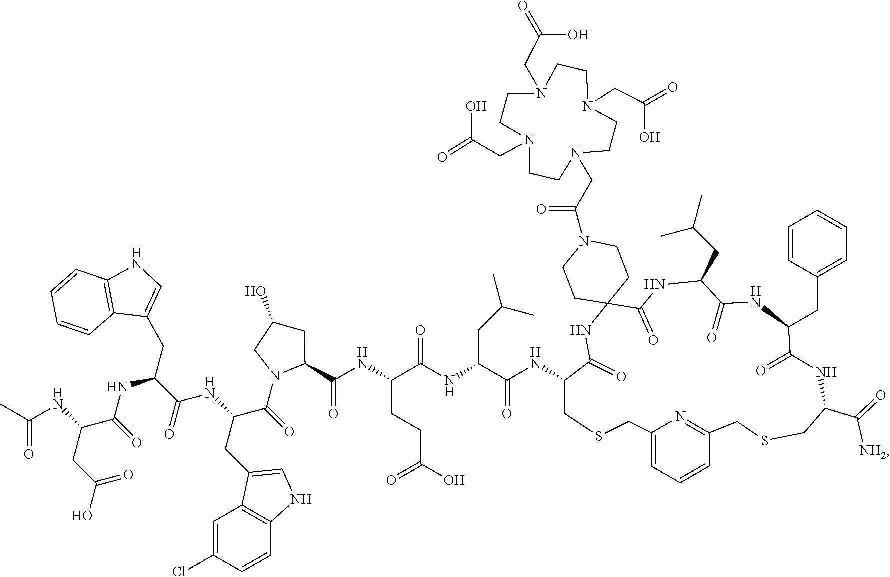 [SMILES: CC(=O)N[C@@H](CC(=O)O)C(=O)N[C@@H](Cc1c[nH]c2ccccc12)C(=O)N[C@@H](Cc1c[nH]c2ccc(Cl)cc12)C(=O)N1C[C@H](O)C[C@H]1C(=O)N[C@@H](CCC(=O)O)C(=O)N[C@H](CC(C)C)C(=O)N[C@H]1CSCc2cccc(n2)CSC[C@@H](C(N)=O)NC(=O)[C@H](Cc2ccccc2)NC(=O)[C@H](CC(C)C)NC(=O)C2(CCN(C(=O)CN3CCN(CC(=O)O)CCN(CC(=O)O)CCN(CC(=O)O)CC3)CC2)NC1=O]
compound Ac-Asp-Trp-1Ni-Tap(DOTA)-Glu-Hle-[Cys(3MeBn)-Aib-Leu-Ile-Cys]-NH₂ (GIP-394) of the following formula 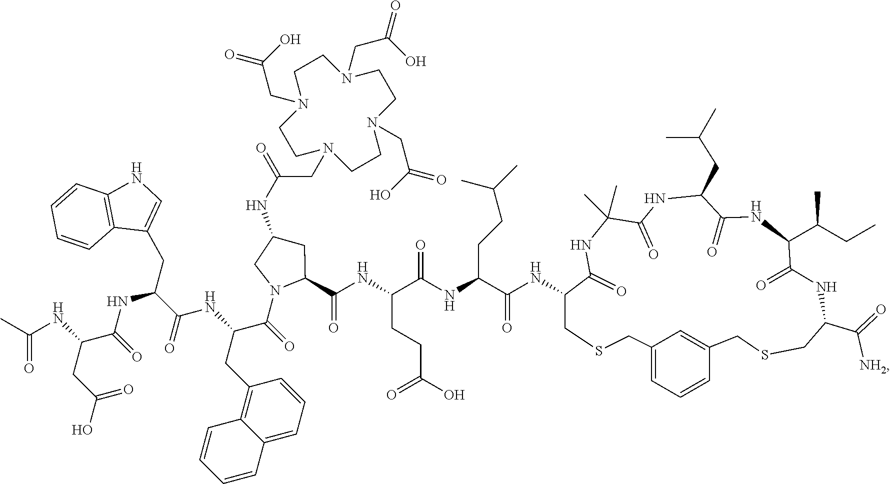 [SMILES: CC[C@H](C)[C@@H]1NC(=O)[C@H](CC(C)C)NC(=O)C(C)(C)NC(=O)[C@@H](NC(=O)[C@H](CCC(C)C)NC(=O)[C@H](CCC(=O)O)NC(=O)[C@@H]2C[C@@H](NC(=O)CN3CCN(CC(=O)O)CCN(CC(=O)O)CCN(CC(=O)O)CC3)CN2C(=O)[C@H](Cc2cccc3ccccc23)NC(=O)[C@H](Cc2c[nH]c3ccccc23)NC(=O)[C@H](CC(=O)O)NC(C)=O)CSCc2cccc(c2)CSC[C@@H](C(N)=O)NC1=O]
compound Ac-Asp-Trp-5Clw-Hyp-Har-leu-[Cys(2Lut)-Thp-Leu-Ile-Cys]-NH₂ (GIP-395) of the following formula 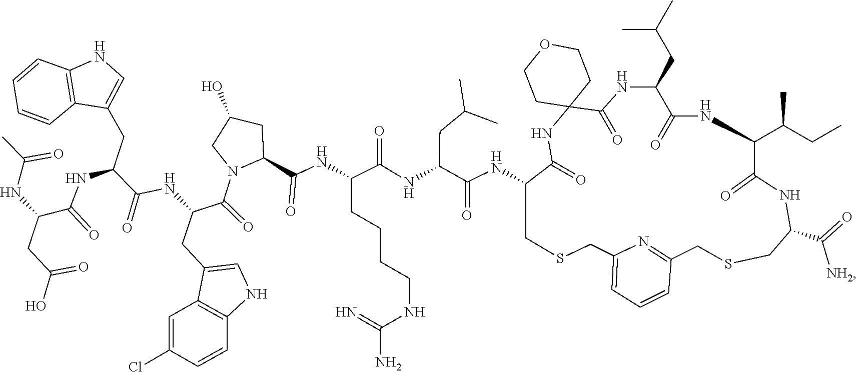 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCCCNC(=N)N)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CC(=O)O)NC(C)=O)CSCc2cccc(n2)CSC[C@@H](C(N)=O)NC1=O]
compound Ac-Asp-Trp-5Clw-Hyp-Phe-leu-[Cys(2Lut)-Thp-Leu-Ile-Cys]-NH₂ (GIP-396) of the following formula 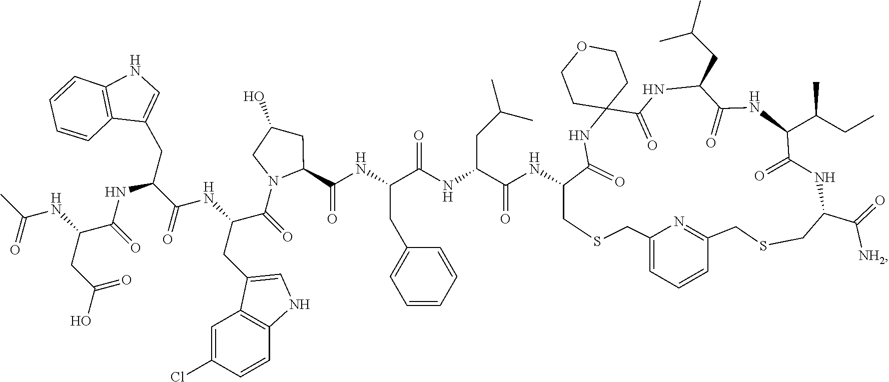 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](Cc2ccccc2)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CC(=O)O)NC(C)=O)CSCc2cccc(n2)CSC[C@@H](C(N)=O)NC1=O]
compound Ac-Asp-Trp-5Clw-Hyp-Trp-leu-[Cys(2Lut)-Thp-Leu-Ile-Cys]-NH₂ (GIP-397) of the following formula 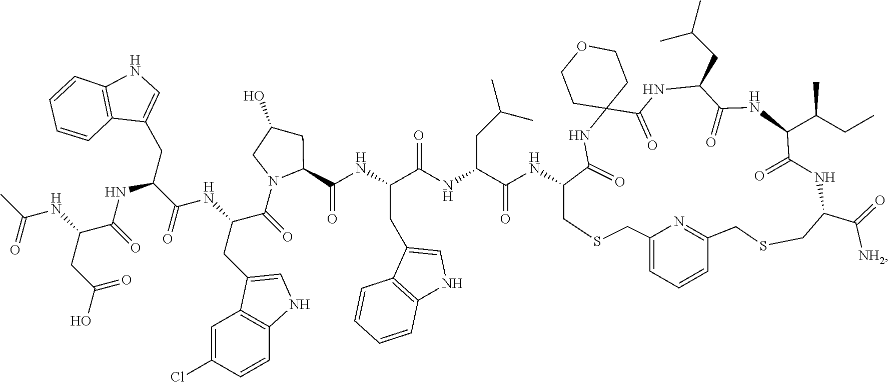 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](Cc2c[nH]c3ccccc23)NC(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CC(=O)O)NC(C)=O)CSCc2cccc(n2)CSC[C@@H](C(N)=O)NC1=O]
compound Ac-Asp-Trp-5Clw-Hyp-Pro-leu-[Cys(2Lut)-Thp-Leu-Ile-Cys]-NH₂ (GIP-398) of the following formula 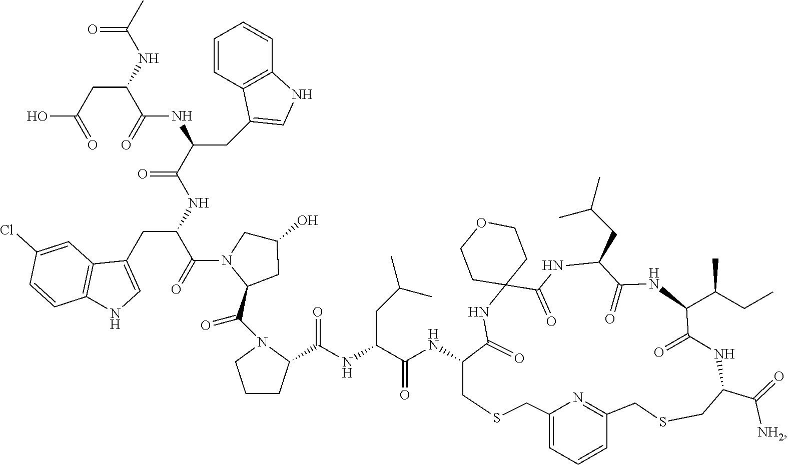 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@@H]2CCCN2C(=O)[C@@H]2C[C@@H](O)CN2C(=O)[C@H](Cc2c[nH]c3ccc(Cl)cc23)NC(=O)[C@H](Cc2c[nH]c3ccccc23)NC(=O)[C@H](CC(=O)O)NC(C)=O)CSCc2cccc(n2)CSC[C@@H](C(N)=O)NC1=O]
compound Ac-Asp-Ocf-5Clw-Hyp-Glu-leu-[Cys(2Lut)-Thp-Leu-Ile-Cys]-lys(DOTA)-NH₂ (GIP-399) of the following formula 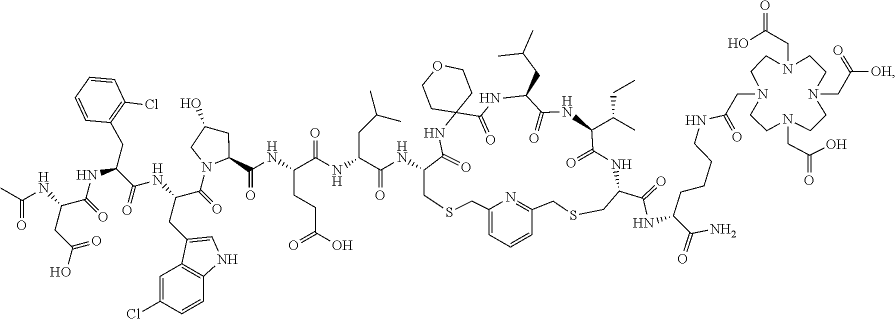 [SMILES: CC[C@H](C)[C@@H]1NC(=O)[C@H](CC(C)C)NC(=O)C2(CCOCC2)NC(=O)[C@@H](NC(=O)[C@@H](CC(C)C)NC(=O)[C@H](CCC(=O)O)NC(=O)[C@@H]2C[C@@H](O)CN2C(=O)[C@H](Cc2c[nH]c3ccc(Cl)cc23)NC(=O)[C@H](Cc2ccccc2Cl)NC(=O)[C@H](CC(=O)O)NC(C)=O)CSCc2cccc(n2)CSC[C@@H](C(=O)N[C@H](CCCCNC(=O)CN2CCN(CC(=O)O)CCN(CC(=O)O)CCN(CC(=O)O)CC2)C(N)=O)NC1=O]
and compound Ac-Asp-Trp-1Ni-Pro-Glu-Hle-[Cys(3MeBn)-Apc(DOTA)-Leu-Ile-Cys]-NH₂ (GIP-400) of the following formula 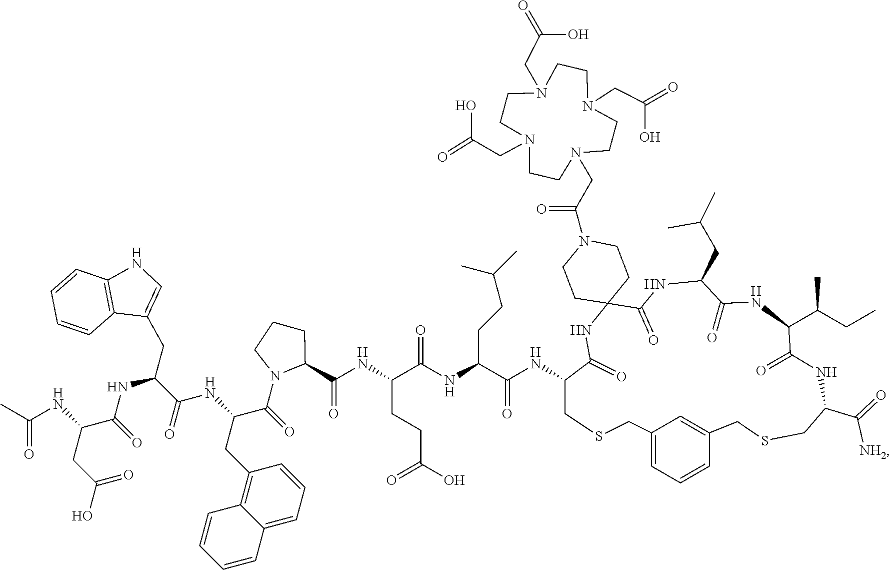 [SMILES: CC[C@H](C)[C@@H]1NC(=O)[C@H](CC(C)C)NC(=O)C2(CCN(C(=O)CN3CCN(CC(=O)O)CCN(CC(=O)O)CCN(CC(=O)O)CC3)CC2)NC(=O)[C@@H](NC(=O)[C@H](CCC(C)C)NC(=O)[C@H](CCC(=O)O)NC(=O)[C@@H]2CCCN2C(=O)[C@H](Cc2cccc3ccccc23)NC(=O)[C@H](Cc2c[nH]c3ccccc23)NC(=O)[C@H](CC(=O)O)NC(C)=O)CSCc2cccc(c2)CSC[C@@H](C(N)=O)NC1=O]
preferably the compound
a) is selected from the group consisting of GIP-001 to GIP-031, GIP-033 to GIP-056, GIP-059 to GIP-224, GIP-226 to GIP-263, GIP-265 to GIP-267, GIP-270, GIP-271, GIP-273, GIP-274, GIP-276, GIP-277, GIP-279, GIP-281, GIP-283, GIP-315, GIP-319, GIP-358, GIP-359, GIP-363 and GIP-370 to GIP-400,
b) preferably is selected from the group consisting of GIP-001 to GIP-031, GIP-033 to GIP-056, GIP-059 to GIP-224, GIP-226 to GIP-263, GIP-265 to GIP-267 and GIP-370 to GIP-386,
c) more preferably is selected from the group consisting of GIP-001 to GIP-031, GIP-033 to GIP-056, GIP-059 to GIP-071 and GIP-370 to GIP-374,
and
d) most preferably is selected from the group consisting of GIP-001 to GIP-024 and GIP-370 to GIP-374.

20. The compound of claim 1, wherein any S-atom, which can be oxidized, preferably any S atom of a thioether group, is present as —S—, —S(O)— or —S(O₂)— or mixture thereof.

21. The compound of claim 1, wherein the compound comprises a radionuclide, wherein the radionuclide is a radionuclide suitable for diagnosis.

22. The compound of claim 21, wherein the radionuclide
is selected from the group comprising, ⁴³Sc, ⁴⁴Sc, ⁵¹Mn, ⁵²Mn, ⁶⁴Cu, ⁶⁷Ga, ⁶⁸Ga, ⁸⁶Y, ⁸⁹Zr, ^{94m}Tc, ^{99m}Tc, ¹¹¹In, ¹⁵²Tb, ¹⁵⁵Tb, ¹⁷⁷Lu, ²⁰¹Tl, ²⁰³Pb, ¹⁸F, ⁷⁶Br, ⁷⁷Br, ¹²³I, ¹²⁴I, and ¹²⁵I,
preferably is selected from the group comprising ⁴³Sc, ⁴⁴Sc, ⁶⁴Cu, ⁶⁷Ga, ⁶⁸Ga, ⁸⁶Y, ⁸⁹Zr, ¹¹¹In, ¹⁵²Tb, ¹⁵⁵Tb, and ²⁰³Pb,
and
more preferably is selected from the group comprising ⁶⁴Cu, ⁶⁸Ga, ¹¹¹In and ²⁰³Pb.

23. The compound of claim 1, wherein the compound comprises a radionuclide, wherein the radionuclide is a radionuclide suitable for therapy.

24. The compound of claim 23, wherein the radionuclide is selected from the group comprising ⁴⁷Sc, ⁶⁷Cu, ⁸⁹Sr, ⁹⁰Y, ¹¹¹In, ¹⁵³Sm, ¹⁴⁹Tb, ¹⁶¹Tb, ¹⁷⁷Lu, ¹⁸⁶Re, ¹⁸⁸Re, ²¹²Pb, ²¹³Bi, ²²³Ra, ²²⁵Ac, ²²⁶Th, ²²⁷Th, ¹³¹I, and ²¹¹At, preferably is selected from the group comprising ⁴⁷Sc, ⁶⁷Cu, ⁹⁰Y, ¹⁷⁷Lu, ²¹²Pb, ²¹³Bi, ²²⁵Ac, and ²²⁷Th,
and more preferably is selected from the group comprising ⁹⁰Y, ¹⁷⁷Lu, ²¹²Pb, ²²⁵Ac, and ²²⁷Th.

25. The compound of claim 1, for use in a method for diagnosing a disease.

26. The compound of claim 1, for use in a method for the treatment of a disease.

27. The compound of claim 1, for use in a method for the identification of a subject, wherein the subject is likely to respond or likely not to respond to a treatment of a disease, wherein the method for the identification of a subject comprises carrying out a method of diagnosing a disease using a compound of any one of claims 1 to 24.

28. The compound of claim 1, for use in a method for the selection of a subject from a group of subjects, wherein the subject is likely to respond or likely not to respond to a treatment of a disease, wherein the method for the selection of a subject from a group of subjects comprises carrying out a method of diagnosing a disease using a compound of any one of claims 1 to 24.

29. The compound of claim 1, for use in a method for the stratification of a group of subjects into subjects which are likely to respond to a treatment of a disease, and into subjects which are not likely to respond to a treatment of a disease, wherein the method for the stratification of a group of subjects comprises carrying out a method of diagnosing a disease using a compound of any one of claims 1 to 24.

30. The compound for use of claim 25, wherein the disease is cancer.

31. The compound for use of claim 30, wherein the cancer is expressing Gastric Inhibitory Peptide Receptor (GIPR), preferably the cancer is overexpressing GIPR.

32. The compound for use of claim 30, wherein the cancer is a neuroendocrine cancer or a neuroendocrine neoplasm.

33. The compound for use of claim 30, wherein the cancer is selected from the group consisting of medullary thyroid cancer, gastrointestinal neuroendocrine neoplasms, ileal neuroendocrine neoplasms, pancreatic neuroendocrine neoplasms, nonfunctioning neuroendocrine neoplasms, Insulinomas, Gastrinomas, Glucagonomas, VIPomas, Somatostatinoma, ACTHoma, lung neuroendocrine neoplasms, typical carcinoid tumors, atypical carcinoid tumors, small cell carcinoma of the lung, large cell carcinoma of the lung, thymic neuroendocrine tumors, Merkel cell carcinoma, Pheochromocytoma of the adrenal gland, adrenal cancer, parathyroid cancer, paraganglioma, pituitary gland tumors, neuroendocrine tumors of the ovaries and neuroendocrine tumors of the testicles.

34. The compound for use of claim 25, wherein the disease is a metabolic disease.

35. The compound for use of claim 34, wherein a diseased cell and/or a diseased tissue, preferably a diseased cell and/or a diseased tissue of the metabolic disease expresses GIPR.

36. The compound for use of claim 35, wherein the diseased cell and/or the diseased tissue overexpresses GIPR.

37. The compound for use of claim 35, wherein the diseased cell is a pancreatic cell.

38. The compound for use of claim 34, wherein the metabolic disease is selected from the group consisting of type 2 diabetes, type 1 diabetes, metabolic syndrome, insulin resistance, dyslipidemia, impaired fasting glucose, and impaired glucose tolerance.

39. A composition comprising a compound of claim 1 and a pharmaceutically acceptable excipient.

40. The composition of claim 39, wherein the composition is a pharmaceutical composition.

41. A kit comprising a compound of claim 1 and one or more optional excipient(s) and optionally one or more device(s).

42. The kit of claim 41, wherein the device(s) is/are selected from the group comprising a labeling device, a purification device, a handling device, a radioprotection device, an analytical device or an administration device.


SEQUENCE LISTING (395 sequences)

SEQ ID NO: 1 - protein, 466 aa, Homo sapiens
MTTSPILQLLLRLSLCGLLLQRAETGSKGQTAGELYQRWERYRRECQETLAAAEPPSGLACNGSFDMYVCWDYAAPNATARASCPWYLPWHHHVAAGFVLRQCGSDGQWGLWRDHTQCENPEKNEAFLDQRLILERLQVMYTVGYSLSLATLLLALLILSLFRRLHCTRNYIHINLFTSFMLRAAAILSRDRLLPRPGPYLGDQALALWNQALAACRTAQIVTQYCVGANYTWLLVEGVYLHSLLVLVGGSEEGHFRYYLLLGWGAPALFVIPWVIVRYLYENTQCWERNEVKAIWWIIRTPILMTILINFLIFIRILGILLSKLRTRQMRCRDYRLRLARSTLTLVPLLGVHEVVFAPVTEEQARGALRFAKLGFEIFLSSFQGFLVSVLYCFINKEVQSEIRRGWHHCRLRRSLGEEQRQLPERAFRALPSGSGPGEVPTSRGLSSGTLPGPGNEASRELESYC

SEQ ID NO: 2 - protein, 42 aa, Homo sapiens
YAQGTFISDYSIAMDKIHQQNFVNWLLAQKGKKNDQKHNITQ

SEQ ID NO: 3 - protein, 11 aa, synthetic construct
  SITE 1: note = X is D, E or succinyl
  SITE 2: note = X is W or 5-hydroxy-L-tryptophane
  SITE 3: note = X is 5-chloro-L-tryptophan or 5-bromo-L-tryptophan
  SITE 4: note = X is (2S,4R)-4-amino-1-pyrrolidin-2-carboxylic acid, 4-(R)-hydroxy-L-proline or P
  SITE 6: note = X is D-leucine or L
  SITE 8: note = X is 4-aminopiperidine-4-carboxylic acid DOTA conjugate or 4-amino-tetrahydropyran-4-carboxylic acid
  CROSSLNK 7..11: note = cysteine cyseine linked through 3,5-lutidylidene
XXXXEXCXLIC

SEQ ID NO: 4 - protein, 11 aa, synthetic construct
  SITE 3: note = X is 5-chloro-L-tryptophan
  SITE 4: note = X is (2S,4R)-4-amino-1-pyrrolidin-2-carboxylic acid or 4-(R)-hydroxy-L-proline
  SITE 8: note = X is 4-aminopiperidine-4-carboxylic acid DOTA conjugate
  CROSSLNK 7..11: note = cysteine cysteine conencted through 3,5-lutidylidene
DTXXEXCXLIC

SEQ ID NO: 5 - protein, 11 aa, synthetic construct
  MOD_RES 1: note = 1-amino-1-deoxy-D-glucitol attached through glutaric acid to N-terminus
  SITE 3: note = X is 5-chloro-L-tryptophan
  SITE 4: note = X is (2S,4R)-4-amino-1-pyrrolidin-2-carboxylic acid
  SITE 6: note = X is D-leucine
  SITE 8: note = X is 4-aminopiperidine-4-carboxylic acid DOTA conjugate
  MOD_RES 11: note = D-glutamic acid attached through 4-amino-1-carboxymethylpiperidine to C-terminus with 1-amino-1-deoxy-D-glucitol being attached to D-glutamic acid
  CROSSLNK 7..11: note = cysteine cysteine connected through 2,6-lutidylidene
DWXXEXCXLIC

SEQ ID NO: 6 - protein, 11 aa, synthetic construct
  MOD_RES 1: note = 1-amino-1-deoxy-D-glucitol attached through glutaric acid to N-terminus
  SITE 3: note = X is 5-chloro-L-tryptophan
  SITE 4: note = X is 4-(R)-hydroxy-L-proline
  SITE 6: note = X is D-leucine
  SITE 8: note = X is 4-aminopiperidine-4-carboxylic acid DOTA conjugate
  MOD_RES 11: note = D-glutamic acid attached through 4-amino-1-carboxymethylpiperidine to C-terminus with 1-amino-1-deoxy-D-glucitol being attached to D-glutamic acid
  CROSSLNK 7..11: note = cysteine cysteine connected through 2,6-lutidylidene
DWXXEXCXLIC

SEQ ID NO: 7 - protein, 11 aa, synthetic construct
  MOD_RES 1: note = DOTA attached to D-glutamic acid bearing 1-amino-1-deoxy-D-glucitol with the D-glutamic acid being attached to D through 4-amino-1-carboxymethylpiperidine
  SITE 3: note = X is 5-chloro-L-tryptophan
  SITE 4: note = X is 4-(R)-hydroxy-L-proline
  SITE 6: note = X is D-leucine
  SITE 8: note = X is 4-aminopiperidine-4-carboxylic acid to which 1-amino-1-deoxy-D-glucitol is attached through glutaric acid
  MOD_RES 11: note = D-glutamic acid attached through 4-amino-1-carboxymethylpiperidine to C-terminus with 1-amino-1-deoxy-D-glucitol being attached to D-glutamic acid
  CROSSLNK 7..11: note = note = cysteine cysteine connected through 2,6-lutidylidene
DWXXEXCXLIC

SEQ ID NO: 8 - protein, 11 aa, synthetic construct
  MOD_RES 1: note = 1-amino-1-deoxy-D-glucitol attached through glutaric acid to N-terminus
  SITE 3: note = X is 5-chloro-L-tryptophan
  SITE 4: note = X is 4-(R)-hydroxy-L-proline
  SITE 6: note = X is D-leucine
  MOD_RES 11: note = D-glutamic acid attached through 4-amino-1-carboxymethylpiperidine to C-terminus with 1-amino-1-deoxy-D-glucitol being and DOTA bearing lysine attached to D-glutamic acid
  CROSSLNK 7..11: note = cysteine cysteine connected through 2,6-lutidylidene
DWXXEXCXLIC

SEQ ID NO: 9 - protein, 11 aa, synthetic construct
  MOD_RES 1: note = DOTA attached to D-glutamic acid bearing 1-amino-1-deoxy-D-glucitol, whereby the D-glutamic acid is attached through 4-amino-1-carboxymethylpiperidine to N-terminus of D-glutamic acid
  SITE 3: note = X is 5-chloro-L-tryptophan
  SITE 4: note = X is 4-(R)-hydroxy-L-proline
  SITE 6: note = X is D-leucine
  SITE 8: note = X is 4-amino-tetrahydropyran-4-carboxylic acid
  MOD_RES 11: note = D-glutamic acid attached through 4-amino-1-carboxymethylpiperidine to C-terminus with 1-amino-1-deoxy-D-glucitol being attached to D-glutamic acid
  CROSSLNK 7..11: note = cysteine cysteine connected through 2,6-lutidylidene
DWXXEXCXLIC

SEQ ID NO: 10 - protein, 11 aa, synthetic construct
  MOD_RES 1: note = 1-amino-1-deoxy-D-glucitol bearing glutaric acid attached to D through 4-amino-1-carboxymethylpiperidine
  SITE 3: note = X is 5-chloro-L-tryptophan
  SITE 4: note = X is (2S,4R)-4-amino-1-pyrrolidin-2-carboxylic acid
  SITE 6: note = D-leucine
  SITE 8: note = X is 4-aminopiperidine-4-carboxylic acid DOTA conjugate
  MOD_RES 11: note = D-glutamic acid attached through 4-amino-1-carboxymethylpiperidine to C-terminus with 1-amino-1-deoxy-D-glucitol being attached to D-glutamic acid
  CROSSLNK 7..11: note = cysteine cysteine connected through 2,6-lutidylidene
DWXXEXCXLIC

SEQ ID NO: 11 - protein, 12 aa, synthetic construct
  MOD_RES 11: note = Glutaric acid bearing 1-amino-1-deoxy-D-glucitol is attached to D through 4-amino-1-carboxymethylpiperidine
  SITE 3: note = X is 5-chloro-L-tryptophan
  SITE 4: note = X is (2S,4R)-4-amino-1-pyrrolidin-2-carboxylic acid
  SITE 6: note = X is D-leucine
  SITE 8: note = X is 4-aminopiperidine-4-carboxylic acid (glu(DOTA/AGLU)) conjugate
  MOD_RES 11: note = D-glutamic acid attached through 4-amino-1-carboxymethylpiperidine to C-terminus with 1-amino-1-deoxy-D-glucitol being attached to D-glutamic acid
  CROSSLNK 7..11: note = cysteine cysteine connected through 2,6-lutidylidene
DWXXEXCXXLIC

SEQ ID NO: 12 - protein, 11 aa, synthetic construct
  MOD_RES 1: note = Glutaric acid bearing 1-amino-1-deoxy-D-glucitol is attached to D through 4-amino-1-carboxymethylpiperidine
  SITE 3: note = X is 5-chloro-L-tryptophan
  SITE 4: note = X is (2S,4R)-4-amino-1-pyrrolidin-2-carboxylic acid
  SITE 6: note = X is D-leucine
  SITE 8: note = X is 4-aminopiperidine-4-carboxylic acid (glu(DOTA/AGLU)) conjugate
  MOD_RES 11: note = D-glutamic acid attached through 4-amino-1-carboxymethylpiperidine to C-terminus with 1-amino-1-deoxy-D-glucitol being attached to D-glutamic acid
  CROSSLNK 7..11: note = cysteine cysteine connected through 2,6-lutidylidene
DWXXEXCXLIC

SEQ ID NO: 13 - protein, 11 aa, synthetic construct
  MOD_RES 1: note = DOTA attached to D-glutamic acid bearing 1-amino-1-deoxy-D-glucitol, whereby the D-glutamic acid is attached through 8-amino-3,6-dioxaoctanoic acid to N-terminus of D
  SITE 3: note = X is 5-chloro-L-tryptophan
  SITE 4: note = X is (2S,4R)-4-amino-1-pyrrolidin-2-carboxylic acid
  SITE 6: note = X is D-leucine
  SITE 8: note = X is 4-aminopiperidine-4-carboxylic acid to which 1-amino-1-deoxy-D-glucitol is attached through glutaric acid
  MOD_RES 11: note = D-glutamic acid attached through 4-amino-1-carboxymethylpiperidine to C-terminus with 1-amino-1-deoxy-D-glucitol being attached to D-glutamic acid
  CROSSLNK 7..11: note = cysteine cysteine connected through 2,6-lutidylidene
DWXXEXCXLIC

SEQ ID NO: 14 - protein, 11 aa, synthetic construct
  MOD_RES 1: note = 1-amino-1-deoxy-D-glucitol attached through glutaric acid to N-terminus
  SITE 3: note = X is 5-chloro-L-tryptophan
  SITE 4: note = 4-(R)-hydroxy-L-proline
  SITE 6: note = X is D-leucine
  SITE 8: note = X is 4-aminopiperidine-4-carboxylic acid to which 1-amino-1-deoxy-D-glucitol is attached through glutaric acid
  MOD_RES 11: note = DOTA bearing K attached to C through 4-amino-1-carboxymethylpiperidine
  CROSSLNK 7..11: note = cysteine cysteine connected through 2,6-lutidylidene
DWXXEXCXLIC

SEQ ID NO: 15 - protein, 11 aa, synthetic construct
  MOD_RES 1: note = Glutaric acid bearing 1-amino-1-deoxy-D-glucitol is attached to D through 4-amino-1-carboxymethylpiperidine
  SITE 3: note = X is 5-chloro-L-tryptophan
  SITE 4: note = X is 4-(R)-hydroxy-L-proline
  SITE 8: note = X is 4-aminopiperidine-4-carboxylic acid DOTA conjugate
  MOD_RES 11: note = D-glutamic acid attached through 4-amino-1-carboxymethylpiperidine to C-terminus with 1-amino-1-deoxy-D-glucitol being attached to D-glutamic acid
  CROSSLNK 7..11: note = cysteine cysteine connected through 2,6-lutidylidene
DWXXEXCXLIC

SEQ ID NO: 16 - protein, 11 aa, synthetic construct
  MOD_RES 1: note = DOTA attached to D through 4-amino-1-carboxymethylpiperidine
  SITE 3: note = X is 5-chloro-L-tryptophan
  SITE 4: note = 4-(R)-hydroxy-L-proline
  SITE 6: note = X is D-leucine
  SITE 8: note = X is 4-aminopiperidine-4-carboxylic acid to which 1-amino-1-deoxy-D-glucitol is attached through glutaric acid
  MOD_RES 11: note = D-glutamic acid attached through 4-amino-1-carboxymethylpiperidine to C-terminus with 1-amino-1-deoxy-D-glucitol being attached to D-glutamic acid
  CROSSLNK 7..11: note = cysteine cysteine connected through 2,6-lutidylidene
DWXXEXCYLIC

SEQ ID NO: 17 - protein, 11 aa, synthetic construct
  MOD_RES 1: note = Glutaric acid bearing 1-amino-1-deoxy-D-glucitol is attached to D through 4-amino-1-carboxymethylpiperidine
  SITE 3: note = X is 5-chloro-L-tryptophan
  SITE 4: note = X is (2S,4R)-4-amino-1-pyrrolidin-2-carboxylic acid
  SITE 6: note = X is D-leucine
  SITE 8: note = 4-aminopiperidine-4-carboxylic acid DOTA conjugate
  MOD_RES 11: note = D-glutamic acid attached through 4-amino-1-carboxymethylpiperidine to C-terminus with 1-amino-1-deoxy-D-glucitol being attached to D-glutamic acid
  CROSSLNK 7..11: note = cysteine cysteine connected through 2,6-lutidylidene
DWXXEXCXLIX

SEQ ID NO: 18 - protein, 11 aa, synthetic construct
  MOD_RES 1: note = Glutaric acid bearing 1-amino-1-deoxy-D-glucitol is attached to D through 4-amino-1-carboxymethylpiperidine
  SITE 3: note = X is 5-bromo-L-tryptophan
  SITE 4: note = X is (2S,4R)-4-amino-1-pyrrolidin-2-carboxylic acid
  SITE 6: note = X is D-leucine
  SITE 8: note = X is 4-aminopiperidine-4-carboxylic acid DOTA conjugate
  MOD_RES 11: note = D-glutamic acid attached through 4-amino-1-carboxymethylpiperidine to C-terminus with 1-amino-1-deoxy-D-glucitol being attached to D-glutamic acid
  CROSSLNK 7..11: note = cysteine cysteine connected through 2,6-lutidylidene
DWXXEXCXLIC

SEQ ID NO: 19 - protein, 11 aa, synthetic construct
  MOD_RES 1: note = 1-amino-1-deoxy-D-glucitol attached through glutaric acid to D
  SITE 3: note = X is 5Brw = 5-bromo-L-tryptophan
  SITE 4: note = X is (2S,4R)-4-amino-1-pyrrolidin-2-carboxylic acid
  SITE 6: note = X is D-leucine
  SITE 8: note = X is 4-aminopiperidine-4-carboxylic acid DOTA conjugate
  MOD_RES 11: note = D-glutamic acid attached through 4-amino-1-carboxymethylpiperidine to C-terminus with 1-amino-1-deoxy-D-glucitol being attached to D-glutamic acid
  CROSSLNK 7..11: note = cysteine cysteine connected through 2,6-lutidylidene
DWXXEXCXLIC

SEQ ID NO: 20 - protein, 11 aa, synthetic construct
  SITE 1: note = X is L-homoserine
  SITE 3: note = X is 5-bromo-L-tryptophan
  SITE 4: note = X is (2S,4R)-4-amino-1-pyrrolidin-2-carboxylic acid
  SITE 6: note = X is D-leucine
  SITE 8: note = X is 4-aminopiperidine-4-carboxylic acid DOTA conjugate
  MOD_RES 1: note = 1-amino-1-deoxy-D-glucitol attached through glutaric acid to L-homoserine
  MOD_RES 11: note = D-glutamic acid attached through 4-amino-1-carboxymethylpiperidine to C-terminus with 1-amino-1-deoxy-D-glucitol being attached to D-glutamic acid
  CROSSLNK 7..11: note = cysteine cysteine connected through 2,6-lutidylidene
XWXXEXCXLIC

SEQ ID NO: 21 - protein, 11 aa, synthetic construct
  MOD_RES 1: note = DOTA attached to D-glutamic acid bearing 1-amino-1-deoxy-D-glucitol, whereby the D-glutamic acid is attached D
  SITE 3: note = X is 5-chloro-L-tryptophan
  SITE 4: note = X is (2S,4R)-4-amino-1-pyrrolidin-2-carboxylic acid
  SITE 6: note = X is D-leucine
  SITE 8: note = X is 4-amino-tetrahydropyran-4-carboxylic acid
  MOD_RES 11: note = D-glutamic acid attached through 4-amino-1-carboxymethylpiperidine to C-terminus with 1-amino-1-deoxy-D-glucitol being attached to D-glutamic acid
  CROSSLNK 7..11: note = cysteine cysteine connected through 2,6-lutidylidene
DWXXEXCXLIC

SEQ ID NO: 22 - protein, 11 aa, synthetic construct
  MOD_RES 1: note = DOTA attached through 8-amino-3,6-dioxaoctanoic acid to D-glutamic acid bearing 1-amino-1-deoxy-D-glucitol, whereby the D-glutamic acid is attached to D
  SITE 3: note = X is 5-chloro-L-tryptophan
  SITE 4: note = X is (2S,4R)-4-amino-1-pyrrolidin-2-carboxylic acid
  SITE 6: note = X is D-leucine
  SITE 8: note = X is 4-amino-tetrahydropyran-4-carboxylic acid
  MOD_RES 11: note = D-glutamic acid attached through 4-amino-1-carboxymethylpiperidine to C-terminus with 1-amino-1-deoxy-D-glucitol being attached to D-glutamic acid
  CROSSLNK 7..11: note = cysteine cysteine connected through 2,6-lutidylidene
DWXXEXCXLIC

SEQ ID NO: 23 - protein, 11 aa, synthetic construct
  MOD_RES 1: note = DOTA attached to D-glutamic acid bearing 1-amino-1-deoxy-D-glucitol, whereby the D-glutamic acid is attached through 21-amino-4,7,10,13,16,19-hexaoxaheneicosanoic acid to D
  SITE 3: note = X is 5-chloro-L-tryptophan
  SITE 4: note = X is (2S,4R)-4-amino-1-pyrrolidin-2-carboxylic acid
  SITE 6: note = X is D-leucine
  SITE 8: note = X is 4-amino-tetrahydropyran-4-carboxylic acid
  MOD_RES 11: note = D-glutamic acid attached through 4-amino-1-carboxymethylpiperidine to C-terminus with 1-amino-1-deoxy-D-glucitol being attached to D-glutamic acid
  CROSSLNK 7..11: note = cysteine cysteine connected through 2,6-lutidylidene
DWXXEXCXLIC

SEQ ID NO: 24 - protein, 11 aa, synthetic construct
  MOD_RES 1: note = DOTA attached to D-glutamic acid bearing 1-amino-1-deoxy-D-glucitol, whereby the D-glutamic acid is attached through 8-amino-3,6-dioxaoctanoic acid to N-terminus of D
  SITE 3: note = X is 5-chloro-L-tryptophan
  SITE 4: note = X is (2S,4R)-4-amino-1-pyrrolidin-2-carboxylic acid
  SITE 6: note = X is D-leucine
  SITE 8: note = X is 4-amino-tetrahydropyran-4-carboxylic acid
  MOD_RES 11: note = D-glutamic acid attached through 4-amino-1-carboxymethylpiperidine to C-terminus with 1-amino-1-deoxy-D-glucitol being attached to D-glutamic acid
  CROSSLNK 7..11: note = cysteine cysteine connected through 2,6-lutidylidene
DWXXEXCXLIC

SEQ ID NO: 25 - protein, 10 aa, synthetic construct
  MOD_RES 1: note = HO-succinyl attached to W
  SITE 2: note = X is 5-chloro-L-tryptophan
  SITE 3: note = X is 4-(R)-hydroxy-L-proline
  SITE 5: note = X is D-leucine
  SITE 7: note = X is 4-amino-tetrahydropyran-4-carboxylic acid
  MOD_RES 10: note = D-glutamic acid attached through 4-amino-1-carboxymethylpiperidine to C-terminus, wherein to D-glutamic acid 1-amino-1-deoxy-D-glucitol and DOTA bearing K are attached
  CROSSLNK 6..10: note = cysteine cysteine connected through 2,6-lutidylidene
WXXEXCXLIC

SEQ ID NO: 26 - protein, 10 aa, synthetic construct
  MOD_RES 1: note = HO-succinyl attached to W
  SITE 2: note = X is 5-chloro-L-tryptophan
  SITE 3: note = X is (2S,4R)-4-amino-1-pyrrolidin-2-carboxylic acid
  SITE 5: note = X is D-leucine
  SITE 7: note = X is 4-aminopiperidine-4-carboxylic acid
  MOD_RES 10: note = D-glutamic acid attached through 4-amino-1-carboxymethylpiperidine to C-terminus, wherein to D-glutamic acid 1-amino-1-deoxy-D-glucitol and DOTA bearing K are attached
  CROSSLNK 6..10: note = cysteine cysteine connected through 2,6-lutidylidene
WXXEXCXLIC

SEQ ID NO: 27 - protein, 10 aa, synthetic construct
  MOD_RES 1: note = HO-succinyl attached to W
  SITE 2: note = X is 5-chloro-L-tryptophan
  SITE 3: note = X is (2S,4R)-4-amino-1-pyrrolidin-2-carboxylic acid
  SITE 5: note = X is D-leucine
  SITE 7: note = X is 4-aminopiperidine-4-carboxylic acid DOTA conjugate
  MOD_RES 10: note = D-glutamic acid attached through 4-amino-1-carboxymethylpiperidine to C-terminus with 1-amino-1-deoxy-D-glucitol being attached to D-glutamic acid
  CROSSLNK 6..10: note = cysteine cysteine connected through 2,6-lutidylidene
WXXEXCXLIC

SEQ ID NO: 28 - protein, 11 aa, synthetic construct
  MOD_RES 1: note = 1-amino-1-deoxy-D-glucitol attached through glutaric acid to D
  SITE 3: note = X is 5-chloro-L-tryptophan
  SITE 4: note = X is (2S,4R)-4-amino-1-pyrrolidin-2-carboxylic acid
  SITE 6: note = X is D-leucine
  SITE 8: note = X is D-lysine with DOTA attached
  MOD_RES 11: note = D-glutamic acid attached through 4-amino-1-carboxymethylpiperidine to C-terminus with 1-amino-1-deoxy-D-glucitol being attached to D-glutamic acid
  CROSSLNK 7..11: note = cysteine cysteine connected through 2,6-lutidylidene
DWXXEXCXLIC

SEQ ID NO: 29 - protein, 11 aa, synthetic construct
  MOD_RES 1: note = 1-amino-1-deoxy-D-glucitol attached through glutaric acid to D
  SITE 3: note = X is 5-chloro-L-tryptophan
  SITE 4: note = X is (2S,4R)-4-amino-1-pyrrolidin-2-carboxylic acid
  SITE 6: note = X is D-leucine
  SITE 8: note = X is D-lysine with DOTA attached
  MOD_RES 11: note = D-glutamic acid attached through 4-amino-1-carboxymethylpiperidine to C-terminus with 1-amino-1-deoxy-D-glucitol being attached to D-glutamic acid
  CROSSLNK 7..11: note = cysteine cysteine connected through 2,6-lutidylidene
DWXXEXCXLIC

SEQ ID NO: 30 - protein, 11 aa, synthetic construct
  MOD_RES 1: note = 1-amino-1-deoxy-D-glucitol attached through glutaric acid to D
  SITE 3: note = X is 5-chloro-L-tryptophan
  SITE 4: note = X is (2S,4R)-4-amino-1-pyrrolidin-2-carboxylic acid
  SITE 6: note = X is D-leucine
  SITE 8: note = X is (S)-2,4-Diaminobutyric acid with DOTA attached
  MOD_RES 11: note = D-glutamic acid attached through 4-amino-1-carboxymethylpiperidine to C-terminus with 1-amino-1-deoxy-D-glucitol being attached to D-glutamic acid
  CROSSLNK 7..11: note = cysteine cysteine connected through 2,6-lutidylidene
DWXXEXCXLIC

SEQ ID NO: 31 - protein, 11 aa, synthetic construct
  MOD_RES 1: note = 1-amino-1-deoxy-D-glucitol attached through glutaric acid to D
  SITE 3: note = X is 5-chloro-L-tryptophan
  SITE 4: note = X is (2S,4R)-4-amino-1-pyrrolidin-2-carboxylic acid
  SITE 6: note = X is D-leucine
  SITE 8: note = X is (R)-2,3-Diaminopropionic acid with DOTA attached
  MOD_RES 11: note = D-glutamic acid attached through 4-amino-1-carboxymethylpiperidine to C-terminus with 1-amino-1-deoxy-D-glucitol being attached to D-glutamic acid
  CROSSLNK 7..11: note = cysteine cysteine connected through 2,6-lutidylidene
DWXXEXCXLIC

SEQ ID NO: 32 - protein, 11 aa, synthetic construct
  MOD_RES 1: note = 1-amino-1-deoxy-D-glucitol attached through glutaric acid to D
  SITE 3: note = X is 5-chloro-L-tryptophan
  SITE 4: note = X is (2S,4R)-4-amino-1-pyrrolidin-2-carboxylic acid
  SITE 6: note = X is D-leucine
  SITE 8: note = X is 4-aminopiperidine-4-carboxylic acid with NODAGA attached
  MOD_RES 11: note = D-glutamic acid attached through 4-amino-1-carboxymethylpiperidine to C-terminus with 1-amino-1-deoxy-D-glucitol being attached to D-glutamic acid
  CROSSLNK 7..11: note = cysteine cysteine connected through 2,6-lutidylidene
DWXXEXCXLIC

SEQ ID NO: 33 - protein, 11 aa, synthetic construct
  MOD_RES 1: note = 1-amino-1-deoxy-D-glucitol attached through glutaric acid to D
  SITE 3: note = X is 5-chloro-L-tryptophan
  SITE 4: note = X is (2S,4R)-4-amino-1-pyrrolidin-2-carboxylic acid
  SITE 6: note = X is D-leucine
  SITE 8: note = X is 4-aminopiperidine-4-carboxylic acid with NOPO attached
  MOD_RES 11: note = D-glutamic acid attached through 4-amino-1-carboxymethylpiperidine to C-terminus with 1-amino-1-deoxy-D-glucitol being attached to D-glutamic acid
  CROSSLNK 7..11: note = cysteine cysteine connected through 2,6-lutidylidene
DWXXEXCXLIC

SEQ ID NO: 34 - protein, 11 aa, synthetic construct
  MOD_RES 1: note = 1-amino-1-deoxy-D-glucitol attached through glutaric acid to D
  SITE 3: note = X is 5-chloro-L-tryptophan
  SITE 4: note = X is (2S,4R)-4-amino-1-pyrrolidin-2-carboxylic acid
  SITE 6: note = X is D-leucine
  SITE 8: note = X is 4-aminopiperidine-4-carboxylic acid with N4Ac attached
  MOD_RES 11: note = D-glutamic acid attached through 4-amino-1-carboxymethylpiperidine to C-terminus with 1-amino-1-deoxy-D-glucitol being attached to D-glutamic acid
  CROSSLNK 7..11: note = cysteine cysteine connected through 2,6-lutidylidene
DWXXEXCXLIC

SEQ ID NO: 35 - protein, 11 aa, synthetic construct
  MOD_RES 1: note = 1-amino-1-deoxy-D-glucitol attached through glutaric acid to D
  SITE 3: note = 5-chloro-L-tryptophan
  SITE 4: note = X is (2S,4R)-4-amino-1-pyrrolidin-2-carboxylic acid
  SITE 6: note = X is D-leucine
  SITE 8: note = X is 4-aminopiperidine-4-carboxylic acid with NODAGA attached through glycine
  MOD_RES 11: note = D-glutamic acid attached through 4-amino-1-carboxymethylpiperidine to C-terminus with 1-amino-1-deoxy-D-glucitol being attached to D-glutamic acid
  CROSSLNK 7..11: note = cysteine cysteine connected through 2,6-lutidylidene
DWXXEXCXLIC

SEQ ID NO: 36 - protein, 11 aa, synthetic construct
  MOD_RES 1: note = Glutaric acid bearing 1-amino-1-deoxy-D-glucitol is attached to D through 4-amino-1-carboxymethylpiperidine
  SITE 3: note = X is 5-chloro-L-tryptophan
  SITE 4: note = 4-(R)-hydroxy-L-proline
  SITE 6: note = X is D-leucine
  SITE 8: note = X is 4-aminopiperidine-4-carboxylic acid DOTA conjugate
  MOD_RES 11: note = 1-amino-1-deoxy-D-glucitol attached to D-glutamic acid
  CROSSLNK 7..11: note = cysteine cysteine connected through 2,6-lutidylidene
DWXXEXCXLIC

SEQ ID NO: 37 - protein, 11 aa, synthetic construct
  MOD_RES 1: note = 1-amino-1-deoxy-D-glucitol attached through glutaric acid to L-homoserine
  SITE 1: note = X is L-homoserine
  SITE 3: note = X is 5-chloro-L-tryptophan
  SITE 4: note = X is (2S,4R)-4-amino-1-pyrrolidin-2-carboxylic acid
  SITE 6: note = X is D-leucine
  SITE 8: note = 4-aminopiperidine-4-carboxylic acid DOTA conjugate
  MOD_RES 11: note = D-glutamic acid attached through 4-amino-1-carboxymethylpiperidine to C-terminus with 1-amino-1-deoxy-D-glucitol being attached to D-glutamic acid
  CROSSLNK 7..11: note = cysteine cysteine connected through 2,6-lutidylidene
XWXXEXCXLIC

SEQ ID NO: 38 - protein, 11 aa, synthetic construct
  MOD_RES 1: note = 1-amino-1-deoxy-D-glucitol attached through glutaric acid to D
  SITE 3: note = X is 5-chloro-L-tryptophan
  SITE 4: note = X is (2S,4R)-4-amino-1-pyrrolidin-2-carboxylic acid
  SITE 8: note = X is 4-aminopiperidine-4-carboxylic acid with DOTA attached
  MOD_RES 11: note = D-glutamic acid attached through 4-amino-1-carboxymethylpiperidine to C-terminus with 1-amino-1-deoxy-D-glucitol being attached to D-glutamic acid
  CROSSLNK 7..11: note = cysteine cysteine connected through 2,6-lutidylidene
DWXXELCXLIC

SEQ ID NO: 39 - protein, 11 aa, synthetic construct
  MOD_RES 1: note = DOTA attached to D-glutamic acid bearing 1-amino-1-deoxy-D-glucitol, whereby the D-glutamic acid is attached D
  SITE 3: note = X is 5-chloro-L-tryptophan
  SITE 4: note = X is 4-(R)-hydroxy-L-proline
  SITE 6: note = X is D-leucine
  SITE 8: note = X is 4-amino-tetrahydropyran-4-carboxylic acid
  MOD_RES 11: note = D-glutamic acid attached through 4-amino-1-carboxymethylpiperidine to C-terminus with 1-amino-1-deoxy-D-glucitol being attached to D-glutamic acid
  CROSSLNK 7..11: note = cysteine cysteine connected through 2,6-lutidylidene
DWXXEXCXLIC

SEQ ID NO: 40 - protein, 11 aa, synthetic construct
  MOD_RES 1: note = DOTA attached to D-glutamic acid through 21-amino-4,7,10,13,16,19-hexaoxaheneicosanoic acid, whereby D-glutamic acid bearing 1-amino-1-deoxy-D-glucitol is attached to D
  SITE 3: note = X is 5-chloro-L-tryptophan
  SITE 4: note = X is (2S,4R)-4-amino-1-pyrrolidin-2-carboxylic acid
  SITE 6: note = X is D-leucine
  SITE 8: note = X is 4-amino-tetrahydropyran-4-carboxylic acid
  MOD_RES 11: note = D-glutamic acid attached through 4-amino-1-carboxymethylpiperidine to C-terminus with 1-amino-1-deoxy-D-glucitol being attached to D-glutamic acid
  CROSSLNK 7..11: note = cysteine cysteine connected through 2,6-lutidylidene
DWXXEXCXLIC

SEQ ID NO: 41 - protein, 11 aa, synthetic construct
  MOD_RES 1: note = 1-amino-1-deoxy-D-glucitol attached through glutaric acid to D
  SITE 3: note = X is 5-chloro-L-tryptophan
  SITE 4: note = X is (2S,4R)-4-amino-1-pyrrolidin-2-carboxylic acid
  SITE 6: note = X is D-leucine
  SITE 8: note = X is 4-aminopiperidine-4-carboxylic acid DOTA conjugate
  MOD_RES 11: note = D-glutamic acid attached through 4-amino-1-carboxymethylpiperidine to C-terminus with 1-amino-1-deoxy-D-glucitol being attached to D-glutamic acid
  CROSSLNK 7..11: note = cysteine cysteine connected through 2,6-lutidylidene
DWXXEXCXLIC

SEQ ID NO: 42 - protein, 11 aa, synthetic construct
  MOD_RES 1: note = Glutaric acid bearing 1-amino-1-deoxy-D-glucitol is attached to D through 4-amino-1-carboxymethylpiperidine
  SITE 3: note = X is 5-chloro-L-tryptophan
  SITE 4: note = X is 4-(R)-hydroxy-L-proline
  SITE 6: note = X is D-leucine
  SITE 8: note = X is 4-aminopiperidine-4-carboxylic acid DOTA conjugate
  MOD_RES 11: note = D-lysine conjugated to monodisperse polyethylene glycol 36 units conjugated with acetyl
  CROSSLNK 7..11: note = cysteine cysteine connected through 2,6-lutidylidene
DWXXEXCXLIC

SEQ ID NO: 43 - protein, 11 aa, synthetic construct
  MOD_RES 1: note = Monodisperse polyethylene glycol 36 units conjugated with acetyl attached to 4-amino-1-carboxymethylpiperidine, whereby 4-amino-1-carboxymethylpiperidine is attached to D
  SITE 3: note = X is 5-chloro-L-tryptophan
  SITE 4: note = X is 4-(R)-hydroxy-L-proline
  SITE 6: note = X is D-leucine
  SITE 8: note = X is 4-aminopiperidine-4-carboxylic acid DOTA conjugate
  MOD_RES 11: note = 1-amino-1-deoxy-D-glucitol attached to D-glutamic acid
  CROSSLNK 7..11: note = cysteine cysteine connected through 2,6-lutidylidene
DWXXEXCXLIC

SEQ ID NO: 44 - protein, 11 aa, synthetic construct
  MOD_RES 1: note = Monodisperse polyethylene glycol 36 units conjugated with acetyl attached to 4-amino-1-carboxymethylpiperidine, whereby 4-amino-1-carboxymethylpiperidine is attached to D
  SITE 3: note = X is 5-chloro-L-tryptophan
  SITE 4: note = X is 4-(R)-hydroxy-L-proline
  SITE 6: note = X is D-leucine
  SITE 8: note = X is 4-aminopiperidine-4-carboxylic acid DOTA conjugate
  MOD_RES 11: note = D-glutamic acid attached through 4-amino-1-carboxymethylpiperidine to C-terminus with 1-amino-1-deoxy-D-glucitol being attached to D-glutamic acid
  CROSSLNK 7..11: note = cysteine cysteine connected through 2,6-lutidylidene
DWXXEXCXLIC

SEQ ID NO: 45 - protein, 11 aa, synthetic construct
  MOD_RES 1: note = Monodisperse polyethylene glycol 36 units conjugated with acetyl attached to 4-amino-1-carboxymethylpiperidine, whereby 4-amino-1-carboxymethylpiperidine is attached to D
  SITE 3: note = X is 5-chloro-L-tryptophan
  SITE 4: note = X is (2S,4R)-4-amino-1-pyrrolidin-2-carboxylic acid
  SITE 6: note = X is D-leucine
  SITE 8: note = X is 4-aminopiperidine-4-carboxylic acid with DOTA attached
  MOD_RES 11: note = D-glutamic acid attached through 4-amino-1-carboxymethylpiperidine to C-terminus with 1-amino-1-deoxy-D-glucitol being attached to D-glutamic acid
  CROSSLNK 7..11: note = cysteine cysteine connected through 2,6-lutidylidene
DWXXEXCXLIC

SEQ ID NO: 46 - protein, 11 aa, synthetic construct
  MOD_RES 1: note = Glutaric acid bearing 1-amino-1-deoxy-D-glucitol is attached to D through 4-amino-1-carboxymethylpiperidine
  SITE 3: note = X is 5-chloro-L-tryptophan
  SITE 4: note = X is 4-(R)-hydroxy-L-proline
  SITE 6: note = X is D-leucine
  SITE 8: note = X is 4-aminopiperidine-4-carboxylic acid (glu(DOTA/AGLU)) conjugate
  MOD_RES 11: note = D-glutamic acid attached through 4-amino-1-carboxymethylpiperidine to C-terminus with 1-amino-1-deoxy-D-glucitol being attached to D-glutamic acid
  CROSSLNK 7..11: note = cysteine cysteine connected through 2,6-lutidylidene
DWXXEXCXLIC

SEQ ID NO: 47 - protein, 11 aa, synthetic construct
  MOD_RES 1: note = DOTA attached through 8-amino-3,6-dioxaoctanoic acid to D-glutamic acid bearing 1-amino-1-deoxy-D-glucitol, whereby the D-glutamic acid is attached to D
  SITE 3: note = X is 5-chloro-L-tryptophan
  SITE 4: note = X is (2S,4R)-4-amino-1-pyrrolidin-2-carboxylic acid
  SITE 6: note = X is D-leucine
  SITE 8: note = X is 4-aminopiperidine-4-carboxylic acid to which 1-amino-1-deoxy-D-glucitol is attached through glutaric acid
  MOD_RES 11: note = D-glutamic acid attached through 4-amino-1-carboxymethylpiperidine to C-terminus with 1-amino-1-deoxy-D-glucitol being attached to D-glutamic acid
  CROSSLNK 7..11: note = cysteine cysteine connected through 2,6-lutidylidene
DWXXEXCXLIC

SEQ ID NO: 48 - protein, 11 aa, synthetic construct
  MOD_RES 1: note = DOTA attached to D-glutamic acid bearing 1-amino-1-deoxy-D-glucitol, whereby the D-glutamic acid is attached D
  SITE 3: note = X is 5-chloro-L-tryptophan
  SITE 4: note = X is (2S,4R)-4-amino-1-pyrrolidin-2-carboxylic acid
  SITE 6: note = X is D-leucine
  SITE 8: note = X is 4-aminopiperidine-4-carboxylic acid to which 1-amino-1-deoxy-D-glucitol is attached through glutaric acid
  MOD_RES 11: note = D-glutamic acid attached through 4-amino-1-carboxymethylpiperidine to C-terminus with 1-amino-1-deoxy-D-glucitol being attached to D-glutamic acid
  CROSSLNK 7..11: note = cysteine cysteine connected through 2,6-lutidylidene
DWXXEXCXLIC

SEQ ID NO: 49 - protein, 11 aa, synthetic construct
  MOD_RES 1: note = Glutaric acid bearing 1-amino-1-deoxy-D-glucitol is attached to S through 4-amino-1-carboxymethylpiperidine
  SITE 3: note = X is 5-chloro-L-tryptophan
  SITE 4: note = X is (2S,4R)-4-amino-1-pyrrolidin-2-carboxylic acid
  SITE 5: note = X is L-homoserine
  SITE 6: note = X is D-leucine
  SITE 8: note = X is 4-aminopiperidine-4-carboxylic acid DOTA conjugate
  MOD_RES 11: note = D-glutamic acid attached through 4-amino-1-carboxymethylpiperidine to C-terminus with 1-amino-1-deoxy-D-glucitol being attached to D-glutamic acid
  CROSSLNK 7..11: note = cysteine cysteine connected through 2,6-lutidylidene
SWXXXXCXLIC

SEQ ID NO: 50 - protein, 11 aa, synthetic construct
  MOD_RES 1: note = Glutaric acid bearing 1-amino-1-deoxy-D-glucitol is attached to L-homoserine through 4-amino-1-carboxymethylpiperidine
  SITE 1: note = X is L-homoserine
  SITE 3: note = X is 5-chloro-L-tryptophan
  SITE 4: note = X is 4-(R)-hydroxy-L-proline
  SITE 6: note = X is D-leucine
  SITE 8: note = X is 4-aminopiperidine-4-carboxylic acid (glu(DOTA/AGLU)) conjugate
  MOD_RES 11: note = D-glutamic acid attached through 4-amino-1-carboxymethylpiperidine to C-terminus with 1-amino-1-deoxy-D-glucitol being attached to D-glutamic acid
  CROSSLNK 7..11: note = cysteine cysteine connected through 2,6-lutidylidene
XWXXEXCXLIC

SEQ ID NO: 51 - protein, 11 aa, synthetic construct
  MOD_RES 1: note = Glutaric acid bearing 1-amino-1-deoxy-D-glucitol is attached to S through 4-amino-1-carboxymethylpiperidine
  SITE 3: note = X is 5-chloro-L-tryptophan
  SITE 4: note = X is (2S,4R)-4-amino-1-pyrrolidin-2-carboxylic acid
  SITE 5: note = X is L-homoserine
  SITE 6: note = X is D-leucine
  SITE 8: note = X is 4-aminopiperidine-4-carboxylic acid with DOTA attached
  MOD_RES 11: note = Glutaric acid bearing 1-amino-1-deoxy-D-glucitol is attached to S through 4-amino-1-carboxymethylpiperidine
  CROSSLNK 7..11: note = cysteine cysteine connected through 2,6-lutidylidene
SWXXXXCXLIC

SEQ ID NO: 52 - protein, 11 aa, synthetic construct
  MOD_RES 1: note = Glutaric acid bearing 1-amino-1-deoxy-D-glucitol is attached to L-homoserine through 4-amino-1-carboxymethylpiperidine
  SITE 1: note = X is L-homoserine
  SITE 3: note = X is 5-chloro-L-tryptophan
  SITE 4: note = X is 4-(R)-hydroxy-L-proline
  SITE 6: note = X is D-leucine
  SITE 8: note = X is 4-aminopiperidine-4-carboxylic acid DOTA conjugate
  MOD_RES 11: note = D-glutamic acid attached through 4-amino-1-carboxymethylpiperidine to C-terminus with 1-amino-1-deoxy-D-glucitol being attached to D-glutamic acid
  CROSSLNK 7..11: note = cysteine cysteine connected through 2,6-lutidylidene
XWXXEXCXLIC

SEQ ID NO: 53 - protein, 11 aa, synthetic construct
  MOD_RES 1: note = Glutaric acid bearing 1-amino-1-deoxy-D-glucitol is attached to L-homoserine through 4-amino-1-carboxymethylpiperidine
  SITE 1: note = X is L-homoserine
  SITE 3: note = X is 5-chloro-L-tryptophan
  SITE 4: note = X is (2S,4R)-4-amino-1-pyrrolidin-2-carboxylic acid
  SITE 6: note = X is D-leucine
  SITE 8: note = X is 4-aminopiperidine-4-carboxylic acid with DOTA attached
  MOD_RES 11: note = D-glutamic acid attached through 4-amino-1-carboxymethylpiperidine to C-terminus with 1-amino-1-deoxy-D-glucitol being attached to D-glutamic acid
  CROSSLNK 7..11: note = cysteine cysteine connected through 2,6-lutidylidene
XWXXEXCXLIC

SEQ ID NO: 54 - protein, 11 aa, synthetic construct
  MOD_RES 1: note = 1-amino-1-deoxy-D-glucitol attached through glutaric acid to D
  SITE 3: note = X is 5-chloro-L-tryptophan
  SITE 4: note = X is (2S,4R)-4-amino-1-pyrrolidin-2-carboxylic acid
  SITE 6: note = X is D-leucine
  SITE 7: note = X is D-cysteine
  SITE 8: note = X is 4-aminopiperidine-4-carboxylic acid DOTA conjugate
  MOD_RES 11: note = D-glutamic acid attached through 4-amino-1-carboxymethylpiperidine to C-terminus with 1-amino-1-deoxy-D-glucitol being attached to D-glutamic acid
  CROSSLNK 7..11: note = cysteine cysteine connected through 2,6-lutidylidene
DWXXEXXXLIC

SEQ ID NO: 55 - protein, 11 aa, synthetic construct
  MOD_RES 1: note = 1-amino-1-deoxy-D-glucitol attached through glutaric acid to D
  SITE 3: note = X is 5-chloro-L-tryptophan
  SITE 4: note = X is (2S,4R)-4-amino-1-pyrrolidin-2-carboxylic acid
  SITE 6: note = X is D-leucine
  SITE 7: note = X is D-cysteine
  SITE 8: note = X is 4-aminopiperidine-4-carboxylic acid with DOTA attached
  SITE 11: note = X is D-cysteine
  CROSSLNK 7..11: note = cysteine cysteine connected through 2,6-lutidylidene
DWXXEXXXLIX

SEQ ID NO: 56 - protein, 11 aa, synthetic construct
  MOD_RES 1: note = DOTA attached to D through 1,13-diamino-4,7,10-trioxatridecan-succinamic acid
  SITE 3: note = X is 5-chloro-L-tryptophan
  SITE 6: note = X is D-leucine
  SITE 8: note = X is 4-aminopiperidine-4-carboxylic acid DOTA conjugate
  SITE 11: note = X is 2-aminoethane-1-thiol
  CROSSLNK 7..11: note = cysteine 2-aminoethane-1-thiol through 3-methylbenzylidene
DWXPEXCXLIX

SEQ ID NO: 57 - protein, 12 aa, synthetic construct
  MOD_RES 1: note = DOTA attached to D through 1,13-diamino-4,7,10-trioxatridecan-succinamic acid
  SITE 3: note = X is 5-chloro-L-tryptophan
  SITE 4: note = X is 4-(R)-hydroxy-L-proline
  SITE 6: note = X is D-leucine
  SITE 8: note = X is 4-amino-tetrahydropyran-4-carboxylic acid
  MOD_RES 12: note = DOTA attached to K
  CROSSLNK 7..11: note = cysteine cysteine connected through 2,6-lutidylidene
DWXXEXCXLICK

SEQ ID NO: 58 - protein, 12 aa, synthetic construct
  MOD_RES 1: note = DOTA attached to D through 1,13-diamino-4,7,10-trioxatridecan-succinamic acid
  SITE 3: note = X is 5-chloro-L-tryptophan
  SITE 4: note = X is 4-(R)-hydroxy-L-proline
  SITE 6: note = X is D-leucine
  SITE 8: note = X is 4-aminopiperidine-4-carboxylic acid DOTA conjugate
  MOD_RES 12: note = DOTA attached to K
  CROSSLNK 7..11: note = cysteine cysteine connected through 2,6-lutidylidene
DWXXEXCXLICK

SEQ ID NO: 59 - protein, 12 aa, synthetic construct
  MOD_RES 1: note = DOTA attached to D through 1,13-diamino-4,7,10-trioxatridecan-succinamic acid
  SITE 3: note = X is 5-chloro-L-tryptophan
  SITE 4: note = X is (2S,4R)-4-amino-1-pyrrolidin-2-carboxylic acid with DOTA attached
  SITE 6: note = X is D-leucine
  SITE 8: note = X is 4-aminopiperidine-4-carboxylic acid with DOTA attached
  MOD_RES 12: note = DOTA attached to K
  CROSSLNK 7..11: note = cysteine cysteine connected through 2,6-lutidylidene
DWXXEXCXLICK

SEQ ID NO: 60 - protein, 11 aa, synthetic construct
  MOD_RES 1: note = Glutaric acid bearing 1-amino-1-deoxy-D-glucitol is attached to D through 4-amino-1-carboxymethylpiperidine
  SITE 3: note = X is 5-chloro-L-tryptophan
  SITE 4: note = X is (2S,4R)-4-amino-1-pyrrolidin-2-carboxylic acid
  SITE 6: note = X is D-leucine
  SITE 8: note = X is Apc(GaDOTA-glu(AGLU)) = 4-aminopiperidine-4-carboxylic acid (glu(GaDOTA/AGLU)) conjugate
  MOD_RES 11: note = D-glutamic acid attached through 4-amino-1-carboxymethylpiperidine to C-terminus with 1-amino-1-deoxy-D-glucitol being attached to D-glutamic acid
  CROSSLNK 7..11: note = cysteine cysteine connected through 2,6-lutidylidene
DWXXEXCXLIC

SEQ ID NO: 61 - protein, 11 aa, synthetic construct
  MOD_RES 1: note = Glutaric acid bearing 1-amino-1-deoxy-D-glucitol is attached to D through 4-amino-1-carboxymethylpiperidine
  SITE 3: note = X is 5-chloro-L-tryptophan
  SITE 4: note = X is (2S,4R)-4-amino-1-pyrrolidin-2-carboxylic acid
  SITE 6: note = X is D-leucine
  SITE 8: note = X is 4-aminopiperidine-4-carboxylic acid (glu(InDOTA/AGLU)) conjugate
  MOD_RES 11: note = D-glutamic acid attached through 4-amino-1-carboxymethylpiperidine to C-terminus with 1-amino-1-deoxy-D-glucitol being attached to D-glutamic acid
  CROSSLNK 7..11: note = cysteine cysteine connected through 2,6-lutidylidene
DWXXEXCXLIC

SEQ ID NO: 62 - protein, 11 aa, synthetic construct
  MOD_RES 1: note = Glutaric acid bearing 1-amino-1-deoxy-D-glucitol is attached to D through 4-amino-1-carboxymethylpiperidine
  SITE 3: note = X is 5-chloro-L-tryptophan
  SITE 4: note = X is (2S,4R)-4-amino-1-pyrrolidin-2-carboxylic acid
  SITE 6: note = X is D-leucine
  SITE 8: note = 4-aminopiperidine-4-carboxylic acid (glu(LuDOTA/AGLU)) conjugate
  MOD_RES 11: note = D-glutamic acid attached through 4-amino-1-carboxymethylpiperidine to C-terminus with 1-amino-1-deoxy-D-glucitol being attached to D-glutamic acid
  CROSSLNK 7..11: note = cysteine cysteine connected through 2,6-lutidylidene
DWXXEXCXLIC

SEQ ID NO: 63 - protein, 11 aa, synthetic construct
  MOD_RES 1: note = LuDOTA attached to D-glutamic acid bearing 1-amino-1-deoxy-D-glucitol, whereby the D-glutamic acid is attached through 4-amino-1-carboxymethylpiperidine to N-terminus of D
  SITE 3: note = X is 5-chloro-L-tryptophan
  SITE 4: note = X is 4-(R)-hydroxy-L-proline
  SITE 6: note = X is D-leucine
  SITE 8: note = X is 4-amino-tetrahydropyran-4-carboxylic acid
  MOD_RES 11: note = D-glutamic acid attached through 4-amino-1-carboxymethylpiperidine to C-terminus with 1-amino-1-deoxy-D-glucitol being attached to D-glutamic acid
  CROSSLNK 7..11: note = cysteine cysteine connected through 2,6-lutidylidene
DWXXEXCXLIC

SEQ ID NO: 64 - protein, 11 aa, synthetic construct
  MOD_RES 1: note = InDOTA attached to D-glutamic acid bearing 1-amino-1-deoxy-D-glucitol, whereby the D-glutamic acid is attached through 4-amino-1-carboxymethylpiperidine to N-terminus of D
  SITE 3: note = X is 5-chloro-L-tryptophan
  SITE 4: note = X is 4-(R)-hydroxy-L-proline
  SITE 6: note = X is D-leucine
  SITE 8: note = X is 4-amino-tetrahydropyran-4-carboxylic acid
  MOD_RES 11: note = D-glutamic acid attached through 4-amino-1-carboxymethylpiperidine to C-terminus with 1-amino-1-deoxy-D-glucitol being attached to D-glutamic acid
  CROSSLNK 7..11: note = cysteine cysteine connected through 2,6-lutidylidene
DWXXEXCXLIC

SEQ ID NO: 65 - protein, 11 aa, synthetic construct
  MOD_RES 1: note = EuDOTA attached to D-glutamic acid bearing 1-amino-1-deoxy-D-glucitol, whereby the D-glutamic acid is attached through 4-amino-1-carboxymethylpiperidine to N-terminus of D
  SITE 3: note = X is 5-chloro-L-tryptophan
  SITE 4: note = X is 4-(R)-hydroxy-L-proline
  SITE 6: note = X is D-leucine
  SITE 8: note = X is 4-amino-tetrahydropyran-4-carboxylic acid
  MOD_RES 11: note = D-glutamic acid attached through 4-amino-1-carboxymethylpiperidine to C-terminus with 1-amino-1-deoxy-D-glucitol being attached to D-glutamic acid
  CROSSLNK 7..11: note = cysteine cysteine connected through 2,6-lutidylidene
DWXXEXCXLIC

SEQ ID NO: 66 - protein, 11 aa, synthetic construct
  MOD_RES 1: note = 1-amino-1-deoxy-D-glucitol attached through glutaric acid to D
  SITE 3: note = X is 5-chloro-L-tryptophan
  SITE 4: note = X is 4-(R)-hydroxy-L-proline
  SITE 6: note = X is D-leucine
  SITE 8: note = X is 4-aminopiperidine-4-carboxylic acid InDOTA conjugate
  MOD_RES 11: note = D-glutamic acid attached through 4-amino-1-carboxymethylpiperidine to C-terminus with 1-amino-1-deoxy-D-glucitol being attached to D-glutamic acid
  CROSSLNK 7..11: note = cysteine cysteine connected through 2,6-lutidylidene
DWXXEXCXLIC

SEQ ID NO: 67 - protein, 11 aa, synthetic construct
  MOD_RES 1: note = 1-amino-1-deoxy-D-glucitol attached through glutaric acid to D
  SITE 3: note = X is 5-chloro-L-tryptophan
  SITE 4: note = X is 4-(R)-hydroxy-L-proline
  SITE 6: note = X is D-leucine
  SITE 8: note = X is 4-aminopiperidine-4-carboxylic acid LuDOTA conjugate
  MOD_RES 11: note = D-glutamic acid attached through 4-amino-1-carboxymethylpiperidine to C-terminus with 1-amino-1-deoxy-D-glucitol being attached to D-glutamic acid
  CROSSLNK 7..11: note = cysteine cysteine connected through 2,6-lutidylidene
DWXXEXCXLIC

SEQ ID NO: 68 - protein, 11 aa, synthetic construct
  MOD_RES 1: note = 1-amino-1-deoxy-D-glucitol attached through glutaric acid to D
  SITE 3: note = X is 5-chloro-L-tryptophan
  SITE 4: note = X is (2S,4R)-4-amino-1-pyrrolidin-2-carboxylic acid
  SITE 6: note = X is D-leucine
  SITE 8: note = X is 4-aminopiperidine-4-carboxylic acid LuDOTA conjugate
  MOD_RES 11: note = D-glutamic acid attached through 4-amino-1-carboxymethylpiperidine to C-terminus with 1-amino-1-deoxy-D-glucitol being attached to D-glutamic acid
  CROSSLNK 7..11: note = cysteine cysteine connected through 2,6-lutidylidene
DWXXEXCXLIC

SEQ ID NO: 69 - protein, 11 aa, synthetic construct
  MOD_RES 1: note = 1-amino-1-deoxy-D-glucitol attached through glutaric acid to D
  SITE 3: note = X is 5-chloro-L-tryptophan
  SITE 4: note = X is (2S,4R)-4-amino-1-pyrrolidin-2-carboxylic acid
  SITE 6: note = X is D-leucine
  SITE 8: note = X is 4-aminopiperidine-4-carboxylic acid InDOTA conjugate
  MOD_RES 11: note = D-glutamic acid attached through 4-amino-1-carboxymethylpiperidine to C-terminus with 1-amino-1-deoxy-D-glucitol being attached to D-glutamic acid
  CROSSLNK 7..11: note = cysteine cysteine connected through 2,6-lutidylidene
DWXXEXCXLIC

SEQ ID NO: 70 - protein, 11 aa, synthetic construct
  MOD_RES 1: note = 1-amino-1-deoxy-D-glucitol attached through glutaric acid to D
  SITE 3: note = X is 5-chloro-L-tryptophan
  SITE 4: note = X is (2S,4R)-4-amino-1-pyrrolidin-2-carboxylic acid
  SITE 6: note = X is D-leucine
  SITE 8: note = X is 4-aminopiperidine-4-carboxylic acid with GaDOTA attached
  MOD_RES 11: note = D-glutamic acid attached through 4-amino-1-carboxymethylpiperidine to C-terminus with 1-amino-1-deoxy-D-glucitol being attached to D-glutamic acid
  CROSSLNK 7..11: note = cysteine cysteine connected through 2,6-lutidylidene
DWXXEXCXLIC

SEQ ID NO: 71 - protein, 11 aa, synthetic construct
  MOD_RES 1: note = 1-amino-1-deoxy-D-glucitol attached through glutaric acid to D
  SITE 3: note = X is 5-chloro-L-tryptophan
  SITE 4: note = X is 4-(R)-hydroxy-L-proline
  SITE 6: note = X is D-leucine
  SITE 8: note = X is 4-aminopiperidine-4-carboxylic acid with GaDOTA attached
  MOD_RES 11: note = D-glutamic acid attached through 4-amino-1-carboxymethylpiperidine to C-terminus with 1-amino-1-deoxy-D-glucitol being attached to D-glutamic acid
  CROSSLNK 7..11: note = cysteine cysteine connected through 2,6-lutidylidene
DWXXEXCXLIC

SEQ ID NO: 72 - protein, 11 aa, synthetic construct
  MOD_RES 1: note = 1-amino-1-deoxy-D-glucitol attached through glutaric acid to D
  SITE 3: note = X is 5-chloro-L-tryptophan
  SITE 4: note = X is (2S,4R)-4-amino-1-pyrrolidin-2-carboxylic acid
  SITE 6: note = X is D-leucine
  SITE 8: note = X is 4-aminopiperidine-4-carboxylic acid with GaNODAGA attached
  MOD_RES 11: note = D-glutamic acid attached through 4-amino-1-carboxymethylpiperidine to C-terminus with 1-amino-1-deoxy-D-glucitol being attached to D-glutamic acid
  CROSSLNK 7..11: note = cysteine cysteine connected through 2,6-lutidylidene
DWXXEXCXLIC

SEQ ID NO: 73 - protein, 11 aa, synthetic construct
  MOD_RES 1: note = 1-amino-1-deoxy-D-glucitol attached through glutaric acid to D
  SITE 3: note = X is 5-chloro-L-tryptophan
  SITE 4: note = X is 4-(R)-hydroxy-L-proline
  SITE 6: note = X is D-leucine
  SITE 8: note = X is 4-aminopiperidine-4-carboxylic acid DOTA conjugate
  SITE 11: note = X is 2-aminoethane-1-thiol
  CROSSLNK 7..11: note = cysteine 2-aminoethane-1-thiol through 2,6-lutidylidene
DWXXEXCXLIX

SEQ ID NO: 74 - protein, 10 aa, synthetic construct
  MOD_RES 1: note = HO-succinyl attached to W
  SITE 2: note = X is 5-chloro-L-tryptophan
  SITE 3: note = X is 4-(R)-hydroxy-L-proline
  SITE 5: note = X is D-leucine
  MOD_RES 10: note = DOTA bearing K attached to C through 4-amino-1-carboxymethylpiperidine
  CROSSLNK 7..10: note = cysteine cysteine connected through 2,6-lutidylidene
WXXEXCXLIC

SEQ ID NO: 75 - protein, 11 aa, synthetic construct
  MOD_RES 1: note = Acetyl attached to D
  SITE 3: note = X is 3-(1-naphthyl)-L-alanine
  SITE 8: note = X is 1-amino-isobutyric acid
  MOD_RES 11: note = DOTA bearing L-lysine attached to C through 1,13-diamino-4,7,10-trioxatridecan-succinamic acid
  CROSSLNK 7..11: note = cysteine cysteine connected through 3-methylbenzylidene
DWXPELCXLIC

SEQ ID NO: 76 - protein, 11 aa, synthetic construct
  MOD_RES 1: note = Acetyl attached to D
  SITE 3: note = X is 3-(3-benzothienyl)-L-alanine
  SITE 8: note = X is 1-amino-isobutyric acid
  MOD_RES 11: note = DOTA bearing L-lysine attached to C through 1,13-diamino-4,7,10-trioxatridecan-succinamic acid
  CROSSLNK 7..11: note = cysteine cysteine connected through 3-methylbenzylidene
DWXPELCXLIC

SEQ ID NO: 77 - protein, 11 aa, synthetic construct
  MOD_RES 1: note = Acetyl attached to D
  SITE 3: note = X is 5-chloro-L-tryptophan
  SITE 8: note = X is 1-amino-isobutyric acid
  MOD_RES 11: note = bearing L-lysine attached to C through 1,13-diamino-4,7,10-trioxatridecan-succinamic acid
  CROSSLNK 7..11: note = cysteine cysteine connected through 3-methylbenzylidene
DWXPELCXLIC

SEQ ID NO: 78 - protein, 11 aa, synthetic construct
  MOD_RES 1: note = DOTA attached to D through 1,13-diamino-4,7,10-trioxatridecan-succinamic acid
  SITE 3: note = X is 5-chloro-L-tryptophan
  SITE 6: note = X is D-leucine
  SITE 8: note = X is 1-amino-isobutyric acid
  SITE 11: note = X is 2-aminoethane-1-thiol
  CROSSLNK 7..11: note = cysteine 2-aminoethane-1-thiol through 3-methylbenzylidene
DWXPEXCXLIX

SEQ ID NO: 79 - protein, 11 aa, synthetic construct
  MOD_RES 1: note = Acetyl attached to D
  SITE 3: note = X is 3-(1-naphthyl)-L-alanine
  SITE 6: note = X is D-leucine
  SITE 8: note = X is 1-amino-isobutyric acid
  MOD_RES 11: note = DOTA bearing L-lysine attached to C through 1,13-diamino-4,7,10-trioxatridecan-succinamic acid
  CROSSLNK 7..11: note = cysteine cysteine connected through 3-methylbenzylidene
DWXPEXCXLIC

SEQ ID NO: 80 - protein, 11 aa, synthetic construct
  MOD_RES 1: note = Acetyl attached to D
  SITE 3: note = X is 5-chloro-L-tryptophan
  SITE 6: note = X is D-leucine
  SITE 8: note = X is 1-amino-isobutyric acid
  MOD_RES 11: note = DOTA bearing L-lysine attached to C through 1,13-diamino-4,7,10-trioxatridecan-succinamic acid
  CROSSLNK 7..11: note = cysteine cysteine connected through 3-methylbenzylidene
DWXPEXCXLIC

SEQ ID NO: 81 - protein, 11 aa, synthetic construct
  MOD_RES 1: note = Acetyl attached to D
  SITE 3: note = X is 3-(1-naphthyl)-L-alanine
  SITE 6: note = X is D-leucine
  SITE 8: note = X is 1-Amino-1-cyclohexan-carboxylic acid
  MOD_RES 11: note = DOTA bearing L-lysine attached to C through 1,13-diamino-4,7,10-trioxatridecan-succinamic acid
  CROSSLNK 7..11: note = cysteine cysteine connected through 3-methylbenzylidene
DWXPEXCXLIC

SEQ ID NO: 82 - protein, 11 aa, synthetic construct
  MOD_RES 1: note = Acetyl attached to D
  SITE 3: note = X is 5-fluoro-L-tryptophan
  SITE 6: note = X is D-leucine
  SITE 8: note = X is 1-amino-isobutyric acid
  MOD_RES 11: note = DOTA bearing L-lysine attached to C through 1,13-diamino-4,7,10-trioxatridecan-succinamic acid
  CROSSLNK 7..11: note = cysteine cysteine connected through 3-methylbenzylidene
DWXPEXCXLIC

SEQ ID NO: 83 - protein, 11 aa, synthetic construct
  MOD_RES 1: note = Acetyl attached to D
  SITE 3: note = X is 5-bromo-L-tryptophan
  SITE 6: note = X is D-leucine
  SITE 8: note = X is 1-amino-isobutyric acid
  MOD_RES 11: note = DOTA bearing L-lysine attached to C through 1,13-diamino-4,7,10-trioxatridecan-succinamic acid
  CROSSLNK 7..11: note = cysteine cysteine connected through 3-methylbenzylidene
DWXPEXCXLIC

SEQ ID NO: 84 - protein, 11 aa, synthetic construct
  MOD_RES 1: note = Acetyl attached to D
  SITE 2: note = X is 5-fluoro-L-tryptophan
  SITE 3: note = X is 3-(1-naphthyl)-L-alanine
  SITE 6: note = X is D-leucine
  SITE 8: note = X is 1-amino-isobutyric acid
  MOD_RES 11: note = DOTA bearing L-lysine attached to C through 1,13-diamino-4,7,10-trioxatridecan-succinamic acid
  CROSSLNK 7..11: note = cysteine cysteine connected through 3-methylbenzylidene
DXXPEXCXLIC

SEQ ID NO: 85 - protein, 11 aa, synthetic construct
  MOD_RES 1: note = Acetyl attached to D
  SITE 2: note = X is 6-fluoro-L-tryptophan
  SITE 3: note = X is 3-(1-naphthyl)-L-alanine
  SITE 6: note = X is D-leucine
  SITE 8: note = X is 1-amino-isobutyric acid
  MOD_RES 11: note = DOTA bearing L-lysine attached to C through 1,13-diamino-4,7,10-trioxatridecan-succinamic acid
  CROSSLNK 7..11: note = cysteine cysteine connected through 3-methylbenzylidene
DXXPEXCXLIC

SEQ ID NO: 86 - protein, 10 aa, synthetic construct
  MOD_RES 1: note = HO-succinyl attached to W
  SITE 2: note = X is 3-(1-naphthyl)-L-alanine
  SITE 8: note = X is 1-amino-isobutyric acid
  MOD_RES 10: note = DOTA bearing L-lysine attached to C through 1,13-diamino-4,7,10-trioxatridecan-succinamic acid
  CROSSLNK 6..10: note = cysteine cysteine connected through 3-methylbenzylidene
WXPELCXLIC

SEQ ID NO: 87 - protein, 11 aa, synthetic construct
  MOD_RES 1: note = DOTA attached to D through 1,13-diamino-4,7,10-trioxatridecan-succinamic acid
  SITE 3: note = X is 5-chloro-L-tryptophan
  SITE 6: note = X is D-leucine
  SITE 8: note = X is 1-Amino-1-cyclohexan-carboxylic acid
  SITE 11: note = X is 2-aminoethane-1-thiol
  CROSSLNK 7..11: note = cysteine 2-aminoethane-1-thiol through 3-methylbenzylidene
DWXPEXCXLIX

SEQ ID NO: 88 - protein, 11 aa, synthetic construct
  MOD_RES 1: note = DOTA attached to D through 1,13-diamino-4,7,10-trioxatridecan-succinamic acid
  SITE 3: note = X is 5-chloro-L-tryptophan
  SITE 6: note = X is D-leucine
  SITE 8: note = X is 4-amino-tetrahydropyran-4-carboxylic acid
  SITE 11: note = X is 2-aminoethane-1-thiol
  CROSSLNK 7..11: note = cysteine 2-aminoethane-1-thiol through 3-methylbenzylidene
DWXPEXCXLIX

SEQ ID NO: 89 - protein, 11 aa, synthetic construct
  MOD_RES 1: note = DOTA attached to D through 1,13-diamino-4,7,10-trioxatridecan-succinamic acid
  SITE 3: note = X is 5-chloro-L-tryptophan
  SITE 6: note = X is D-leucine
  SITE 8: note = X is 4-aminopiperidine-4-carboxylic acid
  SITE 11: note = X is 2-aminoethane-1-thiol
  CROSSLNK 7..11: note = cysteine 2-aminoethane-1-thiol through 3-methylbenzylidene
DWXPEXCXLIX

SEQ ID NO: 90 - protein, 11 aa, synthetic construct
  MOD_RES 1: note = DOTA attached to D through 4-amino-1-carboxymethylpiperidine
  SITE 3: note = X is 5-chloro-L-tryptophan
  SITE 6: note = X is D-leucine
  SITE 8: note = X is 1-amino-isobutyric acid
  SITE 11: note = X is 2-aminoethane-1-thiol
  CROSSLNK 7..11: note = cysteine 2-aminoethane-1-thiol through 3-methylbenzylidene
DWXPEXCXLIX

SEQ ID NO: 91 - protein, 11 aa, synthetic construct
  MOD_RES 1: note = DOTA attached to D through 1,13-diamino-4,7,10-trioxatridecan-succinamic acid
  SITE 3: note = X is 5-chloro-L-tryptophan
  SITE 6: note = X is D-leucine
  SITE 8: note = X is 1-amino-isobutyric acid
  SITE 11: note = X is 2-aminoethane-1-thiol
  CROSSLNK 7..11: note = cysteine 2-aminoethane-1-thiol through 2,6-lutidylidene
DWXPEXCXLIX

SEQ ID NO: 92 - protein, 11 aa, synthetic construct
  MOD_RES 1: note = DOTA attached to D through 1,13-diamino-4,7,10-trioxatridecan-succinamic acid
  SITE 3: note = X is 5-chloro-L-tryptophan
  SITE 6: note = X is D-leucine
  SITE 11: note = X is 2-aminoethane-1-thiol
  CROSSLNK 7..11: note = cysteine 2-aminoethane-1-thiol connected through 3,5-lutidylidene
DWXPEXCXLIX

SEQ ID NO: 93 - protein, 11 aa, synthetic construct
  MOD_RES 1: note = DOTA attached to D through 1,13-diamino-4,7,10-trioxatridecan-succinamic acid
  SITE 3: note = X is 5-chloro-L-tryptophan
  SITE 6: note = X is D-leucine
  SITE 7: note = X is (S)-2,3-Diaminopropionic acid
  SITE 8: note = X is 1-amino-isobutyric acid
  SITE 11: note = X is 2-aminoethane-1-thiol
  CROSSLNK 7..11: note = (S)-2,3-Diaminopropionic acid 2-aminoethane-1-thiol connected through3-methylbenzylidene
DWXPEXXXLIX

SEQ ID NO: 94 - protein, 10 aa, synthetic construct
  MOD_RES 1: note = DOTA attached to D through 1,13-diamino-4,7,10-trioxatridecan-succinamic acid
  SITE 3: note = X is 5-chloro-L-tryptophan
  SITE 6: note = X is D-leucine
  SITE 8: note = X is 1-amino-isobutyric acid
  MOD_RES 10: note = ethylendiamine attached to I
  CROSSLNK 7..10: note = cysteine isoleucine conjugated to ethylendiamine
DWXPEXCXLI

SEQ ID NO: 95 - protein, 11 aa, synthetic construct
  MOD_RES 1: note = DOTA attached to D through 1,13-diamino-4,7,10-trioxatridecan-succinamic acid
  SITE 3: note = X is 5-chloro-L-tryptophan
  SITE 4: note = X is 4-(R)-hydroxy-L-proline
  SITE 6: note = X is D-leucine
  SITE 8: note = X is 1-amino-isobutyric acid
  MOD_RES 11: note = X is 2-aminoethane-1-thiol
  CROSSLNK 7..11: note = cysteine 2-aminoethane-1-thiol connected through 3-methylbenzylidene
DWXXEXCXLIX

SEQ ID NO: 96 - protein, 11 aa, unidentified
  MOD_RES 1: note = DOTA attached to D through 1,13-diamino-4,7,10-trioxatridecan-succinamic acid
  SITE 3: note = X is 5-chloro-L-tryptophan
  SITE 4: note = X is (2S,4R)-4-amino-1-pyrrolidin-2-carboxylic acid
  SITE 6: note = X is D-leucine
  SITE 8: note = X is 1-amino-isobutyric acid
  SITE 11: note = X is 2-aminoethane-1-thiol
  CROSSLNK 7..11: note = cysteine 2-aminoethane-1-thiol connected through 3-methylbenzylidene
DWXXEXCXLIX

SEQ ID NO: 97 - protein, 11 aa, synthetic construct
  MOD_RES 1: note = DOTA attached to D through 1,13-diamino-4,7,10-trioxatridecan-succinamic acid
  SITE 3: note = X is 5-chloro-L-tryptophan
  SITE 4: note = X is 4-(S)-Amino-L-proline
  SITE 6: note = X is D-leucine
  SITE 8: note = X is 1-amino-isobutyric acid
  SITE 11: note = X is 2-aminoethane-1-thiol
  CROSSLNK 7..11: note = cysteine 2-aminoethane-1-thiol connected through 3-methylbenzylidene
DWXXEXCXLIX

SEQ ID NO: 98 - protein, 11 aa, synthetic construct
  MOD_RES 1: note = DOTA attached to D through 1,13-diamino-4,7,10-trioxatridecan-succinamic acid
  SITE 3: note = X is 5-chloro-L-tryptophan
  SITE 6: note = X is D-leucine
  SITE 8: note = X is 4-aminopiperidine-4-carboxylic acid with acetyl attached
  SITE 11: note = X is 2-aminoethane-1-thiol
  CROSSLNK 7..11: note = cysteine 2-aminoethane-1-thiol connected through 3-methylbenzylidene
DWXPEXCXLIX

SEQ ID NO: 99 - protein, 11 aa, synthetic construct
  MOD_RES 1: note = DOTA attached to D through 1,13-diamino-4,7,10-trioxatridecan-succinamic acid
  SITE 3: note = X is 5-chloro-L-tryptophan
  SITE 6: note = X is D-leucine
  SITE 7: note = X is (S)-2,3-Diaminopropionic acid
  SITE 8: note = X is 1-amino-isobutyric acid
  SITE 11: note = X is 2-aminoethane-1-thiol oxidized
  CROSSLNK 7..11: note = (S)-2,3-Diaminopropionic acid 2-aminoethane-1-thiol oxidized connected through 3-methylbenzylidene
DWXPEXXXLIX

SEQ ID NO: 100 - protein, 11 aa, synthetic construct
  MOD_RES 1: note = DOTA attached to D through 1,13-diamino-4,7,10-trioxatridecan-succinamic acid
  SITE 3: note = X is 5-chloro-L-tryptophan
  SITE 6: note = X is D-leucine
  SITE 8: note = X is 1-amino-isobutyric acid
  SITE 9: note = X is L-homoleucine
  SITE 11: note = X is 2-aminoethane-1-thiol
  CROSSLNK 7..11: note = cysteine 2-aminoethane-1-thiol connected through 3-methylbenzylidene
DWXPEXCXXIX

SEQ ID NO: 101 - protein, 11 aa, synthetic construct
  MOD_RES 1: note = DOTA attached to D through 1,13-diamino-4,7,10-trioxatridecan-succinamic acid
  SITE 3: note = X is 5-chloro-L-tryptophan
  SITE 4: note = X is N-methyl-glycine
  SITE 6: note = X is D-leucine
  SITE 8: note = X is 1-amino-isobutyric acid
  SITE 11: note = X is 2-aminoethane-1-thiol
  CROSSLNK 7..11: note = cysteine 2-aminoethane-1-thiol connected through 3-methylbenzylidene
DWXXEXCXLIX

SEQ ID NO: 102 - protein, 11 aa, synthetic construct
  MOD_RES 1: note = DOTA attached to D through 1,13-diamino-4,7,10-trioxatridecan-succinamic acid
  SITE 3: note = X is 5-chloro-L-tryptophan
  SITE 6: note = X is D-leucine
  SITE 8: note = X is alpha-methyl-L-serine
  SITE 11: note = X is 2-aminoethane-1-thiol
  CROSSLNK 7..11: note = cysteine 2-aminoethane-1-thiol connected through 3-methylbenzylidene
DWXPEXCXLIX

SEQ ID NO: 103 - protein, 11 aa, synthetic construct
  MOD_RES 1: note = DOTA attached to D through 1,13-diamino-4,7,10-trioxatridecan-succinamic acid
  SITE 3: note = X is 5-chloro-L-tryptophan
  SITE 4: note = X is 3-(S)-hydroxy-L-proline
  SITE 6: note = X is D-leucine
  SITE 8: note = X is 1-amino-isobutyric acid
  SITE 11: note = X is 2-aminoethane-1-thiol
  CROSSLNK 7..11: note = cysteine 2-aminoethane-1-thiol connected through 3-methylbenzylidene
DWXXEXCXLIX

SEQ ID NO: 104 - protein, 11 aa, synthetic construct
  MOD_RES 1: note = DOTA attached to D through 4-amino-1-carboxymethylpiperidine
  SITE 3: note = X is 5-chloro-L-tryptophan
  SITE 4: note = X is 4-(R)-hydroxy-L-proline
  SITE 6: note = X is D-leucine
  SITE 8: note = X is 4-amino-tetrahydropyran-4-carboxylic acid
  SITE 11: note = X is 2-aminoethane-1-thiol
  CROSSLNK 7..11: note = cysteine 2-aminoethane-1-thiol through 2,6-lutidylidene
DWXXEXCXLIX

SEQ ID NO: 105 - protein, 11 aa, synthetic construct
  MOD_RES 1: note = DOTA attached to D through 1,13-diamino-4,7,10-trioxatridecan-succinamic acid
  SITE 2: note = X is 6-fluoro-L-tryptophan
  SITE 3: note = X is 5-chloro-L-tryptophan
  SITE 6: note = X is D-leucine
  SITE 8: note = X is 1-amino-isobutyric acid
  SITE 11: note = X is 2-aminoethane-1-thiol
  CROSSLNK 7..11: note = cysteine 2-aminoethane-1-thiol connected through 3-methylbenzylidene
DXXPEXCXLIX

SEQ ID NO: 106 - protein, 11 aa, synthetic construct
  MOD_RES 1: note = DOTA attached to D through 1,13-diamino-4,7,10-trioxatridecan-succinamic acid
  SITE 2: note = X is 6-fluoro-L-tryptophan
  SITE 3: note = X is 5-bromo-L-tryptophan
  SITE 6: note = X is D-leucine
  SITE 8: note = X is 1-amino-isobutyric acid
  SITE 11: note = X is 2-aminoethane-1-thiol
  CROSSLNK 7..11: note = cysteine 2-aminoethane-1-thiol connected through 3-methylbenzylidene
DXXPEXCXLIX

SEQ ID NO: 107 - protein, 11 aa, synthetic construct
  MOD_RES 1: note = DOTA attached to D through 4-amino-1-carboxymethylpiperidine
  SITE 3: note = X is 5-chloro-L-tryptophan
  SITE 4: note = X is 4-(R)-hydroxy-L-proline
  SITE 6: note = X is D-leucine
  SITE 8: note = X is 4-amino-tetrahydropyran-4-carboxylic acid
  SITE 11: note = X is 2-aminoethane-1-thiol
  CROSSLNK 7..11: note = (S)-2,3-Diaminopropionic acid 2-aminoethane-1-thiol connected through 2,6-lutidylidene
DWXXEXXXLIX

SEQ ID NO: 108 - protein, 11 aa, synthetic construct
  MOD_RES 1: note = DOTA attached to D through 4-amino-1-carboxymethylpiperidine
  SITE 3: note = X is 5-chloro-L-tryptophan
  SITE 4: note = X is 4-(R)-hydroxy-L-proline
  SITE 6: note = X is D-leucine
  SITE 8: note = X is 4-amino-tetrahydropyran-4-carboxylic acid
  SITE 11: note = X is 2-aminoethane-1-thiol
  CROSSLNK 7..11: note = (S)-2,3-Diaminopropionic acid 2-aminoethane-1-thiol connected through 3-methylbenzylidene
DWXXEXXXLIX

SEQ ID NO: 109 - protein, 10 aa, synthetic construct
  MOD_RES 1: note = HO-succinyl attached to W
  SITE 2: note = X is 5-chloro-L-tryptophan
  SITE 3: note = X is 4-(R)-hydroxy-L-proline
  SITE 6: note = X is D-leucine
  SITE 8: note = X is 4-amino-tetrahydropyran-4-carboxylic acid
  MOD_RES 10: note = DOTA bearing L-lysine attached to C through 1,13-diamino-4,7,10-trioxatridecan-succinamic acid
  CROSSLNK 6..10: note = cysteine cysteine connected through 2,6-lutidylidene
WXXEXCXLIC

SEQ ID NO: 110 - protein, 10 aa, synthetic construct
  MOD_RES 1: note = HO-succinyl attached to W
  SITE 2: note = X is 5-chloro-L-tryptophan
  SITE 3: note = X is 4-(R)-hydroxy-L-proline
  SITE 5: note = X is D-leucine
  SITE 7: note = X is 4-amino-tetrahydropyran-4-carboxylic acid
  CROSSLNK 6..10: note = cysteine cysteine connected through 2,6-lutidylidene
WXXEXCXLIC

SEQ ID NO: 111 - protein, 10 aa, synthetic construct
  MOD_RES 1: note = HO-succinyl attached to W
  SITE 2: note = X is 5-chloro-L-tryptophan
  SITE 3: note = X is 4-(R)-hydroxy-L-proline
  SITE 5: note = X is D-leucine
  SITE 7: note = X is 4-aminopiperidine-4-carboxylic acid DOTA conjugate
  SITE 10: note = X is 2-aminoethane-1-thiol
  CROSSLNK 6..10: note = cysteine 2-aminoethane-1-thiol through 2,6-lutidylidene
WXXEXCXLIX

SEQ ID NO: 112 - protein, 11 aa, synthetic construct
  MOD_RES 1: note = DOTA attached to D through 1,13-diamino-4,7,10-trioxatridecan-succinamic acid
  SITE 3: note = X is 5-chloro-L-tryptophan
  SITE 4: note = X is 4-(S)-Amino-L-proline with acetyl attached
  SITE 6: note = X is D-leucine
  SITE 8: note = X is 1-amino-isobutyric acid
  SITE 11: note = X is 2-aminoethane-1-thiol
  CROSSLNK 7..11: note = cysteine 2-aminoethane-1-thiol connected through 3-methylbenzylidene
DWXXEXCXLIX

SEQ ID NO: 113 - protein, 11 aa, synthetic construct
  MOD_RES 1: note = DOTA attached to D through 1,13-diamino-4,7,10-trioxatridecan-succinamic acid
  SITE 3: note = X is 5-chloro-L-tryptophan
  SITE 4: note = X is (2S,4R)-4-amino-1-pyrrolidin-2-carboxylic acid
  SITE 6: note = X is D-leucine
  SITE 8: note = X is 4-aminopiperidine-4-carboxylic acid
  SITE 11: note = X is 2-aminoethane-1-thiol
  CROSSLNK 7..11: note = cysteine 2-aminoethane-1-thiol through 2,6-lutidylidene
DWXXEXCXLIX

SEQ ID NO: 114 - protein, 11 aa, synthetic construct
  MOD_RES 1: note = DOTA attached to D through 1,13-diamino-4,7,10-trioxatridecan-succinamic acid
  SITE 3: note = X is 5-chloro-L-tryptophan
  SITE 4: note = X is (2S,4R)-4-amino-1-pyrrolidin-2-carboxylic acid
  SITE 6: note = X is D-leucine
  SITE 8: note = X is 4-aminopiperidine-4-carboxylic acid
  CROSSLNK 7..11: note = (S)-2,3-Diaminopropionic acid 2-aminoethane-1-thiol connected through 2,6-lutidylidene
DWXXEXXXLIX

SEQ ID NO: 115 - protein, 10 aa, synthetic construct
  MOD_RES 1: note = HO-succinyl attached to W
  SITE 2: note = X is 5-chloro-L-tryptophan
  SITE 3: note = X is 4-(R)-hydroxy-L-proline
  SITE 5: note = X is D-leucine
  SITE 7: note = X is 4-aminopiperidine-4-carboxylic acid
  MOD_RES 10: note = DOTA bearing K attached to C through 4-amino-1-carboxymethylpiperidine
  CROSSLNK 6..10: note = cysteine cysteine connected through 2,6-lutidylidene
WXXEXCXLIC

SEQ ID NO: 116 - protein, 10 aa, synthetic construct
  MOD_RES 1: note = HO-succinyl attached to W
  SITE 2: note = X is 5-chloro-L-tryptophan
  SITE 3: note = X is 4-(R)-hydroxy-L-proline
  SITE 5: note = X is D-leucine
  SITE 7: note = X is 4-amino-tetrahydropyran-4-carboxylic acid
  CROSSLNK 6..10: note = cysteine cysteine connected through 2,6-lutidylidene
  MOD_RES 10: note = DOTA bearing K attached to C through 4-amino-1-carboxymethylpiperidine
WXXEXCXLIC

SEQ ID NO: 117 - protein, 10 aa, synthetic construct
  MOD_RES 1: note = HO-succinyl attached to W
  SITE 2: note = X is 5-chloro-L-tryptophan
  SITE 3: note = X is 4-(R)-hydroxy-L-proline
  SITE 5: note = X is D-leucine
  SITE 7: note = X is 4-amino-tetrahydropyran-4-carboxylic acid
  MOD_RES 10: note = X is D-lysine with DOTA attached
  CROSSLNK 6..10: note = cysteine cysteine connected through 2,6-lutidylidene
WXXEXCXLIC

SEQ ID NO: 118 - protein, 10 aa, synthetic construct
  MOD_RES 1: note = HO-succinyl attached to W
  SITE 2: note = X is 5-chloro-L-tryptophan
  SITE 3: note = X is4-(R)-hydroxy-L-proline
  SITE 5: note = X is D-leucine
  SITE 7: note = X is 4-amino-tetrahydropyran-4-carboxylic acid
  MOD_RES 10: note = 1,5-diaminepentane with DOTA attached
  CROSSLNK 6..10: note = cysteine cysteine connected through 2,6-lutidylidene
WXXEXCXLIC

SEQ ID NO: 119 - protein, 10 aa, synthetic construct
  MOD_RES 1: note = HO-succinyl attached to W
  SITE 2: note = X is 5-chloro-L-tryptophan
  SITE 3: note = X is 4-(R)-hydroxy-L-proline
  SITE 5: note = X is D-leucine
  SITE 7: note = X is 4-amino-tetrahydropyran-4-carboxylic acid
  MOD_RES 10: note = DOTA attached through piperazine
  CROSSLNK 6..10: note = cysteine cysteine connected through 2,6-lutidylidene
WXXEXCXLIC

SEQ ID NO: 120 - protein, 11 aa, synthetic construct
  MOD_RES 1: note = DOTA attached to D through 1,13-diamino-4,7,10-trioxatridecan-succinamic acid
  SITE 3: note = X is 5-chloro-L-tryptophan
  SITE 6: note = X is D-leucine
  SITE 8: note = X is 4-aminopiperidine-4-carboxylic acid
  SITE 10: note = X is 2-aminoethane-1-thiol
  CROSSLNK 7..11: note = cysteine 2-aminoethane-1-thiol through 2,6-lutidylidene
DWXPEXCXLIX

SEQ ID NO: 121 - protein, 11 aa, synthetic construct
  MOD_RES 1: note = Polydisperse 2 kDa PEG
  SITE 3: note = X is 5-chloro-L-tryptophan
  SITE 4: note = X is 4-(R)-hydroxy-L-proline
  SITE 6: note = X is D-leucine
  SITE 8: note = X is 4-amino-tetrahydropyran-4-carboxylic acid
  MOD_RES 11: note = D-lysine with DOTA attached
  CROSSLNK 7..11: note = cysteine cysteine connected through 2,6-lutidylidene
DWXXEXCXLIC

SEQ ID NO: 122 - protein, 11 aa, synthetic construct
  MOD_RES 1: note = Acetyl attached to D
  SITE 3: note = X is 5-chloro-L-tryptophan
  SITE 4: note = X is (2S,4R)-4-amino-1-pyrrolidin-2-carboxylic acid with DOTA attached
  SITE 6: note = X is D-leucine
  SITE 8: note = X is 4-amino-tetrahydropyran-4-carboxylic acid
  CROSSLNK 7..11: note = cysteine cysteine connected through 2,6-lutidylidene
DWXXEXCXLIC

SEQ ID NO: 123 - protein, 11 aa, synthetic construct
  MOD_RES 1: note = Acetyl attached to D
  SITE 3: note = X is 5-chloro-L-tryptophan
  SITE 4: note = X is 4-(R)-hydroxy-L-proline
  SITE 6: note = X is D-leucine
  SITE 8: note = X is 4-aminopiperidine-4-carboxylic acid DOTA conjugate
  CROSSLNK 7..11: note = cysteine cysteine connected through 2,6-lutidylidene
DWXXEXCXLIC

SEQ ID NO: 124 - protein, 11 aa, synthetic construct
  MOD_RES 1: note = Acetyl attached to D
  LIPID 3: note = X is 5-chloro-L-tryptophan
  SITE 4: note = X is (2S,4R)-4-amino-1-pyrrolidin-2-carboxylic acid
  SITE 6: note = X is D-leucine
  SITE 8: note = X is 4-aminopiperidine-4-carboxylic acid with DOTA attached
  CROSSLNK 7..11: note = cysteine cysteine connected through 2,6-lutidylidene
DWXXEXCXLIC

SEQ ID NO: 125 - protein, 11 aa, synthetic construct
  MOD_RES 1: note = Acetyl attached to D
  SITE 3: note = X is 5-chloro-L-tryptophan
  SITE 4: note = X is 4-(R)-hydroxy-L-proline
  SITE 6: note = X is D-leucine
  SITE 8: note = X is 4-amino-tetrahydropyran-4-carboxylic acid
  CROSSLNK 7..11: note = cysteine cysteine connected through DOTA bearing mesitylene via 2-aminoethane-1-thiol
DWXXEXCXLIC

SEQ ID NO: 126 - protein, 11 aa, unidentified
  MOD_RES 1: note = DOTA attached to D
  SITE 3: note = X is 5-chloro-L-tryptophan
  SITE 4: note = X is 4-(R)-hydroxy-L-proline
  SITE 6: note = X is D-leucine
  SITE 8: note = X is 4-amino-tetrahydropyran-4-carboxylic acid
  CROSSLNK 7..11: note = cysteine cysteine connected through 2,6-lutidylidene
DWXXEXLXLIC

SEQ ID NO: 127 - protein, 12 aa, synthetic construct
  MOD_RES 1: note = DOTA attached to Y through 1,13-diamino-4,7,10-trioxatridecan-succinamic acid
  CROSSLNK 8..12: note = cysteine cysteine connected through 3-methylbenzylidene
YDWWPELCALIC

SEQ ID NO: 128 - protein, 12 aa, synthetic construct
  MOD_RES 1: note = Acetyl attached to Y
  CROSSLNK 8..12: note = cysteine cysteine connected through 3-methylbenzylidene
YDWWPELCALIC

SEQ ID NO: 129 - protein, 11 aa, synthetic construct
  MOD_RES 1: note = DOTA attached to D through 1,13-diamino-4,7,10-trioxatridecan-succinamic acid
  SITE 6: note = X is D-leucine
  SITE 8: note = X is 1-amino-isobutyric acid
  CROSSLNK 7..11: note = cysteine cysteine connected through 3-methylbenzylidene
DWWPEXCXLIC

SEQ ID NO: 130 - protein, 11 aa, synthetic construct
  MOD_RES 1: note = DOTA attached to D through 1,13-diamino-4,7,10-trioxatridecan-succinamic acid
  SITE 6: note = X is D-leucine
  SITE 8: note = X is 1-amino-isobutyric acid
  SITE 11: note = X is 2-aminoethane-1-thiol
  CROSSLNK 7..11: note = cysteine 2-aminoethane-1-thiol connected through 3-methylbenzylidene
DWWPEXCXLIX

SEQ ID NO: 131 - protein, 11 aa, synthetic construct
  SITE 1: note = X is N-methyl-L-aspartic acid
  MOD_RES 1: note = DOTA attached to N-methyl-L-aspartic acid through 1,13-diamino-4,7,10-trioxatridecan-succinamic acid
  SITE 6: note = X is D-leucine
  SITE 8: note = X is 1-amino-isobutyric acid
  SITE 11: note = X is 2-aminoethane-1-thiol
  CROSSLNK 7..11: note = cysteine 2-aminoethane-1-thiol connected through 3-methylbenzylidene
XWWPEXCXLIX

SEQ ID NO: 132 - protein, 11 aa, synthetic construct
  MOD_RES 1: note = Acetyl attached to D
  SITE 8: note = X is 1-amino-isobutyric acid
  CARBOHYD 11: note = DOTA bearing L-lysine attached to C through 1,13-diamino-4,7,10-trioxatridecan-succinamic acid
  CROSSLNK 7..11: note = cysteine cysteine connected through 3-methylbenzylidene
DWWPELCXLIC

SEQ ID NO: 133 - protein, 11 aa, synthetic construct
  MOD_RES 1: note = Acetyl attached to D
  SITE 6: note = X is L-neopentyl-glycine
  SITE 8: note = X is 1-amino-isobutyric acid
  MOD_RES 11: note = DOTA bearing L-lysine attached to C through 1,13-diamino-4,7,10-trioxatridecan-succinamic acid
  CROSSLNK 7..11: note = cysteine cysteine connected through 3-methylbenzylidene
DWWPEXCXLIC

SEQ ID NO: 134 - protein, 11 aa, synthetic construct
  MOD_RES 1: note = Acetyl attached to D
  SITE 8: note = X is 1-amino-isobutyric acid
  SITE 9: note = X is L-neopentyl-glycine
  CROSSLNK 7..11: note = cysteine cysteine connected through 3-methylbenzylidene
DWWPEXCXXIC

SEQ ID NO: 135 - protein, 11 aa, synthetic construct
  MOD_RES 1: note = Acetyl attached to D
  SITE 7: note = X is 1-amino-isobutyric acid
  SITE 9: note = X is L-cyclo-hexyl-alanine
  MOD_RES 11: note = DOTA bearing L-lysine attached to C through 1,13-diamino-4,7,10-trioxatridecan-succinamic acid
  CROSSLNK 7..11: note = cysteine cysteine connected through 3-methylbenzylidene
DWWPELCXXIC

SEQ ID NO: 136 - protein, 11 aa, synthetic construct
  MOD_RES 1: note = 3-phenyl-propionic acid attached to D
  SITE 6: note = X is D-leucine
  SITE 8: note = X is 1-amino-isobutyric acid
  MOD_RES 11: note = DOTA bearing L-lysine attached to C through 1,13-diamino-4,7,10-trioxatridecan-succinamic acid
  CROSSLNK 7..11: note = cysteine cysteine connected through 3-methylbenzylidene
DWWPEXCXLIC

SEQ ID NO: 137 - protein, 11 aa, synthetic construct
  MOD_RES 1: note = 4-fluorophenyl propionic acid attached to D
  SITE 6: note = X is D-leucine
  SITE 8: note = X is 1-amino-isobutyric acid
  MOD_RES 11: note = DOTA bearing L-lysine attached to C through 1,13-diamino-4,7,10-trioxatridecan-succinamic acid
  CROSSLNK 7..11: note = cysteine cysteine connected through 3-methylbenzylidene
DWWPEXCXLIC

SEQ ID NO: 138 - protein, 11 aa, synthetic construct
  MOD_RES 1: note = 4-hydroxyphenyl propionic acid attached to D
  SITE 6: note = X is D-leucine
  SITE 8: note = 1-amino-isobutyric acid
  MOD_RES 11: note = DOTA bearing L-lysine attached to C through 1,13-diamino-4,7,10-trioxatridecan-succinamic acid
  CROSSLNK 7..11: note = cysteine cysteine connected through 3-methylbenzylidene
DWWPEXCXLIC

SEQ ID NO: 139 - protein, 11 aa, synthetic construct
  MOD_RES 1: note = Acetyl attached to D
  SITE 3: note = X is 3-(1-naphthyl)-L-alanine
  SITE 6: note = X is D-leucine
  MOD_RES 11: note = DOTA bearing L-lysine attached to C through 1,13-diamino-4,7,10-trioxatridecan-succinamic acid
  CROSSLNK 7..11: note = cysteine cysteine connected through 3-methylbenzylidene
DWXPEXCALVC

SEQ ID NO: 140 - protein, 11 aa, synthetic construct
  MOD_RES 1: note = Acetyl attached to D
  SITE 3: note = X is 3-(1-naphthyl)-L-alanine
  SITE 6: note = X is D-leucine
  SITE 8: note = X is diethylgylcine
  MOD_RES 11: note = DOTA bearing L-lysine attached to C through 1,13-diamino-4,7,10-trioxatridecan-succinamic acid
  CROSSLNK 7..11: note = cysteine cysteine connected through 3-methylbenzylidene
DWXPEXCXLIC

SEQ ID NO: 141 - protein, 11 aa, synthetic construct
  MOD_RES 1: note = Acetyl attached to D
  SITE 3: note = X is 6-fluoro-L-tryptophan
  SITE 6: note = X is D-leucine
  SITE 8: note = X is 1-amino-isobutyric acid
  MOD_RES 11: note = DOTA bearing L-lysine attached to C through 1,13-diamino-4,7,10-trioxatridecan-succinamic acid
  CROSSLNK 7..11: note = cysteine cysteine connected through 3-methylbenzylidene
DWXPEXCXLIC

SEQ ID NO: 142 - protein, 11 aa, synthetic construct
  MOD_RES 1: note = DOTA attached to D through 1,13-diamino-4,7,10-trioxatridecan-succinamic acid
  SITE 2: note = X is 6-chloro-L-tryptophan
  SITE 6: note = X is D-leucine
  SITE 8: note = X is 1-amino-isobutyric acid
  SITE 11: note = X is 2-aminoethane-1-thiol
  CROSSLNK 7..11: note = cysteine 2-aminoethane-1-thiol connected through 3-methylbenzylidene
DXWPEXCXLIX

SEQ ID NO: 143 - protein, 10 aa, synthetic construct
  MOD_RES 1: note = HO-succinyl attached to W
  SITE 3: note = X is 4-(R)-hydroxy-L-proline
  SITE 2: note = X is 5-chloro-L-tryptophan
  SITE 5: note = X is D-leucine
  SITE 7: note = X is 4-aminopiperidine-4-carboxylic acid conjugated to polydisperse 10 kDa PEG
  MOD_RES 10: note = DOTA bearing K attached to C through 4-amino-1-carboxymethylpiperidine
  CROSSLNK 6..10: note = cysteine cysteine connected through 2,6-lutidylidene
WXXEXCXLIC

SEQ ID NO: 144 - protein, 10 aa, synthetic construct
  MOD_RES 1: note = HO-succinyl attached to W
  SITE 2: note = X is 5-chloro-L-tryptophan
  SITE 3: note = X is 4-(R)-hydroxy-L-proline
  SITE 5: note = X isD-leucine
  SITE 7: note = X is 4-aminopiperidine-4-carboxylic acid conjugated to polydisperse 10 kDa PEG
  MOD_RES 10: note = DOTA bearing K attached to C through 4-amino-1-carboxymethylpiperidine
  CROSSLNK 6..10: note = cysteine cysteine connected through 2,6-lutidylidene
WXXEXCXLIC

SEQ ID NO: 145 - protein, 10 aa, synthetic construct
  MOD_RES 1: note = HO-succinyl attached to W
  SITE 2: note = X is 5-chloro-L-tryptophan
  SITE 3: note = X is 4-(R)-hydroxy-L-proline
  SITE 5: note = X is D-leucine
  SITE 7: note = X is 4-amino-tetrahydropyran-4-carboxylic acid
  MOD_RES 10: note = D-lysine conjugated to polydisperse 2 kDa PEG and to L-lysine bearing DOTA with the D-lysine being attached to C through 4-amino-1-carboxymethylpiperidine
  CROSSLNK 6..10: note = cysteine cysteine connected through 2,6-lutidylidene
WXXEXCXLIC

SEQ ID NO: 146 - protein, 11 aa, synthetic construct
  MOD_RES 1: note = Acetyl attached to D
  SITE 3: note = X is 5-chloro-L-tryptophan
  SITE 4: note = X is 4-(R)-hydroxy-L-proline
  SITE 6: note = X is D-leucine
  SITE 8: note = 4-amino-tetrahydropyran-4-carboxylic acid
  CROSSLNK 7..11: note = cysteine cysteine connected through DOTA bearing mesitylene via piperazine
DWXXEXCXLIC

SEQ ID NO: 147 - protein, 11 aa, synthetic construct
  MOD_RES 1: note = 1-amino-1-deoxy-D-glucitol attached through glutaric acid to D
  SITE 3: note = X is 5-chloro-L-tryptophan
  SITE 4: note = X is 4-(R)-hydroxy-L-proline
  SITE 6: note = X is D-leucine
  SITE 8: note = X is 4-aminopiperidine-4-carboxylic acid with InDOTA attached
  SITE 11: note = X is 2-aminoethane-1-thiol
  CROSSLNK 7..11: note = cysteine 2-aminoethane-1-thiol through 2,6-lutidylidene
DWXXEXCXLIX

SEQ ID NO: 148 - protein, 11 aa, synthetic construct
  MOD_RES 1: note = 1-amino-1-deoxy-D-glucitol attached through glutaric acid to D
  SITE 3: note = 5-chloro-L-tryptophan
  SITE 4: note = X is 4-(R)-hydroxy-L-proline
  SITE 6: note = X is D-leucine
  SITE 8: note = 4-aminopiperidine-4-carboxylic acid with ZnDOTA attached
  SITE 11: note = X is 2-aminoethane-1-thiol
  CROSSLNK 7..11: note = cysteine 2-aminoethane-1-thiol through 2,6-lutidylidene
DWXXEXCXLIX

SEQ ID NO: 149 - protein, 11 aa, synthetic construct
  MOD_RES 1: note = InDOTA attached to D through 1,13-diamino-4,7,10-trioxatridecan-succinamic acid
  SITE 3: note = X is 5-chloro-L-tryptophan
  SITE 6: note = X is D-leucine
  SITE 8: note = X is 1-amino-isobutyric acid
  SITE 11: note = X is 2-aminoethane-1-thiol
  CROSSLNK 7..11: note = cysteine 2-aminoethane-1-thiol connected through 3-methylbenzylidene
DWXPEXCXLIX

SEQ ID NO: 150 - protein, 11 aa, synthetic construct
  MOD_RES 1: note = InDOTA attached to D through 1,13-diamino-4,7,10-trioxatridecan-succinamic acid
  SITE 3: note = X is 5-chloro-L-tryptophan
  SITE 6: note = X is D-leucine
  SITE 8: note = X is alpha-methyl-L-serine
  SITE 11: note = X is 2-aminoethane-1-thiol
  CROSSLNK 7..11: note = cysteine 2-aminoethane-1-thiol connected through 3-methylbenzylidene
DWXPEXCXLIX

SEQ ID NO: 151 - protein, 11 aa, synthetic construct
  MOD_RES 1: note = InDOTA attached to D through 1,13-diamino-4,7,10-trioxatridecan-succinamic acid
  SITE 3: note = X is 5-chloro-L-tryptophan
  SITE 6: note = X is D-leucine
  SITE 8: note = X is 4-amino-tetrahydropyran-4-carboxylic acid
  SITE 11: note = X is 2-aminoethane-1-thiol
  CROSSLNK 7..11: note = cysteine 2-aminoethane-1-thiol connected through 3-methylbenzylidene
DWXPEXCXLIX

SEQ ID NO: 152 - protein, 11 aa, synthetic construct
  MOD_RES 1: note = InDOTA attached to D through 1,13-diamino-4,7,10-trioxatridecan-succinamic acid
  SITE 3: note = X is 5-chloro-L-tryptophan
  SITE 4: note = X is 4-(R)-hydroxy-L-proline
  SITE 6: note = X is D-leucine
  SITE 8: note = X is 1-amino-isobutyric acid
  SITE 11: note = X is 2-aminoethane-1-thiol
  CROSSLNK 7..11: note = cysteine 2-aminoethane-1-thiol connected through 3-methylbenzylidene
DWXXEXCXLIX

SEQ ID NO: 153 - protein, 11 aa, synthetic construct
  MOD_RES 1: note = InDOTA attached to D through 1,13-diamino-4,7,10-trioxatridecan-succinamic acid
  SITE 3: note = X is 5-chloro-L-tryptophan
  SITE 4: note = X is N-methyl-glycine
  SITE 6: note = X is D-leucine
  SITE 8: note = X is 1-amino-isobutyric acid
  SITE 11: note = X is 2-aminoethane-1-thiol
  CROSSLNK 7..11: note = cysteine 2-aminoethane-1-thiol connected through 3-methylbenzylidene
DWXXEXCXLIX

SEQ ID NO: 154 - protein, 11 aa, synthetic construct
  MOD_RES 1: note = InDOTA attached to D through 4-amino-1-carboxymethylpiperidine
  SITE 3: note = X is 5-chloro-L-tryptophan
  SITE 6: note = X is D-leucine
  SITE 8: note = X is 1-amino-isobutyric acid
  SITE 11: note = X is 2-aminoethane-1-thiol
  CROSSLNK 7..11: note = cysteine 2-aminoethane-1-thiol connected through 3-methylbenzylidene
DWXPEXCXLIX

SEQ ID NO: 155 - protein, 11 aa, synthetic construct
  MOD_RES 1: note = InDOTA attached to D through 1,13-diamino-4,7,10-trioxatridecan-succinamic acid
  SITE 3: note = X is 5-chloro-L-tryptophan
  SITE 6: note = X is D-leucine
  SITE 8: note = X is 1-amino-isobutyric acid
  SITE 11: note = X is 2-aminoethane-1-thiol
  CROSSLNK 7..11: note = cysteine 2-aminoethane-1-thiol through 2,6-lutidylidene
DWXPEXCXLIX

SEQ ID NO: 156 - protein, 11 aa, synthetic construct
  MOD_RES 1: note = InDOTA attached to D through 4-amino-1-carboxymethylpiperidine
  SITE 3: note = X is 5-chloro-L-tryptophan
  SITE 4: note = X is 4-(R)-hydroxy-L-proline
  SITE 6: note = X is D-leucine
  SITE 8: note = X is 4-amino-tetrahydropyran-4-carboxylic acid
  SITE 11: note = X is 2-aminoethane-1-thiol
  CROSSLNK 7..11: note = cysteine 2-aminoethane-1-thiol through 2,6-lutidylidene
DWXXEXCXLIX

SEQ ID NO: 157 - protein, 11 aa, synthetic construct
  MOD_RES 1: note = Acetyl attached to D
  SITE 3: note = X is 5-chloro-L-tryptophan
  SITE 6: note = X is D-leucine
  SITE 8: note = X is 1-amino-isobutyric acid
  MOD_RES 11: note = LuDOTA bearing L-lysine attached to C through 1,13-diamino-4,7,10-trioxatridecan-succinamic acid
  CROSSLNK 7..11: note = cysteine cysteine connected through 3-methylbenzylidene
DWXPEXCXLIC

SEQ ID NO: 158 - protein, 11 aa, synthetic construct
  MOD_RES 1: note = LuDOTA attached to D through 1,13-diamino-4,7,10-trioxatridecan-succinamic acid
  SITE 3: note = X is 5-chloro-L-tryptophan
  SITE 6: note = X is D-leucine
  SITE 8: note = X is alpha-methyl-L-serine
  SITE 11: note = X is 2-aminoethane-1-thiol
  CROSSLNK 7..11: note = cysteine 2-aminoethane-1-thiol connected through 3-methylbenzylidene
DWXPEXCXLIX

SEQ ID NO: 159 - protein, 11 aa, synthetic construct
  MOD_RES 1: note = LuDOTA attached to D through 1,13-diamino-4,7,10-trioxatridecan-succinamic acid
  SITE 3: note = X is 5-chloro-L-tryptophan
  SITE 6: note = X is D-leucine
  SITE 8: note = X is 4-amino-tetrahydropyran-4-carboxylic acid
  SITE 11: note = X is 2-aminoethane-1-thiol
  CROSSLNK 7..11: note = cysteine 2-aminoethane-1-thiol connected through 3-methylbenzylidene
DWXPEXCXLIX

SEQ ID NO: 160 - protein, 11 aa, synthetic construct
  MOD_RES 1: note = LuDOTA attached to D through 1,13-diamino-4,7,10-trioxatridecan-succinamic acid
  SITE 3: note = X is 5-chloro-L-tryptophan
  SITE 4: note = X is 4-(R)-hydroxy-L-proline
  SITE 6: note = X is D-leucine
  SITE 8: note = X is 1-amino-isobutyric acid
  SITE 11: note = X is 2-aminoethane-1-thiol
  CROSSLNK 7..11: note = cysteine 2-aminoethane-1-thiol connected through 3-methylbenzylidene
DWXXEXCXLIX

SEQ ID NO: 161 - protein, 11 aa, synthetic construct
  MOD_RES 1: note = LuDOTA attached to D through 1,13-diamino-4,7,10-trioxatridecan-succinamic acid
  SITE 3: note = X is 5-chloro-L-tryptophan
  SITE 4: note = X is N-methyl-glycine
  SITE 6: note = X is D-leucine
  SITE 8: note = X is 1-amino-isobutyric acid
  SITE 11: note = X is 2-aminoethane-1-thiol
  CROSSLNK 7..11: note = cysteine 2-aminoethane-1-thiol connected through 3-methylbenzylidene
DWXXEXCXLIX

SEQ ID NO: 162 - protein, 11 aa, synthetic construct
  MOD_RES 1: note = LuDOTA attached to D through 4-amino-1-carboxymethylpiperidine
  SITE 3: note = X is 5-chloro-L-tryptophan
  SITE 6: note = X is D-leucine
  SITE 8: note = X is 1-amino-isobutyric acid
  SITE 11: note = X is 2-aminoethane-1-thiol
  CROSSLNK 7..11: note = cysteine 2-aminoethane-1-thiol connected through 3-methylbenzylidene
DWXPEXCXLIX

SEQ ID NO: 163 - protein, 11 aa, synthetic construct
  MOD_RES 1: note = LuDOTA attached to D through 4-amino-1-carboxymethylpiperidine
  SITE 3: note = X is 5-chloro-L-tryptophan
  SITE 4: note = X is 4-(R)-hydroxy-L-proline
  SITE 6: note = X is D-leucine
  SITE 8: note = X is 4-amino-tetrahydropyran-4-carboxylic acid
  SITE 11: note = X is 2-aminoethane-1-thiol
  CROSSLNK 7..11: note = cysteine 2-aminoethane-1-thiol through 2,6-lutidylidene
DWXXEXCXLIX

SEQ ID NO: 164 - protein, 11 aa, synthetic construct
  MOD_RES 1: note = LuDOTA attached to D through 1,13-diamino-4,7,10-trioxatridecan-succinamic acid
  SITE 3: note = X is 5-chloro-L-tryptophan
  SITE 6: note = X is D-leucine
  SITE 8: note = X is 1-amino-isobutyric acid
  SITE 11: note = X is 2-aminoethane-1-thiol
  CROSSLNK 7..11: note = cysteine 2-aminoethane-1-thiol through 2,6-lutidylidene
DWXPEXCXLIX

SEQ ID NO: 165 - protein, 11 aa, synthetic construct
  SITE 3: note = X is 5-chloro-L-tryptophan
  SITE 6: note = X is D-leucine
  SITE 8: note = X is 1-amino-isobutyric acid
  SITE 11: note = X is 2-aminoethane-1-thiol
  CROSSLNK 7..11: note = cysteine 2-aminoethane-1-thiol through 2,6-lutidylidene
DWXPEXCXLIC

SEQ ID NO: 166 - protein, 11 aa, synthetic construct
  SITE 3: note = X is 3-(1-naphthyl)-L-alanine
  SITE 6: note = X is D-leucine
  SITE 8: note = X is 1-amino-isobutyric acid
  CROSSLNK 7..11: note = cysteine cysteine connected through 3-methylbenzylidene
DWXPEXCXLIC

SEQ ID NO: 167 - protein, 11 aa, synthetic construct
  SITE 3: note = X is 3-(1-naphthyl)-L-alanine
  SITE 6: note = X is D-leucine
  SITE 8: note = X is 1-Amino-1-cyclohexan-carboxylic acid
  CROSSLNK 7..11: note = cysteine cysteine connected through 3-methylbenzylidene
DWXPEXCXLIC

SEQ ID NO: 168 - protein, 11 aa, synthetic construct
  SITE 3: note = X is 5-chloro-L-tryptophan
  SITE 6: note = X is D-leucine
  SITE 8: note = X is 1-amino-isobutyric acid
  SITE 11: note = X is 2-aminoethane-1-thiol
  CROSSLNK 7..11: note = cysteine 2-aminoethane-1-thiol connected through 3-methylbenzylidene
DWXPEXCXLIX

SEQ ID NO: 169 - protein, 10 aa, synthetic construct
  SITE 2: note = X is -chloro-L-tryptophan
  SITE 3: note = X is 4-(R)-hydroxy-L-proline
  SITE 5: note = X is D-leucine
  SITE 7: note = X is 4-amino-tetrahydropyran-4-carboxylic acid
  CROSSLNK 6..10: note = cysteine cysteine connected through 2,6-lutidylidene
WXXEXCXLIC

SEQ ID NO: 170 - protein, 10 aa, synthetic construct
  SITE 2: note = X is 5-chloro-L-tryptophan
  SITE 3: note = X is 4-(R)-hydroxy-L-proline
  SITE 5: note = X is D-leucine
  SITE 7: note = X is 4-aminopiperidine-4-carboxylic acid InDOTA conjugate
  SITE 10: note = X is 2-aminoethane-1-thiol
  CROSSLNK 6..10: note = cysteine 2-aminoethane-1-thiol through 2,6-lutidylidene
WXXEXCXLIX

SEQ ID NO: 171 - protein, 10 aa, synthetic construct
  SITE 2: note = X is 5-chloro-L-tryptophan
  SITE 3: note = X is 4-(R)-hydroxy-L-proline
  SITE 5: note = X is D-leucine
  SITE 7: note = X is 4-amino-tetrahydropyran-4-carboxylic acid
  CROSSLNK 6..10: note = cysteine cysteine connected through 2,6-lutidylidene
WXXEXCXLIC

SEQ ID NO: 172 - protein, 10 aa, synthetic construct
  SITE 2: note = X is 5-chloro-L-tryptophan
  SITE 3: note = X is 4-(R)-hydroxy-L-proline
  SITE 5: note = X is D-leucine
  SITE 7: note = 4-aminopiperidine-4-carboxylic acid LuDOTA conjugate
  SITE 10: note = X is 2-aminoethane-1-thiol
  CROSSLNK 6..10: note = cysteine 2-aminoethane-1-thiol through 2,6-lutidylidene
WXXEXCXLIX

SEQ ID NO: 173 - protein, 11 aa, synthetic construct
  SITE 3: note = X is 3-(1-naphthyl)-L-alanine
  SITE 6: note = X is D-leucine
  SITE 8: note = X is 1-amino-isobutyric acid
  CROSSLNK 7..11: note = cysteine cysteine connected through 3-methylbenzylidene
DWXPEXCXLIC

SEQ ID NO: 174 - protein, 11 aa, synthetic construct
  SITE 3: note = X is 3-(1-naphthyl)-L-alanine
  SITE 6: note = X is D-leucine
  SITE 8: note = X is 1-Amino-1-cyclohexan-carboxylic acid
  CROSSLNK 7..11: note = cysteine cysteine connected through 3-methylbenzylidene
DWXPEXCXLIC

SEQ ID NO: 175 - protein, 11 aa, synthetic construct
  SITE 3: note = X is 5-chloro-L-tryptophan
  SITE 4: note = X is 4-(R)-hydroxy-L-proline
  SITE 6: note = X is D-leucine
  SITE 8: note = X is 4-amino-tetrahydropyran-4-carboxylic acid
  CROSSLNK 7..11: note = cysteine cysteine connected through 2,6-lutidylidene
DWXXEXCXLIC

SEQ ID NO: 176 - protein, 11 aa, synthetic construct
  SITE 3: note = X is 5-chloro-L-tryptophan
  SITE 4: note = X is (2S,4R)-4-amino-1-pyrrolidin-2-carboxylic acid
  SITE 6: note = X is D-leucine
  SITE 8: note = X is 4-amino-tetrahydropyran-4-carboxylic acid
  CROSSLNK 7..11: note = cysteine cysteine connected through 2,6-lutidylidene
DWXXEXCXLIC

SEQ ID NO: 177 - protein, 11 aa, synthetic construct
  SITE 3: note = X is 5-chloro-L-tryptophan
  SITE 4: note = X is N-glutamic acid, N carboxyethyl glycine with 1-amino-1-deoxy-D-glucitol attached
  SITE 6: note = X is D-leucine
  SITE 8: note = X is 4-amino-tetrahydropyran-4-carboxylic acid
  CROSSLNK 7..11: note = cysteine cysteine connected through 2,6-lutidylidene
DWXXEXCXLIC

SEQ ID NO: 178 - protein, 11 aa, synthetic construct
  SITE 3: note = X is 5-chloro-L-tryptophan
  SITE 4: note = X is 4-(R)-hydroxy-L-proline
  SITE 6: note = X is D-leucine
  SITE 8: note = X is 4-amino-tetrahydropyran-4-carboxylic acid
  CROSSLNK 7..11: note = cysteine cysteine connected through 2,6-lutidylidene
DWXXEXCXLIC

SEQ ID NO: 179 - protein, 11 aa, synthetic construct
  SITE 3: note = X is 5-chloro-L-tryptophan
  SITE 4: note = X is 4-(R)-hydroxy-L-proline
  SITE 6: note = X is D-leucine
  SITE 8: note = X is 4-amino-tetrahydropyran-4-carboxylic acid
  CROSSLNK 7..11: note = cysteine cysteine connected through 2,6-lutidylidene
DWXXEXCXLIC

SEQ ID NO: 180 - protein, 11 aa, synthetic construct
  SITE 1: note = X is L-cysteic acid
  SITE 3: note = X is 5-chloro-L-tryptophan
  SITE 4: note = X is 4-(R)-hydroxy-L-proline
  SITE 6: note = X is D-leucine
  SITE 8: note = X is 1-amino-isobutyric acid
  SITE 11: note = X is 2-aminoethane-1-thiol
  CROSSLNK 7..11: note = cysteine 2-aminoethane-1-thiol through 2,6-lutidylidene
XWXXEXCXLIX

SEQ ID NO: 181 - protein, 11 aa, synthetic construct
  SITE 3: note = X is 5-chloro-L-tryptophan
  SITE 4: note = X is 4-(R)-hydroxy-L-proline
  SITE 6: note = X is D-leucine
  SITE 8: note = X is 4-amino-tetrahydropyran-4-carboxylic acid
  CROSSLNK 7..11: note = cysteine cysteine connected through 2,6-lutidylidene
DWXXEXCXLIC

SEQ ID NO: 182 - protein, 11 aa, synthetic construct
  SITE 3: note = X is 5-chloro-L-tryptophan
  SITE 4: note = X is 4-(R)-hydroxy-L-proline
  SITE 6: note = D is D-leucine
  SITE 8: note = X is 4-amino-tetrahydropyran-4-carboxylic acid
  CROSSLNK 7..11: note = cysteine cysteine connected through 2,6-lutidylidene
DWXXEXCXLIC

SEQ ID NO: 183 - protein, 11 aa, synthetic construct
  SITE 3: note = X is 5-chloro-L-tryptophan
  SITE 4: note = X is 4-(R)-hydroxy-L-proline
  SITE 6: note = X is D-leucine
  SITE 8: note = X is 4-amino-tetrahydropyran-4-carboxylic acid
  SITE 11: note = X is L-cysteinol
  CROSSLNK 7..11: note = cysteine L-cysteinol connected through 2,6-lutidylidene
DWXXEXCXLIX

SEQ ID NO: 184 - protein, 11 aa, synthetic construct
  SITE 3: note = X is 5-chloro-L-tryptophan
  SITE 4: note = X is N-methyl-glycine
  SITE 6: note = is D-leucine
  SITE 8: note = X is 4-amino-tetrahydropyran-4-carboxylic acid
  CROSSLNK 7..11: note = cysteine cysteine connected through 2,6-lutidylidene
DWXXEXCXLIC

SEQ ID NO: 185 - protein, 11 aa, synthetic construct
  SITE 3: note = X is 5-chloro-L-tryptophan
  SITE 4: note = X is N-leucine
  SITE 6: note = X is D-leucine
  SITE 8: note = X is 4-amino-tetrahydropyran-4-carboxylic acid
  CROSSLNK 7..11: note = cysteine cysteine connected through 2,6-lutidylidene
DWXXEXCXLIC

SEQ ID NO: 186 - protein, 11 aa, synthetic construct
  SITE 3: note = X is 5-chloro-L-tryptophan
  SITE 4: note = X is N-phenylalanine
  SITE 6: note = X is D-leucine
  SITE 8: note = X is 4-amino-tetrahydropyran-4-carboxylic acid
  CROSSLNK 7..11: note = cysteine cysteine connected through 2,6-lutidylidene
DWXXEXCXLIC

SEQ ID NO: 187 - protein, 11 aa, synthetic construct
  SITE 3: note = X is 5-chloro-L-tryptophan
  SITE 4: note = X is N-lysine
  SITE 6: note = X is D-leucine
  SITE 8: note = X is 4-amino-tetrahydropyran-4-carboxylic acid
  CROSSLNK 7..11: note = cysteine cysteine connected through 2,6-lutidylidene
DWXXEXCXLIC

SEQ ID NO: 188 - protein, 11 aa, synthetic construct
  SITE 3: note = X is 5-chloro-L-tryptophan
  SITE 4: note = X is 4-(R)-fluoro-L-proline
  SITE 6: note = X is D-leucine
  SITE 8: note = X is 4-amino-tetrahydropyran-4-carboxylic acid
  CROSSLNK 7..11: note = cysteine cysteine connected through 2,6-lutidylidene
DWXXEXCXLIC

SEQ ID NO: 189 - protein, 11 aa, synthetic construct
  SITE 3: note = X is 5-chloro-L-tryptophan
  SITE 4: note = X is 4-(R)-hydroxy-L-proline
  SITE 6: note = X is D-leucine
  SITE 8: note = X is 4-amino-tetrahydropyran-4-carboxylic acid
  CROSSLNK 7..11: note = cysteine cysteine connected through 2,6-lutidylidene
SWXXEXCXLIC

SEQ ID NO: 190 - protein, 11 aa, synthetic construct
  SITE 1: note = X is L-cysteic acid
  SITE 3: note = X is 5-chloro-L-tryptophan
  SITE 4: note = X is 4-(R)-hydroxy-L-proline
  SITE 6: note = X is D-leucine
  SITE 8: note = X is 4-amino-tetrahydropyran-4-carboxylic acid
  CROSSLNK 7..11: note = cysteine cysteine connected through 2,6-lutidylidene
XWXXEXLXLIC

SEQ ID NO: 191 - protein, 11 aa, synthetic construct
  SITE 3: note = X is 5-chloro-L-tryptophan
  SITE 4: note = X is 4-(R)-hydroxy-L-proline
  SITE 6: note = X is D-leucine
  SITE 8: note = X is 4-amino-tetrahydropyran-4-carboxylic acid
  CROSSLNK 7..11: note = cysteine cysteine connected through 2,6-lutidylidene
NWXXEXCXLIC

SEQ ID NO: 192 - protein, 11 aa, synthetic construct
  SITE 3: note = X is 5-chloro-L-tryptophan
  SITE 4: note = X is 4-(R)-hydroxy-L-proline
  SITE 6: note = X is D-leucine
  SITE 8: note = X is 4-amino-tetrahydropyran-4-carboxylic acid
  CROSSLNK 7..11: note = cysteine cysteine connected through 2,6-lutidylidene
EWXXEXCXLIC

SEQ ID NO: 193 - protein, 11 aa, synthetic construct
  SITE 3: note = X is 5-chloro-L-tryptophan
  SITE 4: note = X is 4-(R)-hydroxy-L-proline
  SITE 6: note = X is D-leucine
  SITE 8: note = X is 4-amino-tetrahydropyran-4-carboxylic acid
  CROSSLNK 7..11: note = cysteine cysteine connected through 2,6-lutidylidene
DWXXDXCXLIC

SEQ ID NO: 194 - protein, 11 aa, synthetic construct
  SITE 3: note = X is 5-chloro-L-tryptophan
  SITE 4: note = 4-(R)-hydroxy-L-proline
  SITE 6: note = X is D-leucine
  SITE 8: note = X is 4-amino-tetrahydropyran-4-carboxylic acid
  CROSSLNK 7..11: note = cysteine cysteine connected through 2,6-lutidylidene
DWXXQXCXLIC

SEQ ID NO: 195 - protein, 11 aa, synthetic construct
  SITE 3: note = X is 5-chloro-L-tryptophan
  SITE 4: note = X is 4-(R)-hydroxy-L-proline
  SITE 5: note = X is L-homoserine
  SITE 6: note = X is D-leucine
  SITE 8: note = X is 4-amino-tetrahydropyran-4-carboxylic acid
  CROSSLNK 7..11: note = cysteine cysteine connected through 2,6-lutidylidene
DWXXXXCXLIC

SEQ ID NO: 196 - protein, 11 aa, synthetic construct
  SITE 1: note = X is L-homoserine
  SITE 3: note = X is 5-chloro-L-tryptophan
  SITE 4: note = X is 4-(R)-hydroxy-L-proline
  SITE 6: note = X is D-leucine
  SITE 8: note = X is 4-amino-tetrahydropyran-4-carboxylic acid
  CROSSLNK 7..11: note = cysteine cysteine connected through 2,6-lutidylidene
XWXXEXCXLIC

SEQ ID NO: 197 - protein, 11 aa, synthetic construct
  SITE 3: note = X is 5-chloro-L-tryptophan
  SITE 4: note = X is N-glutamic acid
  SITE 6: note = X is D-leucine
  SITE 8: note = X is 4-amino-tetrahydropyran-4-carboxylic acid
  CROSSLNK 7..11: note = cysteine cysteine connected through 2,6-lutidylidene
DWXXEXCXLIC

SEQ ID NO: 198 - protein, 11 aa, synthetic construct
  SITE 1: note = X is L-homoserine
  SITE 3: note = X is 5-bromo-L-tryptophan
  SITE 4: note = X is 4-(R)-hydroxy-L-proline
  SITE 6: note = X is D-leucine
  SITE 8: note = X is 4-amino-tetrahydropyran-4-carboxylic acid
  CROSSLNK 7..11: note = cysteine cysteine connected through 2,6-lutidylidene
XWXXEXCXLIC

SEQ ID NO: 199 - protein, 10 aa, synthetic construct
  SITE 2: note = X is 5-chloro-L-tryptophan
  SITE 5: note = X is D-leucine
  SITE 7: note = X is 1-amino-isobutyric acid
  CROSSLNK 6..10: note = cysteine cysteine connected through 3-methylbenzylidene
WXPEXCXLIC

SEQ ID NO: 200 - protein, 10 aa, synthetic construct
  SITE 2: note = X is 5-chloro-L-tryptophan
  SITE 5: note = X is D-leucine
  SITE 7: note = X is 4-amino-tetrahydropyran-4-carboxylic acid
  CROSSLNK 6..10: note = cysteine cysteine connected through 3-methylbenzylidene
WXPEXCXLIC

SEQ ID NO: 201 - protein, 11 aa, synthetic construct
  SITE 3: note = X is 5-chloro-L-tryptophan
  SITE 4: note = X is 4-(R)-hydroxy-L-proline
  SITE 6: note = X is D-leucine
  SITE 7: note = X is L-homocysteine
  SITE 8: note = X is 4-amino-tetrahydropyran-4-carboxylic acid
  CROSSLNK 7..11: note = L-homocysteine cysteine connected through 2,6-lutidylidene
DWXXEXXXLIC

SEQ ID NO: 202 - protein, 11 aa, synthetic construct
  SITE 3: note = X is 5-chloro-L-tryptophan
  SITE 4: note = X is 4-(R)-hydroxy-L-proline
  SITE 6: note = X is D-leucine
  SITE 8: note = X is 4-amino-tetrahydropyran-4-carboxylic acid
  SITE 11: note = X is L-homocysteine
  CROSSLNK 7..11: note = cysteine L-homocysteine connected through 2,6-lutidylidene
DWXXEXCXLIX

SEQ ID NO: 203 - protein, 11 aa, synthetic construct
  SITE 3: note = X is 5-chloro-L-tryptophan
  SITE 4: note = X is 4-(R)-hydroxy-L-proline
  SITE 6: note = X is D-leucine
  SITE 7: note = X is L-homocysteine
  SITE 8: note = X is 4-amino-tetrahydropyran-4-carboxylic acid
  SITE 11: note = X is L-homocysteine
  CROSSLNK 7..11: note = L-homocysteine L-homocysteine connected through 2,6-lutidylidene
DWXXEXXXLIX

SEQ ID NO: 204 - protein, 11 aa, synthetic construct
  SITE 3: note = X is 5-chloro-L-tryptophan
  SITE 4: note = X is 4-(R)-hydroxy-L-proline
  SITE 6: note = X is D-leucine
  SITE 8: note = X is 1-amino-1-cyclopentane carboxylic acid
  CROSSLNK 7..11: note = cysteine cysteine connected through 2,6-lutidylidene
DWXXEXCXLIC

SEQ ID NO: 205 - protein, 11 aa, synthetic construct
  SITE 3: note = X is 5-chloro-L-tryptophan
  SITE 4: note = X is 4-(R)-hydroxy-L-proline
  SITE 6: note = X is D-leucine
  SITE 8: note = X is 4-amino-tetrahydropyran-4-carboxylic acid
  CROSSLNK 7..11: note = cysteine cysteine connected through 2,6-lutidylidene
QWXXEXCXLIC

SEQ ID NO: 206 - protein, 11 aa, synthetic construct
  SITE 3: note = X is 5-chloro-L-tryptophan
  SITE 4: note = X is 4-(R)-hydroxy-L-proline
  SITE 6: note = X is D-leucine
  SITE 8: note = X is 4-amino-tetrahydropyran-4-carboxylic acid
  CROSSLNK 7..11: note = cysteine cysteine connected through 2,6-lutidylidene
DWXXNXCXLIC

SEQ ID NO: 207 - protein, 11 aa, synthetic construct
  SITE 3: note = X is 5-chloro-L-tryptophan
  SITE 4: note = X is 4-(R)-hydroxy-L-proline
  SITE 6: note = X is D-leucine
  SITE 8: note = X is 2-Aminoindane-2-carboxylic acid
  CROSSLNK 7..11: note = cysteine cysteine connected through 2,6-lutidylidene
DWXXEXCXLIC

SEQ ID NO: 208 - protein, 11 aa, synthetic construct
  SITE 1: note = X is L-phosphoserine
  SITE 3: note = X is 5-chloro-L-tryptophan
  SITE 4: note = X is 4-(R)-hydroxy-L-proline
  SITE 6: note = X is D-leucine
  SITE 8: note = X is 4-amino-tetrahydropyran-4-carboxylic acid
  CROSSLNK 7..11: note = cysteine cysteine connected through 2,6-lutidylidene
XWXXEXCXLIC

SEQ ID NO: 209 - protein, 11 aa, synthetic construct
  SITE 3: note = X is 5-chloro-L-tryptophan
  SITE 4: note = X is 4-(R)-hydroxy-L-proline
  SITE 5: note = X is L-phosphoserine
  SITE 6: note = X is D-leucine
  SITE 8: note = X is 4-amino-tetrahydropyran-4-carboxylic acid
  CROSSLNK 7..11: note = cysteine cysteine connected through 2,6-lutidylidene
DWXXXXCXLIC

SEQ ID NO: 210 - protein, 11 aa, synthetic construct
  SITE 2: note = X is 3-chloro-L-phenylalanine
  SITE 3: note = X is 5-chloro-L-tryptophan
  SITE 4: note = X is 4-(R)-hydroxy-L-proline
  SITE 6: note = X is D-leucine
  SITE 8: note = X is 4-amino-tetrahydropyran-4-carboxylic acid
  CROSSLNK 7..11: note = cysteine cysteine connected through 2,6-lutidylidene
DXXXEXCXLIC

SEQ ID NO: 211 - protein, 11 aa, synthetic construct
  SITE 2: note = X is para-chloro-L-phenylalanine
  SITE 3: note = X is 5-chloro-L-tryptophan
  SITE 4: note = X is 4-(R)-hydroxy-L-proline
  SITE 6: note = X is D-leucine
  SITE 8: note = X is 4-amino-tetrahydropyran-4-carboxylic acid
  CROSSLNK 7..11: note = cysteine cysteine connected through 2,6-lutidylidene
DXXXEXCXLIC

SEQ ID NO: 212 - protein, 11 aa, synthetic construct
  SITE 2: note = X is 3,4-dichlor-L-phenylalanine
  SITE 3: note = X is 5-chloro-L-tryptophan
  SITE 4: note = X is 4-(R)-hydroxy-L-proline
  SITE 6: note = X is D-leucine
  SITE 8: note = X is 4-amino-tetrahydropyran-4-carboxylic acid
  CROSSLNK 7..11: note = cysteine cysteine connected through 2,6-lutidylidene
DXXXEXCXLIC

SEQ ID NO: 213 - protein, 11 aa, synthetic construct
  SITE 2: note = X is 2,4-dichloro-L-phenylalanine
  SITE 3: note = X is 5-chloro-L-tryptophan
  SITE 4: note = X is 4-(R)-hydroxy-L-proline
  SITE 6: note = X is D-leucine
  SITE 8: note = X is 4-amino-tetrahydropyran-4-carboxylic acid
  CROSSLNK 7..11: note = cysteine cysteine connected through 2,6-lutidylidene
DXXXEXCXLIC

SEQ ID NO: 214 - protein, 11 aa, synthetic construct
  SITE 3: note = X is 3,4-dichlor-L-phenylalanine
  SITE 4: note = X is 4-(R)-hydroxy-L-proline
  SITE 6: note = X is D-leucine
  SITE 8: note = X is 4-amino-tetrahydropyran-4-carboxylic acid
  CROSSLNK 7..11: note = cysteine cysteine connected through 2,6-lutidylidene
DWXXEXCXLIC

SEQ ID NO: 215 - protein, 10 aa, synthetic construct
  SITE 2: note = X is 5-chloro-L-tryptophan
  SITE 3: note = X is 4-(R)-hydroxy-L-proline
  SITE 5: note = X is D-leucine
  SITE 7: note = X is 4-amino-tetrahydropyran-4-carboxylic acid
  CROSSLNK 6..10: note = cysteine cysteine connected through 2,6-lutidylidene
WXXEXCXLIC

SEQ ID NO: 216 - protein, 11 aa, synthetic construct
  SITE 3: note = X is 5-chloro-L-tryptophan
  SITE 4: note = X is (R)-5,5-dimethyl-1,3-thiazolidine-4-carboxylic acid
  SITE 6: note = X is D-leucine
  SITE 8: note = X is 4-amino-tetrahydropyran-4-carboxylic acid
  CROSSLNK 7..11: note = cysteine cysteine connected through 2,6-lutidylidene
DWXXEXCXLIC

SEQ ID NO: 217 - protein, 11 aa, synthetic construct
  SITE 3: note = X is 5-chloro-L-tryptophan
  SITE 4: note = X is (S)-oxazolidine-4-carboxylic acid
  SITE 6: note = X is D-leucine
  SITE 8: note = X is 4-amino-tetrahydropyran-4-carboxylic acid
  CROSSLNK 7..11: note = cysteine cysteine connected through 2,6-lutidylidene
DWXXEXCXLIC

SEQ ID NO: 218 - protein, 11 aa, synthetic construct
  SITE 3: note = X is 5-chloro-L-tryptophan
  SITE 4: note = X is 4-(R)-hydroxy-L-proline
  SITE 5: note = X is N-methyl-L-glutamic acid
  SITE 8: note = X is 4-amino-tetrahydropyran-4-carboxylic acid
  CROSSLNK 7..11: note = cysteine cysteine connected through 2,6-lutidylidene
DWXXXLCXLIC

SEQ ID NO: 219 - protein, 11 aa, synthetic construct
  SITE 3: note = X is 5-chloro-L-tryptophan
  SITE 4: note = X is 4-(R)-hydroxy-L-proline
  SITE 8: note = X is 4-amino-tetrahydropyran-4-carboxylic acid
  CROSSLNK 7..11: note = cysteine cysteine through 2,6-lutidylidene
DWXXEICXLIC

SEQ ID NO: 220 - protein, 11 aa, synthetic construct
  SITE 3: note = X is 5-chloro-L-tryptophan
  SITE 4: note = X is 4-(R)-hydroxy-L-proline
  SITE 8: note = X is 4-amino-tetrahydropyran-4-carboxylic acid
  CROSSLNK 7..11: note = cysteine cysteine through 2,6-lutidylidene
DWXXEVCXLIC

SEQ ID NO: 221 - protein, 11 aa, synthetic construct
  SITE 2: note = X is 5-hydroxy-L-tryptophane
  SITE 3: note = X is 5-chloro-L-tryptophan
  SITE 4: note = X is 4-(R)-hydroxy-L-proline
  SITE 6: note = X is D-leucine
  SITE 8: note = X is 4-amino-tetrahydropyran-4-carboxylic acid
  CROSSLNK 7..11: note = cysteine cysteine through 2,6-lutidylidene
DXXXEXCXLIC

SEQ ID NO: 222 - protein, 11 aa, synthetic construct
  SITE 2: note = X is 7-fluoro-L-tryptophan
  SITE 3: note = X is 5-chloro-L-tryptophan
  SITE 4: note = X is 4-(R)-hydroxy-L-proline
  SITE 6: note = X is D-leucine
  SITE 8: note = X is 4-amino-tetrahydropyran-4-carboxylic acid
  CROSSLNK 7..11: note = cysteine cysteine through 2,6-lutidylidene
DXXXEXCXLIC

SEQ ID NO: 223 - protein, 11 aa, synthetic construct
  SITE 3: note = X is 5-chloro-L-tryptophan
  SITE 4: note = X is L-thiazolidine-4-carboxylic acid
  SITE 6: note = X is D-leucine
  SITE 8: note = X is 4-amino-tetrahydropyran-4-carboxylic acid
  CROSSLNK 7..11: note = cysteine cysteine through 2,6-lutidylidene
DWXXEXCXLIC

SEQ ID NO: 224 - protein, 11 aa, synthetic construct
  SITE 3: note = X is 5-chloro-L-tryptophan
  SITE 4: note = X is 4-(R)-hydroxy-L-proline
  SITE 5: note = X is D-glutamic acid
  SITE 8: note = X is 4-amino-tetrahydropyran-4-carboxylic acid
  CROSSLNK 7..11: note = cysteine cysteine through 2,6-lutidylidene
DWXXXLCXLIC

SEQ ID NO: 225 - protein, 11 aa, synthetic construct
  SITE 3: note = X is 5-chloro-L-tryptophan
  SITE 4: note = X is 4-(R)-hydroxy-L-proline
  SITE 6: note = X is D-leucine
  SITE 8: note = X is 4-amino-tetrahydropyran-4-carboxylic acid
  CROSSLNK 7..11: note = cysteine cysteine through 2,6-lutidylidene
DFXXEXCXLIC

SEQ ID NO: 226 - protein, 11 aa, synthetic construct
  SITE 2: note = X is 2,3-dichloro-L-phenylalanine
  SITE 3: note = X is 5-chloro-L-tryptophan
  SITE 4: note = X is 4-(R)-hydroxy-L-proline
  SITE 6: note = X is D-leucine
  SITE 8: note = X is 4-amino-tetrahydropyran-4-carboxylic acid
  CROSSLNK 7..11: note = cysteine cysteine through 2,6-lutidylidene
DXXXEXCXLIC

SEQ ID NO: 227 - protein, 11 aa, synthetic construct
  SITE 3: note = X is 7-fluoro-L-tryptophan
  SITE 4: note = X is 4-(R)-hydroxy-L-proline
  SITE 6: note = X is D-leucine
  SITE 8: note = X is 4-amino-tetrahydropyran-4-carboxylic acid
  CROSSLNK 7..11: note = cysteine cysteine through 2,6-lutidylidene
DWXXEXCXLIC

SEQ ID NO: 228 - protein, 11 aa, synthetic construct
  SITE 3: note = X is 5-chloro-L-tryptophan
  SITE 4: note = X is 4-(R)-hydroxy-L-proline
  SITE 6: note = X is D-leucine
  CROSSLNK 7..11: note = cysteine cysteine through 2,6-lutidylidene
DWXXEXCFLIC

SEQ ID NO: 229 - protein, 11 aa, synthetic construct
  SITE 3: note = X is 5-chloro-L-tryptophan
  SITE 4: note = X is 4-(R)-hydroxy-L-proline
  SITE 6: note = X is D-leucine
  SITE 8: note = X is 4-amino-tetrahydropyran-4-carboxylic acid
  CROSSLNK 7..11: note = cysteine cysteine through 2,6-lutidylidene
DWXXEXCXIIC

SEQ ID NO: 230 - protein, 11 aa, synthetic construct
  SITE 3: note = X is 5-chloro-L-tryptophan
  SITE 4: note = X is 4-(R)-hydroxy-L-proline
  SITE 6: note = X is D-leucine
  SITE 8: note = X is 4-amino-tetrahydropyran-4-carboxylic acid
  CROSSLNK 7..11: note = cysteine cysteine through 2,6-lutidylidene
DWXXEXCXRIC

SEQ ID NO: 231 - protein, 11 aa, synthetic construct
  SITE 3: note = X is 5-chloro-L-tryptophan
  SITE 4: note = X is 4-(R)-hydroxy-L-proline
  SITE 6: note = X is D-leucine
  SITE 8: note = X is 4-amino-tetrahydropyran-4-carboxylic acid
  CROSSLNK 7..11: note = cysteine cysteine through 2,6-lutidylidene
DWXXEXCXEIC

SEQ ID NO: 232 - protein, 11 aa, synthetic construct
  SITE 3: note = X is 5-chloro-L-tryptophan
  SITE 4: note = X is 4-(R)-hydroxy-L-proline
  SITE 6: note = X is D-leucine
  SITE 8: note = X is 4-amino-tetrahydropyran-4-carboxylic acid
  CROSSLNK 7..11: note = cysteine cysteine through 2,6-lutidylidene
DWXXEXCXFIC

SEQ ID NO: 233 - protein, 11 aa, synthetic construct
  SITE 3: note = X is 5-chloro-L-tryptophan
  SITE 4: note = X is 4-(R)-hydroxy-L-proline
  SITE 6: note = X is D-leucine
  SITE 8: note = X is 4-amino-tetrahydropyran-4-carboxylic acid
  CROSSLNK 7..11: note = cysteine cysteine through 2,6-lutidylidene
DWXXEXCXWIC

SEQ ID NO: 234 - protein, 11 aa, synthetic construct
  SITE 3: note = X is 5-chloro-L-tryptophan
  SITE 4: note = X is 4-(R)-hydroxy-L-proline
  SITE 6: note = X is D-leucine
  CROSSLNK 7..11: note = cysteine cysteine through 2,6-lutidylidene
DWXXEXCWLIC

SEQ ID NO: 235 - protein, 11 aa, synthetic construct
  SITE 3: note = X is 5-chloro-L-tryptophan
  SITE 4: note = X is 4-(R)-hydroxy-L-proline
  SITE 6: note = X is D-leucine
  CROSSLNK 7..11: note = cysteine cysteine through 2,6-lutidylidene
DWXXEXCELIC

SEQ ID NO: 236 - protein, 11 aa, synthetic construct
  SITE 3: note = X is 5-chloro-L-tryptophan
  SITE 4: note = X is 4-(R)-hydroxy-L-proline
  SITE 6: note = X is D-leucine
  SITE 8: note = X is 4-amino-tetrahydropyran-4-carboxylic acid
  CROSSLNK 7..11: note = cysteine cysteine through 2,6-lutidylidene
DWXXEXCXSIC

SEQ ID NO: 237 - protein, 11 aa, synthetic construct
  SITE 3: note = X is 5-chloro-L-tryptophan
  SITE 4: note = X is 4-(R)-hydroxy-L-proline
  SITE 6: note = X is D-leucine
  SITE 8: note = X is 4-amino-tetrahydropyran-4-carboxylic acid
  CROSSLNK 7..11: note = cysteine cysteine through 2,6-lutidylidene
DWXXEXCXLLC

SEQ ID NO: 238 - protein, 11 aa, synthetic construct
  SITE 3: note = X is 5-chloro-L-tryptophan
  SITE 4: note = X is 4-(R)-hydroxy-L-proline
  SITE 6: note = X is D-leucine
  SITE 8: note = X is 4-amino-tetrahydropyran-4-carboxylic acid
  SITE 10: note = X is (S)-alpha-t-butylglycine
  CROSSLNK 7..11: note = cysteine cysteine through 2,6-lutidylidene
DWXXEXCXLXC

SEQ ID NO: 239 - protein, 11 aa, synthetic construct
  SITE 3: note = X is 5-chloro-L-tryptophan
  SITE 4: note = X is (2S,4R)-4-amino-1-pyrrolidin-2-carboxylic acid
  SITE 6: note = X is D-leucine
  SITE 8: note = X is 4-amino-tetrahydropyran-4-carboxylic acid
  CROSSLNK 7..11: note = cysteine cysteine through 2,6-lutidylidene
DWXXEXCXLIC

SEQ ID NO: 240 - protein, 12 aa, synthetic construct
  CROSSLNK 7..11: note = cysteine cysteine connected through 3-methylbenzylidene
WDWWPELCALIC

SEQ ID NO: 241 - protein, 13 aa, synthetic construct
  CROSSLNK 9..13: note = cysteine cysteine connected through 3-methylbenzylidene
MYDWWPELCQLIC

SEQ ID NO: 242 - protein, 13 aa, synthetic construct
  CROSSLNK 9..13: note = cysteine cysteine connected through 3-methylbenzylidene
MYDWWPELCTLIC

SEQ ID NO: 243 - protein, 13 aa, synthetic construct
  CROSSLNK 9..13: note = cysteine cysteine connected through 3-methylbenzylidene
MYDWWPELCTLIC

SEQ ID NO: 244 - protein, 11 aa, synthetic construct
  CROSSLNK 7..11: note = cysteine cysteine connected through 3-methylbenzylidene
DWWPELCALIC

SEQ ID NO: 245 - protein, 13 aa, synthetic construct
  SITE 7: note = X is D-glutamic acid
  CROSSLNK 9..13: note = cysteine cysteine connected through 3-methylbenzylidene
MYDWWPXLCALIC

SEQ ID NO: 246 - protein, 13 aa, synthetic construct
  SITE 2: note = X is D-tyrosine
  CROSSLNK 9..13: note = cysteine cysteine connected through 3-methylbenzylidene
MXDWWPELCALIC

SEQ ID NO: 247 - protein, 13 aa, synthetic construct
  SITE 8: note = X is D-leucine
  CROSSLNK 9..13: note = cysteine cysteine connected through 3-methylbenzylidene
MYDWWPEXCALIC

SEQ ID NO: 248 - protein, 13 aa, synthetic construct
  SITE 10: note = X is D-alanine
  CROSSLNK 9..13: note = cysteine cysteine connected through 3-methylbenzylidene
MYDWWPELCXLIC

SEQ ID NO: 249 - protein, 13 aa, synthetic construct
  CROSSLNK 9..13: note = cysteine cysteine connected through 3-methylbenzylidene
MADWWPELCALIC

SEQ ID NO: 250 - protein, 11 aa, synthetic construct
  SITE 8: note = X is D-alanine
  SITE 11: note = X is 2-aminoethane-1-thiol
  CROSSLNK 7..11: note = cysteine 2-aminoethane-1-thiol connected through 3-methylbenzylidene
DWWPELCXLIX

SEQ ID NO: 251 - protein, 11 aa, synthetic construct
  SITE 8: note = X is 1-amino-isobutyric acid
  SITE 11: note = X is 2-aminoethane-1-thiol
  CROSSLNK 7..11: note = cysteine 2-aminoethane-1-thiol connected through 3-methylbenzylidene
DWWPELCXLIX

SEQ ID NO: 252 - protein, 11 aa, synthetic construct
  SITE 3: note = X is 3-(1-naphthyl)-L-alanine
  SITE 4: note = X is (2S,3aS,7aS)- octahydroindolecarboxylic acid
  SITE 8: note = X is 1-amino-isobutyric acid
  CROSSLNK 7..11: note = cysteine cysteine connected through 3-methylbenzylidene
DWXXELCXLIC

SEQ ID NO: 253 - protein, 11 aa, synthetic construct
  SITE 3: note = X is 3-(1-naphthyl)-L-alanine
  SITE 4: note = X is (S)-Pipecolic Acid
  SITE 8: note = X is 1-amino-isobutyric acid
  CROSSLNK 7..11: note = cysteine cysteine connected through 3-methylbenzylidene
DWXXELCXLIC

SEQ ID NO: 254 - protein, 13 aa, synthetic construct
  CROSSLNK 7..11: note = cysteine cysteine connected through 3-methylbenzylidene
MYDWWPELCALIC

SEQ ID NO: 255 - protein, 10 aa, synthetic construct
  CROSSLNK 6..10: note = cysteine cysteine connected through 3-methylbenzylidene
WWPELCALIC

SEQ ID NO: 256 - protein, 10 aa, synthetic construct
  SITE 10: note = X is 2-aminoethane-1-thiol
  CROSSLNK 6..10: note = cysteine 2-aminoethane-1-thiol connected through 3-methylbenzylidene
WWPELCALIX

SEQ ID NO: 257 - protein, 10 aa, synthetic construct
  CROSSLNK 6..10: note = cysteine cysteine through 2,6-lutidylidene
WWPELCALIC

SEQ ID NO: 258 - protein, 10 aa, synthetic construct
  SITE 5: note = X is D-leucine
  SITE 7: note = X is 1-amino-isobutyric acid
  CROSSLNK 6..10: note = cysteine cysteine connected through 3-methylbenzylidene
WWPEXCXLIC

SEQ ID NO: 259 - protein, 10 aa, synthetic construct
  SITE 7: note = X is 1-amino-isobutyric acid
  CROSSLNK 6..10: note = cysteine cysteine connected through 3-methylbenzylidene
WWPELCXLIC

SEQ ID NO: 260 - protein, 10 aa, synthetic construct
  SITE 7: note = X is 1-amino-isobutyric acid
  SITE 8: note = X is L-homoleucine
  CROSSLNK 6..10: note = cysteine cysteine connected through 3-methylbenzylidene
WWPELCXXIC

SEQ ID NO: 261 - protein, 10 aa, synthetic construct
  SITE 7: note = X is 4-amino-tetrahydropyran-4-carboxylic acid
  CROSSLNK 6..10: note = cysteine cysteine connected through 3-methylbenzylidene
WWPELCXLIC

SEQ ID NO: 262 - protein, 11 aa, synthetic construct
  SITE 2: note = X is ortho-chloro-L-phenylalanine
  SITE 3: note = X is 5-chloro-L-tryptophan
  SITE 4: note = X is 4-(R)-hydroxy-L-proline
  SITE 6: note = X is D-leucine
  SITE 8: note = X is 4-amino-tetrahydropyran-4-carboxylic acid
  CROSSLNK 7..11: note = cysteine cysteine through 2,6-lutidylidene
DXXXEXCXLIC

SEQ ID NO: 263 - protein, 11 aa, synthetic construct
  SITE 2: note = X is 3,5-dichloro-L-phenylalanine
  SITE 3: note = X is 5-chloro-L-tryptophan
  SITE 4: note = X is 4-(R)-hydroxy-L-proline
  SITE 6: note = X is D-leucine
  SITE 8: note = X is 4-amino-tetrahydropyran-4-carboxylic acid
  CROSSLNK 7..11: note = cysteine cysteine through 2,6-lutidylidene
DXXXEXCXLIC

SEQ ID NO: 264 - protein, 11 aa, synthetic construct
  SITE 3: note = X is 2,3-dichloro-L-phenylalanine
  SITE 4: note = 4-(R)-hydroxy-L-proline
  SITE 6: note = X is D-leucine
  SITE 8: note = X is 4-amino-tetrahydropyran-4-carboxylic acid
  CROSSLNK 7..11: note = cysteine cysteine through 2,6-lutidylidene
DWXXEXCXLIC

SEQ ID NO: 265 - protein, 11 aa, synthetic construct
  SITE 3: note = X is 5-chloro-L-tryptophan
  SITE 4: note = X is 4-(R)-hydroxy-L-proline
  SITE 6: note = X is D-leucine
  SITE 8: note = X is 4-amino-tetrahydropyran-4-carboxylic acid
  CROSSLNK 7..11: note = cysteine cysteine through 2,6-lutidylidene
DWXXEXCXLFC

SEQ ID NO: 266 - protein, 11 aa, synthetic construct
  SITE 3: note = X is 7-aza-L-tryptophan
  SITE 4: note = X is 4-(R)-hydroxy-L-proline
  SITE 6: note = X is D-leucine
  SITE 8: note = X is 4-amino-tetrahydropyran-4-carboxylic acid
  CROSSLNK 7..11: note = cysteine cysteine through 2,6-lutidylidene
DWXXEXCXLIC

SEQ ID NO: 267 - protein, 11 aa, synthetic construct
  SITE 5: note = X is D-glutamic acid
  SITE 8: note = X is cysteine cysteine through 2,6-lutidylidene
  CROSSLNK 7..11: note = cysteine cysteine connected through 3-methylbenzylidene
DWWPXLCXLIC

SEQ ID NO: 268 - protein, 11 aa, synthetic construct
  SITE 1: note = X is D-aspartic acid
  SITE 8: note = X is D-alanine
  CROSSLNK 7..11: note = cysteine cysteine connected through 3-methylbenzylidene
XWWPELCXLIC

SEQ ID NO: 269 - protein, 11 aa, synthetic construct
  SITE 2: note = X is 3-(3-benzothienyl)-L-alanine
  SITE 8: note = X is 1-amino-isobutyric acid
  CROSSLNK 7..11: note = cysteine cysteine connected through 3-methylbenzylidene
DXWPELCXLIC

SEQ ID NO: 270 - protein, 11 aa, synthetic construct
  SITE 5: note = X is N-methyl-L-glutamic acid
  SITE 8: note = X is 1-amino-isobutyric acid
  CROSSLNK 7..11: note = cysteine cysteine connected through 3-methylbenzylidene
DWWPXLCXLIC

SEQ ID NO: 271 - protein, 11 aa, synthetic construct
  SITE 8: note = X is 1-amino-isobutyric acid
  SITE 9: note = X is (S)-alpha-t-butylglycine
  CROSSLNK 7..11: note = cysteine cysteine connected through 3-methylbenzylidene
DWWPELCXXIC

SEQ ID NO: 272 - protein, 11 aa, synthetic construct
  SITE 2: note = X is 5-chloro-L-tryptophan
  SITE 8: note = X is 1-amino-isobutyric acid
  CROSSLNK 7..11: note = cysteine cysteine connected through 3-methylbenzylidene
DXWPELCXLIC

SEQ ID NO: 273 - protein, 11 aa, synthetic construct
  SITE 6: note = X is D-leucine
  SITE 8: note = X is 1-amino-isobutyric acid
  CROSSLNK 7..11: note = cysteine cysteine connected through 3-methylbenzylidene
DWWPEXCXLIC

SEQ ID NO: 274 - protein, 11 aa, synthetic construct
  SITE 6: note = X is D-leucine
  SITE 8: note = X is 1-amino-isobutyric acid
  CROSSLNK 7..11: note = cysteine cysteine connected through 3-methylbenzylidene
DWWPEXCXLIC

SEQ ID NO: 275 - protein, 11 aa, synthetic construct
  SITE 3: note = X is 6-chloro-L-tryptophan
  SITE 6: note = X is D-leucine
  SITE 8: note = X is 1-amino-isobutyric acid
  CROSSLNK 7..11: note = cysteine cysteine connected through 3-methylbenzylidene
DWXPEXCXLIC

SEQ ID NO: 276 - protein, 11 aa, synthetic construct
  SITE 2: note = X is 6-fluoro-L-tryptophan
  SITE 6: note = X is D-leucine
  SITE 8: note = X is 1-amino-isobutyric acid
  SITE 11: note = X is 2-aminoethane-1-thiol
  CROSSLNK 7..11: note = cysteine 2-aminoethane-1-thiol connected through 3-methylbenzylidene
DXWPEXCXLIX

SEQ ID NO: 277 - protein, 11 aa, synthetic construct
  SITE 3: note = X is 3-(1-naphthyl)-L-alanine
  SITE 6: note = X is L-homoleucine
  SITE 8: note = X is 1-amino-isobutyric acid
  CROSSLNK 7..11: note = cysteine cysteine connected through 3-methylbenzylidene
DWXPEXCXLIC

SEQ ID NO: 278 - protein, 10 aa, synthetic construct
  SITE 3: note = X is 4-(R)-hydroxy-L-proline
  SITE 8: note = X is 1-amino-isobutyric acid
  CROSSLNK 6..10: note = cysteine cysteine connected through 3-methylbenzylidene
WWXELCXLIC

SEQ ID NO: 279 - protein, 11 aa, synthetic construct
  SITE 3: note = X is 5-chloro-L-tryptophan
  SITE 6: note = X is D-leucine
  SITE 8: note = X is 1-amino-isobutyric acid
  SITE 7: note = X is (S)-2,3-Diaminopropionic acid
  SITE 11: note = X is 2-aminoethane-1-thiol
  CROSSLNK 7..11: note = (S)-2,3-Diaminopropionic acid 2-aminoethane-1-thiol connected though 3-methylbenzoyl
DWXPEXXXLIX

SEQ ID NO: 280 - protein, 11 aa, synthetic construct
  SITE 3: note = X is 5-chloro-L-tryptophan
  SITE 6: note = X is D-leucine
  SITE 8: note = X is 1-amino-isobutyric acid
  SITE 11: note = ethylendiamine
  CROSSLNK 7..11: note = cysteine ethylendiamine connected through 3-carboxybenzyl
DWXPEXCXLIX

SEQ ID NO: 281 - protein, 11 aa, synthetic construct
  SITE 3: note = X is 5-chloro-L-tryptophan
  SITE 4: note = X is 4-(R)-hydroxy-L-proline
  SITE 6: note = X is D-leucine
  SITE 8: note = X is 4-amino-tetrahydropyran-4-carboxylic acid
  CROSSLNK 7..11: note = cysteine cysteine connected through maleimide
DWXXEXCXLIC

SEQ ID NO: 282 - protein, 11 aa, synthetic construct
  SITE 3: note = X is 5-chloro-L-tryptophan
  SITE 4: note = X is 4-(R)-hydroxy-L-proline
  SITE 6: note = X is D-leucine
  SITE 8: note = X is 4-amino-tetrahydropyran-4-carboxylic acid
  SITE 11: note = X is L-homocysteine
  CROSSLNK 7..11: note = cysteine L-homocysteine connected through maleimide
DWXXEXCXLIX

SEQ ID NO: 283 - protein, 11 aa, synthetic construct
  SITE 3: note = X is 5-chloro-L-tryptophan
  SITE 4: note = X is 4-(R)-hydroxy-L-proline
  MOD_RES 6: note = X is D-leucine
  SITE 7: note = X is L-homocysteine
  SITE 8: note = X is 4-amino-tetrahydropyran-4-carboxylic acid
  CROSSLNK 7..11: note = L-homocysteine cysteine connected through maleimide
DWXXEXXXLIC

SEQ ID NO: 284 - protein, 11 aa, synthetic construct
  SITE 3: note = X is 5-chloro-L-tryptophan
  SITE 4: note = X is 4-(R)-hydroxy-L-proline
  SITE 6: note = X is D-leucine
  SITE 7: note = X is L-homocysteine
  SITE 8: note = X is 4-amino-tetrahydropyran-4-carboxylic acid
  SITE 11: note = X is L-homocysteine
  CROSSLNK 7..11: note = connceted through S-S
DWXXEXXXLIX

SEQ ID NO: 285 - protein, 11 aa, synthetic construct
  SITE 3: note = X is 5-chloro-L-tryptophan
  SITE 4: note = X is (2S,4R)-4-amino-1-pyrrolidin-2-carboxylic acid
  SITE 6: note = X is D-leucine
  SITE 8: note = X is 4-aminopiperidine-4-carboxylic acid
  CROSSLNK 7..11: note = cysteine cysteine through 2,6-lutidylidene
DWXXEXCXLIC

SEQ ID NO: 286 - protein, 11 aa, synthetic construct
  SITE 3: note = X is 5-chloro-L-tryptophan
  SITE 4: note = X is (2S,4R)-4-amino-1-pyrrolidin-2-carboxylic acid
  SITE 6: note = X is D-leucine
  SITE 8: note = X is alpha-methyl lysine
  CROSSLNK 7..11: note = cysteine cysteine through 2,6-lutidylidene
DWXXEXCXLIC

SEQ ID NO: 287 - protein, 11 aa, synthetic construct
  SITE 3: note = X is 5-chloro-L-tryptophan
  SITE 4: note = X is (2S,4R)-4-amino-1-pyrrolidin-2-carboxylic acid
  SITE 6: note = X is D-leucine
  SITE 8: note = X is 4-aminopiperidine-4-carboxylic acid
  CROSSLNK 7..11: note = cysteine cysteine through 2,6-lutidylidene
DWXXEXCXLIC

SEQ ID NO: 288 - protein, 11 aa, synthetic construct
  SITE 3: note = X is 5-chloro-L-tryptophan
  SITE 4: note = X is (2S,4R)-4-amino-1-pyrrolidin-2-carboxylic acid
  SITE 6: note = X is D-leucine
  SITE 8: note = X is 4-aminopiperidine-4-carboxylic acid
  CROSSLNK 7..11: note = cysteine cysteine through 2,6-lutidylidene
DWXXEXCXLIC

SEQ ID NO: 289 - protein, 11 aa, synthetic construct
  SITE 3: note = X is 5-chloro-L-tryptophan
  SITE 4: note = X is (2S,4R)-4-amino-1-pyrrolidin-2-carboxylic acid
  SITE 6: note = X is D-leucine
  SITE 8: note = X is 4-aminopiperidine-4-carboxylic acid
  CROSSLNK 7..11: note = cysteine cysteine through 2,6-lutidylidene
DWXXEXCXLIC

SEQ ID NO: 290 - protein, 11 aa, synthetic construct
  SITE 3: note = X is 5-chloro-L-tryptophan
  SITE 4: note = X is 4-(R)-hydroxy-L-proline
  SITE 6: note = X is D-leucine
  SITE 8: note = X is 4-amino-tetrahydropyran-4-carboxylic acid
  CROSSLNK 7..11: note = cysteine cysteine through 2,6-lutidylidene
DWXXKXCXLIC

SEQ ID NO: 291 - protein, 11 aa, synthetic construct
  SITE 3: note = X is 5-chloro-L-tryptophan
  SITE 4: note = X is 4-(R)-hydroxy-L-proline
  SITE 6: note = X is D-leucine
  SITE 8: note = X is 4-amino-tetrahydropyran-4-carboxylic acid
  CROSSLNK 7..11: note = cysteine cysteine through 2,6-lutidylidene
DWXXRXCXLIC

SEQ ID NO: 292 - protein, 11 aa, synthetic construct
  SITE 3: note = X is 5-chloro-L-tryptophan
  SITE 4: note = X is 4-(R)-hydroxy-L-proline
  SITE 6: note = X is D-leucine
  SITE 8: note = X is 4-amino-tetrahydropyran-4-carboxylic acid
  CROSSLNK 7..11: note = cysteine cysteine through 2,6-lutidylidene
DWXXHXCXLIC

SEQ ID NO: 293 - protein, 11 aa, synthetic construct
  SITE 3: note = X is 5-chloro-L-tryptophan
  SITE 4: note = X is 4-(R)-hydroxy-L-proline
  SITE 5: note = X is homolysine
  SITE 6: note = X is D-leucine
  SITE 8: note = X is 4-amino-tetrahydropyran-4-carboxylic acid
  CROSSLNK 7..11: note = cysteine cysteine through 2,6-lutidylidene
DWXXXXCXLIC

SEQ ID NO: 294 - protein, 11 aa, synthetic construct
  SITE 3: note = X is 5-chloro-L-tryptophan
  SITE 2: note = X is 5-chloro-L-tryptophan
  SITE 4: note = X is 4-(R)-hydroxy-L-proline
  SITE 6: note = X is D-leucine
  SITE 8: note = X is 4-amino-tetrahydropyran-4-carboxylic acid
  CROSSLNK 7..11: note = cysteine cysteine through 2,6-lutidylidene
DXXXEXCXLIC

SEQ ID NO: 295 - protein, 11 aa, synthetic construct
  SITE 2: note = X is 3-(1-naphthyl)-L-alanine
  SITE 3: note = X is 5-chloro-L-tryptophan
  SITE 4: note = X is 4-(R)-hydroxy-L-proline
  SITE 6: note = is D-leucine
  SITE 8: note = X is 4-amino-tetrahydropyran-4-carboxylic acid
  CROSSLNK 7..11: note = cysteine cysteine through 2,6-lutidylidene
DXXXEXCXLIC

SEQ ID NO: 296 - protein, 11 aa, synthetic construct
  SITE 1: note = X is D-aspartic acid
  SITE 3: note = X is 5-chloro-L-tryptophan
  SITE 4: note = X is 4-(R)-hydroxy-L-proline
  SITE 6: note = X is D-leucine
  SITE 8: note = X is 4-amino-tetrahydropyran-4-carboxylic acid
  CROSSLNK 7..11: note = cysteine cysteine through 2,6-lutidylidene
XWXXEXCXLIC

SEQ ID NO: 297 - protein, 11 aa, synthetic construct
  SITE 2: note = X is 5-fluoro-L-tryptophan
  SITE 3: note = X is 5-chloro-L-tryptophan
  SITE 6: note = X is D-leucine
  SITE 4: note = X is 4-(R)-hydroxy-L-proline
  SITE 8: note = X is 4-amino-tetrahydropyran-4-carboxylic acid
  CROSSLNK 7..11: note = cysteine cysteine through 2,6-lutidylidene
DXXXEXCXLIC

SEQ ID NO: 298 - protein, 11 aa, synthetic construct
  SITE 3: note = X is 5-chloro-L-tryptophan
  SITE 4: note = X is 4-(R)-hydroxy-L-proline
  SITE 6: note = X is D-leucine
  SITE 8: note = X is 4-amino-tetrahydropyran-4-carboxylic acid
  SITE 9: note = X is L-cyclo-hexyl-alanine
  CROSSLNK 7..11: note = cysteine cysteine through 2,6-lutidylidene
DWXXEXCXXIC

SEQ ID NO: 299 - protein, 11 aa, synthetic construct
  SITE 3: note = X is 5-chloro-L-tryptophan
  SITE 4: note = X is 4-(R)-hydroxy-L-proline
  SITE 6: note = X is D-leucine
  SITE 8: note = X is 4-amino-tetrahydropyran-4-carboxylic acid
  CROSSLNK 7..11: note = cysteine cysteine through 2,6-lutidylidene
DWXXSXCXLIC

SEQ ID NO: 300 - protein, 11 aa, synthetic construct
  SITE 3: note = X is 5-chloro-L-tryptophan
  SITE 4: note = X is 4-(R)-hydroxy-L-proline
  SITE 6: note = X is D-leucine
  SITE 8: note = X is 4-amino-tetrahydropyran-4-carboxylic acid
  CROSSLNK 7..11: note = cysteine cysteine through 2,6-lutidylidene
DWXXVXCXLIC

SEQ ID NO: 301 - protein, 11 aa, synthetic construct
  SITE 3: note = X is 5-chloro-L-tryptophan
  SITE 4: note = X is 4-(R)-hydroxy-L-proline
  SITE 6: note = X isD-leucine
  SITE 8: note = X is 4-amino-tetrahydropyran-4-carboxylic acid
  CROSSLNK 7..11: note = cysteine cysteine through 2,6-lutidylidene
DWXXAXCXLIC

SEQ ID NO: 302 - protein, 11 aa, synthetic construct
  SITE 2: note = X is ortho-chloro-L-phenylalanine
  SITE 3: note = X is 5-chloro-L-tryptophan
  SITE 4: note = X is 4-(R)-hydroxy-L-proline
  SITE 6: note = X is D-leucine
  SITE 8: note = X is 4-amino-tetrahydropyran-4-carboxylic acid
  CROSSLNK 7..11: note = cysteine cysteine through 2,6-lutidylidene
DXXXEXCXLIC

SEQ ID NO: 303 - protein, 11 aa, synthetic construct
  SITE 3: note = X is 5-chloro-L-tryptophan
  SITE 4: note = X is 4-(R)-hydroxy-L-proline
  SITE 6: note = X is D-leucine
  SITE 8: note = X is 4-amino-tetrahydropyran-4-carboxylic acid
  CROSSLNK 7..11: note = cysteine cysteine through 2,6-lutidylidene
DWXXEXCXLFC

SEQ ID NO: 304 - protein, 11 aa, synthetic construct
  SITE 3: note = X is 5-chloro-L-tryptophan
  SITE 4: note = X is 4-(R)-hydroxy-L-proline
  SITE 6: note = X is D-leucine
  SITE 8: note = X is 4-amino-tetrahydropyran-4-carboxylic acid
  CROSSLNK 7..11: note = cysteine cysteine through 2,6-lutidylidene
DWXXEXCXLFC

SEQ ID NO: 305 - protein, 11 aa, synthetic construct
  SITE 2: note = X is ortho-chloro-L-phenylalanine
  SITE 3: note = X is 5-chloro-L-tryptophan
  SITE 4: note = X is (2S,4R)-4-amino-1-pyrrolidin-2-carboxylic acid
  SITE 6: note = X is D-leucine
  SITE 8: note = X is 4-amino-tetrahydropyran-4-carboxylic acid
  CROSSLNK 7..11: note = cysteine cysteine through 2,6-lutidylidene
DXXXEXCXLIC

SEQ ID NO: 306 - protein, 11 aa, synthetic construct
  SITE 3: note = X is 5-chloro-L-tryptophan
  SITE 4: note = X is (2S,4R)-4-amino-1-pyrrolidin-2-carboxylic acid
  SITE 6: note = X is D-leucine
  SITE 8: note = X is 4-amino-tetrahydropyran-4-carboxylic acid
  CROSSLNK 7..11: note = cysteine cysteine through 2,6-lutidylidene
DWXXEXCXLFC

SEQ ID NO: 307 - protein, 11 aa, synthetic construct
  SITE 2: note = X is ortho-chloro-L-phenylalanine
  SITE 3: note = X is 5-chloro-L-tryptophan
  SITE 4: note = X is 4-(R)-hydroxy-L-proline
  SITE 6: note = X is D-leucine
  SITE 8: note = X is 4-aminopiperidine-4-carboxylic acid
  CROSSLNK 7..11: note = cysteine cysteine through 2,6-lutidylidene
DXXXEXCXLIC

SEQ ID NO: 308 - protein, 11 aa, synthetic construct
  SITE 3: note = X is 5-chloro-L-tryptophan
  SITE 4: note = X is 4-(R)-hydroxy-L-proline
  SITE 6: note = X is D-leucine
  SITE 8: note = X is 4-aminopiperidine-4-carboxylic acid
  CROSSLNK 7..11: note = cysteine cysteine through 2,6-lutidylidene
DWXXEXCXLFY

SEQ ID NO: 309 - protein, 11 aa, synthetic construct
  SITE 3: note = X is 3-(1-naphthyl)-L-alanine
  SITE 4: note = X is (2S,4R)-4-amino-1-pyrrolidin-2-carboxylic acid
  SITE 6: note = X is L-homoleucine
  SITE 8: note = X is 1-amino-isobutyric acid
  CROSSLNK 7..11: note = cysteine cysteine connected through 3-methylbenzylidene
DWXXEXCXLIC

SEQ ID NO: 310 - protein, 11 aa, synthetic construct
  SITE 3: note = X is 5-chloro-L-tryptophan
  SITE 4: note = X is 4-(R)-hydroxy-L-proline
  SITE 5: note = X is L-homoarginine
  SITE 6: note = X is D-leucine
  SITE 8: note = X is 4-amino-tetrahydropyran-4-carboxylic acid
  CROSSLNK 7..11: note = cysteine cysteine through 2,6-lutidylidene
DWXXXXCXLIC

SEQ ID NO: 311 - protein, 11 aa, synthetic construct
  SITE 3: note = X is 5-chloro-L-tryptophan
  SITE 4: note = X is 4-(R)-hydroxy-L-proline
  SITE 6: note = X is D-leucine
  SITE 8: note = X is 4-amino-tetrahydropyran-4-carboxylic acid
  CROSSLNK 7..11: note = cysteine cysteine through 2,6-lutidylidene
DWXXFXCXLIC

SEQ ID NO: 312 - protein, 11 aa, synthetic construct
  SITE 3: note = X is 5-chloro-L-tryptophan
  SITE 4: note = X is 4-(R)-hydroxy-L-proline
  SITE 6: note = X is D-leucine
  SITE 8: note = X is 4-amino-tetrahydropyran-4-carboxylic acid
  CROSSLNK 7..11: note = cysteine cysteine through 2,6-lutidylidene
DWXXWXCXLIC

SEQ ID NO: 313 - protein, 11 aa, synthetic construct
  SITE 3: note = X is 5-chloro-L-tryptophan
  SITE 4: note = X is 4-(R)-hydroxy-L-proline
  SITE 6: note = X is D-leucine
  SITE 8: note = X is 4-amino-tetrahydropyran-4-carboxylic acid
  CROSSLNK 7..11: note = cysteine cysteine through 2,6-lutidylidene
DWXXPXCXLIC

SEQ ID NO: 314 - protein, 11 aa, synthetic construct
  SITE 3: note = X is 5-chloro-L-tryptophan
  SITE 2: note = X is ortho-chloro-L-phenylalanine
  SITE 4: note = X is 4-(R)-hydroxy-L-proline
  SITE 6: note = X is D-leucine
  SITE 8: note = X is 4-amino-tetrahydropyran-4-carboxylic acid
  CROSSLNK 7..11: note = cysteine cysteine through 2,6-lutidylidene
DXXXEXCXLIC

SEQ ID NO: 315 - protein, 11 aa, synthetic construct
  SITE 3: note = X is 3-(1-naphthyl)-L-alanine
  SITE 6: note = X is L-homoleucine
  SITE 8: note = X is 4-aminopiperidine-4-carboxylic acid
  CROSSLNK 7..11: note = cysteine cysteine connected through 3-methylbenzylidene
DWXPEXCXLIC

SEQ ID NO: 316 - protein, 11 aa, synthetic construct
  SITE 3: note = X is 3,5-dichloro-L-phenylalanine
  SITE 4: note = X is 4-(R)-hydroxy-L-proline
  SITE 6: note = X is D-leucine
  SITE 8: note = 4-amino-tetrahydropyran-4-carboxylic acid
  CROSSLNK 7..11: note = cysteine cysteine through 2,6-lutidylidene
DWXXEXCXLIC

SEQ ID NO: 317 - protein, 11 aa, synthetic construct
  SITE 6: note = X is D-leucine
  SITE 8: note = X is D-alanine
  CROSSLNK 7..11: note = cysteine cysteine connected through 3-methylbenzylidene
DWWPEXCXLIC

SEQ ID NO: 318 - protein, 11 aa, synthetic construct
  SITE 5: note = X is D-glutamic acid
  SITE 8: note = X is D alanine
  CROSSLNK 7..11: note = cysteine cysteine connected through 3-methylbenzylidene
DWWPXLCXLIC

SEQ ID NO: 319 - protein, 11 aa, synthetic construct
  SITE 1: note = D-aspartic acid
  SITE 8: note = X is D-alanine
  CROSSLNK 7..11: note = cysteine cysteine connected through 3-methylbenzylidene
XWWPELCXLIC

SEQ ID NO: 320 - protein, 11 aa, synthetic construct
  SITE 8: note = X is 1-amino-isobutyric acid
  SITE 10: note = X is L-cyclo-hexyl-alanine
  CROSSLNK 7..11: note = cysteine cysteine connected through 3-methylbenzylidene
DWWPELCXLXC

SEQ ID NO: 321 - protein, 11 aa, synthetic construct
  SITE 6: note = is D-leucine
  SITE 8: note = X is 1-amino-isobutyric acid
  CROSSLNK 7..11: note = cysteine cysteine connected through 3-methylbenzylidene
DWWPEXCXLIC

SEQ ID NO: 322 - protein, 11 aa, synthetic construct
  SITE 6: note = X is D-leucine
  SITE 8: note = X is d-alanine
  CROSSLNK 7..11: note = cysteine cysteine connected through 3-methylbenzylidene
DWWPEXCXLIC

SEQ ID NO: 323 - protein, 10 aa, synthetic construct
  SITE 5: note = X is D-leucine
  SITE 7: note = is D-alanine
WWPEXCXLIC

SEQ ID NO: 324 - protein, 10 aa, synthetic construct
  SITE 5: note = X is D-leucine
  SITE 7: note = X is D-alanine
WWPEXCXLIC

SEQ ID NO: 325 - protein, 10 aa, synthetic construct
  SITE 5: note = X is D-leucine
  SITE 7: note = X is D-alanine
WWPECXCLIC

SEQ ID NO: 326 - protein, 11 aa, synthetic construct
  SITE 3: note = X is N-methyl-L-tryptophan
  SITE 8: note = X is 1-amino-isobutyric acid
DWXPELCXLIC

SEQ ID NO: 327 - protein, 11 aa, synthetic construct
  SITE 6: note = X is N-methyl-L-leucine
  SITE 8: note = X is 1-amino-isobutyric acid
DWWPEXCXLIC

SEQ ID NO: 328 - protein, 11 aa, synthetic construct
  SITE 7: note = X is N-methyl-L-cysteine
  SITE 8: note = 1-amino-isobutyric acid
DWWPELXXLIC

SEQ ID NO: 329 - protein, 11 aa, synthetic construct
  SITE 8: note = X is 1-amino-isobutyric acid
  SITE 11: note = X is N-methyl-L-cysteine
DWWPELCXLIX

SEQ ID NO: 330 - protein, 11 aa, synthetic construct
  SITE 2: note = X is 3-(1-naphthyl)-L-alanine
  SITE 8: note = X is 1-amino-isobutyric acid
DXWPELCXLIC

SEQ ID NO: 331 - protein, 11 aa, synthetic construct
  SITE 3: note = X is 3-(2-naphthyl)-L-alanine
  SITE 8: note = X is 1-amino-isobutyric acid
DWXPELCXLIC

SEQ ID NO: 332 - protein, 11 aa, synthetic construct
  SITE 3: note = X is 3-(2-naphthyl)-L-alanine
  SITE 8: note = X is 1-amino-isobutyric acid
DWXPELCXLIC

SEQ ID NO: 333 - protein, 11 aa, synthetic construct
  SITE 8: note = X is 1-amino-isobutyric acid
  SITE 6: note = X is alpha-methyl-L-leucine
DWWPEXCXLIC

SEQ ID NO: 334 - protein, 11 aa, synthetic construct
  SITE 3: note = X is N-methyl-L-tryptophan
  SITE 8: note = X is 1-amino-isobutyric acid
DXWPELCXLIC

SEQ ID NO: 335 - protein, 9 aa, synthetic construct
  SITE 1: note = X is 3-(1-naphthyl)-L-alanine
  SITE 6: note = X is 1-amino-isobutyric acid
XPELCXLIC

SEQ ID NO: 336 - protein, 8 aa, synthetic construct
  SITE 5: note = X is 1-amino-isobutyric acid
PELCXLIC

SEQ ID NO: 337 - protein, 11 aa, synthetic construct
  SITE 3: note = X is 3-(1-naphthyl)-L-alanine
  SITE 6: note = X is D-leucine
  SITE 10: note = X is D-valine
DWXPEXCALXC

SEQ ID NO: 338 - protein, 10 aa, synthetic construct
  SITE 3: note = X is 3-(1-naphthyl)-L-alanine
  SITE 6: note = X is D-leucine
  SITE 8: note = X is 1-amino-isobutyric acid
WXPELCXLIC

SEQ ID NO: 339 - protein, 11 aa, synthetic construct
  SITE 3: note = X is 5-hydroxy-L-tryptophane
  SITE 6: note = X is D-leucine
  SITE 8: note = X is 1-amino-isobutyric acid
DWXPEXCXLIC

SEQ ID NO: 340 - protein, 11 aa, synthetic construct
  SITE 2: note = X is 7-aza-L-tryptophan
  SITE 3: note = X is 3-(1-naphthyl)-L-alanine
  SITE 6: note = X is D-leucine
  SITE 8: note = X is 1-amino-isobutyric acid
DXXPEXCXLIC

SEQ ID NO: 341 - protein, 11 aa, synthetic construct
  SITE 4: note = X is 5-chloro-L-tryptophan
  SITE 5: note = X is D-leucine
  SITE 10: note = X is 1-amino-isobutyric acid
  SITE 11: note = X is 2-aminoethane-1-thiol
WDPXXECLIXX

SEQ ID NO: 342 - protein, 11 aa, synthetic construct
  SITE 1: note = X isD-leucine
  SITE 4: note = X is 5-chloro-L-tryptophan
  SITE 9: note = X is 1-amino-isobutyric acid
  SITE 11: note = X is 2-aminoethane-1-thiol
XDPXWLCEXIX

SEQ ID NO: 343 - protein, 11 aa, synthetic construct
  SITE 1: note = X is 5-chloro-L-tryptophan
  SITE 4: note = X is D-leucine
  SITE 10: note = X is 1-amino-isobutyric acid
  SITE 11: note = X is 2-aminoethane-1-thiol
XPEXDWCILXX

SEQ ID NO: 344 - protein, 13 aa, synthetic construct
  SITE 3: note = X is D-aspartic acid
MYXWWPELCALIC

SEQ ID NO: 345 - protein, 13 aa, synthetic construct
  SITE 4: note = X is D-tryptophan
MYDXWPELCALIC

SEQ ID NO: 346 - protein, 13 aa, synthetic construct
  SITE 9: note = X is D-cysteine
MYDWWPELXALIC

SEQ ID NO: 347 - protein, 13 aa, synthetic construct
MYDWWPELCALIC

SEQ ID NO: 348 - protein, 13 aa, synthetic construct
MYDWWAELCALIC

SEQ ID NO: 349 - protein, 13 aa, synthetic construct
MYDWWPELCAAIC

SEQ ID NO: 350 - protein, 13 aa, synthetic construct
MYDWWPGLCALIC

SEQ ID NO: 351 - protein, 10 aa, synthetic construct
  SITE 7: note = X is D-alanine
WWPELCXLIC

SEQ ID NO: 352 - protein, 11 aa, synthetic construct
  SITE 3: note = X is 3-(1-naphthyl)-L-alanine
  SITE 6: note = X is L-homoleucine
  SITE 8: note = X is 1-amino-isobutyric acid
DWXPEXCXLIC

SEQ ID NO: 353 - protein, 11 aa, synthetic construct
  SITE 1: note = X is L-cysteic acid
  SITE 3: note = X is 5-chloro-L-tryptophan
  SITE 4: note = X is 4-(R)-hydroxy-L-proline
  SITE 6: note = X is D-leucine
  SITE 8: note = X is 1-amino-isobutyric acid
  SITE 11: note = X is 2-aminoethane-1-thiol
XGXXEXCXLIX

SEQ ID NO: 354 - protein, 11 aa, synthetic construct
XWWPELCALIC

SEQ ID NO: 355 - protein, 10 aa, synthetic construct
  SITE 5: note = X is D-leucine
  SITE 7: note = X is D-alanine
WWPEXCXLIC

SEQ ID NO: 356 - protein, 11 aa, synthetic construct
  SITE 3: note = X is 3-chloro-L-phenylalanine
  SITE 4: note = X is 4-(R)-hydroxy-L-proline
  SITE 6: note = X is D-leucine
  SITE 8: note = X is 4-amino-tetrahydropyran-4-carboxylic acid
DWXXEXCXLIC

SEQ ID NO: 357 - protein, 11 aa, synthetic construct
  SITE 3: note = X is 5-chloro-L-tryptophan
  SITE 4: note = X is 4-(R)-hydroxy-L-proline
  SITE 6: note = X is D-leucine
  SITE 8: note = X is 4-amino-tetrahydropyran-4-carboxylic acid
DWXXEXCXLSC

SEQ ID NO: 358 - protein, 11 aa, synthetic construct
  SITE 2: note = X is 3,4-dichlor-L-phenylalanine
  SITE 3: note = X is 3,4-dichlor-L-phenylalanine
  SITE 4: note = X is 4-(R)-hydroxy-L-proline
  SITE 6: note = X is D-leucine
  SITE 8: note = X is 4-amino-tetrahydropyran-4-carboxylic acid
DXXXEXCXLIC

SEQ ID NO: 359 - protein, 10 aa, synthetic construct
WWPELCALIC

SEQ ID NO: 360 - protein, 9 aa, synthetic construct
WPELCALIC

SEQ ID NO: 361 - protein, 8 aa, synthetic construct
PELCALIC

SEQ ID NO: 362 - protein, 7 aa, synthetic construct
ELCALIC

SEQ ID NO: 363 - protein, 13 aa, synthetic construct
  SITE 6: note = X is D-proline
MYDWWXELCALIC

SEQ ID NO: 364 - protein, 13 aa, synthetic construct
  SITE 5: note = X is D-tryptophan
MYDWXPELCALIC

SEQ ID NO: 365 - protein, 13 aa, synthetic construct
  SITE 11: note = X is D-leucine
MYDWWPELCAXIC

SEQ ID NO: 366 - protein, 13 aa, synthetic construct
MYDWWPELCALIC

SEQ ID NO: 367 - protein, 13 aa, synthetic construct
MYAWWPELCALIC

SEQ ID NO: 368 - protein, 13 aa, synthetic construct
MYDAWPELCALIC

SEQ ID NO: 369 - protein, 13 aa, synthetic construct
MYDWAPELCALIC

SEQ ID NO: 370 - protein, 13 aa, synthetic construct
MYDWWPALCALIC

SEQ ID NO: 371 - protein, 13 aa, synthetic construct
MYDWWPEACALIC

SEQ ID NO: 372 - protein, 13 aa, synthetic construct
MYDWWPELCALAC

SEQ ID NO: 373 - protein, 11 aa, synthetic construct
  SITE 7: note = X is L-penicillamine
  SITE 8: note = X is 1-amino-isobutyric acid
DWWPELXXLIC

SEQ ID NO: 374 - protein, 11 aa, synthetic construct
  SITE 8: note = X is 1-amino-isobutyric acid
  SITE 11: note = X is L-penicillamine
DWWPELCXLIX

SEQ ID NO: 375 - protein, 11 aa, synthetic construct
  SITE 3: note = X is 3-(1-naphthyl)-L-alanine
  SITE 8: note = X is 1-amino-isobutyric acid
DWXPENCXLIC

SEQ ID NO: 376 - protein, 11 aa, synthetic construct
  SITE 3: note = X is 3-(1-naphthyl)-L-alanine
  SITE 4: note = X is 4,4-difluoro-L-proline
  SITE 8: note = X is 1-amino-isobutyric acid
DWXXELCXLIC

SEQ ID NO: 377 - protein, 11 aa, synthetic construct
  SITE 3: note = X is 3-(1-naphthyl)-L-alanine
  SITE 4: note = X is (R)-nipecotic acid
  SITE 8: note = X is 1-amino-isobutyric acid
DWXXELCXLIC

SEQ ID NO: 378 - protein, 11 aa, synthetic construct
  SITE 3: note = X is 3-(1-naphthyl)-L-alanine
  SITE 4: note = X is (R)-nipecotic acid
  SITE 8: note = X is 1-amino-isobutyric acid
DWXPEXCYLIC

SEQ ID NO: 379 - protein, 11 aa, synthetic construct
  SITE 3: note = X is 3-(1-naphthyl)-L-alanine
  SITE 8: note = X is 1-amino-isobutyric acid
DWXPEFCXLIC

SEQ ID NO: 380 - protein, 11 aa, synthetic construct
  SITE 3: note = X is 3-(1-naphthyl)-L-alanine
  SITE 8: note = X is 1-amino-isobutyric acid
DWXPEKCXLIC

SEQ ID NO: 381 - protein, 11 aa, synthetic construct
  SITE 3: note = X is 3-(1-naphthyl)-L-alanine
  SITE 6: note = X is N-leucine
  SITE 8: note = X is 1-amino-isobutyric acid
DWXPEXCXLIC

SEQ ID NO: 382 - protein, 11 aa, synthetic construct
  SITE 3: note = X is 3-(1-naphthyl)-L-alanine
  SITE 6: note = X is L-citrulline
  SITE 8: note = X is 1-amino-isobutyric acid
DWXPEXCXLIC

SEQ ID NO: 383 - protein, 11 aa, synthetic construct
  SITE 3: note = X is 3-(1-naphthyl)-L-alanine
  SITE 6: note = X is (S)-2,3-Diaminopropionic acid
  SITE 8: note = X is 1-amino-isobutyric acid
DWXPEXCXLIC

SEQ ID NO: 384 - protein, 11 aa, synthetic construct
  SITE 3: note = X is ortho-chloro-L-phenylalanine
  SITE 4: note = 4-(R)-hydroxy-L-proline
  SITE 6: note = X is D-leucine
  SITE 8: note = X is 4-amino-tetrahydropyran-4-carboxylic acid
DWXXEXCXLIC

SEQ ID NO: 385 - protein, 11 aa, synthetic construct
  SITE 3: note = X is para-chloro-L-phenylalanine
  SITE 4: note = X is 4-(R)-hydroxy-L-proline
  SITE 6: note = X is D-leucine
  SITE 8: note = X is 4-amino-tetrahydropyran-4-carboxylic acid
DWXXEXCXLIC

SEQ ID NO: 386 - protein, 11 aa, synthetic construct
  SITE 3: note = X is para-chloro-L-phenylalanine
  SITE 4: note = X is 4-(R)-hydroxy-L-proline
  SITE 6: note = X is D-leucine
  SITE 8: note = X is 4-amino-tetrahydropyran-4-carboxylic acid
DWXXEXCXLIC

SEQ ID NO: 387 - protein, 11 aa, synthetic construct
  SITE 4: note = X is 4-(R)-hydroxy-L-proline
  SITE 6: note = X is D-leucine
  SITE 8: note = X is 4-amino-tetrahydropyran-4-carboxylic acid
DWFXEXCXLIC

SEQ ID NO: 388 - protein, 11 aa, synthetic construct
  SITE 3: note = X is 5-chloro-L-tryptophan
  SITE 4: note = X is4-(R)-hydroxy-L-proline
  SITE 6: note = X is D-leucine
  SITE 8: note = X is 4-amino-tetrahydropyran-4-carboxylic acid
DWXXEXCXLRC

SEQ ID NO: 389 - protein, 11 aa, synthetic construct
  SITE 3: note = X is 5-chloro-L-tryptophan
  SITE 4: note = X is 4-(R)-hydroxy-L-proline
  SITE 6: note = X is D-leucine
  SITE 8: note = X is 4-amino-tetrahydropyran-4-carboxylic acid
DWXXEXCXLEC

SEQ ID NO: 390 - protein, 11 aa, synthetic construct
  SITE 3: note = X is 5-chloro-L-tryptophan
  SITE 4: note = X is 4-(R)-hydroxy-L-proline
  SITE 6: note = X is D-leucine
  SITE 8: note = X is 4-amino-tetrahydropyran-4-carboxylic acid
DWXXEXCXLWC

SEQ ID NO: 391 - protein, 11 aa, synthetic construct
  SITE 2: note = X is 3,4-dichlor-L-phenylalanine
  SITE 3: note = X is 3,4-dichlor-L-phenylalanine
  SITE 4: note = X is (2S,3aS,7aS)- octahydroindolecarboxylic acid
  SITE 6: note = X is D-leucine
  SITE 8: note = X is 4-amino-tetrahydropyran-4-carboxylic acid
DXXXEXCXLIC

SEQ ID NO: 392 - protein, 11 aa, synthetic construct
  SITE 2: note = X is 3,4-dichlor-L-phenylalanine
  SITE 3: note = X is 3,4-dichlor-L-phenylalanine
  SITE 4: note = X is N-methyl-glycine
  SITE 6: note = X is D-leucine
  SITE 8: note = X is 4-amino-tetrahydropyran-4-carboxylic acid
DXXXEXCXLIC

SEQ ID NO: 393 - protein, 11 aa, synthetic construct
  SITE 3: note = X is 5-chloro-L-tryptophan
  SITE 4: note = X is 4-(R)-hydroxy-L-proline
  SITE 6: note = X is D-leucine
  SITE 8: note = X is 4-amino-tetrahydropyran-4-carboxylic acid
DWXXEXCXLIC

SEQ ID NO: 394 - protein, 13 aa, synthetic construct
  SITE 11: note = X is S-methyl-L-cysteine
MYDWWPELCALIX

SEQ ID NO: 395 - protein, 13 aa, synthetic construct
  SITE 9: note = X is S-methyl-L-cysteine
MYDWWPELXALIC